(19) United States
(12) Patent Application Publication
(10) Pub. No.: US 2026/0207797 A1
(43) Pub. Date: Jul. 23, 2026

(54) NEUROTENSIN RECEPTOR 1 (NTSR1) TARGETED THERAPEUTICS AND USES THEREOF

(71) Applicant: Radionetics Oncology, Inc., San Diego, CA (US)

(72) Inventors: Yifeng XIONG, San Diego, CA (US); Yunfei ZHU, San Diego, CA (US)

(21) Appl. No.: 19/394,360

(22) Filed: Nov. 19, 2025

Related U.S. Application Data
(60) Provisional application No. 63/723,256, filed on Nov. 21, 2024.
(60) Provisional application No. 63/791,994, filed on Apr. 21, 2025.
(60) Provisional application No. 63/908,948, filed on Oct. 31, 2025.

(51) Int. Cl. A61K 51/04

(52) U.S. Cl. CPC A61K 51/0497; A61K 51/0482

(57) ABSTRACT

Described herein are radiotherapeutics that target tumor cells expressing the Neurotensin Receptor 1 (NTSR1) and their use in the treatment and/or diagnosis of cancer.

### CROSS-REFERENCE TO RELATED APPLICATIONS

This application claims the benefit of U.S. Provisional Patent Application No. 63/723,256, filed Nov. 21, 2024, and U.S. Provisional Patent Application No. 63/791,994, filed Apr. 21, 2025, and U.S. Provisional Patent Application No. 63/908,948, filed Oct. 31, 2025, which are incorporated herein by reference in their entireties.

### FIELD OF THE INVENTION

Described herein are radiotherapeutics that target tumor cells expressing Neurotensin Receptor 1 (NTSR1) and methods of using such radiotherapeutics as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g., adenomas). Malignant neoplasms display features such as genetic mutations, loss of normal function, rapid division, and ability metastasize (invade) to other tissues; and neoplasms of uncertain or unknown behavior. Malignant neoplasms (i.e., cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy.

G protein-coupled receptors (GPCRs) are an important class of cell surface receptors that are frequently overexpressed in tumor cells and considered promising targets for selective tumor therapy. Neurotensin Receptor 1 (NTSR1) is a GPCR overexpressed in several cancers. NTSR1 is upregulated in tumor tissues and promotes cancer progression in many types of cancers including digestive, lung, pancreatic, colorectal, head and neck and breast cancers, lung, pancreatic, colorectal, head and neck and breast cancers. As such, targeted delivery of radionuclides to tumors with NTSR1-targeting conjugates offers a novel approach to treat and diagnose various cancers.

### SUMMARY OF THE INVENTION

Described herein are radiopharmaceuticals for use in the diagnosis and/or treatment of tumors. The present disclosure provides an alternative and improved method for the treatment of tumors by targeting tumors that overexpress the Neurotensin Receptor 1 (NTSR1). In some embodiments, the radiopharmaceuticals disclosed herein are useful in the treatment of tumors that overexpress NTSR1. In some other embodiments, the radiopharmaceuticals disclosed herein are useful in the identification of tissues or organs in a subject comprising tumors overexpressing NTSR1. The radiopharmaceuticals disclosed herein are also useful for the in vivo imaging of a subject for the presence of and distribution of tumors that overexpress NTSR1 in the subject.

In one aspect, described herein is a compound of Formula (A), or a pharmaceutically acceptable salt thereof:

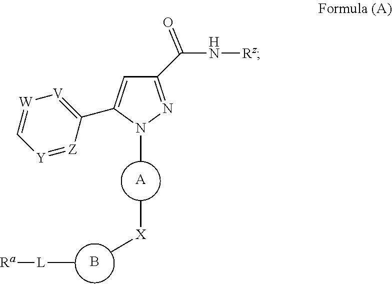

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R² substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R² substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH((═N)OH, —NR⁵R⁶, —NO₂, —NR⁷C(═O)R⁸, —NR⁹C(═O)OR¹⁰, and —OC(═O)NR¹¹R¹²; R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)—, ═S(═NH)(═NH), —CH═CH—C(═O)—, or —CH₂CH₂—C(═O)—;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
W is —C(R^{r})— or —N— and Y is —C(R^{s})— or —N—; wherein R^{r} and R^{s} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—, wherein R^{x} and R^{y} are each independently selected from the group consisting of: —F, —Cl, —Br, —CF₃, —C₁-C₆ alkyl, —O—C₁-C₆ alkyl, —C₃-C₆ cycloalkyl, aryl and heteroaryl.
R^{z} is

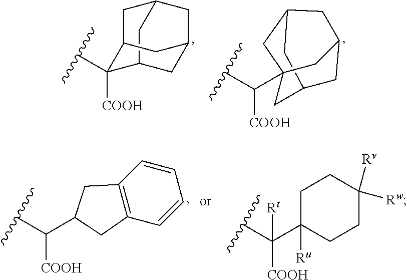

R^{v} and R^{w} are each independently —F, —Cl, or —CH₃ and R^{t} and R^{u} are each independently —C₁-C₄ alkyl; or
R^{v} is —F, —Cl, or —CH₃; R^{t} is —C₁-C₄ alkyl; and R^{u} and R^{w} come together with the carbon atoms to which they are attached to form a cyclopentyl or cyclohexyl ring;
L is a linker; and
R^{a} is a chelating moiety or a radionuclide complex thereof.

In another aspect, described herein is a compound of Formula (I), or a pharmaceutically acceptable salt thereof:

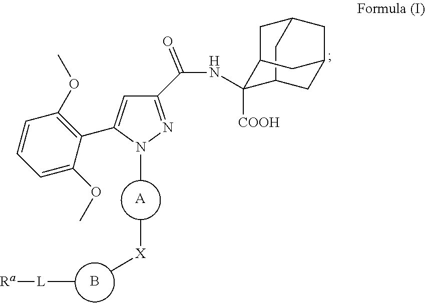

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
X is absent, —O—, —NH—, —S—, —S(═O)(═NH)—, ═S(═NH)(═NH)—, —CH═CH—C(═O)—, or —CH₂CH₂-C(═O)—;
Ring B is absent, piperidinyl, pyrrolidinyl, azetidinyl, piperazinyl, 1,2,3,6-tetrahydropyridinyl, 3,6-diazabicyclo[3.1.1]heptanyl, wherein Ring B is optionally substituted with one or more R² substituents;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
R³, R⁴, R⁵, R⁶, R⁷, R⁸, R⁹, R¹⁰, R¹¹ and R¹² are each independently —H or —CH₃;
L is a linker; and
R^{a} is a chelating moiety or a radionuclide complex thereof.

In some embodiments, Ring A is phenyl, wherein Ring A is optionally substituted with 1-6 R¹ substituents.

In some embodiments, Ring A is tetrahydroisoquinolinyl, wherein Ring A is optionally substituted with 1-6 R¹ substituents.

In some embodiments, R^{a} is a chelating moiety independently selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))-dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, R^{a} is a chelating moiety independently selected from the group consisting of:

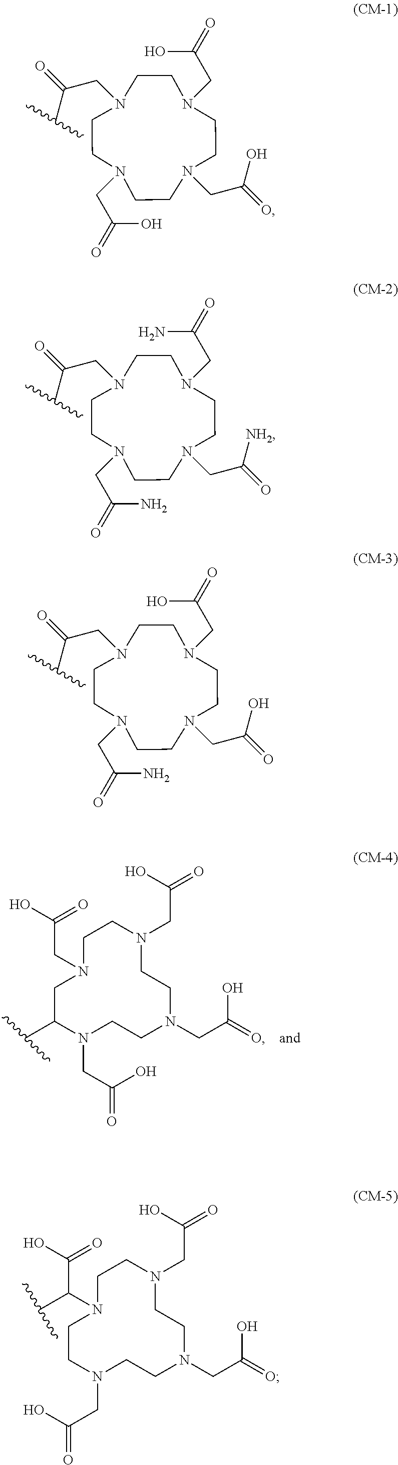 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]

or a radionuclide complex thereof.

In some embodiments, L is: -L², -L⁴-, -L²-L⁴-, -L²-L⁶-, -L²-L³-L⁴-, -L²-L³-L⁶-, -L²-L⁵-L⁶-, -L²-L³-L⁵-L⁶- or -L²-L³-L⁴-L⁵-L⁶-;
L² is absent, —C₁-C₂₀alkylene-, —C₁-C₂₀ alkylene-NR¹³—, —C₁-C₂₀ alkylene-NR¹³—C(═O)—, —NR¹³—C₁-C₂₀ alkylene-, —NR¹³—C₁-C₂₀ alkylene-NR¹⁶—, —C₁-C₂₀ alkylene-O—, —O—C₁-C₂₀ alkylene-, —O—C₁-C₂₀ alkylene-NR¹⁶—, —NR¹³—C(═O)—C₁-C₂₀ alkylene-NR¹⁶—, —C₁-C₂₀ alkylene-C(═O)—, —C(═O)—C₁-C₂₀ alkylene-, —C(═O)—C₁-C₂₀ alkylene-NR¹³—, —C(═O)—NR¹³—C₁-C₂₀ alkylene-NR¹⁶—, —C(═NH)NH—C₁-C₂₀ alkylene-NR¹³—, —C(═NH)NH—C₁-C₂₀ alkylene-, or —C(═O)—NH—O—(CH₂)v-; wherein each C₁-C₂₀ alkylene is optionally substituted with 1-3 substituents selected from the group consisting of —OH, —NH₂, and —COOH;
each R¹³ is independently selected from H or —C₁-C₄ alkyl;
each v is independently 1, 2, 3, 4, 5 or 6;
L³ is absent, —C₁-C₂₀ alkylene-, —C₁-C₂₀ alkylene-NR¹⁴—, —C(═O)—C₁-C₂₀ alkylene-, —(CH₂CH₂O)_{w}, —CH₂CH₂—, —C(═O)—(CH₂CH₂O)_{w}—CH₂CH₂—, —(CH₂CH₂NR¹⁴)_{w}—CH₂CH₂—, —C(═O)NR¹⁴—(CH₂CH₂NR¹⁴)_{w}—CH₂CH₂—, —C(═O)—NH—O—(CH_{2Z})—, or —C(═O)—(CH₂)_{w}—O—NH—(CH₂)—C(═O)—; wherein each C₁-C₂₀ alkylene is optionally substituted with 1-3 substituents selected from the group consisting of —OH and —NH₂;
   each R⁴ is independently H or —C₁-C₆ alkyl;
   each w is independently 1, 2, 3, 4, 5 or 6;
L⁴ is absent, a natural or unnatural amino acid, or a peptide that is formed from two or more independently selected natural and unnatural amino acids, wherein when two or more amino acids are present then the N atom of the amide linking the amino acids is optionally substituted with —C₁-C₆ alkyl;
L⁵ is absent or -L⁷-L⁸-L⁹-;
   L⁷ is absent, —O—, —S—, —NH— or —NCH₃—;
   L⁸ is substituted or unsubstituted cycloalkylene, substituted or unsubstituted cycloalkenylene, or substituted or unsubstituted heterocycloalkylene;
   L⁹ is absent, —(CH₂)ₓ—, —C(═O)(CH₂)x-, or —NR¹⁵(CH₂)—;
      x is 1, 2, 3, 4, 5 or 6;
   each R¹⁵ is independently selected from H or —C₁-C₆ alkyl; and
L⁶ is absent, —NH—, or —N(CH₃)—;
wherein if L⁶ is absent, L⁴ cannot be absent. In some embodiments, L is: -L², -L-, -L²-L-, -L²-L⁶-, -L³-L⁶-, -L²-L³-L⁴-, -L²-L³-L⁶-, -L²-L⁵-L⁶-, -L²-L³-L⁵-L⁶- or -L²-L³-L⁴-L⁵-L⁶-;
   L² is absent, —C₁-C₂₀ alkylene, —C₁-C₂₀ alkylene-NR¹³—, —C₁-C₂₀ alkylene-NR¹³—C(═O)—, —NR¹³—C₁-C₂₀ alkylene-, —NR¹³—C₁-C₂₀ alkylene-NR¹⁶—, —C₁-C₂₀ alkylene-O—, —O—C₁-C₂₀ alkylene-, —O—C₁-C₂₀ alkylene-NR¹⁶—, —NR¹³—C(═O)—C₁-C₂₀ alkylene-NR¹⁶—, —C₁-C₂₀ alkylene-C(═O)—, —C(═O)—C₁-C₂₀ alkylene-, —C(═O)—C₁-C₂₀ alkylene-NR¹³—, —C(═O)—NR¹³—C₁-C_{2M} alkylene-NR¹⁶—, —C(═NH)NH—C₁-C₂₀ alkylene-NR¹³—, —C(═NH)NH—C₁-C₂₀ alkylene-, or —C(═O)—NH—O—(CH₂)ᵥ—; wherein each C₁-C₂₀ alkylene is optionally substituted with 1-3 substituents selected from the group consisting of —OH, —NH₂, and —COOH;
      each R¹³ and R¹⁶ are independently selected from H or —C₁-C₄ alkyl;
      each v is independently 1, 2, 3, 4, 5 or 6;
   L³ is absent, —C₁-C₂₀ alkylene-, —C₁-C₂₀ alkylene-NR¹⁴—, —C(═O)—C₁-C₂₀ alkylene-, —(CH₂CH₂O)_{w}—CH₂CH₂—, —C(═O)—(CH₂CH₂O)_{w}—CH₂CH₂—, —(CH₂CH₂NR¹⁴)_{w}—CH₂CH₂—, —C(═O)NR¹⁴—(CH₂CH₂NR¹⁴)_{w}—CH₂CH₂—, —C(═O)—NH—O—(CH₂)_{w}—, or —C(═O)—(CH₂)_{w}—O—NH—(CH₂)_{w}—C(═O)—; wherein each C₁-C₂₀ alkylene is optionally substituted with 1-3 substituents selected from the group consisting of —OH and —NH₂;
      each R⁴ is independently H or —C₁-C₆ alkyl;
      each w is independently 1, 2, 3, 4, 5 or 6;
   L⁴ is absent, a natural or unnatural amino acid, or a peptide that is formed from two or more independently selected natural and unnatural amino acids, wherein when two or more amino acids are present then the N atom of the amide linking the amino acids is optionally substituted with —C₁-C₆ alkyl;
   L⁵ is absent or -L⁷-L⁸-L⁹-;
      L⁷ is absent, —O—, —S—, —NH— or —NCH₃—;
      L⁸ is substituted or unsubstituted cycloalkylene, substituted or unsubstituted cycloalkenylene, or substituted or unsubstituted heterocycloalkylene;
      L⁹ is absent, —(CH₂)ₓ—, —C(═O)(CH₂)ₓ—, or —NR¹⁵(CH₂)x-;
         x is 1, 2, 3, 4, 5 or 6;
      each R¹⁵ is independently selected from H or —C₁-C₆ alkyl; and
   L, is absent, —NH—, or —N(CH₃)—;
   wherein if L⁶ is absent, L⁴ cannot be absent.

In some embodiments, the radionuclide of the radionuclide complex is a lanthanide or an actinide. In some embodiments, the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium. In some embodiments, the radionuclide of the radionuclide complex is a diagnostic or therapeutic radionuclide. In some embodiments, the radionuclide of the radionuclide complex is: an Auger electron-emitting radionuclide; or an α-emitting radionuclide; or a β-emitting radionuclide; or a γ-emitting radionuclide. In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²¹⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

Also described herein is a pharmaceutical composition comprising a compound described herein (e.g., a compound of Formula (A) or Formula (I)), or a pharmaceutically acceptable salt thereof, and at least one pharmaceutically acceptable excipient. In some embodiments, the pharmaceutical composition is formulated for administration to a mammal by intravenous administration or subcutaneous administration. In some embodiments, the pharmaceutical composition is formulated for administration to a mammal by intravenous administration.

In another aspect, described herein is a method for the treatment of cancer comprising administering to a mammal with cancer an effective amount of a compound described herein (e.g., a compound of Formula (A) or Formula (I)), or a pharmaceutically acceptable salt thereof, or an effective amount of pharmaceutical composition comprising a compound described herein (e.g., a compound of Formula (A) or Formula (I)), or a pharmaceutically acceptable salt thereof. In some embodiments, the cancer comprises tumors and the tumors overexpress the Neurotensin Receptor 1 (NTSR1). In some embodiments, the cancer is lung cancer, pancreatic cancer, colorectal cancer, head and neck cancers, breast cancer, prostate cancer, gastrointestinal cancer, or bone cancer. In some embodiments, the cancer is lung cancer, pancreatic cancer, colorectal cancer, head and neck cancers, breast cancer or prostate cancer. In some embodiments, the cancer is lung cancer. In some embodiments, the cancer is pancreatic cancer. In some embodiments, the cancer is pancreatic ductal adenocarcinoma (PDAC). In some embodiments, the cancer is colorectal cancer. In some embodiments, the cancer is colorectal adenocarcinoma (CRC). In some embodiments, the cancer is head and neck cancer. In some embodiments, the cancer is squamous-cell carcinoma of head and neck (SCCHN). In some embodiments, the cancer is breast cancer. In some embodiments, the cancer is prostate cancer. In some embodiments, the lung cancer is non-small cell lung cancer. In some embodiments, the cancer is gastrointestinal cancer. In some embodiments, the cancer is gastrointestinal stromal tumors (GIST). In some embodiments, the cancer is stomach cancer. In some embodiments, the cancer is gastric adenocarcinoma. In some embodiments, the cancer is bone cancer. In some embodiments, the cancer is Ewing Sarcoma (ES).

In another aspect, described herein is a method for treating tumors in a mammal with a radionuclide comprising administering to the mammal a compound described herein (e.g., a compound of Formula (A) or Formula (I)), or a pharmaceutically acceptable salt thereof, or a pharmaceutical composition comprising a compound described herein (e.g., a compound of Formula (A) or Formula (I)), or a pharmaceutically acceptable salt thereof. In some embodiments, the mammal has been diagnosed with lung cancer. In some embodiments, the mammal has been diagnosed with colorectal cancer. In some embodiments, the mammal has been diagnosed with pancreatic cancer. In some embodiments, the mammal has been diagnosed with head and neck cancers. In some embodiments, the mammal has been diagnosed with breast cancer. In some embodiments, the mammal has been diagnosed with prostate cancer.

In another aspect, described herein is a method of targeting delivery of a radionuclide to tumors in a mammal comprising administering to a mammal with tumors a compound described herein (e.g., a compound of Formula (A) or Formula (I)), or a pharmaceutically acceptable salt thereof, or a pharmaceutical composition comprising a compound described herein (e.g., a compound of Formula (A) or Formula (I)), or a pharmaceutically acceptable salt thereof; wherein the tumors overexpress the Neurotensin Receptor 1 (NTSR1).

In another aspect, described herein is a method for identifying tissues or organs in a mammal with tumors expressing the Neurotensin Receptor 1 (NTSR1) comprising administering to the mammal a compound described herein (e.g., a compound of Formula (A) or Formula (I)), or a pharmaceutically acceptable salt thereof, or a pharmaceutical composition comprising a compound described herein (e.g., a compound of Formula (A) or Formula (I)), or a pharmaceutically acceptable salt thereof; and performing positron emission tomography (PET) analysis, single-photon emission computerized tomography (SPECT), or magnetic resonance imaging (MRI); wherein R^{a} is a chelating moiety-diagnostic radionuclide complex.

In yet another aspect, described herein is a method for the in vivo imaging of tissues or organs in a mammal with tumors expressing the Neurotensin Receptor 1 (NTSR1) comprising administering to the mammal a compound described herein (e.g., a compound of Formula (A) or Formula (I)), or a pharmaceutically acceptable salt thereof, or a pharmaceutical composition comprising a compound described herein (e.g., a compound of Formula (A) or Formula (I)), or a pharmaceutically acceptable salt thereof; and performing positron emission tomography (PET) analysis, single-photon emission computerized tomography (SPECT), or magnetic resonance imaging (MRI); wherein R^{a} is a chelating moiety-diagnostic radionuclide complex.

In one aspect, described herein is a compound of Formula (D), or a pharmaceutically acceptable salt thereof:

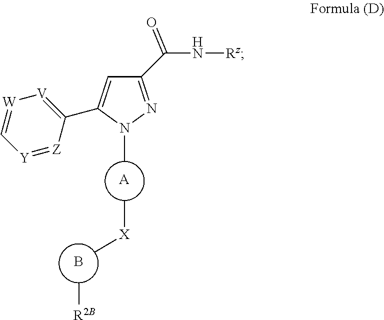

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O);
wherein any saturated carbon of Ring A is optionally substituted with oxo (═O); wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)—, ═S(═NH)(═NH), —CH═CH—C(═O)—, or —CH₂CH₂—C(═O)—;
each R^{2A} is independently selected from the group consisting of hydrogen, —F, —Cl, —Br, —I, —OH, —O—C₁-C₆ alkyl, substituted or unsubstituted C₁-C₆ alkyl, C₁-C₆ fluoroalkyl, substituted or unsubstituted heteroalkyl, —CN, —CO₂H, —C(═O)NR^{3A}R^{4A}, —CH(═N)OH, —NR^{5A}R^{6A}, —NO₂, —NR^{7A}C(═O)R^{8A}, —NR^{9A}C(═O)OR^{10A}, and —OC(═O)NR^{1A}R^{12A}, or two R^{2A} variables on the same or adjacent carbon atoms come together to form an unsubstituted C₃-C₆ cycloalkyl ring;
R^{3A}, R^{4A}, R^{5A}, R^{6A}, R^{7A}, R^{5A}, R^{9A}, R^{10A}, R^{11A} and R^{12A} are each independently —H or —CH₃;
R^{2B} is hydrogen, —R^{b}, —U—R^{c}, or —NH—R^{d},
   R^{b} is hydrogen, C₁-C₆ alkyl or 6-membered heteroalkyl;
   U is —O— or —NH—;
   R^{c} is hydrogen or C₁-C₆ alkyl;
   R^{d} is C₁-C₆ alkyl;
W is —C(R^{r})— or —N— and Y is —C(R^{t})— or —N—; wherein R^{r} and R^{t} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—; wherein R^{x} and R^{y} are each independently selected from the group consisting of: —F, —Cl, —Br, —CF₃, —C₁-C₆ alkyl, —O—C₁-C₆ alkyl, —C₃-C₆ cycloalkyl, aryl and heteroaryl;
R^{z} is:

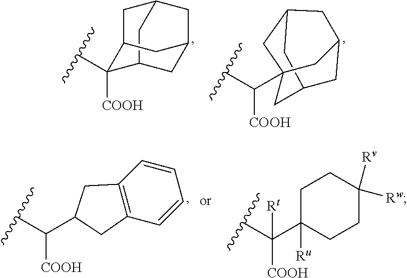

R^{v} and R^{w} are each independently —F, —Cl, or —CH₃ and R^{t} and R^{u} are each independently —C₁-C₄ alkyl; or
R^{v} is —F, —Cl, or —CH₃; R^{t} is —C₁-C₄ alkyl; and R^{u} and R^{w} come together with the carbon atoms to which they are attached to form a cyclopentyl or cyclohexyl ring.

Also described herein is a pharmaceutical composition comprising a compound described herein (e.g., a compound of Formula (D) or Formula (E)), or a pharmaceutically acceptable salt thereof, and at least one pharmaceutically acceptable excipient. In some embodiments, the pharmaceutical composition is formulated for administration to a mammal by oral administration.

In another aspect, described herein is a method for the treatment of cancer comprising administering to a mammal with a disease an effective amount of a compound described herein (e.g., a compound of Formula (D) or Formula (E)), or a pharmaceutically acceptable salt thereof, or an effective amount of pharmaceutical composition comprising a compound described herein (e.g., a compound of Formula (D) or Formula (E)), or a pharmaceutically acceptable salt thereof. In some embodiments, the disease is a psychological disorder, a movement disorder, a gastrointestinal disorder, a cardiovascular disorder, a metabolic disorder, or pain management.

In some embodiments, the psychological disorder is psychosis or schizophrenia.

In some embodiments, the movement disorder is Parkinson's disease.

In some embodiments, the gastrointestinal disorder is irritable bowel syndrome (IBS), diarrhea, colitis, ulcers, tumors of the GI tract, dyspepsia, pancreatitis, or esophagitis.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 depicts the efficacy of ¹⁷⁷Lu[Lu] complexes of Compound I-7, Compound 15, Compound 116, and Compound 142 in female Swiss nude mice. Animals were dosed with vehicle, 90, 45, or 15 MBq of ¹⁷⁷Lu-radiolabeled complexes (at 0.4 total nmol of each compound). Tumors were measured twice weekly for a total of 60 days post tumor cell inoculation.

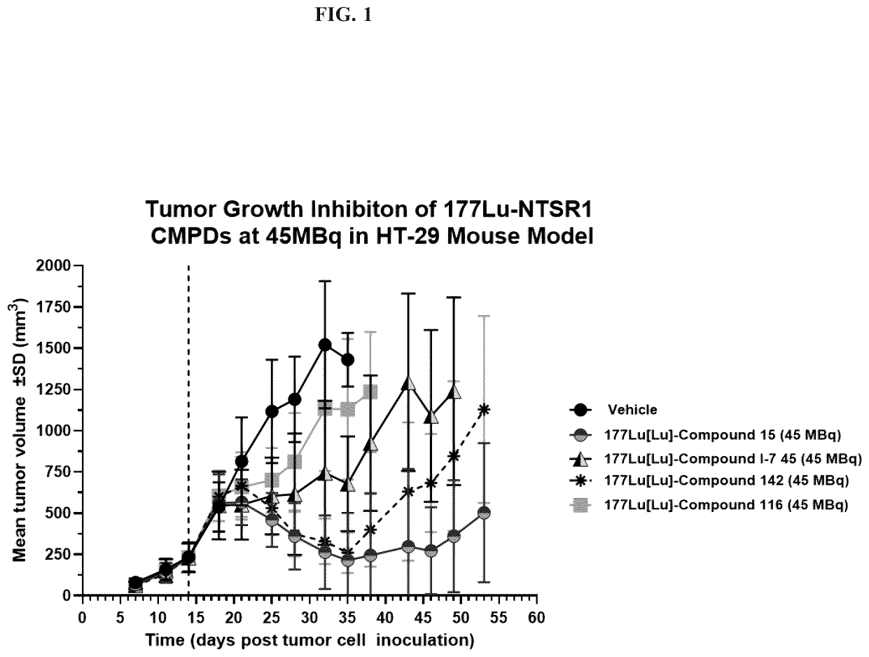

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e. do not display malignant features and are generally unlikely to become dangerous such as adenomas), malignant (i.e. display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often require multiple interventions and are often done in combination with radiation and chemotherapy. Chemotherapy proves to be a potent weapon in the fight against cancer in many cases. Chemotherapy is typically performed by systemic administration of potent cytotoxic drugs, but these compounds often lack tumor selectivity and therefore also kill healthy cells in the body. The resulting non-specific toxicity is the cause of severe side effects of chemotherapy which occur because chemotherapy does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Like chemotherapy, many radiation therapy options also lack tumor cell identification properties needed to achieve the ultimate goal of targeted tumor therapy with drug molecules or radionuclides.

Described herein are radiopharmaceuticals that selectively deliver radionuclides to malignant cells that overexpress NTSR1 for use in cancer detection, image guided cancer surgery, and selective tumor killing.

### The Neurotensin Receptor 1 (NTSR1)

Neurotensin (NT) is an endogenous 13-amino acids peptide distributed in the central nervous system (CNS) and in peripheral tissues. NT shows a wide range of biological activities: in the brain, it is involved in the dopaminergic system modulation, hypothermia and analgesia, while in periphery it modulates digestive and cardiovascular functions and proinflammatory responses. NT effects occur via the activation of the neurotensin receptors NTSR1 and NTSR2, belonging to the GPCR family, and NTSR3, a receptor with single transmembrane domain. The natural ligand NT has high affinity and specificity to these receptors and it has been demonstrated that the shortest active sequence is the fragment NT[8-13], which is the active portion of the neurotensin peptide, consisting of only the 8th to 13th amino acids in its sequence.

Neurotensin receptor 1 (NTSR1) belongs to the family of neurotensin receptors (NTSRs), which modulate the effects of the neuropeptide hormone neurotensin in the gastrointestinal system. NTSR1 is the primary mediator of neurotensin signaling due to its sub-nanomolar affinity to its natural ligand. Overexpression of NTSR1 has been associated with disease progression of multiple types of cancers, including lung cancer (e.g. non-small cell lung cancer), pancreatic cancer (e.g. pancreatic adenocarcinoma), colorectal cancer, head and neck cancers, breast cancer (e.g. invasive ductal breast carcinoma), and prostate cancer, making it a promising target for diagnostic imaging and radioligand therapy.

NT represents a peptide of choice to develop tumor targeting vectors for the elaboration of diagnostic and therapeutic agents. However, the high sensitivity of NT to peptidases, that should be countered while preserving the fast pharmacokinetics and high tumor uptake necessary for imaging procedures, make the design of peptide analogues challenging. Non-peptide compounds, that are intrinsically resistant to proteases, are an attractive alternative to peptide analogs.

Radiopharmaceuticals targeting NTSR1 are important for the development of new cancer therapies.

Solid Tumors: Benign and/or Malignant Neoplasms (Cancer)

In one aspect, the NTSR1-targeted radiopharmaceuticals described herein are used to treat benign and/or malignant neoplasms (solid tumors), wherein the neoplasm comprises cells that overexpress NTSR1 on the cell surface.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

In some embodiments, the NTSR1-targeted radiopharmaceuticals described herein are used to treat an adenoma. An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others. Even though benign, they have the potential to cause serious health complications by compressing other structures (mass effect) and by producing large amounts of hormones in an unregulated, non-feedback-dependent manner (causing paraneoplastic syndromes). Overtime adenomas may transform to become malignant, at which point they are called adenocarcinomas.

Adenomas may be found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas; some secrete hormones such as cortisol, causing Cushings syndrome, aldosterone causing Conn's syndrome, or androgens causing hyperandrogenism), thyroid (e.g. thyroid adenoma), pituitary (e.g. pituitary adenomas, such as prolactinoma, Cushing's disease and acromegaly), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis is the spread of malignant cells to new areas of the body, often by way of the lymph system or blood stream. A metastatic tumor is one that has spread from the primary site of origin, or where it started, into different areas of the body. Metastatic tumors comprise malignant cells that may express cell surface NTSR1.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing NTSR1, wherein the tumor is a primary or metastatic tumor. In some embodiments, the tumor to be treated comprises tumor cells expressing NTSR1, wherein the tumor is a primary or metastatic tumor of lung origin. In some embodiments, the tumor to be treated comprises tumor cells expressing NTSR1, wherein the tumor is a primary or metastatic tumor of pancreatic origin. In some embodiments, the tumor to be treated comprises tumor cells expressing NTSR1, wherein the tumor is a primary or metastatic tumor of prostate origin. In some embodiments, the tumor to be treated comprises tumor cells expressing NTSR1, wherein the tumor is a primary or metastatic tumor of breast origin. In some embodiments, the tumor to be treated comprises tumor cells expressing NTSR1, wherein the tumor is a primary or metastatic tumor of colorectal origin. In some embodiments, the tumor to be treated comprises tumor cells expressing NTSR1, wherein the tumor is a primary or metastatic tumor of head or neck origin.

In some embodiments, the NTSR1-targeted radiopharmaceuticals described herein are used to treat a carcinoma. Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc. In some embodiments, the NTSR1-targeted radiopharmaceuticals described herein are used to treat lung carcinoma. In some embodiments, the NTSR1-targeted radiopharmaceuticals described herein are used to treat colorectal carcinoma. In some embodiments, the NTSR1-targeted radiopharmaceuticals described herein are used to treat pancreatic carcinoma. In some embodiments, the NTSR1R-targeted radiopharmaceuticals described herein are used to treat breast carcinoma.

In some embodiments, the NTSR1-targeted radiopharmaceuticals described herein are used to treat a sarcoma. Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Representative Neurotensin Receptor 1 (NTSR1) Targeting Conjugates

In some embodiments, the NTSR1-targeted radiopharmaceuticals described herein have an affinity to NTSR1 that is at least 10-fold, at least 50-fold, at least 100-fold, at least 200-fold, at least 500-fold, or at least 1000-fold greater than the affinity for other non-target receptors.

In some embodiments, the NTSR1-targeted radiopharmaceuticals described herein preferentially accumulate in tumor tissues that express the targeted NTSR1. In some embodiments, the NTSR1-targeted radiopharmaceuticals described herein preferentially accumulate in tissues or organs comprising tumor cells that express NTSR1 as compared to tissues or organ(s) lacking tumor cells that express NTSR1. In some embodiments, the NTSR1-targeted radiopharmaceuticals described herein preferentially accumulate at least 1-fold, at least 2-fold, at least 3-fold, at least 4-fold, at least 5-fold, or greater than 5-fold more in tissues or organ(s) comprising tumor cells that express NTSR1 as compared to tissues or organs lacking tumor cells that express NTSR1. It is understood that the compound may accumulate in certain tissues and organs involved in the metabolism and/or excretion of therapeutics, including but not limited to the kidneys and liver.

In one aspect, described herein is a compound of Formula (A), or a pharmaceutically acceptable salt thereof:

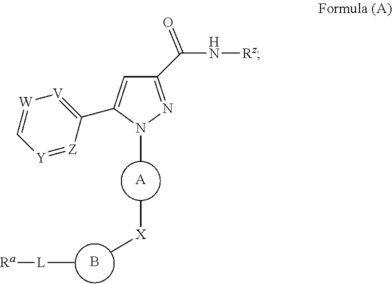

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R² substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R² substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)—, ═S(═NH)(═NH), —CH═CH—C(═O)—, or —C₁H₂CH₂—C(═O)—;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
W is —C(R^{r})— or —N— and Y is —C(R^{s})— or —N—; wherein R^{r} and R^{s} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—, wherein R^{x} and R^{y} are each independently selected from the group consisting of: —F, —Cl, —Br, —CF₃, —C₁-C₆ alkyl, —O—C₁-C₆ alkyl, —C₃-C₆ cycloalkyl, aryl and heteroaryl.
R^{z} is

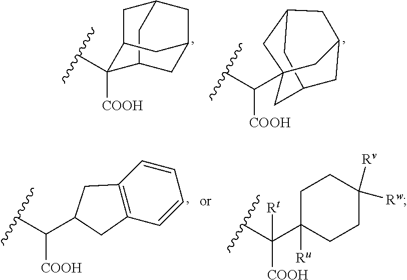

R^{v} and R^{w} are each independently —F, —Cl, or —CH₃ and R^{t} and R^{u} are each independently —C₁-C₄ alkyl; or
R^{v} is —F, —Cl, or —CH₃; R^{t} is —C₁-C₄ alkyl; and R^{u} and R^{w} come together with the carbon atoms to which they are attached to form a cyclopentyl or cyclohexyl ring
L is a linker; and
R^{a} is a chelating moiety or a radionuclide complex thereof.

In some embodiments, described herein is a compound of Formula (A), or a pharmaceutically acceptable salt thereof:

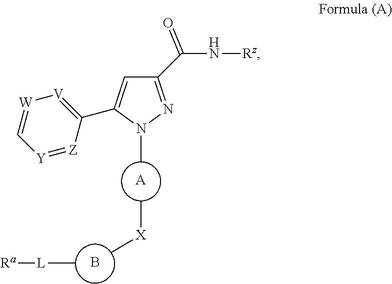

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
X is absent, —O—, —NH—, —S—, —S(═O)(═NH)—, ═S(═NH)(═NH), —CH═CH—C(═O)—, or —CH₂CH₂—C(═O)—;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
R³, R⁴, R⁵, R⁶, R⁷, R⁸, R⁹, R¹⁰, R¹¹ and R¹² are each independently —H or —CH₃;
W and Y are each independently —CH— or —N—;
V is —C(R^{x})— or —N— and Z is —C(R^{y})— or —N—, wherein R^{x} and R^{y} are each independently selected from the group consisting of: —F, —Cl, —Br, —CF₃, —C₁-C₆ alkyl, —O—C₁-C₆ alkyl, —C₃-C₆ cycloalkyl, aryl and heteroaryl.
R^{z} is

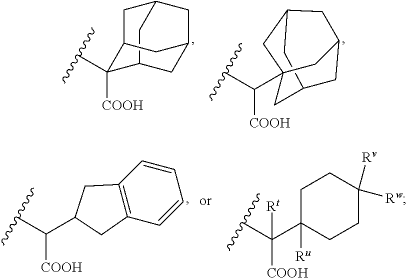

R^{v} and R^{w} are each independently —F, —Cl, or —CH₃;
R^{t} and R^{u} are each independently —C₁-C₄ alkyl;
L is a linker; and
R^{a} is a chelating moiety or a radionuclide complex thereof.

In some embodiments, the compound has the following structure, or a pharmaceutically acceptable salt thereof:

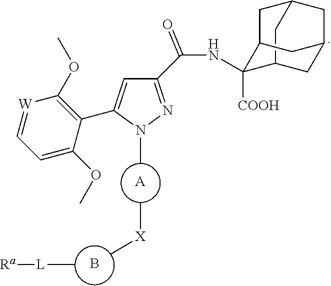

In another aspect, described herein is a compound of Formula (I), or a pharmaceutically acceptable salt thereof:

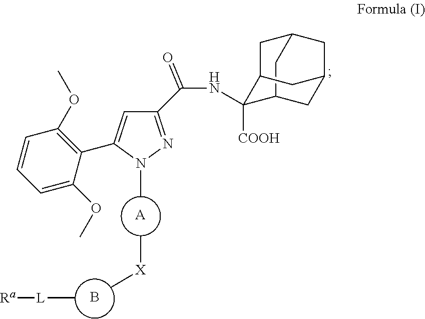

wherein:
   Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
X is absent, —O—, —NH—, —S—, —S(═O)(═NH)—, ═S(═NH)(═NH), —CH═CH—C(═O)—, or —CH₂CH₂—C(═O)—;
Ring B is absent, piperidinyl, pyrrolidinyl, azetidinyl, piperazinyl, 1,2,3,6-tetrahydropyridinyl, 3,6-diazabicyclo[3.1.1]heptanyl, wherein Ring B is optionally substituted with one or more R² substituents;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
R³, R⁴, R⁵, R⁶, R⁷, R⁸, R⁹, R¹⁰, R¹¹ and R¹² are each independently —H or —CH₃;
L is a linker; and
R^{a} is a chelating moiety or a radionuclide complex thereof.

In another aspect, described herein is a compound of Formula (I), or a pharmaceutically acceptable salt thereof:

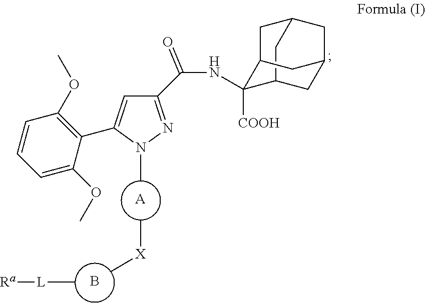

wherein:
   Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents;
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents;
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —C(═O)NR³R⁴, —CH(═N)OH, —NR⁵R⁶, —NO₂, —NR⁷C(═O)R⁸, —NR⁹C(═O)OR¹⁰, and —OC(O)NR¹¹R¹²;
X is absent, —O—, —NH—, —S—, —S(═O)(═NH)—, ═S(═NH═NH), —CH═CH—C(═O)—, or —CH₂CH₂—C(═O)—;
Ring B is absent, piperidinyl, pyrrolidinyl, azetidinyl, piperazinyl, 1,2,3,6-tetrahydropyridinyl, 3,6-diazabicyclo[3.1.1]heptanyl, wherein Ring B is optionally substituted with one or more R² substituents;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
R³, R⁴, R⁵, R⁶, R⁷, R⁸, R⁹, R¹⁰, R¹¹ and R¹² are each independently —H or —CH₃;
L is a linker; and
R^{a} is a chelating moiety or a radionuclide complex thereof.

In some embodiments, W is —N— and Y is —CH—. In some embodiments, W and Y are —CH—.

In some embodiments, V is —C(R^{x})— and Z is —C(R^{y})—.

In some embodiments, R^{x} is —F, —Cl, —CF₃, —CH₃, —CH₂CH₃, —OCH₃, cyclopropyl, or 5 to 6-membered heteroaryl. In some embodiments, R^{x} is pyrimidinyl. In some embodiments, R^{x} is

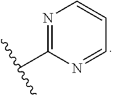 [SMILES: *c1ncccn1]

In some embodiments, R^{y} is —OCH³.

In some embodiments, R^{x} and R^{y} are each independently selected from the group consisting of: —F, —Cl, —Br, —CF₃, —C₁-C₆ alkyl, and —O—C₁-C₆ alkyl. In some embodiments, R^{x} is —F, —Cl, —CF₃, —CH₃, —CH₂CH₃, —OCH₃, cyclopropyl, or pyrimidinyl and R^{y} is —OCH₃. In some embodiments, R^{x} is —F, —Cl, or —OCH₃, and R^{y} is —OCH₃.

In some embodiments, W is —CH— or —N—; Y is —CH—; R^{x} is —OCH₃, and R^{y} is —OCH₃.

In some embodiments, W is —C(H)—, —C(F)—, —C(OMe)-, —C(OEt)-, or —N—.

In some embodiments, Y is —C(H)—, —C(F)—, —C(OMe)-, —C(OEt)-, or —N—.

In some embodiments, V is —C(R^{x})—; Z is —C(R^{y})—; R^{x} is selected from the group consisting of: —F, —Cl, —CF₃, —C₁-C₆ alkyl, —O—C₁-C₆ alkyl, —C₃-C₅ cycloalkyl, and heteroaryl; and R^{y} is —O—C₁-C₆ alkyl. In some embodiments, V is —C(R^{x})—; Z is —C(R^{y})—; and R^{x} and R^{y} are each independently —O—C₁-C₆ alkyl.

In some embodiments, R^{z} is

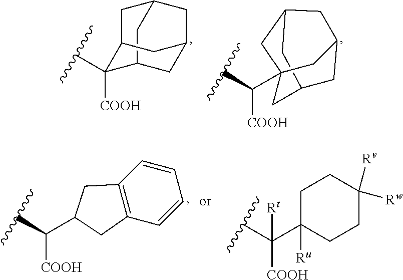

In some embodiments, R^{z} is

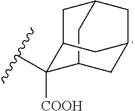 [SMILES: *C1(C(=O)O)C2CC3CC(C2)CC1C3]

In some embodiments, R^{z} is

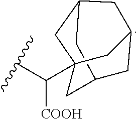 [SMILES: *C(C(=O)O)C12CC3CC(CC(C3)C1)C2]

In some embodiments, R^{z} is

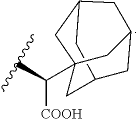 [SMILES: *[C@H](C(=O)O)C12CC3CC(CC(C3)C1)C2]

In some embodiments, R^{z} is

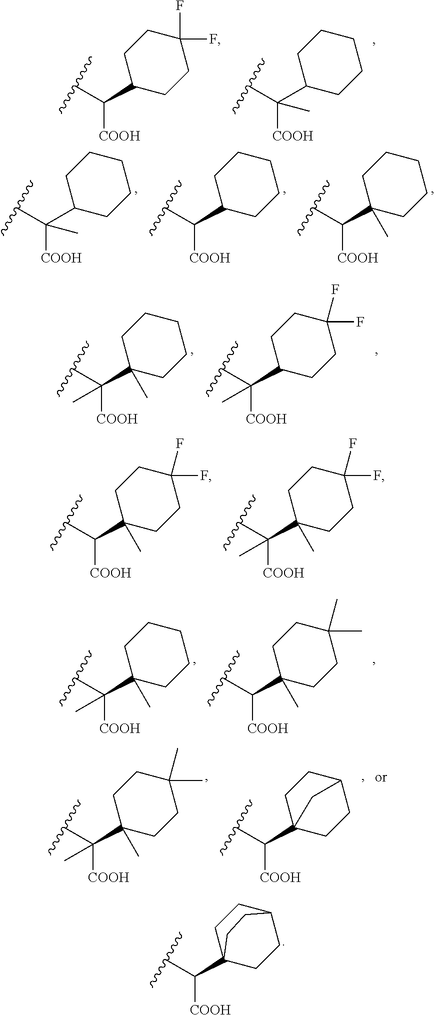

In some embodiments, R^{z} is

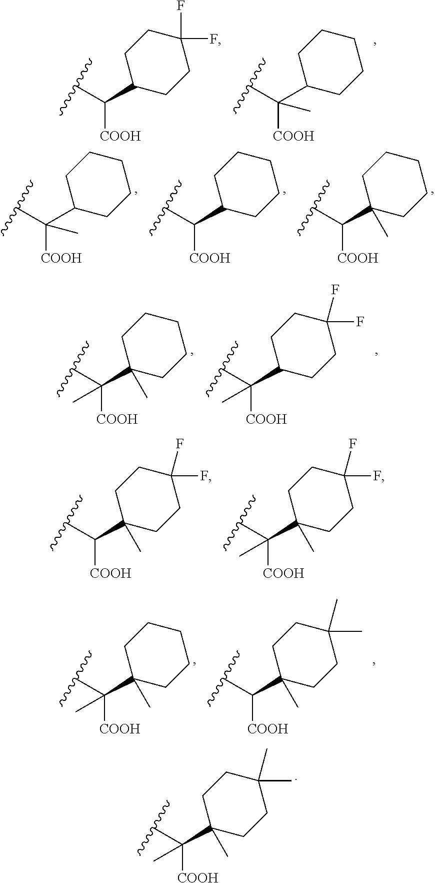

In some embodiments, R^{z} is

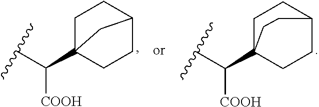 [SMILES: *[C@H](C(=O)O)C12CCC(CC1)C2 ; *[C@H](C(=O)O)C12CCC(CC1)CC2]

In some embodiments, R^{z} is

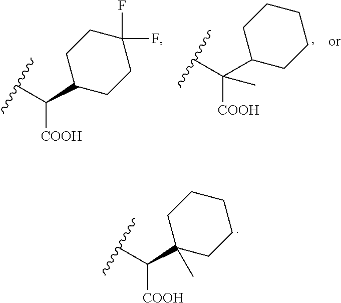

In some embodiments, R^{z} is

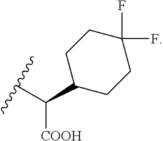 [SMILES: *[C@H](C(=O)O)C1CCC(F)(F)CC1]

In some embodiments, R^{z} is

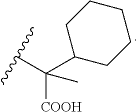 [SMILES: *C(C)(C(=O)O)C1CCCCC1]

In some embodiments, R^{z} is

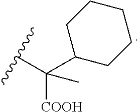 [SMILES: *C(C)(C(=O)O)C1CCCCC1]

In some embodiments, R^{z} is

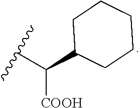 [SMILES: *[C@H](C(=O)O)C1CCCCC1]

In some embodiments, R^{z} is

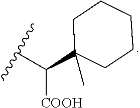 [SMILES: *[C@H](C(=O)O)C1(C)CCCCC1]

In some embodiments, R^{z} is

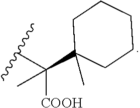 [SMILES: *[C@@](C)(C(=O)O)C1(C)CCCCC1]

In some embodiments, R^{z} is

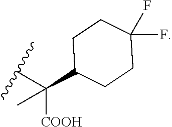 [SMILES: *[C@@](C)(C(=O)O)C1CCC(F)(F)CC1]

In some embodiments, R^{z} is

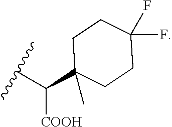 [SMILES: *[C@H](C(=O)O)C1(C)CCC(F)(F)CC1]

In some embodiments, R^{z} is

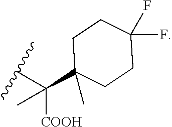 [SMILES: *[C@@](C)(C(=O)O)C1(C)CCC(F)(F)CC1]

In some embodiments, R^{z} is

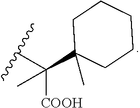 [SMILES: *[C@@](C)(C(=O)O)C1(C)CCCCC1]

In some embodiments, R^{z} is

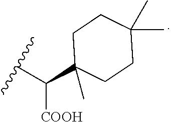 [SMILES: *[C@H](C(=O)O)C1(C)CCC(C)(C)CC1]

In some embodiments, R^{z} is

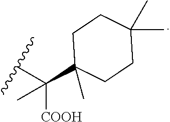 [SMILES: *[C@@](C)(C(=O)O)C1(C)CCC(C)(C)CC1]

In some embodiments, R^{z} is

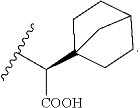 [SMILES: *[C@H](C(=O)O)C12CCC(CC1)C2]

In some embodiments, R^{z} is

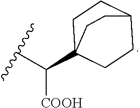 [SMILES: *[C@H](C(=O)O)C12CCC(CC1)CC2]

In another aspect, described herein is a compound of Formula (I), or a pharmaceutically acceptable salt thereof:

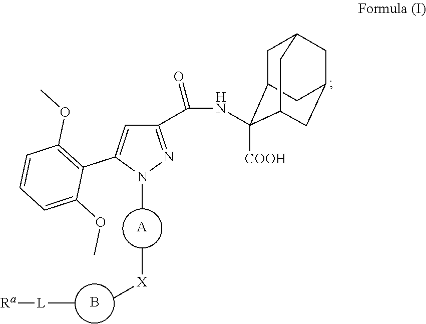

wherein:
Ring A is phenyl, naphthyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, or 1,5-naphthyridinyl;
or Ring A is a partially unsaturated bicyclic heterocyclic ring selected from indolinyl, isoindolinyl, 1,2-dihydroisoquinolinyl, 1,2,3,4-tetrahydroquinolinyl, 1,2,3,4-tetrahydroisoquinolinyl, 1,2,3,4-tetrahydroquinoxalinyl, 1,2,3,4-tetrahydro-1,8-naphthyridine, 5,6,7,8-tetrahydro-1,7-naphthyridinyl, 5,6,7,8-tetrahydro-1,6-naphthyridinyl, 5,6,7,8-tetrahydro-1,5-naphthyridinyl. 1,2,3,4-tetrahydro-1,6-naphthyridinyl, 1,2,3,4-tetrahydro-2,6-naphthyridinyl, 1,2,3,4-tetrahydro-3,6-naphthyridinyl, or 1,2,3,4-tetrahydro-4,6-naphthyridinyl, or 2,3-dihydrobenzo[d]oxazole;
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
X is absent, —O—, —NH—, —S—, —S(═O)(═NH)—, ═S(═NH)(═NH), —CH═CH—C(═O)—, or —CH₂CH₂—C(═O)—;
Ring B is absent, piperidinyl, pyrrolidinyl, azetidinyl, piperazinyl, 1,2,3,6-tetrahydropyridinyl, 3,6-diazabicyclo[3.1.1]heptanyl, wherein Ring B is optionally substituted with one or more R² substituents;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
R³, R⁴, R⁵, R⁶, R⁷, R⁸, R⁹, R¹⁰, R¹¹ and R¹² are each independently —H or —CH₃;
L is a linker; and
R^{a} is a chelating moiety or a radionuclide complex thereof;
wherein if Ring B is absent, Ring A is a partially unsaturated bicyclic heterocyclic ring.

In some embodiments, Ring A is phenyl, naphthyl, or a bicyclic heteroaryl ring; wherein each Ring A is optionally substituted with 1-6 R¹ substituents.

In some embodiments, Ring A is phenyl, naphthyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, or 1,5-naphthyridinyl; wherein Ring A is optionally substituted with 1-6 R¹ substituents.

In some embodiments, Ring A is a partially unsaturated bicyclic heterocyclic ring; wherein any saturated carbon of Ring A is optionally substituted with oxo (═O) and wherein each Ring A is optionally substituted with 1-6 R¹ substituents.

In some embodiments, Ring A is a partially unsaturated bicyclic heterocyclic ring and Ring B is absent; wherein any saturated carbon of Ring A is optionally substituted with oxo (═O) and wherein each Ring A is optionally substituted with 1-6 R¹ substituents.

In some embodiments, Ring A is a partially unsaturated bicyclic heterocyclic ring selected from indolinyl, isoindolinyl, 1,2-dihydroisoquinolinyl, 1,2,3,4-tetrahydroquinolinyl, 1,2,3,4-tetrahydroisoquinolinyl, 1,2,3,4-tetrahydroquinoxalinyl, 1,2,3,4-tetrahydro-1,8-naphthyridine, 5,6,7,8-tetrahydro-1,7-naphthyridinyl, 5,6,7,8-tetrahydro-1,6-naphthyridinyl, 5,6,7,8-tetrahydro-1,5-naphthyridinyl, 1,2,3,4-tetrahydro-1,6-naphthyridinyl, 1,2,3,4-tetrahydro-2,6-naphthyridinyl, 1,2,3,4-tetrahydro-3,6-naphthyridinyl, or 1,2,3,4-tetrahydro-4,6-naphthyridinyl, or 2,3-dihydrobenzo[d]oxazolyl, wherein any saturated carbon of Ring A is optionally substituted with oxo (═O) and wherein Ring A is optionally substituted with 1-6 R¹ substituents.

In some embodiments, Ring A is a partially unsaturated bicyclic heterocyclic ring selected from 3,4-dihydroquinolin-2(1H)-onyl, indolin-2-onyl, 3,4-dihydroisoquinolin-1(2H)-onyl, isoindolin-1-onyl, or benzo[d]oxazol-2(3H)-onyl; wherein Ring A is optionally substituted with 1-6 R¹ substituents.

In some embodiments, Ring A is phenyl, wherein Ring A is optionally substituted with 1-6 R¹ substituents. In some embodiments, Ring A is naphthyl, wherein Ring A is optionally substituted with 1-6 R¹ substituents. In some embodiments, Ring A is quinolinyl, wherein Ring A is optionally substituted with 1-6 R¹ substituents. In some embodiments, Ring A is isoquinolinyl, wherein Ring A is optionally substituted with 1-6 R¹ substituents. In some embodiments, Ring A is cinnolinyl, wherein Ring A is optionally substituted with 1-6 R¹ substituents. In some embodiments, Ring A is phthalazinyl, wherein Ring A is optionally substituted with 1-6 R¹ substituents. In some embodiments, Ring A is quinazolinyl, wherein Ring A is optionally substituted with 1-6 R¹ substituents. In some embodiments, Ring A is quinoxalinyl, wherein Ring A is optionally substituted with 1-6 R¹ substituents. In some embodiments, Ring A is 1,8-naphthyridinyl, wherein Ring A is optionally substituted with 1-6 R¹substituents. In some embodiments, Ring A is 1,7-naphthyridinyl, wherein Ring A is optionally substituted with 1-6 R¹ substituents. In some embodiments, Ring A is 1,6-naphthyridinyl, wherein Ring A is optionally substituted with 1-6 R¹ substituents. In some embodiments, Ring A is 1,5-naphthyridinyl, wherein Ring A is optionally substituted with 1-6 R¹ substituents.

In some embodiments, Ring A is indolinyl, wherein any saturated carbon of Ring A is optionally substituted with oxo (═O) and wherein Ring A is optionally substituted with 1-6 R¹ substituents. In some embodiments, Ring A is isoindolinyl, wherein any saturated carbon of Ring A is optionally substituted with oxo (═O) and wherein Ring A is optionally substituted with 1-6 R¹ substituents. In some embodiments, wherein any saturated carbon of Ring A is optionally substituted with oxo (═O) and Ring A is 1,2-dihydroisoquinolinyl, wherein Ring A is optionally substituted with 1-6 R¹ substituents. In some embodiments, wherein any saturated carbon of Ring A is optionally substituted with oxo (═O) and Ring A is 1,2,3,4-tetrahydroquinolinyl, wherein Ring A is optionally substituted with 1-6 R¹ substituents. In some embodiments, Ring A is 1,2,3,4-tetrahydroisoquinolinyl, wherein any saturated carbon of Ring A is optionally substituted with oxo (═O) and wherein Ring A is optionally substituted with 1-6 R¹ substituents. In some embodiments, Ring A is 1,2,3,4-tetrahydroquinoxalinyl, wherein any saturated carbon of Ring A is optionally substituted with oxo (═O) and wherein Ring A is optionally substituted with 1-6 R¹ substituents. In some embodiments, Ring A is 1,2,3,4-tetrahydro-1,8-naphthyridine, wherein any saturated carbon of Ring A is optionally substituted with oxo (═O) and wherein Ring A is optionally substituted with 1-6 R¹ substituents. In some embodiments, Ring A is 5,6,7,8-tetrahydro-1,7-naphthyridinyl, wherein any saturated carbon of Ring A is optionally substituted with oxo (═O) and wherein Ring A is optionally substituted with 1-6 R¹ substituents. In some embodiments, Ring A is 5,6,7,8-tetrahydro-1,6-naphthyridinyl, wherein any saturated carbon of Ring A is optionally substituted with oxo (═O) and wherein Ring A is optionally substituted with 1-6 R¹ substituents. In some embodiments, Ring A is 5,6,7,8-tetrahydro-1,5-naphthyridinyl, wherein any saturated carbon of Ring A is optionally substituted with oxo (═O) and wherein Ring A is optionally substituted with 1-6 R¹ substituents. In some embodiments, Ring A is 1,2,3,4-tetrahydro-1,6-naphthyridinyl, wherein any saturated carbon of Ring A is optionally substituted with oxo (═O) and wherein Ring A is optionally substituted with 1-6 R¹ substituents. In some embodiments, Ring A is 1,2,3,4-tetrahydro-2,6-naphthyridinyl, wherein any saturated carbon of Ring A is optionally substituted with oxo (═O) and wherein Ring A is optionally substituted with 1-6 R¹ substituents. In some embodiments, Ring A is 1,2,3,4-tetrahydro-3,6-naphthyridinyl, wherein any saturated carbon of Ring A is optionally substituted with oxo (═O) and wherein Ring A is optionally substituted with 1-6 R¹ substituents. In some embodiments, Ring A is 1,2,3,4-tetrahydro-4,6-naphthyridinyl, wherein any saturated carbon of Ring A is optionally substituted with oxo (═O) and wherein Ring A is optionally substituted with 1-6 R¹ substituents. In some embodiments, Ring A is 2,3-dihydrobenzo[d]oxazole, wherein any saturated carbon of Ring A is optionally substituted with oxo (═O) and wherein Ring A is optionally substituted with 1-6 R¹ substituents.

In some embodiments, Ring A is 3,4-dihydroquinolin-2(1H)-onyl, wherein Ring A is optionally substituted with 1-6 R¹ substituents. In some embodiments, Ring A is indolin-2-onyl, wherein Ring A is optionally substituted with 1-6 R¹ substituents. In some embodiments, Ring A is 3,4-dihydroisoquinolin-1(2H)-onyl, wherein Ring A is optionally substituted with 1-6 R¹ substituents. In some embodiments, Ring A is isoindolin-1-onyl, wherein Ring A is optionally substituted with 1-6 R¹ substituents. In some embodiments, Ring A is benzo[d]oxazol-2(31)-onyl, wherein Ring A is optionally substituted with 1-6 R¹ substituents.

In some embodiments, Ring A is phenyl, wherein Ring A is optionally substituted with 1-6 R¹ substituents.

In some embodiments, Ring A is tetrahydroisoquinolinyl, wherein Ring A is optionally substituted with 1-6 R¹ substituents.

In some embodiments, each R¹ is independently selected from the group consisting of —F, —C₁, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl. In some embodiments, each R¹ is independently selected from the group consisting of —F, —Cl, —Br, —I, —OH and —C₁-C₆ alkyl. In some embodiments, R¹ is —F. In some embodiments, R¹ is —C₁. In some embodiments, R¹ is —Br. In some embodiments, R¹ is —I. In some embodiments, R¹ is —OH. In some embodiments, R¹ is —CF₃. In some embodiments, R¹ is —CN. In some embodiments, R¹ is —CO₂H. In some embodiments, R¹ is —C(═O)OCH₃. In some embodiments, R¹ is —C(═O)NH₂. In some embodiments, R¹ is —CH(═N)OH. In some embodiments, R¹ is —NH₂. In some embodiments, R¹ is —NHCH₃. In some embodiments, R¹ is —N(CH₃)₂. In some embodiments, R¹ is —NO₂. In some embodiments, R¹ is —NHC(═O)CH₃. In some embodiments, R¹ is —NHCO₂H. In some embodiments, R¹ is —NHC(═O)OCH₃. In some embodiments, R¹ is —OC(═O)NH₂. In some embodiments, R¹ is and substituted or unsubstituted —C₁-C₆ alkyl. In some embodiments, R¹ is —CH₃. In some embodiments, R¹ is —CH₂CH₃. In some embodiments, R¹ is —CH(CH₃)₂.

In some embodiments, X is absent, —O—, —NH—, —S—, —S(═O)(═NH)—, ═S(═NH)(═NH), —CH═CH—C(═O)—, or —CH₂CH₂—C(═O)—. In some embodiments, X is a bond. In some embodiments, X is —O— or —NH—. In some embodiments, X is —S(═O)(═NH)— or ═S(═NH)(═NH). In some embodiments, X is absent. In some embodiments, X is —O—. In some embodiments, X is —NH—. In some embodiments, X is —S—. In some embodiments, X is —S(═O)(═NH)—. In some embodiments, X is ═S(═NH)(═NH). In some embodiments, X is —CH₂CH₂—C(═O)—. In some embodiments, X is or —CH₂CH₂—C(═O)—.

In some embodiments, Ring B is piperidinyl, pyrrolidinyl, or azetidinyl, wherein Ring B is optionally substituted with one or more R² substituents; and wherein any saturated carbon of Ring B is optionally substituted with oxo (═O). In some embodiments, Ring B is piperidinyl, pyrrolidinyl, or azetidinyl, wherein Ring B is optionally substituted with one or more R^{z}substituents. In some embodiments, Ring B is piperidinyl, wherein Ring B is optionally substituted with one or more R² substituents. In some embodiments, Ring B is unsubstituted piperidinyl. In some embodiments, Ring B is pyrrolidinyl, wherein Ring B is optionally substituted with one or more R² substituents; and wherein any saturated carbon of Ring B is optionally substituted with oxo (═O). In some embodiments, Ring B is pyrrolidinyl, wherein Ring B is optionally substituted with one or more R² substituents. In some embodiments, Ring B is azetidinyl, wherein Ring B is optionally substituted with one or more R² substituents. In some embodiments, Ring B is piperazinyl, wherein Ring B is optionally substituted with one or more R² substituents. In some embodiments, Ring B is 1,2,3,6-tetrahydropyridinyl, wherein Ring B is optionally substituted with one or more R² substituents. In some embodiments, Ring B is 3,6-diazabicyclo[3.1.1]heptanyl, wherein Ring B is optionally substituted with one or more R² substituents. In some embodiments, Ring B is cyclobutyl, cyclopentyl, or cyclohexyl, wherein Ring B is optionally substituted with one or more R² substituents. In some embodiments, Ring B is cyclobutyl, wherein Ring B is optionally substituted with one or more R² substituents. In some embodiments, Ring B is cyclopentyl, wherein Ring B is optionally substituted with one or more R² substituents. In some embodiments, Ring B is cyclohexyl, wherein Ring B is optionally substituted with one or more R² substituents.

In some embodiments, Ring B is absent and Ring A is a partially unsaturated bicyclic heterocyclic ring. In some embodiments, Ring B is absent and Ring A is indolinyl, wherein Ring A is optionally substituted with 1-6 R¹ substituents. In some embodiments, Ring B is absent and Ring A is isoindolinyl, wherein Ring A is optionally substituted with 1-6 R¹ substituents. In some embodiments, Ring B is absent and Ring A is 1,2-dihydroisoquinolinyl, wherein Ring A is optionally substituted with 1-6 R¹ substituents. In some embodiments, Ring B is absent and Ring A is 1,2,3,4-tetrahydroquinolinyl, wherein Ring A is optionally substituted with 1-6 R¹ substituents. In some embodiments, Ring B is absent and Ring A is 1,2,3,4-tetrahydroisoquinolinyl, wherein Ring A is optionally substituted with 1-6 R¹ substituents. In some embodiments, Ring B is absent and Ring A is 1,2,3,4-tetrahydroquinoxalinyl, wherein Ring A is optionally substituted with 1-6 R¹ substituents. In some embodiments, Ring B is absent and Ring A is 1,2,3,4-tetrahydro-1,8-naphthyridine, wherein Ring A is optionally substituted with 1-6 R¹ substituents. In some embodiments, Ring B is absent and Ring A is 5,6,7,8-tetrahydro-1,7-naphthyridinyl, wherein Ring A is optionally substituted with 1-6 R¹ substituents. In some embodiments, Ring B is absent and Ring A is 5,6,7,8-tetrahydro-1,6-naphthyridinyl, wherein Ring A is optionally substituted with 1-6 R¹ substituents. In some embodiments, Ring B is absent and Ring A is 5,6,7,8-tetrahydro-1,5-naphthyridinyl, wherein Ring A is optionally substituted with 1-6 R¹ substituents. In some embodiments, Ring B is absent and Ring A is 1,2,3,4-tetrahydro-1,6-naphthyridinyl, wherein Ring A is optionally substituted with 1-6 R¹ substituents. In some embodiments, Ring B is absent and Ring A is 1,2,3,4-tetrahydro-2,6-naphthyridinyl, wherein Ring A is optionally substituted with 1-6 R¹ substituents. In some embodiments, Ring B is absent and Ring A is 1,2,3,4-tetrahydro-3,6-naphthyridinyl, wherein Ring A is optionally substituted with 1-6 R¹ substituents. In some embodiments, Ring B is absent and Ring A is 1,2,3,4-tetrahydro-4,6-naphthyridinyl, wherein Ring A is optionally substituted with 1-6 R¹ substituents. In some embodiments, Ring B is absent and Ring A is 2,3-dihydrobenzo[d]oxazolyl, wherein Ring A is optionally substituted with 1-6 R¹ substituents.

In some embodiments, Ring B is absent and Ring A is 3,4-dihydroquinolin-2(1H)-onyl, wherein Ring A is optionally substituted with 1-6 R¹ substituents. In some embodiments, Ring B is absent and Ring A is indolin-2-onyl, wherein Ring A is optionally substituted with 1-6 R¹ substituents. In some embodiments, Ring B is absent and Ring A is 3,4-dihydroisoquinolin-1(2H)-onyl, wherein Ring A is optionally substituted with 1-6 R¹ substituents. In some embodiments, Ring B is absent and Ring A is isoindolin-1-onyl, wherein Ring A is optionally substituted with 1-6 R¹ substituents. In some embodiments, Ring B is absent and Ring A is benzo[d]oxazol-2(3H)-onyl, wherein Ring A is optionally substituted with 1-6 R¹ substituents.

In some embodiments, each R² is independently selected from the group consisting of —F, —CF₃, —CN, and substituted or unsubstituted —C₁-C₆ alkyl. In some embodiments, each R² is independently selected from the group consisting of —F, —CF₃, and —CH₃. In some embodiments, R² is —F. In some embodiments, R² is —CF₃. In some embodiments, R² is —CN. In some embodiments, R² is substituted or unsubstituted —C₁-C₆ alkyl. In some embodiments, R² is —F or substituted or unsubstituted —C₁-C₆ alkyl. In some embodiments, R² is —F. In some embodiments, R² is —CH₃. some embodiments, R¹ is —CH₂NH₂. In some embodiments, two R² variables on the same carbon are both —F. In some embodiments, two R² variables on the same carbon are both —CH₃.

In some embodiments, two R² variables on the same carbon atom come together to form an unsubstituted C₃-C₆ cycloalkyl ring. In some embodiments, two R² variables on the same carbon atom come together to form an unsubstituted cyclopropyl ring. In some embodiments, two R² variables on the same carbon atom come together to form an unsubstituted cyclobutyl ring. In some embodiments, two R² variables on the same carbon atom come together to form an unsubstituted cyclopentyl ring. In some embodiments, two R² variables on the same carbon atom come together to form an unsubstituted cyclohexyl ring.

In some embodiments, two R² variables on adjacent carbon atoms come together to form an unsubstituted C₃-C₅ cycloalkyl ring. In some embodiments, two R² variables on adjacent carbon atoms come together to form an unsubstituted cyclopropyl ring. In some embodiments, two R² variables on adjacent carbon atoms come together to form an unsubstituted cyclobutyl ring. In some embodiments, two R² variables on adjacent carbon atoms come together to form an unsubstituted cyclopentyl ring. In some embodiments, two R² variables on adjacent carbon atoms come together to form an unsubstituted cyclohexyl ring.

In some embodiments, Ring A is

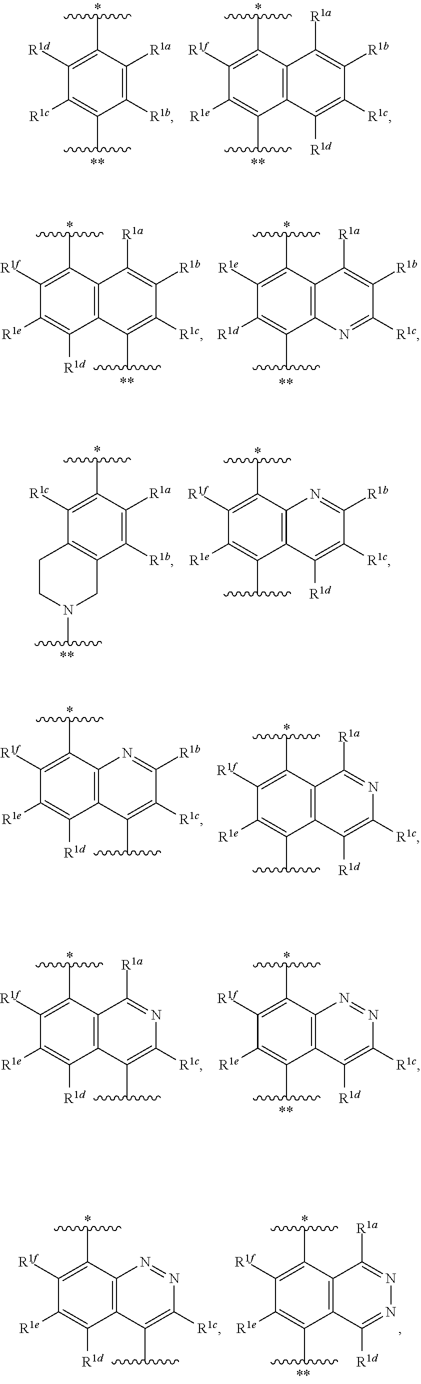

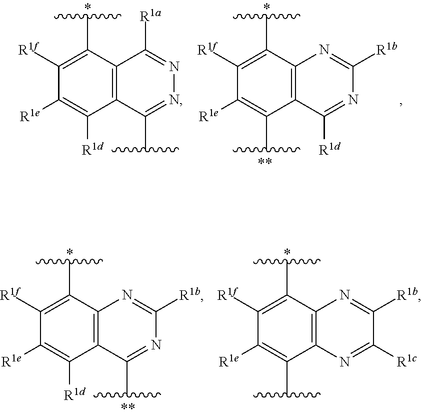

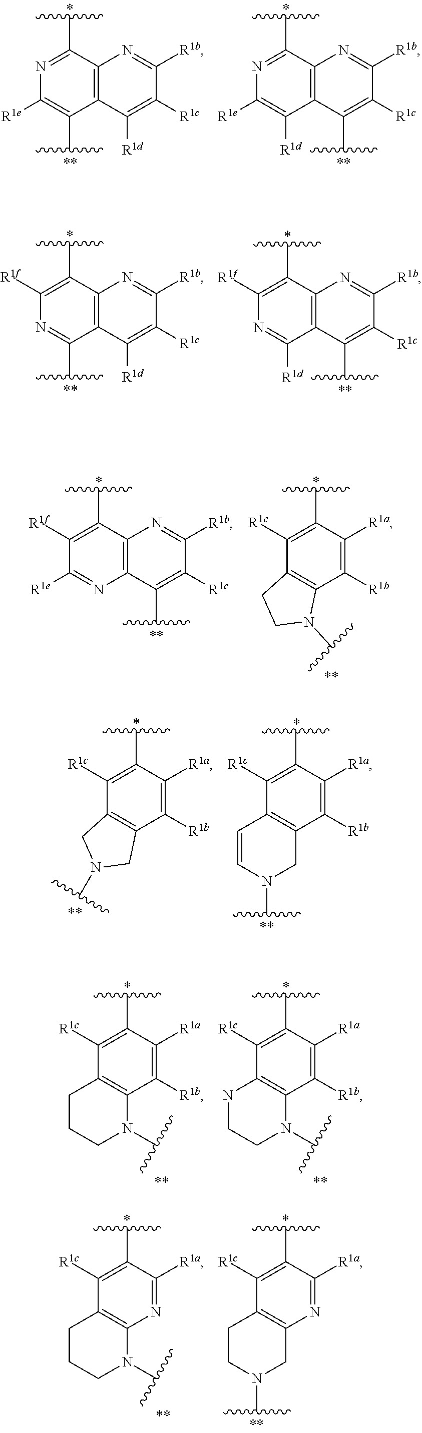

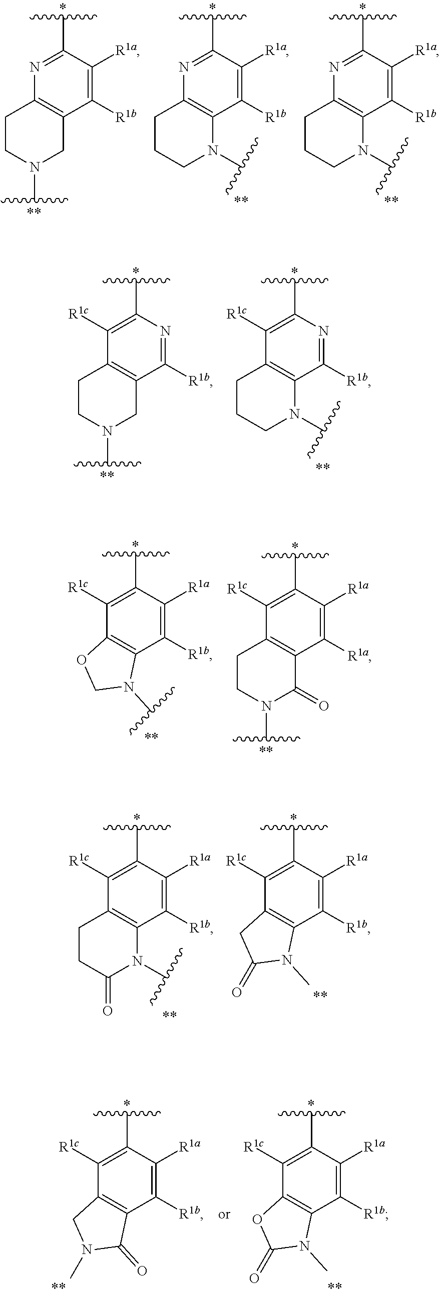

wherein * denotes the attachment point of Ring A to the pyrazole moiety of Formula (A) or Formula (I) and ** denotes the attachment point of Ring A to X.

In some embodiments, Ring A is

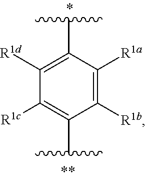

wherein * denotes the attachment point of Ring A to the pyrazole moiety of Formula (A) or Formula (I) and ** denotes the attachment point of Ring A to X. In some embodiments, Ring A is

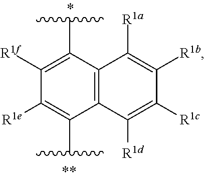

wherein * denotes the attachment point of Ring A to the pyrazole moiety of Formula (A) or Formula (I) and ** denotes the attachment point of Ring A to X. In some embodiments, Ring A is

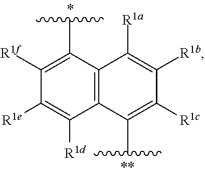

wherein * denotes the attachment point of Ring A to the pyrazole moiety of Formula (A) or Formula (I) and ** denotes the attachment point of Ring A to X. In some embodiments, Ring A is

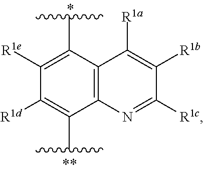

wherein * denotes the attachment point of Ring A to the pyrazole moiety of Formula (A) or Formula (I) and ** denotes the attachment point of Ring A to X. In some embodiments, Ring A is

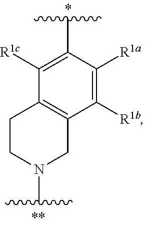

wherein * denotes the attachment point of Ring A to the pyrazole moiety of Formula (A) or Formula (I) and ** denotes the attachment point of Ring A to X.

In some embodiments, —X-Ring B— is a bond,

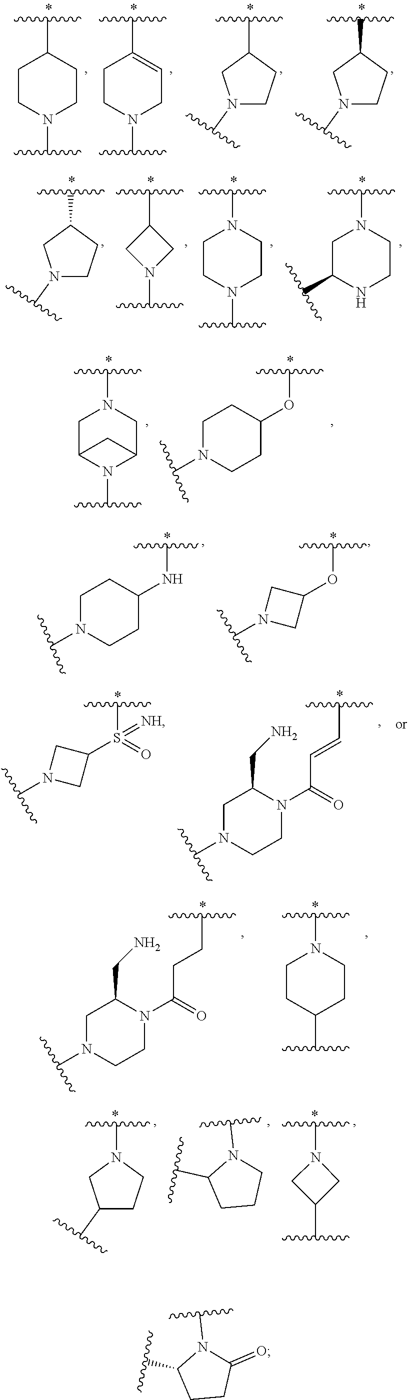

wherein * denotes the attachment point of —X-Ring B— to Ring A. In some embodiments, —X-Ring B— is a bond,

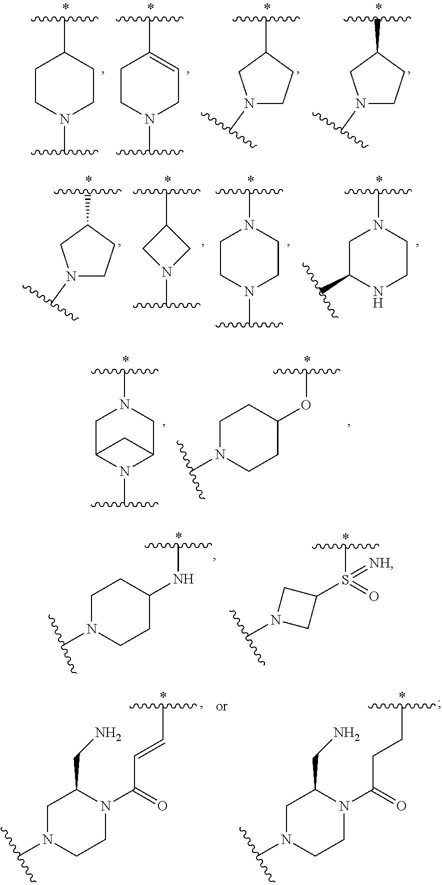

wherein* denotes the attachment point of —X-Ring B— to Ring A. In some embodiments, —X-Ring B— is

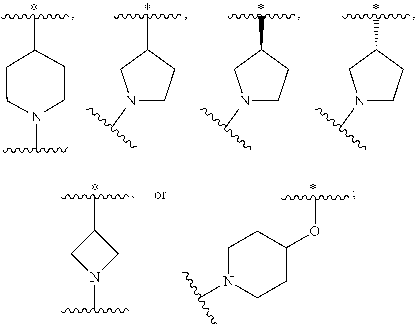 [SMILES: *C1CCN(*)CC1 ; *C1CCN(*)C1 ; *[C@H]1CCN(*)C1 ; *[C@@H]1CCN(*)C1 ; *C1CN(*)C1 ; *OC1CCN(*)CC1]

wherein * denotes the attachment point of —X-Ring B— to Ring A.

In some embodiments, Ring A is

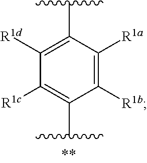

wherein ** denotes the attachment point of Ring A to X; and —X-Ring B— is

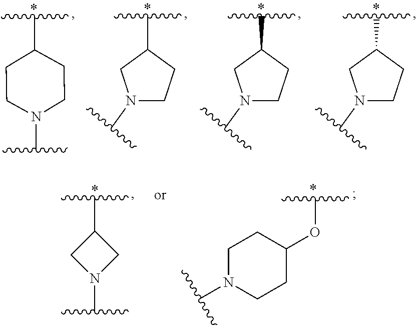 [SMILES: *C1CCN(*)CC1 ; *C1CCN(*)C1 ; *[C@H]1CCN(*)C1 ; *[C@@H]1CCN(*)C1 ; *C1CN(*)C1 ; *OC1CCN(*)CC1]

wherein * denotes the attachment point of —X-Ring B— to Ring A.

In some embodiments, —X-Ring B— is a bond. In some embodiments, —X-Ring B— is

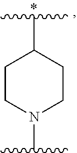 [SMILES: *C1CCN(*)CC1]

wherein * denotes the attachment point of —X-Ring B— to Ring A, and wherein Ring B is optionally substituted with one or more R² substituents. In some embodiments, —X-Ring B— is

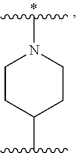 [SMILES: *C1CCN(*)CC1]

wherein * denotes the attachment point of —X-Ring B— to Ring A, and wherein Ring B is optionally substituted with one or more R² substituents. In some embodiments, —X-Ring B— is

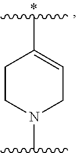 [SMILES: *C1=CCN(*)CC1]

wherein * denotes the attachment point of —X-Ring B— to Ring A, and wherein Ring B is optionally substituted with one or more R² substituents. In some embodiments, —X-Ring B— is

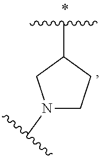 [SMILES: *C1CCN(*)C1]

wherein * denotes the attachment point of —X-Ring B— to Ring A, and wherein Ring B is optionally substituted with one or more R² substituents. In some embodiments, —X-Ring B— is

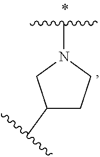 [SMILES: *C1CCN(*)C1]

wherein * denotes the attachment point of —X-Ring B— to Ring A, and wherein Ring B is optionally substituted with one or more R² substituents; and wherein any saturated carbon of Ring B is optionally substituted with oxo (═O). In some embodiments, —X-Ring B— is

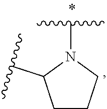 [SMILES: *C1CCCN1*]

wherein * denotes the attachment point of —X-Ring B— to Ring A, and wherein Ring B is optionally substituted with one or more R² substituents; and wherein any saturated carbon of Ring B is optionally substituted with oxo (═O). In some embodiments, —X-Ring B— is

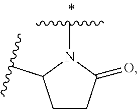 [SMILES: *C1CCC(=O)N1*]

wherein * denotes the attachment point of —X-Ring B— to Ring A, and wherein Ring B is optionally substituted with one or more R² substituents. In some embodiments, —X-Ring B— is

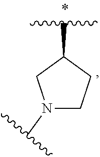 [SMILES: *[C@H]1CCN(*)C1]

wherein * denotes the attachment point of —X-Ring B— to Ring A, and wherein Ring B is optionally substituted with one or more R² substituents. In some embodiments, —X-Ring B— is

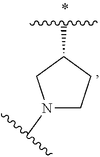 [SMILES: *[C@@H]1CCN(*)C1]

wherein * denotes the attachment point of —X-Ring B— to Ring A, and wherein Ring B is optionally substituted with one or more R² substituents. In some embodiments, —X-Ring B— is

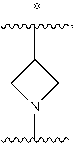 [SMILES: *C1CN(*)C1]

wherein * denotes the attachment point of —X-Ring B— to Ring A, and wherein Ring B is optionally substituted with one or more R² substituents. In some embodiments, —X-Ring B— is

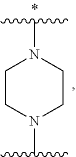 [SMILES: *N1CCN(*)CC1]

wherein * denotes the attachment point of —X-Ring B— to Ring A, and wherein Ring B is optionally substituted with one or more R² substituents. In some embodiments, —X-Ring B— is

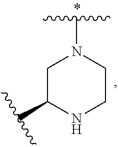 [SMILES: *[C@H]1CN(*)CCN1]

wherein * denotes the attachment point of —X-Ring B— to Ring A, and wherein Ring B is optionally substituted with one or more R² substituents. In some embodiments, —X-Ring B— is

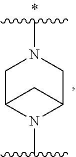 [SMILES: *N1CC2CC(C1)N2*]

wherein * denotes the attachment point of —X-Ring B— to Ring A, and wherein Ring B is optionally substituted with one or more R² substituents. In some embodiments, —X-Ring B— is

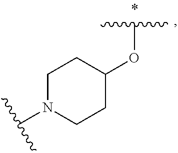 [SMILES: *OC1CCN(*)CC1]

wherein * denotes the attachment point of —X-Ring B— to Ring A, and wherein Ring B is optionally substituted with one or more R² substituents. In some embodiments, —X-Ring B— is

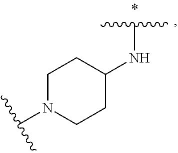 [SMILES: *NC1CCN(*)CC1]

wherein * denotes the attachment point of —X-Ring B— to Ring A, and wherein Ring B is optionally substituted with one or more R² substituents. In some embodiments, —X-Ring B— is

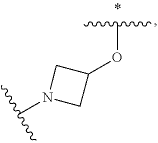 [SMILES: *OC1CN(*)C1]

wherein * denotes the attachment point of —X-Ring B— to Ring A, and wherein Ring B is optionally substituted with one or more R² substituents. In some embodiments, —X-Ring B— is

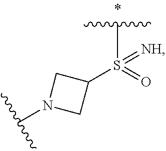 [SMILES: *N1CC(S(*)(=N)=O)C1]

wherein * denotes the attachment point of —X-Ring B— to Ring A, and wherein Ring B is optionally substituted with one or more R² substituents. In some embodiments, —X-Ring B— is

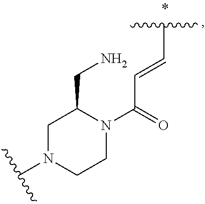 [SMILES: */C=C/C(=O)N1CCN(*)C[C@H]1CN]

wherein * denotes the attachment point of —X-Ring B— to Ring A, and wherein Ring B is optionally substituted with one or more R² substituents. In some embodiments, —X—Ring B— is

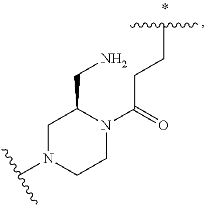 [SMILES: *CCC(=O)N1CCN(*)C[C@H]1CN]

wherein * denotes the attachment point of —X-Ring B— to Ring A, and wherein Ring B is optionally substituted with one or more R² substituents.

In some embodiments, Ring A-X-Ring B is:

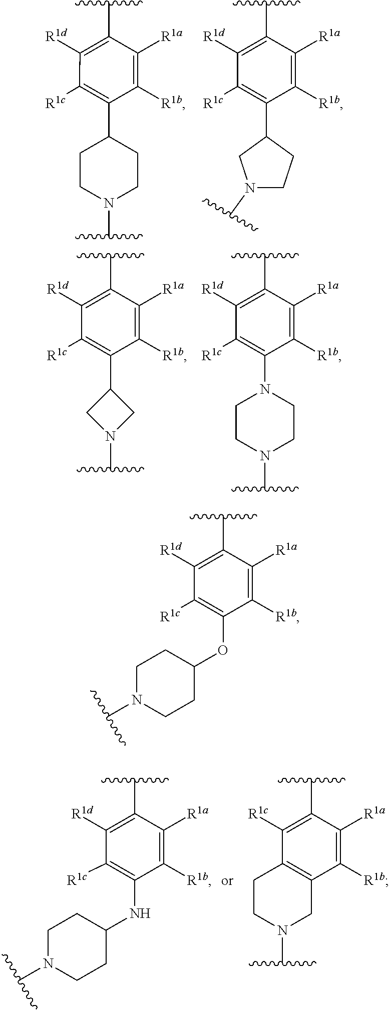

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, Ring A-X-Ring B is:

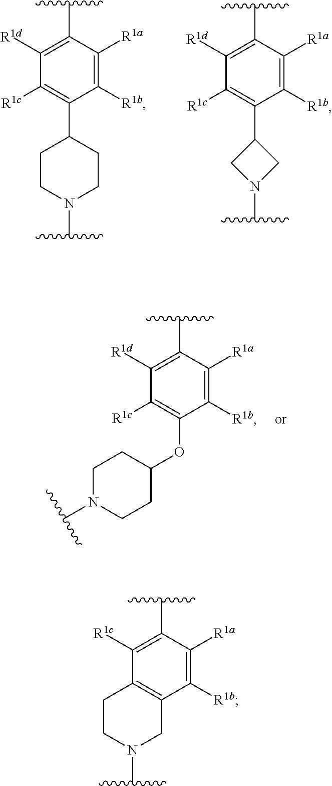

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)N—H₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, Ring A-X-Ring B is:

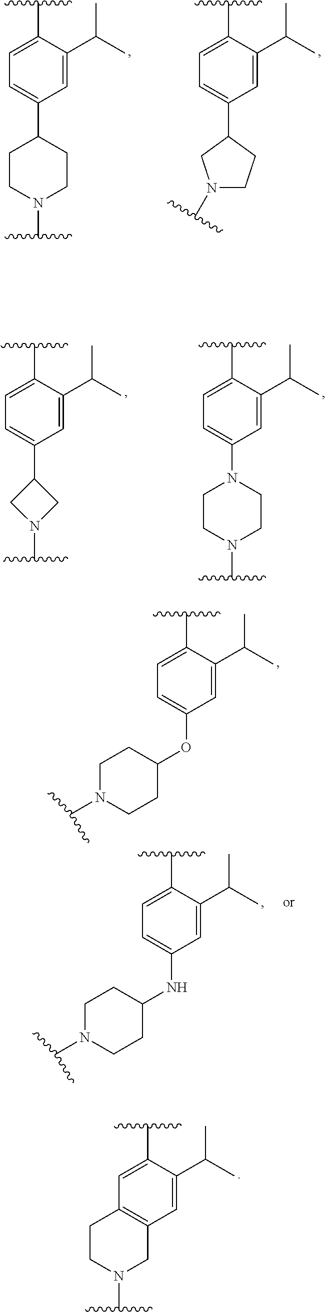

In some embodiments, Ring A-X-Ring B is:

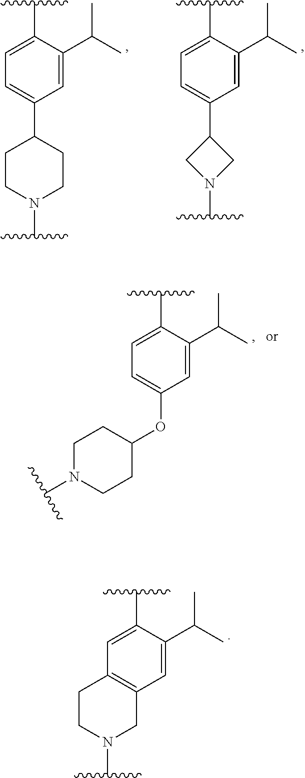 [SMILES: *c1ccc(C2CCN(*)CC2)cc1C(C)C ; *c1ccc(C2CN(*)C2)cc1C(C)C ; *c1ccc(OC2CCN(*)CC2)cc1C(C)C ; *c1cc2c(cc1C(C)C)CN(*)CC2]

In some embodiments, Ring A-X-Ring B is

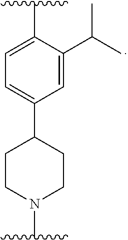 [SMILES: *c1ccc(C2CCN(*)CC2)cc1C(C)C]

In some embodiments, Ring A-X-Ring B is:

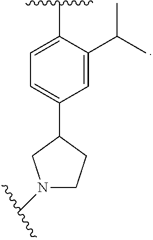 [SMILES: *c1ccc(C2CCN(*)C2)cc1C(C)C]

In some embodiments, Ring A-X-Ring B is:

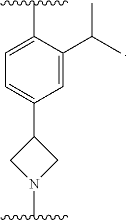 [SMILES: *c1ccc(C2CN(*)C2)cc1C(C)C]

In some embodiments, Ring A-X-Ring B is:

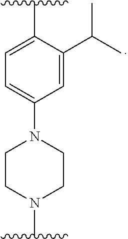 [SMILES: *c1ccc(N2CCN(*)CC2)cc1C(C)C]

In embodiments, Ring A-X-Ring B is:

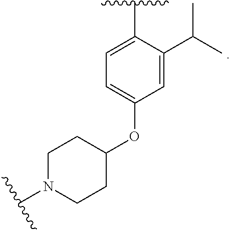 [SMILES: *c1ccc(OC2CCN(*)CC2)cc1C(C)C]

In some embodiments, Ring A-X-Ring B is

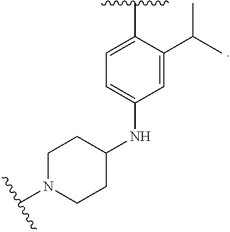 [SMILES: *c1ccc(NC2CCN(*)CC2)cc1C(C)C]

In some embodiments, Ring A-X-Ring B is:

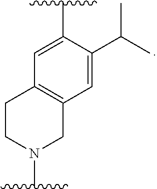 [SMILES: *c1cc2c(cc1C(C)C)CN(*)CC2]

In some embodiments, the compound has the structure of Formula (B):

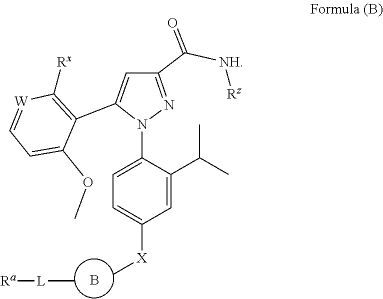

In some embodiments, the compound has the structure of Formula (C):

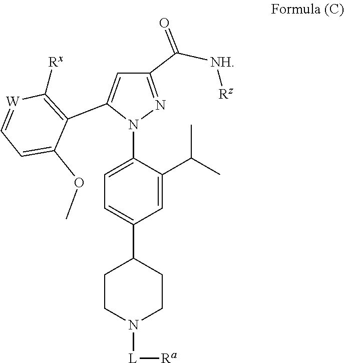

In some embodiments, the compound has the following structure:

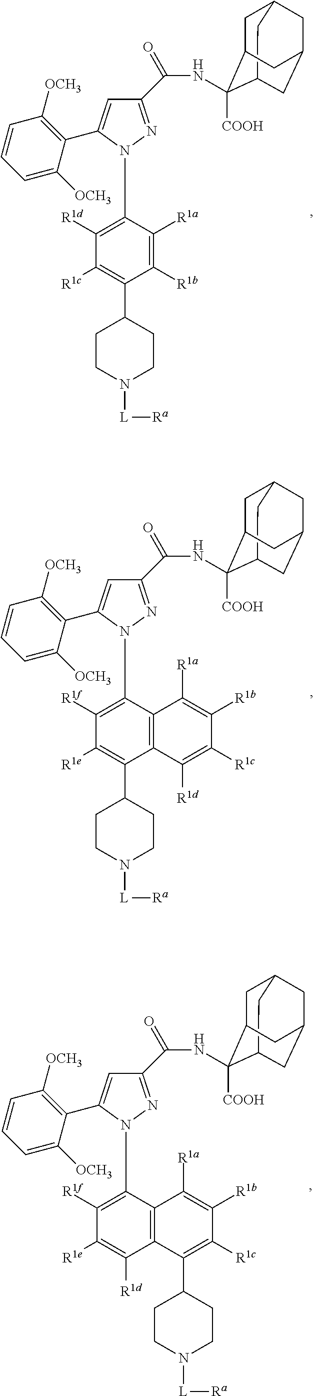

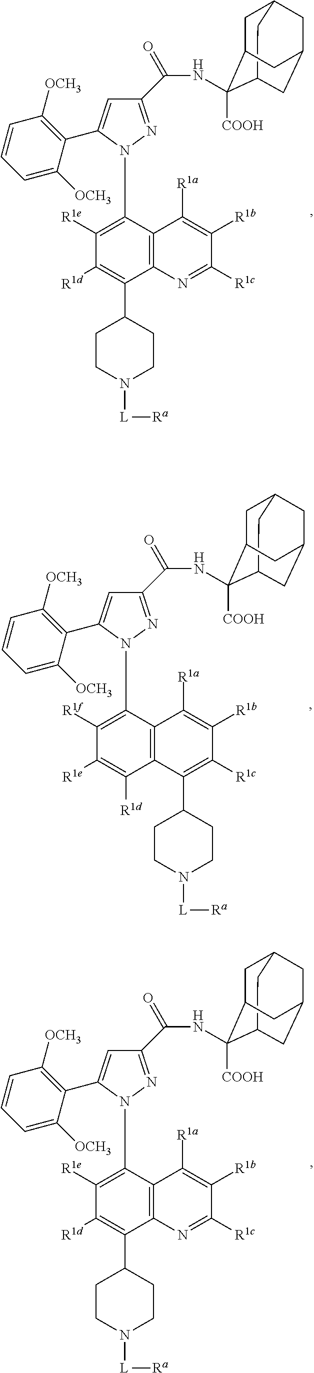

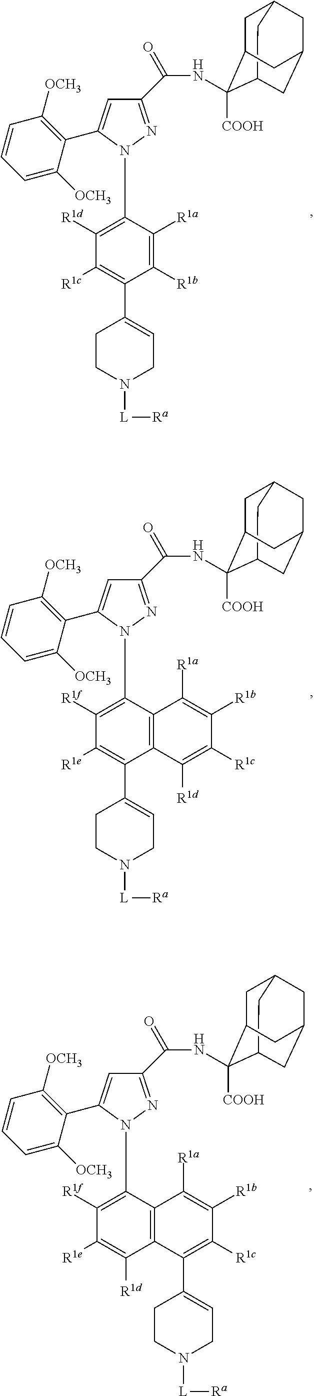

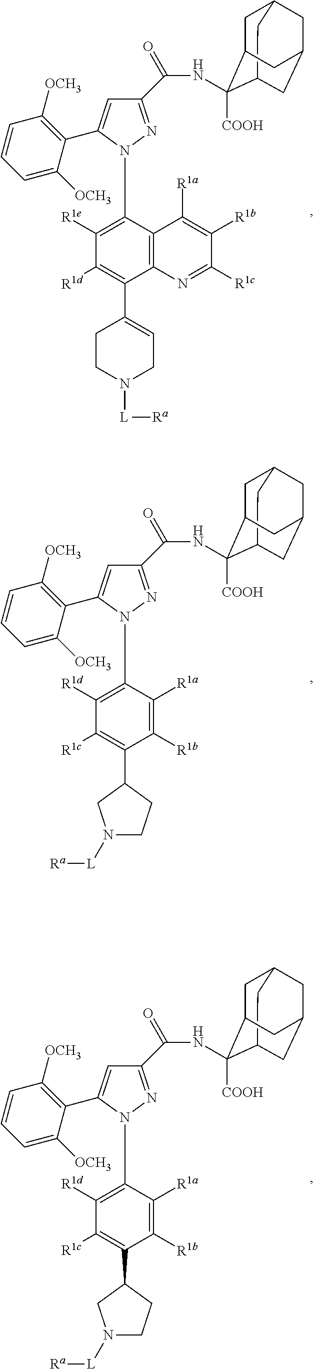

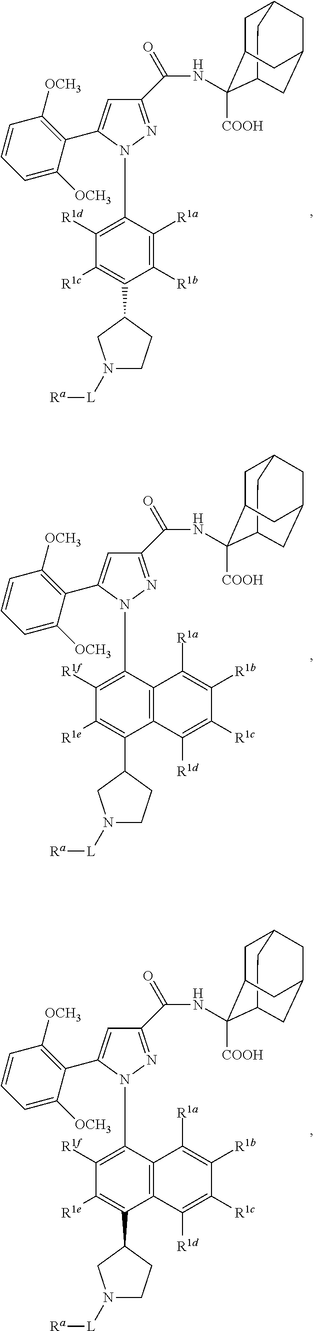

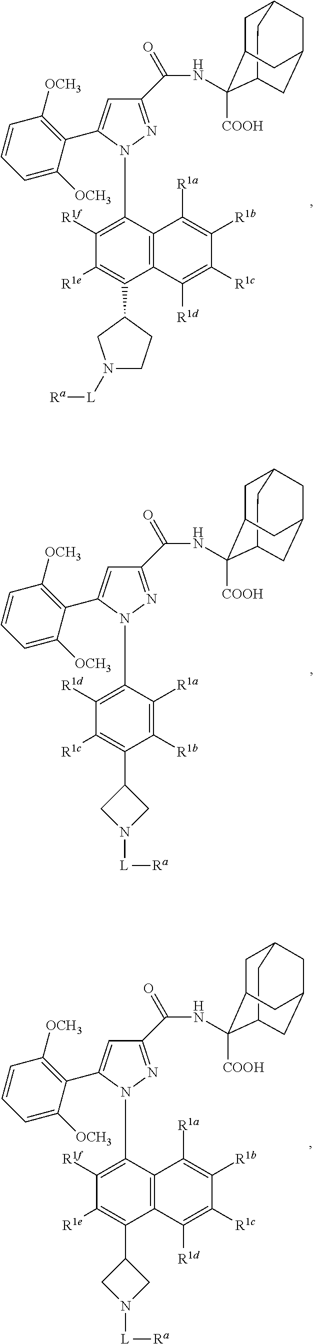

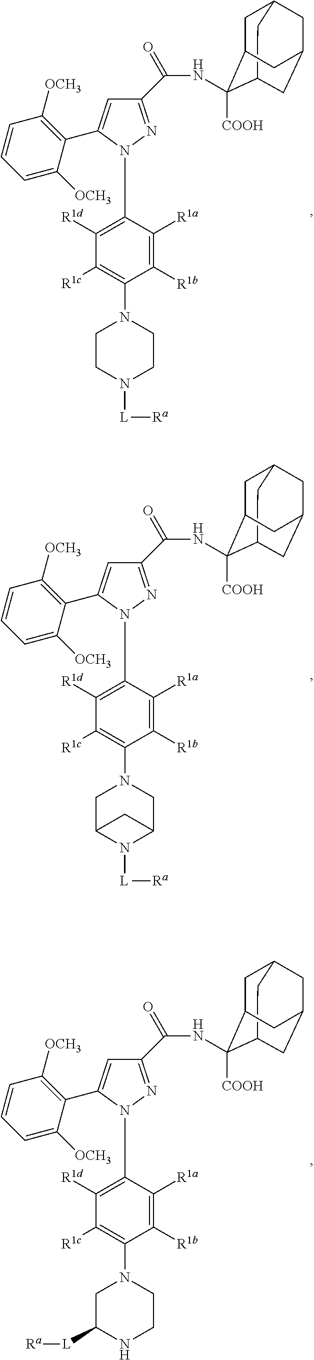

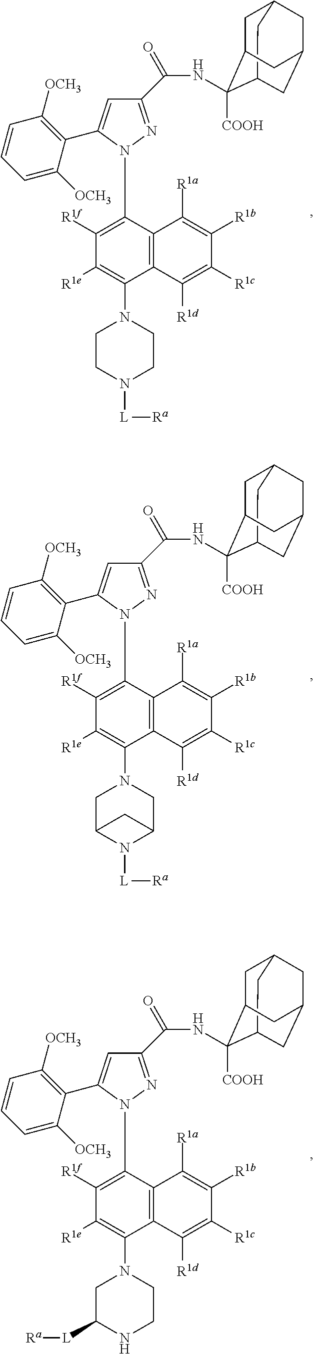

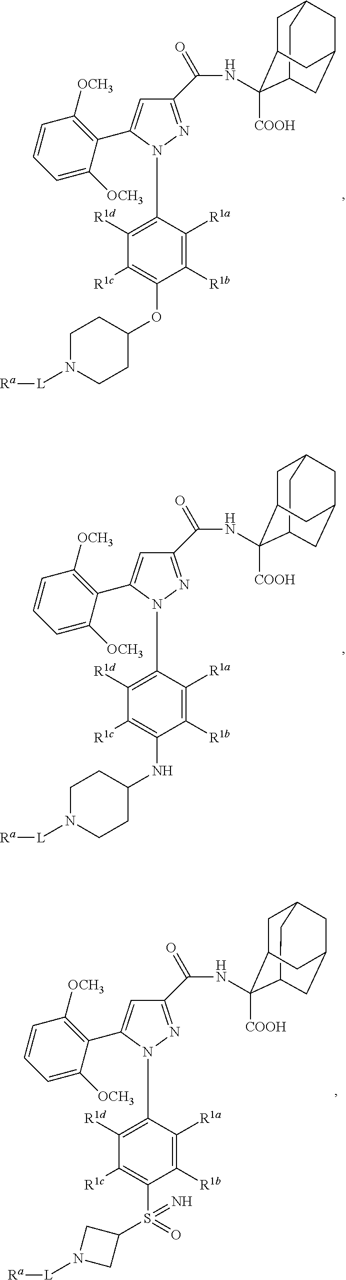

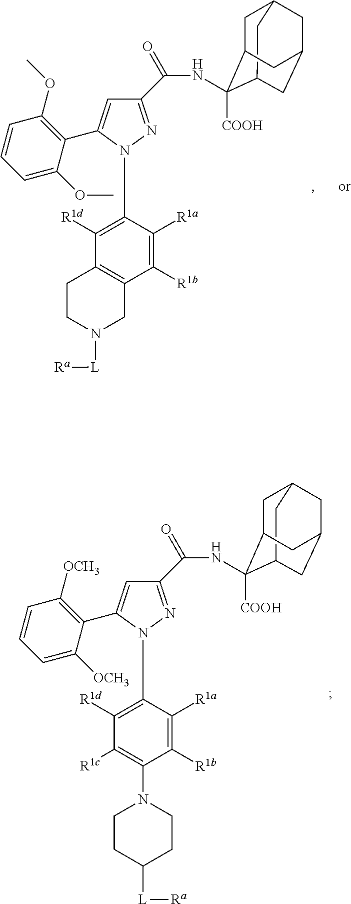

wherein R¹, R^{1b}, R^{1c}, and R^{1d} are each independently selected from the group consisting of hydrogen, —F, —Cl, —Br, -L, —OH, —CF₃, —CN, —CO₂H, —C(═O)OCH₃, —C(═O)NH₂, —CH(═N)OH, —NH₂, —NHCH, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the following structure:

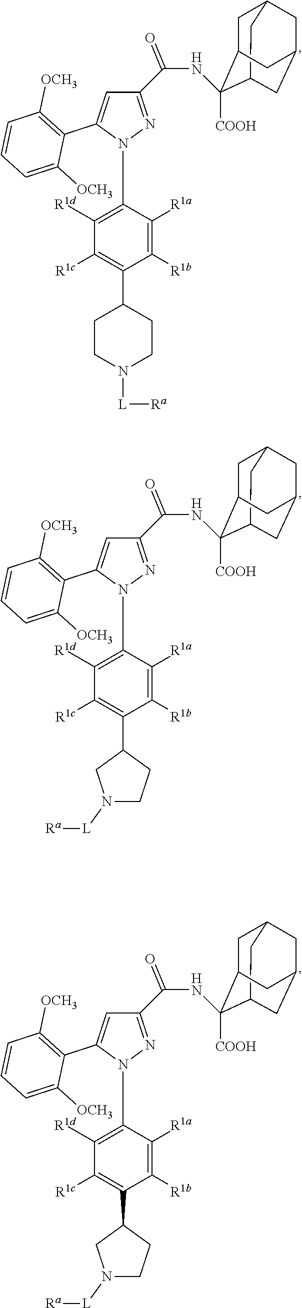

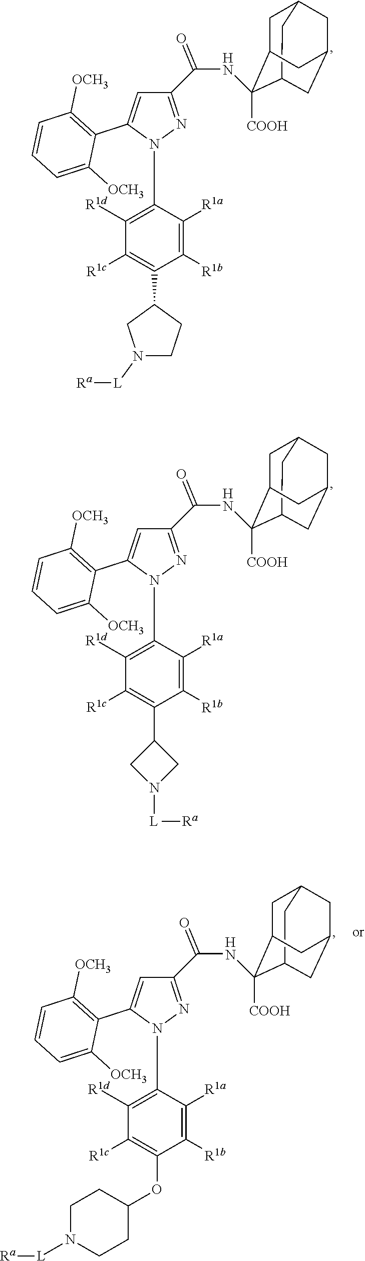

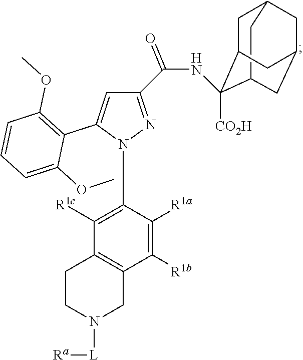

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the following structure:

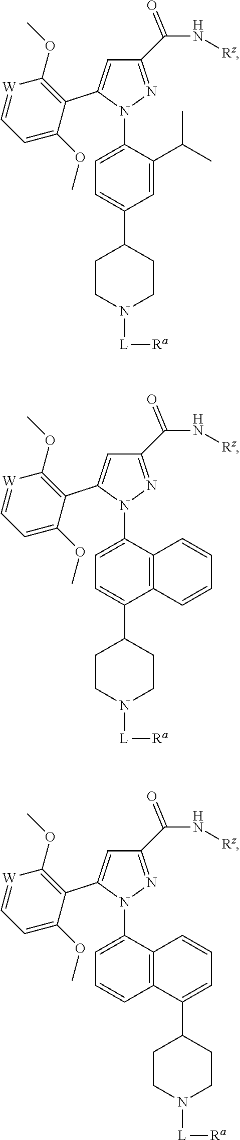

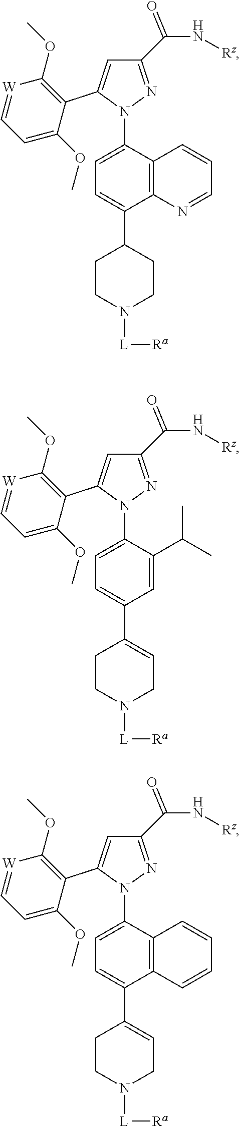

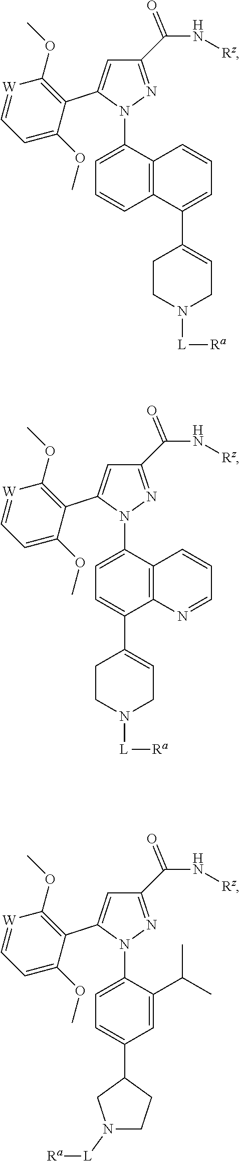

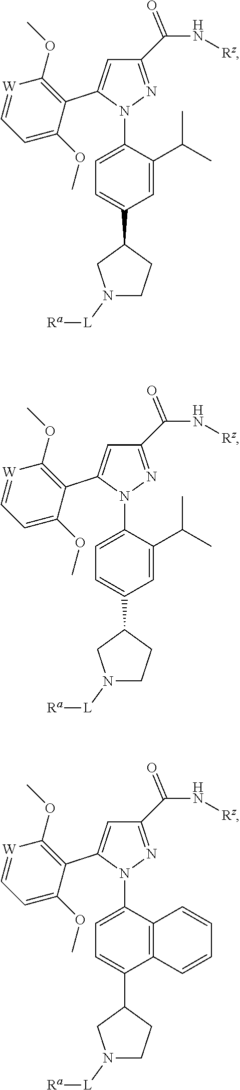

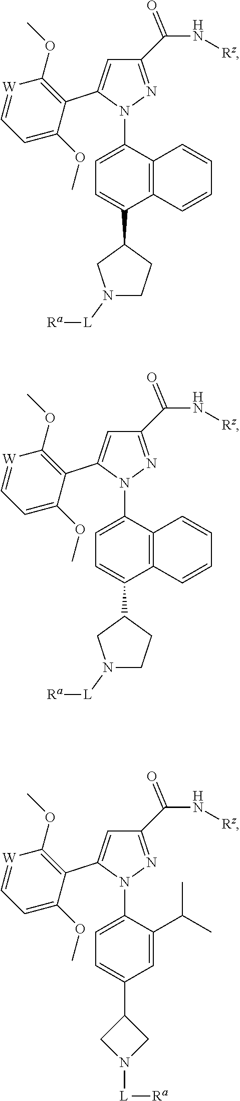

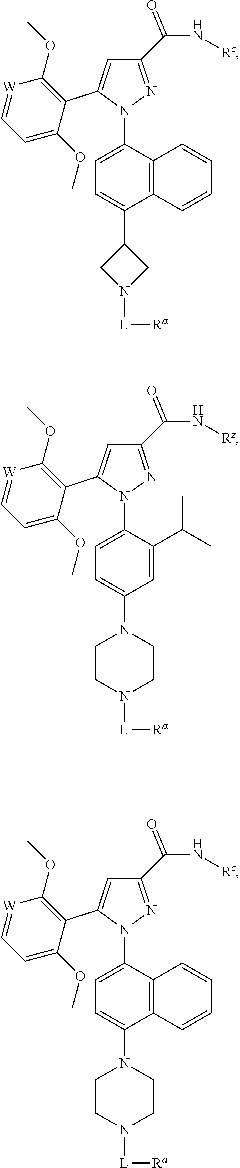

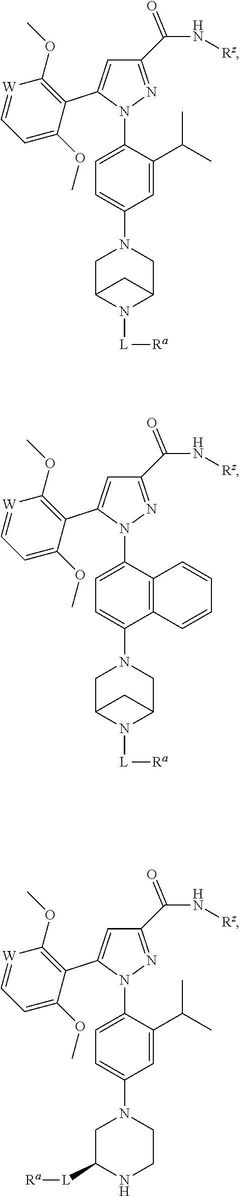

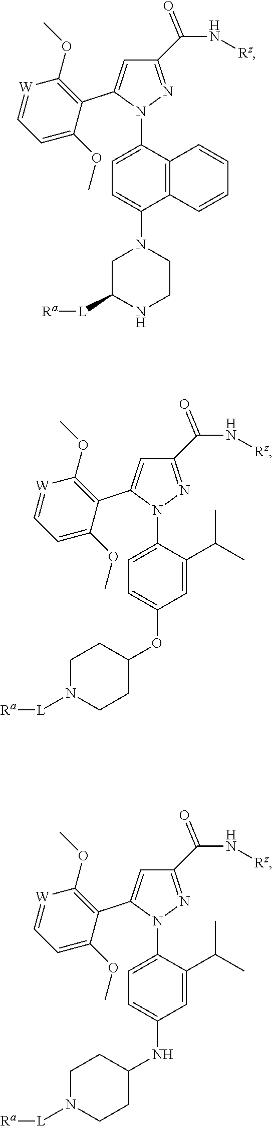

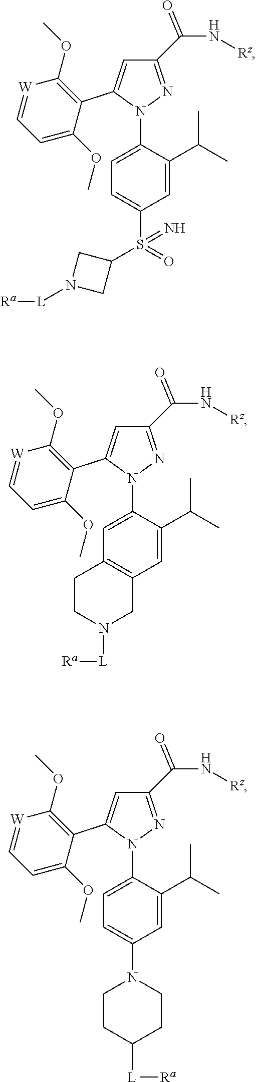

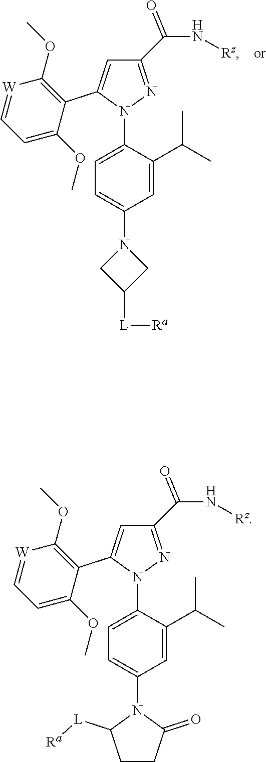

In some embodiments, the compound has the following structure:

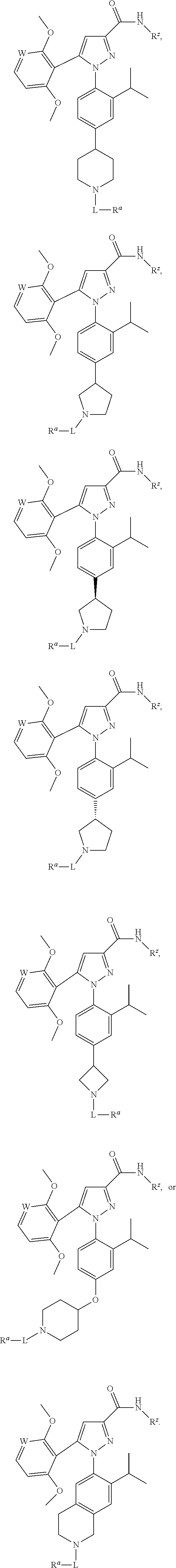

In some embodiments, the compound has the following structure:

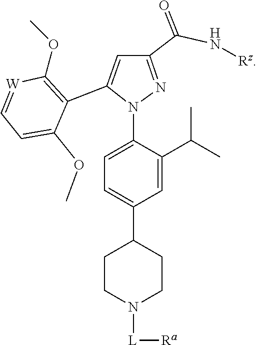

In some embodiments, the compound has the following structure:

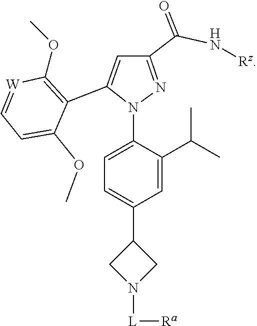

In some embodiments, the compound has the following structure:

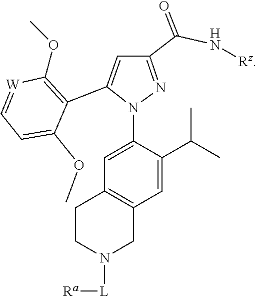

In some embodiments, the compound has the following structure:

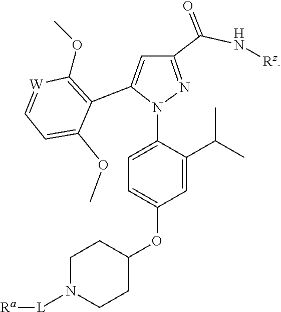

In some embodiments, W is —CH—. In some embodiments, W is N. In some embodiments, W is —C(F)—. In some embodiments, W is —C(OMe)-. In some embodiments, W is —C(OEt)-. In some embodiments, R^{z} is

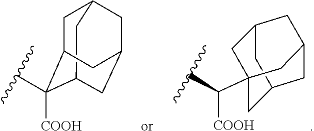 [SMILES: *C1(C(=O)O)C2CC3CC(C2)CC1C3 ; *[C@H](C(=O)O)C12CC3CC(CC(C3)C1)C2]

In some embodiments, the compound has the following structure:

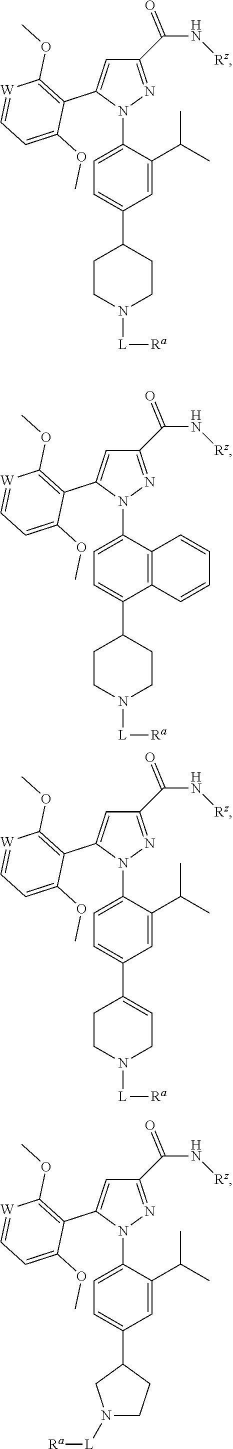

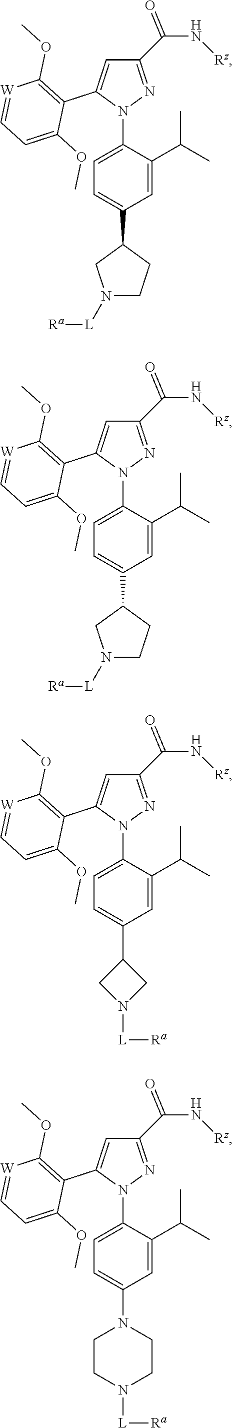

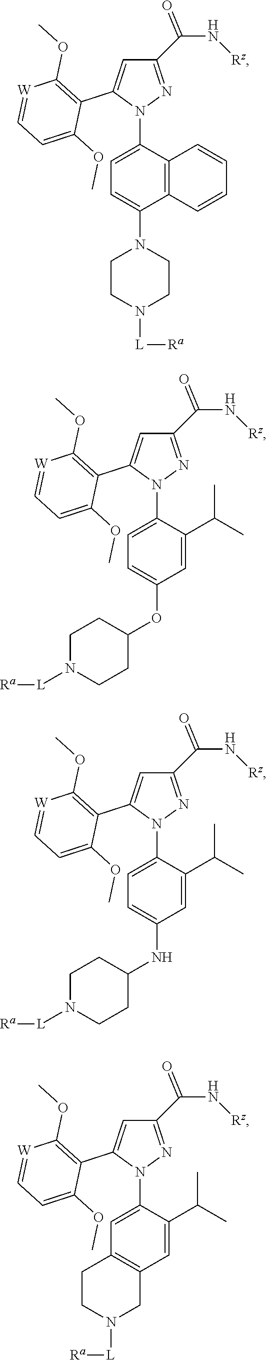

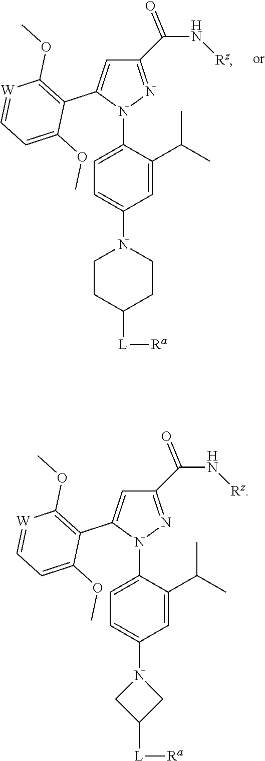

In some embodiments, the compound has the following structure:

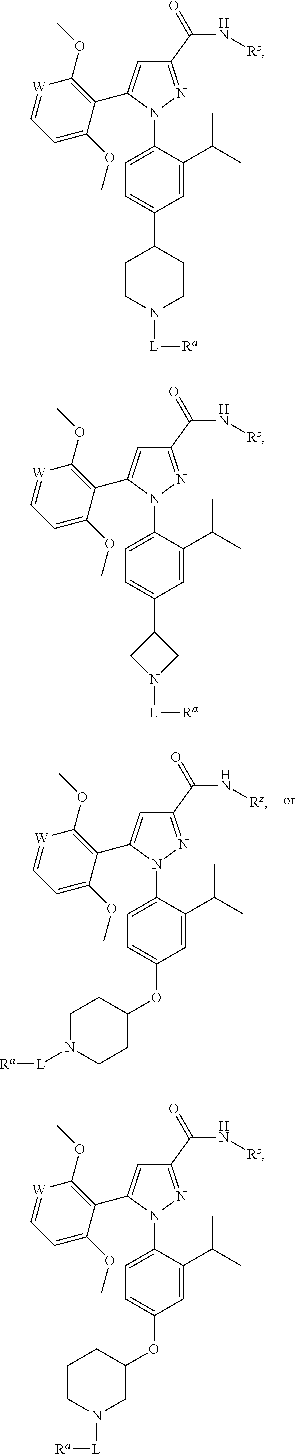

In some embodiments, W is —CH—. In some embodiments, W is N. In some embodiments, W is —C(F)—. In some embodiments, W is —C(OMe)-. In some embodiments, W is —C(OEt)-. In some embodiments, R^{z} is

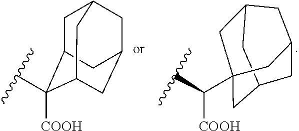 [SMILES: *C1(C(=O)O)C2CC3CC(C2)CC1C3 ; *[C@H](C(=O)O)C12CC3CC(CC(C3)C1)C2]

In some embodiments, the compound has the structure of Formula (IIa):

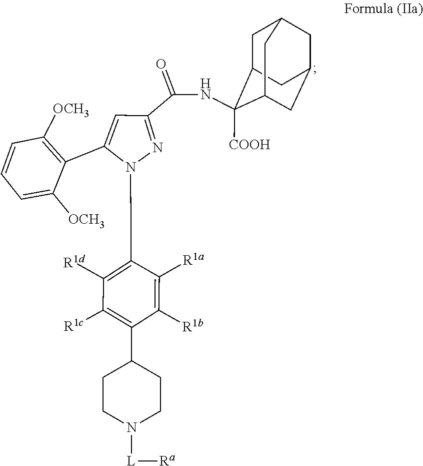

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, wherein the compound has the structure of Formula (IIb):

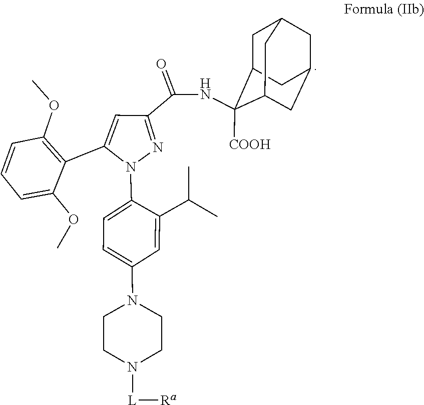

In some embodiments, the compound has the structure of Formula (IIc):

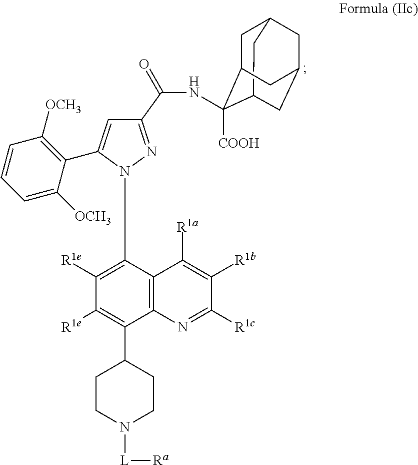

wherein R^{1a}, R^{1b}, R^{1c}, R^{1d}, R^{e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (IId):

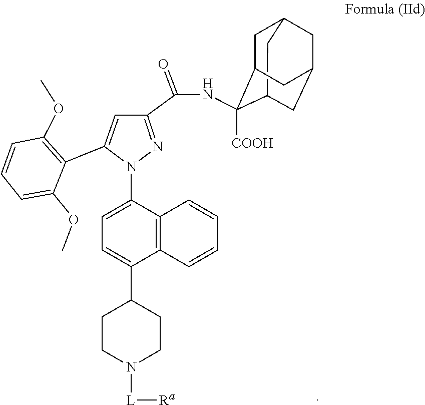

In some embodiments, the compound has the structure of Formula (IIe):

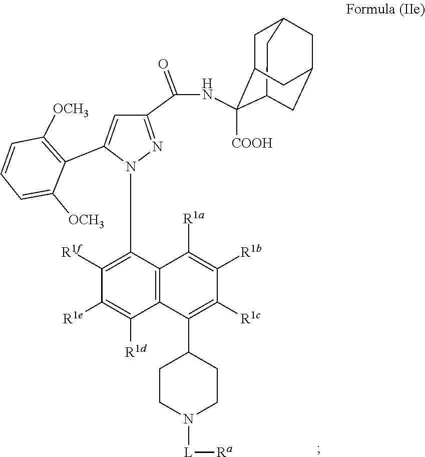

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (If):

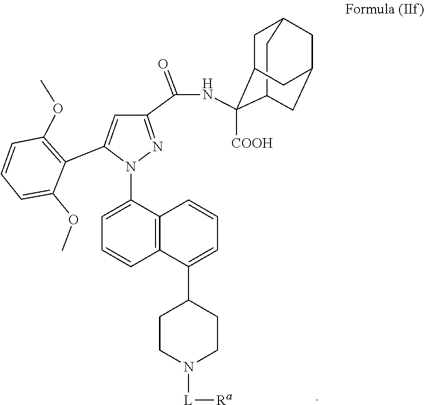

In some embodiments, the compound has the structure of Formula (IIg):

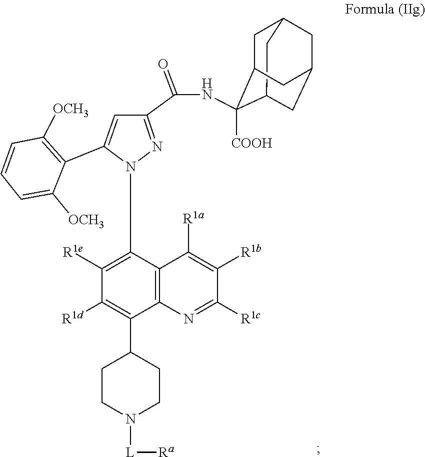

wherein R^{1a}, R^{1b}, R^{1c}, R^{1d}, and R^{1e} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (IIh):

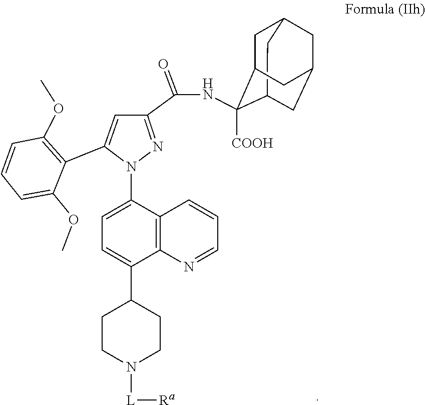

In some embodiments, the compound has the structure of Formula (IIi):

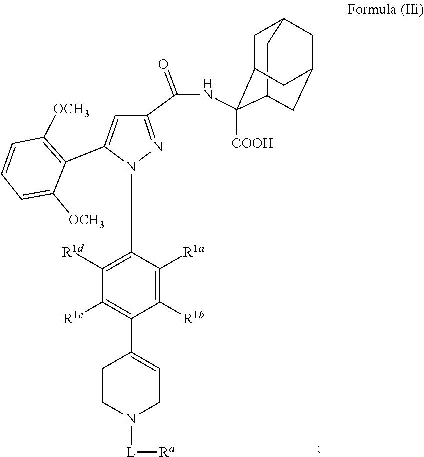

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, wherein the compound has the structure of Formula (IIj):

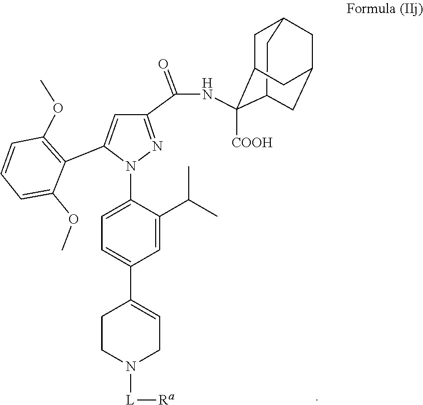

In some embodiments, the compound has the structure of Formula (IIk):

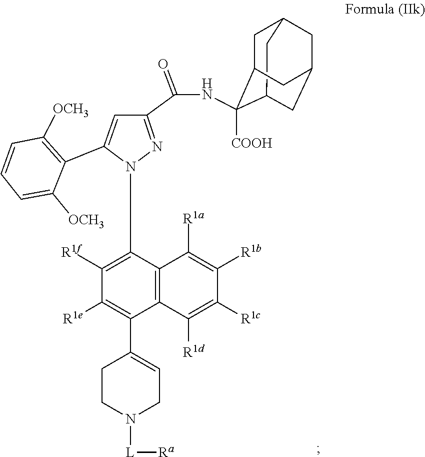

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IIl):

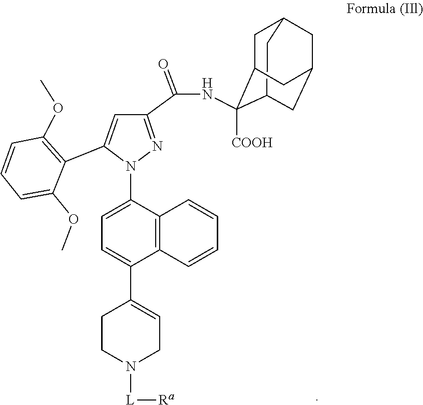

In some embodiments, the compound has the structure of Formula (IIm):

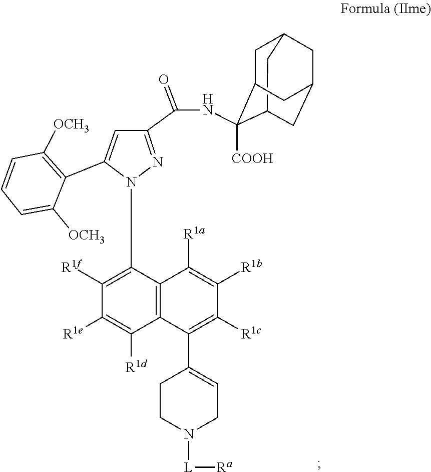

wherein R^{1a}, R^{1b}, R^{1c}, R^{1d}, R^{1c}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl

In some embodiments, the compound has the structure of Formula (IIn):

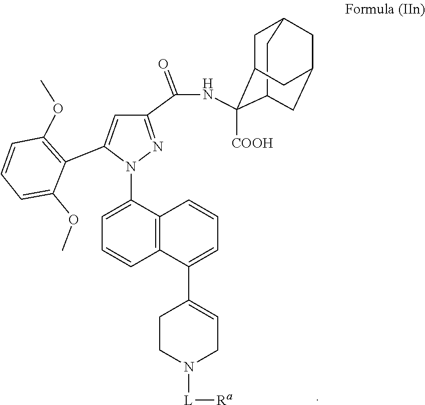

In some embodiments, the compound has the structure of Formula (IIo):

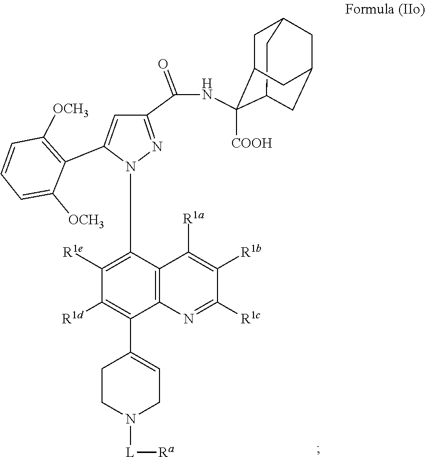

wherein R¹, R^{1b}, R^{1c}, R^{1d}, and R^{1e} are each independently selected from the group consisting of hydrogen, —F, —Cl, —Br, -L, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IIp):

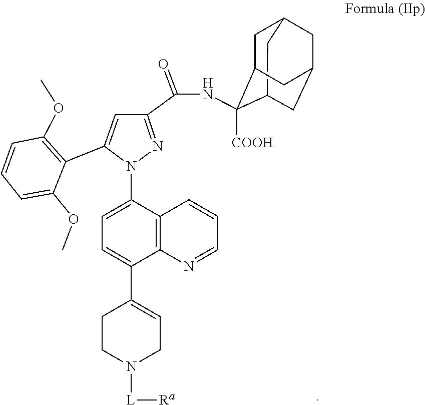

In some embodiments, the compound has the structure of Formula (IIIa):

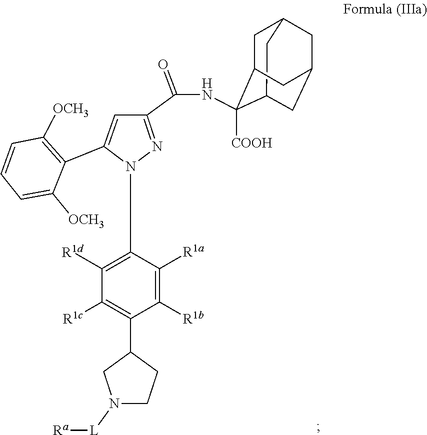

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IIIa-1):

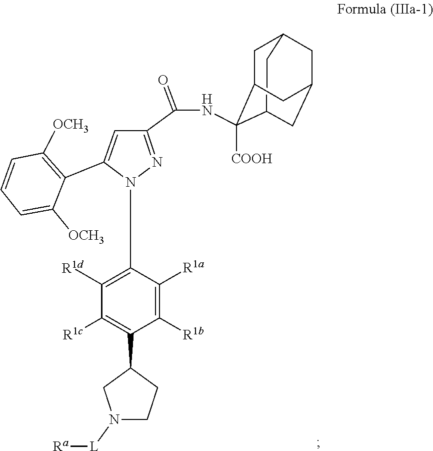

wherein R^{1a}, R^{1b}, RJC, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IIIa-2):

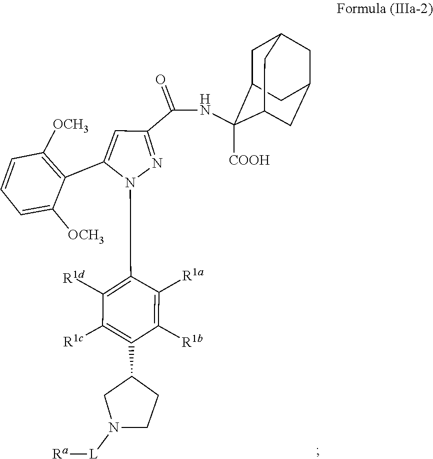

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IIIb):

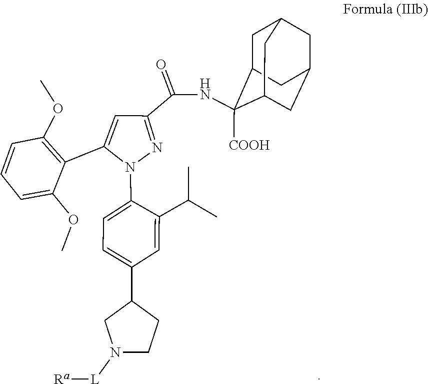

In some embodiments, the compound has the structure of Formula (IIIb-1):

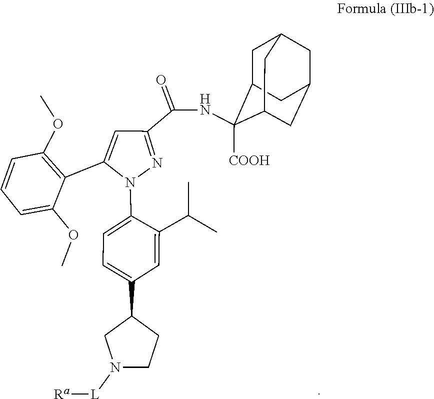

In some embodiments, the compound has the structure of Formula (IIIb-2):

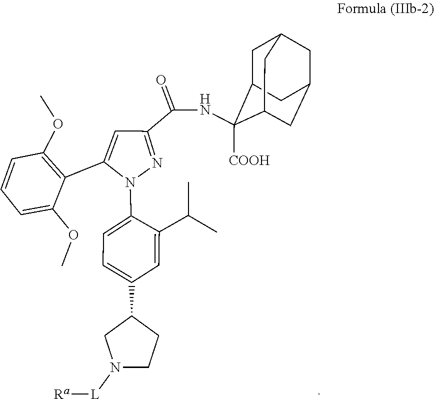

In some embodiments, the compound has the structure of Formula (IIIc):

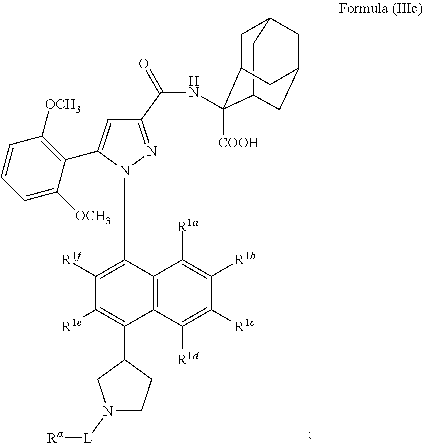

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IIIc-1):

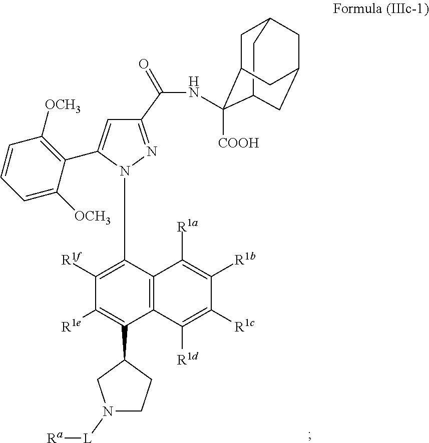

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (IIc-2):

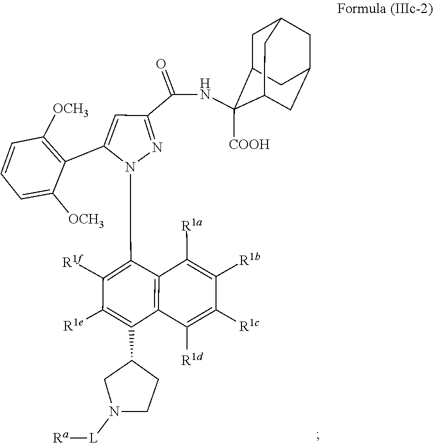

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IIId):

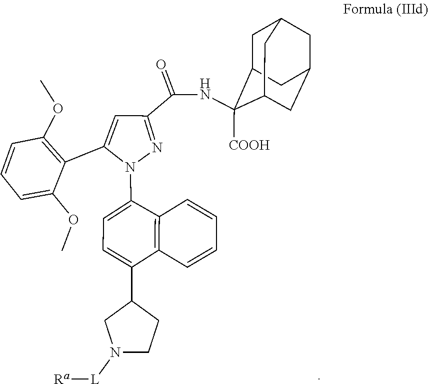

In some embodiments, the compound has the structure of Formula (IIId-1):

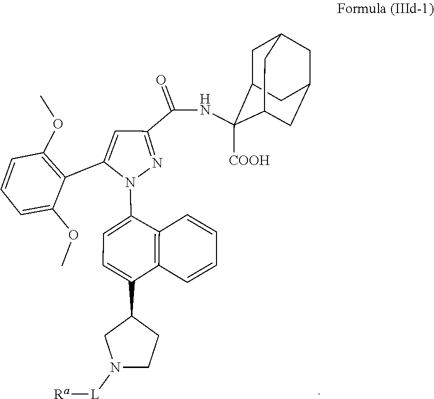

In some embodiments, the compound has the structure of Formula (IIId-2):

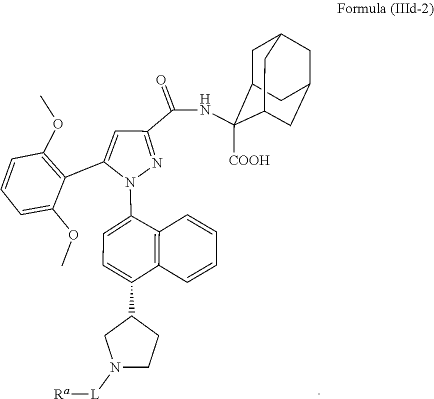

In some embodiments, the compound has the structure of Formula (IVa):

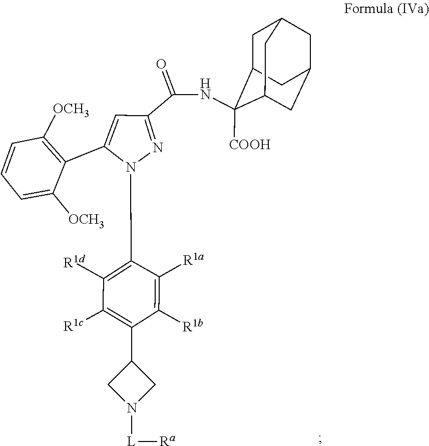

wherein R^{1a}, R^{1b}, R^{1c}, and R^{1d}, are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IVb):

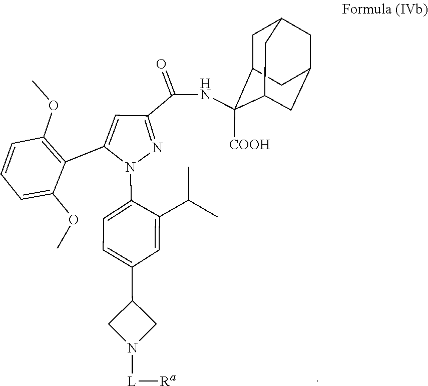

In some embodiments, the compound has the structure of Formula (IVc):

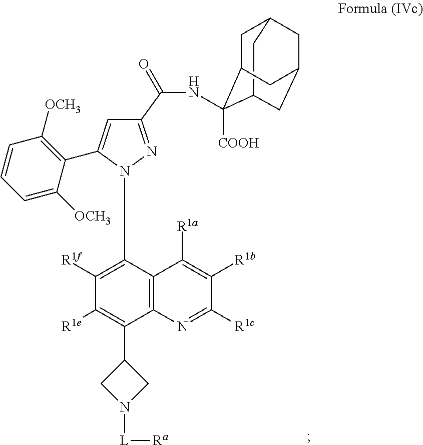

wherein R^{a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (IVd):

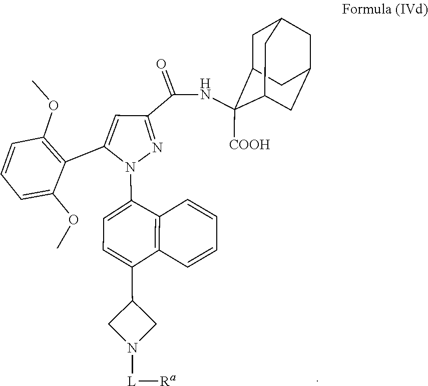

In some embodiments, the compound has the structure of Formula (Va):

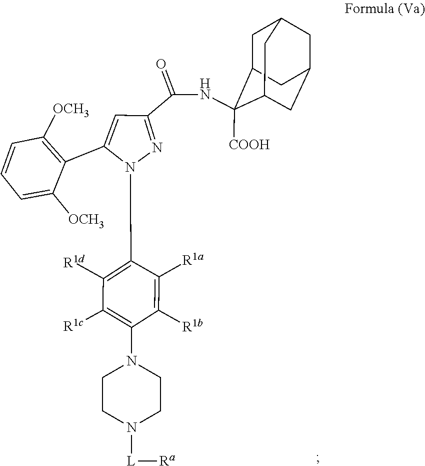

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (Vb):

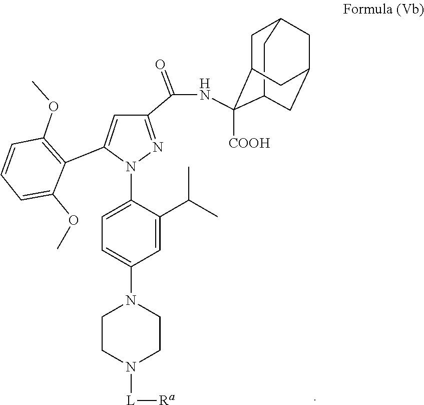

In some embodiments, the compound has the structure of Formula (Vc):

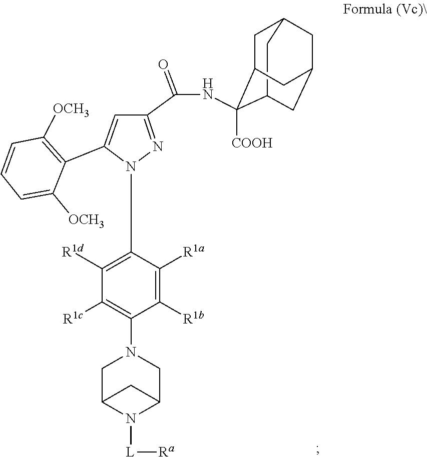

wherein R¹, R^{1b}, R^{1c}, and R^{1d} are each independently selected from the group consisting of hydrogen, —F, —Cl, —Br, -L, —OH, —CF₃, —CN, —CO₂H, —C(═O)OCH₃, —C(═O)NH₂, —CH(═O)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (Vd):

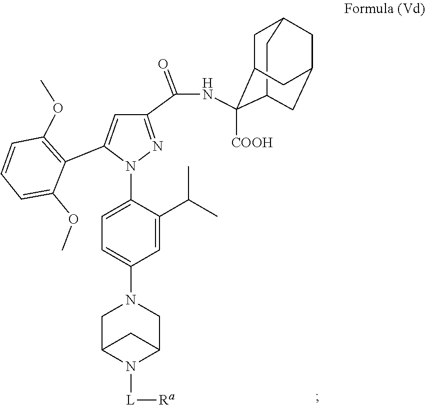

In some embodiments, the compound has the structure of Formula (Ve):

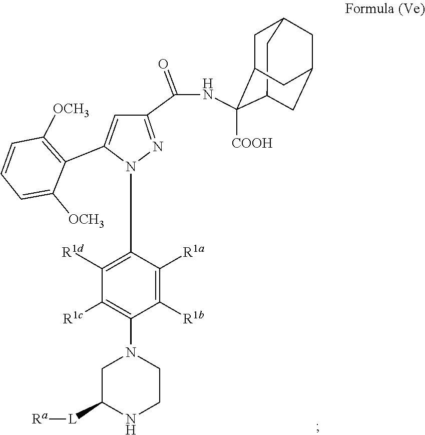

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (Vf):

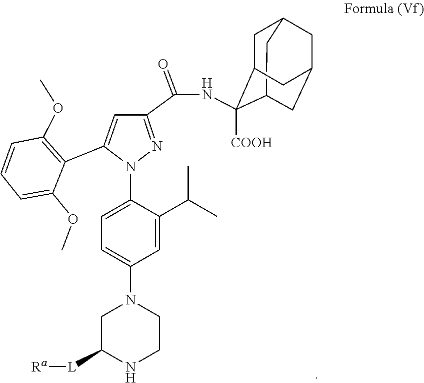

In some embodiments, the compound has the structure of Formula (Vg):

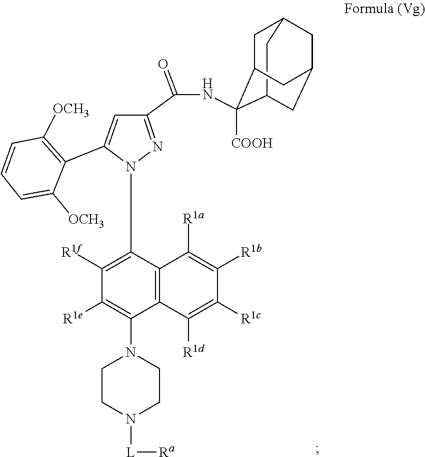

wherein R^{a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (Vh):

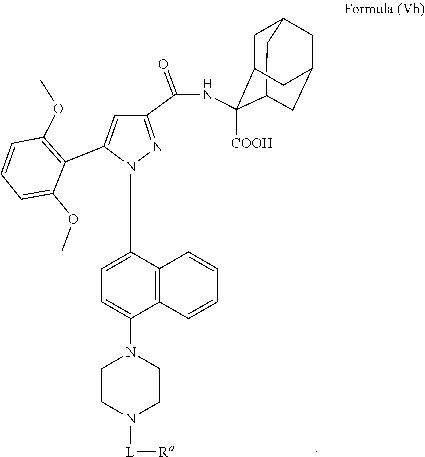

In some embodiments, the compound has the structure of Formula (Vi):

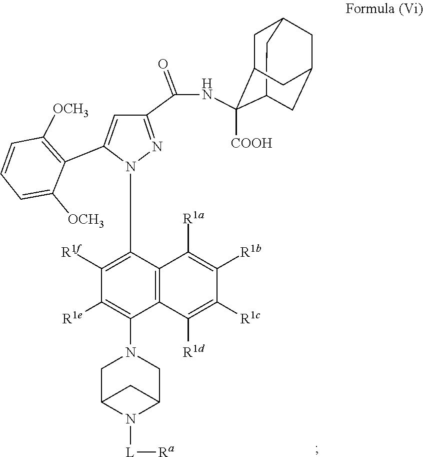

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (Vj):

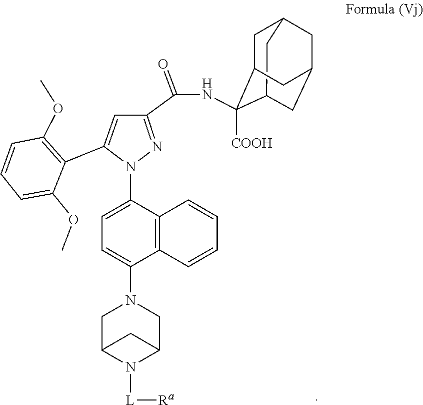

In some embodiments, the compound has the structure of Formula (Vk):

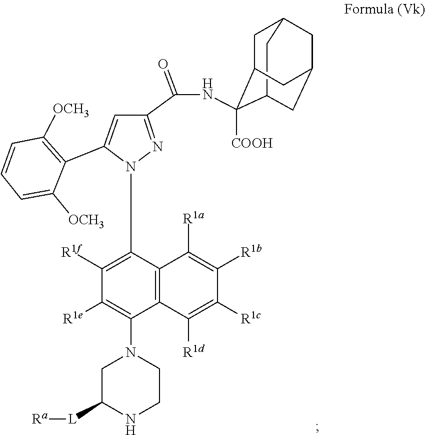

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂, —C(═O)OCH₃, —C(═O)N—H₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (Vl):

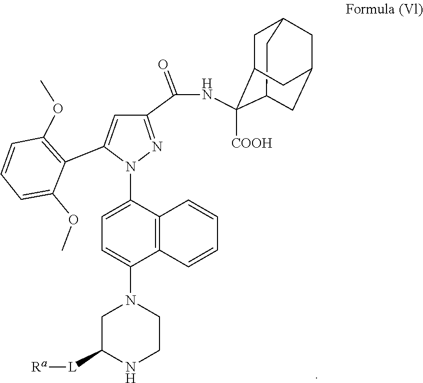

In some embodiments, the compound has the structure of Formula (VIa):

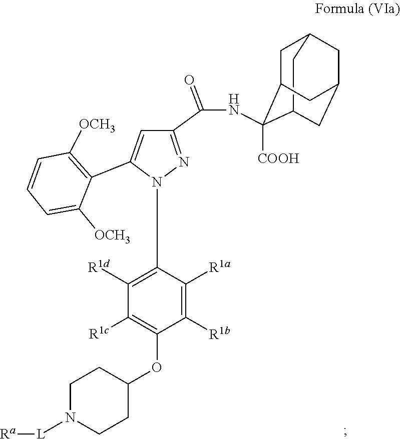

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (VIb):

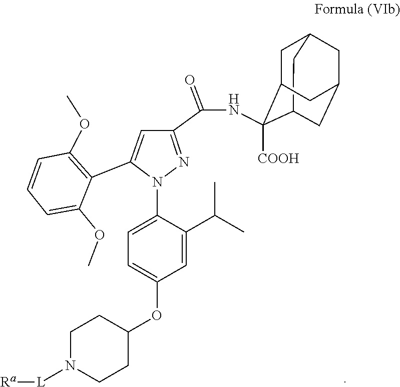

In some embodiments, the compound has the structure of Formula (VIc):

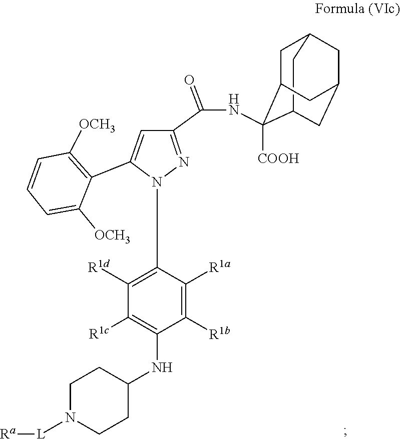

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (VId):

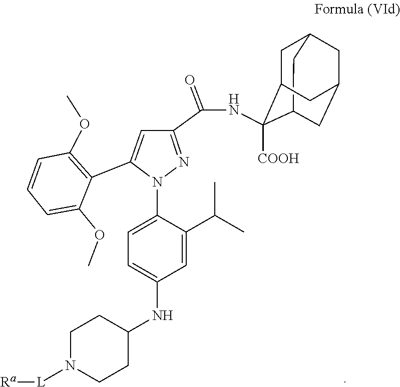

In some embodiments, the compound has the structure of Formula (VIe):

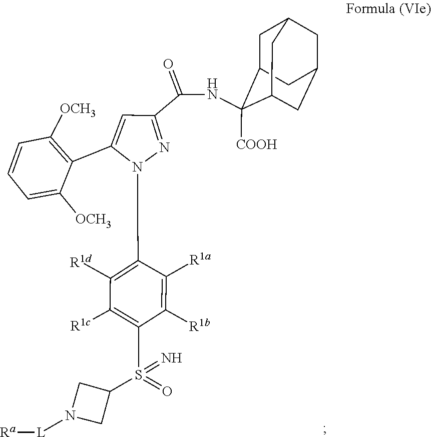

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═)CH₃, —NHCO₂H, —NHC(═O)OCH₃, —OC(═O)NH₂, and substituted or unsubstituted —C₁-C₆ alkyl.

In some embodiments, the compound has the structure of Formula (VIf):

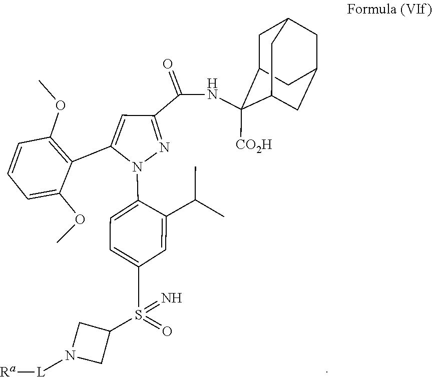

In some embodiments, the compound has the structure of Formula (VIg):

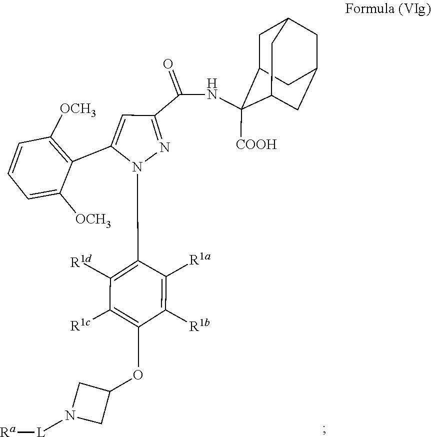

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (VIh):

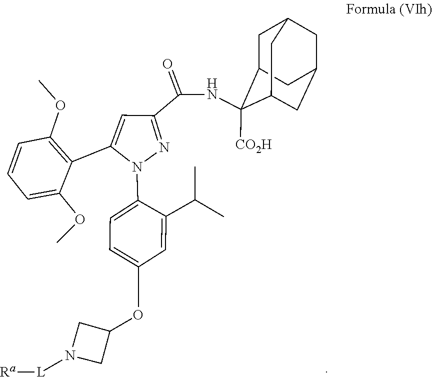

In some embodiments, the compound has the structure of Formula (VIIa):

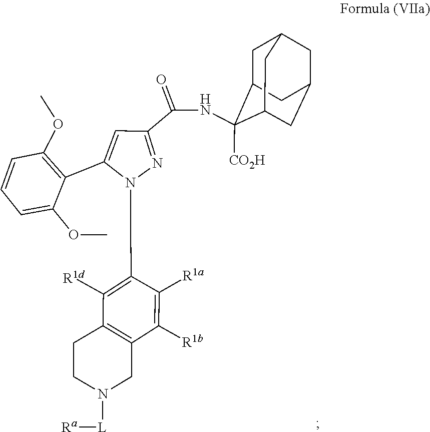

wherein R^{1a}, R^{1b}, and R^{1c} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (VIIb):

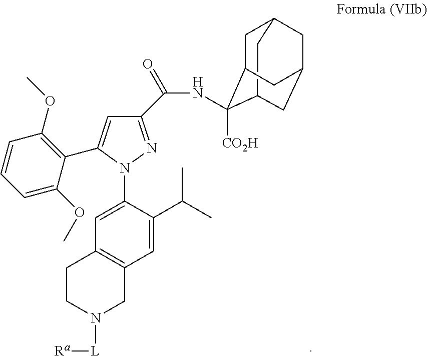

In some embodiments, the compound has the structure of Formula (VIIIa):

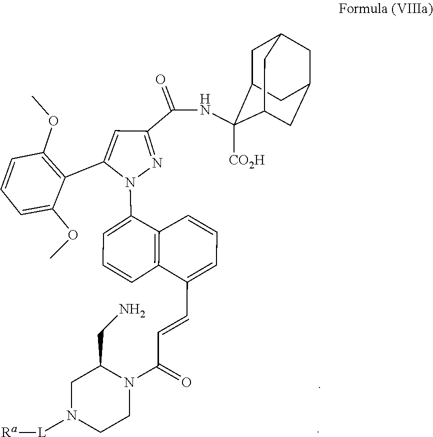

In some embodiments, the compound has the structure of Formula (VIIIb):

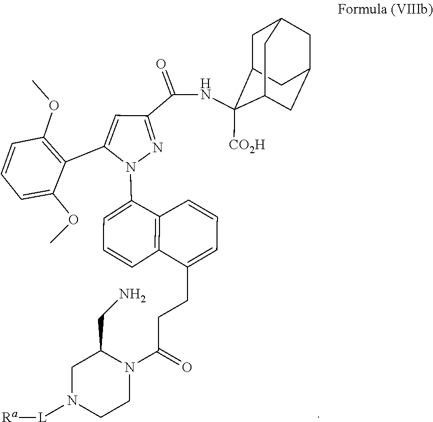

In some embodiments, the compound has the structure of Formula (IXa):

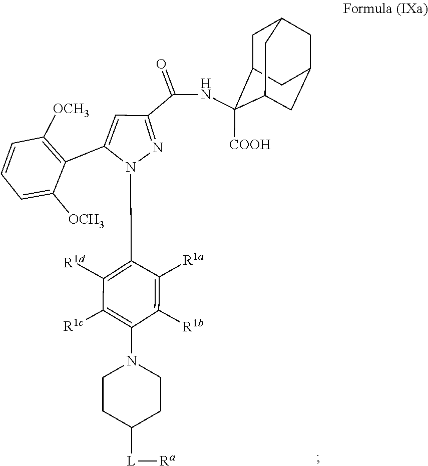

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (IXb):

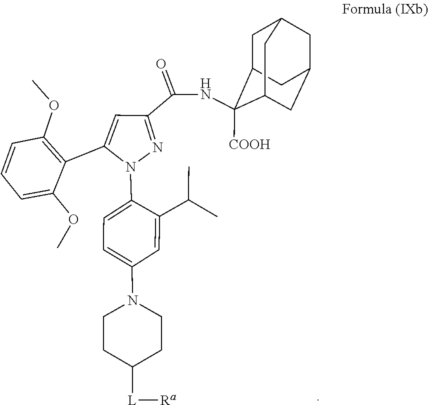

In some embodiments, the compound has the structure of Formula (Xa):

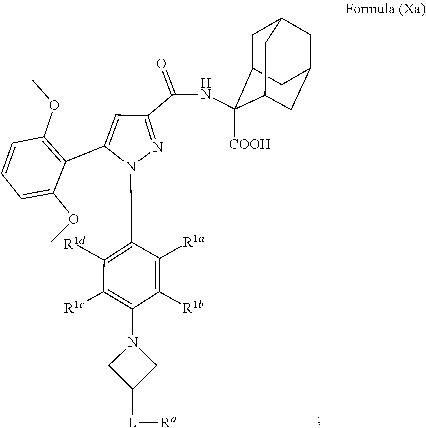

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (Xb):

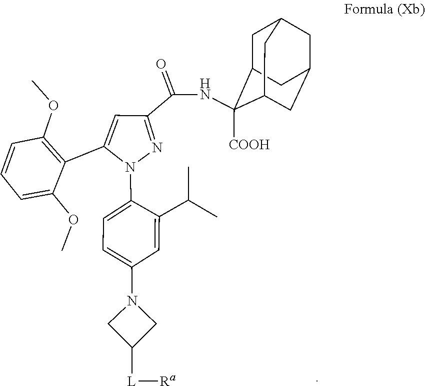

In some embodiments, the compound has the structure of Formula (XIa):

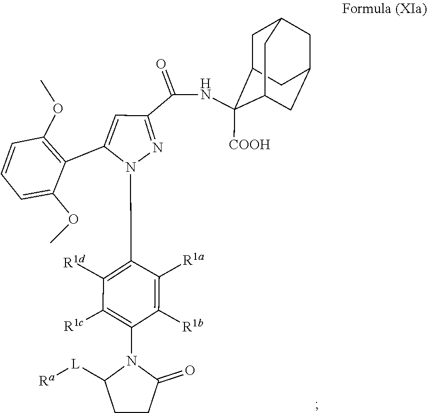

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═—N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, the compound has the structure of Formula (XIb):

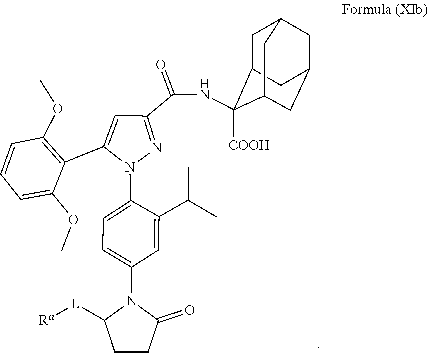

In some embodiments, the compound has the structure of Formula (XIIa):

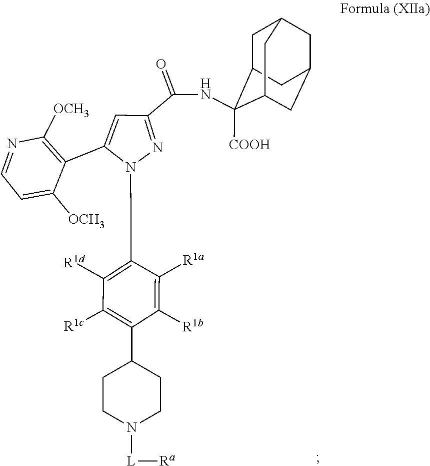

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, wherein the compound has the structure of Formula (XIIb):

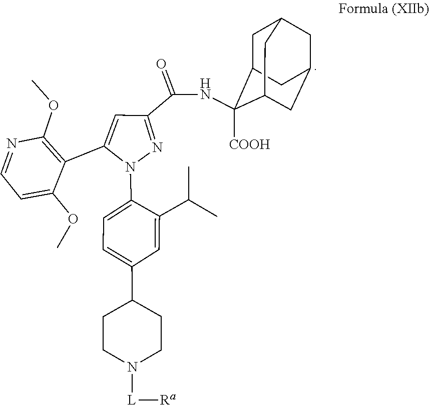

In some embodiments, the compound has the structure of Formula (XIIc):

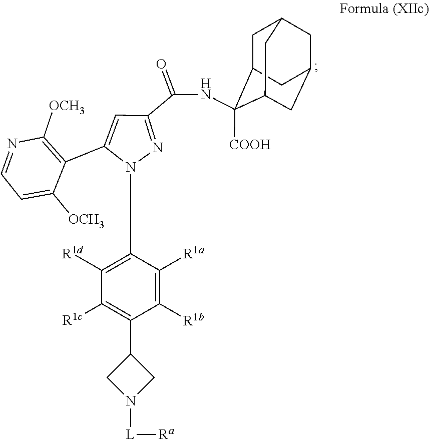

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, wherein the compound has the structure of Formula (XIId):

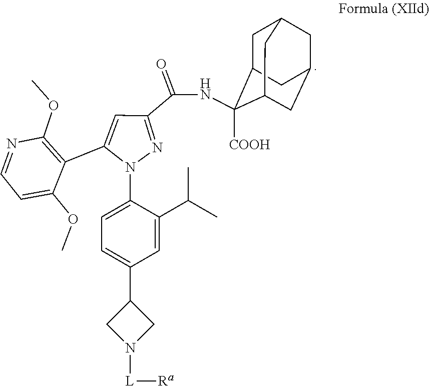

In some embodiments, the compound has the structure of Formula (XIIIa):

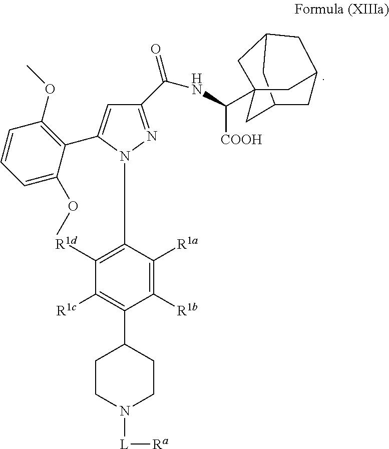

In some embodiments, the compound has the structure of Formula (XIIIb):

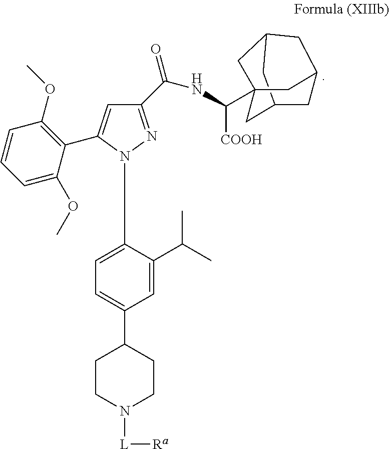

In some embodiments, the compound has the structure of Formula (XIVa):

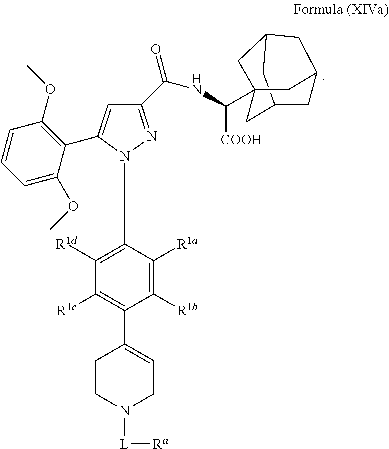

In some embodiments, the compound has the structure of Formula (XIVb):

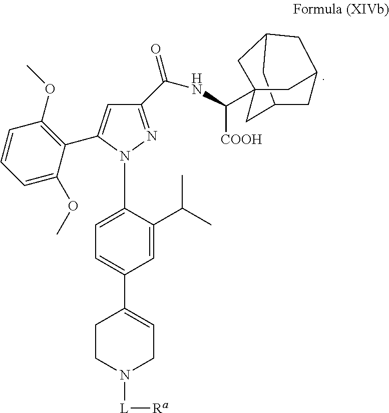

In some embodiments, the compound has the structure of Formula (XVa):

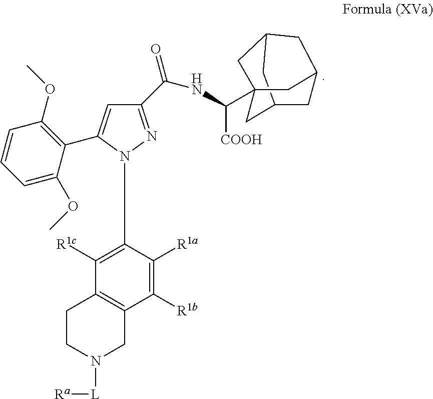

In some embodiments, the compound has the structure of Formula (XVb):

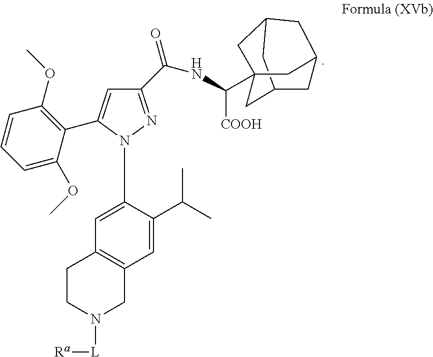

In some embodiments, the compound has the structure of Formula (XVIa):

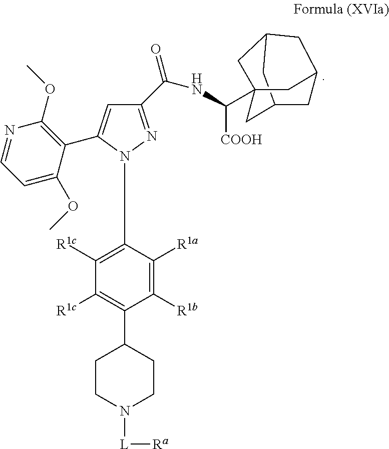

In some embodiments, the compound has the structure of Formula (XVIb):

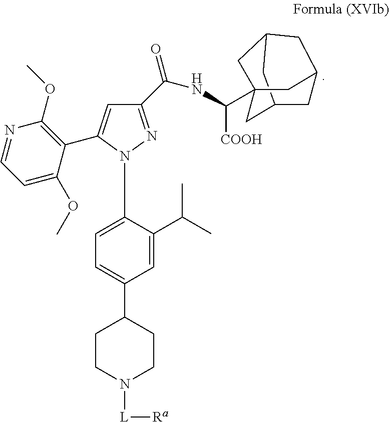

In some embodiments, the compound of Formula (A) or Formula (I) has the following structure, or a pharmaceutically acceptable salt thereof:

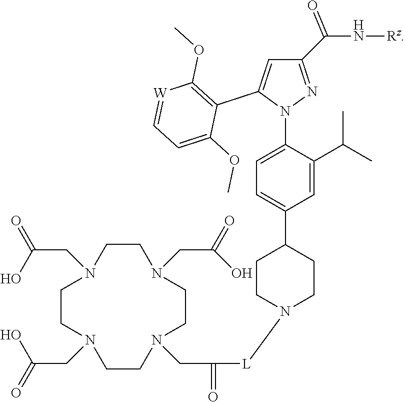

In some embodiments, the compound of Formula (A) or Formula (I) has the following structure, or a pharmaceutically acceptable salt thereof:

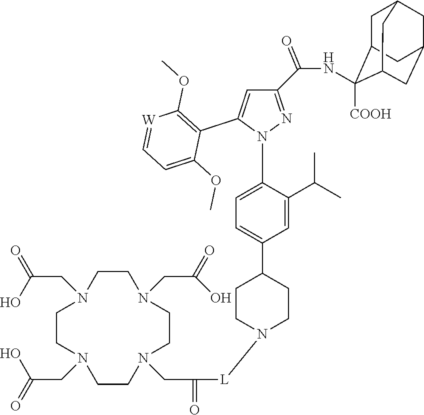

In some embodiments, W is —C(H)—, —C(F)—, —C(OMe)-, —C(OEt)-, or —N—. In some embodiments, W is —CH— or —N—, In some embodiments, L is: -L²-*, -L²-L⁴-*, -L²-L⁶-*, -L⁴-*, or -L²-L³-L⁶-*; wherein * denotes the attachment point of L to R¹.

In some embodiments, the compound of Formula (A) or Formula (I) has the following structure, or a pharmaceutically acceptable salt thereof:

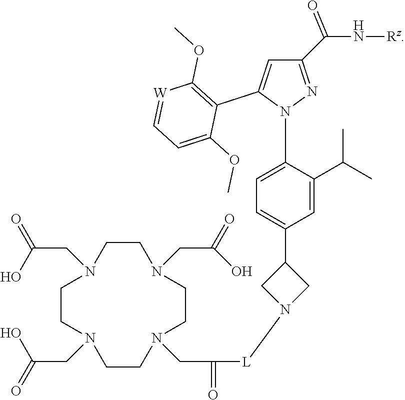

In some embodiments, the compound of Formula (A) or Formula (I) has the following structure, or a pharmaceutically acceptable salt thereof:

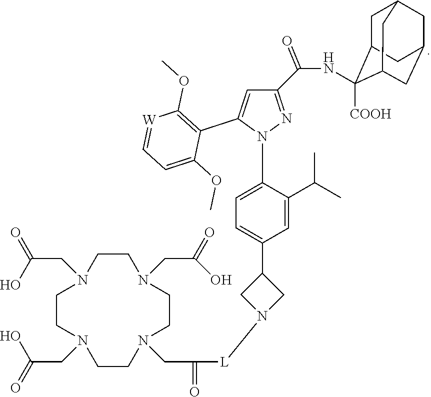

In some embodiments, W is —C(H)—, —C(F)—, —C(OMe)-, —C(OEt)-, or —N—. In some embodiments, W is —CH— or —N—. In some embodiments, L is: -L²-*, -L²-L⁴-*, -L²-L⁶-*, -L²-L³-L⁴-* or -L²-L³-L⁶-*; wherein * denotes the attachment point of L to R^{a}.

In some embodiments, the compound of Formula (A) or Formula (I) has the following structure, or a pharmaceutically acceptable salt thereof:

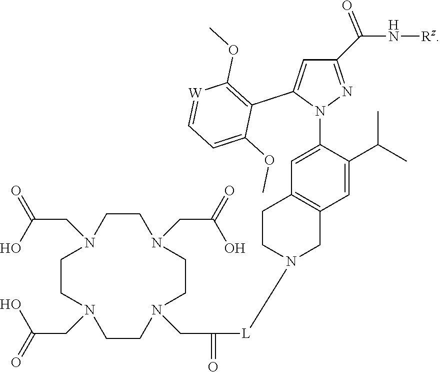

In some embodiments, the compound of Formula (A) or Formula (I) has the following structure, or a pharmaceutically acceptable salt thereof.

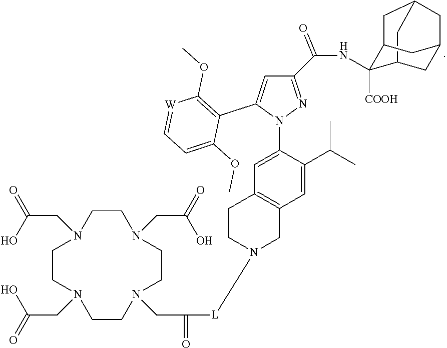

In some embodiments, W is —C(H)—, —C(F)—, —C(OMe)-, —C(OEt)-, or —N—, In some embodiments, W is —CH— or —N—. In some embodiments, L is: -L²-*, -L²-L⁴-*, -L²-L⁶-*, -L²-L³-L⁴-*, or -L²-L³-L⁶-*; wherein * denotes the attachment point of L to R^{a}.

In some embodiments, the compound of Formula (A) or Formula (I) has the following structure, or a pharmaceutically acceptable salt thereof:

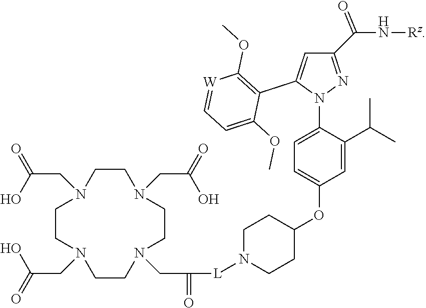

In some embodiments, the compound of Formula (A) or Formula (I) has the following structure, or a pharmaceutically acceptable salt thereof:

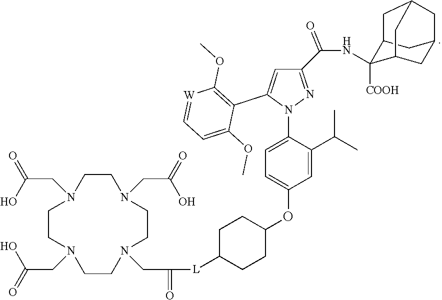

In some embodiments, W is —C(H)—, —C(F)—, —C(OMe)-, —C(OEt)-, or —N—. In some embodiments, W is —CH— or —N—. In some embodiments, L is: -L²-*, -L²-L⁴-*, -L²-L⁶-*, -L²-L³-L⁴-*, or -L²-L³-L⁶-*; wherein * denotes the attachment point of L to R^{a}.

### Radionuclide Complexes

Radiopharmaceuticals have increasingly become very useful tools for physicians to diagnose, stage, treat, and monitor the progression of several diseases, especially cancer. The primary difference between radiopharmaceuticals and other pharmaceutical drugs is that radiopharmaceuticals contain a radionuclide. The nuclear decay properties of the radionuclide determine whether a radiopharmaceutical will be used clinically as a diagnostic agent or as a therapeutic agent. Diagnostic radiopharmaceuticals require radionuclides that emit either gamma (γ) rays or positrons (β+), which subsequently annihilate with nearby electrons to produce two 511 keV annihilation photons emitted approximately 180° away from each other. Gamma ray-emitting radionuclides (e. g. ^{99m}Tc, ¹¹¹In, ²⁰¹Tl, etc.) are useful for single photon emission computed tomography (SPECT), while positron-emitting radionuclides (e. g. ¹⁸F, ⁸⁹Zr, ⁶⁸Ga, etc.) are useful for positron emission tomography (PET).

In contrast, therapeutic radiopharmaceuticals require radionuclides that emit particulate radiation, such as alpha (α) particles, beta (β−) particles, or Auger electrons. These particles, which strongly interact with target tissues (e. g. cancerous tumor) and lead to extensive localized ionization, can damage chemical bonds in DNA molecules and potentially induce cytotoxicity.

For most nuclear medicine applications, it is desired that a diagnostic radiopharmaceutical is paired with a therapeutic radiopharmaceutical. This concept is commonly known as “theranostics”. As a first step in the theranostic concept, a target molecule labeled with a diagnostic radionuclide is used for quantitative imaging of a tumor imaging biomarker, either by positron emission tomography (PET) or single photon emission computed tomography (SPECT). When it is demonstrated that, with this targeted molecule, a tumoricidal radiation absorbed dose can be delivered to tumor and metastases, as a second step, via administration of the same or a similar target molecule labeled with a therapeutic radionuclide.

In some embodiments, the chemical and pharmacokinetic behaviors of both the diagnostic and therapeutic radiopharmaceuticals match. In some embodiments, the diagnostic and therapeutic radionuclides are a chemically identical radioisotope pair (also known as a “matched pair”). One examples of a matched pair for theranostic radiopharmaceutical applications is the ¹²³I/¹³¹I pair, where ¹²³I-labeled compounds are used for diagnosis, while ¹³¹I-labeled compounds are used for therapy. Other theranostic matched pairs include ⁴⁴Sc/⁴⁷Sc, ⁶⁴Cu/⁶⁷Cu, ⁷²As/⁷⁷As, ⁸⁶Y/⁹⁰Y, and ²⁰³Pb/²¹²Pb, among others. Alternatively, radionuclide pairs from different elements can be utilized for theranostic radiopharmaceutical development when their chemistry is very similar (e. g. ^{99m}Tc/^{186/188}Re) and there is no significant difference in the pharmacokinetic behavior between the diagnostic and therapeutic analogues. Another example is the ⁶⁸Ga/¹⁷⁷Lu pair, where ⁶⁸Ga is used for diagnosis and ¹⁷⁷Lu is used for therapy. For example, gastroenteropancreatic endocrine tumors express high amounts of sst2 receptor that can be targeted with somatostatin receptor scintigraphy for diagnostic purposes with a ⁶⁸Ga sst2 ligand conjugate ([⁶⁸Ga]Ga-DOTA-TATE (NETSPOT™) or [⁶⁸Ga]Ga-DOTA-TOC (DOTA-(D-Phe1,Tyr3)-octreotide, SomaKit TOC®)), followed by treatment with a ¹⁷⁷Lu sst2 ligand conjugate ([177Lu]Lu-DOTA-TATE) for endoradiotherapy.

Chelating Moieties used to Generate Metal (Radionuclide) Complexes

The compounds described herein comprise at least one R^{a} group, wherein R^{a} is a chelating moiety capable of chelating a radionuclide (Z′), or radionuclide complex thereof. In some embodiments, any suitable group or atom(s) of the chelator are used to connect, via an optional linker, to the NTSR1 targeting ligand.

In some embodiments, the chelator is capable of binding a radioactive atom. In some embodiments, the binding is direct, e.g., the chelator makes hydrogen bonds or electrostatic interactions with a radioactive atom. In some embodiments, the binding is indirect, e.g., the chelator binds to a molecule that comprises a radioactive atom. In some embodiments, the chelator is or comprises a macrocycle.

In some embodiments, the chelator comprises one or more amine groups. In some embodiments, the metal chelator comprises two or more amine groups. In some embodiments, the chelator comprises three or more amine groups. In some embodiments, the chelator comprises four or more amine groups. In some embodiments, the chelator includes 4 or more N atoms, 4 or more carboxylic acid groups, or a combination thereof. In some embodiments, the chelator does not comprise S. In some embodiments, the chelator comprises a ring. In some embodiments, the ring comprises an O and/or a N atom. In some embodiments, the chelator is a ring that includes 3 or more N atoms, 3 or more carboxylic acid groups, or a combination thereof. In some embodiments, the chelator is polydentate ligand, bidentate ligand, or monodentate ligand. Polydentate ligands range in the number of atoms used to bond to a metal atom or ion. EDTA, a hexadentate ligand, is an example of a polydentate ligand that has six donor atoms with electron pairs that can be used to bond to a central metal atom or ion. Bidentate ligands have two donor atoms which allow them to bind to a central metal atom or ion at two points. Ethylenediamine (en) and the oxalate ion (ox) are examples of bidentate ligands.

In some embodiments, a chelator described herein comprises a cyclic chelating agent or an acyclic chelating agent. In some embodiments, a chelator described herein comprises a cyclic chelating agent. In some embodiments, a chelator described herein comprises an acyclic chelating agent.

In some embodiments, a chelator described herein comprises cyclen, DO2A, DO3A, HP-DO3A, DO3A-Nprop, DO3AP, DO3AP^{PrA}, DO3AP^{ABn}, DO3AM^{nBu}, BT-DO3A, DOTA, PSC, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis-DO2A2P, trans-DO2A2P, DOTEP, p-NO₂—Bn-DOTA, BAT, DO3TMP-Monoamide, CHX-A″-DTPA, c-DEPA, PCTA, p-NO₂—Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, HE₆phospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG₂DAP.

In some embodiments, a chelator described herein comprises DOTA, DOTAGA, DOTA(GA)₂, NOTA, NODAGA, TRITA, TETA, DOTA-MA, HP-DO3A, DOTMA, DOTA-pNB, DOTP, DOTMIP, DOTEP, DOTMPE, F-DOTPME, DOTPP, DOTBzP, DOTA-monoamide, BAT, DO3TMP-Monoamide, and CHX-A″-DTPA.

In some embodiments, a chelator described herein comprises DTA, CyEDTA, EDTMP, DTPMP, DTPA, CyDTPA, Cy2DTPA, DTPA-MA, DTPA-BA, and BOPA.

In some embodiments, a chelator described herein comprises DOTA, DOTAGA, DOTA(GA)₂, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂—Bn-PCTA, p-NO₂—Bn-DOTA, symPC2APA, symPCA2PA, asymPC2APA, asymPCA2PA, TRAP, AAZTA, DATA^{m}, THP, HEHA, HBED, or HBED-CC TFP.

In some embodiments, a chelator described herein comprises DOTA, NOTA, NODAGA, DOTAGA, HBED, HBED-CC TFP, H2DEPDPA, DFO-B, Deferiprone, CP256, YM103, TETA, CB-TE2A, TE2A, Sar, DiAmSar, TRAPH, TRAP-Pr, TRAP-OH, TRAP-Ph, NOPO, DEADPA, PCTA, EDTA, PEPA, HEHA, DTPA, EDTMP, AAZTA, DO3AP, DO3AP^{PrA}, DO3AP^{ABn}, or DOTAM.

In some embodiments, the chelator is or comprises DOTA, HBED-CC, DOTAGA, DOTA(GA)₂, NOTA, and DOTAM. In some embodiments, the chelator is or comprises NODAGA, NOTA, DOTAGA, DOTA(GA)₂, TRAP, NOPO, NCTA, DFO, DTPA, and HYNIC.

In some embodiments, the chelator comprises a macrocycle, e.g., a macrocycle comprising an O and/or a N atom, DOTA, HBED-CC, DOTAGA, DOTA(GA)₂, NOTA, DOTAM, one or more amines, one or more ethers, one or more carboxylic acids, EDTA, DTPA, TETA, DO3A, PCTA, or desferrioxamine.

In some embodiments, a metal chelator described herein comprises one of the following structures:

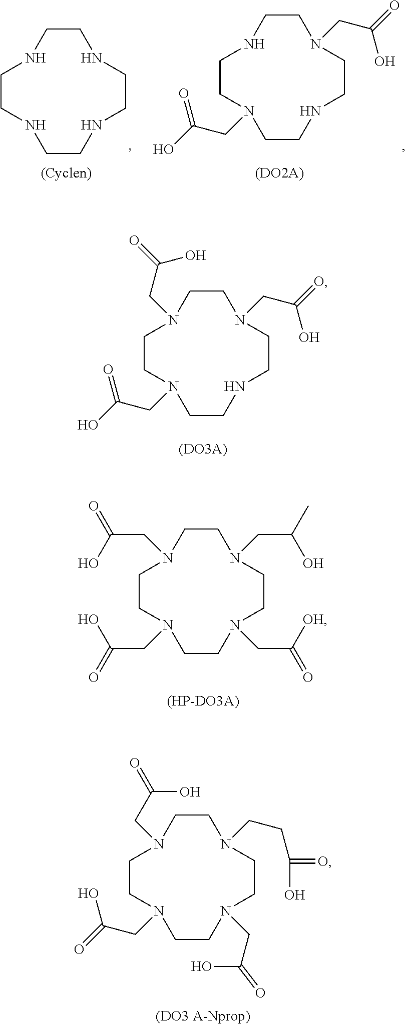

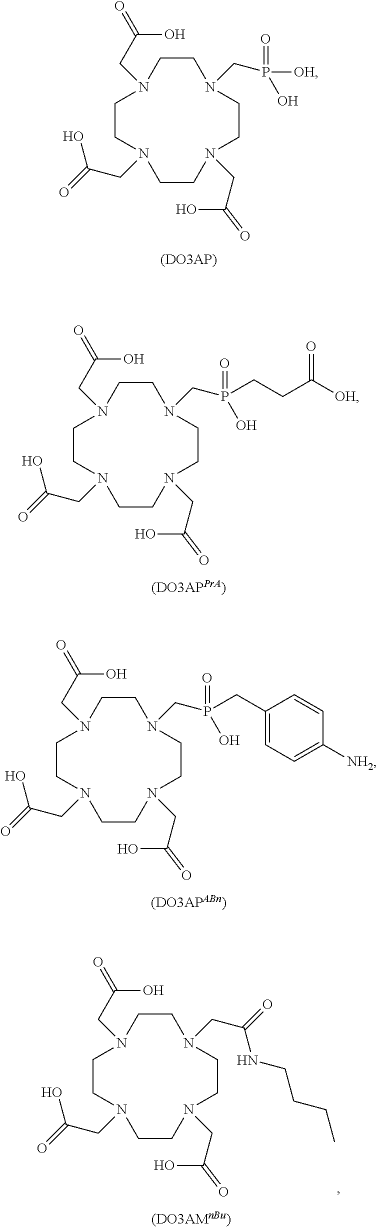 [SMILES: O=C(O)CN1CCN(CC(=O)O)CCN(CP(=O)(O)O)CCN(CC(=O)O)CC1 ; O=C(O)CCP(=O)(O)CN1CCN(CC(=O)O)CCN(CC(=O)O)CCN(CC(=O)O)CC1 ; Nc1ccc(CP(=O)(O)CN2CCN(CC(=O)O)CCN(CC(=O)O)CCN(CC(=O)O)CC2)cc1 ; CCCCNC(=O)CN1CCN(CC(=O)O)CCN(CC(=O)O)CCN(CC(=O)O)CC1]

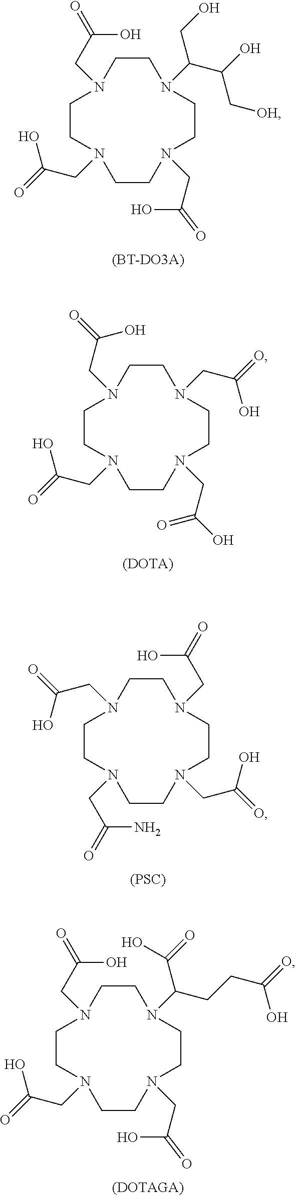

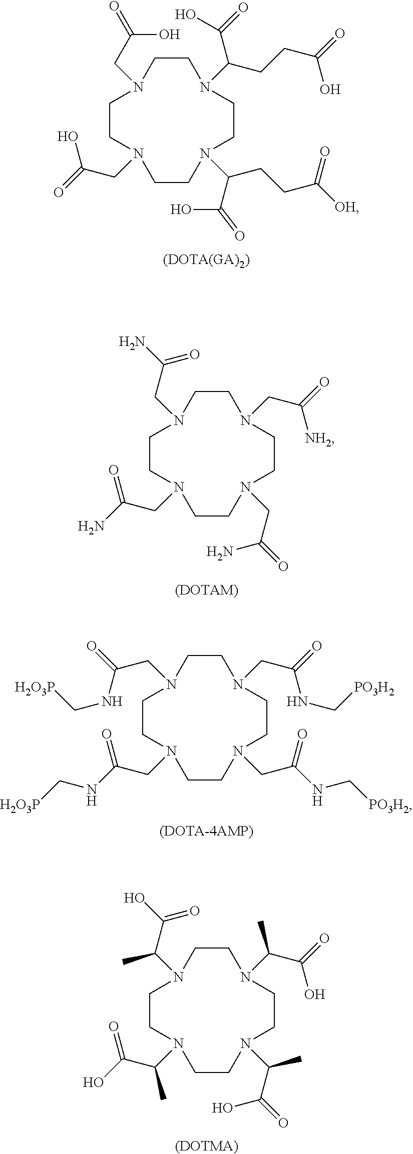

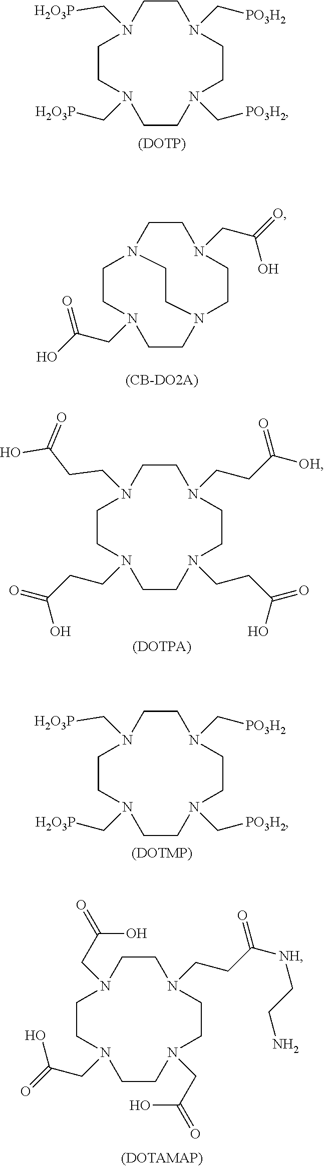

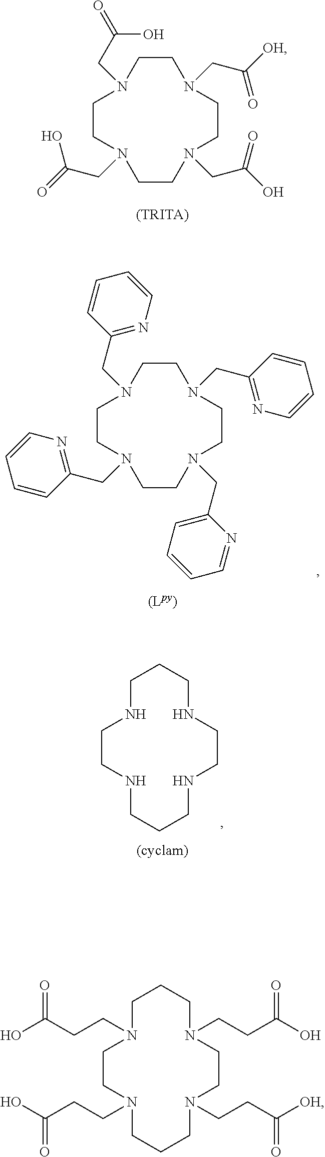

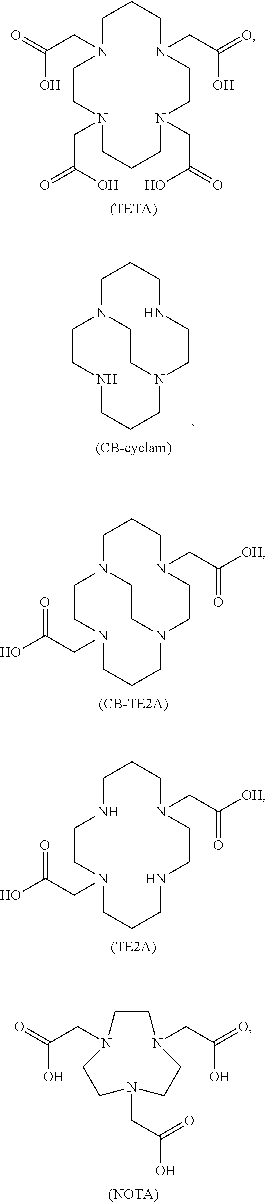

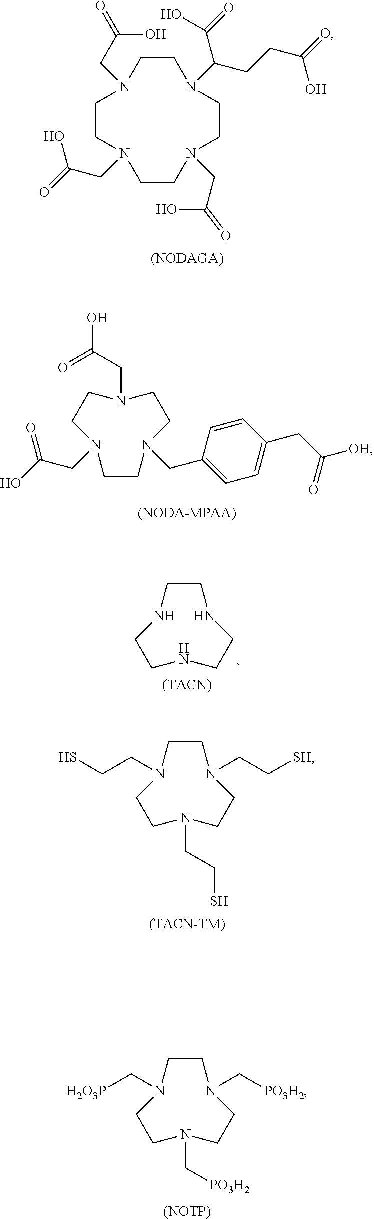

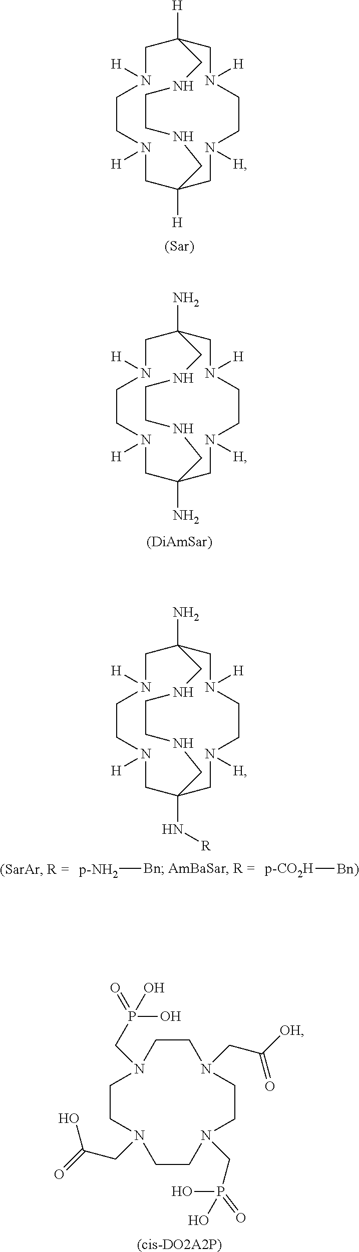

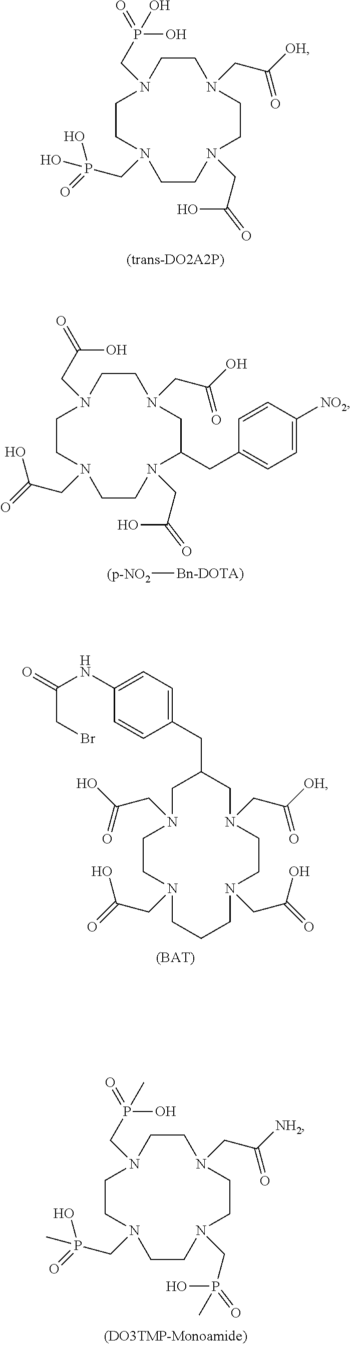

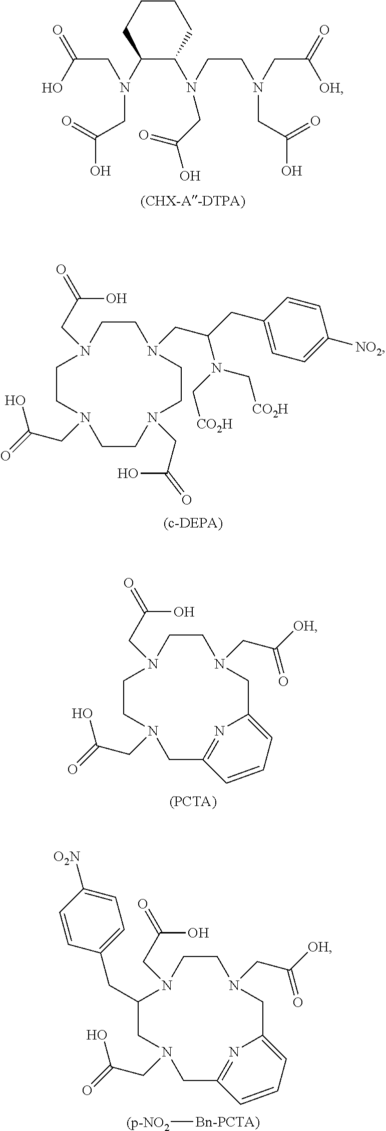

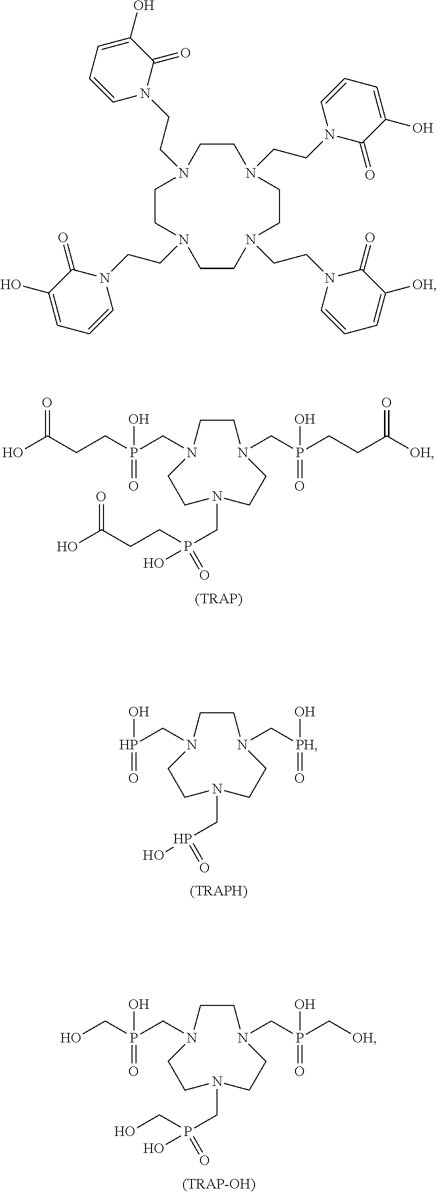

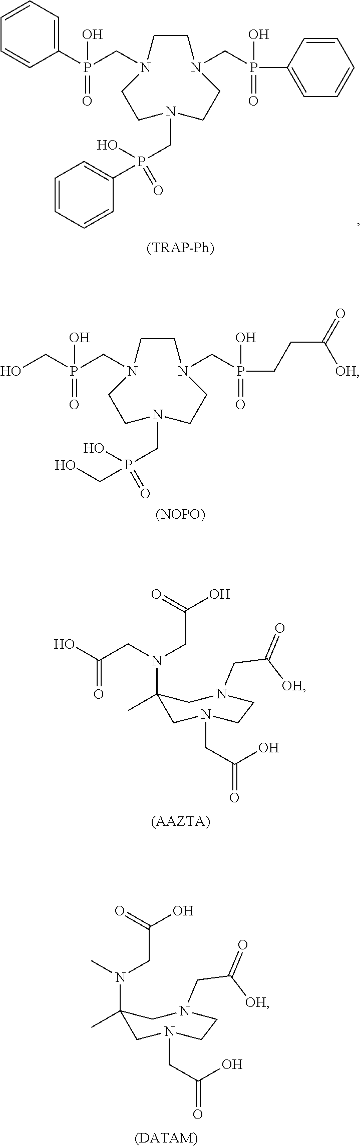

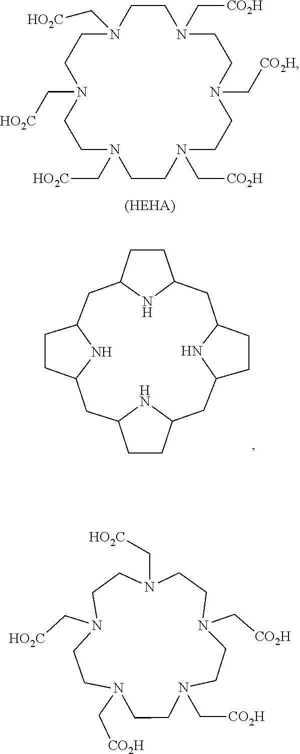 [SMILES: O=C(O)CN1CCN(CC(=O)O)CCN(CC(=O)O)CCN(CC(=O)O)CCN(CC(=O)O)CCN(CC(=O)O)CC1 ; C1CC2CC3CCC(CC4CCC(CC5CCC(CC1N2)N5)N4)N3 ; O=C(O)CN1CCN(CC(=O)O)CCN(CC(=O)O)CCN(CC(=O)O)CCN(CC(=O)O)CC1]

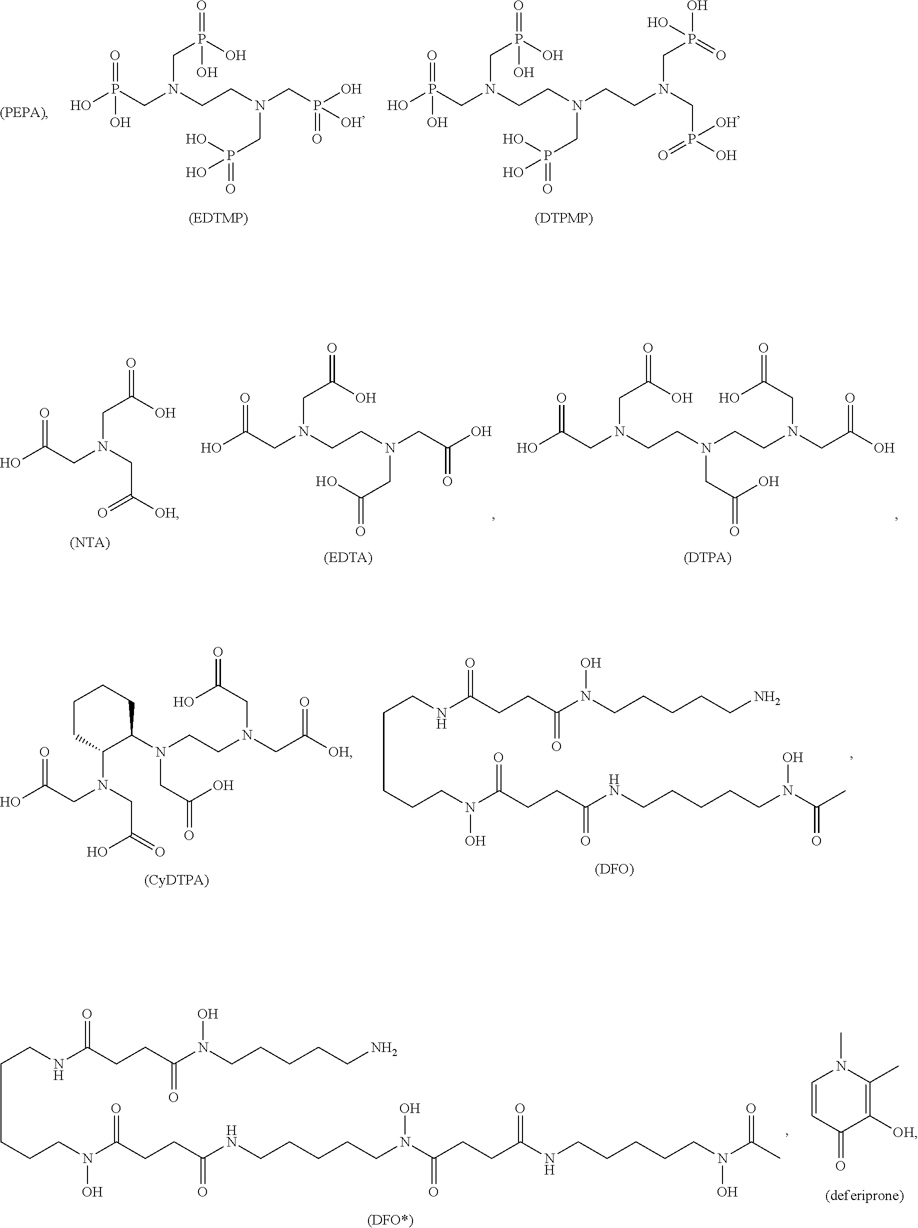

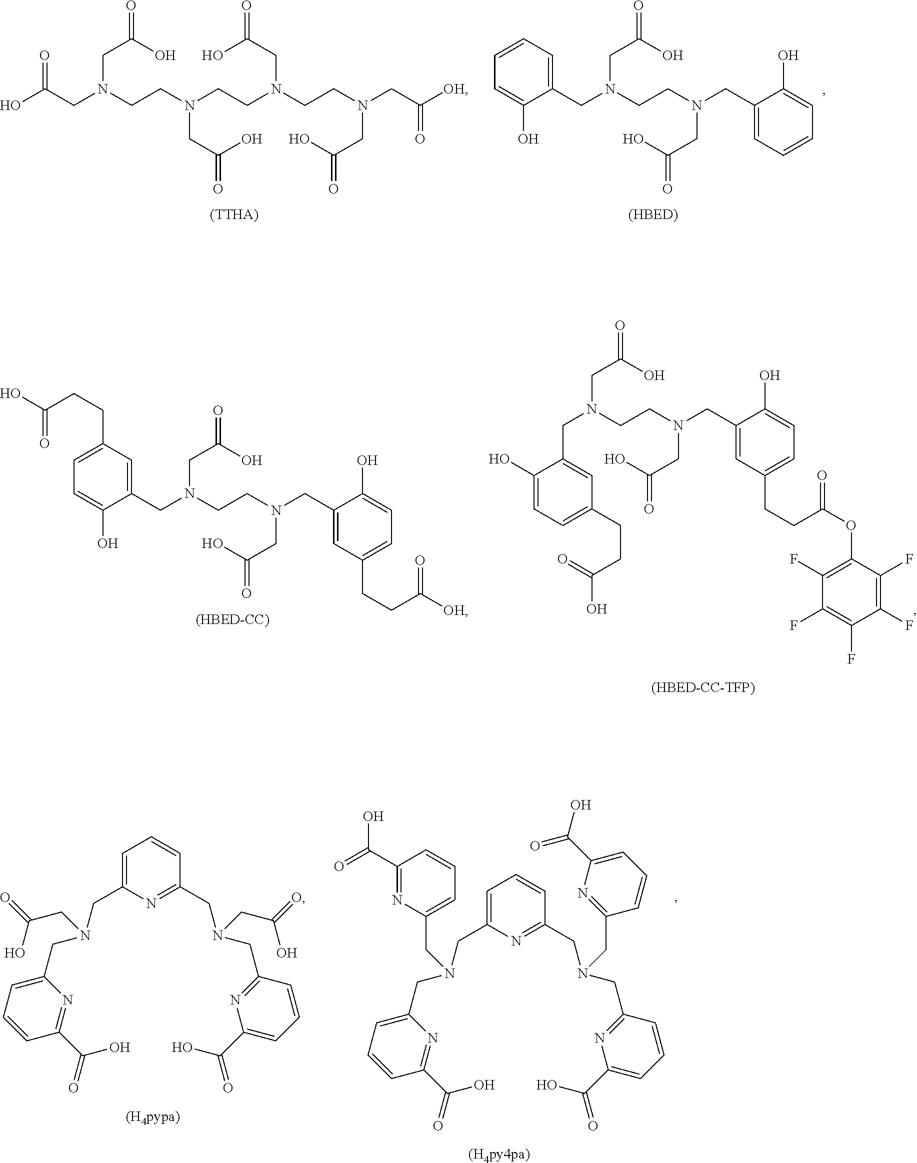

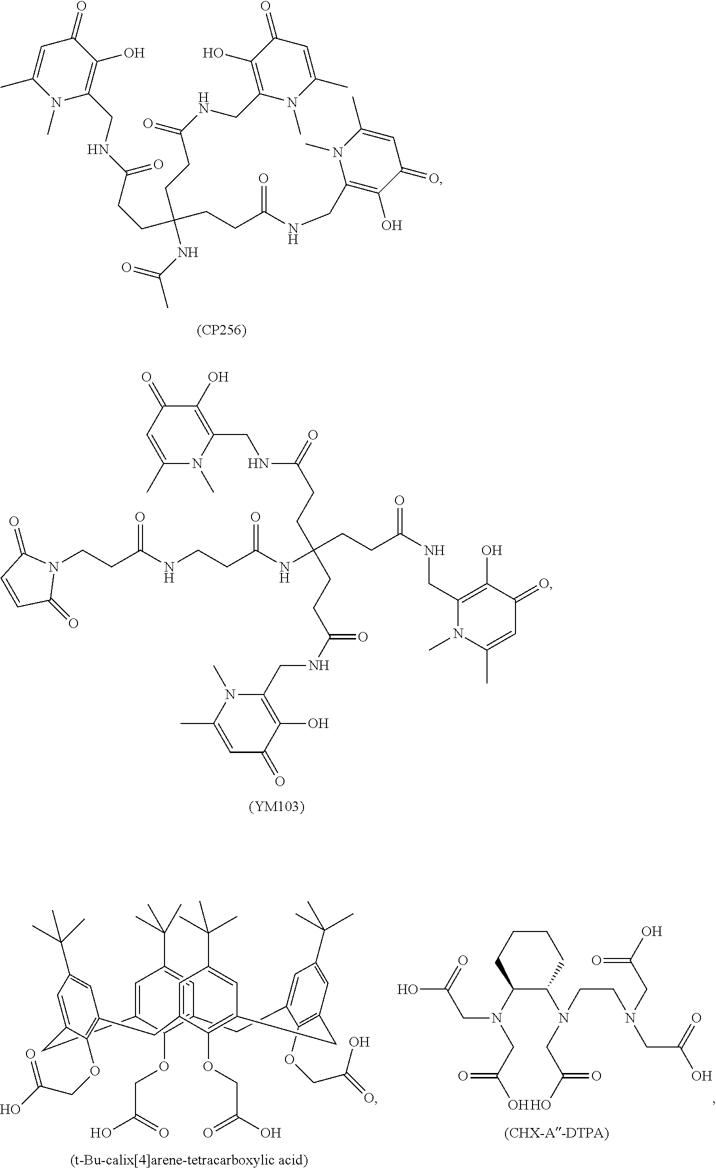

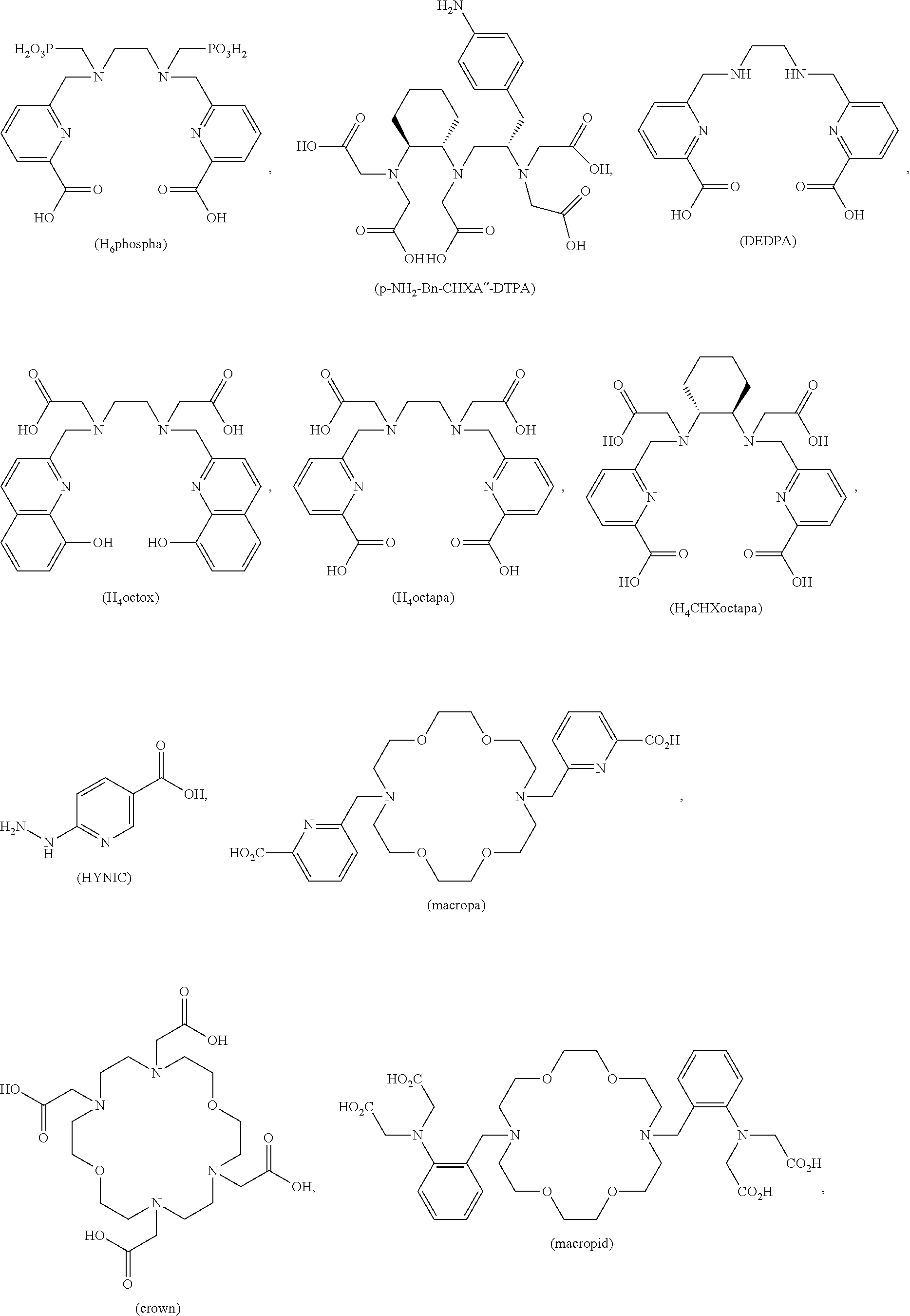

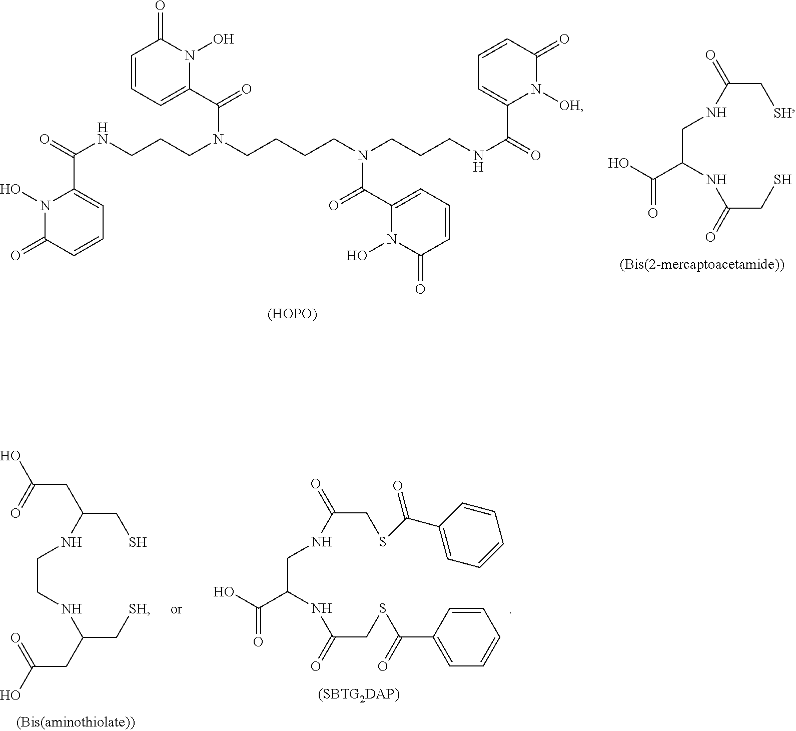 [SMILES: O=C(NCCCN(CCCCN(CCCNC(=O)c1cccc(=O)n1O)C(=O)c1cccc(=O)n1O)C(=O)c1cccc(=O)n1O)c1cccc(=O)n1O ; O=C(CS)NCC(NC(=O)CS)C(=O)O ; "(HOPO)" ; O=C(O)CC(CS)NCCNC(CS)CC(=O)O ; O=C(CSC(=O)c1ccccc1)NCC(NC(=O)CSC(=O)c1ccccc1)C(=O)O]

In some embodiments, the chelating moiety R^{a} comprises a radionuclide and DOTA. In some embodiments, the chelating moiety R^{a} comprises a radionuclide and a DOTA derivative. In some embodiments, the chelating moiety comprises two independent chelators, and at least one or both are DOTA.

In some embodiments, the chelating moiety comprises a radionuclide and a chelator configured to bind the radionuclide (Z′), wherein the chelator comprises DOTA, DOTP, DOTMA, DOTAM, DTPA, NOTA, NTA, NODAGA, EDTA, DO3A, DO2′A, NOC, TETA, CB-TE2A, DiAmSar, CB-Cyclam, DOTA-4AMP, H₄pypa, H₄octox, H₄octapa, p-NO₂—Bn-neunpa, or NOTP.

In some embodiments, the metal chelator described herein comprises macropa or crown. In some embodiments, the metal chelator described herein comprises macropa. In some embodiments, the metal chelator described herein comprises crown. In some embodiments, the metal chelator described herein comprises

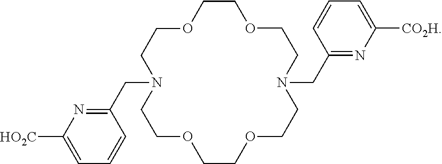 [SMILES: O=C(O)c1cccc(CN2CCOCCOCCN(Cc3cccc(C(=O)O)n3)CCOCCOCC2)n1]

(macropa). In some embodiments, the metal chelator described herein comprises

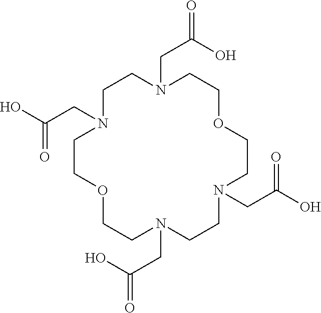 [SMILES: O=C(O)CN1CCOCCN(CC(=O)O)CCN(CC(=O)O)CCOCCN(CC(=O)O)CC1]

(crown).

In some embodiments, the chelating moiety of R^{a} is independently selected from the group consisting of: cyclen, DO2A, DO3A, HP-DO3A, DO3A-Nprop, DO3AP, DO3AP^{PrA}, DO3AP^{ABn}, DO3AM^{nBu}, BT-DO3A, DOTA, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-Aⁿ-DTPA, c-DEPA, PCTA, p-NO₂—Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂—Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), and SBTG₂DAP.

In some embodiments, R^{a} is a chelating moiety selected from the group consisting of: DOTA; 2,2′,2″-(10-(2-amino-2-oxoethyl)-1,4,7,10-tetraazacyclododecane-1,4,7-triyl)triacetic acid (PSC); DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; Bn-DOTA; p-OH-Bn-DOTA; H₄pypa; H₄pypa-benzyl; H₄py4pa; H₄py4pa-benzyl; NOTA; macropa; crown; H₄octapa; H₄octapa-benzyl; and TTHA; or a radionuclide complex thereof.

In some embodiments, the chelating moiety of R^{a} is independently selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, the chelating moiety of R^{a} is independently selected from the group consisting of: DOTA; DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; H₄pypa; H₄py4pa; NOTA; macropa; crown; H₄octapa; and TTHA; or a radionuclide complex thereof.

In some embodiments, R^{a} is DOTA or a radionuclide complex thereof. In some embodiments, R^{a} is DO3A or a radionuclide complex thereof. In some embodiments, R^{a} is DO2A or a radionuclide complex thereof. In some embodiments, R^{a} is DOTMA or a radionuclide complex thereof. In some embodiments, R^{a} is DOTAM or a radionuclide complex thereof. In some embodiments, R^{a} is DOTPA or a radionuclide complex thereof. In some embodiments, R^{a} is 2,2′,2″-(10-(2-amino-2-oxoethyl)-1,4,7,10-tetraazacyclododecane-1,4,7-triyl)triacetic acid or a radionuclide complex thereof. In some embodiments, R^{a} is H₄pypa or a radionuclide complex thereof. In some embodiments, R^{a} is H₄py4pa or a radionuclide complex thereof. In some embodiments, R^{a} is NOTA or a radionuclide complex thereof. In some embodiments, R^{a} is macropa or a radionuclide complex thereof. In some embodiments, R^{a} is crown or a radionuclide complex thereof. In some embodiments, R^{a} is H₄octapa or a radionuclide complex thereof. In some embodiments, R^{a} is TTHA or a radionuclide complex thereof.

In some embodiments, R^{a} is: DOTA or DO3A; or a radionuclide complex thereof.

In some embodiments, the chelating moiety of R^{a} is independently selected from the group consisting of: DOTA; DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; H₄pypa; H₄py4pa; NOTA; macropa; crown; H₄octapa; and TTHA; or a radionuclide complex thereof.

In some embodiments, R^{a} is DOTA or a radionuclide complex thereof. In some embodiments, R^{a} is DO3A or a radionuclide complex thereof. In some embodiments, R^{a} is DO2A or a radionuclide complex thereof. In some embodiments, R^{a} is DOTMA or a radionuclide complex thereof. In some embodiments, R^{a} is DOTAM or a radionuclide complex thereof. In some embodiments, R^{a} is DOTPA or a radionuclide complex thereof. In some embodiments, R^{a} is 2,2′,2″-(10-(2-amino-2-oxoethyl)-1,4,7,10-tetraazacyclododecane-1,4,7-triyl)triacetic acid or a radionuclide complex thereof. In some embodiments, R^{a} is H₄pypa or a radionuclide complex thereof. In some embodiments, R^{a} is H₄pypa or a radionuclide complex thereof. In some embodiments, R^{a} is NOTA. In some embodiments, R^{a} is macropa. In some embodiments, R^{a} is crown. In some embodiments, R^{a} is H₄octapa or a radionuclide complex thereof. In some embodiments, R^{a} is TTHA or a radionuclide complex thereof.

In some embodiments, the chelating moiety of R^{a} is: DOTA or DO3A; or a radionuclide complex thereof.

In some embodiments, R^{a} is a chelating moiety selected from the group consisting of:

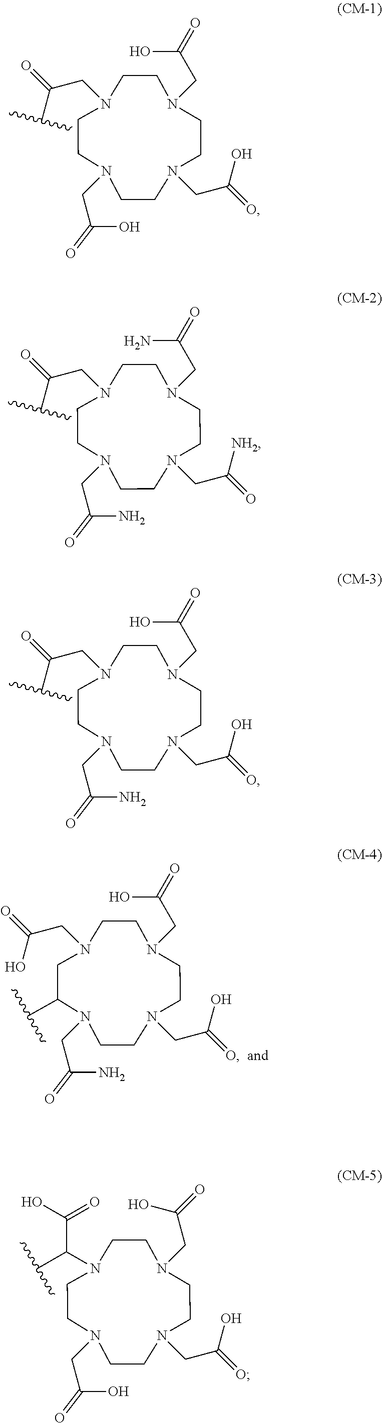

or a radionuclide complex thereof.

In some embodiments, R^{a} is a chelating moiety selected from the group consisting of: (CM-1), (CM-2), (CM-4) and (CM-5); or a radionuclide complex thereof.

In some embodiments, R^{a} is (CM-1); or a radionuclide complex thereof.

In some embodiments, R^{a} is: (CM-2), (CM-3), (CM-4), or (CM-5); or a radionuclide complex thereof.

In some embodiments, R^{a} is (CM-2), (CM-4), or (CM-5); or a radionuclide complex thereof.

In some embodiments, R^{a} is: (CM-2); or a radionuclide complex thereof. In some embodiments, R^{a} is: (CM-3); or a radionuclide complex thereof. In some embodiments, R^{a} is: (CM-5); or a radionuclide complex thereof.

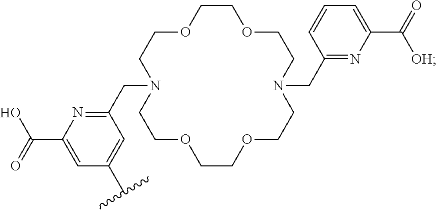 [SMILES: *c1cc(CN2CCOCCOCCN(Cc3cccc(C(=O)O)n3)CCOCCOCC2)nc(C(=O)O)c1]

In some embodiments, R^{a} is: or a radionuclide complex thereof. In some embodiments, R^{a} is

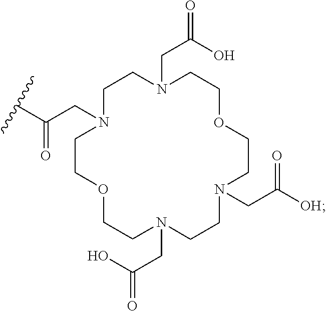 [SMILES: *C(=O)CN1CCOCCN(CC(=O)O)CCN(CC(=O)O)CCOCCN(CC(=O)O)CC1]

or a radionuclide complex thereof.

In some embodiments, R^{a} is:

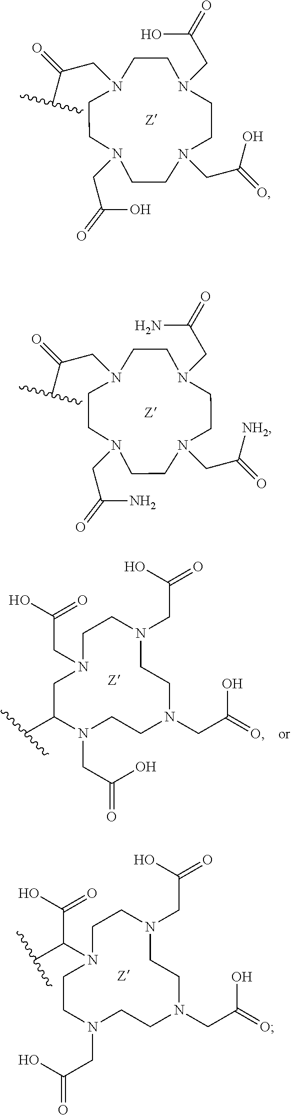

wherein Z′ is a diagnostic or therapeutic radionuclide.

In some embodiments, R^{a} is: or

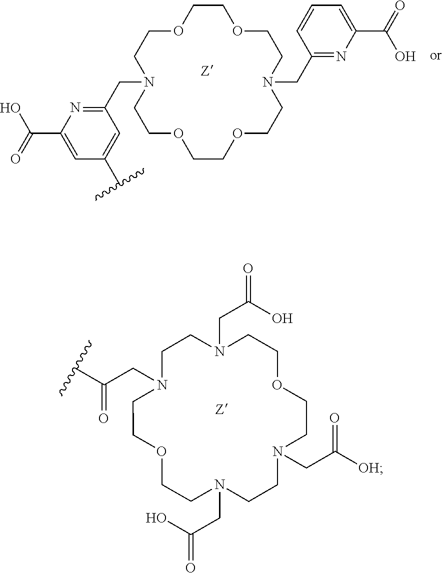

wherein Z is a diagnostic or therapeutic radionuclide.

In some embodiments, Z′ is a lanthanide or an actinide.

In some embodiments, Z′ is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

In some embodiments, Z′ is an Auger electron-emitting radionuclide, α-emitting radionuclide, β-emitting radionuclide, or γ-emitting radionuclide.

In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{19m}Pt); or an α-emitting radionuclide that is 225-actinium (²²⁵Ac), 213-bismuth (²¹³Bi), 223-Radium (²²³Ra), or 212-lead (²¹²Pb); or a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z is an Auger electron-emitting radionuclide that is 111-indium (¹¹¹In), 67-gallium (⁶⁷Ga), 68-gallium (⁶⁸Ga), 99m-technetium (^{99m}Tc), 64-copper (⁶⁴Cu), or 195m-platinum (^{195m}Pt).

In some embodiments, Z′ is an α-emitting radionuclide that is 225-actinium (²²⁵Ac), 213-bismuth (²¹³Bi), 223-radium (²²³Ra), or 212-lead (²¹²Pb). In some embodiments, Z is a -emitting radionuclide that is 90-yttrium (⁹⁰Y), 177-lutetium (¹⁷⁷Lu), iodine-131 (¹³¹I), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn).

In some embodiments, Z′ is a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, R^{a} comprises a radionuclide (Z′) and a chelator configured to bind the radionuclide (Z′), wherein the radionuclide is suitable for positron emission tomography (PET) analysis, single-photon emission computerized tomography (SPECT), or magnetic resonance imaging (MRI). In some embodiments, the radionuclide is copper-64 (⁶⁴Cu), gallium-68 (⁶⁸Ga), 111-indium (¹¹¹In), or technetium-99m (^{99m}Tc).

### Metals (Radionuclides)

In some embodiments, Z′ is an Auger electron-emitting radionuclide. In some embodiments, Z′ is an α-emitting radionuclide. In some embodiments, Z′ is a n-emitting radionuclide. In some embodiments, Z′ is a y-emitting radionuclide. In some embodiments, the type of radionuclide used in a peptide targeted therapeutic compound can be tailored to the specific type of cancer, the type of targeting moiety (e.g., peptide ligand), etc. Radionuclides that undergo α-decay emit α-particles (helium ions with a +2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles.

Auger electrons (AEs) are very low energy electrons that are emitted by radionuclides that decay by electron capture (EC) (e.g. ¹¹¹In, ⁶⁷Ga, ^{99m}Tc, ^{195m}Pt, ¹²⁵I and ¹²³I). This energy is deposited over nanometer-micrometer distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 un in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An α-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20 α-particle traversals of the cell nucleus. The resulting high potency, combined with the short range of α-particles (which reduces normal organ toxicity), has led to substantial interest in developing α-particle-emitting agents. The α-particle emitters typically used include bismuth-212, lead-212, bismuth-213. actinium-225, radium-223 and thorium-227.

In some embodiments, Z′ is a diagnostic or therapeutic radionuclide.

### Representative Radionuclides

| Isotope | Radionuclide t_{1/2} (h) | Decay mode |
|---|---|---|
| ⁶⁰Cu | 0.4 | β+ (93%), EC (7%) |
| ⁶¹Cu | 3.3 | β+ (62%), EC (38%) |
| ⁶²Cu | 0.16 | β+ (98%), EC (2%) |
| ⁶⁴Cu | 12.7 | β+ (19%), EC (41%), |
| | | β− (40%) |
| ⁶⁷Cu | 61.9 | |
| ⁶⁶Ga | 9.5 | β+ (56%), EC (44%) |
| ⁶⁷Ga | 78.2 | EC (100%) |
| ⁶⁸Ga | 1.1 | β+ (90%), EC (10%) |
| ⁴⁴Sc | 3.9 | β+ (94%), EC (6%) |
| ⁴⁷Sc | 80.2 | β− (100%) |
| ¹¹¹In | 67.2 | EC (100%) |
| ^{114m}In | 49.5 d | EC (100%) |
| ¹¹⁴In (daughter) | 73 s | β− (100%) |
| ¹⁷⁷Lu | 159.4 | β− (100%) |
| ⁸⁶Y | 14.7 | β+ (33%), EC (66%) |
| ⁹⁰Y | 64.1 | β− (100%) |
| ⁸⁹Zr | 78.5 | β+ (23%), EC (77%) |
| ²¹²Bi | 1.1 | α (36%), β− (64%) |
| ²¹³Bi | 0.76 | α (2.2%), β− (97.8%) |
| ²¹²Pb | 10.6 | β− (100%) |
| (daughter is ²¹²Bi) | | |
| ²²⁵Ac | 240 | α (100%) |
| ²²⁷Th | 448.8 | α |
| ²¹¹At | 7.2 | α |

In some embodiments, Z′ is an Auger electron-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt).

In some embodiments, Z′ is an α-emitting radionuclide. In some embodiments, Z′ is an α-emitting radionuclide that is 225-actinium (²²⁵Ac), 213-bismuth (²¹³Bi), 223-radium (²²³Ra), or 212-lead (²¹²Pb).

In some embodiments, Z′ is a β-emitting radionuclide. In some embodiments, Z′ is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (67Cu), 153-samarium (¹⁵³Sm), 89-strontium (89Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn).

In some embodiments, Z′ is a γ-emitting radionuclide. In some embodiments, Z′ is a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or Z′ is an α-emitting radionuclide that is 225-actinium (²²⁵Ac), 213-bismuth (²¹³Bi), 223-Radium (²²³Ra), or 212-lead (²¹²Pb); or Z′ is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or Z′ is a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is 90-yttrium (⁹⁰Y), 177-lutetium (177Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

In some embodiments, Z′ is ⁹⁴Tc, ⁹⁰In, ¹¹¹In, ⁶⁷Ga, ⁶⁸Ga, ⁸⁶Y, ⁹⁰Y, ¹⁷⁷Lu, ¹⁶¹Tb, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²²5Ac, ²¹³Bi, ²¹²Bi, ²¹²Pb, ²²⁷Th, ¹⁵³Sm, ¹⁶⁶Ho, ¹⁵²Gd, ¹⁵³Gd, ¹⁵⁷Gd, or ¹⁶⁶Dy.

In some embodiments, Z′ is ⁶⁷Cu, ⁶⁴Cu, ⁹⁰Y, ¹⁰⁹Pd, ¹¹Ag, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ⁴⁴Sc, ⁴⁷Se, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹Tl, ¹⁶⁰Gd, ¹⁴⁸Nd, or ⁸⁹Sr.

In some embodiments, Z′ is ⁶⁸Ga, ⁴³Se, ⁴⁴Sc, ⁴⁷Sc, ¹⁷⁷Lu, ¹⁶¹Tb, ²²⁵Ac, ²¹³Bi, ²¹²Bi, or ²¹²Pb. In some embodiments, Z′ is ⁶⁷Ga, ^{99m}Tc, ¹¹¹In, or ²⁰¹Tl.

In some embodiments, the radionuclide (Z′) is ⁴⁴Sc, ⁶⁴Cu, 67Ga, ⁶⁸Ga, ⁸⁶Y ⁸⁹Zr, ^{99m}Tc, ¹¹¹In, or ¹⁷⁷Lu.

In some embodiments, Z′ is ⁴⁴Sc, ⁶⁴Cu, ⁶⁸Ga, ⁸⁶Y, or ⁸⁹Zr. In some embodiments, Z′ is ⁶⁷Ga, ^{99m}Tc, ¹¹¹In, or ¹⁷⁷Lu.

In some embodiments, Z′ is ⁶⁷Cu, ⁹⁰Y, ¹¹¹In, ¹⁷⁷Lu, ²²⁵Ac, ²¹²Pb, or ²³¹Bi.

In some embodiments, Z′ is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²¹⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

In some embodiments, Z′ is 111-indium (¹¹¹In). In some embodiments, Z′ is 115-indium (¹¹⁵In). In some embodiments, Z′ is 67-gallium (⁶⁷Ga). In some embodiments, Z′ is 68-gallium (⁶⁸Ga). In some embodiments, Z′ is 69-gallium (⁶⁹Ga), 71-gallium (⁷¹Ga), or a mixture thereof. In some embodiments, Z′ is 225-actinium (²²⁵Ac). In some embodiments, Z′ is 175-lutetium (¹⁷⁵Lu). In some embodiments, Z′ is 177-lutetium (¹⁷⁷Lu). In some embodiments, Z′ is 204-lead (²⁰⁴Pb), 206-lead (²⁰⁶Pb), 207-lead (²⁰⁷Pb), 208-lead (²⁰⁸Pb), or a mixture thereof. In some embodiments, Z′ is 212-lead (²¹²Pb). In some embodiments, Z′ is 64-copper (⁶⁴Cu). In some embodiments, Z′ is 63-copper (⁶³Cu), 65-copper (⁶⁵Cu), or a mixture thereof. In some embodiments, Z′ is 67-copper (67Cu).

In some embodiments, Z′ is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

### Exemplary Chelator and Radionuclide Complexes

Radionuclides have useful emission properties that can be used for diagnostic imaging techniques, such as single photon emission computed tomography (SPECT, e.g. 67Ga, ^{99m}Tc, ¹¹¹In, ¹⁷⁷Lu) and positron emission tomography (PET, e.g. ⁶⁸Ga, ⁶⁴Cu, ⁴⁴Sc, ⁸⁶Y, ⁸⁹Zr), as well as therapeutic applications (e.g. ⁴⁷Sc, ¹¹⁴mIn, ¹⁷⁷Lu, ⁹⁰Y, ^{212/213}Bi, ²¹²Pb, ²²⁵Ac, ^{186/188}Re). A fundamental component of a radiometal-based radiopharmaceutical is the chelator, the ligand system that binds the radiometal ion in a tight stable coordination complex so that it can be properly directed to a desirable molecular target in vivo. Guidance for selecting the optimal match between chelator and radiometal for a particular use is provided in the art (e.g., see Price et al., “Matching chelators to radiometals for radiopharmaceuticals”, Chem. Soc. Rev., 2014, 43, 260-290).

In some embodiments, R^{a} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof.

In some embodiments, R^{a} is:

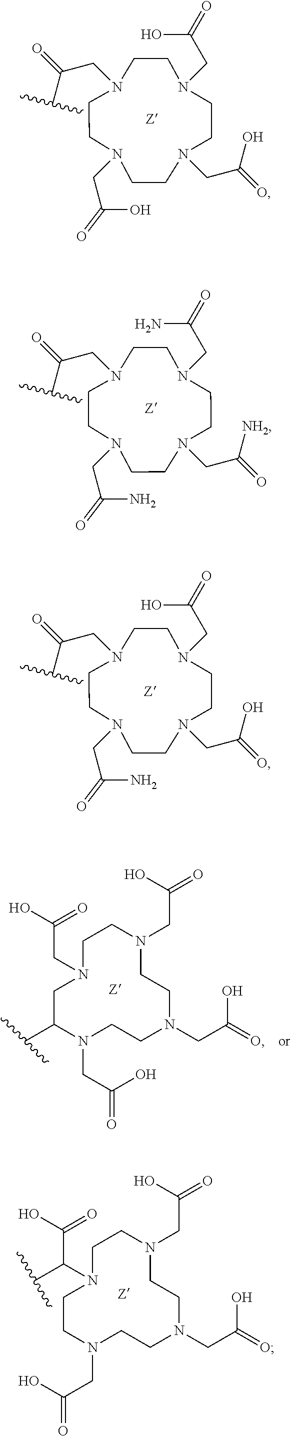

wherein Z′ is a diagnostic or therapeutic radionuclide.

In some embodiments, the radionuclide (Z′) is ⁴⁴Sc, 64Cu, ⁶⁷Ga, ⁶⁸Ga, ^{86Y}, ⁸⁹Zr, ^{99m}Tc, ¹¹¹In, or ¹⁷⁷Lu. In some embodiments, the radionuclide (Z′) is ⁴⁴Sc, ⁶⁴Cu, ⁶⁸Ga, ⁸⁶Y, or ⁸⁹Zr. In some embodiments, the radionuclide (Z′) is ⁶⁷Ga, ^{99m}Tc, ¹¹¹In, or ¹⁷⁷Lu.

In some embodiments, the radionuclide (Z′) is 67Cu, ⁹⁰Y, ¹¹¹In, 177Lu, ²²⁵Ac, ²¹²Pb, or ²¹³Bi.

In some embodiments, the radionuclide (Z′) is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (177Lu), 206-lead (²⁰⁶Pb), 207-lead (²⁰⁷Pb), 208-lead (²⁰⁸Pb), 212-lead (²¹²Pb), 60-copper (⁶⁰Cu), 61-copper (61Cu), 62-copper (⁶²Cu), 63-copper (⁶³Cu), 64-copper (⁶⁴Cu), 65-copper (⁶⁵Cu), or 67-copper (⁶⁷Cu).

In some embodiments, the radionuclide (Z′) is 111-indium (¹¹¹In) or 115-indium (¹¹⁵In), or a mixture thereof. In some embodiments, the radionuclide (Z′) is 67-gallium (⁶⁷Ga), 68-gallium (⁶⁸Ga), 69-gallium (⁶⁹Ga), or 71-gallium (⁷¹Ga), or a mixture thereof. In some embodiments, the radionuclide (Z′) is 225-actinium (²²⁵Ac). In some embodiments, the radionuclide (Z′) is 175-lutetium (¹⁷⁵Lu) or 177-lutetium (¹⁷⁷Lu), or a mixture thereof. In some embodiments, the radionuclide (Z′) is 206-lead (²⁰⁶Pb), 207-lead (²⁰⁷Pb), 208-lead (²⁰⁸Pb), or 212-lead (²¹²Pb), or a mixture thereof. In some embodiments, the radionuclide (Z′) is 60-copper (⁶⁰Cu), 61-copper (⁶¹Cu), 62-copper (⁶²Cu), 63-copper (⁶³Cu), 64-copper (⁶⁴Cu), 65-copper (⁶⁵Cu), or 67-copper (⁶⁷Cu), or a mixture thereof.

In some embodiments, the radionuclide (Z′) is 111-indium (¹¹¹In), 115-indium (¹¹⁵In), 67-gallium (⁶⁷Ga), 68-gallium (⁶⁸Ga), 225-actinium (22Ac), 175-lutetium (¹⁷⁵Lu) or 177-lutetium (¹⁷⁷Lu).

In some embodiments, the radionuclide (Z′) is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

### Emission Tomography

In some embodiments, R^{a}comprises a chelated radionuclide that is suitable for positron emission tomography (PET) analysis or single-photon emission computerized tomography (SPECT). In some embodiments, R^{a} comprises a chelated radionuclide that is suitable for single-photon emission computerized tomography (SPECT). In some embodiments, R^{a} comprises a chelated radionuclide that is suitable for positron emission tomography (PET) analysis. In some embodiments, R^{a} comprises a chelated radionuclide that is suitable for positron emission tomography imaging, positron emission tomography with computed tomography imaging, or positron emission tomography with magnetic resonance imaging (MRI).

In some embodiments, R^{a} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof. In some embodiments, the radionuclide is copper-64 (⁶⁴Cu), gallium-68 (⁶⁸Ga), or technetium-99m (^{99m}Tc).

In some embodiments, a conjugate described herein is designed to have a prescribed elimination profile. The elimination profile can be designed by adjusting the sequence and length of the peptide ligand, the property of the linker, the type of radionuclide, etc. In some embodiments, the conjugate has an elimination half-life of about 5 minutes to about 12 hours. In some embodiments, the conjugate has an elimination half-life of about 10 minutes to about 8 hours. In some embodiments, the conjugate has an elimination half-life of at least about 15 minutes, at least about 30 minutes, at least about 1 hour, at least about 2 hours, at least about 3 hours, at least about 4 hours, at least about 5 hours, at least about 6 hours, or at least about 8 hours. In some embodiments, the conjugate has an elimination half-life of at most about 15 minutes, at most about 30 minutes, at most about 1 hour, at most about 2 hours, at most about 3 hours, at most about 4 hours, at most about 5 hours, at most about 6 hours, or at most about 8 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 15 minutes to about 1 day. In some embodiments, the elimination half-life of the conjugate in a tumor is at least 1.1, at least 1.2, at least 1.3, at least 1.4, at least 1.5, at least 2.0, at least 2.5, at least 3.0, at least 4.0, or at least 5.0-fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the pharmaceutical in the living system. In some embodiments, the elimination half-life is measured as effective half-life, which is the half-life of a radiopharmaceutical in a living system taking into account the half-life of the radionuclide.

Response and toxicity prediction is essential for the rational implementation of cancer therapy. The biological effects of radionuclide therapy are mediated by a well-defined physical quantity, the absorbed dose (D), which is defined as the energy absorbed per unit mass of tissue.

Radiation dosimetry is the measurement, calculation and assessment of the ionizing radiation dose absorbed by an object, usually the human body, and may be thought of as the ability to perform the equivalent of a pharmacodynamic study in treated patients in real time. This applies both internally, due to ingested or inhaled radioactive substances, or externally due to irradiation by sources of radiation. Dosimetry analysis may be performed as part of patient treatment to calculate tumor versus normal organ absorbed dose and therefore the likelihood of treatment success.

A conjugate described herein can have a prescribed time-integrated activity coefficient (i.e., ã) in a tumor or non-tumor tissues of a subject. As used herein, ã represents the cumulative number of nuclear transformations occurring in a source tissue over a dose-integration period per unit administered activity. The ã value of a conjugate can be tuned by modifications of the NPDC. The ã value can be determined using a method known in the art. In some embodiments, the ã value of the conjugate in a tumor is from about 10 minutes to about 1 day. The ã value of the conjugate in a tumor can be the same as the ã value of the conjugate in a non-tumor tissue of the subject. The ã value of the conjugate in a tumor can be longer or shorter than the ã value of the conjugate in a non-tumor tissue of the subject. In some embodiments, the ã value of the conjugate in a tumor is at least 1.1, at least 1.2, at least 1.3, at least 1.4, at least 1.5, at least 2.0, at least 2.5, at least 3.0, at least 4.0, or at least 5.0-fold of the ã value of the conjugate in a non-tumor tissue of the subject.

A conjugate described herein can have an a value in an organ of a subject. In some embodiments, the conjugate has an a value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the ã value of the conjugate in a kidney of the subject is about 30 minutes to about 24 hours. In some embodiments, the ã value of the conjugate in a kidney of the subject is about 2 to 24 hours. In some embodiments, the ã value of the conjugate in a kidney of the subject is more than 24 hours. In some embodiments, the ã value of the conjugate in a liver of the subject is at most 24 hours. In some embodiments, the ã value of the conjugate in a liver of the subject is at most 18 hours, 15 hours, 12 hours, 10 hours, 8 hours, 6 hours, or 5 hours. In some embodiments, the ã value of the conjugate in a liver of the subject is about 30 minutes to about 24 hours. In some embodiments, the ã value of the conjugate in a liver of the subject is about 2 to 24 hours. In some embodiments, the ã value of the conjugate in a liver of the subject is more than 24 hours.

### Linkers

In some embodiments, the linker has a prescribed length thereby linking the Neurotensin Receptor 1 (NTSR1) targeting ligand and the chelating moiety or a radionuclide complex thereof (R^{a}) while allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases.

In some embodiments, a linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the linker comprises a click chemistry residue. In some embodiments, the linker is attached to a peptide ligand, to a metal chelator or both via click chemistry. For example, in some embodiments, a peptide ligand comprises an azide group that reacts with an alkyne moiety of the linker. For another example, in some embodiments, a peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

In some embodiments,
L is: -L², -L⁴-, -L²-L⁴-, -L²-L⁶-, -L³-L⁶-, -L²-L³-L⁴-, -L²-L³-L⁶-, -L²-L⁵-L⁶-, -L²-L³-L⁵-L⁶- or -L²-L³-L⁴-L⁵-L⁶-;
L² is absent, —C₁-C₂₀ alkylene-, —C₁-C₂₀ alkylene-NR¹³—, —C₁-C₂₀ alkylene-NR¹³—C(═O)—, —NR¹³—C₁-C₂₀ alkylene-, —NR¹³—C₁-C₂₀ alkylene-NR¹⁶—, —C₁-C₂₀ alkylene-O—, —O—C₁-C₂₀ alkylene-, —O—C₁-C₂₀ alkylene-NR⁶—, —NR¹³—C(═O)—C₁-C₂₀ alkylene-NR¹⁶—, —C₁-C₂₀ alkylene-C(═O)—, —C(═O)—C₁-C₂₀ alkylene-, —C(═O)—C₁-C₂₀ alkylene-NR¹³—, —C(═O)—NR¹³—C₁-C₂₀ alkylene-NR¹⁶—, —C(═NH)NH—C₁-C₂₀ alkylene-NR¹³—, —C(═NH)NH—C₁-C₂₀ alkylene-, or —C(═O)—NH—O—(CH₂)ᵥ—; wherein each C₁-C₂₀ alkylene is optionally substituted with 1-3 substituents selected from the group consisting of —OH, —NH₂, and —COOH;
   each R¹³ and R¹⁶ are independently selected from H or —C₁-C₄ alkyl; each v is independently 1, 2, 3, 4, 5 or 6;
L³ is absent, —C₁-C₂₀ alkylene-, —C₁-C₂₀ alkylene-NR¹⁴—, —C(═O)—C₁-C₂₀ alkylene-, —(CH₂CH₂O)_{w}—CH₂CH₂—, —C(═O)—(CH₂CH₂O)_{w}—CH₂CH₂—, —(CH₂CH₂NR¹⁴)_{w}—CH₂CH₂—, —C(═O)NR¹⁴—(CH₂CH₂NR¹⁴)_{w}—CH₂CH₂—, —C(═O)NH—O—(CH₂)—, or —C(═O)—(C₁)_{w}—O—NH—(CH₂)_{w}—C(═O)—; wherein each C₁-C₂₀ alkylene is optionally substituted with 1-3 substituents selected from the group consisting of —OH and —NH₂;
   each R⁴ is independently H or —C₁-C₆ alkyl;
   each w is independently 1, 2, 3, 4, 5 or 6;
L⁴ is absent, a natural or unnatural amino acid, or a peptide that is formed from two or more independently selected natural and unnatural amino acids, wherein when two or more amino acids are present then the N atom of the amide linking the amino acids is optionally substituted with —C₁-C₆ alkyl;
L⁵ is absent or -L⁷-L⁸-L⁹-;
   L⁷ is absent, —O—, —S—, —NH— or —NCH₃—;
   L⁸ is substituted or unsubstituted cycloalkylene, substituted or unsubstituted cycloalkenylene, or substituted or unsubstituted heterocycloalkylene;
   L⁹ is absent, —(CH₂)ₓ—, —C(═O)(CH₂)ₓ—, or —NR¹⁵(CH₂)ₓ—;
      x is 1, 2, 3, 4, 5 or 6;
   each R¹⁵ is independently selected from H or —C₁-C₆ alkyl; and
L⁶ is absent, —NH—, or —N(CH₃)—;
wherein if L⁶ is absent, L⁴ cannot be absent.

In some embodiments,
L is: -L², -L⁴-, -L²-L³-, -L²-L⁴-, -L²-L⁶-, -L³-L⁶-, -L²-L³-L⁴-, -L²-L³-L⁶-, -L²-L⁵-L⁶-, - L²-L³-L⁵-L⁶- or -L²-L³-L⁴-L⁵-L⁶-;
L² is absent, —C₁-C₂₀ alkylene-, —C₁-C₂₀ alkylene-NR¹³—, —C₁-C₂₀ alkylene-NR¹³—C(═O)—, —NR¹³—C₁-C₂₀ alkylene-, —NR¹³—C₁-C₂₀ alkylene-NR¹⁶—, —C₁-C₂₀ alkylene-O—, —O—C₁-C₂₀ alkylene-, —O—C₁-C₂₀ alkylene-NR¹⁶—, —NR¹³—C(═O)—C₁-C₂₀ alkylene-NR¹⁶—, —C₁-C₂₀ alkylene-C(═O)—, —C(═O)—C₁-C₂₀ alkylene-, —C(═O)—C₁-C₂₀ alkylene-NR¹³—, —C(═O)—NR¹³—C₁-C₂₀ alkylene-NR¹⁶—, —C(═NH)NH—C₁-C₂₀ alkylene-NR¹³—, —C(═NH)NH—C₁-C₂₀ alkylene-, or —C(═O)—NH—O—(CH₂)ᵥ—; wherein each C₁-C₂₀ alkylene is optionally substituted with 1-3 substituents selected from the group consisting of —OH and —N₂;
   each 1R¹³ and R¹⁶ are independently selected from H or —C₁-C₄ alkyl;
   each v is independently 1, 2, 3, 4, 5 or 6;
L³ is absent, —C₁-C₂₀ alkylene-, —C₁-C₂₀ alkylene-NR¹⁴—, —C(═O)—C₁-C₂₀ alkylene-, —(CH₂CH₂O)_{w}—CH₂CH₂-, —C(═O)—(CH₂CH₂O)_{w}—CH₂CH₂—, —(CH₂CH₂NR¹⁴)_{w}—CH₂CH₂—, —C(═O)NR¹⁴—(CH₂CH₂NR¹⁴)_{w}—CH₂CH₂—, —C(═O)—NH—O—(CH₂)_{w}—, or —C(═O)—(CH₂)_{w}—O—NH—(CH₂)_{w}—C(═O)—; wherein each C₁-C₂₀ alkylene is optionally substituted with 1-3 substituents selected from the group consisting of —OH and —NH₂;
   each R⁴ is independently H or —C₁-C₆ alkyl;
   each w is independently 1, 2, 3, 4, 5 or 6;
L⁴ is absent, a natural or unnatural amino acid, or a peptide that is formed from two or more independently selected natural and unnatural amino acids, wherein when two or more amino acids are present then the N atom of the amide linking the amino acids is optionally substituted with —C₁-C₆ alkyl;
L⁵ is absent or -L⁷-L⁸-L⁹-;
   L⁷ is absent, —O—, —S—, —NH— or —NCH₃—;
   L⁸ is substituted or unsubstituted cycloalkylene, substituted or unsubstituted cycloalkenylene, or substituted or unsubstituted heterocycloalkylene;
   L⁹ is absent, —(CH₂)ₓ—, —C(═O)(CH₂)ₓ—, or —NR¹⁵(CH₂)ₓ—;
      x is 1, 2, 3, 4, 5 or 6;
   each R¹⁵ is independently selected from H or —C₁-C₆ alkyl; and
L⁶ is absent, —NH—, or —N(CH₃)—;
wherein if L⁶ is absent, L⁴ cannot be absent.

In some embodiments,
L is: -L⁴-, -L²-L⁴-, -L²-L⁶-, -L²-L³-L⁴-, -L²-L³-L⁶-, -L²-L⁵-L⁶-, -L²-L³-L⁵-L⁶- or -L²-L³-L⁴-L⁵-L⁶-;
L² is absent, —C₁-C₂₀ alkylene-, —C₁-C₂₀ alkylene-NR¹³—, —C₁-C₂₀ alkylene-O—, —C₁-C₂₀ alkylene-C(═O)—, —C(═O)—C₁-C₂₀ alkylene-, —C(═O)—C₁-C₂₀ alkylene-NR¹³—, —C(═NH)NH—C₁-C₂₀ alkylene-NR¹³—, —C(═NH))NH—C₁-C₂₀ alkylene-, or —C(═O)—NH—O—(CH₂′)—; wherein each C₁-C₂ alkylene is optionally substituted with 1-3 substituents selected from the group consisting of —OH and —NH₂;
   each R¹³ is independently selected from H or —C₁-C₄ alkyl;
   each v is independently 1, 2, 3, 4, 5 or 6;
L³ is absent, —C₁-C₂₀ alkylene-, —C₁-C₂₀ alkylene-NR¹⁴—, —C(═O)—C₁-C₂₀ alkylene-, —(CH₂CH₂O)_{w}—CH₂CH₂—, —C(═O)—(CH₂CH₂O)_{w}—CH₂CH₂—, —(CH₂CH₂NR¹⁴)_{w}—CH₂CH₂—, —C(═O)NR¹⁴—(CH₂CH₂NR¹⁴)_{w}—CH₂CH₂—, —C(═O)—NH—O—(CH₂)_{w}—, or —C(═O)—(C₂)_{w}—O—NH—(CH₂)_{w}—C(═O)—; wherein each C₁-C₂₀ alkylene is optionally substituted with 1-3 substituents selected from the group consisting of —OH and —NH₂;
   each R⁴ is independently H or —C₁-C₆ alkyl;
   each w is independently 1, 2, 3, 4, 5 or 6;
L⁴ is absent, a natural or unnatural amino acid, or a peptide that is formed from two or more independently selected natural and unnatural amino acids, wherein when two or more amino acids are present then the N atom of the amide linking the amino acids is optionally substituted with —C₁-C₆ alkyl;
L⁵ is absent or -L⁷-L⁸-L⁹-;
   L⁷ is absent, —O—, —S—, —NH— or —NCH₃—;
   L⁸ is substituted or unsubstituted cycloalkylene, substituted or unsubstituted cycloalkenylene, or substituted or unsubstituted heterocycloalkylene;
   L⁹ is absent, —(CH₂)ₓ—, —C(═O)(CH₂)ₓ—, or —NR¹⁵(CH₂)ₓ—;
      x is 1, 2, 3, 4, 5 or 6;
   each R¹⁵ is independently selected from H or —C₁-C₆ alkyl; and
L⁶ is absent, —NH—, or —N(CH₃)—;
wherein if L⁶ is absent, L⁴ cannot be absent.

In some embodiments, L is: -L², -L⁴-, -L²-L³-, -L²-L⁴-, -L²-L⁶-, -L³-L⁶-, -L²-L³-L⁴-, -L²-L³-L⁶-, -L²-L⁵-L⁶-, -L²-L³-L⁵-L⁶-. In some embodiments, L is: -L², -L²-L⁴-, -L²-L⁶-, -L²-L³-L⁴, or -L²-L³-L⁶-. In some embodiments, L is: -L², -L²-L³-, -L²-L⁶-, or -L²-L³-L⁶-.

In some embodiments, L is: -L⁴-*, -L²-L⁴-*, -L²-L⁶-*, -L²-L³-L⁴-*, -L²-L³-L⁶-*, -L)-L⁵-L⁶-*, -L²-L³-L⁵-L⁶-* or -L²-L³-L⁴-L⁵-L⁶-*; wherein * denotes the attachment point of L to R^{a}. In some embodiments, L is: -L²-*, -L⁴-*, -L²-L³-*, -L²-L⁴-*, -L²-L⁶-*, -L³-L⁶-*, -L²-L³-L⁴-*, -L²-L³-L⁶-*, -L²-L⁵-L⁶*, -L²-L³-L⁵-L⁶-* or -L²-L³-L⁴-L⁵-L⁶-*; wherein * denotes the attachment point of L to R^{a}. In some embodiments, L is: -L²-*, -L²-L⁴-*, -L²-L⁶-*, -L²-L³-L⁴-*, or -L²-L³-L⁶-*; wherein * denotes the attachment point of L to R^{a}. In some embodiments, L is: -L²-*, -L²-L³-*, -L²-L⁶-*, or -L²-L³-L⁶-*; wherein * denotes the attachment point of L to R^{a}.

In some embodiments, L is: -L²-. In some embodiments, L is: -L⁴-. In some embodiments, L is -L²-L⁴-. In some embodiments, L is -L²-L⁶-. In some embodiments, L is -L²-L³-L⁴-. In some embodiments, L is -L²-L³-L⁶-. In some embodiments, L is -L²-L⁵-L⁶-. In some embodiments, L is: -L²-L³-L⁵-L⁶. In some embodiments, L is -L²-L³-L⁴-L⁵-L⁶-.

In some embodiments, L is: -L⁴-*; wherein * denotes the attachment point of L to R^{a}. In some embodiments, L is -L²-L⁴-*; wherein * denotes the attachment point of L to R^{a}. In some embodiments, L is -L²-L⁶-*; wherein * denotes the attachment point of L to R^{a}. In some embodiments, L is -L²-L³-L⁴-*; wherein * denotes the attachment point of L to R^{a}. In some embodiments, L is -L²-L³-L⁶-*; wherein * denotes the attachment point of L to R^{a}. In some embodiments, L is -L²-L⁵-L⁶-*; wherein * denotes the attachment point of L to R^{a}. In some embodiments, L is: -L²-L³-L⁵-L⁶-*; wherein * denotes the attachment point of 1. to R^{a}. In some embodiments, L is -L²-L³-L⁴-L⁵-L⁶-*; wherein * denotes the attachment point of L to R^{a}.

In some embodiments, L² is absent, —C₁-C₆ alkylene, —C₁-C₆ alkylene-NR¹³—, —C₁-C₂₀ alkylene-NR¹³—C(═O)—, —NR¹³—C₁-C₂₀ alkylene-, —NR¹³—C₁-C₂₀ alkylene-NR¹⁶—, —C₁-C₆ alkylene-C(═O)—, —C(═O)—C₁-C₆ alkylene-, —C(═O)—C₁-C₆ alkylene-NR¹³—, —C(═O)—NR¹³—C₁-C₂n alkylene-NR¹⁶-, or —C(═NH)NH—C₁-C₂₀ alkylene-NR¹³—, wherein each C₁-C₆ alkylene is optionally substituted with 1 —OH, —NH₂, and —COOH.

In some embodiments, L² is absent, —C₁-C₆ alkylene, —C₁-C₆ alkylene-NR³—, —C₁-C₆ alkylene-C(═O)—, —C(═O)—C₁-C₆ alkylene-, —C(═O)—C₁-C₆ alkylene-NR¹³—, or —C(═NH)NH—C₁-C₂₀ alkylene-NR¹³—, wherein each —C₁-C₆ alkylene is optionally substituted with 1 —OH or —NH₂. In some embodiments, L² is absent, —C₁-C₆ alkylene, —C₁-C₆ alkylene-NR¹³—, —C₁-C₆ alkylene-C(═O)—, —C(═O)—C₁-C₆ alkylene-, or —C(═O)—C₁-C₆ alkylene-NR¹³—, wherein each —C₁-C₆ alkylene is optionally substituted with 1 —OH or —NH₂.

In some embodiments, L² is absent. In some embodiments, L² is —C₁-C₂₀ alkylene, wherein C₁-C₂₀alkylene is optionally substituted with 1-3 substituents selected from the group consisting of —OH and —NH₂. In some embodiments, L² is —C₁-C₂₀ alkylene-NR¹³—, wherein C₁-C₂₀ alkylene is optionally substituted with 1-3 substituents selected from the group consisting of —OH and —NH₂. In some embodiments, L² is —C₁-C₂₀ alkylene-O—, wherein C₁-C₂₀ alkylene is optionally substituted with 1-3 substituents selected from the group consisting of —OH and —NH₂. In some embodiments, L² is —C₁-C₂₀ alkylene-C(═O)—, wherein C₁-C₂₀ alkylene is optionally substituted with 1-3 substituents selected from the group consisting of —OH and —NH₂. In some embodiments, L² is —C(═O)—C₁-C₂₀ alkylene-, wherein C₁-C₂₀ alkylene is optionally substituted with 1-3 substituents selected from the group consisting of —OH and —NH₂. In some embodiments, L² is —C(═O)—C₁-C₂₀ alkylene-NR¹³—, wherein C₁-C₂₀ alkylene is optionally substituted with 1-3 substituents selected from the group consisting of —OH and —N₂. In some embodiments, L² is —C(═NH)NH—C₁-C₂₀ alkylene-NR¹³—, wherein C₁-C₂₀ alkylene is optionally substituted with 1-3 substituents selected from the group consisting of —OH and —NH₂. In some embodiments, L² is —C(═NH)NH—C₁-C₂₀ alkylene-, wherein C₁-C₂₀ alkylene is optionally substituted with 1-3 substituents selected from the group consisting of —OH and —NH₂. In some embodiments, L² is —C(═O)—NH—O—(CH₂)ᵥ—;

In some embodiments, L² is —C₁-C₂₀ alkylene, wherein C₁-C₂₀ alkylene is optionally substituted with 1-3 substituents selected from the group consisting of —OH, —NH₂, and —COOH. In some embodiments, L² is —C₁-C₂₀ alkylene-NR¹³—C(═O)—, wherein C₁-C₂₀ alkylene is optionally substituted with 1-3 substituents selected from the group consisting of —OH, —NH₂, and —COOH. In some embodiments, L² is —NR¹³—C₁-C₂₀ alkylene-, wherein C₁-C₂₀ alkylene is optionally substituted with 1-3 substituents selected from the group consisting of —OH, —NH—, and —COOH. In some embodiments, L² is —NR¹³—C₁-C₂₀ alkylene-NR¹⁶—, wherein C₁-C₂₀ alkylene is optionally substituted with 1-3 substituents selected from the group consisting of —OH, —NH₂, and —COOH. In some embodiments, L² is —C(═O)—NR¹³—C₁-C₂₀ alkylene-NR¹⁶—, wherein C₁-C₂₀ alkylene is optionally substituted with 1-3 substituents selected from the group consisting of —OH, —NH₂, and —COOH.

In some embodiments, L² is —C₁-C₂₀ alkylene or —C₁-C₆ alkylene-NR¹³—, wherein R¹³ is H or —CH₃. In some embodiments, L² is —C₁-C₂₀ alkylene.

In some embodiments, L² is —C₁-C₆ alkylene-NR¹³—, wherein R¹³ is H or —CH₃.

In some embodiments, L³ is absent. In some embodiments, L³ is —C₁-C₂₀ alkylene-, wherein C₁-C₂₀alkylene is optionally substituted with 1-3 substituents selected from the group consisting of —OH and —NH₂. In some embodiments, L³ is —C₁-C₂₀ alkylene-NR¹⁴—, wherein C₁-C₂₀ alkylene is optionally substituted with 1-3 substituents selected from the group consisting of —OH and —NH₂. In some embodiments, L³ is —C(═O)—C₁-C₂₀ alkylene-, wherein C₁-C₂₀ alkylene is optionally substituted with 1-3 substituents selected from the group consisting of —OH and —NH₂. In some embodiments, L³ is —C₁-C₆ alkylene-, wherein C₁-C₆ alkylene is optionally substituted with 1-3 substituents selected from the group consisting of —OH and —NH₂. In some embodiments, L³ is —C₁-C₆ alkylene-NR¹⁴—, wherein C₁-C₆ alkylene is optionally substituted with 1-3 substituents selected from the group consisting of —OH and —NH₂. In some embodiments, L³ is —C(═O)—C₁-C₆ alkylene-, wherein C₁-C₆ alkylene is optionally substituted with 1-3 substituents selected from the group consisting of —OH and —NH₂. In some embodiments, L³ is —(CH₂CH₂O)_{w}—CH₂CH₂—. In some embodiments, L³ is —C(═O)—(CH₂CH₂O)_{w}—CH₂CH₂—. In some embodiments, L³ is —(CH₂CH₂NR¹⁴)—CH₂CH₂—, —C(═O)NR¹⁴—(CH₂CH₂NR¹⁴)_{w}—CH₂CH₂—. In some embodiments, L³ is —C(═O)—NH—O—(CH₂)_{w}—. In some embodiments, L³ is —C(═O)—(CH₂)_{w}—O—NH—(CH₂)_{w}—C(═O)—.

In some embodiments, L³ is —C₁-C₆ alkylene-, —C₁-C₆ alkylene-NR¹⁴—, or —C(═O)—C₁-C₆ alkylene-, wherein the C₁-C₆ alkylene is optionally substituted with 1 —OH or —NH₂.

In some embodiments, L⁴ is —C₁-C₆ alkylene- or —C(═O)—C₁-C₆alkylene-, wherein the C₁-C₆ alkylene is optionally substituted with 1 —OH or —NH₂.

In some embodiments, L⁴ is absent. In some embodiments, L⁴ is a natural amino acid, an unnatural amino acid, or peptide that is formed from two or more independently selected amino acids selected from the group consisting of alanine (Ala), 3-aminoalanine (2,3-diaminopropionic acid) (Dap), arginine (Arg), asparagine (Asn), aspartate (Asp), biphenylalanine (Bip), cysteine (Cys), cysteic acid (sulfoalanine), glutamine (Gln), glutamate (Glu), homoglutamic acid (Aad), aminopimelic acid (Apm), glycine (Gly), histidine (His), isoleucine (Ile), leucine (Leu), lysine (Lys), hydroxylysine (Hyl), ornithine (Orn), methionine (Met), phenylalanine (Phe), proline (Pro), hydroxyproline (Hyp), serine (Ser), homoserine (Hse), sarcosine (Sar), threonine (Thr), tryptophan (Trp), tyrosine (Tyr), and valine (Val); wherein when L⁴ is 3-aminoalanine, lysine (Lys), or ornithine (Orn), the N atom which is not part of the linker backbone can be substituted with

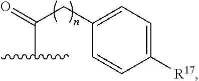

R wherein R¹⁷ is —CH₃ or —I and n is 1, 2, or 3; and wherein when L⁴ is aspartic acid or glutamic acid, the carbon atom of the carboxyl side chain can be substituted with

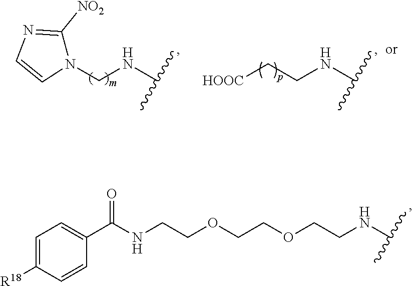

wherein m is 1, 2, or 3; p is 1, 2, 3, 4, 5, 6, 7, or 8; and R¹⁸ is —CH₃, —OCH₃, or —I; and wherein when two or more amino acids are present then the N atom of the amide linking the amino acids is optionally substituted with —CH₃.

In some embodiments, L⁴ is a natural amino acid, an unnatural amino acid, or peptide that is formed from two or more independently selected amino acids selected from the group consisting of alanine (Ala), 3-aminoalanine(2,3-diaminopropionic acid), arginine (Arg), asparagine (Asn), aspartate (Asp), biphenylalanine (Bip), cysteine (Cys), cysteic acid (sulfoalanine), glutamine (Gln), glutamate (Glu), glycine (Gly), histidine (His), isoleucine (Ile), leucine (Leu), lysine (Lys), hydroxylysine (Hyl), ornithine (Grn), methionine (Met), phenylalanine (Phe), proline (Pro), hydroxyproline (Hyp), serine (Ser), homoserine (Hse), sarcosine (Sar), threonine (Thr), tryptophan (Trp), tyrosine (Tyr), and valine (Val); wherein when L⁴ is 3-aminoalanine, lysine (Lys), or ornithine (0r), the N atom which is not part of the linker backbone can be substituted with

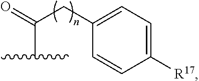

wherein R¹⁷ is —CH₃ or —I and n is 1, 2, or 3; and wherein when L⁴ is aspartic acid or glutamic acid, the carbon atom of the carboxyl side chain can be substituted with

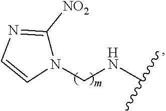

wherein m is 1, 2, or 3; and wherein when two or more amino acids are present then the N atom of the amide linking the amino acids is optionally substituted with —CH₃.

In some embodiments, L¹ is a natural amino acid, an unnatural amino acid, or peptide that is formed from two or more independently selected amino acids selected from the group consisting of alanine (Ala), 3-aminoalanine (2,3-diaminopropionic acid), arginine (Arg), asparagine (Asn), aspartate (Asp), cysteine (Cys), cysteic acid (sulfoalanine), glutamine (Gln), glutamate (Glu), glycine (Gly), histidine (His), isoleucine (Ile), leucine (Leu), lysine (Lys), hydroxylysine (Hyl), ornithine (Orn), methionine (Met), phenylalanine (Phe), proline (Pro), hydroxyproline (Hyp), serine (Ser), homoserine (Hse), sarcosine (Sar), threonine (Thr), tryptophan (Trp), tyrosine (Tyr), and valine (Val), wherein when two or more amino acids are present then the N atom of the amide linking the amino acids is optionally substituted with —CH₃.

In some embodiments, L⁴ is ornithine, lysine, glutamic acid, biphenylalanine (Bip), cysteic acid (sulfo-D-alanine), 3-amino alanine (2,3-diaminopropionic acid) (Dap), sarcosine, (4-(p-tolyl)butanoyl)-D-lysine, N5-(3-(2-nitro-1H-imidazol-1-yl)propyl)-L-glutamine, homoglutamic acid (Aad) or aminopimelic acid (Apm)

In some embodiments, L⁴ is ornithine, lysine, glutamic acid, cysteic acid (sulfo-D-alanine), 3-amino alanine (2,3-diaminopropionic acid), sarcosine, (4-(p-tolyl)butanoyl)-D-lysine, or N5-(3-(2-nitro-1H-imidazol-1-yl)propyl)-L-glutamine. In some embodiments, L⁴ is ornithine, lysine, glutamic acid, cysteic acid (sulfo-D-alanine), 3-amino alanine (2,3-diaminopropionic acid), or sarcosine.

In some embodiments, L⁴ is Sar-Sar-Sar.

In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8.

In some embodiments, R¹⁷ is —CH₃ or —I. In some embodiments, R¹⁷ is —CH₃. In some embodiments, R¹⁷ is —I.

In some embodiments, R¹⁸ is —CH₃, —OCH₃, or —I. In some embodiments, R¹⁸ is —CH₃. In some embodiments, R¹⁸ is —OCH₃. In some embodiments, R¹⁸ is —I.

In some embodiments, L⁵ is absent.

In some embodiments, L⁵ is -L⁷-L⁸-L⁹-.

In some embodiments, L⁵ is -L⁷-L⁸-L⁹-; IT is absent, —NH—, or —NCH₃—; L⁸ is substituted or unsubstituted cycloalkenylene, or substituted or unsubstituted heterocycloalkylene; L⁹ is absent, —C(═O)(CH₂)ₓ—, or —NR⁵(CH₂)ₓ—; x is 1, 2, 3, 4, 5 or 6; and each R¹⁵ is independently selected from H or —CH₃.

In some embodiments, L⁷ is absent, —O—, —S—, —NH— or —NCH₃—. In some embodiments, L⁷ is absent. In some embodiments, L⁷ is —O—. In some embodiments, L⁷ is —S—. In some embodiments, L⁷ is —NH—. In some embodiments, L⁷ is —NCH₃—.

In some embodiments, L⁸ is substituted or unsubstituted cycloalkylene. In some embodiments, L⁸ is substituted or unsubstituted cycloalkenylene. In some embodiments, L⁸ is substituted or unsubstituted heterocycloalkylene. In some embodiments, L⁸ is unsubstituted cycloalkylene. In some embodiments, L⁸ is unsubstituted cycloalkenylene. In some embodiments, L⁸ is unsubstituted heterocycloalkylene.

In some embodiments, L⁸ is

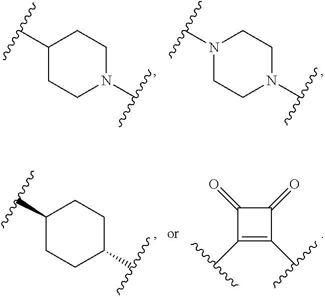 [SMILES: *C1CCN(*)CC1 ; *N1CCN(*)CC1 ; *[C@H]1CC[C@H](*)CC1 ; *c1c(*)c(=O)c1=O]

In some embodiments, L⁸ is

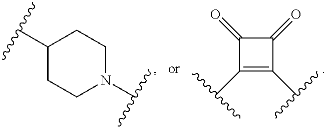 [SMILES: *C1CCN(*)CC1 ; *c1c(*)c(=O)c1=O]

In some embodiments, L⁸ is

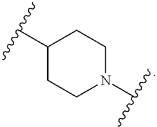 [SMILES: *C1CCN(*)CC1]

In some embodiments, L⁸ is

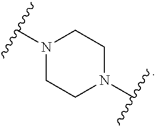 [SMILES: *N1CCN(*)CC1]

In some embodiments, L⁸ is

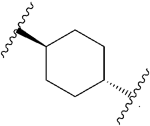 [SMILES: *[C@H]1CC[C@H](*)CC1]

In some embodiments, L⁸ is

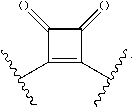 [SMILES: *c1c(*)c(=O)c1=O]

In some embodiments, L⁹ is absent, —(CH₂)ₓ—, —C(═O)(CH₂)ₓ—, or —NR¹⁵(CH₂)ₓ—. In some embodiments, L⁹ is absent. In some embodiments, L⁹ is —(CH₂)ₓ—. In some embodiments, L⁹ is —C(═O)(CH₂)ₓ—. In some embodiments, L⁹ is —NR¹⁵(CH₂)ₓ—.

In some embodiments, L⁵ is

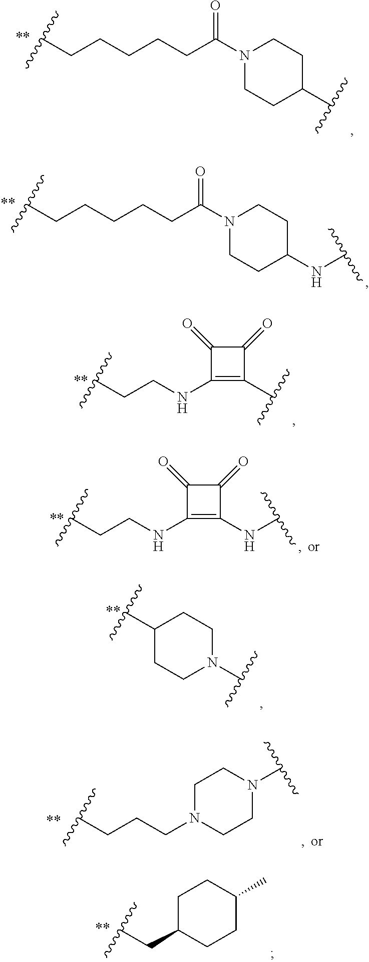

wherein denotes the attachment point of L⁵ to L⁶. In some embodiments, L⁵ is

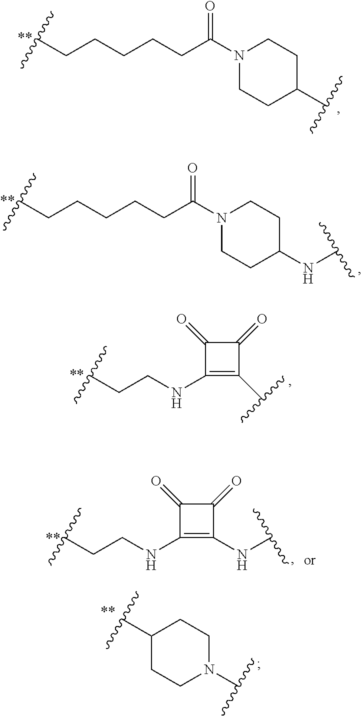 [SMILES: *CCCCCC(=O)N1CCC(*)CC1 ; *CCCCCC(=O)N1CCC(N*)CC1 ; *CCNc1c(*)c(=O)c1=O ; *CCNc1c(N*)c(=O)c1=O ; *C1CCN(*)CC1]

wherein ** denotes the attachment point of L⁵ to L⁶. In some embodiments, L⁵ is

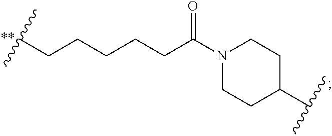 [SMILES: *CCCCCC(=O)N1CCC(*)CC1]

wherein ** denotes the attachment point of L⁵ to L⁶. In some embodiments, L⁵ is

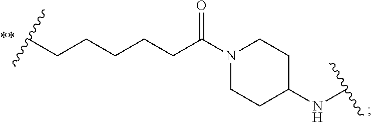 [SMILES: *CCCCCC(=O)N1CCC(N*)CC1]

wherein ** denotes the attachment point of L⁵ to L⁶. In some embodiments, L⁵ is

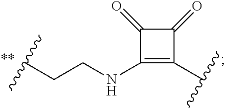 [SMILES: *CCNc1c(*)c(=O)c1=O]

wherein ** denotes the attachment point of L⁵ to L⁶. In some embodiments, L⁵ is

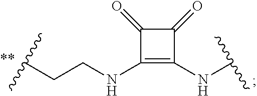 [SMILES: *CCNc1c(N*)c(=O)c1=O]

wherein ** denotes the attachment point of L⁵ to L⁶. In some embodiments, L⁵ is

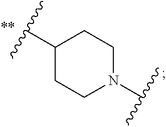 [SMILES: *C1CCN(*)CC1]

wherein ** denotes the attachment point of L⁵ to L⁶. In some embodiments, L⁵ is

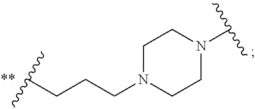 [SMILES: *CCCN1CCN(*)CC1]

wherein ** denotes the attachment point of L⁵ to L⁶. In some embodiments, L⁵ is

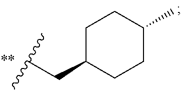 [SMILES: *C[C@H]1CC[C@H](C)CC1]

wherein ** denotes the attachment point of L⁵ to L⁶.

In some embodiments, L⁶ is —NH— or —N(CJ-3)-. In some embodiments, L⁶ is —NH—. In some embodiments, L⁶ is —N(CH₃)—.

In some embodiments, v is 1. In some embodiments, v is 2. In some embodiments, v is 3. In some embodiments, v is 4. In some embodiments, v is 5. In some embodiments, v is 6.

In some embodiments, w is 1. In some embodiments, w is 2. In some embodiments, w is 3. In some embodiments, w is 4. In some embodiments, w is 5. In some embodiments, w is 6.

In some embodiments, x is 1. In some embodiments, x is 2. In some embodiments, x is 3. In some embodiments, x is 4. In some embodiments, x is 5. In some embodiments, x is 6.

In some embodiments, R^{a}-L- is:

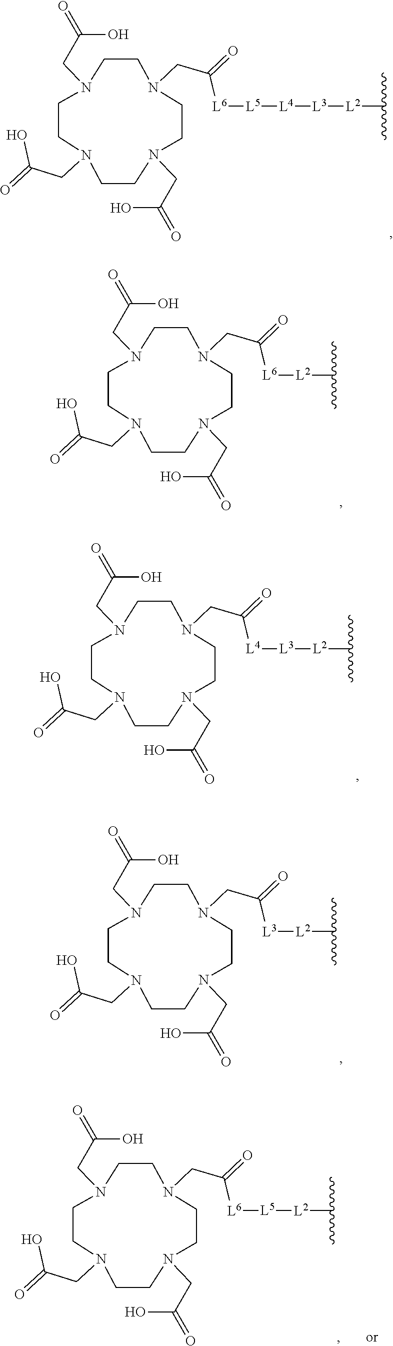

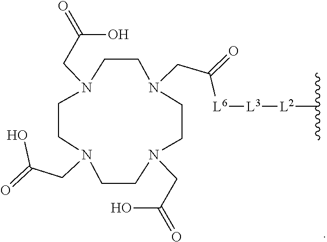

In some embodiments, L of R^{a}-L- is:

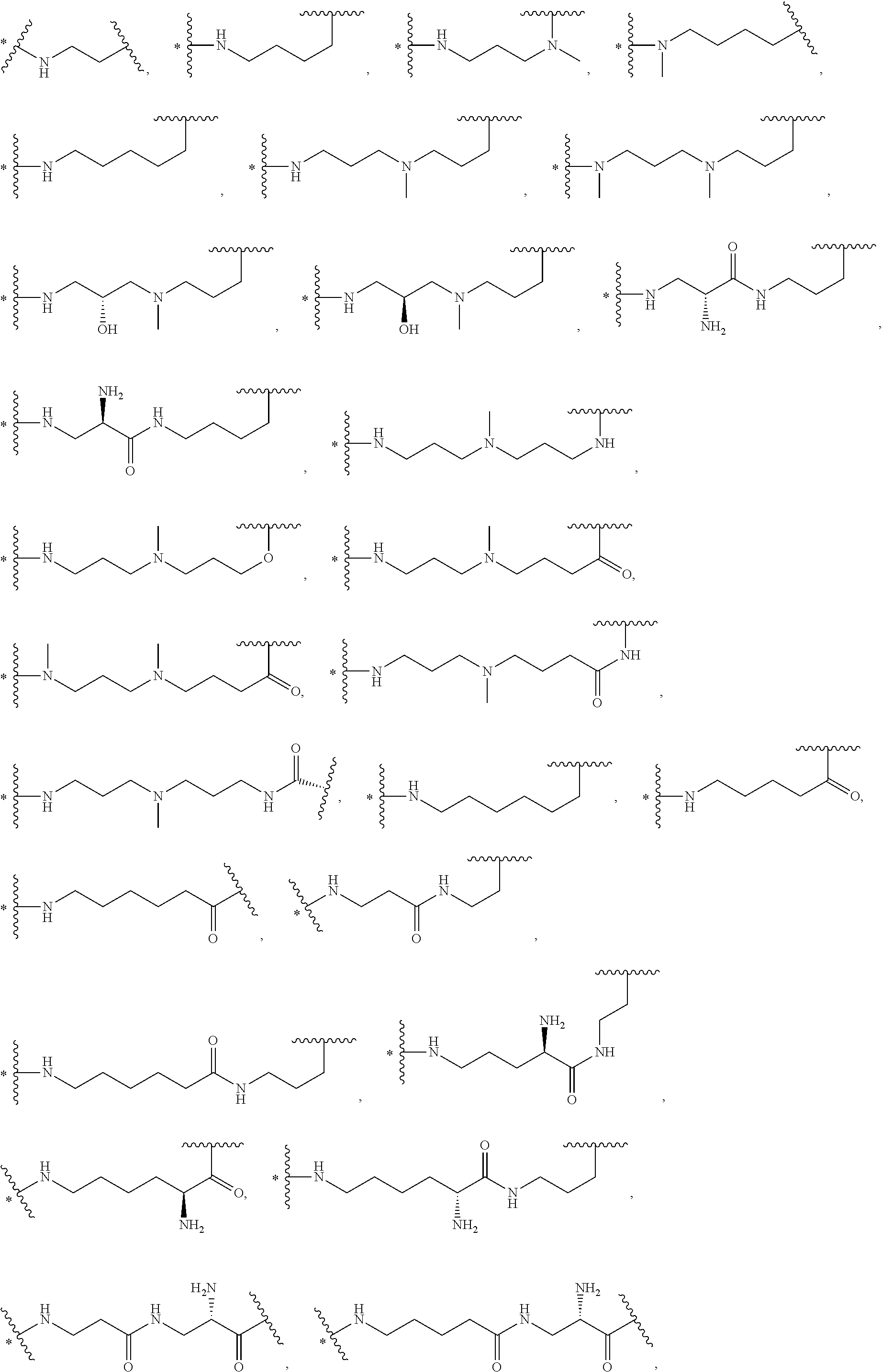

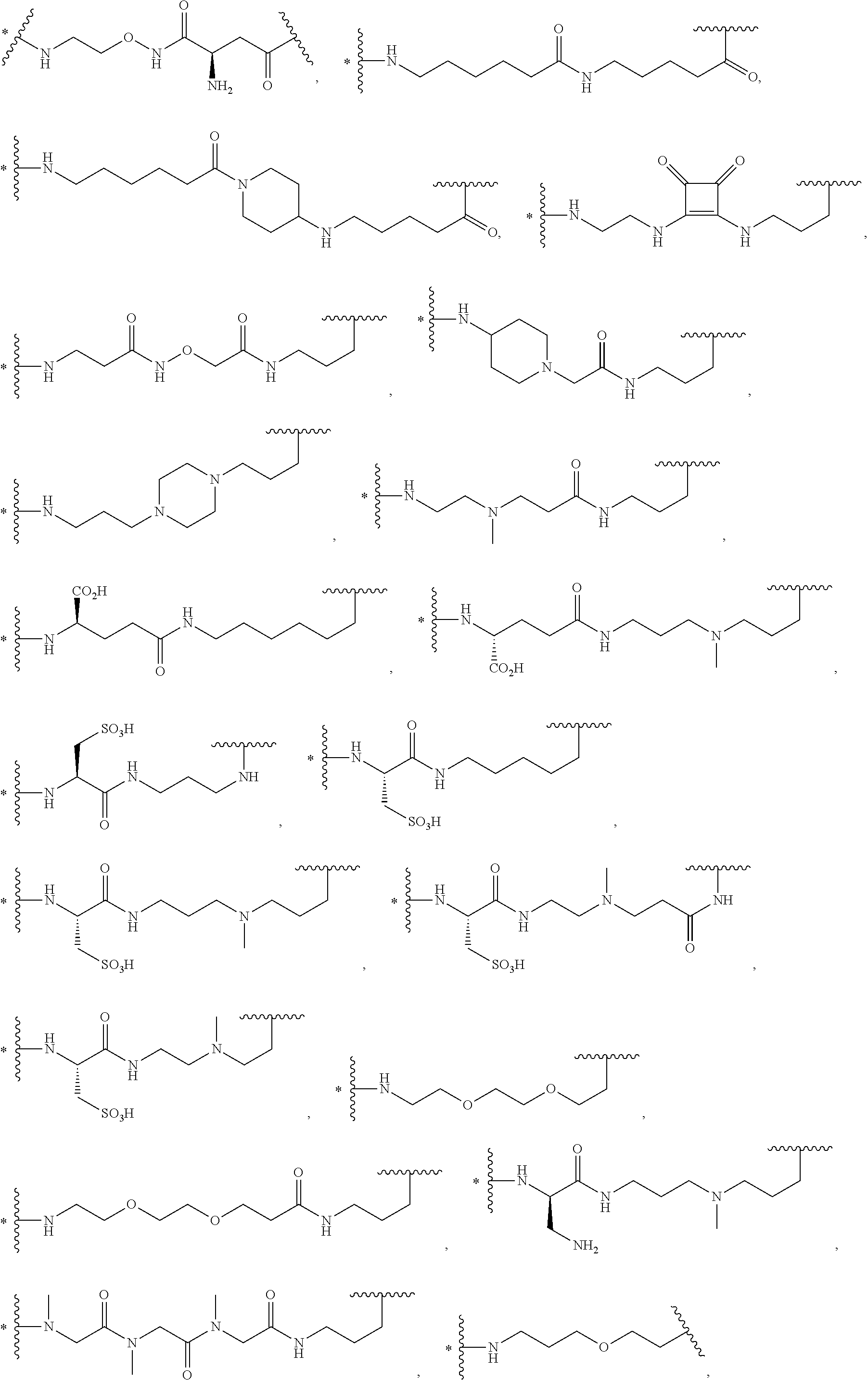

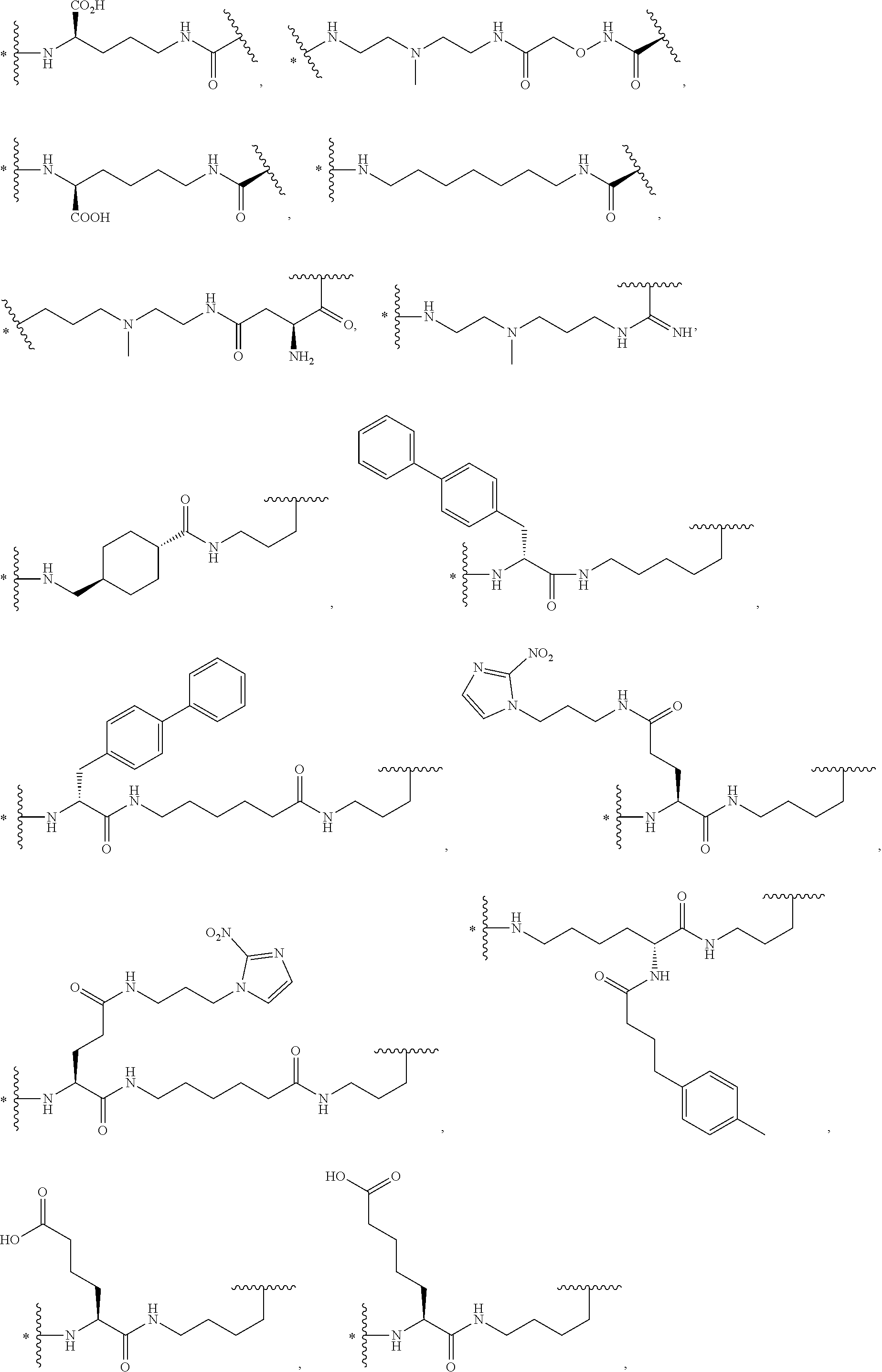

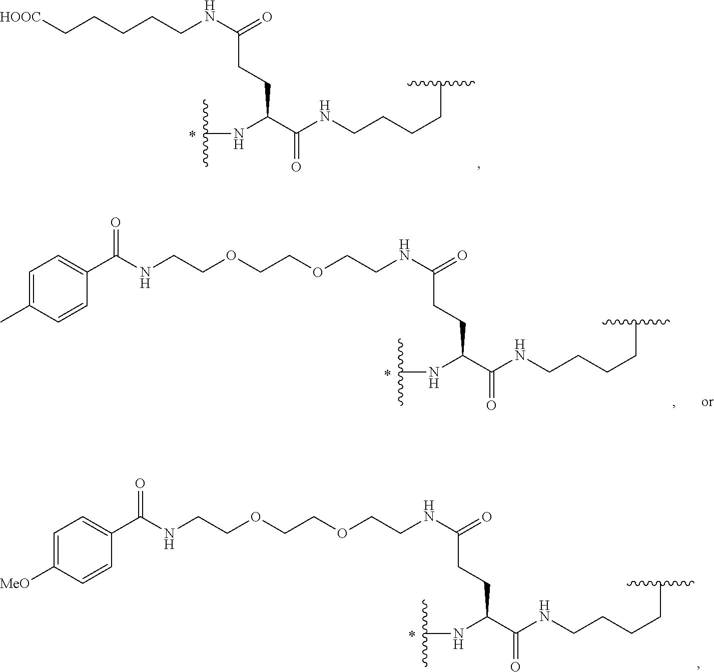 [SMILES: *CCCCNC(=O)[C@H](CCC(=O)NCCCCCC(=O)O)N* ; *CCCCNC(=O)[C@H](CCC(=O)NCCOCCOCCNC(=O)c1ccc(C)cc1)N* ; *CCCCNC(=O)[C@H](CCC(=O)NCCOCCOCCNC(=O)c1ccc(OC)cc1)N*]

wherein * denotes the attachment point of L to R^{a}.

In some embodiments, L of R^{a}-L- is:

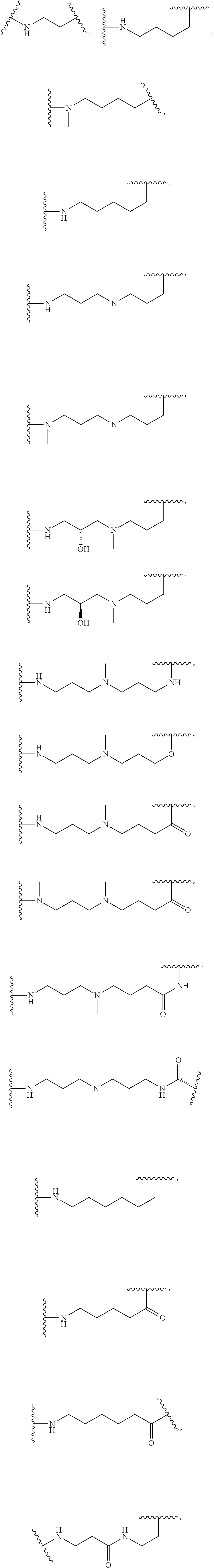

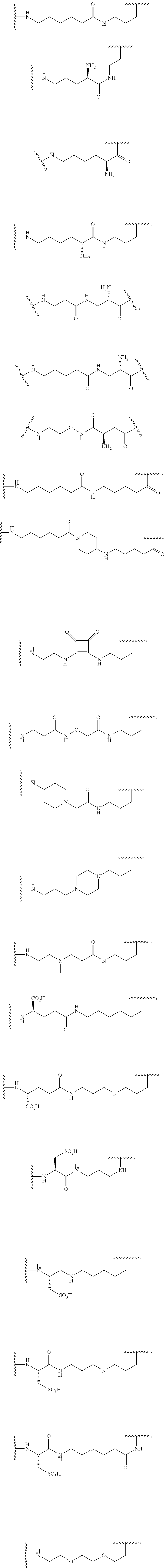

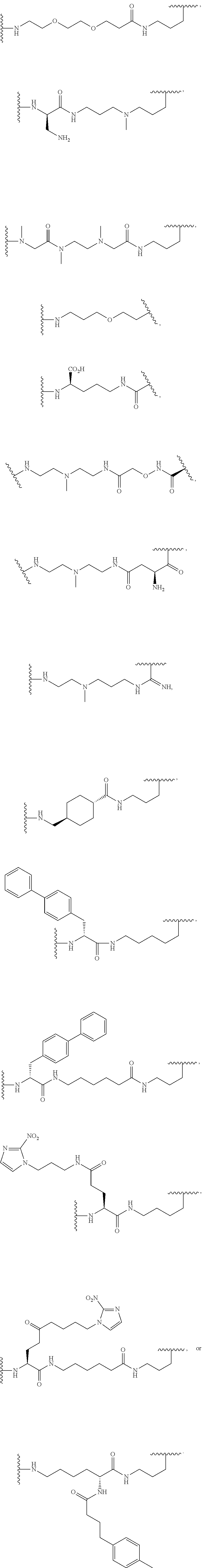

In some embodiments, L of R^{a}-L- is:

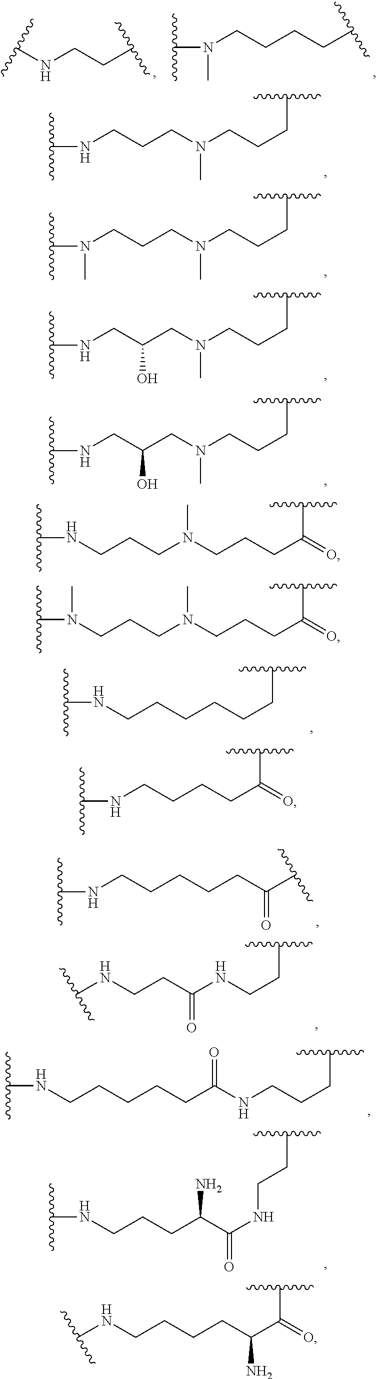

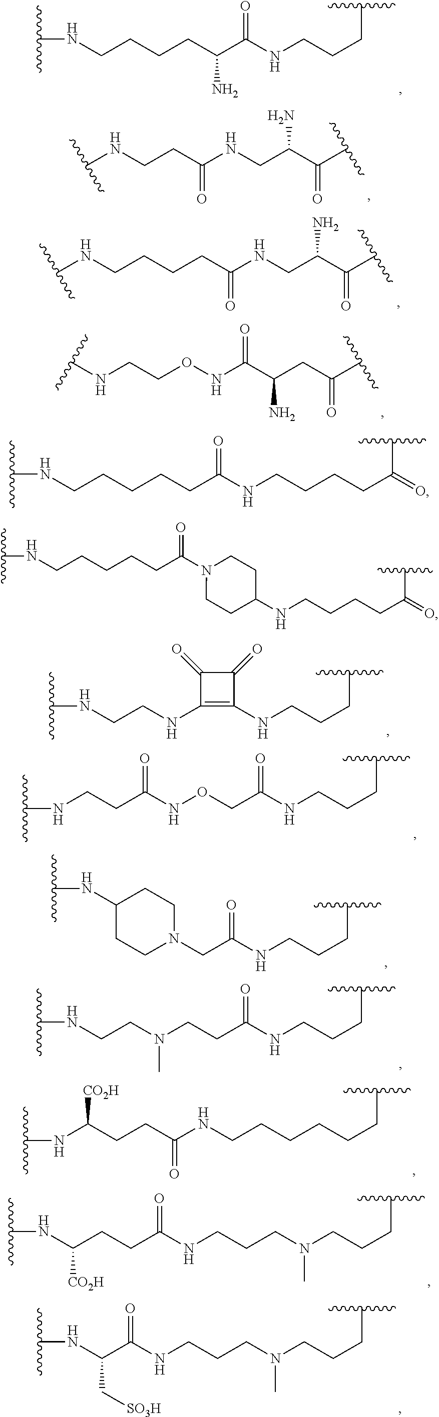

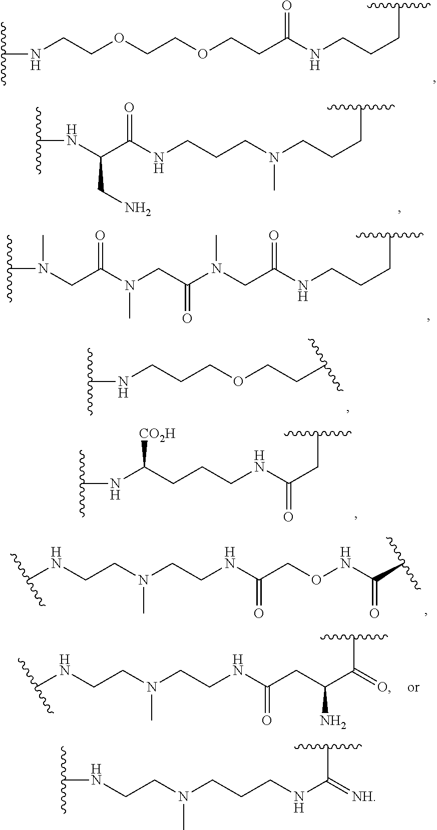

In some embodiments, L of R^{a}-L- is

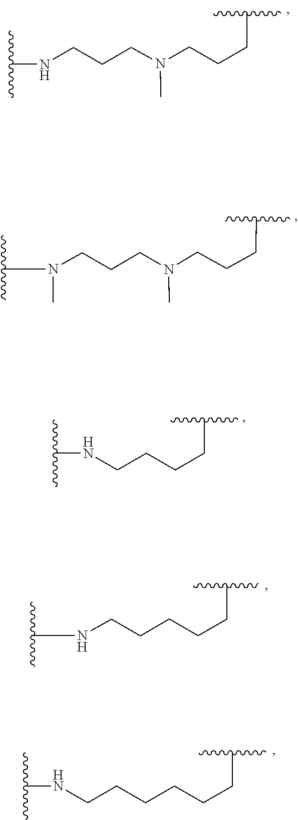 [SMILES: *CCCN(C)CCCN* ; *CCCN(C)CCCN(*)C ; *CCCCN* ; *CCCCCN* ; *CCCCCCN*]

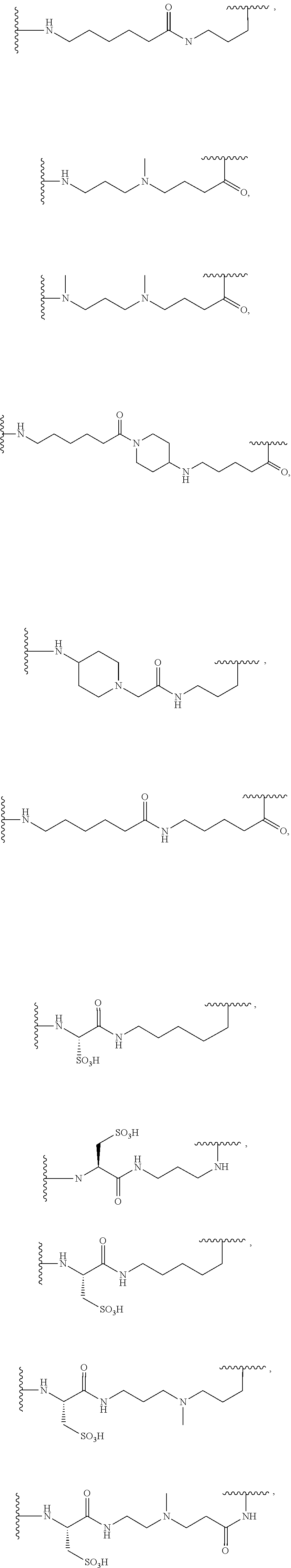

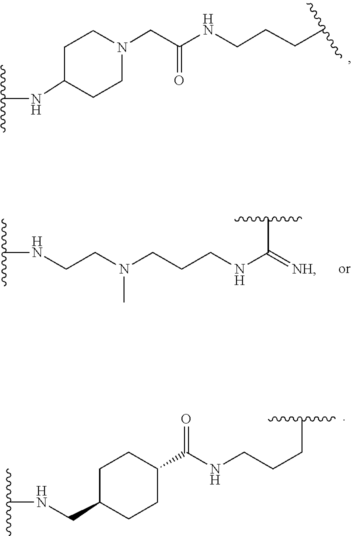 [SMILES: *CCCNC(=O)CN1CCC(N*)CC1 ; *NCCN(C)CCCNC(*)=N ; *CCCNC(=O)[C@H]1CC[C@H](CN*)CC1]

In some embodiments, L of R^{a}-L- is:

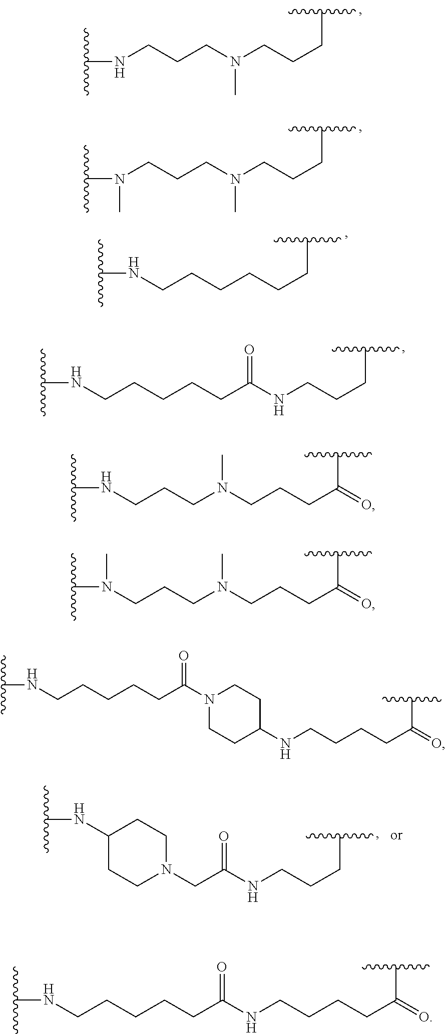

In some embodiments, L of R^{a}-L- is:

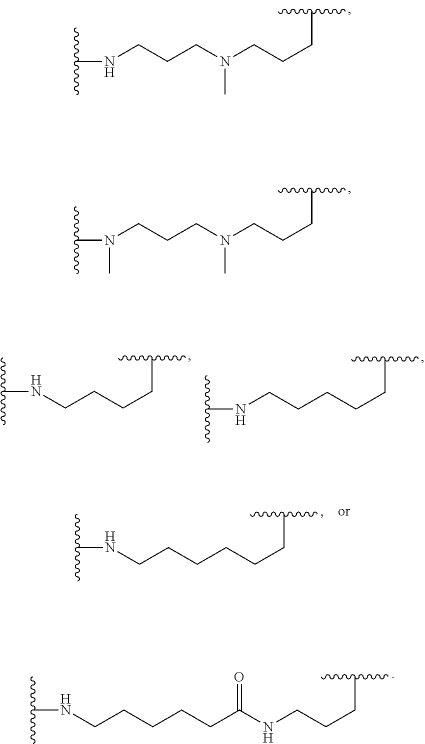 [SMILES: *CCCN(C)CCCN* ; *CCCN(C)CCCN(*)C ; *CCCCN* ; *CCCCCN* ; *CCCCCCN* ; *CCCNC(=O)CCCCCN*]

In some embodiments, L of R^{a}-L- is:

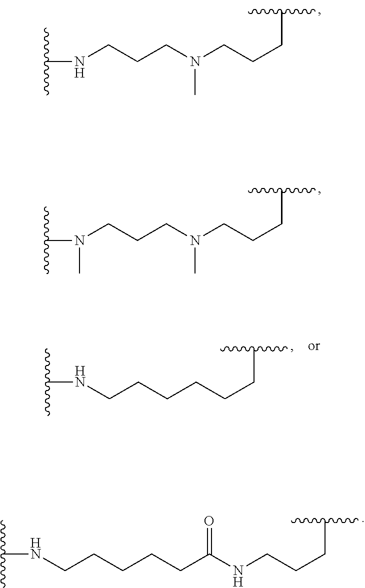 [SMILES: *CCCN(C)CCCN* ; *CCCN(C)CCCN(*)C ; *CCCCCCN* ; *CCCNC(=O)CCCCCN*]

In some embodiments, wherein L of R^{a}-L- is:

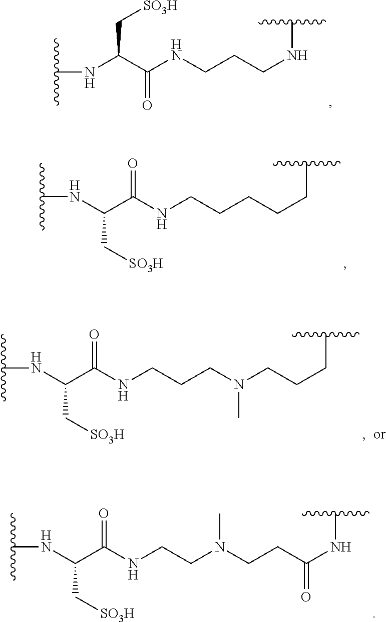 [SMILES: *NCCCNC(=O)[C@H](CS(=O)(=O)O)N* ; *CCCCCNC(=O)[C@H](CS(=O)(=O)O)N* ; *CCCN(C)CCCNC(=O)[C@H](CS(=O)(=O)O)N* ; *NC(=O)CCN(C)CCNC(=O)[C@H](CS(=O)(=O)O)N*]

In some embodiments, L of R^{a}-L- is:

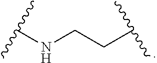 [SMILES: *CCN*]

In some embodiments, L of R^{a}-L- is:

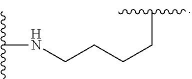 [SMILES: *CCCCN*]

In some embodiments, L of R^{a}-L- is:

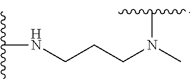 [SMILES: *NCCCN(*)C]

In some embodiments, L of R^{a}-L- is:

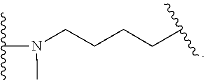 [SMILES: *CCCCN(*)C]

In some embodiments, L of R^{a}-L- is:

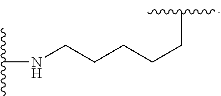 [SMILES: *CCCCCN*]

In some embodiments, L of R^{a}-L- is:

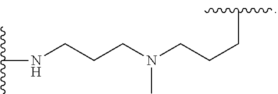 [SMILES: *CCCN(C)CCCN*]

In some embodiments, L of R-L- is:

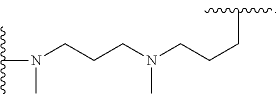 [SMILES: *CCCN(C)CCCN(*)C]

In some embodiments, L of R^{a}-L- is:

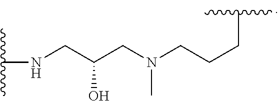 [SMILES: *CCCN(C)C[C@H](O)CN*]

In some embodiments, L of R^{a}-L- is:

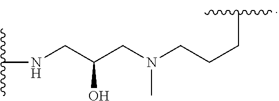 [SMILES: *CCCN(C)C[C@@H](O)CN*]

In some embodiments, L of R^{a}-L- is:

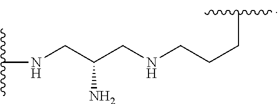 [SMILES: *CCCNC[C@H](N)CN*]

In some embodiments, L of R^{a}-L- is:

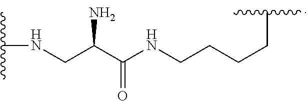 [SMILES: *CCCCNC(=O)[C@H](N)CN*]

In some embodiments, L of R^{a}-L- is:

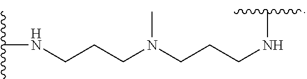 [SMILES: *NCCCN(C)CCCN*]

In some embodiments, L of R^{a}-L- is:

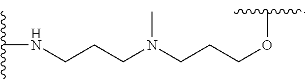 [SMILES: *NCCCN(C)CCCO*]

In some embodiments, L of R^{a}-L- is:

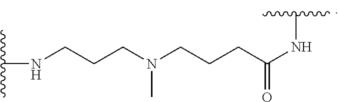 [SMILES: *NCCCN(C)CCCC(=O)N*]

In some embodiments, L of R^{a}-L- is:

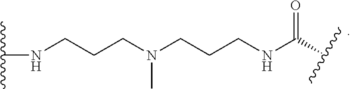

In some embodiments, L of R^{a}-L- is:

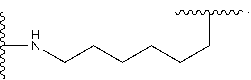 [SMILES: *CCCCCCN*]

In some embodiments, L of R^{a}-L- is:

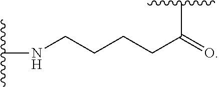 [SMILES: *NCCCCC(*)=O]

In some embodiments, L of R^{a}-L- is:

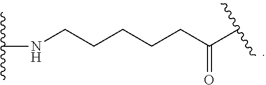 [SMILES: *NCCCCCC(*)=O]

In some embodiments, L of R^{a}-L- is:

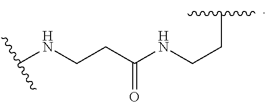 [SMILES: *CCNC(=O)CCN*]

In some embodiments, L is:

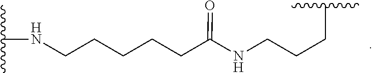 [SMILES: *CCCNC(=O)CCCCCN*]

In some embodiments, L of R^{a}-L- is:

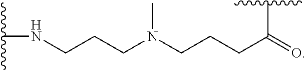 [SMILES: *NCCCN(C)CCCC(*)=O]

In some embodiments, L of R^{a}-L- is:

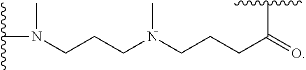 [SMILES: *C(=O)CCCN(C)CCCN(*)C]

In some embodiments, L of R^{a}-L- is:

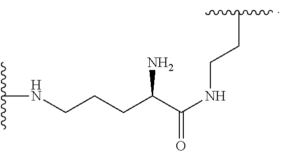 [SMILES: *CCNC(=O)[C@H](N)CCCN*]

In some embodiments, L of R^{a}-L- is:

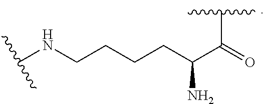 [SMILES: *NCCCC[C@H](N)C(*)=O]

In some embodiments, L of R^{a}-L- is:

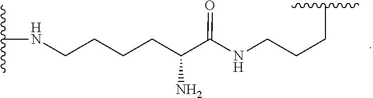 [SMILES: *CCCNC(=O)[C@H](N)CCCCN*]

In some embodiments, L of R^{a}-L- is:

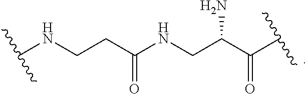 [SMILES: *NCCC(=O)NC[C@H](N)C(*)=O]

In some embodiments L is:

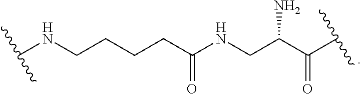 [SMILES: *NCCCCC(=O)NC[C@H](N)C(*)=O]

In some embodiments, L of R^{a}-L- is:

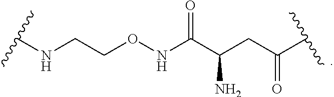 [SMILES: *NCCONC(=O)[C@H](N)CC(*)=O]

In some embodiments, L of R^{a}-L- is:

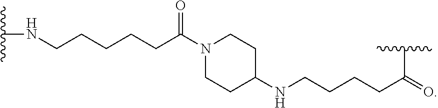 [SMILES: *NCCCCCC(=O)N1CCC(NCCCCC(*)=O)CC1]

In some embodiments, L of R^{a}-L- is:

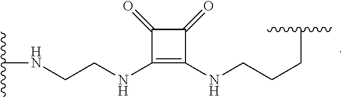 [SMILES: *CCCNc1c(NCCN*)c(=O)c1=O]

In some embodiments, L of R^{a}-L- is:

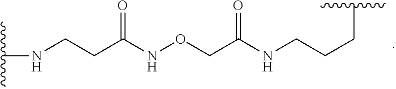 [SMILES: *CCCNC(=O)CONC(=O)CCN*]

H n some embodiments, L of R^{a}-L- is:

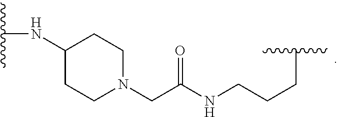 [SMILES: *CCCNC(=O)CN1CCC(N*)CC1]

In some embodiments, L is:

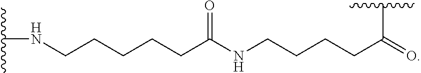 [SMILES: *NCCCCCC(=O)NCCCCC(*)=O]

In some embodiments, L of R^{a}-L- is:

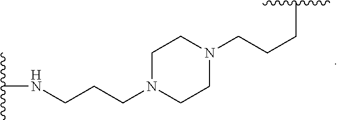 [SMILES: *CCCN1CCN(CCCN*)CC1]

In some embodiments, L of R^{a}-L- is:

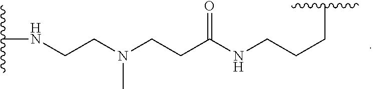 [SMILES: *CCCNC(=O)CCN(C)CCN*]

In some embodiments, L of R^{a}-L- is:

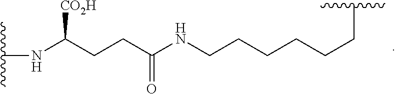 [SMILES: *CCCCCCNC(=O)CC[C@@H](N*)C(=O)O]

In some embodiments, L of R^{a}-L- is:

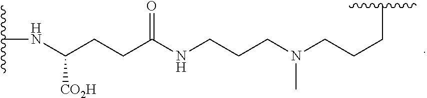 [SMILES: *CCCN(C)CCCNC(=O)CC[C@@H](N*)C(=O)O]

In some embodiments, L of R^{a}-L- is:

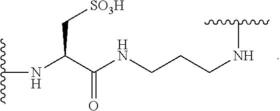 [SMILES: *NCCCNC(=O)[C@H](CS(=O)(=O)O)N*]

In some embodiments, L of R^{a}-L- is:

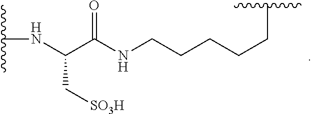 [SMILES: *CCCCCNC(=O)[C@H](CS(=O)(=O)O)N*]

In some embodiments, L of R^{a}-L- is:

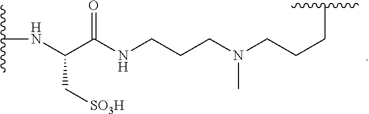 [SMILES: *CCCN(C)CCCNC(=O)[C@H](CS(=O)(=O)O)N*]

In some embodiments, L of R^{a}-L- is:

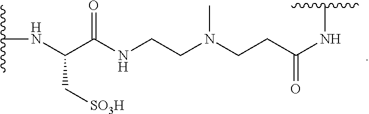 [SMILES: *NC(=O)CCN(C)CCNC(=O)[C@H](CS(=O)(=O)O)N*]

In some embodiments, L of R^{a}-L- is:

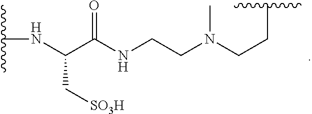 [SMILES: *CCN(C)CCNC(=O)[C@H](CS(=O)(=O)O)N*]

In some embodiments, L of R^{a}-L- is:

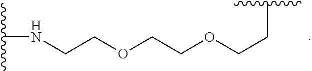 [SMILES: *CCOCCOCCN*]

In some embodiments, L of R^{a}-L- is:

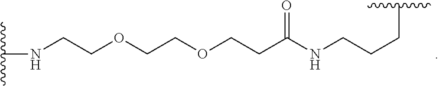 [SMILES: *CCCNC(=O)CCOCCOCCN*]

In some embodiments, L of R^{a}-L- is:

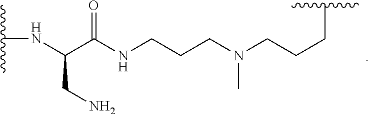 [SMILES: *CCCN(C)CCCNC(=O)[C@@H](CN)N*]

In some embodiments, L of R^{a}-L- is:

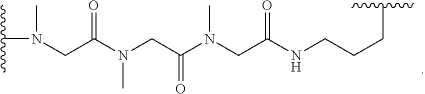 [SMILES: *CCCNC(=O)CN(C)C(=O)CN(C)C(=O)CN(*)C]

In some embodiments, L of R^{a}-L- is:

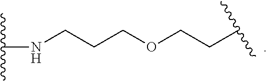 [SMILES: *CCOCCCN*]

In some embodiments, L of R^{a}-L- is:

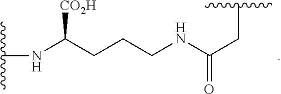 [SMILES: *CC(=O)NCCC[C@@H](N*)C(=O)O]

In some embodiments, L of R^{a}-L- is:

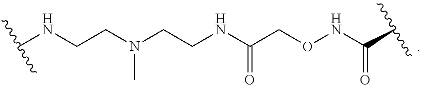

In some embodiments, L of R^{a}-L- is:

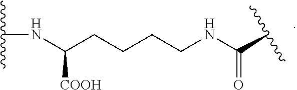

In some embodiments, L of R^{a}-L- is:

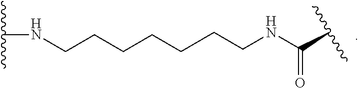

In some embodiments, L of R^{a}-L- is:

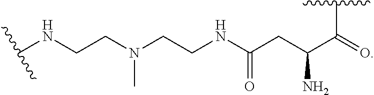 [SMILES: *NCCN(C)CCNC(=O)C[C@H](N)C(*)=O]

In some embodiments, L of R^{a}-L- is:

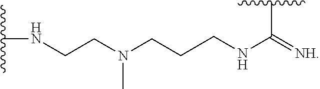 [SMILES: *NCCN(C)CCCNC(*)=N]

In some embodiments, L of R^{a}-L- is:

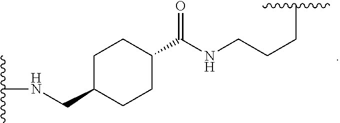 [SMILES: *CCCNC(=O)[C@H]1CC[C@H](CN*)CC1]

some embodiments, L of R^{a}-L- is:

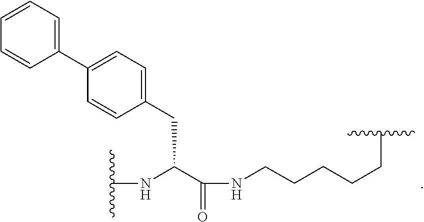 [SMILES: *CCCCCNC(=O)[C@@H](Cc1ccc(-c2ccccc2)cc1)N*]

In some embodiments, L of R^{a}-L- is:

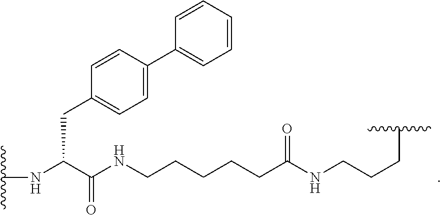 [SMILES: *CCCNC(=O)CCCCCNC(=O)[C@@H](Cc1ccc(-c2ccccc2)cc1)N*]

In some embodiments, L of R^{a}-L- is:

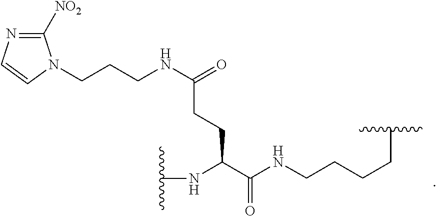 [SMILES: *CCCCNC(=O)[C@H](CCC(=O)NCCCn1ccnc1[N+](=O)[O-])N*]

In some embodiments, L of R^{a}-L- is:

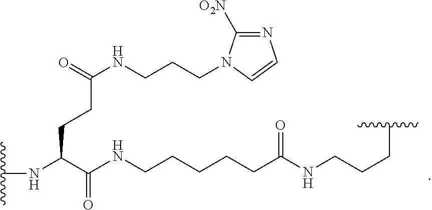 [SMILES: *CCCNC(=O)CCCCCNC(=O)[C@H](CCC(=O)NCCCn1ccnc1[N+](=O)[O-])N*]

In some embodiments, L of R^{a}-L- is:

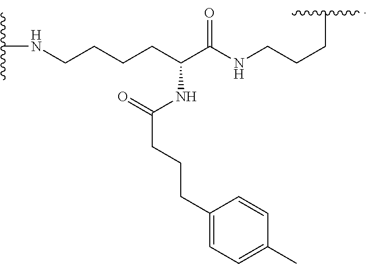 [SMILES: *CCCNC(=O)[C@@H](CCCCN*)NC(=O)CCCc1ccc(C)cc1]

In some embodiments, L of R^{a}-L- is:

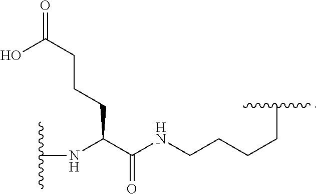 [SMILES: *CCCCNC(=O)[C@H](CCCC(=O)O)N*]

In some embodiments, L of R^{a}-L- is:

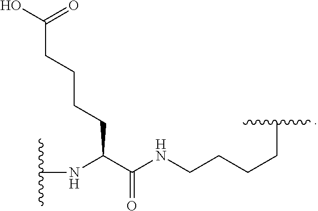 [SMILES: *CCCCNC(=O)[C@H](CCCCC(=O)O)N*]

In some embodiments, L of R^{a}-L- is:

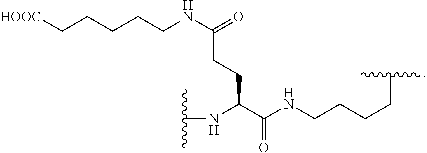 [SMILES: *CCCCNC(=O)[C@H](CCC(=O)NCCCCCC(=O)O)N*]

In some embodiments, L of R^{a}-L- is:

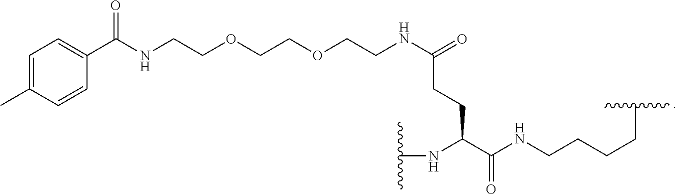 [SMILES: *CCCCNC(=O)[C@H](CCC(=O)NCCOCCOCCNC(=O)c1ccc(C)cc1)N*]

In some embodiments, L of R^{a}-L- is:

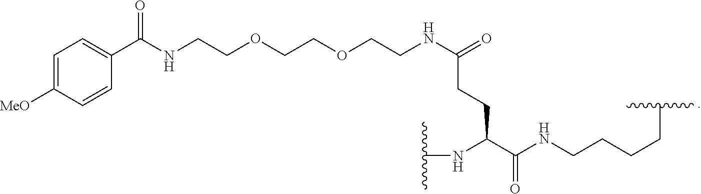 [SMILES: *CCCCNC(=O)[C@H](CCC(=O)NCCOCCOCCNC(=O)c1ccc(OC)cc1)N*]

### Representative Linker and Chelating Moieties

In some embodiments, R^{a}-L- is:

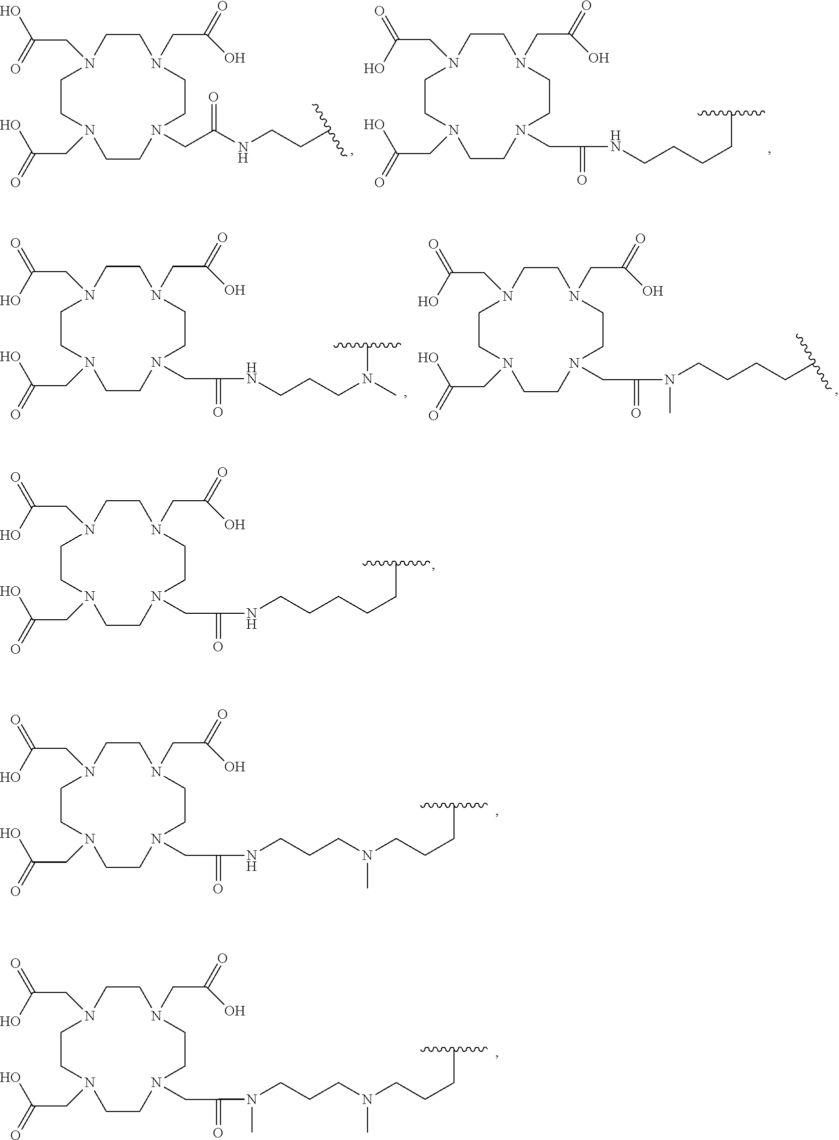

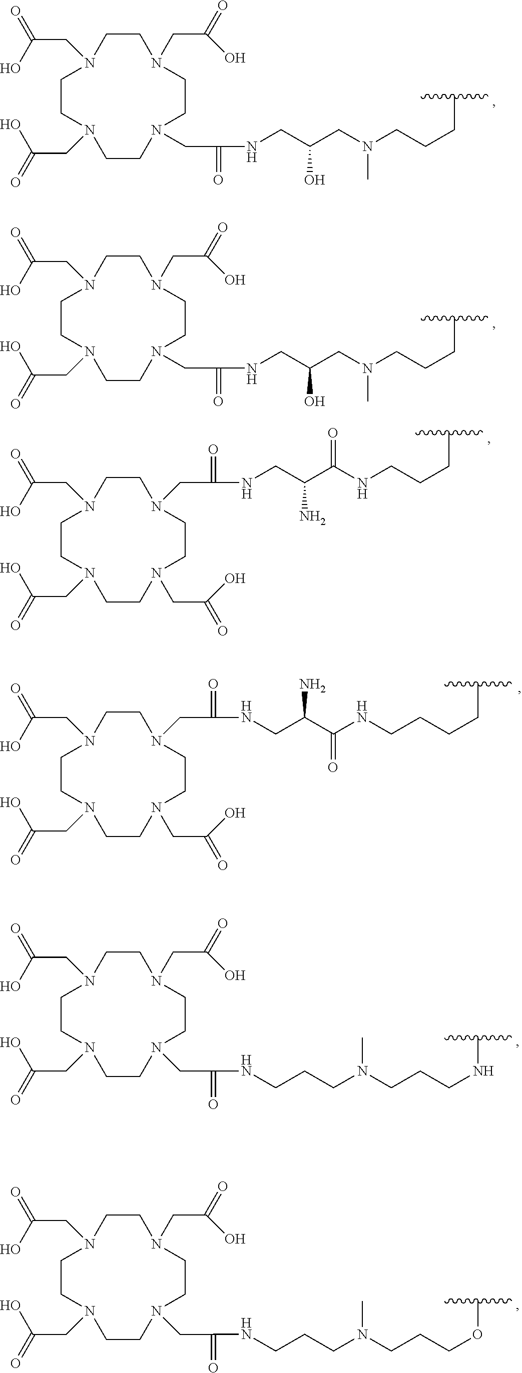

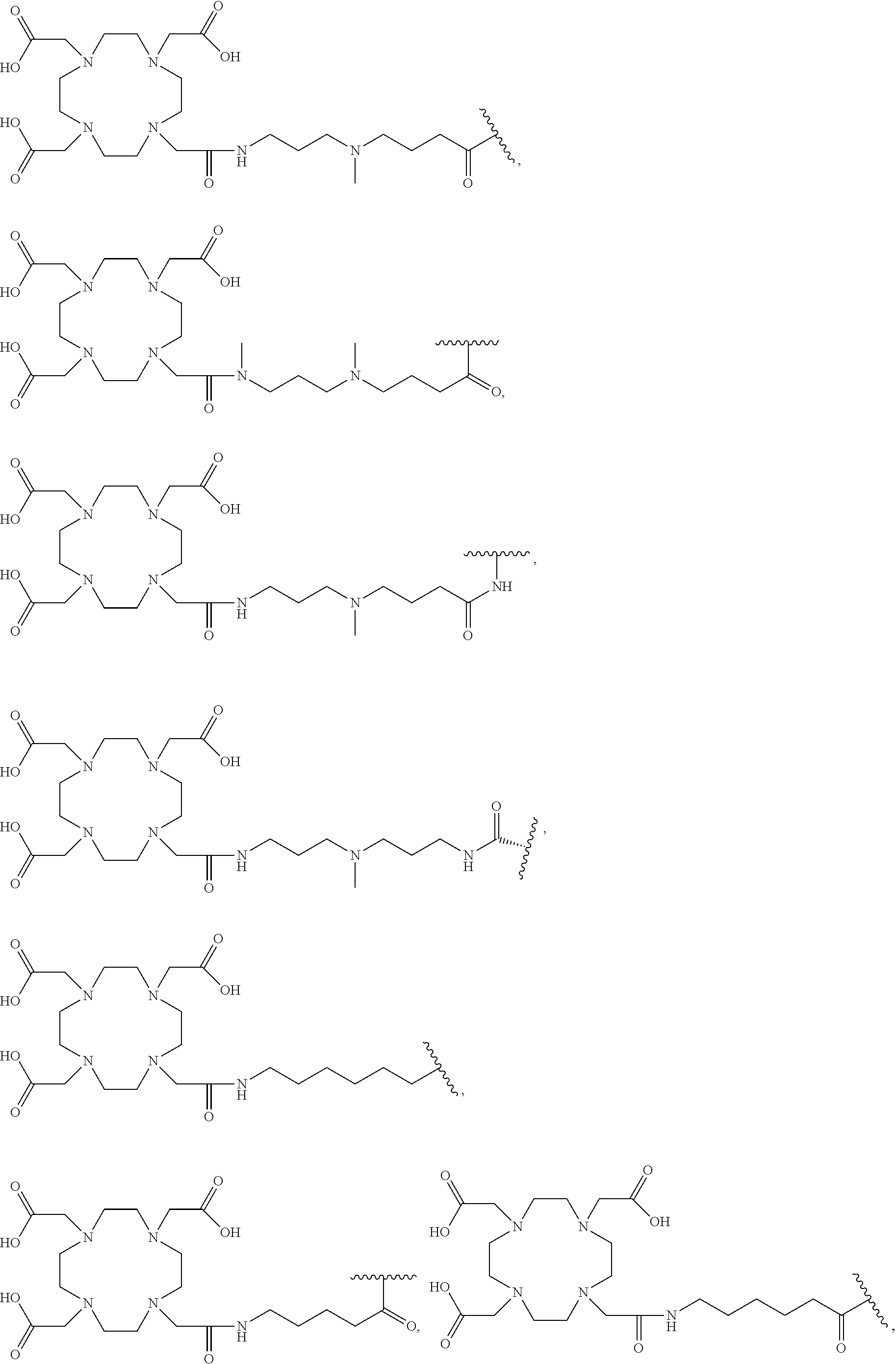

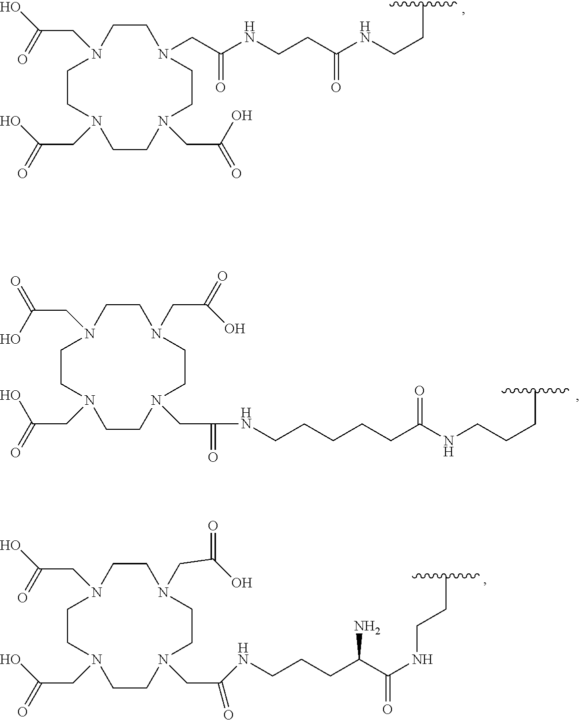 [SMILES: *CCNC(=O)CCNC(=O)CN1CCN(CC(=O)O)CCN(CC(=O)O)CCN(CC(=O)O)CC1 ; *CCCNC(=O)CCCCCNC(=O)CN1CCN(CC(=O)O)CCN(CC(=O)O)CCN(CC(=O)O)CC1 ; *CCNC(=O)[C@H](N)CCCNC(=O)CN1CCN(CC(=O)O)CCN(CC(=O)O)CCN(CC(=O)O)CC1]

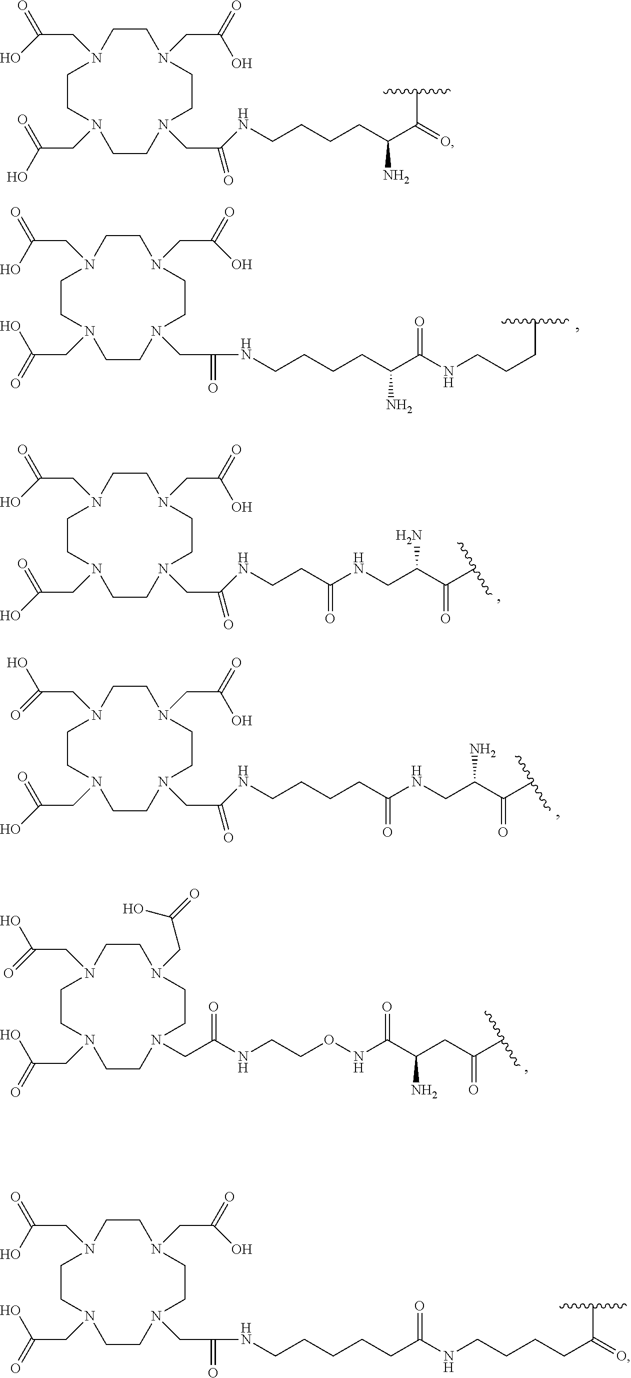

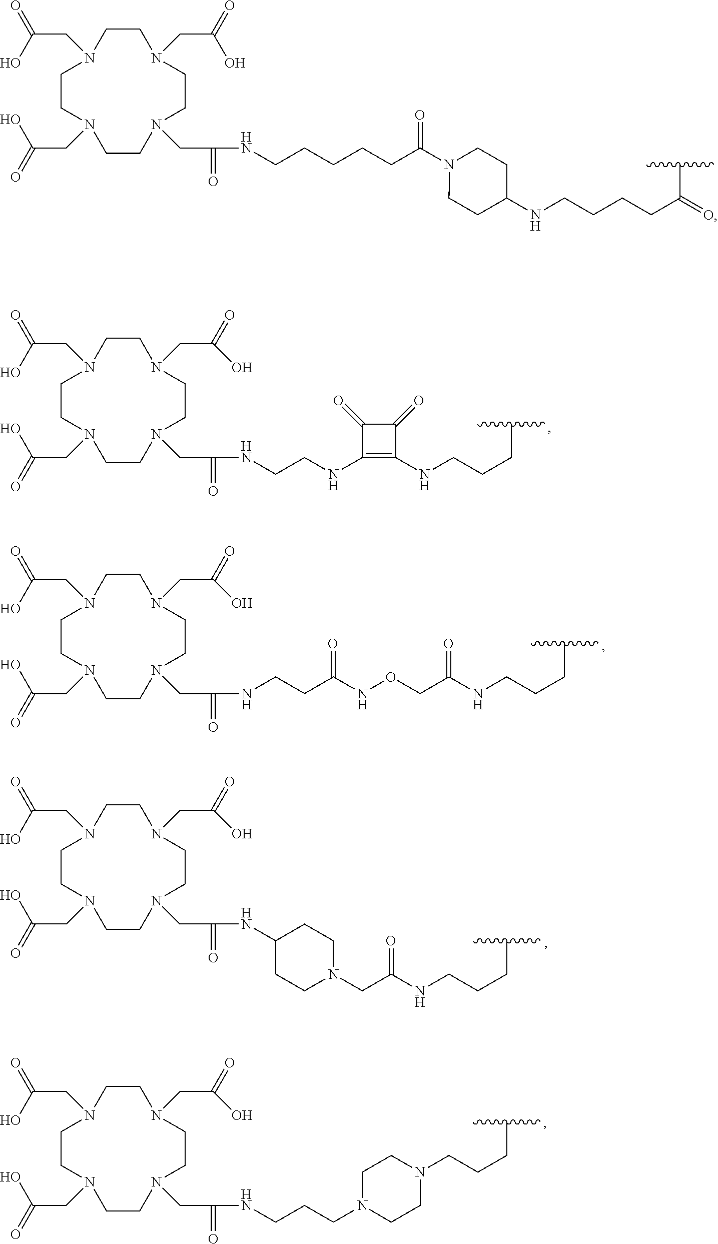 [SMILES: *C(=O)CCCCNC1CCN(C(=O)CCCCCNC(=O)CN2CCN(CC(=O)O)CCN(CC(=O)O)CCN(CC(=O)O)CC2)CC1 ; *CCCNc1c(NCCNC(=O)CN2CCN(CC(=O)O)CCN(CC(=O)O)CCN(CC(=O)O)CC2)c(=O)c1=O ; *CCCNC(=O)CONC(=O)CCNC(=O)CN1CCN(CC(=O)O)CCN(CC(=O)O)CCN(CC(=O)O)CC1 ; *CCCNC(=O)CN1CCC(NC(=O)CN2CCN(CC(=O)O)CCN(CC(=O)O)CCN(CC(=O)O)CC2)CC1 ; *CCCN1CCN(CCCNC(=O)CN2CCN(CC(=O)O)CCN(CC(=O)O)CCN(CC(=O)O)CC2)CC1]

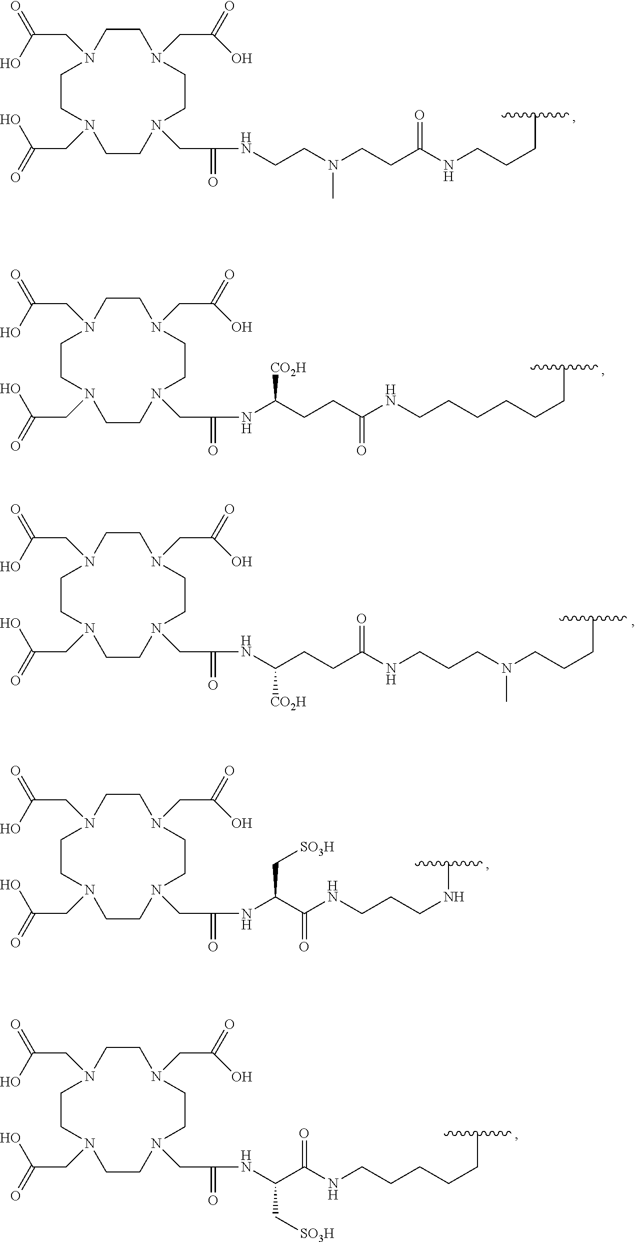 [SMILES: *CCCNC(=O)CCN(C)CCNC(=O)CN1CCN(CC(=O)O)CCN(CC(=O)O)CCN(CC(=O)O)CC1 ; *CCCCCCNC(=O)CC[C@@H](NC(=O)CN1CCN(CC(=O)O)CCN(CC(=O)O)CCN(CC(=O)O)CC1)C(=O)O ; *CCCN(C)CCCNC(=O)CC[C@@H](NC(=O)CN1CCN(CC(=O)O)CCN(CC(=O)O)CCN(CC(=O)O)CC1)C(=O)O ; *NCCCNC(=O)[C@H](CS(=O)(=O)O)NC(=O)CN1CCN(CC(=O)O)CCN(CC(=O)O)CCN(CC(=O)O)CC1 ; *CCCCCNC(=O)[C@H](CS(=O)(=O)O)NC(=O)CN1CCN(CC(=O)O)CCN(CC(=O)O)CCN(CC(=O)O)CC1]

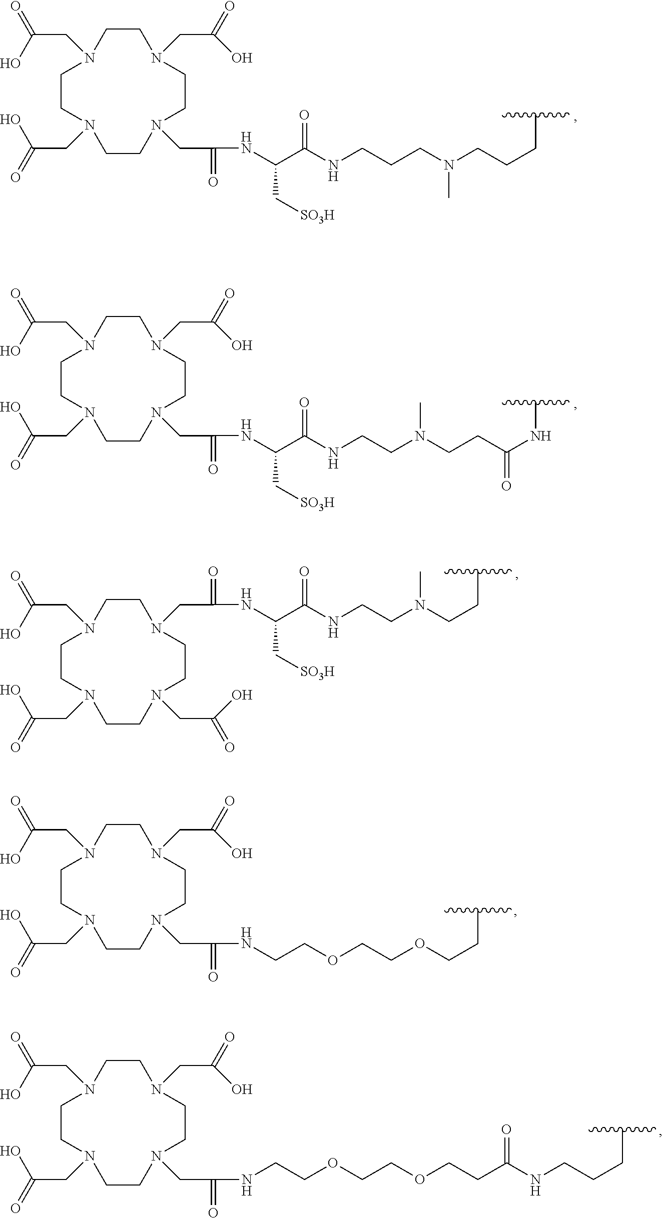 [SMILES: *CCCN(C)CCCNC(=O)[C@H](CS(=O)(=O)O)NC(=O)CN1CCN(CC(=O)O)CCN(CC(=O)O)CCN(CC(=O)O)CC1 ; *NC(=O)CCN(C)CCNC(=O)[C@H](CS(=O)(=O)O)NC(=O)CN1CCN(CC(=O)O)CCN(CC(=O)O)CCN(CC(=O)O)CC1 ; *CCN(C)CCNC(=O)[C@H](CS(=O)(=O)O)NC(=O)CN1CCN(CC(=O)O)CCN(CC(=O)O)CCN(CC(=O)O)CC1 ; *CCOCCOCCNC(=O)CN1CCN(CC(=O)O)CCN(CC(=O)O)CCN(CC(=O)O)CC1 ; *CCCNC(=O)CCOCCOCCNC(=O)CN1CCN(CC(=O)O)CCN(CC(=O)O)CCN(CC(=O)O)CC1]

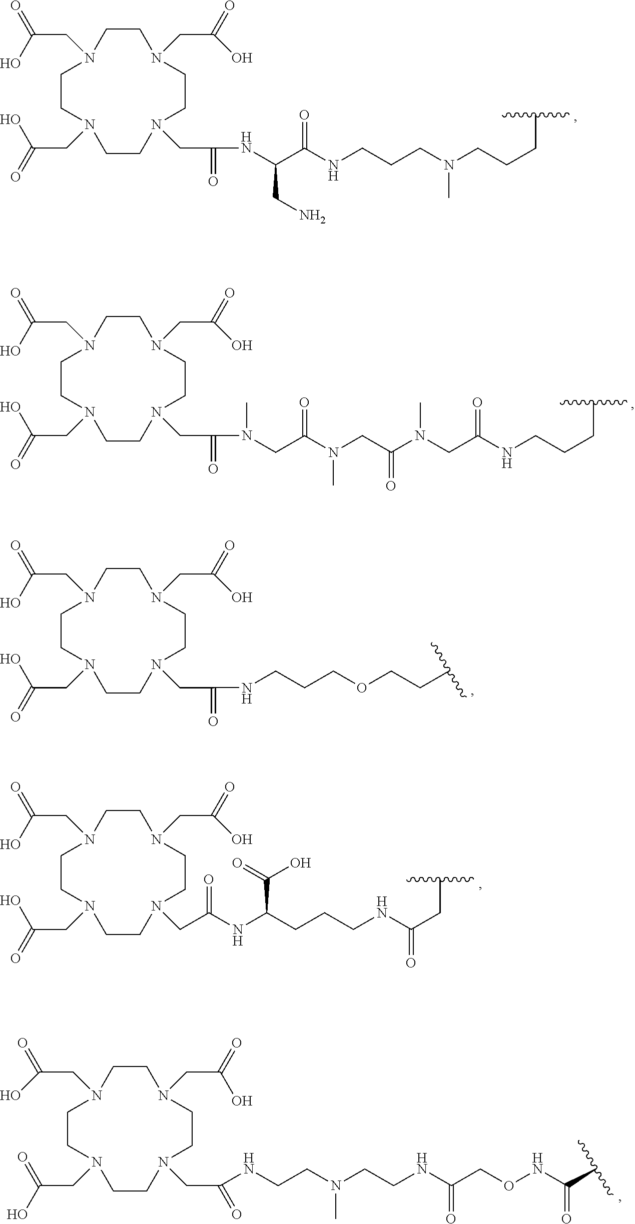

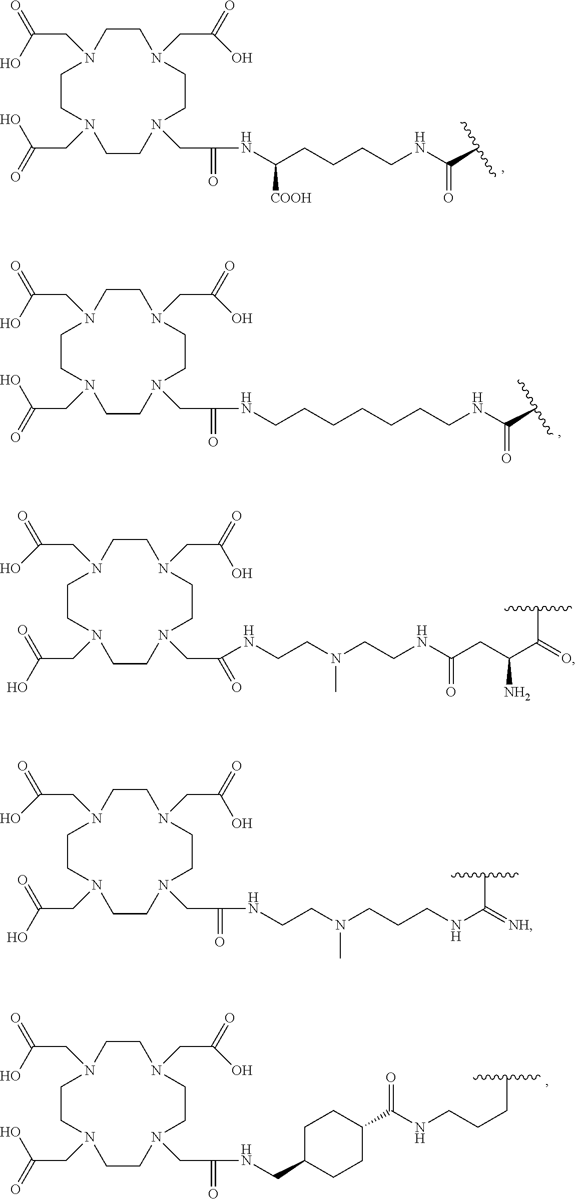

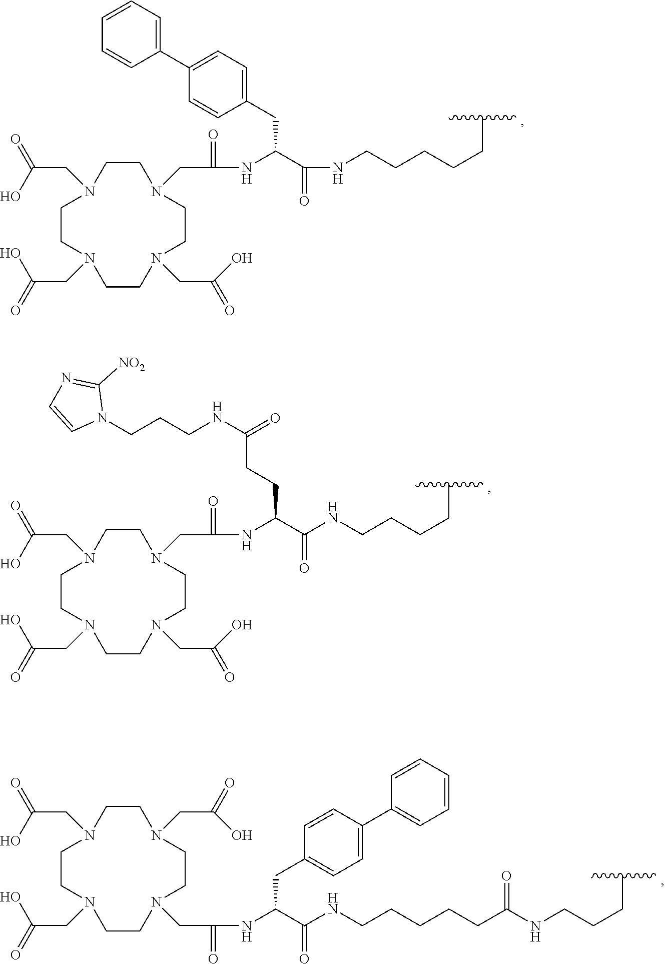 [SMILES: *CCCCCNC(=O)[C@@H](Cc1ccc(-c2ccccc2)cc1)NC(=O)CN1CCN(CC(=O)O)CCN(CC(=O)O)CCN(CC(=O)O)CC1 ; *CCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)CCCCCNC(=O)[C@@H](Cc1ccc(-c2ccccc2)cc1)NC(=O)CN1CCN(CC(=O)O)CCN(CC(=O)O)CCN(CC(=O)O)CC1]

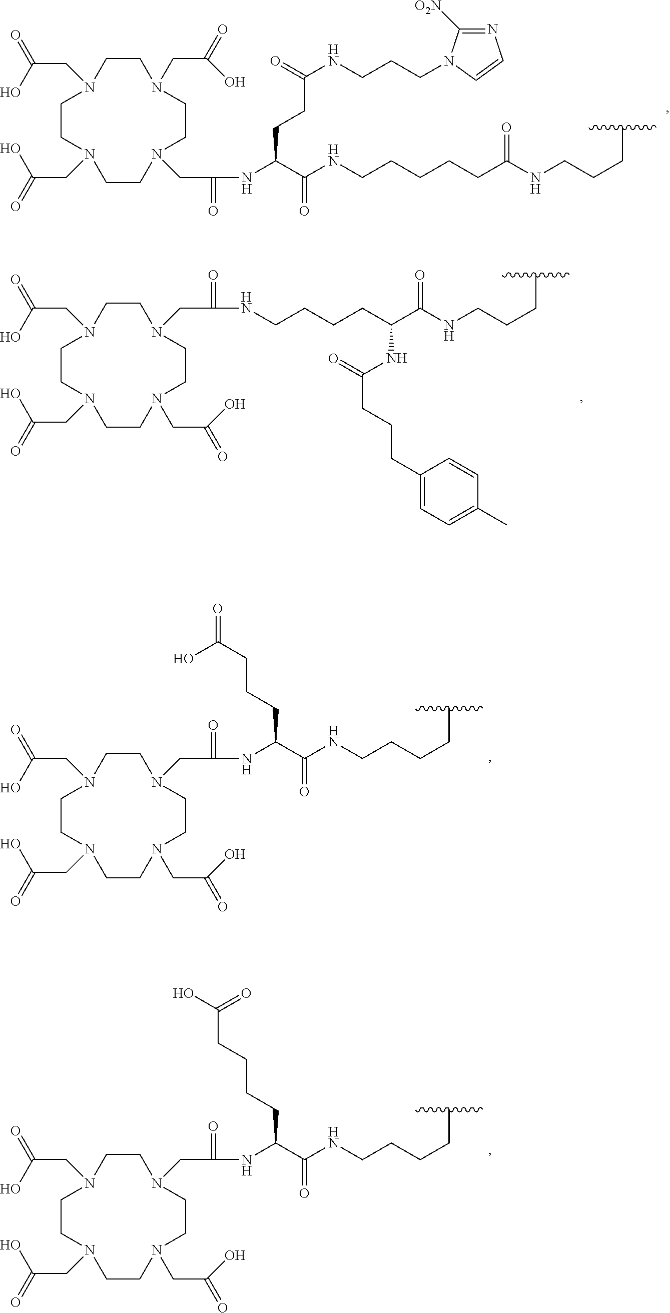 [SMILES: *CCCNC(=O)CCCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)[C@@H](CCCCNC(=O)CN1CCN(CC(=O)O)CCN(CC(=O)O)CCN(CC(=O)O)CC1)NC(=O)CCCc1ccc(C)cc1 ; *CCCCNC(=O)[C@H](CCCC(=O)O)NC(=O)CN1CCN(CC(=O)O)CCN(CC(=O)O)CCN(CC(=O)O)CC1 ; *CCCCNC(=O)[C@H](CCCCC(=O)O)NC(=O)CN1CCN(CC(=O)O)CCN(CC(=O)O)CCN(CC(=O)O)CC1]

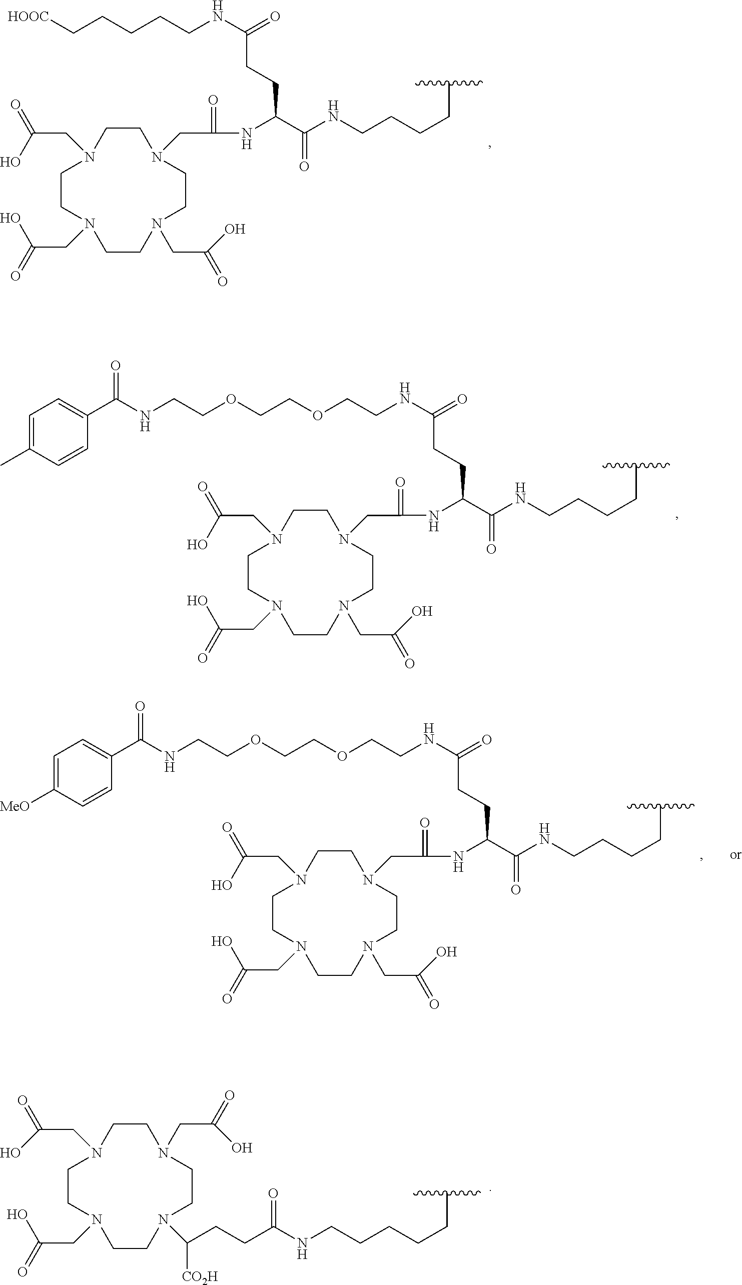 [SMILES: *CCCCNC(=O)[C@H](CCC(=O)NCCCCCC(=O)O)NC(=O)CN1CCN(CC(=O)O)CCN(CC(=O)O)CCN(CC(=O)O)CC1 ; *CCCCNC(=O)[C@H](CCC(=O)NCCOCCOCCNC(=O)c1ccc(C)cc1)NC(=O)CN1CCN(CC(=O)O)CCN(CC(=O)O)CCN(CC(=O)O)CC1 ; *CCCCNC(=O)[C@H](CCC(=O)NCCOCCOCCNC(=O)c1ccc(OC)cc1)NC(=O)CN1CCN(CC(=O)O)CCN(CC(=O)O)CCN(CC(=O)O)CC1 ; *CCCCCNC(=O)CCC(C(=O)O)N1CCN(CC(=O)O)CCN(CC(=O)O)CCN(CC(=O)O)CC1]

In some embodiments, R^{a}-L- is

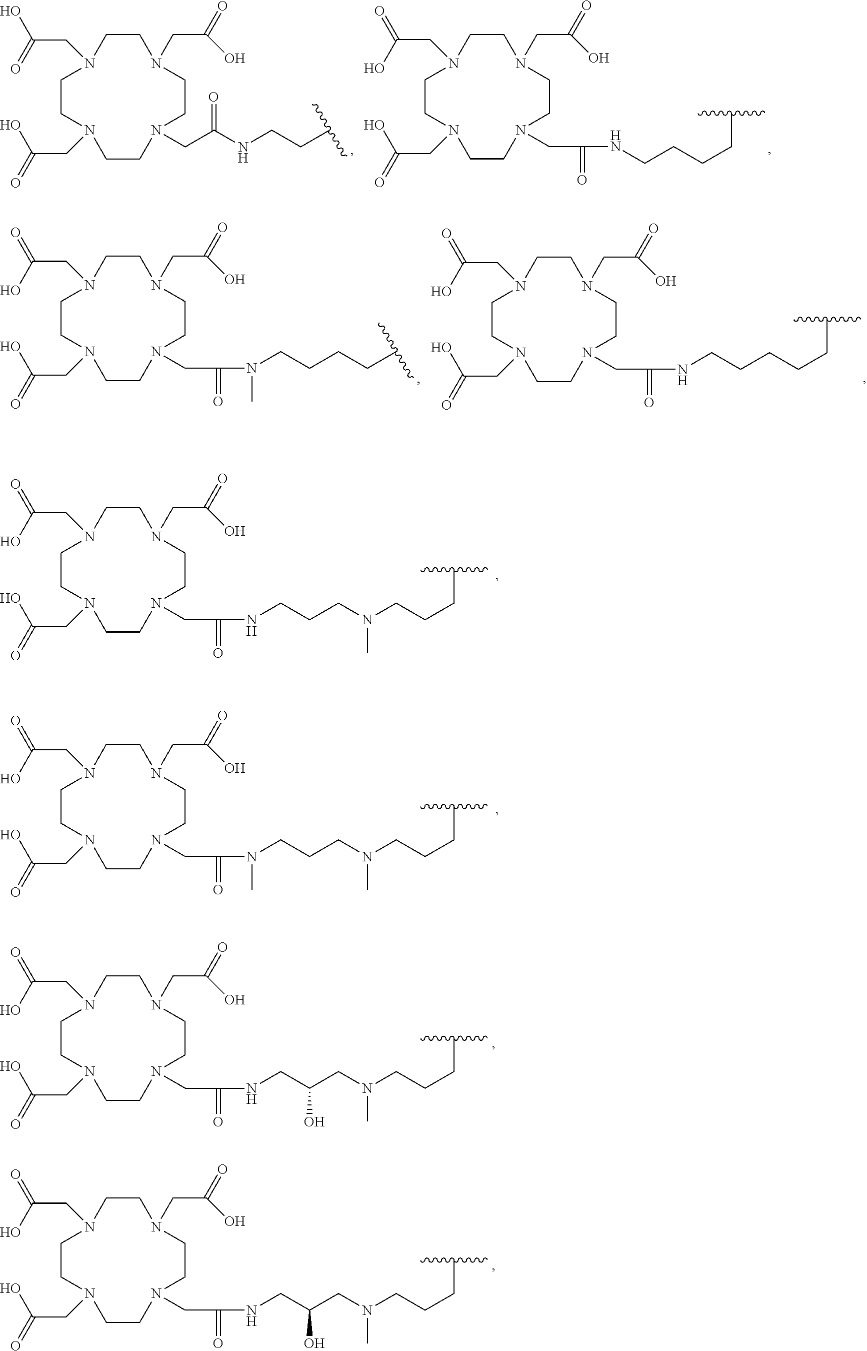

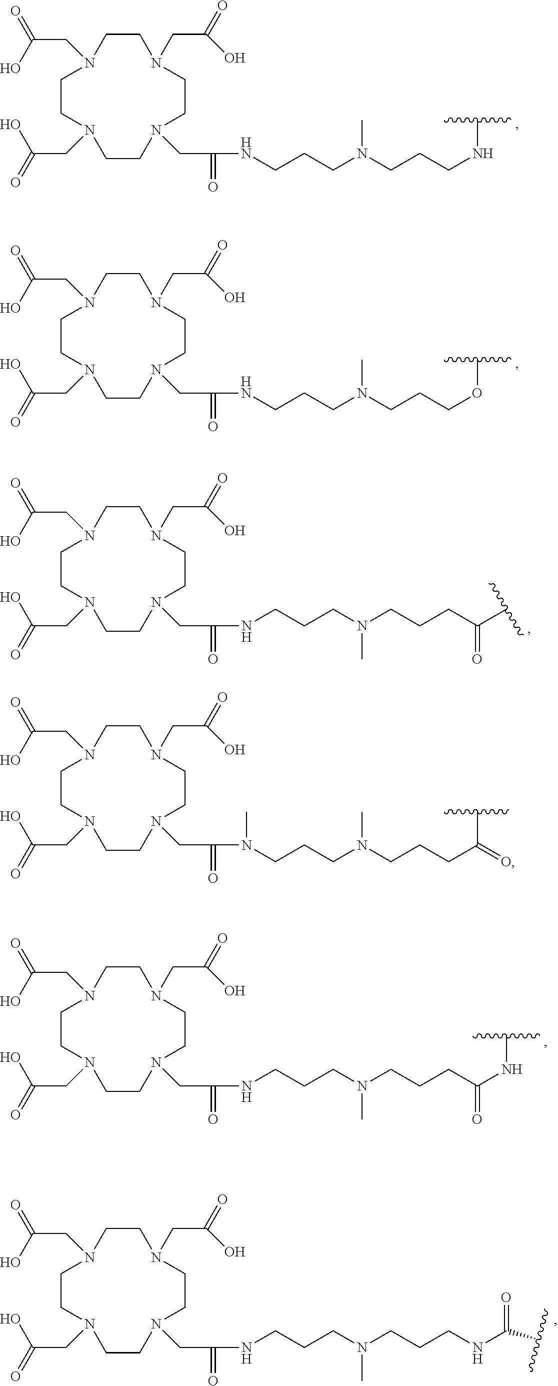

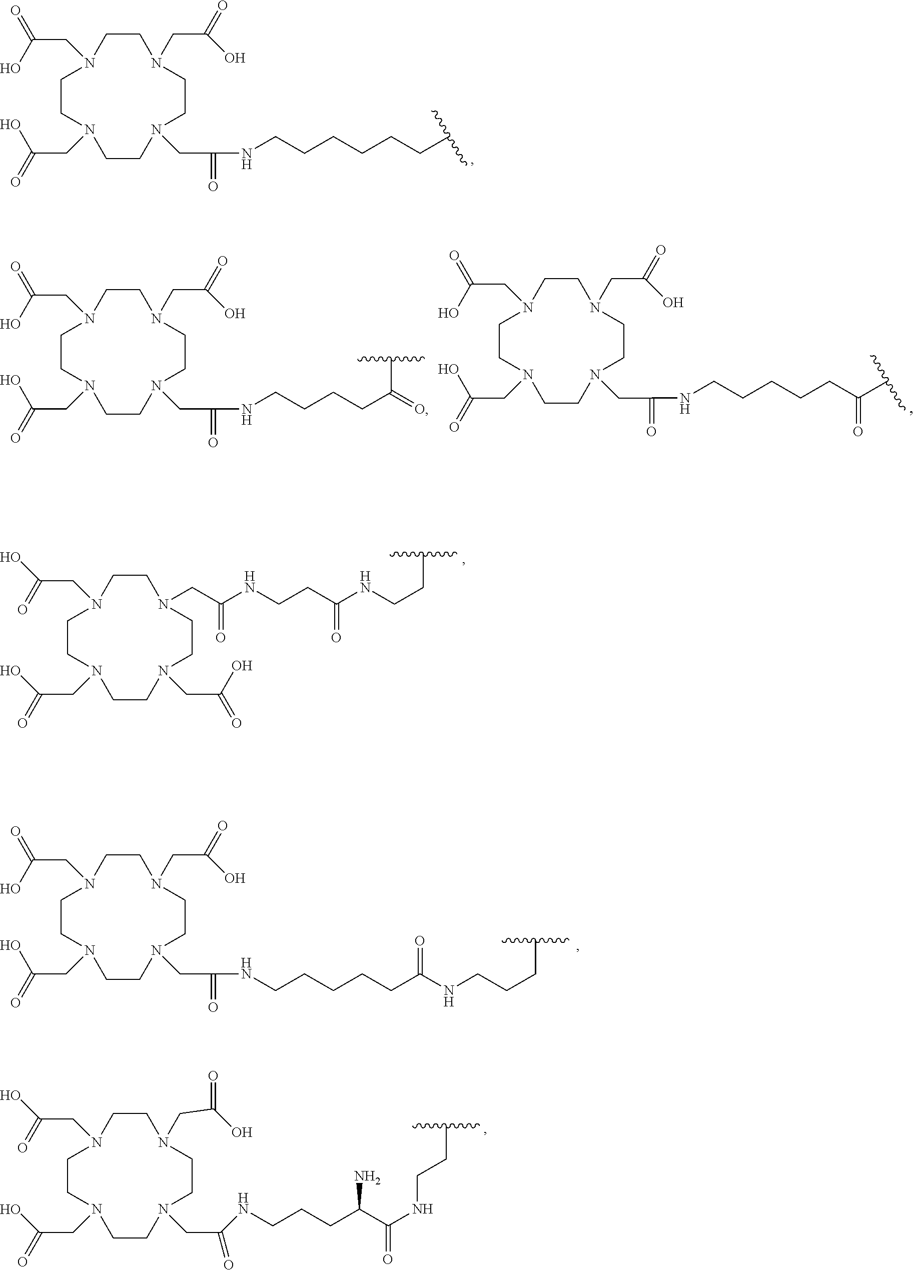

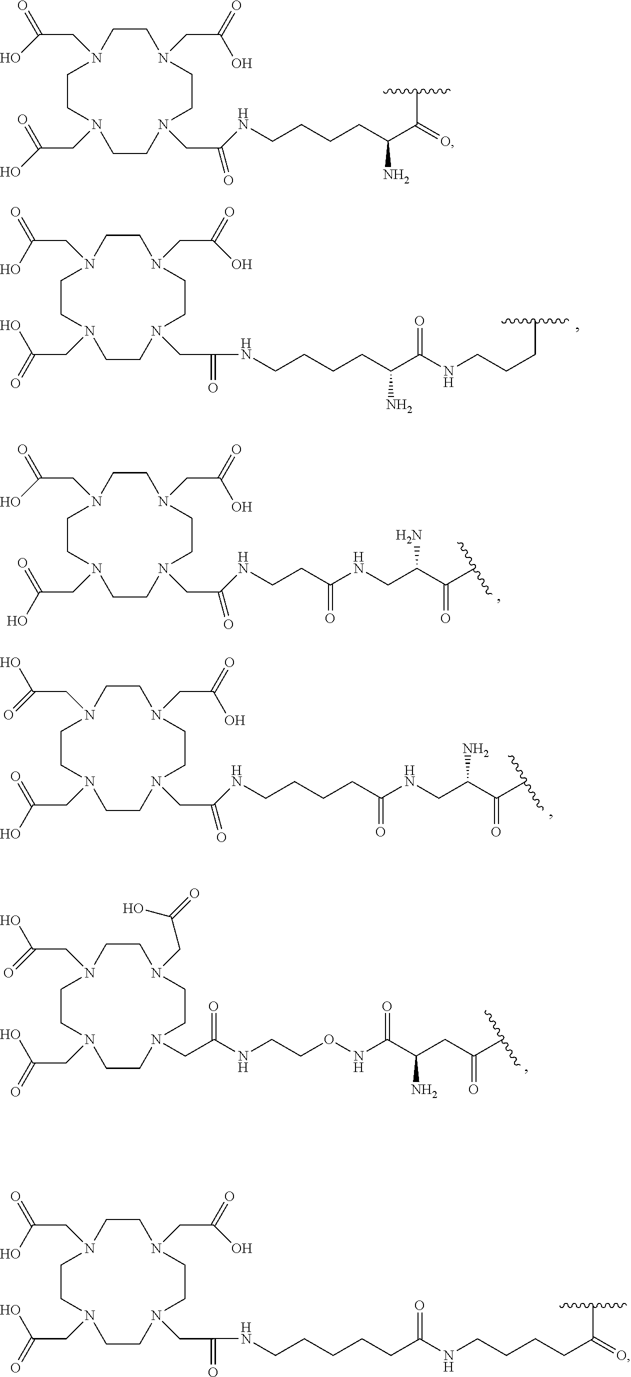

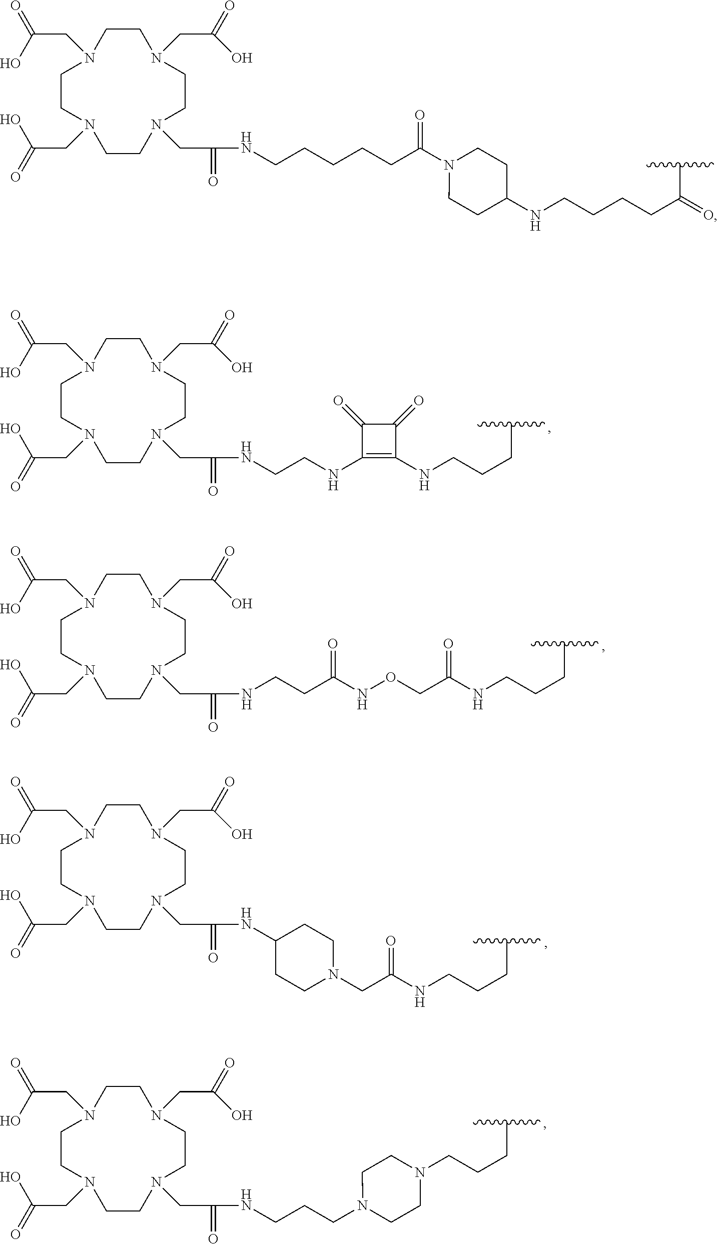 [SMILES: *C(=O)CCCCNC1CCN(C(=O)CCCCCNC(=O)CN2CCN(CC(=O)O)CCN(CC(=O)O)CCN(CC(=O)O)CC2)CC1 ; *CCCNc1c(NCCNC(=O)CN2CCN(CC(=O)O)CCN(CC(=O)O)CCN(CC(=O)O)CC2)c(=O)c1=O ; *CCCNC(=O)CONC(=O)CCNC(=O)CN1CCN(CC(=O)O)CCN(CC(=O)O)CCN(CC(=O)O)CC1 ; *CCCNC(=O)CN1CCC(NC(=O)CN2CCN(CC(=O)O)CCN(CC(=O)O)CCN(CC(=O)O)CC2)CC1 ; *CCCN1CCN(CCCNC(=O)CN2CCN(CC(=O)O)CCN(CC(=O)O)CCN(CC(=O)O)CC2)CC1]

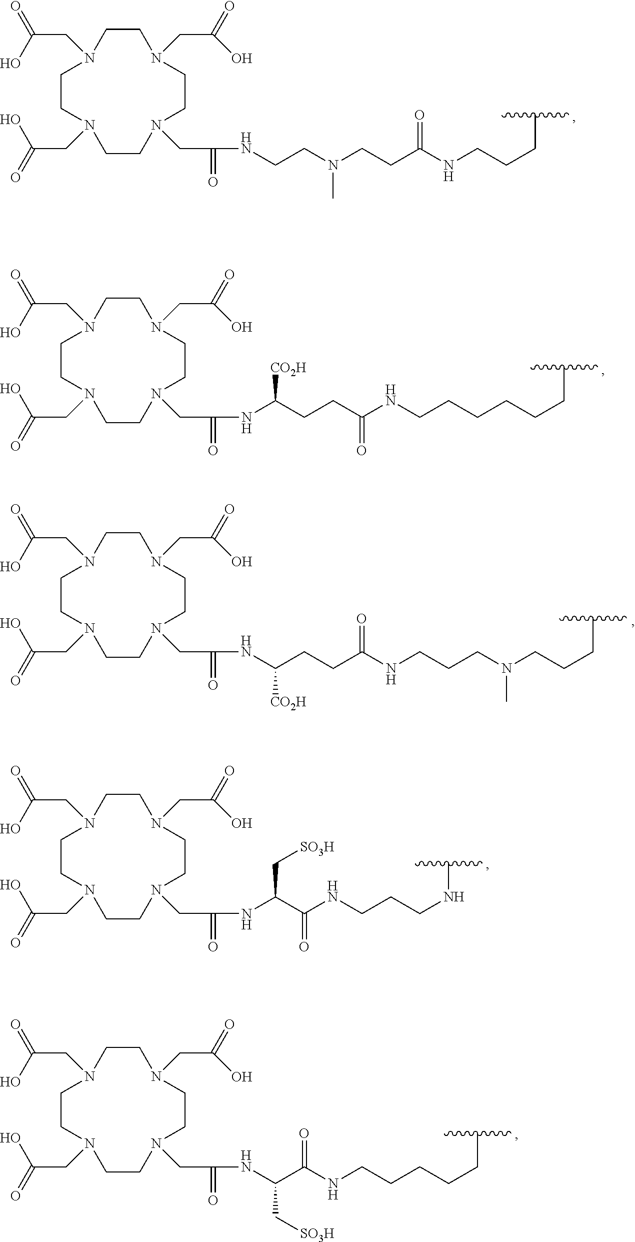 [SMILES: *CCCNC(=O)CCN(C)CCNC(=O)CN1CCN(CC(=O)O)CCN(CC(=O)O)CCN(CC(=O)O)CC1 ; *CCCCCCNC(=O)CC[C@@H](NC(=O)CN1CCN(CC(=O)O)CCN(CC(=O)O)CCN(CC(=O)O)CC1)C(=O)O ; *CCCN(C)CCCNC(=O)CC[C@@H](NC(=O)CN1CCN(CC(=O)O)CCN(CC(=O)O)CCN(CC(=O)O)CC1)C(=O)O ; *NCCCNC(=O)[C@H](CS(=O)(=O)O)NC(=O)CN1CCN(CC(=O)O)CCN(CC(=O)O)CCN(CC(=O)O)CC1 ; *CCCCCNC(=O)[C@H](CS(=O)(=O)O)NC(=O)CN1CCN(CC(=O)O)CCN(CC(=O)O)CCN(CC(=O)O)CC1]

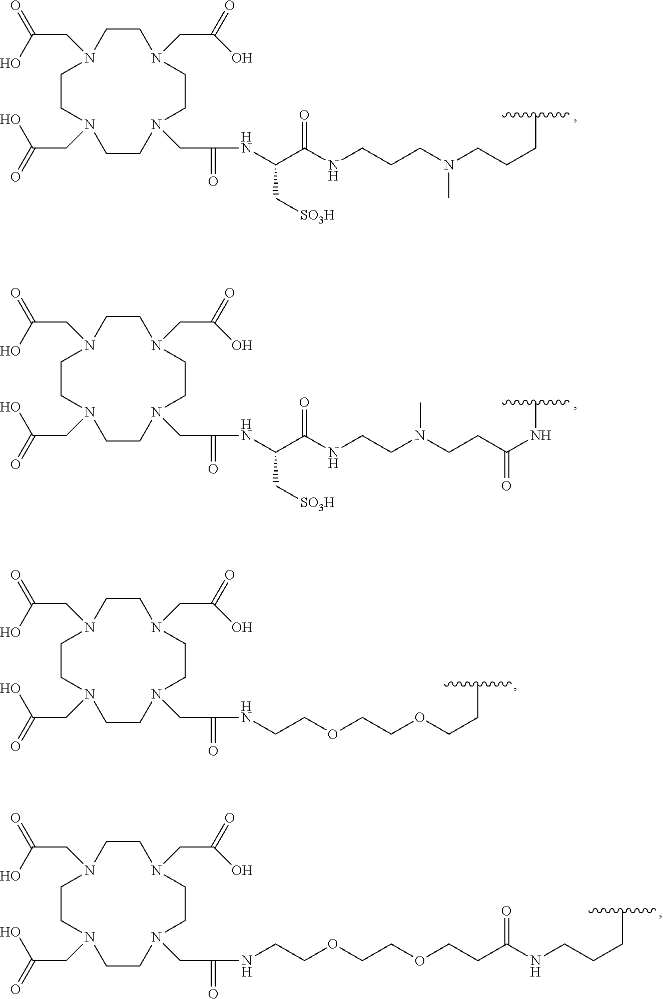 [SMILES: *CCCN(C)CCCNC(=O)[C@H](CS(=O)(=O)O)NC(=O)CN1CCN(CC(=O)O)CCN(CC(=O)O)CCN(CC(=O)O)CC1 ; *NC(=O)CCN(C)CCNC(=O)[C@H](CS(=O)(=O)O)NC(=O)CN1CCN(CC(=O)O)CCN(CC(=O)O)CCN(CC(=O)O)CC1 ; *CCOCCOCCNC(=O)CN1CCN(CC(=O)O)CCN(CC(=O)O)CCN(CC(=O)O)CC1 ; *CCCNC(=O)CCOCCOCCNC(=O)CN1CCN(CC(=O)O)CCN(CC(=O)O)CCN(CC(=O)O)CC1]

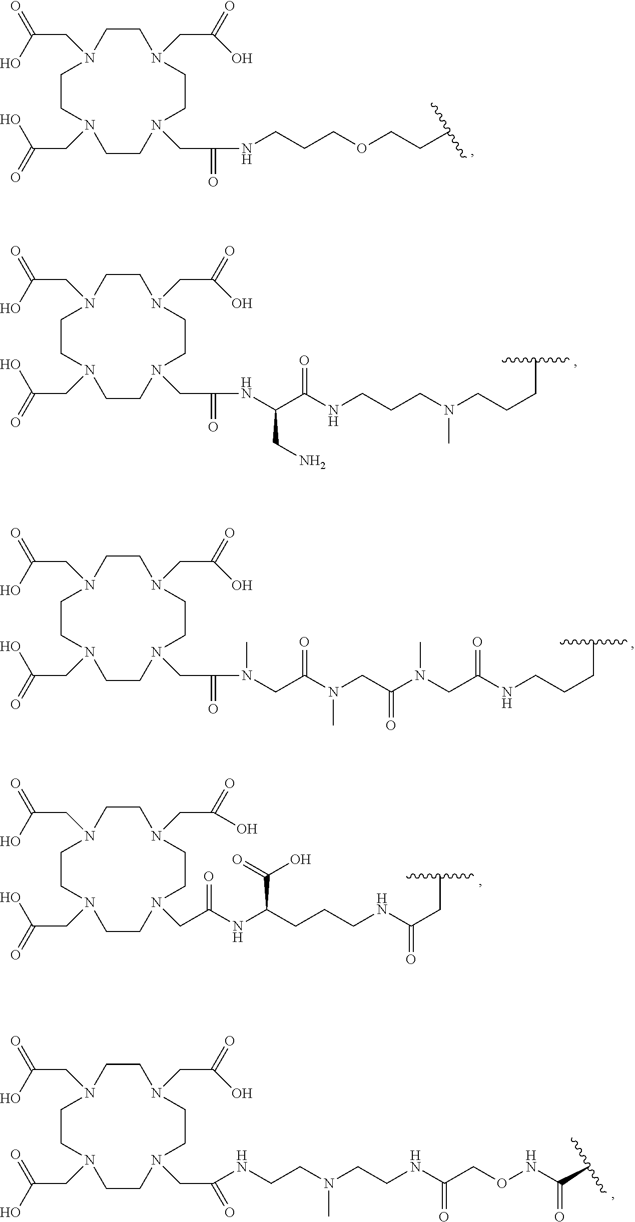

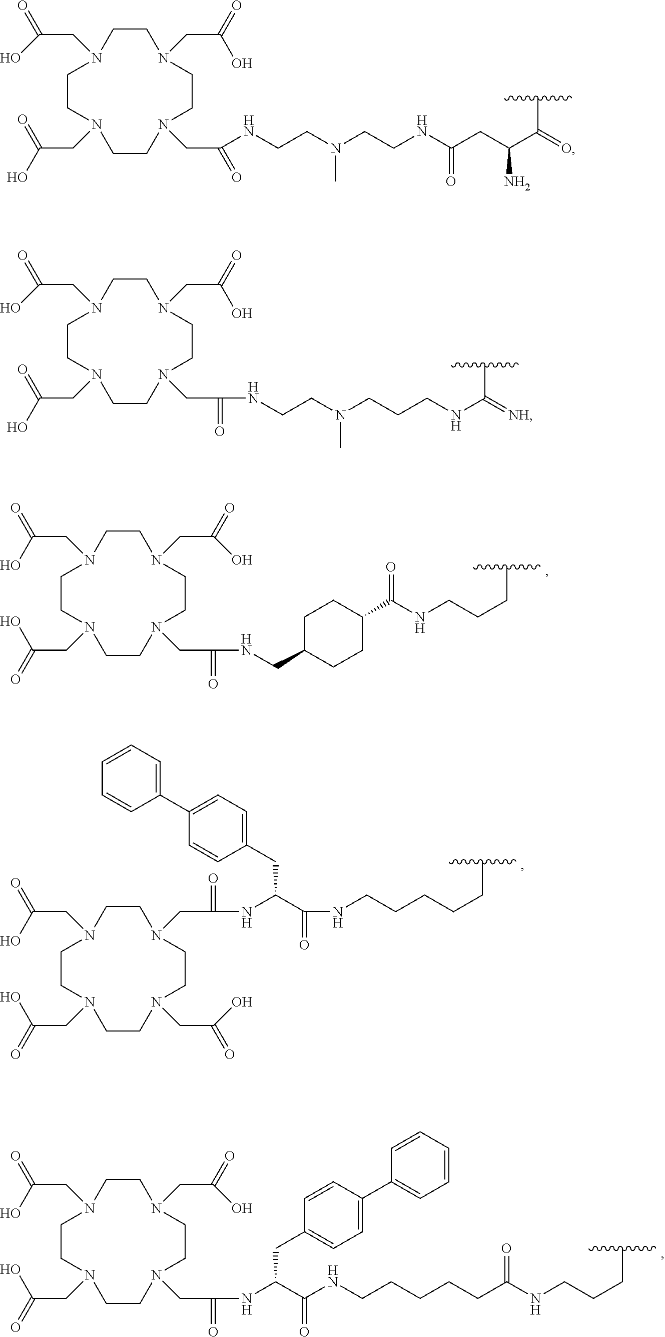 [SMILES: *C(=O)[C@@H](N)CC(=O)NCCN(C)CCNC(=O)CN1CCN(CC(=O)O)CCN(CC(=O)O)CCN(CC(=O)O)CC1 ; *C(=N)NCCCN(C)CCNC(=O)CN1CCN(CC(=O)O)CCN(CC(=O)O)CCN(CC(=O)O)CC1 ; *CCCNC(=O)[C@H]1CC[C@H](CNC(=O)CN2CCN(CC(=O)O)CCN(CC(=O)O)CCN(CC(=O)O)CC2)CC1 ; *CCCCCNC(=O)[C@@H](Cc1ccc(-c2ccccc2)cc1)NC(=O)CN1CCN(CC(=O)O)CCN(CC(=O)O)CCN(CC(=O)O)CC1 ; *CCCNC(=O)CCCCCNC(=O)[C@@H](Cc1ccc(-c2ccccc2)cc1)NC(=O)CN1CCN(CC(=O)O)CCN(CC(=O)O)CCN(CC(=O)O)CC1]

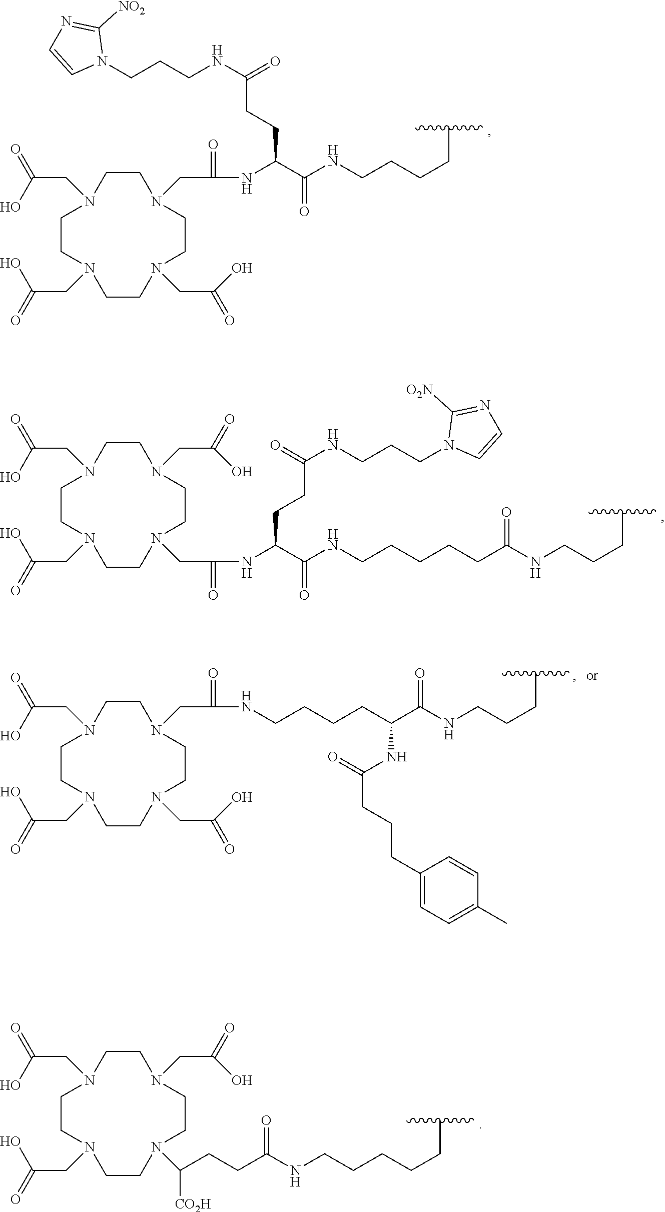 [SMILES: *CCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)CCCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)[C@@H](CCCCNC(=O)CN1CCN(CC(=O)O)CCN(CC(=O)O)CCN(CC(=O)O)CC1)NC(=O)CCCc1ccc(C)cc1 ; *CCCCCNC(=O)CCC(C(=O)O)N1CCN(CC(=O)O)CCN(CC(=O)O)CCN(CC(=O)O)CC1]

In some embodiments, R^{a}-L- is

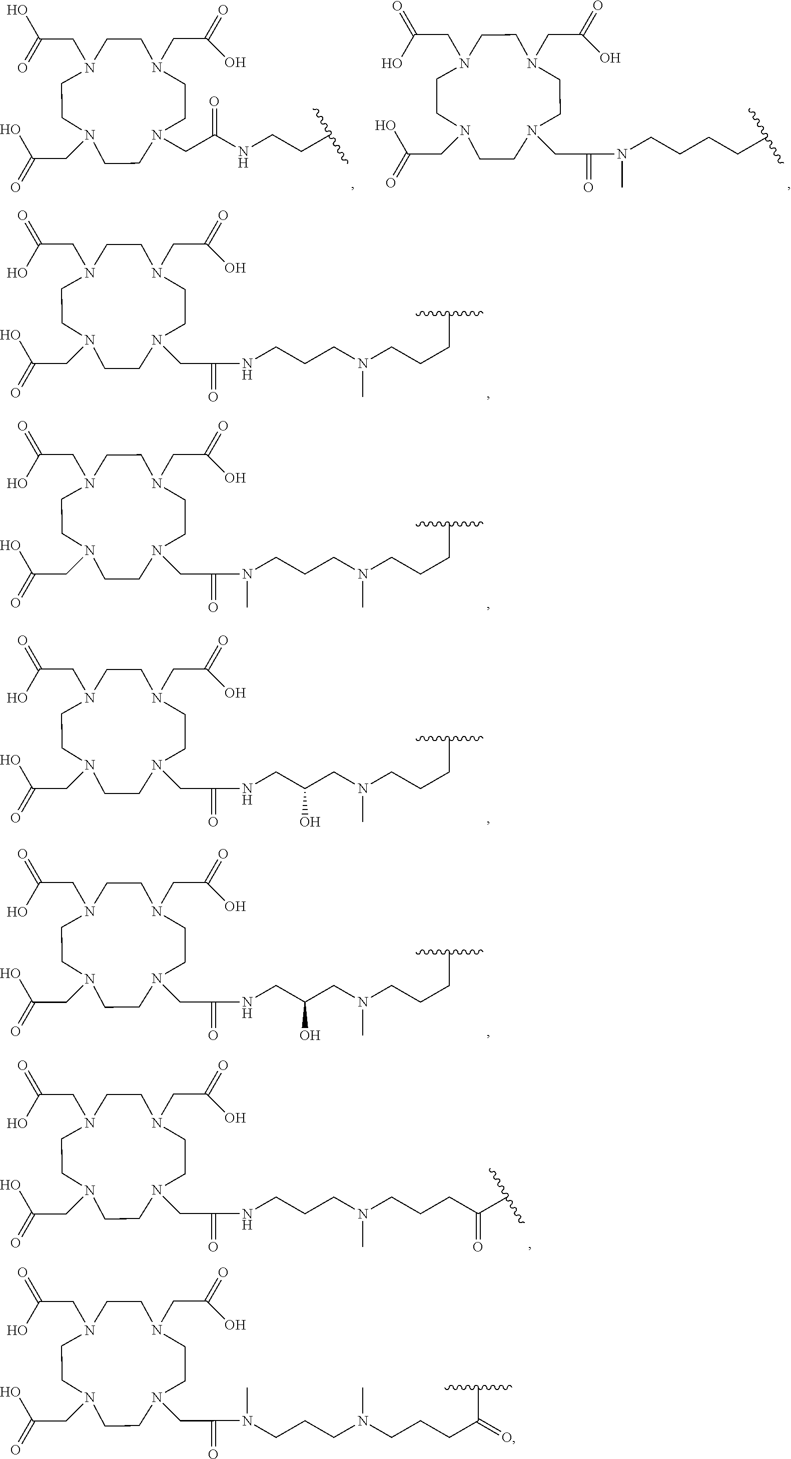

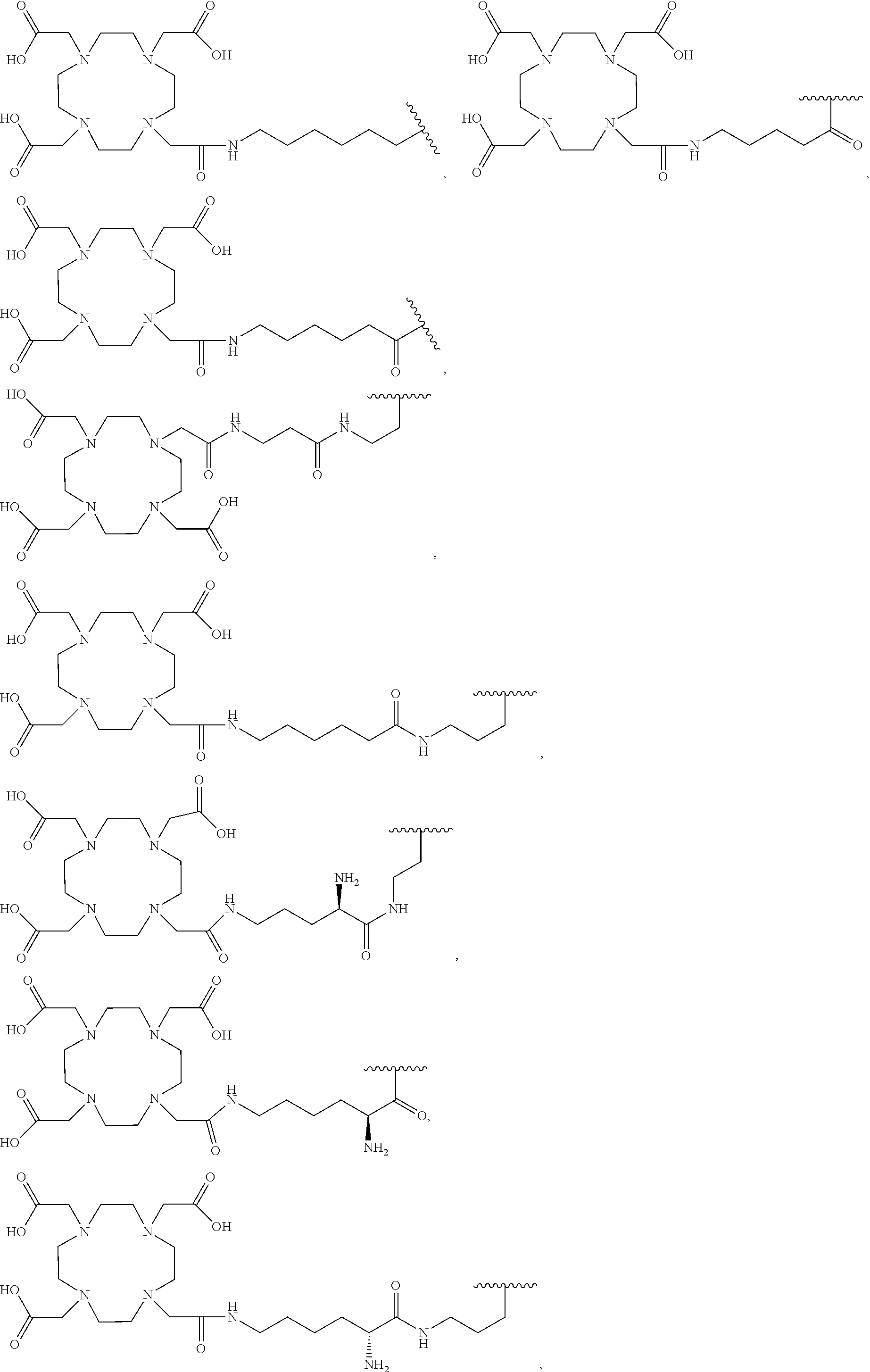

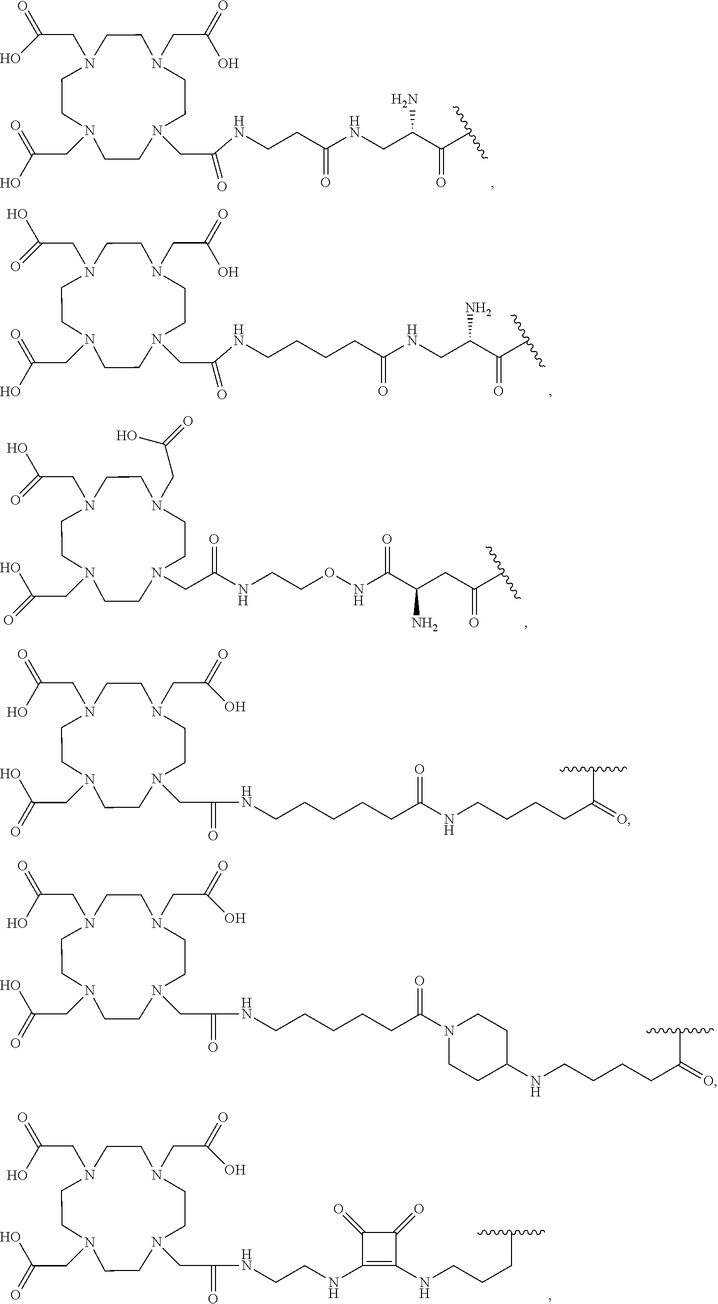

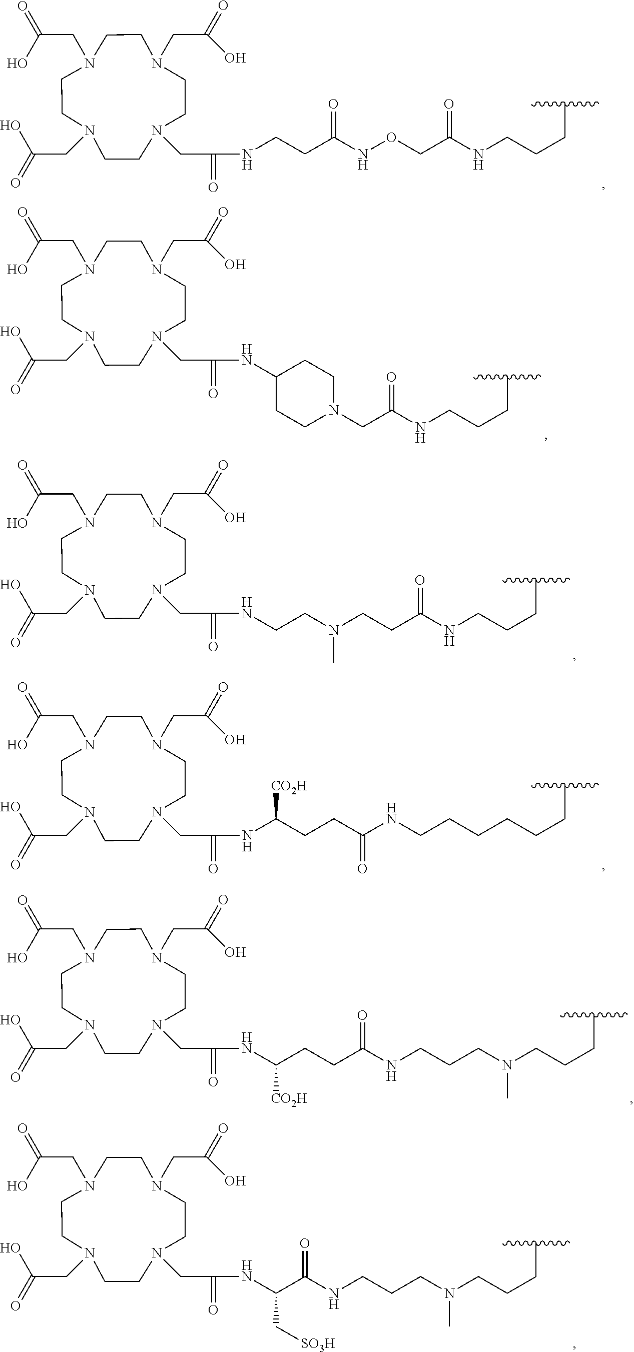

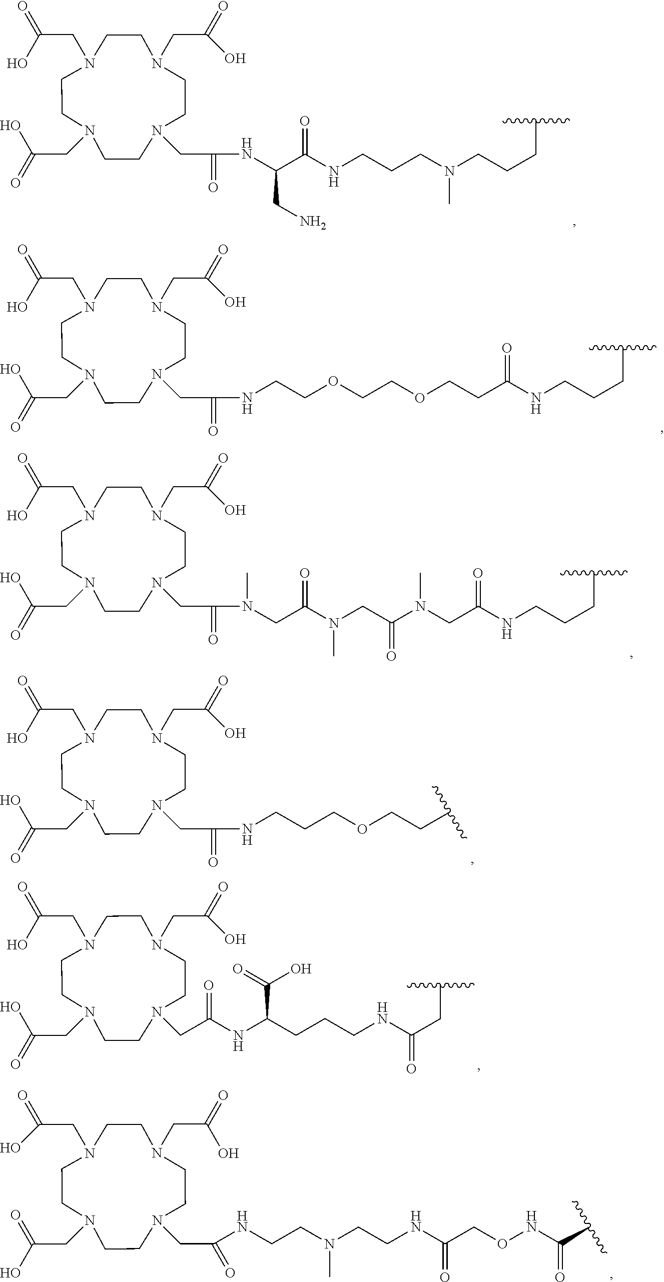

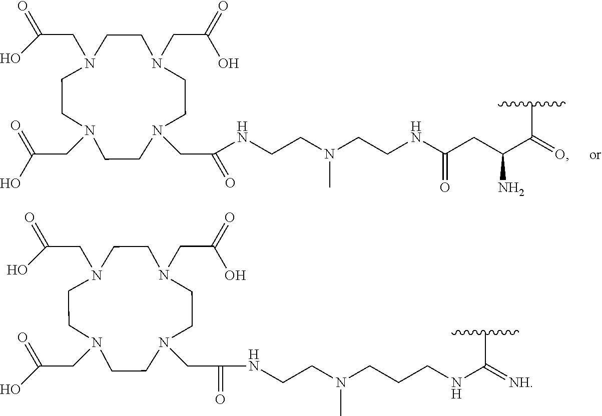

In some embodiments, R^{a}-L- is

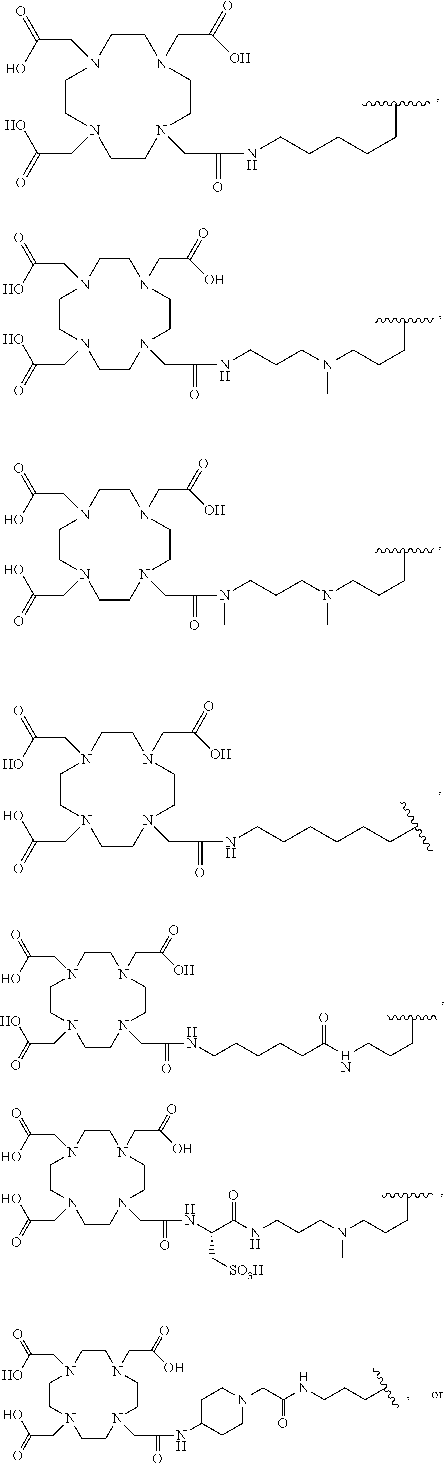

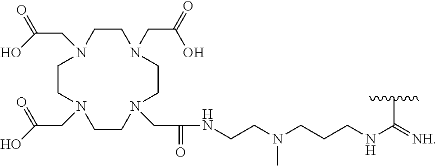 [SMILES: *C(=N)NCCCN(C)CCNC(=O)CN1CCN(CC(=O)O)CCN(CC(=O)O)CCN(CC(=O)O)CC1]

In some embodiments, R^{a}-L- is

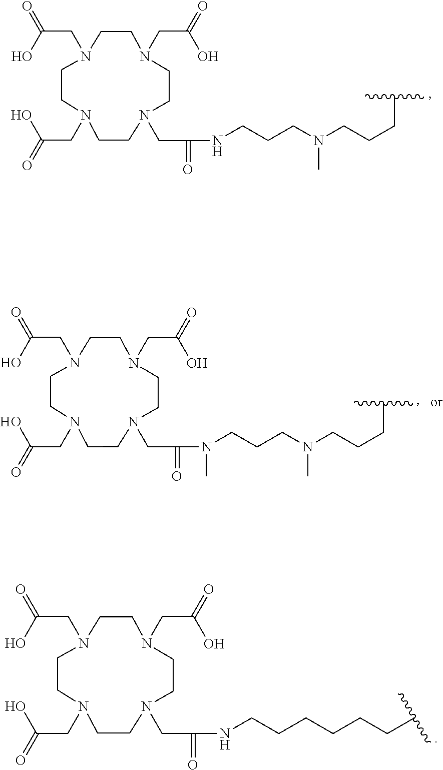 [SMILES: *CCCN(C)CCCNC(=O)CN1CCN(CC(=O)O)CCN(CC(=O)O)CCN(CC(=O)O)CC1 ; *CCCN(C)CCCN(C)C(=O)CN1CCN(CC(=O)O)CCN(CC(=O)O)CCN(CC(=O)O)CC1 ; *CCCCCCNC(=O)CN1CCN(CC(=O)O)CCN(CC(=O)O)CCN(CC(=O)O)CC1]

### Representative Conjugate Compounds

Representative NTSR1 radiopharmaceuticals described herein have one of the following structures. or a pharmaceutically acceptable salt thereof:

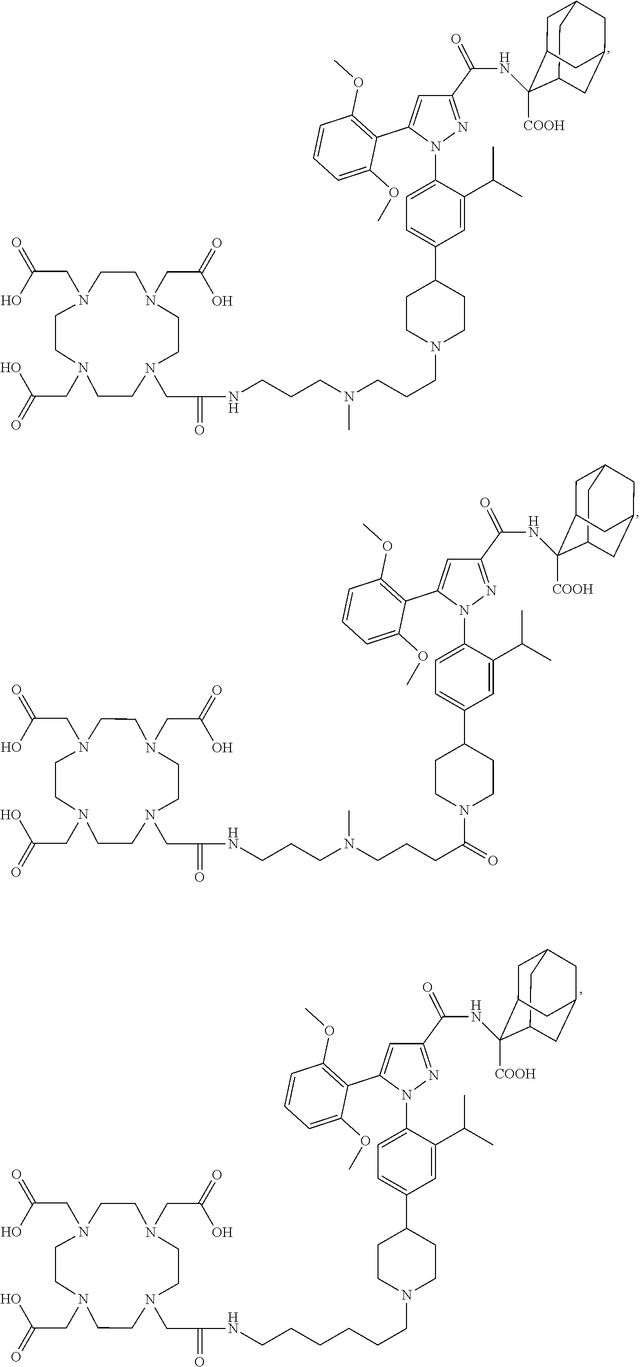 [SMILES: COc1cccc(OC)c1-c1cc(C(=O)NC2(C(=O)O)C3CC4CC(C3)CC2C4)nn1-c1ccc(C2CCN(CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCCCNC(=O)CN3CCN(CC(=O)O)CCN(CC(=O)O)CCN(CC(=O)O)CC3)CC2)cc1C(C)C]

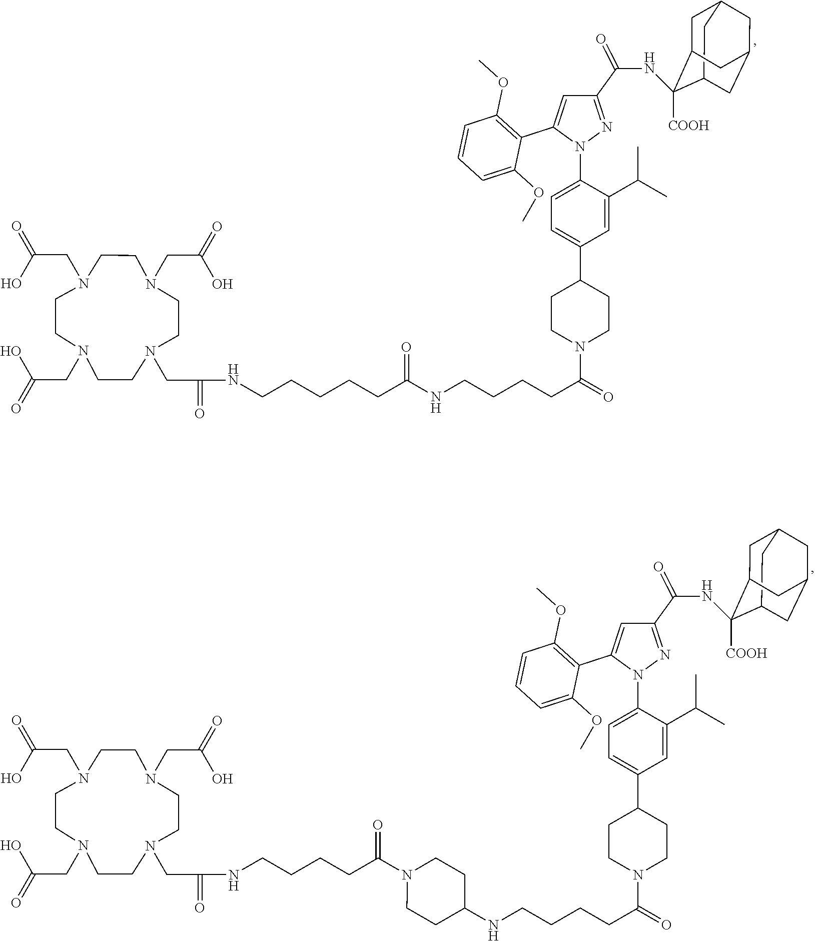 [SMILES: COc1cccc(OC)c1-c1cc(C(=O)NC2(C(=O)O)C3CC4CC(C3)CC2C4)nn1-c1ccc(C2CCN(C(=O)CCCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCCNC3CCN(C(=O)CCCCNC(=O)CN4CCN(CC(=O)O)CCN(CC(=O)O)CCN(CC(=O)O)CC4)CC3)CC2)cc1C(C)C]

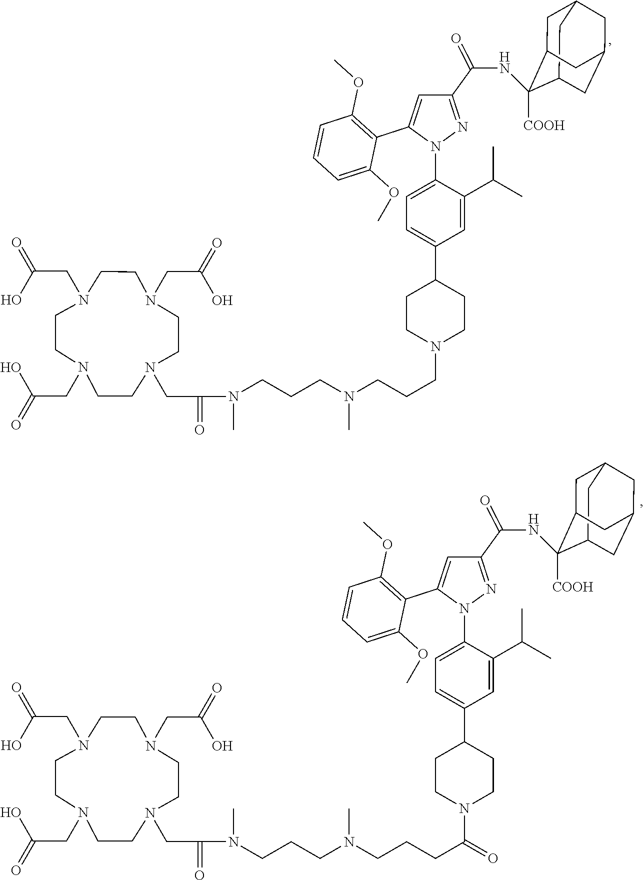 [SMILES: COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCN(C)CCCN(C)C(=O)CN3CCN(CC(=O)O)CCN(CC(=O)O)CCN(CC(=O)O)CC3)CC2)cc1C(C)C]

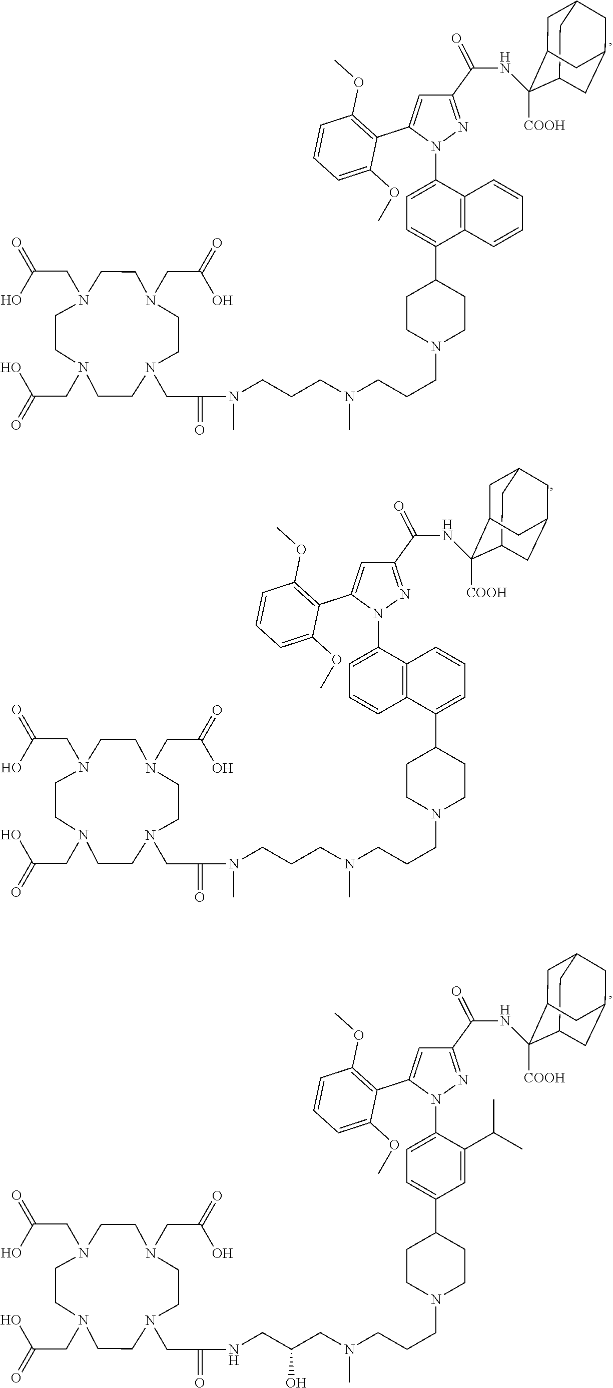 [SMILES: COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)c2ccccc12 ; COc1cccc(OC)c1-c1cc(C(=O)NC2(C(=O)O)C3CC4CC(C3)CC2C4)nn1-c1cccc2c(C3CCN(CCCN(C)CCCN(C)C(=O)CN4CCN(CC(=O)O)CCN(CC(=O)O)CCN(CC(=O)O)CC4)CC3)cccc12 ; COc1cccc(OC)c1-c1cc(C(=O)NC2(C(=O)O)C3CC4CC(C3)CC2C4)nn1-c1ccc(C2CCN(CCCN(C)C[C@H](O)CNC(=O)CN3CCN(CC(=O)O)CCN(CC(=O)O)CCN(CC(=O)O)CC3)CC2)cc1C(C)C]

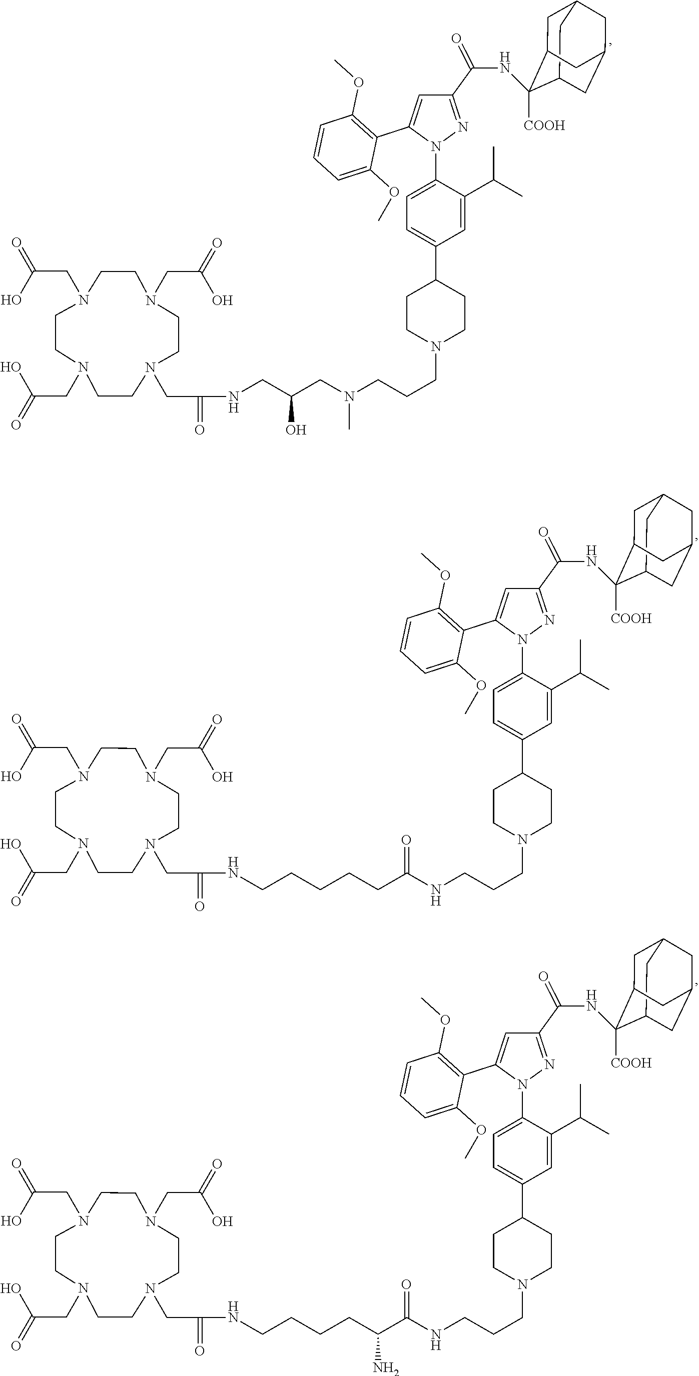 [SMILES: COc1cccc(OC)c1-c1cc(C(=O)NC2(C(=O)O)C3CC4CC(C3)CC2C4)nn1-c1ccc(C2CCN(CCCN(C)C[C@@H](O)CNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)[C@H](N)CCCCNC(=O)CN3CCN(CC(=O)O)CCN(CC(=O)O)CCN(CC(=O)O)CC3)CC2)cc1C(C)C]

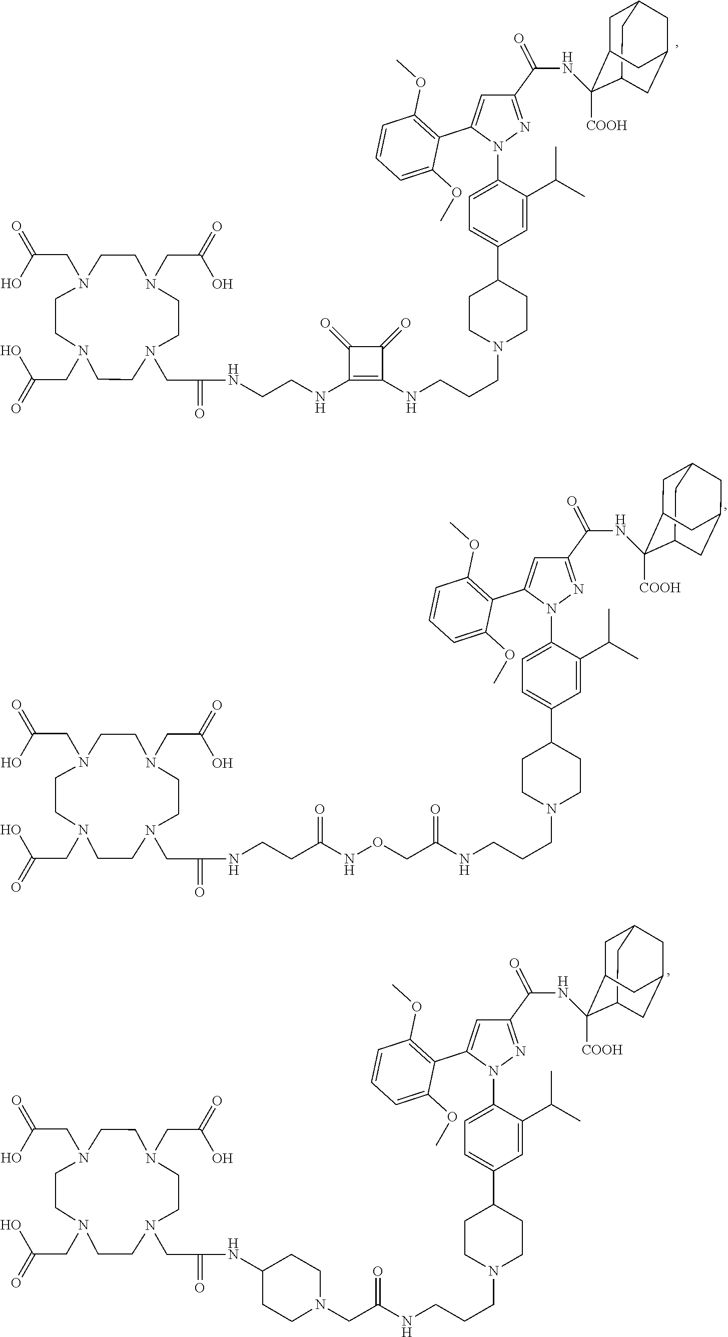 [SMILES: COc1cccc(OC)c1-c1cc(C(=O)NC2(C(=O)O)C3CC4CC(C3)CC2C4)nn1-c1ccc(C2CCN(CCCNc3c(NCCNC(=O)CN4CCN(CC(=O)O)CCN(CC(=O)O)CCN(CC(=O)O)CC4)c(=O)c3=O)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CONC(=O)CCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CN3CCC(NC(=O)CN4CCN(CC(=O)O)CCN(CC(=O)O)CCN(CC(=O)O)CC4)CC3)CC2)cc1C(C)C]

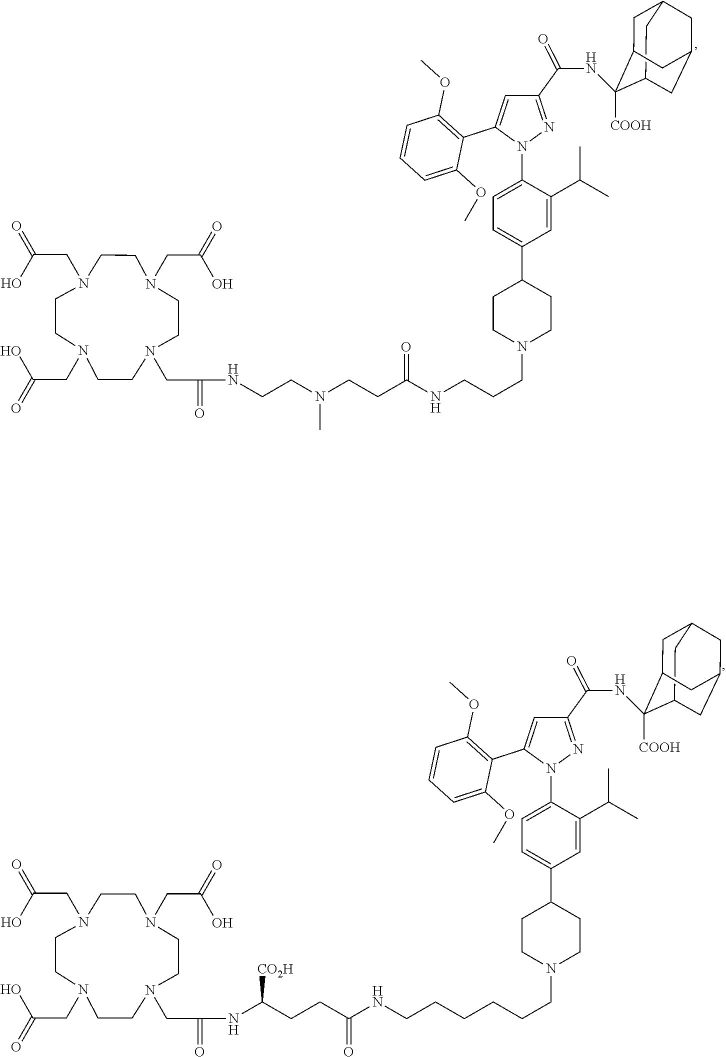 [SMILES: COc1cccc(OC)c1-c1cc(C(=O)NC2(C(=O)O)C3CC4CC(C3)CC2C4)nn1-c1ccc(C2CCN(CCCNC(=O)CCN(C)CCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCCCNC(=O)CC[C@@H](NC(=O)CN3CCN(CC(=O)O)CCN(CC(=O)O)CCN(CC(=O)O)CC3)C(=O)O)CC2)cc1C(C)C]

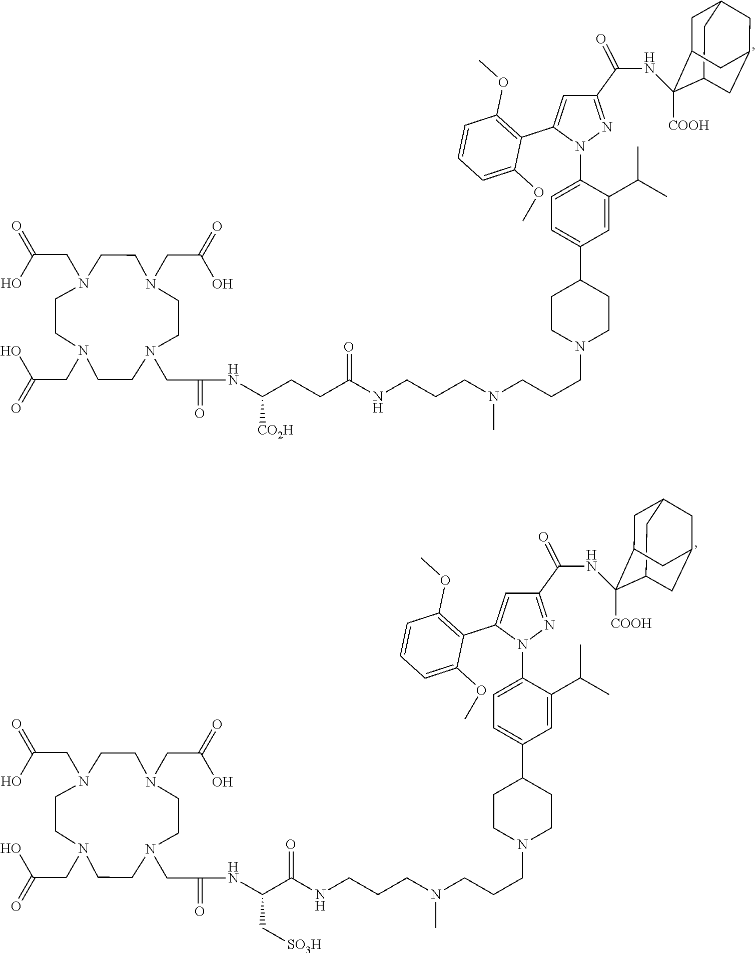 [SMILES: COc1cccc(OC)c1-c1cc(C(=O)NC2(C(=O)O)C3CC4CC(C3)CC2C4)nn1-c1ccc(C2CCN(CCCN(C)CCCNC(=O)CC[C@@H](NC(=O)CN3CCN(CC(=O)O)CCN(CC(=O)O)CCN(CC(=O)O)CC3)C(=O)O)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C]

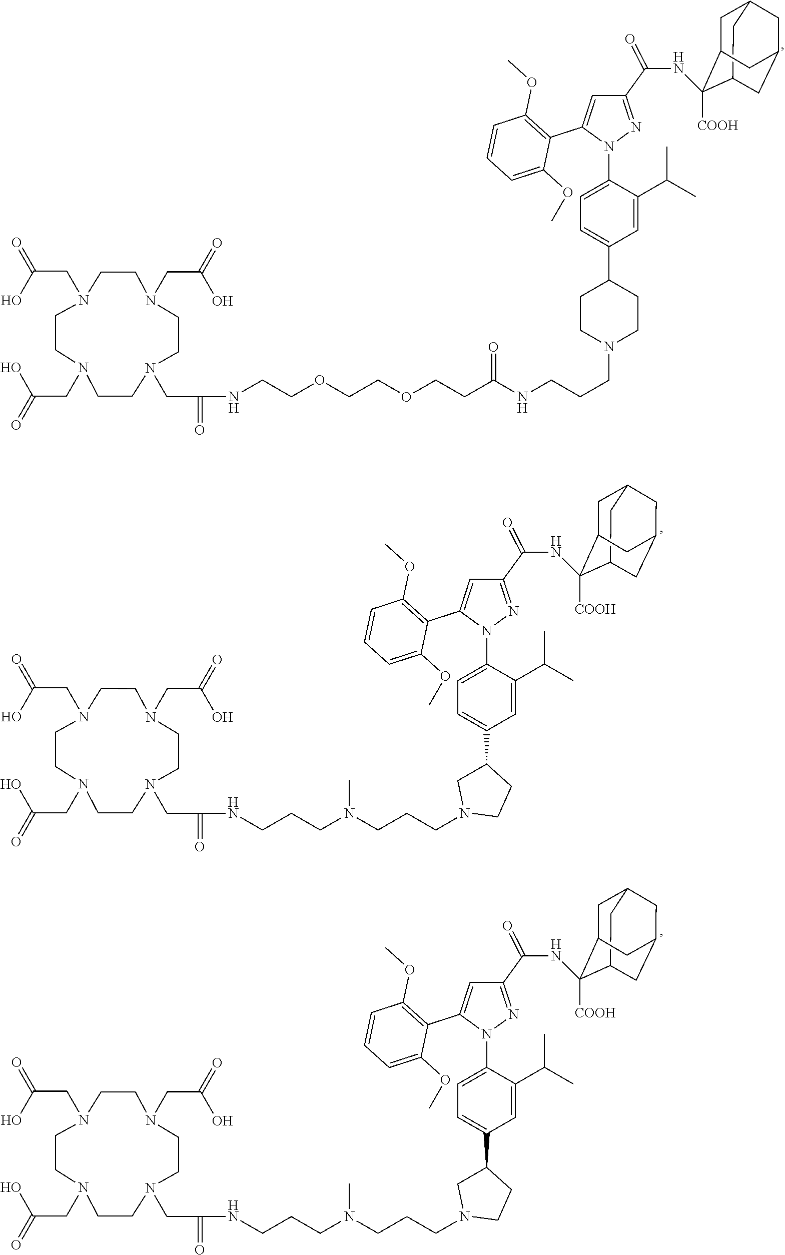 [SMILES: COc1cccc(OC)c1-c1cc(C(=O)NC2(C(=O)O)C3CC4CC(C3)CC2C4)nn1-c1ccc(C2CCN(CCCNC(=O)CCOCCOCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc([C@@H]2CCN(CCCN(C)CCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc([C@H]2CCN(CCCN(C)CCCNC(=O)CN3CCN(CC(=O)O)CCN(CC(=O)O)CCN(CC(=O)O)CC3)C2)cc1C(C)C]

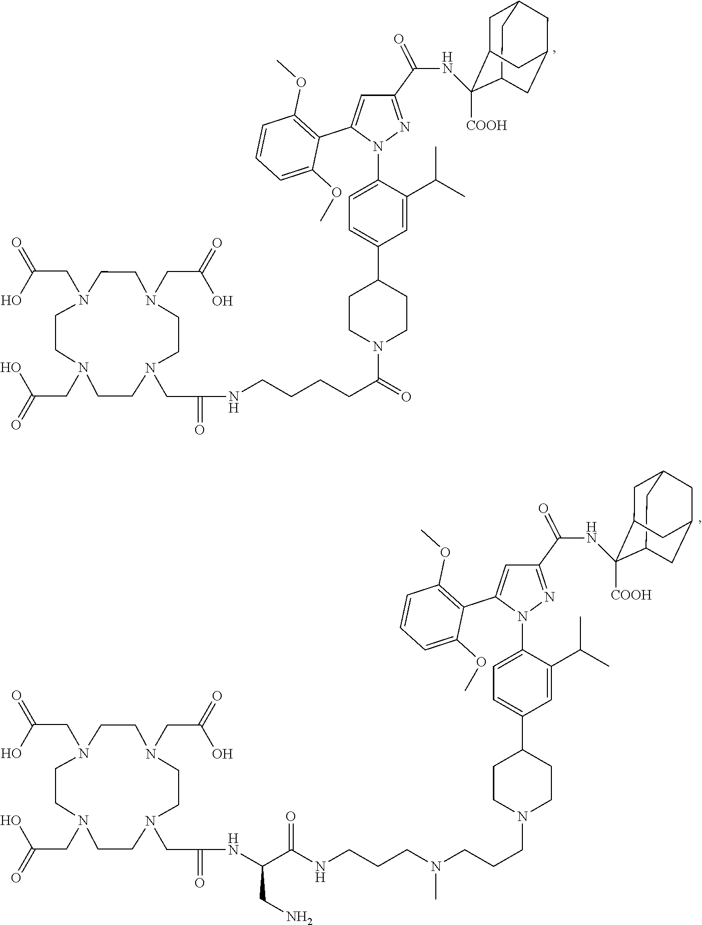 [SMILES: COc1cccc(OC)c1-c1cc(C(=O)NC2(C(=O)O)C3CC4CC(C3)CC2C4)nn1-c1ccc(C2CCN(C(=O)CCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)[C@@H](CN)NC(=O)CN3CCN(CC(=O)O)CCN(CC(=O)O)CCN(CC(=O)O)CC3)CC2)cc1C(C)C]

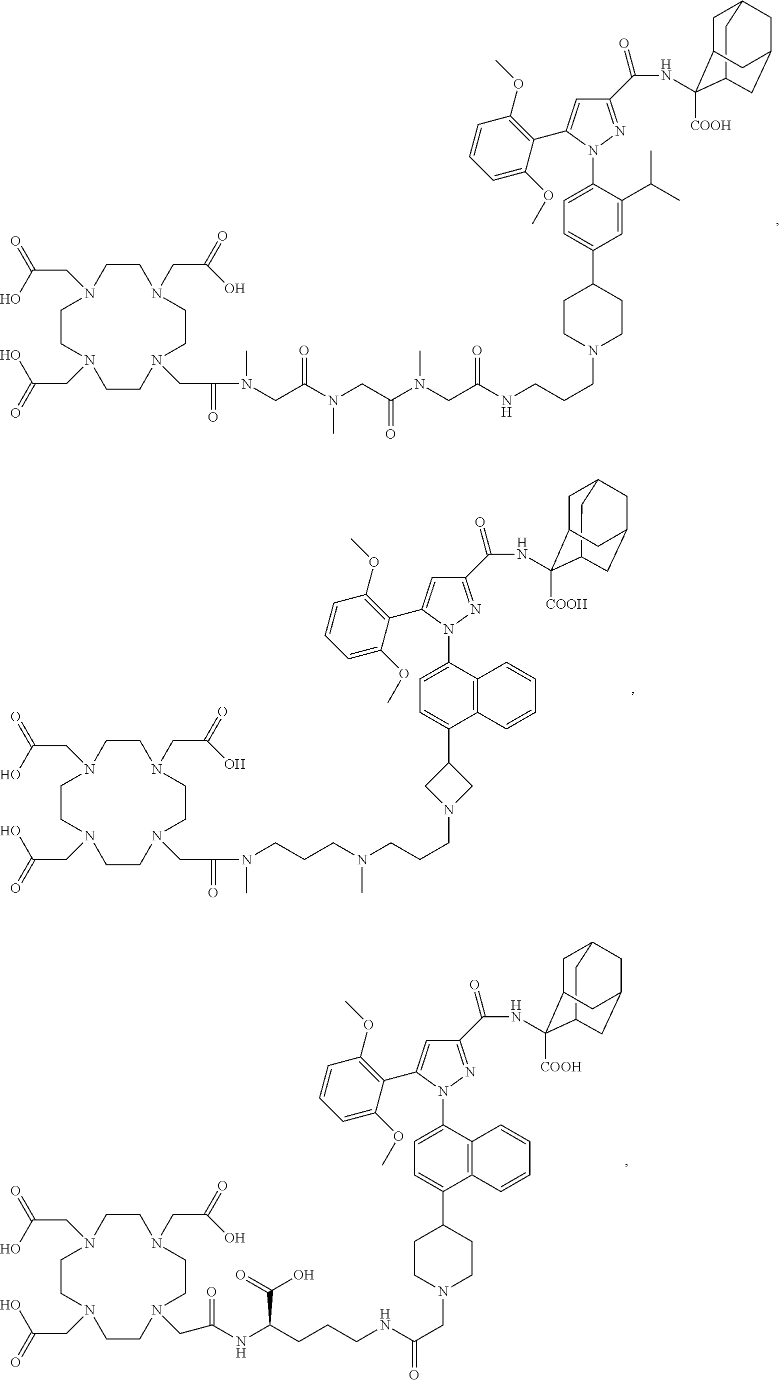 [SMILES: COc1cccc(OC)c1-c1cc(C(=O)NC2(C(=O)O)C3CC4CC(C3)CC2C4)nn1-c1ccc(C2CCN(CCCNC(=O)CN(C)C(=O)CN(C)C(=O)CN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CN(CCCN(C)CCCN(C)C(=O)CN3CCN(CC(=O)O)CCN(CC(=O)O)CCN(CC(=O)O)CC3)C2)c2ccccc12 ; COc1cccc(OC)c1-c1cc(C(=O)NC2(C(=O)O)C3CC4CC(C3)CC2C4)nn1-c1ccc(C2CCN(CC(=O)NCCC[C@@H](NC(=O)CN3CCN(CC(=O)O)CCN(CC(=O)O)CCN(CC(=O)O)CC3)C(=O)O)CC2)c2ccccc12]

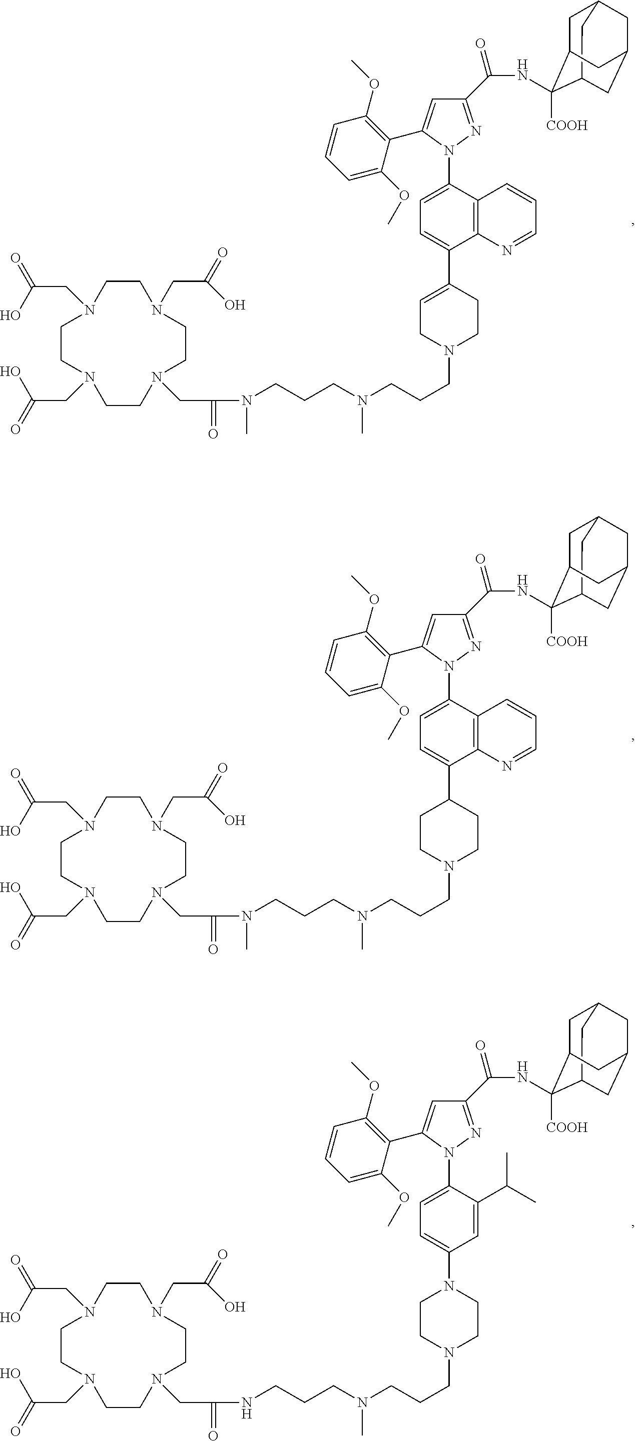 [SMILES: COc1cccc(OC)c1-c1cc(C(=O)NC2(C(=O)O)C3CC4CC(C3)CC2C4)nn1-c1ccc(C2=CCN(CCCN(C)CCCN(C)C(=O)CN3CCN(CC(=O)O)CCN(CC(=O)O)CCN(CC(=O)O)CC3)CC2)c2ncccc12 ; COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)c2ncccc12 ; COc1cccc(OC)c1-c1cc(C(=O)NC2(C(=O)O)C3CC4CC(C3)CC2C4)nn1-c1ccc(N2CCN(CCCN(C)CCCNC(=O)CN3CCN(CC(=O)O)CCN(CC(=O)O)CCN(CC(=O)O)CC3)CC2)cc1C(C)C]

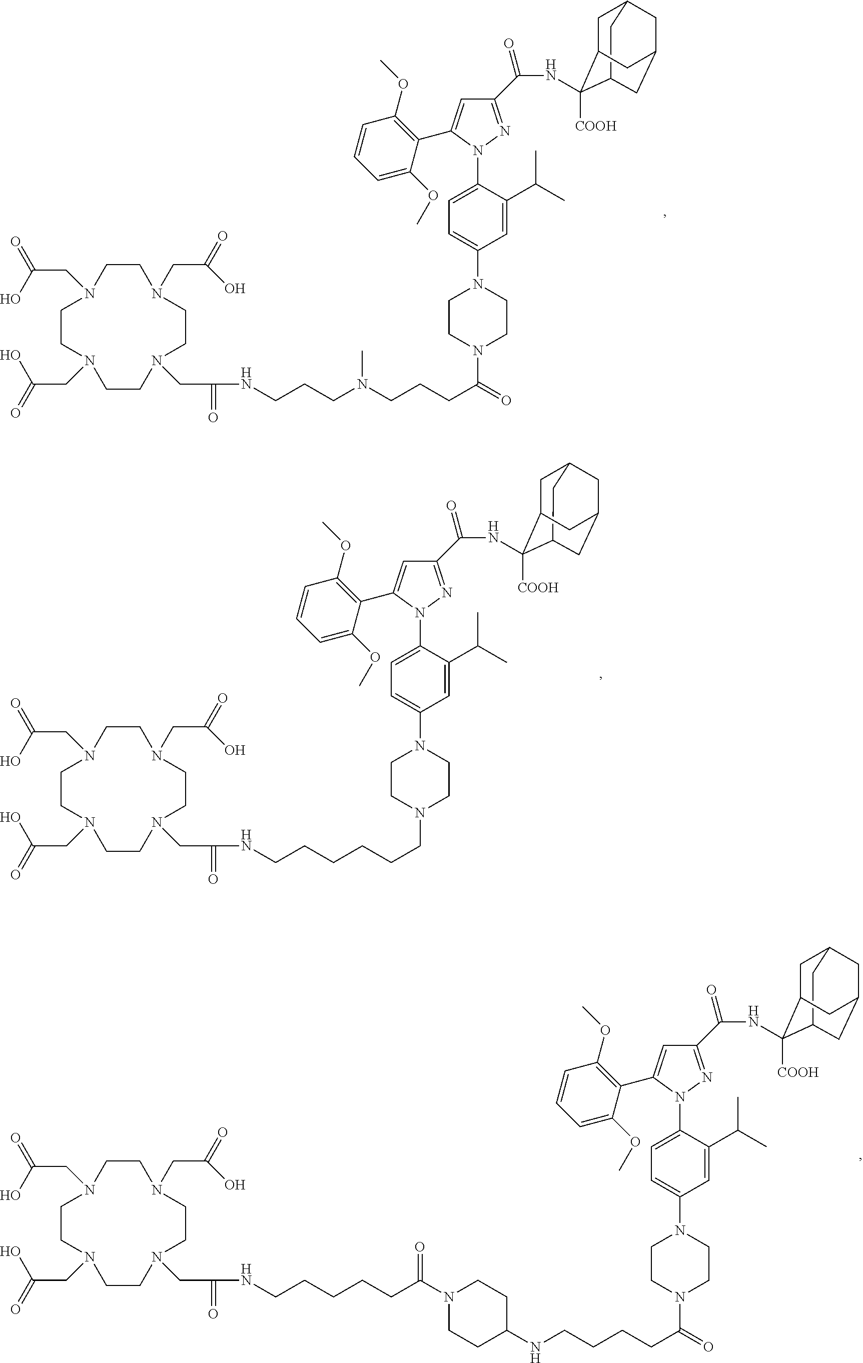 [SMILES: COc1cccc(OC)c1-c1cc(C(=O)NC2(C(=O)O)C3CC4CC(C3)CC2C4)nn1-c1ccc(N2CCN(C(=O)CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCN(CCCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCN(C(=O)CCCCNC3CCN(C(=O)CCCCCNC(=O)CN4CCN(CC(=O)O)CCN(CC(=O)O)CCN(CC(=O)O)CC4)CC3)CC2)cc1C(C)C]

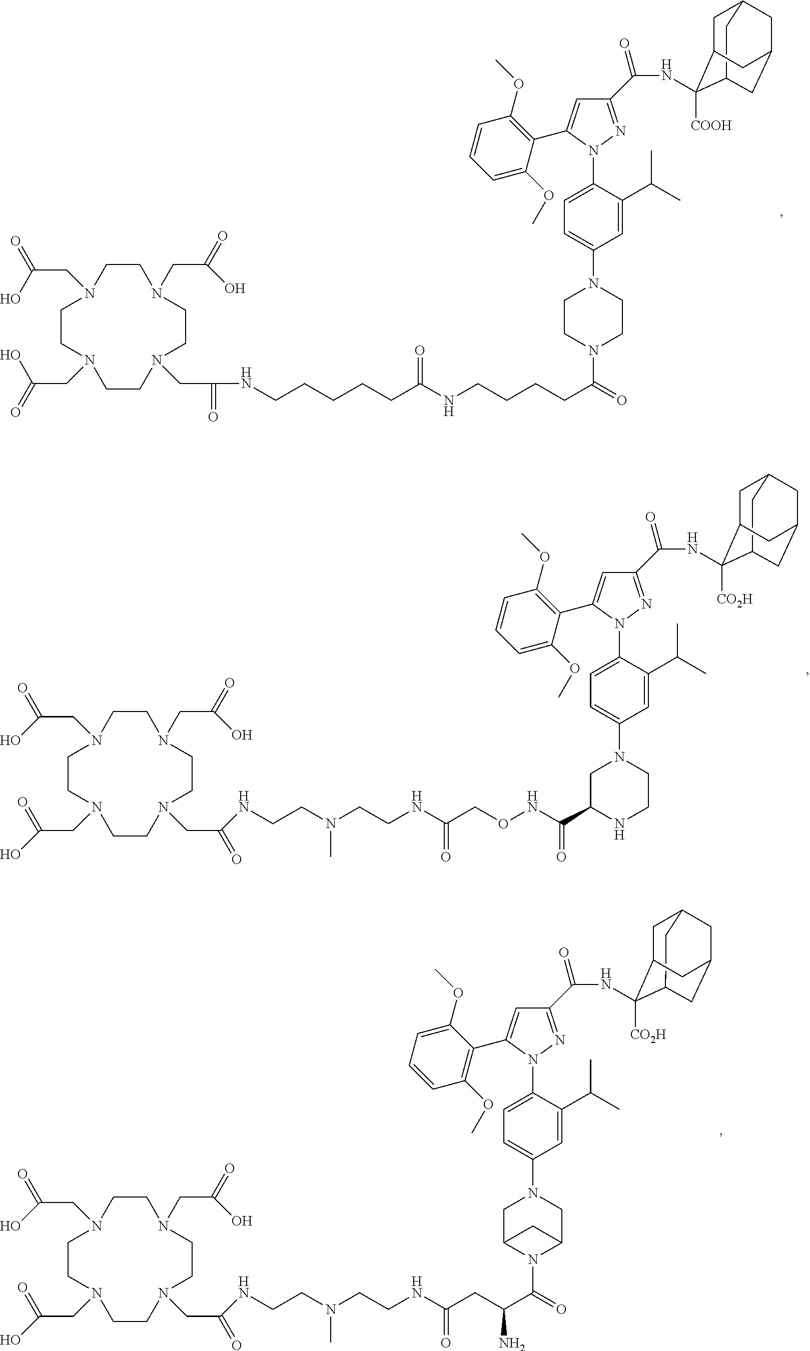 [SMILES: COc1cccc(OC)c1-c1cc(C(=O)NC2(C(=O)O)C3CC4CC(C3)CC2C4)nn1-c1ccc(N2CCN(C(=O)CCCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCN[C@@H](C(=O)NOCC(=O)NCCN(C)CCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(N2CC3CC(C2)N3C(=O)[C@@H](N)CC(=O)NCCN(C)CCNC(=O)CN2CCN(CC(=O)O)CCN(CC(=O)O)CCN(CC(=O)O)CC2)cc1C(C)C]

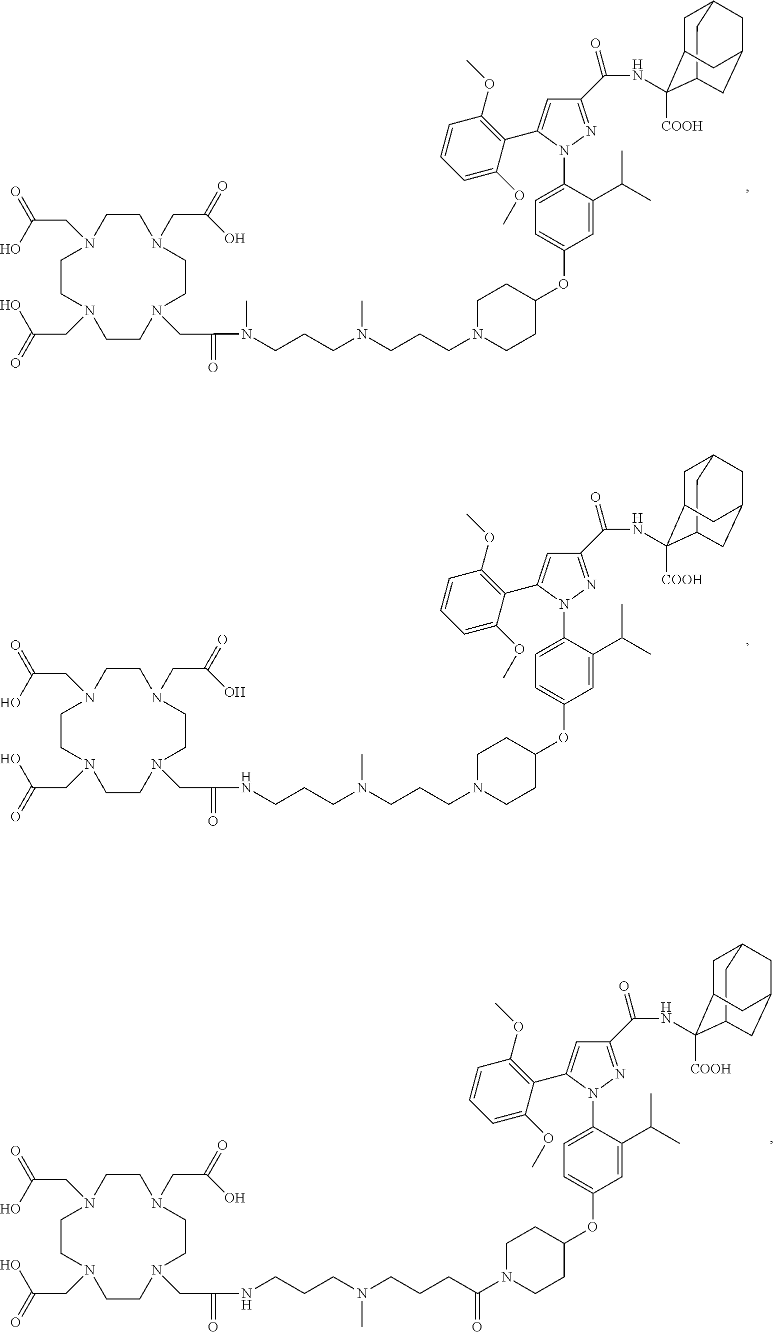 [SMILES: COc1cccc(OC)c1-c1cc(C(=O)NC2(C(=O)O)C3CC4CC(C3)CC2C4)nn1-c1ccc(OC2CCN(CCCN(C)CCCN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(C(=O)CCCN(C)CCCNC(=O)CN3CCN(CC(=O)O)CCN(CC(=O)O)CCN(CC(=O)O)CC3)CC2)cc1C(C)C]

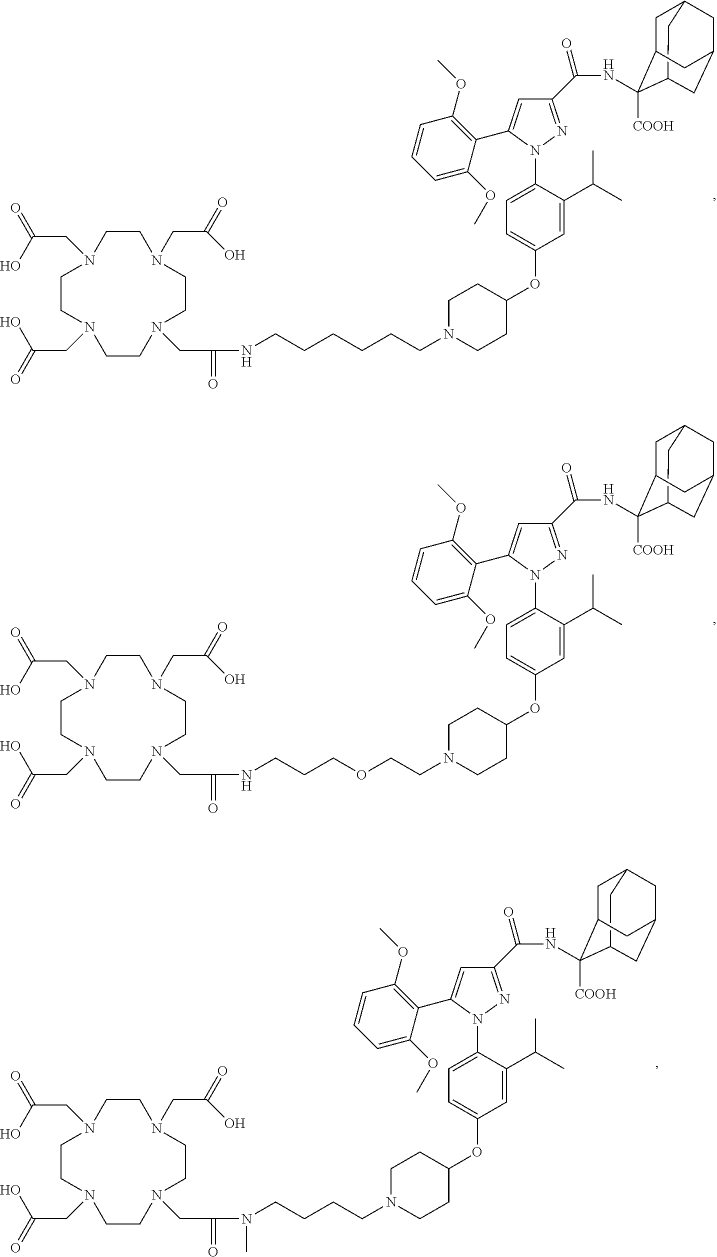 [SMILES: COc1cccc(OC)c1-c1cc(C(=O)NC2(C(=O)O)C3CC4CC(C3)CC2C4)nn1-c1ccc(OC2CCN(CCCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(CCOCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(CCCCN(C)C(=O)CN3CCN(CC(=O)O)CCN(CC(=O)O)CCN(CC(=O)O)CC3)CC2)cc1C(C)C]

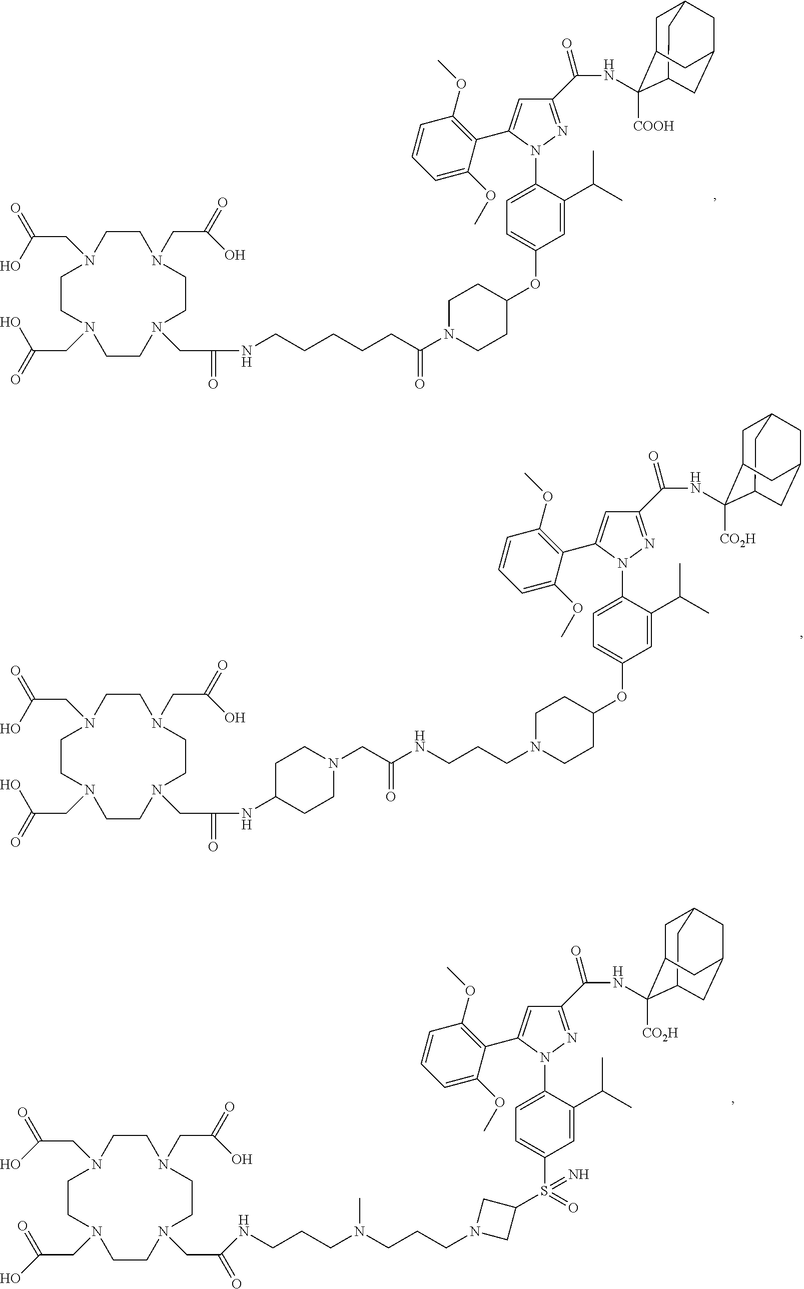 [SMILES: COc1cccc(OC)c1-c1cc(C(=O)NC2(C(=O)O)C3CC4CC(C3)CC2C4)nn1-c1ccc(OC2CCN(C(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(CCCNC(=O)CN3CCC(NC(=O)CN4CCN(CC(=O)O)CCN(CC(=O)O)CCN(CC(=O)O)CC4)CC3)CC2)cc1C(C)C ; COc1cccc(OC)c1-c1cc(C(=O)NC2(C(=O)O)C3CC4CC(C3)CC2C4)nn1-c1ccc(S(=N)(=O)C2CN(CCCN(C)CCCNC(=O)CN3CCN(CC(=O)O)CCN(CC(=O)O)CCN(CC(=O)O)CC3)C2)cc1C(C)C]

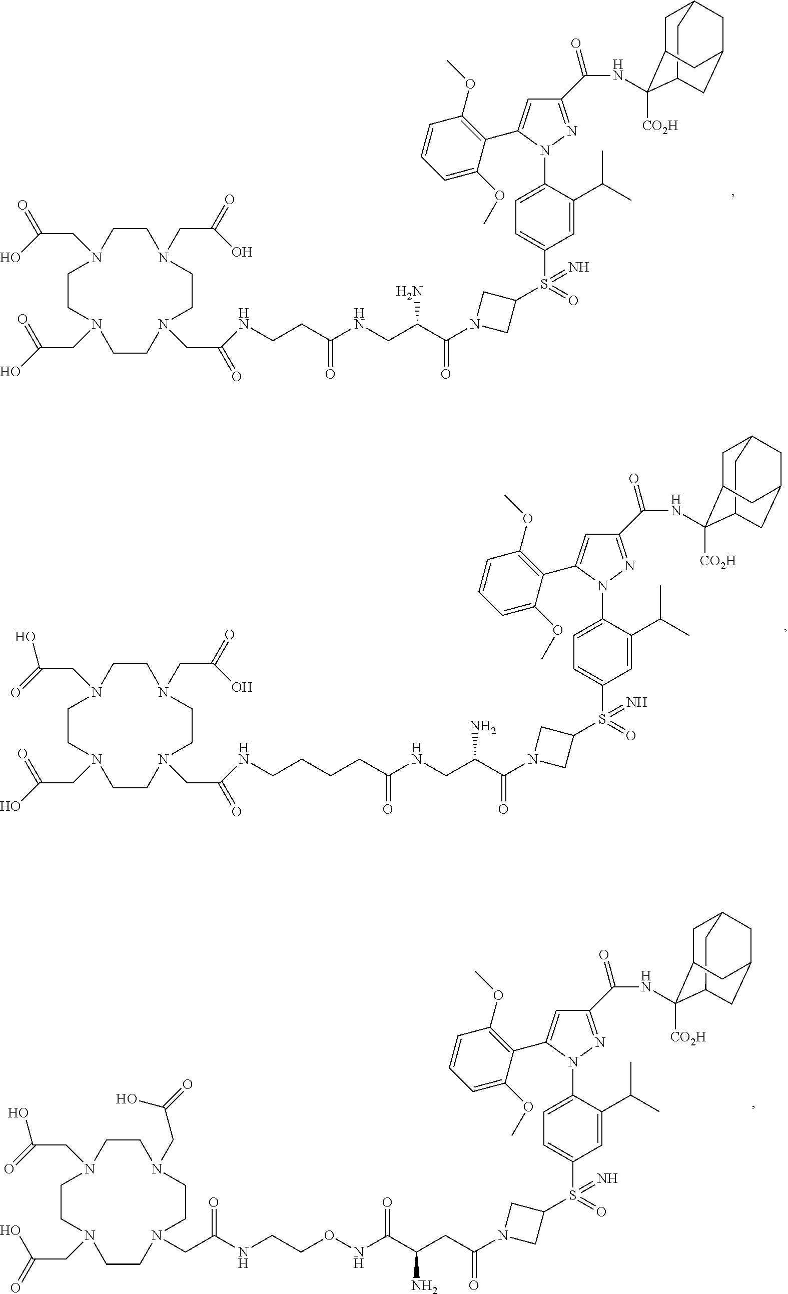 [SMILES: COc1cccc(OC)c1-c1cc(C(=O)NC2(C(=O)O)C3CC4CC(C3)CC2C4)nn1-c1ccc(S(=N)(=O)C2CN(C(=O)[C@@H](N)CNC(=O)CCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(S(=N)(=O)C2CN(C(=O)[C@@H](N)CNC(=O)CCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(S(=N)(=O)C2CN(C(=O)C[C@@H](N)C(=O)NOCCNC(=O)CN3CCN(CC(=O)O)CCN(CC(=O)O)CCN(CC(=O)O)CC3)C2)cc1C(C)C]

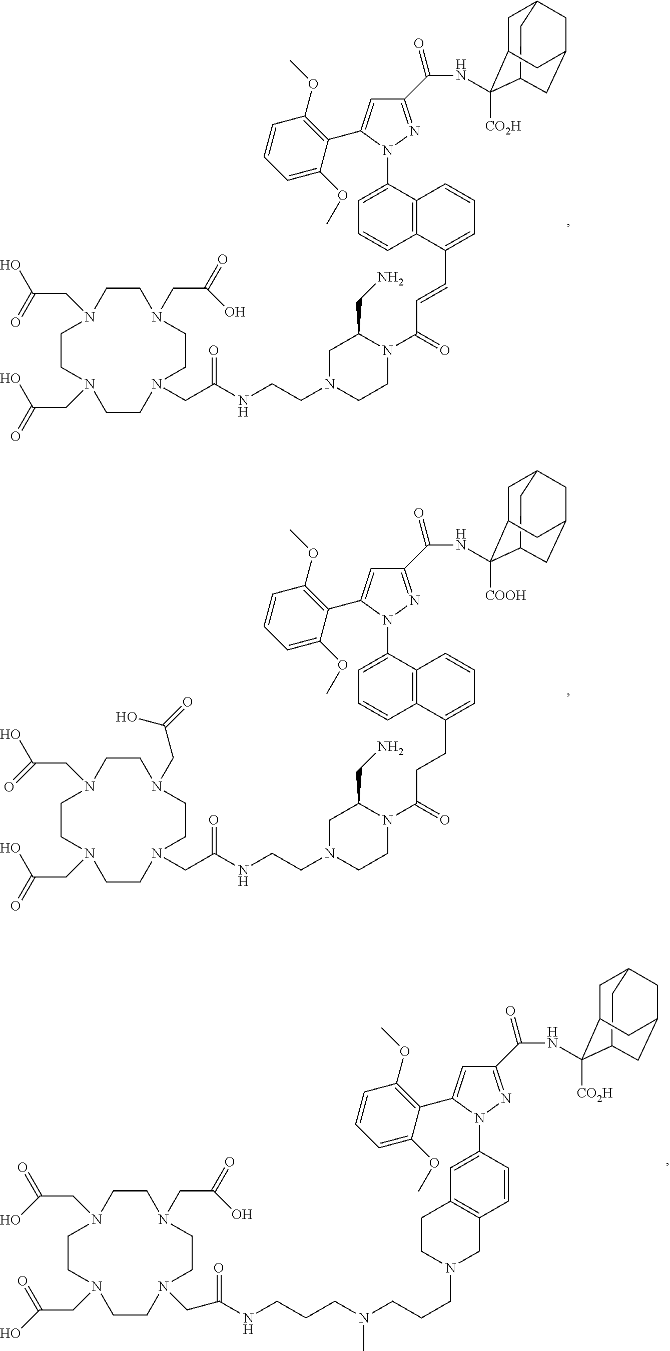 [SMILES: COc1cccc(OC)c1-c1cc(C(=O)NC2(C(=O)O)C3CC4CC(C3)CC2C4)nn1-c1cccc2c(/C=C/C(=O)N3CCN(CCNC(=O)CN4CCN(CC(=O)O)CCN(CC(=O)O)CCN(CC(=O)O)CC4)C[C@H]3CN)cccc12 ; COc1cccc(OC)c1-c1cc(C(=O)NC2(C(=O)O)C3CC4CC(C3)CC2C4)nn1-c1cccc2c(CCC(=O)N3CCN(CCNC(=O)CN4CCN(CC(=O)O)CCN(CC(=O)O)CCN(CC(=O)O)CC4)C[C@H]3CN)cccc12 ; COc1cccc(OC)c1-c1cc(C(=O)NC2(C(=O)O)C3CC4CC(C3)CC2C4)nn1-c1ccc2c(c1)CCN(CCCN(C)CCCNC(=O)CN1CCN(CC(=O)O)CCN(CC(=O)O)CCN(CC(=O)O)CC1)C2]

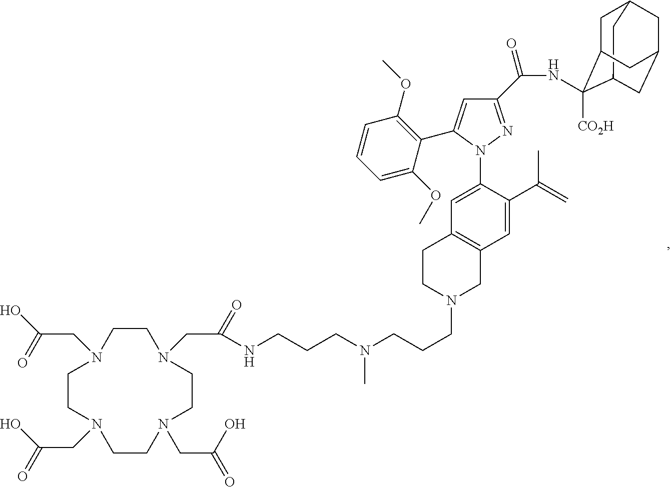 [SMILES: C=C(C)c1cc2c(cc1-n1nc(C(=O)NC3(C(=O)O)C4CC5CC(C4)CC3C5)cc1-c1c(OC)cccc1OC)CCN(CCCN(C)CCCNC(=O)CN1CCN(CC(=O)O)CCN(CC(=O)O)CCN(CC(=O)O)CC1)C2]

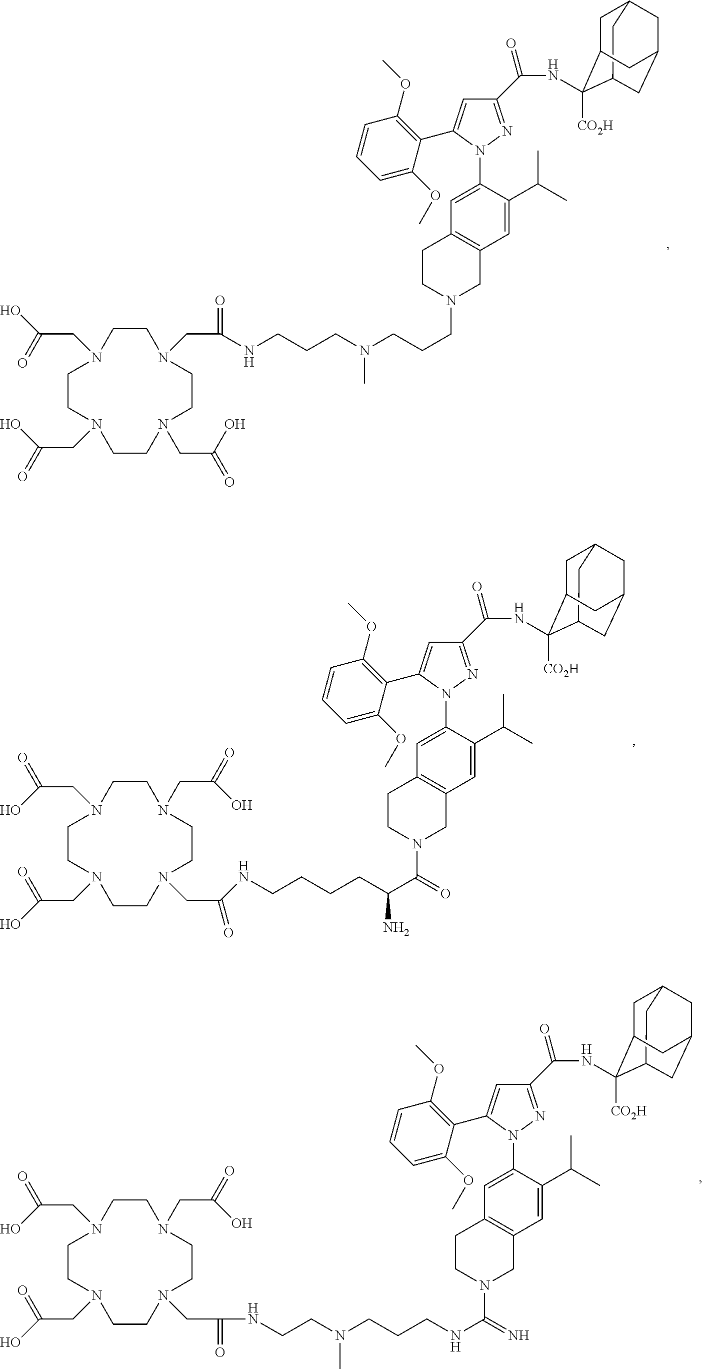 [SMILES: COc1cccc(OC)c1-c1cc(C(=O)NC2(C(=O)O)C3CC4CC(C3)CC2C4)nn1-c1cc2c(cc1C(C)C)CN(CCCN(C)CCCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(C(=O)[C@@H](N)CCCCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(C(=N)NCCCN(C)CCNC(=O)CN1CCN(CC(=O)O)CCN(CC(=O)O)CCN(CC(=O)O)CC1)CC2]

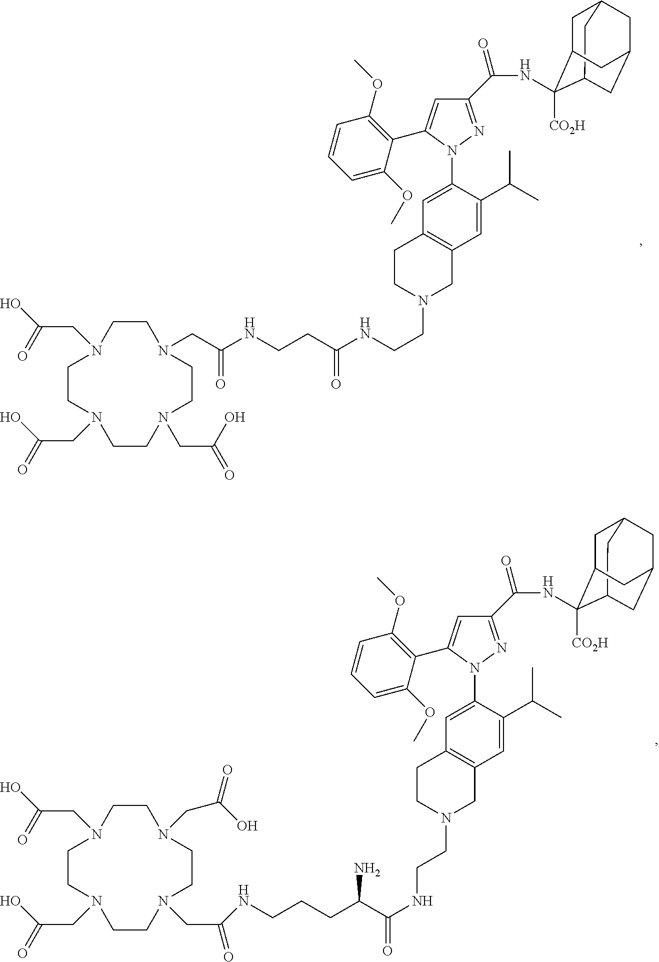 [SMILES: COc1cccc(OC)c1-c1cc(C(=O)NC2(C(=O)O)C3CC4CC(C3)CC2C4)nn1-c1cc2c(cc1C(C)C)CN(CCNC(=O)CCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(CCNC(=O)[C@H](N)CCCNC(=O)CN1CCN(CC(=O)O)CCN(CC(=O)O)CCN(CC(=O)O)CC1)CC2]

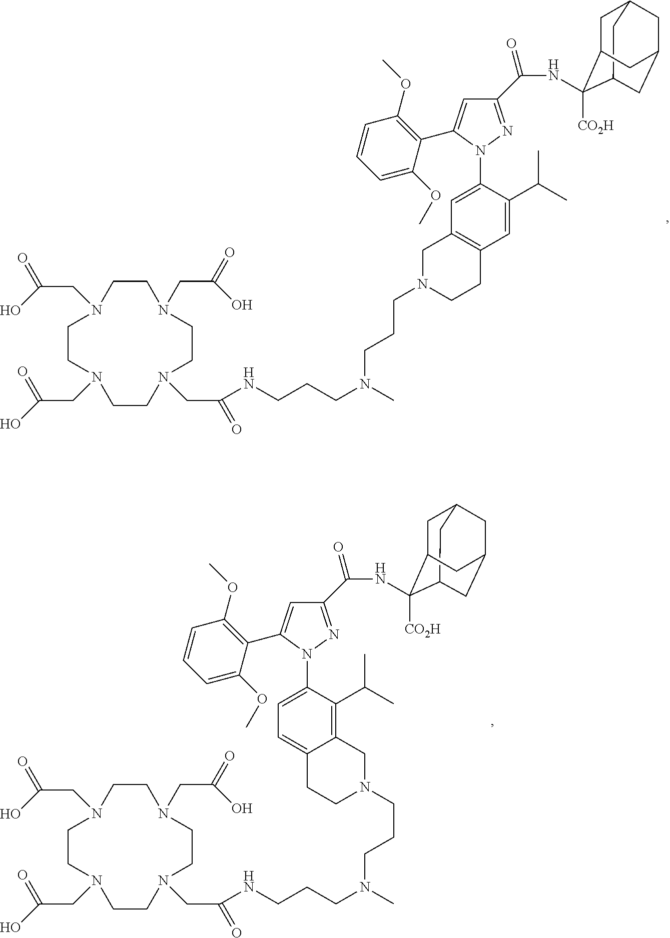 [SMILES: COc1cccc(OC)c1-c1cc(C(=O)NC2(C(=O)O)C3CC4CC(C3)CC2C4)nn1-c1cc2c(cc1C(C)C)CCN(CCCN(C)CCCNC(=O)CN1CCN(CC(=O)O)CCN(CC(=O)O)CCN(CC(=O)O)CC1)C2 ; COc1cccc(OC)c1-c1cc(C(=O)NC2(C(=O)O)C3CC4CC(C3)CC2C4)nn1-c1ccc2c(c1C(C)C)CN(CCCN(C)CCCNC(=O)CN1CCN(CC(=O)O)CCN(CC(=O)O)CCN(CC(=O)O)CC1)CC2]

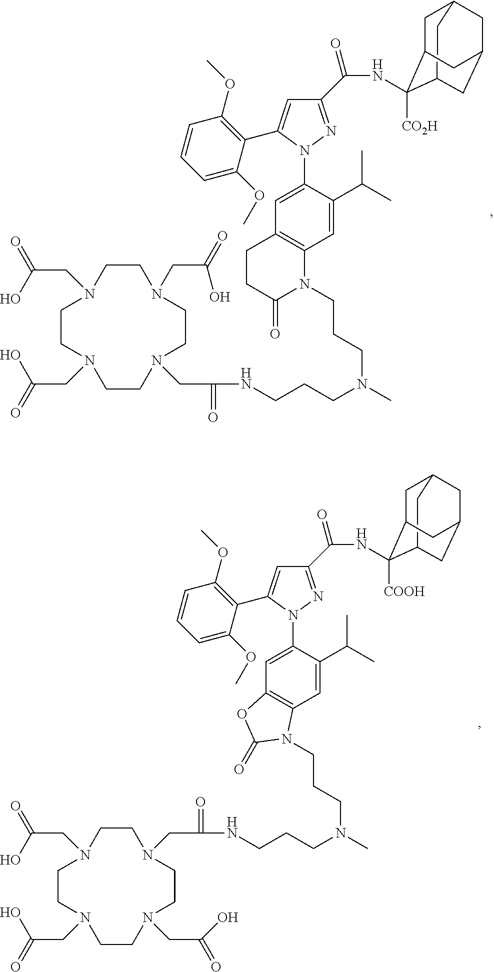 [SMILES: COc1cccc(OC)c1-c1cc(C(=O)NC2(C(=O)O)C3CC4CC(C3)CC2C4)nn1-c1cc2c(cc1C(C)C)N(CCCN(C)CCCNC(=O)CN1CCN(CC(=O)O)CCN(CC(=O)O)CCN(CC(=O)O)CC1)C(=O)CC2 ; COc1cccc(OC)c1-c1cc(C(=O)NC2(C(=O)O)C3CC4CC(C3)CC2C4)nn1-c1cc2oc(=O)n(CCCN(C)CCCNC(=O)CN3CCN(CC(=O)O)CCN(CC(=O)O)CCN(CC(=O)O)CC3)c2cc1C(C)C]

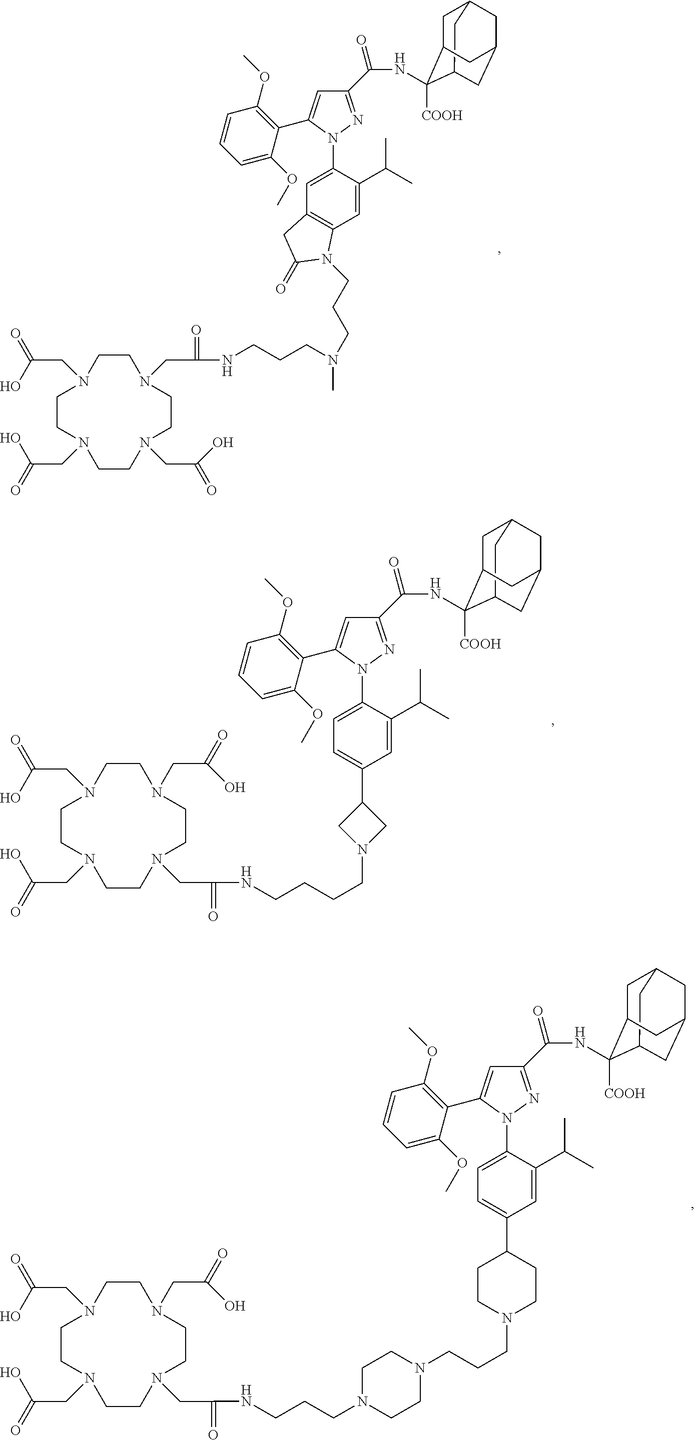 [SMILES: COc1cccc(OC)c1-c1cc(C(=O)NC2(C(=O)O)C3CC4CC(C3)CC2C4)nn1-c1cc2c(cc1C(C)C)N(CCCN(C)CCCNC(=O)CN1CCN(CC(=O)O)CCN(CC(=O)O)CCN(CC(=O)O)CC1)C(=O)C2 ; COc1cccc(OC)c1-c1cc(C(=O)NC2(C(=O)O)C3CC4CC(C3)CC2C4)nn1-c1ccc(C2CN(CCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCN3CCN(CCCNC(=O)CN4CCN(CC(=O)O)CCN(CC(=O)O)CCN(CC(=O)O)CC4)CC3)CC2)cc1C(C)C]

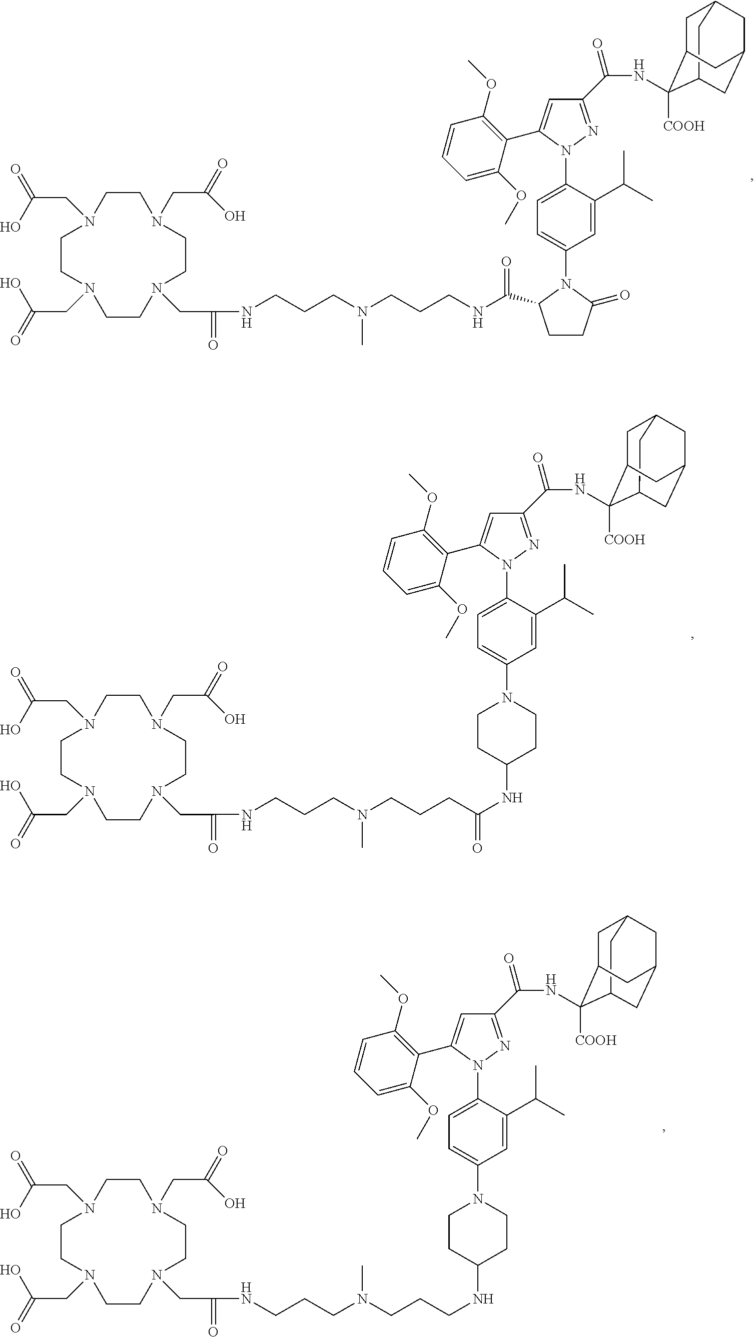 [SMILES: COc1cccc(OC)c1-c1cc(C(=O)NC2(C(=O)O)C3CC4CC(C3)CC2C4)nn1-c1ccc(N2C(=O)CC[C@@H]2C(=O)NCCCN(C)CCCNC(=O)CN2CCN(CC(=O)O)CCN(CC(=O)O)CCN(CC(=O)O)CC2)cc1C(C)C ; COc1cccc(OC)c1-c1cc(C(=O)NC2(C(=O)O)C3CC4CC(C3)CC2C4)nn1-c1ccc(N2CCC(NC(=O)CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCC(NCCCN(C)CCCNC(=O)CN3CCN(CC(=O)O)CCN(CC(=O)O)CCN(CC(=O)O)CC3)CC2)cc1C(C)C]

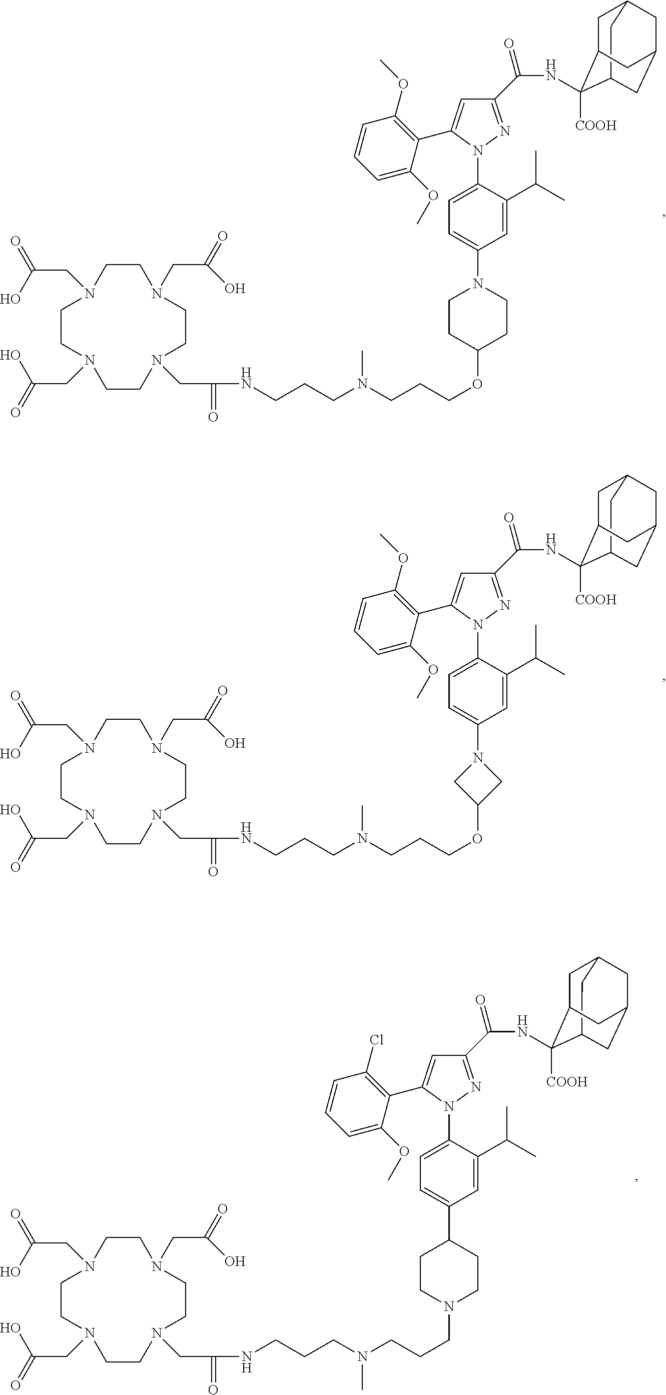 [SMILES: COc1cccc(OC)c1-c1cc(C(=O)NC2(C(=O)O)C3CC4CC(C3)CC2C4)nn1-c1ccc(N2CCC(OCCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CC(OCCCN(C)CCCNC(=O)CN3CCN(CC(=O)O)CCN(CC(=O)O)CCN(CC(=O)O)CC3)C2)cc1C(C)C ; COc1cccc(Cl)c1-c1cc(C(=O)NC2(C(=O)O)C3CC4CC(C3)CC2C4)nn1-c1ccc(C2CCN(CCCN(C)CCCNC(=O)CN3CCN(CC(=O)O)CCN(CC(=O)O)CCN(CC(=O)O)CC3)CC2)cc1C(C)C]

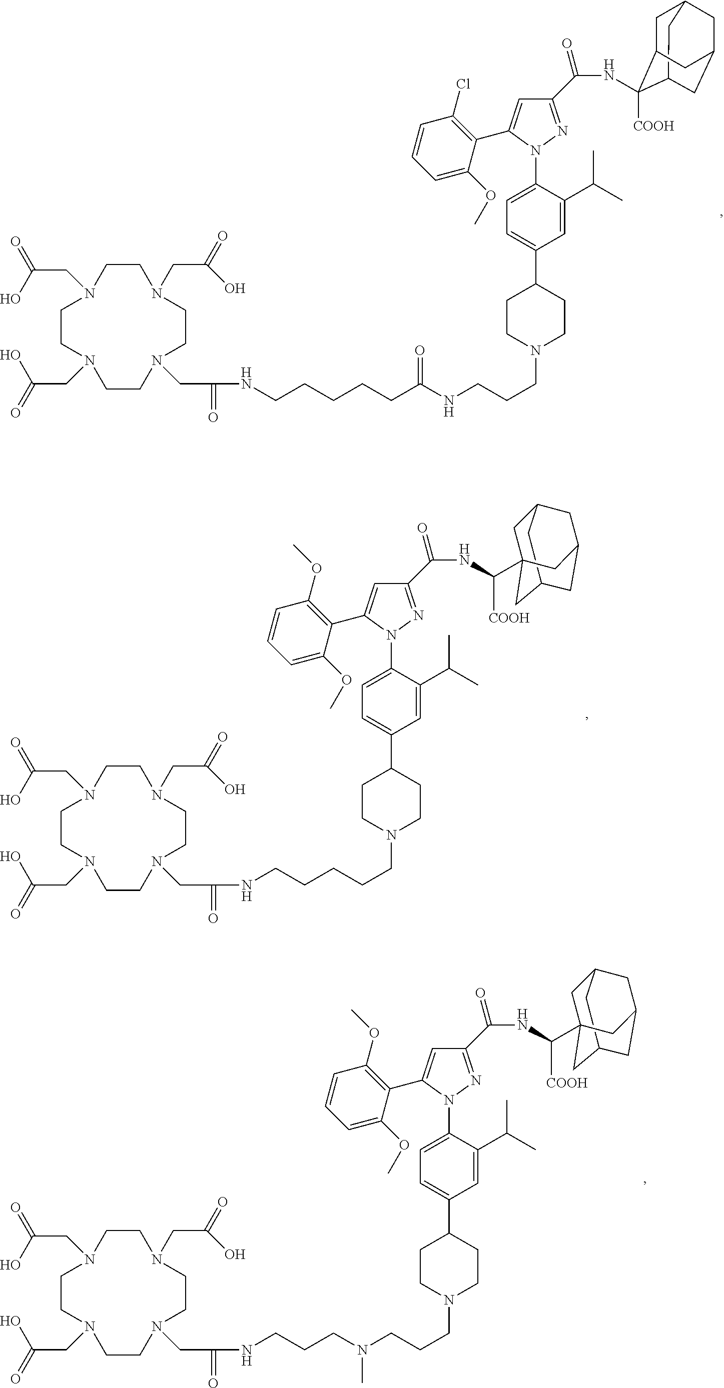 [SMILES: COc1cccc(Cl)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2CCN(CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2CCN(CCCN(C)CCCNC(=O)CN3CCN(CC(=O)O)CCN(CC(=O)O)CCN(CC(=O)O)CC3)CC2)cc1C(C)C]

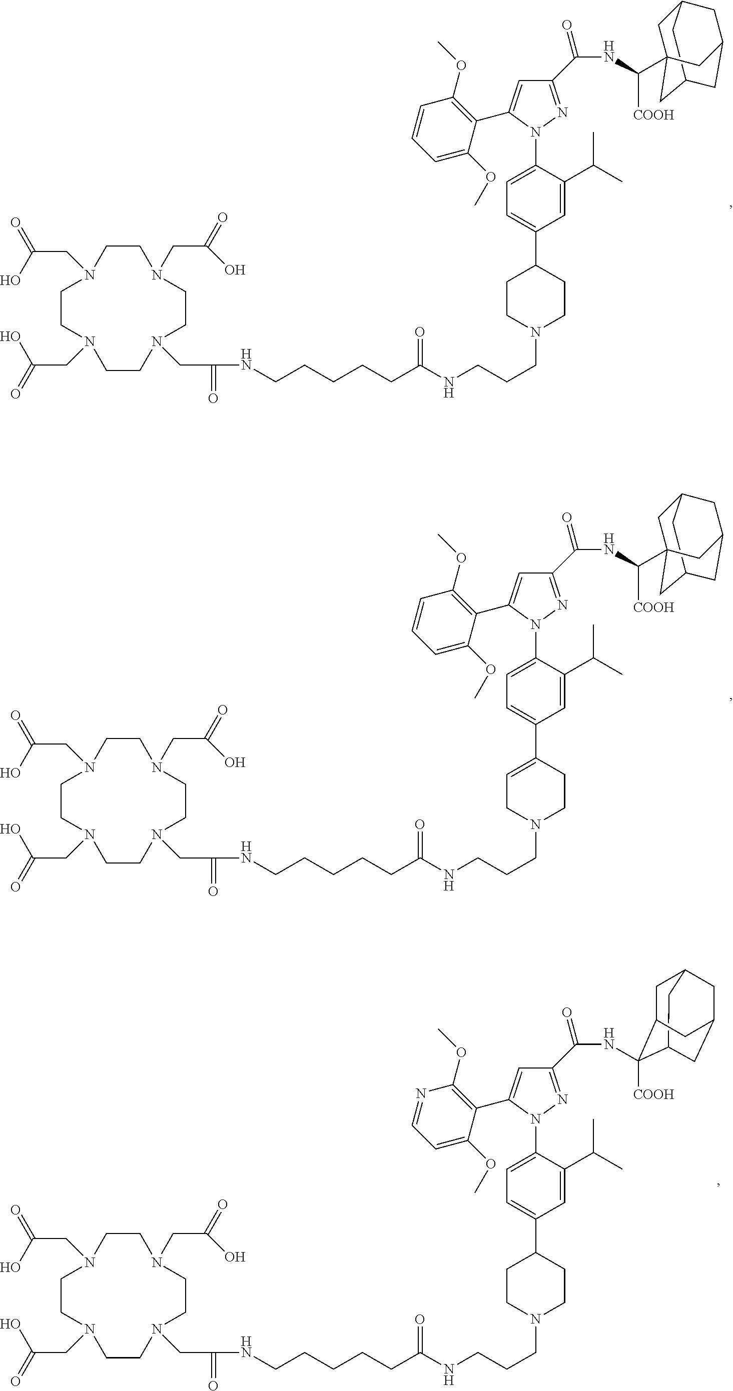 [SMILES: COc1cccc(OC)c1-c1cc(C(=O)N[C@H](C(=O)O)C23CC4CC(CC(C4)C2)C3)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2=CCN(CCCNC(=O)CCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C]

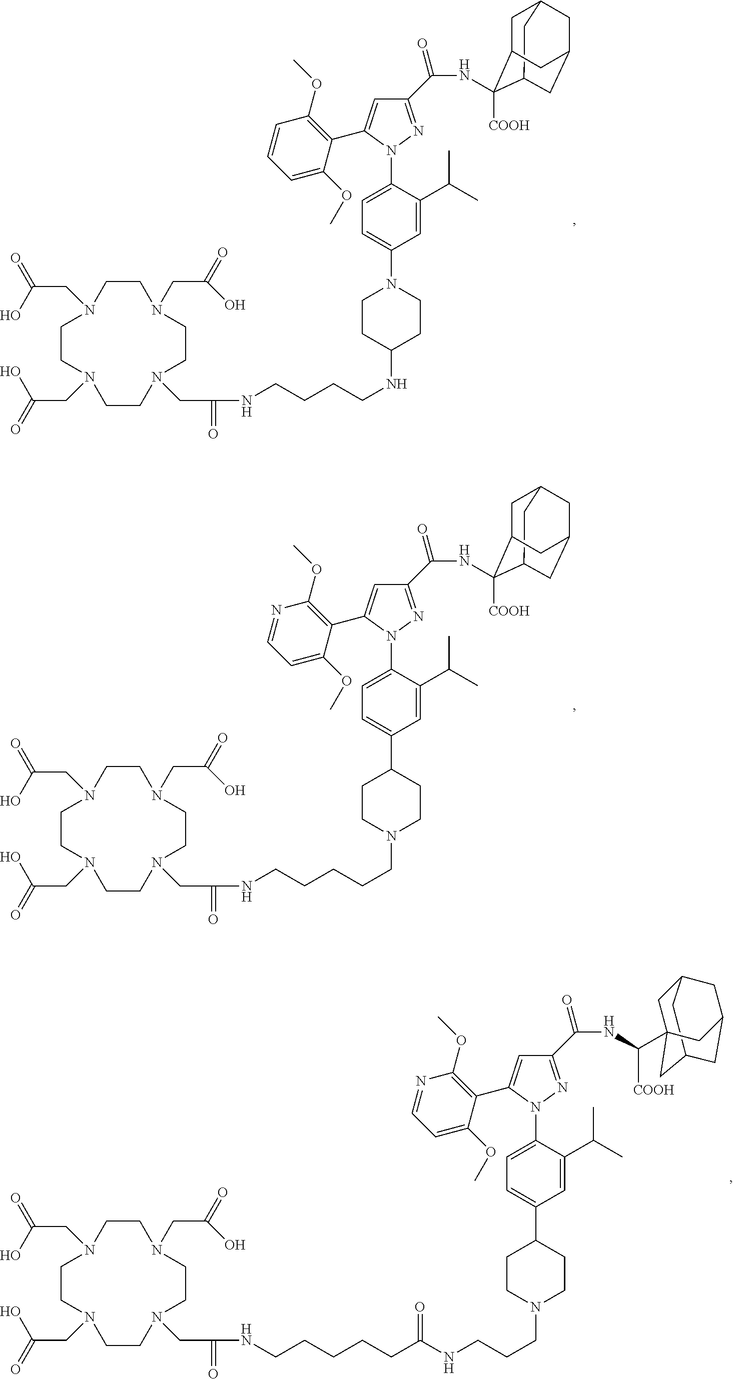 [SMILES: COc1cccc(OC)c1-c1cc(C(=O)NC2(C(=O)O)C3CC4CC(C3)CC2C4)nn1-c1ccc(N2CCC(NCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)N[C@H](C(=O)O)C23CC4CC(CC(C4)C2)C3)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C]

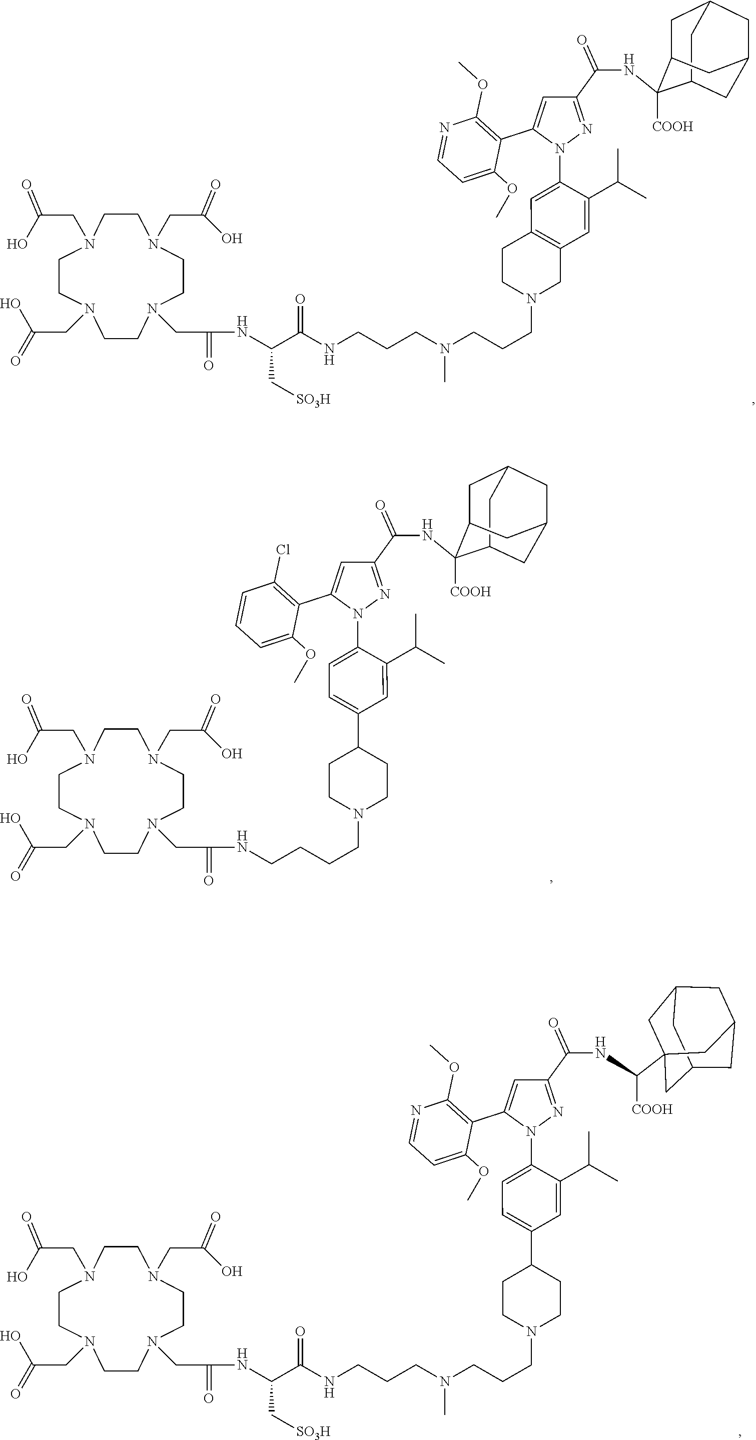

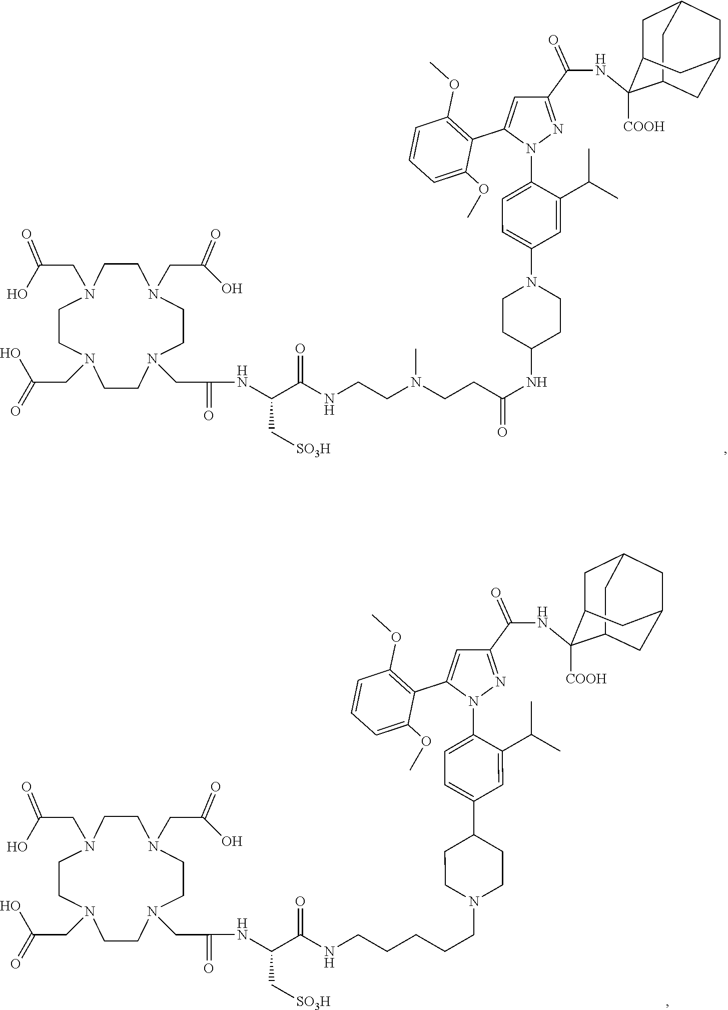

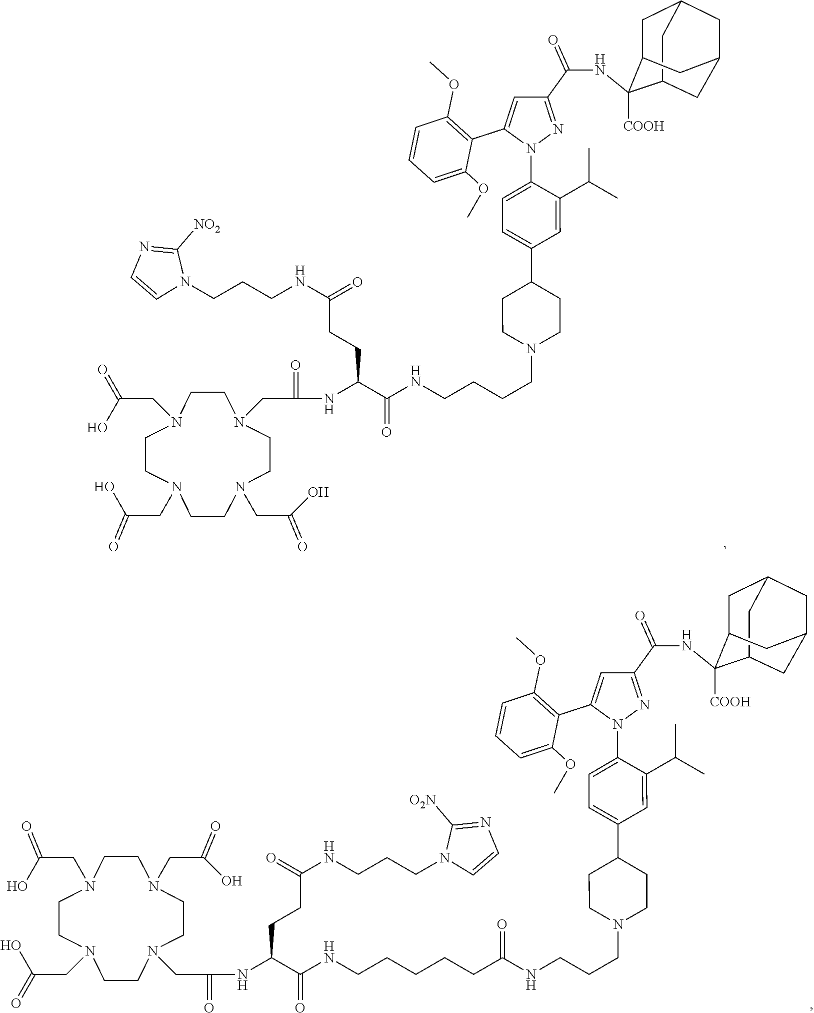

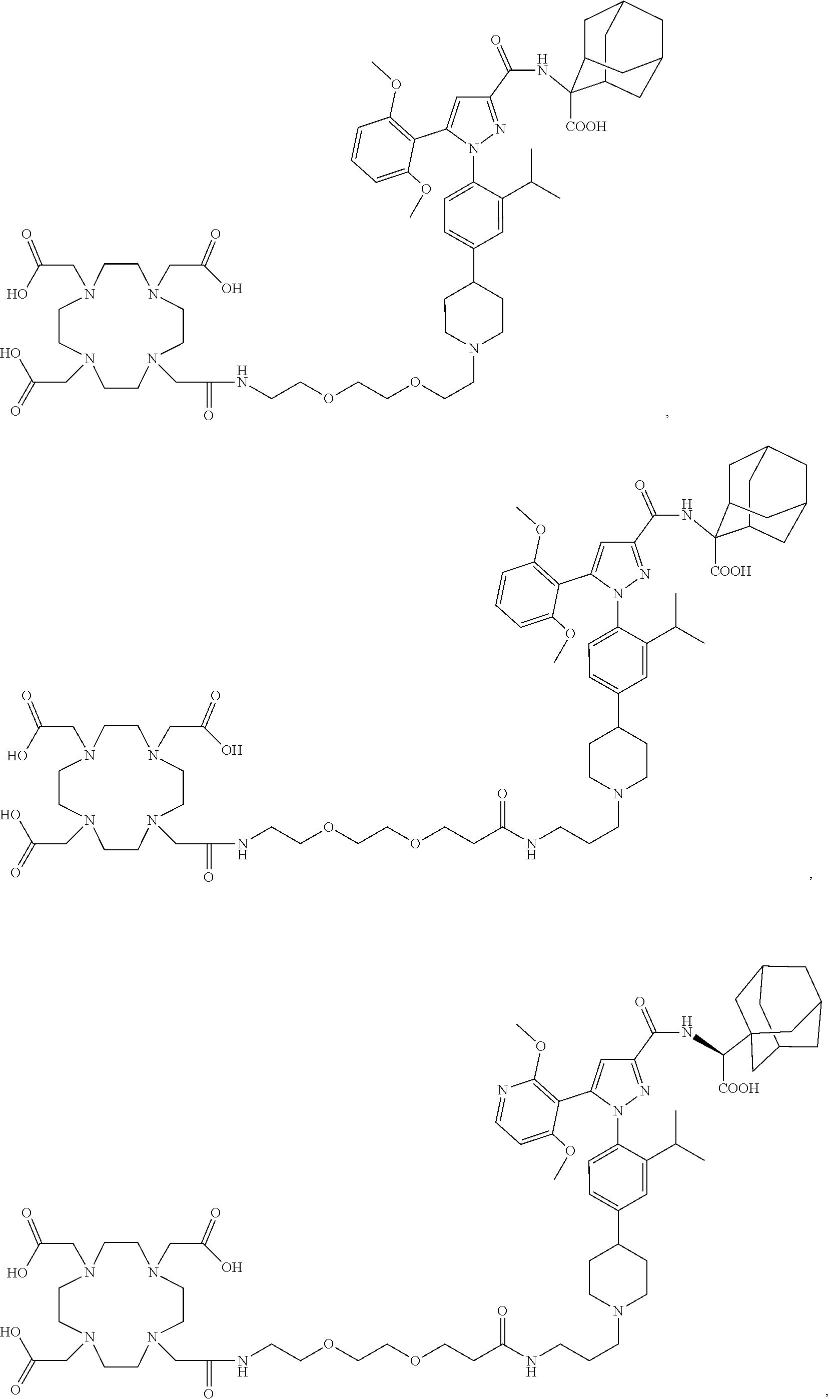

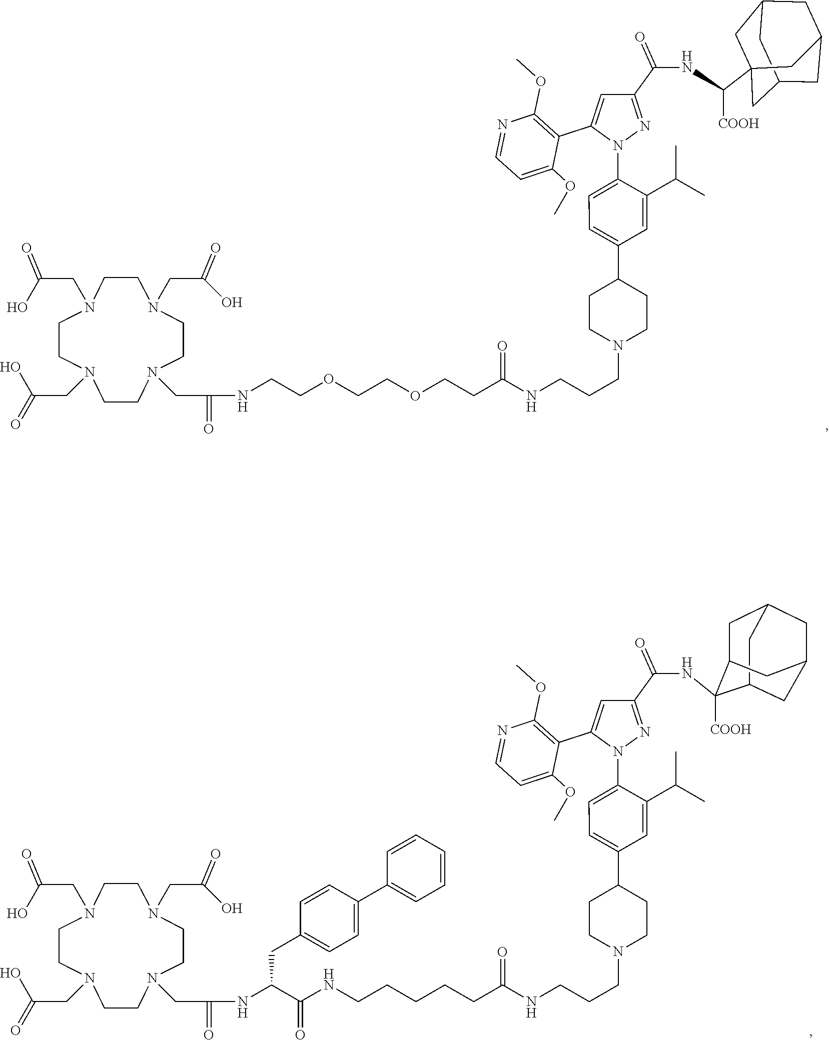

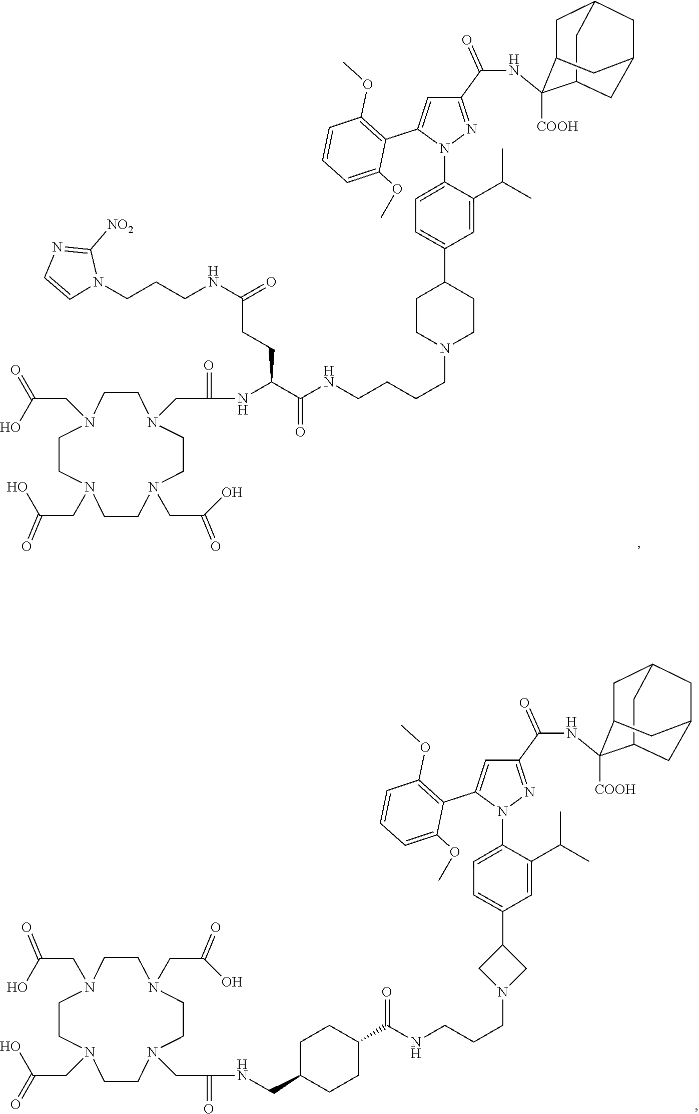

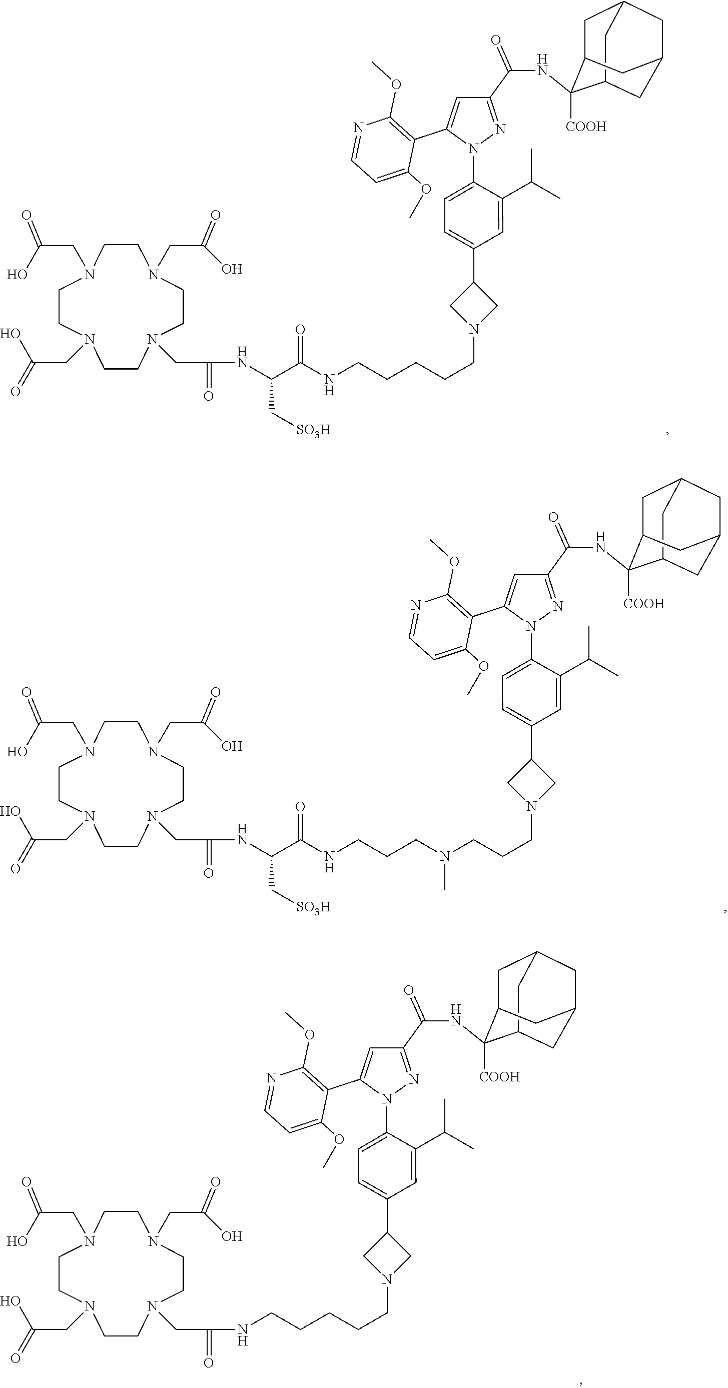

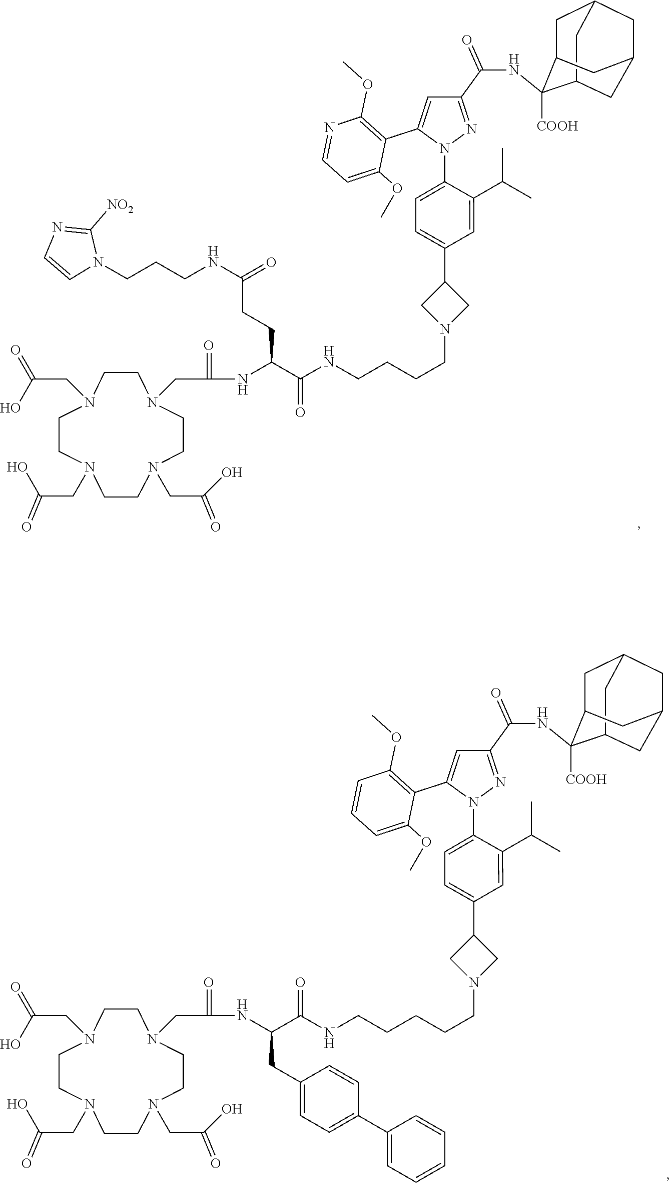

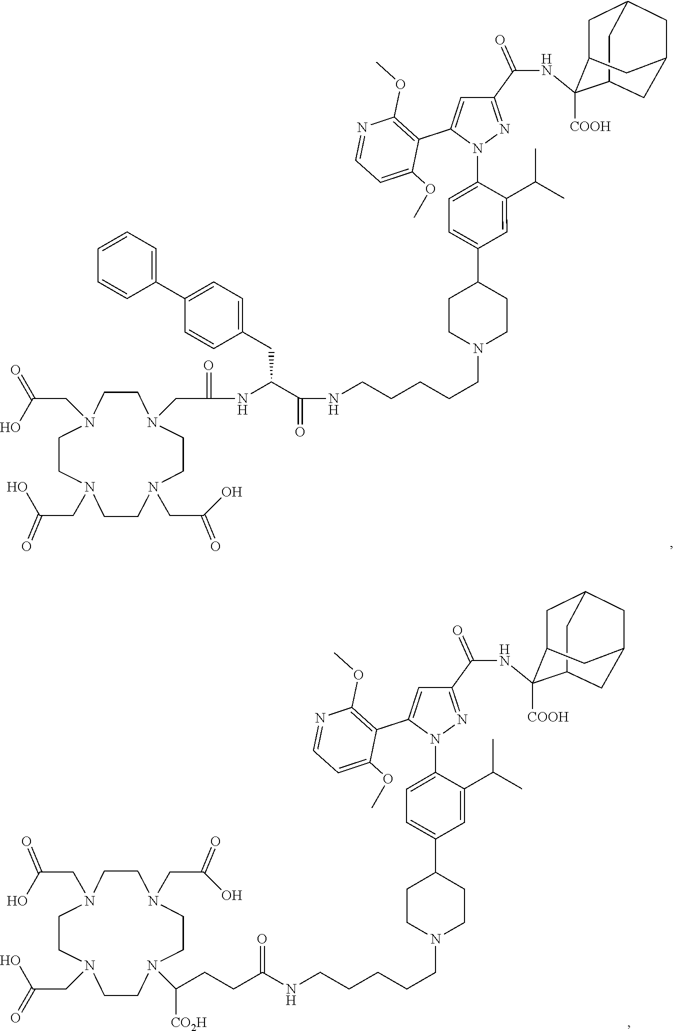

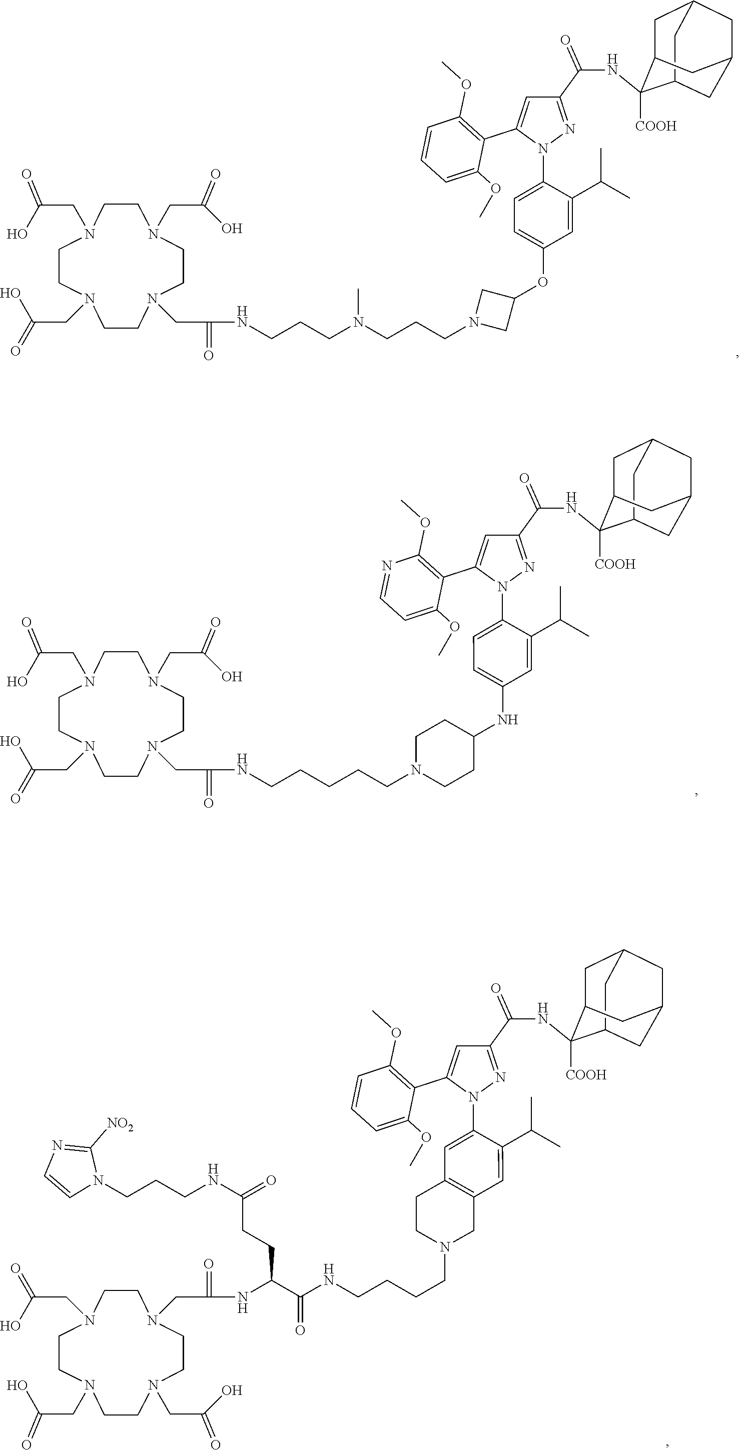

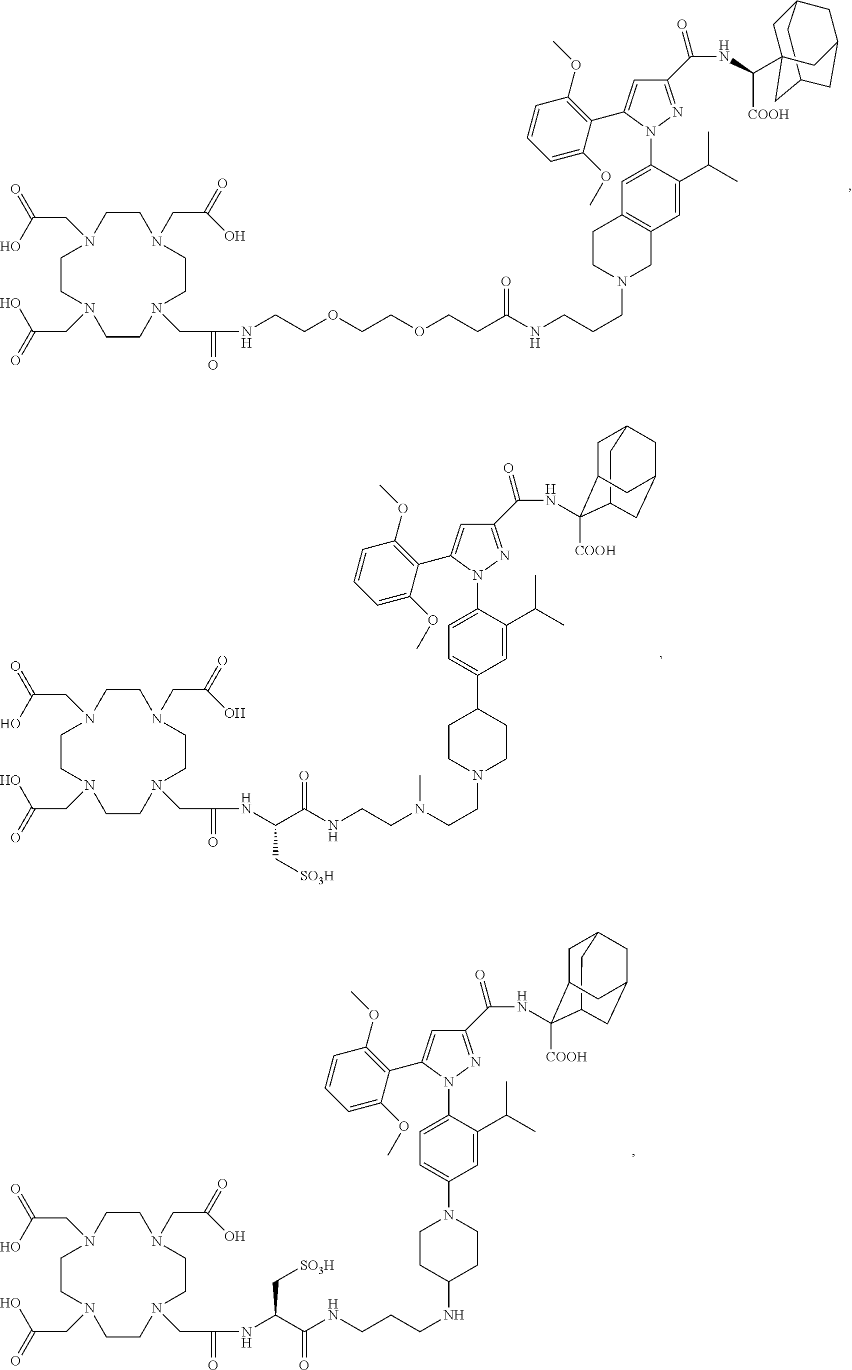 [SMILES: COc1cccc(OC)c1-c1cc(C(=O)N[C@H](C(=O)O)C23CC4CC(CC(C4)C2)C3)nn1-c1cc2c(cc1C(C)C)CN(CCCNC(=O)CCOCCOCCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1ccc(C2CCN(CCN(C)CCNC(=O)[C@H](CS(=O)(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCC(NCCCNC(=O)[C@H](CS(=O)(=O)O)NC(=O)CN3CCN(CC(=O)O)CCN(CC(=O)O)CCN(CC(=O)O)CC3)CC2)cc1C(C)C]

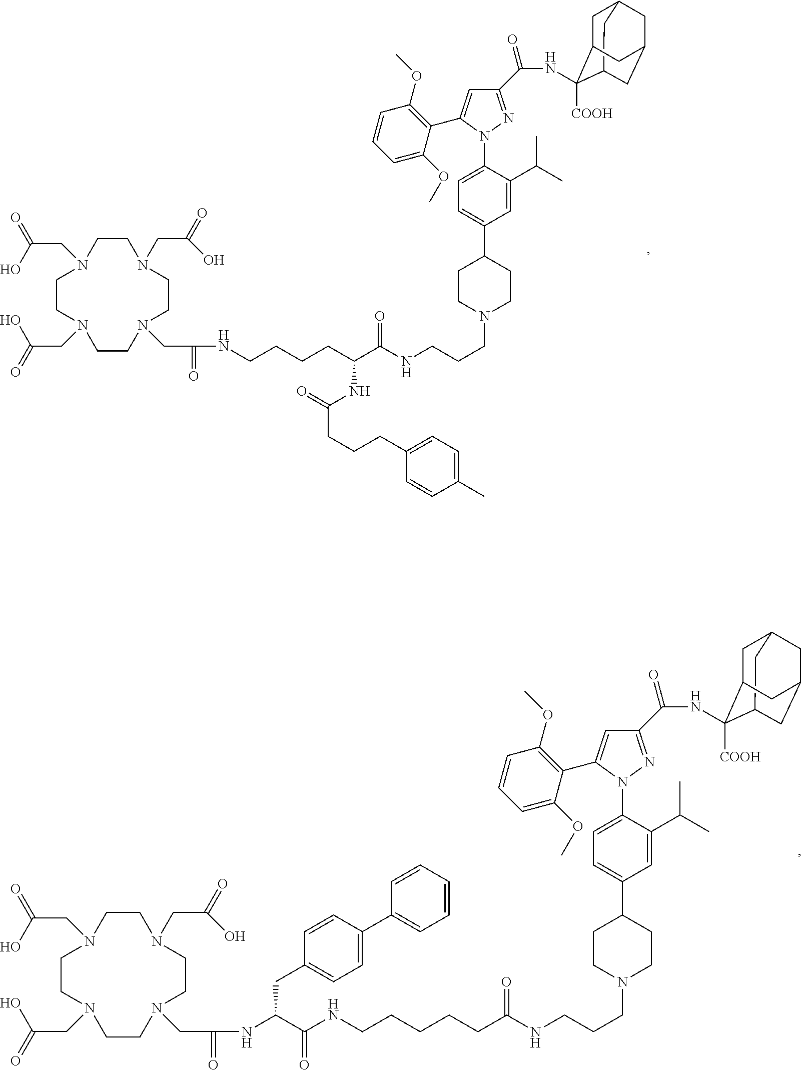 [SMILES: COc1cccc(OC)c1-c1cc(C(=O)NC2(C(=O)O)C3CC4CC(C3)CC2C4)nn1-c1ccc(C2CCN(CCCNC(=O)[C@@H](CCCCNC(=O)CN3CCN(CC(=O)O)CCN(CC(=O)O)CCN(CC(=O)O)CC3)NC(=O)CCCc3ccc(C)cc3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CCCCCNC(=O)[C@@H](Cc3ccc(-c4ccccc4)cc3)NC(=O)CN3CCN(CC(=O)O)CCN(CC(=O)O)CCN(CC(=O)O)CC3)CC2)cc1C(C)C]

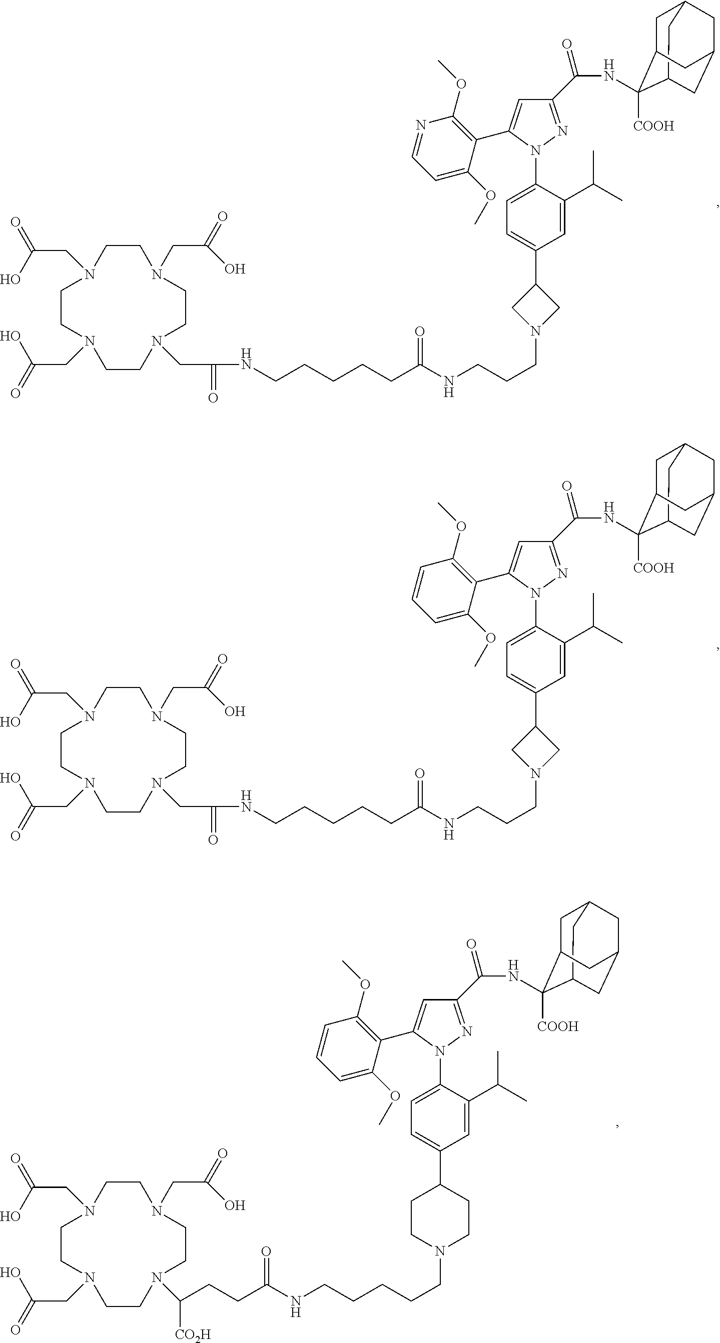 [SMILES: COc1ccnc(OC)c1-c1cc(C(=O)NC2(C(=O)O)C3CC4CC(C3)CC2C4)nn1-c1ccc(C2CN(CCCNC(=O)CCCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CN(CCCNC(=O)CCCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCCCNC(=O)CCC(C(=O)O)N3CCN(CC(=O)O)CCN(CC(=O)O)CCN(CC(=O)O)CC3)CC2)cc1C(C)C]

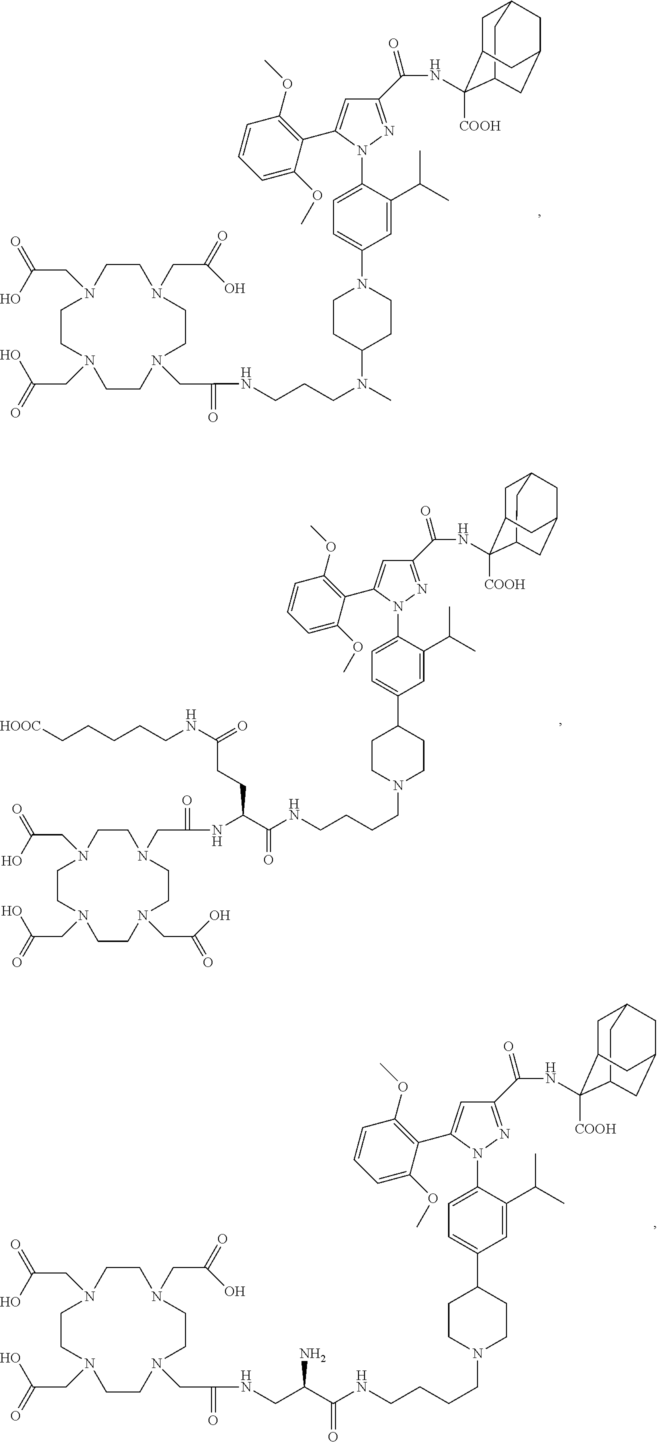 [SMILES: COc1cccc(OC)c1-c1cc(C(=O)NC2(C(=O)O)C3CC4CC(C3)CC2C4)nn1-c1ccc(N2CCC(N(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCC(=O)NCCCCCC(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](N)CNC(=O)CN3CCN(CC(=O)O)CCN(CC(=O)O)CCN(CC(=O)O)CC3)CC2)cc1C(C)C]

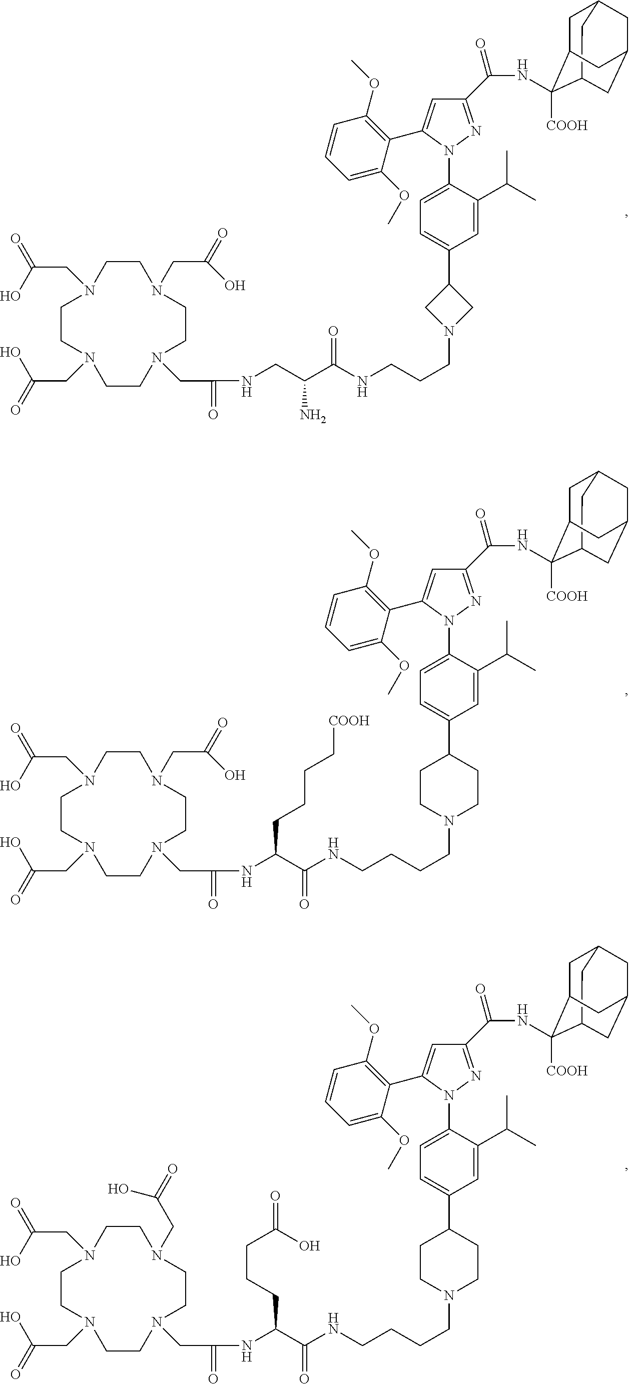 [SMILES: COc1cccc(OC)c1-c1cc(C(=O)NC2(C(=O)O)C3CC4CC(C3)CC2C4)nn1-c1ccc(C2CN(CCCNC(=O)[C@H](N)CNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCCCC(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCCC(=O)O)NC(=O)CN3CCN(CC(=O)O)CCN(CC(=O)O)CCN(CC(=O)O)CC3)CC2)cc1C(C)C]

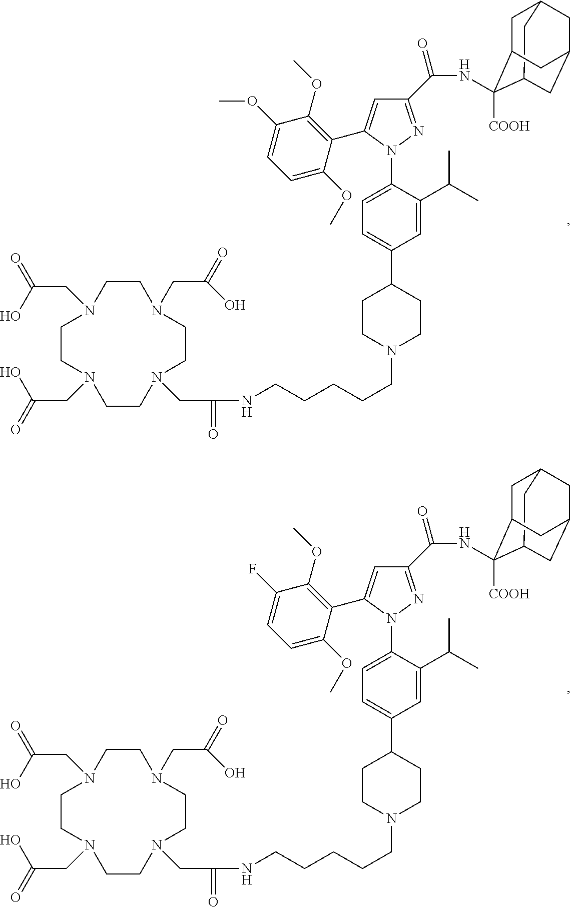 [SMILES: COc1ccc(OC)c(-c2cc(C(=O)NC3(C(=O)O)C4CC5CC(C4)CC3C5)nn2-c2ccc(C3CCN(CCCCCNC(=O)CN4CCN(CC(=O)O)CCN(CC(=O)O)CCN(CC(=O)O)CC4)CC3)cc2C(C)C)c1OC ; COc1ccc(F)c(OC)c1-c1cc(C(=O)NC2(C(=O)O)C3CC4CC(C3)CC2C4)nn1-c1ccc(C2CCN(CCCCCNC(=O)CN3CCN(CC(=O)O)CCN(CC(=O)O)CCN(CC(=O)O)CC3)CC2)cc1C(C)C]

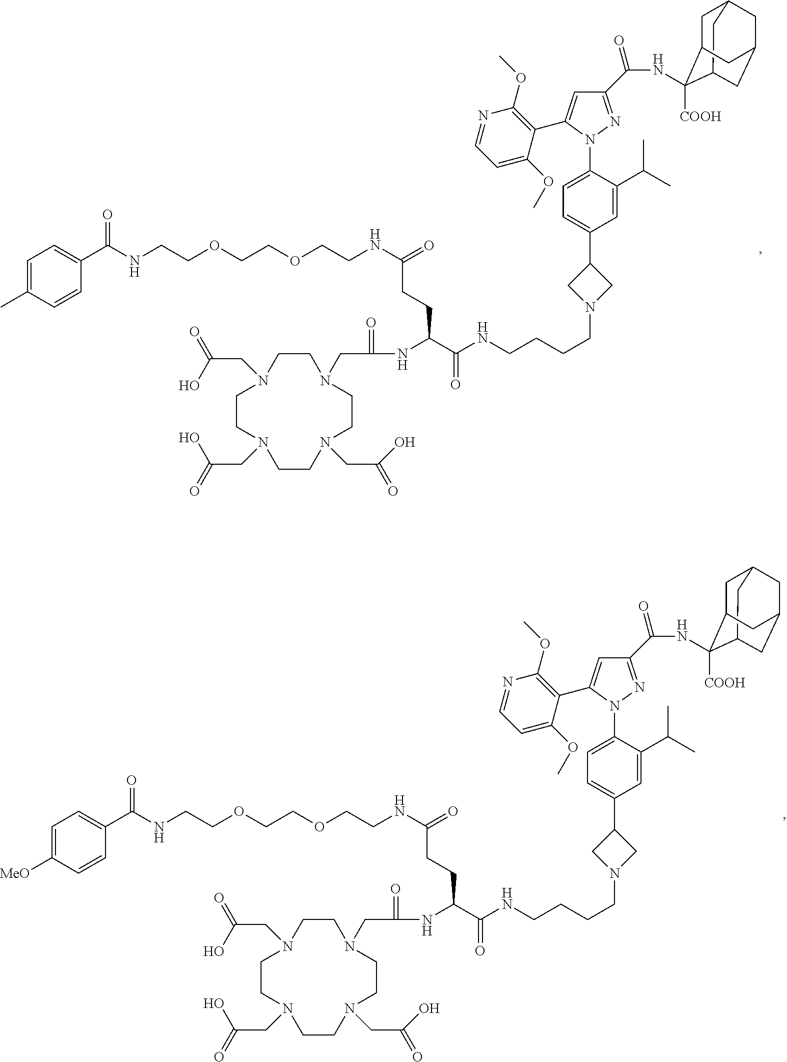 [SMILES: COc1ccnc(OC)c1-c1cc(C(=O)NC2(C(=O)O)C3CC4CC(C3)CC2C4)nn1-c1ccc(C2CN(CCCCNC(=O)[C@H](CCC(=O)NCCOCCOCCNC(=O)c3ccc(C)cc3)NC(=O)CN3CCN(CC(=O)O)CCN(CC(=O)O)CCN(CC(=O)O)CC3)C2)cc1C(C)C ; COc1ccc(C(=O)NCCOCCOCCNC(=O)CC[C@H](NC(=O)CN2CCN(CC(=O)O)CCN(CC(=O)O)CCN(CC(=O)O)CC2)C(=O)NCCCCN2CC(c3ccc(-n4nc(C(=O)NC5(C(=O)O)C6CC7CC(C6)CC5C7)cc4-c4c(OC)ccnc4OC)c(C(C)C)c3)C2)cc1]

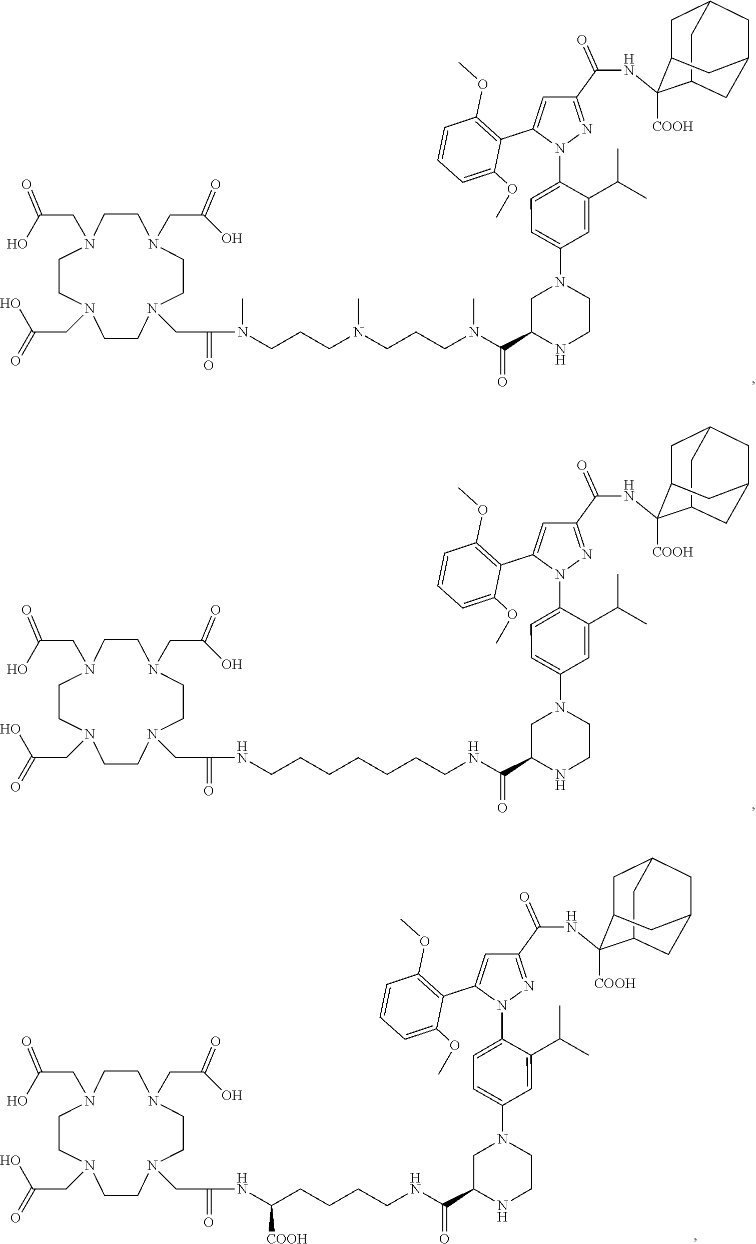

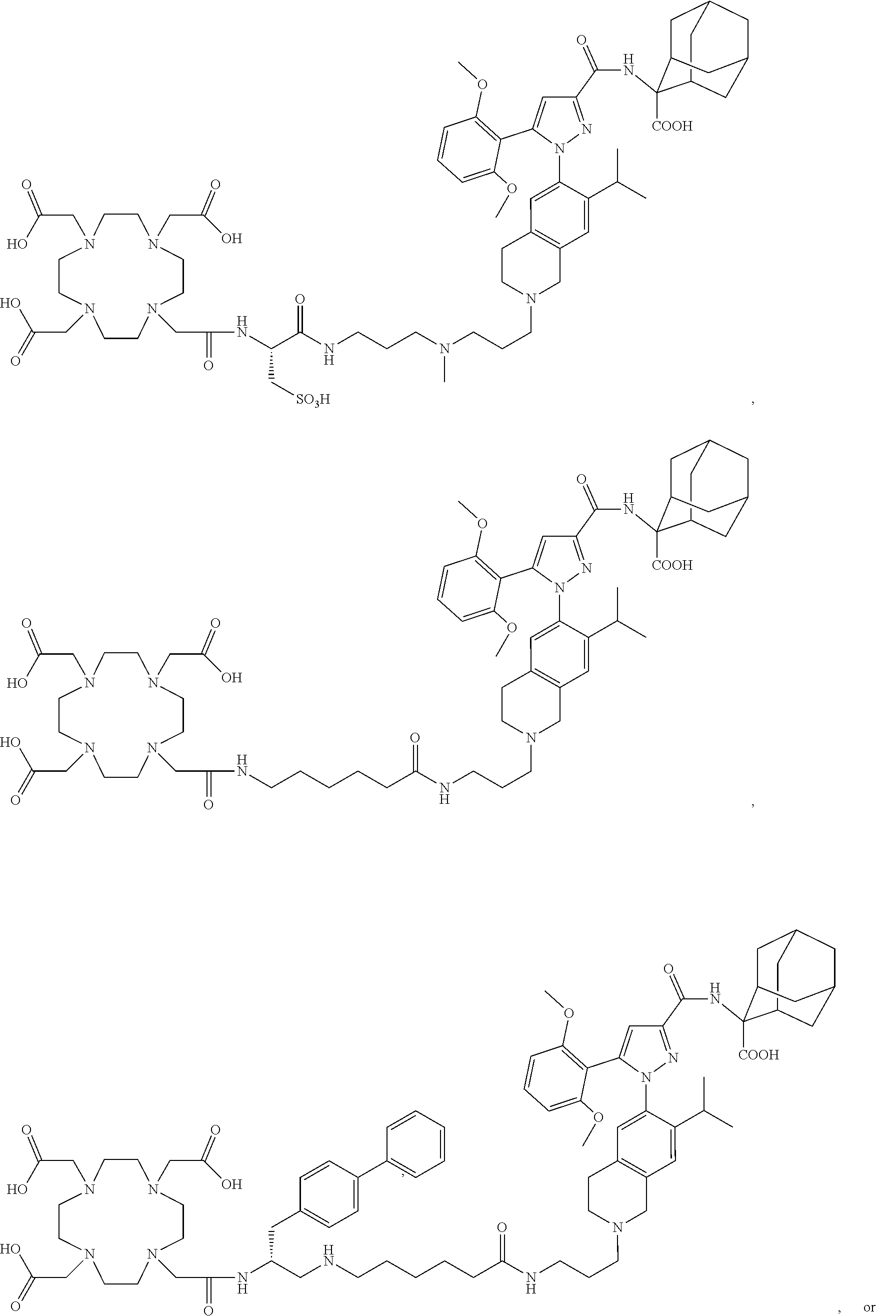

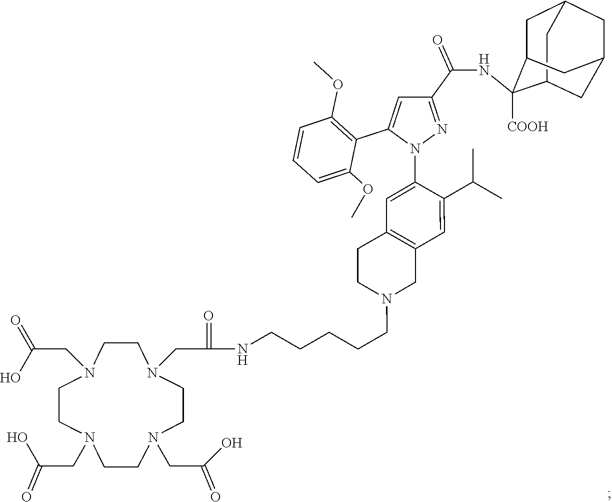

or a radionuclide complex thereof.

In some embodiments, the compound of Formula (A) or Formula (I) is:

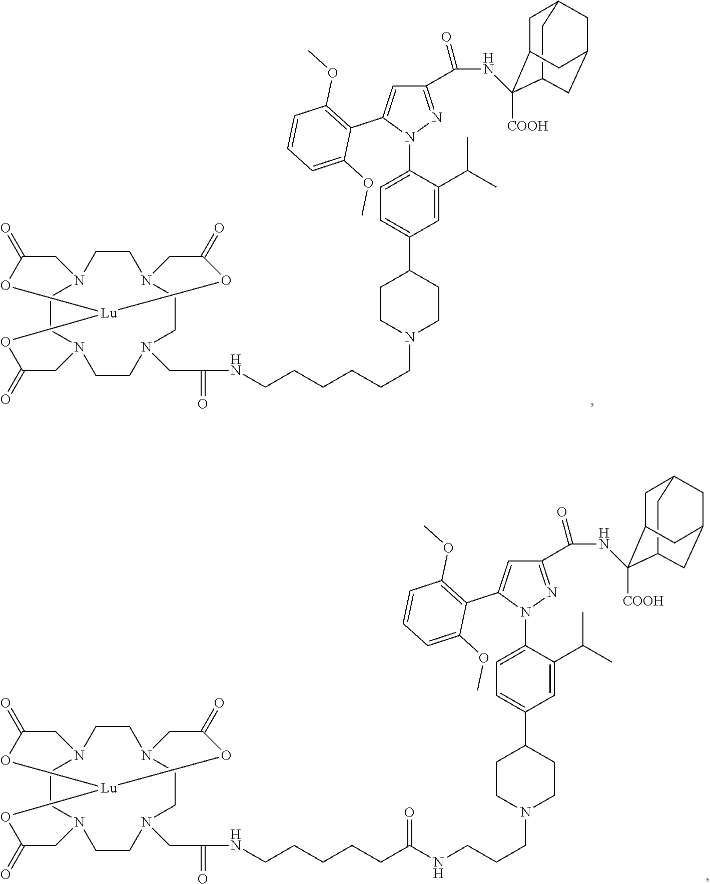

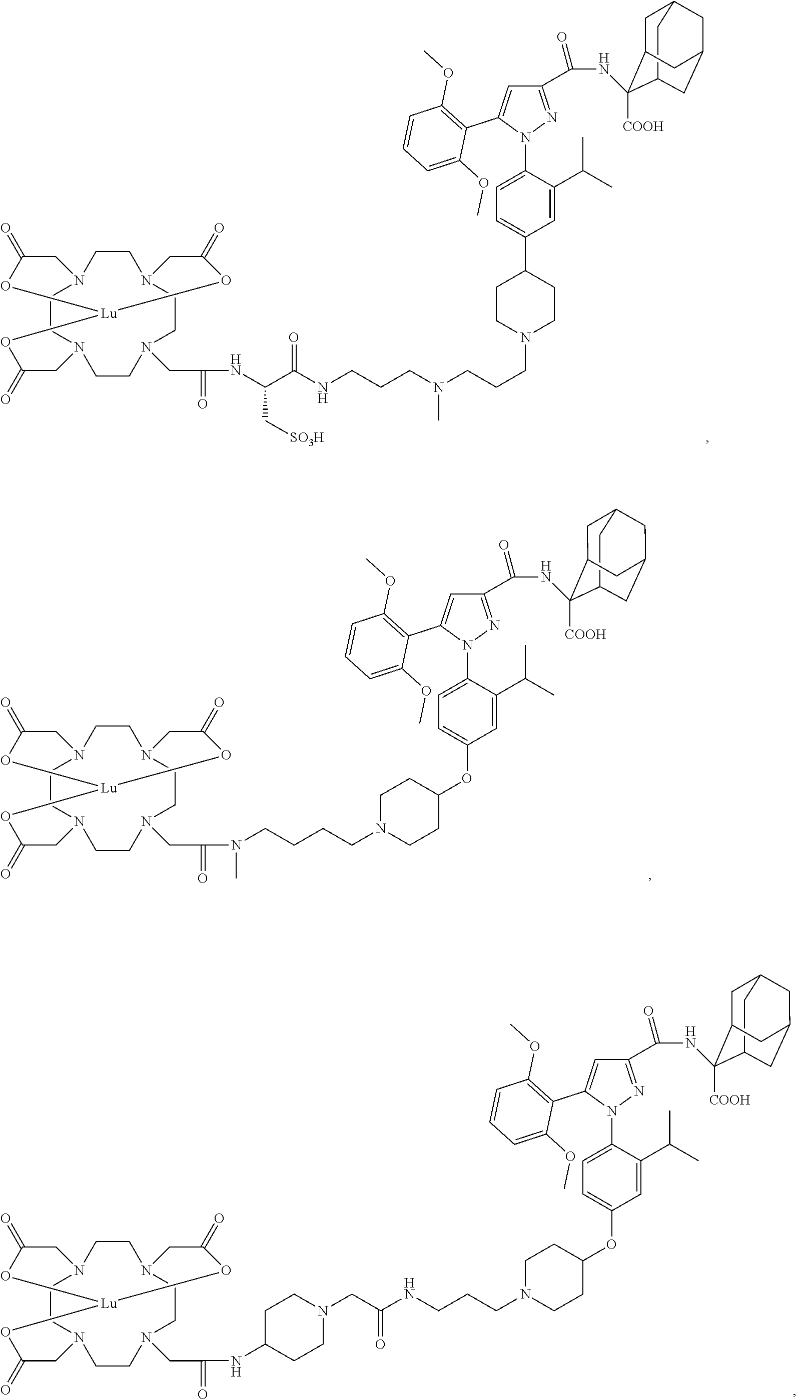

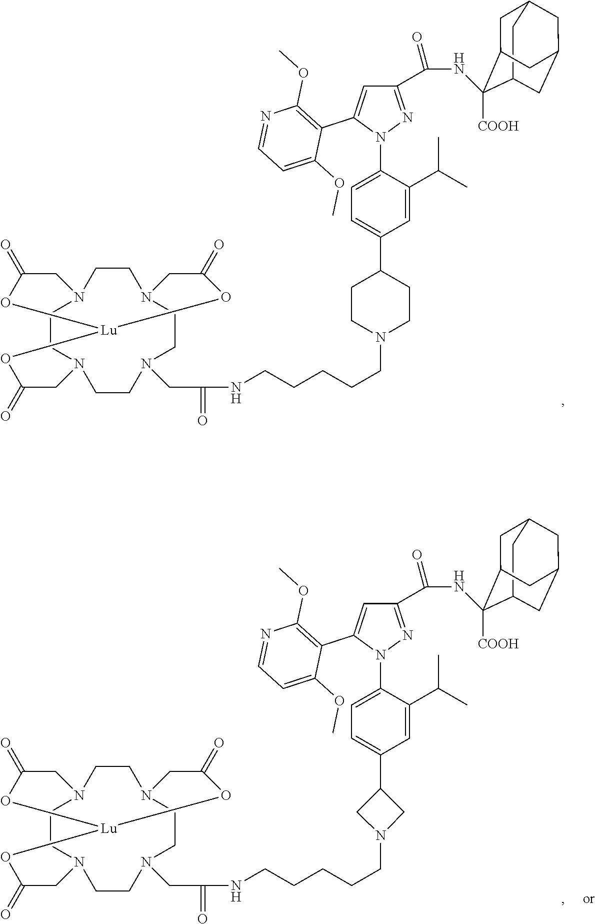

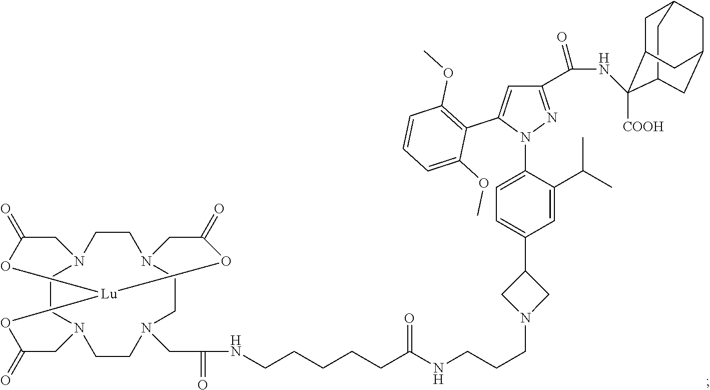

or a pharmaceutically acceptable salt thereof.

In some embodiments, the compound of Formula (A) or Formula (I) is:

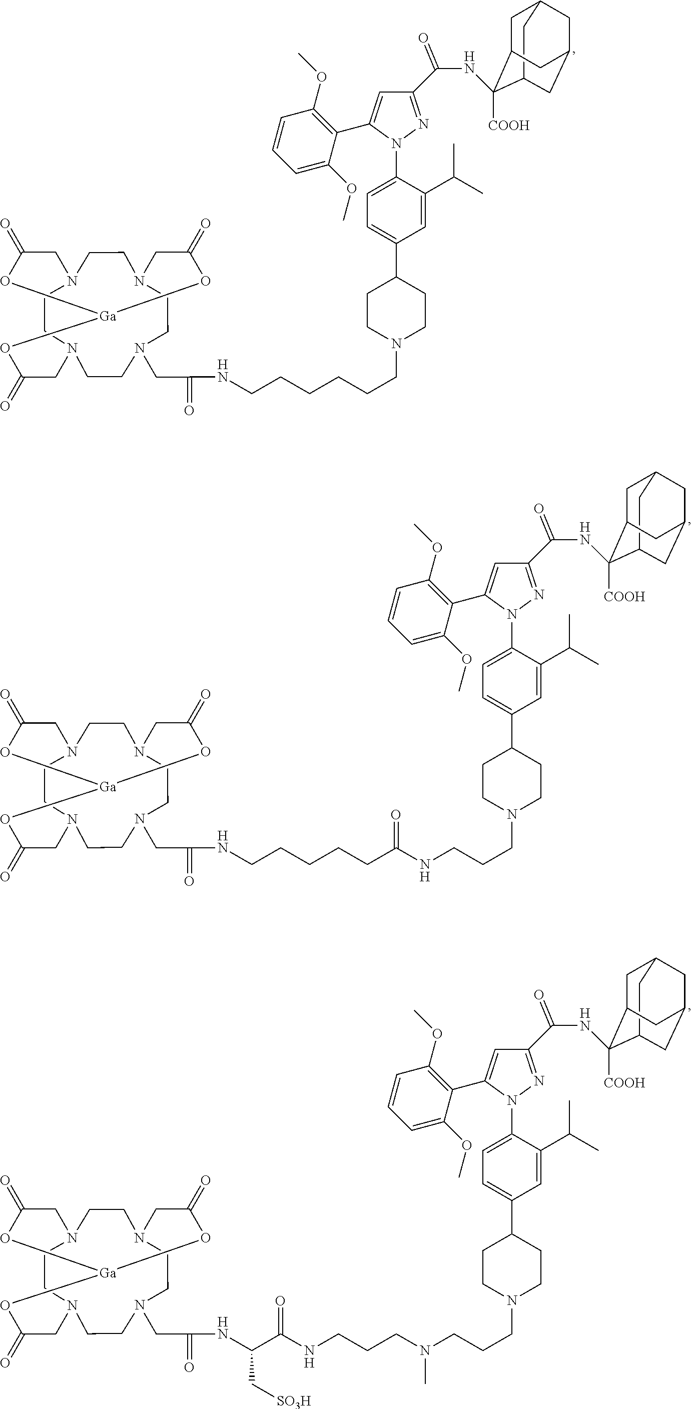

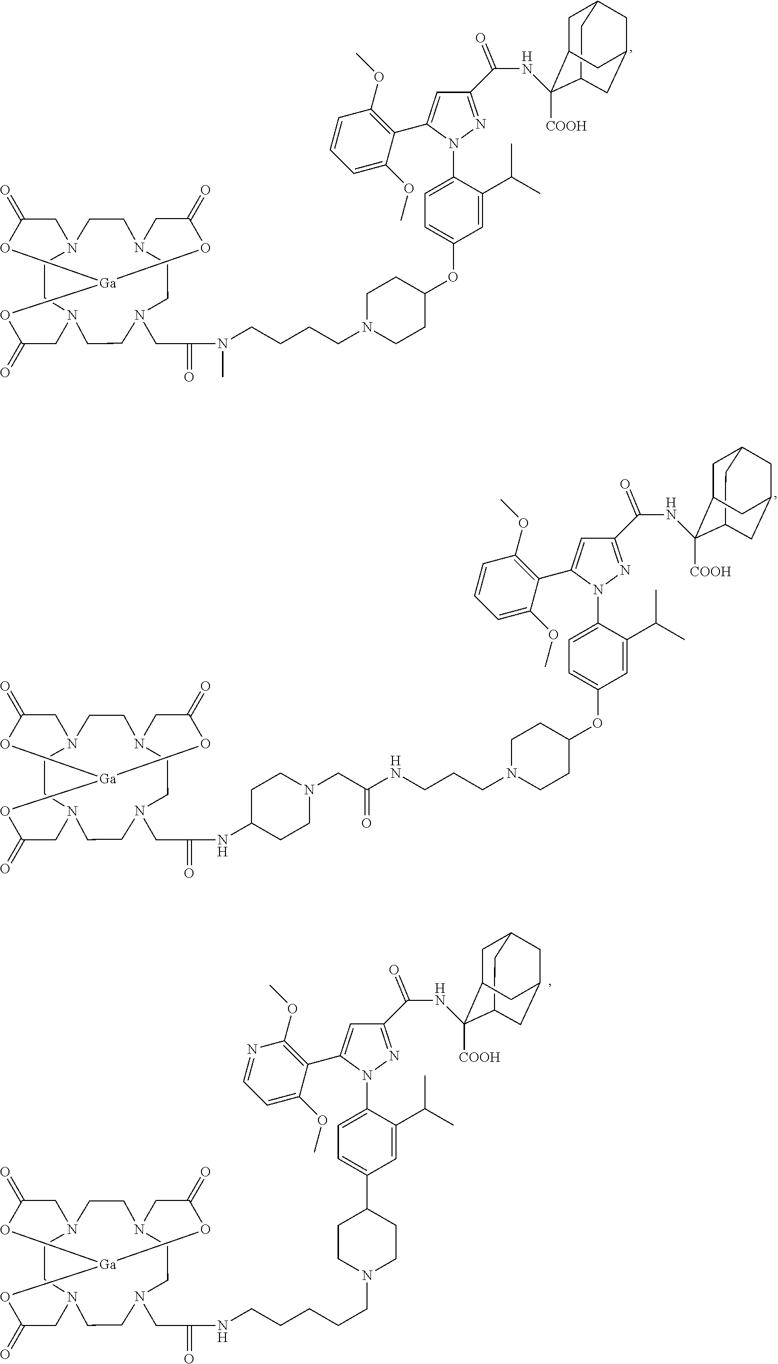

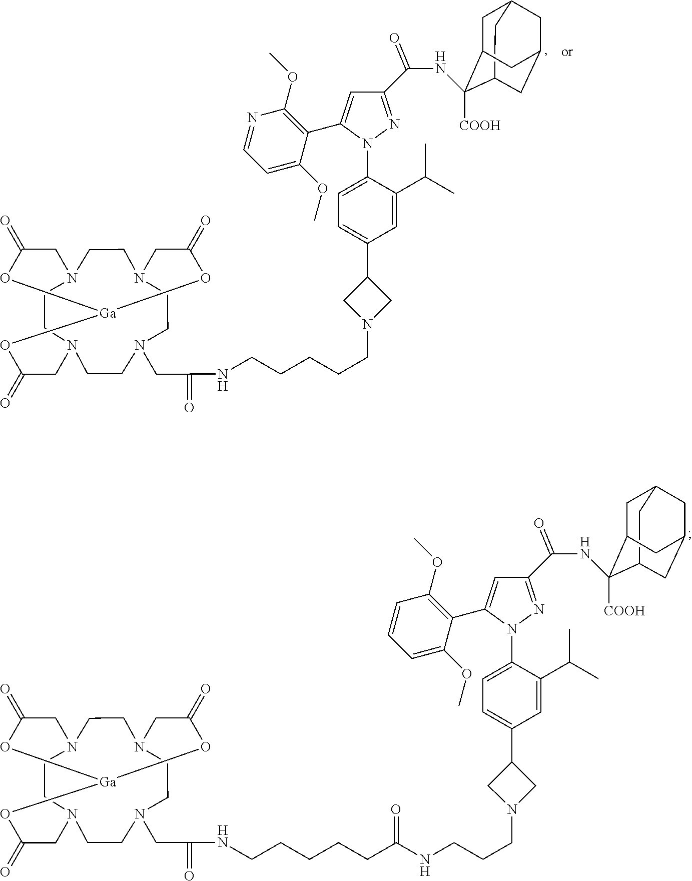

or a pharmaceutically acceptable salt thereof. In some embodiments, the compound of Formula (A) or Formula (I) is:

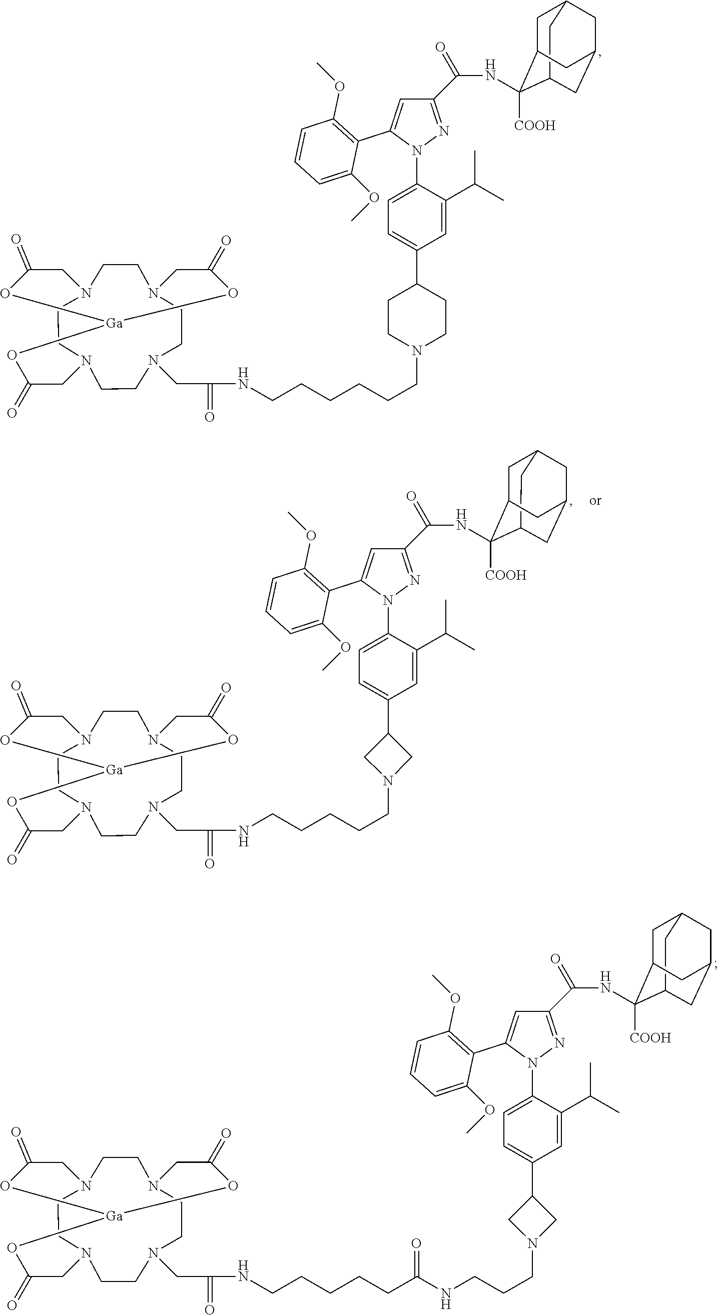

or a pharmaceutically acceptable salt thereof.

In some embodiments, the compound of Formula (A) or Formula (I) is compound 13, a pharmaceutically acceptable salt thereof, or a radionuclide complex thereof; compound 14, a pharmaceutically acceptable salt thereof, or a radionuclide complex thereof; compound 15, a pharmaceutically acceptable salt thereof, or a radionuclide complex thereof; compound 15-Lu or a pharmaceutically acceptable salt thereof; compound 15-Ga or a pharmaceutically acceptable salt thereof compound 16, a pharmaceutically acceptable salt thereof, or a radionuclide complex thereof; compound 17, a pharmaceutically acceptable salt thereof, or a radionuclide complex thereof; compound 18, a pharmaceutically acceptable salt thereof, or a radionuclide complex thereof; compound 19, a pharmaceutically acceptable salt thereof, or a radionuclide complex thereof; compound 20, a pharmaceutically acceptable salt thereof, or a radionuclide complex thereof; compound 21, a pharmaceutically acceptable salt thereof, or a radionuclide complex thereof; compound 22, a pharmaceutically acceptable salt thereof, or a radionuclide complex thereof; compound 23, a pharmaceutically acceptable salt thereof, or a radionuclide complex thereof; compound 24, a pharmaceutically acceptable salt thereof, or a radionuclide complex thereof; compound 24-Lu, or a pharmaceutically acceptable salt thereof; compound 25, a pharmaceutically acceptable salt thereof, or a radionuclide complex thereof; compound 26, a pharmaceutically acceptable salt thereof, or a radionuclide complex thereof; compound 27, a pharmaceutically acceptable salt thereof, or a radionuclide complex thereof; compound 28, a pharmaceutically acceptable salt thereof, or a radionuclide complex thereof; compound 29, a pharmaceutically acceptable salt thereof, or a radionuclide complex thereof; compound 30, a pharmaceutically acceptable salt thereof, or a radionuclide complex thereof; compound 31, a pharmaceutically acceptable salt thereof, or a radionuclide complex thereof; compound 32, a pharmaceutically acceptable salt thereof, or a radionuclide complex thereof; compound 32-Lu, or a pharmaceutically acceptable salt thereof; compound 33, a pharmaceutically acceptable salt thereof, or a radionuclide complex thereof; compound 34, a pharmaceutically acceptable salt thereof, or a radionuclide complex thereof; compound 35, a pharmaceutically acceptable salt thereof, or a radionuclide complex thereof; compound 36, a pharmaceutically acceptable salt thereof, or a radionuclide complex thereof; compound 37, a pharmaceutically acceptable salt thereof, or a radionuclide complex thereof; compound 38, a pharmaceutically acceptable salt thereof, or a radionuclide complex thereof; compound 39, a pharmaceutically acceptable salt thereof, or a radionuclide complex thereof; compound 40, a pharmaceutically acceptable salt thereof, or a radionuclide complex thereof; compound 41, a pharmaceutically acceptable salt thereof, or a radionuclide complex thereof; compound 42, a pharmaceutically acceptable salt thereof, or a radionuclide complex thereof; compound 43, a pharmaceutically acceptable salt thereof, or a radionuclide complex thereof; compound 44, a pharmaceutically acceptable salt thereof, or a radionuclide complex thereof; compound 45, a pharmaceutically acceptable salt thereof, or a radionuclide complex thereof; compound 46, a pharmaceutically acceptable salt thereof, or a radionuclide complex thereof; compound 47, a pharmaceutically acceptable salt thereof, or a radionuclide complex thereof; compound 48, a pharmaceutically acceptable salt thereof, or a radionuclide complex thereof; compound 49, a pharmaceutically acceptable salt thereof, or a radionuclide complex thereof; compound 50, a pharmaceutically acceptable salt thereof, or a radionuclide complex thereof; compound 51, a pharmaceutically acceptable salt thereof, or a radionuclide complex thereof; compound 52, a pharmaceutically acceptable salt thereof, or a radionuclide complex thereof; compound 53, a pharmaceutically acceptable salt thereof, or a radionuclide complex thereof; compound 54, a pharmaceutically acceptable salt thereof, or a radionuclide complex thereof; compound 55, a pharmaceutically acceptable salt thereof, or a radionuclide complex thereof; compound 55-Lu, or a pharmaceutically acceptable salt thereof; compound 56, a pharmaceutically acceptable salt thereof, or a radionuclide complex thereof; compound 57, a pharmaceutically acceptable salt thereof, or a radionuclide complex thereof; compound 57-Lu, or a pharmaceutically acceptable salt thereof; compound 58, a pharmaceutically acceptable salt thereof, or a radionuclide complex thereof; compound 59, a pharmaceutically acceptable salt thereof, or a radionuclide complex thereof; compound 60, a pharmaceutically acceptable salt thereof, or a radionuclide complex thereof; compound 61, a pharmaceutically acceptable salt thereof, or a radionuclide complex thereof; compound 62, a pharmaceutically acceptable salt thereof, or a radionuclide complex thereof; compound 63, a pharmaceutically acceptable salt thereof, or a radionuclide complex thereof; compound 64, a pharmaceutically acceptable salt thereof, or a radionuclide complex thereof; compound 65, a pharmaceutically acceptable salt thereof, or a radionuclide complex thereof; compound 66, a pharmaceutically acceptable salt thereof, or a radionuclide complex thereof; compound 67, a pharmaceutically acceptable salt thereof, or a radionuclide complex thereof; compound 68, a pharmaceutically acceptable salt thereof, or a radionuclide complex thereof; compound 69, a pharmaceutically acceptable salt thereof, or a radionuclide complex thereof; compound 70, a pharmaceutically acceptable salt thereof, or a radionuclide complex thereof; compound 71, a pharmaceutically acceptable salt thereof, or a radionuclide complex thereof; compound 72, a pharmaceutically acceptable salt thereof, or a radionuclide complex thereof; compound 73, a pharmaceutically acceptable salt thereof, or a radionuclide complex thereof; compound 74, a pharmaceutically acceptable salt thereof, or a radionuclide complex thereof; compound 75, a pharmaceutically acceptable salt thereof, or a radionuclide complex thereof; compound 84, a pharmaceutically acceptable salt thereof, or a radionuclide complex thereof; compound 85, a pharmaceutically acceptable salt thereof, or a radionuclide complex thereof; compound 86, a pharmaceutically acceptable salt thereof, or a radionuclide complex thereof; compound 87, a pharmaceutically acceptable salt thereof, or a radionuclide complex thereof; compound 88, a pharmaceutically acceptable salt thereof, or a radionuclide complex thereof; compound 89, a pharmaceutically acceptable salt thereof, or a radionuclide complex thereof; compound 90, a pharmaceutically acceptable salt thereof, or a radionuclide complex thereof; compound 91, a pharmaceutically acceptable salt thereof, or a radionuclide complex thereof; compound 92, a pharmaceutically acceptable salt thereof, or a radionuclide complex thereof; compound 93, a pharmaceutically acceptable salt thereof, or a radionuclide complex thereof; compound 94, a pharmaceutically acceptable salt thereof, or a radionuclide complex thereof; compound 95, a pharmaceutically acceptable salt thereof, or a radionuclide complex thereof; compound 96, a pharmaceutically acceptable salt thereof, or a radionuclide complex thereof; compound 97, a pharmaceutically acceptable salt thereof, or a radionuclide complex thereof; compound 98, a pharmaceutically acceptable salt thereof, or a radionuclide complex thereof; compound 99, a pharmaceutically acceptable salt thereof, or a radionuclide complex thereof; compound 99-Lu, or a pharmaceutically acceptable salt thereof; compound 100, a pharmaceutically acceptable salt thereof, or a radionuclide complex thereof; compound 101, a pharmaceutically acceptable salt thereof, or a radionuclide complex thereof; compound 102, a pharmaceutically acceptable salt thereof, or a radionuclide complex thereof; compound 103, a pharmaceutically acceptable salt thereof, or a radionuclide complex thereof; compound 104, a pharmaceutically acceptable salt thereof, or a radionuclide complex thereof; compound 105, a pharmaceutically acceptable salt thereof, or a radionuclide complex thereof; compound 106, a pharmaceutically acceptable salt thereof, or a radionuclide complex thereof; compound 107, a pharmaceutically acceptable salt thereof, or a radionuclide complex thereof; compound 108, a pharmaceutically acceptable salt thereof, or a radionuclide complex thereof; compound 109, a pharmaceutically acceptable salt thereof, or a radionuclide complex thereof; compound 110, a pharmaceutically acceptable salt thereof, or a radionuclide complex thereof; compound 111, a pharmaceutically acceptable salt thereof, or a radionuclide complex thereof; compound 112, a pharmaceutically acceptable salt thereof, or a radionuclide complex thereof; compound 113, a pharmaceutically acceptable salt thereof, or a radionuclide complex thereof; compound 114, a pharmaceutically acceptable salt thereof, or a radionuclide complex thereof; compound 115, a pharmaceutically acceptable salt thereof, or a radionuclide complex thereof; compound 116, a pharmaceutically acceptable salt thereof, or a radionuclide complex thereof; compound 116-Lu, or a pharmaceutically acceptable salt thereof; compound 116-Ga, or a pharmaceutically acceptable salt thereof; compound 117, a pharmaceutically acceptable salt thereof, or a radionuclide complex thereof; compound 118, a pharmaceutically acceptable salt thereof, or a radionuclide complex thereof; compound 119, a pharmaceutically acceptable salt thereof, or a radionuclide complex thereof; compound 120, a pharmaceutically acceptable salt thereof, or a radionuclide complex thereof; compound 121, a pharmaceutically acceptable salt thereof, or a radionuclide complex thereof; compound 122, a pharmaceutically acceptable salt thereof, or a radionuclide complex thereof; compound 123, a pharmaceutically acceptable salt thereof, or a radionuclide complex thereof; compound 124, a pharmaceutically acceptable salt thereof, or a radionuclide complex thereof; compound 137, a pharmaceutically acceptable salt thereof, or a radionuclide complex thereof; compound 138, a pharmaceutically acceptable salt thereof, or a radionuclide complex thereof; compound 139, a pharmaceutically acceptable salt thereof, or a radionuclide complex thereof; compound 140, a pharmaceutically acceptable salt thereof, or a radionuclide complex thereof; compound 141, a pharmaceutically acceptable salt thereof, or a radionuclide complex thereof; compound 141-Lu or a pharmaceutically acceptable salt thereof; compound 141-Ga or a pharmaceutically acceptable salt thereof; compound 142, a pharmaceutically acceptable salt thereof, or a radionuclide complex thereof; compound 143, a pharmaceutically acceptable salt thereof, or a radionuclide complex thereof; compound 144, a pharmaceutically acceptable salt thereof, or a radionuclide complex thereof; compound 145, a pharmaceutically acceptable salt thereof, or a radionuclide complex thereof; compound 146, a pharmaceutically acceptable salt thereof, or a radionuclide complex thereof; compound 147, a pharmaceutically acceptable salt thereof, or a radionuclide complex thereof; compound 148, a pharmaceutically acceptable salt thereof, or a radionuclide complex thereof; compound 149, a pharmaceutically acceptable salt thereof, or a radionuclide complex thereof; compound 150, a pharmaceutically acceptable salt thereof, or a radionuclide complex thereof; compound 151, a pharmaceutically acceptable salt thereof, or a radionuclide complex thereof; compound 152, a pharmaceutically acceptable salt thereof, or a radionuclide complex thereof; compound 153, a pharmaceutically acceptable salt thereof, or a radionuclide complex thereof; compound 154, a pharmaceutically acceptable salt thereof, or a radionuclide complex thereof; compound 155, a pharmaceutically acceptable salt thereof, or a radionuclide complex thereof; compound 156, a pharmaceutically acceptable salt thereof, or a radionuclide complex thereof; compound 157, a pharmaceutically acceptable salt thereof, or a radionuclide complex thereof; compound 158, a pharmaceutically acceptable salt thereof, or a radionuclide complex thereof; compound 159, a pharmaceutically acceptable salt thereof, or a radionuclide complex thereof; or compound 160, a pharmaceutically acceptable salt thereof, or a radionuclide complex thereof.

Any combination of the groups described above for the various variables is contemplated herein. Throughout the specification, groups and substituents thereof are chosen by one skilled in the field to provide stable moieties and compounds.

### Representative Ligand Compounds

In one aspect, described herein is a compound of Formula (D), or a pharmaceutically acceptable salt thereof:

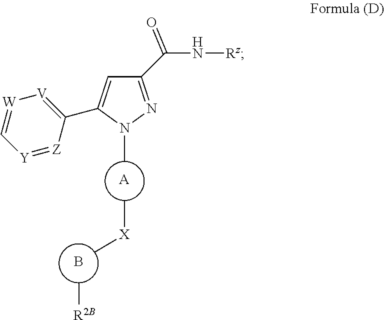

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
   each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
   R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)—, ═S(═NH)(═NH), —CH═CH—C(═O)—, or —CH₂CH₂—C(═O)—;
   each R^{2A} is independently selected from the group consisting of hydrogen, —F, —Cl, —Br, —I, —OH, —O—C₁-C₆ alkyl, substituted or unsubstituted C₁-C₆ alkyl, C₁-C₆ fluoroalkyl, substituted or unsubstituted heteroalkyl, —CN, —CO₂H, —C(═O)NR^{3A}R^{4A}, —CH(═N)OH, —NR^{5A}R^{6A}, —NO₂, —NR^{7A}(═O)R^{8A}, —NR^{9A}C(═)OR^{10A}, and —OC(═O)NR^{11A}R^{12A}, or two R^{2A} variables on the same or adjacent carbon atoms come together to form an unsubstituted C₃-C₆ cycloalkyl ring;
   R^{3A}, R^{4A}, R^{5A}, R^{6A}, R^{7A}, R^{8A}, R^{9A}, R^{10A}, R^{11A} and R^{12A} are each independently —H or —CH₃;
R^{2B} is hydrogen, —R^{b}, —U—R^{c}, or —NH—R^{d},
   R^{b} is hydrogen, C₁-C₆ alkyl or 6-membered heteroalkyl;
   U is —O— or —NH—;
   R^{c} is hydrogen or C₁-C₆ alkyl;
   R^{d} is C₁-C₆ alkyl;
W is —C(R^{r})— or —N— and Y is —C(R^{t})— or —N—; wherein R^{r} and R^{t} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—; wherein R^{x} and R^{y} are each independently selected from the group consisting of: —F, —Cl, —Br, —CF₃, —C₁-C₆ alkyl, —O—C₁-C₆ alkyl, —C₃-C₆ cycloalkyl, aryl and heteroaryl.
R^{z} is

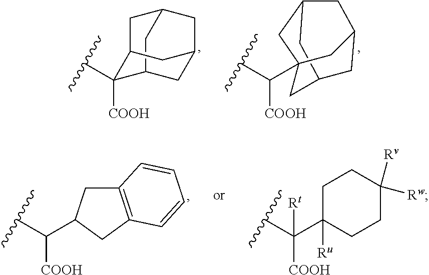

R^{v} and R^{w} are each independently —F, —Cl, or —CH₃ and R^{t} and R^{u} are each independently —C₁-C₄ alkyl; or
R^{v} is —F, —Cl, or —CH₃; R^{t} is —C₁-C₄ alkyl; and R^{t} and R^{u} come together with the carbon atoms to which they are attached to form a cyclopentyl or cyclohexyl ring.

In some embodiments, W is —N— and Y is —CH—. In some embodiments, W and Y are —CH—.

In some embodiments, V is —C(R^{x})— and Z is —C(R^{y})—.

In some embodiments, R^{x} is —F, —Cl, —CF₃, —CH₃, —CH₂CH₃, —OCH₃, cyclopropyl, or 5 to 6-membered heteroaryl. In some embodiments, R^{x} is pyrimidinyl.

In some embodiments, R^{y} is —OCH³.

In some embodiments, R^{x} and R^{y} are each independently selected from the group consisting of: —F, —Cl, —Br, —CF₃, —C₁-C₆ alkyl, and —O—C₁-C₆ alkyl. In some embodiments, R^{x} is —F, —Cl, —CF₃, —CH₃, —CH₂CH₃, —OCH₃, cyclopropyl, or pyrimidinyl and R^{y} is —OCH₃. In some embodiments, R^{x} is —F, —Cl, or —OCH₃, and R^{y} is —OCH₃. In some embodiments, R^{x} and R^{y} are —OCH₃.

In some embodiments, W is —CH— or —N—; Y is —CH—; R^{x} is —OCH₃, and R^{y} is —OCH₃.

In some embodiments, V is —C(R^{x})—; Z is —C(R^{y})—; R^{x} is selected from the group consisting of: —F, —Cl, —CF₃, —C₁-C₆ alkyl, —O—C₁-C₆ alkyl, —C₃-C₆ cycloalkyl, and heteroaryl; and R^{y} is —O—C₁-C₆ alkyl.

In some embodiments, V is —C(R^{x})—; Z is —C(R^{y})—; and R^{x} and R^{y} are each independently —O—C₁-C₆ alkyl.

In some embodiments, R^{z} is

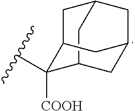 [SMILES: *C1(C(=O)O)C2CC3CC(C2)CC1C3]

In some embodiments, R^{z} is

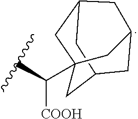 [SMILES: *[C@H](C(=O)O)C12CC3CC(CC(C3)C1)C2]

In some embodiments, R^{z} is

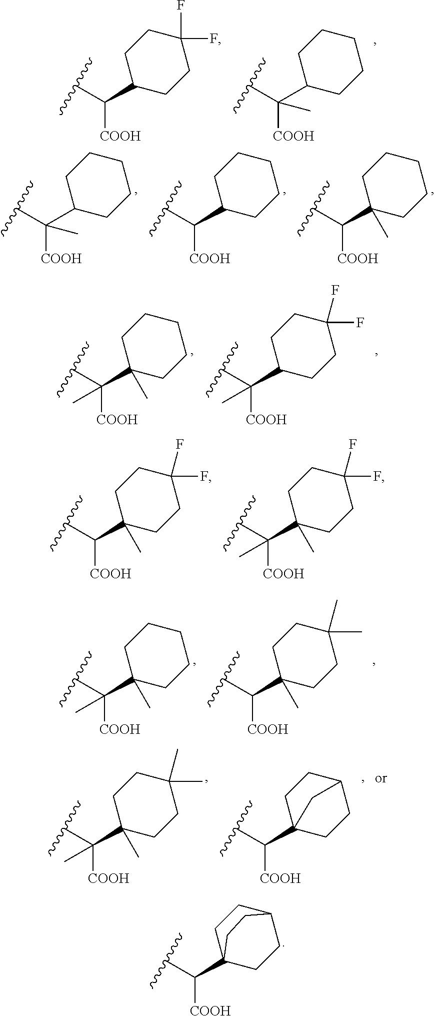

In some embodiments, R^{z} is

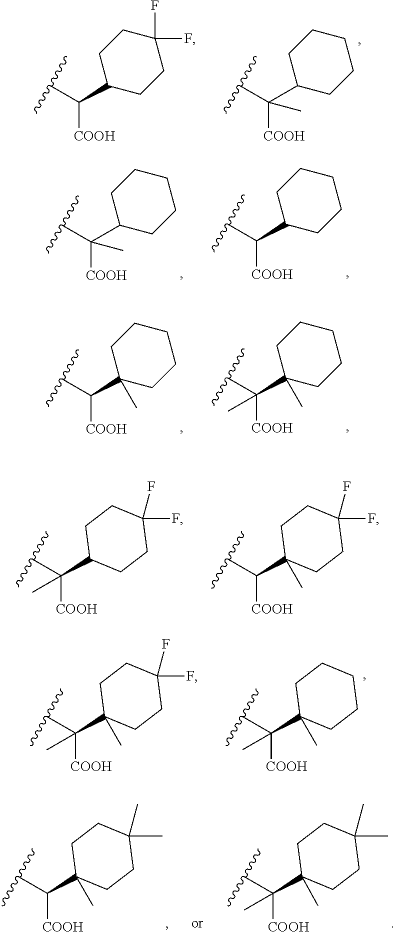

In some embodiments, R^{z} is

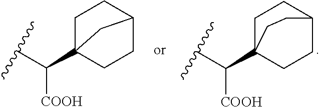 [SMILES: *[C@H](C(=O)O)C12CCC(CC1)C2 ; *[C@H](C(=O)O)C12CCC(CC1)CC2]

In some embodiments, R^{z} is

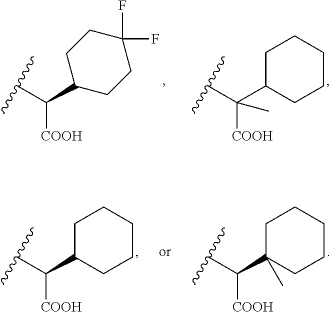 [SMILES: *[C@H](C(=O)O)C1CCC(F)(F)CC1 ; *C(C)(C(=O)O)C1CCCCC1 ; *[C@H](C(=O)O)C1CCCCC1 ; *[C@H](C(=O)O)C1(C)CCCCC1]

In some embodiments, R^{z} is

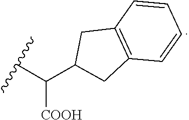 [SMILES: *C(C(=O)O)C1Cc2ccccc2C1]

In some embodiments, the compound of Formula (D) is a compound of Formula (E), or a pharmaceutically acceptable salt thereof:

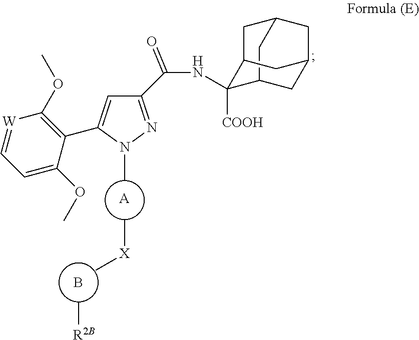

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O).
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O). wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
   R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)═S(═NH)(═NH), —CH═CH—C(═O)—, or —CH₂CH₂—C(═O)—;
each R^{2A} is independently selected from the group consisting of hydrogen, —F, —Cl, —Br, —I, —OH, —O—C₁-C₆ alkyl, substituted or unsubstituted C₁-C₆ alkyl, C₁-C₆ fluoroalkyl, substituted or unsubstituted heteroalkyl, —CN, —CO₂H, —C(═O)NR^{3A}R^{4A}, —CH(═N)OH, —NR^{5A}R^{6A}, —NO₂, —NR^{7A}C(═O)R^{8A}, —NR^{9A}C(═O)OR^{10A}, and —OC(═O)NR^{11A}R^{12A};
   R^{3A}, R^{4A}, R^{5A}, R^{6A}, R^{7A}, R^{8A}, R^{9A}, R^{10A}, R^{11A} and R^{12A} are each independently —H or —CH₃;
R^{2B} is hydrogen, —R^{b}, —U—R^{c}, or —NH—R^{d},
   R^{b} is hydrogen, C₁-C₆ alkyl or 6-membered heteroalkyl;
   U is —O— or —NH—;
   R^{c} is hydrogen or C₁-C₆ alkyl;
   R^{d} is C₁-C₆ alkyl; and
W is —C(H)—, —C(F)—, —C(OMe)-, —C(OEt)-, or —N—.

In some embodiments, Ring A is phenyl, naphthyl, or a bicyclic heteroaryl ring; wherein each Ring A is optionally substituted with 1-6 R¹ substituents. In some embodiments, Ring A is phenyl, naphthyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, or 1,5-naphthyridinyl; wherein each Ring A is optionally substituted with 1-6 R¹ substituents. In some embodiments, Ring A is a partially unsaturated bicyclic heterocyclic ring; wherein any saturated carbon of Ring A is optionally substituted with oxo (═O) and wherein each Ring A is optionally substituted with 1-6 R¹ substituents. In some embodiments, Ring A is phenyl, wherein Ring A is optionally substituted with 1-6 R¹ substituents. In some embodiments, Ring A is naphthyl, wherein Ring A is optionally substituted with 1-6 R¹ substituents.

In some embodiments, Ring B is absent and Ring A is a partially unsaturated bicyclic heterocyclic ring selected from indolinyl, isoindolinyl, 1,2-dihydroisoquinolinyl, 1,2,3,4-tetrahydroquinolinyl, 1,2,3,4-tetrahydroisoquinolinyl, 1,2,3,4-tetrahydroquinoxalinyl, 1,2,3,4-tetrahydro-1,8-naphthyridine, 5,6,7,8-tetrahydro-1,7-naphthyridinyl, 5,6,7,8-tetrahydro-1,6-naphthyridinyl, 5,6,7,8-tetrahydro-1,5-naphthyridinyl, 1,2,3,4-tetrahydro-1,6-naphthyridinyl, 1,2,3,4-tetrahydro-2,6-naphthyridinyl, 1,2,3,4-tetrahydro-3,6-naphthyridinyl, or 1,2,3,4-tetrahydro-4,6-naphthyridinyl, or 2,3-dihydrobenzo[d]oxazolyl; wherein any saturated carbon of Ring A is optionally substituted with oxo (═O) and wherein each Ring A is optionally substituted with 1-6 R¹ substituents. In some embodiments, Ring B is absent and Ring A is 1,2,3,4-tetrahydroisoquinolinyl, wherein Ring A is optionally substituted with 1-6 R¹ substituents.

In some embodiments, Ring A is a partially unsaturated bicyclic heterocyclic ring selected from indolinyl, isoindolinyl, 1,2-dihydroisoquinolinyl, 1,2,3,4-tetrahydroquinolinyl, 1,2,3,4-tetrahydroisoquinolinyl, 1,2,3,4-tetrahydroquinoxalinyl, 1,2,3,4-tetrahydro-1,8-naphthyridine, 5,6,7,8-tetrahydro-1,7-naphthyridinyl, 5,6,7,8-tetrahydro-1,6-naphthyridinyl, 5,6,7,8-tetrahydro-1,5-naphthyridinyl, 1,2,3,4-tetrahydro-1,6-naphthyridinyl, 1,2,3,4-tetrahydro-2,6-naphthyridinyl, 1,2,3,4-tetrahydro-3,6-naphthyridinyl, or 1,2,3,4-tetrahydro-4,6-naphthyridinyl, or 2,3-dihydrobenzo[d]oxazolyl; wherein any saturated carbon of Ring A is optionally substituted with oxo (═O) and wherein each Ring A is optionally substituted with 1-6 R¹ substituents. In some embodiments, Ring A is 1,2,3,4-tetrahydroisoquinolinyl, wherein Ring A is optionally substituted with 1-6 R¹ substituents.

In some embodiments, each R¹ is independently selected from the group consisting of —F, —C₁, —Br, —I, —OH, —CF₃, —CN, —CO₂H, —C(═O)OCH₃, —C(═O)NH₂, —CH(N)O, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, each R¹ is independently selected from the group consisting of —F, —C₁, —Br, —I, —OH and —C₁-C₆ alkyl. In some embodiments, R¹ is —CH(CH₃)₂.

In some embodiments, X is a bond. In some embodiments, X is —O— or —NH—. In some embodiments, X is —S(═O)(═NH)— or ═S(═NH)(═NH)—.

In some embodiments, Ring B is piperidinyl, pyrrolidinyl, or azetidinyl, and R^{2B} is hydrogen, —R^{b}, —U—R^{c}, or —NH—R^{d}; wherein Ring B is optionally substituted with one or more R^{2A} substituents; and wherein any saturated carbon of Ring B is optionally substituted with oxo (═O). In some embodiments, Ring B is substituted or unsubstituted piperazinyl and R^{2B} is hydrogen, —R^{b}, —U—R^{c}, or —NH—R^{d}; wherein Ring B is optionally substituted with one or more R^{2A}substituents. In some embodiments, Ring B is piperidinyl and R^{2B} is hydrogen, —R^{b}, —U—R^{c}, or —NH—R^{d}.

In some embodiments, each R^{2A} is independently selected from the group consisting of —F, substituted or unsubstituted C₁-C₆ alkyl, —CF₃, or —CN. In some embodiments, each R^{2A} is independently —F or substituted or unsubstituted —C₁-C₆ alkyl. In some embodiments, R^{2A} is —CH₂NH₂.

In some embodiments, —X-Ring B— is absent,

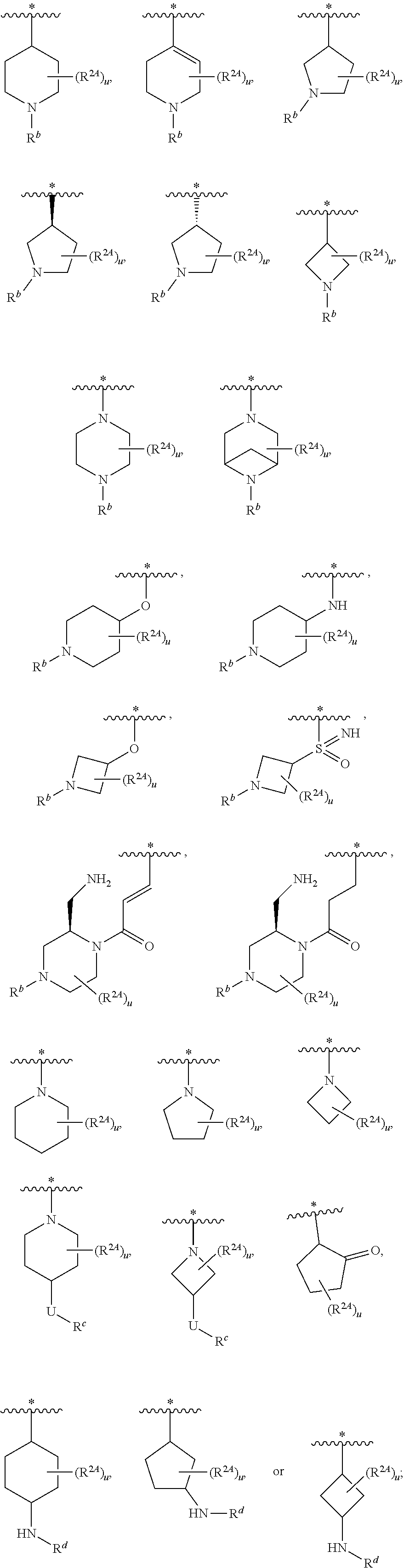

U is —O— or —NH—; R^{b} is hydrogen, C₁-C₆ alkyl or 6-membered heteroalkyl; R^{c} is hydrogen or C₁-C₆ alkyl; and R^{d} is C₁-C₆ alkyl; R^{2A} is hydrogen, —F, —Cl, —Br, —I, —OH, —O—C₁-C₆ alkyl, unsubstituted C₁-C₆ alkyl, or C₁-C₆ fluoroalkyl; and u is 0, 1, 2, 3, or 4; wherein * denotes the attachment point of —X-Ring B— to Ring A.

In some embodiments, —X-Ring B— is absent,

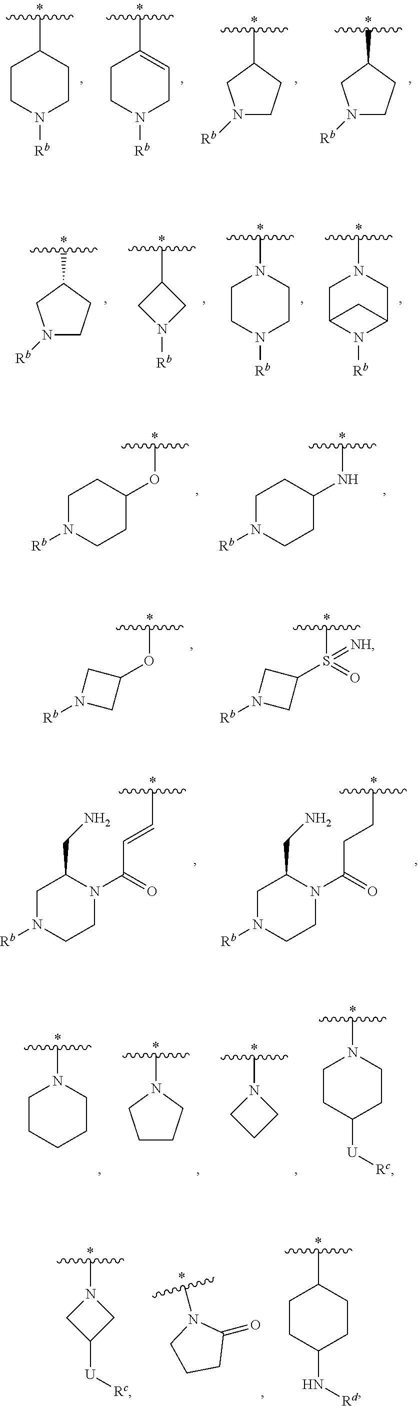

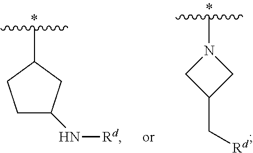

U is —O— or —NH—; R^{b} is hydrogen, C₁-C₆ alkyl or 6-membered heteroalkyl; R^{c} is hydrogen or C₁-C₆ alkyl; and R^{d} is C₁-C₆ alkyl; wherein * denotes the attachment point of —X-Ring B— to Ring A.

In some embodiments, —X-Ring B— is absent. In some embodiments, —X-Ring B— is

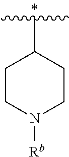

and R^{b} is hydrogen, C₁-C₆ alkyl or 6-membered heteroalkyl; wherein * denotes the attachment point of —X-Ring B— to Ring A. In some embodiments, —X-Ring B— is

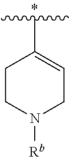

and R^{b} is hydrogen, C₁-C₆ alkyl or 6-membered heteroalkyl; wherein * denotes the attachment point of —X-Ring B— to Ring A. In some embodiments, —X-Ring B— is

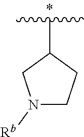

and R^{b} is hydrogen, C₁-C₆ alkyl or 6-membered heteroalkyl; wherein * denotes the attachment point of —X-Ring B— to Ring A. In some embodiments, —X-Ring B— is

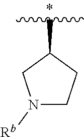

and R^{b} is hydrogen, C₁-C₆ alkyl or 6-membered heteroalkyl; wherein * denotes the attachment point of —X-Ring B— to Ring A. In some embodiments, —X-Ring B— is

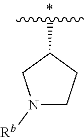

and R^{b} is hydrogen, C₁-C₆ alkyl or 6-membered heteroalkyl; wherein * denotes the attachment point of —X-Ring B— to Ring A. In some embodiments, —X-Ring B— is

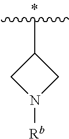

and R^{b} is hydrogen, C₁-C₆ alkyl or 6-membered heteroalkyl; wherein * denotes the attachment point of —X-Ring B— to Ring A. In some embodiments, —X-Ring B— is

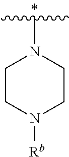

and R^{b} is hydrogen, C₁-C₆ alkyl or 6-membered heteroalkyl; wherein * denotes the attachment point of —X-Ring B— to Ring A. In some embodiments, —X-Ring B— is

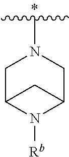

and R^{b} is hydrogen, C₁-C₆ alkyl or 6-membered heteroalkyl; wherein * denotes the attachment point of —X-Ring B— to Ring A. In some embodiments, —X-Ring B— is

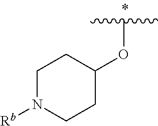

and Rbis hydrogen, C₁-C₆ alkyl or 6-membered heteroalkyl; wherein * denotes the attachment point of —X-Ring B— to Ring A. In some embodiments, —X-Ring B— is

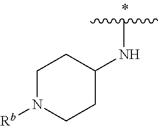

and R^{b} is hydrogen, C₁-C₆ alkyl or 6-membered heteroalkyl; wherein * denotes the attachment point of —X-Ring B— to Ring A. In some embodiments, —X-Ring B— is

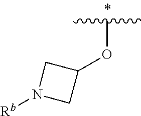

and R^{b} is hydrogen, C₁-C₆ alkyl or 6-membered heteroalkyl; wherein * denotes the attachment point of —X-Ring B— to Ring A. In some embodiments, —X-Ring B— is

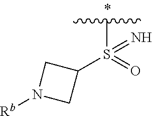

and R^{b} is hydrogen, C₁-C₆ alkyl or 6-membered heteroalkyl; wherein * denotes the attachment point of —X-Ring B— to Ring A. In some embodiments, —X-Ring B— is

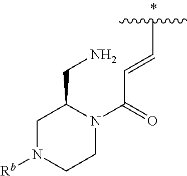

and R^{b} is hydrogen, C₁-C₆ alkyl or 6-membered heteroalkyl; wherein * denotes the attachment point of —X-Ring B— to Ring A. In some embodiments, —X-Ring B— is

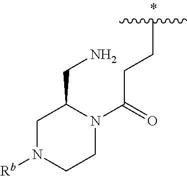

and R^{b} is hydrogen, C₁-C₆ alkyl or 6-membered heteroalkyl; wherein * denotes the attachment point of —X-Ring B— to Ring A. In some embodiments, —X-Ring B— is

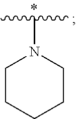 [SMILES: *N1CCCCC1]

wherein * denotes the attachment point of —X-Ring B— to Ring A. In some embodiments, —X-Ring B— is

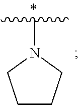 [SMILES: *N1CCCC1]

wherein * denotes the attachment point of —X-Ring B— to Ring A. In some embodiments, —X-Ring B— is

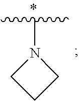 [SMILES: *N1CCC1]

wherein * denotes the attachment point of —X-Ring B— to Ring A. In some embodiments, —X-Ring B— is

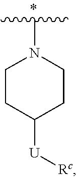

U is —O— or —NH—, and R^{c} is hydrogen or C₁-C₆ alkyl; wherein * denotes the attachment point of —X-Ring B— to Ring A. In some embodiments, —X-Ring B— is

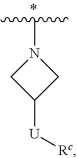

U is —O— or —NH—, and R^{c} is hydrogen or C₁-C₆ alkyl; wherein * denotes the attachment point of —X-Ring B— to Ring A. In some embodiments, —X-Ring B— is

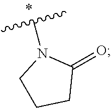 [SMILES: *N1CCCC1=O]

wherein * denotes the attachment point of —X-Ring B— to Ring A. In some embodiments, —X-Ring B— is

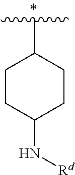

and R^{d} is C₁-C₆ alkyl; wherein * denotes the attachment point of —X-Ring B— to Ring A. In some embodiments, —X-Ring B— is

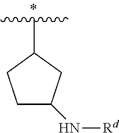

and R^{d} is C₁-C₆ alkyl; wherein * denotes the attachment point of —X-Ring B— to Ring A. In some embodiments, —X -Ring B— is

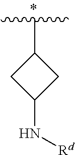

and R^{d} is C₁-C₆ alkyl; wherein * denotes the attachment point of —X-Ring B— to Ring A.

In some embodiments, Ring A-X-Ring B is:

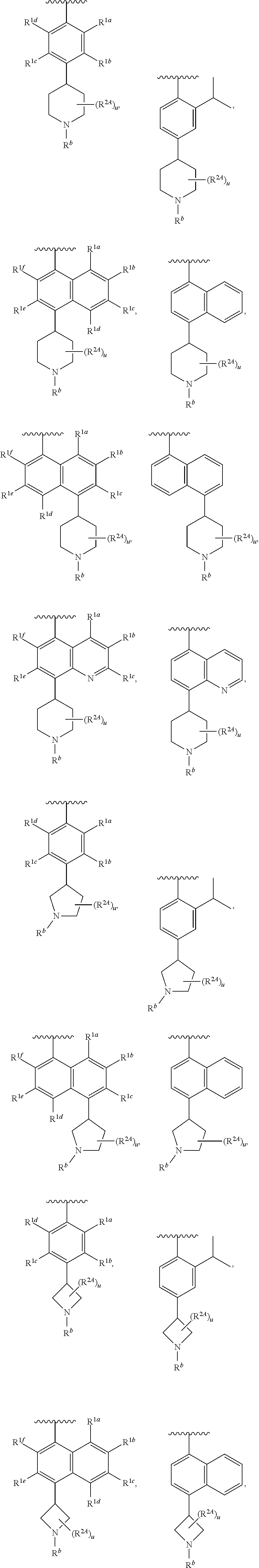

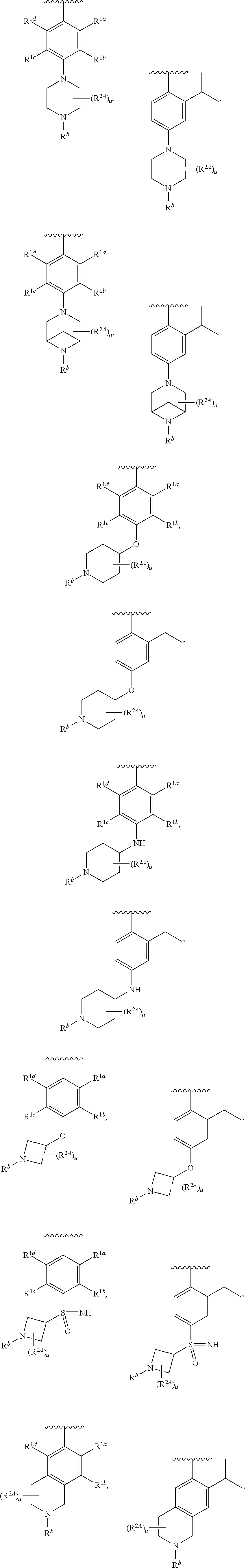

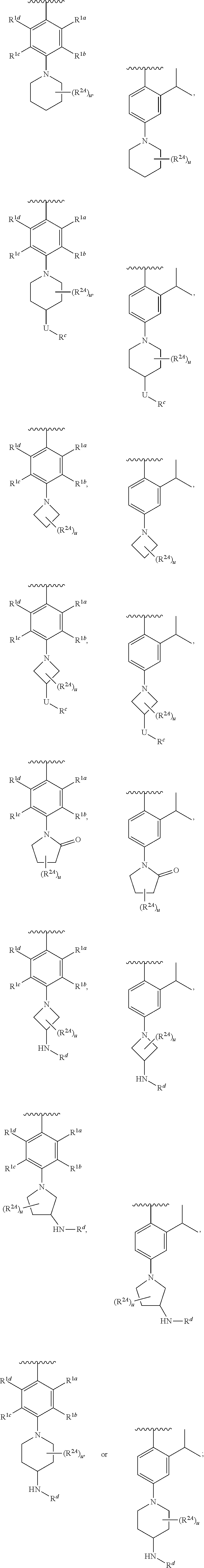

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; U is —O— or —NH—; R^{b} is hydrogen, C₁-C₆ alkyl or 6-membered heteroalkyl; R^{c} is hydrogen or C₁-C₆ alkyl; R^{2A} is hydrogen, —F, —Cl, —Br, —I, —OH, —O—C₁-C₆ alkyl, C₁-C₆ alkyl, or C₁-C₆ fluoroalkyl; and u is 0, 1, 2, 3, or 4.

In some embodiments, Ring A-X-Ring B is:

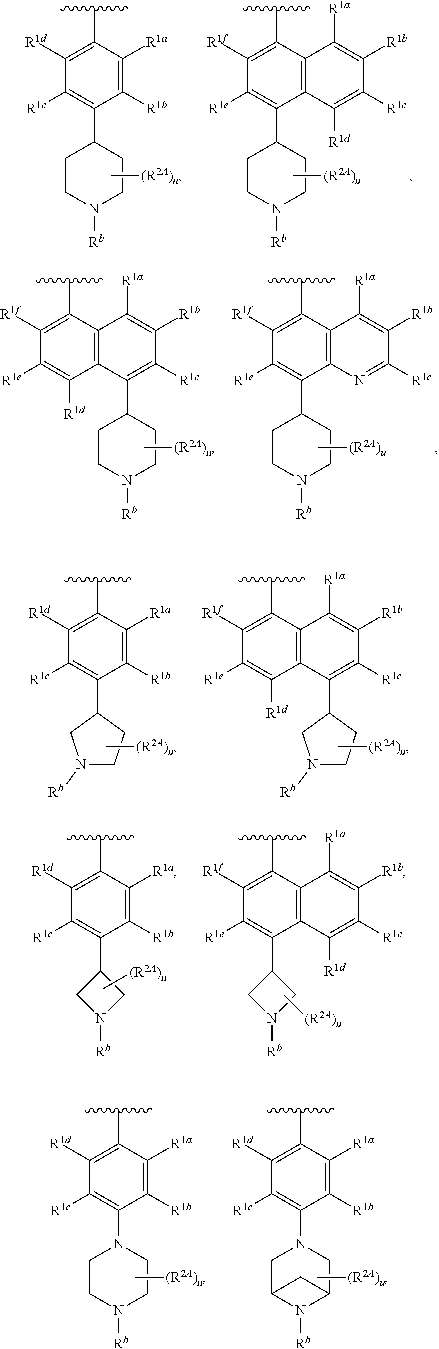

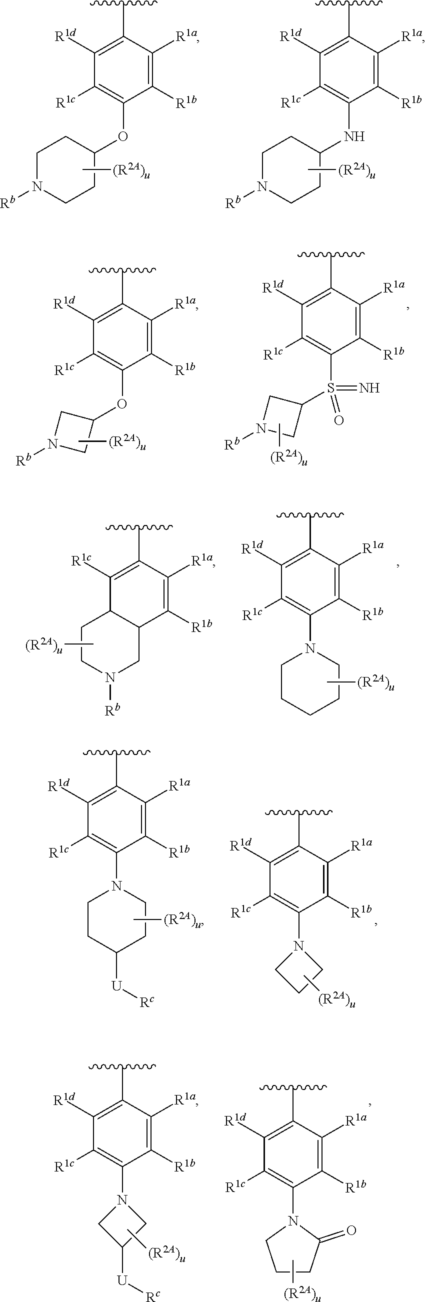

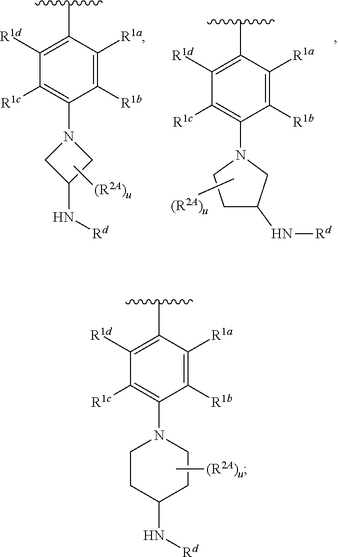

wherein R^{1a}, R^{1b}, R^{1c}, R^{1d}, R^{1e}, and R^{1f} are each independently selected from the group consisting of hydrogen, —F, —Cl, —Br, -L, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; U is —O— or —NH—; Rbis hydrogen, C₁-C₆ alkyl or 6-membered heteroalkyl; R^{c} is hydrogen or C₁—C alkyl; R² is hydrogen, —F, —Cl, —Br, —I, —OH, —O—C₁-C₆ alkyl, C₁-C₆ alkyl, or C₁-C₆ fluoroalkyl; and u is 0, 1, 2, 3, or 4.

In some embodiments, Ring A-X-Ring B is:

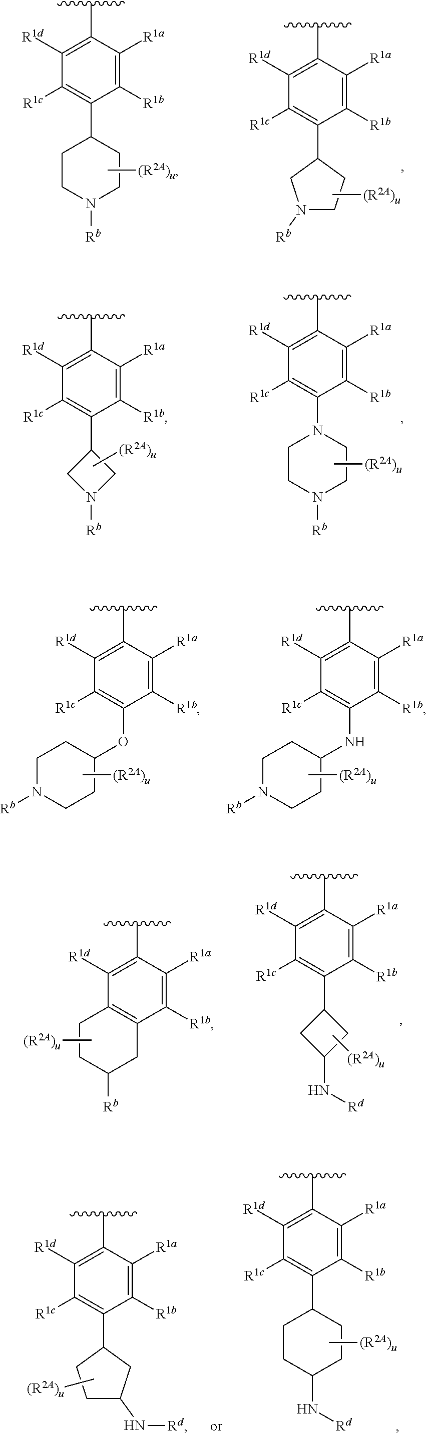

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; U is —O— or —NH—; R^{b} is hydrogen, substituted or unsubstituted C₁-C₆ alkyl or substituted or unsubstituted 6-membered heteroalkyl; R^{c} is hydrogen or C₁C alkyl; R^{d} is C₁-C₆ alkyl; R² is hydrogen, —F, —Cl, —Br, -, —OH, —O—C₁-C₆ alkyl, unsubstituted C₁-C₆ alkyl, or C₁-C₆ fluoroalkyl; and u is 0, 1, 2, 3, or 4. In some embodiments, u is 0.

In some embodiments, Ring A-X-Ring B is:

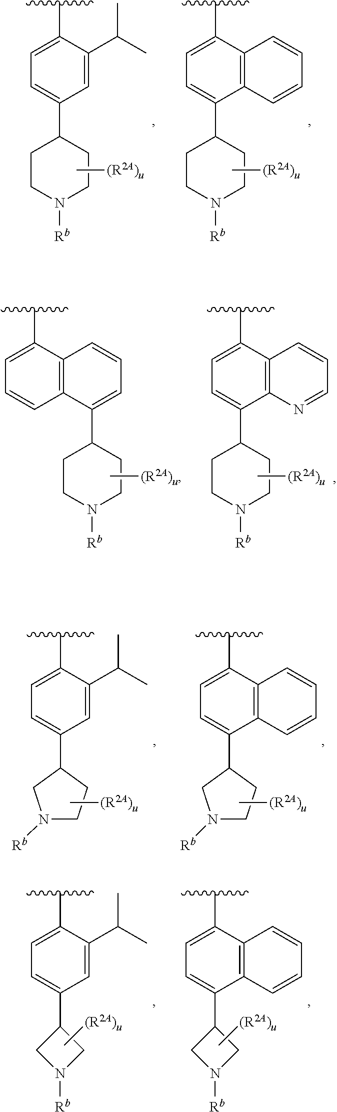

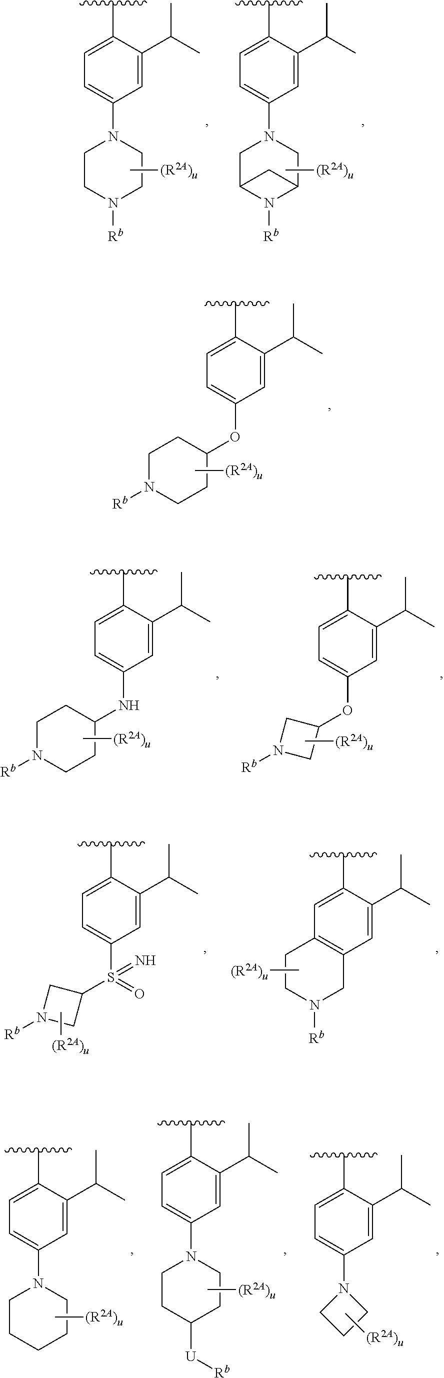

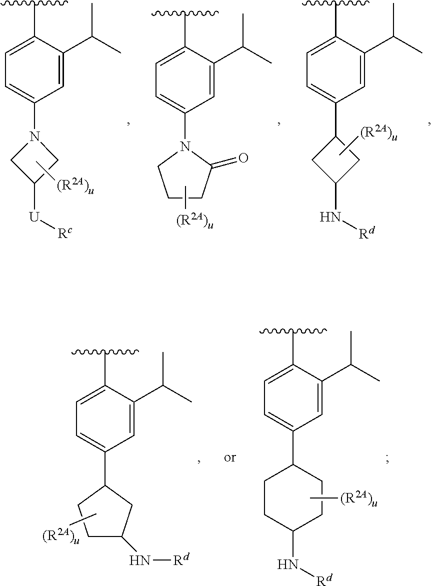

wherein U is —O— or —NH—; R^{b} is hydrogen, C₁-C₆ alkyl or 6-membered heteroalkyl; R^{c} is hydrogen or C₁-C₆ alkyl; R² is hydrogen, —F, —Cl, —Br, —I, —OH, —O—C₁-C₆ alkyl, C₁-C₆ alkyl, or C₁-C₆ fluoroalkyl; and u is 0, 1, 2, 3, or 4.

In some embodiments, Ring A-X-Ring B is:

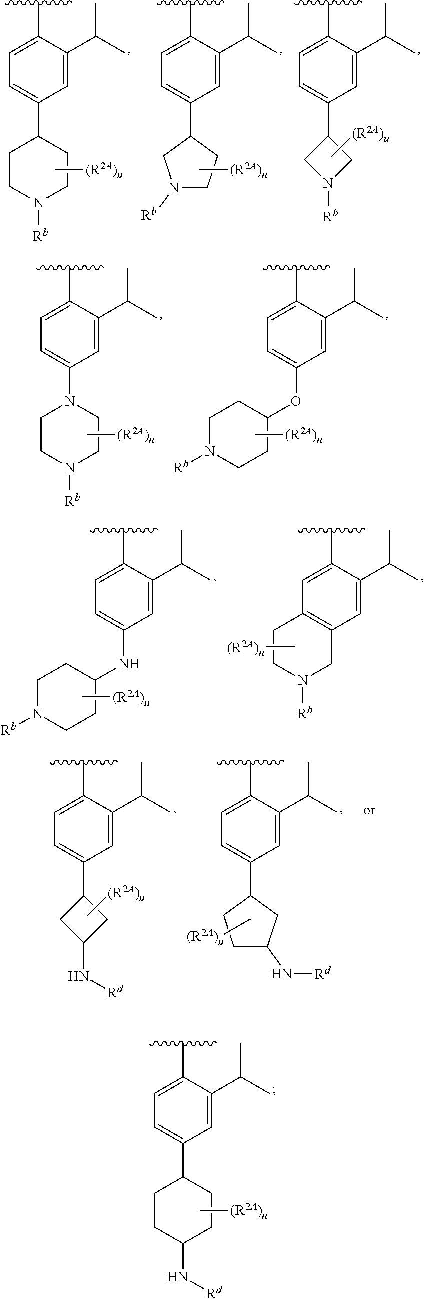

wherein U is —O— or —NH—; R^{b} is hydrogen, substituted or unsubstituted C₁-C₆ alkyl or substituted or unsubstituted 6-membered heteroalkyl; R^{c} is hydrogen or C₁-C₆ alkyl; R⁴ is C₁-C₆ alkyl; R^{2A} is hydrogen, —F, —Cl, —Br, —I, —OH, —O—C₁-C₆ alkyl, unsubstituted C₁-C₆ alkyl, or C₁-C₆ fluoroalkyl; and u is 0, 1, 2, 3, or 4. In some embodiments, u is 0.

In some embodiments, R^{2A} is hydrogen, —F, —Cl, —Br, —I, —OH, —O—C₁-C₆ alkyl, unsubstituted C₁-C₆ alkyl, or C₁-C₆ fluoroalkyl. In some embodiments, R^{2A} is hydrogen. In some embodiments, R^{2A} is —F. In some embodiments, R^{2A} is —C₁. In some embodiments, R⁹ is —Br. In some embodiments, R^{2A} is —I. In some embodiments, R^{2A} is —OH. In some embodiments, R^{2A} is —O—C₁-C₆ alkyl. In some embodiments, R^{2A} is unsubstituted C₁-C₆ alkyl. In some embodiments, R^{2A} is C₁-C₆ fluoroalkyl.

In some embodiments, R^{3A} is H. In some embodiments, R^{3A} is —CH₃. In some embodiments, R^{4A} is H. In some embodiments, R^{4A} is —CH₃. In some embodiments, R^{A} is H. In some embodiments, R^{5A} is —CH₃. In some embodiments, R^{6A} is H. In some embodiments, R^{6A} is —CH₃. In some embodiments, R^{7A} is H. In some embodiments, R^{7A} is —CH₃. In some embodiments, R^{8A} is H. In some embodiments, R^{8A} is —CH₃. In some embodiments, R^{9A} is H. In some embodiments, R^{9A} is —CH₃. In some embodiments, R^{10A} is H. In some embodiments, R^{10A} is —CH₃. In some embodiments, R^{11A} is H. In some embodiments, R^{11A} is —CH₃. In some embodiments, R^{12A} is H. In some embodiments, R^{12A} is —CH₃.

In some embodiments, Ring A-X-Ring B is:

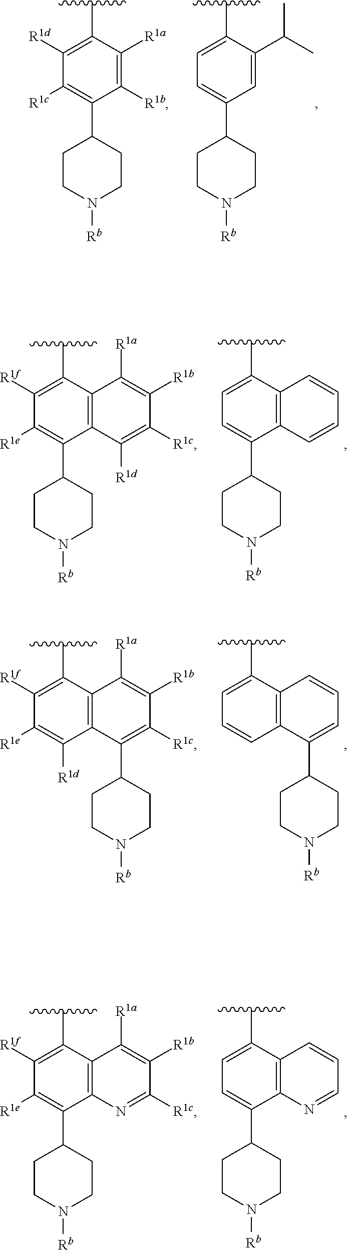

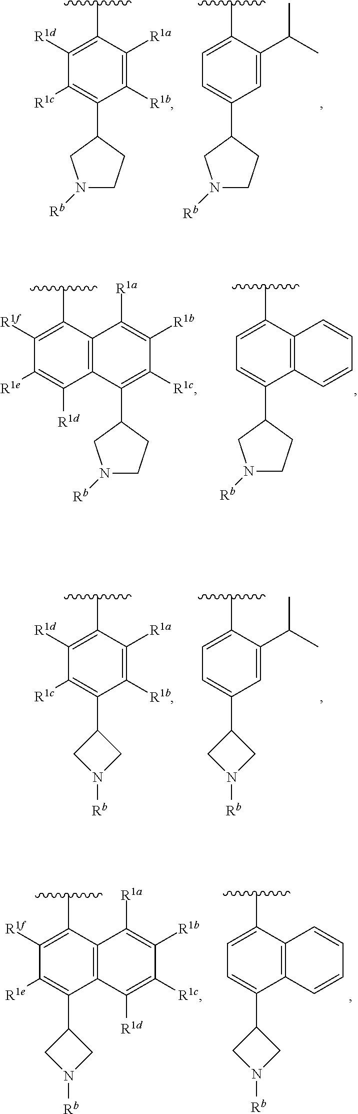

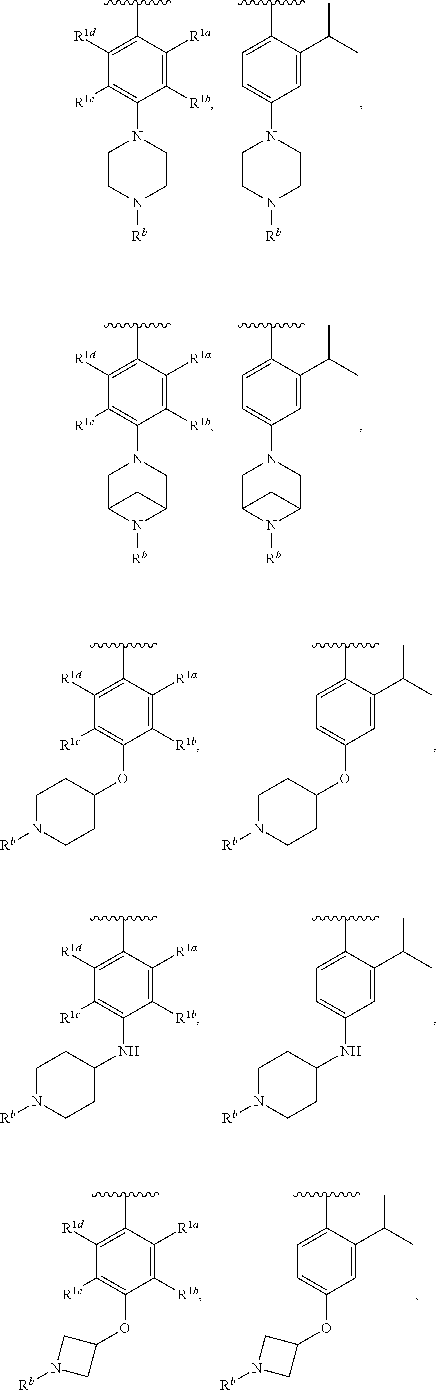

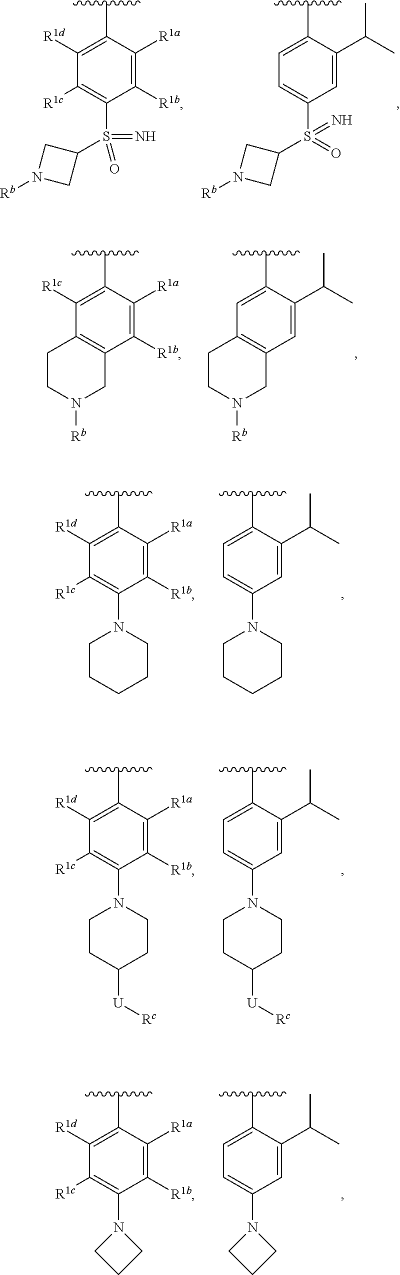

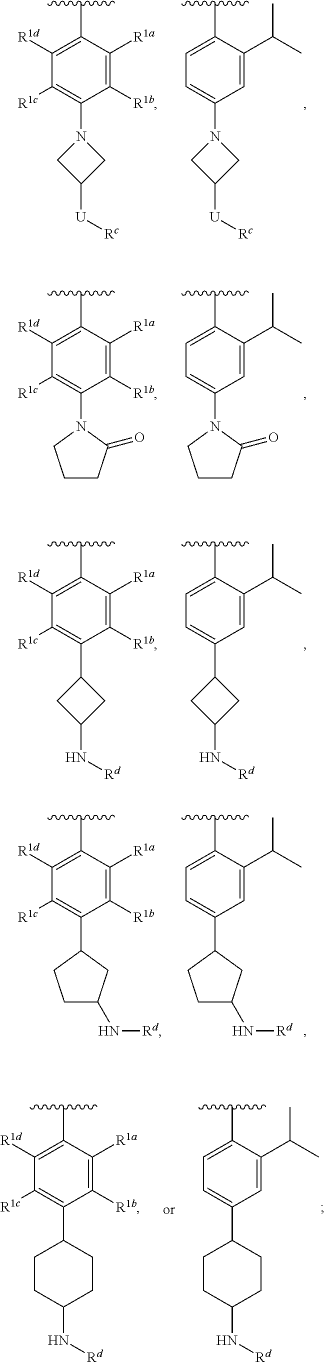

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; U is —O— or —NH—; R^{b} is hydrogen, C₁-C₆ alkyl or 6-membered heteroalkyl; and R^{c} is hydrogen or C₁-C₆ alkyl.

In some embodiments, Ring A-X-Ring B is:

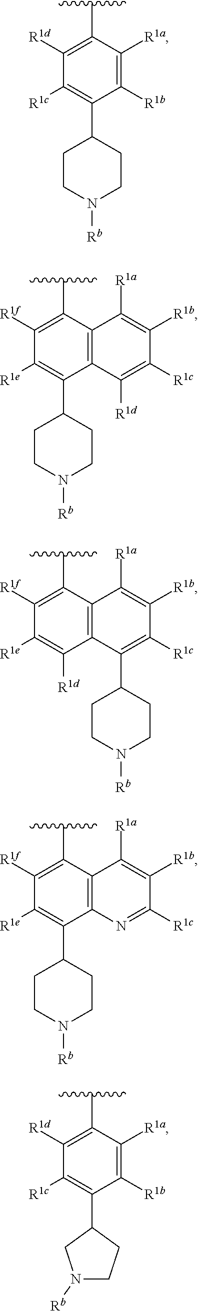

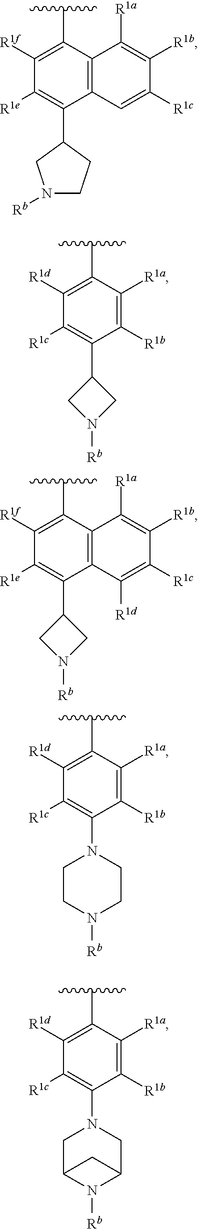

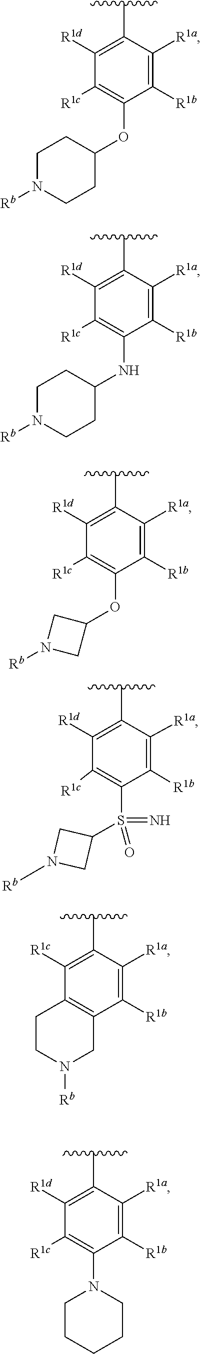

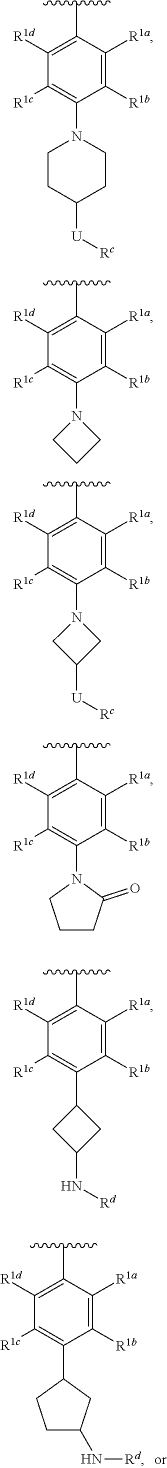

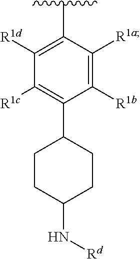

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; U is —O— or —NH—; R^{b} is hydrogen, C₁-C₆ alkyl or 6-membered heteroalkyl; and R^{c} is hydrogen or C₁-C₆ alkyl.

In some embodiments, Ring A-X-Ring B is:

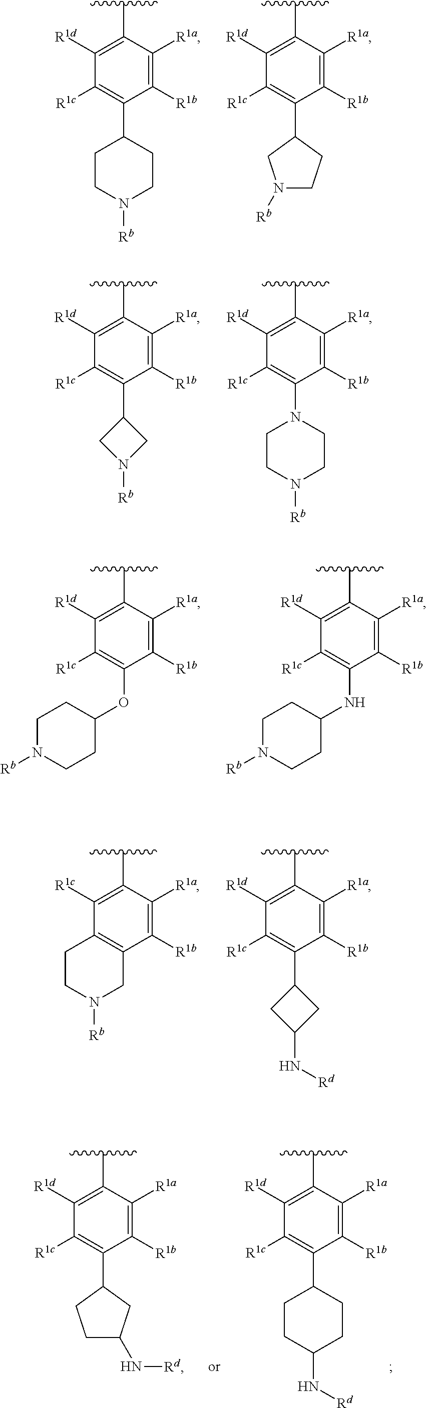

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; Rbis hydrogen, substituted or unsubstituted C₁-C₆ alkyl or substituted or unsubstituted 6-membered heteroalkyl; and R^{c} is hydrogen or C₁-C₆ alkyl; R^{d} is C₁-C₆ alkyl.

In some embodiments, Ring A-X-Ring B is:

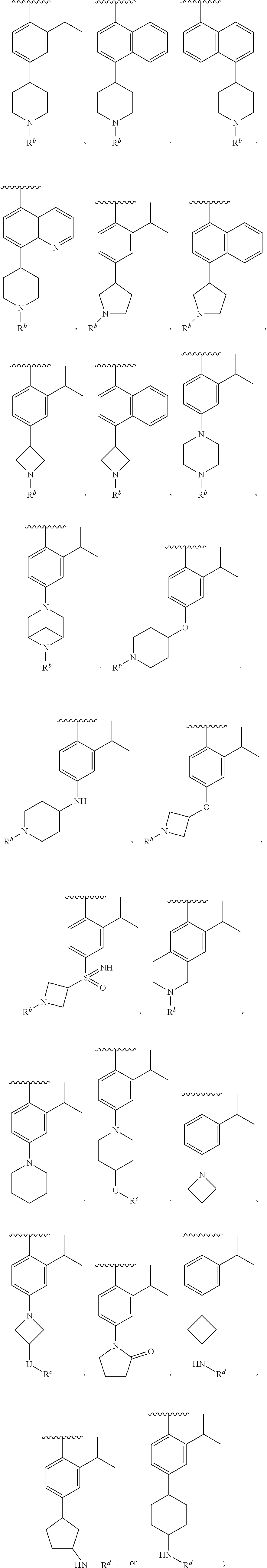

wherein U is —O— or —NH—; R^{b} is hydrogen, C₁-C₆ alkyl or 6-membered heteroalkyl; and R^{c} is hydrogen or C₁-C₆ alkyl.

In some embodiments, Ring A-X-Ring B is:

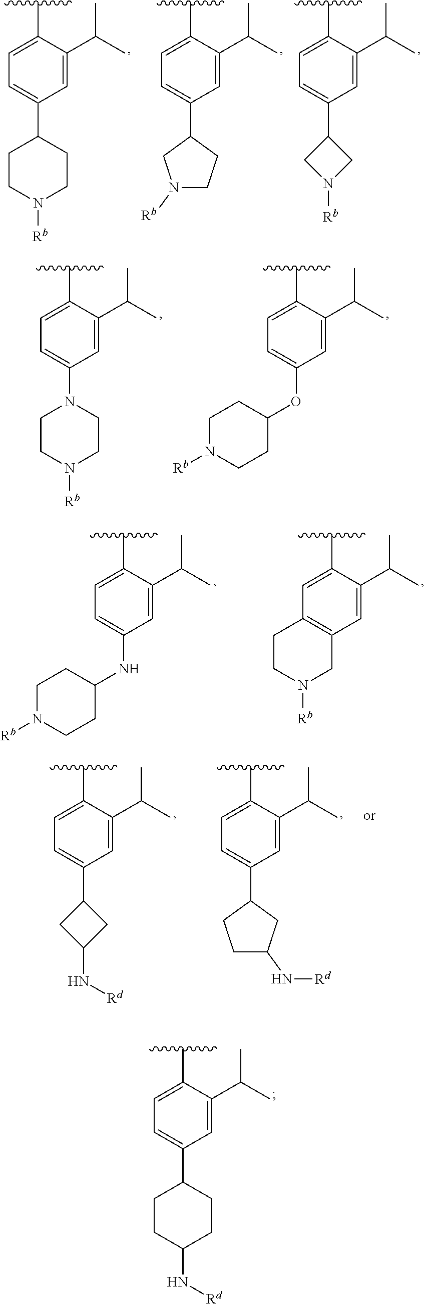

wherein R^{b} is hydrogen, substituted or unsubstituted C₁-C₆ alkyl or substituted or unsubstituted 6-membered heteroalkyl; and R^{d} is C₁-C₆ alkyl.

In some embodiments, Ring A-X-Ring B is:

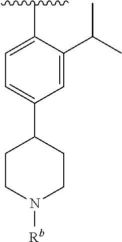

and R^{b} is hydrogen, substituted or unsubstituted C₁-C₆ alkyl or substituted or unsubstituted 6-membered heteroalkyl. In some embodiments, Ring A-X-Ring B is:

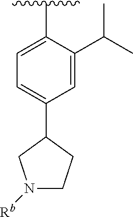

and R^{b} is hydrogen, substituted or unsubstituted C₁-C₆ alkyl or substituted or unsubstituted 6-membered heteroalkyl. In some embodiments, Ring A-X-Ring B is:

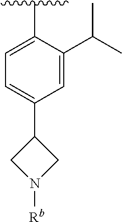

and R^{b} is hydrogen, substituted or unsubstituted C₁-C₆ alkyl or substituted or unsubstituted 6-membered heteroalkyl. In some embodiments, Ring A-X-Ring B is:

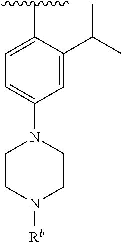

and R^{b} is hydrogen, substituted or unsubstituted C₁-C₆ alkyl or substituted or unsubstituted 6-membered heteroalkyl. In some embodiments, Ring A-X-Ring B is:

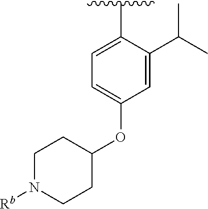

and R^{b} is hydrogen, substituted or unsubstituted C₁-C₆ alkyl or substituted or unsubstituted 6-membered heteroalkyl. In some embodiments, Ring A-X-Ring B is:

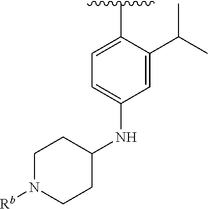

and R^{b} is hydrogen, substituted or unsubstituted C₁-C₆ alkyl or substituted or unsubstituted 6-membered heteroalkyl. In some embodiments, Ring A-X-Ring B is:

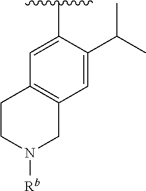

and R^{b} is hydrogen, substituted or unsubstituted C₁-C₆ alkyl or substituted or unsubstituted 6-membered heteroalkyl. In some embodiments, Ring A-X-Ring B is:

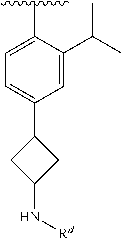

and R^{d} is C₁-C₆ alkyl. In some embodiments, Ring A-X-Ring B is:

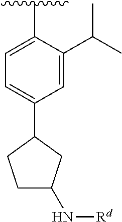

and R^{d} is C₁-C₆ alkyl. In some embodiments, Ring A-X-Ring B is:

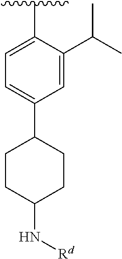

and R^{d} is C₁-C₆ alkyl. In some embodiments, R^{b} is hydrogen. In some embodiments, R^{b} is CH₃.

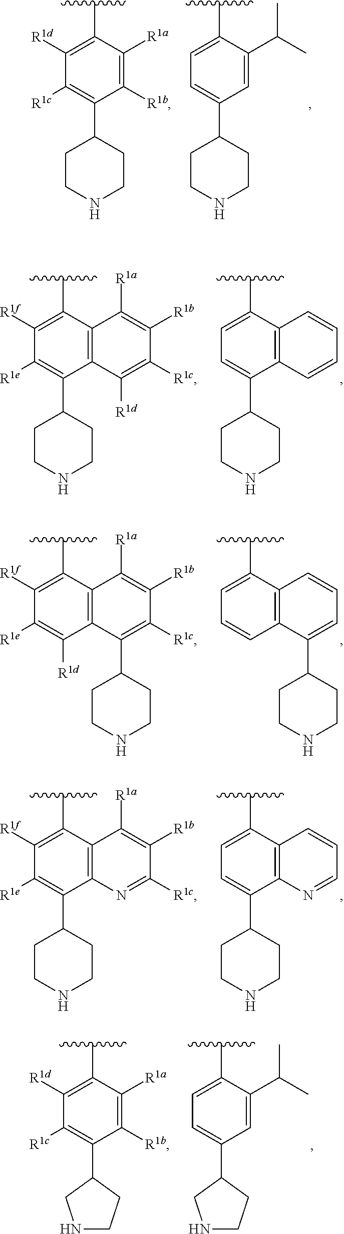

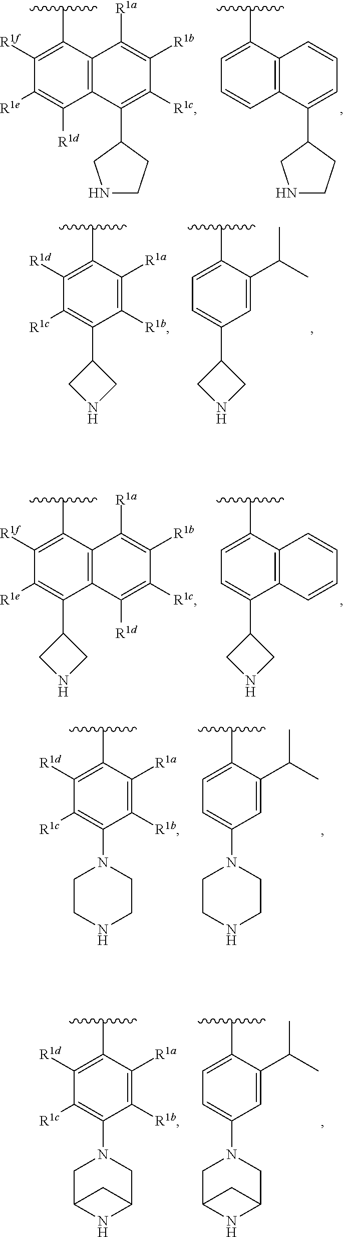

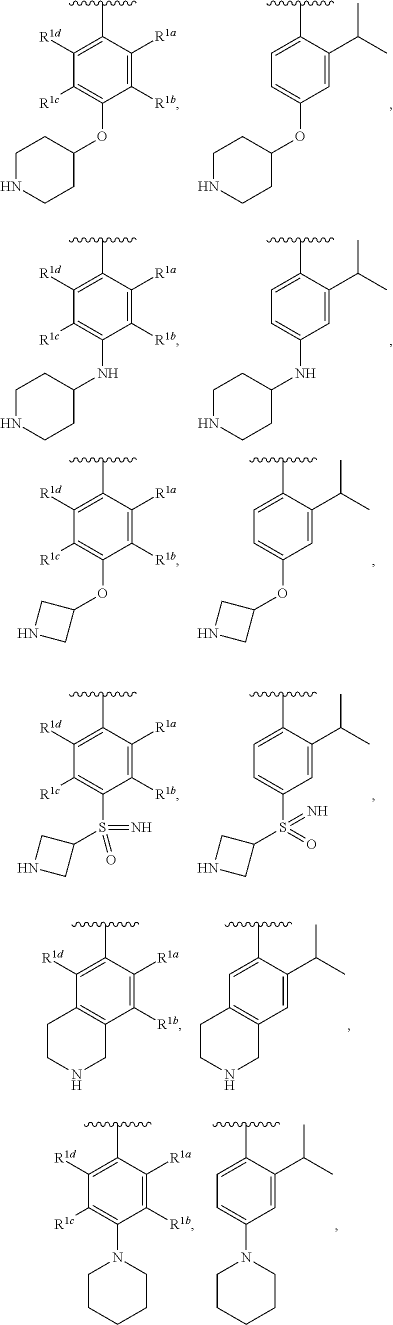

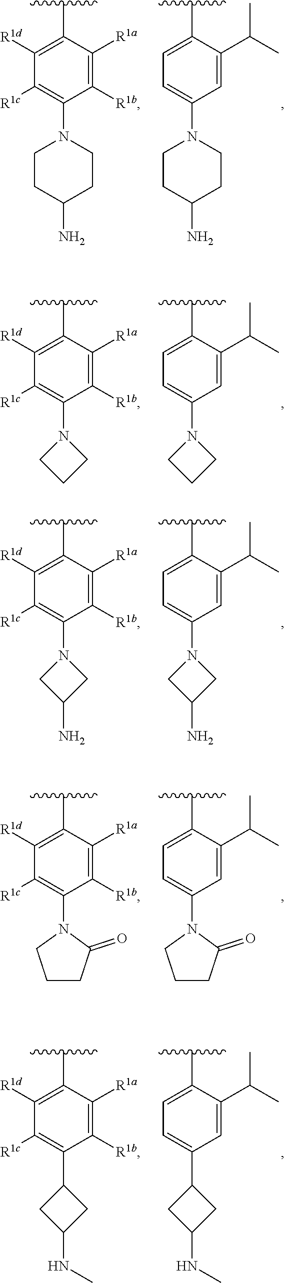

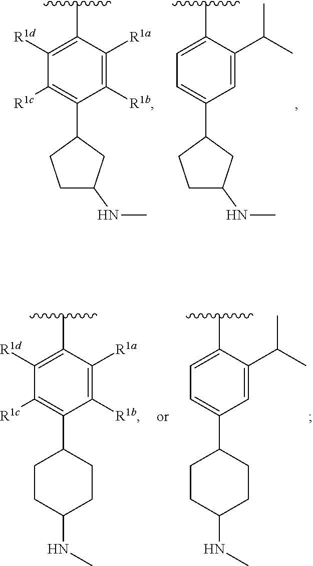

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, -NMCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, Ring A-X-Ring B is:

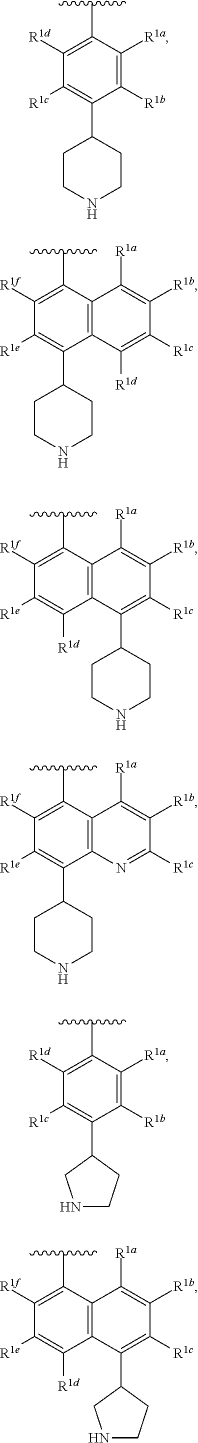

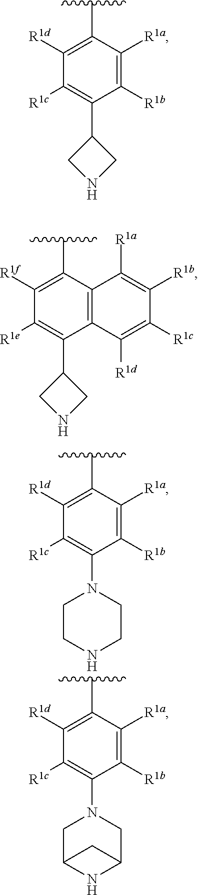

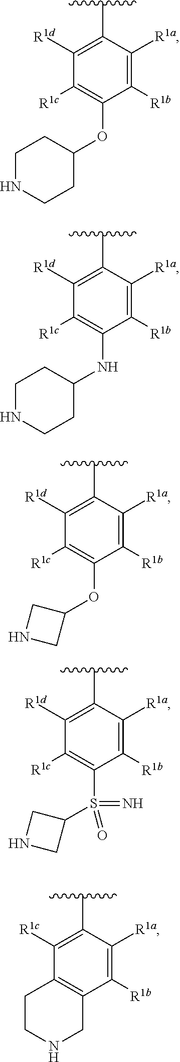

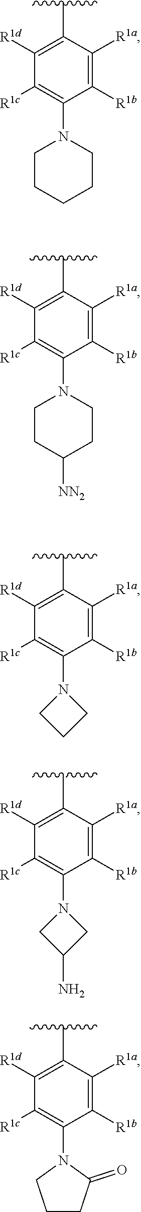

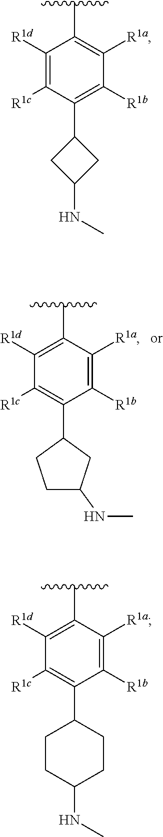

wherein R^{1a},1R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═))OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, Ring A-X-Ring B is:

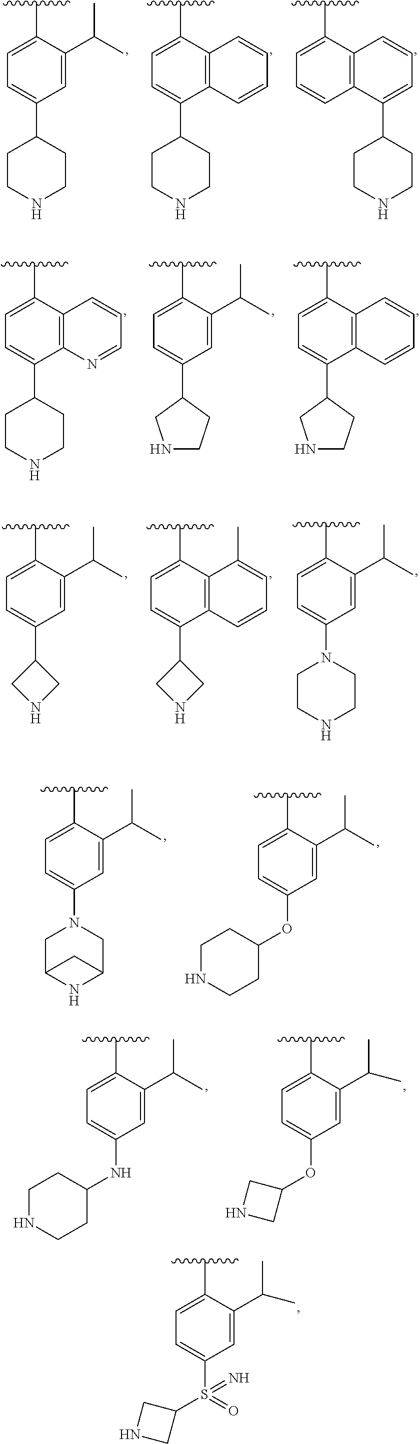

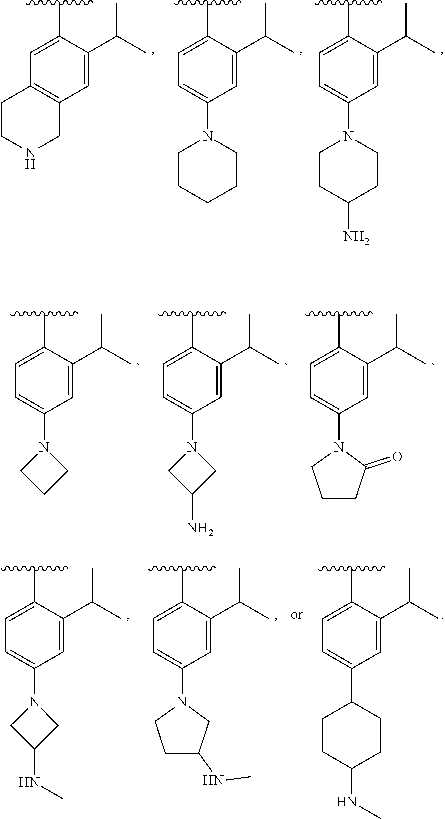

In some embodiments, Ring A-X-Ring B is:

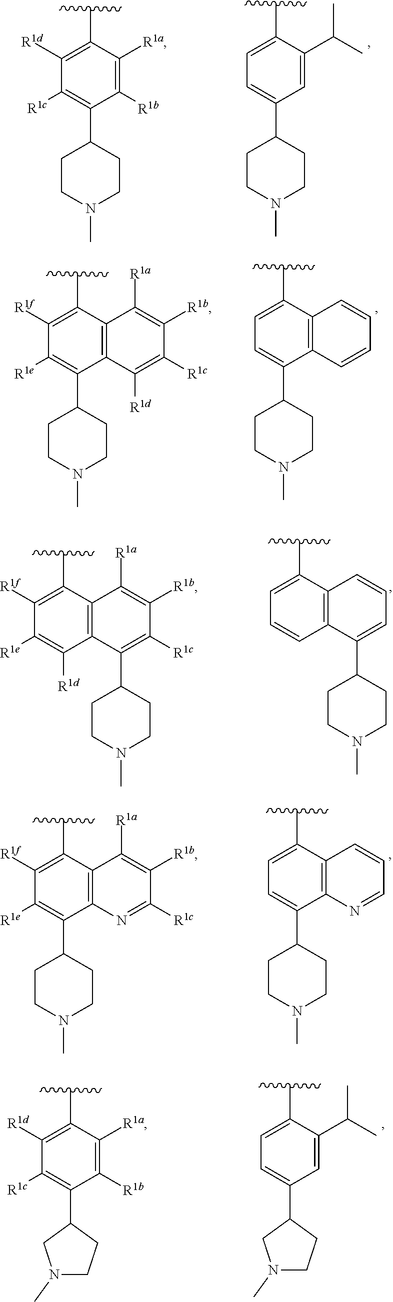

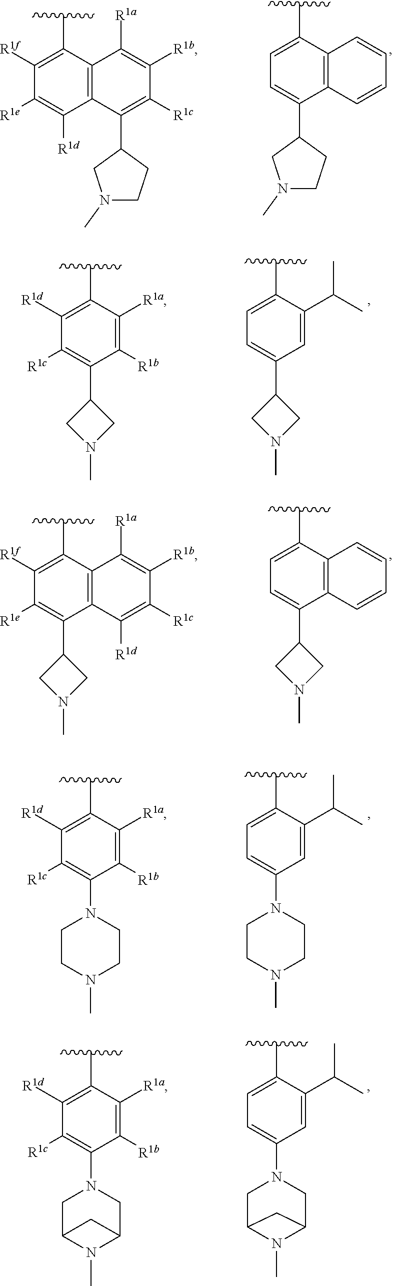

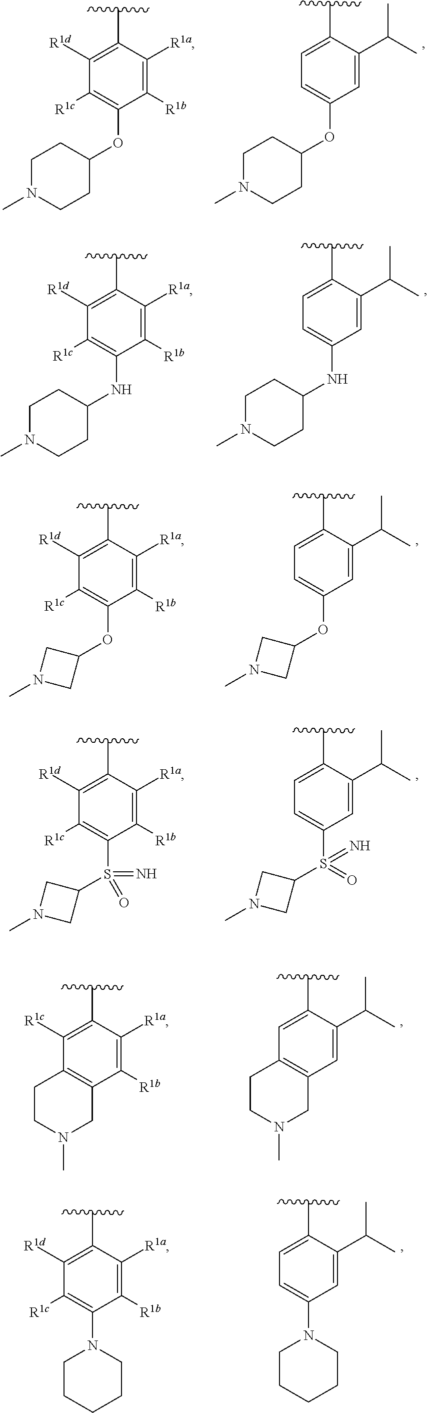

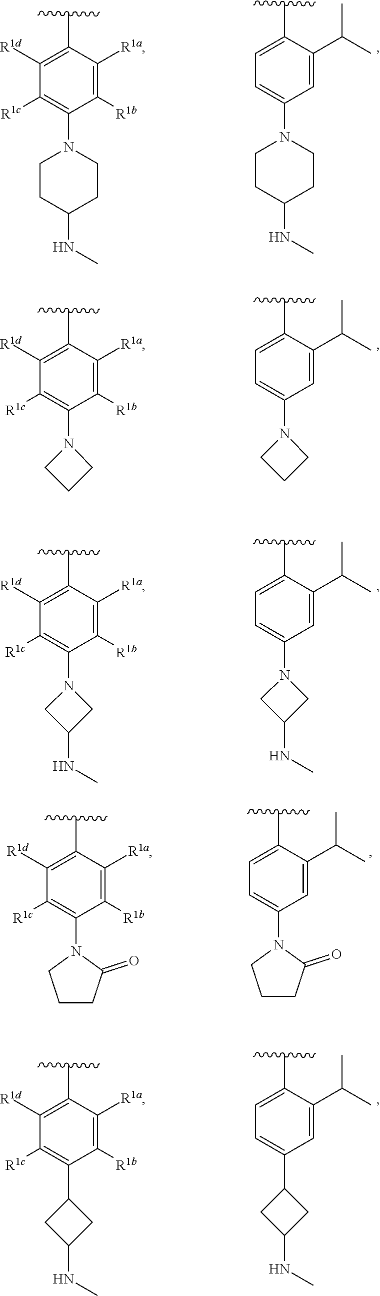

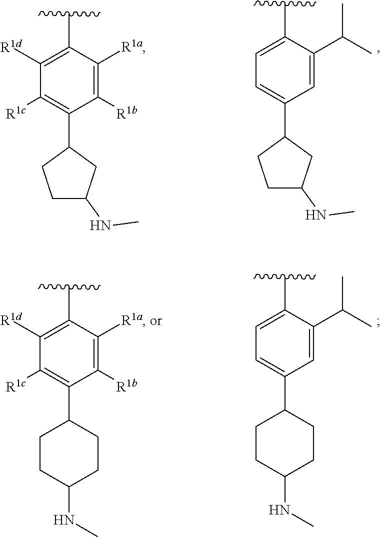

wherein R^{1a}, R^{1b}, R^{1c}, R^{1d}, R^{1e}, and R^{1f} are each independently selected from the group consisting, of hydrogen, —F, —Cl, —Br, —I, —OH, —CF₃, —CN, —CO₂H, —C(═O)CH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, Ring A-X-Ring B is:

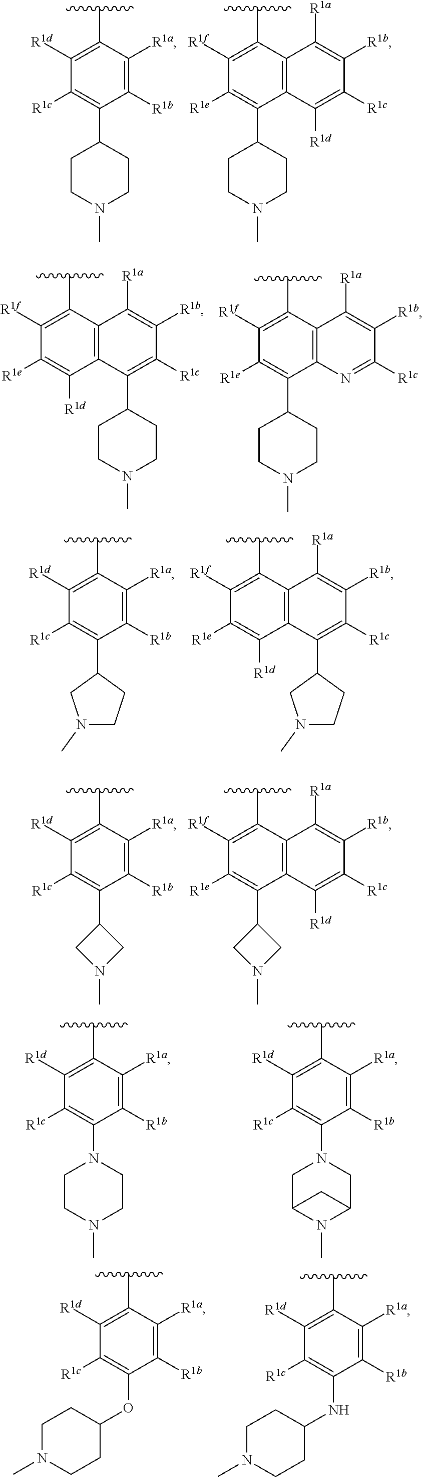

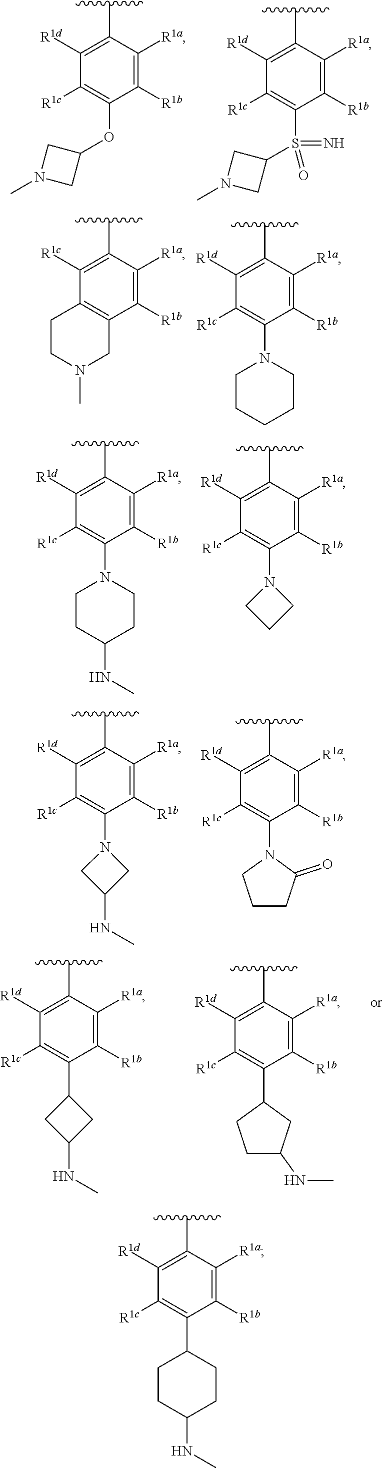

wherein R^{1a}, R^{1b}, R^{1c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

In some embodiments, Ring A-X-Ring B is:

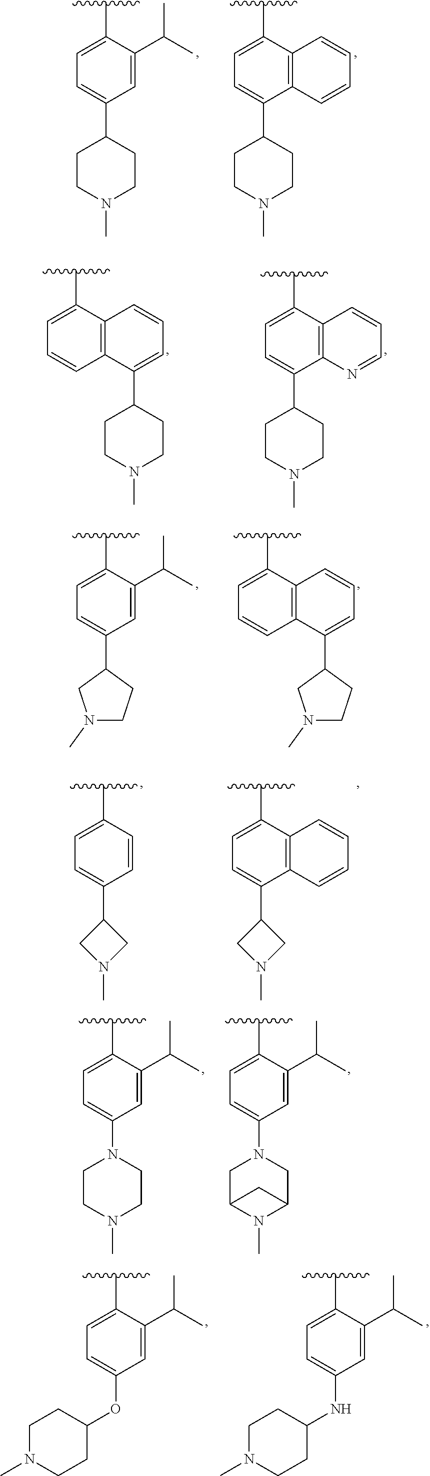

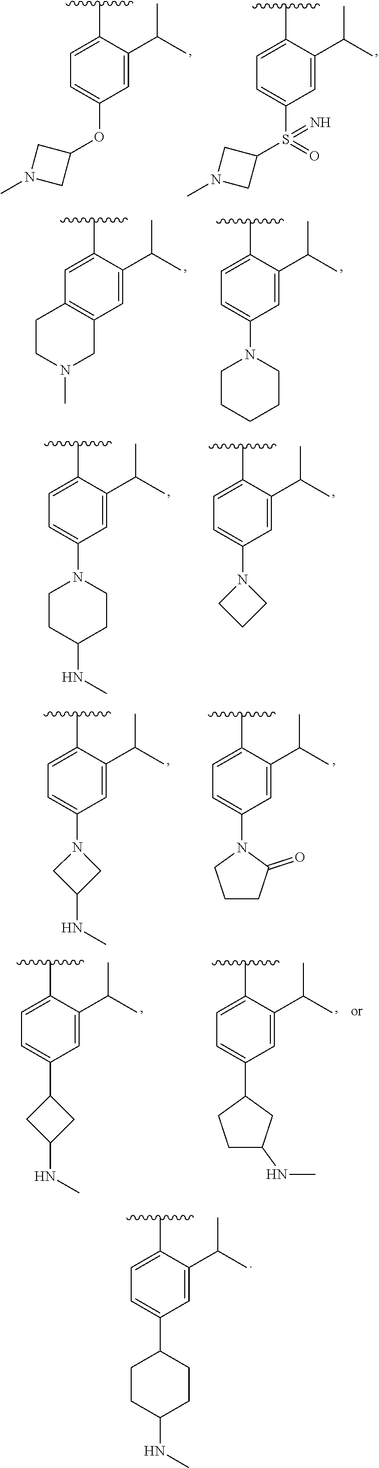

In some embodiments, the compound has the following structure:

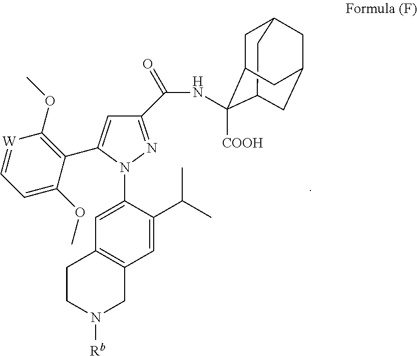

In some embodiments, the compound has the following structure:

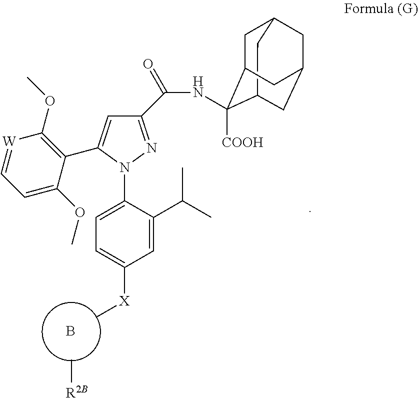

In some embodiments, the compound has the following structure:

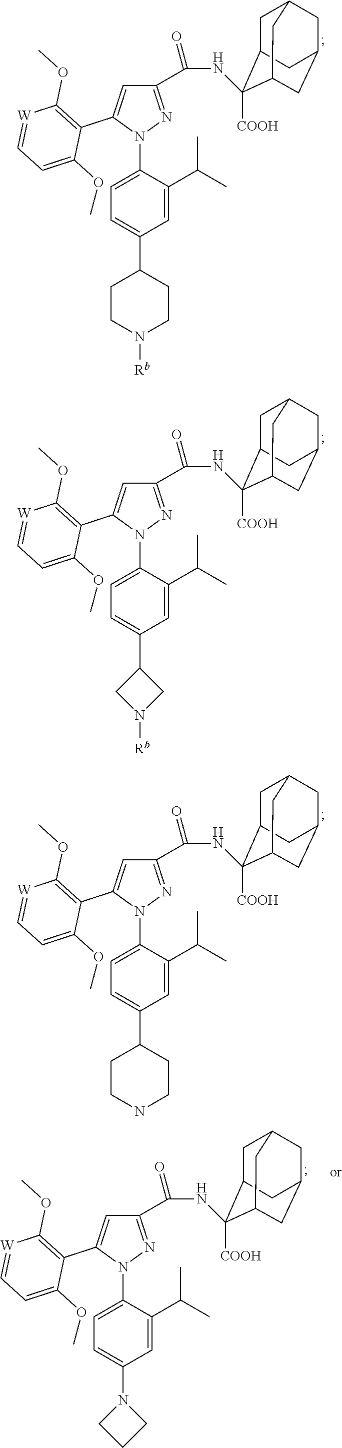

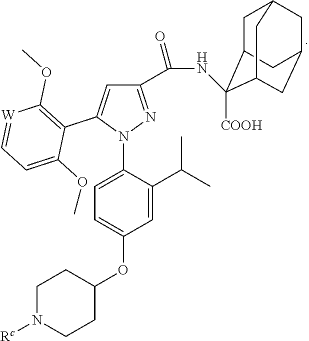

In some embodiments, the compound has the following structure:

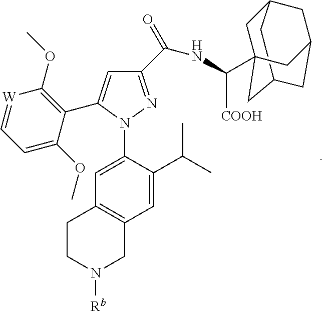

In some embodiments, the compound has the following structure:

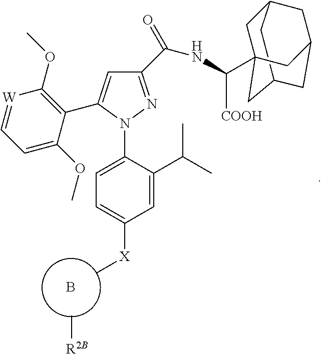

In some embodiments, the compound has following the structure:

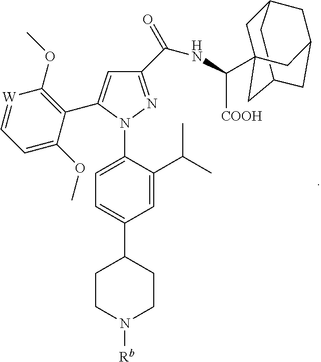

In some embodiments, the compound has the following structure:

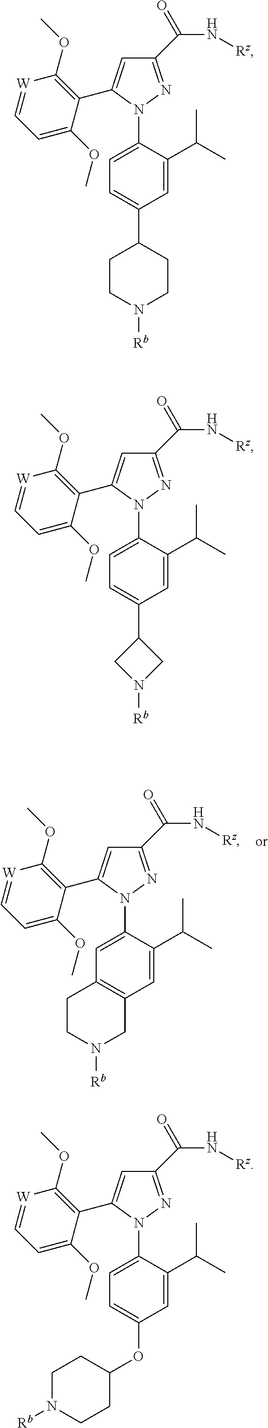

In some embodiments, the compound of Formula (II) has the following structure, or a pharmaceutically acceptable salt thereof:

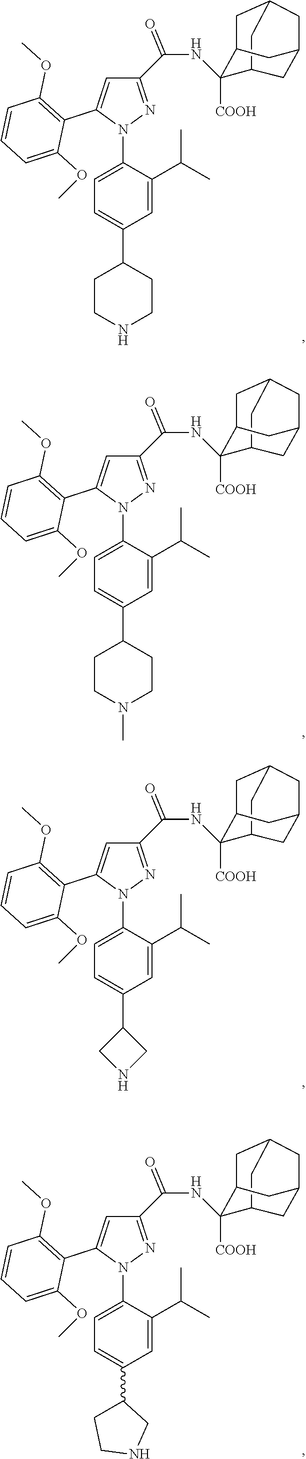 [SMILES: COc1cccc(OC)c1-c1cc(C(=O)NC2(C(=O)O)C3CC4CC(C3)CC2C4)nn1-c1ccc(C2CCNCC2)cc1C(C)C ; COc1cccc(OC)c1-c1cc(C(=O)NC2(C(=O)O)C3CC4CC(C3)CC2C4)nn1-c1ccc(C2CCN(C)CC2)cc1C(C)C ; COc1cccc(OC)c1-c1cc(C(=O)NC2(C(=O)O)C3CC4CC(C3)CC2C4)nn1-c1ccc(C2CNC2)cc1C(C)C ; COc1cccc(OC)c1-c1cc(C(=O)NC2(C(=O)O)C3CC4CC(C3)CC2C4)nn1-c1ccc(C2CCNC2)cc1C(C)C]

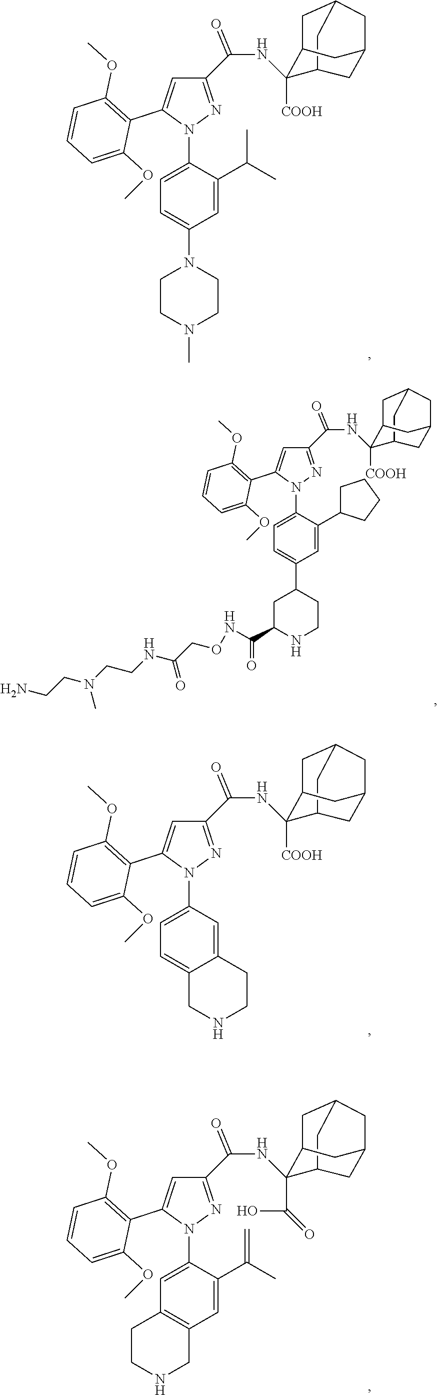 [SMILES: COc1cccc(OC)c1-c1cc(C(=O)NC2(C(=O)O)C3CC4CC(C3)CC2C4)nn1-c1ccc(N2CCN(C)CC2)cc1C(C)C ; COc1cccc(OC)c1-c1cc(C(=O)NC2(C(=O)O)C3CC4CC(C3)CC2C4)nn1-c1ccc(C2CCN[C@@H](C(=O)NOCC(=O)NCCN(C)CCN)C2)cc1C1CCCC1 ; COc1cccc(OC)c1-c1cc(C(=O)NC2(C(=O)O)C3CC4CC(C3)CC2C4)nn1-c1ccc2c(c1)CCNC2 ; C=C(C)c1cc2c(cc1-n1nc(C(=O)NC3(C(=O)O)C4CC5CC(C4)CC3C5)cc1-c1c(OC)cccc1OC)CCNC2]

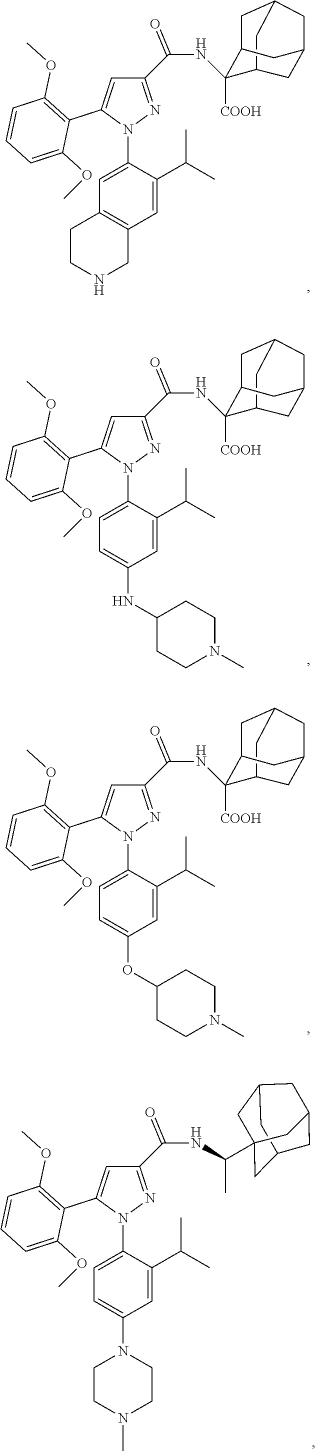 [SMILES: COc1cccc(OC)c1-c1cc(C(=O)NC2(C(=O)O)C3CC4CC(C3)CC2C4)nn1-c1cc2c(cc1C(C)C)CNCC2 ; COc1cccc(OC)c1-c1cc(C(=O)NC2(C(=O)O)C3CC4CC(C3)CC2C4)nn1-c1ccc(NC2CCN(C)CC2)cc1C(C)C ; COc1cccc(OC)c1-c1cc(C(=O)NC2(C(=O)O)C3CC4CC(C3)CC2C4)nn1-c1ccc(OC2CCN(C)CC2)cc1C(C)C ; COc1cccc(OC)c1-c1cc(C(=O)N[C@H](C)C23CC4CC(CC(C4)C2)C3)nn1-c1ccc(N2CCN(C)CC2)cc1C(C)C]

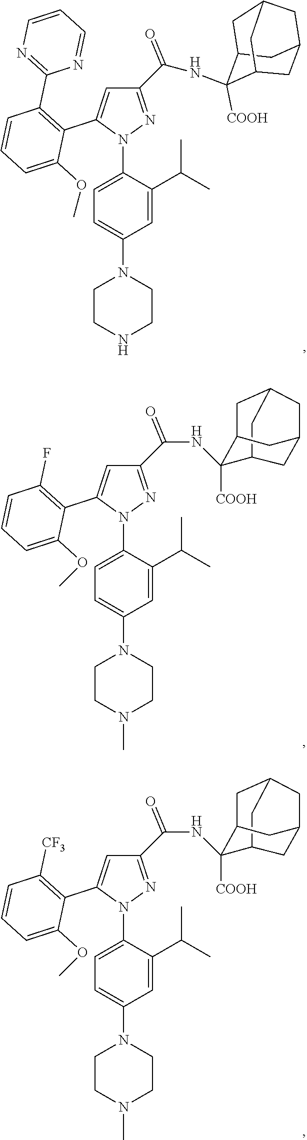 [SMILES: COc1cccc(-c2ncccn2)c1-c1cc(C(=O)NC2(C(=O)O)C3CC4CC(C3)CC2C4)nn1-c1ccc(N2CCNCC2)cc1C(C)C ; COc1cccc(F)c1-c1cc(C(=O)NC2(C(=O)O)C3CC4CC(C3)CC2C4)nn1-c1ccc(N2CCN(C)CC2)cc1C(C)C ; COc1cccc(C(F)(F)F)c1-c1cc(C(=O)NC2(C(=O)O)C3CC4CC(C3)CC2C4)nn1-c1ccc(N2CCN(C)CC2)cc1C(C)C]

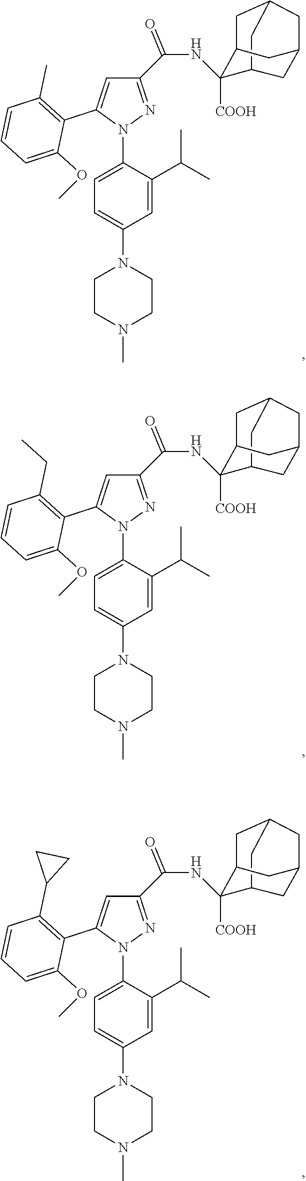 [SMILES: COc1cccc(C)c1-c1cc(C(=O)NC2(C(=O)O)C3CC4CC(C3)CC2C4)nn1-c1ccc(N2CCN(C)CC2)cc1C(C)C ; CCc1cccc(OC)c1-c1cc(C(=O)NC2(C(=O)O)C3CC4CC(C3)CC2C4)nn1-c1ccc(N2CCN(C)CC2)cc1C(C)C ; COc1cccc(C2CC2)c1-c1cc(C(=O)NC2(C(=O)O)C3CC4CC(C3)CC2C4)nn1-c1ccc(N2CCN(C)CC2)cc1C(C)C]

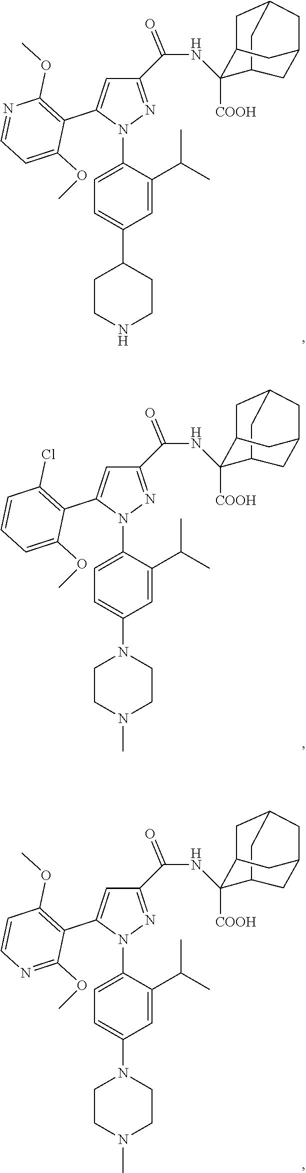 [SMILES: COc1ccnc(OC)c1-c1cc(C(=O)NC2(C(=O)O)C3CC4CC(C3)CC2C4)nn1-c1ccc(C2CCNCC2)cc1C(C)C ; COc1cccc(Cl)c1-c1cc(C(=O)NC2(C(=O)O)C3CC4CC(C3)CC2C4)nn1-c1ccc(N2CCN(C)CC2)cc1C(C)C ; COc1ccnc(OC)c1-c1cc(C(=O)NC2(C(=O)O)C3CC4CC(C3)CC2C4)nn1-c1ccc(N2CCN(C)CC2)cc1C(C)C]

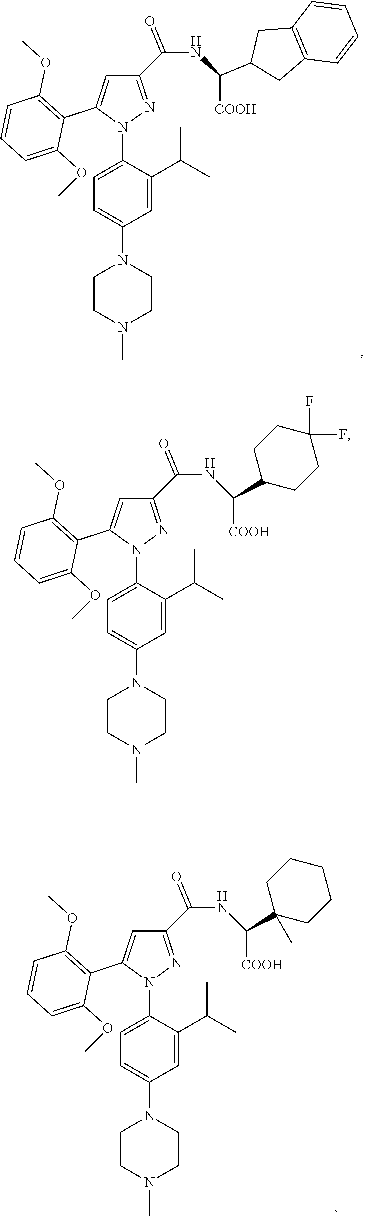 [SMILES: COc1cccc(OC)c1-c1cc(C(=O)N[C@H](C(=O)O)C2Cc3ccccc3C2)nn1-c1ccc(N2CCN(C)CC2)cc1C(C)C ; COc1cccc(OC)c1-c1cc(C(=O)N[C@H](C(=O)O)C2CCC(F)(F)CC2)nn1-c1ccc(N2CCN(C)CC2)cc1C(C)C ; COc1cccc(OC)c1-c1cc(C(=O)N[C@H](C(=O)O)C2(C)CCCCC2)nn1-c1ccc(N2CCN(C)CC2)cc1C(C)C]

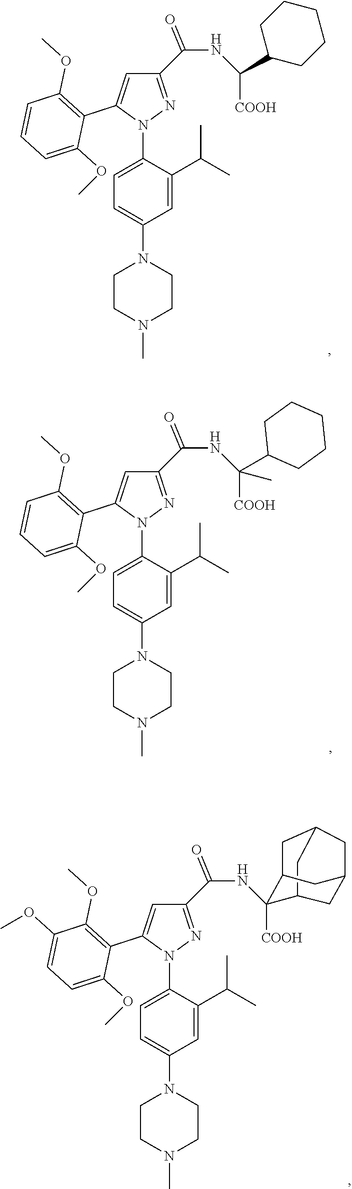 [SMILES: COc1cccc(OC)c1-c1cc(C(=O)N[C@H](C(=O)O)C2CCCCC2)nn1-c1ccc(N2CCN(C)CC2)cc1C(C)C ; COc1cccc(OC)c1-c1cc(C(=O)NC(C)(C(=O)O)C2CCCCC2)nn1-c1ccc(N2CCN(C)CC2)cc1C(C)C ; COc1ccc(OC)c(-c2cc(C(=O)NC3(C(=O)O)C4CC5CC(C4)CC3C5)nn2-c2ccc(N3CCN(C)CC3)cc2C(C)C)c1OC]

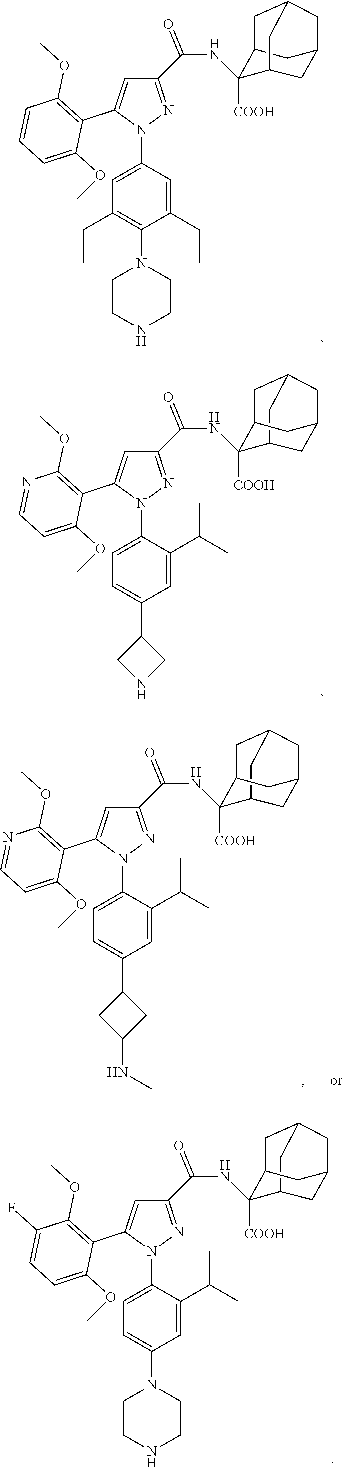 [SMILES: CCc1cc(-n2nc(C(=O)NC3(C(=O)O)C4CC5CC(C4)CC3C5)cc2-c2c(OC)cccc2OC)cc(CC)c1N1CCNCC1 ; COc1ccnc(OC)c1-c1cc(C(=O)NC2(C(=O)O)C3CC4CC(C3)CC2C4)nn1-c1ccc(C2CNC2)cc1C(C)C ; CNC1CC(c2ccc(-n3nc(C(=O)NC4(C(=O)O)C5CC6CC(C5)CC4C6)cc3-c3c(OC)ccnc3OC)c(C(C)C)c2)C1 ; COc1ccc(F)c(OC)c1-c1cc(C(=O)NC2(C(=O)O)C3CC4CC(C3)CC2C4)nn1-c1ccc(N2CCNCC2)cc1C(C)C]

In some embodiments, the compound of Formula (II) is compound 1 or a pharmaceutically acceptable salt thereof; compound 2 or a pharmaceutically acceptable salt thereof; compound 3 or a pharmaceutically acceptable salt thereof; compound 4 or a pharmaceutically acceptable salt thereof; compound 5 or a pharmaceutically acceptable salt thereof; compound 6 or a pharmaceutically acceptable salt thereof; compound 7 or a pharmaceutically acceptable salt thereof; compound 8 or a pharmaceutically acceptable salt thereof; compound 9 or a pharmaceutically acceptable salt thereof; compound 10 or a pharmaceutically acceptable salt thereof; compound 11 or a pharmaceutically acceptable salt thereof; compound 12 or a pharmaceutically acceptable salt thereof; compound 13 or a pharmaceutically acceptable salt thereof; compound 14 or a pharmaceutically acceptable salt thereof; compound 76 or a pharmaceutically acceptable salt thereof; compound 77 or a pharmaceutically acceptable salt thereof; compound 78 or a pharmaceutically acceptable salt thereof; compound 79 or a pharmaceutically acceptable salt thereof; compound 80 or a pharmaceutically acceptable salt thereof; compound 81 or a pharmaceutically acceptable salt thereof; compound 82 or a pharmaceutically acceptable salt thereof; compound 83 or a pharmaceutically acceptable salt thereof; compound 125 or a pharmaceutically acceptable salt thereof; compound 126 or a pharmaceutically acceptable salt thereof; compound 127 or a pharmaceutically acceptable salt thereof; compound 128 or a pharmaceutically acceptable salt thereof; compound 129 or a pharmaceutically acceptable salt thereof; compound 130 or a pharmaceutically acceptable salt thereof; compound 131 or a pharmaceutically acceptable salt thereof; compound 132 or a pharmaceutically acceptable salt thereof; compound 133 or a pharmaceutically acceptable salt thereof; compound 134 or a pharmaceutically acceptable salt thereof; compound 135 or a pharmaceutically acceptable salt thereof; or compound 136 or a pharmaceutically acceptable salt thereof.

In some embodiments, the compound of Formula (II) is compound 1 or a pharmaceutically acceptable salt thereof. In some embodiments, the compound of Formula (II) is compound 2 or a pharmaceutically acceptable salt thereof. In some embodiments, the compound of Formula (II) is compound 3 or a pharmaceutically acceptable salt thereof. In some embodiments, the compound of Formula (II) is compound 4 or a pharmaceutically acceptable salt thereof. In some embodiments, the compound of Formula (II) is compound 5 or a pharmaceutically acceptable salt thereof. In some embodiments, the compound of Formula (II) is compound 6 or a pharmaceutically acceptable salt thereof. In some embodiments, the compound of Formula (II) is compound 7 or a pharmaceutically acceptable salt thereof. In some embodiments, the compound of Formula (II) is compound 8 or a pharmaceutically acceptable salt thereof. In some embodiments, the compound of Formula (II) is compound 9 or a pharmaceutically acceptable salt thereof. In some embodiments, the compound of Formula (II) is compound 10 or a pharmaceutically acceptable salt thereof. In some embodiments, the compound of Formula (II) is compound 11 or a pharmaceutically acceptable salt thereof. In some embodiments, the compound of Formula (II) is compound 12 or a pharmaceutically acceptable salt thereof. In some embodiments, the compound of Formula (II) is compound 13 or a pharmaceutically acceptable salt thereof. In some embodiments, the compound of Formula (II) is compound 14 or a pharmaceutically acceptable salt thereof. In some embodiments, the compound of Formula (II) is compound 76 or a pharmaceutically acceptable salt thereof. In some embodiments, the compound of Formula (II) is compound 77 or a pharmaceutically acceptable salt thereof. In some embodiments, the compound of Formula (II) is compound 78 or a pharmaceutically acceptable salt thereof. In some embodiments, the compound of Formula (II) is compound 79 or a pharmaceutically acceptable salt thereof. In some embodiments, the compound of Formula (II) is compound 80 or a pharmaceutically acceptable salt thereof. In some embodiments, the compound of Formula (II) is compound 81 or a pharmaceutically acceptable salt thereof. In some embodiments, the compound of Formula (II) is compound 82 or a pharmaceutically acceptable salt thereof. In some embodiments, the compound of Formula (II) is compound 83 or a pharmaceutically acceptable salt thereof. In some embodiments, the compound of Formula (II) is compound 125 or a pharmaceutically acceptable salt thereof. In some embodiments, the compound of Formula (II) is compound 126 or a pharmaceutically acceptable salt thereof. In some embodiments, the compound of Formula (H) is compound 127 or a pharmaceutically acceptable salt thereof. In some embodiments, the compound of Formula (II) is compound 128 or a pharmaceutically acceptable salt thereof. In some embodiments, the compound of Formula (II) is compound 129 or a pharmaceutically acceptable salt thereof. In some embodiments, the compound of Formula (II) is compound 130 or a pharmaceutically acceptable salt thereof. In some embodiments, the compound of Formula (II) is compound 131 or a pharmaceutically acceptable salt thereof. In some embodiments, the compound of Formula (II) is compound 132 or a pharmaceutically acceptable salt thereof. In some embodiments, the compound of Formula (II) is compound 133 or a pharmaceutically acceptable salt thereof. In some embodiments, the compound of Formula (II) is compound 134 or a pharmaceutically acceptable salt thereof. In some embodiments, the compound of Formula (II) is compound 135 or a pharmaceutically acceptable salt thereof. In some embodiments, the compound of Formula (II) is compound 136 or a pharmaceutically acceptable salt thereof.

### Further Forms of Compounds

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. See for example Handbook of Pharmaceutical Salts: Properties, Selection and Use; International Union of Pure and Applied Chemistry, Wiley-VCH 2002. S. M. Berge, L. D. Bighley, D. C. Monkhouse, J. Pharm. Sci. 1977, 66, 1-19; and P. H. Stahl and C. G. Wermuth, editors, Handbook of Pharmaceutical Salts: Properties, Selection and Use, Weinheim/Zürich:Wiley-VCH/VHCA, 2002; which are incorporated herein by reference. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (A) or Formula (I) with an acid. In some embodiments, the acid is an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to: 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (A) or Formula (I), is prepared as a chloride salt, sulfate salt, bromide salt, mesylate salt, maleate salt, citrate salt or phosphate salt.

In some embodiments, pharmaceutically acceptable salts are obtained by reacting a compound of Formula (A) or Formula (I), with a base. In some cases, compounds described herein coordinate with an organic base, such as, but not limited to, ethanolamine, diethanolamine, triethanolamine, tromethamine, meglumine, N-methylglucamine, dicyclohexylamine, or tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non -stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, any one of the hydrogen atoms on the organic radicals (e.g., alkyl groups, aromatic rings) of compounds described herein are replaced with deuterium.

In some embodiments, the compounds of Formula (A) or Formula (I), possess one or more stereocenters and each stereocenter exists independently in either the R or S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds described herein, are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the formation of diastereomeric salts and separation by recrystallization, or chromatography, or any combination thereof. See for example Jean Jacques, Andre Collet, Samuel H. Wilen, “Enantiomers, Racemates and Resolutions”, John Wiley And Sons, Inc., 1981, which is incorporated herein by reference. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds described herein are synthesized using standard synthetic techniques or using methods known in the art in combination with methods described herein.

Unless otherwise indicated, conventional methods of mass spectroscopy, NMR, and HPLC are employed.

Compounds are prepared using standard organic chemistry techniques such as those described in, for example, March's Advanced Organic Chemistry, 6^{th} Edition, John Wiley and Sons, Inc. Compounds may also be prepared using solid-phase peptide synthesis techniques such as those described in, for example, Solid Phase Peptide Synthesis, 2^{nd} Edition, The Pierce Chemical Co., Rockford, Ill. (1984). Alternative reaction conditions for the synthetic transformations described herein may be employed such as variation of solvent, reaction temperature, reaction time, as well as different chemical reagents and other reaction conditions.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to, delivery via parenteral routes (including injection or infusion, and subcutaneous).

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and contain optional agents as excipients such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (A) or Formula (I), or a pharmaceutically acceptable salt or solvate thereof. In some embodiments, the compound of Formula (A) or Formula (I) or pharmaceutically acceptable salt or solvate thereof is administered in a pharmaceutical composition. In some embodiments, the subject has cancer. In some embodiments, the cancer is a solid tumor. In some embodiments, the subject has a noncancerous tumor. In some embodiments, the subject has an adenoma.

In some embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (A) or Formula (I), or a pharmaceutically acceptable salt or solvate thereof. In some embodiments, the compound of Formula (A) or Formula (I), or pharmaceutically acceptable salt or solvate thereof releases a number of alpha particles by natural radioactive decay. In some embodiments, the released alpha particles are sufficient to kill the tumor cell. In some embodiments, the released alpha particles are sufficient to stop cell growth. In some embodiments, the tumor cell is a malignant tumor cell. In some embodiments, the tumor cell is a benign tumor cell. In some embodiments, the method comprises killing a tumor cell with a beta-particle emitting radionuclide. In some embodiments, the method comprises killing a tumor cell with an alpha-particle emitting radionuclide. In some embodiments, the method comprises killing a tumor cell with a gamma-particle emitting radionuclide.

In one aspect, provided herein are methods and compositions for treating cancers. In some embodiments, the cancer is lung cancer. In some embodiments, the cancer is pancreatic cancer. In some embodiments, the cancer is colorectal cancer. In some embodiments, the cancer is head and neck cancers. In some embodiments, the cancer is breast cancer. In some embodiments, the cancer is prostate cancer. In some embodiments, the lung cancer is non-small cell lung cancer.

In one aspect, provided herein are methods and compositions for treating an adenoma.

In one aspect, provided herein are methods and compositions for treating a carcinoma.

In one aspect, provided herein is a method for identifying tissues or organs in a mammal that overexpress NTSR1 comprising: (i) administering to the mammal a NTSR1 radiopharmaceutical described herein, or a pharmaceutically acceptable salt thereof; and (ii) performing single-photon emission computerized tomography (SPECT) or positron emission tomography (PET) analysis on the mammal. In some embodiments, the method comprises: (i) administering to the mammal a NTSR1 radiopharmaceutical described herein, or a pharmaceutically acceptable salt thereof; and (ii) performing positron emission tomography (PET) analysis on the mammal.

In some embodiments, the mammal was diagnosed with cancer. In some embodiments, the mammal was diagnosed with ovarian cancer. In some embodiments, the mammal was diagnosed with breast cancer. In some embodiments, the mammal was diagnosed with endometrial cancer. In some embodiments, the mammal was diagnosed with prostate cancer. In some embodiments, the tissues in the mammal that overexpress NTSR1 are tumors.

In some embodiments, a NTSR1 radiopharmaceutical described herein, or a pharmaceutically acceptable salt thereof are used in a method for the in vivo imaging of a subject. In some embodiments, the method includes the steps of: (i) administering to the mammal NTSR1 radiopharmaceutical described herein, or a pharmaceutically acceptable salt thereof; (ii) waiting a sufficient amount of time to allow the NTSR1 radiopharmaceutical, to accumulate at a tissue or cell site to be imaged; and (iii) imaging the cells or tissues with a non-invasive imaging technique.

In some embodiments, the non-invasive imaging technique is single-photon emission computerized tomography (SPECT) or positron emission tomography (PET) analysis. In some embodiments, the non-invasive imaging technique is single-photon emission computerized tomography (SPECT). In some embodiments, the non-invasive imaging technique is selected from positron emission tomography imaging, or positron emission tomography with computed tomography imaging, and positron emission tomography with magnetic resonance imaging.

### Methods of Dosing and Treatment Regimens

In one embodiment, the NTSR1 radiopharmaceutical described herein, or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (A) or Formula (I), or a pharmaceutically acceptable salt thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans.

The amount of a compound of Formula (A) or Formula (I), or pharmaceutically acceptable salts thereof, that are administered are sufficient to deliver a therapeutically effective dose to the particular subject. In some embodiments, dosages of a compound of Formula (A) or Formula (I), are between about 0.1 μg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of a compound of Formula (A) or Formula (I), or a pharmaceutically acceptable salt thereof described herein for methods of treating a disease as described herein is about 0.001 mg/kg to about 1 mg/kg body weight of the subject per dose. In some embodiments, the dose is about 0.001 mg to about 1000 mg per dose for the subject being treated. In some embodiments, a compound of Formula (A) or Formula (I), or a pharmaceutically acceptable salt thereof described herein is administered to a subject at a dosage of from about 0.01 mg to about 500 mg, from about 0.01 mg to about 100 mg, or from about 0.01 mg to about 50 mg.

In some embodiments, a compound of Formula (A) or Formula (I), or a pharmaceutically acceptable salt thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, a compound of Formula (A) or Formula (I), or a pharmaceutically acceptable salt thereof described herein is administered to a subject at a dosage of about 0.01 Gbq to about 1000 Gbq, about 0.5 Gbq to about 100 Gbq, or about 1 Gbq to about 50 Gbq.

In some embodiments, the dose is administered once a day, 1 to 3 times a week, 1 to 4 times a month, or 1 to 12 times a year.

In any of the aforementioned aspects are further embodiments in which the effective amount of the NTSR1 radiopharmaceutical described herein, or a pharmaceutically acceptable salt thereof, is: (a) systemically administered to the mammal; and/or (b) intravenously administered to the mammal; and/or (c) administered by injection to the mammal.

In certain instances, it is appropriate to administer at least one NTSR1 radiopharmaceutical described herein, or a pharmaceutically acceptable salt thereof, in combination with one or more other therapeutic agents.

### Certain Terminology

Unless otherwise stated, the following terms used in this application have the definitions given below. The use of the term “including” as well as other forms, such as “include,” “includes,” and “included,” is not limiting. The section headings used herein are for organizational purposes only and are not to be construed as limiting the subject matter described.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e., groups containing 1 carbon atom, 2 carbon atoms, 3 carbon atoms 4 carbon atoms, 5 carbon atoms or 6 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e., a —C₁-C₁₀ alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a —C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, the alkyl group is an “alkenyl” or “alkynyl” group.

An “alkylene” group refers to a divalent alkyl radical. Any of the above-mentioned monovalent alkyl groups may be an alkylene by abstraction of a second hydrogen atom from the alkyl. In some embodiments, an alkylene is a —C₁-C₆ alkylene. In other embodiments, an alkylene is a —C₁-C₄ alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—. In some embodiments, an alkylene is —CH₂Cl₂—.

An “alkoxy” group refers to an (alkyl)O— group, where alkyl is as defined herein.

The term “alkenyl” refers to a type of alkyl group in which at least one carbon-carbon double bond is present. In one embodiment, an alkenyl group has the formula: —C(R)═CR₂, wherein R refers to the remaining portions of the alkenyl group, which may be the same or different. In some embodiments, each R is independently H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portion of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH₃, and —CH₂C≡CH.

The term “heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. In some embodiments, the “heteroalkyl” group has 2 to 10 atoms in the backbone, which include a combination of carbon atoms and heteroatoms (e.g. N, O, S), i.e., a 2 to 10-membered heteroalkyl. In some embodiments, the heteroalkyl is attached to the rest of the molecule at a carbon atom of the heteroalkyl. In one embodiment, a heteroalkyl is a 2 to 8 membered heteroalkyl.

A “heteroalkylene” group refers to a divalent alkyl radical derived from heteroalkyl, as exemplified, but not limited by, —CH₂—CH₂—O—CH₂—CH₂— and —CH₂—O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(═O)O— represents both —C(═O)O— and —OC(═O)—. Additionally, the formula —C(═O)NH-represents both —C(═O)NH— and —NHC(═O)—.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom that is different from carbon. In some embodiments, carbocycles are monocyclic, bicyclic, or multicyclic. In some embodiments, a carbocycle is a monocyclic carbocycle or a bicyclic carbocycle. A bicyclic carbocycle is a fused bicyclic carbocycle, bridged bicyclic carbocycle, or spirocyclic bicyclic carbocycle. A bicyclic carbocycle is a fused bicyclic carbocycle, bridged bicyclic carbocycle, or spirocyclic bicyclic carbocycle. The term “fused bicyclic carbocycle” refers to two carbocycles that are joined together and share two adjacent carbon atoms. The term “bridged bicyclic carbocycles” refers to two carbocycles that are joined together and share three or more carbon atoms. The term “spirocyclic carbocycles” or a “spiro bicyclic carbocycles” refers to two carbocycles that are joined together and share a single carbon atom.

A carbocycle is fully saturated (i.e., no units of unsaturation, also known as cycloalkyl), partially unsaturated (i.e., at least one unit of unsaturation, i.e., cycloalkenyl), or fully unsaturated (i.e., aryl). Carbocycles include aryls and cycloalkyls.

In some embodiments, at least one of the two rings of a partially unsaturated bicyclic carbocycle is aromatic. In some embodiments, both rings of a bicyclic carbocycle are aromatic. In some embodiments, partially unsaturated bicyclic carbocycles include indanyl

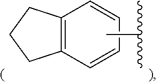

indenyl

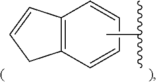

and tetrahydronaphthyl

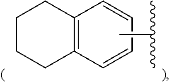

1,4-dihydronaphthalene, and 1,2-dihydronaphthalene. In some other embodiments, partially unsaturated bicyclic carbocycles include fused bicyclic cycloalkenyl, spirocyclic cycloalkenyl and bridged bicyclic cycloalkenyl.

As used herein, the term “aryl” refers to an aromatic ring wherein each of the atoms forming the ring is a carbon atom. In one aspect, aryl is phenyl

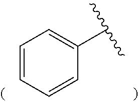 [SMILES: *c1ccccc1]

or a naphthyl

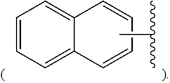

monocyclic aryl is phenyl. A bicyclic aryl is naphthyl. In some embodiments, an aryl is a C₆-C₁₀ aryl. Depending on the structure, an aryl group is a monoradical or a diradical (i.e., an arylene group).

The term “cycloalkyl” refers to a monocyclic, bicyclic, or polycyclic aliphatic, non-aromatic radical, wherein each of the atoms forming the ring (i.e., skeletal atoms) is a carbon atom. In some embodiments, a cycloalkyl is a fused cycloalkyl, spirocyclic cycloalkyl, or bridged cycloalkyl. Cycloalkyl groups include groups having from 3 to 12 ring atoms. In some embodiments, cycloalkyl groups are selected from cyclopropyl, cyclobutyl, cyclopentyl, cyclopentenyl, cyclohexyl, cyclohexenyl, cycloheptyl, cyclooctyl, spiro[2.2]pentyl, and bicyclo[1.1.1.]pentyl. In some embodiments, a cycloalkyl is a C₃-C₆ cycloalkyl. In some embodiments, a cycloalkyl is a C₃-C₄cycloalkyl. In some embodiments, a cycloalkyl is a C₁-C₆ cycloalkyl. Non-limiting examples of fused bicyclic cycloalkyl include: bicyclo[4.4.0]decane, bicyclo[4.3.0]nonane, bicyclo[4.2.0]octane, bicyclo[4.1.0]heptane, bicyclo[3.3.0]octane, or bicyclo[3.1.0]hexane. Non-limiting examples of spirocyclic cycloalkyls include: spiro[2.2]pentyl, spiro[3.2]hexyl, spiro[3.3]heptyl, spiro[4.3]octyl, spiro[4.2]heptyl, spiro[5.3]nonyl, spiro[5.4]decanyl, spiro[5.5]undecanyl, spiro[5.2]octyl, spiro[6.3]decanyl, spiro[6.4]undecanyl, or spiro[6.5]dodecanyl. Non-limiting examples of bridged bicyclic cycloalkyl include: bicyclo[1.1.1]pentanyl, bicyclo[2.1.1]hexanyl, bicyclo[3.1.1]heptanyl, bicyclo[2.2.1]heptanyl, bicyclo[2.2.2]octanyl, bicyclo[3.2.2]octanyl, bicyclo[3.3.1]nonanyl, bicyclo[4.2.2]dodecanyl, bicyclo[3.3.2]dodecanyl, bicyclo[3.2.2]nonanyl, bicyclo[3.3.3]undecanyl, or bicyclo[3.1.1]heptanyl.

The term “cycloalkenyl” refers to a type of non-aromatic cycloalkyl group in which at least one carbon-carbon double bond is present. In some embodiments, cycloalkenyl is a monocyclic cycloalkenyl or bicyclic cycloalkenyl. In some embodiments, cycloalkenyl is a monocyclic cycloalkenyl. In some embodiments, cycloalkenyl is a bicyclic cycloalkenyl. In some embodiments, a bicyclic cycloalkenyl is a fused bicyclic cycloalkenyl, spirocyclic cycloalkenyl, or a bridged bicyclic cycloalkenyl.

Non-limiting examples of monocyclic cycloalkenyls include: cyclopentenyl, cyclohexenyl, cycloheptenyl, or cyclooctenyl. In some embodiments, monocyclic cycloalkenyls include:

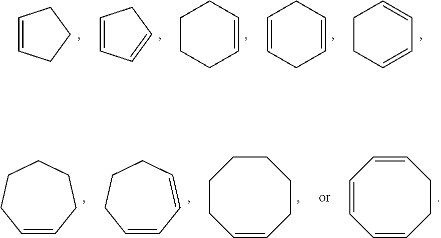

Non-limiting examples of spirocyclic cycloalkenyls include:

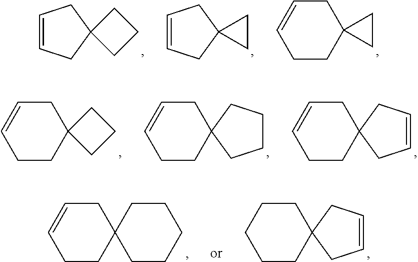

Non-limiting examples of fused bicyclic cycloalkenyl include:

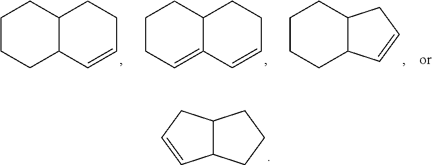

Non-limiting examples of bridged bicyclic cycloalkenyl include:

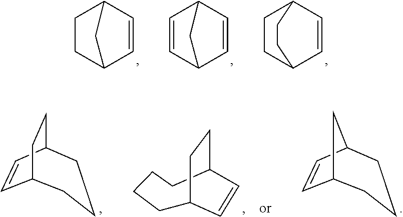

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a —C₁-C₆ fluoroalkyl.

The term “heterocycle” or“heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 12 atoms in its ring system, and with the proviso that any ring does not contain two adjacent O or S atoms. Non-aromatic heterocyclic groups (also known as heterocycloalkyls) can be fully saturated (i.e., no units of unsaturation, also known as heterocycloalkyl), partially unsaturated (i.e., at least one unit of unsaturation), or fully unsaturated (i.e., heteroaryl). In some embodiments, fully saturated or partially unsaturated heterocyclic groups include rings having 3 to 12 atoms in its ring system and aromatic heterocyclic groups include rings having 5 to 10 atoms in its ring system. In some embodiments, at least one of the two rings of a partially unsaturated bicyclic heterocycle is aromatic. The heterocyclic groups include partially unsaturated bicyclic heterocyclic rings wherein the heterocycloalkyl is fused with an aryl or heteroaryl. In some embodiments, one of the two rings of a bicyclic partially unsaturated heterocycle is aromatic, and the point of attachment to the remaining of the compound is at a carbon atom or heteroatom of the aromatic ring. In some embodiments, one of the two rings of a bicyclic heterocycle is aromatic, and the point of attachment to the remaining of the compound is at a carbon atom or heteroatom of the non-aromatic ring. In some embodiments, bicyclic heterocycle is a fused bicyclic heterocycle, bridged bicyclic heterocycle, or a spirocyclic bicyclic heterocycle. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl

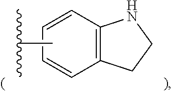

isoindolinyl

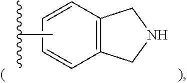

2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). Examples of partially unsaturated bicyclic heterocyclic rings are indolinyl

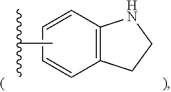

isoindolinyl

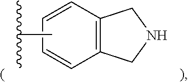

1,2-dihydroisoquinolinyl

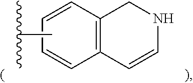

1,2,3,4-tetrahydroquinolinyl

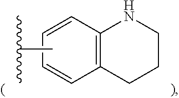

1,2,3,4-tetrahydroisoquinolinyl

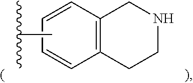

1,2,3,4-tetrahydroquinoxalinyl

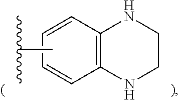

1,2,3,4-tetrahydro-1,8-naphthyridine

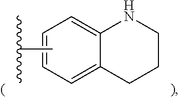

5,6,7,8-tetrahydro-1,7-naphthyridinyl

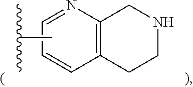

5,6,7,8-tetrahydro-1,6-naphthyridinyl

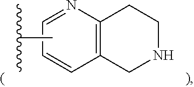

5,6,7,8-tetrahydro-1,5-naphthyridinyl

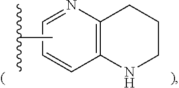

1,2,3,4-tetrahydro-1,6-naphthyridinyl

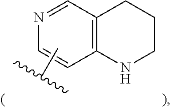

1,2,3,4-tetrahydro-2,6-naphthyridinyl

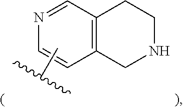

1,2,3,4-tetrahydro-3,6-naphthyridinyl

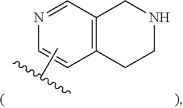

1,2,3,4-tetrahydro-4,6-naphthyridinyl

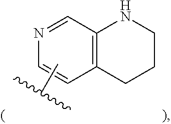

or 2,3-dihydrobenzo[d]oxazole

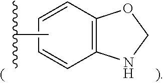

Partially unsaturated bicyclic heterocyclic groups are optionally substituted with one or two oxo (═) moieties, such as pyrrolidin-2-one, indolin-2-one

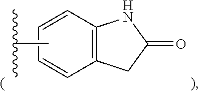

isoindolin-1-one

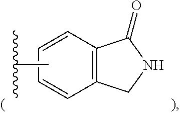

benzo[d]oxazol-2(3H)-one

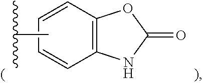

3,4-dihydroquinolin-2(1H)-one

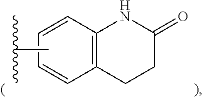

or 3,4-dihydroisoquinolin-1(2H)-one

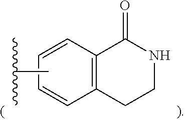

The terms “heteroaryl” or, alternatively, “heteroaromatic” refer to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizinyl, indolyl, benzofuranyl, benzothiophenyl, indazolyl, benzimidazolyl, purinyl, quinolizinyl, quinolinyl, isoquinolinyl, cinnolinyl

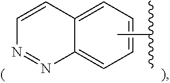

phthalazinyl

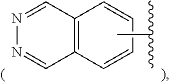

quinazolinyl

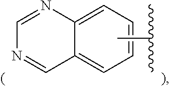

quinoxalinyl

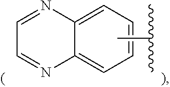

1,8-naphthyridinyl

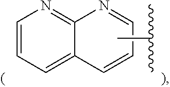

1,7-naphthyridinyl

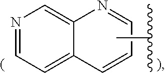

1,6-naphthyridinyl

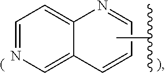

1,5-naphthyridinyl

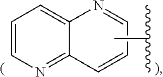

pteridinyl

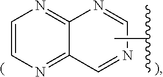

and tetrazolo[1,5-a]pyridinyl

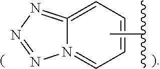

In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, a heteroaryl contains 1 O atom. In some embodiments, a heteroaryl contains 1 S atom in the ring. In some embodiments, a heteroaryl is a 5 to 10-membered heteroaryl. In some embodiments, a monocyclic heteroaryl is a 5 to 6 membered heteroaryl. In some embodiments, a monocyclic heteroaryl is a 5-membered heteroaryl. In some embodiments, a monocyclic heteroaryl is a 6-membered heteroaryl. In some embodiments, a bicyclic heteroaryl is a 10-membered heteroaryl.

A “heterocycloalkyl” group refers to a cycloalkyl group that includes at least one heteroatom selected from nitrogen, oxygen and sulfur. A “heterocycloalkyl” group is optionally substituted with one or two oxo (═O) moieties, such as pyrrolidin-2-one. In some embodiments, heterocycloalkyl is a monocyclic heterocycloalkyl or bicyclic heterocycloalkyl. In some embodiments, heterocycloalkyl is a monocyclic heterocycloalkyl. In some embodiments, heterocycloalkyl is a bicyclic heterocycloalkyl. In some embodiments, the bicyclic cycloalkyl is a spiro bicyclic (i.e., spirocyclic) heterocycloalkyl, a fused bicyclic heterocycloalkyl, or a bridged bicyclic heterocycloalkyl. In one aspect, a heterocycloalkyl is a 3 to 12-membered heterocycloalkyl. In another aspect, a heterocycloalkyl is a 5 to 10-membered heterocycloalkyl. In some embodiments, a heterocycloalkyl is a 5-membered heterocycloalkyl. In some embodiments, a heterocycloalkyl is a 6-membered heterocycloalkyl. In some embodiments, a heterocycloalkyl is monocyclic or bicyclic. In some embodiments, a heterocycloalkyl is a 10-membered bicyclic heterocycloalkyl. In some embodiments, a heterocycloalkyl is a 9-membered bicyclic heterocycloalkyl. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-4 nitrogen (N) atoms in the ring. In some embodiments, a heterocycloalkyl contains 0-2 N atoms, 0-2 oxygen (O) atoms and 0-1 sulfur (S) atoms in the ring.

Monocyclic heterocycloalkyls that include 2 to 10 carbon atoms in the ring include aziridinyl, azetidinyl, pyrrolidinyl, piperidinyl, azepanyl, oxetanyl, thietanyl, thioxanyl, oxazolidinonyl, dihydrofuranyl, tetrahydrofuranyl, tetrahydrothienyl, dihydropyranyl, tetrahydropyranyl, 2H-pyranyl, 4H-pyranyl, tetrahydrothiopyranyl, morpholinyl, thiomorpholinyl, piperazinyl, homopiperidinyl, oxepanyl, thiepanyl, oxazepinyl, diazepinyl, thiazepinyl, 1,2,3,6-tetrahydropyridinyl, piperidin-2-onyl, pyrrolidine-2,5-dithionyl, pyrrolidine-2,5-dionyl, pyrrolidinonyl, imidazolidinyl, imidazolidin-²-onyl, thiazolidin-²-onyl, pyrrolin-2-yl, pyrrolin-3-yl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, or imidazolidinyl. In some embodiments, monocyclic heterocycloalkyls that include 2 to 8 carbon atoms in the ring include aziridinyl, azetidinyl, pyrrolidinyl, piperidinyl, azepanyl, oxetanyl, tetrahydrofuranyl, tetrahydrothienyl, tetrahydropyranyl, morpholinyl, thiomorpholinyl, piperazinyl, or homopiperidinyl. In one aspect, a heterocycloalkyl is a C₂-C₁₀ heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

Non-limiting examples of spirocyclic heterocycloalkyls include:

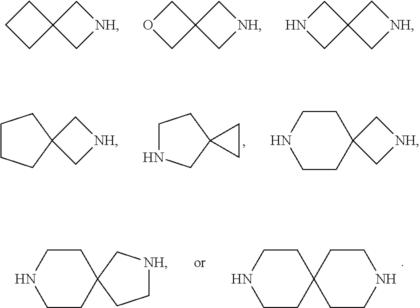

Non-limiting examples of fused bicyclic heterocycloalkyl include:

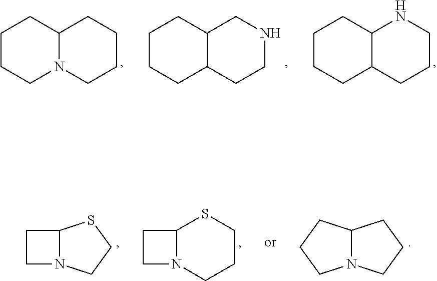

Non-limiting examples of bridged bicyclic heterocycloalkyl include:

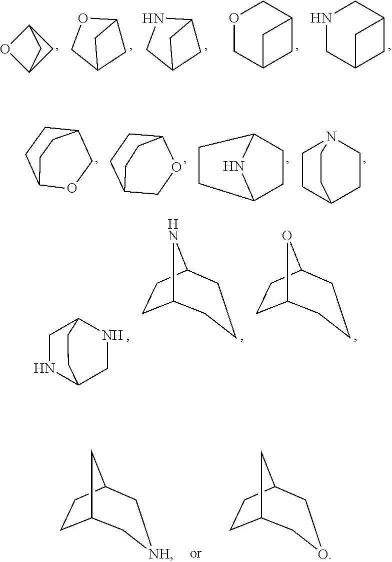

The term “bond” or “single bond” refers to a chemical bond between two atoms, or two moieties when the atoms joined by the bond are considered to be part of a larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, —C₁-C₄ alkyl, C₃-C₆ cycloalkyl, —C₁-C₄ fluoroalkyl, —C₁-C₄ heteroalkyl, —C₁-C₄ alkoxy, —C₁-C₄ fluoroalkoxy, —SC₁-C₄ alkyl, —S(═O)C₁-C₄ alkyl, and —S(═O)₂C₁-C₄ alkyl. In some embodiments, optional substituents are independently selected from halogen, —CN, -Nit, —OH, —NH(CH), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “article of manufacture” and “kit” are used as synonyms.

The term “subject” or“patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

Chemical structures depicted herein include all stereochemical forms of the structure, unless otherwise stated.

The term “peptide” as used herein refers to a compound comprising two or more amino acids in a serial array, linked through peptide bonds. The amino acids making up the polypeptide may be naturally derived, or may be synthetic.

The term “amino acid” as used herein refers to both natural and unnatural amino acids. The term “unnatural amino acid” as used herein refers to an amino acid that is not part of the 20 amino acids that occur naturally in protein.

As used herein, amino acid residue refers to an amino acid formed upon chemical digestion (hydrolysis) of a polypeptide at its peptide linkages. The amino acid residues described herein are, in certain embodiments, in the “L” isomeric form. Residues in the “D” isomeric: form can be substituted for any “L” amino acid residue, as long as the desired functional property is retained by the polypeptide. “—NH₂” refers to the free amino group present at the amino terminus of a polypeptide. “—CO₂H” refers to the free carboxy group present at the carboxyl terminus of a polypeptide. In keeping with standard polypeptide nomenclature described in J. Biol. Chem., 243:3552 59 (1969) and adopted at 37 C.F.R. §§ 1.821-1.822, abbreviations for amino acid residues are shown in the following Table A:

TABLE A
Amino Acid Abbreviations

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gin |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino terminus to carboxyl terminus. In addition, the phrase “amino acid residue” is broadly defined to include the amino acids listed in the Table of Correspondence and modified and unusual amino acids, such as those referred to in 37 C.F.R. §§ 1.821-1.822, and incorporated herein by reference. Furthermore, it should be noted that a dash at the beginning or end of an amino acid residue sequence indicates a. peptide bond to a further sequence of one or more amino acid residues or to an amino terminal group Such as —NH₂ or to a carboxyl terminal group such as —CO₂H.

In a peptide, suitable conservative substitutions of amino acids are known to those of skill in this art and can be made generally without altering the biological activity of the resulting Molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in Table B as follows:

TABLE B

| Original residue | Conservative substitution |
|---|---|
| Ala (A) | Gly; Ser |
| Arg (R) | Lys |
| Asn (N) | Gln; His |
| Asp (D) | Glu |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

TABLE C
Representative amino acid side chains

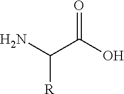

R
—H
Glycine (Gly)

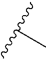

Alanine (Ala)

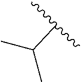 [SMILES: *C(C)C]

Valine (Val)

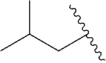 [SMILES: *CC(C)C]

Leucine (Leu)

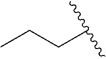 [SMILES: *CCC]

Norvaline (Nva)

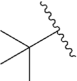 [SMILES: *C(C)(C)C]

tert-Leucine (Tle)

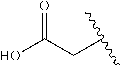 [SMILES: *CC(=O)O]

Aspartic Acid (Asp)

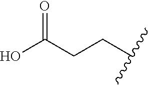 [SMILES: *CCC(=O)O]

Glutamic acid (Glu)

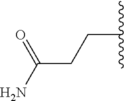 [SMILES: *CCC(N)=O]

Glutamine (Gln)

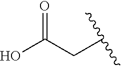 [SMILES: *CC(=O)O]

Asparagine (Asn)

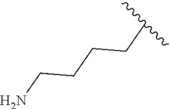 [SMILES: *CCCCN]

Lysine (Lys)

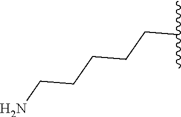 [SMILES: *CCCCCN]

Homolysine (HLys)

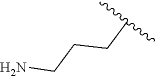 [SMILES: *CCCN]

Ornithine (Orn)

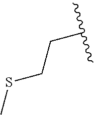 [SMILES: *CCSC]

Methionine (Met)

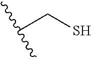 [SMILES: *CS]

Cysteine (Cys)

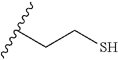 [SMILES: *CCS]

Homocysteine (HCys)

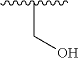 [SMILES: *CO]

Serine (Ser)

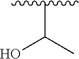 [SMILES: *C(C)O]

Threonine (Thr)

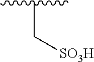 [SMILES: *CS(=O)(=O)O]

Cysteic acid

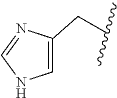 [SMILES: *Cc1c[nH]cn1]

Histidine (His)

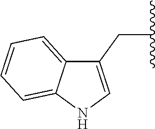 [SMILES: *Cc1c[nH]c2ccccc12]

Tryptophan (Trp)

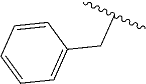 [SMILES: *Cc1ccccc1]

Phenylalanine (Phe)

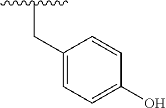 [SMILES: *Cc1ccc(O)cc1]

Tyrosine (Tyr)

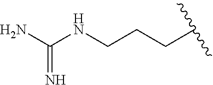 [SMILES: *CCCNC(=N)N]

Arginine (Arg)

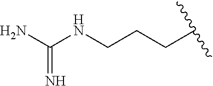 [SMILES: *CCCNC(=N)N]

Homoarginine (HArg)

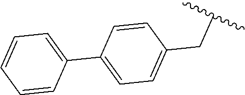 [SMILES: *Cc1ccc(-c2ccccc2)cc1]

Biphenylalanine (Bip)

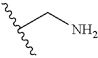 [SMILES: *CN]

3-amino alanine (Dap)

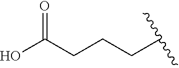 [SMILES: *CCCC(=O)O]

homoglutamic acid (Aad)

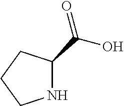

aminopimelic acid (Apm)

Additional amino acids are shown in Table D.

TABLE D
Representative cyclic and unnatural amino acids

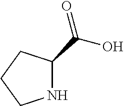

Proline (Pro)

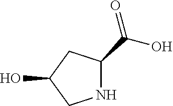

Hydroxyproline (Hyp)

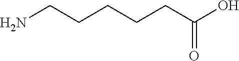

6-aminohexanoic acid (Ahx)

### NUMBERED EMBODIMENTS

Embodiment 1. A compound of Formula (A), or a pharmaceutically acceptable salt thereof:

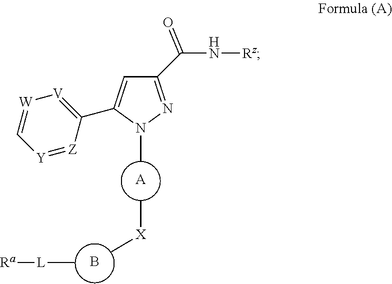

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R² substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R² substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)—, ═S(═NH)(═NH), —CH═CH—C(═O)—, or —CH₂CH₂—C(═O)—;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
W is —C(R^{r})— or —N— and Y is —C(R^{s})— or —N—; wherein R^{r} and R^{s} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—, wherein R^{x} and R^{y} are each independently selected from the group consisting of: —F, —Cl, —Br, —CF₃, —C₁-C₆ alkyl, —O—C₁-C₆ alkyl, —C₃-C₆ cycloalkyl, aryl and heteroaryl.
R^{z} is

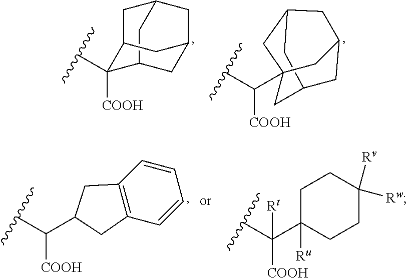

R^{v} and R^{w} are each independently —F, —Cl, or —CH₃ and R^{t} and R^{u} are each independently —C₁-C₄ alkyl; or
R^{v} is —F, —Cl, or —CH₃; R¹ is —C₁-C₄ alkyl; and R^{u} and R^{w} come together with the carbon atoms to which they are attached to form a cyclopentyl or cyclohexyl ring
L is a linker; and
R^{a} is a chelating moiety or a radionuclide complex thereof.

Embodiment 2. The compound of embodiment 1, or a pharmaceutically acceptable salt thereof, wherein W is —N— and Y is —CH—.

Embodiment 3. The compound of embodiment 1, or a pharmaceutically acceptable salt thereof, wherein W and Y are —CH—.

Embodiment 4. The compound of any one of embodiments 1-3, or a pharmaceutically acceptable salt thereof, wherein: V is —C(R^{x})— and Z is —C(R^{y})—.

Embodiment 5. The compound of any one of embodiments 1-4, or a pharmaceutically acceptable salt thereof, wherein: R^{x} and R^{y} are each independently selected from the group consisting of: —F, —Cl, —Br, —CF₃, —C₁-C₆ alkyl, and —O—C₁-C₆ alkyl.

Embodiment 6. The compound of any one of embodiments 1-4, or a pharmaceutically acceptable salt thereof, wherein: R^{x} is —F, —Cl, —CF₃, —CH₃, —CH₂CH₃, —OCH₃, cyclopropyl, or 5 to 6-membered heteroaryl.

Embodiment 7. The compound of any one of embodiments 1-4, or a pharmaceutically acceptable salt thereof, wherein R^{x} is pyrimidinyl.

Embodiment 8. The compound of any one of embodiments 1-4 or 6-7, or a pharmaceutically acceptable salt thereof, wherein: R¹ is —OCH³.

Embodiment 8. The compound of any one of embodiments 1-4, or a pharmaceutically acceptable salt thereof, wherein: R^{x} is —F, —Cl, —CF₃, —CH₃, —CH₂CH₃, —OCH₃, cyclopropyl, or pyrimidinyl and R^{y} is —OCH₃.

Embodiment 10. The compound of any one of embodiments 1-4, or a pharmaceutically acceptable salt thereof, wherein: R^{x} is —F, —Cl, or —OCH₃, and R^{y} is —OCH₃.

Embodiment 11. The compound of embodiment 1, or a pharmaceutically acceptable salt thereof, wherein: W is —CH— or —N—; Y is —CH—; R^{x} is —OCH₃, and R^{y} is —OCH₃.

Embodiment 12. The compound of embodiment 1, or a pharmaceutically acceptable salt thereof, wherein:
V is —C(R^{x})—;
Z is —C(R^{y})—;
R^{x} is selected from the group consisting of: —F, —Cl, —CF₃, —C₁-C₆ alkyl, —O—C₁-C₆ alkyl, —C₃-C₆ cycloalkyl, and heteroaryl; and
R^{y} is —O—C₁-C₆ alkyl.

Embodiment 13. The compound of any one of embodiments 1-3, or a pharmaceutically acceptable salt thereof, wherein: V is —C(R^{x})—; Z is —C(R^{y})—; and R^{x} and R^{y} are each independently —O—C₁-C₆ alkyl.

Embodiment 14. The compound of any one of embodiments 1-13, or a pharmaceutically acceptable salt thereof, wherein R^{z} is

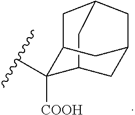 [SMILES: *C1(C(=O)O)C2CC3CC(C2)CC1C3]

Embodiment 15. The compound of any one of embodiments 1-13, or a pharmaceutically acceptable salt thereof, wherein R^{z} is

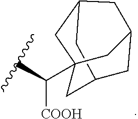 [SMILES: *[C@H](C(=O)O)C12CC3CC(CC(C3)C1)C2]

Embodiment 16. The compound of any one of embodiments 1-13, or a pharmaceutically acceptable salt thereof, wherein R^{z} is

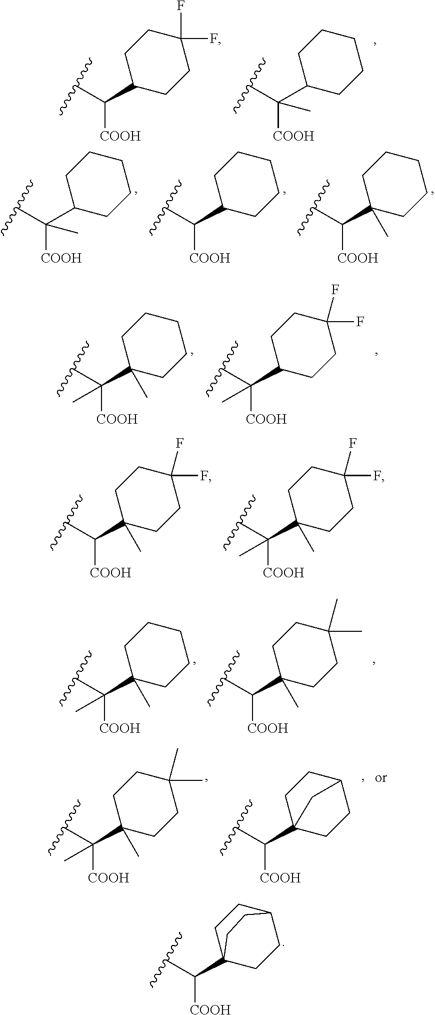

Embodiment 17. The compound of embodiment 1, wherein the compound of Formula (A) is a compound of Formula (I), or a pharmaceutically acceptable salt thereof:

wherein:

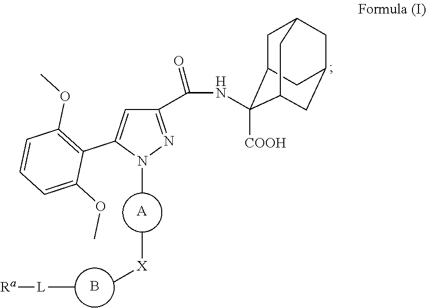

Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O).
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, or 3,6-diazabicyclo[3.1.1]heptanyl, wherein Ring B is optionally substituted with one or more R² substituents; and wherein any saturated carbon of Ring B is optionally substituted with oxo (═O).
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —C(═O)NR³R⁴, —CH(═N)OU, —NR⁵R⁶, —NO₂, —NR⁷C(═O)R^{x}, —NR⁹C(═O)OR¹⁰, and —OC(═O)NR¹¹R¹²;
X is absent, —O—, —NH—, —S—, —S(═O)(═NH)—, ═S(═NH)(═NH), —CH═CH—C(═O)—, or —CH₂CH₂—C(═O)—;
each R² is independently selected from the group consisting of —F, substituted or unsubstituted C₁-C₆ alkyl, —CF₃, or —CN, or two R^{t} variables on the same or adjacent carbon atoms come together to form an unsubstituted C₃-C₆ cycloalkyl ring;
R³, R⁴, R⁵, R⁶, R⁷, R⁸, R⁹, R¹⁰, R¹¹ and R¹² are each independently —H or —CH₃;
L is a linker; and
R^{a} is a chelating moiety or a radionuclide complex thereof.

Embodiment 18. The compound of any one of embodiments 1-17, or a pharmaceutically acceptable salt thereof, wherein Ring A is phenyl, naphthyl, or a bicyclic heteroaryl ring; wherein each Ring A is optionally substituted with 1-6 R¹ substituents.

Embodiment 19. The compound of any one of embodiments 1-17, or a pharmaceutically acceptable salt thereof, wherein Ring A is phenyl, naphthyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, or 1,5-naphthyridinyl; wherein each Ring A is optionally substituted with 1-6 R¹ substituents.

Embodiment 20. The compound of any one of embodiments 1-17, or a pharmaceutically acceptable salt thereof, wherein Ring A is a partially unsaturated bicyclic heterocyclic ring; wherein any saturated carbon of Ring A is optionally substituted with oxo (═O) and wherein each Ring A is optionally substituted with 1-6 R¹ substituents.

Embodiment 21. The compound of any one of embodiments 1-17, or a pharmaceutically acceptable salt thereof, wherein Ring B is absent and Ring A is a partially unsaturated bicyclic heterocyclic ring selected from indolinyl, isoindolinyl, 1,2-dihydroisoquinolinyl, 1,2,3,4-tetrahydroquinolinyl, 1,2,3,4-tetrahydroisoquinolinyl, 1,2,3,4-tetrahydroquinoxalinyl, 1,2,3,4-tetrahydro-1,8-naphthyridine, 5,6,7,8-tetrahydro-1,7-naphthyridinyl, 5,6,7,8-tetrahydro-1,6-naphthyridinyl, 5,6,7,8-tetrahydro-1,5-naphthyridinyl, 1,2,3,4-tetrahydro-1,6-naphthyridinyl, 1,2,3,4-tetrahydro-2,6-naphthyridinyl, 1,2,3,4-tetrahydro-3,6-naphthyridinyl, or 1,2,3,4-tetrahydro-4,6-naphthyridinyl, or 2,3-dihydrobenzo[d]oxazolyl; wherein any saturated carbon of Ring A is optionally substituted with oxo (═O) and wherein each Ring A is optionally substituted with 1-6 R¹ substituents.

Embodiment 22. The compound of any one of embodiments 1-17, or a pharmaceutically acceptable salt thereof, wherein Ring A is phenyl, wherein Ring A is optionally substituted with 1-6 R¹ substituents.

Embodiment 23. The compound of any one of embodiments 1-17, or a pharmaceutically acceptable salt thereof, wherein Ring A is naphthyl, wherein Ring A is optionally substituted with 1-6 R¹ substituents.

Embodiment 24. The compound of any one of embodiments 1-17, or a pharmaceutically acceptable salt thereof, wherein Ring A is tetrahydroisoquinolinyl, wherein Ring A is optionally substituted with 1-6 R¹ substituents.

Embodiment 25. The compound of any one of embodiments 1-24, or a pharmaceutically acceptable salt thereof, wherein each R¹ is independently selected from the group consisting of —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

Embodiment 26. The compound of any one of embodiments 1-24, or a pharmaceutically acceptable salt thereof, wherein each R¹ is independently selected from the group consisting of —F, —Cl, —Br, —I, —OH and —C₁-C₆ alkyl.

Embodiment 27. The compound of any one of embodiments 1-24, or a pharmaceutically acceptable salt thereof, wherein R¹ is —CH(CH₃)₂.

Embodiment 28. The compound of any one of embodiments 1-27, or a pharmaceutically acceptable salt thereof, wherein X is a bond.

Embodiment 29. The compound of any one of embodiments 1-27, or a pharmaceutically acceptable salt thereof, wherein X is —O— or —NH—.

Embodiment 30. The compound of any one of embodiments 1-27, or a pharmaceutically acceptable salt thereof, wherein X is —S(═O)(═NH)— or ═S(═NH)(═NH)—.

Embodiment 31. The compound of any one of embodiments 1-30, or a pharmaceutically acceptable salt thereof, wherein Ring B is piperidinyl, pyrrolidinyl, or azetidinyl, wherein Ring B is optionally substituted with one or more R² substituents; and wherein any saturated carbon of Ring B is optionally substituted with oxo (═O).

Embodiment 32. The compound of any one of embodiments 1-30, or a pharmaceutically acceptable salt thereof, wherein Ring B is substituted or unsubstituted piperazinyl, wherein Ring B is optionally substituted with one or more R² substituents.

Embodiment 33. The compound of any one of embodiments 1-30, or a pharmaceutically acceptable salt thereof, wherein Ring B is unsubstituted piperidinyl.

Embodiment 34. The compound of any one of embodiments 1-33, or a pharmaceutically acceptable salt thereof, wherein each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring.

Embodiment 35. The compound of any one of embodiments 1-33, or a pharmaceutically acceptable salt thereof, wherein each R² is independently —F or substituted or unsubstituted —C₁-C₆ alkyl.

Embodiment 36. The compound of any one of embodiments 1-33, or a pharmaceutically acceptable salt thereof, wherein R² is —CH₂NH₂.

Embodiment 37. The compound of any one of embodiments 1-27, or a pharmaceutically acceptable salt thereof, wherein —X-Ring B— is a bond,

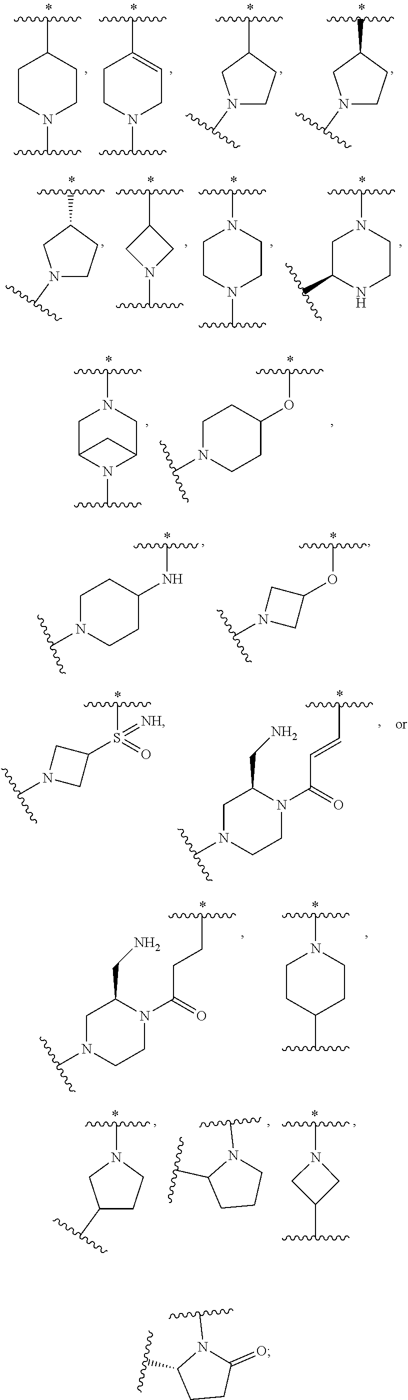

wherein * denotes the attachment point of —X-Ring B— to Ring A.

Embodiment 38. The compound of embodiment 1 or 17, or a pharmaceutically acceptable salt thereof, wherein the compound has the structure of Formula (IIa), Formula (IIIa), or Formula (IVa):

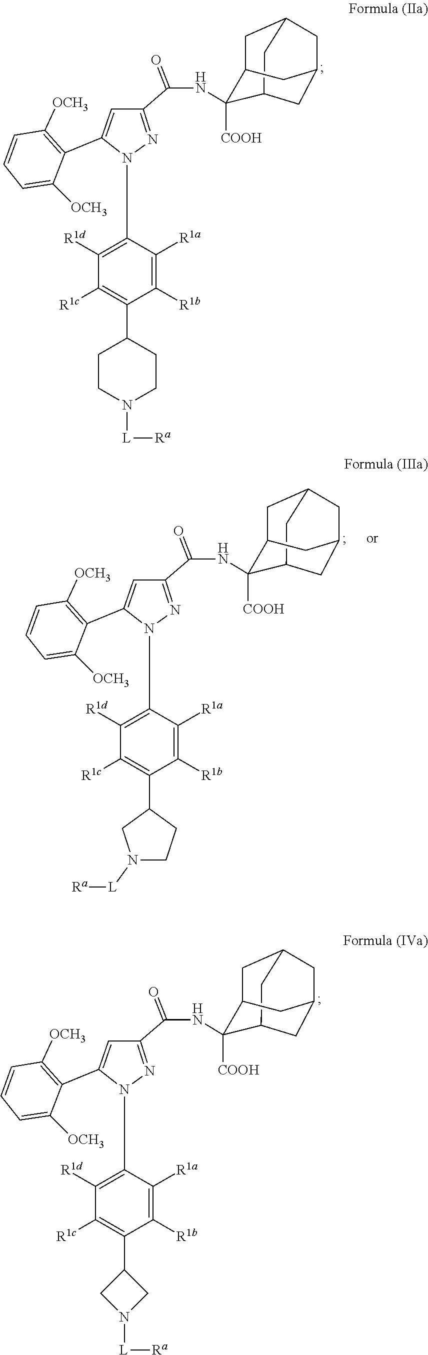

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═)OCH₃, —C(═O)NH₂, —CH(═N)OH, —NH₂, —NHCH₃, —N(CH₃)₂, —NO₂, —NHC(═O)CH₃. —NHCO₂H, —NHC(═O)OCH₃, —OC(═O)NH₂, and substituted or unsubstituted —C₁-C₆ alkyl.

Embodiment 39. The compound of embodiment 1 or 17, or a pharmaceutically acceptable salt thereof, wherein the compound has the structure of Formula (Jib), Formula (IIIb), or Formula (IVb):

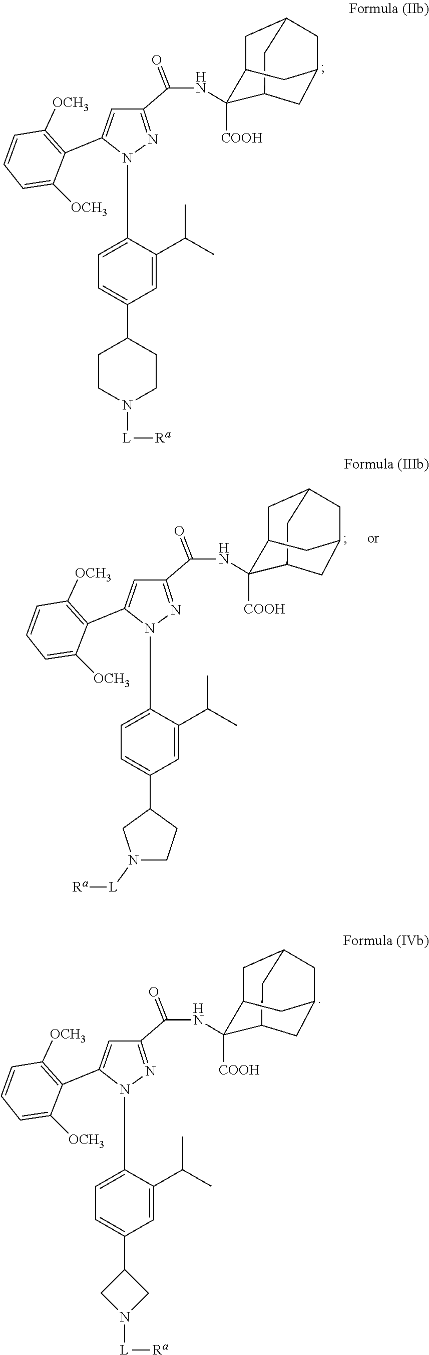

Embodiment 40. The compound of embodiment 1 or 17, or a pharmaceutically acceptable salt thereof, wherein the compound has the structure of Formula (IIIc) or Formula (IIe):

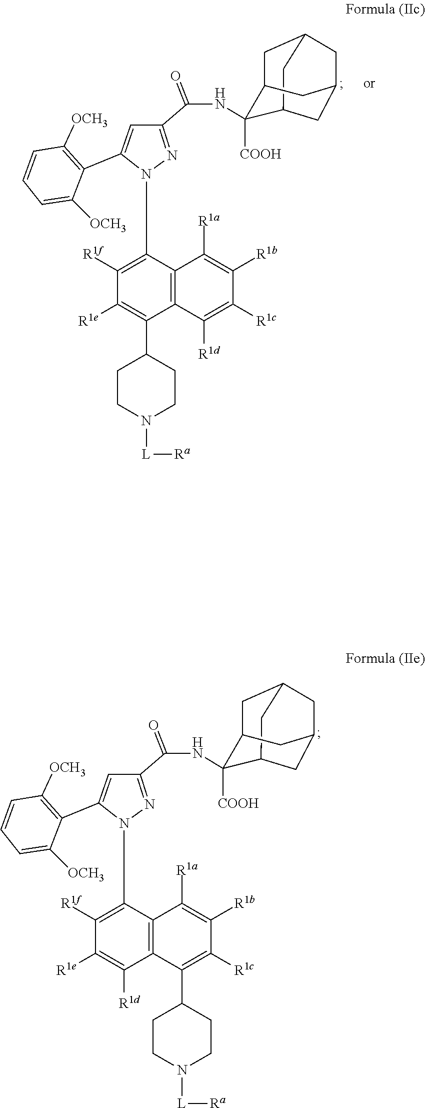

wherein R^{1a}, R^{1b}, R^{c}, R^{1d}, R^{1e}, and R^{1f} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

Embodiment 41. The compound of embodiment 1 or 17, or a pharmaceutically acceptable salt thereof, wherein the compound has the structure of Formula (IId), Formula (IIf), Formula (IIId), or Formula (IVd):

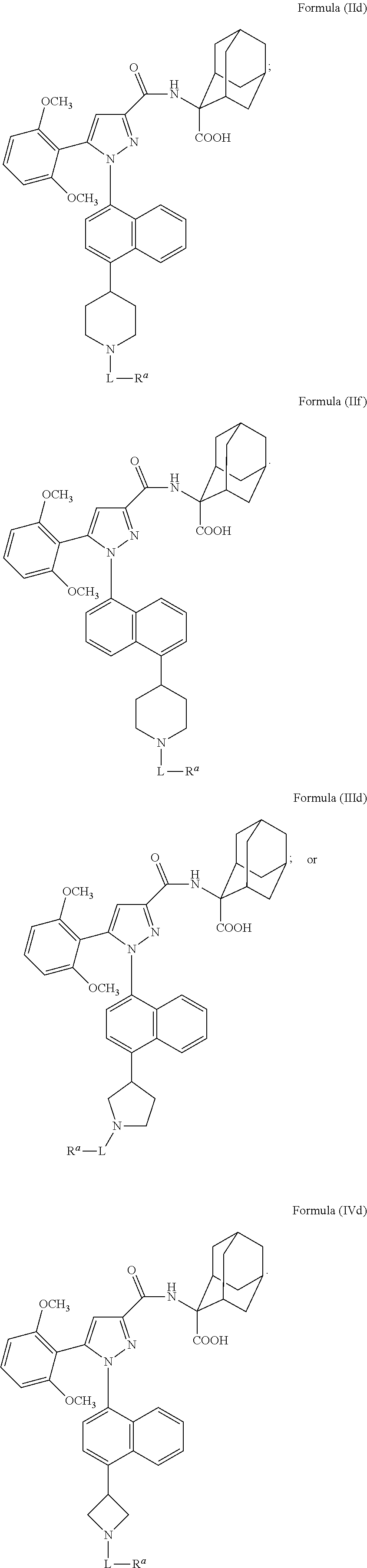

Embodiment 42. The compound of embodiment 1 or 17, or a pharmaceutically acceptable salt thereof, wherein the compound has the structure of Formula (IIg):

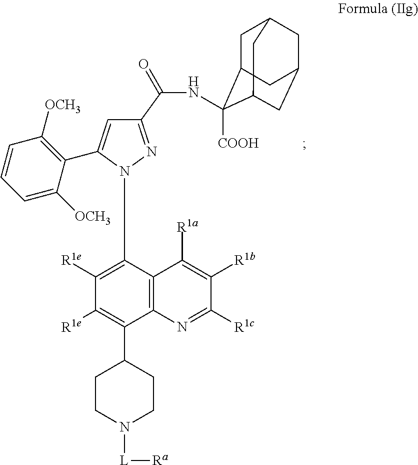

wherein R^{1a}, R^{1b}, R^{1c}, R^{1d}, and R^{1e} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

Embodiment 43. The compound of embodiment 1 or 17, or a pharmaceutically acceptable salt thereof, wherein the compound has the structure of Formula (IIh):

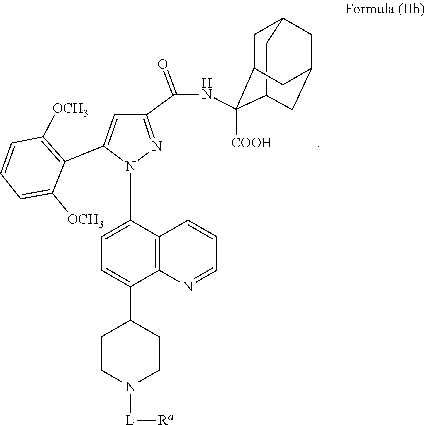

Embodiment 44. The compound of embodiment 1 or 17, or a pharmaceutically acceptable salt thereof, wherein the compound has the structure of Formula (Vb), Formula (Vd), Formula (Vf), Formula (IXb), Formula (Xb), or Formula (XIb):

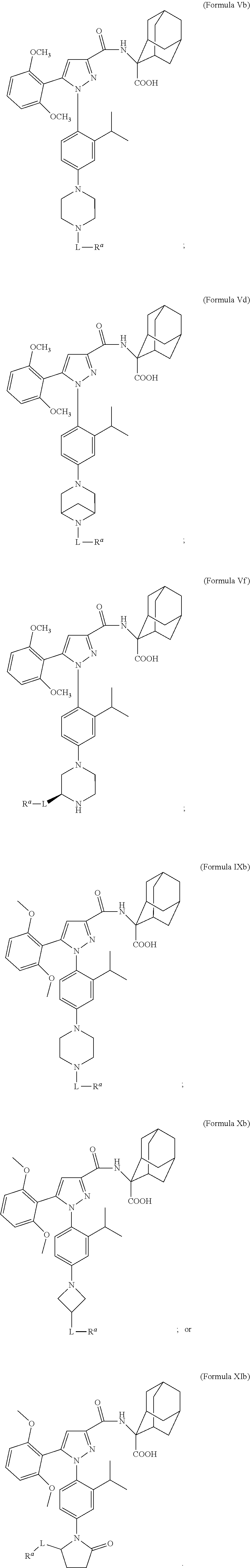

Embodiment 45. The compound of embodiment 1 or 17, or a pharmaceutically acceptable salt thereof, wherein the compound has the structure of Formula (VIb); Formula (VId); or Formula (VIf):

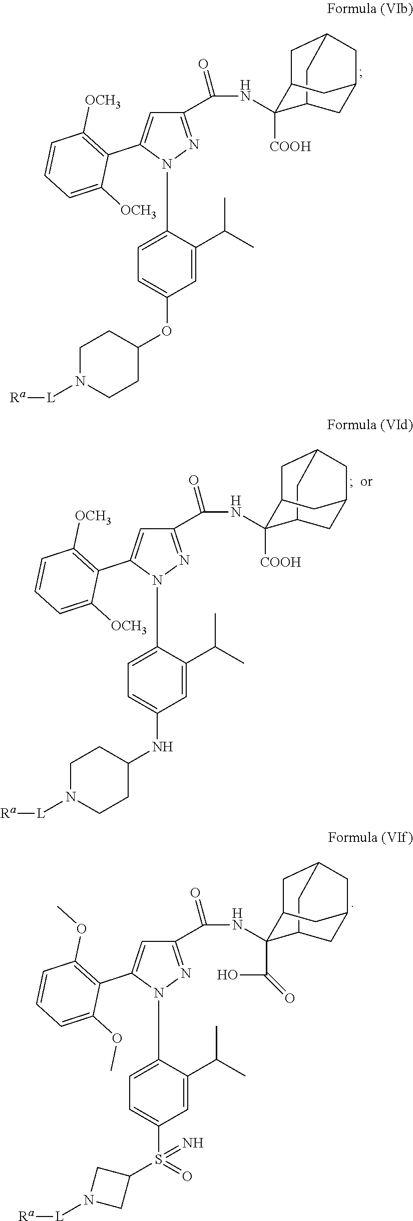

Embodiment 46. The compound of embodiment 1 or 17, or a pharmaceutically acceptable salt thereof, wherein the compound has the structure of Formula (VIIa):

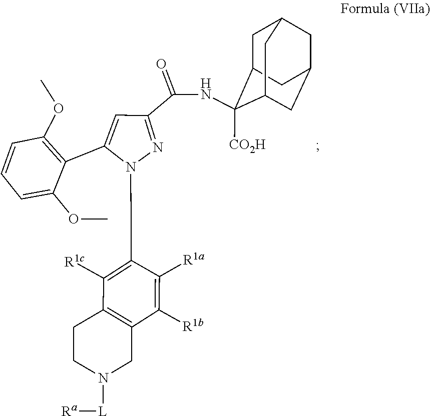

Embodiment 47. The compound of embodiment 1 or 17, or a pharmaceutically acceptable salt thereof, wherein the compound has the structure of Formula (VIIb):

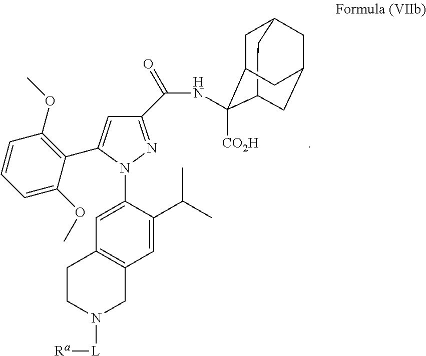

Embodiment 48. The compound of embodiment 1, or a pharmaceutically acceptable salt thereof, wherein the compound has the structure of Formula (XIIb) or Formula (XVIb):

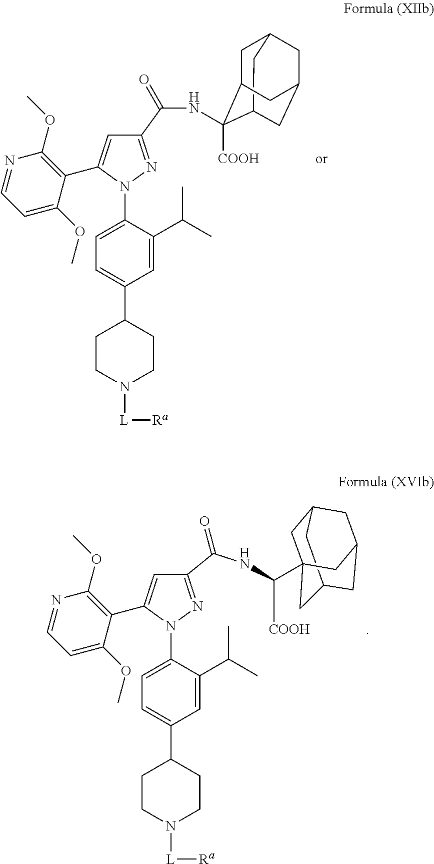

Embodiment 49. The compound of embodiment 1, or a pharmaceutically acceptable salt thereof, wherein the compound has the structure of Formula (XIIIb) or Formula (XIVb):

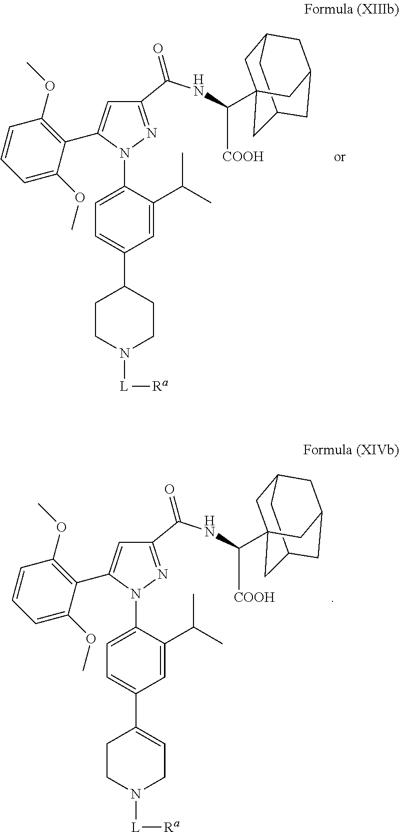

Embodiment 50. The compound of embodiment 1, or a pharmaceutically acceptable salt thereof, wherein the compound has the structure of Formula (XIId):

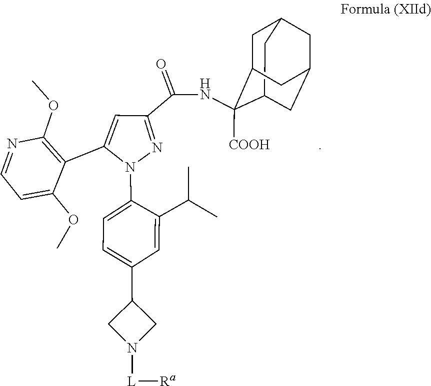

Embodiment 51. The compound of embodiment 1, or a pharmaceutically acceptable salt thereof, wherein the compound has the following structure:

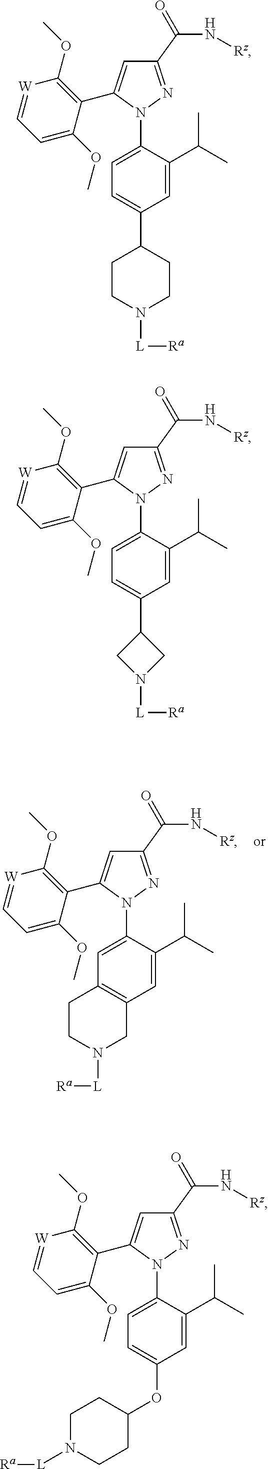

Embodiment 52. A compound of the following chemical formula, or a pharmaceutically acceptable salt thereof:

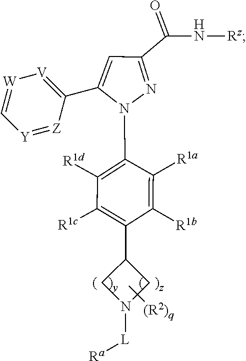

wherein:
R^{1a}, R^{1b}, R^{1c}, and R^{1d} are each independently selected from the group consisting of —F, —Cl, —Br, —I, —OH, substituted or unsubstituted alkyl, substituted or unsubstituted heteroalkyl, —CF₃, —CN, —CO₂, —CO₂CH₃, —C(═O)NR³R⁴, —CH(═O)OH, —NR⁵R⁶, —NO₂, —NR⁷C(═O)R⁸, —NR⁹C(═O)OR¹⁰, and —OC(═O)NR¹¹R¹²;
R³, R⁴, R⁵, R⁶, R⁷, R⁸, R⁹, R¹⁰, R¹ and R¹² are each independently —H or —CH₃;
   each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
y is 1 or 2;
z is 1 or 2;
q is 0, 1, 2, 3, 4, 5, 6, 7, or 8;
W is —C(R^{r})— or —N— and Y is —C(R^{s})— or —N—; wherein R^{r} and R^{s} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—, wherein R^{x} and R^{y} are each independently selected from the group consisting of: —F, —Cl, —Br, —CF₃, —C₁-C₆ alkyl, or —O—C₁-C₆ alkyl;
R^{z} is

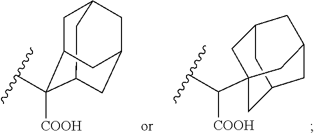 [SMILES: *C1(C(=O)O)C2CC3CC(C2)CC1C3 ; *C(C(=O)O)C12CC3CC(CC(C3)C1)C2]

L is a linker; and
R^{a} is a chelating moiety or a radionuclide complex thereof.

Embodiment 53. The compound of embodiment 52, or a pharmaceutically acceptable salt thereof, wherein W is —N— and Y is —CH— or wherein W and Y are —CH—; and V is —C(R^{x})—; Z is —C(R^{y})—; R^{x} is —F, —Cl, or —OCH₃; and R^{y} is —OCH₃.

Embodiment 54. The compound of embodiment 52, or a pharmaceutically acceptable salt thereof, wherein R^{1a}, R^{1b}, R^{1c}, and R^{1d} are each independently selected from the group consisting of —F, —Cl, —Br, —I, —OH and —C₁-C₆ alkyl.

Embodiment 55. The compound of embodiment 52, or a pharmaceutically acceptable salt thereof, wherein R^{1a} is —CH(CH₃)₂.

Embodiment 56. The compound of embodiment 52, or a pharmaceutically acceptable salt thereof, wherein q is 0.

Embodiment 57. The compound of embodiment 52, or a pharmaceutically acceptable salt thereof, wherein

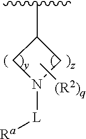

is

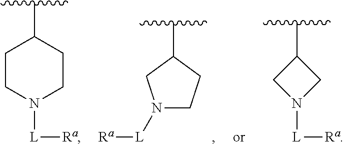

Embodiment 58. The compound of embodiment 52, or a pharmaceutically acceptable salt thereof, wherein

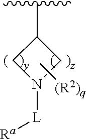

is

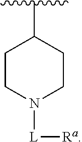

Embodiment 59. The compound of embodiment 52, or a pharmaceutically acceptable salt thereof, wherein

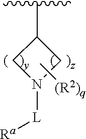

is

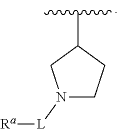

Embodiment 60. The compound of embodiment 52, or a pharmaceutically acceptable salt thereof, wherein

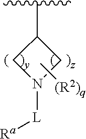

is

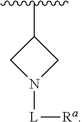

Embodiment 61a. The compound of embodiment 52, or a pharmaceutically acceptable salt thereof, wherein the compound has the structure of Formula (IIa), Formula (IIIa), Formula (IVa), Formula (XIIa), or Formula (IIc):

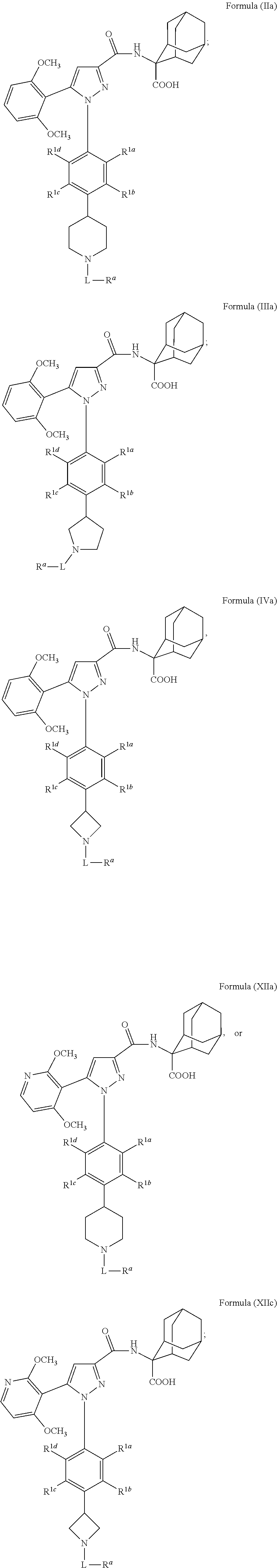

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

Embodiment 61b. The compound of embodiment 52, or a pharmaceutically acceptable salt thereof, wherein the compound has the following structure:

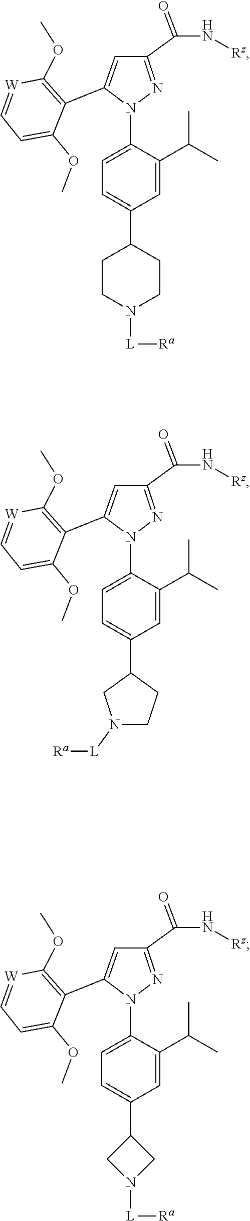

wherein W is —CH— or —N—, and R^{z} is

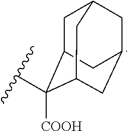 [SMILES: *C1(C(=O)O)C2CC3CC(C2)CC1C3]

Embodiment 62. The compound of any one of embodiments 1-61, or a pharmaceutically acceptable salt thereof, wherein R^{a} is a chelating moiety independently selected from the group consisting of:
1,4,7,10-tetraazacyclododecane-1,4,7,10-tetraacetic acid (DOTA);
2,2′,2″-(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DO3A);
1,4,7,10-tetraazacyclododecane-1,7-diacetic acid (DO2A);
α,α′,α″,α′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
6,6′-(((pyridine-2,6-diylbis(methylene))bis((carboxymethyl)azanediyl))bis(methylene))dipicolinic acid (H₄pypa);
H₄pypa-benzyl;
6,6′,6″,6′″-(((pyridine-2,6-diylbis(methylene))bis(azanetriyl))tetrakis(methylene))-tetrapicolinic acid (H₄py4pa);
H₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2′″-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; and
3,6,9,12-tetrakis(carboxymethyl)-3,6,9,12-tetraazatetradecanedioic acid (TTHA);
or a radionuclide complex thereof.

Embodiment 63. The compound of any one of embodiments 1-61, or a pharmaceutically acceptable salt thereof, wherein R^{a} is a chelating moiety selected from the group consisting of: 1,4,7,10-tetraazacyclododecane-1,4,7,10-tetraacetic acid (DOTA) and 1,4,7,10-tetraazacyclododecane-1,4,7-triacetic acid (DO3A); or a radionuclide complex thereof.

Embodiment 64. The compound of any one of embodiments 1-61, or a pharmaceutically acceptable salt thereof, wherein R^{a} is a chelating moiety independently selected from the group consisting of:

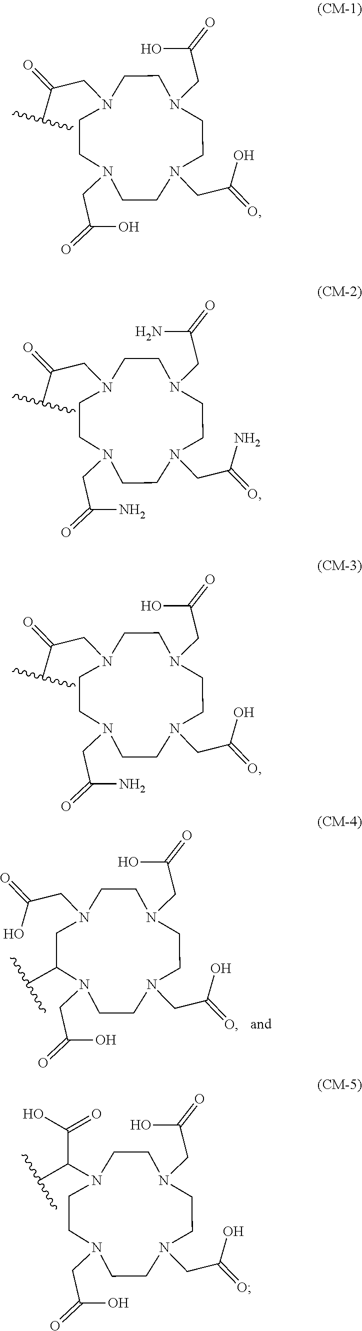 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

Embodiment 65. The compound of any one of embodiments 1-61, or a pharmaceutically acceptable salt thereof, wherein R^{a} is

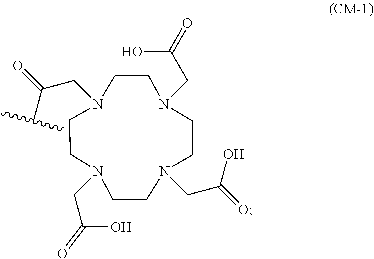 [SMILES: *C(=O)CN1CCN(CC(=O)O)CCN(CC(=O)O)CCN(CC(=O)O)CC1]

or a radionuclide complex thereof.

Embodiment 66. The compound of any one of embodiments 1-61, or a pharmaceutically acceptable salt thereof, wherein R^{a} is independently selected from:

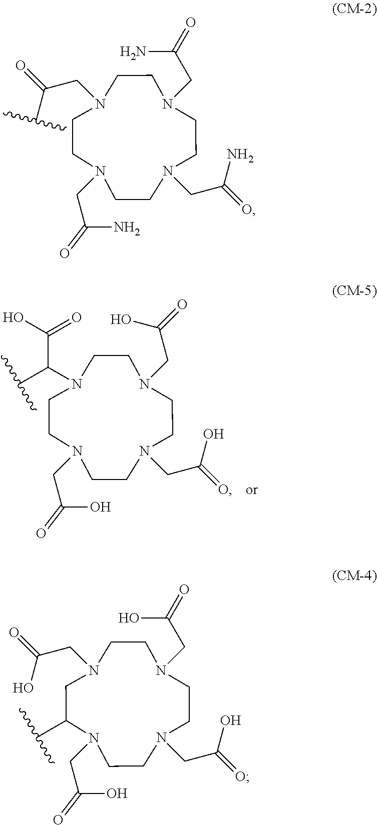 [SMILES: *C(=O)CN1CCN(CC(N)=O)CCN(CC(N)=O)CCN(CC(N)=O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]

or a radionuclide complex thereof.

Embodiment 67. The compound of any one of embodiments 1-66, wherein L is: -L², -L⁴, -L²-L⁴-, -L²-L⁶-, -L³-L⁶-, -L²-L³-L⁴-, -L²-L³-L⁶-, -L²-L⁵-L⁶-, -L²-L³-L⁵-L⁶- or -L²-L³-L⁴-L⁵-L⁶-;
L² is absent, —C₁-C₂₀ alkylene, —C₁-C₂₀ alkylene-NR¹³—, —C₁-C₂₀ alkylene-NR¹³—C(═O)—, —NR¹³—C₁-C₂₀ alkylene-, —NR¹³—C₁-C₂₀ alkylene-NR¹⁶—, —C₁-C₂₀ alkylene-O—, —O—C₁-C₂₀ alkylene-, —O—C₁-C₂₀ alkylene-NR¹⁶—, —NR¹³—C(═O)—C₁-C₂₀ alkylene-NR¹⁶—, —C₁-C₂₀ alkylene-C(═O)—, —C(═O)—C₁-C₂₀ alkylene-, —C(═O)—C₁-C₂₀ alkylene-NR¹¹—, —C(═O)—NR¹³—C₁-C₂₀ alkylene-NR¹⁶—, —C(═NH)NH—C₁-C₂₀ alkylene-NR¹³—, —C(═NH)NH—C₁-C₂₀ alkylene-, or —C(═O)—NH—O—(CH₂)ᵥ—; wherein each C₁-C₂₀ alkylene is optionally substituted with 1-3 substituents selected from the group consisting of —OH, —NH₂, and —COOH;
   each R¹³ and R¹⁶ are independently selected from H or —C₁-C₄ alkyl;
   each v is independently 1, 2, 3, 4, 5 or 6;
L³ is absent, —C₁-C₂₀ alkylene-, —C₁-C₂₀ alkylene-NR¹⁴—, —C(═O)—C₁-C₂₀ alkylene-, —(CH₂CH₂O)_{w}—CH₂CH₂—, —C(═O)—(CH₂CH₂O)—CH₂CH₂—, —(CH₂CH₂NR¹⁴)_{w}—CH₂CH₂—, —C(═O)NR¹⁴—(CH₂CH₂NR¹⁴)_{w}—CH₂CH₂—, —C(═O)—NH—O—(CH₂)_{w}—, or —C(═O)—(CH₂)_{w}—O—NH—(CH₂)_{w}—C(═O)—; wherein each C₁-C₂₀ alkylene is optionally substituted with 1-3 substituents selected from the group consisting of —OH and —NH₂;
   each R⁴ is independently H or —C₁-C₆ alkyl;
   each w is independently 1, 2, 3, 4, 5 or 6;
L⁴ is absent, a natural or unnatural amino acid, or a peptide that is formed from two or more independently selected natural and unnatural amino acids, wherein when two or more amino acids are present then the N atom of the amide linking the amino acids is optionally substituted with —C₁-C₆ alkyl;
L⁵ is absent or -L⁷-L⁸-L⁹-;
   L⁷ is absent, —O—, —S—, —NH— or —NCH₃—;
   L⁸ is substituted or unsubstituted cycloalkylene, substituted or unsubstituted cycloalkenylene, or substituted or unsubstituted heterocycloalkylene;
   L⁹ is absent, —(CH₂)ₓ—, —C(═O)(CH₂)ₓ—, or —NR¹⁵(CH₂)ₓ—;
      x is 1, 2, 3, 4, 5 or 6;
   each R¹⁵ is independently selected from H or —C₁-C₆ alkyl; and
L⁶ is absent, —NH—, or —N(CH₃)—;
wherein if L⁶ is absent, L⁴ cannot be absent.

Embodiment 68. The compound of embodiment 67, wherein L² is absent, —C₁-C₆ alkylene, —C₁-C₆alkylene-NR¹³—, —C₁-C₂₀alkylene-NR¹³—C(═O)—, —NR¹³—C₁-C₂₀ alkylene-, —NR³—C₁-C₂₀ alkylene-NR¹⁶—, —C₁-C₆alkylene-C(═O)—, —C(═O)—C₁-C₆alkylene-, —C(═O)—C₁-C₆alkylene-NR¹³-, —C(═O)—NR¹³—C₁-C₂₀ alkylene-NR¹⁶—, or —C(═NH)NH—C₁-C₂₀ alkylene-NR¹³—, wherein each C₁-C₆ alkylene is optionally substituted with 1 —OH, —NH₂, and —COOH.

Embodiment 69. The compound of embodiment 67, wherein L² is —C₁-C₂₀ alkylene or —C₁-C₆ alkylene-NR¹³—, wherein R¹³ is H or —CH₃.

Embodiment 70. The compound of any one of embodiments 67-69, wherein L³ is —C₁-C₆ alkylene-, —C₁-C₆ alkylene-NR¹⁴—, or —C(═O)—C₁-C₆ alkylene-, wherein the C₁-C₆ alkylene is optionally substituted with 1 —OH or —NH₂.

Embodiment 71. The compound of any one of embodiments 67-70, or a pharmaceutically acceptable salt thereof, wherein L⁴ is absent.

Embodiment 72. The compound of any one of embodiments 67-70, or a pharmaceutically acceptable salt thereof, wherein L⁴ is a natural amino acid, an unnatural amino acid, or peptide that is formed from two or more independently selected amino acids selected from the group consisting of alanine (Ala), 3-aminoalanine (2,3-diaminopropionic acid) (Dap), arginine (Arg), asparagine (Asn), aspartate (Asp), biphenylalanine (Bip), cysteine (Cys), cysteic acid (sulfoalanine), glutamine (Gln), glutamate (Glu), homoglutamic acid (Aad), aminopimelic acid (Apm), glycine (Gly), histidine (His), isoleucine (Ile), leucine (Leu), lysine (Lys), hydroxylysine (Hyl), ornithine (Orn), methionine (Met), phenylalanine (Phe), proline (Pro), hydroxyproline (Hyp), serine (Ser), homoserine (Hse), sarcosine (Sar), threonine (Thr), tryptophan (Trp), tyrosine (Tyr), and valine (Val);
wherein when L⁴ is 3-aminoalanine, lysine (Lys), or ornithine (Orn), the N atom which is not part of the linker backbone can be substituted with

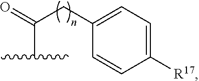

wherein R¹⁷ is —CH₃ or —I and n is 1, 2, or 3; and
wherein when L⁴ is aspartic acid or glutamic acid, the carbon atom of the carboxyl side NO₂ chain can be substituted with

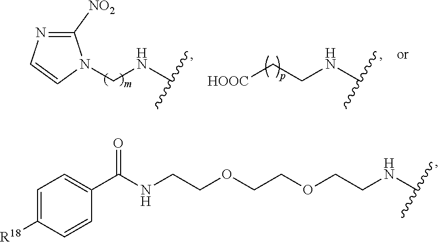

wherein m is 1, 2, or 3; p is 1, 2, 3, 4, 5, 6, 7, or 8; and R¹⁸ is —CH₃, —OCH₃, or —I; and
wherein when two or more amino acids are present then the N atom of the amide linking the amino acids is optionally substituted with —CH₃.

Embodiment 73. The compound of any one of embodiments 67-70, or a pharmaceutically acceptable salt thereof, wherein L⁴ is ornithine, lysine, glutamic acid, biphenylalanine (Bip), cysteic acid (sulfo-D-alanine), 3-amino alanine (2,3-diaminopropionic acid) (Dap), sarcosine, (4-(p-tolyl)butanoyl)-D-lysine, N5-(3-(2-nitro-1H-imidazol-1-yl)propyl)-L-glutamine, homoglutamic acid (Aad) or aminopimelic acid (Apm).

Embodiment 74. The compound of any one of embodiments 67-70, or a pharmaceutically acceptable salt thereof, wherein L⁴ is Sar-Sar-Sar.

Embodiment 75. The compound of any one of embodiments 67-70, or a pharmaceutically acceptable salt thereof, wherein L⁵ is absent.

Embodiment 76. The compound of any one of embodiments 67-74, or a pharmaceutically acceptable salt thereof, wherein L⁵ is -L⁷-L⁸-L⁹-;
L⁷ is absent, —NH—, or —NCH₃—;
   L⁸ is substituted or unsubstituted cycloalkenylene or substituted or unsubstituted heterocycloalkylene;
   L⁹ is absent, —C(═O)(CH₂)ₓ—, or —NR¹⁵(CH₂)ₓ—;
      x is 1, 2, 3, 4, 5 or 6; and
   each R¹⁵ is independently selected from H or —CH₃.

Embodiment 77. The compound of any one of embodiments 67-76, or a pharmaceutically acceptable salt thereof, wherein L⁸ is

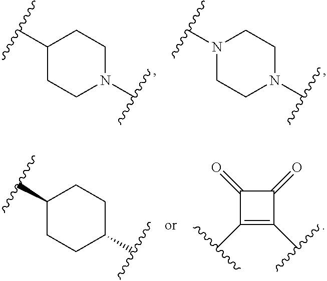 [SMILES: *C1CCN(*)CC1 ; *N1CCN(*)CC1 ; *[C@H]1CC[C@H](*)CC1 ; *c1c(*)c(=O)c1=O]

Embodiment 78. The compound of any one of embodiments 67-74, or a pharmaceutically acceptable salt thereof, wherein L⁵ is

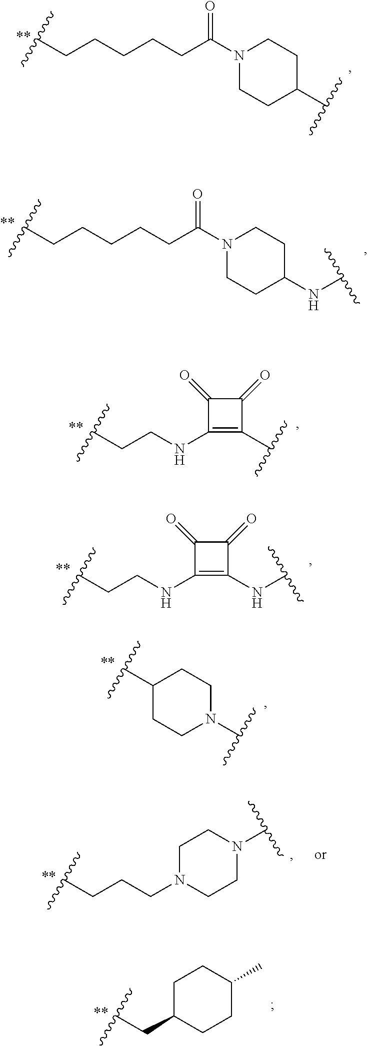

wherein denotes the attachment point of U to L.

Embodiment 79. The compound of any one of embodiments 67-78, or a pharmaceutically acceptable salt thereof, wherein L⁶ is —NH— or —N(CH₃)—.

Embodiment 80. The compound of any one of embodiments 1-67, wherein L of R^{a}-L- is:

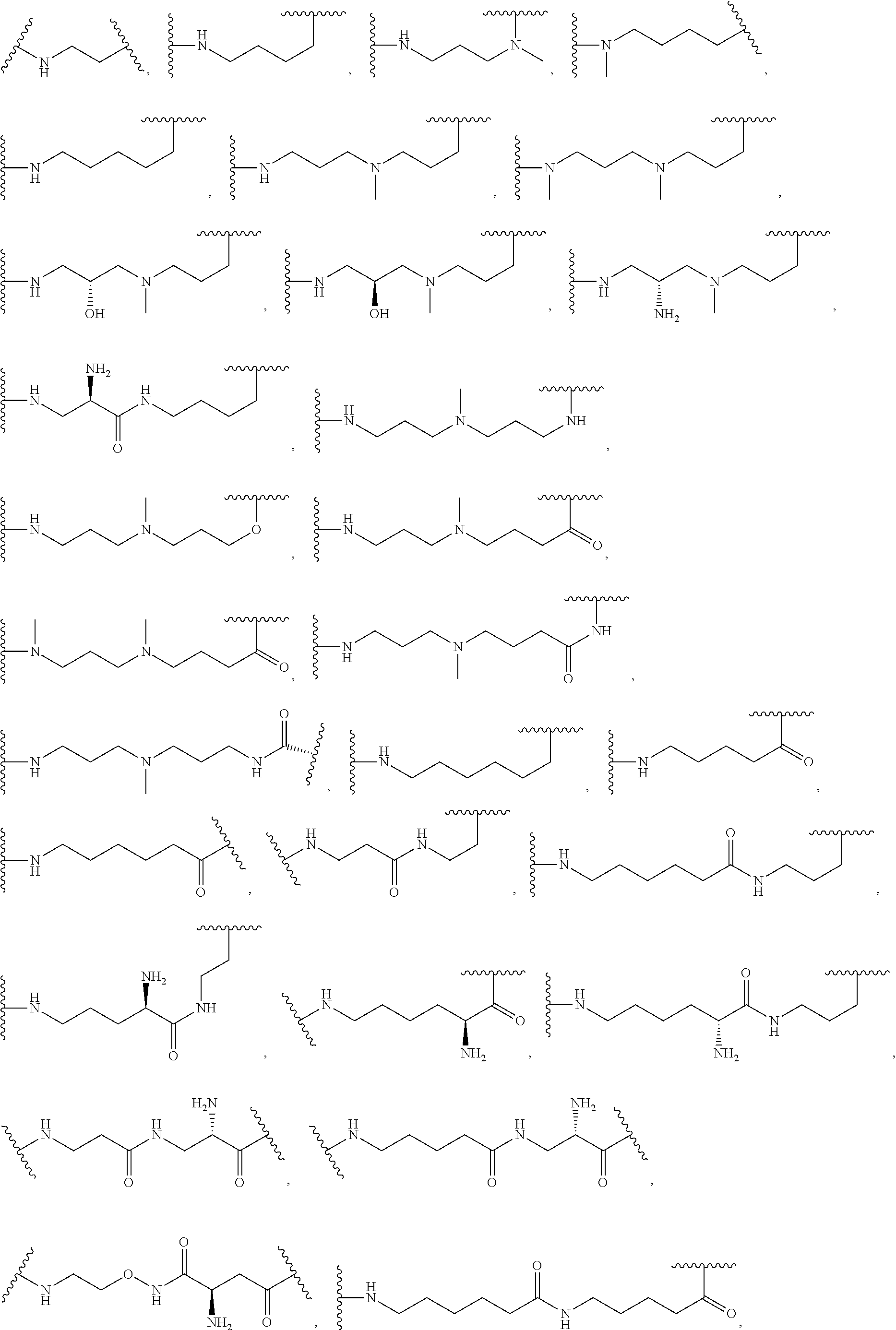

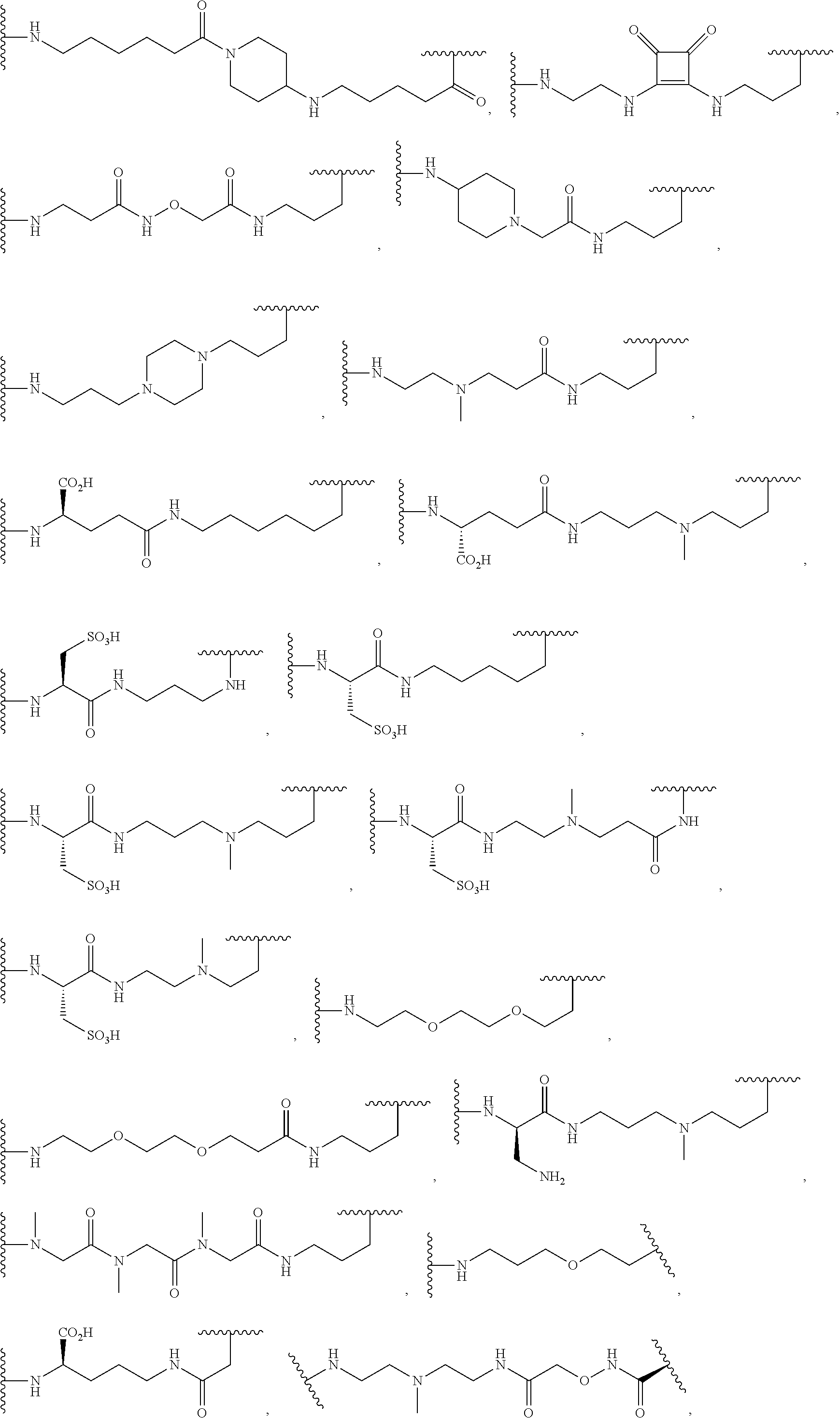

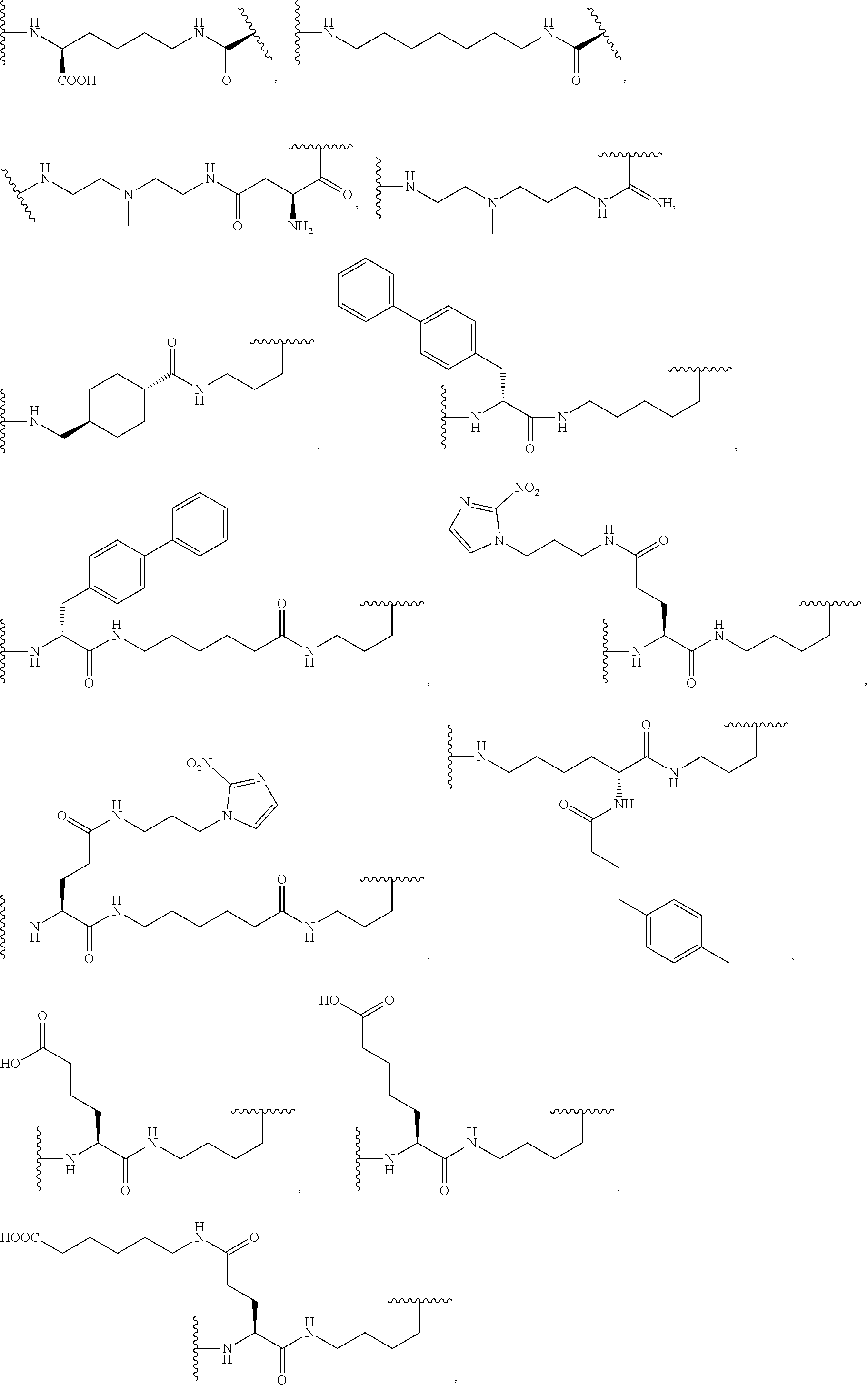

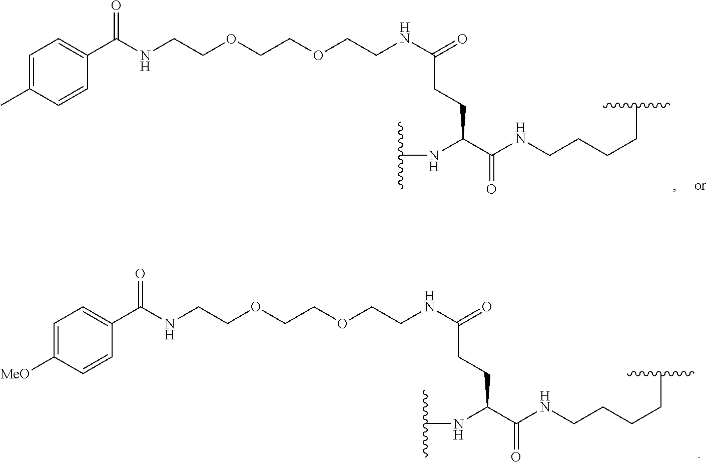 [SMILES: *CCCCNC(=O)[C@H](CCC(=O)NCCOCCOCCNC(=O)c1ccc(C)cc1)N* ; *CCCCNC(=O)[C@H](CCC(=O)NCCOCCOCCNC(=O)c1ccc(OC)cc1)N*]

Embodiment 81. The compound of any one of embodiments 1-67, wherein L of R^{a}-L- is:

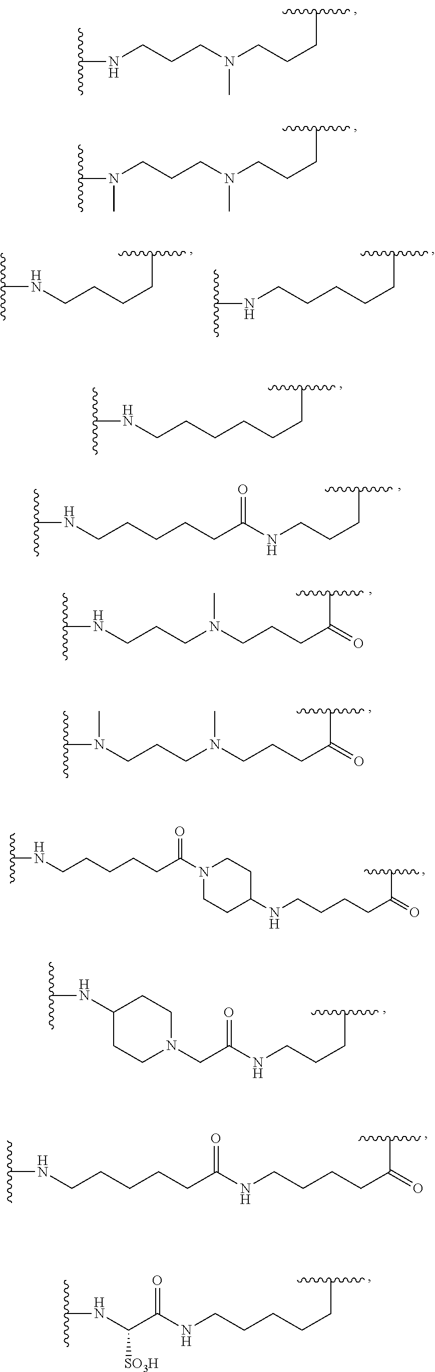

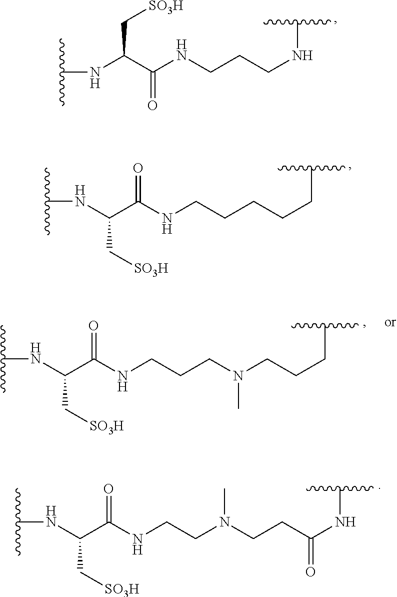 [SMILES: *NCCCNC(=O)[C@H](CS(=O)(=O)O)N* ; *CCCCCNC(=O)[C@H](CS(=O)(=O)O)N* ; *CCCN(C)CCCNC(=O)[C@H](CS(=O)(=O)O)N* ; *NC(=O)CCN(C)CCNC(=O)[C@H](CS(=O)(=O)O)N*]

Embodiment 82. The compound of any one of embodiments 1-67, wherein L of R^{a}-L- is:

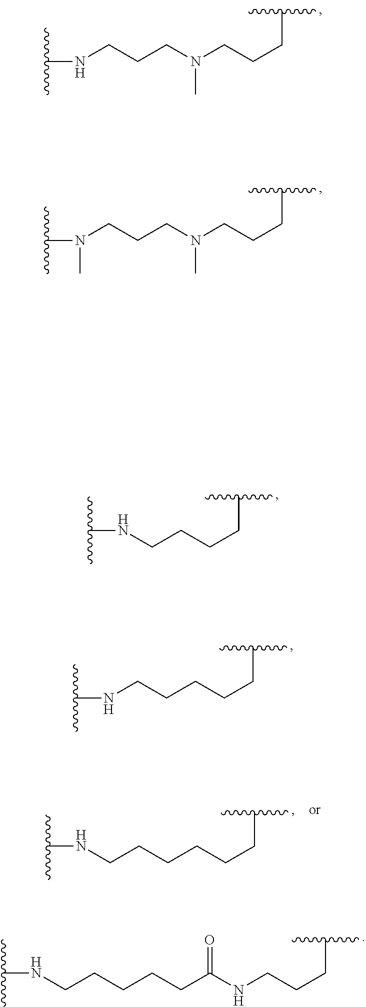 [SMILES: *CCCN(C)CCCN* ; *CCCN(C)CCCN(*)C ; *CCCCN* ; *CCCCCN* ; *CCCCCCN* ; *CCCNC(=O)CCCCCN*]

Embodiment 83. The compound of any one of embodiments 1-67, wherein L is:

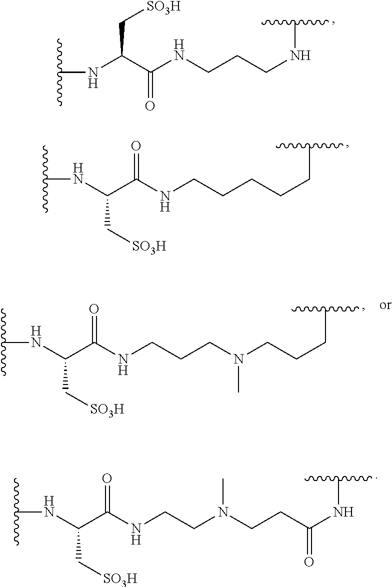

Embodiment 84. The compound of any one of embodiments 1-61, wherein R^{a}-L- is

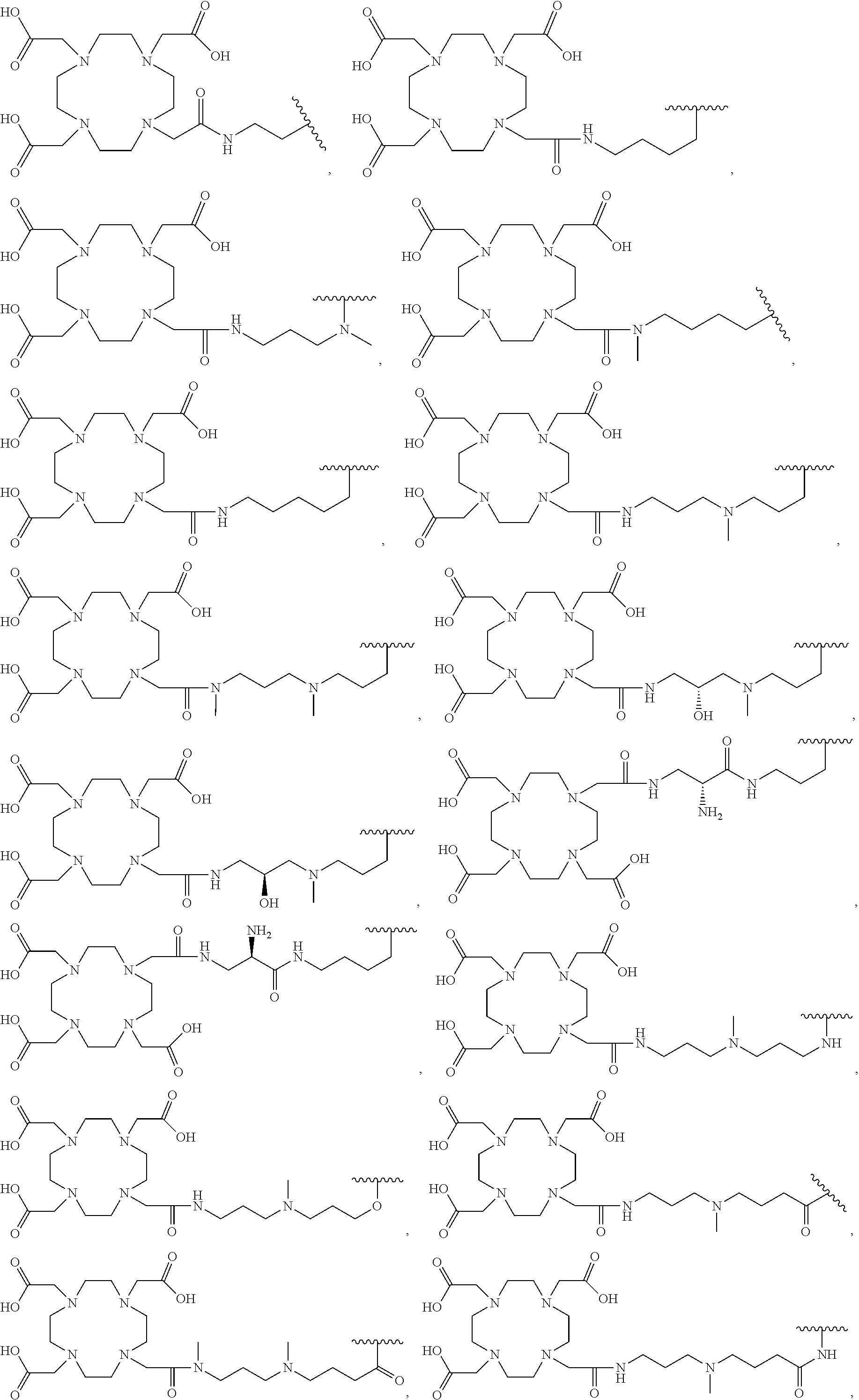

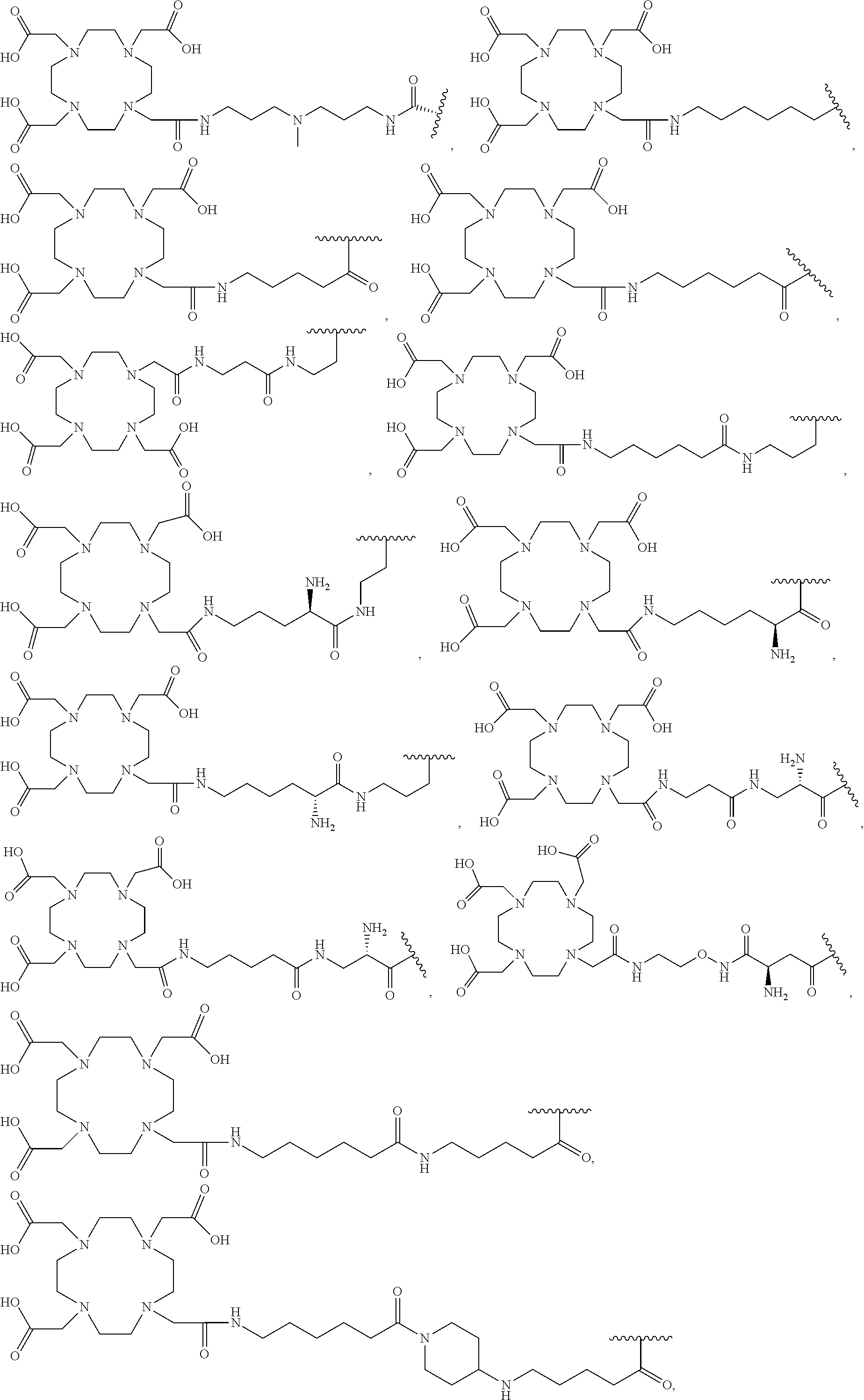

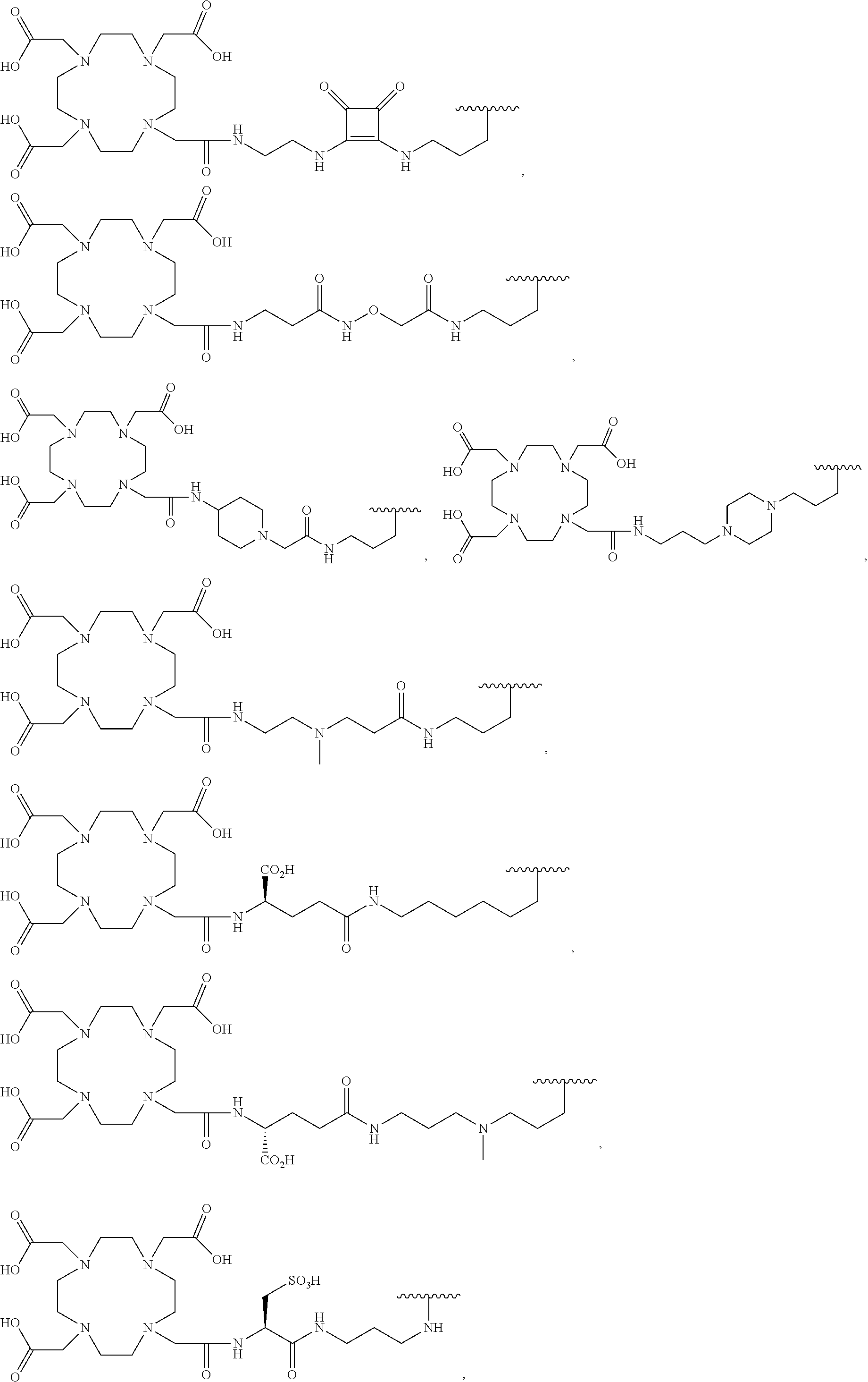

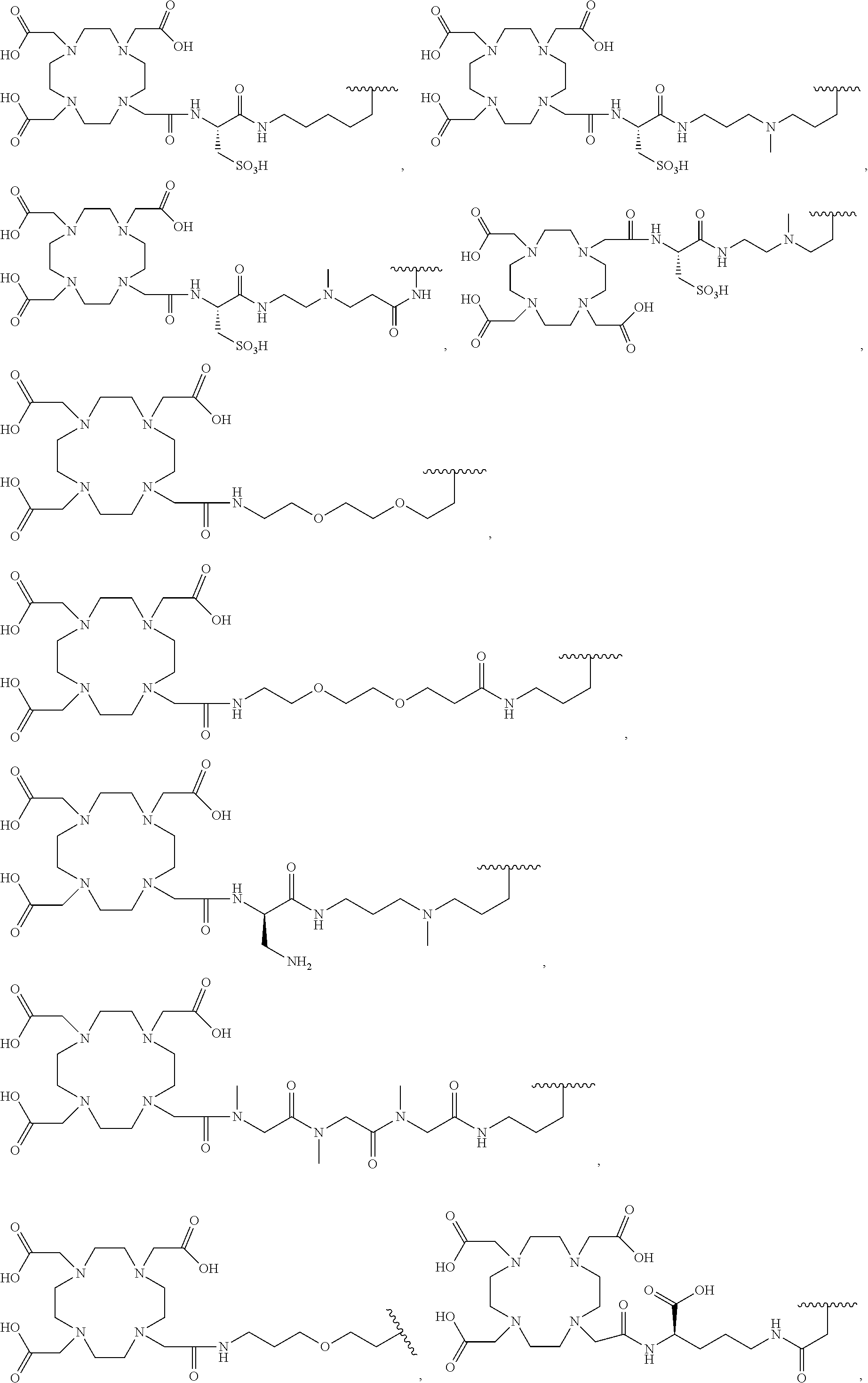

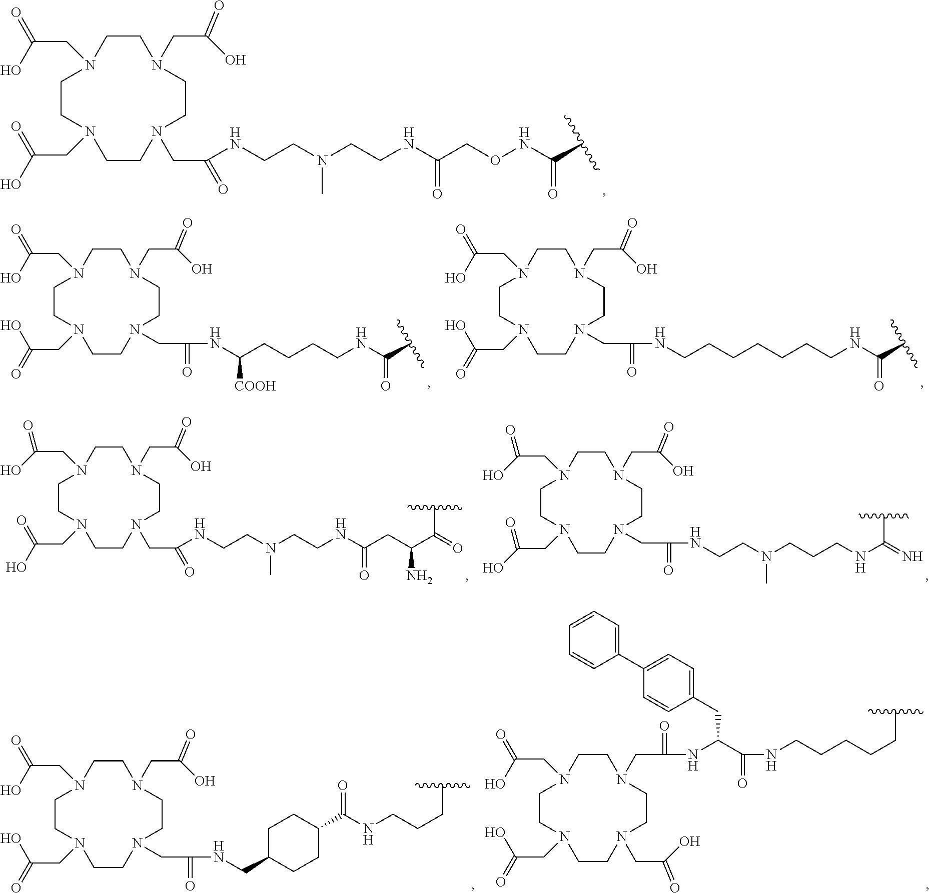

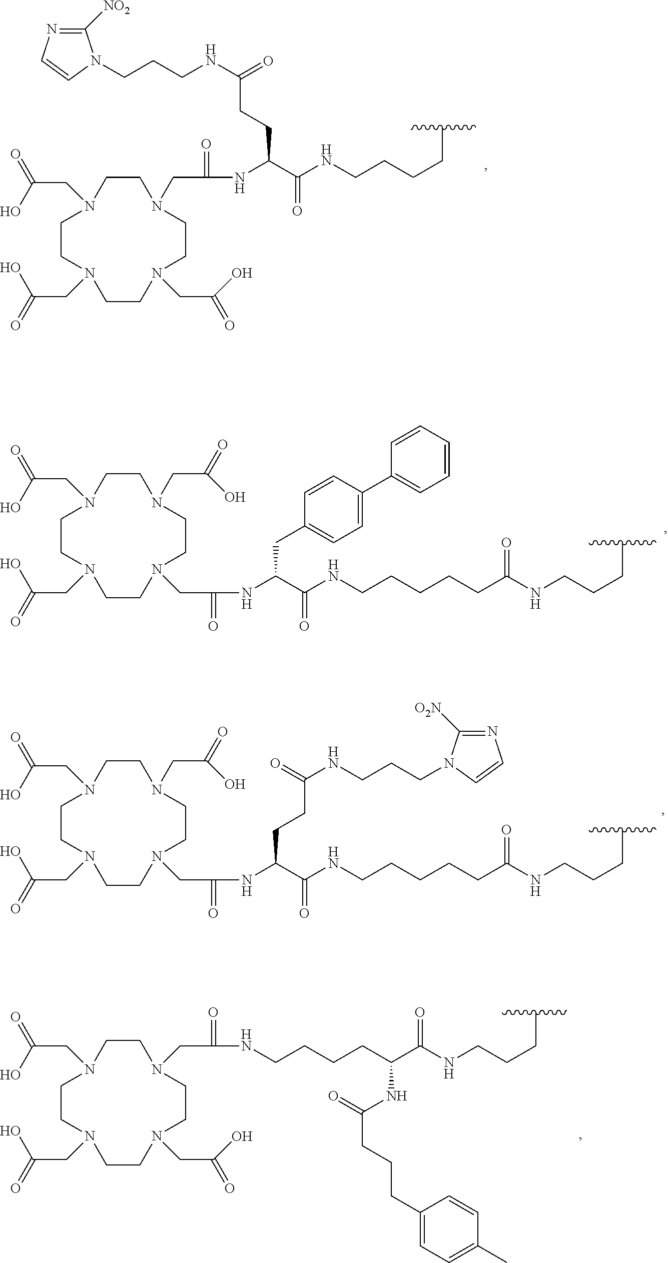 [SMILES: *CCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)CCCCCNC(=O)[C@@H](Cc1ccc(-c2ccccc2)cc1)NC(=O)CN1CCN(CC(=O)O)CCN(CC(=O)O)CCN(CC(=O)O)CC1 ; *CCCNC(=O)CCCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)[C@@H](CCCCNC(=O)CN1CCN(CC(=O)O)CCN(CC(=O)O)CCN(CC(=O)O)CC1)NC(=O)CCCc1ccc(C)cc1]

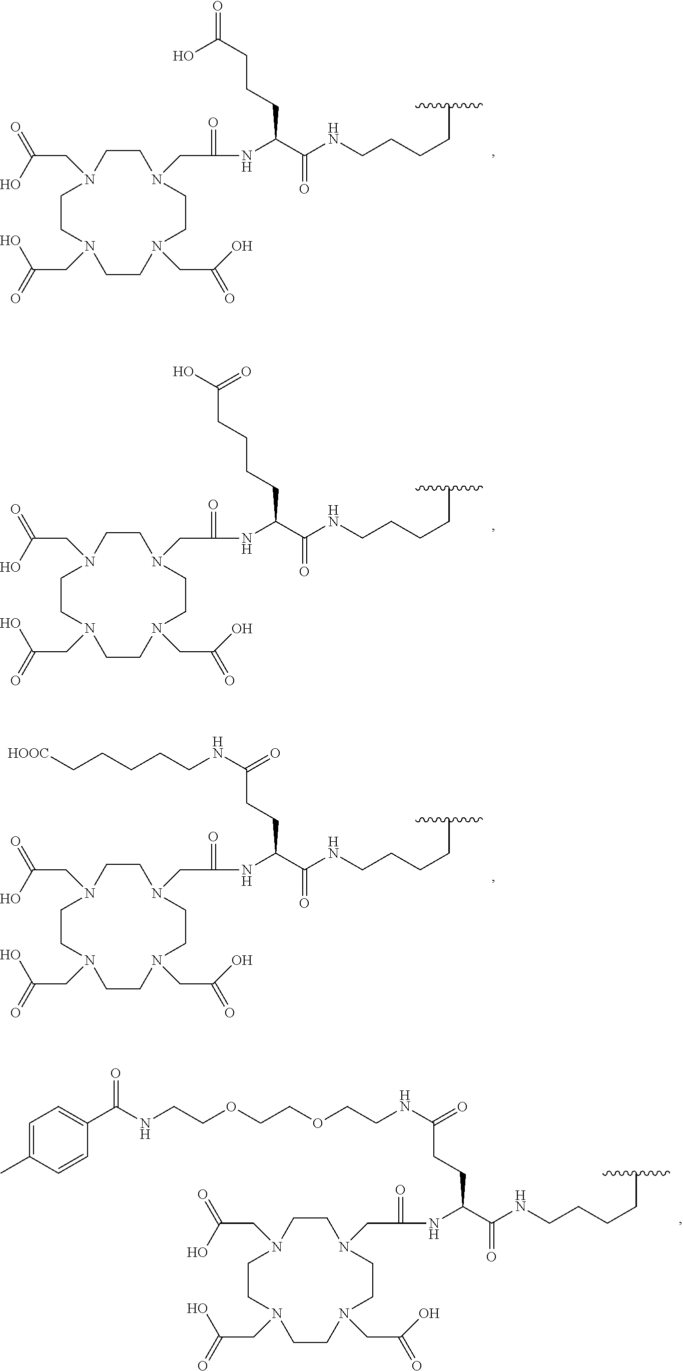 [SMILES: *CCCCNC(=O)[C@H](CCCC(=O)O)NC(=O)CN1CCN(CC(=O)O)CCN(CC(=O)O)CCN(CC(=O)O)CC1 ; *CCCCNC(=O)[C@H](CCCCC(=O)O)NC(=O)CN1CCN(CC(=O)O)CCN(CC(=O)O)CCN(CC(=O)O)CC1 ; *CCCCNC(=O)[C@H](CCC(=O)NCCCCCC(=O)O)NC(=O)CN1CCN(CC(=O)O)CCN(CC(=O)O)CCN(CC(=O)O)CC1 ; *CCCCNC(=O)[C@H](CCC(=O)NCCOCCOCCNC(=O)c1ccc(C)cc1)NC(=O)CN1CCN(CC(=O)O)CCN(CC(=O)O)CCN(CC(=O)O)CC1]

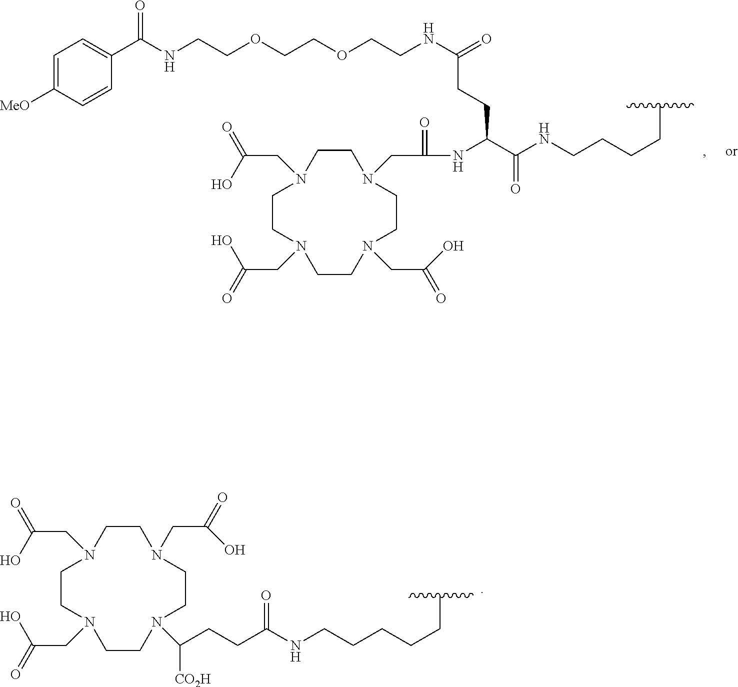 [SMILES: *CCCCNC(=O)[C@H](CCC(=O)NCCOCCOCCNC(=O)c1ccc(OC)cc1)NC(=O)CN1CCN(CC(=O)O)CCN(CC(=O)O)CCN(CC(=O)O)CC1 ; *CCCCCNC(=O)CCC(C(=O)O)N1CCN(CC(=O)O)CCN(CC(=O)O)CCN(CC(=O)O)CC1]

Embodiment 85. The compound of embodiment 1, or a pharmaceutically acceptable salt thereof, wherein the compound of Formula (A) has one of the following structures, or a pharmaceutically acceptable salt thereof.

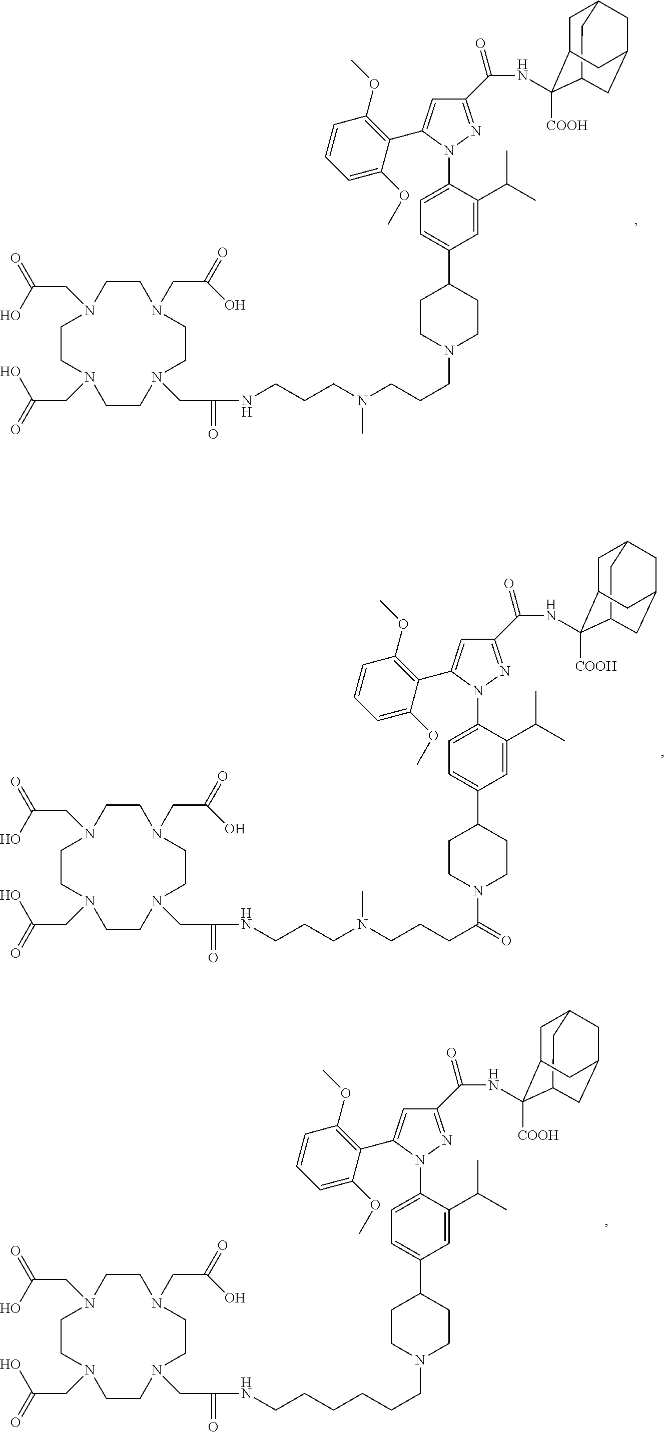 [SMILES: COc1cccc(OC)c1-c1cc(C(=O)NC2(C(=O)O)C3CC4CC(C3)CC2C4)nn1-c1ccc(C2CCN(CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCCCNC(=O)CN3CCN(CC(=O)O)CCN(CC(=O)O)CCN(CC(=O)O)CC3)CC2)cc1C(C)C]

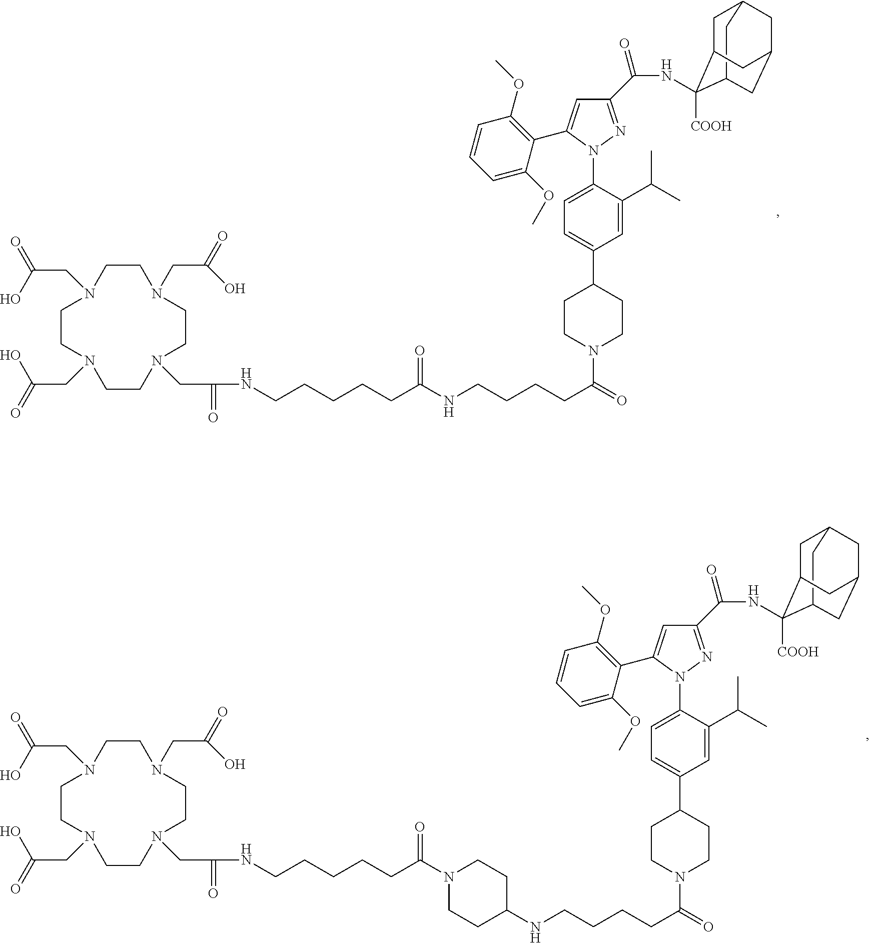 [SMILES: COc1cccc(OC)c1-c1cc(C(=O)NC2(C(=O)O)C3CC4CC(C3)CC2C4)nn1-c1ccc(C2CCN(C(=O)CCCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCCNC3CCN(C(=O)CCCCCNC(=O)CN4CCN(CC(=O)O)CCN(CC(=O)O)CCN(CC(=O)O)CC4)CC3)CC2)cc1C(C)C]

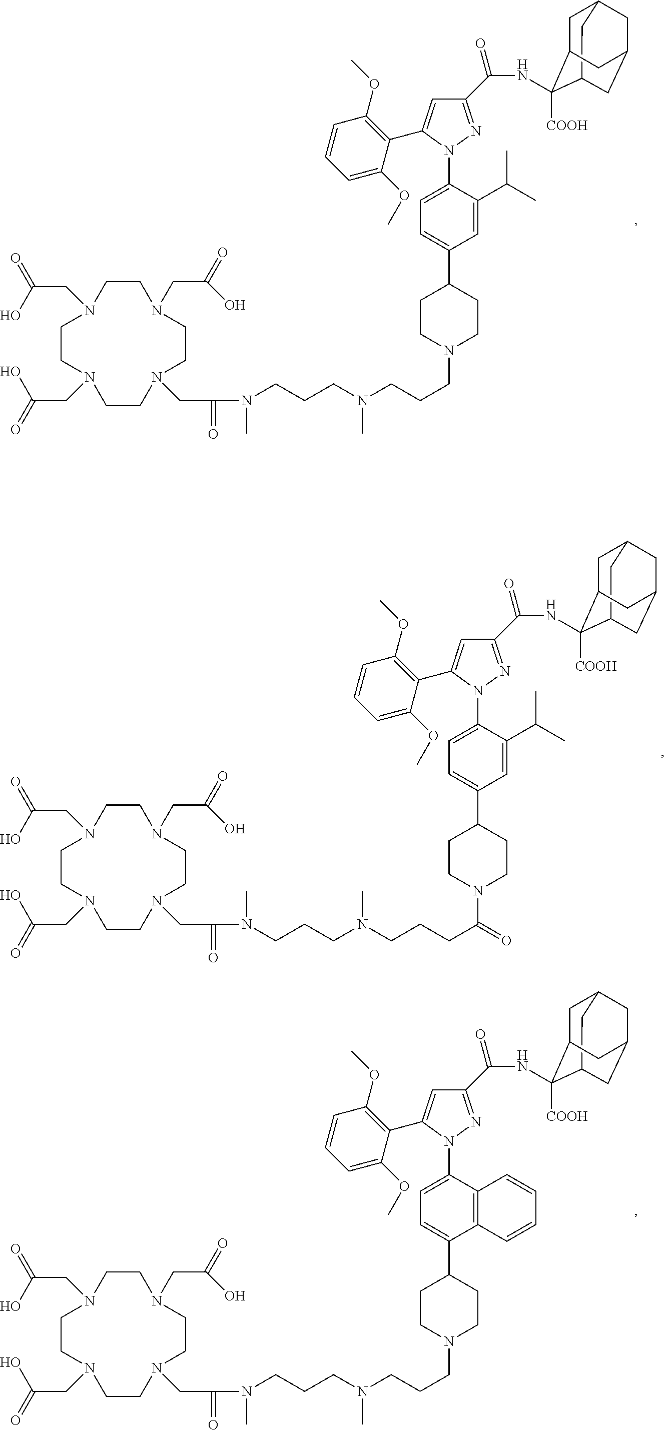 [SMILES: COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCN(C)CCCN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)c2ccccc12]

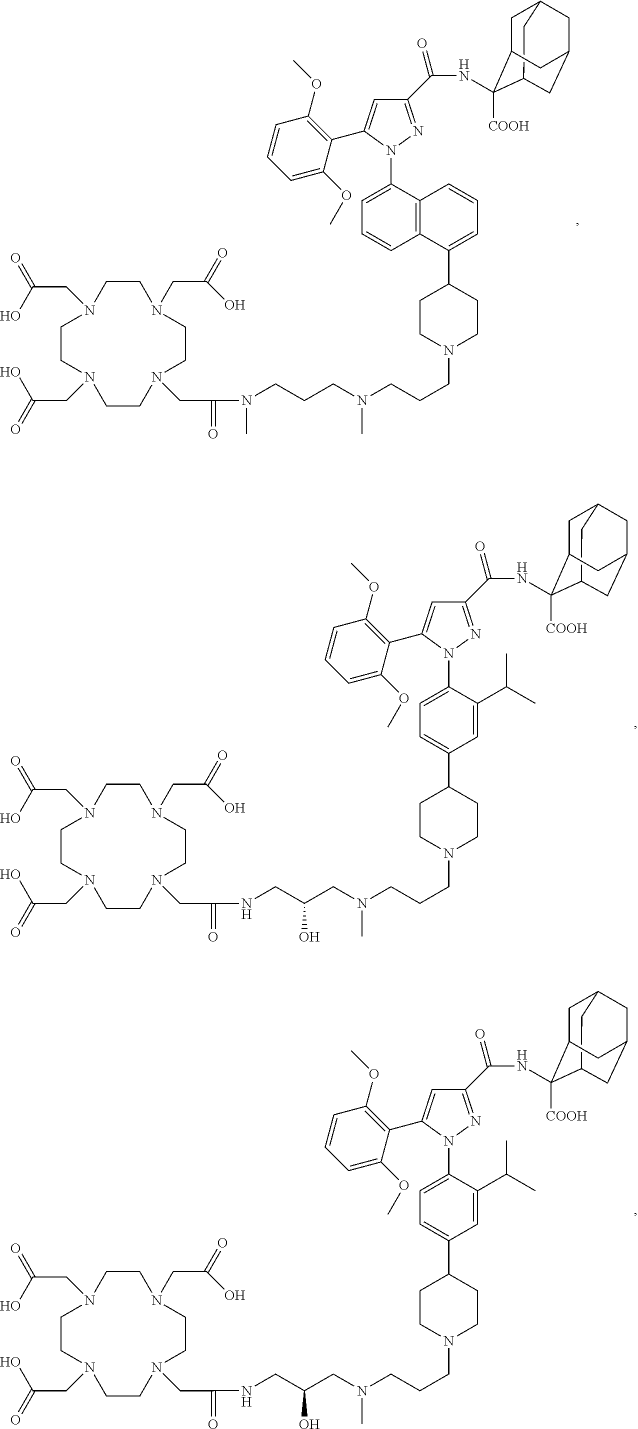 [SMILES: COc1cccc(OC)c1-c1cc(C(=O)NC2(C(=O)O)C3CC4CC(C3)CC2C4)nn1-c1cccc2c(C3CCN(CCCN(C)CCCN(C)C(=O)CN4CCN(CC(=O)O)CCN(CC(=O)O)CCN(CC(=O)O)CC4)CC3)cccc12 ; COc1cccc(OC)c1-c1cc(C(=O)NC2(C(=O)O)C3CC4CC(C3)CC2C4)nn1-c1ccc(C2CCN(CCCN(C)C[C@H](O)CNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCN(C)C[C@@H](O)CNC(=O)CN3CCN(CC(=O)O)CCN(CC(=O)O)CCN(CC(=O)O)CC3)CC2)cc1C(C)C]

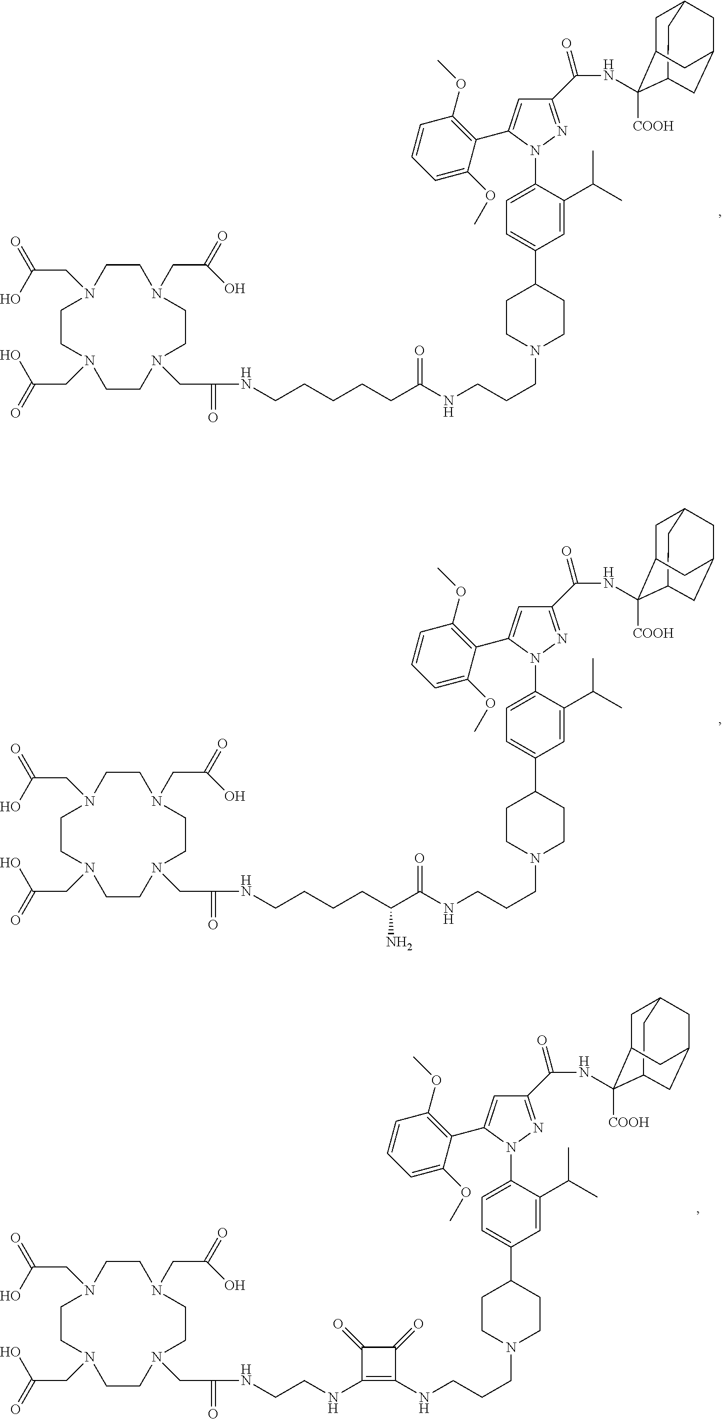 [SMILES: COc1cccc(OC)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)[C@H](N)CCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNc3c(NCCNC(=O)CN4CCN(CC(=O)O)CCN(CC(=O)O)CCN(CC(=O)O)CC4)c(=O)c3=O)CC2)cc1C(C)C]

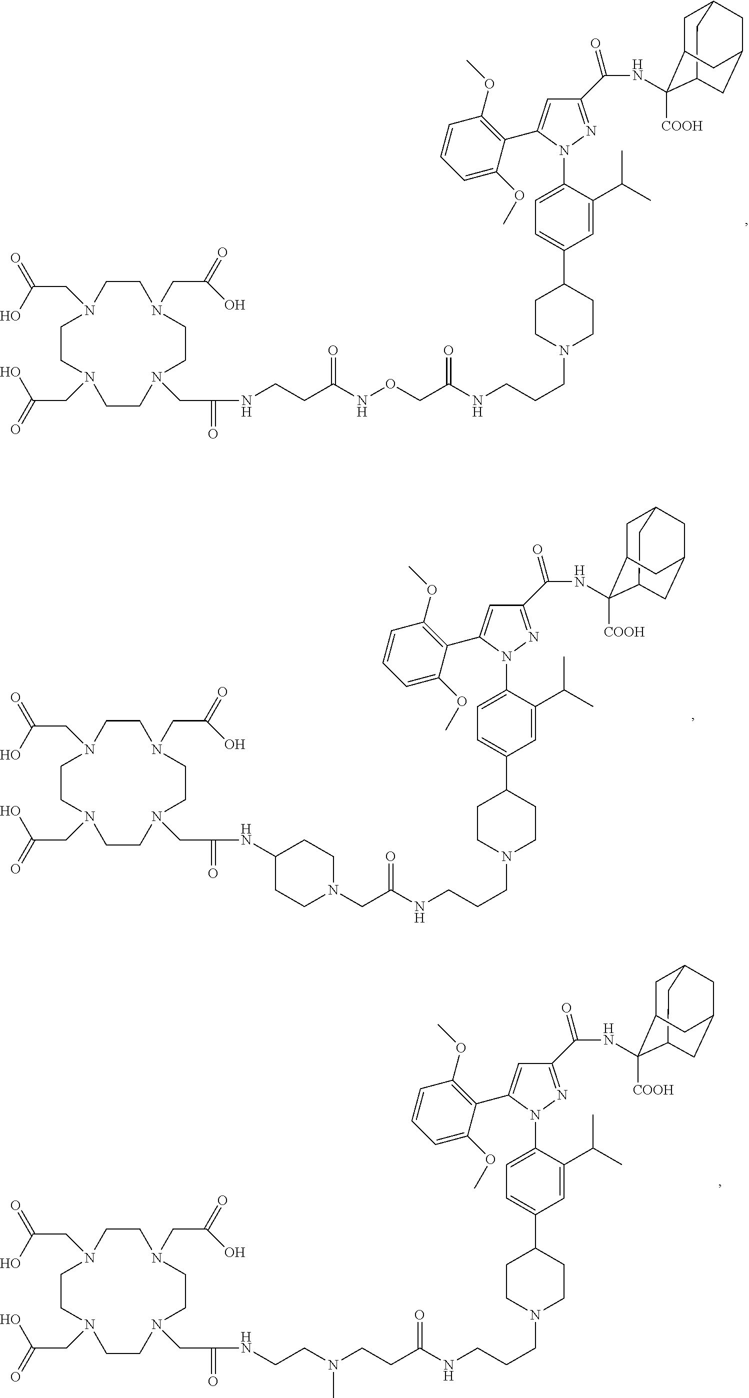 [SMILES: COc1cccc(OC)c1-c1cc(C(=O)NC2(C(=O)O)C3CC4CC(C3)CC2C4)nn1-c1ccc(C2CCN(CCCNC(=O)CONC(=O)CCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CN3CCC(NC(=O)CN4CCN(CC(=O)O)CCN(CC(=O)O)CCN(CC(=O)O)CC4)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CCN(C)CCNC(=O)CN3CCN(CC(=O)O)CCN(CC(=O)O)CCN(CC(=O)O)CC3)CC2)cc1C(C)C]

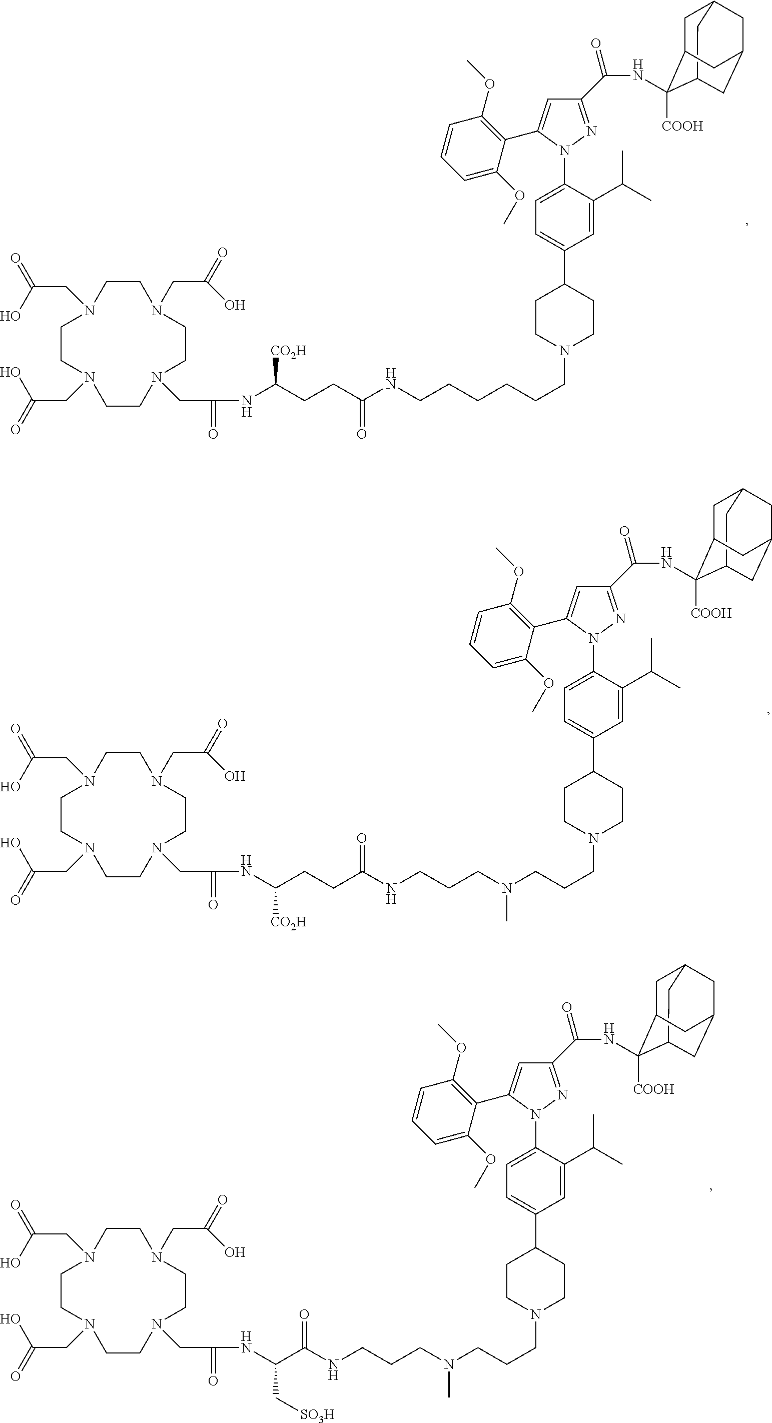 [SMILES: COc1cccc(OC)c1-c1cc(C(=O)NC2(C(=O)O)C3CC4CC(C3)CC2C4)nn1-c1ccc(C2CCN(CCCCCCNC(=O)CC[C@@H](NC(=O)CN3CCN(CC(=O)O)CCN(CC(=O)O)CCN(CC(=O)O)CC3)C(=O)O)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)CC[C@@H](NC(=O)CN3CCN(CC(=O)O)CCN(CC(=O)O)CCN(CC(=O)O)CC3)C(=O)O)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C]

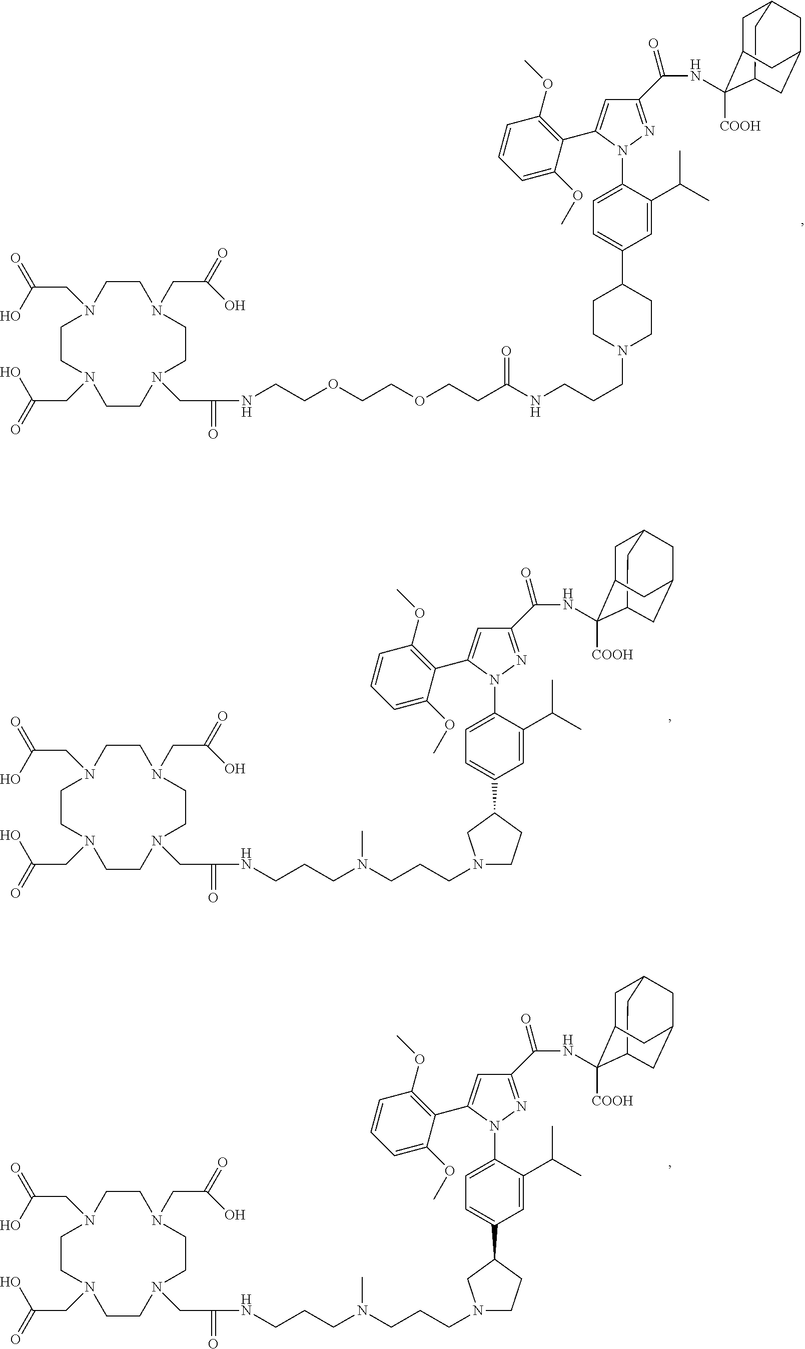 [SMILES: COc1cccc(OC)c1-c1cc(C(=O)NC2(C(=O)O)C3CC4CC(C3)CC2C4)nn1-c1ccc(C2CCN(CCCNC(=O)CCOCCOCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc([C@@H]2CCN(CCCN(C)CCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc([C@H]2CCN(CCCN(C)CCCNC(=O)CN3CCN(CC(=O)O)CCN(CC(=O)O)CCN(CC(=O)O)CC3)C2)cc1C(C)C]

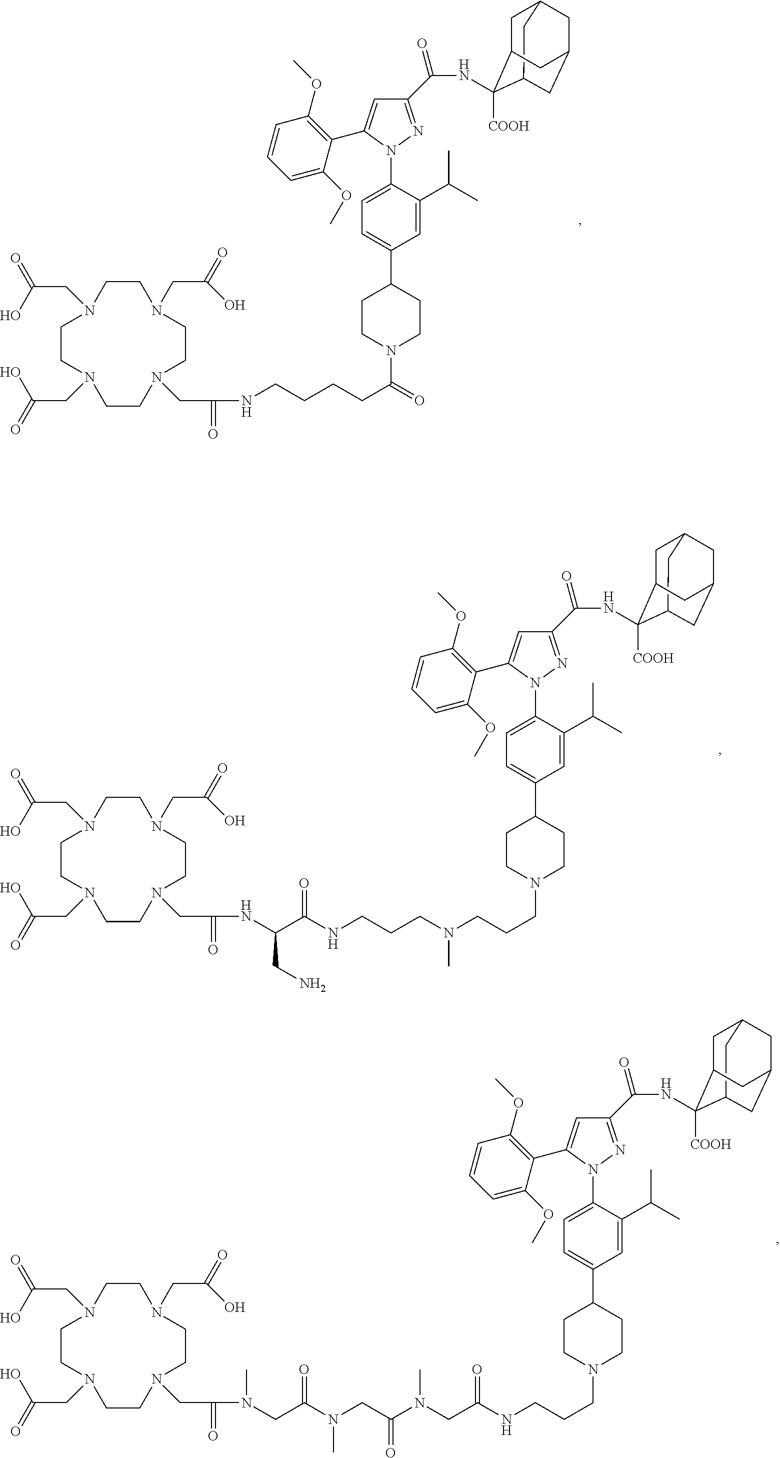 [SMILES: COc1cccc(OC)c1-c1cc(C(=O)NC2(C(=O)O)C3CC4CC(C3)CC2C4)nn1-c1ccc(C2CCN(C(=O)CCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)[C@@H](CN)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CN(C)C(=O)CN(C)C(=O)CN(C)C(=O)CN3CCN(CC(=O)O)CCN(CC(=O)O)CCN(CC(=O)O)CC3)CC2)cc1C(C)C]

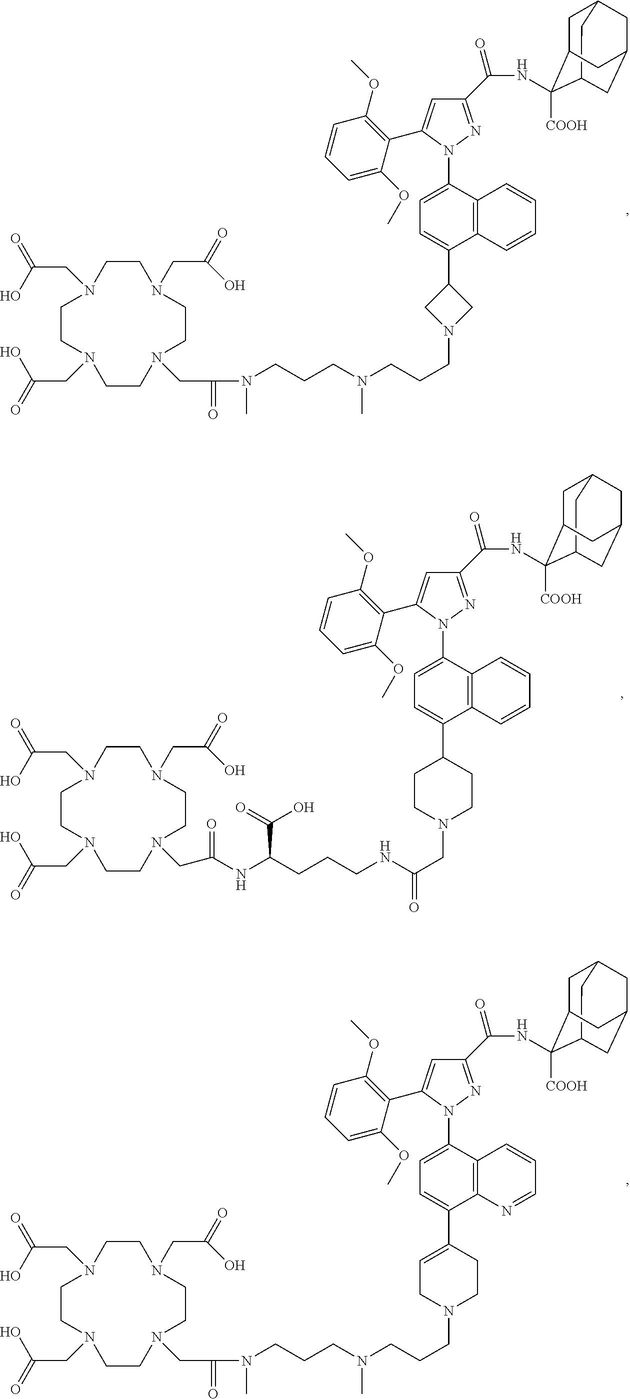 [SMILES: COc1cccc(OC)c1-c1cc(C(=O)NC2(C(=O)O)C3CC4CC(C3)CC2C4)nn1-c1ccc(C2CN(CCCN(C)CCCN(C)C(=O)CN3CCN(CC(=O)O)CCN(CC(=O)O)CCN(CC(=O)O)CC3)C2)c2ccccc12 ; COc1cccc(OC)c1-c1cc(C(=O)NC2(C(=O)O)C3CC4CC(C3)CC2C4)nn1-c1ccc(C2CCN(CC(=O)NCCC[C@@H](NC(=O)CN3CCN(CC(=O)O)CCN(CC(=O)O)CCN(CC(=O)O)CC3)C(=O)O)CC2)c2ccccc12 ; COc1cccc(OC)c1-c1cc(C(=O)NC2(C(=O)O)C3CC4CC(C3)CC2C4)nn1-c1ccc(C2=CCN(CCCN(C)CCCN(C)C(=O)CN3CCN(CC(=O)O)CCN(CC(=O)O)CCN(CC(=O)O)CC3)CC2)c2ncccc12]

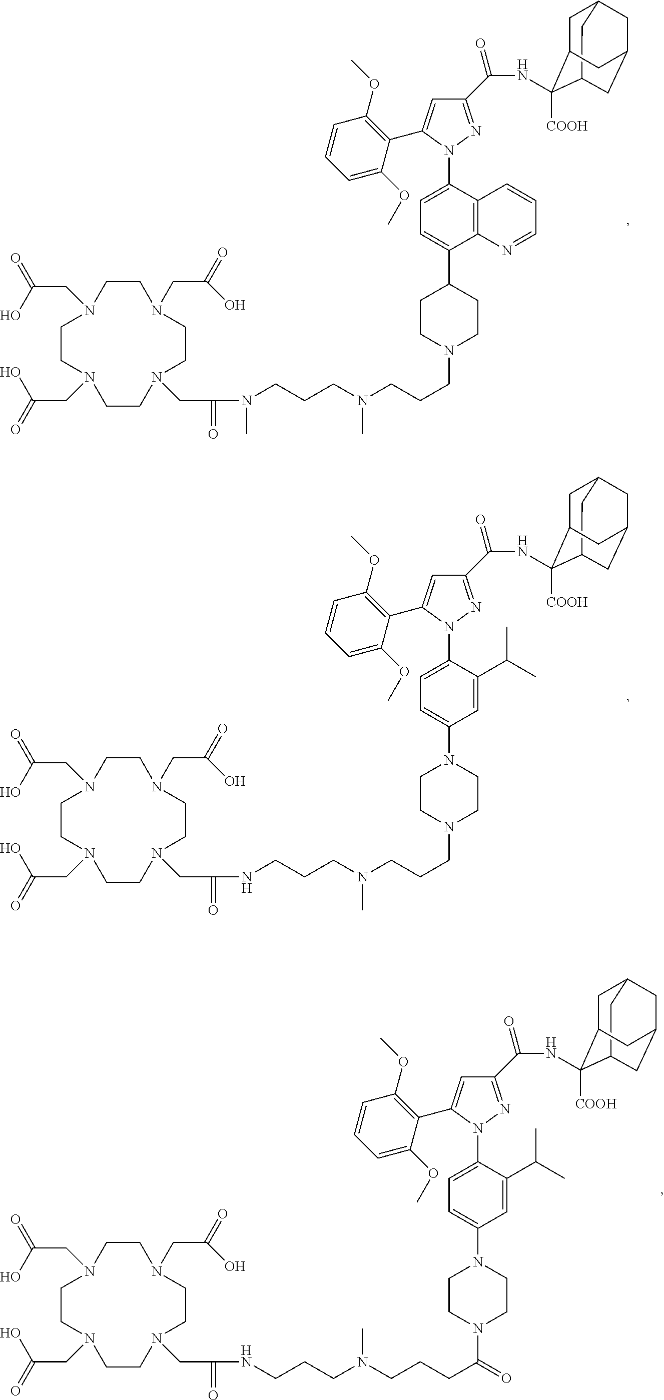 [SMILES: COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)c2ncccc12 ; COc1cccc(OC)c1-c1cc(C(=O)NC2(C(=O)O)C3CC4CC(C3)CC2C4)nn1-c1ccc(N2CCN(CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCN(C(=O)CCCN(C)CCCNC(=O)CN3CCN(CC(=O)O)CCN(CC(=O)O)CCN(CC(=O)O)CC3)CC2)cc1C(C)C]

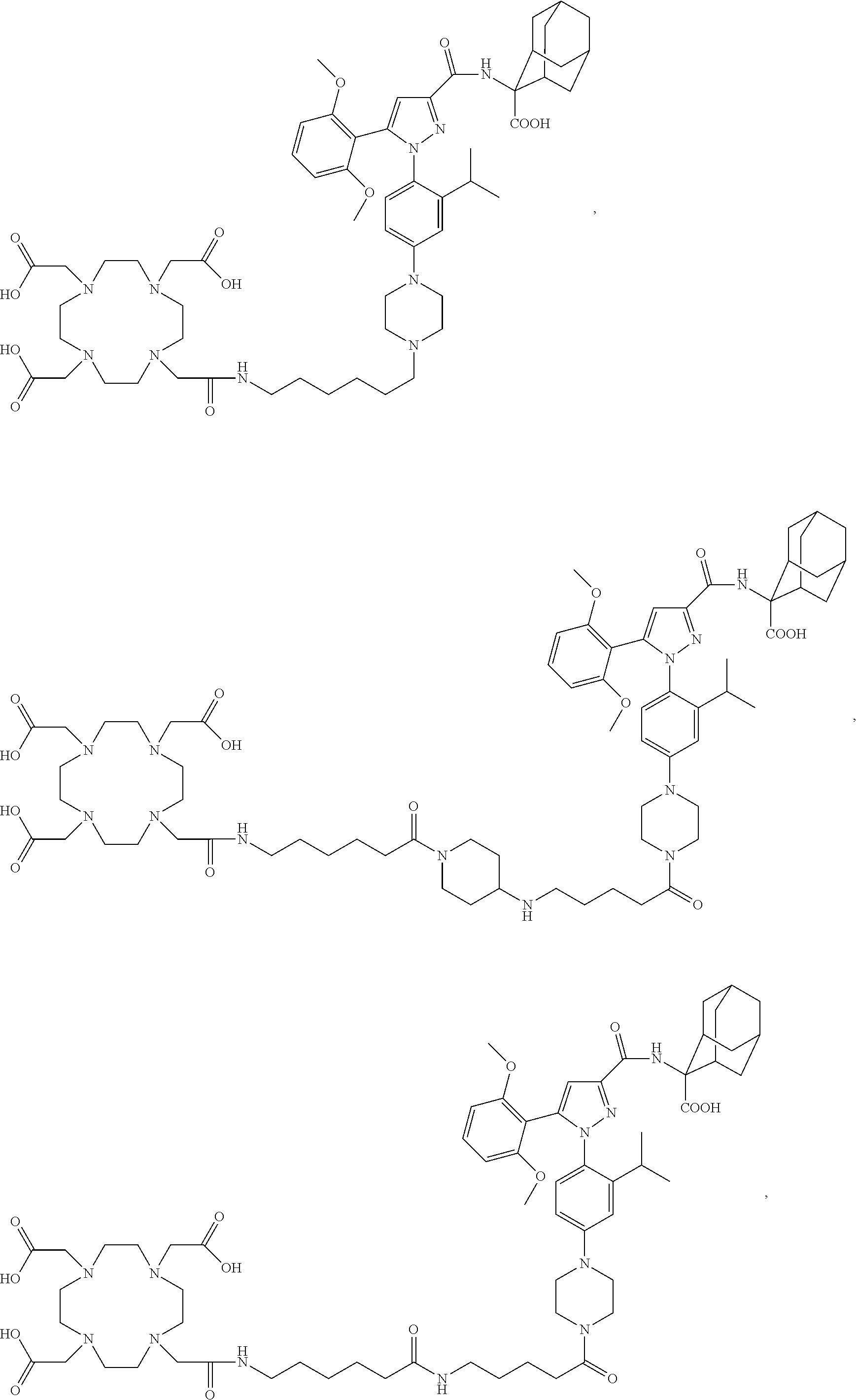 [SMILES: COc1cccc(OC)c1-c1cc(C(=O)NC2(C(=O)O)C3CC4CC(C3)CC2C4)nn1-c1ccc(N2CCN(CCCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCN(C(=O)CCCCNC3CCN(C(=O)CCCCCNC(=O)CN4CCN(CC(=O)O)CCN(CC(=O)O)CCN(CC(=O)O)CC4)CC3)CC2)cc1C(C)C ; COc1cccc(OC)c1-c1cc(C(=O)NC2(C(=O)O)C3CC4CC(C3)CC2C4)nn1-c1ccc(N2CCN(C(=O)CCCCNC(=O)CCCCCNC(=O)CN3CCN(CC(=O)O)CCN(CC(=O)O)CCN(CC(=O)O)CC3)CC2)cc1C(C)C]

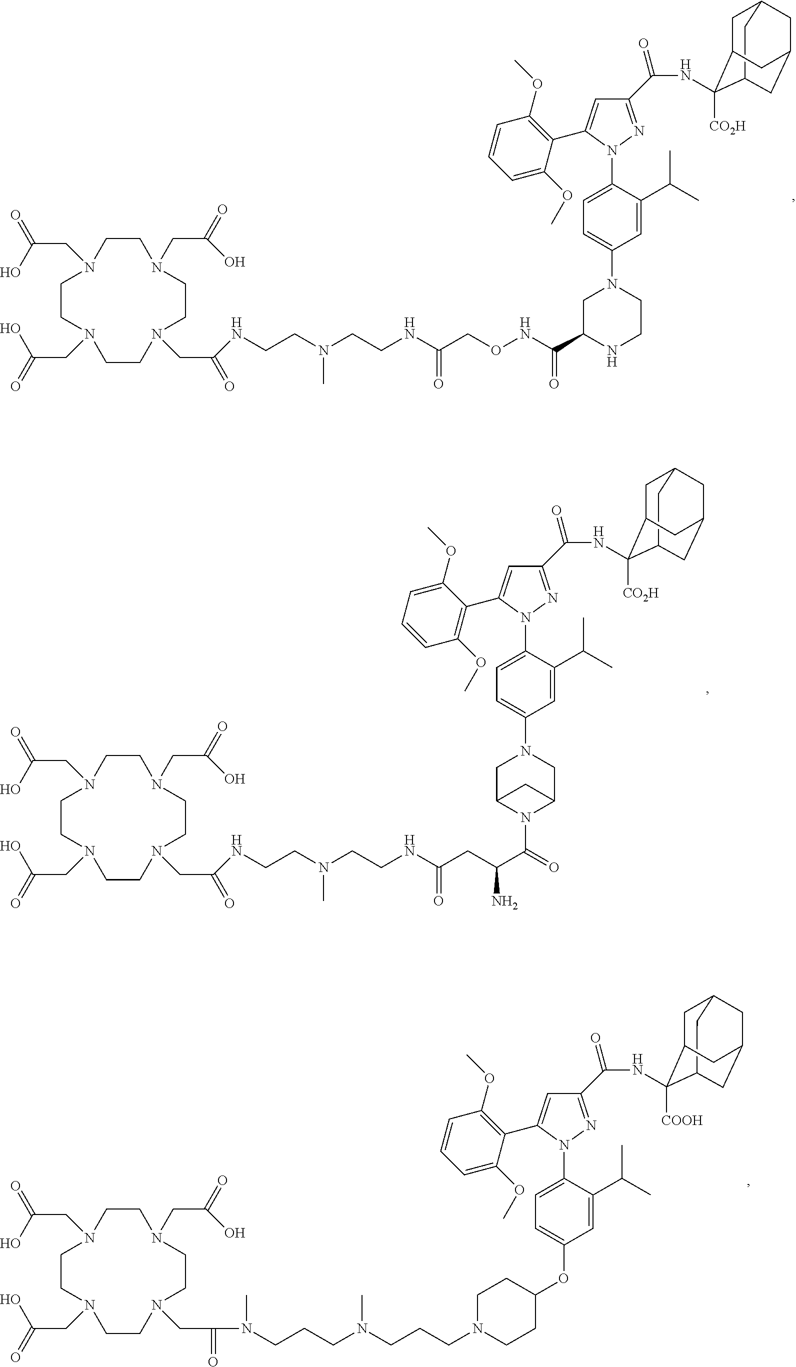 [SMILES: COc1cccc(OC)c1-c1cc(C(=O)NC2(C(=O)O)C3CC4CC(C3)CC2C4)nn1-c1ccc(N2CCN[C@@H](C(=O)NOCC(=O)NCCN(C)CCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(N2CC3CC(C2)N3C(=O)[C@@H](N)CC(=O)NCCN(C)CCNC(=O)CN2CCN(CC(=O)O)CCN(CC(=O)O)CCN(CC(=O)O)CC2)cc1C(C)C ; COc1cccc(OC)c1-c1cc(C(=O)NC2(C(=O)O)C3CC4CC(C3)CC2C4)nn1-c1ccc(OC2CCN(CCCN(C)CCCN(C)C(=O)CN3CCN(CC(=O)O)CCN(CC(=O)O)CCN(CC(=O)O)CC3)CC2)cc1C(C)C]

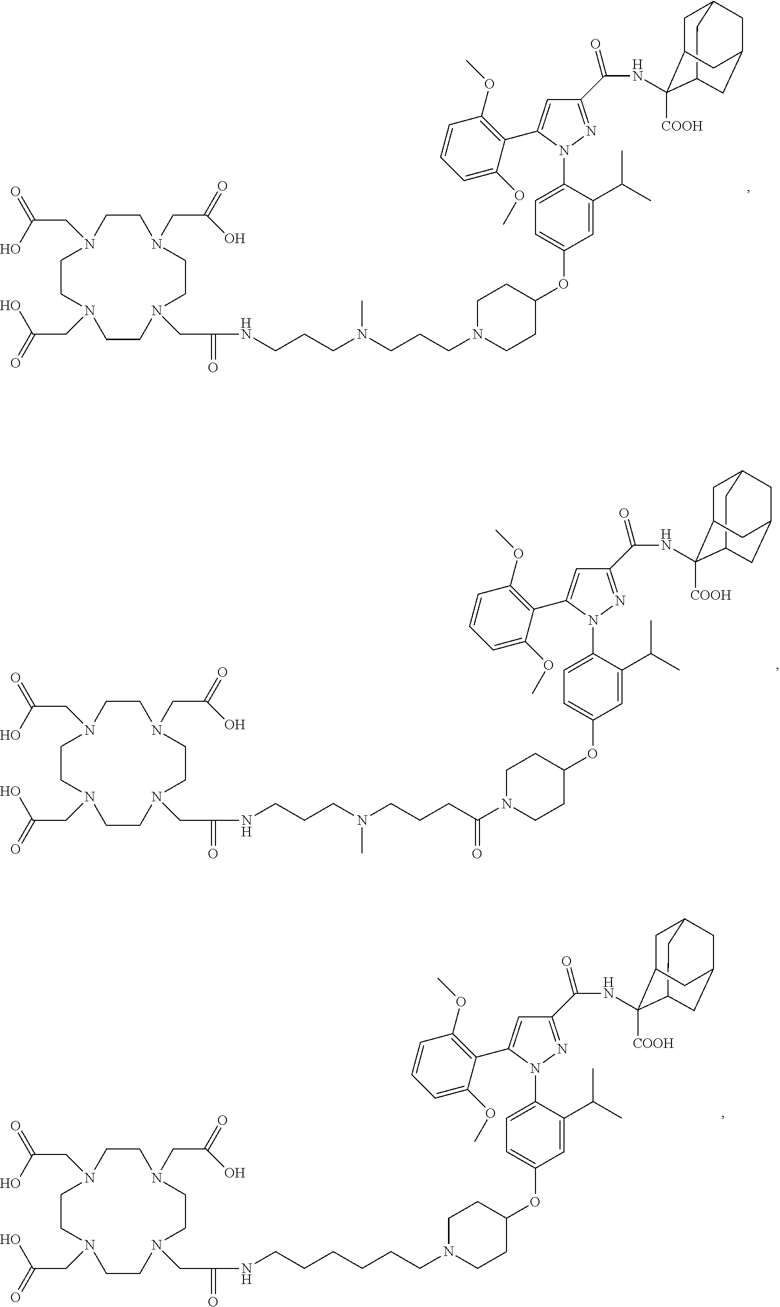 [SMILES: COc1cccc(OC)c1-c1cc(C(=O)NC2(C(=O)O)C3CC4CC(C3)CC2C4)nn1-c1ccc(OC2CCN(CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(C(=O)CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(CCCCCCNC(=O)CN3CCN(CC(=O)O)CCN(CC(=O)O)CCN(CC(=O)O)CC3)CC2)cc1C(C)C]

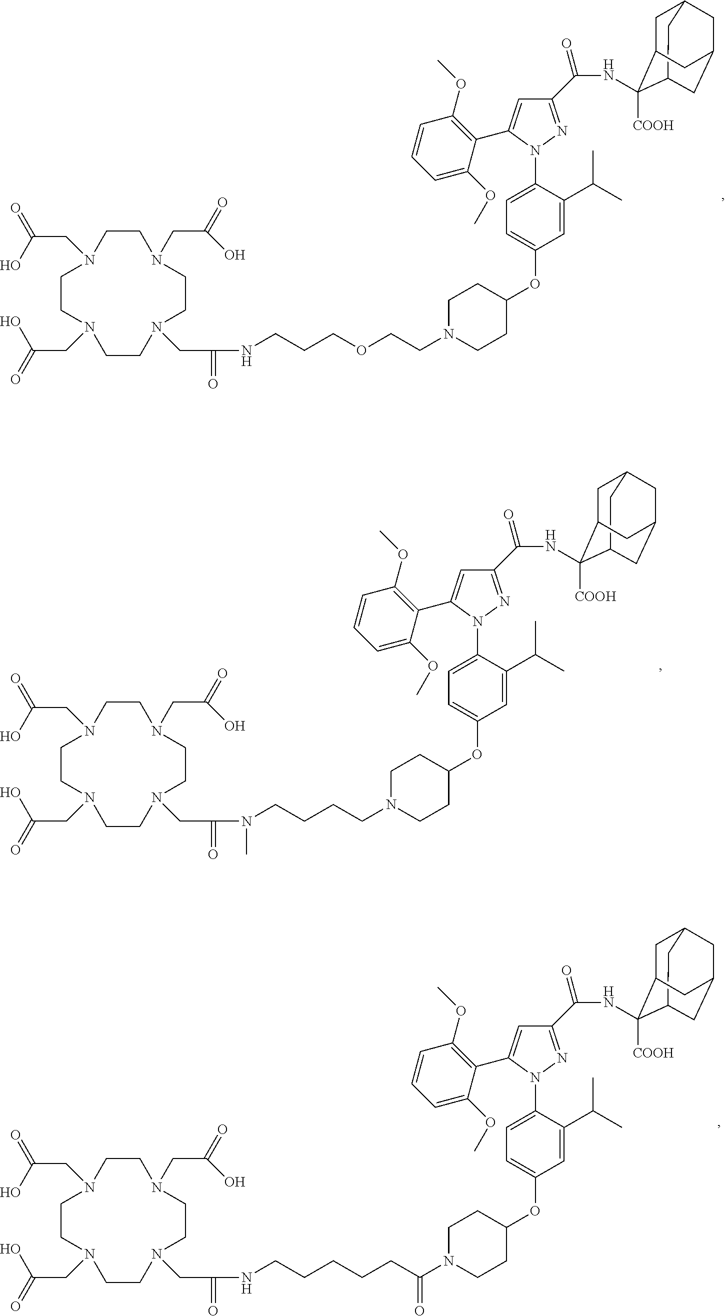 [SMILES: COc1cccc(OC)c1-c1cc(C(=O)NC2(C(=O)O)C3CC4CC(C3)CC2C4)nn1-c1ccc(OC2CCN(CCOCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(CCCCN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CCN(C(=O)CCCCCNC(=O)CN3CCN(CC(=O)O)CCN(CC(=O)O)CCN(CC(=O)O)CC3)CC2)cc1C(C)C]

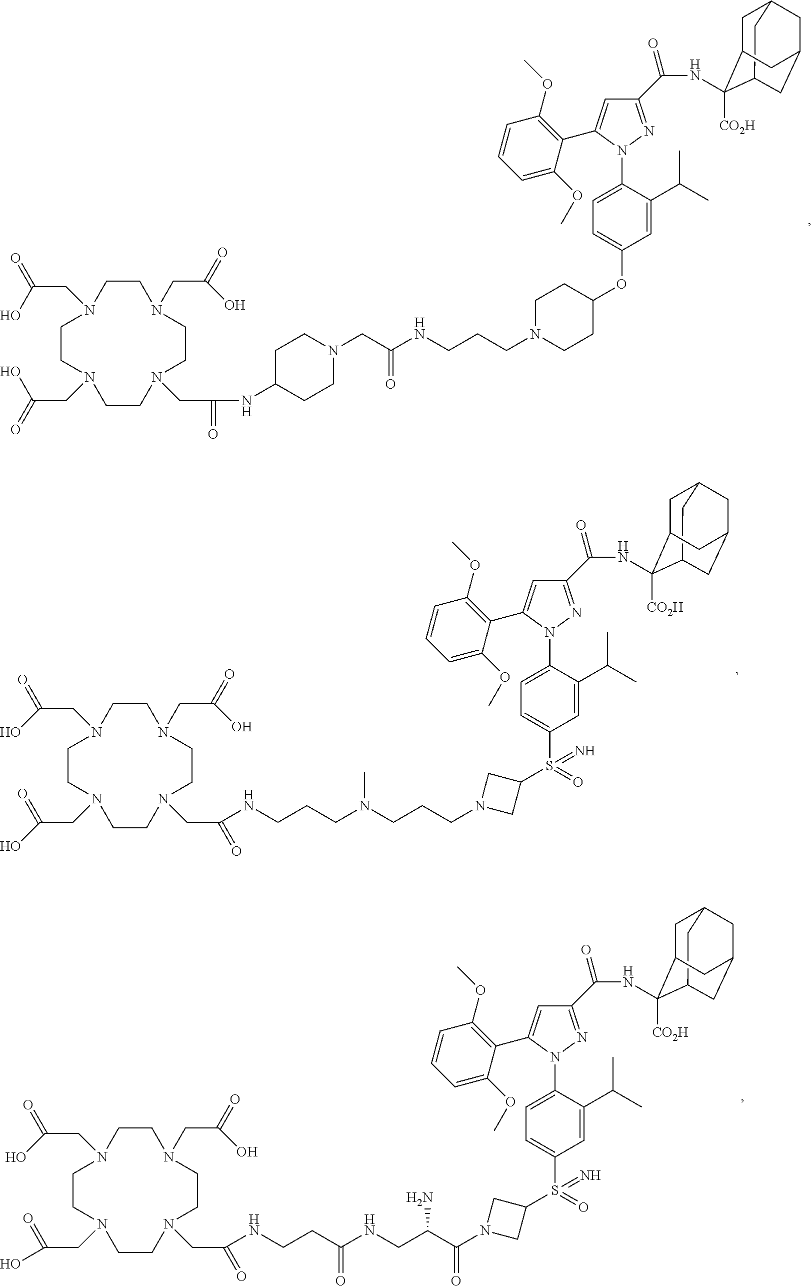 [SMILES: COc1cccc(OC)c1-c1cc(C(=O)NC2(C(=O)O)C3CC4CC(C3)CC2C4)nn1-c1ccc(OC2CCN(CCCNC(=O)CN3CCC(NC(=O)CN4CCN(CC(=O)O)CCN(CC(=O)O)CCN(CC(=O)O)CC4)CC3)CC2)cc1C(C)C ; COc1cccc(OC)c1-c1cc(C(=O)NC2(C(=O)O)C3CC4CC(C3)CC2C4)nn1-c1ccc(S(=N)(=O)C2CN(CCCN(C)CCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(S(=N)(=O)C2CN(C(=O)[C@@H](N)CNC(=O)CCNC(=O)CN3CCN(CC(=O)O)CCN(CC(=O)O)CCN(CC(=O)O)CC3)C2)cc1C(C)C]

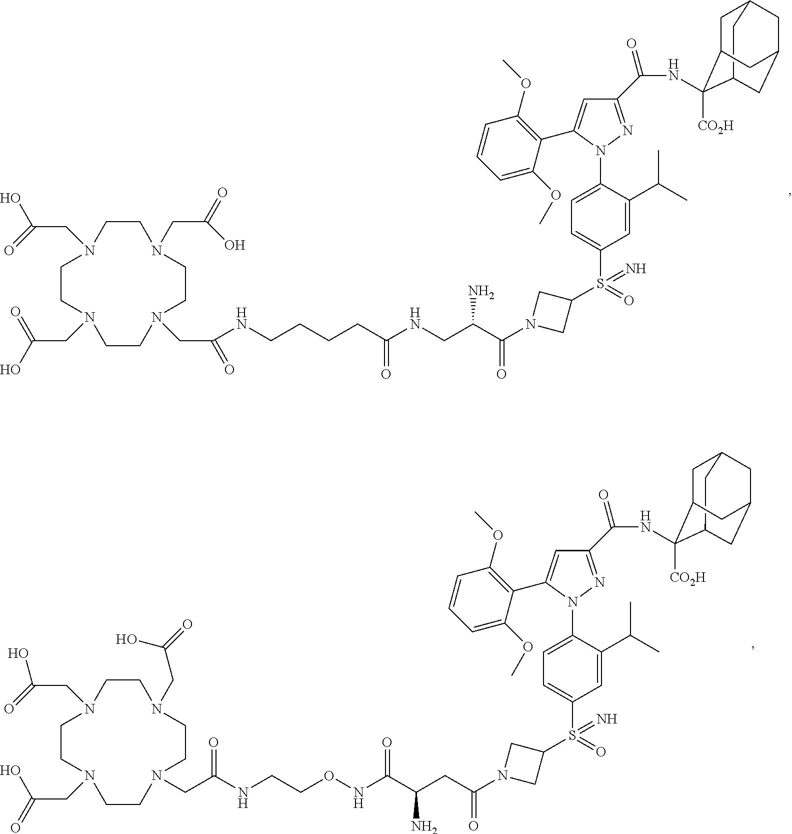 [SMILES: COc1cccc(OC)c1-c1cc(C(=O)NC2(C(=O)O)C3CC4CC(C3)CC2C4)nn1-c1ccc(S(=N)(=O)C2CN(C(=O)[C@@H](N)CNC(=O)CCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(S(=N)(=O)C2CN(C(=O)C[C@@H](N)C(=O)NOCCNC(=O)CN3CCN(CC(=O)O)CCN(CC(=O)O)CCN(CC(=O)O)CC3)C2)cc1C(C)C]

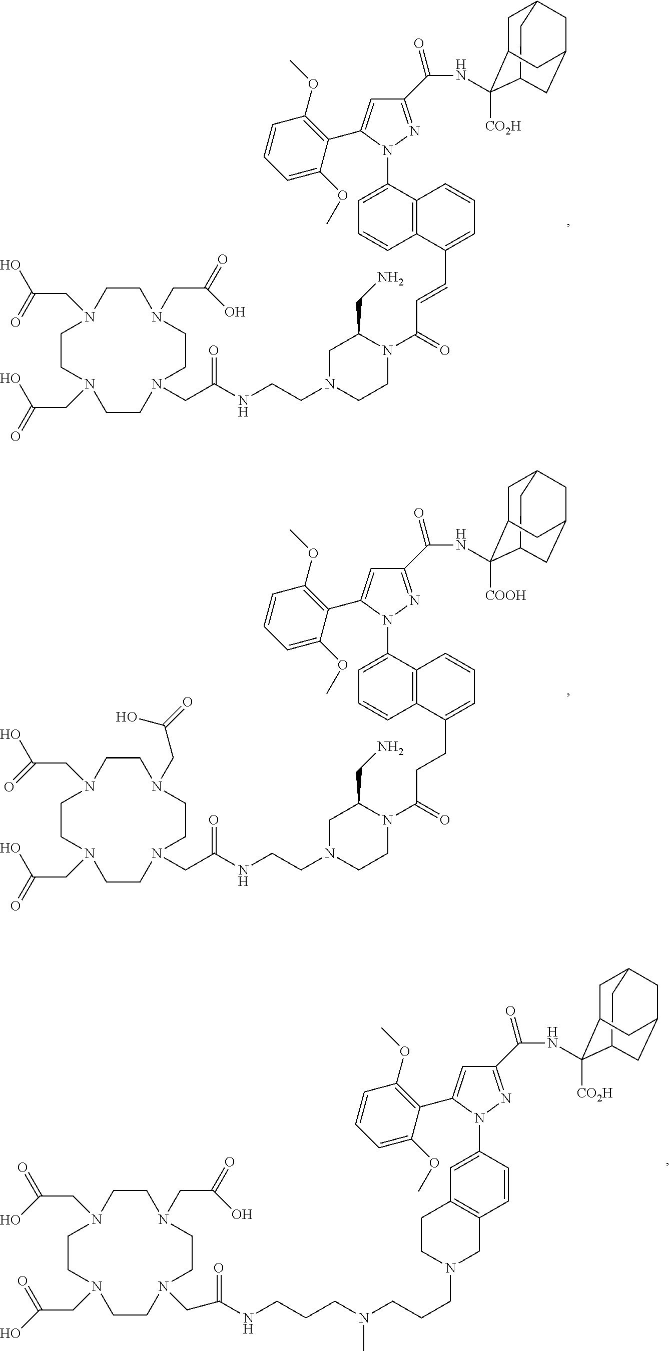 [SMILES: COc1cccc(OC)c1-c1cc(C(=O)NC2(C(=O)O)C3CC4CC(C3)CC2C4)nn1-c1cccc2c(/C=C/C(=O)N3CCN(CCNC(=O)CN4CCN(CC(=O)O)CCN(CC(=O)O)CCN(CC(=O)O)CC4)C[C@H]3CN)cccc12 ; COc1cccc(OC)c1-c1cc(C(=O)NC2(C(=O)O)C3CC4CC(C3)CC2C4)nn1-c1cccc2c(CCC(=O)N3CCN(CCNC(=O)CN4CCN(CC(=O)O)CCN(CC(=O)O)CCN(CC(=O)O)CC4)C[C@H]3CN)cccc12 ; COc1cccc(OC)c1-c1cc(C(=O)NC2(C(=O)O)C3CC4CC(C3)CC2C4)nn1-c1ccc2c(c1)CCN(CCCN(C)CCCNC(=O)CN1CCN(CC(=O)O)CCN(CC(=O)O)CCN(CC(=O)O)CC1)C2]

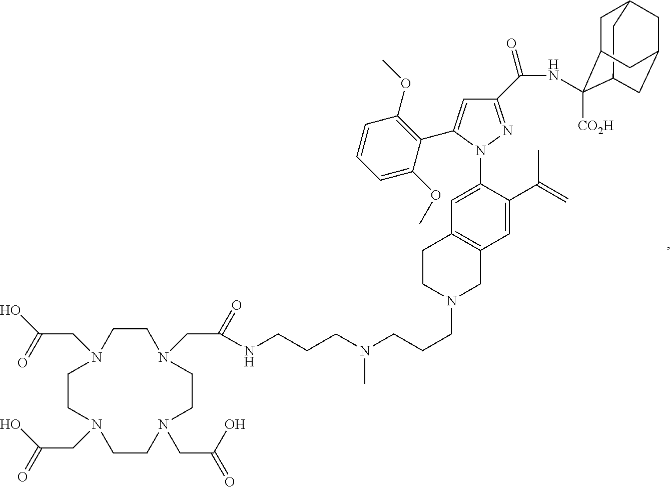 [SMILES: C=C(C)c1cc2c(cc1-n1nc(C(=O)NC3(C(=O)O)C4CC5CC(C4)CC3C5)cc1-c1c(OC)cccc1OC)CCN(CCCN(C)CCCNC(=O)CN1CCN(CC(=O)O)CCN(CC(=O)O)CCN(CC(=O)O)CC1)C2]

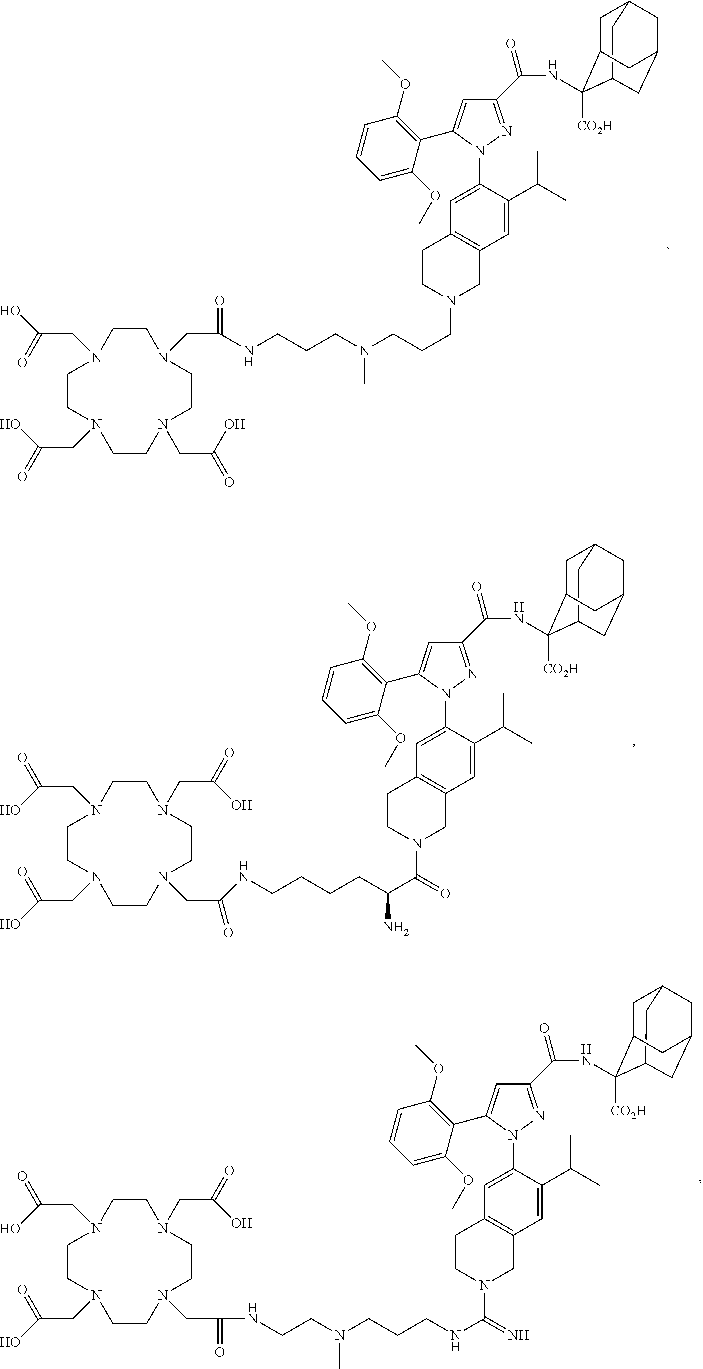 [SMILES: COc1cccc(OC)c1-c1cc(C(=O)NC2(C(=O)O)C3CC4CC(C3)CC2C4)nn1-c1cc2c(cc1C(C)C)CN(CCCN(C)CCCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(C(=O)[C@@H](N)CCCCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(C(=N)NCCCN(C)CCNC(=O)CN1CCN(CC(=O)O)CCN(CC(=O)O)CCN(CC(=O)O)CC1)CC2]

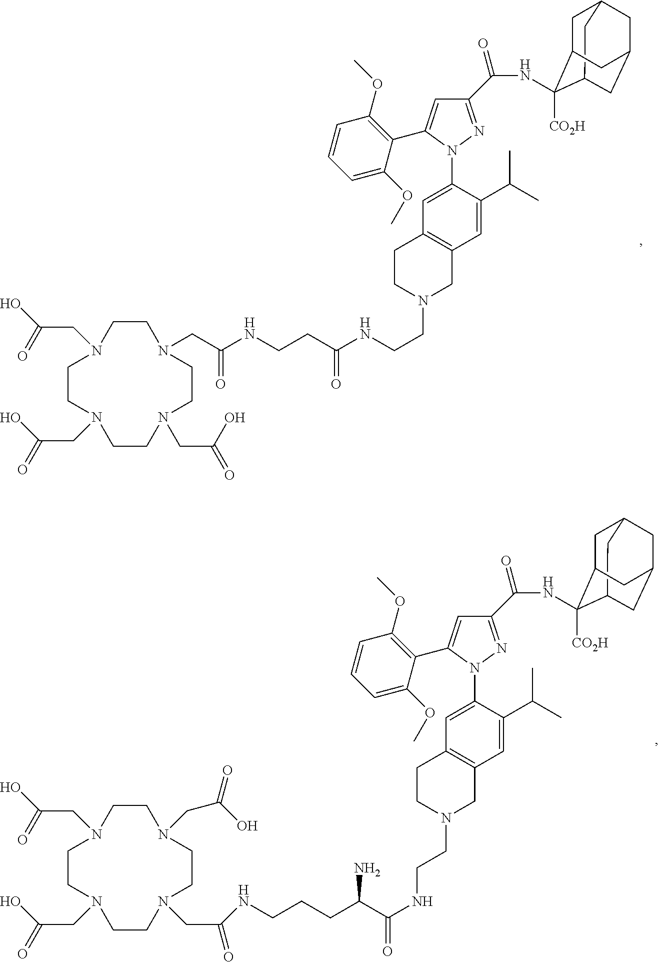 [SMILES: COc1cccc(OC)c1-c1cc(C(=O)NC2(C(=O)O)C3CC4CC(C3)CC2C4)nn1-c1cc2c(cc1C(C)C)CN(CCNC(=O)CCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(CCNC(=O)[C@H](N)CCCNC(=O)CN1CCN(CC(=O)O)CCN(CC(=O)O)CCN(CC(=O)O)CC1)CC2]

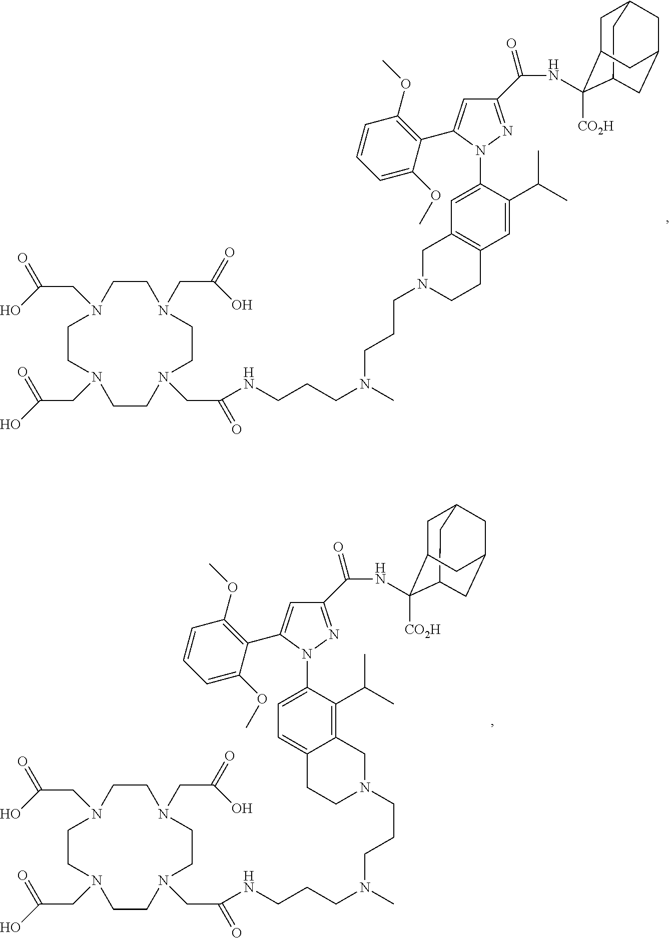 [SMILES: COc1cccc(OC)c1-c1cc(C(=O)NC2(C(=O)O)C3CC4CC(C3)CC2C4)nn1-c1cc2c(cc1C(C)C)CCN(CCCN(C)CCCNC(=O)CN1CCN(CC(=O)O)CCN(CC(=O)O)CCN(CC(=O)O)CC1)C2 ; COc1cccc(OC)c1-c1cc(C(=O)NC2(C(=O)O)C3CC4CC(C3)CC2C4)nn1-c1ccc2c(c1C(C)C)CN(CCCN(C)CCCNC(=O)CN1CCN(CC(=O)O)CCN(CC(=O)O)CCN(CC(=O)O)CC1)CC2]

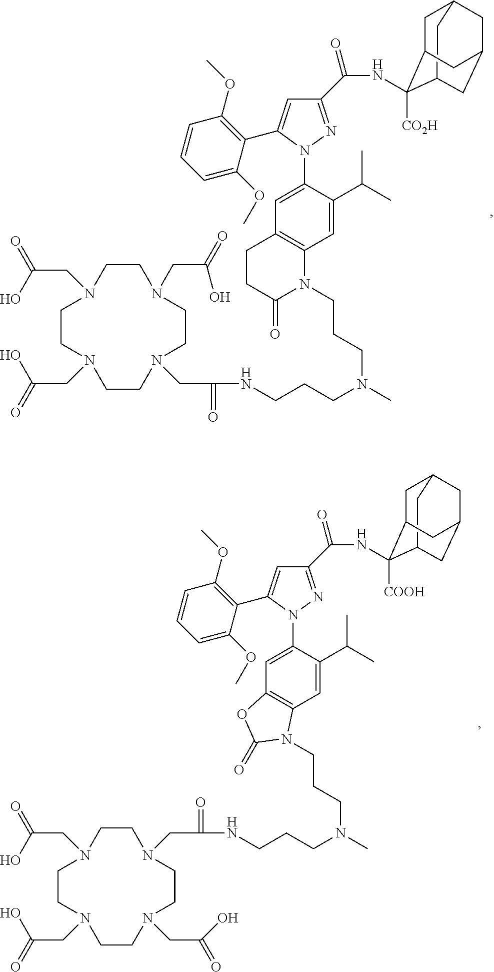 [SMILES: COc1cccc(OC)c1-c1cc(C(=O)NC2(C(=O)O)C3CC4CC(C3)CC2C4)nn1-c1cc2c(cc1C(C)C)N(CCCN(C)CCCNC(=O)CN1CCN(CC(=O)O)CCN(CC(=O)O)CCN(CC(=O)O)CC1)C(=O)CC2 ; COc1cccc(OC)c1-c1cc(C(=O)NC2(C(=O)O)C3CC4CC(C3)CC2C4)nn1-c1cc2oc(=O)n(CCCN(C)CCCNC(=O)CN3CCN(CC(=O)O)CCN(CC(=O)O)CCN(CC(=O)O)CC3)c2cc1C(C)C]

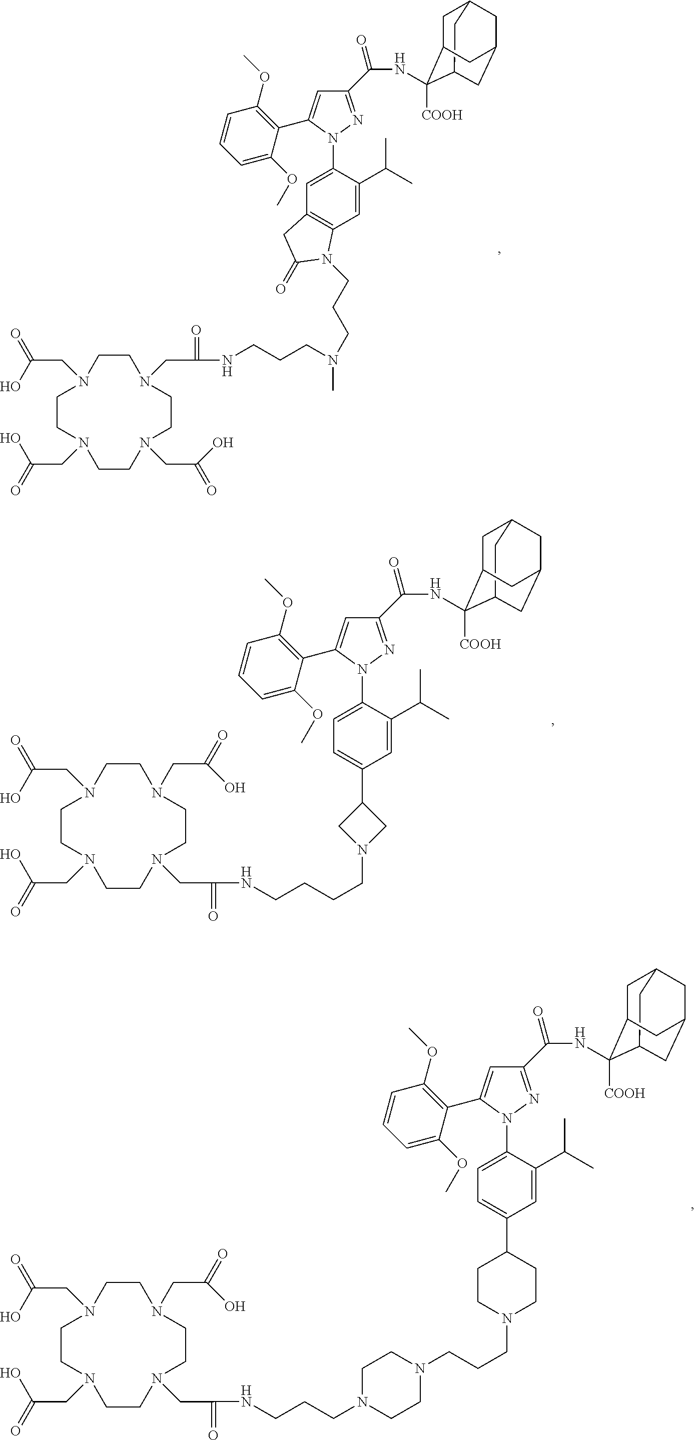 [SMILES: COc1cccc(OC)c1-c1cc(C(=O)NC2(C(=O)O)C3CC4CC(C3)CC2C4)nn1-c1cc2c(cc1C(C)C)N(CCCN(C)CCCNC(=O)CN1CCN(CC(=O)O)CCN(CC(=O)O)CCN(CC(=O)O)CC1)C(=O)C2 ; COc1cccc(OC)c1-c1cc(C(=O)NC2(C(=O)O)C3CC4CC(C3)CC2C4)nn1-c1ccc(C2CN(CCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCN3CCN(CCCNC(=O)CN4CCN(CC(=O)O)CCN(CC(=O)O)CCN(CC(=O)O)CC4)CC3)CC2)cc1C(C)C]

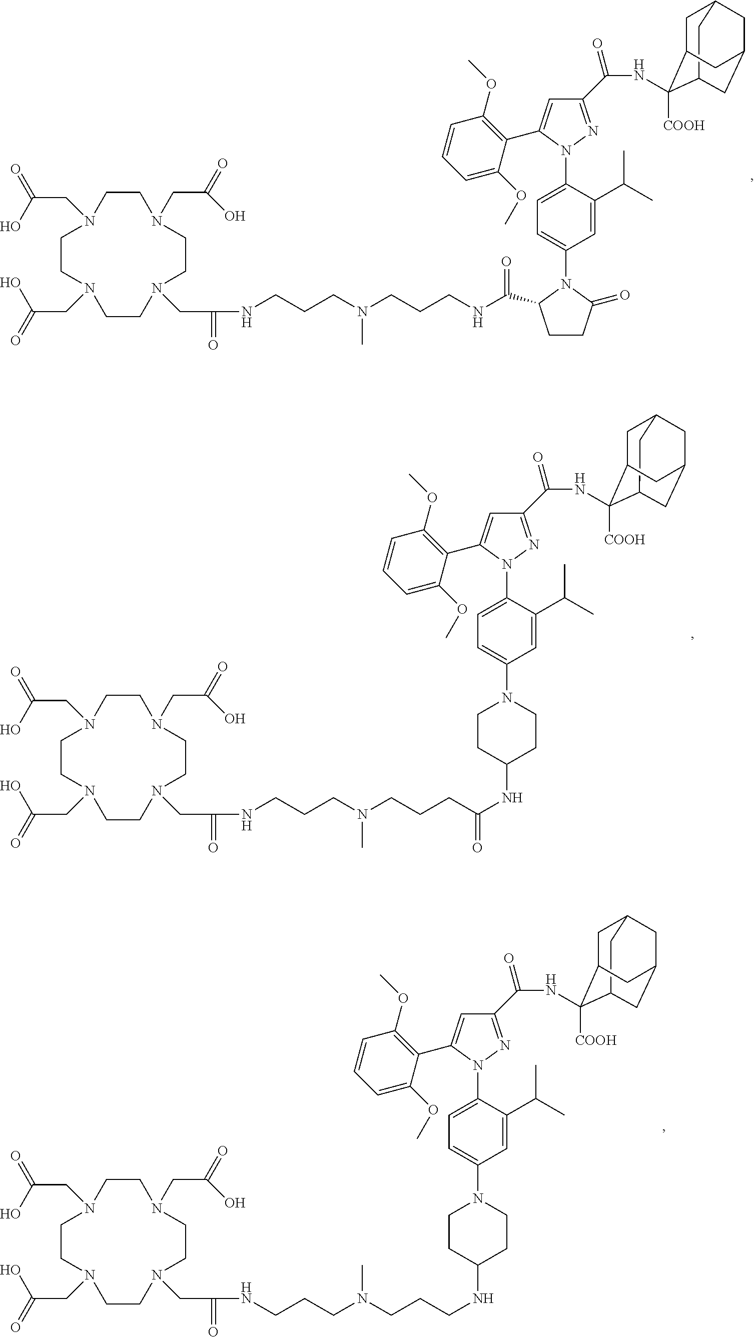 [SMILES: COc1cccc(OC)c1-c1cc(C(=O)NC2(C(=O)O)C3CC4CC(C3)CC2C4)nn1-c1ccc(N2C(=O)CC[C@@H]2C(=O)NCCCN(C)CCCNC(=O)CN2CCN(CC(=O)O)CCN(CC(=O)O)CCN(CC(=O)O)CC2)cc1C(C)C ; COc1cccc(OC)c1-c1cc(C(=O)NC2(C(=O)O)C3CC4CC(C3)CC2C4)nn1-c1ccc(N2CCC(NC(=O)CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCC(NCCCN(C)CCCNC(=O)CN3CCN(CC(=O)O)CCN(CC(=O)O)CCN(CC(=O)O)CC3)CC2)cc1C(C)C]

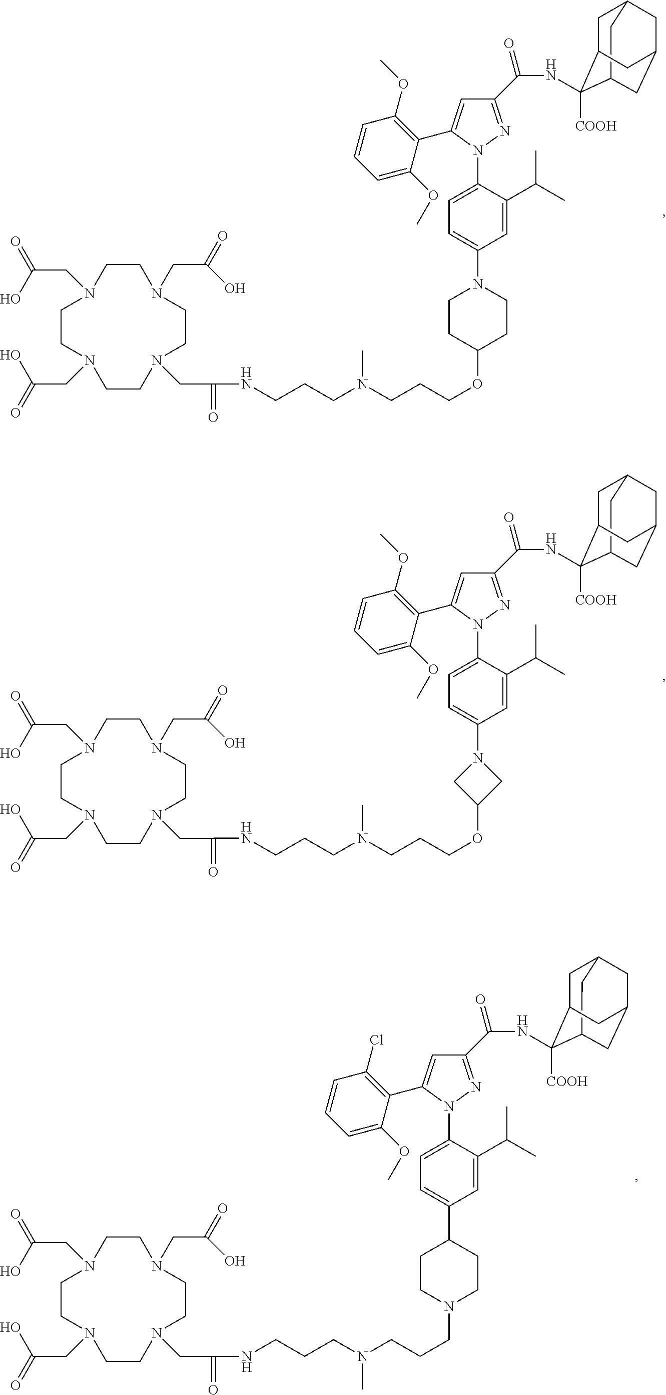 [SMILES: COc1cccc(OC)c1-c1cc(C(=O)NC2(C(=O)O)C3CC4CC(C3)CC2C4)nn1-c1ccc(N2CCC(OCCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CC(OCCCN(C)CCCNC(=O)CN3CCN(CC(=O)O)CCN(CC(=O)O)CCN(CC(=O)O)CC3)C2)cc1C(C)C ; COc1cccc(Cl)c1-c1cc(C(=O)NC2(C(=O)O)C3CC4CC(C3)CC2C4)nn1-c1ccc(C2CCN(CCCN(C)CCCNC(=O)CN3CCN(CC(=O)O)CCN(CC(=O)O)CCN(CC(=O)O)CC3)CC2)cc1C(C)C]

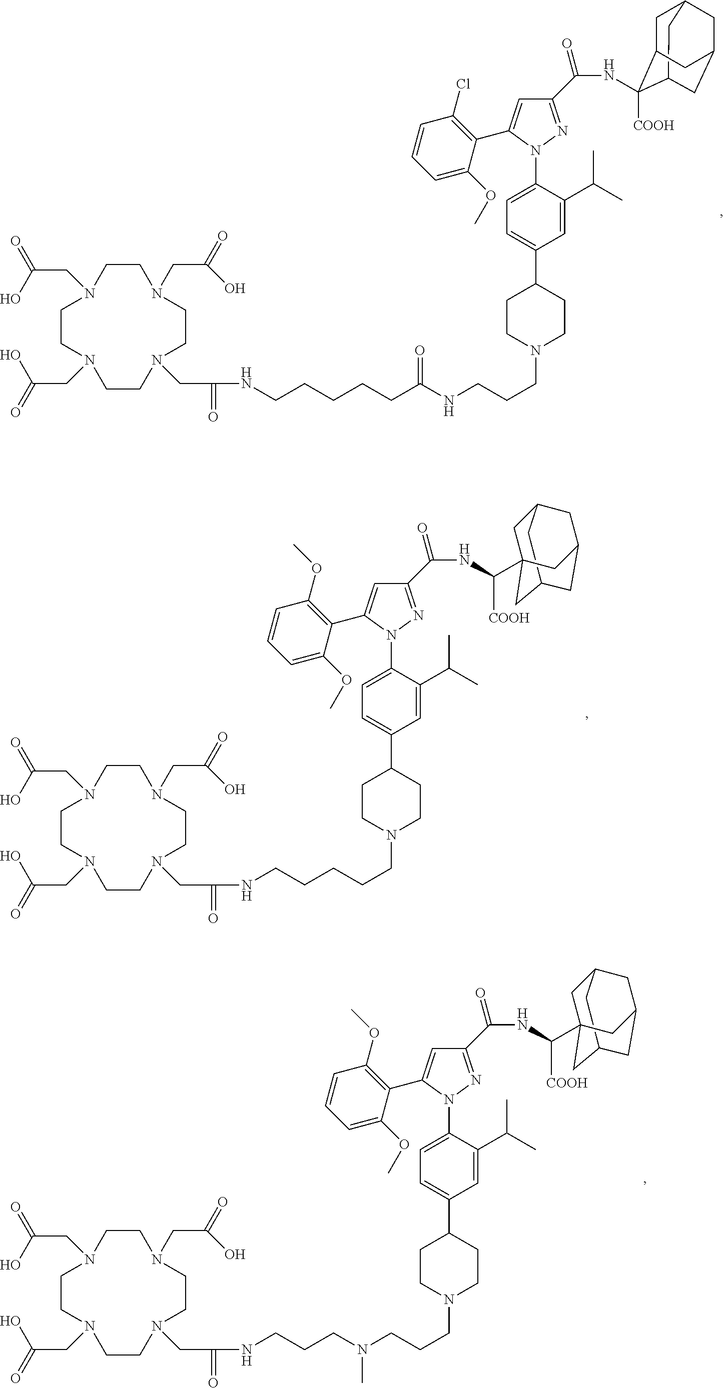 [SMILES: COc1cccc(Cl)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2CCN(CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2CCN(CCCN(C)CCCNC(=O)CN3CCN(CC(=O)O)CCN(CC(=O)O)CCN(CC(=O)O)CC3)CC2)cc1C(C)C]

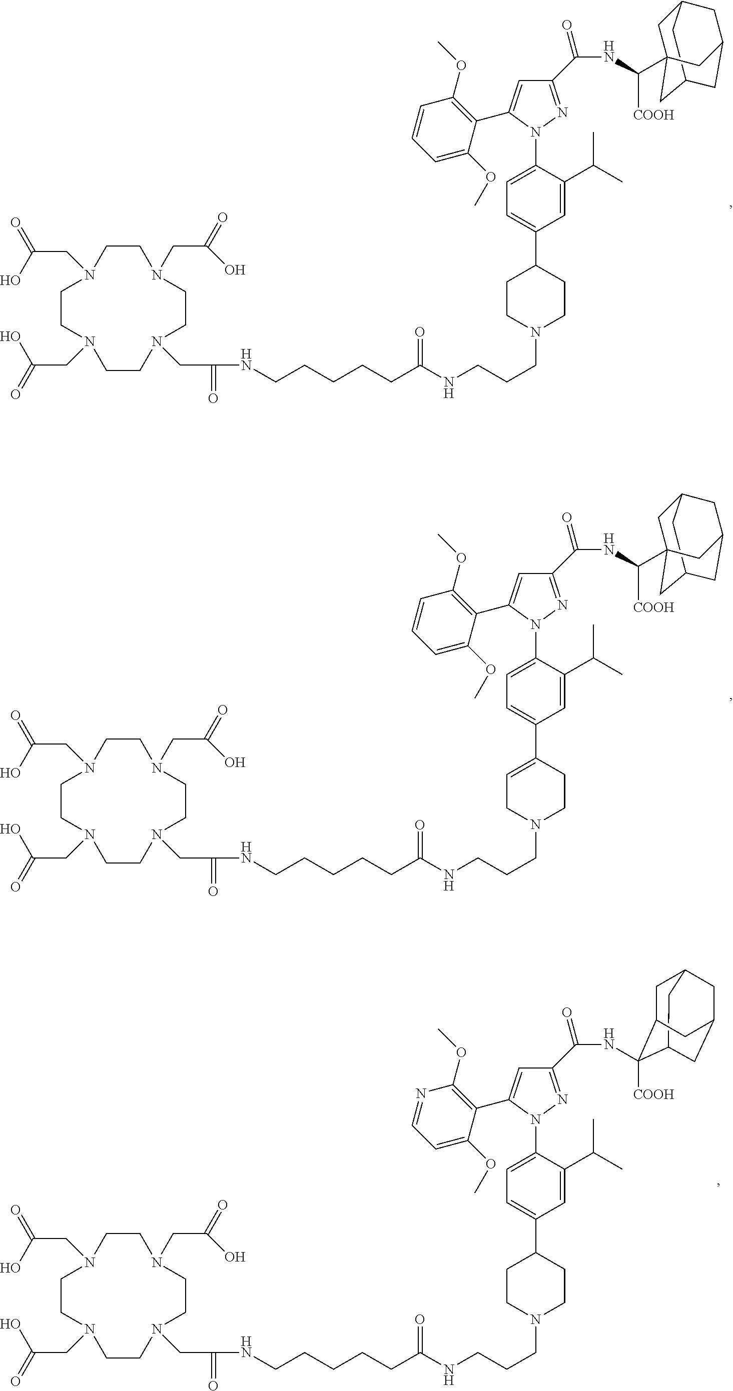 [SMILES: COc1cccc(OC)c1-c1cc(C(=O)N[C@H](C(=O)O)C23CC4CC(CC(C4)C2)C3)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2=CCN(CCCNC(=O)CCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C]

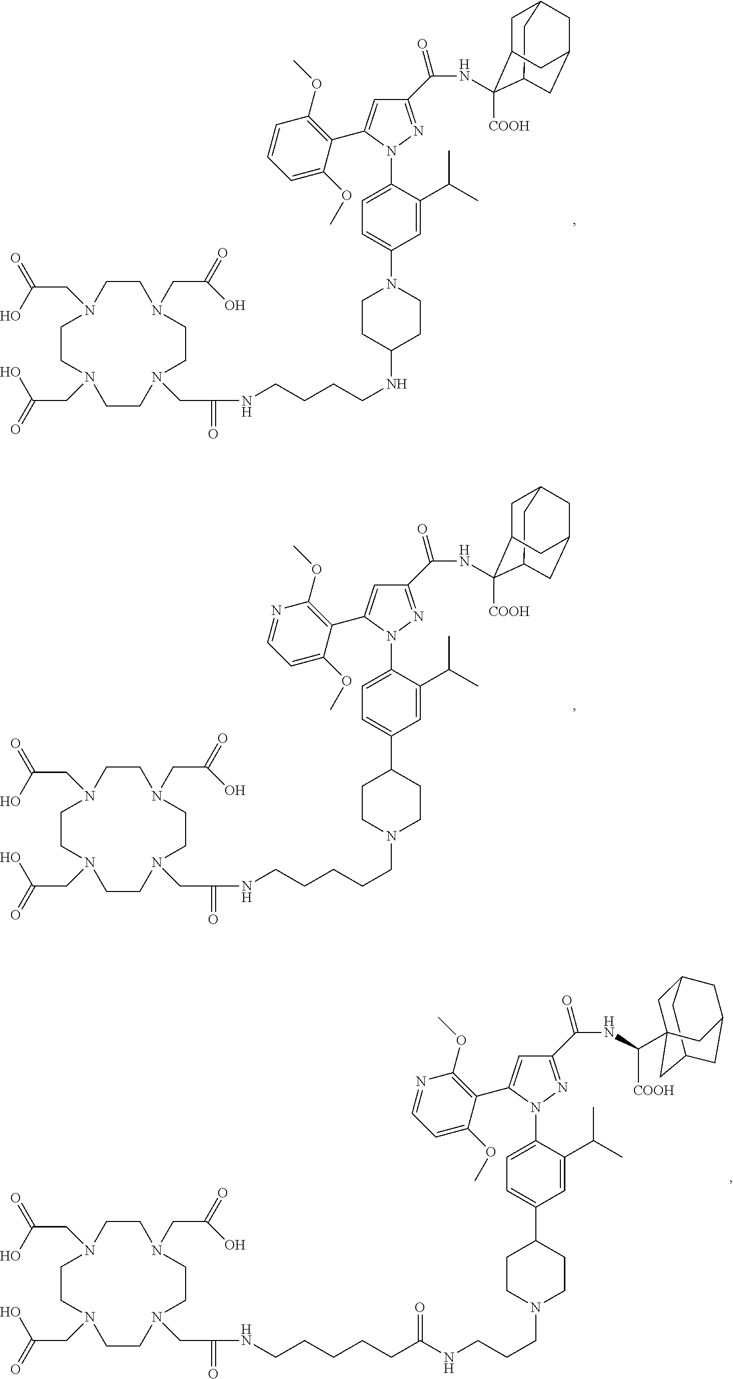 [SMILES: COc1cccc(OC)c1-c1cc(C(=O)NC2(C(=O)O)C3CC4CC(C3)CC2C4)nn1-c1ccc(N2CCC(NCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)N[C@H](C(=O)O)C23CC4CC(CC(C4)C2)C3)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C]

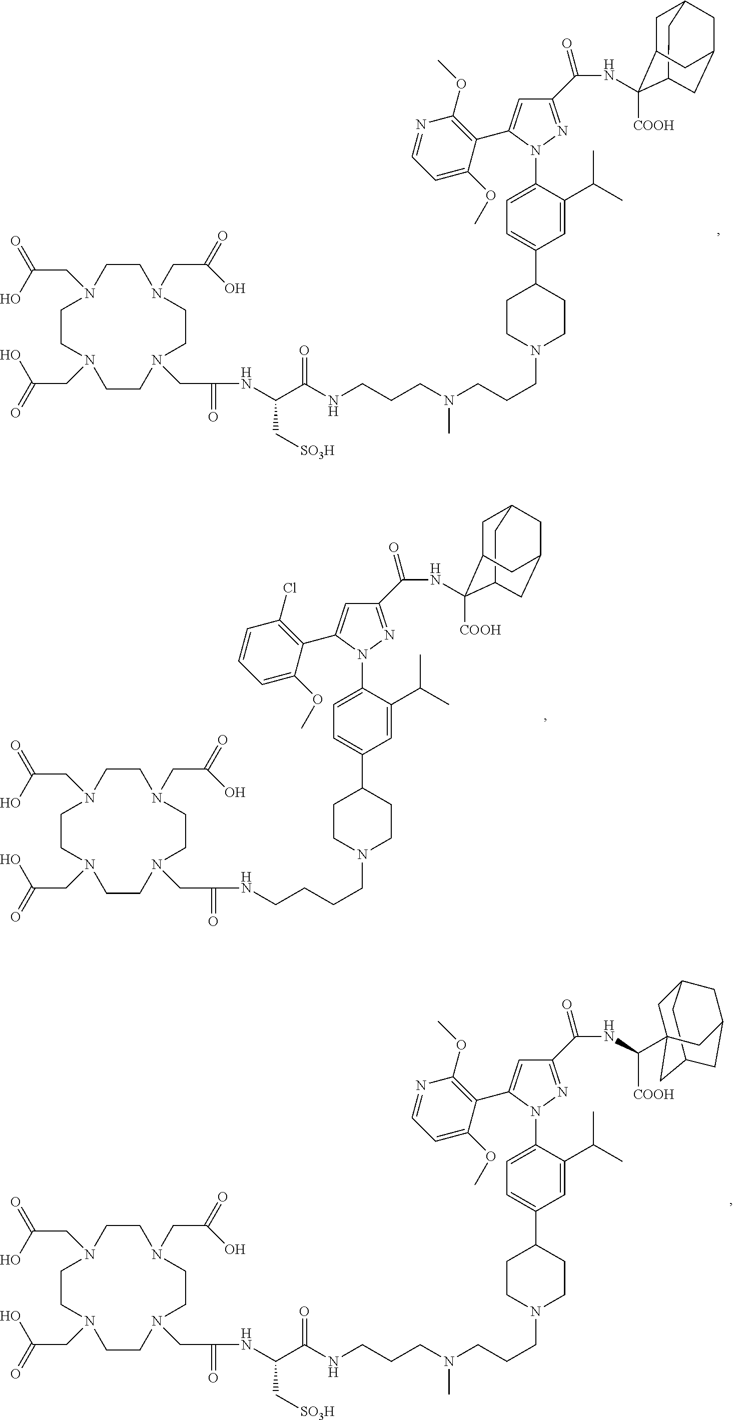 [SMILES: COc1ccnc(OC)c1-c1cc(C(=O)NC2(C(=O)O)C3CC4CC(C3)CC2C4)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C ; COc1cccc(Cl)c1-c1cc(C(=O)NC2(C(=O)O)C3CC4CC(C3)CC2C4)nn1-c1ccc(C2CCN(CCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)N[C@H](C(=O)O)C23CC4CC(CC(C4)C2)C3)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C]

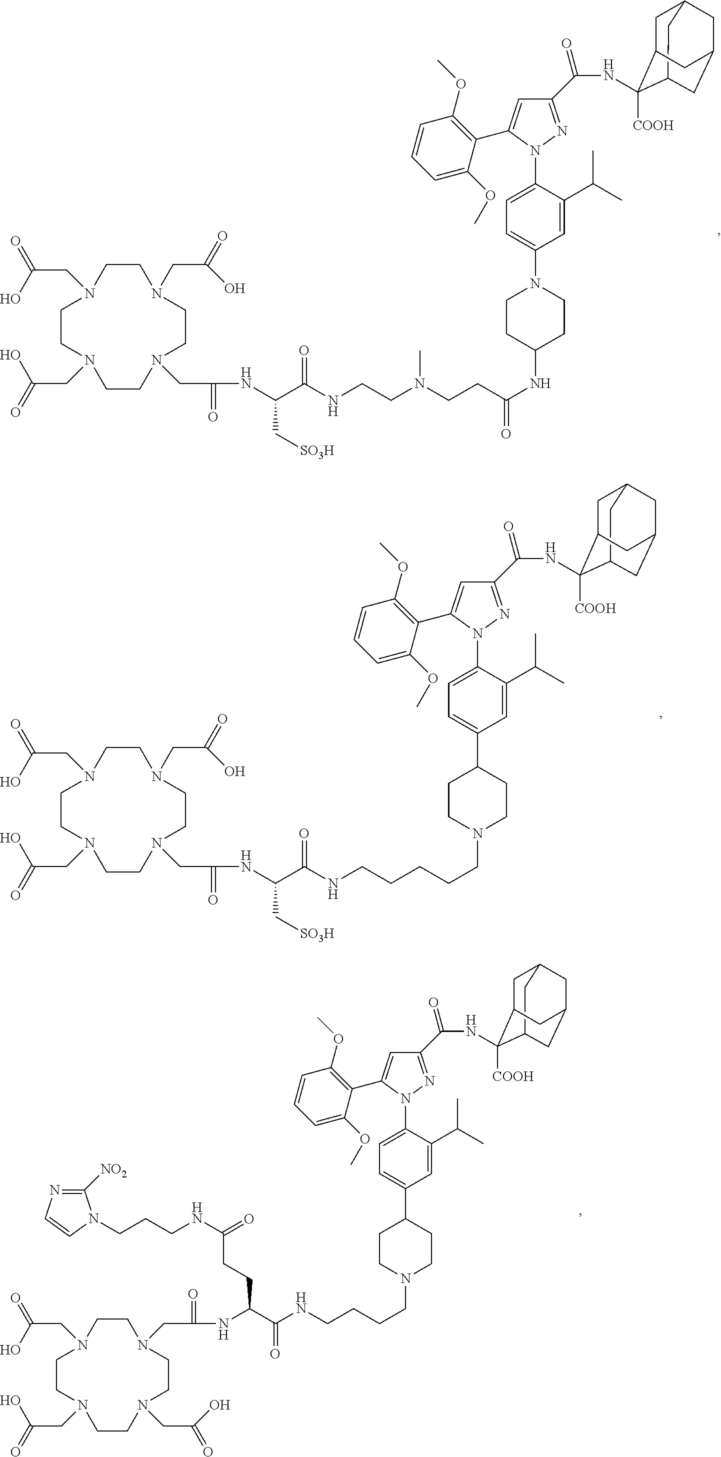

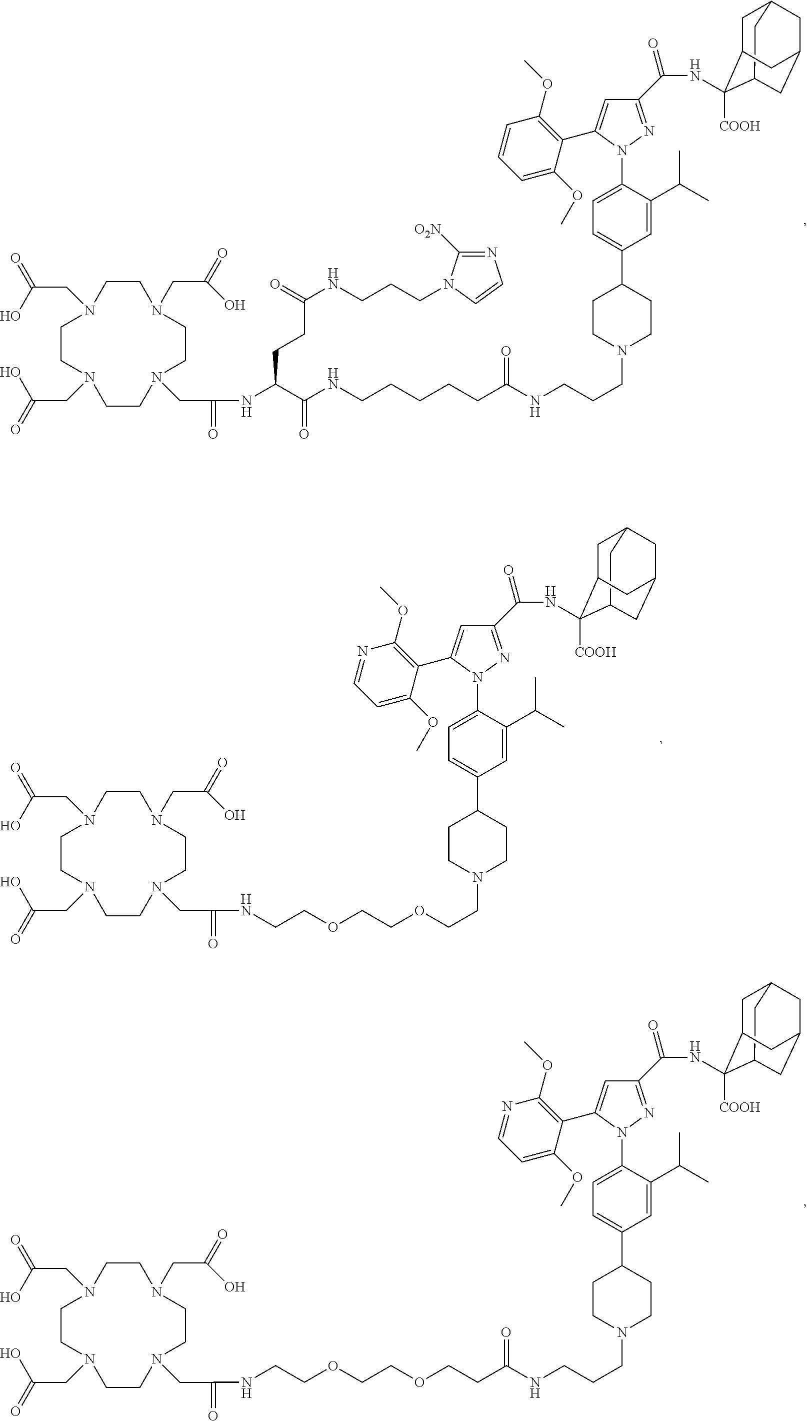 [SMILES: COc1cccc(OC)c1-c1cc(C(=O)NC2(C(=O)O)C3CC4CC(C3)CC2C4)nn1-c1ccc(C2CCN(CCCNC(=O)CCCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCOCCOCCNC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCNC(=O)CCOCCOCCNC(=O)CN3CCN(CC(=O)O)CCN(CC(=O)O)CCN(CC(=O)O)CC3)CC2)cc1C(C)C]

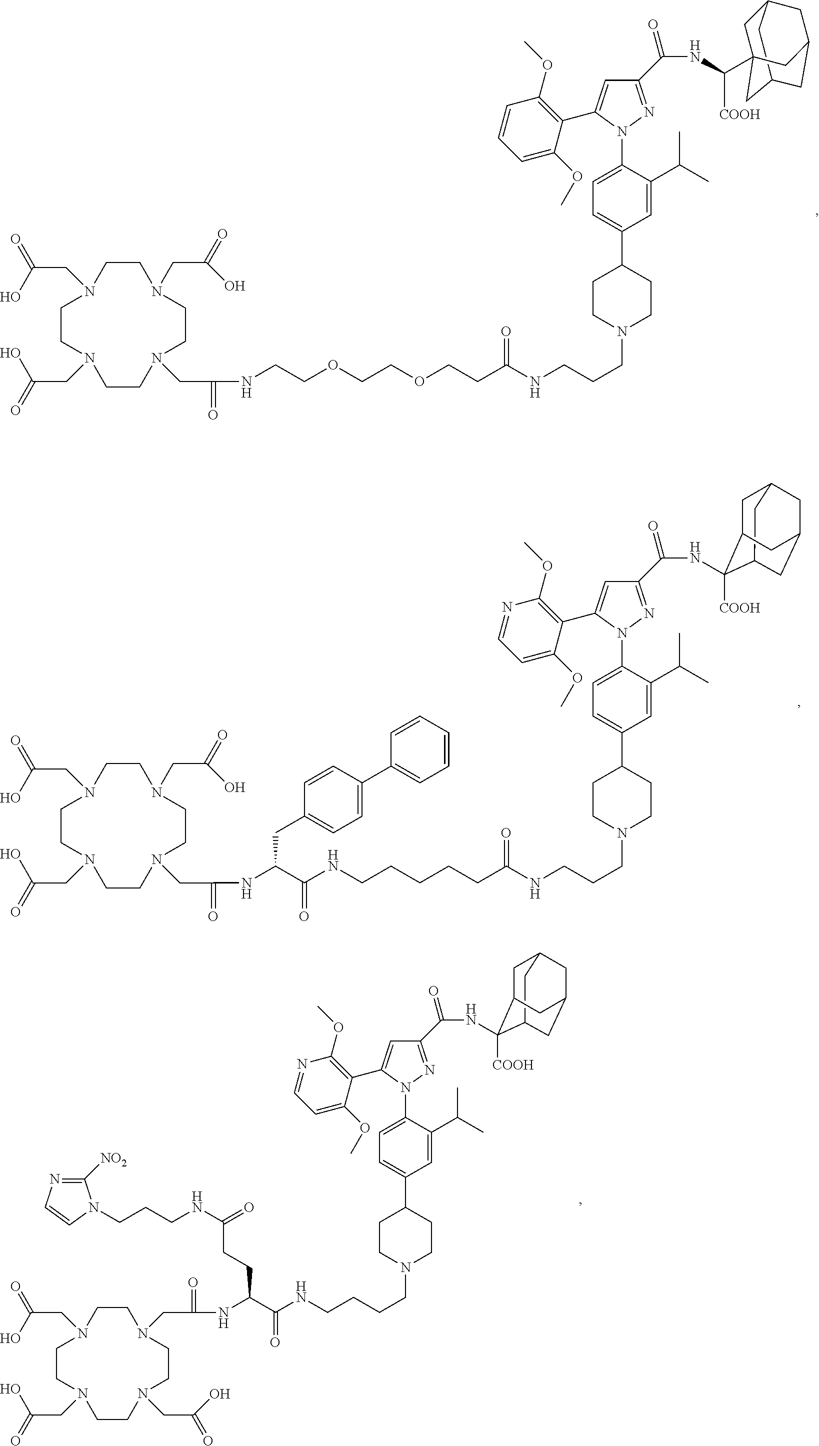

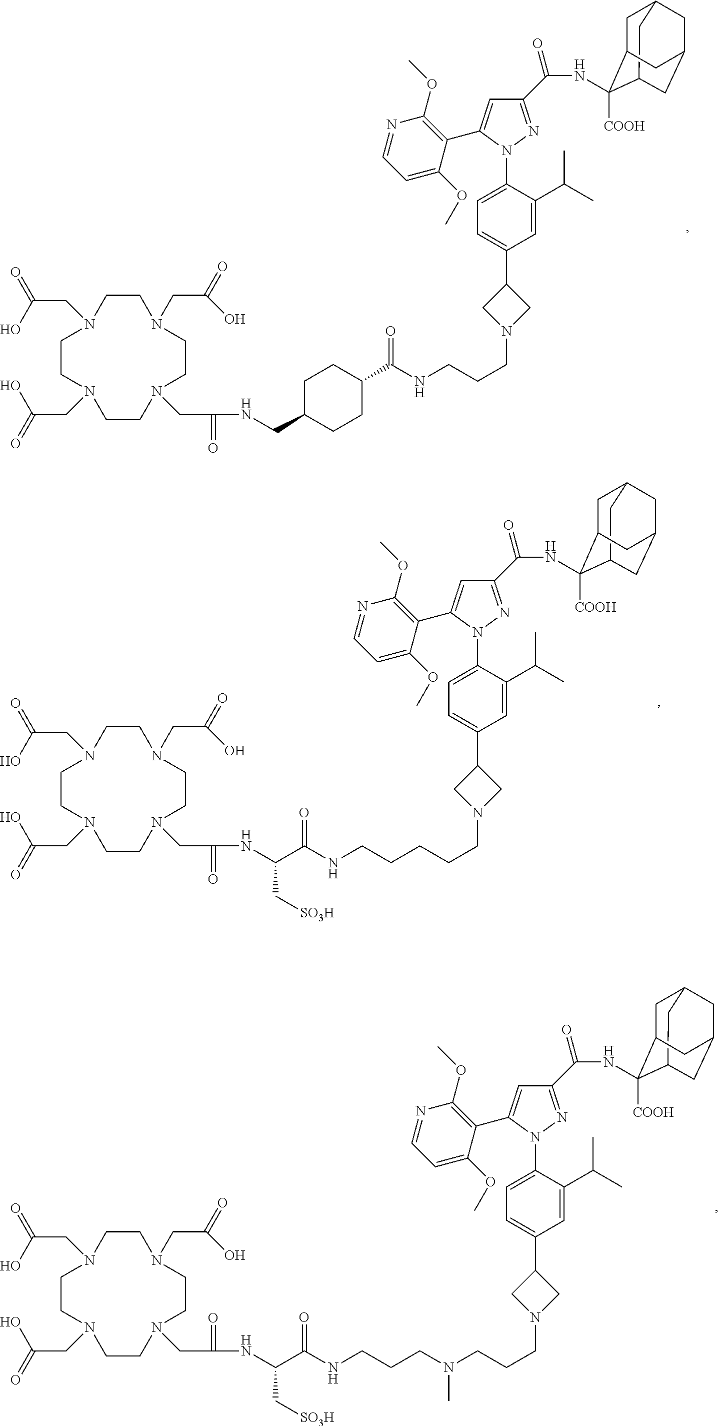 [SMILES: COc1ccnc(OC)c1-c1cc(C(=O)NC2(C(=O)O)C3CC4CC(C3)CC2C4)nn1-c1ccc(C2CN(CCCNC(=O)[C@H]3CC[C@H](CNC(=O)CN4CCN(CC(=O)O)CCN(CC(=O)O)CCN(CC(=O)O)CC4)CC3)C2)cc1C(C)C ; COc1ccnc(OC)c1-c1cc(C(=O)NC2(C(=O)O)C3CC4CC(C3)CC2C4)nn1-c1ccc(C2CN(CCCCCNC(=O)[C@H](CS(=O)(=O)O)NC(=O)CN3CCN(CC(=O)O)CCN(CC(=O)O)CCN(CC(=O)O)CC3)C2)cc1C(C)C ; COc1ccnc(OC)c1-c1cc(C(=O)NC2(C(=O)O)C3CC4CC(C3)CC2C4)nn1-c1ccc(C2CN(CCCN(C)CCCNC(=O)[C@H](CS(=O)(=O)O)NC(=O)CN3CCN(CC(=O)O)CCN(CC(=O)O)CCN(CC(=O)O)CC3)C2)cc1C(C)C]

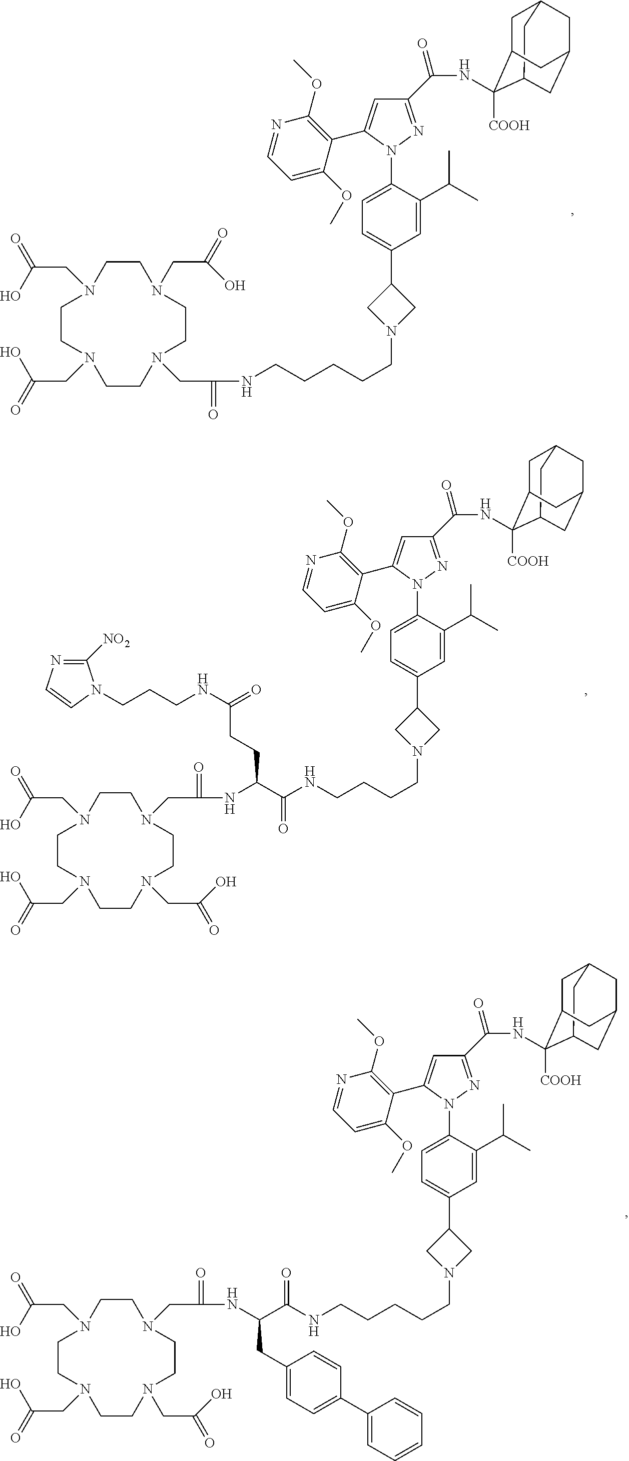 [SMILES: COc1ccnc(OC)c1-c1cc(C(=O)NC2(C(=O)O)C3CC4CC(C3)CC2C4)nn1-c1ccc(C2CN(CCCCCNC(=O)CN3CCN(CC(=O)O)CCN(CC(=O)O)CCN(CC(=O)O)CC3)C2)cc1C(C)C ; COc1ccnc(OC)c1-c1cc(C(=O)NC2(C(=O)O)C3CC4CC(C3)CC2C4)nn1-c1ccc(C2CN(CCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)C2)cc1C(C)C ; COc1ccnc(OC)c1-c1cc(C(=O)NC2(C(=O)O)C3CC4CC(C3)CC2C4)nn1-c1ccc(C2CN(CCCCCNC(=O)[C@@H](Cc3ccc(-c4ccccc4)cc3)NC(=O)CN3CCN(CC(=O)O)CCN(CC(=O)O)CCN(CC(=O)O)CC3)C2)cc1C(C)C]

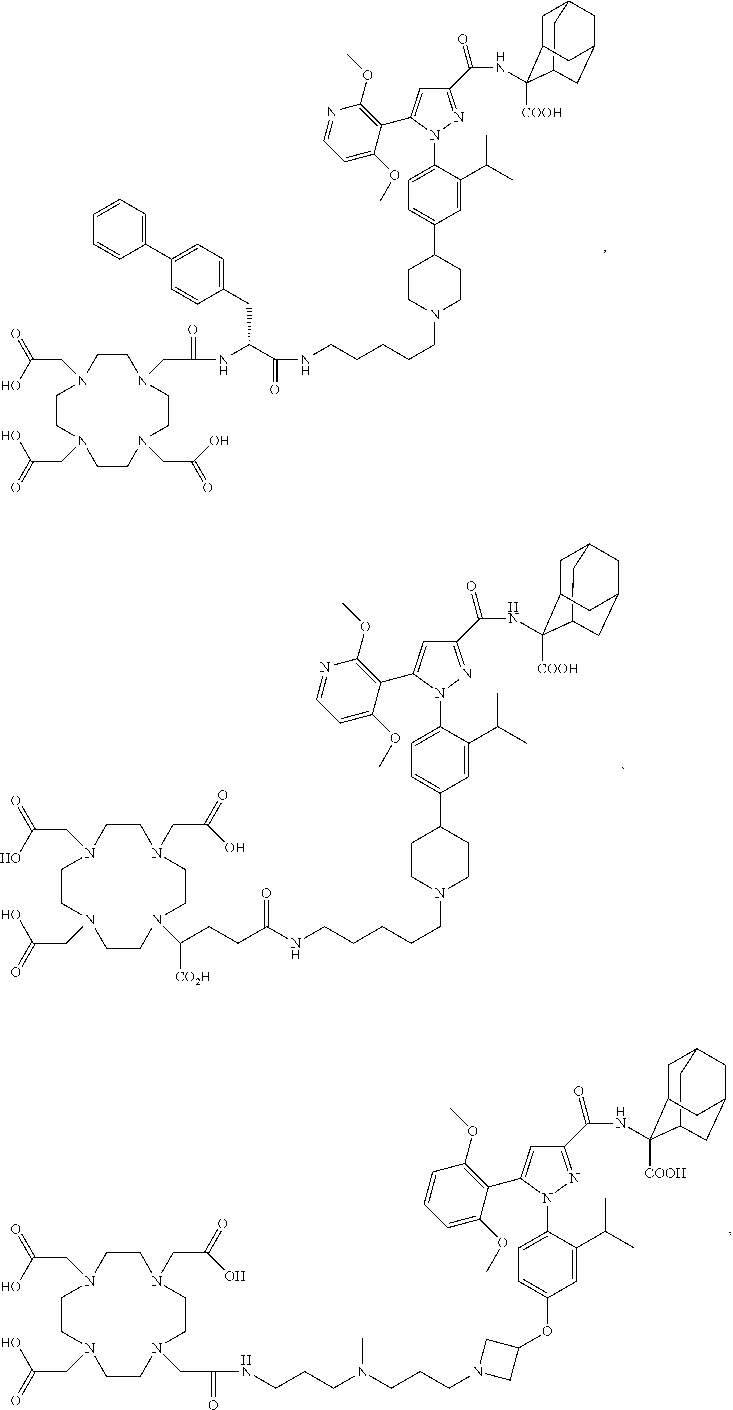 [SMILES: COc1ccnc(OC)c1-c1cc(C(=O)NC2(C(=O)O)C3CC4CC(C3)CC2C4)nn1-c1ccc(C2CCN(CCCCCNC(=O)[C@@H](Cc3ccc(-c4ccccc4)cc3)NC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCCCNC(=O)CCC(C(=O)O)N3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(OC2CN(CCCN(C)CCCNC(=O)CN3CCN(CC(=O)O)CCN(CC(=O)O)CCN(CC(=O)O)CC3)C2)cc1C(C)C]

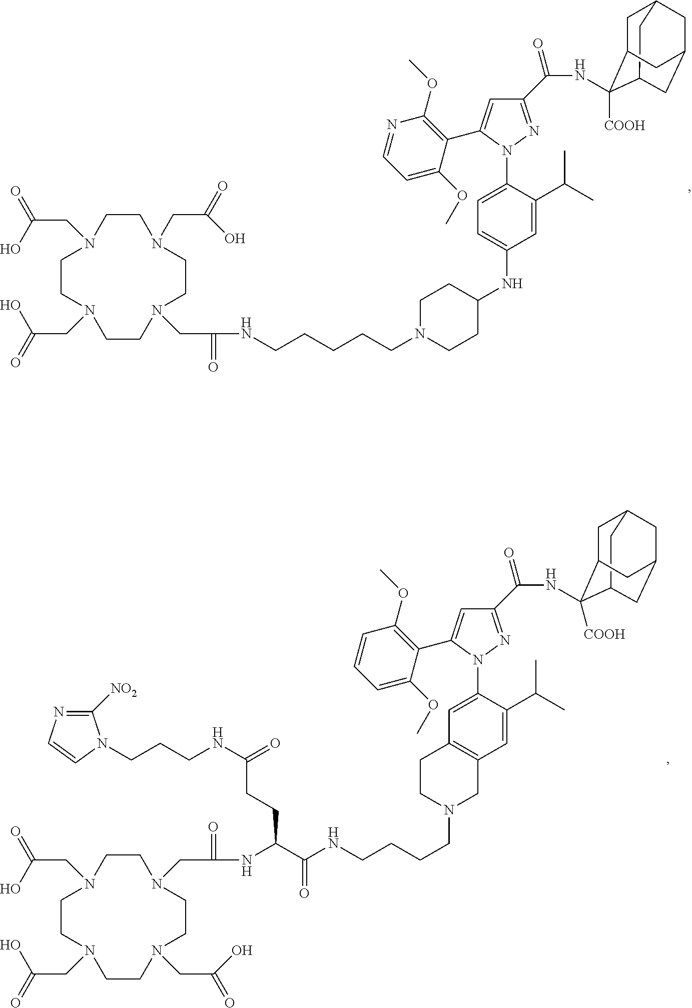 [SMILES: COc1ccnc(OC)c1-c1cc(C(=O)NC2(C(=O)O)C3CC4CC(C3)CC2C4)nn1-c1ccc(NC2CCN(CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1cc2c(cc1C(C)C)CN(CCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1)CC2]

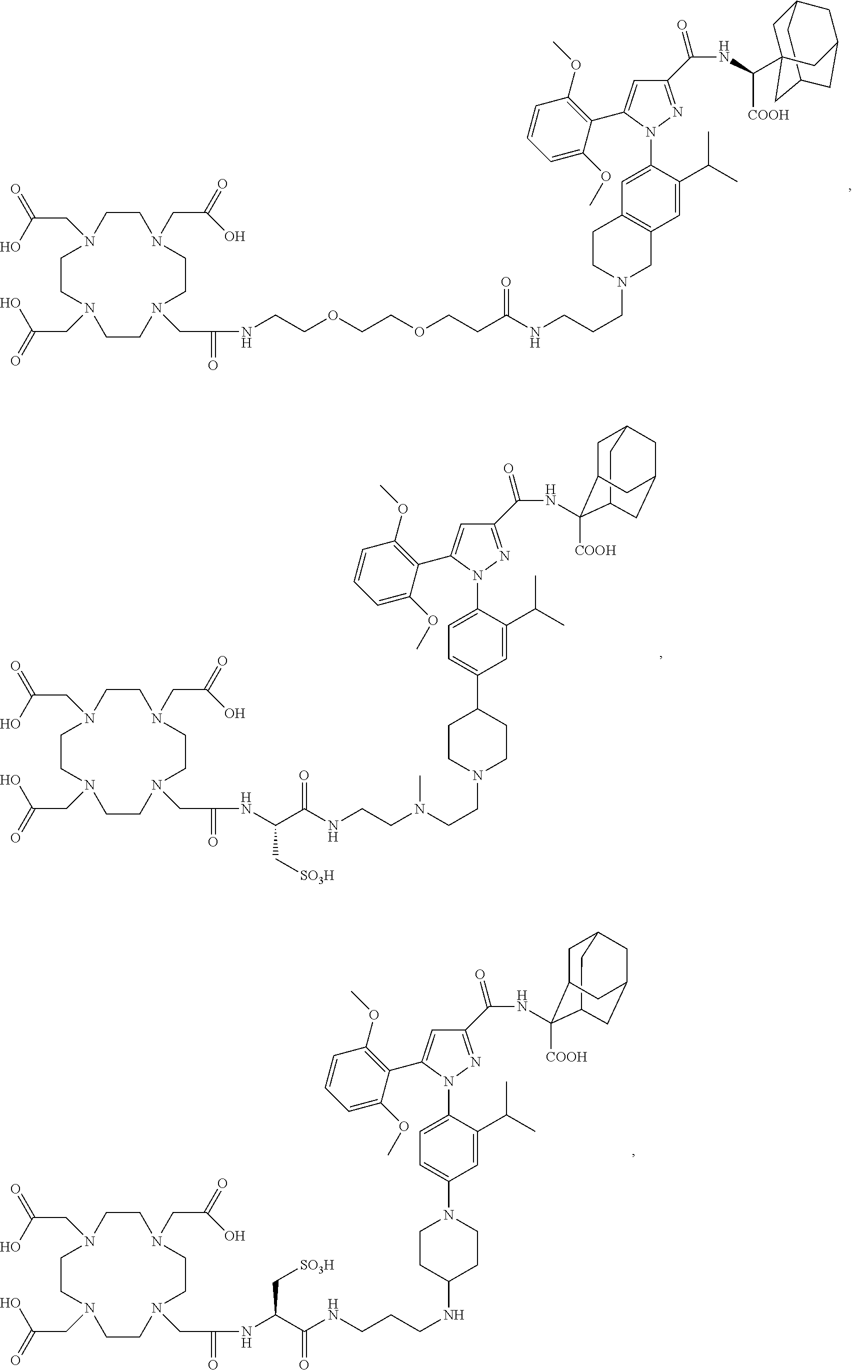 [SMILES: COc1cccc(OC)c1-c1cc(C(=O)N[C@H](C(=O)O)C23CC4CC(CC(C4)C2)C3)nn1-c1cc2c(cc1C(C)C)CN(CCCNC(=O)CCOCCOCCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1ccc(C2CCN(CCN(C)CCNC(=O)[C@H](CS(=O)(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(N2CCC(NCCCNC(=O)[C@H](CS(=O)(=O)O)NC(=O)CN3CCN(CC(=O)O)CCN(CC(=O)O)CCN(CC(=O)O)CC3)CC2)cc1C(C)C]

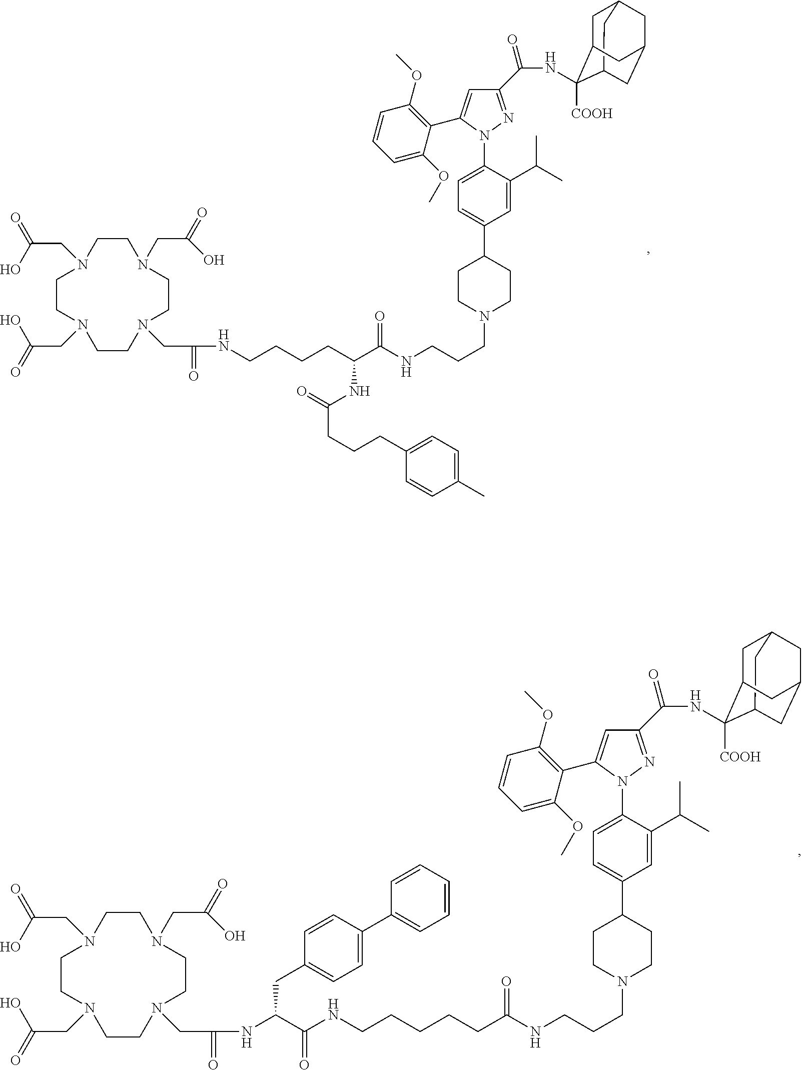 [SMILES: COc1cccc(OC)c1-c1cc(C(=O)NC2(C(=O)O)C3CC4CC(C3)CC2C4)nn1-c1ccc(C2CCN(CCCNC(=O)[C@@H](CCCCNC(=O)CN3CCN(CC(=O)O)CCN(CC(=O)O)CCN(CC(=O)O)CC3)NC(=O)CCCc3ccc(C)cc3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CCCCCNC(=O)[C@@H](Cc3ccc(-c4ccccc4)cc3)NC(=O)CN3CCN(CC(=O)O)CCN(CC(=O)O)CCN(CC(=O)O)CC3)CC2)cc1C(C)C]

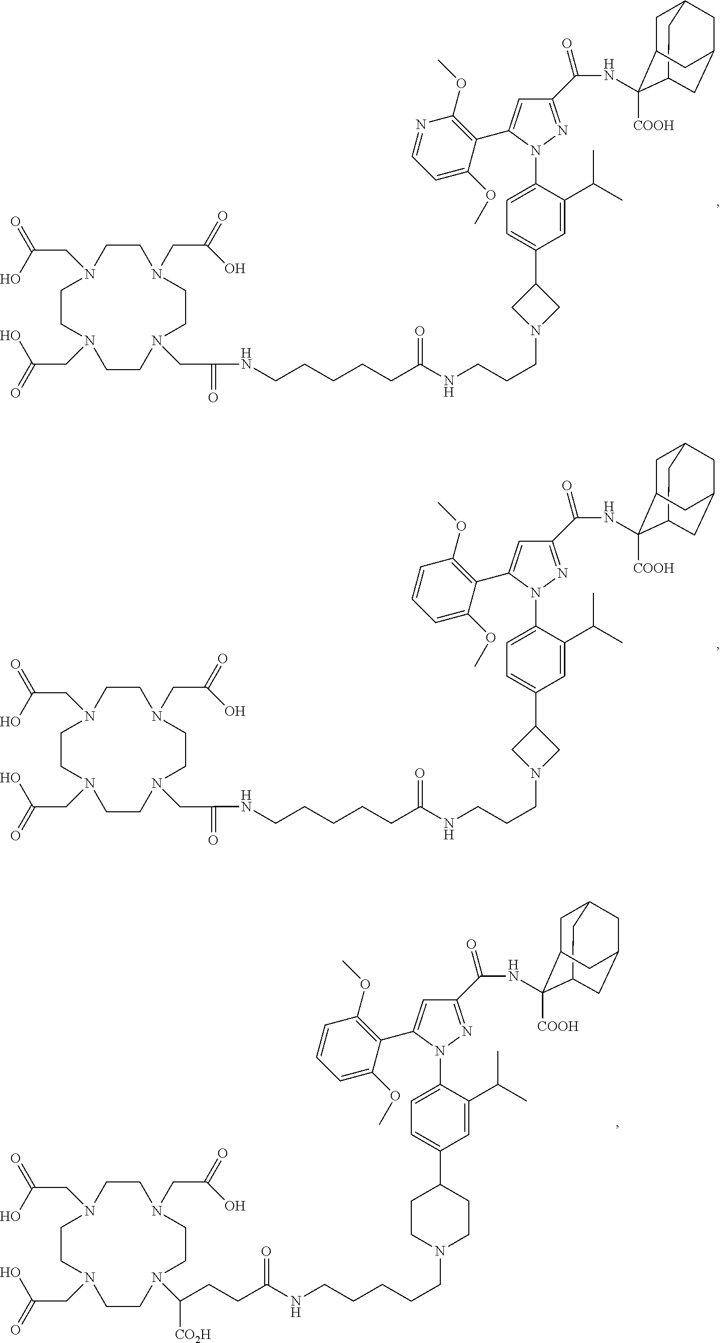 [SMILES: COc1ccnc(OC)c1-c1cc(C(=O)NC2(C(=O)O)C3CC4CC(C3)CC2C4)nn1-c1ccc(C2CN(CCCNC(=O)CCCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CN(CCCNC(=O)CCCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCCCNC(=O)CCC(C(=O)O)N3CCN(CC(=O)O)CCN(CC(=O)O)CCN(CC(=O)O)CC3)CC2)cc1C(C)C]

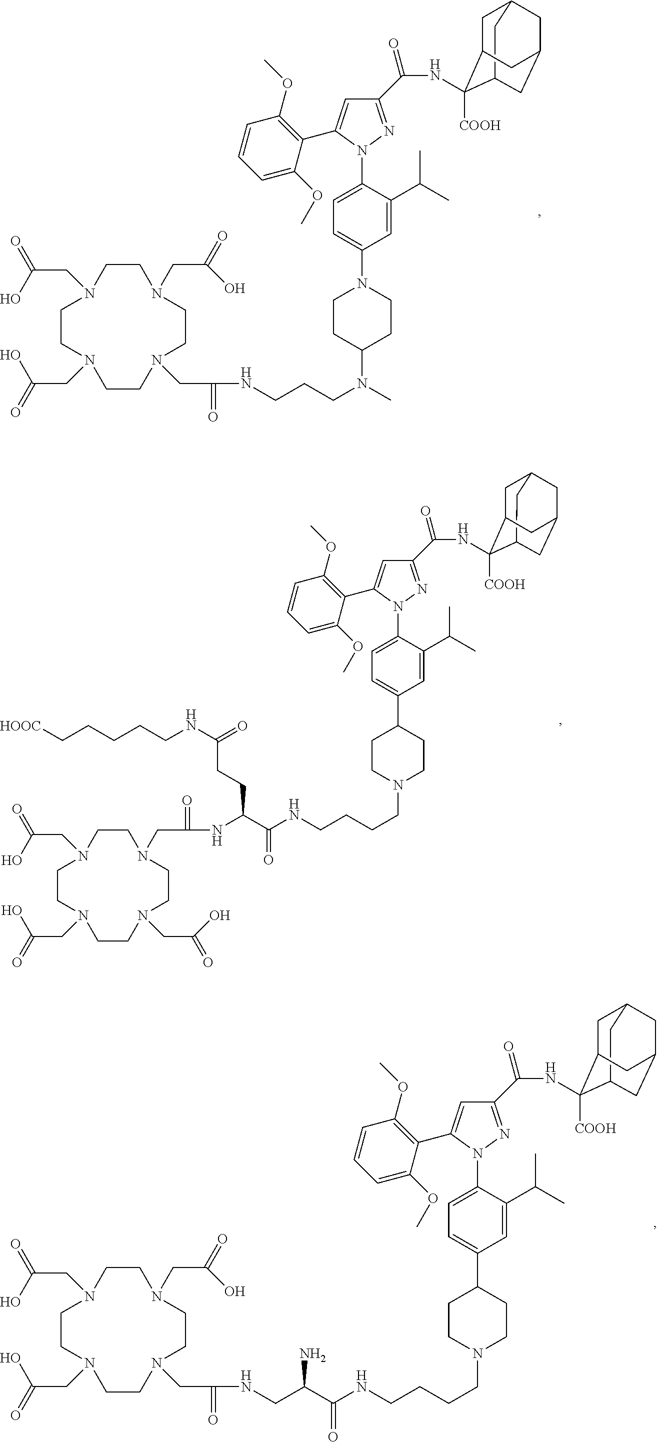 [SMILES: COc1cccc(OC)c1-c1cc(C(=O)NC2(C(=O)O)C3CC4CC(C3)CC2C4)nn1-c1ccc(N2CCC(N(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCC(=O)NCCCCCC(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](N)CNC(=O)CN3CCN(CC(=O)O)CCN(CC(=O)O)CCN(CC(=O)O)CC3)CC2)cc1C(C)C]

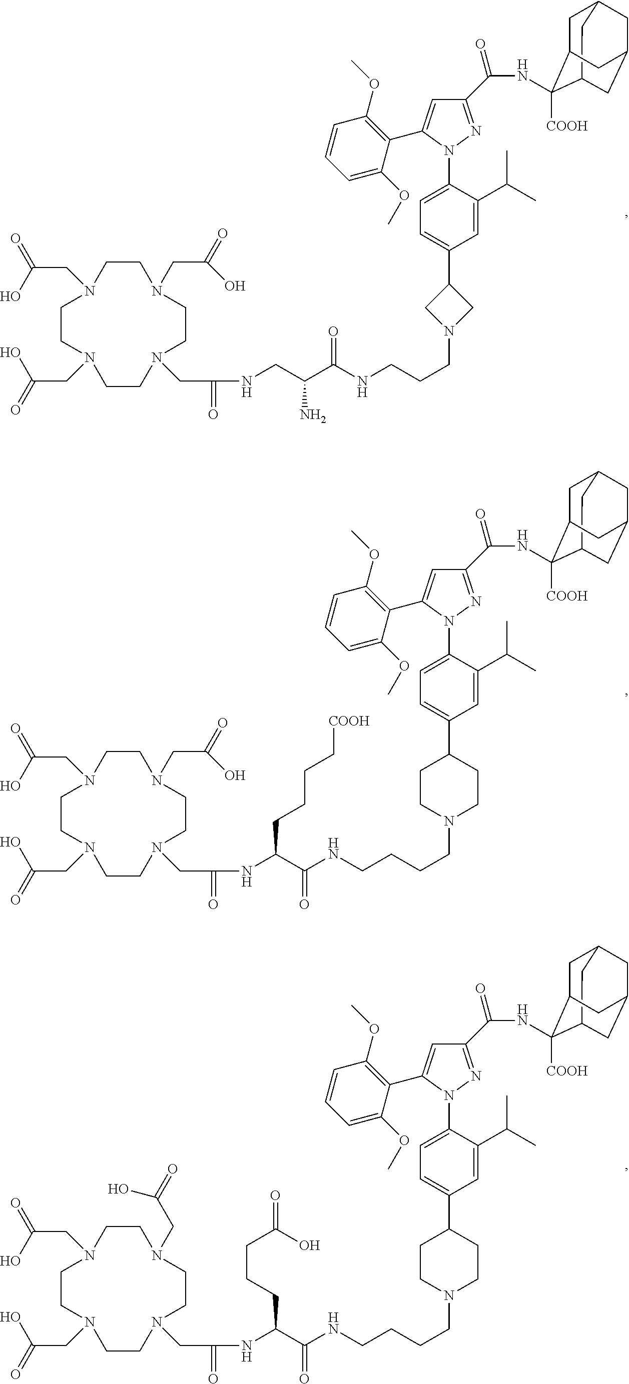 [SMILES: COc1cccc(OC)c1-c1cc(C(=O)NC2(C(=O)O)C3CC4CC(C3)CC2C4)nn1-c1ccc(C2CN(CCCNC(=O)[C@H](N)CNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCCCC(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCCC(=O)O)NC(=O)CN3CCN(CC(=O)O)CCN(CC(=O)O)CCN(CC(=O)O)CC3)CC2)cc1C(C)C]

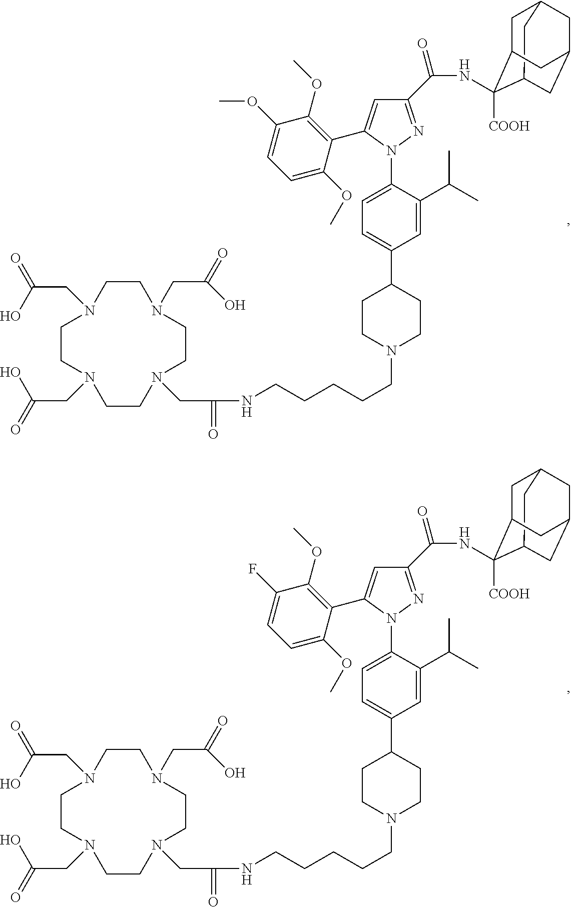 [SMILES: COc1ccc(OC)c(-c2cc(C(=O)NC3(C(=O)O)C4CC5CC(C4)CC3C5)nn2-c2ccc(C3CCN(CCCCCNC(=O)CN4CCN(CC(=O)O)CCN(CC(=O)O)CCN(CC(=O)O)CC4)CC3)cc2C(C)C)c1OC ; COc1ccc(F)c(OC)c1-c1cc(C(=O)NC2(C(=O)O)C3CC4CC(C3)CC2C4)nn1-c1ccc(C2CCN(CCCCCNC(=O)CN3CCN(CC(=O)O)CCN(CC(=O)O)CCN(CC(=O)O)CC3)CC2)cc1C(C)C]

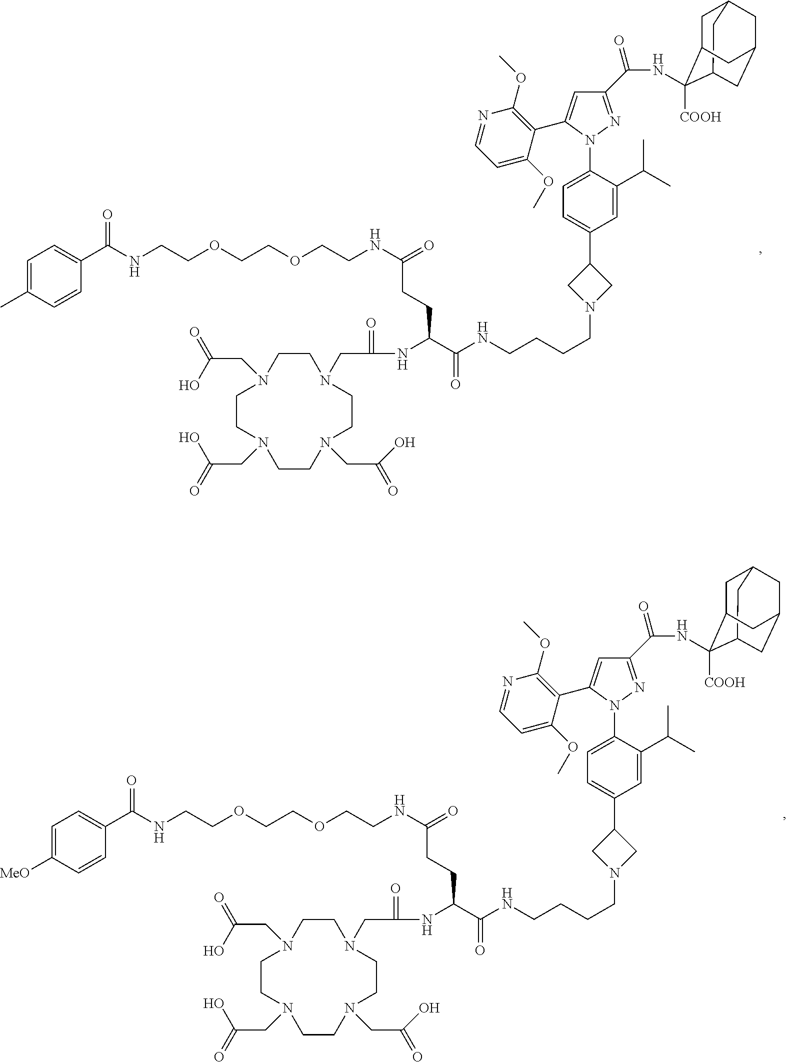 [SMILES: COc1ccnc(OC)c1-c1cc(C(=O)NC2(C(=O)O)C3CC4CC(C3)CC2C4)nn1-c1ccc(C2CN(CCCCNC(=O)[C@H](CCC(=O)NCCOCCOCCNC(=O)c3ccc(C)cc3)NC(=O)CN3CCN(CC(=O)O)CCN(CC(=O)O)CCN(CC(=O)O)CC3)C2)cc1C(C)C ; COc1ccc(C(=O)NCCOCCOCCNC(=O)CC[C@H](NC(=O)CN2CCN(CC(=O)O)CCN(CC(=O)O)CCN(CC(=O)O)CC2)C(=O)NCCCCN2CC(c3ccc(-n4nc(C(=O)NC5(C(=O)O)C6CC7CC(C6)CC5C7)cc4-c4c(OC)ccnc4OC)c(C(C)C)c3)C2)cc1]

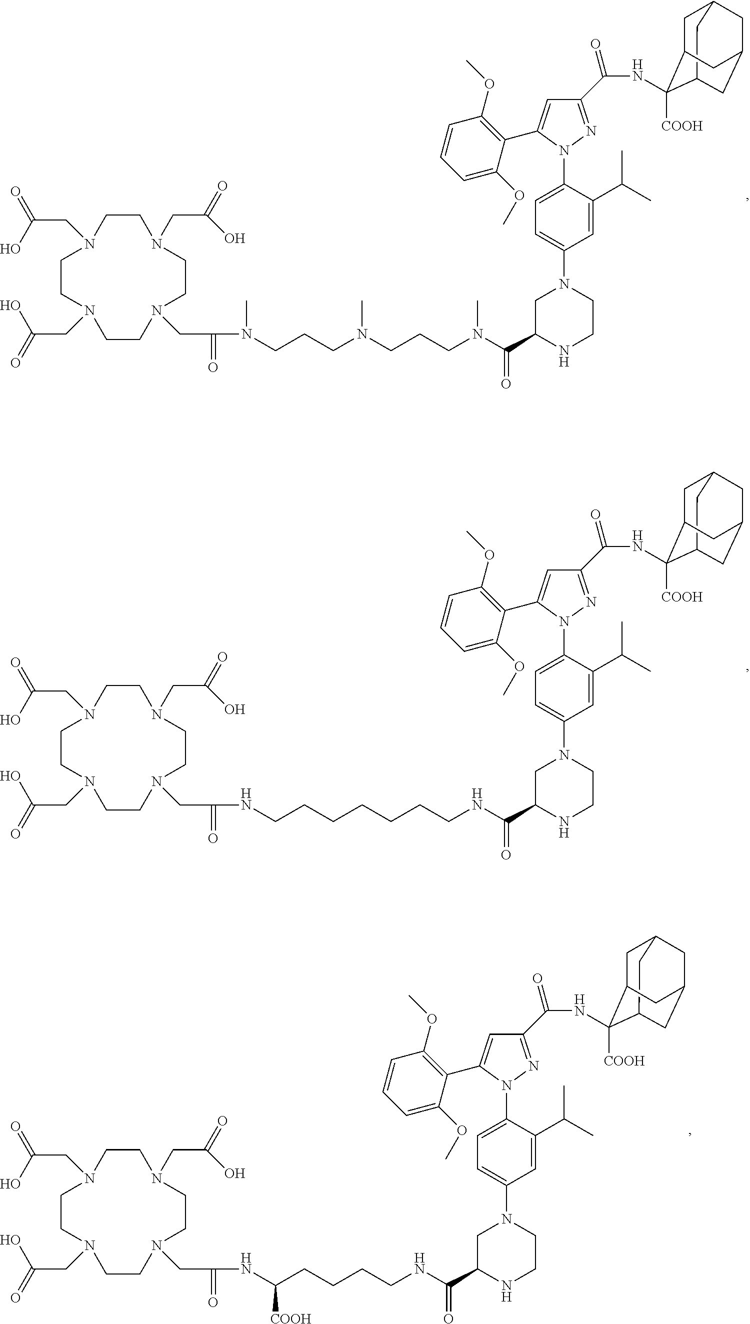 [SMILES: COc1cccc(OC)c1-c1cc(C(=O)NC2(C(=O)O)C3CC4CC(C3)CC2C4)nn1-c1ccc(N2CCN[C@@H](C(=O)N(C)CCCN(C)CCCN(C)C(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(N2CCN[C@@H](C(=O)NCCCCCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(N2CCN[C@@H](C(=O)NCCCC[C@H](NC(=O)CN3CCN(CC(=O)O)CCN(CC(=O)O)CCN(CC(=O)O)CC3)C(=O)O)C2)cc1C(C)C]

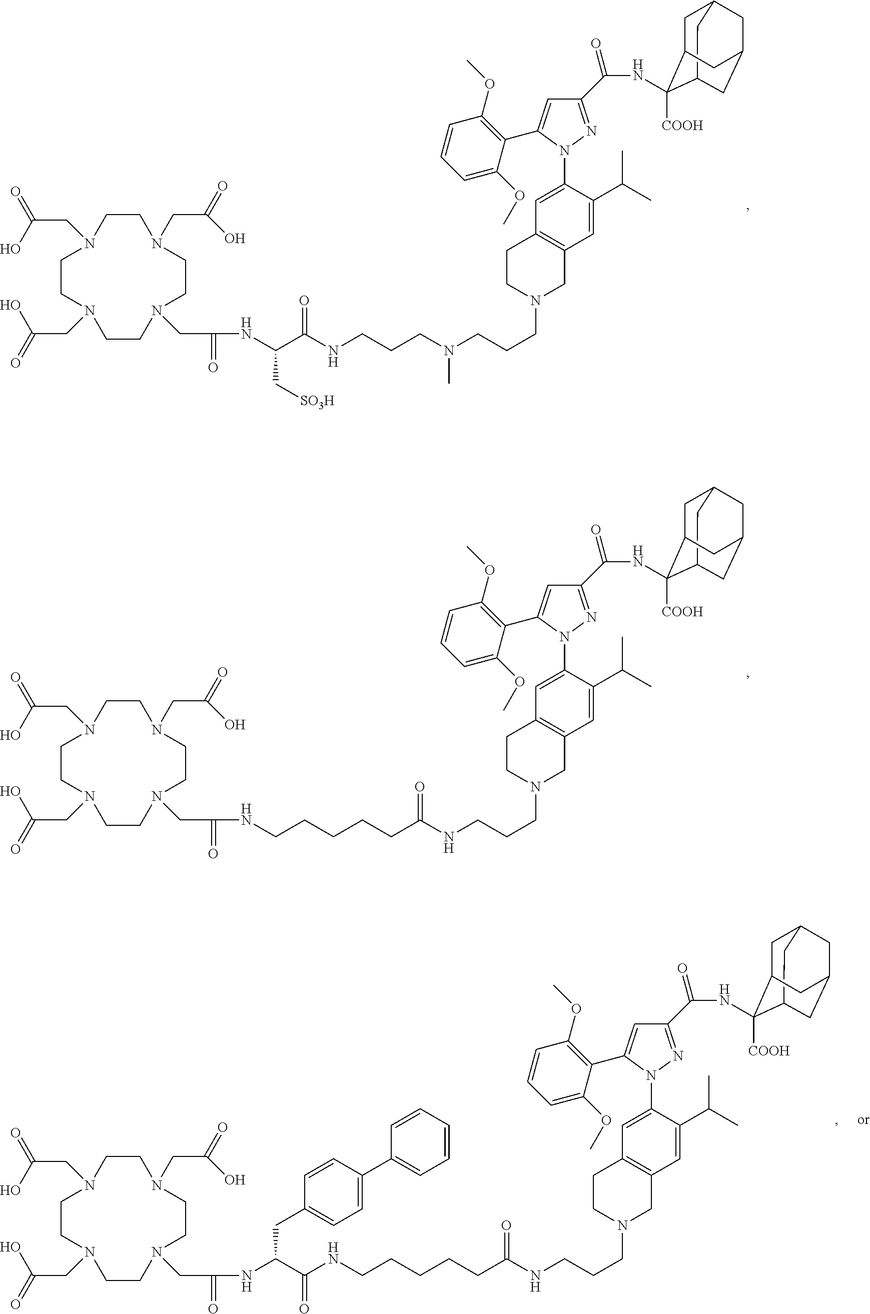 [SMILES: COc1cccc(OC)c1-c1cc(C(=O)NC2(C(=O)O)C3CC4CC(C3)CC2C4)nn1-c1cc2c(cc1C(C)C)CN(CCCN(C)CCCNC(=O)[C@H](CS(=O)(=O)O)NC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(CCCNC(=O)CCCCCNC(=O)CN1CCN(CC(=O)O)CCN(CC(=O)O)CCN(CC(=O)O)CC1)CC2 ; COc1cccc(OC)c1-c1cc(C(=O)NC2(C(=O)O)C3CC4CC(C3)CC2C4)nn1-c1cc2c(cc1C(C)C)CN(CCCNC(=O)CCCCCNC(=O)[C@@H](Cc1ccc(-c3ccccc3)cc1)NC(=O)CN1CCN(CC(=O)O)CCN(CC(=O)O)CCN(CC(=O)O)CC1)CC2]

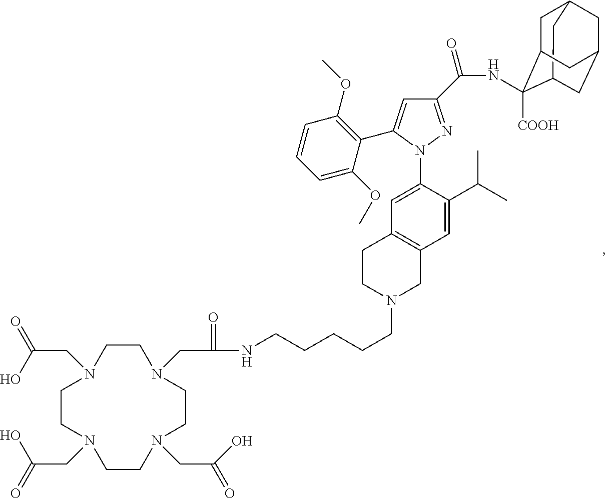 [SMILES: COc1cccc(OC)c1-c1cc(C(=O)NC2(C(=O)O)C3CC4CC(C3)CC2C4)nn1-c1cc2c(cc1C(C)C)CN(CCCCCNC(=O)CN1CCN(CC(=O)O)CCN(CC(=O)O)CCN(CC(=O)O)CC1)CC2]

or a radionuclide complex thereof.

Embodiment 86. The compound of any one of embodiments 1-85, or a pharmaceutically acceptable salt thereof, wherein: the radionuclide of the radionuclide complex is a lanthanide or an actinide.

Embodiment 87. The compound of any one of embodiments 1-85, or a pharmaceutically acceptable salt thereof, wherein: the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

Embodiment 88. The compound of any one of embodiments 1-85, or a pharmaceutically acceptable salt thereof, wherein: the radionuclide of the radionuclide complex is a diagnostic or therapeutic radionuclide.

Embodiment 89. The compound of any one of embodiments 1-85, or a pharmaceutically acceptable salt thereof, wherein: the radionuclide of the radionuclide complex is an Auger electron-emitting radionuclide, α-emitting radionuclide, β-emitting radionuclide, or γ-emitting radionuclide.

Embodiment 90. The compound of any one of embodiments 1-85, or a pharmaceutically acceptable salt thereof, wherein the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (^{99m}-Tc), or 195m-platinum (^{195m}Pt); or
an α-emitting radionuclide that is 225-actinium (²²⁵Ac), 213-bismuth (²¹³Bi), 223-Radium (²²³Ra), or 212-lead (²¹²Pb); or
a β-emitting radionuclide that is 90-yttrium (⁹⁰Y), 177-lutetium (177Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or
a γ-emitting radionuclide that is 60-cobalt (⁶¹Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

Embodiment 91. The compound of any one of embodiments 1-85, or a pharmaceutically acceptable salt thereof, wherein: the radionuclide of the radionuclide complex is 111-indium (¹¹¹In), 67-gallium (⁶⁷Ga), 68-gallium (⁶⁸Ga), 69-gallium (⁶⁹Ga), 71-gallium (71Ga), 225-actinium (²²⁵Ac), 175-lutetium (¹⁷⁵Lu), 177-lutetium (177Lu), 204-lead (²⁰⁴Pb), 206-lead (²⁰⁶Pb), 207-lead (²⁰⁷Pb), 208-lead (²⁰⁸Pb), 212-lead (212Pb), 63-copper (⁶³Cu), 64-copper (⁶⁴Cu), 65-copper (⁶⁵Cu), or 67-copper (⁶⁷Cu).

Embodiment 92. The compound of any one of embodiments 1-85, or a pharmaceutically acceptable salt thereof, wherein: the radionuclide of the radionuclide complex is 64-copper (⁶⁴Cu), 67-copper (⁶⁷Cu), 90-yttrium (⁹⁰Y), 111-indium (¹¹¹In), 67-gallium (⁶⁷Ga), 68-gallium (⁶⁸Ga), 225-actinium (²²⁵Ac), or 177-lutetium (¹⁷⁷Lu) or 212-lead (212Pb).

Embodiment 93. A pharmaceutical composition comprising a compound of any one of embodiments 1-92, or a pharmaceutically acceptable salt thereof, and at least one pharmaceutically acceptable excipient.

Embodiment 94. The pharmaceutical composition of embodiment 93, wherein the pharmaceutical composition is formulated for administration to a mammal by intravenous administration.

Embodiment 95. A method for the treatment of cancer comprising administering to a mammal with cancer an effective amount of a compound of any one of embodiments 1-92, or a pharmaceutically acceptable salt thereof.

Embodiment 96. The method of embodiment 95, wherein the cancer comprises tumors and the tumors overexpress Neurotensin Receptor 1 (NTSR1).

Embodiment 97. The method of embodiment 95 or embodiment 96, wherein the cancer is lung cancer, pancreatic cancer, colorectal cancer, head and neck cancers, breast cancer, prostate cancer, gastrointestinal cancer, or bone cancer.

Embodiment 98. A method of killing tumors in a mammal that overexpress Neurotensin Receptor 1 (NTSR1) comprising administering to the mammal a compound of any one of embodiments 1-97, or a pharmaceutically acceptable salt thereof, wherein the compound of any one of embodiments 1-92, or a pharmaceutically acceptable salt thereof, comprises a therapeutic radionuclide.

Embodiment 99. The method of embodiment 98, wherein the mammal has been diagnosed with lung cancer, pancreatic cancer, colorectal cancer, head and neck cancers, breast cancer, prostate cancer, gastrointestinal cancer, or bone cancer.

Embodiment 100. A method for identifying tumors expressing Neurotensin Receptor 1 (NTSRT) in a mammal comprising administering to the mammal a compound of any one of embodiments 1-92, or a pharmaceutically acceptable salt thereof; and performing positron emission tomography (PET) analysis, single-photon emission computerized tomography (SPECT), or magnetic resonance imaging (MRI); wherein the compound of any one of embodiments 1-92, or a pharmaceutically acceptable salt thereof, comprises a diagnostic radionuclide.

Embodiment 101. A method for the in vivo imaging of tissues or organs in a mammal with tumors expressing Neurotensin Receptor 1 (NTSR1) comprising administering to the mammal a compound of any one of embodiments 1-92, or a pharmaceutically acceptable salt thereof; and performing positron emission tomography (PET) analysis, single-photon emission computerized tomography (SPECT), or magnetic resonance imaging (MRI); wherein the compound of any one of embodiments 1-92, or a pharmaceutically acceptable salt thereof, comprises a diagnostic radionuclide.

Embodiment 102. A compound of Formula (D), or a pharmaceutically acceptable salt thereof:

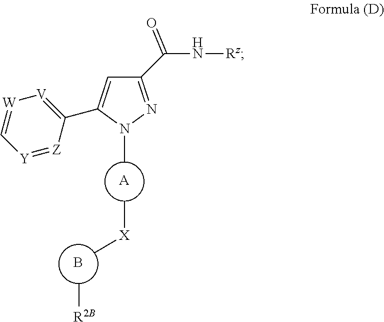

wherein:
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O);
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O);
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH(═N)OH, —NR⁵R⁶, —NO₂, —NR⁷C(═O)R⁸, —NR⁹C(═O)OR¹⁰, and —OC(═O)NR¹¹R¹²;
R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)—, ═S(═NH)(═NH), —CH═CH—C(═O)—, or —CH₂CH₂—C(═)—;
each R^{2A} is independently selected from the group consisting of hydrogen, —F, —Cl, —Br, —I, —OH, —O—C₁-C₆ alkyl, substituted or unsubstituted C₁-C₆ alkyl, C₁-C₆ fluoroalkyl, substituted or unsubstituted heteroalkyl, —CN, —CO₂H, —C(═O)NR^{3A}R^{4A}, —CH(═N)OH, —NR^{5A}R^{6A}, —NO₂, —NR^{7A}C(═O)R^{8A}—NR^{9A}C(═O)OR^{10A} and —OC(═O)NR^{11A}R^{12A} or two R^{2A} variables on the same or adjacent carbon atoms come together to form an unsubstituted C₃-C₆ cycloalkyl ring;
   R^{3A}, R^{4A}, R^{5A}, R^{6A}, R^{7A}, R^{8A}, R^{9A}, R^{10A}, R^{11A} and R^{12A} are each independently —H or —CH₃;
R^{2B} is hydrogen, —R^{b}, —U—R, or —NH—R^{d},
   R^{b} is hydrogen, C₁-C₆ alkyl or 6-membered heteroalkyl;
   U is —O— or —NH—;
   R^{c} is hydrogen or C₁-C₆ alkyl;
   R^{d} is C₁-C₆ alkyl;
W is —C(R^{r})— or —N— and Y is —C(R^{t})— or —N—; wherein R^{r} and R^{t} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—; wherein R^{x} and R^{y} are each independently selected from the group consisting of: —F, —Cl, —Br, —CF₃, —C₁-C₆ alkyl, —O—C₁-C₆ alkyl, —C₃-C₆ cycloalkyl, aryl and heteroaryl.
R^{z} is

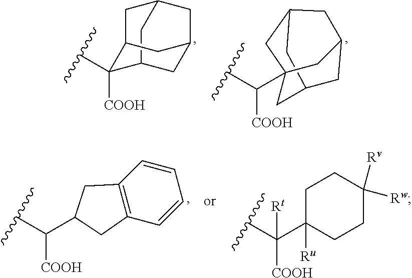

R^{v} and R^{w} are each independently —F, —Cl, or —CH₃ and R^{t} and R^{u} are each independently —C₁-C₄ alkyl; or
R^{v} is —F, —Cl, or —CH₃; R^{t} is —C₁-C₄ alkyl; and R^{u} and R^{w} come together with the carbon atoms to which they are attached to form a cyclopentyl or cyclohexyl ring.

Embodiment 103. The compound of embodiment 102, or a pharmaceutically acceptable salt thereof, wherein W is —N— and Y is —CH—.

Embodiment 104. The compound of embodiment 102, or a pharmaceutically acceptable salt thereof, wherein W and Y are —CH—.

Embodiment 105. The compound of any one of embodiments 102-104, or a pharmaceutically acceptable salt thereof, wherein: V is —C(R^{x})— and Z is —C(R^{y})—.

Embodiment 106. The compound of any one of embodiments 102-105, or a pharmaceutically acceptable salt thereof, wherein: R^{x} and R^{y} are each independently selected from the group consisting of: —F, —Cl, —Br, —CF₃, —C₁-C₆ alkyl, and —O—C₁-C₆ alkyl.

Embodiment 107. The compound of any one of embodiments 102-105, or a pharmaceutically acceptable salt thereof, wherein: R^{x} is —F, —Cl, —CF₃, —CH₃, —CH₂CH₃, —OCH₃, cyclopropyl, or 5 to 6-membered heteroaryl.

Embodiment 108. The compound of any one of embodiments 102-105, or a pharmaceutically acceptable salt thereof, wherein R^{x} is pyrimidinyl.

Embodiment 109. The compound of any one of embodiments 102-105 or 107-108, or a pharmaceutically acceptable salt thereof, wherein: R^{y} is —OCH³.

Embodiment 110. The compound of any one of embodiments 102-105, or a pharmaceutically acceptable salt thereof, wherein: R^{x} is —F, —Cl, —CF₃, —CH₃, —CH₂CH₃, —OCH₃, cyclopropyl, or pyrimidinyl and R^{y} is —OCH₃.

Embodiment 111. The compound of any one of embodiments 102-105, or a pharmaceutically acceptable salt thereof, wherein: R^{x} is —F, —Cl, or —OCH₃, and R^{y} is —OCH₃.

Embodiment 112. The compound of any one of embodiments 102-105, or a pharmaceutically acceptable salt thereof, wherein: R^{x} and R^{y} are —OCH₃.

Embodiment 113. The compound of embodiment 102, or a pharmaceutically acceptable salt thereof, wherein: W is —CH— or —N—; Y is —CH—; R^{x} is —OCH₃, and R^{y} is —OCH₃.

Embodiment 114. The compound of embodiment 102, or a pharmaceutically acceptable salt thereof, wherein:
V is —C(R^{x})—;
Z is —C(R^{y})—;
R^{x} is selected from the group consisting of: —F, —Cl, —CF₃, —C₁-C₆ alkyl, —O—C₁-C₆ alkyl, —C₃-C₆ cycloalkyl, and heteroaryl; and
R^{y} is —O—C₁-C₆ alkyl.

Embodiment 115. The compound of any one of embodiments 102-104, or a pharmaceutically acceptable salt thereof, wherein: V is —C(R^{x})—; Z is —C(R^{y})—; and R^{x} and R^{y} are each independently —O—C₁-C₆ alkyl.

Embodiment 116. The compound of any one of embodiments 102-115, or a pharmaceutically acceptable salt thereof, wherein R^{z} is

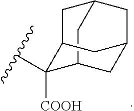 [SMILES: *C1(C(=O)O)C2CC3CC(C2)CC1C3]

Embodiment 117. The compound of any one of embodiments 102-115, or a pharmaceutically acceptable salt thereof, wherein R^{z} is

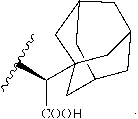 [SMILES: *[C@H](C(=O)O)C12CC3CC(CC(C3)C1)C2]

Embodiment 118. The compound of any one of embodiments 102-115, or a pharmaceutically acceptable salt thereof, wherein R^{z} is

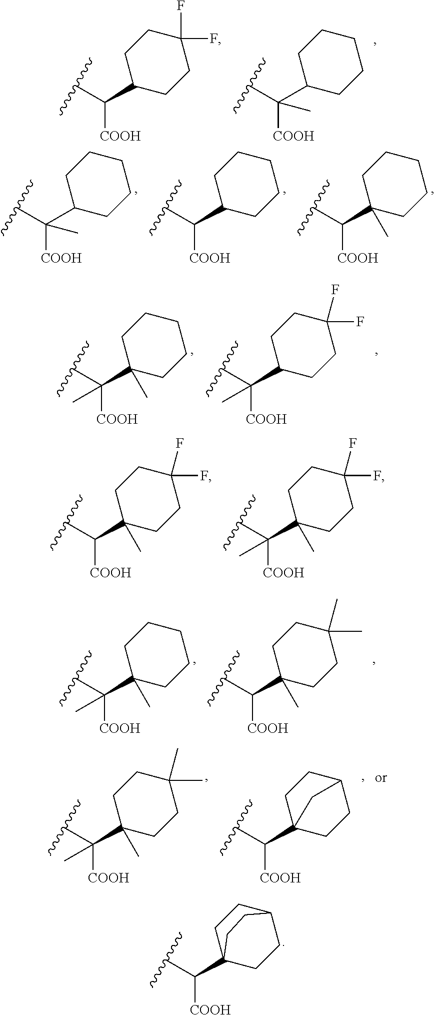

Embodiment 119. The compound of any one of embodiments 102-115, or a pharmaceutically acceptable salt thereof, wherein R^{z} is

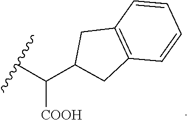 [SMILES: *C(C(=O)O)C1Cc2ccccc2C1]

Embodiment 120. The compound of embodiment 102, wherein the compound of Formula (D) is a compound of Formula (E), or a pharmaceutically acceptable salt thereof:

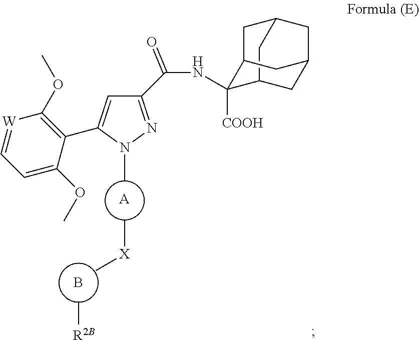

wherein.
Ring A is phenyl, naphthyl, or a bicyclic heteroaryl ring, and Ring B is piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O).
or Ring A is a partially unsaturated bicyclic heterocyclic ring, and Ring B is absent, piperidinyl, pyrrolidinyl, azetidinyl, piperazinyl, 1,2,3,6-tetrahydropyridinyl, 3,6-diazabicyclo[3.1.1]heptanyl, cyclobutyl, cyclopentyl, or cyclohexyl, wherein Ring B is optionally substituted with one or more R^{2A} substituents; and wherein any saturated carbon of Ring B is optionally substituted with oxo (═O).
wherein any saturated carbon of Ring A is optionally substituted with oxo (═O);
wherein each Ring A is optionally substituted with 1-6 R¹ substituents;
each R¹ is independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH—(═N)OH, —NR⁵R⁶, —NO₂, —NR⁷C(═O)R⁸, —NR⁹C(═O)OR¹⁰, and —OC(═O)NR¹¹R¹²;
R³, R⁴, R⁵, R⁶, R⁷, R⁸, R⁹, R¹⁰, R¹¹ and R¹² are each independently —H or —CH₃;
X is absent, —O—, —NH—, —S—, —S(═O)(═NH)—, ═S(═NH)(═NH), —CH═CH—C(═O)—, or —CH₂CH₂—C(═O)—;
each R^{2A} is independently selected from the group consisting of hydrogen, —F, —Cl, —Br, —I, —OH, —O—C₁-C₆ alkyl, substituted or unsubstituted C₁-C₆ alkyl, C₁-C₆ fluoroalkyl, substituted or unsubstituted heteroalkyl, —CN, —CO₂H, —C(═O)NR^{3A}R^{4A}, —CH(═N)OH, —NR^{5A}R^{6A}, —NO₂, —NR^{7A}C(═O)R^{8A}—NR^{9A}C(═O)OR^{10A}, and —OC(═O)NR^{11A}R^{12A};
R^{3A}, R^{4A}, R^{6A}, R^{6A}, R^{7A}R^{8A}, R^{9A}, R^{10A}, R^{11A} and R^{12A} are each independently —H or —CH₃;
R^{2B} is hydrogen, —R^{b}, —U—R^{c}, or —NH—R^{d},
   R^{b} is hydrogen, C₁-C₆ alkyl or 6-membered heteroalkyl;
   U is —O— or —NH—;
   R^{c} is hydrogen or C₁-C₆ alkyl;
   R^{d} is C₁-C₆ alkyl; and
W is —C(H)—, —C(F)—, —C(OMe)-, —C(OEt)-, or —N—.

Embodiment 121. The compound of any one of embodiments 102-120, or a pharmaceutically acceptable salt thereof, wherein Ring A is phenyl, naphthyl, or a bicyclic heteroaryl ring; wherein each Ring A is optionally substituted with 1-6 R¹ substituents.

Embodiment 122. The compound of any one of embodiments 102-120, or a pharmaceutically acceptable salt thereof, wherein Ring A is phenyl, naphthyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, or 1,5-naphthyridinyl; wherein each Ring A is optionally substituted with 1-6 R¹ substituents.

Embodiment 123. The compound of any one of embodiments 102-120, or a pharmaceutically acceptable salt thereof, wherein Ring A is phenyl, wherein Ring A is optionally substituted with 1-6 R¹ substituents.

Embodiment 124. The compound of any one of embodiments 102-120, or a pharmaceutically acceptable salt thereof, wherein Ring A is naphthyl, wherein Ring A is optionally substituted with 1-6 R¹ substituents.

Embodiment 125. The compound of any one of embodiments 102-120, or a pharmaceutically acceptable salt thereof, wherein Ring A is a partially unsaturated bicyclic heterocyclic ring; wherein any saturated carbon of Ring A is optionally substituted with oxo (═O) and wherein each Ring A is optionally substituted with 1-6 R¹ substituents.

Embodiment 126. The compound of any one of embodiments 102-120, or a pharmaceutically acceptable salt thereof, wherein Ring B is absent and Ring A is a partially unsaturated bicyclic heterocyclic ring selected from indolinyl, isoindolinyl, 1,2-dihydroisoquinolinyl, 1,2,3,4-tetrahydroquinolinyl, 1,2,3,4-tetrahydroisoquinolinyl, 1,2,3,4-tetrahydroquinoxalinyl, 1,2,3,4-tetrahydro-1,8-naphthyridine, 5,6,7,8-tetrahydro-1,7-naphthyridinyl, 5,6,7,8-tetrahydro-1,6-naphthyridinyl, 5,6,7,8-tetrahydro-1,5-naphthyridinyl, 1,2,3,4-tetrahydro-1,6-naphthyridinyl, 1,2,3,4-tetrahydro-2,6-naphthyridinyl, 1,2,3,4-tetrahydro-3,6-naphthyridinyl, or 1,2,3,4-tetrahydro-4,6-naphthyridinyl, or 2,3-dihydrobenzo[d]oxazolyl; wherein any saturated carbon of Ring A is optionally substituted with oxo (═O) and wherein each Ring A is optionally substituted with 1-6 R¹ substituents.

Embodiment 127. The compound of any one of embodiments 102-120, or a pharmaceutically acceptable salt thereof, wherein Ring B is absent and Ring A is 1,2,3,4-tetrahydroisoquinolinyl, wherein Ring A is optionally substituted with 1-6 R¹ substituents.

Embodiment 128. The compound of any one of embodiments 102-127, or a pharmaceutically acceptable salt thereof, wherein each R¹ is independently selected from the group consisting of —F, —Cl, —Br, —I, —OH, —CF₃, —CN, —CO₂H, —CO₂C—3, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

Embodiment 129. The compound of any one of embodiments 102-127, or a pharmaceutically acceptable salt thereof, wherein each R¹ is independently selected from the group consisting of —F, —Cl, —Br, —I, —OH and —C₁-C₆ alkyl.

Embodiment 130. The compound of any one of embodiments 102-127, or a pharmaceutically acceptable salt thereof, wherein R¹ is —CH(CH₃)₂.

Embodiment 131. The compound of any one of embodiments 102-130, or a pharmaceutically acceptable salt thereof, wherein X is a bond.

Embodiment 132. The compound of any one of embodiments 102-130, or a pharmaceutically acceptable salt thereof, wherein X is —O— or —NH—.

Embodiment 133. The compound of any one of embodiments 102-130, or a pharmaceutically acceptable salt thereof, wherein X is —S(═O)(═NH)— or ═S(═NH)(═NH)—.

Embodiment 134. The compound of any one of embodiments 102-133, or a pharmaceutically acceptable salt thereof, wherein Ring B is piperidinyl, pyrrolidinyl, or azetidinyl, and R^{2B} is hydrogen, —R^{b}, —U—R^{c}, or —NH—R^{d}; wherein Ring B is optionally substituted with one or more R^{2A} substituents; and wherein any saturated carbon of Ring B is optionally substituted with oxo (═O).

Embodiment 135. The compound of any one of embodiments 102-133, or a pharmaceutically acceptable salt thereof, wherein Ring B is substituted or unsubstituted piperazinyl and R^{2B} is hydrogen, —R^{b}, —U—R^{c}, or —NH—R^{d}; wherein Ring B is optionally substituted with one or more R^{2A} substituents.

Embodiment 136. The compound of any one of embodiments 102-133, or a pharmaceutically acceptable salt thereof, wherein Ring B is piperidinyl and R^{2B} is hydrogen, —R^{b}, —U—R^{c}, or —NH—R^{d}.

Embodiment 137. The compound of any one of embodiments 1-35, or a pharmaceutically acceptable salt thereof, wherein each R^{2A} is independently selected from the group consisting of —F, substituted or unsubstituted C₁-C₆ alkyl, —CF₃, or —CN.

Embodiment 138. The compound of any one of embodiments 102-137, or a pharmaceutically acceptable salt thereof, wherein each R^{2A} is independently —F or substituted or unsubstituted —C₁-C₆ alkyl.

Embodiment 139. The compound of any one of embodiments 102-137, or a pharmaceutically acceptable salt thereof, wherein R^{2A} is —CH₂NH₂.

Embodiment 140. The compound of any one of embodiments 102-133, or a pharmaceutically acceptable salt thereof, wherein —X-Ring B is absent,

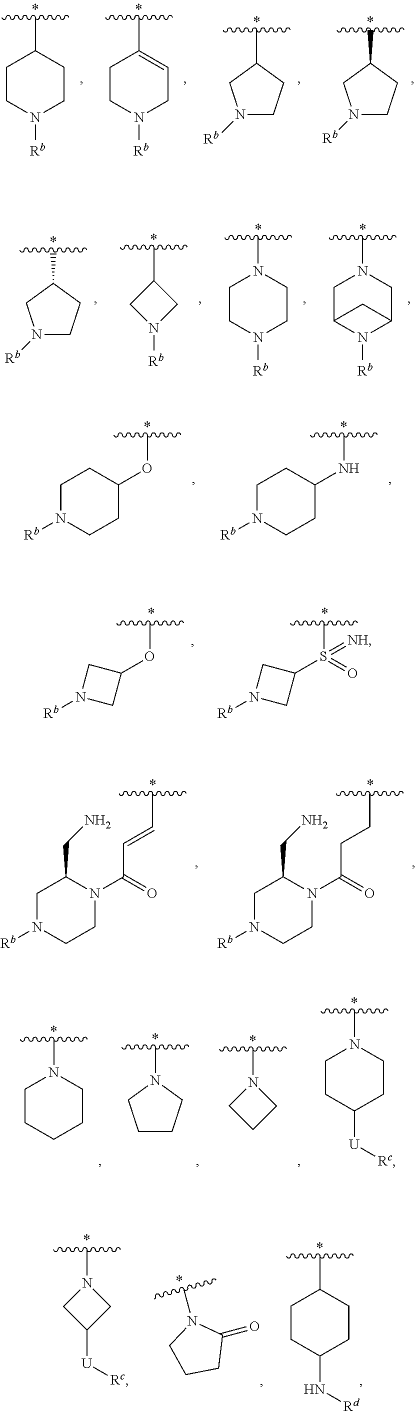

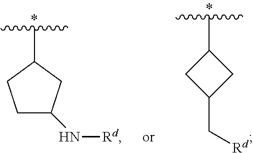

U is —O— or —NH—; Rh is hydrogen, C₁-C₆ alkyl or 6-membered heteroalkyl; R^{c} is hydrogen or C₁-C₆ alkyl; and R^{d} is C₁-C₆ alkyl; wherein * denotes the attachment point of —X-Ring B— to Ring A.

Embodiment 141. The compound of any one of embodiments 102-133, or a pharmaceutically acceptable salt thereof, wherein embodiments, Ring A is

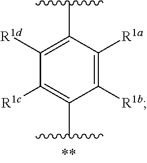

wherein ** denotes the attachment point of Ring A to X; and —X-Ring B— is

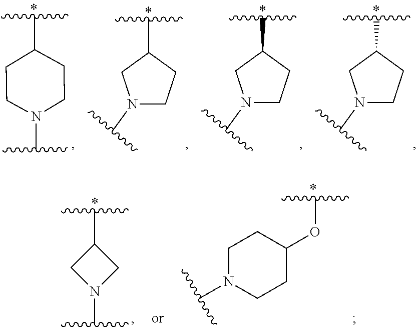 [SMILES: *C1CCN(*)CC1 ; *C1CCN(*)C1 ; *[C@H]1CCN(*)C1 ; *[C@@H]1CCN(*)C1 ; *C1CN(*)C1 ; *OC1CCN(*)CC1]

wherein * denotes the attachment point of —X-Ring B— to Ring A.

Embodiment 142. The compound of embodiment 102 or 120, or a pharmaceutically acceptable salt thereof, wherein Ring A-X-Ring B is:

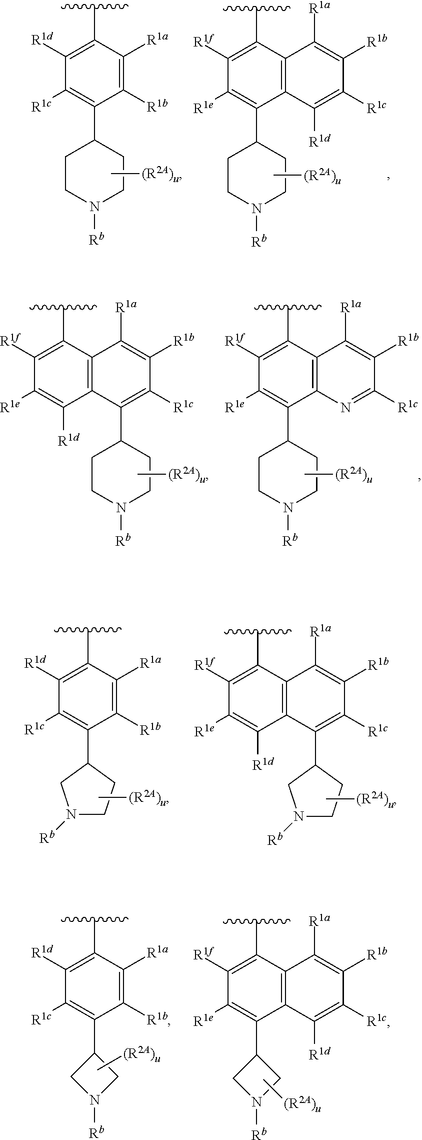

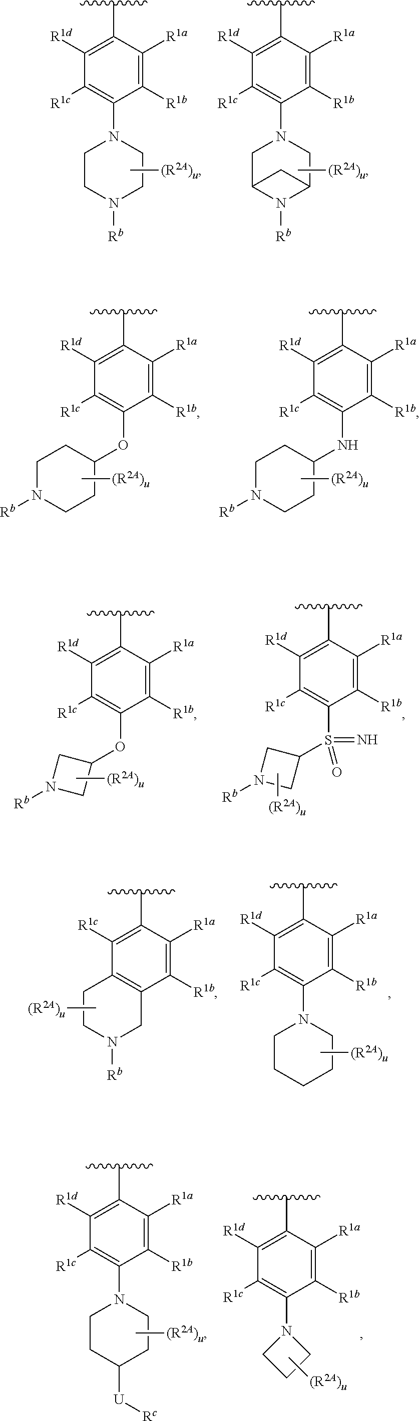

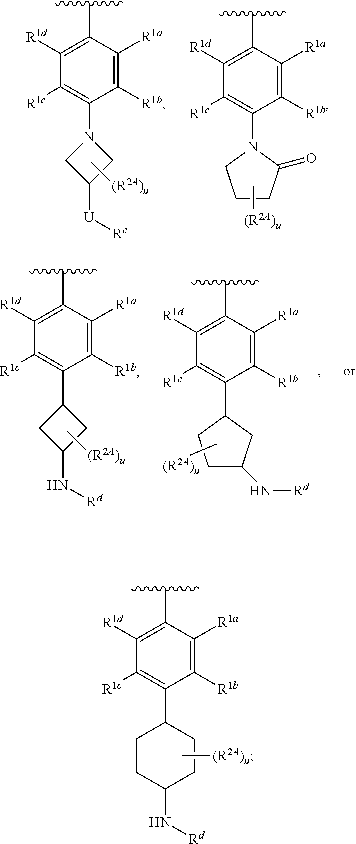

wherein R^{1a}, R^{1b}, R^{1c}, R^{1d}, R^{1e}, and R^{1f} are each independently selected from the group consisting of hydrogen, —F, —CI, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; U is —O— or —NH—; R^{b} is hydrogen, substituted or unsubstituted C₁-C₆ alkyl or substituted or un substituted 6-membered heteroalkyl; R^{c} is hydrogen or C₁-C₆ alkyl; R^{d} is C₁-C₆ alkyl; R^{2A} is hydrogen, —F, —Cl, —Br, —I, —OH, —O—C₁-C₆ alkyl, unsubstituted C₁-C₆ alkyl, or C₁-C₆ fluoroalkyl; and u is 0, 1, 2, 3, or 4.

Embodiment 143. The compound of embodiment 102 or 120, or a pharmaceutically acceptable salt thereof, wherein Ring A-X-Ring B is:

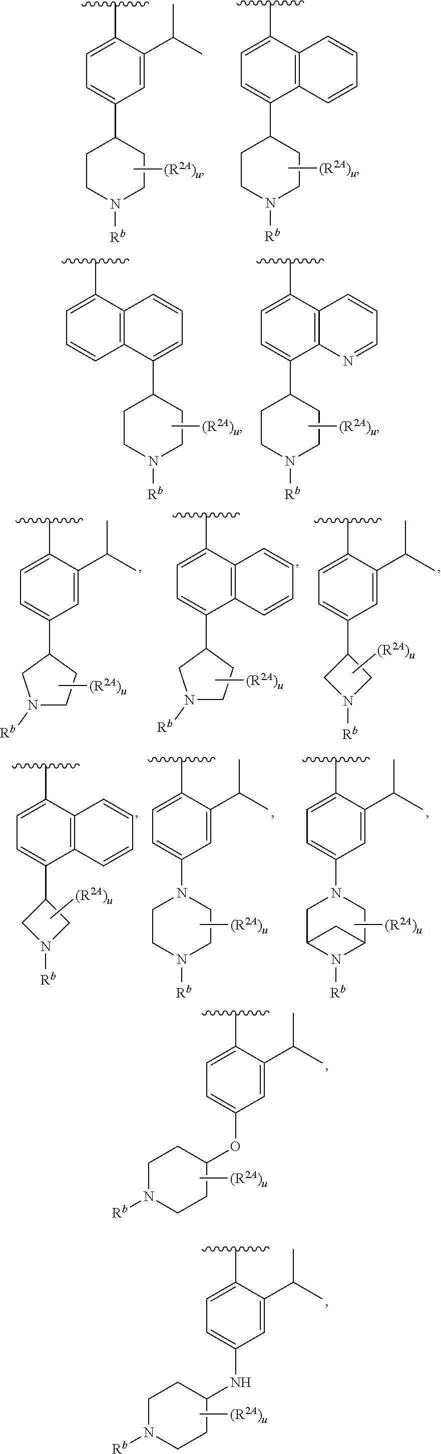

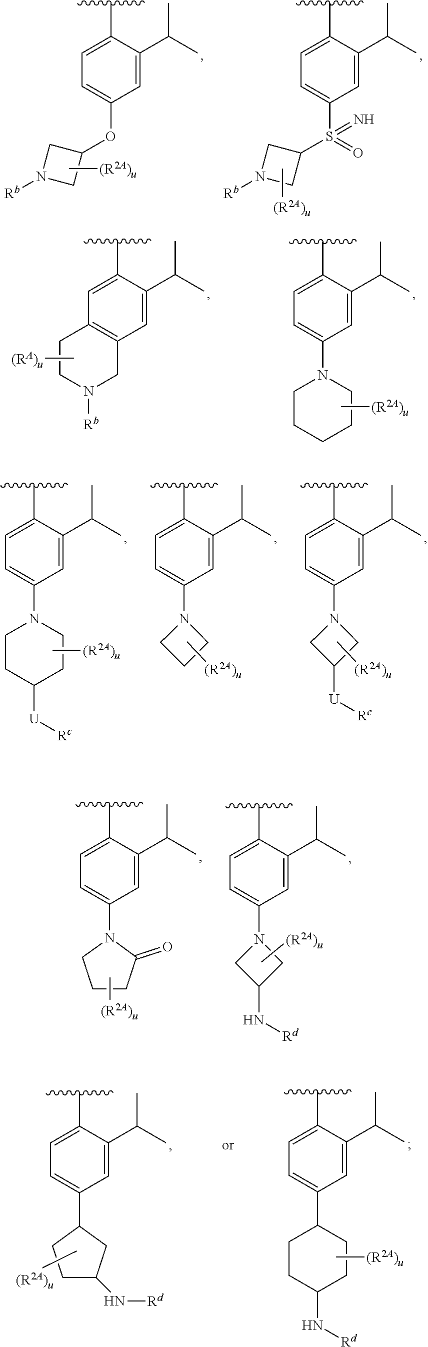

wherein U is —O— or —NH—; Rbis hydrogen, substituted or unsubstituted C₁-C₆ alkyl or substituted or unsubstituted 6-membered heteroalkyl; R^{c} is hydrogen or C₁-C₆ alkyl; R^{d} is C₁—C₆ alkyl; R^{2A} is hydrogen, —F, —Cl, —Br, —I, —OH, —O—C₁-C₆ alkyl, unsubstituted C₁-C₆ alkyl, or C₁-C₆ fluoroalkyl; and u is 0, 1, 2, 3, or 4.

Embodiment 144. The compound of embodiment 102 or 120, or a pharmaceutically acceptable salt thereof, wherein Ring A-X-Ring B is:

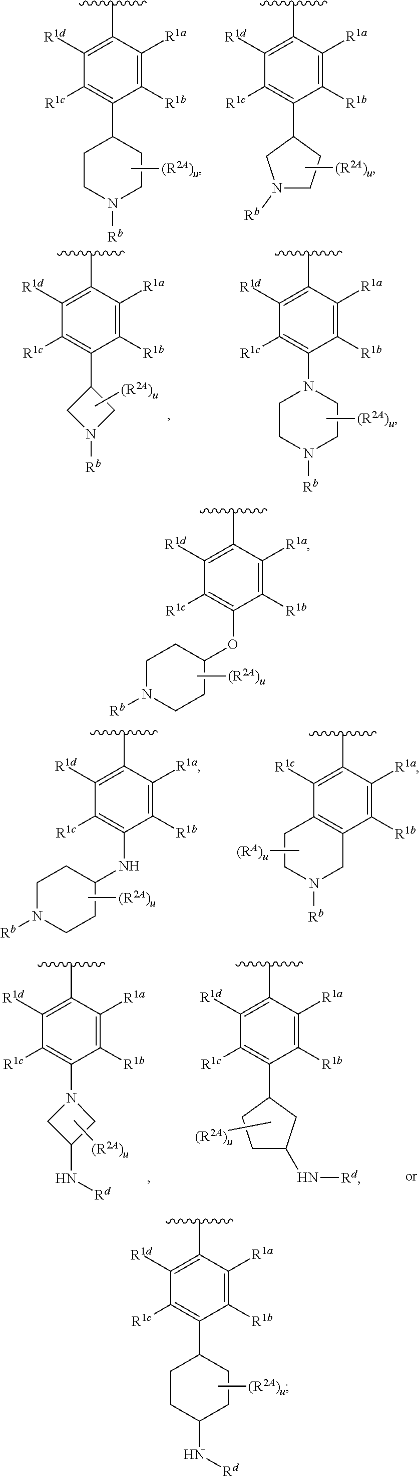

wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl; U is —O— or —NH—; R^{b} is hydrogen, substituted or unsubstituted C₁-C₆ alkyl or substituted or unsubstituted 6-membered heteroalkyl;
R^{c} is hydrogen or C₁-C₆ alkyl; R^{d} is C₁-C₆ alkyl; R^{2A} is hydrogen, —F, —Cl, —Br, —I, —OH, —O—C₁-C₆ alkyl, unsubstituted C₁-C₆ alkyl, or C₁-C₆ fluoroalkyl; and u is 0, 1, 2, 3, or 4.

Embodiment 145. The compound of embodiment 102 or 120, or a pharmaceutically acceptable salt thereof, wherein Ring A-X-Ring B is:

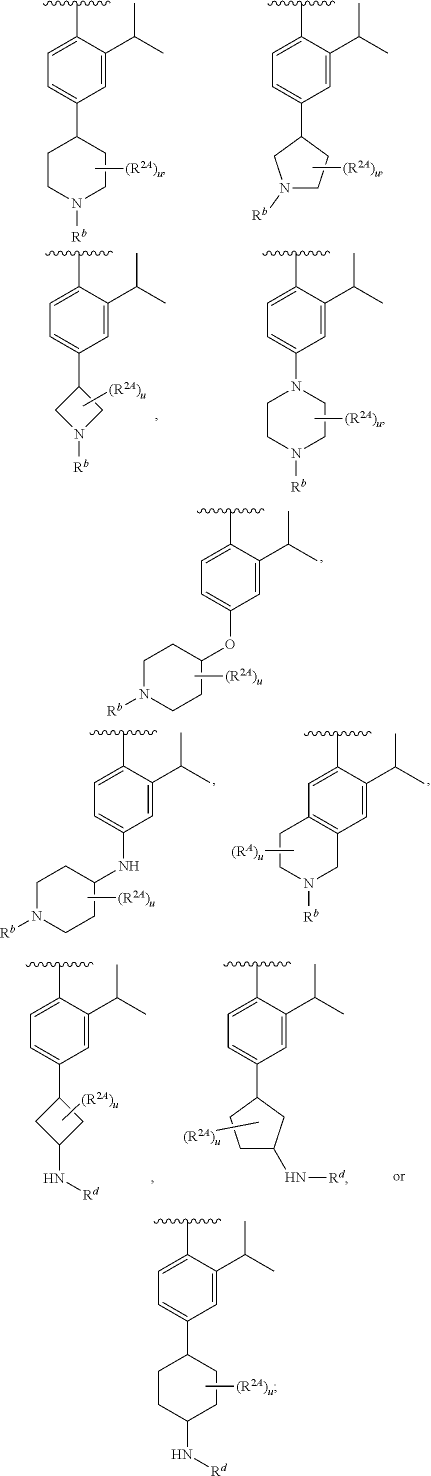

wherein U is —O— or —NH—; R^{b} is hydrogen, substituted or unsubstituted C₁-C₆ alkyl or substituted or unsubstituted 6-membered heteroalkyl; R^{c} is hydrogen or C₁-C₆ alkyl; R^{d} is C₁-C₆ alkyl; R^{2A} is hydrogen, —F, —Cl, —Br, —I, —OH, —O—C₁-C₆ alkyl, unsubstituted C₁-C₆ alkyl, or C₁-C₆ fluoroalkyl; and u is 0, 1, 2, 3, or 4.

Embodiment 146. The compound of embodiment 102, or a pharmaceutically acceptable salt thereof, wherein the compound has the following structure:

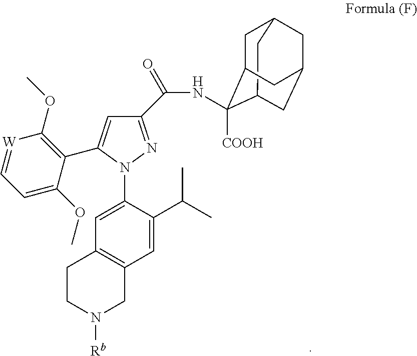

Embodiment 147. The compound of embodiment 102, or a pharmaceutically acceptable salt thereof, wherein the compound has the following structure:

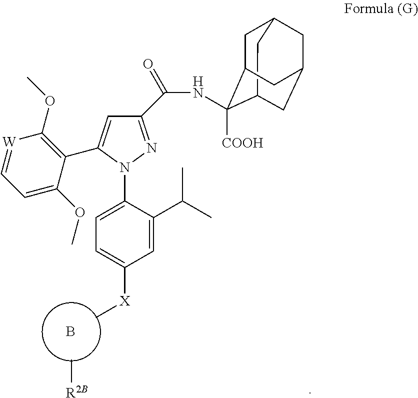

Embodiment 148. The compound of embodiment 102, or a pharmaceutically acceptable salt thereof, wherein the compound has the following structure:

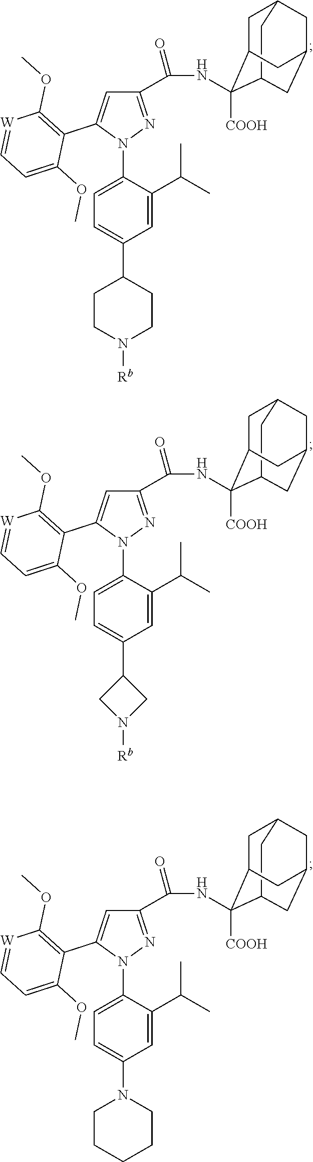

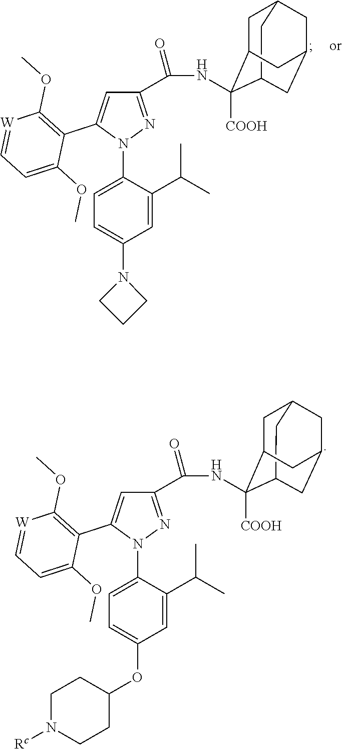

Embodiment 149. The compound of embodiment 102, or a pharmaceutically acceptable salt thereof, wherein the compound has the following structure:

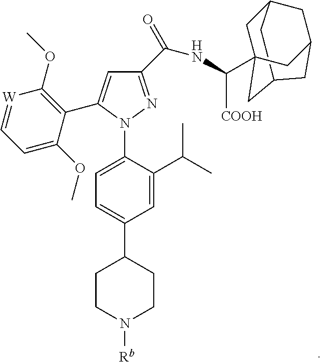

Embodiment 150 The compound of embodiment 102, or a pharmaceutically acceptable salt thereof, wherein the compound has the following structure:

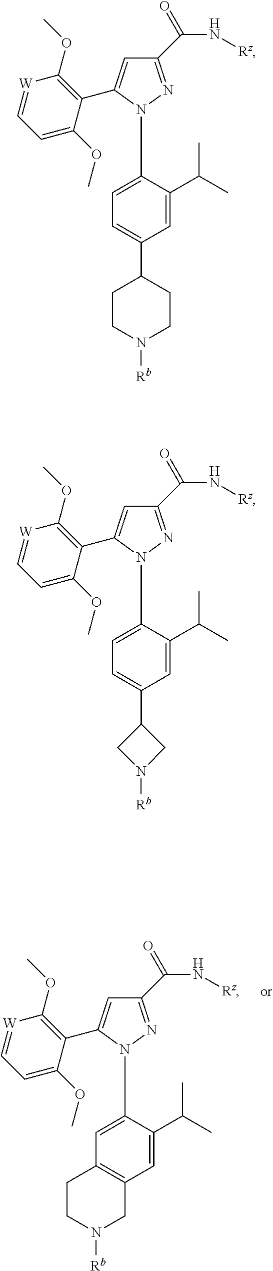

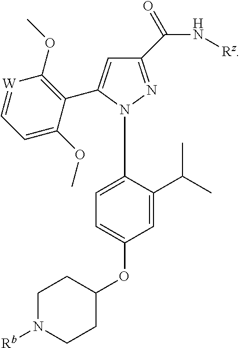

Embodiment 151. The compound of embodiment 102, or a pharmaceutically acceptable salt thereof, wherein the compound has one of the following structures:

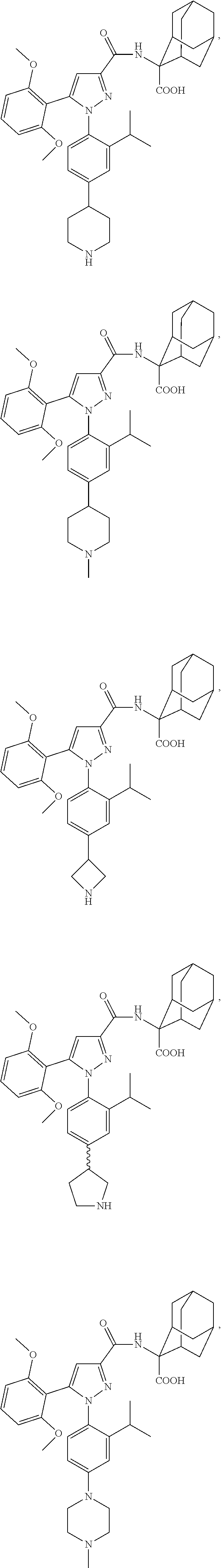

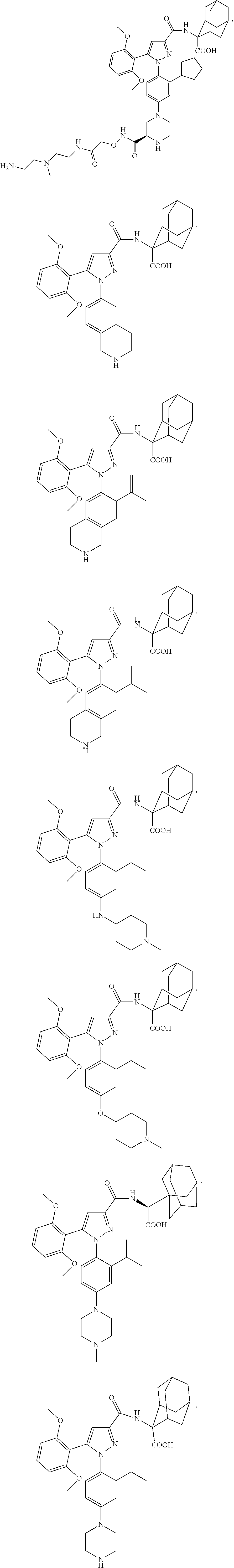

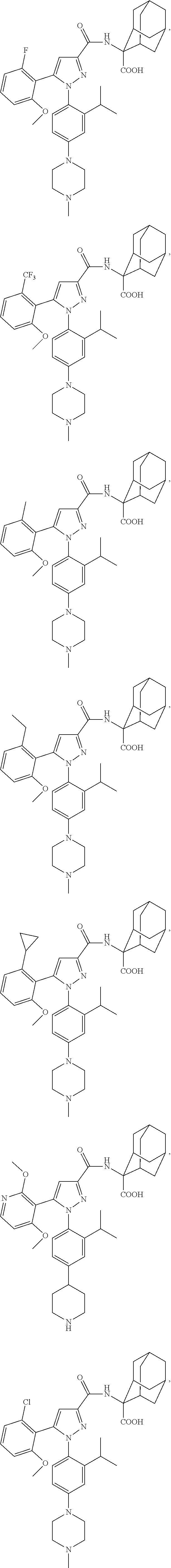

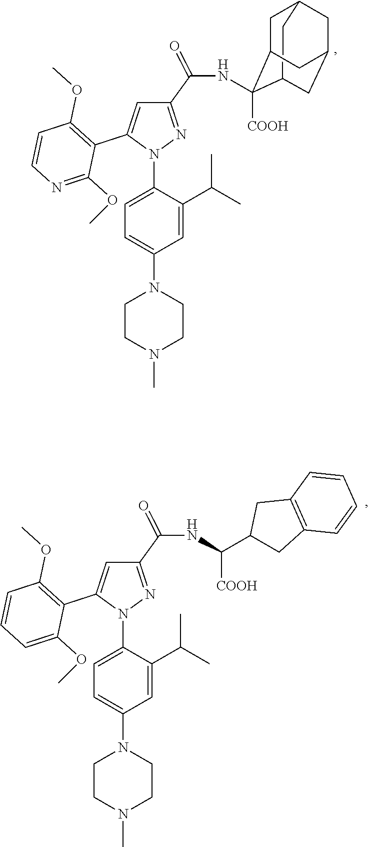 [SMILES: COc1ccnc(OC)c1-c1cc(C(=O)NC2(C(=O)O)C3CC4CC(C3)CC2C4)nn1-c1ccc(N2CCN(C)CC2)cc1C(C)C ; "," ; COc1cccc(OC)c1-c1cc(C(=O)N[C@H](C(=O)O)C2Cc3ccccc3C2)nn1-c1ccc(N2CCN(C)CC2)cc1C(C)C ; ","]

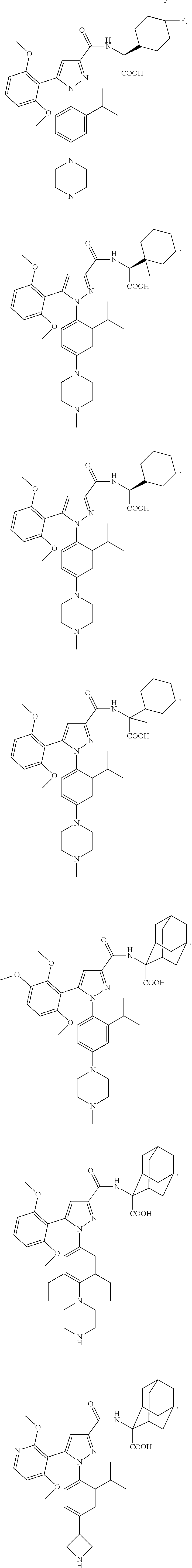

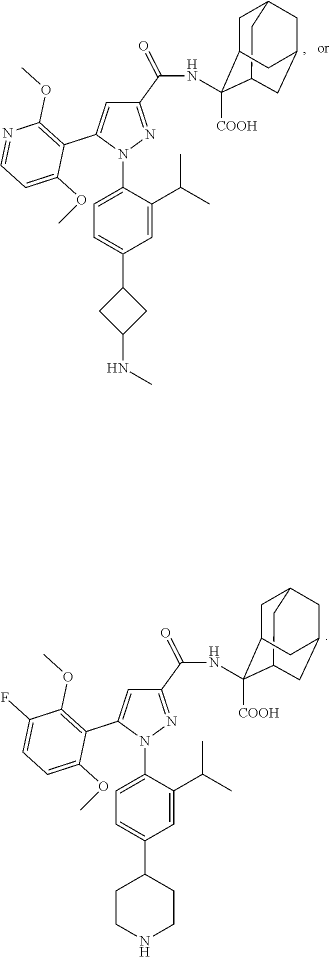 [SMILES: CNC1CC(c2ccc(-n3nc(C(=O)NC4(C(=O)O)C5CC6CC(C5)CC4C6)cc3-c3c(OC)ccnc3OC)c(C(C)C)c2)C1 ; "," ; COc1ccc(F)c(OC)c1-c1cc(C(=O)NC2(C(=O)O)C3CC4CC(C3)CC2C4)nn1-c1ccc(C2CCNCC2)cc1C(C)C ; "."]

Embodiment 152. A pharmaceutical composition comprising a compound of any one of embodiments 102-151, or a pharmaceutically acceptable salt thereof, and at least one pharmaceutically acceptable excipient.

Embodiment 153. The pharmaceutical composition of embodiment 152, wherein the pharmaceutical composition is formulated for administration to a mammal by oral administration.

Embodiment 154. A method for the treatment of a disease comprising administering to a mammal with the disease an effective amount of a compound of any one of embodiments 102-151, or a pharmaceutically acceptable salt thereof.

Embodiment 155. The method of embodiment 154 wherein the disease is a psychological disorder, a movement disorder, a gastrointestinal disorder, a cardiovascular disorder, a metabolic disorder, or pain management.

Embodiment 156. The method of embodiment 155, wherein the psychological disorder is psychosis or schizophrenia.

Embodiment 157. The method of embodiment 155, wherein the movement disorder is Parkinson's disease.

Embodiment 158. The method of embodiment 155, wherein the gastrointestinal disorder is irritable bowel syndrome (IBS), diarrhea, colitis, ulcers, tumors of the GI tract, dyspepsia, pancreatitis, or esophagitis.

### EXAMPLES

The following examples are provided for illustrative purposes only and not to limit the scope of the claims provided herein.

### Abbreviations

ACN or MeCN or CH₃CN: acetonitrile;
AcOH: acetic acid;
Boc₂O: di-tert-butyl dicarbonate;
brine: saturated NaCl solution;
BOP: Benzotriazole-1-yl-oxy-tris-(dimethylamino)-phosphonium hexafluorophosphate;
Cbz-Cl: Benzyl chloroformate;
Cbz-OSu: N-(Benzyloxycarbonyloxy)succinimide or benzyl (2,5-dioxopyrrolidin-1-yl) carbonate;
CH₃I: methyl iodide;
Cs₂CO₃: cesium carbonate;
DBU: 1,8-diazabicyclo(5.4.0)undec-7-ene;
DCC: dicyclohexylcarbodiimide;
DCE: dichloroethane;
DCM: dichloromethane;
DIEA or DIPEA: N,N-diisopropylethylamine;
DMAc: dimethylacetamide;
DMF: dimethylformamide;
DMP: Dess-Martin periodinane;
DMSO: dimethyl sulfoxide;
DOTA: 2,2′,2″,2′″-(1,4,7,10-tetraazacyclododecane-1,4,7,10-tetrayl)tetraacetic acid or 1,4,7,10-tetraazacyclododecane-1,4,7,10-tetraacetic acid;
DOTA-OSu: 2,2′,2″-(10-(2-((2,5-dioxopyrrolidin-1-yl)oxy)-2-oxoethyl)-1,4,7,10-tetraazacyclododecane-1,4,7-triyl)triacetic acid;
(4,4′-Dtbbpy)NiCl₂: [4,4′-bis(1,1-dimethylethyl)-2,2′-bipyridine]nickel (II) dichloride;
EDC or EDCL: (1-ethyl-3-(3-dimethylaminopropyl)carbodiimide hydrochloride);
Et₃N: triethylamine;
EtOAc or EA: ethyl acetate;
Et₂O: diethyl ether;
HATU: 1-[Bis(dimethylamino)methylene]-1H-1,2,3-triazolo[4,5-b]pyridinium 3-oxide hexafluorophosphate;
HCl: hydrochloric acid or hydrochloride; H₂O: water;
N₂OH NH₂C(═O)NH₂: carbamide peroxide;
HBTU: N,N,N′,N″-Tetramethyl-O—(H-benzotriazol-1-yl)uronium hexafluorophosphate;
HOBt: hydroxybenzotriazole;
HOPO: 2-Pyridinol 1-oxide;
HNO₃: nitric acid;
HPLC: high-performance liquid chromatography;
InCl₃: indium trichloride;
IPA: isopropyl alcohol;
K₂CO₃: potassium carbonate;
KOAc: potassium acetate;
KSCN: potassium thiocyanate;
LCMS: Liquid chromatography-mass spectrometry;
LiOH: lithium hydroxide;
LuCl₃: lutetium (III) chloride;
MeOH: methanol;
Me₃SnOH: trimethyltin hydroxide;
MPLC: Medium pressure liquid chromatography;
MS: mass spectrometry;
NaBH₄: sodium borohydride or sodium tetrahydroborate;
NaCNBH₃: sodium cyanoborohydride;
NaH: sodium hydride;
NaHCO₃: sodium bicarbonate;
NaHSO₄: sodium bisulfite;
Nat: sodium iodide;
NaNO₂: sodium nitrite;
NaOMe: sodium methoxide;
(NH₄)₂CO₃: ammonium carbonate;
NH₄Cl: ammonium chloride;
NHS: N-hydroxysuccinimide;
NIS: N-iodosuccinimide;
NaOH: sodium hydroxide;
Na₂S: sodium sulfide;
Na₂SO₄: sodium sulfate;
Pd₂(dba)₃: Tris(dibenzylideneacetone)dipalladium(0);
Pd(amphos)Cl₂: bis(di-tert-butyl(4-dimethylaminophenyl)phosphine)dichloropalladium(II);
Pd(dppf)Cl₂: [1,1′-Bis(diphenylphosphino)ferrocene]dichloropalladium(II);
Pd(dtbpf)Cl₂: [1,1′-Bis(di-tert-butylphosphino)ferrocene]dichloropalladium(II);
Pd(OH)₂/C: palladium hydroxide on carbon;
Pd(OAc)₂: palladium (II) acetate;
Pd(PPh3)₄: tetrakis(triphenylphosphine)palladium(0);
Prep-HPLC: preparative high-performance liquid chromatography;
RockPhos: 2-(di-t-butylphosphino)-3-methoxy-6-methyl-2′,4′,6′-tri-i-propyl-1,1′-biphenyl;
RP-HPLC: reversed-phase high-performance liquid chromatography;
RuPhos: 2-dicyclohexylphosphino-2′,6′-diisopropoxybiphenyl;
SnCl₂: stannous chloride or dichlorostannane;
TBAI: tetrabutylammonium iodide;
TFA: trifluoroacetic acid;
THF: tetrahydrofuran;
XantPhos: (9,9-Dimethyl-9H-xanthene-4,5-diyl)bis(diphenylphosphane)
XPhos: 2-(dicyclohexylphosphanyl)-2′,4′,6′-tris(isopropyl)biphenyl;
XPhos Pd G3: (2-Dicyclohexylphosphino-2′,4′,6′-triisopropyl-1,1′-biphenyl)[2-(2′-amino-1,1′-biphenyl)]palladium(II) methanesulfonate;
ZnCl₂: zinc(II) chloride;
rt: room temperature;
hrs: hours; h or hr: hour;
min: minute;
mg: milligrams; kg: kilograms;
mL or ml: milliliter;
Eq: equivalents;
mmol: millimole; mol: moles;
UV: ultraviolet;
v/v: volume/volume.

Reagents were obtained from commercial suppliers and used without further purification, unless otherwise noted.

Reactions were carried out in Syro II (Biotage) and/or manual shaker using Fmoc chemistry, unless otherwise noted.

N^{α}-Fmoc-Rink amide resin purchased from Novabiochem (100-200 mesh, 0.62 mmol/g loading) was used for SPPS.

MPLC purifications were performed with a Orinedia preparative HPLC (BRIX 2802) on silica gel columns.

RP-HPLC purifications were performed on a Waters preparative HPLC (2767) at room temperature with reverse-phase C18 columns (X Select CSH C18 5 μm, 30 mm×150 mm).

HPLC solvents are H₂O containing 0.05% trifluoroacetic acid (mobile phase A) and acetonitrile (mobile phase B).

HPLC analysis was carried out with a Shimadzu LCMS (2020 series) containing a binary pump (LC-20AD), micro vacuum degasser, auto sampler (SIL-20AC HT), thermostat column compartment (CTO-20A), variable wavelength detector (SPD-M20A). HPLC data was analyzed using Lab Solutions software from the Shimadzu LCMS (2020 series). A Kinetex EVO column (2.6 μm, 100 Å, 4.6×100 mm) was used with a flow rate of 1.0 mL/min.

LCMS analysis was carried out with a Shimadzu LCMS (2020 series) containing a binary pump (LC-20ADXR), micro vacuum degasser, auto sampler (SIL-20AC XR), thermostat column compartment (CTO-20AC), variable wavelength detector (SPD-M20A). LCMS data was analyzed using Lab Solutions software from Agilent Technologies. An Ascentis Express C18 column (2.7 μm, 3.0×50 mm) was used with a flow rate of 1.5 mL/min.

¹H NMR spectra were recorded using an AVANCE III HD 300 MHz, AVANCE NEO 400 MHz, or Bruker 300 MHz or 400 MHz. Chemical shifts are reported in δ (ppm) relative to TMS₄Si (in CDCl₃) as internal standard using Bruker TopSpin software unless otherwise noted.

Examples disclosed herein were prepared according to the procedures outlined above.

### EXAMPLES

### Example 1: Compound 1

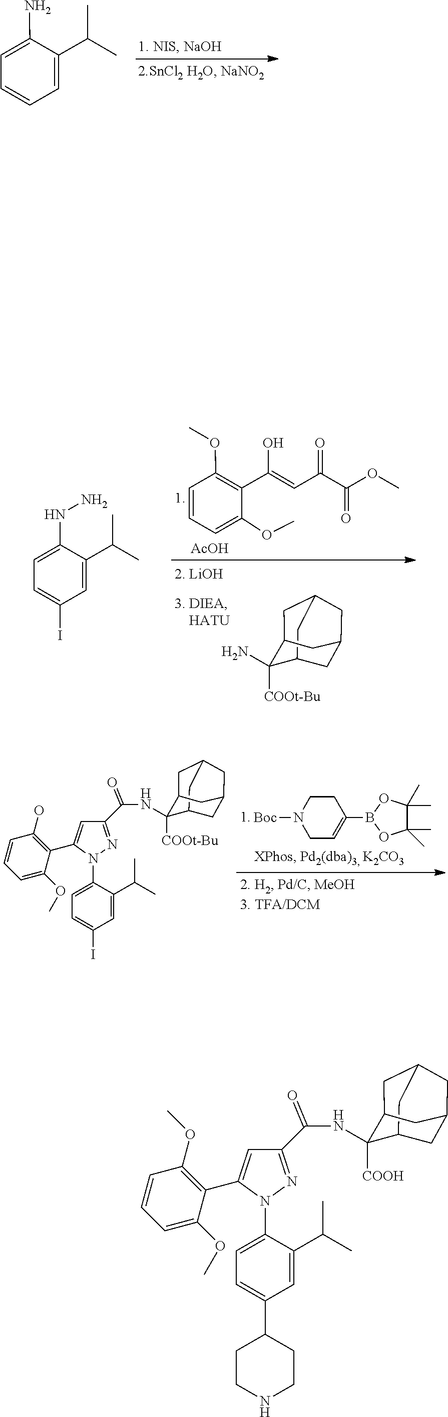

Step 1: Into a 1-L round bottom flask, was placed with a mixture of 2-isopropylaniline (20 g, 1 Eq, 0.15 mol), NaOH (12 g, 2.0 Eq, 0.30 mol), NIS (35 g, 1.1 Eq, 0.16 mol), H₂O (25 mL) and MeOH (500 mL). The reaction mixture was stirred at 25° C. for 16 hours. The mixture was diluted with water (500 mL), extracted with EtOAc (500 mL×3), then the combined organic layers were washed with water (500 mL×2), brine (500 mL), dried over anhydrous Na₂SO₄ and concentrated under reduced pressure. The crude product was purified by MPLC to provide 4-iodo-2-isopropylaniline (23 g, 79 mmol, 54%) as an off-white solid. [M+H]⁺=262.1.

Step 2: Into a 250-mL vial, was placed a mixture of 4-iodo-2-isopropylaniline (5.0 g, 1 Eq, 19 mmol), sodium nitrite (1.3 g, 0.79 mL, 0.98 Eq, 19 mmol), concentrated HCl (50 mL) and H₂O (25 mL). The reaction mixture was stirred at 0° C. for 1 hour, then stannous chloride dihydrate (20 g, 7.4 mL, 4.6 Eq, 89 mmol) and concentrated HCl (50 mL) were added and the reaction mixture was stirred at 25° C. for an additional 2 hours. The mixture was quenched with Et₂O (25 mL), then the reaction mixture was filtered through a pad of celite to afford (4-iodo-2-isopropylphenyl)hydrazine (8.0 g, 14 mmol, 76%) as a light yellow solid, which was used directly for next step without any purification. [M+H]⁺=275.0.

Step 3: Into a 40-mL round bottom flask, was placed a mixture of methyl (Z)-4-(2,6-dimethoxyphenyl)-4-hydroxy-2-oxobut-3-enoate (2.0 g, 50% Wt, 1 Eq, 3.8 mmol), (4-iodo-2-isopropylphenyl)hydrazine (1.0 g, 0.96 Eq, 3.6 mmol) and AcOH (20 mL). The reaction mixture was stirred at 90° C. for 4 hours. The reaction mixture was concentrated under reduced pressure to afford methyl 5-(2,6-dimethoxyphenyl)-1-(4-iodo-2-isopropylphenyl)-1H-pyrazole-3-carboxylate (1.56 g, 1.5 mmol, 41%) as a light yellow solid, which was used directly in the next step without further purification. [M+H]⁺=507.3.

Step 4: Into a 40-mL vial, was placed a mixture of methyl 5-(2,6-dimethoxyphenyl)-1-(4-iodo-2-isopropylphenyl)-1H-pyrazole-3-carboxylate (1.55 g, 1 Eq, 3.06 mmol), lithium hydroxide (700 mg, 9.55 Eq, 29.2 mmol), MeOH (15 mL) and H₂O (2 mL). The reaction mixture was stirred at 25° C. for 16 hours. The reaction mixture was concentrated under reduced pressure to remove most of the MeOH, then the residue was diluted with water (20 mL), and the pH value was adjusted to 6.0 by addition of a saturated NaHSO₄ solution. The aqueous solution was extracted with DCM (30 mL×3), dried over anhydrous Na₂SO₄, and concentrated under reduced pressure to afford 5-(2,6-dimethoxyphenyl)-1-(4-iodo-2-isopropylphenyl)-1H-pyrazole-3-carboxylic acid (500 mg, 0.76 mmol, 25%) as a light-yellow solid, which was used directly in the next step without any purification. [M+H]⁺=493.1.

Step 5: Into a 40-mi vial, was placed a mixture of 5-(2,6-dimethoxyphenyl)-1-(4-iodo-2-isopropylphenyl)-1H-pyrazole-3-carboxylic acid (500 mg, 1 Eq, 1.02 mmol), DIEA (396 mg, 534 μL, 3.02 Eq, 3.06 mmol), HATU (460 mg, 1.19 Eq, 1.21 mmol) and DMF (5 mL). The reaction mixture was stirred at 20° C. for 10 minutes, then tert-butyl (1R,3S,5r,7r)-2-aminoadamantane-2-carboxylate (260 mg, 1.02 Eq, 1.03 mmol) was added and the reaction mixture was stirred at 25° C. for an additional 2 hours. The mixture was directly purified by MPLC to provide tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (510 mg, 0.56 mmol, 55%, 80% Purity) as an off-white solid. [M+H]⁺=726.3.

Step 6: Into a 20-mL vial under an inert atmosphere of nitrogen, was placed a mixture of tert-butyl (1R,3 S,5r,7r)-2-(5-(2,6-dimethoxyphenyl)-1-(4-iodo-2-isopropylphenyl)-1H-pyrazole-3-carboxamido)adamantane-2-carboxylate (150 mg, 1 Eq, 207 μmol), Pd₂(dba)₃ (19 mg, 0.10 Eq, 21 μmol), tert-butyl 4-(4,4,5,5-tetramethyl-1,3,2-dioxaborolan-2-yl)-3,6-dihydropyridine-1(2H)-carboxylate (140 mg, 2.19 Eq, 453 μmol), 2-(dicyclohexylphosphanyl)-2′,4′,6′-tris(isopropyl)biphenyl (10 mg, 0.10 Eq, 21 μmol), K₂CO₃ (70 mug, 2.5 Eq, 0.51 mmol), 1,4-dioxane (4 mL) and H₂O (0.4 mL). The reaction mixture was stirred at 120° C. for 2 hours. The mixture was concentrated and the crude product was purified by MPLC to provide tert-butyl 4-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)-3,6-dihydropyridine-1(2H)-carboxylate (120 mg, 0.12 mmol, 57%) as a yellow oil. [M+H]⁺=781.4.

Step 7: Into a 50-mL round-bottom flask under an inert atmosphere of nitrogen, was placed tert-butyl 4-(4-(3-(((1R,3 S,5r,7r)-2-(tert-butoxycarbonyl)adamantan-2-yl)carbamoyl)-5-(2,6-dimethoxyphenyl)-1H-pyrazol-1-yl)-3-isopropylphenyl)-3,6-dihydropyridine-1(2H)-carboxylate (120 mg, 77% Wt, 1 Eq, 118 μmol) and MeOH (3 mL), to which was carefully added Pd/C (25 μg, 2.0 Eq, 0.23 mmol). The flask was evacuated and flushed with H₂ three times. The mixture was stirred at 25° C. for 4 hours. The reaction mixture was filtered through a pad of celite, then the filtrate was concentrated and purified by MPLC to provide tert-butyl 4-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)piperidine-1-carboxylate (100 mg, 0.11 mmol, 97%) as a white solid. [M+H]⁺=783.4.

Step 8: Into a 8-mL vial, was placed a mixture of tert-butyl 4-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)piperidine-1-carboxylate (30 mg, 1 Eq, 38 μmol) and DCM (1 mL), to which was added TFA (0.2 mL). The reaction mixture was stirred at 25° C. for 1 hour. The mixture was concentrated under reduced pressure and the crude residue was purified by Prep-HPLC to provide (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylic acid-2,2,2-trifluoroacetic acid (1/1) (11.2 mg, 15.1 μmol, 39%) as a white solid. MS: Calc'd for C₃₇H₄₆N₄O₅: 626.34, found [M+H]⁺: 627.3.

### Example 2: Compound 2

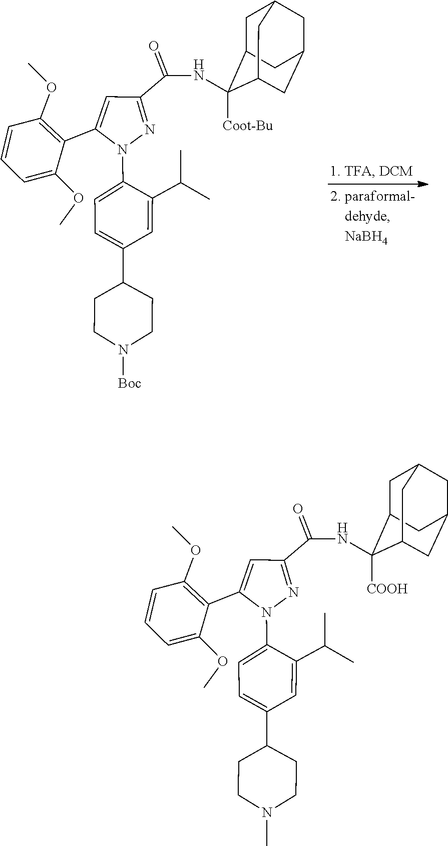

Step 1: Into a 8-mL vial, was placed with a mixture of tert-butyl 4-(4-(3-(((1R,3 S,5r,7r)-2-(tert-butoxycarbonyl)adamantan-2-yl)carbamoyl)-5-(2,6-dimethoxyphenyl)-1H-pyrazol-1-yl)-3-isopropylphenyl)piperidine-1-carboxylate (30 mg, 1 Eq, 38 μmol) and DCM (0.3 mL), to which was added TFA (0.06 mL). The reaction mixture was stirred at 25° C. for 2 hours. The mixture was concentrated under reduced pressure to afford (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylic acid (35 mg, 34 μmol, 87%) as a light yellow oil, which was used directly in the next step without any purification. [M+H]⁺=627.4.

Step 2: Into a 8 mL vial, was placed a mixture of (1R,3 S,5r,7r)-2-(5-(2,6-dimethoxyphenyl)-1-(2-isopropyl-4-(piperidin-4-yl)phenyl)-1H-pyrazole-3-carboxamido)adamantane-2-carboxylic acid (35 mg, 60% Wt, 1 Eq, 34 kmol), paraformaldehyde (5 mg, 5 μL, 5 Eq, 0.2 mmol), sodium tetrahydroborate (5 mg, 5 μL, 4 Eq, 0.1 mmol) and MeOH (0.4 mL). The reaction mixture was stirred at 25° C. for 4 hours then the mixture was concentrated under reduced pressure and the crude product was purified by Prep-HPLC to provide the trifluoroacetic acid salt of (1R,3 S,5r,7r)-2-(5-(2,6-dimethoxyphenyl)-1-(2-isopropyl-4-(1-methylpiperidin-4-yl)phenyl)-1H-pyrazole-3-carboxamido)adamantane-2-carboxylic acid (11.2 mg, 14.7 μmol, 44%) as a white solid. MS: Calc'd for C₃₉H₄N₄O₅: 640.36, found [M+H]⁺: 641.3.

### Example 3: Compound 3

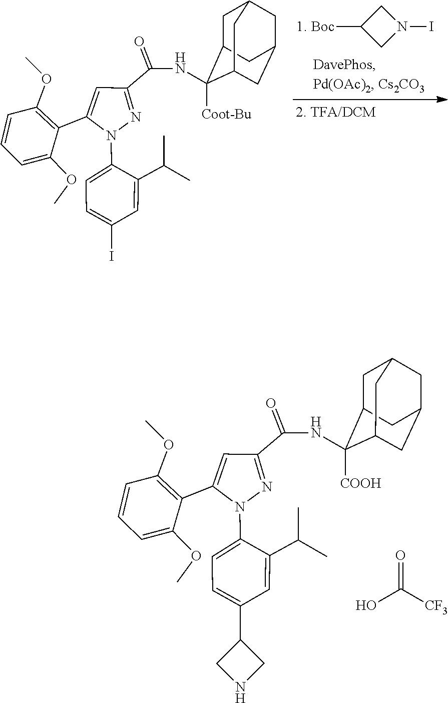

Step 1: Into an 8-mL vial under an inert atmosphere of nitrogen, was placed a mixture of tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (50 mg, 1 Eq, 69 μmol), PdOAc₂ (2 mg, 0.1 Eq, 9 μmol), tert-butyl 3-iodoazetidine-1-carboxylate (29 mg, 1.5 Eq, 0.10 mmol), 2′-dicyclohexylphosphino-2-(N,N-dimethylamino)biphenyl (6 mg, 0.2 Eq, 0.02 mmol), Cs₂CO₃ (67 mg, 3.0 Eq, 0.21 mmol) and ACN (2 mL). The reaction mixture was stirred at 80° C. for 16 hours under N₂ (Blue LED, 450 nm, 100 W). The mixture was directly purified by MPLC to afford tert-butyl 3-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)azetidine-1-carboxylate (10 mg, 13 μmol, 19%) as a yellow oil.

Step 2: The product from Step 1 was treated with TFA in a manner similar to Example 1, Step 8 to provide (1R,3S,5r,7r)-2-(1-(4-(azetidin-3-yl)-2-isopropylphenyl)-5-(2,6-dimethoxyphenyl)-1H-pyrazole-3-carboxamido)adamantane-2-carboxylic acid-2,2,2-trifluoroacetic acid (1/1) (2.2 mg, 3.1 μmol, 23%) as a brown solid. MS: Calc'd for C₃₅H₄₂N₄O₅: 598.31, found [M+H]⁺: 599.3.

### Example 4: Compound 4

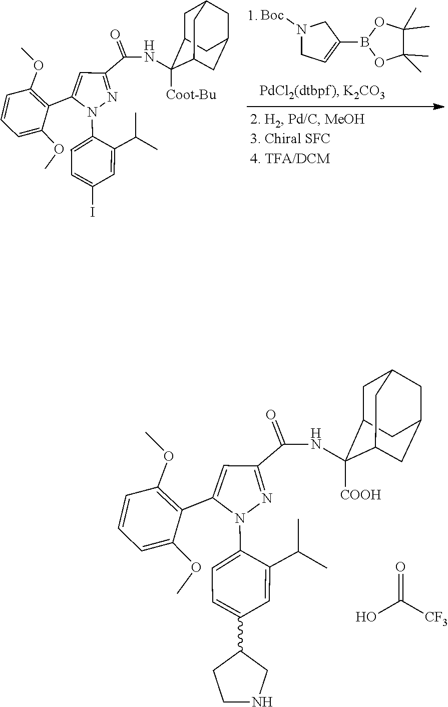

Step 1: Into a 40-mL vial under an inert atmosphere of nitrogen, was placed a mixture of tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (³10 mg, 1 Eq, 427 μmol), 1,1′-bis(di-t-butylphosphino)ferrocene palladium dichloride (28 mg, 0.10 Eq, 43 μmol), tert-butyl 3-(4,4,5,5-tetramethyl-1,3,2-dioxaborolan-2-yl)-2,5-dihydro-1H-pyrrole-1-carboxylate (189 mg, 1.50 Eq, 640 μmol), K₂CO₃ (177 mg, 3.00 Eq, 1.28 mmol), 1,4-dioxane (10 mL) and water (1 mL). The reaction mixture was stirred at 90° C. for 3 hours under N₂. The mixture was directly purified by MPLC to afford tert-butyl 3-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)-2,5-dihydro-1H-pyrrole-1-carboxylate (320 mg, 417 μmol, 97.7%) as a yellow oil.

Step 2: The product from Step 2 is treated with H₂, Pd/C and MeOH in a manner similar to Example 1, Step 7 to provide tert-butyl 3-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)pyrrolidine-1-carboxylate (300 mg, 390 mol, 93.5%) as a white solid.

Step 3: The crude product from Step 2 was purified by Chiral-SFC to provide the first peak as a single isomer of tert-butyl-3-(4-(3-(((1R,3S,5R,7R)-2-(tert-butoxycarbonyl)adamantan-2-yl)carbamoyl)-5-(2,6-dimethoxyphenyl)-1-H-pyrazol-1-yl)-3-isopropylphenyl)pyrrolidine-1-carboxylate (90 mg, 0.12 mmol, 45%) as a white solid.

Step 4: The product from Step 3 was treated with TFA in a manner similar to Example 1, Step 8 to provide a single isomer of (1R,3 S,5R,7R)-2-(5-(2,6-dimethoxyphenyl)-1-(2-isopropyl-4-(-pyrrolidin-3-yl)phenyl)-1H-pyrazole-3-carboxamido)adamantane-2-carboxylic acid-2,2,2-trifluoroacetic acid (1/1) (32.5 mg, 44.7 μmol, 38%) as a white solid. MS: Calc'd for C₃₆H₄₄N₄O₅: 612.33, found [M+H]⁺: 613.4.

### Example 5: Compound 5

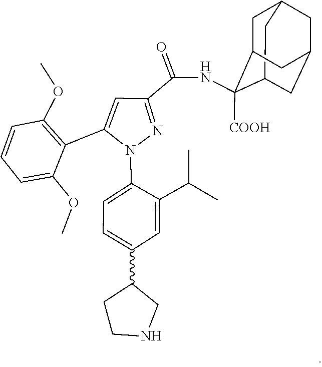 [SMILES: COc1cccc(OC)c1-c1cc(C(=O)NC2(C(=O)O)C3CC4CC(C3)CC2C4)nn1-c1ccc(C2CCNC2)cc1C(C)C]

Step 1: The crude product from Example 4, Step 2 was purified by Chiral-SFC to provide the second peak as the opposite single isomer or tert-butyl-3-(4-(3-(((1R,3S,5S,7S)-2-(tert-butoxycarbonyl)adamantan-2-yl)carbamoyl)-5-(2,6-dimethoxyphenyl)-1H-pyrazol-1-yl)-3-isopropylphenyl)pyrrolidine-1-carboxylate (80 mg, 0.10 mmol, 40%) as a white solid.

Step 2: The product from Step 1 was treated with TFA in a manner similar to Example 1, Step 8 to provide a single isomer (with opposite stereochemistry as compared to Compound 4) of (1R,3S,5S,7S)-2-(5-(2,6-dimethoxyphenyl)-1-(2-isopropyl-4-(-pyrrolidin-3-yl)phenyl)-1H -pyrazole-3-carboxamido)adamantane-2-carboxylic acid-2,2,2-trifluoroacetic acid (1/1) (42.4 mg, 58.3 μmol, 56%) as a white solid. MS: Calc'd for C₃₆H₄₄N₄O₅: 612.33, found [M+H]⁺: 613.4.

### Example 6: Compound 6

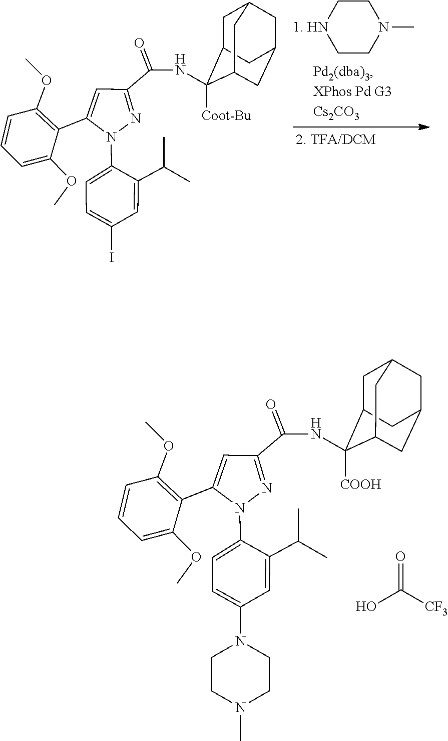

Step 1: Into a 8 mL vial under an inert atmosphere of nitrogen, was placed a mixture of tert-butyl (1R,3 S,5r,7r)-2-(5-(2,6-dimethoxyphenyl)-1-(4-iodo-2-isopropylphenyl)-1H-pyrazole-3-carboxamido)adamantane-2-carboxylate (25 mg, 1 Eq, 34 mol), 1-methylpiperazine (4.1 mg, 1.2 Eq, 41 μmol), Cs₂CO₃ (34 mg, 3.0 Eq, 0.10 mmol), XPhos Pd G3 (5.8 μg, 0.20 Eq, 6.9 μmol), Pd₂(dba)₃ (3.2 mg, 0.10 Eq, 3.5 μmol) and 1,4-dioxane (0.25 mL). The reaction mixture was stirred at 120° C. for 16 hours. The mixture was directly purified by MPLC to afford tert-butyl (1R,3S,5r,7r)-2-(5-(2,6-dimethoxyphenyl)-1-(2-isopropyl-4-(4-methylpiperazin-1-yl)phenyl)-1H-pyrazole-3-carboxamido)adamantane-2-carboxylate (18 mg, 26 μmol, 75%) as a light-yellow solid.

Step 2: The product from Step 1 was treated with TFA in a manner similar to Example 1, Step 8 to provide (1R,3S,5r,7r)-2-(5-(2,6-dimethoxyphenyl)-1-(2-isopropyl-4-(4-methylpiperazin-1-yl)phenyl)-1H-pyrazole-3-carboxamido)adamantane-2-carboxylic acid-2,2,2-trifluoroacetic acid (1/1) (12.0 mg, 15.9 μmol, 61%) as a white solid. MS: Calc'd for C₃₇H₄₇NO₅: 641.35, found [M+H]⁺: 642.3.

### Example 7: Compound 7

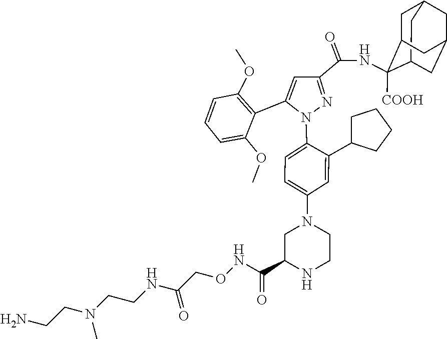 [SMILES: COc1cccc(OC)c1-c1cc(C(=O)NC2(C(=O)O)C3CC4CC(C3)CC2C4)nn1-c1ccc(N2CCN[C@@H](C(=O)NOCC(=O)NCCN(C)CCN)C2)cc1C1CCCC1]

### Example 8: Compound 8

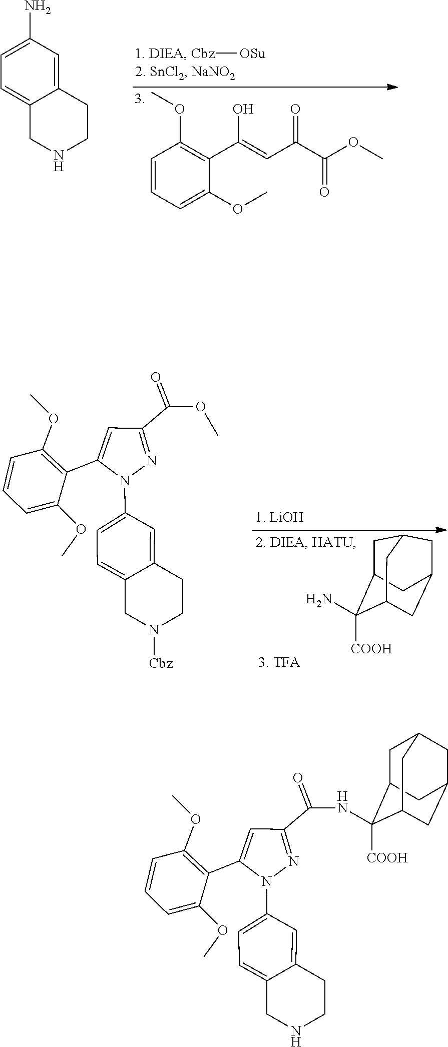

Step 1: Into the 40-mL vial, was placed a mixture of 1,2,3,4-tetrahydroisoquinolin-6-amine (600 mg, 1 Eq, 4.05 mmol), DIEA (1.57 g, 2.12 mL, 3.00 Eq, 12.1 mmol), benzyl (2,5-dioxopyrrolidin-1-yl) carbonate (1.51 g, 1.50 Eq, 6.06 mmol), and DMF (10 mL). The reaction mixture was stirred at 25° C. for 1 hour. The crude product was purified by Prep-HPLC to provide benzyl 6-amino-3,4-dihydroisoquinoline-2(1H)-carboxylate (800 mg, 2.83 mmol, 70.0%) as a white solid. [M+H]283.1.

Step 2: Into a 50-mL three-necked round bottom flask under an inert atmosphere of nitrogen, was placed a mixture of benzyl 6-amino-3,4-dihy droisoquinoline-2(1H)-carboxylate (800 mg, 1 Eq, 2.83 mmol), HCl (20 mL) and H₂O (10 mL) at 0° C., to which was added a solution of sodium nitrite (160 μL 1.3 Eq, 3.68 mmol) in H₂O (25 mL) dropwise over 2 min. The reaction mixture was stirred at 0° C. for 2 hours, then a solution of dichlorostannane (2.15 g, 557 μL, 4.0 Eq, 11.3 mmol) in HCl (40 mL) was added at 0° C. over 2 min. The reaction mixture was stirred at 25° C. for 2 hours. The reaction mixture was filtered, washed with Et₂O and concentrated to afford benzyl 6-hydrazineyl-3,4-dihydroisoquinoline-2(1H)-carboxylate (700 mg, 2.35 mmol, 83.1%) as a yellow solid, which was used directly for next step without further purification. [M+H−17]⁺281.0.

Step 3: The product from Step 2 was treated with methyl (Z)-4-(2,6-dimethoxyphenyl)-4-hydroxy-2-oxobut-3-enoate (627 mg, 1.00 Eq, 2.35 mmol) and AcOH (15 mL) in a manner similar to Example 1, Step 3 to provide benzyl 6-(5-(2,6-dimethoxyphenyl)-3-(methoxycarbonyl)-1H-pyrazol-1-yl)-3,4-dihydroisoquinoline-2(1H)-carboxylate (600 mg, 1.14 mmol, 48.3%) as a yellow oil.

Step 4: The product from Step 3 was treated with LiOH in a manner similar to Example 1, Step 4 to provide 1-(2-((benzyloxy)carbonyl)-1,2,3,4-tetrahydroisoquinolin-6-yl)-5-(2,6-dimethoxyphenyl)-1H-pyrazole-3-carboxylic acid (240 mg, 467 μmol, 82.2%) as a light yellow solid, which was used directly in the next step without any purification. [M+H]⁺=514.3.

Step 5: The product from Step 4 was treated with HATU (163 mg, 1.10 Eq, 429 μmol), DIEA (151 mg, 204 μL, 3.00 Eq, 1.17 mmol) and DMF (5 mL) in a manner similar to Example 1, Step 5 to provide benzyl 6-(3-(((1R,3S,5r,7r)-2-(tert-butoxycarbonyl)adamantan-2-yl)carbamoyl)-5-(2,6-dimethoxyphenyl)-1H-pyrazol-1-yl)-3,4-dihydroisoquinoline-2(1H)-carboxylate (90 mg, 0.12 mmol, 31%) as a white solid. [M+H]⁺=747.5.

Step 6: The product from Step 5 was treated with TFA in a manner similar to Example 1, Step 8 to provide (1R,3S,5r,7r)-2-(5-(2,6-dimethoxyphenyl)-1-(1,2,3,4-tetrahydroisoquinolin-6-yl)-1H-pyrazole-3-carboxamido)adamantane-2-carboxylic acid-2,2,2-trifluoroacetic acid (1/1) (11.5 mg, 17.1 μmol, 64%) as a white solid. MS: Calc'd for C₃₂H₃(N₄O₅: 556.27, found [M+H]⁺: 557.2.

### Example 9: Compound 9

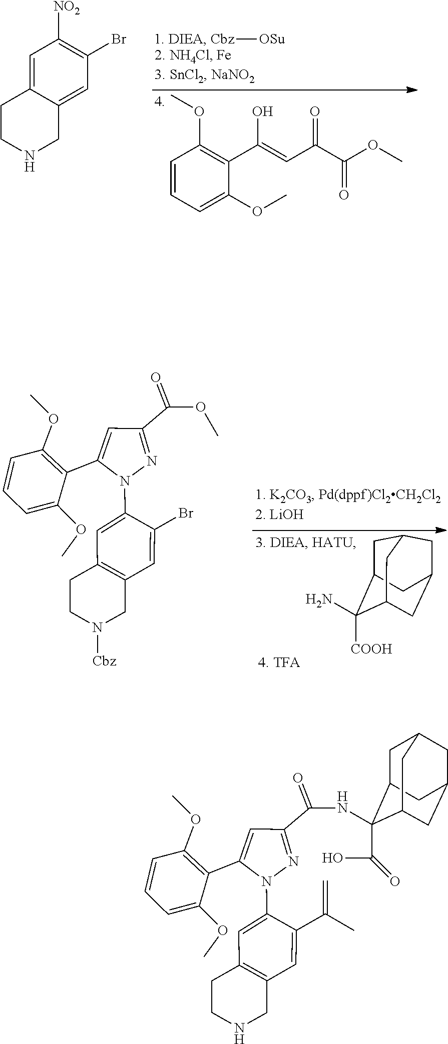

Step 1: 7-bromo-6-nitro-1,2,3,4-tetrahydroisoquinoline (1.0 g, 1 Eq, 3.9 mmol) was treated with DIEA (1.5 g, 2.0 mL, 3.0 Eq, 12 mmol) and benzyl (2,5-dioxopyrrolidin-1-yl) carbonate (1.5 g, 1.5 Eq, 6.0 mmol), and DMF (15 mL) in a manner similar to Example 8, Step 1 to provide benzyl 7-bromo-6-nitro-3,4-dihydroisoquinoline-2(1H)-carboxylate (1.3 g, 3.3 mmol, 85%) as a white solid. [M+H]⁺=391.0.

Step 2: Into a 40-mL vial, was placed with a mixture of benzyl 7-bromo-6-nitro-3,4-dihydroisoquinoline-2(1H)-carboxylate (1.2 g, 1 Eq, 3.1 mmol), ammonium chloride (0.82 g, 0.56 mL, 5.0 Eq, 15 mmol), iron (0.51 g, 65 μL, 3.0 Eq, 9.1 mmol), EtOH (15 mL), and H₂O (5 mL). The reaction mixture was stirred at 70° C. for 2 hours. The mixture was diluted with water (100 mL), extracted with EtOAc (50 mL×3), the combined organic layers were washed with water (50 mL×2), brine (50 mL), dried over anhydrous Na₂SO₄ and concentrated under reduced pressure. The crude product was purified by MPLC to provide benzyl 6-amino-7-bromo-3,4-dihydroisoquinoline-2(1H)-carboxylate (1.05 g, 2.91 mmol, 95%) as a yellow oil. [M+H]⁺=361.0.

Step 3: The product from Step 2 was treated with sodium nitrite (802 mg, 504 μL, 4.00 Eq, 11.6 mmol) and dichlorostannane (716 mg, 186 μL, 1.30 Eq, 3.78 mmol) in a manner similar to Example 8, Step 2 to afford benzyl 7-bromo-6-hydrazineyl-3,4-dihydroisoquinoline-2(1H)-carboxylate (920 mg, 2.45 mmol, 84.1%) as a yellow solid, which was used directly in the next step without any purification. [M+H-17]⁺=361.0.

Step 4: The product from Step 3 was treated with methyl (Z)-4-(2,6-dimethoxyphenyl)₄-hydroxy-2-oxobut-3-enoate (650 mg, 0.998 Eq, 2.44 mmol) and AcOH (20 mL) in a manner similar to Example 8, Step 3 to provide benzyl 7-bromo-6-(5-(2,6-dimethoxyphenyl)-3-(methoxycarbonyl)-1H-pyrazol-1-yl)-3,4-dihydroisoquinoline-2(1H)-carboxylate (560 mg, 923 μmol, 37.8%) as a yellow oil. [M+H]⁺=606.1.

Step 5: Into a 40-mL vial, was placed a mixture of benzyl 7-bromo-6-(5-(2,6-dimethoxyphenyl)-3-(methoxycarbonyl)-1H-pyrazol-1-yl)-3,4-dihydroisoquinoline-2(1H)-carboxylate (560 mg, 1 Eq, 923 μmol), 4,4,5,5-tetramethyl-2-(prop-1-en-2-yl)-1,3,2-dioxaborolane (310 mg, 2.00 Eq, 1.84 mmol), 1,1′-bis(diphenylphosphino)ferrocene-palladium(II) dichloride (68 mg, 0.10 Eq, 93 μmol) and K₂CO₃ (383 mg, 3.00 Eq, 2.77 mmol) in 1,4-dioxane (10 mL), and H₂O (1 mL). The reaction mixture was stirred at 100° C. for 2 hours. The reaction mixture was concentrated under reduced pressure to remove dioxane, extracted with DCM (50 mL×3), then the combined organic layers were dried over anhydrous Na₂SO₄, and concentrated. The crude product was purified by MPLC to afford benzyl 6-(5-(2,6-dimethoxyphenyl)-3-(methoxycarbonyl)-1H-pyrazol-1-yl)-7-(prop-1-en-2-yl)-3,4-dihydroisoquinoline-2(1H)-carboxylate (460 μg, 810 μmol, 87.8%) as an off-white solid. [M+H]⁺=568.2.

Step 6: The product from Step 5 was treated with LiOH (59 mg, 3.0 Eq, 2.5 mmol) in a manner similar to Example 8, Step 4 to provide 1-(2-((benzyloxy)carbonyl)-7-(prop-1-en-2-yl)-1,2,3,4-tetrahydroisoquinolin-6-yl)-5-(2,6-dimethoxyphenyl)-1H-pyrazole-3-carboxylic acid (420 mg, 759 μmol, 93.6%) as a light yellow solid, which was used directly in the next step without any purification. [M+H]⁺=554.2.

Step 7: The product from Step 6 was treated with HATU (330 mg, 1.20 Eq, 868 μmol), DIEA (280 mg, 377 μL, 3.00 Eq, 2.17 mmol) and tert-butyl (1R,3S,5r,7r)-2-aminoadamantane-2-carboxylate (218 mg, 1.20 Eq, 867 μmol) in a manner similar to Example 8, Step 5 to provide benzyl 6-(3-(((1R,3S,5r,7r)-2-(tert-butoxycarbonyl)adamantan-2-yl)carbamoyl)-5-(2,6-dimethoxyphenyl)-1H-pyrazol-1-yl)-7-(prop-1-en-2-yl)-3,4-dihydroisoquinoline-2(1H)-carboxylate (410 mg, 521 μmol, 72.1%) as a white solid. [M+H]⁺=787.5.

Step 8: The product from Step 7 was treated with TFA in a manner similar to Example 8, Step 6 to provide (1R,3S,5r,7r)-2-(5-(2,6-dimethoxyphenyl)-1-(7-(prop-1-en-2-yl)-1,2,3,4-tetrahydroisoquinolin-6-yl)-1H-pyrazole-3-carboxamido)adamantane-2-carboxylic acid-2,2,2-trifluoroacetic acid (1/1) (11.4 mg, 16.0 μmol, 25%) as a white solid. MS: Calc'd for C₃₅H₄₀N₄O₅: 596.3, found [M+H]⁺: 597.3.

### Example 10: Compound 10

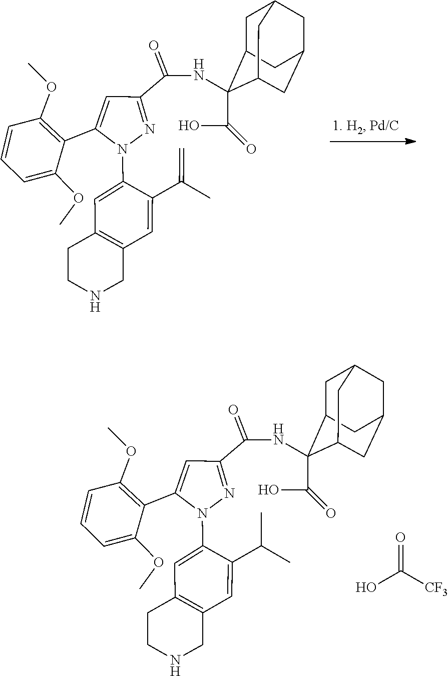 [SMILES: C=C(C)c1cc2c(cc1-n1nc(C(=O)NC3(C(=O)O)C4CC5CC(C4)CC3C5)cc1-c1c(OC)cccc1OC)CCNC2 ; COc1cccc(OC)c1-c1cc(C(=O)NC2(C(=O)O)C3CC4CC(C3)CC2C4)nn1-c1cc2c(cc1C(C)C)CNCC2 ; O=C(O)C(F)(F)F]

(1R,3S,5r,7r)-2-(5-(2,6-dimethoxyphenyl)-1-(7-(prop-1-en-2-yl)-1,2,3,4-tetrahydroisoquinolin-6-yl)-1H-pyrazole-3-carboxamido)adamantane-2-carboxylic acid-2,2,2-trifluoroacetic acid (1/1) (Compound 9, 20 mg, 1 Eq, 34 μmol) was treated with H₂ and Pd/C (29 mg, 8.1 Eq, 0.27 mmol) in a manner similar to Example 1, Step 7 to provide the 2,2,2-trifluoroacetic acid of (1R,3S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylic acid (10.3 mg, 14.5 μmol, 43%) as a white solid. MS: Calc'd for C₃₅H₁₂N₄O₅: 598.32, found [M+H]⁺: 599.4.

### Example 11: Compound 11

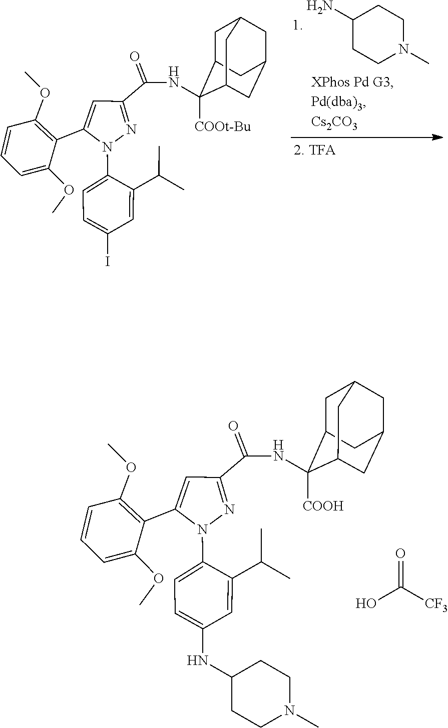

Step 1: tert-Butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (25 mg, 1 Eq, 34 μmol) was treated with 1-methylpiperidin-4-amine (5 μg, 1 Eq, 0.04 mmol), Cs₂CO₃ (34 μg, 3.0 Eq, 0.10 mmol), Pd₂(dba)₃ (4 mg, 0.1 Eq, 4 μmol), and XPhos Pd G3 (5.9 mg, 0.20 Eq, 7.0 μmol) in a manner similar to Example 6, step 1 to provide tert-butyl (1R,3S,5r,7r)-2-(5-(2,6-dimethoxyphenyl)-1-(2-isopropyl-4-((1-methylpiperidin-4-yl)amino)phenyl)-1H-pyrazole-3-carboxamido)adamantane-2-carboxylate (16 mg, 22 μmol, 65%) as a light-yellow solid.

The product from Step 1 was treated with TFA in a manner similar to Example 1, Step 8 to provide the 2,2,2-trifluoroacetic acid (1/1) salt of (1R,3 S,5r,7r)-2-(5-(2,6-dimethoxyphenyl)-1-(2-isopropyl-4-((1-methylpiperidin-4-yl)amino)phenyl)-1H-pyrazole-3-carboxamido)adamantane-2-carboxylic acid (10.3 mg, 13.4 μmol, 60%) as a white solid. MS: Calc'd for C₃₈H₄₉N₅O₅ 655.37, found [M+H]⁺: 656.5.

### Example 12: Compound 12

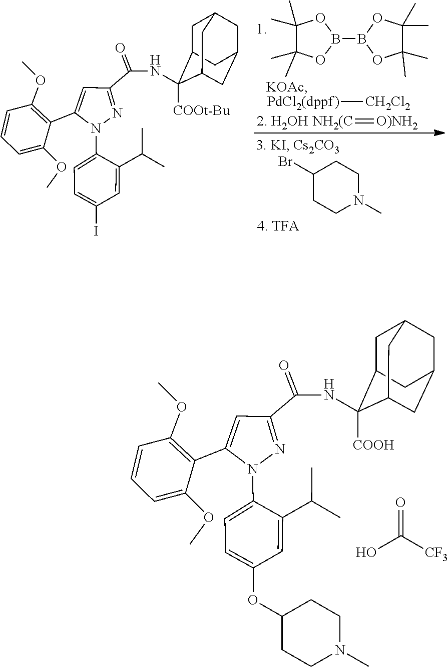

Step 1: Into a 8-mL vial, was placed a mixture of tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (200 mg, 1 Eq, 276 μmol), 4,4,4′,4′,5,5,5′,5′-octamethyl-2,2′-bi(1,3,2-dioxaborolane) (140 mg, 2.00 Eq, 551 μmol), potassium acetate (82 mg, 52 μL, 3.0 Eq, 0.84 mmol) and PdCl₂(dppf)-CH₂Cl₂ (23 mg, 0.10 Eq, 28 μmol) in dioxane (2 mL). The reaction mixture was stirred at 80° C. for 16 hours. The reaction mixture was concentrated under reduced pressure to remove dioxane, extracted with DCM (50 mL×3), dried over anhydrous Na₂SO₄, concentrated and the crude product was purified by MPLC to afford tert-butyl (1R,3S,5r,7r)-2-(5-(2,6-dimethoxyphenyl)-1-(2-isopropyl-4-(4,4,5,5-tetramethyl-1,3,2-dioxaborolan-2-yl)phenyl)-1-pyrazole-3-carboxamido)adamantane-2-carboxylate (180 mg, 248 μmol) as an off-white solid. [M+H]⁺=726.4.

Step 2: Into an 8-mL vial, was placed a mixture of tert-butyl (1R,3S,5r,7r)-2-(5-(2,6-dimethoxyphenyl)-1-(2-isopropyl-4-(4,4,5,5-tetramethyl-1,3,2-dioxaborolan-2-yl)phenyl)-1H-pyrazole-3-carboxamido)adamantane-2-carboxylate (180 mg, 1 Eq, 248 μmol), carbamide peroxide (70 mg, 3.0 Eq, 0.74 mmol), potassium acetate (82 mg, 52 μL, 3.0 Eq, 0.84 mmol) in MeCN (1 mL) and MeOH (1 mL). The reaction mixture was stirred at 25° C. for 1 hour. The reaction mixture was extracted with ethyl acetate (20 mL×3), dried over anhydrous Na₂SO₄, concentrated and the crude product was purified by MPLC to afford tert-butyl (1R,3S,5r,7r)-2-(5-(2,6-dimethoxyphenyl)-1-(4-hydroxy-2-isopropylphenyl)-1H-pyrazole-3-carboxamido)adamantane-2-carboxylate (80 mg, 0.13 mmol, 52%) as an off-white solid. [M+H]⁺=616.3.

Step 3: Into an 80-mL vial, was placed a mixture of tert-butyl (1R,3S,5r,7r)-2-(5-(2,6-dimethoxyphenyl)-1-(4-hydroxy-2-isopropylphenyl)-1H-pyrazole-3-carboxamido)adamantane-2-carboxylate (40 mg, 1 Eq, 65 μmol), 4-bromo-1-methylpiperidine (23 mg, 2.0 Eq, 0.13 mmol), Cs₂CO₃ (63 mg, 3.0 Eq, 0.19 mmol) and KI (11 mg, 1.0 Eq, 66 μmol) in DMF (0.5 mL). The reaction mixture was stirred at 90° C. for 2 hours. The mixture was directly purified by MPLC to afford tert-butyl (1R,3S,5r,7r)-2-(5-(2,6-dimethoxyphenyl)-1-(2-isopropyl-4-((1-methylpiperidin-4-yl)oxy)phenyl)-1H-pyrazole-3-carboxamido)ad amantane-2-carboxylate (20 mg, 28 μmol, 43%) as an off-white solid. [M+H]⁺=713.4.

Step 4: The product from Step 3 was treated with TFA in a manner similar to Example 1, Step 8 to provide the 2,2,2-trifluoroacetic acid (1/1) salt of (1R,3S,5r,7r)-2-(5-(2,6-dimethoxyphenyl)-1-(2-isopropyl-4-((1-methylpiperidin-4-yl)oxy)phenyl)-1H-pyrazole-3-carboxamido)adamantane-2-carboxylic acid (16.1 mg, 20.9 μmol, 74%) as a white solid. MS: Calc'd for C₃₈H₄₈N₄O₆: 656.36, found [M+H]⁺: 657.3.

### Example 13: Compound 13

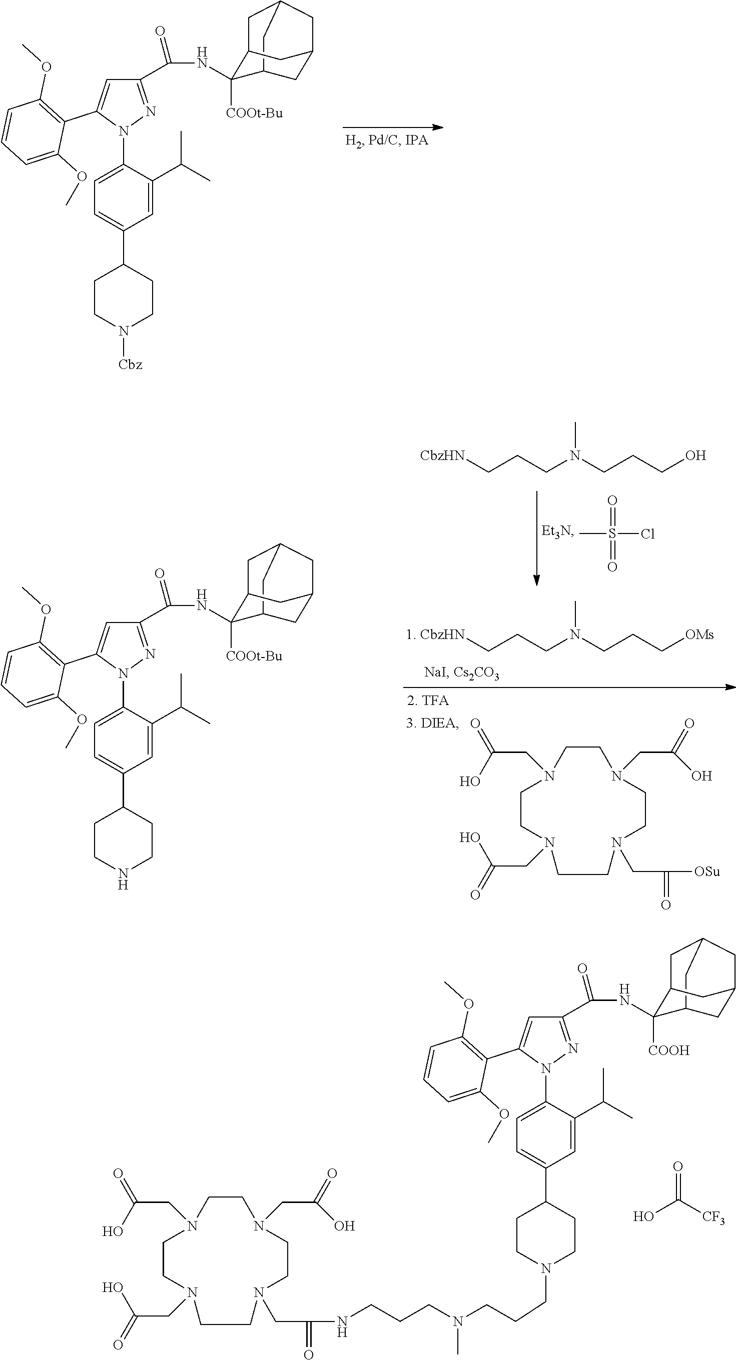

Step 1: benzyl 4-(4-(3-(((1R,3 S,5r,7r)-2-(tert-butoxycarbonyl)adamantan-2-yl)carbamoyl)-5-(2,6-dimethoxyphenyl)-1H-pyrazol-1-yl)-3-isopropylphenyl)-3,6-dihydropyridine-1(2H)-carboxylate (synthesized in a manner similar to Example 1, Steps 1-6) was treated with H₂ and Pd/C (600 mg, 10% Wt, 0.38 Eq, 564 μmol) in a manner similar to Example 1, Step 7 to provide the 2,2,2-trifluoroacetic acid of tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (900 mg, 1.32 mmol, 90%) as a white solid. [M+H]=683.8.

Step 2: Into a 40-mL vial, was placed a mixture of benzyl (3-((3-hydroxypropyl)(methyl)amino)propyl)carbamate (300 mg, 1 Eq, 1.07 mmol), TEA (325 mg, 448 μL, 3.00 Eq, 3.21 mmol) and DCM (5 mL), to which was added methanesulfonyl chloride (184 mg, 124 μL, 1.50 Eq, 1.61 mmol) dropwise at 0° C. The reaction mixture was stirred at 25° C. for 1 hour. The mixture was quenched with water (20 mL), extracted with DCM (20 mL×2), the combined organic layers were washed with brine (20 mL), dried over anhydrous Na₂SO₄, concentrated under reduced pressure to afford 3-((3-(((benzyloxy)carbonyl)amino)propyl)(methyl)amino)propyl methanesulfonate (400 mg, 0.89 mmol, 83%) as a light yellow oil, which was used directly for next step without any purification. [M+H]⁺=359.1.

Step 3: Into an 8-mL vial, was placed a mixture of tert-butyl (1R,3 S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (80 mg, 1 Eq, 0.12 mmol), 3-((3-(((benzyloxy)carbonyl)amino)propyl)(methyl)amino)propyl methanesulfonate (84 mg, 2.0 Eq, 0.23 mmol), sodium iodide (18 mg, 4.9 μL, 1.0 Eq, 0.12 mmol), Cs₂CO₃ (76 mg, 2.0 Eq, 0.23 mmol) and DMSO (2 mL). The reaction mixture was stirred at 100° C. for 2 hours. The mixture was directly purified by MPLC to provide tert-butyl (1R,3S,5r,7r)-2-(1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxamido)adamantane-2-carboxylate (50 mg, 53 μmol, 45%) as a yellow oil. [M+H]⁺=945.7.

Step 4: Into an 8-mL vial, was placed a mixture of tert-butyl (1R,3S,5r,7r)-2-(1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxamido)adamantane-2-carboxylate (50 mg, 1 Eq, 53 μmol) and TFA (2 mL). The reaction mixture was stirred at 60° C. for 2 hours. The mixture was concentrated under reduced pressure to provide (1R,3 S,5r,7r)-2-(1-(4-(1-(3-((3-aminopropyl)(methyl)amino)propyl)piperidin-4-yl)-2-isopropylphenyl)-5-(2,6-dimethoxyphenyl)-1H-pyrazole-3-carboxamido)adamantane-2-carboxylic acid (40 mg, 53 μmol, 100%) as a yellow oil, which was used directly in the next step without any purification. [M+H]⁺=755.6.

Step 5: Into an 8-mL vial, was placed a mixture of (1R,3S,5r,7r)-2-(1-(4-(1-(3-((3-aminopropyl)(methyl)amino)propyl)piperidin-4-yl)-2-isopropylphenyl)-5-(2,6-dimethoxyphenyl)-1H-pyrazole-3-carboxamido)adamantane-2-carboxylic acid (40 mg, 1 Eq, 53 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (80 mg, 3.0 Eq, 0.16 mmol) and DMF (2 mL), to which was added DIEA (34 mg, 46 μL, 5.0 Eq, 0.26 mmol). The reaction mixture was stirred at 25° C. for 1 hour. The crude product was purified by Prep-HPLC to provide the 2,2,2-trifluoroacetic acid salt of 2,2′,2″-(10-(2-((3-((3-(4-(4-(3-(((1R,3S,5r,7r)-2-carboxy adamantan-2-yl)carbamoyl)-5-(2,6-dimethoxyphenyl)-1H-pyrazol-1-yl)-3-isopropylphenyl)piperidin-1-yl)propyl)(methyl)amino)propyl)amino)-2-oxoethyl)-1,4,7,10-tetraazacyclododecane-1,4,7-triyl)triacetic acid (39.4 mg, 31.4 μmol, 59%) as a white solid. MS: Calc'd for C₆₀H₈₈N₁₀O₁₂: 1140.66, found [M+H]⁺: 1141.5.

### Example 14: Compound 14

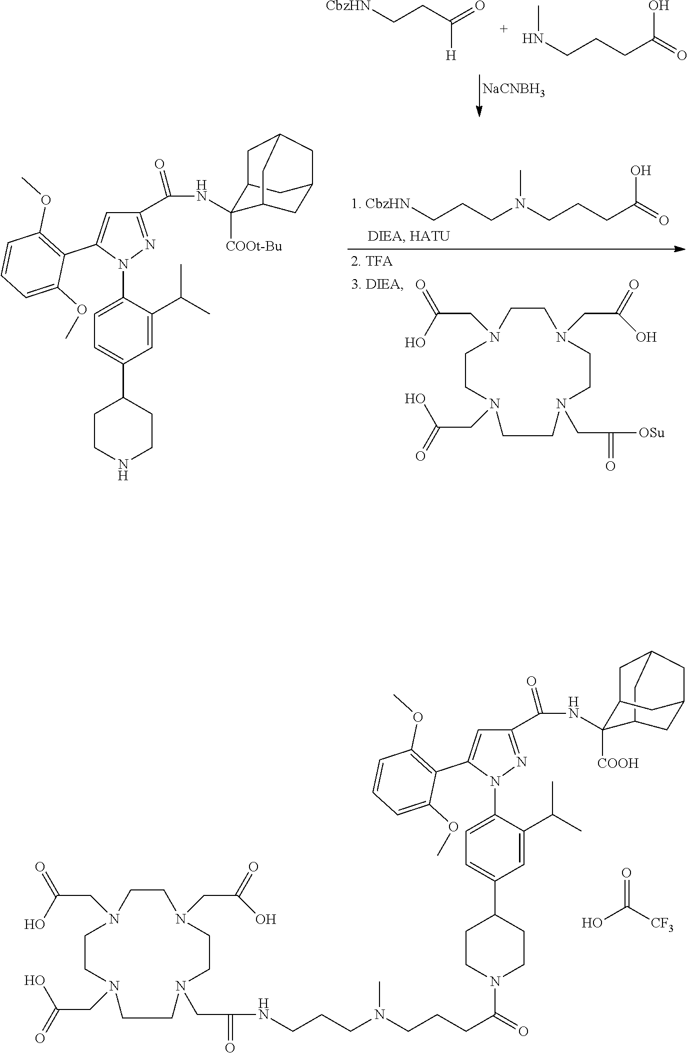

Step 1: Into an 8-mL vial, was placed a mixture of benzyl (3-oxopropyl)carbamate (500 mg, 1 Eq, 2.41 mmol), 4-(methylamino)butanoic acid (339 mg, 1.20 Eq, 2.89 mmol) and MeOH (7 mL). The reaction mixture was stirred at 25° C. for 10 min., then sodium cyanoborohydride (303 mg, 281 μL, 2.00 Eq, 4.82 mmol) was added and the reaction mixture was stirred at 25° C. for an additional 1 hour. The mixture was directly purified by MPLC to afford 4-((3-(((benzyloxy)carbonyl)amino)propyl)(methyl)amino)butanoic acid (450 mg, 1.46 mmol, 60.5%) as a light yellow solid. [M+H]⁺=309.1.

Step 2: Into an 8-mL vial, was placed a mixture of 4-((3-(((benzyloxy)carbonyl)amino)propyl)(methyl)amino)butanoic acid (43 mg, 1.2 Eq, 0.14 mmol), HATU (53 mg, 1.2 Eq, 0.14 mmol), DIEA (45 mug, 61 μL, 3.0 Eq, 0.35 mmol) and DMF (2 mL). The reaction mixture was stirred at 20° C. for 10 minutes, then tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (80 mg, 1 Eq, 0.12 mmol) was added and the reaction mixture was stirred at 25° C. for an additional 1 hour. The mixture was directly purified by MPLC to provide tert-butyl (1R,3S,5r,7r)-2-(1-(4-(1-(4-((3-(((benzyloxy)carbonyl)amino)propyl)(methyl)amino)butanoyl)piperidin-4-yl)-2-isopropylphenyl)-5-(2,6-dimethoxyphenyl)-1H-pyrazole-3-carboxamido)adamantane-2-carboxylate (70 mg, 72 μmol, 61%) as a yellow oil. [M+H]⁺=973.7.

Steps 3 and 4: tert-Butyl (1R,3S,5r,7r)-2-(1-(4-(1-(4-((3-(((benzyloxy)carbonyl)amino)propyl)(methyl)amino)butanoyl)piperidin-4-yl)-2-isopropylphenyl)-5-(2,6-dimethoxyphenyl)-1H-pyrazole-3-carboxamido)adamantane-2-carboxylate was treated with TFA, then 2,2′,2′-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2,2′-(10-(2-((3-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-4-oxobutyl)(methyl)amino)propyl)amino)-2-oxoethyl)-1,4,7,10-tetraazacyclododecane-1,4,7-triyl)triacetic acid (62.7 mug, 48.9 μmol, 77%) as a white solid. MS: Calc'd for C₆₁H₈₈N₁₀O₁₃: 1168.65, found [M+H]⁺: 1169.5.

### Example 15: Compound 15

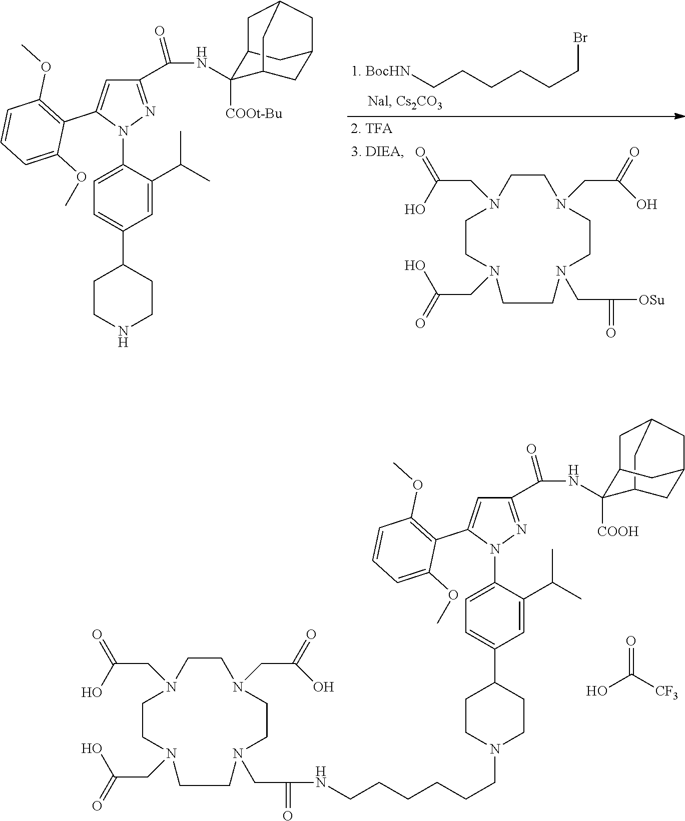

Step 1: tert-Butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (80 mg, 1 Eq, 0.12 mmol) was treated with tert-butyl (6-bromohexyl)carbamate (49 mg, 1.5 Eq, 0.17 mmol), sodium iodide (18 mg, 4.9 μL, 1.0 Eq, 0.12 mmol), and Cs₂CO₃ (76 mg, 2.0 Eq, 0.23 mmol) in a manner similar to Example 13, Step 3 to provide tert-butyl (1R,3S,5r,7r)-2-(1-(4-(1-(6-((tert-butoxycarbonyl)amino)hexyl)piperidin-4-yl)-2-isopropylphenyl)-5-(2,6-dimethoxyphenyl)-1H-pyrazole-3-carboxamido)adamantane-2-carboxylate (70 mg, 79 mol, 68%) as a yellow oil. [M+H]⁺=883.1.

Steps 2 and 3: tert-Butyl (1R,3S,5r,7r)-2-(1-(4-(1-(6-((tert-butoxycarbonyl)amino)hexyl)piperidin-4-yl)-2-isopropylphenyl)-5-(2,6-dimethoxyphenyl)-1H -pyrazole-3-carboxamido)adamantane-2-carboxylate was treated with TFA, then 2,2′,2″(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((6-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)hexyl)amino)-2-oxoethyl)-1,4,7,10-tetraazacyclododecane-1,4,7-triyl)triacetic acid (61.9 mg, 50.5 μmol, 63%) as a white solid. MS: Calc'd for C₅₉H₅N₉O₁₂: 1111.63, found [M+H]⁺: 1112.6.

### Synthesis of Compound 15-Lu

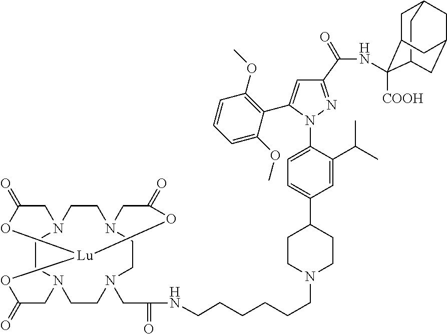

A mixture of 2,2′,2″-(10-(2-((6-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)hexyl)amino)-2-oxoethyl)-1,4,7,10-tetraazacyclododecane-1,4,7-triyl)triacetic acid (25 mg, 1 Eq, 22 μmol), Lutetium (111) chloride (19 mg, 4.8 μL, 3.0 Eq, 68 μmol) and NaOAc/AcOH Buffer (1 mL) was stirred at 80° C. for 1 hour. The crude product was purified by Prep-HPLC to afford the product (13.7 mg, 9.80 μmol, 44%) as a white solid. MS: Calc'd for C₅₉H₈₂LuN₉O₁₂: 1283.55, found [M+H]⁺: 1284.3.

### Synthesis of Compound 15-Ga

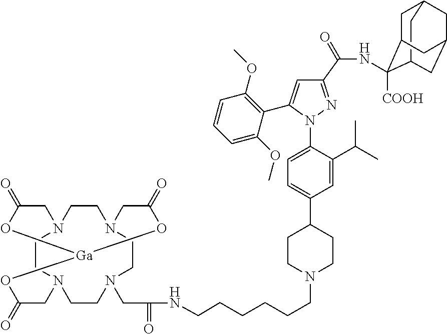

A mixture of 2,2′,2″-(10-(2-((6-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)hexyl)amino)-2-oxoethyl)-1,4,7,10-tetraazacyclododecane-1,4,7-triyl)triacetic acid (25 mg, 1 Eq, 22 μmol), gallium(III) chloride (16 mg, 4.0 Eq, 91 μmol) and NaOAc Buffer (2 mL) was stirred at 80° C. for 30 min. The crude mixture was directly purified by Prep-HPLC to afford the product (18.0 mg, 13.9 mol, 62%) as a white solid. MS: Calc'd for C₉H₈GaN₉O₁₂: 1177.53, found [M+H]⁺: 1178.7.

### Example 16: Compound 16

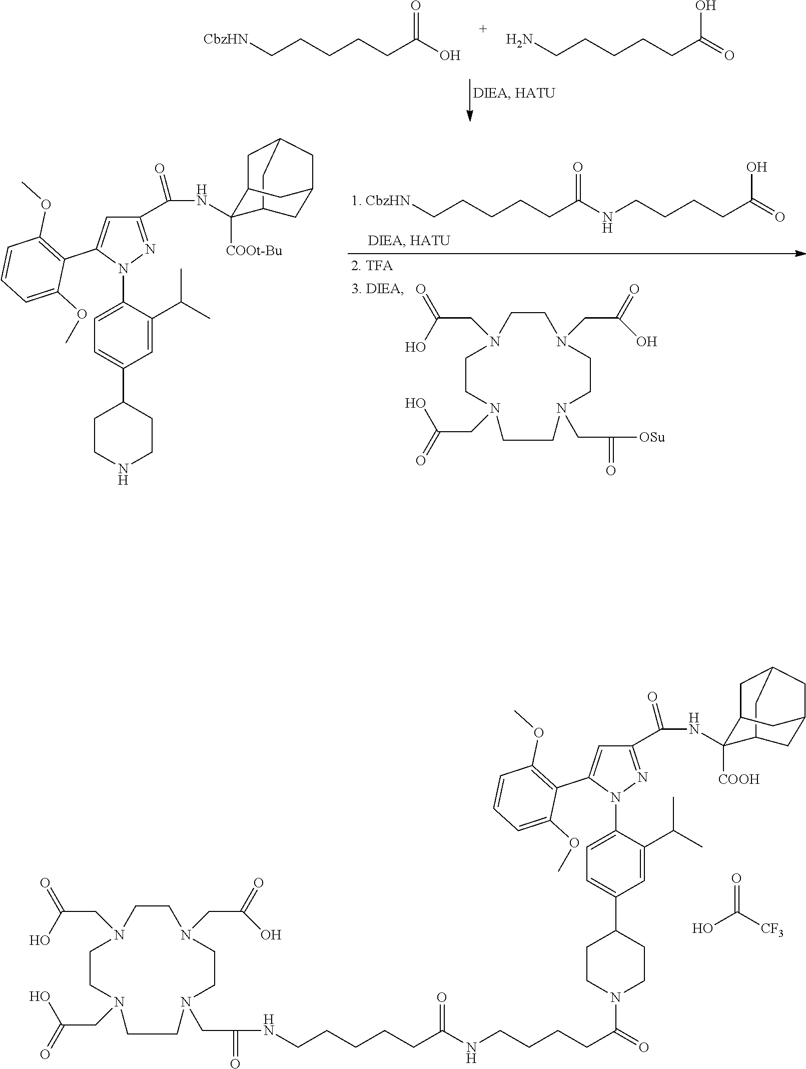

Step 1: Into a 40-mL vial, was placed a mixture of 6-(((benzyloxy)carbonyl)amino)hexanoic acid (500 mg, 1 Eq, 1.88 mmol), HATU (860 mg, 1.20 Eq, 2.26 mmol), DIEA (731 mg, 985 μL, 3.00 Eq, 5.66 mmol) and DMF (10 mL). The reaction mixture was stirred at 20° C. for 10 minutes, then tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (80 mg, 1 Eq, 0.12 mmol) was added and the reaction mixture was stirred at 25° C. for an additional 1 hour. The mixture was directly purified by MPLC to provide 5-(6-(((benzyloxy)carbonyl)amino)hexanamido)pentanoic acid (300 mg, 823 μmol, 43.7%) as a yellow oil.

Step 2: 5-(6-(((benzyloxy)carbonyl)amino)hexanamido)pentanoic acid (51 mg, 1.2 Eq, 0.14 mmol) was treated with HATU (53 mg, 1.2 Eq, 0.14 mmol), DIEA (45 mg, 61 μL, 3.0 Eq, 0.35 mmol) and tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (80 mg, 1 Eq, 0.12 mmol) in a manner similar to Example 14, Step 2 to provide tert-butyl (1R,3 S,5r,7r)-2-(1-(4-(1-(5-(6-(((benzyloxy)carbonyl)amino)hexanamido)pentanoyl)piperidin-4-yl)-2-isopropylphenyl)-5-(2,6-dimethoxyphenyl)-1H-pyrazole-3-carboxamido)adamantane-2-carboxylate (70 mg, 68 μmol, 58%) as a yellow oil. [M+H]⁺=1030.9.

Steps 3 and 4: tert-butyl (1R,3S,5r,7r)-2-(1-(4-(1-(5-(6-(((benzyloxy)carbonyl)amino)hexanamido)pentanoyl)piperidin-4-yl)-2-isopropylphenyl)-5-(2,6-dimethoxyphenyl)-1H-pyrazole-3-carboxamido)adamantane-2-carboxylate was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2,2′-(10-(2-((6-((5-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)-5-oxopentyl)amino)-6-oxohexyl)amino)-2-oxoethyl)-1,4,7,10-tetraazacyclododecane-1,4,7-triyl)triacetic acid (57.7 mg, 43.1 μmol, 63%) as a white solid. MS: Calc'd for C₆₄H₉₂N₁₀O₄: 1224.68, found [M+H]⁺: 1225.6.

### Example 17: Compound 17

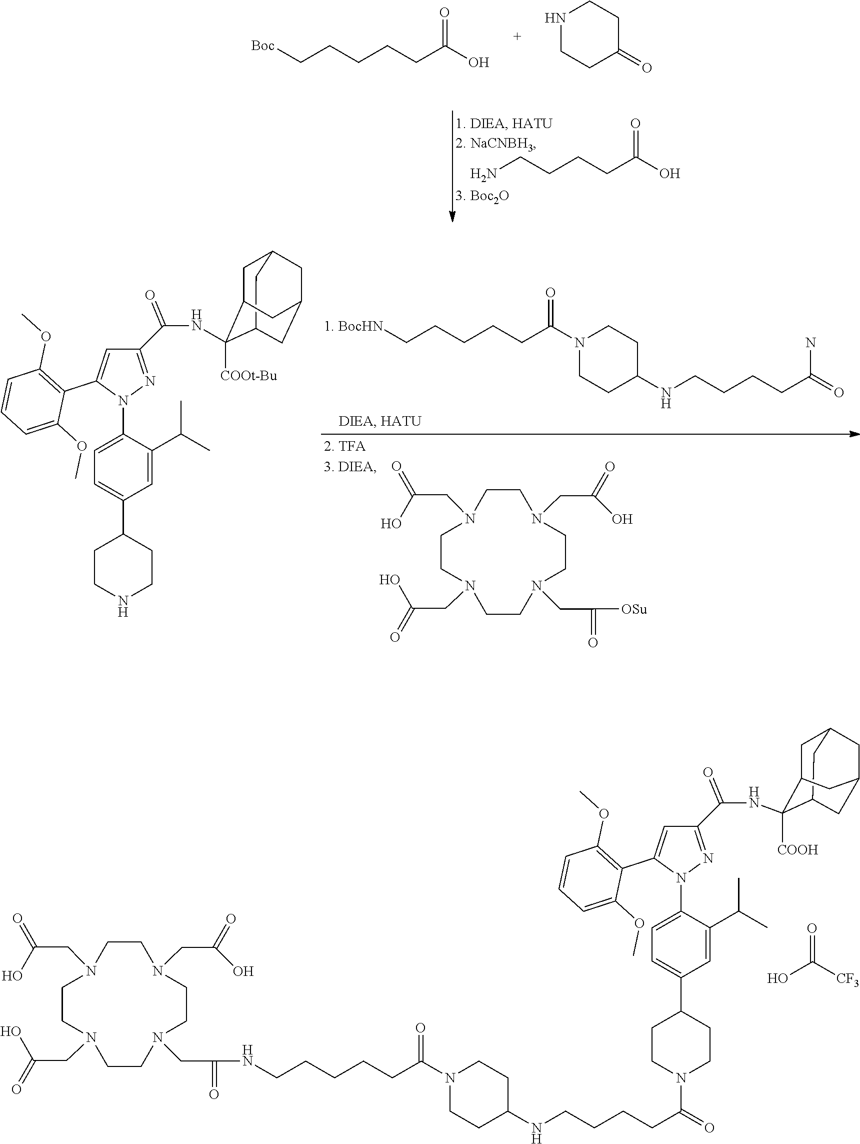

Step 1: 6-((tert-butoxycarbonyl)amino)hexanoic acid (1.0 g, 1 Eq, 4.3 mmol), was treated with HATU (2.0 g, 1.2 Eq, 5.3 mmol), DIEA (1.7 g, 2.3 mL, 3.0 Eq, 13 mmol) and piperidin-4-one (₅10 mg, 1.2 Eq, 5.14 mmol) in a manner similar to Example 14, Step 2 to provide tert-butyl (6-oxo-6-(4-oxopiperidin-1-yl)hexyl)carbamate (1.3 g, 4.2 mmol, 96%) as a yellow oil. [M+H]⁺=313.2.

Step 2: tert-butyl (6-oxo-6-(4-oxopiperidin-1-yl)hexyl)carbamate was treated 5-aminopentanoic acid (144 mg, 1.10 Eq, 1.23 mmol) and sodium cyanoborohydride (141 mg, 131 μL, 2.00 Eq, 2.24 mmol) in a manner similar to Example 14, Step 1 to provide 5-((1-(6-((tert-butoxycarbonyl)amino)hexanoyl)piperidin-4-yl)amino)pentanoic acid (450 mg, 1.09 mmol, 97.1%) as a light yellow solid. [M+H]⁺=414.3.

Step 3: Into an 8-mL vial, was placed a mixture of 5-((1-(6-((tert-butoxycarbonyl)amino)hexanoyl)piperidin-4-yl)amino)pentanoic acid (400 mg, 1 Eq, 967 μmol) and EtOH (5 mL), to which was added di-tert-butyl dicarbonate (317 mg, 324 μL, 1.50 Eq, 1.45 mmol). The reaction mixture was stirred at 50° C. for 1 hour. The mixture was concentrated under reduced pressure and purified by MPLC to provide 5-((tert-butoxycarbonyl)(1-(6-((tert-butoxycarbonyl)amino)hexanoyl)piperidin-4-yl)amino)pentanoic acid (200 mg, 389 μmol, 40.3%) as a white solid. [M+H]⁺=514.3.

Step 4: tert-Butyl (1R,3 S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate was treated with 5-((tert-butoxycarbonyl)(1-(6-((tert-butoxycarbonyl)amino)hexanoyl)piperidin-4-yl)amino)pentanoic acid was treated with HATU (53 mg, 1.2 Eq, 0.14 mmol) and DIEA (45 mg, 61 μL, 3.0 Eq, 0.35 mmol) in a manner similar to Example 14, Step 2 to provide tert-butyl (1R,3S,5r,7r)-2-(1-(4-(1-(5-((tert-butoxycarbonyl)(1-(6-((tert-butoxycarbonyl)amino)hexanoyl)piperidin-4-yl)amino)pentanoyl)piperidin-4-yl)-2-isopropylphenyl)-5-(2,6-dimethoxyphenyl)-1H-pyrazole-3-carboxamido)adamantane-2-carboxylate (70 mg, 59 μmol, 51%) as a yellow oil. [M+H]⁺=1178.9.

Steps 5-6: The product from Step 4 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((6-(4-((5-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-5-oxopentyl)amino)piperidin-1-yl)-6-oxohexyl)amino)-2-oxoethyl)-1,4,7,10-tetraazacyclododecane-1,4,7-triyl)triacetic acid (47.5 mg, 33.4 mol, 56%) as a white solid. MS: Calc'd for C₆₉H₁₀₁N₁₁O₁₄: 1307.75, found [M+H]⁺: 1308.7.

### Example 18: Compound 18

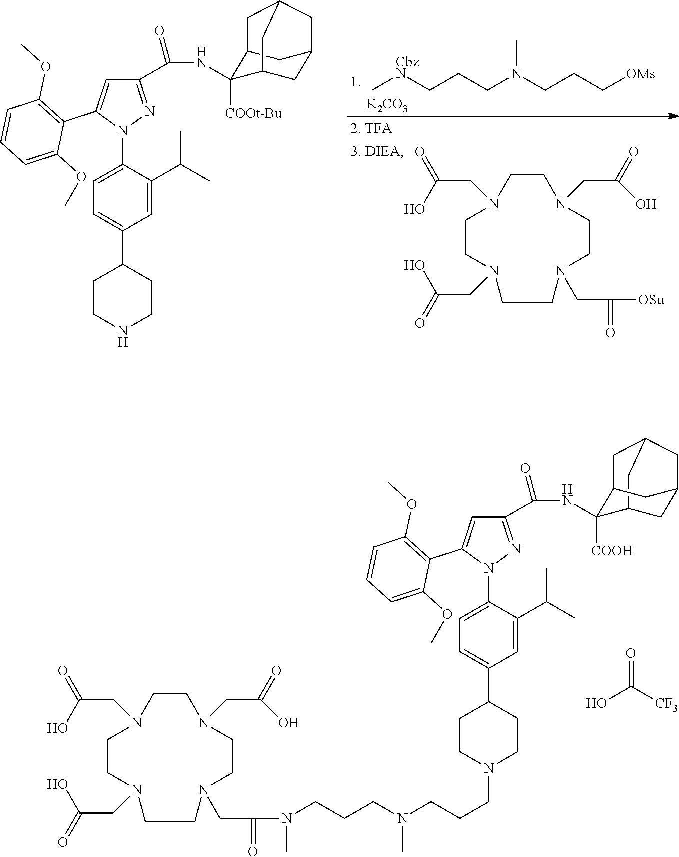

Step 1: tert-Butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate was treated with 3-((3-(((benzyloxy)carbonyl)(methyl)amino)propyl)(methyl)amino)propyl methanesulfonate (98 mg, 2.0 Eq, 0.26 mmol) and K₂CO₃ (55 mg, 3.0 Eq, 0.40 mmol) in a manner similar to Example 13, Step 3 to provide tert-butyl (1R,3S,5r,7r)-2-(1-(4-(1-(3-((3-(((benzyloxy)carbonyl)(methyl)amino)propyl)(methyl)amino)propyl)piperidin-4-yl)-2-isopropylphenyl)-5-(2,6-dimethoxyphenyl)-1H-pyrazole-3-carboxamido)adamantane-2-carboxylate (50 mg, 52 μmol, 40%) as a yellow oil. [M+H]⁺=959.6.

Steps 2-3: tert-butyl (1R,3S,5r,7r)-2-(1-(4-(1-(3-((3-(((benzyloxy)carbonyl)(methyl)amino)propyl)(methyl)amino)propyl)piperidin-4-yl)-2-isopropylphenyl)-5-(2,6-dimethoxyphenyl)-1H-pyrazole-3-carboxamido)adamantane-2-carboxylate was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)(methyl)amino)propyl)(methyl)amino)-2-oxoethyl)-1,4,7,10-tetraazacyclododecane-1,4,7-triyl)triacetic acid (12.6 mg, 10.9 μmol, 22%) as a white solid. MS: Calc'd for C₆₁H₉₀N₁₀O₁₂: 1154.67, found [M+H]⁺: 1155.7.

### Example 19: Compound 19

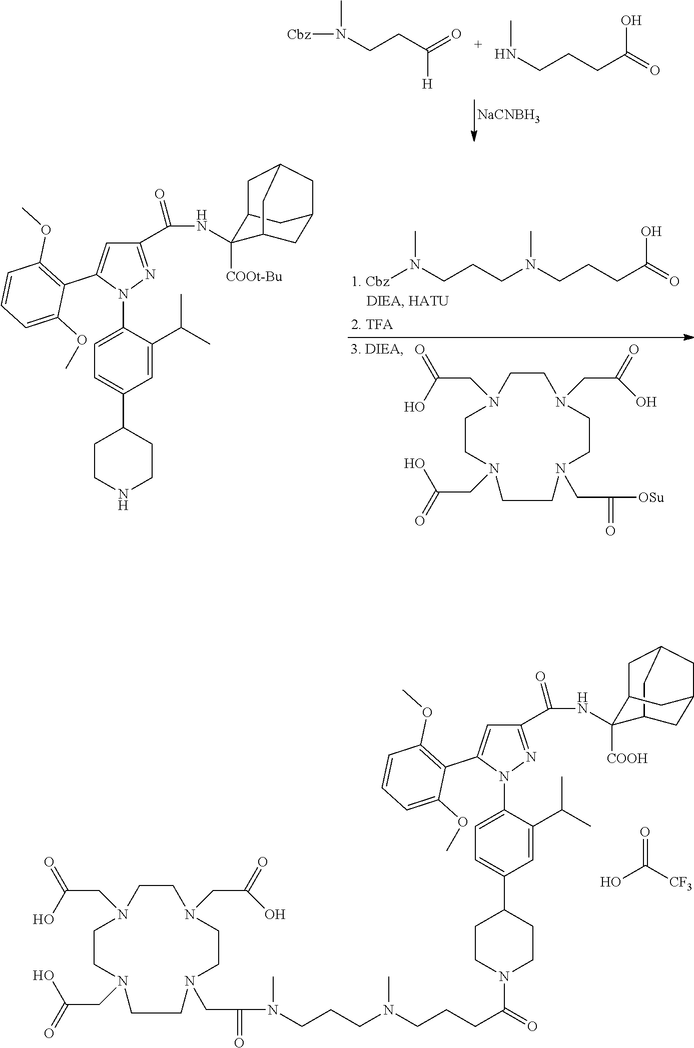

Step 1: Benzyl methyl(3-oxopropyl)carbamate (220 mg, 1 Eq, 994 μmol) and 4-(methylamino)butanoic acid (128 mg, 1.10 Eq, 1.09 mmol) were treated with NaBH-3CN (62.5 mg, 1 Eq, 994 μmol) in a manner similar to Example 14, Step 1 to provide 4-((3-(((benzyloxy)carbonyl)(methyl)amino)propyl)(methyl)amino)butanoic acid (150 mg, 465 μmol, 46.8(%) as a yellow oil. [M+H]⁺=323.2.

Step 2: 4-((3-(((benzyloxy)carbonyl)(methyl)amino)propyl)(methyl)amino)butanoc acid (60 mg, 1.5 Eq, 0.19 mmol) was treated with HATU (71 mg, 1.5 Eq, 0.19 mmol), DIEA (64 mg, 86 μL, 4.0 Eq, 0.50 mmol), and tert-butyl (1R,3S,5r,7r)-2-(5-(2,6-dimethoxyphenyl)-1-(2-isopropyl-4-(piperidin-4-yl)phenyl)-1H-pyrazole-3-carboxamnido)adamantane-2-carboxylate (85 mg, 1 Eq, 0.12 mmol) in a manner similar to Example 14, Step 2, to provide tert-butyl (1R,3 S,5r,7r)-2-(1-(4-(1-(4-((3-(((benzyloxy)carbonyl)(methyl)amino)propyl)(methyl)amino)butanoyl)piperidin-4-yl)-2-isopropylphenyl)-5-(2,6-dimethoxyphenyl)-1H-pyrazole-3-carboxamido)adamantane-2-carboxylate (53 mg, 54 μmol, 43%) as a yellow oil. [M+H]⁺987.8.

Steps 3-4: The product from step 2 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-4-oxobutyl)(methyl)amino)propyl)(methyl)amino)-2-oxoethyl)-1,4,7,10-tetraazacyclododecane-1,4,7-triyl)triacetic acid (30.1 mg, 23.2 μmol, 41%) as a white solid. MS: Calc'd for C₆₂H₉N₁₀O₁₃: 1182.67, found [M+H]⁺: 1183.9.

### Example 20: Compound 20

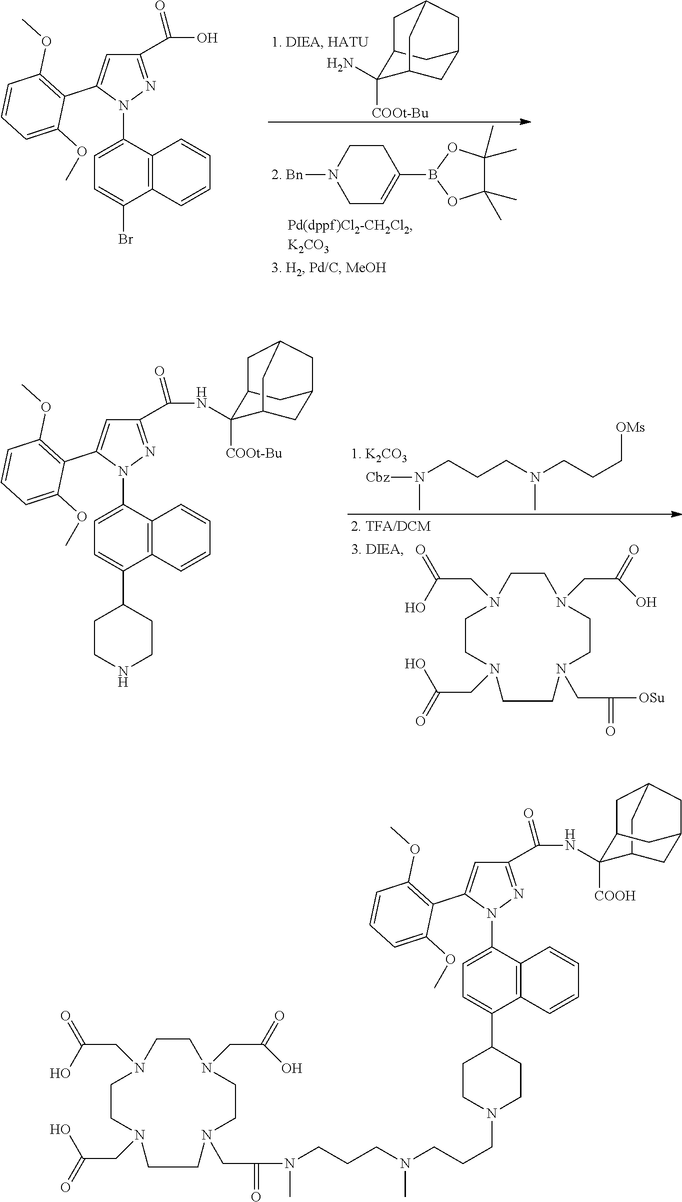

Steps 1-3: 1-(4-bromonaphthalen-1-yl)-5-(2,6-dimethoxyphenyl)-1H-pyrazole-3-carboxylic acid (200 mg, 1 Eq, 441 μmol) and tert-butyl (1R,3S,5r,7r)-2-aminoadamantane-2-carboxylate (133 mg, 1.20 Eq, 529 μmol) were treated with HATU and DIEA, then 1-benzyl-4-(4,4,5,5-tetramethyl-1,3,2-dioxaborolan-2-yl)-1,2,3,6-tetrahydropyridine and Pd(dppf)Cl₂—CH₂Cl₂, then hydrogen and Pd/C in a manner similar to Example 1, Steps 5-7 to provide tert-butyl (1R,3S,5r,7r)-2-(5-(2,6-dimethoxyphenyl)-1-(4-(piperidin-4-yl)naphthalen-1-yl)-1H-pyrazole-3-carboxamido)adamantane-2-carboxylate (160 mg, 232 μmol, 82.0%) as a white solid. [M+H]⁺691.9.

Step 4: The product from Step 3 was treated with 3-((3-(((benzyloxy)carbonyl)(methyl)amino)propyl)(methyl)amino)propyl methanesulfonate and K₂CO₃ in a manner similar to Example 13, Step 3 to provide tert-butyl (1R,3S,5r,7r)-2-(1-(4-(1-(3-((3-(((benzyloxy)carbonyl)(methyl)amino)propyl)(methyl)amino)propyl)piperidin-4-yl)naphthalen-1-yl)-5-(2,6-dimethoxyphenyl)-1H-pyrazole-3-carboxamido)adamantane-2-carboxylate (40 mg, 41 μmol, 36%) as a yellow oil. [M+H]⁺:967.6.

Steps 5-6: The product from Step 4 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((3-(4-(4-(5-(2,6-dimethoxyphenyl)-3-(((1R,3S,5r,7r)-2-(hydroxy(oxo)-15-methyl)adamantan-2-yl)carbamoyl)-1H-pyrazol-1-yl)naphthalen-1-yl)piperidin-1-yl)propyl)(methyl)amino)propyl)(methyl)amino)-2-oxoethyl)-1,4,7,10-tetraazacyclododecane-1,4,7-triyl)triacetic acid-2,2,2-trifluoroacetic acid (1/1) (26.1 mg, 20.4 μmol, 53%) as a white solid. MS: Calc'd for C₆₂H₈₆N₁₀O₁₂: 1162.64, found [M+H]⁺: 1163.7.

### Example 21: Compound 21

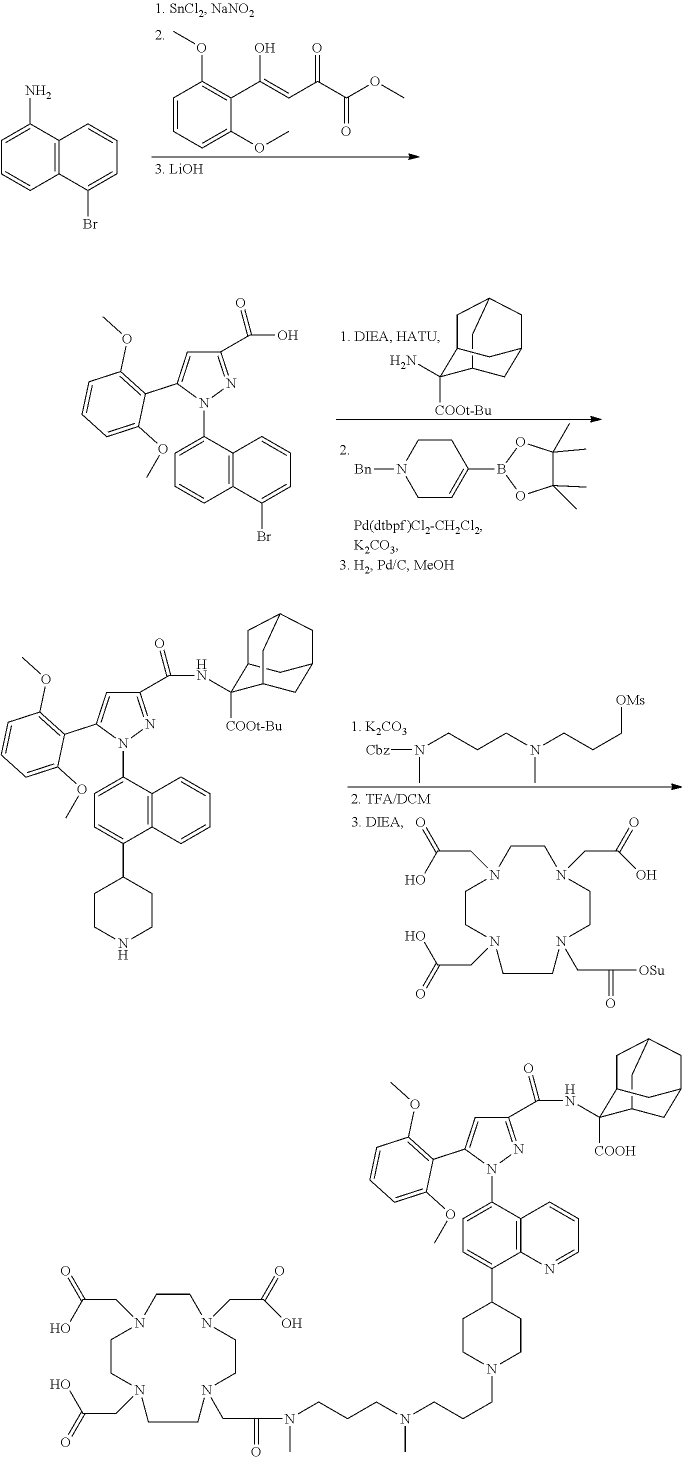

Step 1: Into a 250-mL vial, was placed a mixture of 5-bromonaphthalen-1-amine (1 g, 1 Eq, 5 mmol), sodium nitrite (0.6 g, 2 Eq, 9 mmol), HCl (10 mL) and H₂O (5 mL). The reaction mixture was stirred at 0° C. for 1 hour, then tin(II) chloride dihydrate (4.0 g, 4 Eq, 18 mmol) and HCl (10 mL) were added and the reaction mixture was stirred at 25° C. for an additional 2 hours. The mixture was quenched with Et₂O (25 mL), the reaction mixture was filtered through a pad of celite to afford (5-bromonaphthalen-1-yl)hydrazine (1.1 g, 4.6 mmol) as a light yellow solid, which was used directly in the next step without any purification.

Step 2: (5-bromonaphthalen-1-yl)hydrazine was treated with methyl (Z)-4-(2,6-dimethoxyphenyl)-4-hydroxy-2-oxobut-3-enoate (1.5 g, 1.2 Eq, 5.6 mmol) in a manner similar to Example 1, Step 3 to provide methyl 1-(5-bromonaphthalen-1-yl)-5-(2,6-dimethoxyphenyl)-1H-pyrazole-3-carboxylate (2 g, 4 mmol, 90%) as a light yellow solid.

Steps 3-6: tert-butyl (1R,3S,5r,7r)-2-(5-(2,6-dimethoxyphenyl)-1-(5-(piperidin-4-yl)naphthalen-1-yl)-1H-pyrazole-3-carboxamido)adamantane-2-carboxylate was synthesized from methyl 1-(5-bromonaphthalen-1-yl)-5-(2,6-dimethoxyphenyl)-1H-pyrazole-3-carboxylate in a manner similar to Example 1, Steps 4-7 to provide the product (450 mg, 651 μmol, 101%) as a white oil. [M+H]⁺=691.7.

Steps 7-9: The product from Step 6 was treated with 3-((3-(((benzyloxy)carbonyl)(methyl)amino)propyl)(methyl)amino)propyl methanesulfonate, then TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 3-5 to provide the 2,2,2-trifluoroacetic acid salt of 2,2′,2″-(10-(2-((3-((3-(4-(5-(5-(2,6-dimethoxyphenyl)-3-(((1R,3S,5r,7r)-2-(hydroxy(oxo)-15-methyl)adamantan-2-yl)carbamoyl)-1H-pyrazol-1-yl)naphthalen-1-yl)piperidin-1-yl)propyl)(methyl)amino)propyl)(methyl)amino)-2-oxoethyl)-1,4,7,10-tetraazacyclododecane-1,4,7-triyl)triacetic acid (23.3 mg, 18.2 μmol, 25%) as an off-white solid. MS: Calc'd for C₆₂H₈₆N₁₀O₁₂: 1162.64, found [M+H]⁺: 1163.7.

### Example 22: Compound 22

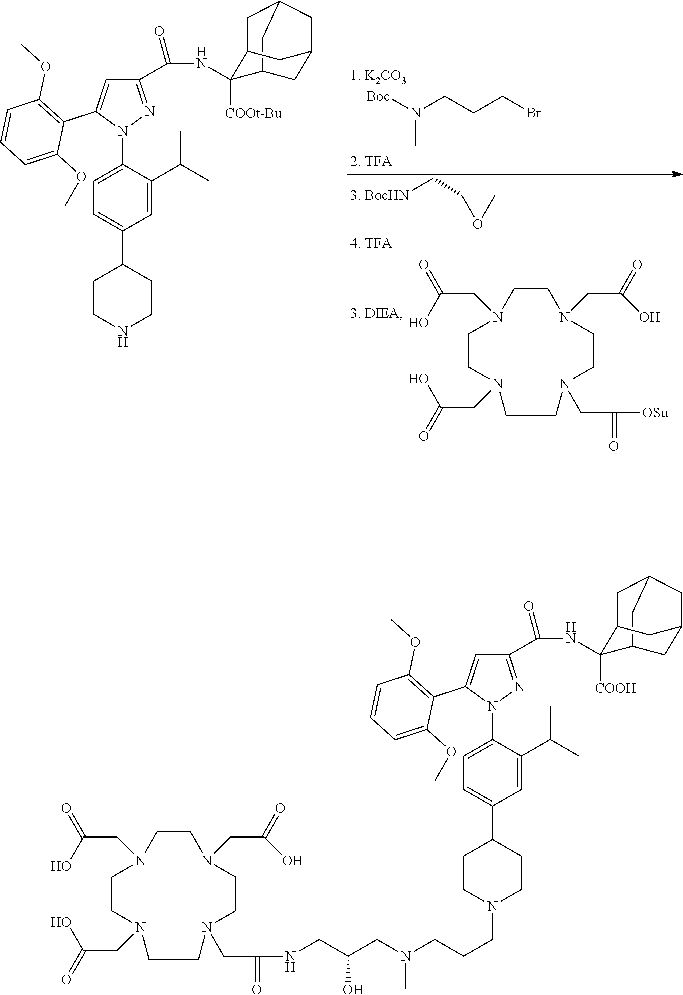

Step 1: Into an 8-mL vial, was placed a mixture of tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (100 mg, 1 Eq, 146 μmol), K₂CO₃ (60 mg, 3.0 Eq, 0.43 mmol), tert-butyl (3-bromopropyl)(methyl)carbamate (111 mg, 3.01 Eq, 440 μmol) and DMF (1 mL). The reaction mixture was stirred at 80° C. for 3 hours then the mixture was directly purified by MPLC to provide tert-butyl (1R,3S,5r,7r)-2-(1-(4-(1-(3-((tert-butoxycarbonyl)(methyl)amino)propyl)piperidin-4-yl)-2-isopropylphenyl)-5-(2,6-dimethoxyphenyl)-1H-pyrazole-3-carboxamido)adamantane-2-carboxylate (100 mg, 117 μmol, 80.0%) as a yellow oil. [M+H]⁺854.5.

Step 2: The product from Step 1 was treated with TFA in a manner similar to Example 13, Step 4 to provide (1R,3S,5r,7r)-2-(5-(2,6-dimethoxyphenyl)-1-(2-isopropyl-4-(1-(3-(methylamino)propyl)piperidin-4-yl)phenyl)-1H-pyrazole-3-carboxamido)adamantane-2-carboxylic acid (80 mg, 0.11 mmol, 98%) as a yellow oil which was directly used in the next step without any purification. [M+H]⁺=698.4.

Step 3: Into an 8-mL vial, was placed a mixture of (1R,3S,5r,7r)-2-(5-(2,6-dimethoxyphenyl)-1-(2-isopropyl-4-(1-(3-(methylamino)propyl)piperidin-4-yl)phenyl)-1H-pyrazole-3-carboxamido)adamantane-2-carboxylic acid (40 mg, 1 Eq, 57 μmol), tert-butyl (R)-(oxiran-2-ylmethyl)carbamate (50 mg, 5.0 Eq, 0.29 mmol) and EtOH (1 mL) and the reaction mixture was stirred at 40° C. for 16 hours. The crude mixture was directly purified by Prep-HPLC to provide (1R,3S,5r,7r)-2-(1-(4-(1-(3-(((R)-3-((tert-butoxycarbonyl)amino)-2-hydroxypropyl)(methyl)amino)propyl)piperidin-4-yl)-2-isopropylphenyl)-5-(2,6-dimethoxyphenyl)-1H-pyrazole-3-carboxamido)adamantane-2-carboxylic acid (20 mg, 23 μmol, 40%) as a white solid.

Steps 4-5: The product from Step 3 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″(10-(2-(((R)-3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)(methyl)amino)-2-hydroxypropyl)amino)-2-oxoethyl)-1,4,7,10-tetraazacyclododecane-1,4,7-triyl)triacetic acid (1.1 mg, 0.87 μmol, 3.3%) as a white solid. MS: Calc'd for C₆₀H₈₈N₁₀O₁₃: 1156.65, found [M+H]⁺: 1157.6.

### Example 23: Compound 23

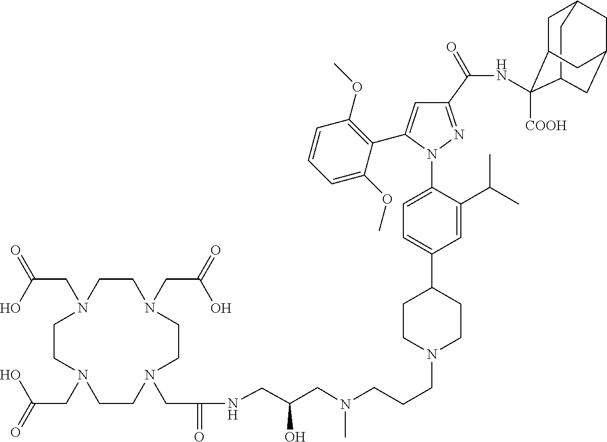 [SMILES: COc1cccc(OC)c1-c1cc(C(=O)NC2(C(=O)O)C3CC4CC(C3)CC2C4)nn1-c1ccc(C2CCN(CCCN(C)C[C@@H](O)CNC(=O)CN3CCN(CC(=O)O)CCN(CC(=O)O)CCN(CC(=O)O)CC3)CC2)cc1C(C)C]

Compound 23 was synthesized in a manner similar to Compound 22, using tert-butyl (S)-(oxiran-2-ylmethyl)carbamate in Step 3 to provide the trifluoroacetic acid salt of 2,2′,2″-(10-(2-(((S)-3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)(methyl)amino)-2-hydroxypropyl)amino)-2-oxoethyl)-1,4,7,10-tetraazacyclododecane-1,4,7-triyl)triacetic acid (3.4 mg, 2.7 μmol, 13%) as a white solid. MS: Calc'd for C₆₀H₈₈N₁₀O₁₃: 1156.65, found [M+H]⁺: 1157.7.

### Example 24: Compound 24

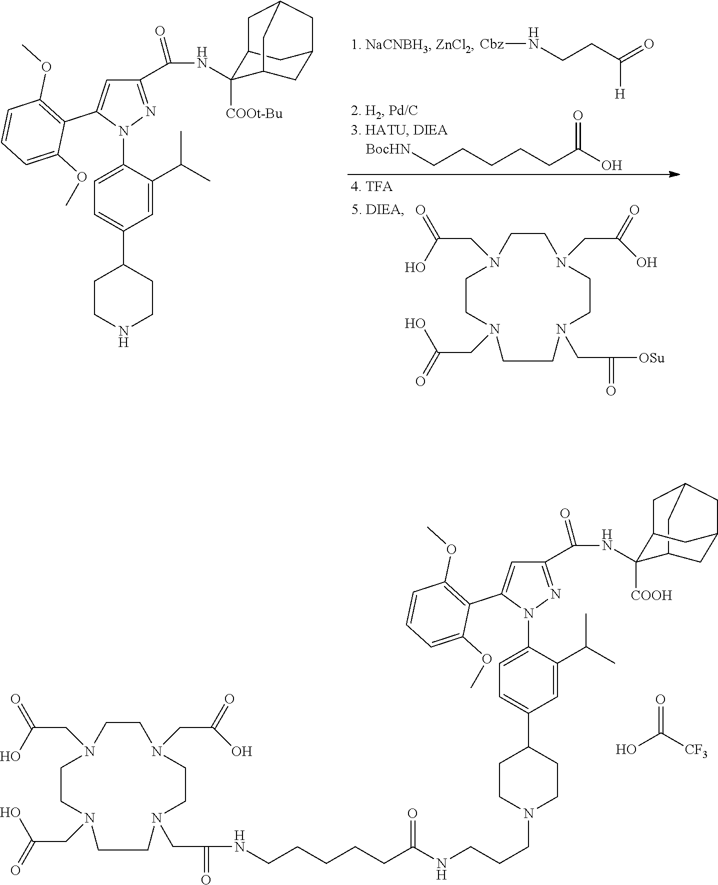

Step 1: Into a 40-mL vial, was placed a mixture of tert-butyl (1R,3 S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (500 mg, 1 Eq, 732 μmol), benzyl (3-oxopropyl)carbamate (182 mg, 1.20 Eq, 878 μmol), zinc(II) chloride (200 mg, 2.00 Eq, 1.47 mmol) and MeOH (6 mL). The reaction mixture was stirred at 20° C. for 30 minutes, then NaBH₃CN (92 mg, 2.0 Eq, 1.5 mmol) was added and the reaction mixture was stirred at 25° C. for an additional 1 hour. The mixture was directly purified by MPLC to provide tert-butyl (1R,3S,5r,7r)-2-(1-(4-(1-(3-(((benzyloxy)carbonyl)amino)propyl)piperidin-4-yl)-2-isopropylphenyl)-5-(2,6-dimethoxyphenyl)-1H-pyrazole-3-carboxamido)adamantane-2-carboxylate (500 mg, 572 mol, 78.1%) as a yellow oil.

Step 2: Into a 100-mL round-bottom flask under an atmosphere of nitrogen, was placed tert-butyl (1R,3S,5r,7r)-2-(1-(4-(1-(3-(((benzyloxy)carbonyl)amino)propyl)piperidin-4-yl)-2-isopropylphenyl)-5-(2,6-dimethoxyphenyl)-1H-pyrazole-3-carboxamido)adamantane-2-carboxylate (500 mg, 1 Eq, 572 μmol) and MeOH (6 mL), to which was carefully added Pd/C (60 mg, 0.99 Eq, 0.56 mmol). The flask was evacuated and flushed with hydrogen three times. The mixture was stirred for 25° C. at 1 hour. The reaction mixture was filtered through a pad of celite, to provide tert-butyl (1R,3S,5r,7r)-2-(1-(4-(1-(3-aminopropyl)piperidin-4-yl)-2-isopropylphenyl)-5-(2,6-dimethoxyphenyl)-1H-pyrazole-3-carboxamido)adamantane-2-carboxylate (400 mg, 541 μmol, 94.5%) as a white solid. [M+H]⁺=740.7.

Steps 3-5: The product from Step 2 was treated with 6-((tert-butoxycarbonyl)amino)hexanoic acid, HATU, and DIEA, then TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 14, Step 1 and Example 13, Steps 3-4 to provide the 2,2,2-trifluoroacetic acid salt of 2,2′,2″-(10-(2-((6-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid (43.8 mug, 33.8 μmol, 77%) as a white solid. MS: Calc'd for C₆₂H₉₀N₁₀O₁₃: 1182.67, found [M+H]⁺: 1183.6.

### Synthesis of Compound 24-Lu

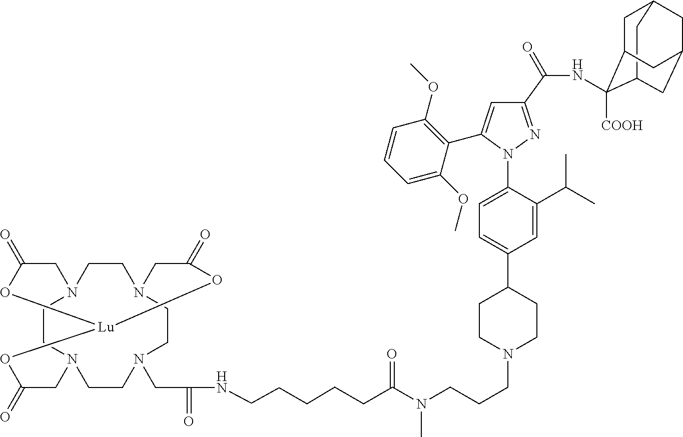

A mixture of 2,2′,2″-(10-(2-((6-((3-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid (25 mg, 1 Eq, 21 μmol), Lutetium (III) chloride (24 mg, 6.0 μL, 4.0 Eq, 85 μmol) and NaOAc/AcOH Buffer (2 mL) was stirred at 70° C. for 30 mi. The crude mixture was directly purified by Prep-HPLC to provide the product (18.3 mg, 13.5 mol, 64%) as a light blue solid. MS: Calc'd for C₆₂H₈₇LuN₁₀O₁₃: 1354.59, found [M+H]⁺: 1355.6.

### Example 25: Compound 25

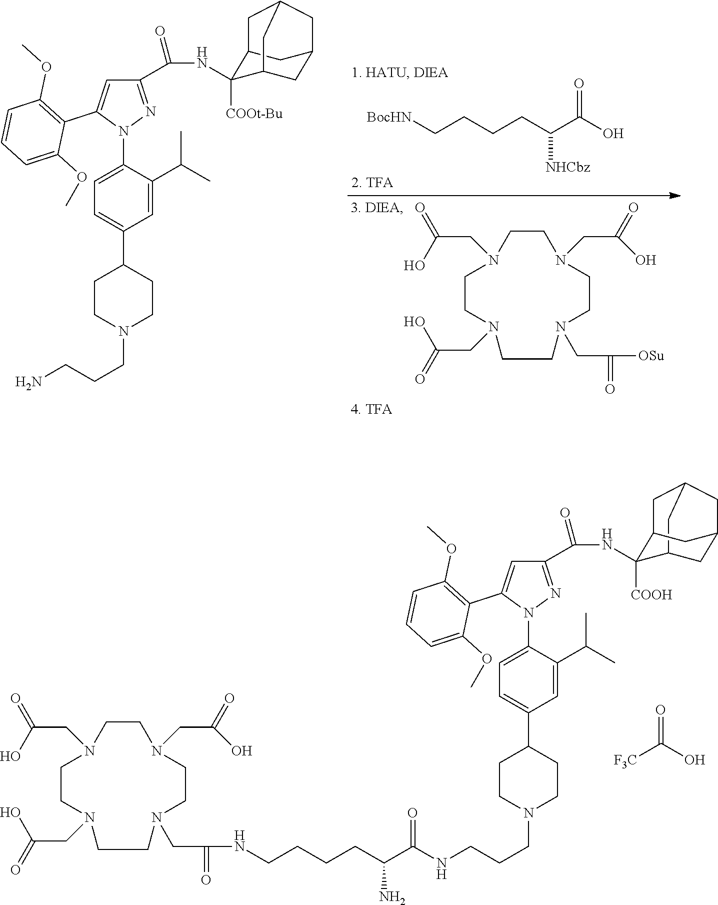

The product was synthesized from tert-butyl (1R,3S,5r,7r)-2-(1-(4-(1-(3-aminopropyl)piperidin-4-yl)-2-isopropylphenyl)-5-(2,6-dimethoxyphenyl)-1H-pyrazole-3-carboxamido)adamantane-2-carboxylate in a manner similar to Example 14 Step 2, Example 13 Steps 4-5, then Example 13 Step 4 to provide the trifluoroacetic acid salt of 2,2′,2″-(10-(2-(((R)-5-amino-6-((3-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid (19.5 mg, 14.9 μmol, 79%) as a white solid. MS: Calc'd for C₆₂H₉₁N₁₁O₁₃: 1197.68, found [M+H]⁺: 1198.6.

### Example 26: Compound 26

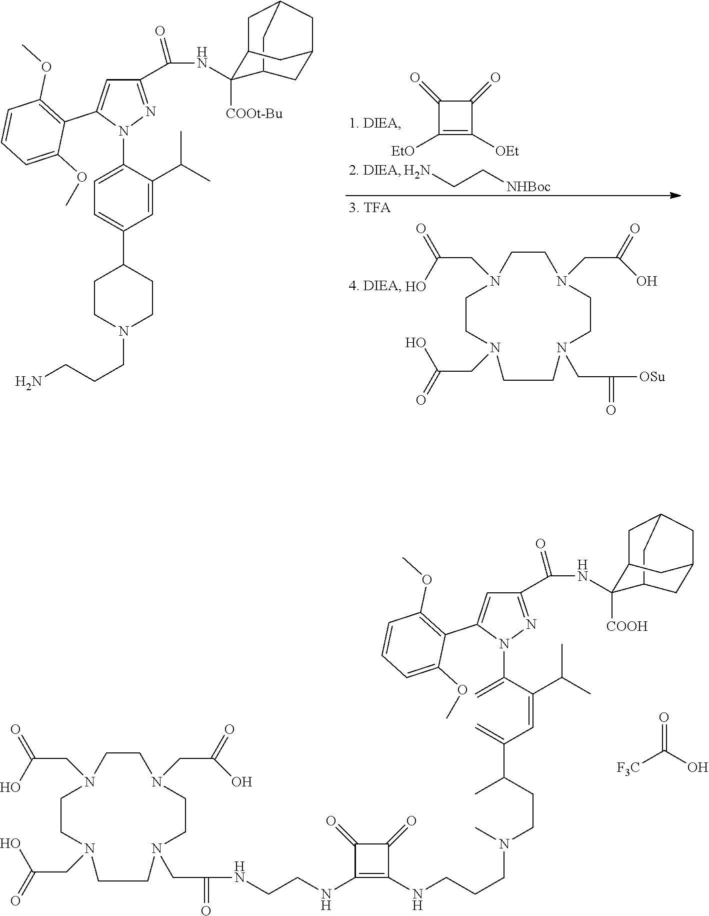

Step 1: Into a 40-mL vial, was placed a mixture of 3,4-diethoxycyclobut-3-ene-1,2-dione (12 mg, 1.2 Eq, 71 μmol), diisopropylethylamine (24 mg, 32 μL, 3.1 Eq, 0.19 mmol) and DMF (5 mL). The reaction mixture was stirred at 20° C. for 1 minute, then tert-butyl (1R,3S,5r,7r)-2-(1-(4-(1-(3-aminopropyl)piperidin-4-yl)-2-isopropylphenyl)-5-(2,6-dimethoxyphenyl)-1H₁-pyrazole-3-carboxamido)adamantane-2-carboxylate (45 mg, 1 Eq, 61 μmol) was added and the reaction mixture was stirred at 25° C. for an additional 1 hour. The mixture was directly purified by MPLC to provide tert-butyl (1R,35S,5r,7r)-2-(5-(2,6-dimethoxyphenyl)-1-(4-(1-(3-((2-ethoxy-3,4-dioxocyclobut-1-en-1-yl)amino)propyl)piperidin-4-yl)-2-isopropylphenyl)-1H-pyrazole-3-carboxamido)adamantane-2-carboxylate (40 mg, 46 μmol, 76%) as a yellow oil.

Step 2: Into an 8-mL vial, was placed a mixture of tert-butyl (2-aminoethyl)carbamate (15 mg, 2.0 Eq, 94 μmol), diisopropylethylamine (12 mg, 16 μL, 2.0 Eq, 93 μmol) and DMF (5 mL). The reaction mixture was stirred at 20° C. for 2 minutes, then tert-butyl (1R,3S,5r,7r)-2-(1-(4-(1-(3-aminopropyl)piperidin-4-yl)-2-isopropylphenyl)-5-(2,6-dimethoxyphenyl)-11H-pyrazole-3-carboxamido)adamantane-2-carboxylate (45 mg, 1 Eq, 61 kmol) was added and the reaction mixture was stirred at 25° C. for an additional 1 hour. The mixture was directly purified by MPLC to provide tert-butyl (1R,3S,5r,7r)-2-(1-(4-(1-(3-((2-((2-((tert-butoxycarbonyl)amino)ethyl)amino)-3,4-dioxocyclobut-1-en-1-yl)amino)propyl)piperidin-4-yl)-2-isopropylphenyl)-5-(2,6-dimethoxyphenyl)-1H-pyrazole-3-carboxamido)adamantane-2-carboxylate (28 mg, 29 μmol, 62%) as a yellow oil.

Steps 3-4: The product from Step 2 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((2-((2-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-3,4-dioxocyclobut-1-en-1-yl)amino)ethyl)amino)-2-oxoethyl)-1,4,7,10-tetraazacyclododecane-1,4,7-triyl)triacetic acid (26.4 mg, 20.0 μmol, 66%) as an off-white solid. MS: Calc'd for C₆₂H₈₅N₁₁O₁₄: 1207.63, found [M+H]v: 1208.7.

### Example 27: Compound 27

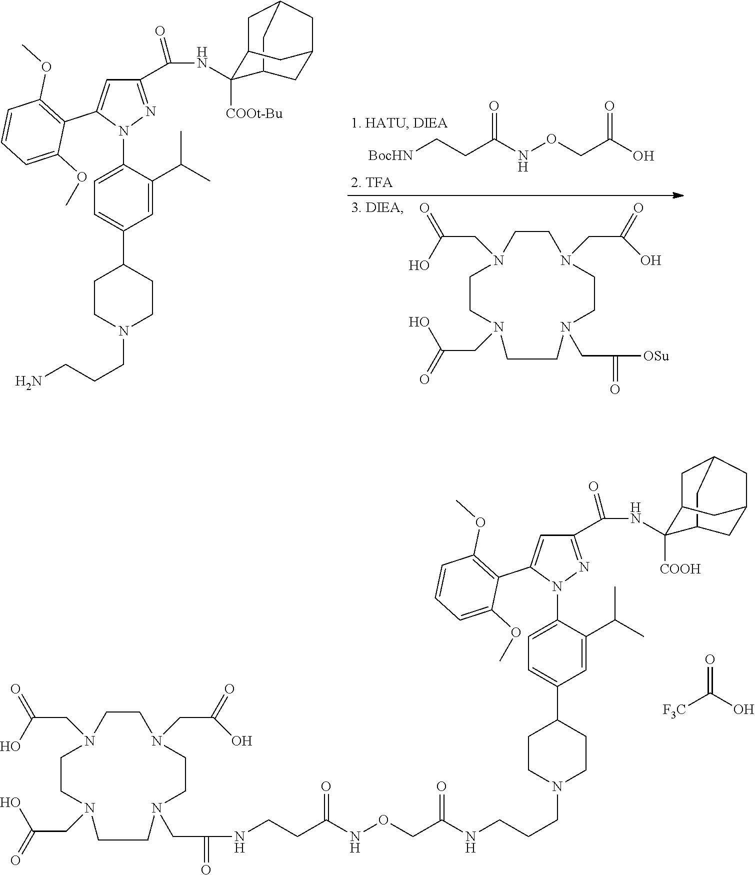

The product was synthesized from tert-butyl (1R,3S,5r,7r)-2-(1-(4-(1-(3-aminopropyl)piperidin-4-yl)-2-isopropylphenyl)-5-(2,6-dimethoxyphenyl)-1H-pyrazole-3-carboxamido)adamantane-2-carboxylate in a manner similar to Example 14 Step 2, then Example 13 Steps 4-5 to provide the trifluoroacetic acid salt of 2,2′,2″-(10-(14-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-2,6,10-trioxo-8-oxa-3,7,11-triazatetradecyl)-1,4,7,10-tetraazacyclododecane-1,4,7-triyl)triacetic acid as a white solid. MS: Calc'd for C₆₁H₈₇N₁₁O₁₅: 1213.64, found [M+H]⁺: 1241.7.

### Example 28: Compound 28

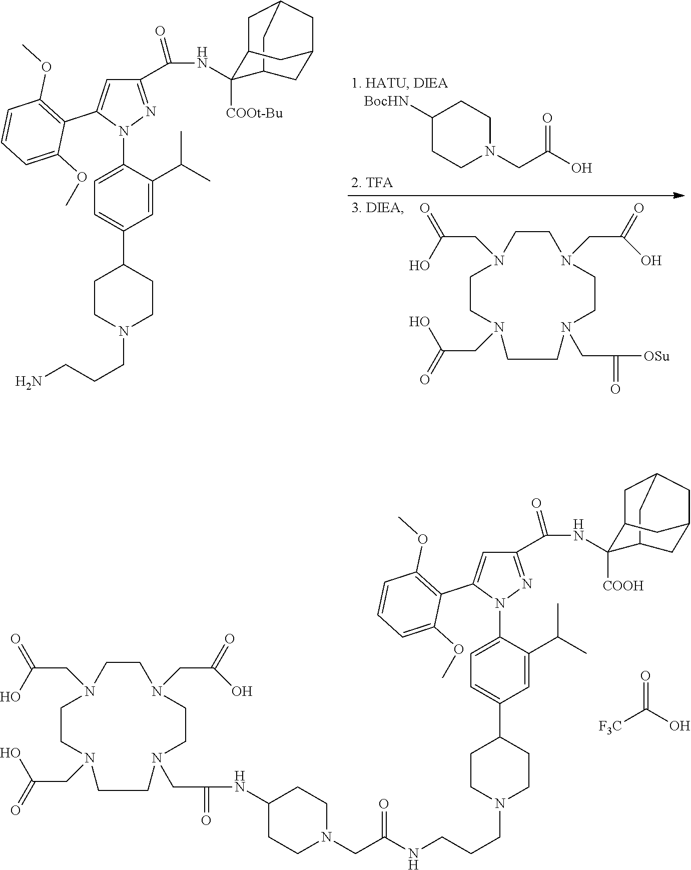

The product was synthesized from tert-butyl (1R,3 S,5r,7r)-2-(1-(4-(1-(3-aminopropyl)piperidin-4-yl)-2-isopropylphenyl)-5-(2,6-dimethoxyphenyl)-1H-pyrazole-3-carboxamido)adamantane-2-carboxylate in a manner similar to Example 14 Step 2, then Example 13 Steps 4-5 to provide the trifluoroacetic acid salt of 2,2′,2″-(10-(2-((1-(2-((3-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-2-oxoethyl)piperidin-4-yl)amino)-2-oxoethyl)-1,4,7,10-tetraazacyclododecane-1,4,7-triyl)triacetic acid (29.4 mg, 22.2 mol, 41%) as a white solid. MS: Calc'd for C₆₃H₉₁N₁₁O₁₃: 1209.68, found [M+H]⁺: 1210.7.

### Example 29: Compound 29

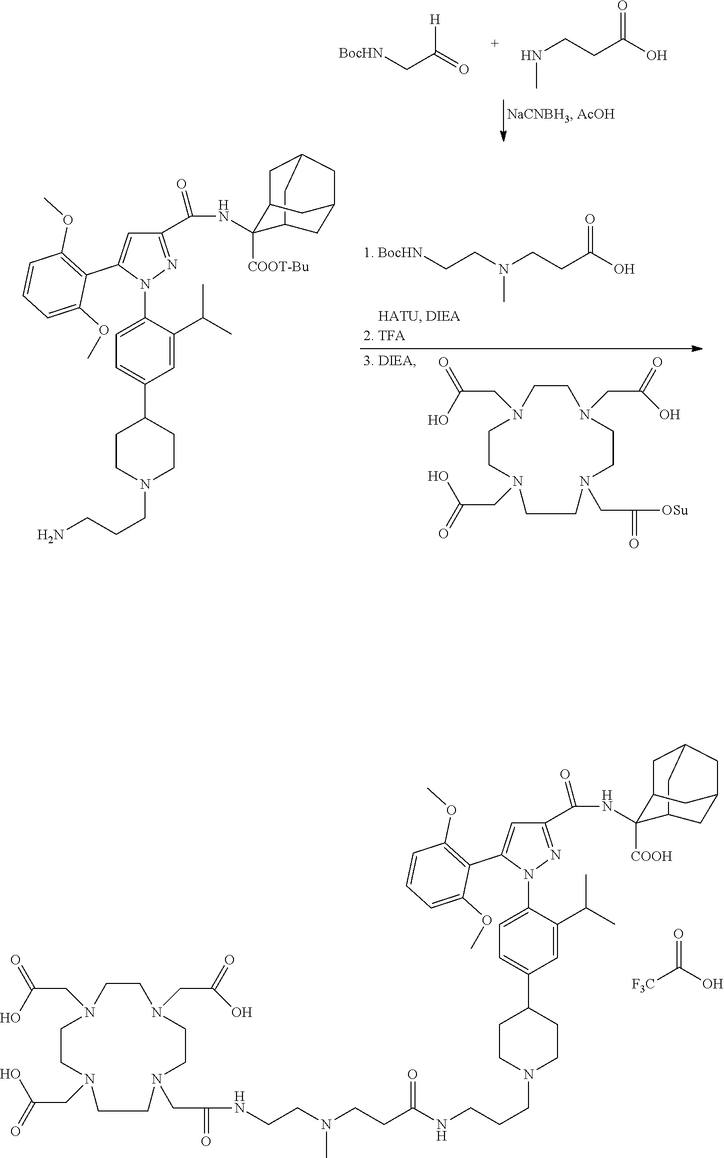

Step 1: tert-Butyl (2-oxoethyl)carbamate and 3-(methylamino)propanoic acid were treated with sodium cyanoborohydride in a manner similar to Example 14, Step 1 to provide 3-((2-((tert-butoxycarbonyl)amino)ethyl)(methyl)amino)propanoic acid (201 mg, 816 mol, 65.0%) as a yellow oil.

Steps 2-4: The product was synthesized from tert-butyl (1R,3S,5r,7r)-2-(1-(4-(1-(3-aminopropyl)piperidin-4-yl)-2-isopropylphenyl)-5-(2,6-dimethoxyphenyl)-1H-pyrazole-3-carboxamido)adamantane-2-carboxylate and 3-((2-((tert-butoxycarbonyl)amino)ethyl)(methyl)amino)propanoic acid in a manner similar to Example 14 Step 2, then Example 13 Steps 4-5 to provide the trifluoroacetic acid salt of 2,2′,2″-(10-(2-((2-((3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-3-oxopropyl)(methyl)amino)ethyl)amino)-2-oxoethyl)-1,4,7,10-tetraazacyclododecane-1,4,7-triyl)triacetic acid (15.1 mg, 11.5 μmol, 25%) as a white solid. MS: Calc'd for C₆₂H₉₁N₁₁O₁₃: 1197.68, found [M+H]⁺: 1198.8.

### Example 30: Compound 30

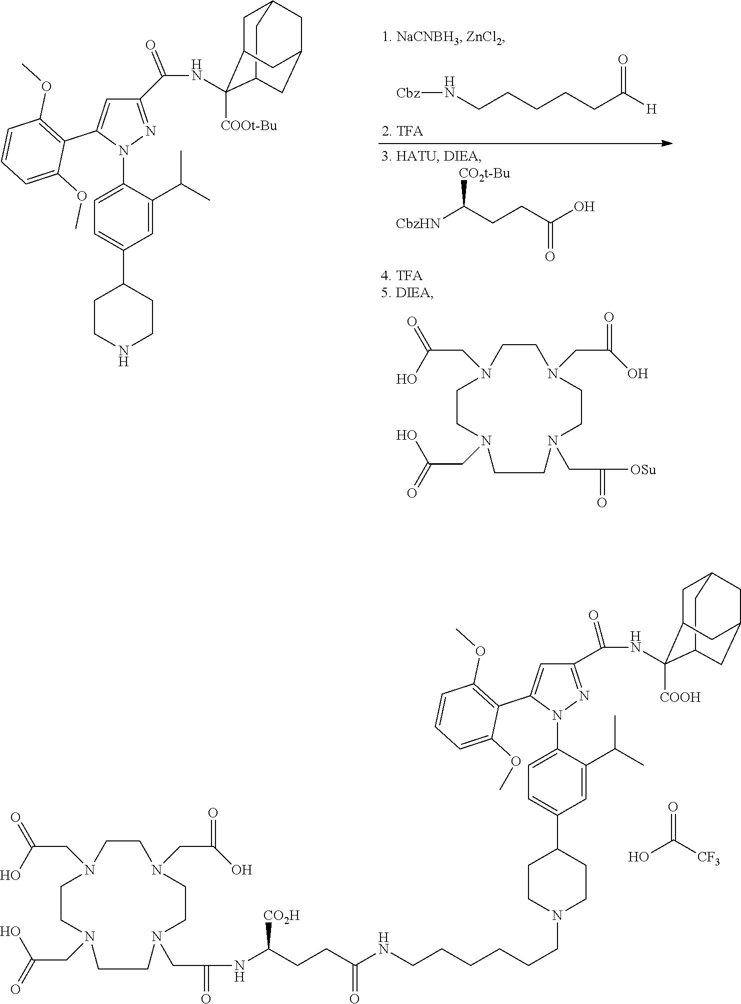

Step 1: tert-Butyl (1R,3 S,5r,7r)-2-(5-(2,6-dimethoxy phenyl)-1-(2-isopropyl-4-(piperidin-4-yl)phenyl)-1H-pyrazole-3-carboxamido)adamantane-2-carboxylate (200 mg, 1 Eq, 293 μmol) and benzyl (6-oxohexyl)carbamate (110 mg, 1.51 Eq, 441 μmol) were treated with zinc(II) chloride and NaBH₃CN in a manner similar to Example 24, Step 1 to provide tert-butyl (1R,3 S,5r,7r)-2-(1-(4-(1-(6-(((benzyloxy)carbonyl)amino)hexyl)piperidin-4-yl)-2-isopropylphenyl)-5-(2,6-dimethoxyphenyl)-1H-pyrazole-3-carboxamido)adamantane-2-carboxylate (140 mg, 153 μmol, 52.2%) as a yellow oil.

Step 2: The product from Step 1 was treated with TFA in a manner similar to Example 13, Steps 4 to provide (1R,3S,5r,7r)-2-(1-(4-(1-(6-aminohexyl)piperidin-4-yl)-2-isopropylphenyl)-5-(2,6-dimethoxyphenyl)-1H-pyrazole-3-carboxamido)adamantane-2-carboxylic acid (45 mg, 62 μmol, 76%) as a yellow oil.

Step 3: The product from Step 2 was treated with (R)-4-(((benzyloxy)carbonyl)amino)-5-(tert-butoxy)-5-oxopentanoic acid, H ATU and DIEA in a manner similar to Example 14, Step 2 to provide (1R,3 S,5r,7r)-2-(1-(4-(1-(6-((R)-4-(((benzyloxy)carbonyl)amino)-5-(tert-butoxy)-5-oxopentanamido)hexyl)piperidin-4-yl)-2-isopropylphenyl)-5-(2,6-dimethoxyphenyl)-1H-pyrazole-3-carboxamido)adamantane-2-carboxylic acid (30 mg, 29 μmol, 46%) as a yellow oil. [M+H]⁺=: 1045.7.

Steps 4-5: The product from Step 3 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-(((R)-1-carboxy-4-((6-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)hexyl)amino)-4-oxobutyl)amino)-2-oxoethyl)-1,4,7,10-tetraazacyclododecane-1,4,7-triyl)triacetic acid (4.4 mg, 2.8 μmol, 12%) as a white solid. MS: Calc'd for C₆₄H₉₂N₁₀O₁₅: 1240.67, found [M+H]⁺: 1241.9.

### Example 31: Compound 31

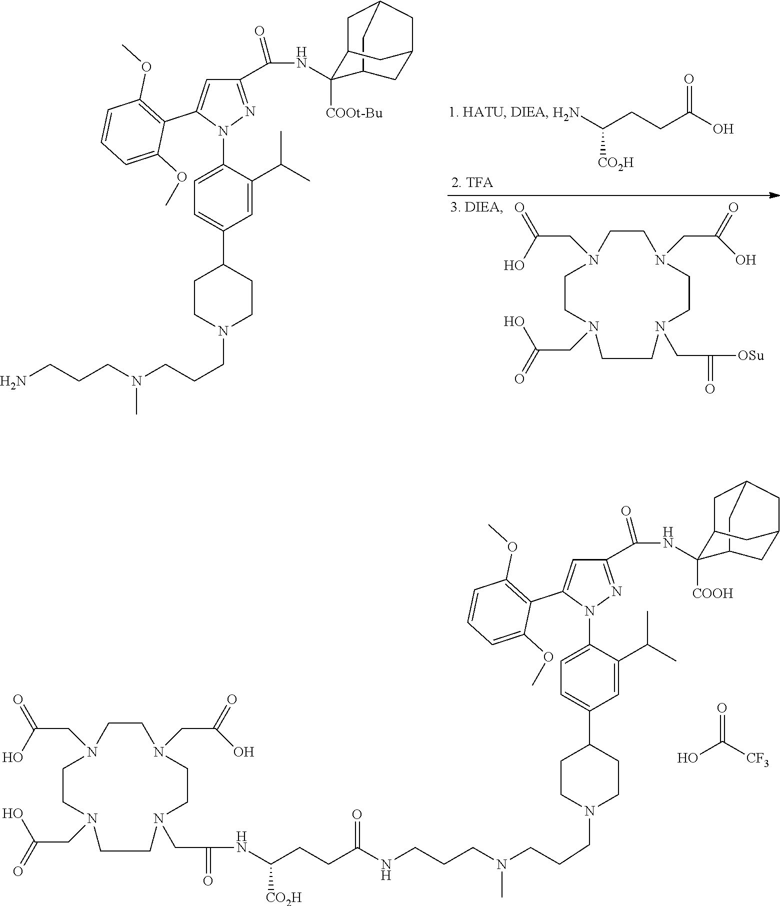

Step 1: (1R,3S,5r,7r)-2-(1-(4-(1-(3-((3-aminopropyl)(methyl)amino)propyl)piperidin-4-yl)-2-isopropylphenyl)-5-(2,6-dimethoxyphenyl)-1H-pyrazole-3-carboxamido)adamantane-2-carboxylic acid was treated with (R)-5-(tert-butoxy)-4-((tert-butoxycarbonyl)amino)-5-oxopentanoic acid, HATU and DIEA in a manner similar to Example 14, Step 2 to provide (1R,3 S,5r,7r)-2-(1-(4-(1-((R)-6-(tert-butoxycarbonyl)-2,2,14-trimethyl-4,9-dioxo-3-oxa-5,10,14-triazaheptadecan-17-yl)piperidin-4-yl)-2-isopropylphenyl)-5-(2,6-dimethoxyphenyl)-1H-pyrazole-3-carboxamido)adamantane-2-carboxylic acid (30 mg, 29 μmol, 54%) as a yellow oil. [M+H]⁺=1040.7.

Steps 2-3: The product from Step 1 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-(((R)-1-carboxy-4-((3-((3-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)(methyl)amino)propyl)amino)-4-oxobutyl)amino)-2-oxoethyl)-1,4,7,10-tetraazacyclododecane-1,4,7-triyl)triacetic acid (13.8 mg, 9.97 μmol, 35%) as a white solid. MS: Calc'd for C₆₅H₉₅N₁₀O₁₅: 1269.7, found [M+H]⁺: 1270.8.

### Example 32: Compound 32

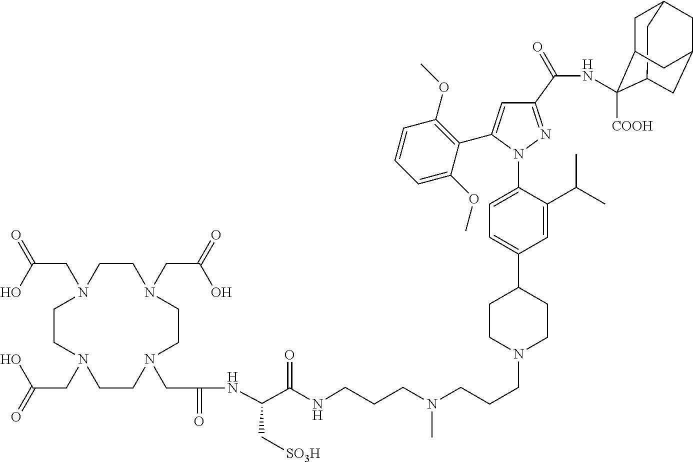 [SMILES: COc1cccc(OC)c1-c1cc(C(=O)NC2(C(=O)O)C3CC4CC(C3)CC2C4)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C]

The product was synthesized from (1R,3S,5r,7r)-2-(1-(4-(1-(3-((3-aminopropyl)(methyl)amino)propyl)piperidin-4-yl)-2-isopropylphenyl)-5-(2,6-dimethoxyphenyl)-1H-pyrazole-3-carboxamido)adamantane-2-carboxylic acid and (tert-butoxycarbonyl)(sulfo)-D-alanine in a manner similar to Example 14, Step 2 to provide 2,2′,2″-(10-(2-(((R)-1-((3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)(methyl)amino)propyl)amino)-1-oxo-3-sulfopropan-2-yl)amino)-2-oxoethyl)-1,4,7,10-tetraazacyclododecane-1,4,7-triyl)triacetic acid (18.6 mug, 13.2 μmol, 48%) as a white solid. MS: Calc'd for C₆₃H₉₃N₁O₁₆S: 1291.65, found [M+H]⁺: 1292.7.

### Synthesis of Compound 32-Lu

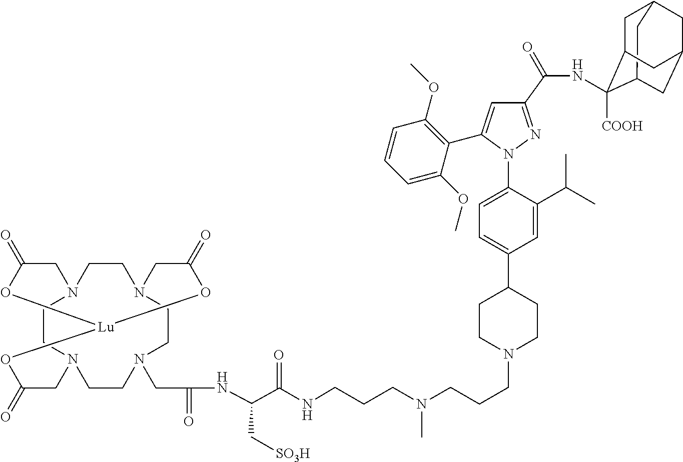

A mixture of 2,2′,2″-(10-(2-(((R)-1-((3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)(methyl)amino)propyl)amino)-3-(hydroxydioxo-17-sulfanyl)-1-oxopropan-2-yl)amino)-2-oxoethyl)-1,4,7,10-tetraazacyclododecane-1,4,7-triyl)triacetic acid (30 mg, 1 Eq, 23 μmol), lutetium (III) chloride (26 mg, 6.5 μL, 4.0 Eq, 92 μmol) and NaOAc/AcOH Buffer (2 mL) was stirred at 80° C. for 30 min. The crude mixture was directly purified by Prep-HPLC to provide the product (26.5 mg, 16.8 μmol, 72%) as a white solid. MS: Calc'd for C₆₃H₉₀LuN₁₁O₁₆S: 1463.57, found [M+H]⁺: 1464.5.

### Example 33: Compound 33

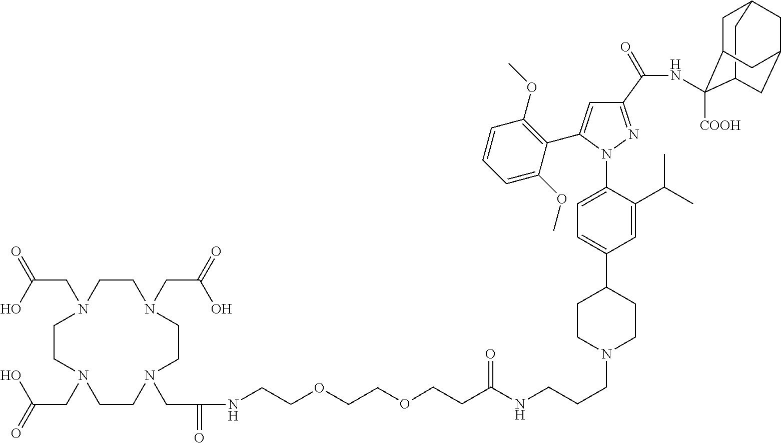 [SMILES: COc1cccc(OC)c1-c1cc(C(=O)NC2(C(=O)O)C3CC4CC(C3)CC2C4)nn1-c1ccc(C2CCN(CCCNC(=O)CCOCCOCCNC(=O)CN3CCN(CC(=O)O)CCN(CC(=O)O)CCN(CC(=O)O)CC3)CC2)cc1C(C)C]

tert-Butyl (1R,3S,5r,7r)-2-(1-(4-(1-(3-aminopropyl)piperidin-4-yl)-2-isopropylphenyl)-5-(2,6-dimethoxyphenyl)-1H-pyrazole-3-carboxamido)adamantane-2-carboxylate (from Example 24, Step 2) was treated with 2,2-dimethyl-4-oxo-3,8,11-trioxa-5-azatetradecan-14-oic acid, HATU and DIEA in a manner similar to Example 14, Step 2 to provide tert-butyl (1R,3 S,5r,7r)-2-(5-(2,6-dimethoxyphenyl)-1-(4-(1-(2,2-dimethyl-4,14-dioxo-3,8,11-trioxa-5,15-diazaoctadecan-18-yl)piperidin-4-yl)-2-isopropylphenyl)-1H-pyrazole-3-carboxamido)adamantane-2-carboxylate (28 mg, 28 μmol, 69%) as a yellow oil. [M+H]⁺=999.6.

Steps 2-4: The product from Step 1 was treated with TFA, then 2,2′,21″-(0-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(16-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-2,12-dioxo-6,9-dioxa-3,13-diazahexadecyl)-1,4,7,10-tetraazacyclododecane-1,4,7-triyl)triacetic acid (20.9 mg, 15.6 μmol, 52%) as a white solid. MS: Calc'd for C₆₃H₉₂N₁₀O₁₅: 1228.67, found [M+H]⁺: 1229.5.

### Example 34: Compound 34

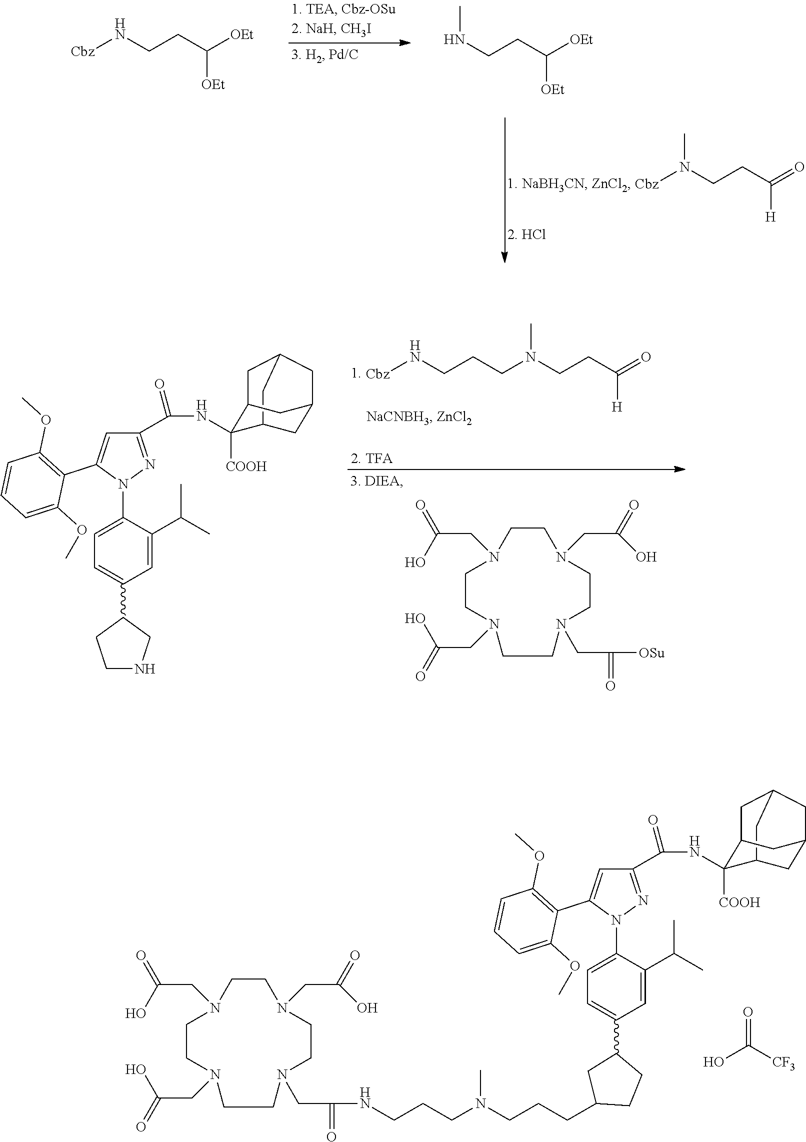

Step 1: Into a 250-mL round bottom flask, was placed a mixture of 3,3-diethoxypropan-1-amine (10 g, 1 Eq, 68 mmol), TEA (21 g, 29 mL, 3.1 Eq, 0.21 mol), benzyl (2,5-dioxopyrrolidin-1-yl) carbonate (19 g, 1.1 Eq, 76 mmol) and DCM (100 mL). The reaction mixture was stirred at 25° C. for 1 hour. The mixture was diluted with water (100 mL), extracted with EtOAc (50 mL×3), the combined organic layers were washed with water (50 mL×2), brine (50 mL), dried over anhydrous Na₂SO₄ and concentrated under reduced pressure. The crude product was purified by MPLC to provide benzyl (3,3-diethoxypropyl)carbamate (11 g, 39 mmol, 58%) as a colorless oil.

Step 2: Into a 250-mL round bottom flask, was placed a mixture of benzyl (3,3-diethoxypropyl)carbamate (11 g, 1 Eq, 39 mmol), sodium hydride (1.0 g, 1.1 Eq, 42 mmol) and DMF (100 mL). The reaction mixture was stirred at 0° C. for 30 minutes, then iodomethane (6.1 g, 1.1 Eq, 43 mmol) was added and the reaction mixture was stirred at 25° C. for an additional 1 hour. The mixture was diluted with water (500 mL), extracted with EtOAc (200 mL×3), the combined organic layers were washed with water (200 mL×2), brine (200 mL), dried over anhydrous Na₂SO₄ and concentrated under reduced pressure. The crude product was purified by MPLC to provide benzyl (3,3-diethoxypropyl)(methyl)carbamate (8 g, 0.03 mol, 70%) as a yellow oil. [M+Na]⁺=318.1

Steps 3-4: Benzyl (3,3-diethoxypropyl)(methyl)carbamate was treated with hydrogen and Pd/C in a manner similar to Example 1, Step 7 then treated with benzyl (3-oxopropyl)carbamate, ZnCl₂, and NaBH₃CN in a manner similar to Example 19, Step 1 to provide benzyl (3-((3,3-diethoxypropyl)(methyl)amino)propyl)carbamate (1.8 g, 5.1 mmol, 90%) as a yellow oil. [M+H]⁺=353.3.

Step 5: Into an 8-mL vial, was placed a mixture of benzyl (3-((3,3-diethoxypropyl)(methyl)amino)propyl)carbamate (700 mg, 1 Eq, 1.99 mmol) and IN HC (10 mL), the reaction mixture was stirred at 25° C. for 1 hour. The mixture was concentrated under reduced pressure to provide benzyl (3-(methyl(3-oxopropyl)amino)propyl)carbamate (400 mg, 1.44 mmol, 72.4%) as a colorless oil which was in the next step without further purification.

Step 6: Compound 4 (a single isomer of (1R,3S,5R,7R)-2-(5-(2,6-dimethoxyphenyl)-1-(2-isopropyl-4-(-pyrrolidin-3-yl)phenyl)-1H-pyrazole-3-carboxamido)adamantane-2-carboxylic acid), was treated with benzyl (3-(methyl(3-oxopropyl)amino)propyl)carbamate, ZnCl₂, and NaBH₃CN in a manner similar to Example 19, Step 1 to provide a single isomer of (1R,3 S,5R,7R)-2-(1-(4-(1-(3-((3-(((benzyloxy)carbonyl)amino)propyl)(methyl)amino)propyl)pyrrolidin-3-yl)-2-isopropylphenyl)-5-(2,6-dimethoxyphenyl)-11H-pyrazole-3-carboxamido)adamantane-2-carboxylic acid (20 mug, 23 μmol, 50%) as a yellow oil. [M+H]⁺=875.7.

Steps 7-8: The product from Step 6 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of a single isomer of 2,2′,2″-(10-(2-((3-((3-(−3-(4-(3-(((1R,3S,5R,7R)-2-carboxyadamantan-2-yl)carbamoyl)-5-(2,6-dimethoxyphenyl)-1H-pyrazol-1-yl)-3-isopropylphenyl)pyrrolidin-1-yl)propyl)(methyl)amino)propyl)amino)-2-oxoethyl)-1,4,7,10-tetraazacyclododecane-1,4,7-triyl)triacetic acid (21.6 mg, 17.4 μmol, 72%) as a white solid. MS: Calc'd for C₉H₈₆N₁₀O₁₂: 1126.64, found [M+H]⁺: 1127.7.

### Example 35: Compound 35

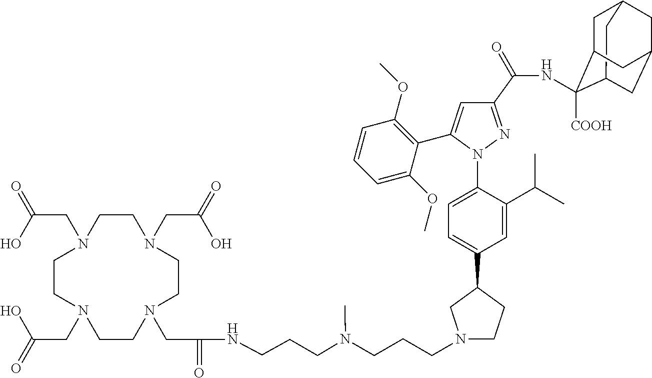 [SMILES: COc1cccc(OC)c1-c1cc(C(=O)NC2(C(=O)O)C3CC4CC(C3)CC2C4)nn1-c1ccc([C@H]2CCN(CCCN(C)CCCNC(=O)CN3CCN(CC(=O)O)CCN(CC(=O)O)CCN(CC(=O)O)CC3)C2)cc1C(C)C]

Steps 1-3: Compound 35 was prepared from Compound 5 (a single isomer of (1R,3S,5R,7R)-2-(5-(2,6-dimethoxyphenyl)-1-(2-isopropyl-4-(-pyrrolidin-3-yl)phenyl)-1H-pyrazole-3-carboxamido)adamantane-2-carboxylic acid) in a manner similar to Example 34, Steps 6-8 to provide a single isomer of 2,2′,2″-(10-(2-((3-((3-(−3-(4-(3-(((1R,3S,5R,7R)-2-carboxyadamantan-2-yl)carbamoyl)-5-(2,6-dimethoxyphenyl)-1H-pyrazol-1-yl)-3-isopropylphenyl)pyrrolidin-1-yl)propyl)(methyl)amino)propyl)amino)-2-oxoethyl)-1,4,7,10-tetraazacyclododecane-1,4,7-triyl)triacetic acid (26.1 mg, 21.0 μmol, 71%) as a white solid. MS: Calc'd for C₅₉H₈₆N₁₀O₁₂: 1126.64, found [M+H]⁺1127.7.

### Example 36: Compound 36

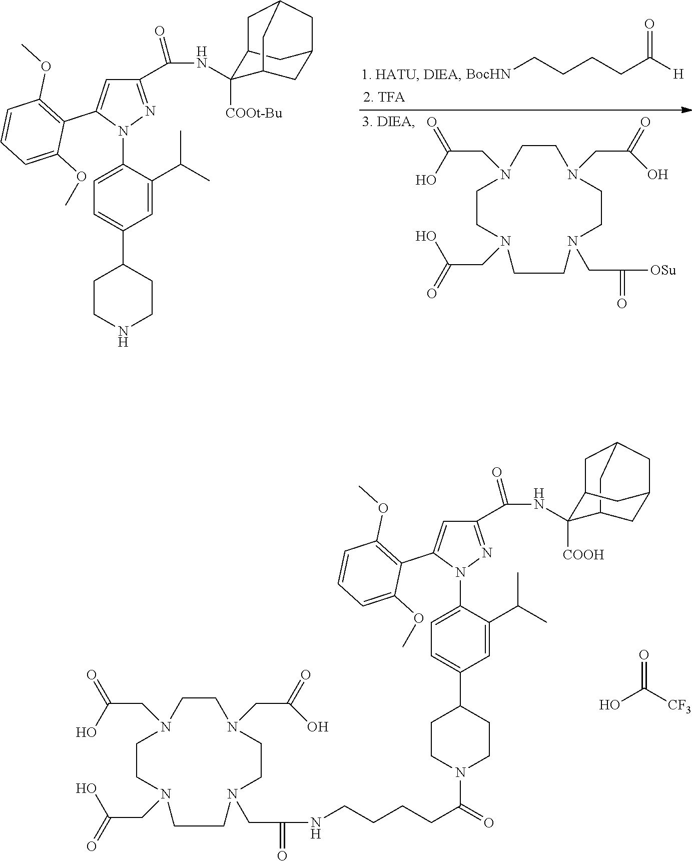

Step 1: tert-butyl (1R,3 S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate was treated with 5-((tert-butoxycarbonyl)amino)pentanoic acid, DIEA, and HATU to provide tert-butyl (1R,3S,5r,7r)-2-(1-(4-(1-(5-((tert-butoxy carbonyl)amino)pentanoyl)piperidin-4-yl)-2-isopropylphenyl)-5-(2,6-dimethoxyphenyl)-1H-pyrazole-3-carboxamido)adamantane-2-carboxylate (90% Purity) as a white solid. [M+H]⁺=882.7.

Steps 2-3: The product from Step 1 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((5-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-5-oxopentyl)amino)-2-oxoethyl)-1,4,7,10-tetraazacyclododecane-1,4,7-triyl)triacetic acid (17.4 mg, 15 kmol, 22%) as a white solid. MS: Calc'd for C₅₈H₈₁N₉O₁₃: 1111.6, found [M+H]⁺: 1112.6.

### Example 37: Compound 37

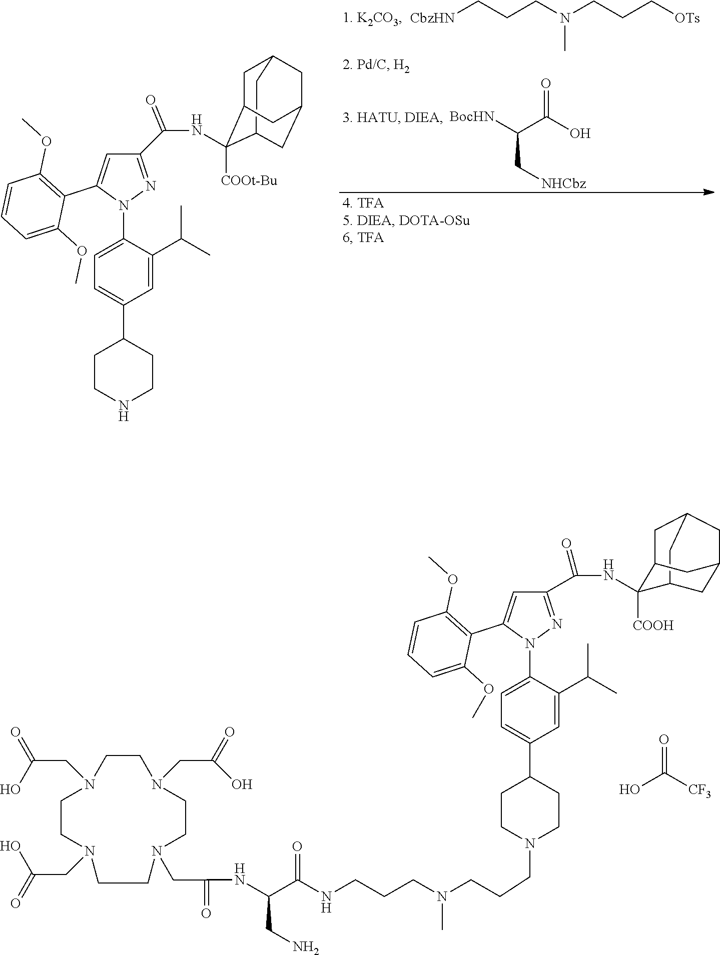

Step 1: To a mixture of benzyl (3-((3-hydroxypropyl)(methyl)amino)propyl)carbamate (6.0 g, 1 Eq, 21 mmol), K₂CO₃ (40.5 mg, 1 Eq, 293 μmol) and DMF (2 mL) was added 3-((3-(((benzyloxy)carbonyl)amino)propyl)(methyl)amino)propyl 4-methylbenzenesulfonate (127 mg, 1 Eq, 293 mol). The reaction mixture was stirred at 25° C. for 2 hour, then the mixture was directly purified by Prep-HPLC to provide (1R,3S,5r,7r)-2-(1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxamido)adamantane-2-carboxylic acid (230 mg, 0.20 mmol, 69%) as a white solid. [M+H⁺]=945.5.

Steps 2-3: (1R,3S,5r,7r)-2-(1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxamido)adamantane-2-carboxylic acid was treated with hydrogen and Pd/C in a manner similar to Example 1, Step 7, then was treated with HATU, DIEA, and (R)-3-(((benzyloxy)carbonyl)amino)-2-((tert-butoxycarbonyl)amino)propanoic acid in a manner similar to Example 1, Step 5 to provide tert-butyl (1R,3S,5r,7r)-2-(1-(4-(1-((R)-6-((tert-butoxycarbonyl)amino)-12-methyl-3,7-dioxo-1-phenyl-2-oxa-4,8,12-triazapentadecan-15-yl)piperidin-4-yl)-2-isopropylphenyl)-5-(2,6-dimethoxyphenyl)-1H-pyrazole-3-carboxamido)adamantane-2-carboxylate (70 mg, 60 μmol, 61%) as a white solid. [M+H⁺]=1131.7.

Steps 4-5: tert-Butyl (1R,3S,5r,7r)-2-(1-(4-(1-((R)-6-((tert-butoxycarbonyl)amino)-12-methyl-3,7-dioxo-1-phenyl-2-oxa-4,8,12-triazapentadecan-15-yl)piperidin-4-yl)-2-isopropylphenyl)-5-(2,6-dimethoxyphenyl)-1H-pyrazole-3-carboxamido)adamantane-2-carboxylate was treated with TFA, then 2,2′,2′-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-(((R)-15-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-12-methyl-3,7-dioxo-1-phenyl-2-oxa-4,8,12-triazapentadecan-6-yl)amino)-2-oxoethyl)-1,4,7,10-tetraazacyclododecane-1,4,7-triyl)triacetic acid (60 mg, 44 μmol, 54%) as a white solid. [M+H⁺]=1361.8.

Step 6: A mixture of 2,2′,2″-(10-(2-(((R)-15-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)-12-methyl-3,7-dioxo-1-phenyl-2-oxa-4,8,12-triazapentadecan-6-yl)amino)-2-oxoethyl)-1,4,7,10-tetraazacyclododecane-1,4,7-triyl)triacetic acid (60 mg, 1 Eq, 44 μmol) and TFA (1 mL) was stirred at 70° C. for 2 hours. The mixture was directly purified by Prep-HPLC to afford the 2,2,2-trifluoroacetic acid salt of 2,2′,2″-(1(0-(2-((R)-3-amino-1-((3-((3-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)(methyl)amino)propyl)amino)-1-oxopropan-2-yl)amino)-2-oxoethyl)-1,4,7,10-tetraazacyclododecane-1,4,7-triyl)triacetic acid as a white solid. MS: Calc'd for C₆₃H₉₄N₁₂O₁₃: 1226.71, found [M+H]⁺: 1227.7.

### Example 38: Compound 38

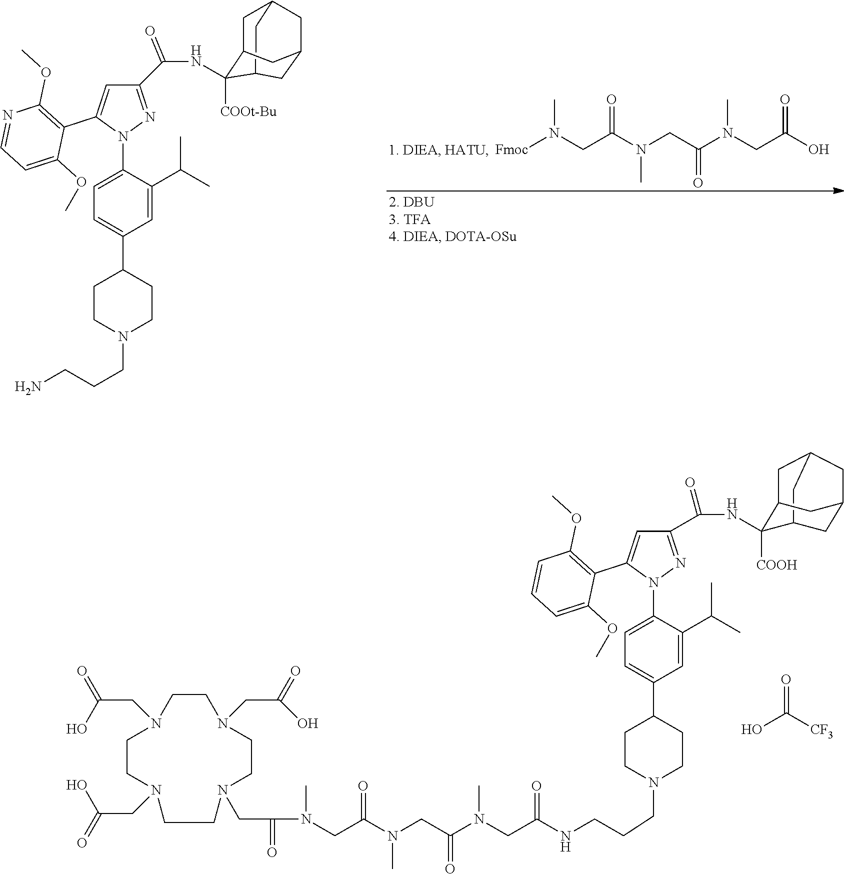

Step 1: tert-Butyl (1R,3S,5r,7r)-2-(1-(4-(1-(3-aminopropyl)piperidin-4-yl)-2-isopropylphenyl)-5-(2,6-dimethoxyphenyl)-1H-pyrazole-3-carboxamido)adamantane-2-carboxylate (85 mg, 1 Eq, 0.11 mmol) and 1-(9H-fluoren-9-yl)-4,7,10-trimethyl-3,6,9-trioxo-2-oxa-4,7,10-triazadodecan-12-oic acid (63 mg, 1.2 Eq, 0.14 mmol) were treated with DIEA and HATU in a manner similar to Example 1, Step 5 to provide tert-butyl (1R,3S,5r,7r)-2-(1-(4-(1-(1-(9H-fluoren-9-yl)-4,7,10-trimethyl-3,6,9,12-tetraoxo-2-oxa-4,7,10,13-tetraazahexadecan-16-yl)piperidin-4-yl)-2-isopropylphenyl)-5-(2,6-dimethoxyphenyl)-1H-pyrazole-3-carboxamido)adamantane-2-carboxylate (35 mg, 30 mol, 26%) as a yellow oil.

Step 2: To a mixture of tert-butyl (1R,3S,5r,7r)-2-(1-(4-(1-(1-(9H-fluoren-9-yl)-4,7,10-trimethyl-3,6,9,12-tetraoxo-2-oxa-4,7,10,13-tetraazahexadecan-16-yl)piperidin-4-yl)-2-isopropylphenyl)-5-(2,6-dimethoxyphenyl)-1H-pyrazole-3-carboxamido)adamantane-2-carboxylate (30 mg, 1 Eq, 26 μmol) and DIF (2 mL), was added DBU (0.1 mL) and the reaction mixture was stirred at 25° C. for 1 hour. The mixture was concentrated under reduced pressure. The crude product was purified by Prep-HPLC to afford tert-butyl (1R,3S,5r,7r)-2-(5-(2,6-dimethoxyphenyl)-1-(4-(1-(5,8-dimethyl-4,7,10-trioxo-2,5,8,11-tetraazatetradecan-14-yl)piperidin-4-yl)-2-isopropylphenyl)-1H-pyrazole-3-carboxamido)adamantane-2-carboxylate (25 mg, 26 μmol, 100%) as a white solid.

Steps 3-4: tert-Butyl (1R,3S,5r,7r)-2-(5-(2,6-dimethoxyphenyl)-1-(4-(1-(5,8-dimethyl-4,7,10-trioxo-2,5,8,11-tetraazatetradecan-14-yl)piperidin-4-yl)-2-isopropylphenyl)-1H-pyrazole-3-carboxamido)adamantane-2-carboxylate was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(15-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)-3,6,9-trimethyl-2,5,8,11-tetraoxo-3,6,9,12-tetraazapentadecyl)-1,4,7,10-tetraazacyclododecane-1,4,7-triyl)triacetic acid (7 mg, 5 μmol, 20%) as a white solid. MS: Calc'd for C₆₅1H₉₄N₁₂O₁₅: 1282.70, found [M+H]⁺: 1283.7.

### Example 39: Compound 39

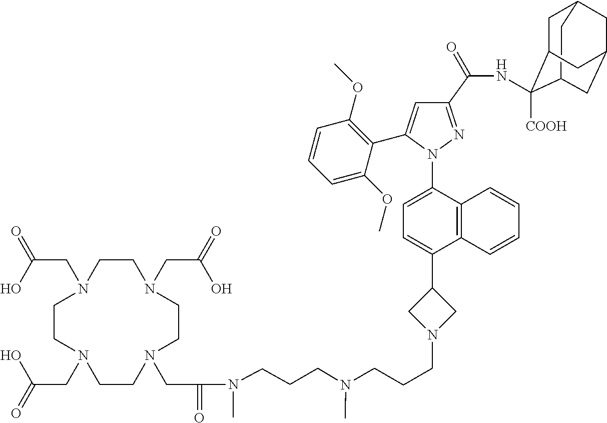 [SMILES: COc1cccc(OC)c1-c1cc(C(=O)NC2(C(=O)O)C3CC4CC(C3)CC2C4)nn1-c1ccc(C2CN(CCCN(C)CCCN(C)C(=O)CN3CCN(CC(=O)O)CCN(CC(=O)O)CCN(CC(=O)O)CC3)C2)c2ccccc12]

### Example 40: Compound 40

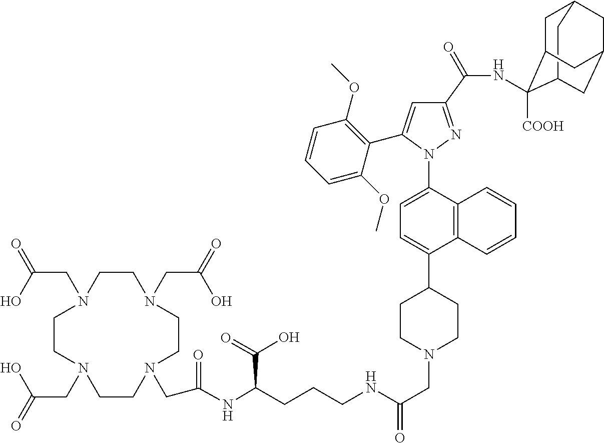 [SMILES: COc1cccc(OC)c1-c1cc(C(=O)NC2(C(=O)O)C3CC4CC(C3)CC2C4)nn1-c1ccc(C2CCN(CC(=O)NCCC[C@@H](NC(=O)CN3CCN(CC(=O)O)CCN(CC(=O)O)CCN(CC(=O)O)CC3)C(=O)O)CC2)c2ccccc12]

### Example 41: Compound 41

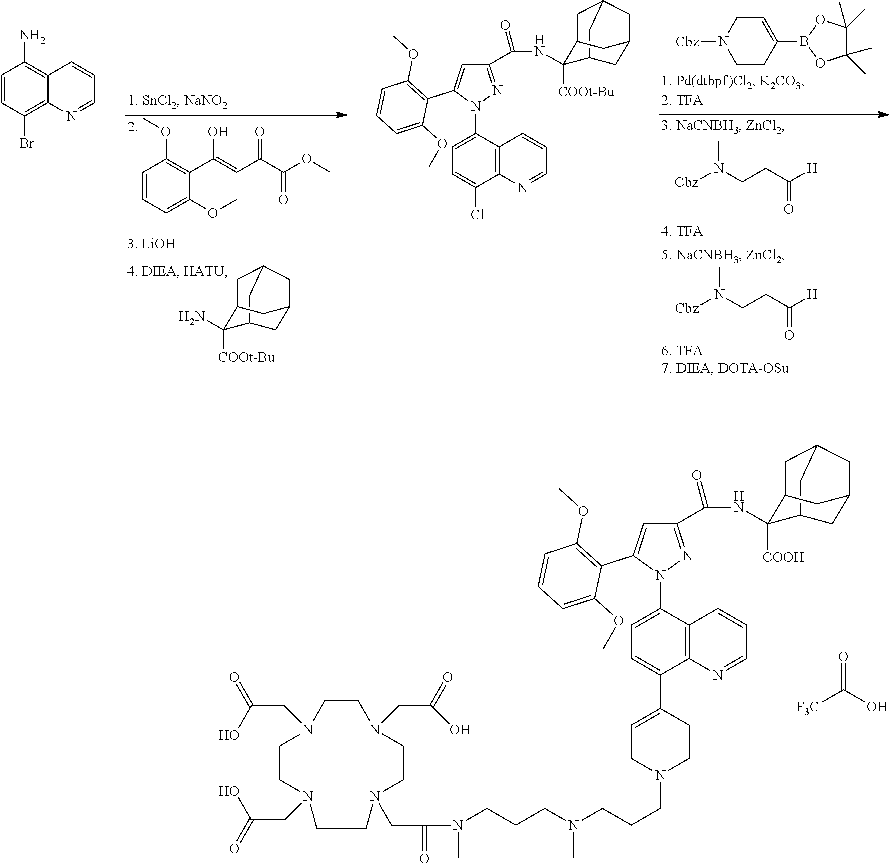

Steps 1-4: tert-Butyl (1R,3 S,5r,7r)-2-(1-(8-chloroquinolin-5-yl)-5-(2,6-dimethoxyphenyl)-1H-pyrazole-3-carboxamido)adamantane-2-carboxylate was prepared from 8-bromoquinolin-5-amine in a manner similar to Example 8, Steps 2-5 to afford the product (560 mg, 871 μmol, 24.6%) as a yellow oil.

Step 5: To a mixture of tert-butyl (1R,3S,5r,7r)-2-(1-(8-chloroquinolin-5-yl)-5-(2,6-dimethoxyphenyl)-1H-pyrazole-3-carboxamido)adamantane-2-carboxylate (450 mg, 1 Eq, 700 μmol), Pd(dtbpf)Cl₂ (45.6 mg, 0.100 Eq, 70.0 μmol), benzyl 4-(4,4,5,5-tetramethyl-1,3,2-dioxaborolan-2-yl)-3,6-dihydropyridine-1(2H)-carboxylate (360 mg, 1.50 Eq, 1.05 mmol), K₂CO₃ (290 mg, 3.00 Eq, 2.10 mmol) and 1,4-dioxane (1.4 mL) was added water (0.2 mL). The reaction mixture was stirred at 90° C. 1 hour under N₂. The mixture was directly purified by MPLC to afford benzyl 4-(5-(3-(((1R,3S,5r,7r)-2-(tert-butoxycarbonyl)adamantan-2-yl)carbamoyl)-5-(2,6-dimethoxyphenyl)-1H-pyrazol-1-yl)quinolin-8-yl)-3,6-dihydropyridine-1(2H)-carboxylate (380 mg, 461 μmol, 65.9%) as a yellow solid. [M+H]⁺=824.5.

Step 6: A mixture of benzyl 4-(5-(3-(((1R,3S,5r,7r)-2-(tert-butoxycarbonyl)adamantan-2-yl)carbamoyl)-5-(2,6-dimethoxyphenyl)-1H-pyrazol-1-yl)quinolin-8-yl)-3,6-dihydropyridine-1(2H)-carboxylate (380 mg, 1 Eq, 461 μmol) and TFA (3 mL) was stirred at 60° C. for 1 hour. The mixture was directly purified by MPLC to afford (1R,3S,5r,7r)-2-(5-(2,6-dimethoxyphenyl)-1-(8-(1,2,3,6-tetrahydropyridin-4-yl)quinolin-5-yl)-1H-pyrazole-3-carboxamido)adamantane-2-carboxylic acid (300 mg, 473 μmol, 103%) as a yellow oil. [M+H]⁺=634.3.

Step 7: A mixture of (1R,3S,5r,7r)-2-(5-(2,6-dimethoxyphenyl)-1-(8-(1,2,3,6-tetrahydropyridin-4-yl)quinolin-5-yl)-1H-pyrazole-3-carboxamido)adamantane-2-carboxylic acid (155 mg, 1 Eq, 245 μmol), benzyl methyl(3-oxopropyl)carbamate (135 mg, 2.49 Eq, 610 μmol), zinc chloride (66.7 mg, 30.9 μL, 2.00 Eq, 489 μmol) and MeOH (2 mL) was stirred at 20° C. for 10 minutes, then NaCNBH₃ (30.7 mg, 2.00 Eq, 489 μmol) was added and the reaction mixture was stirred at 25° C. for an additional 1 hour. The mixture was directly purified by MPLC to afford (1R,3S,5r,7r)-2-(1-(8-(1-(3-(((benzyloxy)carbonyl)(methyl)amino)propyl)-1,2,3,6-tetrahydropyridin-4-yl)quinolin-5-yl)-5-(2,6-dimethoxyphenyl)-1H-pyrazole-3-carboxamido)adamantane-2-carboxylic acid (100 mg, 119 μmol, 48.7%) as an off-white oil. [M+H]⁺=839.4.

Steps 8-9: The product from Step 7 was treated with TFA then benzyl methyl(3-oxopropyl)carbamate in a manner similar to Steps 6 and 7 to provide (1R,3S,5r,7r)-2-(1-(8-(1-(3-((3-(((benzyloxy)carbonyl)(methyl)amino)propyl)(methyl)amino)propyl)-1,2,3,6-tetrahydropyridin-4-yl)quinolin-5-yl)-5-(2,6-dimethoxyphenyl)-1H-pyrazole-3-carboxamido)adamantane-2-carboxylic acid (40 mg, 44 μmol, 39%) as an off-white oil. [M+H]⁺=910.6.

Steps 10-11: The product from Step 9 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((3-(4-(5-(5-(2,6-dimethoxyphenyl)-3-(((1R,3S,5r,7r)-2-(hydroxy(oxo)-15-methyl)adamantan-2-yl)carbamoyl)-1H₁-pyrazol-1-yl)quinolin-8-yl)-3,6-dihydropyridin-1(2H)-yl)propyl)(methyl)amino)propyl)(methyl)amino)-2-oxoethyl)-1,4,7,10-tetraazacyclododecane-1,4,7-triyl)triacetic acid (5.4 mg, 4.2 μmol, 11%) as a white solid. MS: Calc'd for C₁H₈₄N₁₁O₁₂: 1162.63, found [M+H]⁺: 1162.5.

### Example 42: Compound 42

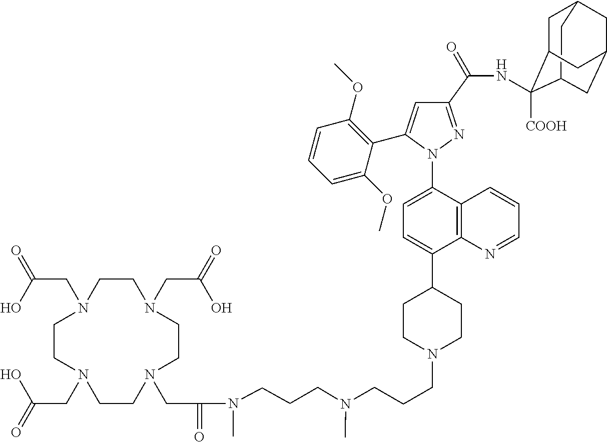 [SMILES: COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)c2ncccc12]

### Example 43: Compound 43

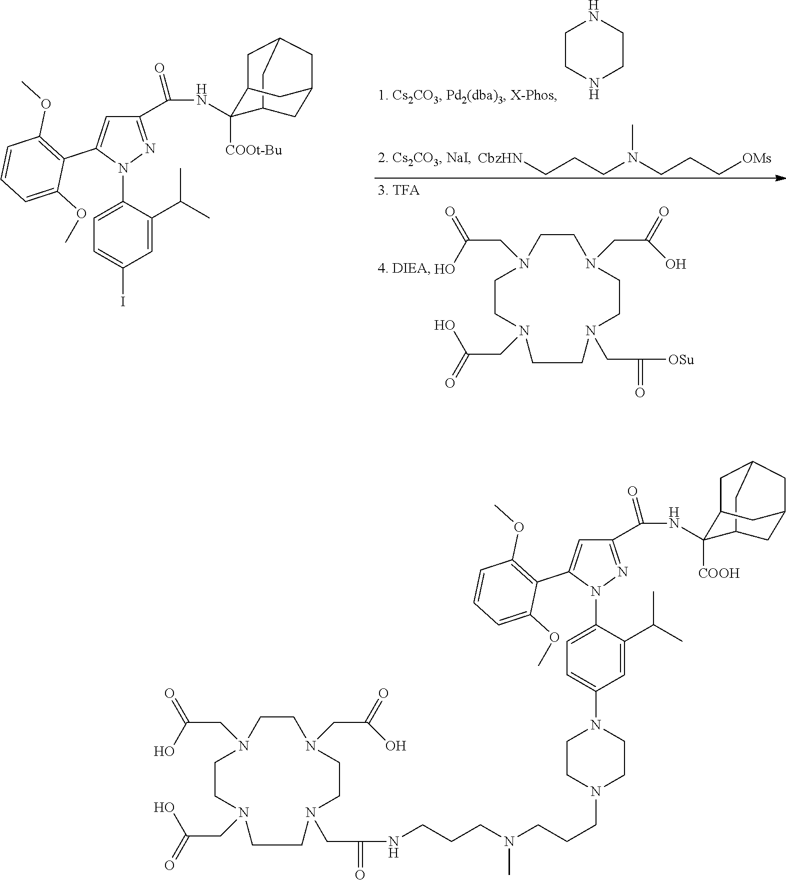

Step 1: Into a 40-mL vial under an atmosphere of nitrogen, was placed a mixture of tert-butyl (1R,3 S,5r,7r)-2-(5-(2,6-dimethoxyphenyl)-1-(4-iodo-2-isopropylphenyl)-1H-pyrazole-3-carboxamido)adamantane-2-carboxylate (700 mg, 1 Eq, 965 μmol), piperazine (332 mg, 4.00 Eq. 3.85 mmol), Cs₂CO₃ (943 mg, 3.00 Eq, 2.89 mmol), Pd₂(dba)₃ (88.3 mg, 0.100 Eq, 96.4 μmol), X-phos (92.0 mg, 0.200 Eq, 193 μmol), and dioxane (10 mL). The reaction mixture was stirred at 120° C. for 2 hours, then the mixture was concentrated and the crude product was purified by MPLC to provide tert-butyl (1R,3S,5r,7r)-2-(5-(2,6-dimethoxyphenyl)-1-(2-isopropyl-4-(piperazin-1-yl)phenyl)-1H-pyrazole-3-carboxamido)adamantane-2-carboxylate (490 mg, 716 μmol, 74.3%) as a yellow oil. [M+H]⁺=684.9.

Step 2: tert-Butyl (1R,3S,5r,7r)-2-(5-(2,6-dimethoxyphenyl)-1-(2-isopropyl-4-(piperazin-1-yl)phenyl)-1H-pyrazole-3-carboxamido)adamantane-2-carboxylate was treated with Cs₂CO₃, sodium iodide, and 3-((3-(((benzyloxy)carbonyl)amino)propyl)(methyl)amino)propyl methanesulfonate in a manner similar to Example 13, Step 3 to provide tert-butyl (1R,3 S,5r,7r)-2-(1-(4-(4-(3-((3-(((benzyloxy)carbonyl)amino)propyl)(methyl)amino)propyl)piperazin-1-yl)-2-isopropylphenyl)-5-(2,6-dimethoxyphenyl)-1H-pyrazole-3-carboxamido)adamantane-2-carboxylate (62 mg, 66 μmol, 56%) as a yellow oil. [M+H]⁺=946.9.

Steps 3-4: (1R,3 S,5r,7r)-2-(1-(4-(4-(3-((3-aminopropyl)(methyl)amino)propyl)piperazin-1-yl)-2-isopropylphenyl)-5-(2,6-dimethoxyphenyl)-1H-pyrazole-3-carboxamido)adamantane-2-carboxylic acid was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperazin-1-yl)propyl)(methyl)amino)propyl)amino)-2-oxoethyl)-1,4,7,10-tetraazacyclododecane-1,4,7-triyl)triacetic acid (20.8 mg, 16.6 μmol, 25%) as a white solid. MS: Calc'd for C₅₉H₈₇N₁₁O₁₂: 1141.65, found [M+H]⁺: 1142.6.

### Example 44: Compound 44

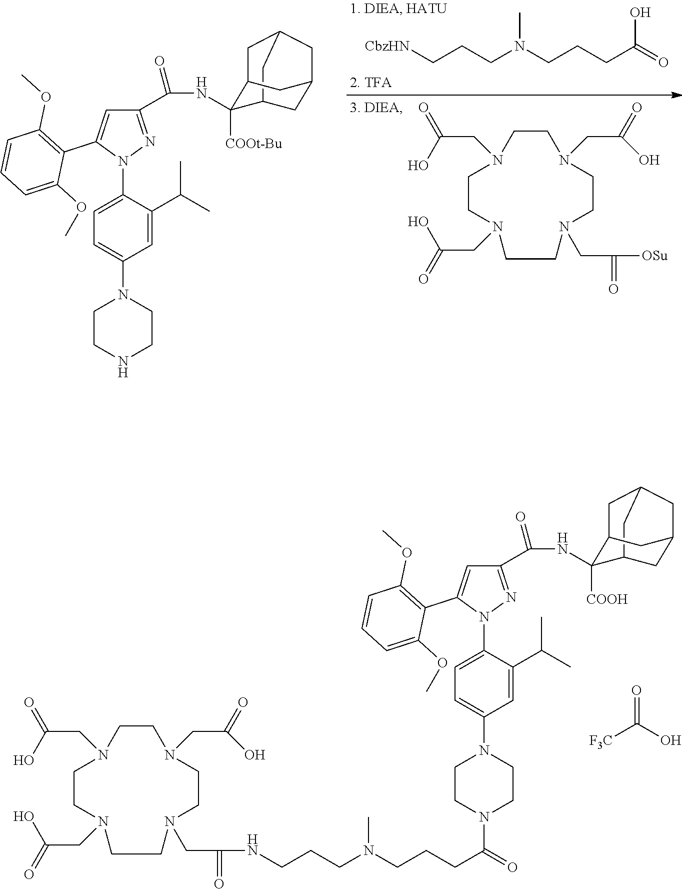

Compound 44 was prepared from 4-((3-(((benzyloxy)carbonyl)amino)propyl)(methyl)amino)butanoic acid and tert-butyl (1R,3 S,5r,7r)-2-(5-(2,6-dimethoxyphenyl)-1-(2-isopropyl-4-(piperazin-1-yl)phenyl)-11H-pyrazole-3-carboxamido)adamantane-2-carboxylate in a manner similar to Example 14, Step 2, then Example 13, Steps 4 and 5 to provide the 2,2,2-trifluoroacetic acid salt of 2,2′,2″-(10-(2-((3-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperazin-1-yl)-4-oxobutyl)(methyl)amino)propyl)amino)-2-oxoethyl)-1,4,7,10-tetraazacyclododecane-1,4,7-triyl)triacetic acid (38.0 mg, 29.6 μmol, 39%) as a white solid. MS: Calc'd for C₆₀H₈₇N₁₁O₁₃: 1169.65, found [M+H]⁺: 1170.9.

### Example 45: Compound 45

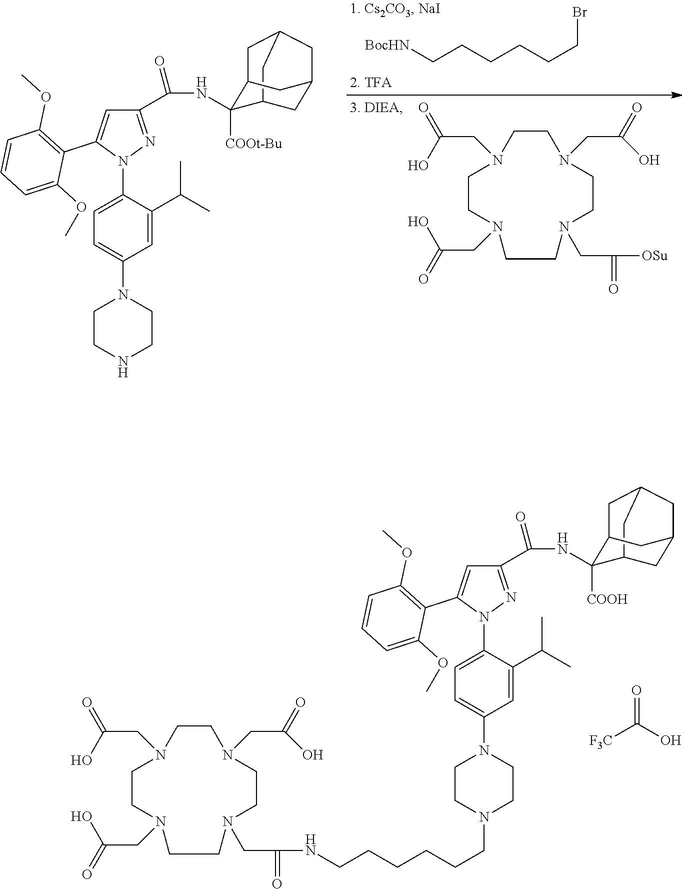

Compound 45 was prepared from tert-butyl (6-bromohexyl)carbamate and tert-butyl (1R,3S,5r,7r)-2-(5-(2,6-dimethoxyphenyl)-1-(2-isopropyl-4-(piperazin-1-yl)phenyl)-1H-pyrazole-3-carboxamido)adamantane-2-carboxylate in a manner similar to Example 12, Step 3, then Example 13, Steps 4 and 5 to provide the 2,2,2-trifluoroacetic acid salt of 2,2′,2″-(10-(2-((6-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperazin-1-yl)hexyl)amino)-2-oxoethyl)-1,4,7,10-tetraazacyclododecane-1,4,7-triyl)triacetic acid (36.9 mg, 30.1 μmol, 34%) as a white solid. MS: Calc'd for C₅₈H₈₄N₁₁O₁₂: 1112.63, found [M+H]⁺: 1114.0.

### Example 46: Compound 46

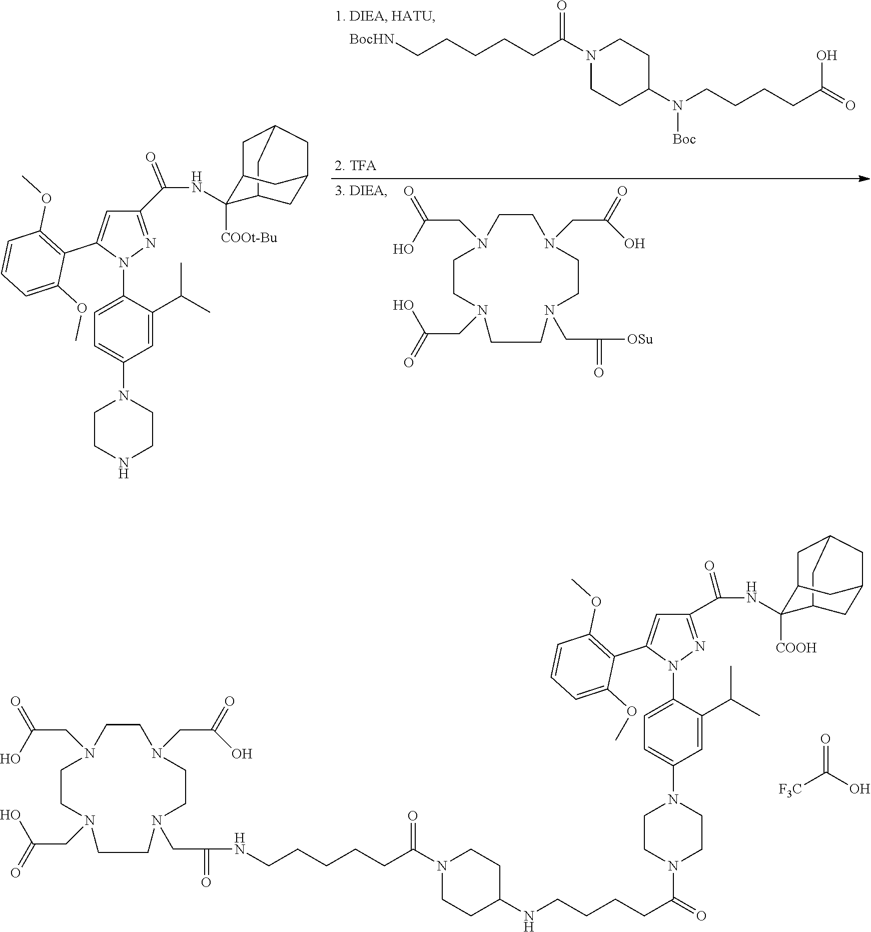

Compound 46 was prepared from 5-((tert-butoxycarbonyl)(1-(6-((tert-butoxycarbonyl)amino)hexanoyl)piperidin-4-yl)amino)pentanoic acid and tert-butyl (1R,3 S,5r,7r)-2-(5-(2,6-dimethoxyphenyl)-1-(2-isopropyl-4-(piperazin-1-yl)phenyl)-1H-pyrazole-3-carboxamido)adamantane-2-carboxylate in a manner similar to Example 14, Step 2, then Example 13, Steps 4 and 5 to provide the 2,2,2-trifluoroacetic acid salt of 2,2′,2″-(10-(2-((6-(4-((5-(4-(4-(³)-(((1R,3S,5r,7r)-2-carboxyadamantan-2-yl)carbamoyl)-5-(2,6-dimethoxy phenyl)-1H-pyrazol-1-yl)-3-isopropylphenyl)piperazin-1-yl)-5-oxopentyl)amino)piperidin-1-yl)-6-oxohexyl)amino)-2-oxoethyl)-1,4,7,10-tetraazacyclododecane-1,4,7-triyl)triacetic acid (46.5 mg, 32.7 μmol, 60%) as a white solid. MS: Calc'd for C₆₈H₁₀₀N₁₂O₁₄: 1308.75, found [M+H]⁺: 1310.2.

### Example 47: Compound 47

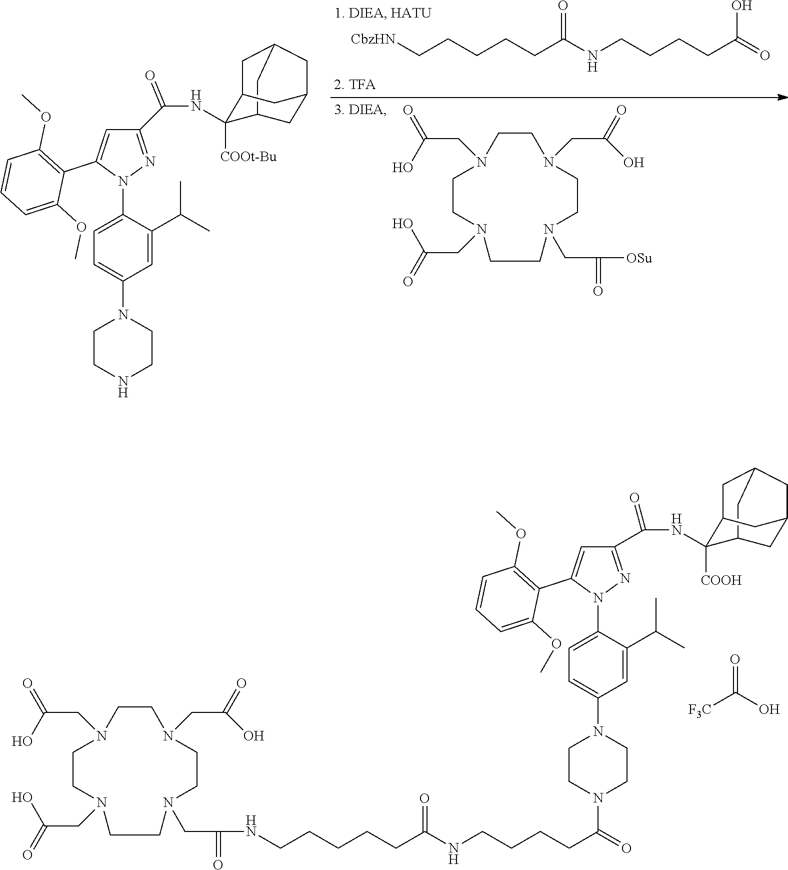

Compound 46 was prepared from 5-(6-(((benzyloxy)carbonyl)amino)hexanamido)pentanoic acid and tert-butyl (1R,3S,5r,7r)-2-(5-(2,6-dimethoxyphenyl)-1-(2-isopropyl-4-(piperazin-1-yl)phenyl)-1H-pyrazole-3-carboxamido)adamantane-2-carboxylate in a manner similar to Example 14, Step 2, then Example 13, Steps 4 and 5 to provide the 2,2,2-trifluoroacetic acid salt of 2,2′,2″-(10-(2-((6-((5-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperazin-1-yl)-5-oxopentyl)amino)-6-oxohexyl)amino)-2-oxoethyl)-1,4,7,10-tetraazacyclododecane-1,4,7-triyl)triacetic acid (44.6 mg, 33.3 μmol, 51%) as a white solid. MS: Calc'd for C₆₃H₉₁N₁₁O₁₄: 1225.67, found [M+H]⁺: 1227.0.

### Example 48: Compound 48

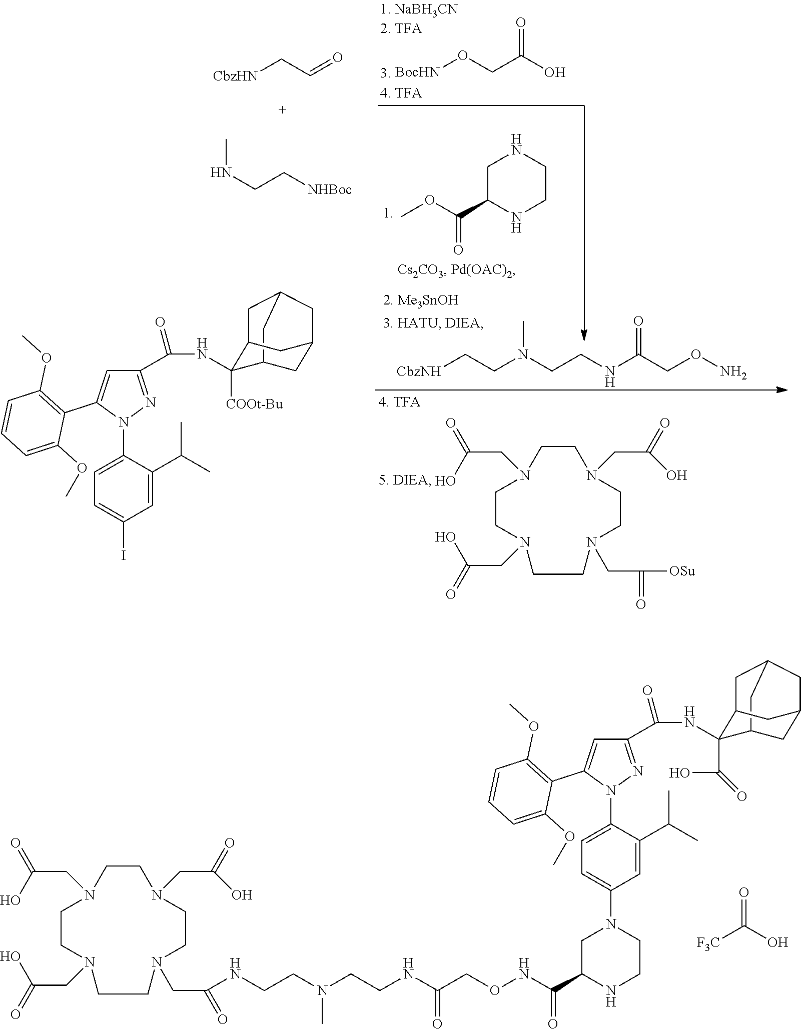

Step 1: Into a 50-mL vial, was placed a mixture of benzyl (2-oxoethyl)carbamate (1 g, 1 Eq, 5 mmol) and tert-butyl (2-(methylamino)ethyl)carbamate (0.9 g, 1 Eq, 5 mmol) in MeOH (10 mL). The reaction mixture was stirred at 25° C. for 30 minutes, then sodium cyanotrihydroborate (1.0 g, 3 Eq, 16 mmol) was added into the reaction mixture and stirred at 25° C. for 1 hour. The mixture was concentrated under reduced pressure then directly purified by MPLC to provide tert-butyl (2-((2-(((benzyloxy)carbonyl)amino)ethyl)(methyl)amino)ethyl)carbamate (400 mg, 1.14 mmol, 20%) as a white solid. [M+H]⁺=352.2.

Step 2: The product from Step 2 was treated with TFA in a manner similar to Example 13, Step 4 to provide crude benzyl (2-((2-aminoethyl)(methyl)amino)ethyl)carbamate which was used directly in the next step without purification.

Step 3: To a mixture of benzyl (2-((2-aminoethyl)(methyl)amino)ethyl)carbamate (400 mg, 1 Eq, 1.59 mmol) and 2-(((tert-butoxycarbonyl)amino)oxy)acetic acid (456 mg, 1.50 Eq, 2.39 mmol) in DMF (1 mL), was added propylphosphonic anhydride (1.01 g, 934 μL, 1.99 Eq, 3.17 mmol). The resulting mixture was stirred at 25° C. for 1 hour. The mixture was directly purified by MPLC to provide tert-butyl ((7-methyl-3,11-dioxo-1-phenyl-2-oxa-4,7,10-triazadodecan-12-yl)oxy)carbamate (150 mg, 353 μmol, 22.2%) as a white solid.

Step 4: The product from Step 2 was treated with TFA in a manner similar to Example 13, Step 4 to provide crude benzyl (2-((2-(2-(aminooxy)acetamido)ethyl)(methyl)amino)ethyl)carbamate which was used directly in the next step without purification.

Step 5: Into a 40-mL vial under an inert atmosphere of nitrogen, was placed a mixture of tert-butyl (1R,3 S,5r,7r)-2-(5-(2,6-dimethoxyphenyl)-1-(4-iodo-2-isopropylphenyl)-1H-pyrazole-3-carboxamido)adamantane-2-carboxylate (210 mg, 1 Eq, 289 mol), 1-(tert-butyl) 2-methyl (R)-piperazine-1,2-dicarboxylate (106 mg, 1.5 Eq, 434 μmol), palladium(II) acetate (13.0 mg, 0.2 Eq, 57.9 μmol), cesium carbonate (283 mg, 3 Eq, 868 μmol), dicyclohexyl(2′,6′-diisopropoxy-[1,1′-biphenyl]-2-yl)phosphane (27.0 mg, 0.2 Eq, 57.9 μmol) and 1,4-dioxane (1 mL). The reaction mixture was stirred at 100° C. for 3 hours under N₂. The mixture was directly purified by MPLC to afford 1-(tert-butyl) 2-methyl (R)-4-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-1,2-dicarboxylate (150 mg, 178 μmol, 61.6%) as a yellow solid. [M+H]=842.6

Step 6: Into an 8-mL round-bottom flask under an inert atmosphere of nitrogen, was placed 1-(tert-butyl) 2-methyl (R)-4-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-1,2-dicarboxylate (150 mg, 1 Eq, 178 μmol) and trimethylstannanol (161 mg, 5 Eq, 891 μmol), in DCE (1 mL) The mixture was stirred for 90° C. at 16 hours. The reaction mixture was filtered through a pad of celite, the filtrate was concentrated then purified by MPLC to provide (R)-1-(tert-butoxycarbonyl)-4-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-2-carboxylic acid (120 μg, 145 μmol, 81.4%) as a white solid.

Steps 7-9: The product from Step 6 was treated with benzyl (2-((2-(2-(aminooxy)acetamido)ethyl)(methyl)amino)ethyl)carbamate, HATU, and DIEA in a manner similar to Example 14, Step 2, then treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(1-((R)-4-(4-(3-(((1R,3S,5R,7R)-2-carboxyadamantan-2-yl)carbamoyl)-5-(2,6-dimethoxyphenyl)-1H-pyrazol-1-yl)-3-isopropylphenyl)piperazin-2-yl)-9-methyl-1,5,13-trioxo-3-oxa-2,6,9,12-tetraazatetradecan-14-yl)-1,4,7,10-tetraazacyclododecane-1,4,7-triyl)triacetic acid (5.5 mg, 4.1 μmol, 5.8%) as a white solid. MS: Calc'd for C₆₀H₈₇N₁₃O₁₅: 1229.64, found [M+H]⁺: 1230.6.

### Example 49: Compound 49

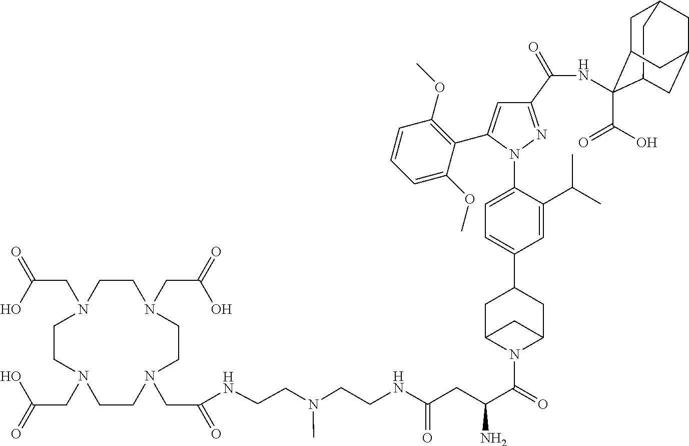 [SMILES: COc1cccc(OC)c1-c1cc(C(=O)NC2(C(=O)O)C3CC4CC(C3)CC2C4)nn1-c1ccc(C2CC3CC(C2)N3C(=O)[C@@H](N)CC(=O)NCCN(C)CCNC(=O)CN2CCN(CC(=O)O)CCN(CC(=O)O)CCN(CC(=O)O)CC2)cc1C(C)C]

### Example 50: Compound 50

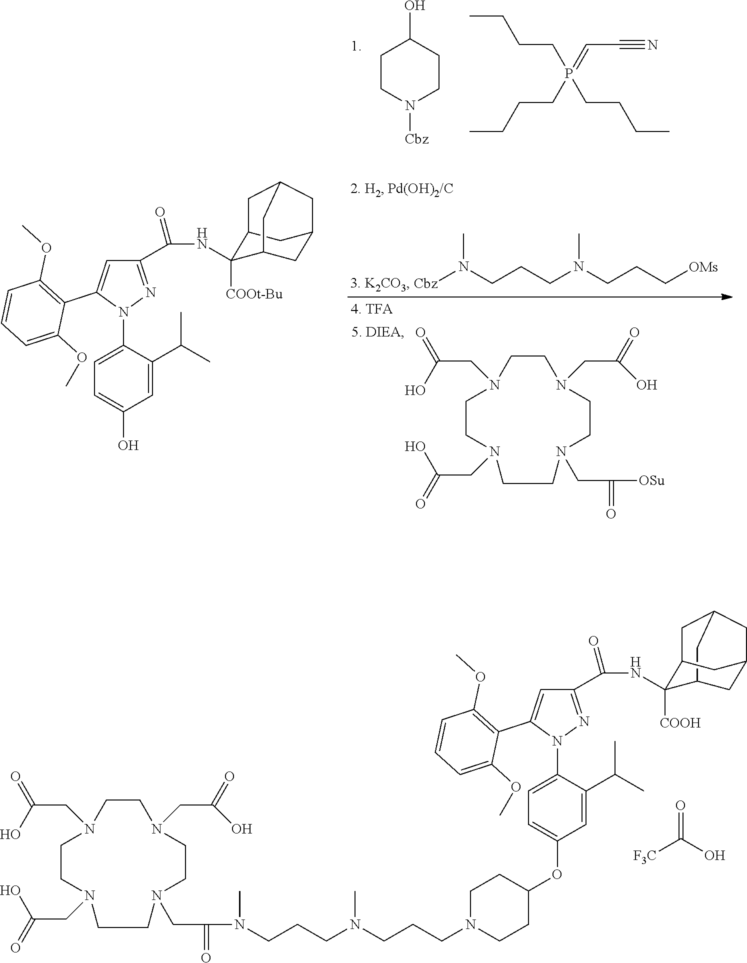

Step 1: Into a 40-mL vial, was placed a mixture of tert-butyl (1R,3 S,5r,7r)-2-(5-(2,6-dimethoxyphenyl)-1-(4-hydroxy-2-isopropylphenyl)-1H-pyrazole-3-carboxamido)adamantane-2-carboxylate (800 mg, 1 Eq, 1.30 mmol), benzyl 4-hydroxypiperidine-1-carboxylate (611 mg, 2.00 Eq, 2.60 mmol), cyanomethylenetributylphosphorane (627 mg, 688 μL, 2.00 Eq, 2.60 mmol) and toluene (20 mL). The reaction mixture was stirred at 110° C. for 2 hours. The reaction mixture was concentrated under reduced pressure to remove solvent, then the crude product was purified by MPLC to provide benzyl 4-(4-(3-(((1R,3 S,5r,7r)-2-(tert-butoxycarbonyl)adamantan-2-yl)carbamoyl)-5-(2,6-dimethoxyphenyl)-1H-pyrazol-1-yl)-3-isopropylphenoxy)piperidine-1-carboxylate (1.0 g, 1.2 mmol, 92%) as a white solid. [M+Na]⁺=855.5.

Step 2: Into a 50-mL round-bottom flask under an inert atmosphere of nitrogen, was placed benzyl 4-(4-(3-(((1R,3 S,5r,7r)-2-(tert-butoxy carbonyl)adamantan-2-yl)carbamoyl)-5-(2,6-dimethoxyphenyl)-1H-pyrazol-1-yl)-3-isopropylphenoxy)piperidine-1-carboxylate (1.0 g, 1 Eq, 1.2 mmol) and Pearlman's catalyst (400 mg, 3.1 Eq, 3.76 mmol), to which was carefully added 1PA (20 mL). The flask was evacuated and flushed with hydrogen three times, then the mixture was stirred for 1 hour at 25° C. The reaction mixture was filtered through a pad of celite, the filtrate was concentrated and purified by MPLC to provide tert-butyl (1R,3 S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (800 mg, 1.14 mmol, 95%) as a white solid. [M+H]⁺=698.7.

Step 3: The product from Step 2 was treated with 3-((3-(((benzyloxy)carbonyl)(methyl)amino)propyl)(methyl)amino)propyl methanesulfonate and K₂CO₃ in a manner similar to Example 13, Step 3 to provide tert-butyl (1R,3S,5r,7r)-2-(1-(4-((1-(3-((3-(((benzyloxy)carbonyl)(methyl)amino)propyl)(methyl)amino)propyl)piperidin-4-yl)oxy)-2-isopropylphenyl)-5-(2,6-dimethoxyphenyl)-1H-pyrazole-3-carboxamido)adamantane-2-carboxylate (50 mg, 51 μmol, 51%) as a yellow oil. [M+H]⁺: 975.6.

Steps 4-5: The product from Step 3 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((3-(4-(4-(3-(((1R,3S,5r,7r)-2-carboxyadamantan-2-yl)carbamoyl)-5-(2,6-dimethoxyphenyl)-11H-pyrazol-1-yl)-3-isopropylphenoxy)piperidin-1-yl)propyl)(methyl)amino)propyl)(methyl)amino)-2-oxoethyl)-1,4,7,10-tetraazacyclododecane-1,4,7-triyl)triacetic acid (27.8 mg, 21.6 μmol, 45%) as a white solid. MS: Calc'd for C₆₁H₉₀N₁₀O₁₃: 1170.67, found [M+H]⁺: 1171.8.

### Example 51: Compound 51

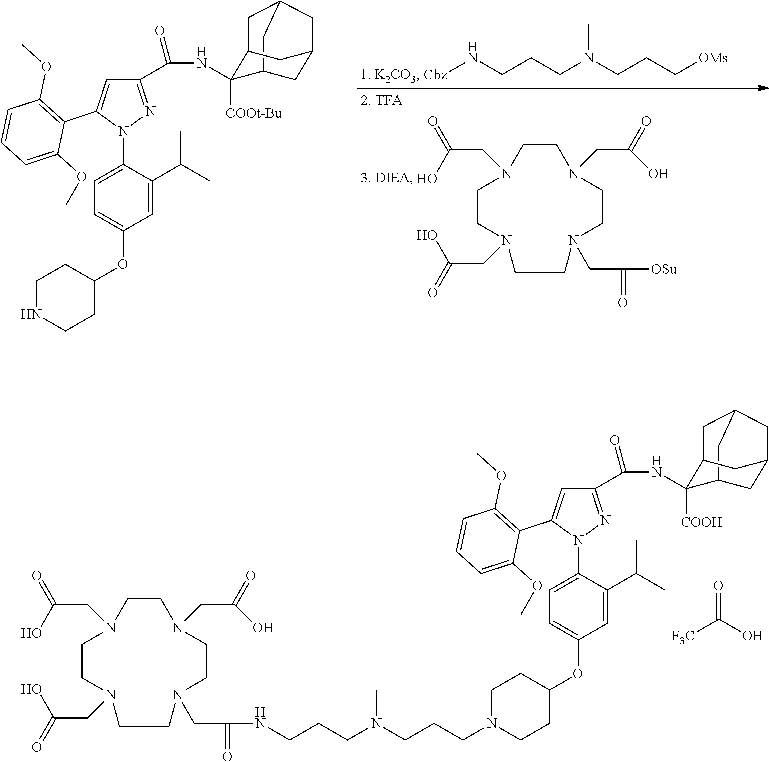

Compound 51 was synthesized from tert-butyl (1R,3S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (70 mg, 1 Eq, 0.10 mmol) and 3-((3-(((benzyloxy)carbonyl)amino)propyl)(methyl)amino)propyl methanesulfonate (72 mug, 2.0 Eq, 0.20 mmol) in manner similar to Example 50, Steps 3-5 to provide the trifluoroacetic acid salt of 2,2′,2″-(10-(2-((3-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenoxy)piperidin-1-yl)propyl)(methyl)amino)propyl)amino)-2-oxoethyl)-1,4,7,10-tetraazacyclododecane-1,4,7-triyl)triacetic acid (57.1 mg, 44.9 μmol, 87%) as a white solid. MS: Calc'd for C₆₀H₈₈N₁₀O₁₃: 1156.65, found [M+H]⁺: 1157.7.

### Example 52: Compound 52

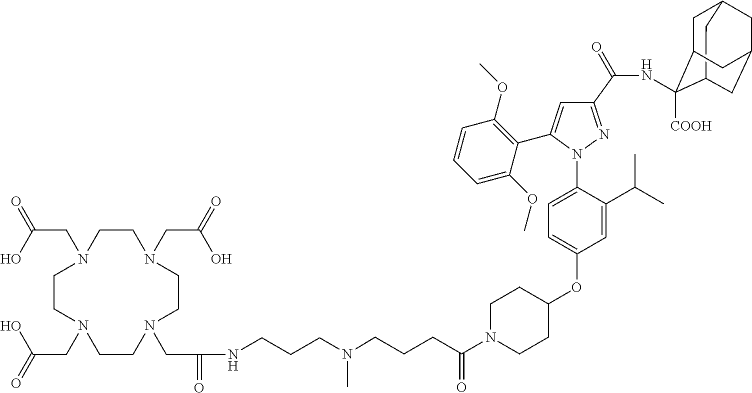 [SMILES: COc1cccc(OC)c1-c1cc(C(=O)NC2(C(=O)O)C3CC4CC(C3)CC2C4)nn1-c1ccc(OC2CCN(C(=O)CCCN(C)CCCNC(=O)CN3CCN(CC(=O)O)CCN(CC(=O)O)CCN(CC(=O)O)CC3)CC2)cc1C(C)C]

Compound 52 was synthesized from 4-((3-(((benzyloxy)carbonyl)amino)propyl)(methyl)amino)butanoic acid (46 mg, 1.5 Eq, 0.15 mmol) and tert-butyl (1R,3S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (70 mg, 1 Eq, 0.10 mmol) in manner similar to Example 14, Step 2 then Example 13, Steps 4 and 5 to provide the trifluoroacetic acid salt of 2,2′,2′-(10-(2-((3-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenoxy)piperidin-1-yl)-4-oxobutyl)(methyl)amino)propyl)amino)-2-oxoethyl)-1,4,7,10-tetraazacyclododecane-1,4,7-triyl)triacetic acid (57.1 mg, 43.9 μmol, 84%) as a white solid. MS: Calc'd for C₆₁H₈₈N₁₀O₁₄: 1184.65, found [M+H]⁺: 1185.8.

### Example 53: Compound 53

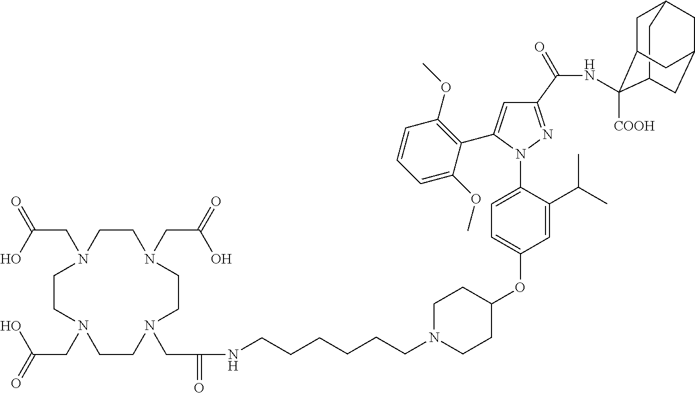 [SMILES: COc1cccc(OC)c1-c1cc(C(=O)NC2(C(=O)O)C3CC4CC(C3)CC2C4)nn1-c1ccc(OC2CCN(CCCCCCNC(=O)CN3CCN(CC(=O)O)CCN(CC(=O)O)CCN(CC(=O)O)CC3)CC2)cc1C(C)C]

Compound 52 was synthesized from tert-butyl (1R,3S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (60 mg, 1 Eq, 86 μmol) and tert-butyl (6-oxohexyl)carbamate (18 mg, 0.97 Eq, 84 μmol) in a manner similar to Example 19, Step 1 then Example 13, Steps 4 and 5 to provide the trifluoroacetic acid salt of 2,2′,2″-(10-(2-((6-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenoxy)piperidin-1-yl)hexyl)amino)-2-oxoethyl)-1,4,7,10-tetraazacyclododecane-1,4,7-triyl)triacetic acid (29.6 mg, 23.8 μmol, 39%) as a white solid. MS: Calc'd for C₅₉H₈₅N₉O₁₃: 1127.63, found [M+H]⁺: 1128.6.

### Example 54: Compound 54

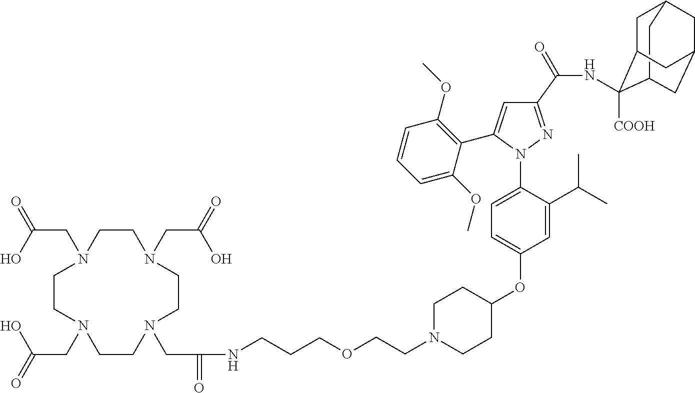 [SMILES: COc1cccc(OC)c1-c1cc(C(=O)NC2(C(=O)O)C3CC4CC(C3)CC2C4)nn1-c1ccc(OC2CCN(CCOCCCNC(=O)CN3CCN(CC(=O)O)CCN(CC(=O)O)CCN(CC(=O)O)CC3)CC2)cc1C(C)C]

Step 1: Into a 40-mL vial, was placed a mixture of tert-butyl (3-(2-hydroxyethoxy)propyl)carbamate (100 mg, 1 Eq, 456 μmol), TEA (231 mg, 318 μL, 5.01 Eq, 2.28 mmol) and DCM (2 mL). The reaction mixture was stirred at 0° C. for 15 minutes, then Ms-Cl (104 mg, 70.7 μL, 1.99 Eq, 908 μmol) was added and the reaction mixture was stirred at 25° C. for an additional 1 hour. The mixture was diluted with water (20 mL), extracted with EtOAc (20 mL×3), the combined organic layers were washed with water (20 mL×2), brine (10 mL), dried over anhydrous Na₂SO₄and concentrated under reduced pressure. The crude product was purified by MPLC to provide 2-(3-((tert-butoxycarbonyl)amino)propoxy)ethyl methanesulfonate (100 mg, 336 mol, 73.7%) as a white solid.

Steps 2-4: Compound 54 was synthesized from tert-butyl (1R,3S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (70 mg, 1 Eq, 0.10 mmol) and 2-(3-((tert-butoxycarbonyl)amino)propoxy)ethyl methanesulfonate (60 μg, 2.0 Eq, 0.20 mmol) in a manner similar to Example 50, Steps 3-5 to provide the trifluoroacetic acid salt of 2,2,2′-(10-(2-((3-(2-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenoxy)piperidin-1-yl)ethoxy)propyl)amino)-2-oxoethyl)-1,4,7,10-tetraazacyclododecane-1,4,7-triyl)triacetic acid (44.7 mg, 35.9 μmol, 46%) as a white solid. MS: Calc'd for C₅₈H₈₃N₉O₁₄: 1129.61, found [M+H]⁺: 1130.6.

### Example 55: Compound 55

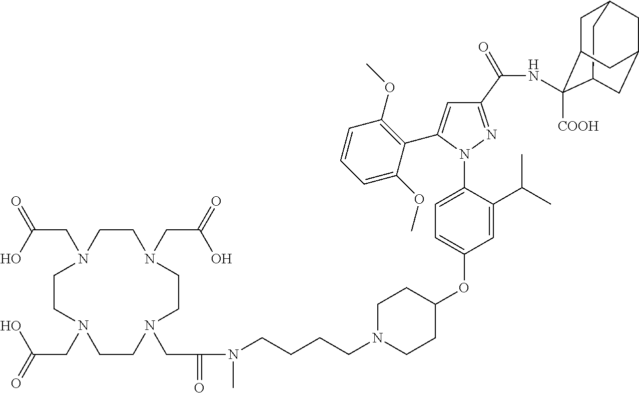 [SMILES: COc1cccc(OC)c1-c1cc(C(=O)NC2(C(=O)O)C3CC4CC(C3)CC2C4)nn1-c1ccc(OC2CCN(CCCCN(C)C(=O)CN3CCN(CC(=O)O)CCN(CC(=O)O)CCN(CC(=O)O)CC3)CC2)cc1C(C)C]

Compound 55 was synthesized from tert-butyl (1R,3S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (60 mg, 1 Eq, 86 μmol) and tert-butyl methyl(4-oxobutyl)carbamate (17 mg, 0.98 Eq, 84 μmol) in a manner similar to Example 19, Step 1 then Example 13, Steps 4 and 5 to provide the trifluoroacetic acid salt of 2,2′,2″-(10-(2-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenoxy)piperidin-1-yl)butyl)(methyl)amino)-2-oxoethyl)-1,4,7,10-tetraazacyclododecane-1,4,7-triyl)triacetic acid (23.1 mg, 18.8 mol, 34%) as a white solid. MS: Calc'd for C₅₈H₈₃N₉O₁₃: 1113.61, found [M+H]⁺: 1114.6.

### Synthesis of Compound 55-Lu

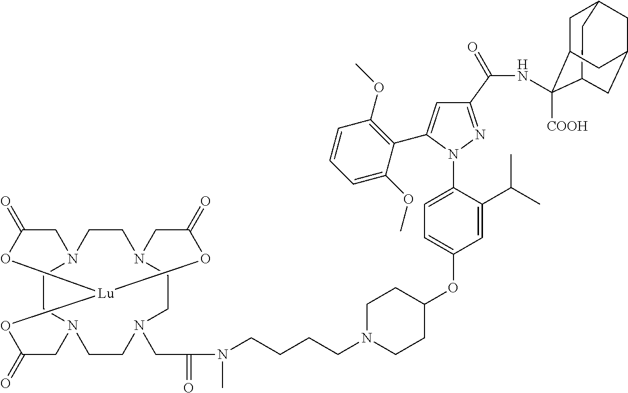

A mixture of 2,2′,2″-(10-(2-((4-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenoxy)piperidin-1-yl)butyl)(methyl)amino)-2-oxoethyl)-1,4,7,10-tetraazacyclododecane-1,4,7-triyl)triacetic acid (30 mg, 1 Eq, 27 μmol), Lutetium (III) chloride (30 mg, 7.5 μL, 4.0 Eq, 0.11 μmol) and NaOAc Buffer (1.5 mL) was stirred at 25° C. for 1 hour. The crude mixture was directly purified by Prep-HPLC to provide the product (16.3 mg, 11.6 μmol, 43%) as white solid. MS: Calc'd for C₅₈H₈₀LuN₉O₁₃: 1285.53, found [M+H]⁺: 1286.5.

### Example 56: Compound 56

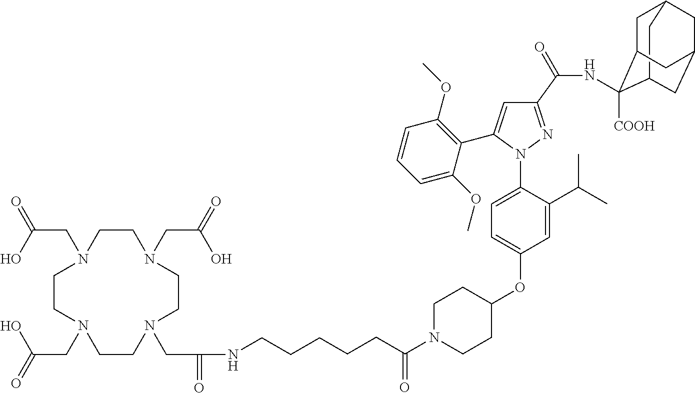 [SMILES: COc1cccc(OC)c1-c1cc(C(=O)NC2(C(=O)O)C3CC4CC(C3)CC2C4)nn1-c1ccc(OC2CCN(C(=O)CCCCCNC(=O)CN3CCN(CC(=O)O)CCN(CC(=O)O)CCN(CC(=O)O)CC3)CC2)cc1C(C)C]

Compound 56 was synthesized from 6-((tert-butoxycarbonyl)amino)hexanoic acid (40 mg, 2.0 Eq, 0.17 mmol) and tert-butyl (1R,3 S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (60 mg, 1 Eq, 86 μmol) in a manner similar to Example 14, Step 2 then Example 13, Steps 4 and 5 to provide the trifluoroacetic acid salt of 2,2′,2″-(10-(2-((6-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenoxy)piperidin-1-yl)-6-oxohexyl)amino)-2-oxoethyl)-1,4,7,10-tetraazacyclododecane-1,4,7-triyl)triacetic acid (53.7 mg, 42.7 μmol, 54%) as a white solid. MS: Calc'd for C₅₉H₈₄N₉O₁₄: 1142.61, found [M+H]⁺: 1143.6.

### Example 57: Compound 57

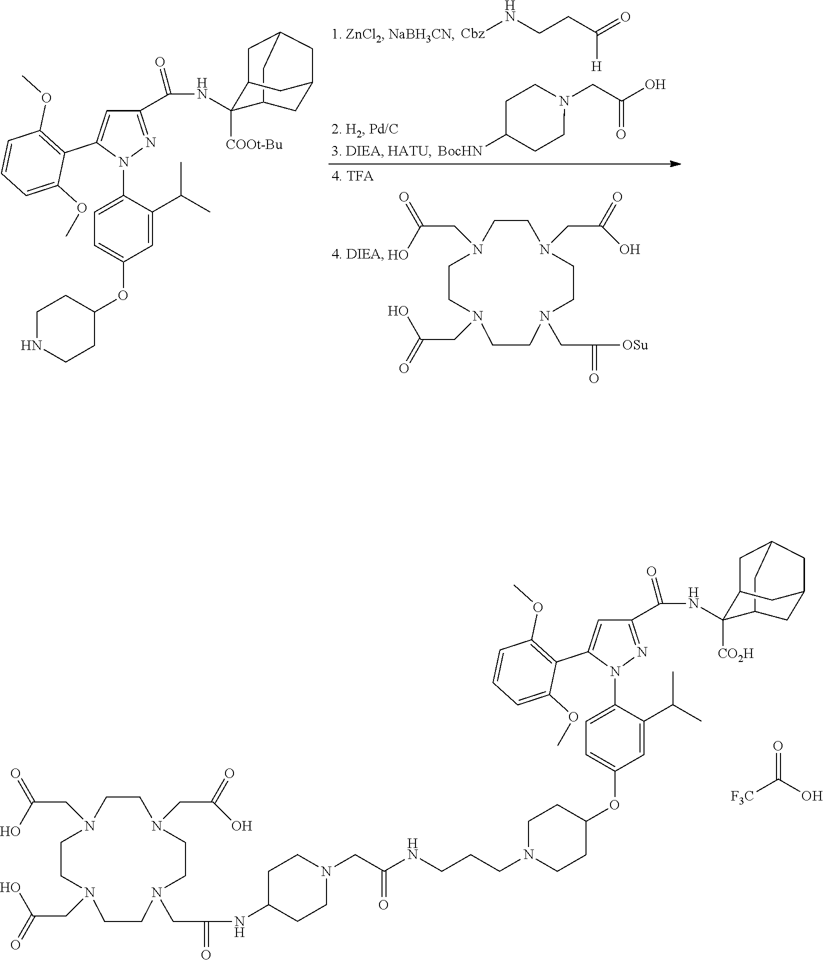

Step 1: Into an 8-mL vial, was placed a mixture of tert-butyl (1R,3S,5r,7r)-2-(5-(2,6-dimethoxyphenyl)-1-(2-isopropyl-4-(piperidin-4-yloxy)phenyl)-1H-pyrazole-3-carboxamido)adamantane-2-carboxylate (100 mg, 1 Eq, 143 mol), benzyl (3-oxopropyl)carbamate (59 mg, 2.0 Eq, 0.28 mmol), zinc chloride (20 mg, 9.3 μL, 1.0 Eq, 0.15 mmol) and MeOH (2 mL). The reaction mixture was stirred at 25° C. for 30 minutes, then sodium cyanoborohydride (18 μg, 17 μL, 2.0 Eq, 0.29 mmol) was added into the reaction mixture and stirred at 25° C. for 1 hour. The crude product was purified by Prep-HPLC to provide tert-butyl (1R,3S,5r,7r)-2-(1-(4-((1-(3-(((benzyloxy)carbonyl)amino)propyl)piperidin-4-yl)oxy)-2-isopropylphenyl)-5-(2,6-dimethoxyphenyl)-1H-pyrazole-3-carboxamido)adamantane-2-carboxylate (100 mg, 112 μmol, 78.5%) as a white solid. [M+H]⁺=890.6.

Step 2: The product from Step 1 was treated with hydrogen and Pd/C in a manner similar to Example 1, Step 7 to provide tert-butyl (1R,3S,5r,7r)-2-(1-(4-((1-(3-aminopropyl)piperidin-4-yl)oxy)-2-isopropylphenyl)-5-(2,6-dimethoxyphenyl)-1H-pyrazole-3-carboxamido)adamantane-2-carboxylate (80 mg, 0.11 mmol, 94%) as a white solid. [M+H]⁺=756.6.

Steps 3-5: Compound 57 was prepared from tert-butyl (1R,3S,5r,7r)-2-(1-(4-((1-(3-aminopropyl)piperidin-4-yl)oxy)-2-isopropylphenyl)-5-(2,6-dimethoxyphenyl)-1H-pyrazole-3-carboxamido)adamantane-2-carboxylate and 2-(4-((tert-butoxycarbonyl)amino)piperidin-1-yl)acetic acid in a manner similar to Example 14, Step 2, then Example 13, Steps 4 and 5 to provide the 2,2,2-trifluoroacetic acid salt of 2,2′,2″-(10-(2-((1-(2-((3-(4-(4-(5-(2,6-dimethoxyphenyl)-3-(((1R,3 S,5r,7r)-2-(hydroxy(oxo)-15-methyl)adamantan-2-yl)carbamoyl)-1H-pyrazol-1-yl)-3-isopropylphenoxy)piperidin-1-yl)propyl)amino)-2-oxoethyl)piperidin-4-yl)amino)-2-oxoethyl)-1,4,7,10-tetraazacyclododecane-1,4,7-triyl)triacetic acid (50.2 mg, 37.4 μmol, 63%) as a white solid. MS: Calc'd for C₆₃H₉N₁₁O₁₄: 1225.67, found [M+H]⁺: 1226.7.

### Synthesis of Compound 57-Lu

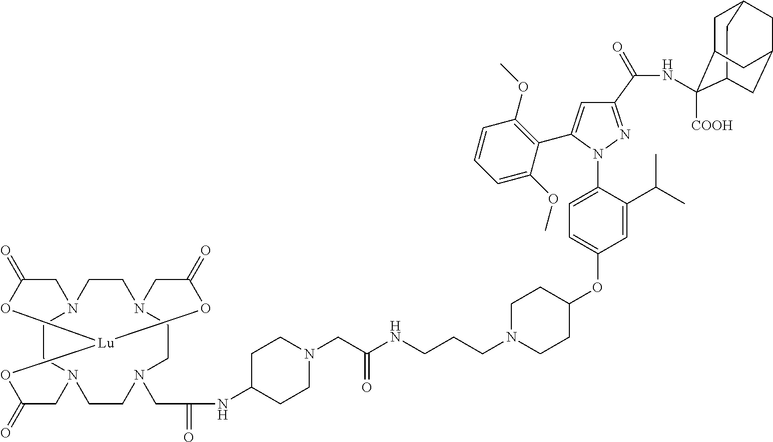

A mixture of 2,2′,2″-(10-(2-((1-(2-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenoxy)piperidin-1-yl)propyl)amino)-2-oxoethyl)piperidin-4-yl)amino)-2-oxoethyl)-1,4,7,10-tetraazacyclododecane-1,4,7-triyl)triacetic acid (30 mg, 1 Eq, 24 μmol), Lutetium (III) chloride (28 mg, 7.0 μL, 4.1 Eq, 0.10 mmol) and NaOAc/AcOH Buffer (1.5 mL) was stirred at 70° C. for 30 min. The crude mixture was directly purified by Prep-HPLC to provide the product (22.3 mg, 14.7 μmol, 60%) as a white solid. MS: Calc'd for C₆₃H₈₈LuN₁₁O₁₄: 1397.59, found [M+H]⁺: 1398.6.

### Example 58: Compound 58

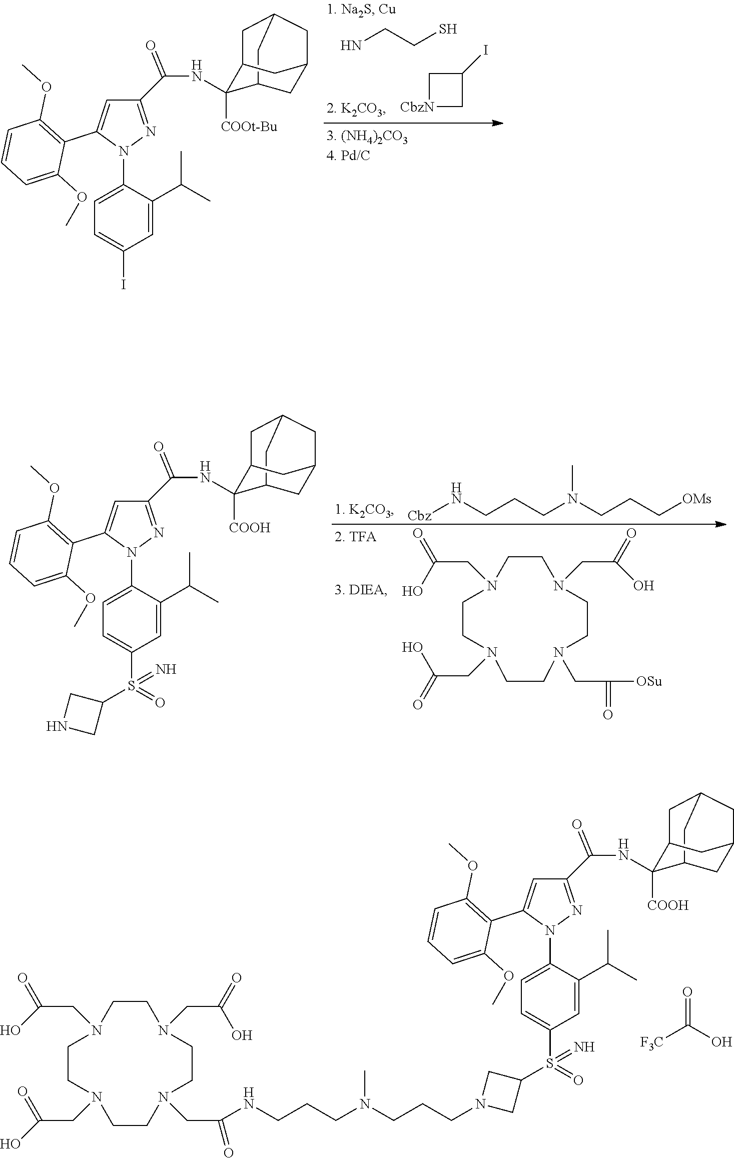

Step 1: Into a 40-mL vial, was placed a mixture of tert-butyl (1R,3 S,5r,7r)-2-(5-(2,6-dimethoxyphenyl)-1-(4-iodo-2-isopropylphenyl)-1H-pyrazole-3-carboxamido)adamantane-2-carboxylate (850 mg, 1 Eq, 1.17 mmol), ethane-1,2-dithiol (0.3 g, 3 Eq, 3 mmol), Na₂S (0.2 g, 2 Eq, 3 mmol) and DMSO (13 mL). The reaction mixture was stirred at 25° C. for 1 hour. The mixture was directly purified by MPLC to provide tert-butyl (1R,3S,5r,7r)-2-(5-(2,6-dimethoxyphenyl)-1-(2-isopropyl-4-mercaptophenyl)-1H-pyrazole-3-carboxamido)adamantane-2-carboxylate (355 mg, 562 μmol, 48.0%) as a yellow oil. [M+H]⁺632.4.

Step 2: Into a 40-mL vial, was placed a mixture of tert-butyl (1R,3S,5r,7r)-2-(5-(2,6-dimethoxyphenyl)-1-(2-isopropyl-4-mercaptophenyl)-1H-pyrazole-3-carboxamido)adamantane-2-carboxylate (355 mg, 1 Eq, 562 μmol), K₂CO₃ (262 mg, 3.37 Eq, 1.90 mmol), benzyl 3-iodoazetidine-1-carboxylate (301 mg, 1.69 Eq, 949 μmol) and DMF (6 mL). The reaction mixture was stirred at 80° C. for 1 hour. The mixture was directly purified by MPLC to afford benzyl 3-((4-(3-(((1R,3 S,5r,7r)-2-(tert-butoxycarbonyl)adamantan-2-yl)carbamoyl)-5-(2,6-dimethoxyphenyl)-1H-pyrazol-1-yl)-3-isopropylphenyl)thio)azetidine-1-carboxylate (320 mg, 390 μmol, 69.4%) as a yellow oil. [M+H]⁺=821.5.

Step 3: Into a 40-mL vial, was placed a mixture of benzyl 3-((4-(3-(((1R,3S,5r,7r)-2-(tert-butoxycarbonyl)adamantan-2-yl)carbamoyl)-5-(2,6-dimethoxyphenyl)-11H-pyrazol-1-yl)-3-isopropylphenyl)thio)azetidine-1-carboxylate (500 mg, 1 Eq, 609 μmol), (NH₄)₂CO₃ (250 mg, 3.00 Eq, 1.82 mmol) and EtOH (8 mL). The reaction mixture was stirred at 25° C. for 1 hour. The mixture was directly purified by MPLC to provide benzyl 3-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenylsulfonimidoyl)azetidine-1-carboxylate (380 mg, 446 μmol, 73.2%) as a yellow oil. [M+H]⁺=852.5.

Step 4: The product from Step 3 was treated with hydrogen and Pd/C in a manner similar to Example 1, Step 7 to provide tert-butyl (1R,3S,5r,7r)-2-(1-(4-(azetidine-3-sulfonimidoyl)-2-isopropylphenyl)-5-(2,6-dimethoxyphenyl)-1H-pyrazole-3-carboxamido)adamantane-2-carboxylate (290 mg, 0.30 mmol, 81%) as a white solid. [M+H]⁺=718.4.

Steps 5-7: Compound 58 was synthesized from tert-butyl (1R,3S,5r,7r)-2-(1-(4-(azetidine-3-sulfonimidoyl)-2-isopropylphenyl)-5-(2,6-dimethoxyphenyl)-1H-pyrazole-3-carboxamido)adamantane-2-carboxylate and 3-((3-(((benzyloxy)carbonyl)amino)propyl)(methyl)amino)propyl methanesulfonate in a manner similar to Example 50, Steps 3-5 to afford 2,2′,2″-(10-(2-((3-((3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenylsulfonimidoyl)azetidin-1-yl)propyl)(methyl)amino)propyl)amino)-2-oxoethyl)-1,4,7,10-tetraazacyclododecane-1,4,7-triyl)triacetic acid (5.6 mg, 4.8 μmol, 29%) as a white solid. MS: Calc'd for C₅₈H₈₅N₁₁O₁₃S: 1175.60, found [M+1H]: 1176.6.

### Example 59: Compound 59

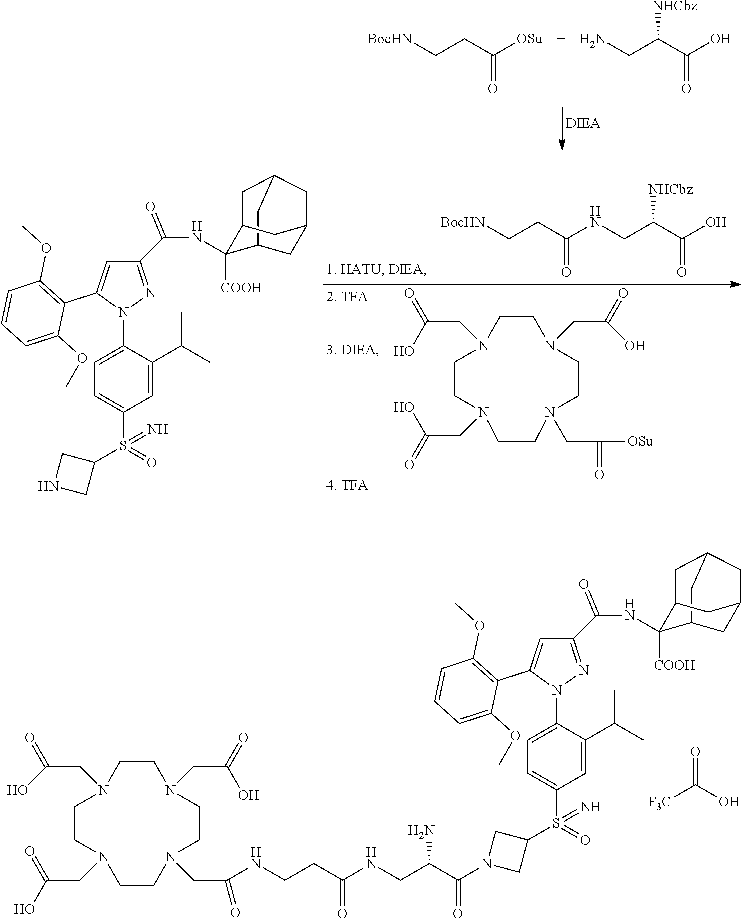

Step 1: Into an 8-mL vial, was placed a mixture of 2,5-dioxopyrrolidin-1-yl 3-((tert-butoxycarbonyl)amino)propanoate (200 mg, 1 Eq, 699 μmol), (S)-3-amino-2-(((benzyloxy)carbonyl)amino)propanoic acid (250 mg, 1.50 Eq, 1.05 mmol) and DMF (1.5 mL), to which was added DIEA (271 mg, 365 μL, 3.00 Eq, 2.10 mmol). The reaction mixture was stirred at 25° C. for 1 hour. The mixture was directly purified by MPLC to provide (S)-2-(((benzyloxy)carbonyl)amino)-3-(3-((tert-butoxycarbonyl)amino)propanamido)propanoic acid (300 mg, 733 mol, 105%) as a white solid. [M+H]⁺=410.1.

Steps 2-4: The product was synthesized from (S)-2-(((benzyloxy)carbonyl)amino)-3-(3-((tert-butoxycarbonyl)amino)propanamido)propanoic acid and tert-butyl (1R,3S,5r,7r)-2-(1-(4-(azetidine-3-sulfonimidoyl)-2-isopropylphenyl)-5-(2,6-dimethoxyphenyl)-1H-pyrazole-3-carboxamido)adamantane-2-carboxylate in a manner similar to Example 14, Step 2 then Example 13, Steps 4 and 5 to provide the trifluoroacetic acid salt of 2,2′,2″-(10-((5S)-5-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenylsulfonimidoyl)azetidine-1-carbonyl)-3,8,12-trioxo-1-phenyl-2-oxa-4,7,11-triazatridecan-13-yl)-1,4,7,10-tetraazacyclododecane-1,4,7-triyl)triacetic acid (2 mg, 1 mol, 9%) as a white solid. [M+H-TFA]=1339.8.

Step 5: The product from Step 4 was treated with TFA in a manner similar to Example 13, Steps 4 to provide the 2,2,2-trifluoroacetic acid salt of 2,2′,2″-(10-(2-((3-(((2S)-2-amino-3-(3-(4-(3-(((1R,3 S,5r,7r)-2-carboxyadamantan-2-yl)carbamoyl)-5-(2,6-dimethoxyphenyl)-1H-pyrazol-1-yl)-3-isopropylphenylsulfonimidoyl)azetidin-1-yl)-3-oxopropyl)amino)-3-oxopropyl)amino)-2-oxoethyl)-1,4,7,10-tetraazacyclododecane-1,4,7-triyl)triacetic acid (1.9 mg, 1.4 μmol, 100%) as a white solid. MS: Calc'd for C₅₇H₈N₁₂O₁₅S: 1204.56, found [M+H]⁺: 1205.6.

### Example 60: Compound 60

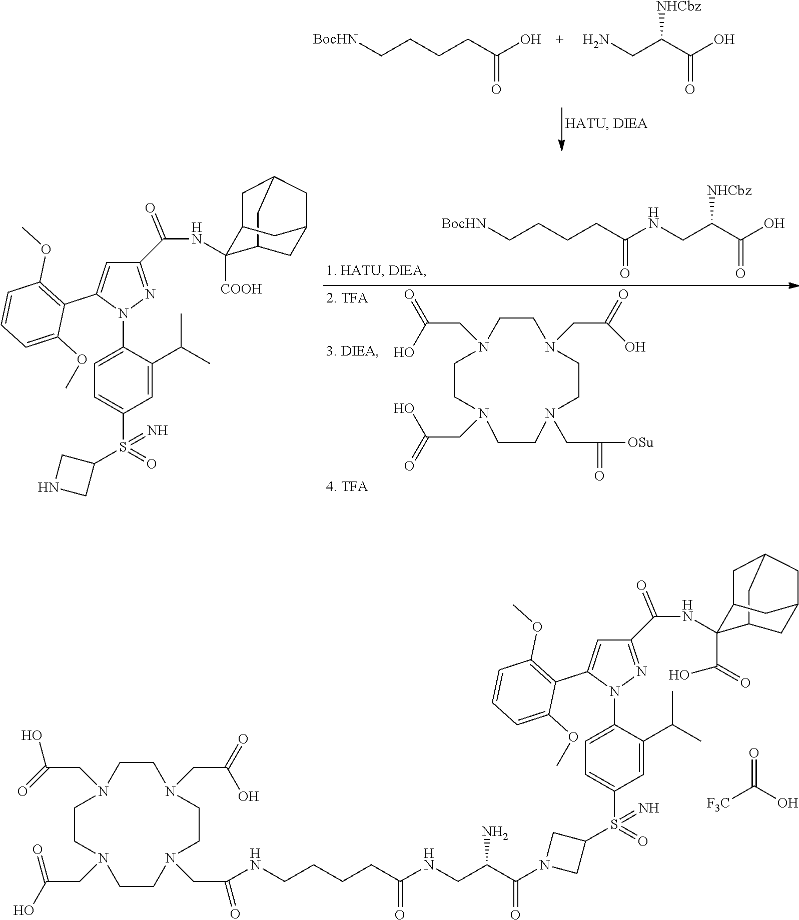

Step 1: Into a 40-mL vial, was placed a mixture of 5-((tert-butoxycarbonyl)amino)pentanoic acid (200 mg, 1 Eq, 921 μmol), HATU (420 mg, 1.20 Eq, 1.10 mmol), DIEA (357 mg, 481 μL, 3.00 Eq, 2.76 mmol) and DMF (1.5 mL). The reaction mixture was stirred at 20° C. for 10 minutes, then (S)-3-amino-2-(((benzyloxy)carbonyl)amino)propanoic acid (219 mg, 0.999 Eq, 919 μmol) was added and the reaction mixture was stirred at 25° C. for 1 hour. The mixture was directly purified by MPLC to provide (S)-2-(((benzyloxy)carbonyl)amino)-3-(5-((tert-butoxycarbonyl)amino)pentanamido)propanoic acid as a yellow oil. [M+H]=438.2.

Steps 2-5: Compound 60 was synthesized from (S)-2-(((benzyloxy)carbonyl)amino)-3-(5-((tert-butoxycarbonyl)amino)pentanamido)propanoic acid and tert-butyl (1R,3S,5r,7r)-2-(1-(4-(azetidine-3-sulfonimidoyl)-2-isopropylphenyl)-5-(2,6-dimethoxyphenyl)-1H-pyrazole-3-carboxamido)adamantane-2-carboxylate in a manner similar to Example 14, Step 2 then Example 13, Steps 4 and 5, The Example 13, Step 4 to provide the trifluoroacetic acid salt of 2,2′,2″-(10-(2-((5-(((2S)-2-amino-3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenylsulfonimidoyl)azetidin-1-yl)-3-oxopropyl)amino)-5-oxopentyl)amino)-2-oxoethyl)-1,4,7,10-tetraazacyclododecane-1,4,7-triyl)triacetic acid (5.6 mg, 4.2 μmol, 28%) as a white solid. MS: Calc'd for C₅₉H₈₄N₁₂O₁₅S: 1232.59, found [M+H]⁺: 1233.6.

### Example 61: Compound 61

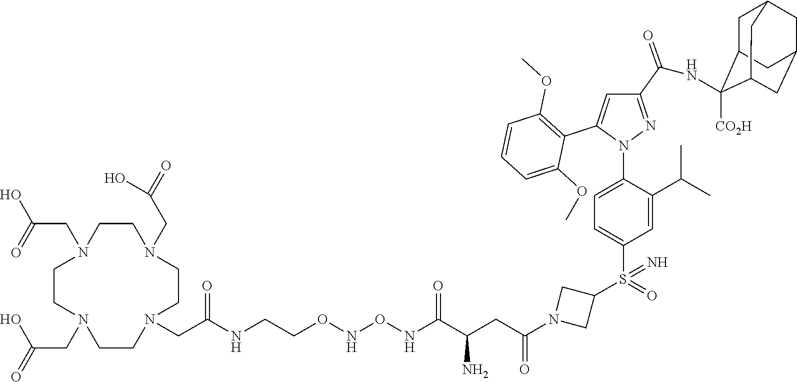 [SMILES: COc1cccc(OC)c1-c1cc(C(=O)NC2(C(=O)O)C3CC4CC(C3)CC2C4)nn1-c1ccc(S(=N)(=O)C2CN(C(=O)C[C@@H](N)C(=O)NONOCCNC(=O)CN3CCN(CC(=O)O)CCN(CC(=O)O)CCN(CC(=O)O)CC3)C2)cc1C(C)C]

### Example 62: Compound 62

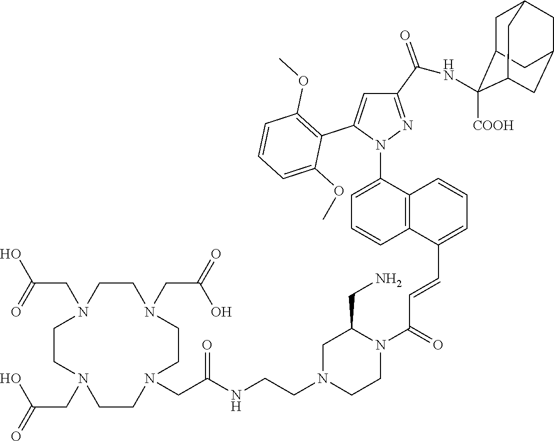 [SMILES: COc1cccc(OC)c1-c1cc(C(=O)NC2(C(=O)O)C3CC4CC(C3)CC2C4)nn1-c1cccc2c(/C=C/C(=O)N3CCN(CCNC(=O)CN4CCN(CC(=O)O)CCN(CC(=O)O)CCN(CC(=O)O)CC4)C[C@H]3CN)cccc12]

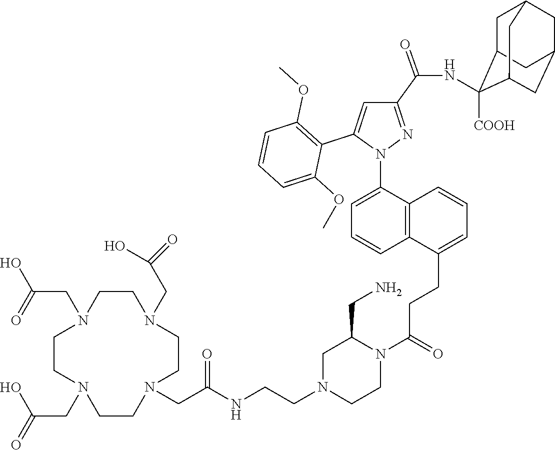 [SMILES: COc1cccc(OC)c1-c1cc(C(=O)NC2(C(=O)O)C3CC4CC(C3)CC2C4)nn1-c1cccc2c(CCC(=O)N3CCN(CCNC(=O)CN4CCN(CC(=O)O)CCN(CC(=O)O)CCN(CC(=O)O)CC4)C[C@H]3CN)cccc12]

### Example 63: Compound 63

### Example 64: Compound 64

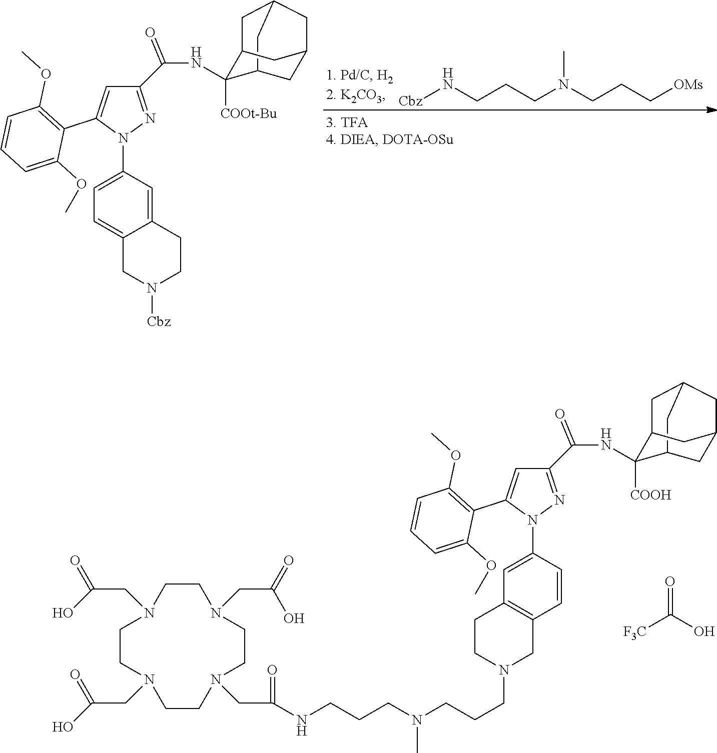

Step 1: Into a 50-mL round-bottom flask under an inert atmosphere of nitrogen, was placed benzyl 6-(3-(((1R,3 S,5r,7r)-2-(tert-butoxycarbonyl)adamantan-2-yl)carbamoyl)-5-(2,6-dimethoxyphenyl)-1H-pyrazol-1-yl)-3,4-dihydroisoquinoline-2(1H)-carboxylate (80 mg, 1 Eq, 0.11 mmol) and MeOH (2 mL), to which was carefully added Pd/C (50 mg, 4.4 Eq, 0.47 mmol). The flask was evacuated and flushed with hydrogen three times. The mixture was stirred for 1 hour at 25° C. The reaction mixture was filtered through a pad of celite, the filtrate was concentrated and purified by MPLC to provide tert-butyl (1R,3S,5r,7r)-2-(5-(2,6-dimethoxyphenyl)-1-(1,2,3,4-tetrahydroisoquinolin-6-yl)-1H-pyrazole-3-carboxamido)adamantane-2-carboxylate (60 mg, 98 nmol, 91%) as a white solid.

Step 2: Compound 64 was synthesized from tert-butyl (1R,3S,5r,7r)-2-(5-(2,6-dimethoxyphenyl)-1-(1,2,3,4-tetrahydroisoquinolin-6-yl)-1H-pyrazole-3-carboxamido)adamantane-2-carboxylate and benzyl (3-((3-hydroxypropyl)(methyl)amino)propyl)carbamate in a manner similar to Example 13, Step 3-5 to provide the 2,2,2-trifluoroacetic acid salt of 2,2′,2″-(10-(2-((3-((3-(6-(3-(((1R,3S,5r,7r)-2-carboxyadamantan-2-yl)carbamoyl)-5-(2,6-dimethoxyphenyl)-1H-pyrazol-1-yl)-3,4-dihydroisoquinolin-2(1H)-yl)propyl)(methyl)amino)propyl)amino)-2-oxoethyl)-1,4,7,10-tetraazacyclododecane-1,4,7-triyl)triacetic acid (36 mg, 30 μmol, 55%) as a white solid. MS: Calc'd for C₅₅H₇N₁₀O₁₂: 1070.58, found [M+H]⁺: 1071.6.

### Example 65: Compound 65

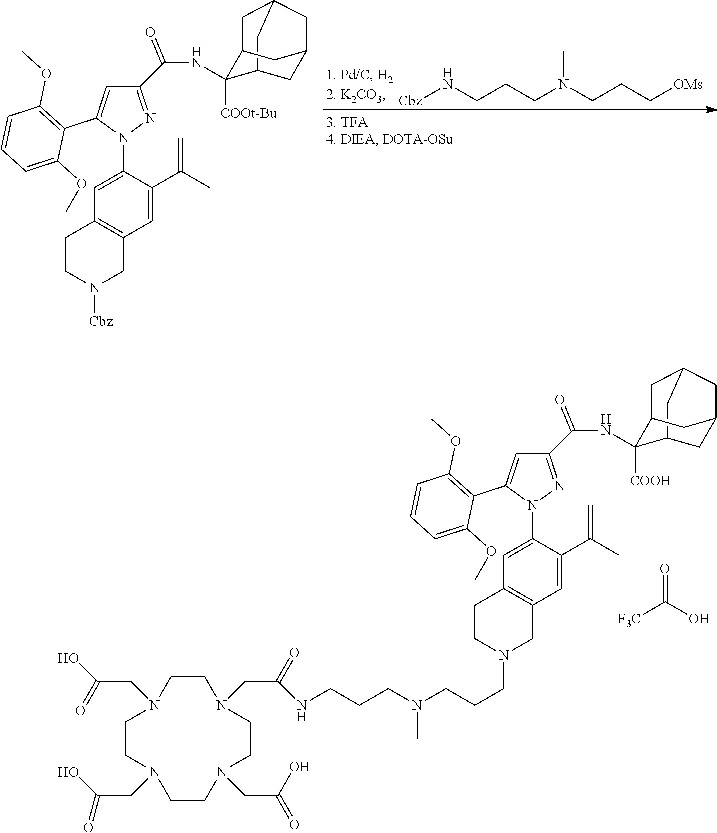

Step 1: Into a 50-mL round-bottom flask under an inert atmosphere of nitrogen, was placed benzyl 6-(3-(((11R,3 S,5r,7r)-2-(tert-butoxycarbonyl)adamantan-2-yl)carbamoyl)-5-(2,6-dimethoxyphenyl)-1H-pyrazol-1-yl)-7-(prop-1-en-2-yl)-3,4-dihydroisoquinoline-2(1H)-carboxylate (100 mg, 1 Eq, 127 μmol) and MeOH (3 mL) to which was carefully added Pd/C (8.11 mg, 0.600 Eq, 76.2 μmol). The flask was evacuated and flushed with hydrogen three times. The mixture was stirred for 25° C. at 20 min. The reaction mixture was filtered through a pad of celite, then the filtrate was concentrated and purified by MPLC to afford tert-butyl (1R,3 S,5r,7r)-2-(5-(2,6-dimethoxyphenyl)-1-(7-(prop-1-en-2-yl)-1,2,3,4-tetrahydroisoquinolin-6-yl)-1H-pyrazole-3-carboxamido)adamantane-2-carboxylate (50 mg, 77 mol, 60%) as a white solid. [M+H]⁺: 653.7.

Steps 2-4: Compound 64 was synthesized from tert-butyl (1R,3S,5r,7r)-2-(5-(2,6-dimethoxyphenyl)-1-(7-(prop-1-en-2-yl)-1,2,3,4-tetrahydroisoquinolin-6-yl)-1H-pyrazole-3-carboxamido)adamantane-2-carboxylate and 3-((3-(((benzyloxy)carbonyl)amino)propyl)(methyl)amino)propyl methanesulfonate in a manner similar to Example 13, Steps 3-5 to provide the 2,2,2-trifluoroacetic acid salt of 2,2′,2″-(10-(2-((3-((3-(6-(3-(((1R,3S,5r,7r)-2-carboxyadamantan-2-yl)carbamoyl)-5-(2,6-dimethoxyphenyl)-1H-pyrazol-1-yl)-7-(prop-1-en-2-yl)-3,4-dihydroisoquinolin-2(1H)-yl)propyl)(methyl)amino)propyl)amino)-2-oxoethyl)-1,4,7,10-tetraazacyclododecane-1,4,7-triyl)triacetic acid (14.3 mg, 11.7 μmol, 28%) as a white solid. MS: Calc'd for C₈₈H₈₂N₁₀O₁₂: 1110.61, found [M+H]⁺: 1111.6.

### Example 66: Compound 66

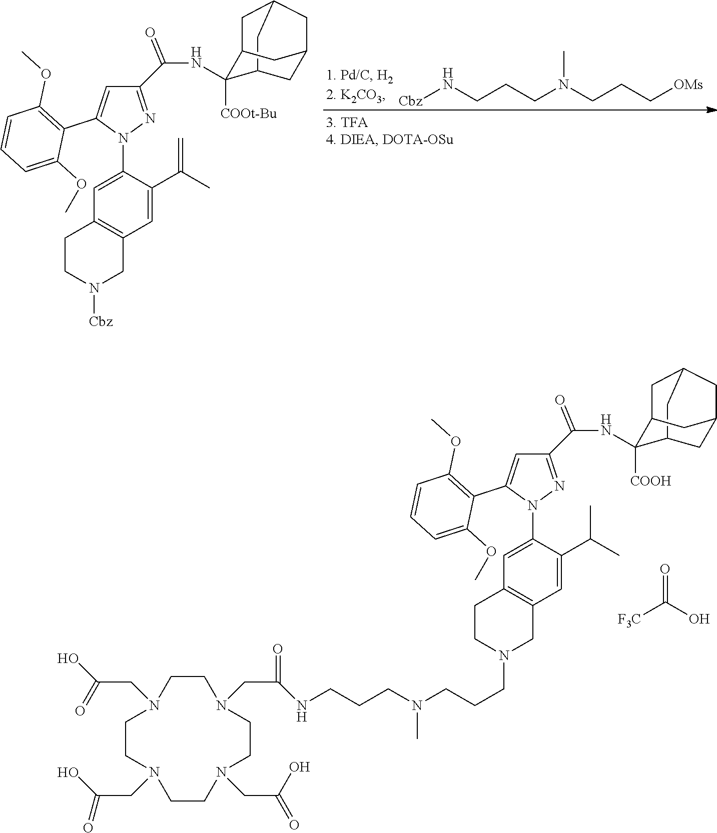

Step 1: Into a 50-mL round-bottom flask under an inert atmosphere of nitrogen, was placed benzyl 6-(3-(((1R,3 S,5r,7r)-2-(tert-butoxycarbonyl)adamantan-2-yl)carbamoyl)-5-(2,6-dimethoxyphenyl)-1H-pyrazol-1-yl)-7-(prop-1-en-2-yl)-3,4-dihydroisoquinoline-2(1H)-carboxylate (110 mg, 1 Eq, 140 μmol) and MeOH (3 mL) to which was carefully added Pd/C (8.93 mg, 0.600 Eq, 83.9 μmol). The flask was evacuated and flushed with hydrogen three times. The mixture was stirred for 25° C. at 3 hours. The reaction mixture was filtered through a pad of celite, the filtrate was concentrated and purified by MPLC to provide tert-butyl (1R,3 S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate (70 mg, 86 mol, 61%) as a white solid. [M+H]⁺=655.5.

Steps 2-4: Compound 66 was synthesized from tert-butyl (1R,3S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate and benzyl (3-((3-hydroxypropyl)(methyl)amino)propyl)carbamate in a manner similar to Example 13, Steps 3-5 to provide the 2,2,2-trifluoroacetic acid salt of 2,2′,2″-(10-(2-((3-((3-(6-(3-(((1R,3S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(1H)-yl)propyl)(methyl)amino)propyl)amino)-2-oxoethyl)-1,4,7,10-tetraazacyclododecane-1,4,7-triyl)triacetic acid (4.1 mg, 3.3 μmol, 16%) as a white solid. MS: Calc'd for C₅₈H₈₄N₁₀O₁₂: 1112.63, found [M+H]⁺: 1113.6.

### Example 67: Compound 67

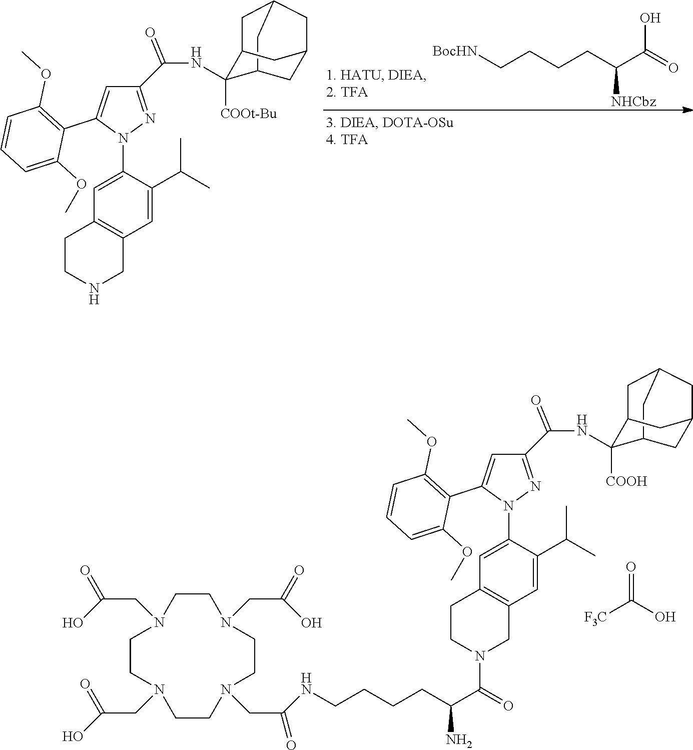

Step 1: A mixture of N2-((benzyloxy)carbonyl)-N6-(tert-butoxycarbonyl)-L-lysine (20 mg, 0.98 Eq, 53 μmol), HATU (20 mg, 0.98 Eq, 53 μmol), DIEA (21 mg, 28 μL, 3.0 Eq, 0.16 mmol) and DMF (1 mL) was stirred at 20° C. for 10 minutes, then tert-butyl (1R,3S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate (35 mg, 1 Eq, 53 μmol) was added and the reaction mixture was stirred at 25° C. for an additional 1 hour. The mixture was directly purified by MPLC to afford tert-butyl (1R,3S,5r,7r)-2-(1-(2-(N2-((benzyloxy)carbonyl)-N6-(tert-butoxycarbonyl)-L-lysyl)-7-isopropyl-1,2,3,4-tetrahydroisoquinolin-6-yl)-5-(2,6-dimethoxyphenyl)-1H-pyrazole-3-carboxamido)adamantane-2-carboxylate (30 mg, 29 μmol, 55%) as a yellow oil. [M+H]⁺:=1017.9.

Steps 2-4: tert-Butyl (1R,3S,5r,7r)-2-(1-(2-(N2-((benzyloxy)carbonyl)-N6-(tert-butoxycarbonyl)-L-lysyl)-7-isopropyl-1,2,3,4-tetrahydroisoquinolin-6-yl)-5-(2,6-dimethoxyphenyl)-1H-pyrazole-3-carboxamido)adamantane-2-carboxylate (30 mg, 1 Eq, 29 μmol) was treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5, then treated with TFA in a manner similar Example 13, Step 4 to afford 2,2′,2″-(10-(2-(((S)-5-amino-6-(6-(3-(((1R,3S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(1H)-yl)-6-oxohexyl)amino)-2-oxoethyl)-1,4,7,10-tetraazacyclododecane-1,4,7-triyl)triacetic acid (16.7 mg, 13.6 μmol, 85%) as a white solid. MS: Calc'd for C₅₇H₈₀N₁₀O₁₃: 1112.59, found [M+H]v: 1113.5.

### Example 68: Compound 68

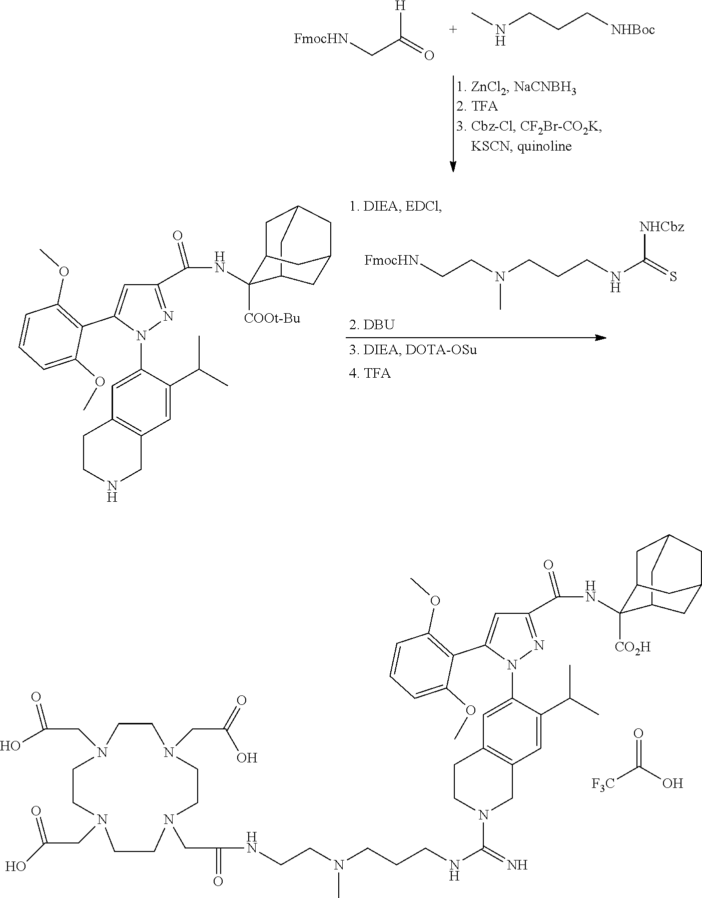

Step 1: (9H-fluoren-9-yl)methyl (2-oxoethyl)carbamate and tert-butyl (3-(methylamino)propyl)carbamate were treated with ZnCl₂ and NaCNBH₃ in a manner similar to Example 19, Step 1 to provide tert-butyl (3-((2-((((9H-fluoren-9-yl)methoxy)carbonyl)amino)ethyl)(methyl)amino)propyl)carbamate (420 mg, 926 μmol, 52.1%) as a yellow oil.

Step 2: The product from Step 2 was treated with TFA in a manner similar to Example 13, Steps 4 to provide (9H-fluoren-9-yl)methyl (2-((3-aminopropyl)(methyl)amino)ethyl)carbamate (280 mg, 792 μmol, 85.5%) as a white solid.

Step 3: Into a 40-mL vial, was placed a mixture of potassium thiocyanate (137 mg, 80.9 μL, 1.99 Eq, 1.41 mmol), potassium 2-bromo-2,2-difluoroacetate (301 mg, 2.00 Eq, 1.41 mmol), quinoline (183 mg, 168 μL, 2.00 Eq, 1.42 mmol) and water (5 mL). Then Cbz-Cl (241 mg, 202 μL, 2.00 Eq, 1.41 mmol) was added and the reaction mixture was stirred at 0° C. for 2 hours. Then (9H-fluoren-9-yl)methyl (2-((3-aminopropyl)(methyl)amino)ethyl)carbamate (250 mg, 1 Eq, 707 μmol) and DCM (5 mL) was added and the reaction mixture was stirred at 25° C. for an additional 2 hours. The mixture was directly purified by MPLC to provide the desired product (45 mg, 82 μmol, 12%) as a yellow oil.

Step 4: tert-Butyl (1R,3S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate (40 mg, 1 Eq, 61 μmol), the product from Step 3 (33 mg, 0.99 Eq, 60 μmol), DIPEA (24 mg, 32 μL, 3.0 Eq, 0.19 mmol), 1-(3-dimethylaminopropyl)-3-ethylcarbodiimide hydrochloride (EDCI) (23 mg, 2.0 Eq, 0.12 mmol) and DCM (2 mL) were combined under an atmosphere of N. The reaction mixture was stirred at 25° C. for 16 hours. The mixture was concentrated under reduced pressure and the crude product was purified by Prep-HPLC to provide tert-butyl (1R,3S,5r,7r)-2-(1-(2-((Z)—N-(3-((2-((((9H-fluoren-9-yl)methoxy)carbonyl)amino)ethyl)(methyl)amino)propyl)-N′-((benzyloxy)carbonyl)carbamimidoyl)-7-isopropyl-1,2,3,4-tetrahydroisoquinolin-6-yl)-5-(2,6-dimethoxyphenyl)-1H-pyrazole-3-carboxamido)adamantane-2-carboxylate (38 mg, 33 μmol, 53%) as a white solid.

Step 5: Into a 8-mL vial, was placed a mixture of tert-butyl (1R,3 S,5r,7r)-2-(1-(2-((Z)—N-(3-((2-((((9H-fluoren-9-yl)methoxy)carbonyl)amino)ethyl)(methyl)amino)propyl)-N′-((benzyloxy)carbonyl)carbamimidoyl)-7-isopropyl-1,2,3,4-tetrahydroisoquinolin-6-yl)-5-(2,6-dimethoxyphenyl)-1H-pyrazole-3-carboxamido)adamantane-2-carboxylate (38 mg, 1 Eq, 33 μmol) and DMF (2 mL), to which was added DBU (40 mg, 40 μL, 8.1 Eq, 0.26 mmol). The reaction mixture was stirred at 25° C. for 1 hour to provide tert-butyl (1R,3 S,5r,7r)-2-(1-(2-((Z)—N-(3-((2-aminoethyl)(methyl)amino)propyl)-N′-((benzyloxy)carbonyl)carbamimidoyl)-7-isopropyl-1,2,3,4-tetrahydroisoquinolin-6-yl)-5-(2,6-dimethoxyphenyl)-1H-pyrazole-3-carboxamido)adamantane-2-carboxylate (25 mug, 26 μmol, 81%), which was used directly in the next step without further purification. Steps 6-7: The product from Step 5 was treated with was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA, then TFA in a manner similar to Example 13, Steps 5 and 4 to provide the 2,2,2-trifluoroacetic acid salt of 2,2′2-(10-(2-((2-((3-(6-(3-(((1R,3S,5r,7r)-2-carboxyadamantan-2-yl)carbamoyl)-5-(2,6-dimethoxyphenyl)-1H-pyrazol-1-yl)-7-isopropyl-1,2,3,4-tetrahydroisoquinoline-2-carboximidamido)propyl)(methyl)amino)ethyl)amino)-2-oxoethyl)-1,4,7,10-tetraazacyclododecane-1,4,7-triyl)triacetic acid (16.8 mg, 13.4 μmol, 74%) as a white solid. MS: Calc'd for C₅₈H₈₄N₁₀O₁₂: 1140.63, found [M+H]⁺: 1141.7.

### Example 69: Compound 69

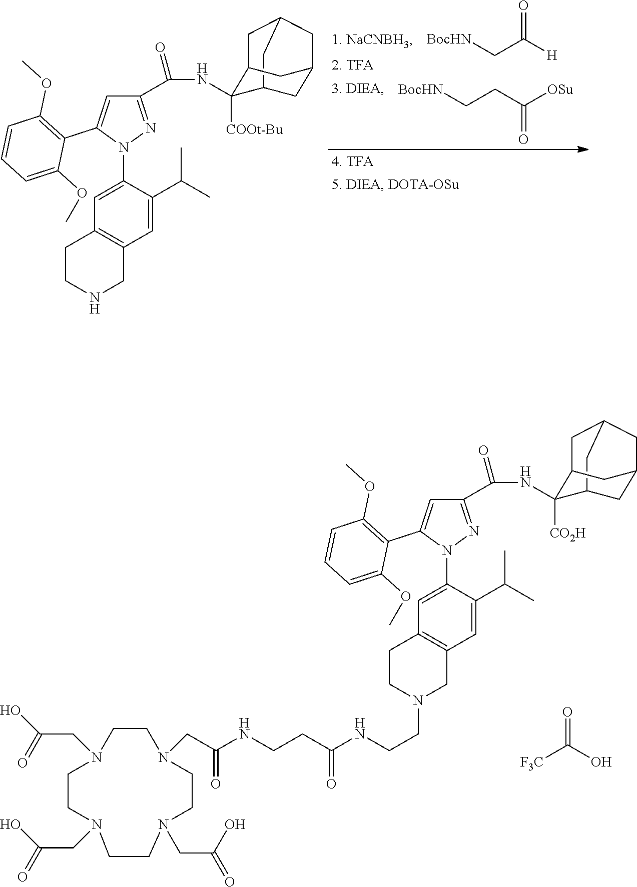

Step 1: Into a 8-mL vial, was placed a mixture of tert-butyl (1R,3S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate (200 mg, 1 Eq, 305 μmol), tert-butyl (2-oxoethyl)carbamate (97 mg, 2.0 Eq, 0.61 mmol), zinc chloride (83 mg, 38 μL, 2.0 Eq, 0.61 mmol) and MeOH (2 mL). The reaction mixture was stirred at 20° C. for 10 minutes, then NaBH₃CN (39 mg, 2.0 Eq, 0.62 mmol) was added and the reaction mixture was stirred at 25° C. for 1 hour. The mixture was directly purified by MPLC to provide tert-butyl (1R,3 S,5r,7r)-2-(1-(2-(2-((tert-butoxycarbonyl)amino)ethyl)-7-isopropyl-1,2,3,4-tetrahydroisoquinolin-6-yl)-5-(2,6-dimethoxyphenyl)-1H-pyrazole-3-carboxamido)adamantane-2-carboxylate (100 mg, 125 μmol, 41.0%) as a yellow oil. [M+H]⁺=798.7.

Step 2: The product from step 1 was treated with TFA in a manner similar to Example 13, Step 4 to provide the 2,2,2-trifluoroacetic acid salt of (1R,3S,5r,7r)-2-(1-(2-(2-aminoethyl)-7-isopropyl-1,2,3,4-tetrahydroisoquinolin-6-yl)-5-(2,6-dimethoxyphenyl)-1H-pyrazole-3-carboxamido)adamantane-2-carboxylic acid (100 mg, 0.12 mmol, 99%) as a yellow oil.

Steps 3-5: The product from Step 2 was treated with 2,5-dioxopyrrolidin-1-yl 3-((tert-butoxycarbonyl)amino)propanoate and DIEA in a manner similar to Example 59, Step 1, then treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((2-(6-(3-(((1R,3S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(1LH)-yl)ethyl)amino)-3-oxopropyl)amino)-2-oxoethyl)-1,4,7,10-tetraazacyclododecane-1,4,7-triyl)triacetic acid (19.7 mg, 16.2 mol, 58%) as a white solid. MS: Calc'd for C₅₆1H₇₈N₁₀O₁₃: 1098.57, found [M+H]⁺: 1099.5.

### Example 70: Compound 70

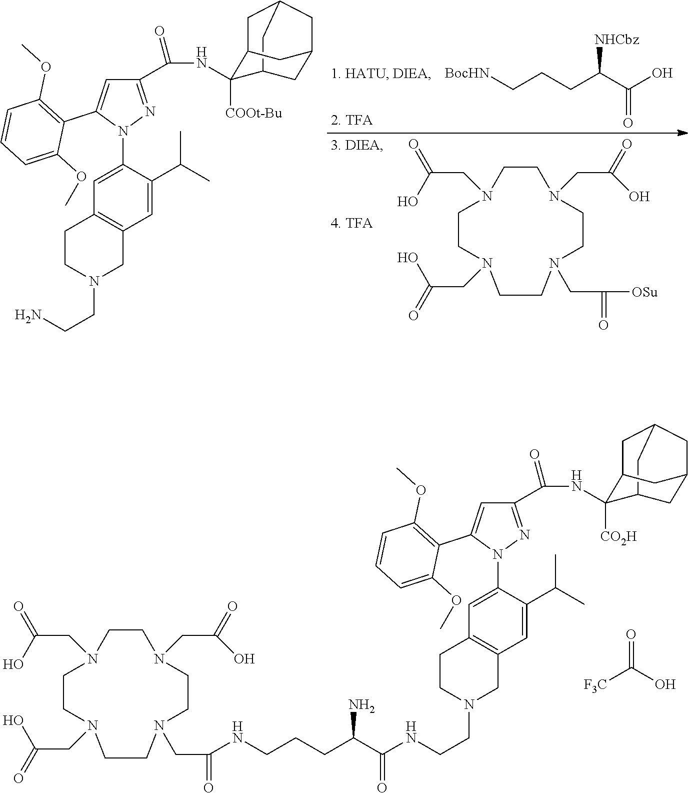

Step 1: (1R,3S,5r,7r)-2-(1-(2-(2-aminoethyl)-7-isopropyl-1,2,3,4-tetrahydroisoquinolin-6-yl)-5-(2,6-dimethoxyphenyl)-1H-pyrazole-3-carboxamido)adamantane-2-carboxylic acid was treated with (R)-2-(((benzyloxy)carbonyl)amino)-5-((tert-butoxycarbonyl)amino)pentanoic acid, HATU, and DIEA in a manner Example 14, Step 2 to provide (1R,3S,5r,7r)-2-(1-(2-(2-((R)-2-(((benzyloxy)carbonyl)amino)-5-((tert-butoxycarbonyl)amino)pentanamido)ethyl)-7-isopropyl-1,2,3,4-tetrahydroisoquinolin-6-yl)-5-(2,6-dimethoxyphenyl)-1H-pyrazole-3-carboxamido)adamantane-2-carboxylic acid (30 mg, 30 μmol, 35%) which was used in the next step without further purification.

Steps 2-4: The product from Step 1 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5, then treated with TFA in a manner similar to Example 13, Step 5 to provide the 2,2,2-trifluoroacetic acid salt of 2,2′,2″-(10-(2-(((R)-4-amino-5-((2-(6-(3-(((1R,3 S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(1H)-yl)ethyl)amino)-5-oxopentyl)amino)-2-oxoethyl)-1,4,7,10-tetraazacyclododecane-1,4,7-triyl)triacetic acid (10.5 mg, 8.36 μmol, 53%) as a white solid. MS: Calc'd for C₅₈H₈₃N₁₁O₁₃: 1141.62, found [M+H]⁺: 1142.5.

### Example 71: Compound 71

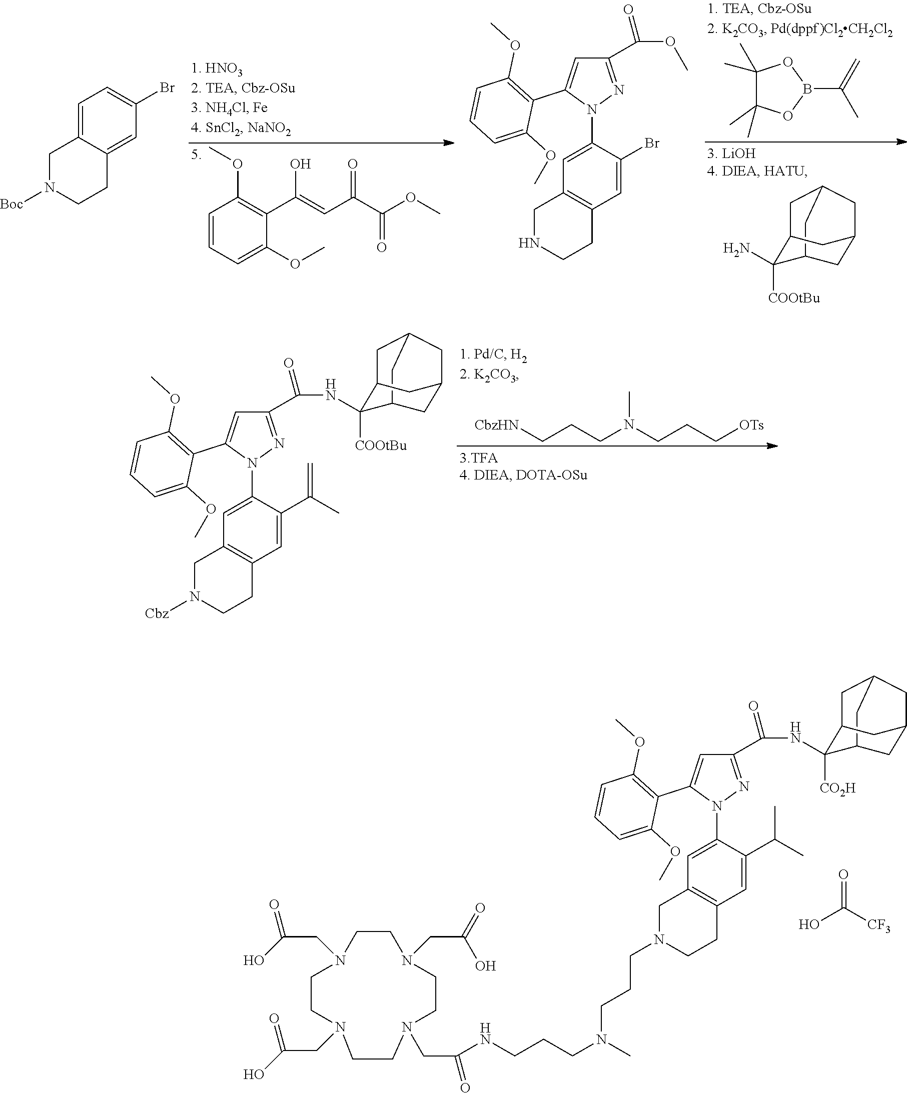

Step 1: tert-Butyl 6-bromo-3,4-dihydroisoquinoline-2(1H)-carboxylate (2.0 g, 1 Eq, 6.4 mmol) was combined with H₂SO₄ (20 mL) at 0° C. for 0.5 hours, then the mixture was added to nitric acid (1.0 g, 60% Wt, 1.5 Eq, 9.5 mmol) dropwise at 0° C. over 0.5 hours. The reaction mixture was stirred at 25° C. for 1 hour. The mixture was directly purified by Prep-HPLC to provide 6-bromo-7-nitro-1,2,3,4-tetrahydroisoquinoline (2.0 g, 5.1 mmol, 79%) as a white solid. [M+H+MeCN]⁺=298.1, 300.1.

Steps 2-9: Benzyl 7-(3-(((1R,3S,5r,7r)-2-(tert-butoxycarbonyl)adamantan-2-yl)carbamoyl)-5-(2,6-dimethoxyphenyl)-1H-pyrazol-1-yl)-6-(prop-1-en-2-yl)-3,4-dihydroisoquinoline-2(1H)-carboxylate was synthesized from 6-bromo-7-nitro-1,2,3,4-tetrahydroisoquinoline in a manner similar to Example 9 to provide the product (177 mg, 0.21 mmol, 67%) as a white solid. [M+H]⁺=787.5.

Step 10: The product from Step 9 was treated with hydrogen and Pd/C in a manner similar to Example 1, Step 7 to provide tert-butyl (1R,3S,5r,7r)-2-(5-(2,6-dimethoxyphenyl)-1-(6-isopropyl-1,2,3,4-tetrahydroisoquinolin-7-yl)-1H-pyrazole-3-carboxamido)adamantane-2-carboxylate (135 mg, 206 μmol, 95.4%) as a white solid. [M+H]⁺=655.4.

Step 11: To a mixture of tert-butyl (1R,3S,5r,7r)-2-(5-(2,6-dimethoxyphenyl)-1-(6-isopropyl-1,2,3,4-tetrahydroisoquinolin-7-yl)-1H-pyrazole-3-carboxamido)adamantane-2-carboxylate (65 mg, 1 Eq, 99 μmol), K₂CO₃ (27 mg, 2.0 Eq, 0.20 mmol) and DMF (1 mL), was added 3-((3-(((benzyloxy)carbonyl)amino)propyl)(methyl)amino)propyl 4-methylbenzenesulfonate (52 mg, 1.2 Eq, 0.12 mmol). The reaction mixture was stirred at 70° C. for 3 hours. The mixture was directly purified by Prep-H PLC to provide tert-butyl (1R,3S,5r,7r)-2-(1-(2-(3-((3-(((benzyloxy)carbonyl)amino)propyl)(methyl)amino)propyl)-6-isopropyl-1,2,3,4-tetrahydroisoquinolin-7-yl)-5-(2,6-dimethoxyphenyl)-1H-pyrazole-3-carboxamido)adamantane-2-carboxylate (60 mg, 62 μmol, 63%) as a white solid. [M+1H]⁺=917.5.

Steps 12-13: The product from Step 11 was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((3-((3-(7-(3-(((1R,3S,5r,7r)-2-carboxyadamantan-2-yl)carbamoyl)-5-(2,6-dimethoxyphenyl)-1H-pyrazol-1-yl)-6-isopropyl-3,4-dihydroisoquinolin-2(1H)-yl)propyl)(methyl)amino)propyl)amino)-2-oxoethyl)-1,4,7,10-tetraazacyclododecane-1,4,7-triyl)triacetic acid (24.2 mg, 18.1 μmol, 24%) as a white solid. MS: Calc'd for C₅₈H₈₄N₁₀O₁₂: 1112.63, found [M+H]⁺: 1113.6.

### Example 72: Compound 72

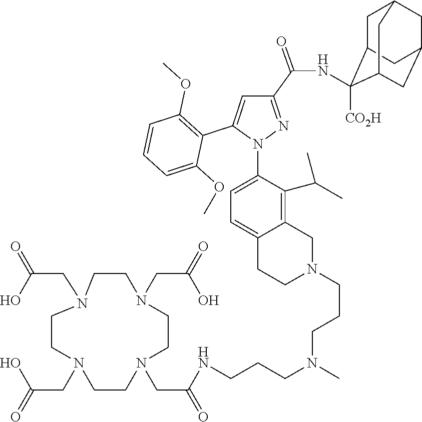 [SMILES: COc1cccc(OC)c1-c1cc(C(=O)NC2(C(=O)O)C3CC4CC(C3)CC2C4)nn1-c1ccc2c(c1C(C)C)CN(CCCN(C)CCCNC(=O)CN1CCN(CC(=O)O)CCN(CC(=O)O)CCN(CC(=O)O)CC1)CC2]

### Example 73: Compound 73

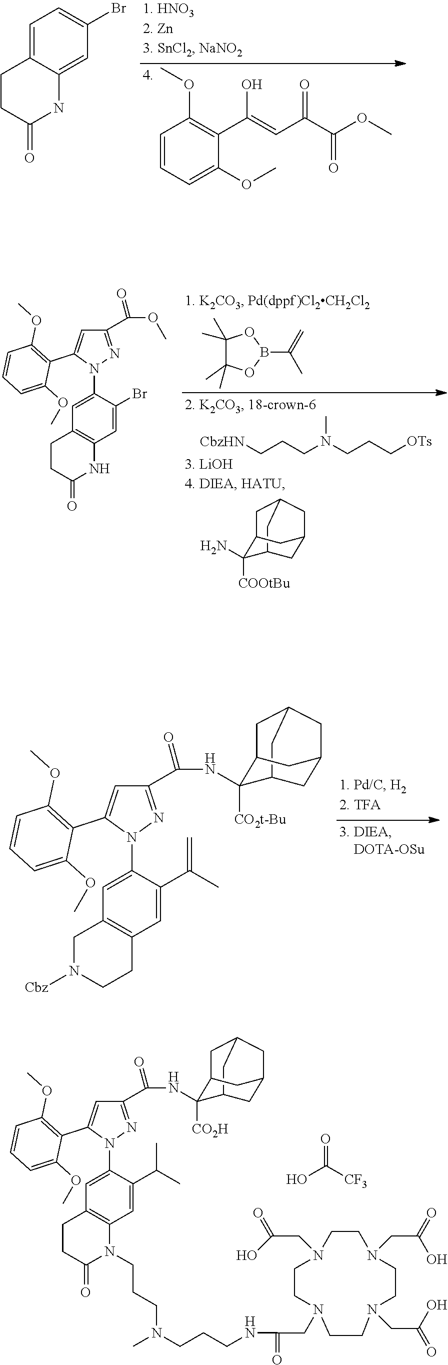

Step 1: 7-bromo-6-nitro-3,4-dihydroquinolin-2(1-1)-one was synthesized from 7-bromo-3,4-dihydroquinolin-2(1H)-one in a manner similar to Example 71, Step 1, to provide the product (1.2 g, 4.4 mmol, 50%) as a yellow crude solid.

Step 2: Into a 40-mL vial, was placed a mixture of 7-bromo-6-nitro-3,4-dihydroquinolin-2(1-1)-one (1.2 g, 1 Eq, 4.4 mmol) and AcOH (15 mL), to which was added zinc (2.0 g, 6.9 Eq, 31 mmol). The reaction mixture was stirred at 25° C. for 2 hours. The mixture filtered then the supernatant was concentrated under reduced pressure. The resulting solid was washed twice with DCM, to afford 6-amino-7-bromo-3,4-dihydroquinolin-2(1H)-one (900 mg, 3.73 mmol, 84%) as a brown crude solid. [M+H]⁺=238.9, 240.9.

Steps 3-4: Methyl 1-(7-bromo-2-oxo-1,2,3,4-tetrahydroquinolin-6-yl)-5-(2,6-dimethoxyphenyl)-1H-pyrazole-3-carboxylate was synthesized from 6-amino-7-bromo-3,4-dihydroquinolin-2(1H)-one in a manner similar to Example 9, Steps 3-4, to provide the product (800 mg, 1.65 mmol, 56.9%) as a brown solid. [M+H]⁺=486.0, 488.0.

Step 5: The product from Step 4 was treated with 4,4,5,5-tetramethyl-2-(prop-1-en-2-yl)-1,3,2-dioxaborolane, Pd(dtbpf)Cl₂, and K₂CO₃, in a manner similar to Example 71, Step 7 to provide methyl 5-(2,6-dimethoxyphenyl)-1-(2-oxo-7-(prop-1-en-2-yl)-1,2,3,4-tetrahydroquinolin-6-yl)-1H-pyrazole-3-carboxylate (335 mg, 749 μmol, 45.5%) as a yellow solid. [M+H]⁺=448.2.

Step 6: To a mixture of methyl 5-(2,6-dimethoxyphenyl)-1-(2-oxo-7-(prop-1-en-2-yl)-1,2,3,4-tetrahydroquinolin-6-yl)-1H-pyrazole-3-carboxylate (175 mg, 1 Eq, 391 μmol), 18-crown-6 (517 mg, 5.00 Eq, 1.96 mmol), K₂CO₃ (162 mg, 3.00 Eq, 1.17 mmol), 3-((3-(((benzyloxy)carbonyl)amino)propyl)(methyl)amino)propyl 4-methylbenzenesulfonate (170 mg, 1.00 Eq, 391 mol) was added DMF (5 mL). The reaction mixture was stirred at 60° C. for 16 hours. The crude product was purified by Prep-HPLC to afford methyl 1-(1-(3-((3-(((benzyloxy)carbonyl)amino)propyl)(methyl)amino)propyl)-2-oxo-7-(prop-1-en-2-yl)-1,2,3,4-tetrahydroquinolin-6-yl)-5-(2,6-dimethoxyphenyl)-1H-pyrazole-3-carboxylate (50 mg, 70 μmol, 18%) as a yellow solid. [M+H]⁺=710.6.

Steps 7-8: The product from Step 6 was treated with LiOH, then tert-butyl (1R,3 S,5r,7r)-2-aminoadamantane-2-carboxylate, HATU, and DIEA in a manner similar to Example 9, Steps 6-7 to provide tert-butyl (1R,3S,5r,7r)-2-(1-(1-(3-((3-(((benzyloxy)carbonyl)amino)propyl)(methyl)amino)propyl)-2-oxo-7-(prop-1-en-2-yl)-1,2,3,4-tetrahydroquinolin-6-yl)-5-(2,6-dimethoxyphenyl)-1H-pyrazole-3-carboxamido)adamantane-2-carboxylate (22 mg, 24 μmol, 66%) as a yellow oil. [M+H]⁺=929.5.

Steps 9-11: The product from Step 8 was treated with hydrogen and Pd/C in a manner similar to Example 1, Step 7 then treated with TFA, then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″(10-(2-((3-((3-(6-(3-(((1R,3S,5r,7r)-2-carboxyadamantan-2-yl)carbamoyl)-5-(2,6-dimethoxyphenyl)-1H-pyrazol-1-yl)-7-isopropyl-2-oxo-3,4-dihydroquinolin-1(21)-yl)propyl)(methyl)amino)propyl)amino)-2-oxoethyl)-1,4,7,10-tetraazacyclododecane-1,4,7-triyl)triacetic acid (7.5 mg, 6.0 μmol, 35%) as a white solid. MS: Calc'd for C₅₈H₈₂N₁₀O₁₃: 1126.61, found [M+H]⁺: 1127.5.

### Example 74: Compound 74

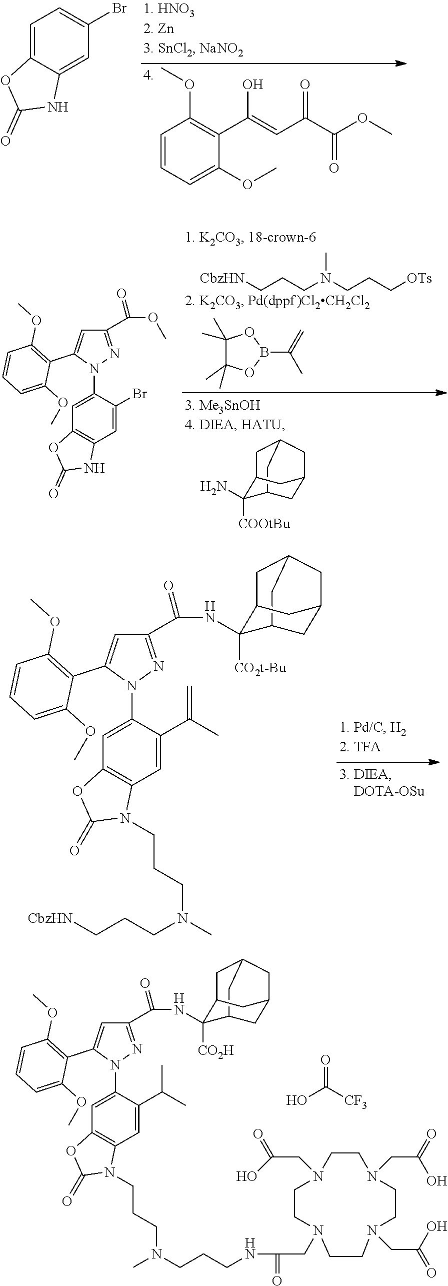

Step 1: 2,2,2″-(10-(2-((3-((3-(6-(3-(((1R,3S,5r,7r)-2-carboxyadamantan-2-yl)carbamoyl)-5-(2,6-dimethoxyphenyl)-1H-pyrazol-1-yl)-5-isopropyl-2-oxobenzo[d]oxazol-3(2H)-yl)propyl)(methyl)amino)propyl)amino)-2-oxoethyl)-1,4,7,10-tetraazacyclododecane-1,4,7-triyl)triacetic acid was synthesized from 5-bromobenzo[d]oxazol-2(3-1)-one in a manner similar to Example 73 to provide the 2,2,2-trifluoroacetic acid salt of Compound 74 (11.3 mg, 9.19 μmol, 34%) as a white solid. MS: Calc'd for C₅₆H₇₈N₁₀O₁₄: 1114.57, found [M+H]⁺: 1115.5.

### Example 75: Compound 75

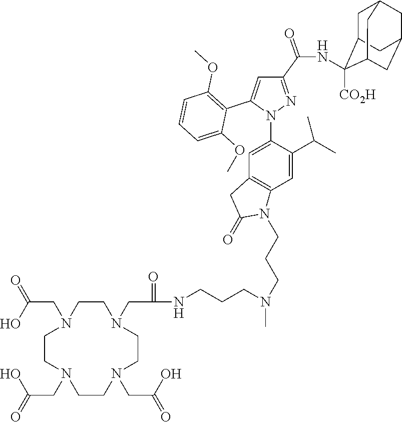 [SMILES: COc1cccc(OC)c1-c1cc(C(=O)NC2(C(=O)O)C3CC4CC(C3)CC2C4)nn1-c1cc2c(cc1C(C)C)N(CCCN(C)CCCNC(=O)CN1CCN(CC(=O)O)CCN(CC(=O)O)CCN(CC(=O)O)CC1)C(=O)C2]

### Example 76: Compound 76

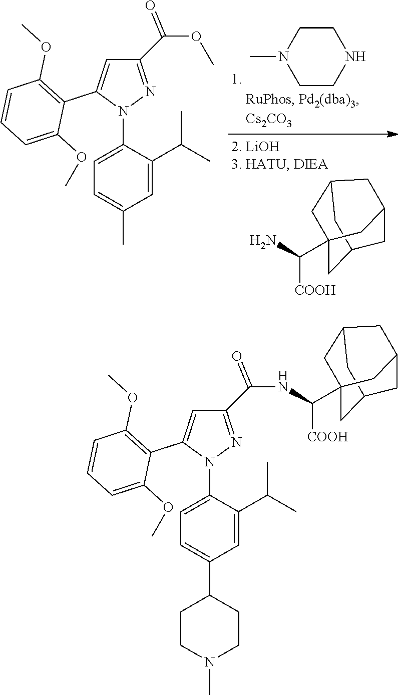

Step 1: Methyl 5-(2,6-dimethoxyphenyl)-1-(4-iodo-2-isopropylphenyl)-1H-pyrazole-3-carboxylate (from Example 1, Step 3) (200 mg, 1 Eq, 395 μmol) was treated with 2-Dicyclohexylphosphino-2′,6′-diisopropoxybiphenyl (276 mg, 1.50 Eq, 591 μmol), 1-methylpiperazine (8 mg, 0.2 Eq, 0.08 mmol), Pd₂(dba)₃ (36 mg, 0.10 Eq, 39 μmol), Cs₂CO₃ (386 mg, 3.00 Eq, 1.18 mmol) and 1,4-dioxane (5 mL). The reaction mixture was stirred at 100° C. for 1 hour under N₂. The mixture was directly purified by MPLC to afford methyl 5-(2,6-dimethoxyphenyl)-1-(2-isopropyl-4-(4-methylpiperazin-1-yl)phenyl)-1-pyrazole-3-carboxylate (130 mg, 272 μmol, 68.8%) as a yellow solid. [M+H]⁺=479.3.

Step 2: Into a 8-mL vial, was placed a mixture of methyl 5-(2,6-dimethoxyphenyl)-1-(2-isopropyl-4-(4-methylpiperazin-1-yl)phenyl)-1H-pyrazole-3-carboxylate (130 mg, 1 Eq, 272 μmol), MeOH (3 mL) and water (1 mL), to which was added LiOH (33 mg, 5.1 Eq, 1.4 mmol). The reaction mixture was stirred at 25° C. for 16 hours. The mixture was concentrated under reduced pressure. The crude product was purified by Prep-HPLC and the collected fractions were dried by lyophilization to afford in 5-(2,6-dimethoxyphenyl)-1-(2-isopropyl-4-(4-methylpiperazin-1-yl)phenyl)-1H-pyrazole-3-carboxylic acid (110 mg, 237 μmol, 87.2%) as a white solid.

Step 3: Into an 8-mL vial, was placed a mixture of 5-(2,6-dimethoxyphenyl)-1-(2-isopropyl-4-(4-methylpiperazin-1-yl)phenyl)-1H-pyrazole-3-carboxylic acid (20 mg, 1 Eq, 43 μmol), HATU (25 mg, 1.5 Eq, 66 μmol), DIEA (17 mg, 23 μL, 3.1 Eq, 0.13 mmol) and DMF (2 mL). The reaction mixture was stirred at 25° C. for 10 minutes, then (2S)-2-((3R,5R)-adamantan-1-yl)-2-aminoacetic acid (10 mg, 1.1 Eq, 48 μmol) was added and the reaction mixture was stirred at 25° C. for an additional 1 hour. The crude product was purified by Prep-HPLC and the collected fractions were dried by lyophilization to afford the trifluoroacetic acid salt of (2S)-2-((3R,5R)-adamantan-1-yl)-2-(5-(2,6-dimethoxyphenyl)-1-(2-isopropyl-4-(4-methylpiperazin-1-yl)phenyl)-1H-pyrazole-3-carboxamido)acetic acid (8.3 mg, 11 μmol, 25%) as a white solid. MS: Calc'd for C₄₀H₅₀F₃N₅O₇: 655.37, found [M+H]⁺: 656.4.

### Example 77: Compound 77

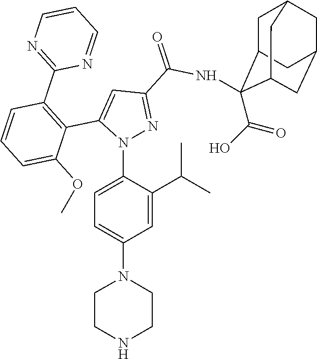 [SMILES: COc1cccc(-c2ncccn2)c1-c1cc(C(=O)NC2(C(=O)O)C3CC4CC(C3)CC2C4)nn1-c1ccc(N2CCNCC2)cc1C(C)C]

### Example 78: Compound 78

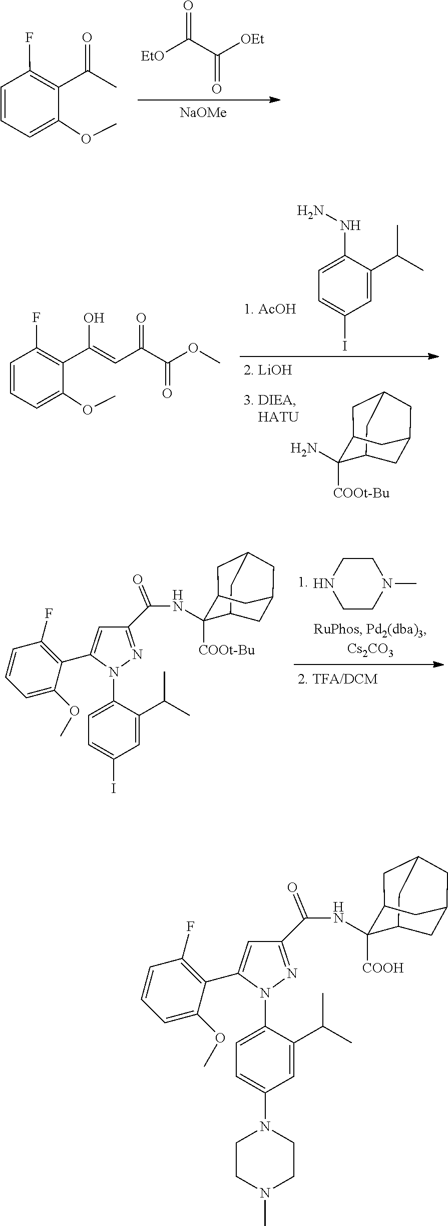

Step 1: A mixture of 1-(2-fluoro-6-methoxyphenyl)ethan-1-one (1.2 g, 1 Eq, 7.1 mmol), diethyl oxalate (1.1 g, 1.1 Eq, 7.5 mmol), sodium methoxide (0.79 g, 0.85 mL, 2.0 Eq, 15 mmol), and 1,4-dioxane (2 mL) was stirred at 70° C. for 16 hours. The mixture was directly purified by MPLC to afford methyl (Z)-4-(2-fluoro-6-methoxyphenyl)-4-hydroxy-2-oxobut-3-enoate (900 mg, 3.54 mmol, 50%) as a yellow solid. [M+H]⁺=255.0.

Steps 2-4: tert-Butyl (1R,3S,5r,7r)-2-(5-(2-fluoro-6-methoxyphenyl)-1-(4-iodo-2-isopropylphenyl)-1H-pyrazole-3-carboxamido)ad amantane-2-carboxylate was prepared from methyl (Z)-4-(2-fluoro-6-methoxyphenyl)-4-hydroxy-2-oxobut-3-enoate and (4-iodo-2-isopropylphenyl)hydrazine in a manner similar to Example 1, Steps 3-5 to provide the product (40 mg, 56 μmol, 30%) as a white solid. [M+H]⁺=714.2.

Steps 5-6: (1R,3S,5r,7r)-2-(5-(2-fluoro-6-methoxyphenyl)-1-(2-isopropyl-4-(4-methylpiperazin-1-yl)phenyl)-1H-pyrazole-3-carboxamido)adamantane-2-carboxylic acid was synthesized from methyl (Z)-4-(2-fluoro-6-methoxyphenyl)-4-hydroxy-2-oxobut-3-enoate and (4-iodo-2-isopropylphenyl)hydrazine and 1-methylpiperazine in a manner similar to Example 6 to provide the 2,2,2-trifluoroacetic acid of the product (10.8 mg, 14.5 μmol, 50%) as a white solid. MS: Calc'd for C₃₆H₄₄FN₅O₄: 629.34, found [M+H]⁺: 630.5.

### Example 79: Compound 79

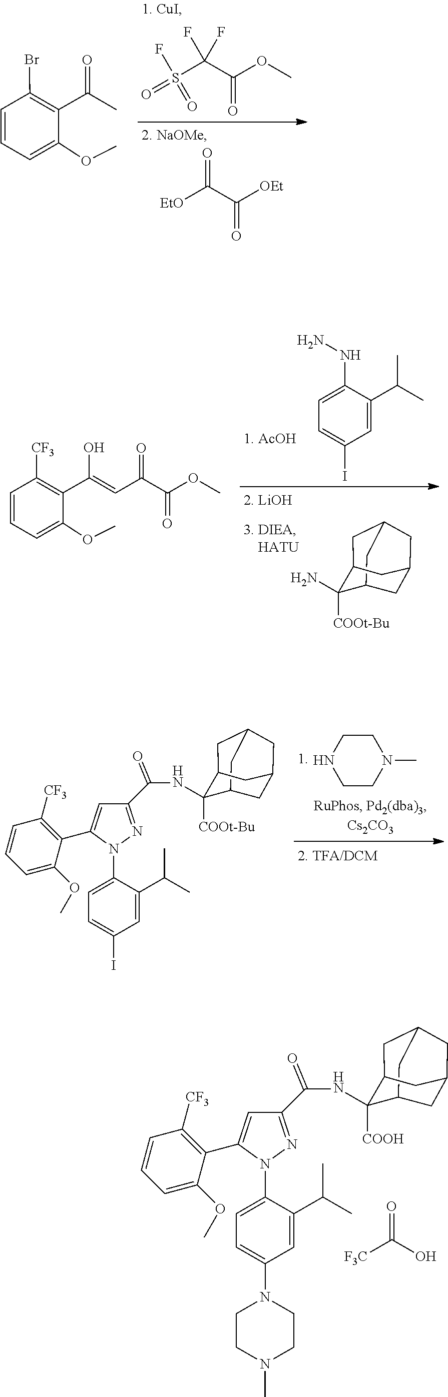

Step 1: To a mixture of 1-(2-bromo-6-methoxyphenyl)ethan-1-one (2.2 g, 1 Eq, 9.6 mmol) in DMF (25 mL) was added CuI (3.7 g, 2.0 Eq, 19 mmol), then methyl 2,2-difluoro-2-(fluorosulfonyl)acetate (2.2 g, 1.2 Eq, 11 mmol) was added dropwise at 25° C. The reaction mixture was then stirred at 110° C. for 16 hour. The reaction solution was allowed to cool to room temperature and the mixture was directly purified by Prep-HPLC to afford 1-(2-methoxy-6-(trifluoromethyl)phenyl)ethan-1-one (300 mg, 1.38 mmol, 14%) as a white solid. [M+H+MeCN]⁺=260.1.

Steps 2-6: (1R,3 S,5r,7r)-2-(1-(2-isopropyl-4-(4-methylpiperazin-1-yl)phenyl)-5-(2-methoxy-6-(trifluoromethyl)phenyl)-1H-pyrazole-3-carboxamido)adamantane-2-carboxylic acid was synthesized from 1-(2-methoxy-6-(trifluoromethyl)phenyl)ethan-1-one (180 mg, 1 Eq, 825 μmol) in a manner similar to Example 78, Steps 1-6 to afford the 2,2,2-trifluoroacetic acid salt of the product (1.3 mg, 1.6 μmol, 40%) as a white solid. MS: Calc'd for C₃₇H₄₄F₃N₅O₄: 679.33, found [M+H]⁺: 680.4.

### Example 80: Compound 80

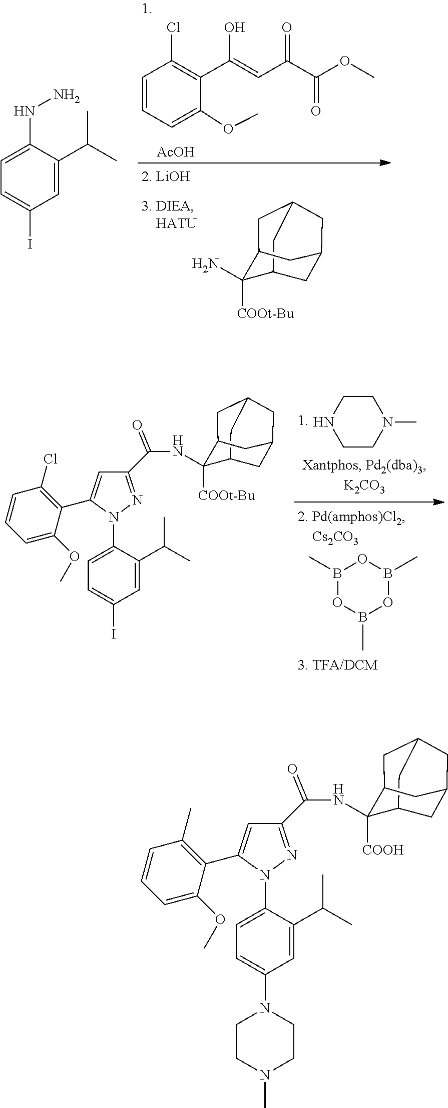

Steps 1-3: tert-Butyl (1R,3S,5r,7r)-2-(5-(2-chloro-6-methoxyphenyl)-1-(4-iodo-2-isopropylphenyl)-1H-pyrazole-3-carboxamido)adamantane-2-carboxylate was synthesized from (4-iodo-2-isopropylphenyl)hydrazine in a manner similar to Steps 3-5 of Example 1 to provide the product (1.04 g, 1.42 mmol, 71%) as a yellow solid.

Step 4: Into a 40-mL vial purged and maintained with an inert atmosphere of nitrogen, was placed with a mixture of tert-butyl (1R,3S,5r,7r)-2-(5-(2-chloro-6-methoxyphenyl)-1-(4-iodo-2-isopropylphenyl)-1H-pyrazole-3-carboxamido)adamantane-2-carboxylate(190 mg, 1 Eq, 260 μmol), tris(dibenzylideneacetone) dipalladium(0) (23.9 mg, 0.100 Eq, 26.1 μmol), 4,5-bis(diphenylphosphino)-9,9-dimethyl xanthene (30.1 mg, 0.200 Eq, 52.0 μmol), 1-methylpiperazine (130 mg, 4.99 Eq, 1.30 mmol), K₂CO₃ (108 mg, 3.00 Eq, 781 μmol) and toluene (5 mL). The reaction mixture was stirred at 100° C. for 1 hour under N₂. The mixture was directly purified by MPLC and the collected fractions were concentrated to afford tert-butyl (1R,3 S,5r,7r)-2-(5-(2-chloro-6-methoxyphenyl)-1-(2-isopropyl-4-(4-methylpiperazin-1-yl)phenyl)-1H-pyrazole-3-carboxamido)adamantane-2-carboxylate (150 mg, 214 μmol, 82.1%) as a yellow solid. [M+H]⁺=702.6.

Step 5: Into a 40-mL vial purged and maintained with an inert atmosphere of nitrogen, was placed with a mixture of tert-butyl (1R,3S,5r,7r)-2-(5-(2-chloro-6-methoxyphenyl)-1-(2-isopropyl-4-(4-methylpiperazin-1-yl)phenyl)-1H-pyrazole-3-carboxamido)adamantane-2-carboxylate (50 mg, 1 Eq, 71 mol), bis(di-tert-butyl (4-dimethylaminophenyl)phosphine)dichloropalladium(II) (10 mg, 0.20 Eq, 14 μmol), 2,4,6-trimethyl-1,3,5,2,4,6-trioxatriborinane (0.18 g, 20 Eq, 1.4 mmol), cesium carbonate (70 mg, 17 μL, 3.0 Eq, 0.21 mmol) and 1,4-dioxane (3 mL). The reaction mixture was stirred at 120° C. for 2 hours under N₂. The mixture was directly purified by MPLC and the collected fractions were concentrated to afford tert-butyl (1R,3S,5r,7r)-2-(1-(2-isopropyl-4-(4-methylpiperazin-1-yl)phenyl)-5-(2-methoxy-6-methylphenyl)-1H-pyrazole-3-carboxamido)adamantane-2-carboxylate (30 mg, 44 μmol, 62%) as a yellow solid.

Step 6: tert-butyl (1R,3S,5r,7r)-2-(1-(2-isopropyl-4-(4-methylpiperazin-1-yl)phenyl)-5-(2-methoxy-6-methylphenyl)-1H-pyrazole-3-carboxamido)adamantane-2-carboxylate (30 mg, 1 Eq, 44 μmol) was treated with TFA (0.6 mL) and DCM (2 mL), and the reaction mixture was stirred at 25° C. for 1 hour. The mixture was concentrated under reduced pressure and the crude product was purified by Prep-HPLC to afford the trifluoroacetic acid salt of (1R,3S,5r,7r)-2-(1-(2-isopropyl-4-(4-methylpiperazin-1-yl)phenyl)-5-(2-methoxy-6-methylphenyl)-1H-pyrazole-3-carboxamido)adamantane-2-carboxylic acid (12.0 mg, 16.2 μmol, 37%) as a white solid. MS: Calc'd for C₃₉H₄F₃N₅O₆: 625.36, found [M+H]⁺: 626.4.

### Example 81: Compound 81

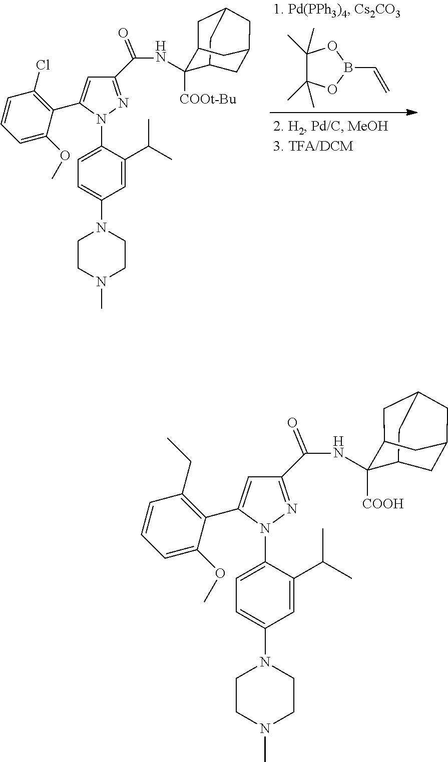

Step 1: Into a 40-mL vial purged and maintained with an inert atmosphere of nitrogen, was placed with a mixture of tert-butyl (1R,3S,5r,7r)-2-(5-(2-chloro-6-methoxyphenyl)-1-(2-isopropyl-4-(4-methylpiperazin-1-yl)phenyl)-1H-pyrazole-3-carboxamido)adamantane-2-carboxylate (50 mg, 1 Eq, 71 μmol), tetrakis(triphenylphosphine)palladium(0) (16 mg, 0.19 Eq, 14 μmol), 4,4,5,5-tetramethyl-2-vinyl-1,3,2-dioxaborolane (0.11 g, 10 Eq, 0.71 mmol), Cs₂CO₃ (23 mg, 0.99 Eq, 71 mol) and dioxane (3 mL). The reaction mixture was stirred at 25° C. for 1 hour under N₂. The mixture was directly purified by MPLC to afford tert-butyl (1R,3 S,5r,7r)-2-(1-(2-isopropyl-4-(4-methylpiperazin-1-yl)phenyl)-5-(2-methoxy-6-vinylphenyl)-1H-pyrazole-3-carboxamido)adamantane-2-carboxylate (30 mg, 43 μmol, 61%) as a yellow solid.

Step 2: tert-butyl (1R,3 S,5r,7r)-2-(1-(2-isopropyl-4-(4-methylpiperazin-1-yl)phenyl)-5-(2-methoxy-6-vinylphenyl)-1H-pyrazole-3-carboxamido)adamantane-2-carboxylate was treated with H₂ and Pd/C and MeOH in a manner similar to Example 1, Step 7 to provide tert-butyl (1R,3 S,5r,7r)-2-(5-(2-ethyl-6-methoxyphenyl)-1-(2-isopropyl-4-(4-methylpiperazin-1-yl)phenyl)-1H-pyrazole-3-carboxamido)adamantane-2-carboxylate (25 mg, 36 μmol, 83%) as a white solid.

Step 3: tert-butyl (1R,3S,5r,7r)-2-(5-(2-ethyl-6-methoxyphenyl)-1-(2-isopropyl-4-(4-methylpiperazin-1-yl)phenyl)-1H-pyrazole-3-carboxamido)adamantane-2-carboxylate is treated with TFA/DCM in a manner similar to Example 1, Step 8 to provide the trifluoroacetic acid salt of (1R,3S,5r,7r)-2-(5-(2-ethyl-6-methoxyphenyl)-1-(2-isopropyl-4-(4-methylpiperazin-1-yl)phenyl)-1H-pyrazole-3-carboxamido)adamantane-2-carboxylic acid as a white solid. MS: Calc'd for C₃₈H₅₀N₅O₄: 640.39, found [M+H]⁺: 640.4.

### Example 82: Compound 82

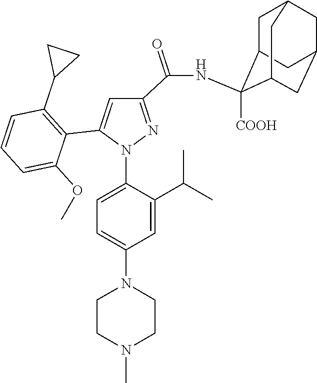 [SMILES: COc1cccc(C2CC2)c1-c1cc(C(=O)NC2(C(=O)O)C3CC4CC(C3)CC2C4)nn1-c1ccc(N2CCN(C)CC2)cc1C(C)C]

### Example 83: Compound 83

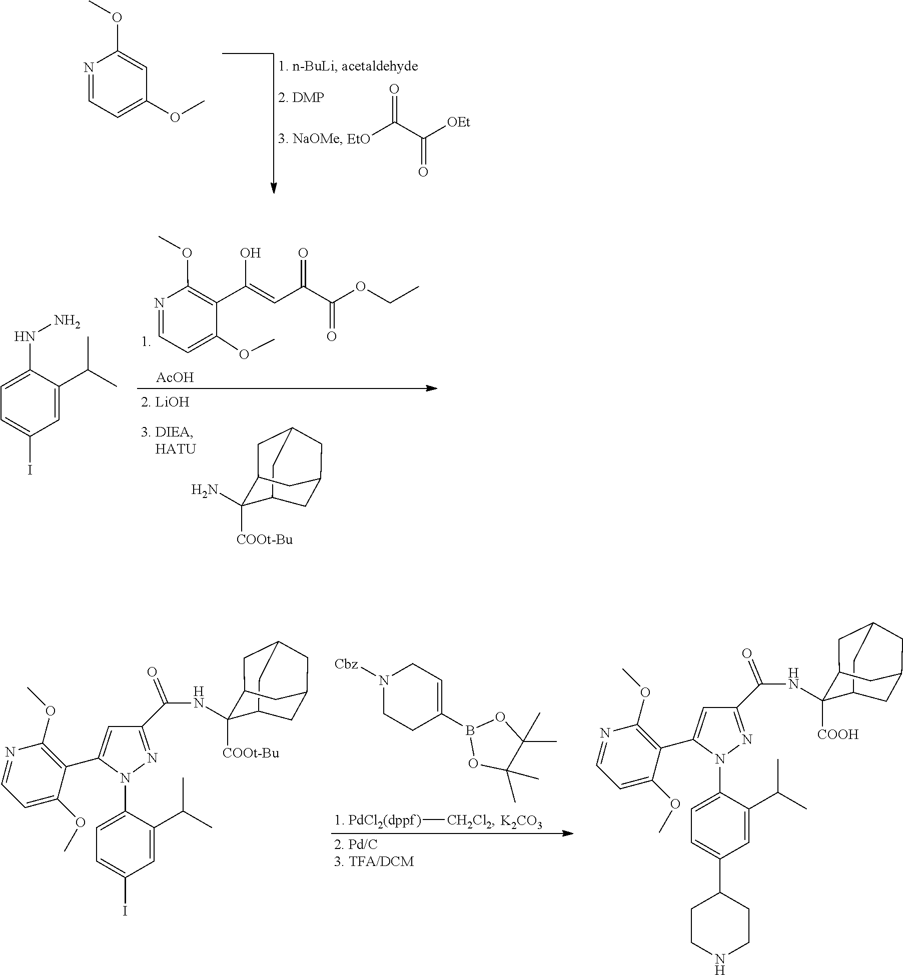

Step 1: To a mixture of 2,4-dimethoxypyridine (12.0 g, 1 Eq, 86.2 mmol) and THE (130 mL) was added n-butyllithium (6.08 g, 47.5 mL, 2 molar, 1.10 Eq, 94.9 mmol) dropwise at −78° C. over 10 min. The reaction mixture was stirred at −78° C. for 1 hour then acetaldehyde (4.18 g, 1.10 Eq, 94.9 mmol) was added dropwise at −78° C. over 10 min. the reaction mixture was stirred at −78° C. for additional 3 hours. The mixture was quenched with an aqueous solution of NH₄Cl (50 mL), extracted with EtOAc (50 mL×3), then the combined organic layers were washed with brine (50 mL), dried over anhydrous Na₂SO₄ and concentrated under reduced pressure. The crude product was purified by MPLC to afford 1-(2,4-dimethoxypyridin-3-yl)ethan-1-ol (14.4 g, 78.6 mmol, 91.1%) as a yellow solid.

Step 2: To a mixture of 1-(2,4-dimethoxypyridin-3-yl)ethan-1-ol (12.7 g, 1 Eq, 69.3 mmol) and DCM (150 mL) was added dess-martin periodinane (35.3 g, 1.20 Eq, 83.2 mmol). The reaction mixture was stirred at 25° C. for 1 hour. The mixture was concentrated and the crude product was purified by MPLC to provide 1-(2,4-dimethoxypyridin-3-yl)ethan-1-one (6.5 g, 36 mmol, 52%) as a yellow oil.

Step 3: A mixture of 1-(2,4-dimethoxypyridin-3-yl)ethan-1-one (2.3 g, 1 Eq, 13 mmol), diethyl oxalate (2.2 g, 1.2 Eq, 15 mmol), sodium methanolate (3.4 g, 5.0 Eq, 63 mmol) and dioxane (30 mL) was stirred at 25° C. for 16 hours. The mixture was concentrated and the crude product was purified by MPLC to afford ethyl (Z)-4-(2,4-dimethoxypyridin-3-yl)-4-hydroxy-2-oxobut-3-enoate (3.7 g, 11 mmol, 83%) as a yellow solid. [M+H]⁺=282.1

Steps 4-9: The trifluoroacetic acid salt of (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylic acid (10.9 mg, 14.7 μmol, 67%) was synthesized from ethyl (Z)-4-(2,4-dimethoxypyridin-3-yl)-4-hydroxy-2-oxobut-3-enoate and (4-iodo-2-isopropylphenyl)hydrazine in a manner similar to steps 3-8 of Example 1 to provide the product (10.9 mg, 14.7 μmol, 67%). MS: Calc d for C₃₆H₄₆N₅O₅: 628.35, found [M+H]⁺: 628.4.

### Example 84: Compound 84

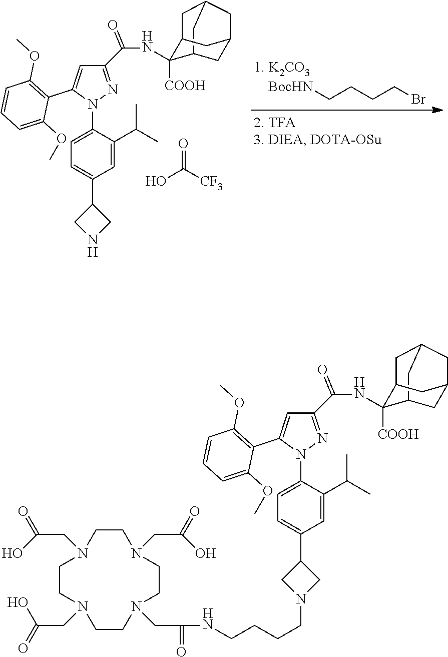

Step 1. A mixture of tert-butyl (1R,3S,5r,7r)-2-(1-(4-(azetidin-3-yl)-2-isopropylphenyl)-5-(2,6-dimethoxyphenyl)-1H-pyrazole-3-carboxamido)adamantane-2-carboxylate (70 mg, 1 Eq, 0.11 mmol), tert-butyl (4-bromobutyl)carbamate (54 mg, 2.0 Eq, 0.21 mmol), K₂CO₃ (44 mg, 3.0 Eq, 0.32 mmol), and DMF (2 mL) was stirred at 80° C. for 2 hours. The mixture was concentrated under reduced pressure. The crude product was purified by Prep-HPLC to afford tert-butyl (1R,3S,5r,7r)-2-(1-(4-(1(4-(((benzyloxy)carbonyl)amino)butyl)azetidin-3-yl)-2-isopropylphenyl)-5-(2,6-dimethoxyphenyl)-1H-pyrazole-3-carboxamido)adamantane-2-carboxylate (60 mug, 70 μmol, 65%) as a white solid.

Steps 2-3: tert-Butyl (1R,3S,5r,7r)-2-(i-(4-(i-(4-(((benzyloxy)carbonyl)amino)butyl)azetidin-3-yl)-2-isopropylphenyl)-5-(2,6-dimethoxyphenyl)-1H-pyrazole-3-carboxamido)adamantane-2-carboxylate was treated with TFA, then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid, in a manner similar to Example 13, Steps 4 and 5 to provide the 2,2,2-trifluoroacetic acid salt of 2,2′,2″-(10-(2-((4-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)azetidin-1-yl)butyl)amino)-2-oxoethyl)-1,4,7,10-tetraazacyclododecane-1,4,7-triyl)triacetic acid (37.0 mg, 31.6 μmol, 42%) as a white solid. MS: Calc'd for C₅₇H₇₇N₉O₁₂: 1055.57, found [M+H]⁺: 1056.5.

### Example 85: Compound 85

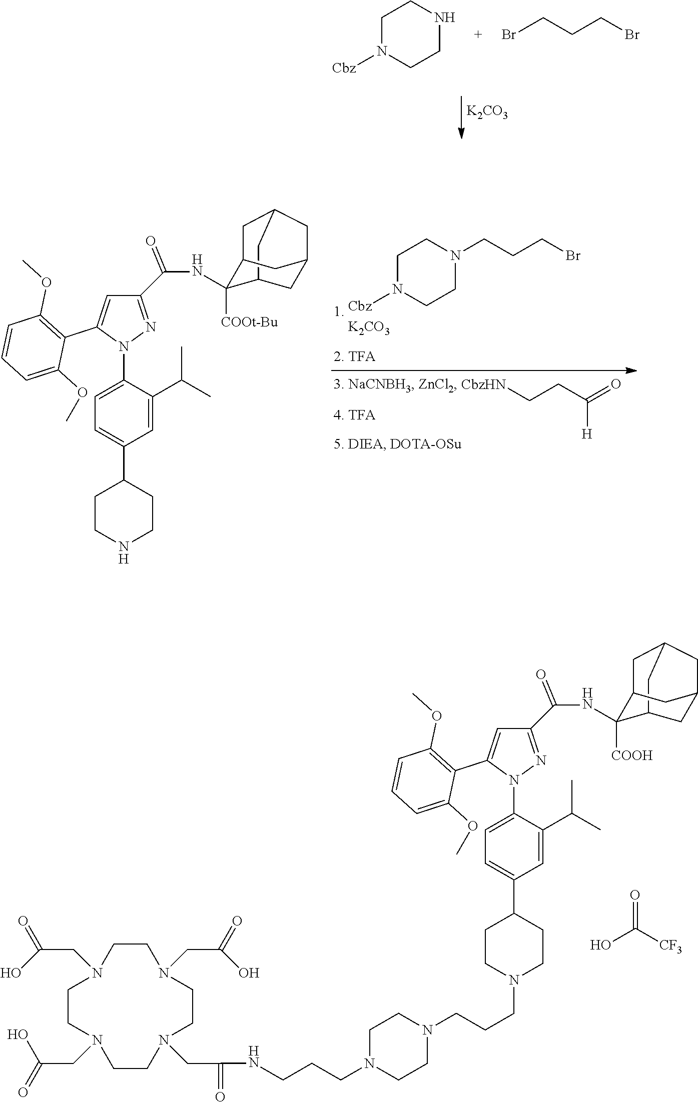

Step 1: To a mixture of 1,3-dibromopropane (500 mg, 1 Eq, 2.48 mmol), benzyl piperazine-1-carboxylate (273 mg, 0.500 Eq, 1.24 mmol) and ACN (5 mL), was added K₂CO₃ (1.03 g, 3.01 Eq, 7.45 mmol). The reaction mixture was stirred at 80° C. for 1 hour. The crude mixture was directly purified by Prep-HPLC to provide benzyl 4-(3-bromopropyl)piperazine-1-carboxylate (120 mg, 352 mol, 14.2%) as a white oil.

Step 2: To a mixture of (1R,3S,5r,7r)-2-(5-(2,6-dimethoxyphenyl)-1-(2-isopropyl-4-(piperidin-4-yl)phenyl)-1H-pyrazole-3-carboxamido)adamantan-2-yl pivalate (145 mg, 1 Eq, 212 μmol), benzyl 4-(3-bromopropyl)piperazine-1-carboxylate (217 mg, 2.99 Eq, 636 μmol) and ACN (5 mL) was added potassium carbonate (88.0 mg, 3.00 Eq, 637 mol). The reaction mixture was stirred at 80° C. for 1 hour. The crude mixture was directly purified by Prep-HPLC to provide benzyl 4-(3-(4-(4-(5-(2,6-dimethoxyphenyl)-3-(((1R,3S,5r,7r)-2-(pivaloyloxy)adamantan-2-yl)carbamoyl)-1H-pyrazol-1-yl)-3-isopropylphenyl)piperidin-1-yl)propyl)piperazine-1-carboxylate (130 mg, 138 μmol, 64.9%) as a white solid. [M+H]⁺=943.7.

Step 3: benzyl 4-(3-(4-(4-(5-(2,6-dimethoxyphenyl)-3-(((1R,3S,5r,7r)-2-(pivaloyloxy)adamantan-2-yl)carbamoyl)-1H-pyrazol-1-yl)-3-isopropylphenyl)piperidin-1-yl)propyl)piperazine-1-carboxylate was treated with TFA in a manner similar to Example 1, Step 8 to provide (1R,3S,5r,7r)-2-(5-(2,6-dimethoxyphenyl)-1-(2-isopropyl-4-(1-(3-(piperazin-1-yl)propyl)piperidin-4-yl)phenyl)-1H-pyrazole-3-carboxamido)adamantane-2-carboxylic acid (90 mg, 0.12 mmol, 87%) as a white solid.

Step 4: A mixture of (1R,3 S,5r,7r)-2-(5-(2,6-dimethoxyphenyl)-1-(2-isopropyl-4-(1-(3-(piperazin-1-yl)propyl)piperidin-4-yl)phenyl)-1H-pyrazole-3-carboxamido)adamantane-2-carboxylic acid (90 mg, 1 Eq, 0.12 mmol), benzyl (3-oxopropyl)carbamate (25 mg, 1 Eq, 0.12 mmol), zinc chloride (49 mg, 23 μL, 3 Eq, 0.36 mmol) and MeOH (3 mL) was stirred at 25° C. for 10 minutes, then sodium cyanoborohydride (15 mg, 14 μL, 2 Eq, 0.24 mmol) was added and the reaction mixture was stirred at 25° C. for 2 h. The mixture was directly purified by MPLC to afford (1R,3S,5r,7r)-2-(1-(4-(1-(3-(4-(3-(((benzyloxy)carbonyl)amino)propyl)piperazin-1l-yl)propyl)piperidin-4-yl)-2-isopropylphenyl)-5-(2,6-dimethoxyphenyl)-1H-pyrazole-3-carboxamido)adamantane-2-carboxylic acid (70 mg, 74 μmol, 62%) as a white solid. [M+H]⁺=944.7.

Steps 5-6: (1R,3S,5r,7r)-2-(1-(4-(1-(3-(4-(3-(((benzyloxy)carbonyl)amino)propyl)piperazin-1-yl)propyl)piperidin-4-yl)-2-isopropylphenyl)-5-(2,6-dimethoxyphenyl)-1H-pyrazole-3-carboxamido)adamantane-2-carboxylic acid was treated with TEA, then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid, in a manner similar to Example 13, Steps 4 and 5 to provide the 2,2,2-trifluoroacetic acid salt of 2,2′,2″-(10-(2-((3-(4-(3-(4-(4-(3-(((1R,3S,5r,7r)-2-carboxyadamantan-2-yl)carbamoyl)-5-(2,6-dimethoxyphenyl)-11H-pyrazol-¹-yl)-³-isopropylphenyl)piperidin-1-yl)propyl)piperazin-1-yl)propyl)amino)-2-oxoethyl)-1,4,7,10-tetraazacyclododecane-1,4,7-triyl)triacetic acid (8.6 mg, 6.6 mol, 19%) as a white solid. MS: Calc'd for C₆H₉₄F₁₃N₁₀O₁₄: 1195.70, found [M+H]⁺: 1197.5.

### Example 86: Compound 86

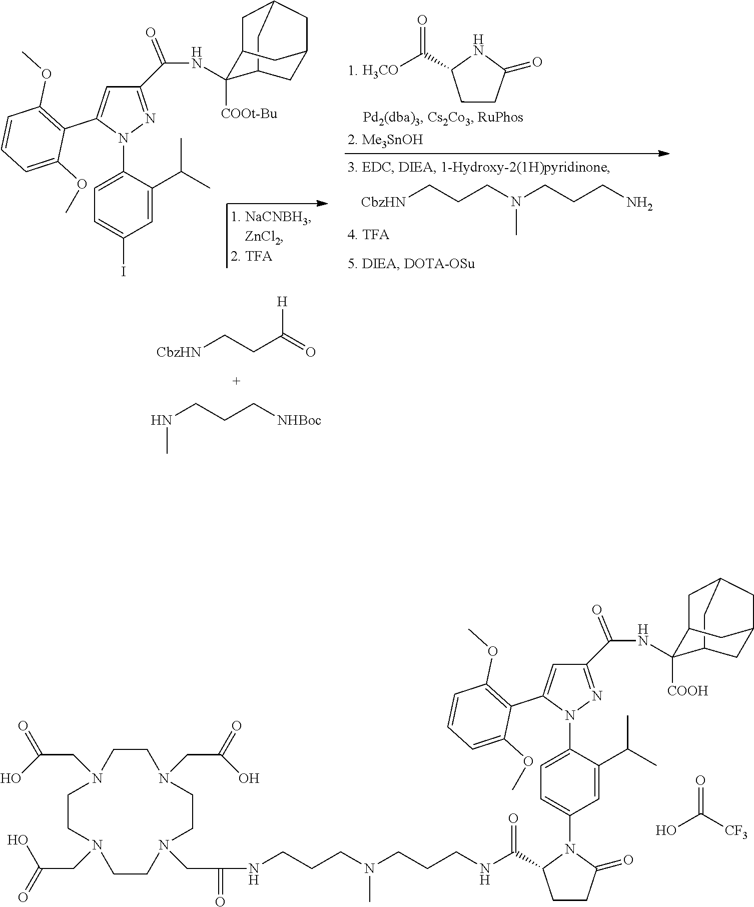

Synthesis of benzyl (3-((3-aminopropyl)(methyl)amino)propyl)carbaminate

Step 1: A mixture of benzyl (3-oxopropyl)carbamate (500 mg, 1 Eq, 2.41 mmol), tert-butyl (3-(methylamino)propyl)carbamate (500 mg, 1.10 Eq, 2.66 mmol), zinc chloride (329 mg, 1.00 Eq, 2.41 mmol) and MeOH (6 mL) was stirred at 20° C. for 30 minutes, then NaBH₃CN (197 mg, 1.30 Eq, 3.13 mmol) was added and the reaction mixture was stirred at 25° C. for 1 hour. The mixture was directly purified by MPLC to afford tert-butyl (3-((3-(((benzyloxy)carbonyl)amino)propyl)(methyl)amino)propyl)carbamate(300 mg, 791 μmol, 32.8%) as a yellow oil. [M+H]⁺=380.2.

Step 2: To a mixture of tert-butyl (3-((3-(((benzyloxy)carbonyl)amino)propyl)(methyl)amino)propyl)carbamate (100 mg, 1 Eq, 264 μmol) and DCM (1.5 mL), was added TFA (0.3 mL) and the reaction mixture was stirred at 25° C. for 1 hour. The mixture was concentrated under reduced pressure to afford benzyl (3-((3-aminopropyl)(methyl)amino)propyl)carbamate (60 mg, 0.21 mmol, 82%) which was used directly in the next step without any purification.

### Synthesis of Compound 86

Step 1: A mixture of tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (400 mg, 1 Eq, 551 μmol), Pd₂(dba)₃ (50 mg, 0.099 Eq, 55 μmol), methyl (R)-5-oxopyrrolidine-2-carboxylate (158 mg, 2.00 Eq, 1.10 mmol), RuPhos (51 mg, 0.20 Eq, 0.11 mmol), Cs₂CO₃ (539 mg, 3.00 Eq, 1.65 mmol) and 1,4-dioxane (6 mL) was stirred at 100° C. for 3 h under N₂. The mixture was directly purified by MPLC to afford methyl (R)-1-(4-(3-(((1R,3S,5R,7R)-2-(tert-butoxycarbonyl)adamantan-2-yl)carbamoyl)-5-(2,6-dimethoxyphenyl)-1-pyrazol-1-yl)-3-isopropylphenyl)-5-oxopyrrolidine-2-carboxylate (105 mg, 142 μmol, 25.7%) as a yellow oil. [M+H]⁺=741.4.

Step 2: A mixture of (R)-1-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)-5-oxopyrrolidine-2-carboxylic acid (75 mg, 1 Eq, 0.10 mmol), trimethylstannanol (93 mg, 3.6 Eq, 0.51 mmol) and DCE (2 mL) was stirred at 90° C. for 16 h. The mixture was concentrated under reduced pressure and the crude product was purified by Prep-HPLC to afford (R)-1-(4-(3-(((1R,3 S,5R,7R)-2-(tert-butoxycarbonyl)adamantan-2-yl)carbamoyl)-5-(2,6-dimethoxyphenyl)-1H-pyrazol-1-yl)-3-isopropylphenyl)-5-oxopyrrolidine-2-carboxylic acid (75 mg, 0.10 mmol, 73%) as a white solid. [M+H]⁺=727.4.

Step 3: A mixture of (R)-1-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)-5-oxopyrrolidine-2-carboxylic acid (75 mg, 1 Eq, 0.10 mmol), EDC (26 mg, 1.3 Eq, 0.14 mmol), 1-Hydroxy-2(1H)pyridinone (15 mg, 1.3 Eq, 0.14 mmol), DIEA (40 mg, 54 μL, 3.0 Eq, 0.31 mmol) and DMF (2 mL) was stirred at 20° C. for 10 minutes, then benzyl (3-((3-aminopropyl)(methyl)amino)propyl)carbamate (37 mg, 1.3 Eq, 0.13 mmol) was added and the reaction mixture was stirred at 25° C. for 3 h. The mixture was directly purified by MPLC to provide tert-butyl (1R,3S,5R,7R)-2-(1-(4-((R)-2-((3-((3-(((benzyloxy)carbonyl)amino)propyl)(methyl)amino)propyl)carbamoyl)-5-oxopyrrolidin-1-yl)-2-isopropylphenyl)-5-(2,6-dimethoxyphenyl)-1H-pyrazole-3-carboxamido)adamantane-2-carboxylate (65 mg, 66 μmol, 64%) as a yellow oil. [M+H]⁺=988.6.

Steps 4-5: tert-Butyl (1R,3S,5R,7R)-2-(1-(4-((R)-2-((3-((3-(((benzyloxy)carbonyl)amino)propyl)(methyl)amino)propyl)carbamoyl)-5-oxopyrrolidin-1-yl)-2-isopropylphenyl)-5-(2,6-dimethoxyphenyl)-1H-pyrazole-3-carboxamido)adamantane-2-carboxylate (65 mg, 1 Eq, 66 μmol) was treated with TFA, then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid, in a manner similar to Example 13, Steps 4 and 5 to provide the 2,2,2-trifluoroacetic acid salt of 2,2′,2″-(10-(2-((3-((3-((R)-1-(4-(3-(((R,3S,5R,7R)-2-carboxyadamantan-2-yl)carbamoyl)-5-(2,6-dimethoxyphenyl)-1H-pyrazol-1-yl)-3-isopropylphenyl)-5-oxopyrrolidine-2-carboxamido)propyl)(methyl)amino)propyl)amino)-2-oxoethyl)-1,4,7,10-tetraazacyclododecane-1,4,7-triyl)triacetic acid (39.6 mg, 30.5 μmol, 53%) as a white solid. MS: Calc'd for C₆₂H₈₆F₃N₁₁O₁₆: 1183.62, found [M+H]⁺: 1184.5.

### Example 87: Compound 87

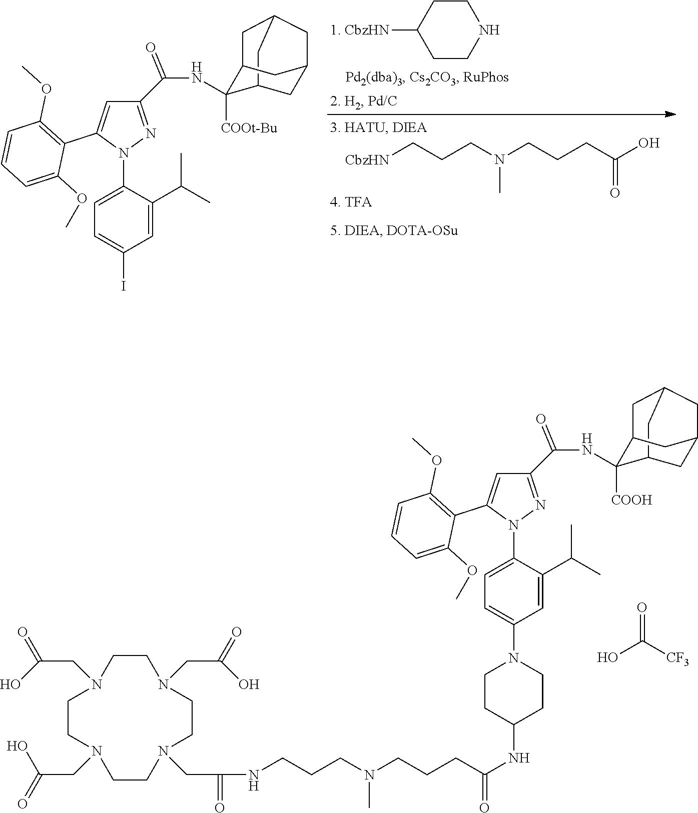

Step 1: A mixture of tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (410 mg, 1 Eq, 565 μmol), RuPhos (52.7 mg, 0.200 Eq, 113 μmol), benzyl piperidin-4-ylcarbamate (331 mg, 2.50 Eq, 1.41 mmol), Pd₂(dba)₃ (51.7 mg, 0.0999 Eq, 56.5 mol), Cs₂CO₃ (552 mg, 3.00 Eq, 1.69 mmol) and 1,4-dioxane(15 mL)under N₂ was stirred at 100° C. for 3 h. The crude product was purified by MPLC to afford tert-butyl (1R,3S,5r,7r)-2-(1-(4-(4-(((benzyloxy)carbonyl)amino)piperidin-1-yl)-2-isopropylphenyl)-5-(2,6-dimethoxyphenyl)-1H-pyrazole-3-carboxamido)adamantane-2-carboxylate (410 mg, 493 μmol, 87.2%) as an off-white solid. [M+H]⁺=832.5.

Step 2: tert-Butyl (1R,3 S,5r,7r)-2-(1-(4-(4-(((benzyloxy)carbonyl)amino)piperidin-1-yl)-2-isopropylphenyl)-5-(2,6-dimethoxyphenyl)-1H-pyrazole-3-carboxamido)adamantane-2-carboxylate was treated with H₂ and Pd/C in a manner similar to Example 1, Step 7 to provide tert-butyl (1R,3 S,5r,7r)-2-(1-(4-(4-aminopiperidin-1-yl)-2-isopropylphenyl)-5-(2,6-dimethoxyphenyl)-1H-pyrazole-3-carboxamido)adamantane-2-carboxylate as white solid.

Steps 3-5: tert-Butyl (1R,3S,5r,7r)-2-(1-(4-(4-aminopiperidin-1-yl)-2-isopropylphenyl)-5-(2,6-dimethoxyphenyl)-1H-pyrazole-3-carboxamido)adamantane-2-carboxylate was treated with 4-((3-(((benzyloxy)carbonyl)amino)propyl)(methyl)amino)butanoic acid, HATU, and DIEA in a manner similar to Example 14, Step 2, then treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″(10-(2-((3-((4-((1-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-4-yl)amino)-4-oxobutyl)(methyl)amino)propyl)amino)-2-oxoethyl)-1,4,7,10-tetraazacyclododecane-1,4,7-triyl)triacetic acid (47.7 mg, 36.7 μmol, 49%) as a white solid. MS: Calc'd for C₆₃H₉₀F₃N₁₁O₁₅: 1183.66, found [M+H]⁺: 1184.6.

### Example 88: Compound 88

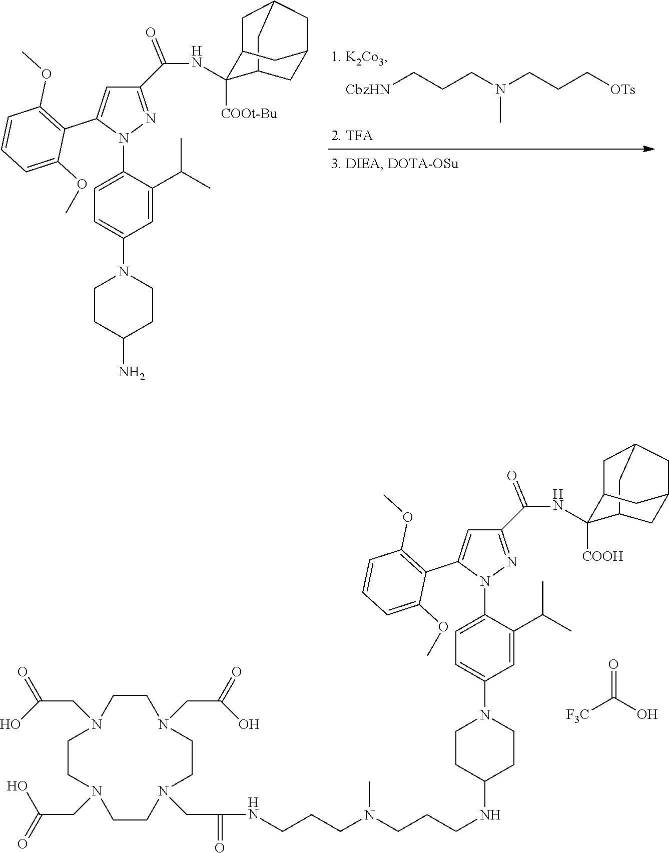

Step 1: A mixture of tert-butyl (1R,3 S,5r,7r)-2-(1-(4-(4-aminopiperidin-1-yl)-2-isopropylphenyl)-5-(2,6-dimethoxyphenyl)-1H-pyrazole-3-carboxamido)adamantane-2-carboxylate (from Example 87, Step 2) (70 mg, 1 Eq, 0.10 mmol), 3-((3-(((benzyloxy)carbonyl)amino)propyl)(methyl)amino)propyl 4-methylbenzenesulfonate (0.13 g, 3.0 Eq, 0.30 mmol), K₂CO₃ (42 mg, 3.0 Eq, 0.30 mmol) and DMF (1.2 mL) was stirred at 80° C. for 2 h. The mixture was directly purified by MPLC to afford tert-butyl (1R,3S,5r,7r)-2-(1-(4-(4-((3-((3-(((benzyloxy)carbonyl)amino)propyl)(methyl)amino)propyl)amino)piperidin-1-yl)-2-isopropylphenyl)-5-(2,6-dimethoxyphenyl)-1H-pyrazole-3-carboxamido)adamantane-2-carboxylate (35 mg, 36 μmol, 36%) as an off-white solid. [M+H]⁺=960.7.

Steps 2-3: tert-Butyl (1R,3S,5r,7r)-2-(1-(4-(4-((3-((3-(((benzyloxy)carbonyl)amino)propyl)(methyl)amino)propyl)amino)piperidin-1-yl)-2-isopropylphenyl)-5-(2,6-dimethoxyphenyl)-1H-pyrazole-3-carboxamido)adamantane-2-carboxylate was treated with TFA, then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid, in a manner similar to Example 13, Steps 4 and 5 to provide the 2,2,2-trifluoroacetic acid salt of 2,2′,2″-(10-(2-((3-((3-(1-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-4-yl)amino)propyl)(methyl)amino)propyl)amino)-2-oxoethyl)-1,4,7,10-tetraazacyclododecane-1,4,7-triyl)triacetic acid (16.1 mg, 12.7 μmol, 33%) as a white solid. MS: Calc'd for C₆₂H₉₀F₃N₁₁O₁₄: 1156.44, found [M+H]⁺: 1156.6.

### Example 89: Compound 89

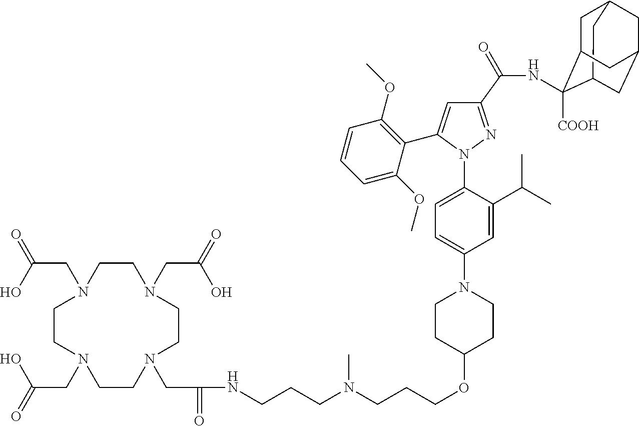 [SMILES: COc1cccc(OC)c1-c1cc(C(=O)NC2(C(=O)O)C3CC4CC(C3)CC2C4)nn1-c1ccc(N2CCC(OCCCN(C)CCCNC(=O)CN3CCN(CC(=O)O)CCN(CC(=O)O)CCN(CC(=O)O)CC3)CC2)cc1C(C)C]

### Example 90: Compound 90

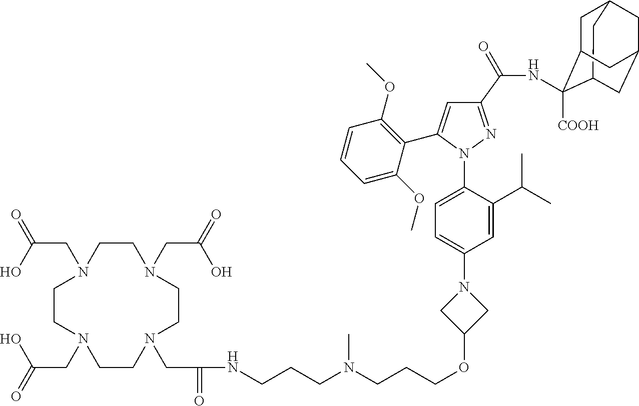 [SMILES: COc1cccc(OC)c1-c1cc(C(=O)NC2(C(=O)O)C3CC4CC(C3)CC2C4)nn1-c1ccc(N2CC(OCCCN(C)CCCNC(=O)CN3CCN(CC(=O)O)CCN(CC(=O)O)CCN(CC(=O)O)CC3)C2)cc1C(C)C]

### Example 91: Compound 91

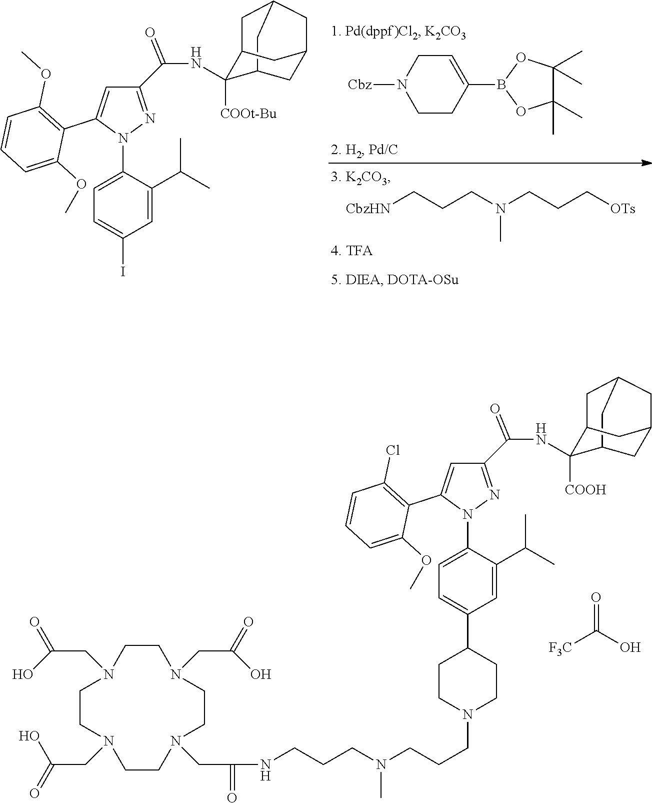

Step 1: A mixture of tert-butyl (1R,3S,5r,7r)-2-(5-(2-chloro-6-methoxyphenyl)-1-(4-iodo-2-isopropylphenyl)-1H-pyrazole-3-carboxamido)adamantane-2-carboxylate (1.0 g, 1 Eq, 1.4 mmol), 1,1′-bis(diphenylphosphino)ferrocene-palladium(II) dicholoride (0.10 g, 0.10 Eq, 0.14 mmol), benzyl 4-(4,4,5,5-tetramethyl-1,3,2-dioxaborolan-2-yl)-3,6-dihydropyridine-1(2H₄)-carboxylate (0.94 g, 2.0 Eq, 2.7 mmol), K₂CO₃ (0.57 g, 3.0 Eq, 4.1 mmol), 1,4-dioxane (10 mL) and H₂O (1 mL) under N2 was stirred at 70° C. for 2 h. The mixture was directly purified by M/PLC to afford benzyl 4-(4-(3-(((1R,3S,5r,7r)-2-(tert-butoxycarbonyl)adamantan-2-yl)carbamoyl)-5-(2-chloro-6-methoxyphenyl)-1H-pyrazol-1-yl)-3-isopropylphenyl)-3,6-dihydropyridine-1(2H)-carboxylate (800 mg, 976 μmol, 71%) as a yellow solid.

Step 2. Benzyl 4-(4-(3-(((1R,3 S,5r,7r)-2-(tert-butoxycarbonyl)adamantan-2-yl)carbamoyl)-5-(2-chloro-6-methoxyphenyl)-1H-pyrazol-1-yl)-3-isopropylphenyl)-3,6-dihydropyridine-1(2H)-carboxylate was treated with H₂ and Pd/C in a manner similar to Example 1, Step 7 to provide tert-butyl (1R,3S,5r,7r)-2-(5-(2-chloro-6-methoxyphenyl)-1-(2-isopropyl-4-(piperidin-4-yl)phenyl)-1H-pyrazole-3-carboxamido)adamantane-2-carboxylate (230 mg, 335 mol, 38.1%) as a white solid. [M+H]⁺=687.6.

Steps 3-5: tert-Butyl (1R,3S,5r,7r)-2-(5-(2-chloro-6-methoxyphenyl)-1-(2-isopropyl-4-(piperidin-4-yl)phenyl)-1H-pyrazole-3-carboxamido)adamantane-2-carboxylate was treated with 3-((3-(((benzyloxy)carbonyl)amino)propyl)(methyl)amino)propyl 4-methylbenzenesulfonate in manner similar to Example 88, Step 1 then was treated with TFA, then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid, in a manner similar to Example 13, Steps 4 and 5 to afford the 2,2,2-trifluoroacetic acid salt of 2,2′,2″-(10-(2-((3-((3-(4-(4-(3-(((1R,3S,5r,7r)-2-carboxyadamantan-2-yl)carbamoyl)-5-(2-chloro-6-methoxyphenyl)-1H-pyrazol-1-yl)-3-isopropylphenyl)piperidin-1-yl)propyl)(methyl)amino)propyl)amino)-2-oxoethyl)-1,4,7,10-tetraazacyclododecane-1,4,7-triyl)triacetic acid (1.6 mg, 1.3 μmol, 4.8%) as a white solid. MS: Calc'd for C₅₉H₈₆ClN₁₀O₁₁: 1144.61, found [M+H]⁺: 1145.5.

### Example 92: Compound 92

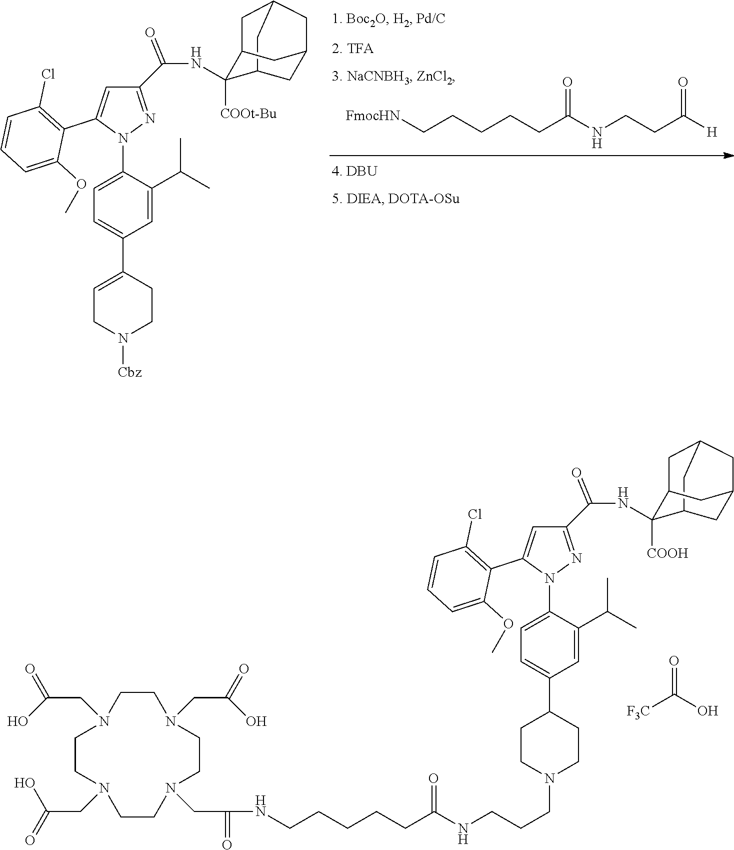

Step 1: To a mixture of benzyl 4-(4-(5-(2-chloro-6-methoxyphenyl)-3-(((1R,3S,5r,7r)-2-(pivaloyloxy)adamantan-2-yl)carbamoyl)-1H-pyrazol-1-yl)-3-isopropylphenyl)-3,6-dihydropyridine-1(2H)-carboxylate (60 mg, 1 Eq, 73 μmol) and MeOH (4 mL) under N₂ was carefully added Boc₂O (32 mg, 34 μL, 2.0 Eq, 0.15 mmol) then Pd/C (23 mg, 10% Wt, 0.30 Eq, 22 μmol). The flask was evacuated and flushed with hydrogen three times then the reaction mixture was stirred for 25° C. at 1 hour. The reaction mixture was filtered through a pad of celite, the filtrate was concentrated and purified by MPLC to provide tert-butyl 4-(4-(5-(2-chloro-6-methoxyphenyl)-3-(((1R,3S,5r,7r)-2-(pivaloyloxy)adamantan-2-yl)carbamoyl)-1H-pyrazol-1-yl)-3-isopropylphenyl)piperidine-1-carboxylate (30 mg, 38 μmol, 52%) as a white solid.

Step 2: tert-Butyl 4-(4-(5-(2-chloro-6-methoxyphenyl)-3-(((1R,3S,5r,7r)-2-(pivaloyloxy)adamantan-2-yl)carbamoyl)-1H-pyrazol-1-yl)-3-isopropylphenyl)piperidine-1-carboxylate was treated with TFA in a manner similar to Example 1, Step 8 to provide (1R,3S,5r,7r)-2-(5-(2-chloro-6-methoxyphenyl)-1-(2-isopropyl-4-(piperidin-4-yl)phenyl)-1H-pyrazole-3-carboxamido)adamantane-2-carboxylic acid (20 mg, 32 μmol, 83%) as a white oil. [M+H]⁺=631.3.

Step 3: A mixture of (1R,3S,5r,7r)-2-(5-(2-chloro-6-methoxyphenyl)-1-(2-isopropyl-4-(piperidin-4-yl)phenyl)-1H-pyrazole-3-carboxamido)adamantane-2-carboxylic acid (20 mg, 1 Eq, 32 μmol), (9H-fluoren-9-yl)methyl (6-oxo-6-((3-oxopropyl)amino)hexyl)carbamate (26 mg, 2.0 Eq, 64 μmol), ZnCl₂ (13 mg, 6.0 μL, 3.0 Eq, 95 μmol) and MeOH (2 mL) was stirred at 25° C. for 10 minutes, then sodium cyanoborohydride (4.0 mg, 3.7 μL, 2.0 Eq, 64 μmol) was added and the reaction mixture was stirred at 25° C. for an additional 1 hour. The mixture was directly purified by MPLC to afford (1R,3S,5r,7r)-2-(1-(4-(1-(3-(6-((((9H-fluoren-9-yl)methoxy)carbonyl)amino)hexanamido)propyl)piperidin-4-yl)-2-isopropylphenyl)-5-(2-chloro-6-methoxyphenyl)-1H-pyrazole-3-carboxamido)adamantane-2-carboxylic acid (15 mg, 15 μmol, 46%) as a white oil. [ML+H]⁺=1023.6.

Steps 4-5: (1R,3S,5r,7r)-2-(1-(4-(1-(3-(6-((((9H-fluoren-9-yl)methoxy)carbonyl)amino)hexanamido)propyl)piperidin-4-yl)-2-isopropylphenyl)-5-(2-chloro-6-methoxyphenyl)-1H-pyrazole-3-carboxamido)adamantane-2-carboxylic acid was treated with DBU in a manner similar to Example 68, Step 5 then 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 13, Steps 5 to provide the trifluoroacetic salt of 2,2′,2′-(10-(2-((6-((3-(4-(4-(3-(((1R,3 S,5r,7r)-2-carboxyadamantan-2-yl)carbamoyl)-5-(2-chloro-6-methoxyphenyl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid (4.8 mg, 3.7 μmol, 30%) as a white solid. MS: Calc'd for C₆₁H₈₇ClN₁₀O₁₂: 1186.62, found [M+H]⁺: 1187.5.

### Example 93: Compound 93

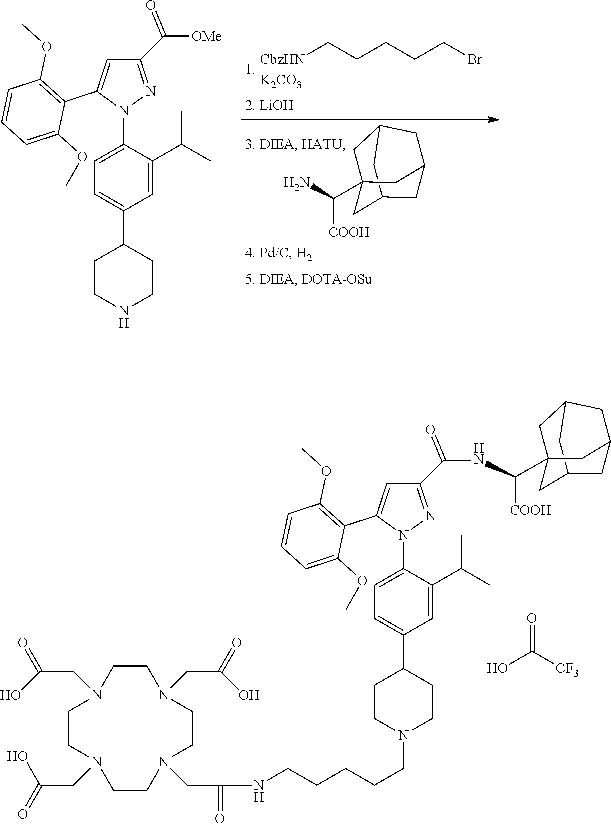

Step 1: A mixture of methyl 5-(2,6-dimethoxyphenyl)-1-(2-isopropyl-4-(piperidin-4-yl)phenyl)-1H-pyrazole-3-carboxylate (160 mg, 1 Eq, 345 μmol), benzyl (5-bromopentyl)carbamate (155 mg, 1.50 Eq, 516 μmol), K₂CO₃ (143 mg, 3.00 Eq, 1.03 mmol) and MeCN (4 mL). The reaction mixture was stirred at 80° C. for 2 h. The mixture was directly purified by MPLC to afford methyl 1-(4-(1-(5-(((benzyloxy)carbonyl)amino)pentyl)piperidin-4-yl)-2-isopropylphenyl)-5-(2,6-dimethoxyphenyl)-11H-pyrazole-3-carboxylate (100 mg, 146 μmol, 42.4%) as a yellow oil. [M+H]⁺=683.8.

Step 2: A mixture of methyl 1-(4-(1-(5-(((benzyloxy)carbonyl)amino)pentyl)piperidin-4-yl)-2-isopropylphenyl)-5-(2,6-dimethoxyphenyl)-1H-pyrazole-3-carboxylate (100 mg, 1 Eq, 146 μmol), LiOH (12 mg, 3.4 Eq, 0.50 mmol), MeOH (3 mL) and H₂O (1 mL) was stirred at 40° C. for 1 hour. The reaction mixture was concentrated under reduced pressure to remove most of the MeOH, the residue was diluted with water (30 mL), the pH value was adjusted to 6.0 by addition of a saturated aqueous solution of NaHSO₄, extracted with DCM (40 mL×3), dried over anhydrous Na₂SO₄, then concentrated under reduced pressure to afford 1-(4-(1-(5-(((benzyloxy)carbonyl)amino)pentyl)piperidin-4-yl)-2-isopropylphenyl)-5-(2,6-dimethoxyphenyl)-11H-pyrazole-3-carboxylic acid (70 mg, 0.10 mmol, 71%) as a light yellow solid, which was used directly in the next step without any purification. [M+H]⁺=669.8.

Step 3: To a mixture of 1-(4-(1-(5-(((benzyloxy)carbonyl)amino)pentyl)piperidin-4-yl)-2-isopropylphenyl)-5-(2,6-dimethoxyphenyl)-1H-pyrazole-3-carboxylic acid (70 mg, 1 Eq, 0.10 mmol), (2S)-2-((3R,5R)-adamantan-1-yl)-2-aminoacetic acid (30 mg, 1.4 Eq, 0.14 mmol) and DMF (2 mL), was added HATU (44 mg, 1.1 Eq, 0.12 mmol) and DIEA (41 mg, 55 μL, 3.0 Eq, 0.32 mmol). The reaction mixture was stirred at 25° C. for 1 hour. The mixture was directly purified by Prep-HPLC to afford (2S)-2-((3R,5R)-adamantan-1-yl)-2-(1-(4-(1-(5-(((benzyloxy)carbonyl)amino)pentyl)piperidin-4-yl)-2-isopropylphenyl)-5-(2,6-dimethoxyphenyl)-1H-pyrazole-3-carboxamido)acetic acid (60 mg, 70 mol, 67%) as a white solid.

(2S)-2-((3R,5R)-adamantan-1-yl)-2-(1-(4-(1-(5-(((benzyloxy)carbonyl)amino)pentyl)piperidin-4-yl)-2-isopropylphenyl)-5-(2,6-dimethoxyphenyl)-1H-pyrazole-3-carboxamido)acetic acid was treated with hydrogen and Pd/C in a manner similar to Example 1, Step 7 then treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 13, Step 5 to provide the 2,2,2-trifluoroacetic acid salt of 2,2′2″-(10-(2-((5-(4-(4-(3-(((1S)-((3R,5R)-adamantan-1-yl)(carboxy)methyl)carbamoyl)-5-(2,6-dimethoxyphenyl)-1H-pyrazol-1-yl)-3-isopropylphenyl)piperidin-1-yl)pentyl)amino)-2-oxoethyl)-1,4,7,10-tetraazacyclododecane-1,4,7-triyl)triacetic acid (22.4 mg, 18.3 μmol, 30%) as a white solid. MS: Calc'd for C₅₉H₈₅N₉O₁₂: 1111.63, found [M+H]⁺: 1112.5.

### Example 94: Compound 94

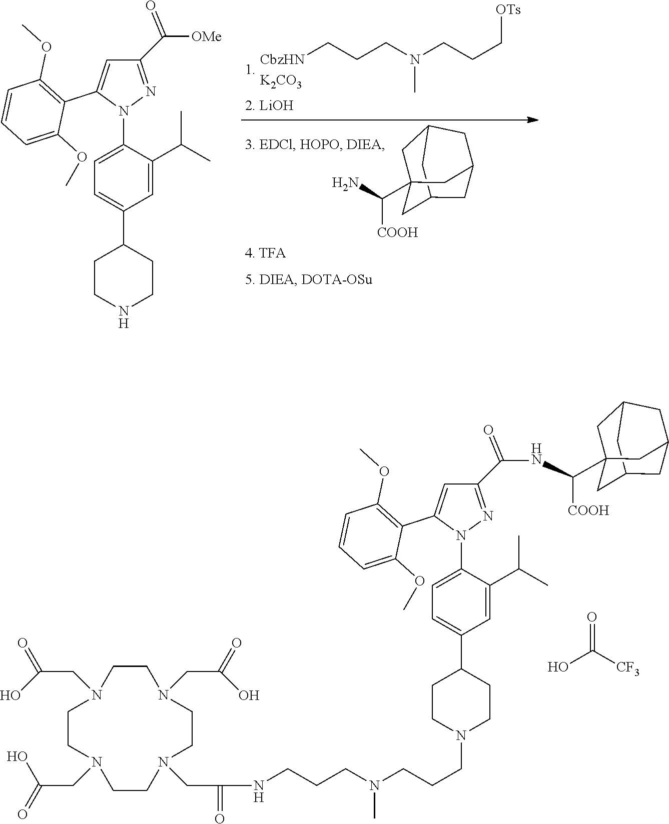

Steps 1-2: Methyl 5-(2,6-dimethoxyphenyl)-1-(2-isopropyl-4-(piperidin-4-yl)phenyl)-1H-pyrazole-3-carboxylate was treated with K₂CO₃ and 3-((3-(((benzyloxy)carbonyl)amino)propyl)(methyl)amino)propyl 4-methylbenzenesulfonate in a manner similar to in a manner similar to Example 13, Step 3 then treated with LiOH in a manner similar to Example 1, Step 4 to provide 1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxylic acid (130 mg, 183 μmol, 88.4%) as a white solid. [M+H]⁺=712.5.

Step 3: A mixture of 1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxylic acid (130 mg, 1 Eq, 183 μmol), EDCl (35 mg, 1.0 Eq, 0.18 mmol), HOPO (20 mg, 0.99 Eq, 0.18 mmol), DIEA (71 mg, 96 μL, 3.0 Eq, 0.55 mmol) and DMF (2 mL) under N₂ was stirred at 25° C. for 5 h. The mixture was directly purified by MPLC to afford (2S)-2-((3R,5R)-adamantan-1-yl)-2-(1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxamido)acetic acid (45 mg, 50 μmol, 27%) as a yellow solid. [M+H]⁺=903.6.

Step 4: A mixture of (2S)-2-((3R,5R)-adamantan-1-yl)-2-(1-(4-(1-(3-((3-(((benzyloxy)carbonyl)amino)propyl)(methyl)amino)propyl)piperidin-4-yl)-2-isopropylphenyl)-5-(2,6-dimethoxyphenyl)-1H-pyrazole-3-carboxamido)acetic acid (45 mg, 1 Eq, 50 μmmol) and TFA (2 mL) was stirred at 70° C. for 1 hour. The mixture was concentrated under reduced pressure to afford (2S)-2-((3R,5R)-adamantan-1-yl)-2-(1-(4-(1-(3-((3-aminopropyl)(methyl)amino)propyl)piperidin-4-yl)-2-isopropylphenyl)-5-(2,6-dimethoxyphenyl)-1H-pyrazole-3-carboxamido)acetic acid (35 mg, 46 μmol, 91%) as a yellow oil. [M+H]⁺=769.6.

Step 5: (2S)-2-((3R,5R)-adamantan-1-yl)-2-(1-(4-(1-(3-((3-aminopropyl)(methyl)amino)propyl)piperidin-4-yl)-2-isopropylphenyl)-5-(2,6-dimethoxyphenyl)-1H-pyrazole-3-carboxamido)acetic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 13, Step 5 to provide the 2,2,2-trifluoroacetic acid salt of 2,2′,2″-(10-(2-((3-((3-(4-(4-(3-(((1S)-((3R,5R)-adamantan-1-yl)(carboxy)methyl)carbamoyl)-5-(2,6-dimethoxyphenyl)-1H-pyrazol-1-yl)-3-isopropylphenyl)piperidin-1-yl)propyl)(methyl)amino)propyl)amino)-2-oxoethyl)-1,4,7,10-tetraazacyclododecane-1,4,7-triyl)triacetic acid (30.0 mg, 23.6 μmol, 52%) as a white solid. MS: Calc'd for C₆₁H₉₀N₁₀O₁₂: 1154.67, found [M+H]⁺: 1155.9.

### Example 95: Compound 95

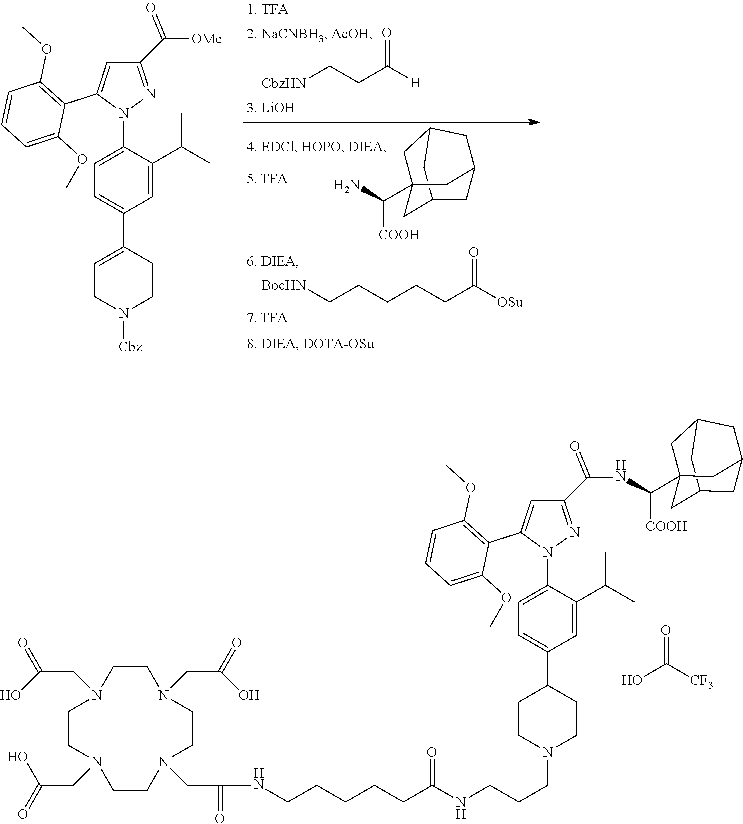

Step 1: A mixture of benzyl 4-(4-(5-(2,6-dimethoxyphenyl)-3-(methoxycarbonyl)-1H-pyrazol-1-yl)-3-isopropylphenyl)-3,6-dihydropyridine-1(2H)-carboxylate (600 mg, 1 Eq, 1.01 mmol) and TFA (6 mL) was stirred at 60° C. for 1 hour. The collected fractions were dried by lyophilization to afford methyl 5-(2,6-dimethoxyphenyl)-1-(2-isopropyl-4-(1,2,3,6-tetrahydropyridin-4-yl)phenyl)-1H-pyrazole-3-carboxylate (600 mg, 983 μmol, 97.6%) as a white solid. [M+H]⁺=4629.

Step 2: To a mixture of methyl 5-(2,6-dimethoxyphenyl)-1-(2-isopropyl-4-(1,2,3,6-tetrahydropyridin-4-yl)phenyl)-1H-pyrazole-3-carboxylate (600 mg, 1 Eq, 1.30 mmol) and MeOH (6 mL) was added benzyl (3-oxopropyl)carbamate (323 mg, 1.2 Eq, 1.56 mmol), NaBH₃CN (123 mg, 1.5 Eq, 1.95 mmol) and AcOH (390 mg, 372 μL, 5 Eq, 6.50 mmol). The reaction mixture was stirred at 25° C. for 4 h. The mixture was directly purified by Prep-HPLC to provide methyl 1-(4-(1-(3-(((benzyloxy)carbonyl)amino)propyl)-1,2,3,6-tetrahydropyridin-4-yl)-2-isopropylphenyl)-5-(2,6-dimethoxyphenyl)-1H-pyrazole-3-carboxylate (350 mg, 531 μmol, 40.8%) as a white solid. [M+H]⁺=653.3.

Steps 3-5: Methyl 1-(4-(1-(3-(((benzyloxy)carbonyl)amino)propyl)-1,2,3,6-tetrahydropyridin-4-yl)-2-isopropylphenyl)-5-(2,6-dimethoxyphenyl)-1H-pyrazole-3-carboxylate was treated with LiOH, then (2S)-2-((3R,5R)-adamantan-1-yl)-2-aminoacetic acid, then TFA in manner similar to Example 94, Steps 2-4 to provide (2S)-2-((3R,5R)-adamantan-1-yl)-2-(1-(4-(1-(3-aminopropyl)piperidin-4-yl)-2-isopropylphenyl)-5-(2,6-dimethoxyphenyl)-1H-pyrazole-3-carboxamido)acetic acid (40 mg, 53 μmol, 110%) as a white solid. [M+H]⁺=698.6.

Step 6: To a mixture of (2S)-2-((3R,5R)-adamantan-1-yl)-2-(1-(4-(1-(3-aminopropyl)piperidin-4-yl)-2-isopropylphenyl)-5-(2,6-dimethoxyphenyl)-1H-pyrazole-3-carboxamido)acetic acid (40 mg, 1 Eq, 57 μmol), DIEA (7.4 mg, 10 μL, 1 Eq, 57 μmol) and DMF (1 mL) was added 2,5-dioxopyrrolidin-1-yl 6-((tert-butoxycarbonyl)amino)hexanoate (19 mg, 1 Eq, 57 μmol) and the reaction mixture was stirred at 25° C. for 1 h. The mixture was directly purified by Prep-HPLC to afford (2S)-2-((3R,5R)-adamantan-1-yl)-2-(1-(4-(1-(3-(6-((tert-butoxycarbonyl)amino)hexanamido)propyl)piperidin-4-yl)-2-isopropylphenyl)-5-(2,6-dimethoxyphenyl)-1H-pyrazole-3-carboxamido)acetic acid (20 mg, 19 μmol, 33%) as a white solid. [M+H]⁺=911.8.

Steps 7-8: (2S)-2-((3R,5R)-Adamantan-1-yl)-2-(1-(4-(1-(3-(6-((tert-butoxycarbonyl)amino)hexanamido)propyl)piperidin-4-yl)-2-isopropylphenyl)-5-(2,6-dimethoxyphenyl)-1H-pyrazole-3-carboxamido)acetic acid was treated with TFA then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid, in a manner similar to Example 13, Steps 4 and 5 to afford the 2,2,2-trifluoroacetic acid salt of 2,2′,2″-(10-(2-((6-((3-(4-(4-(3-(((1 S)-((3R,5R)-adamantan-1-yl)(carboxy)methyl)carbamoyl)-5-(2,6-dimethoxyphenyl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid (3.6 mg, 2.6 mol, 14%) as a white solid. MS: Calc'd for C₆₃H₉₂N₁₀O₁₃: 1196.68, found [M+H]⁺: 1197.6.

### Example 96: Compound 96

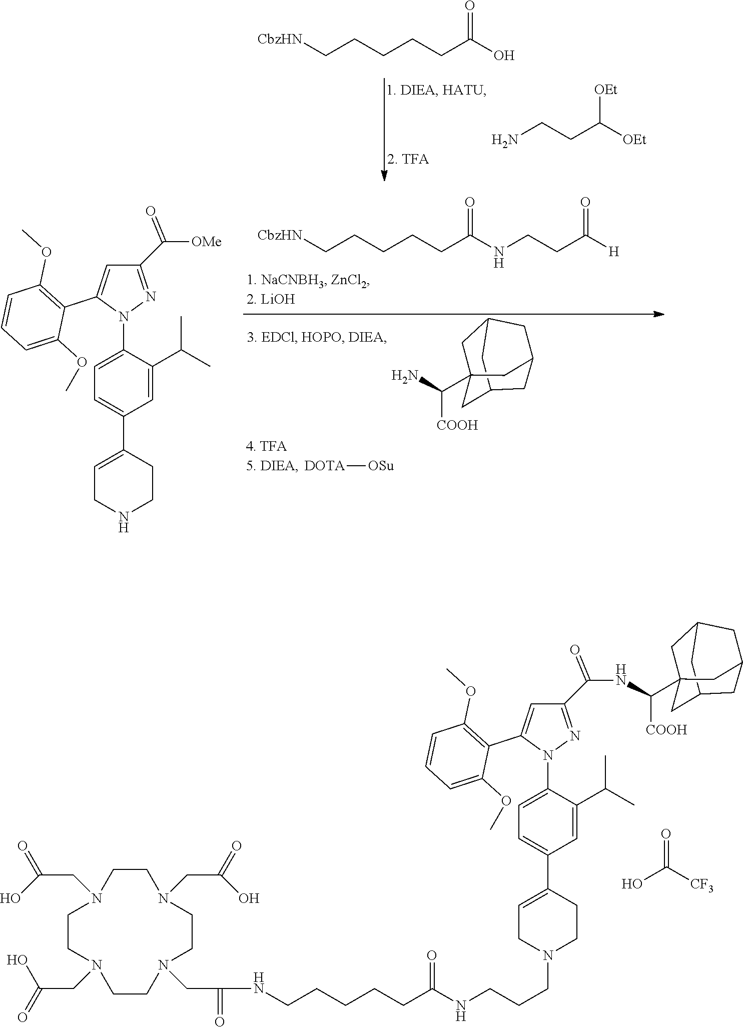

Step 1: To a mixture of 6-(((benzyloxy)carbonyl)amino)hexanoic acid (1.0 g, 1 Eq, 3.8 mmol), DEA (1.5 g, 2.0 mL, 3.1 Eq, 12 mmol) and DMF (10 mL) was added 3,3-diethoxypropan-1-amine (0.67 g, 1.2 Eq, 4.6 mmol) and HATU (2.9 g, 2.0 Eq, 7.6 mmol). The reaction mixture was stirred at 25° C. for 1 hour. The mixture was directly purified by Prep-HPLC to afford benzyl (6-((3,3-diethoxypropyl)amino)-6-oxohexyl)carbamate (1.5 g, 3.1 mmol, 84%) as a white solid. [M-44]⁺=349.3.

Step 2: A mixture of benzyl (6-((3,3-diethoxypropyl)amino)-6-oxohexyl)carbamate (500 mg, 1 Eq, 1.27 mmol), TFA (0.2 mL), and H₂O (2 mL) was stirred at 25° C. for 2 h to afford benzyl (6-oxo-6-((3-oxopropyl)amino)hexyl)carbamate (410 mg, 1.28 mmol, 101%) as a white solid. [M+H]⁺=727.4.

Step 3: To a mixture of methyl 5-(2,6-dimethoxyphenyl)-1-(2-isopropyl-4-(1,2,3,6-tetrahydropyridin-4-yl)phenyl)-1H-pyrazole-3-carboxylate (130 mg, 1 Eq, 282 μmol) and DCE (2 mL) was added benzyl (6-oxo-6-((3-oxopropyl)amino)hexyl)carbamate (180 mg, 1.99 Eq, 562 μmol), sodium cyanoborohydride (53.1 mg, 49.2 μL, 3.00 Eq, 845 μmol) and zinc chloride (76.8 mg, 35.5 μL, 2.00 Eq, 564 μmol). The reaction mixture was stirred at 25° C. for 1 h. The mixture was directly purified by Prep-HPLC to afford methyl 1-(4-(1-(3-(6-(((benzyloxy)carbonyl)amino)hexanamido)propyl)-1,2,3,6-tetrahydropyridin-4-yl)-2-isopropylphenyl)-5-(2,6-dimethoxyphenyl)-1H-pyrazole-3-carboxylate (250 mg, 326 μmol, 116%) as a white solid.

Steps 4-7: The trifluoroacetic acid salt of 2,2′,2″-(10-(2-((6-((3-(4-(4-(3-(((1 S)-((3R,5R)-Adamantan-1-yl)(carboxy)methyl)carbamoyl)-5-(2,6-dimethoxyphenyl)-1H-pyrazol-1-yl)-3-isopropylphenyl)-3,6-dihydropyridin-1(2H)-yl)propyl)amino)-6-oxohexyl)amino)-2-oxoethyl)-1,4,7,10-tetraazacyclododecane-1,4,7-triyl)triacetic acid was synthesized from methyl 1-(4-(1-(3-(6-(((benzyloxy)carbonyl)amino)hexanamido)propyl)-1,2,3,6-tetrahydropyridin-4-yl)-2-isopropylphenyl)-5-(2,6-dimethoxyphenyl)-1H-pyrazole-3-carboxylate in a manner similar to Steps 3-5 and 8 of Example 95 to provide the product (27 mg, 19 μmol, 27%) as a white solid. MS: Calc'd for C₆₃H₉₀N₁₀O₁₃: 1194.67, found [M+H]⁺: 1195.5.

### Example 97: Compound 97

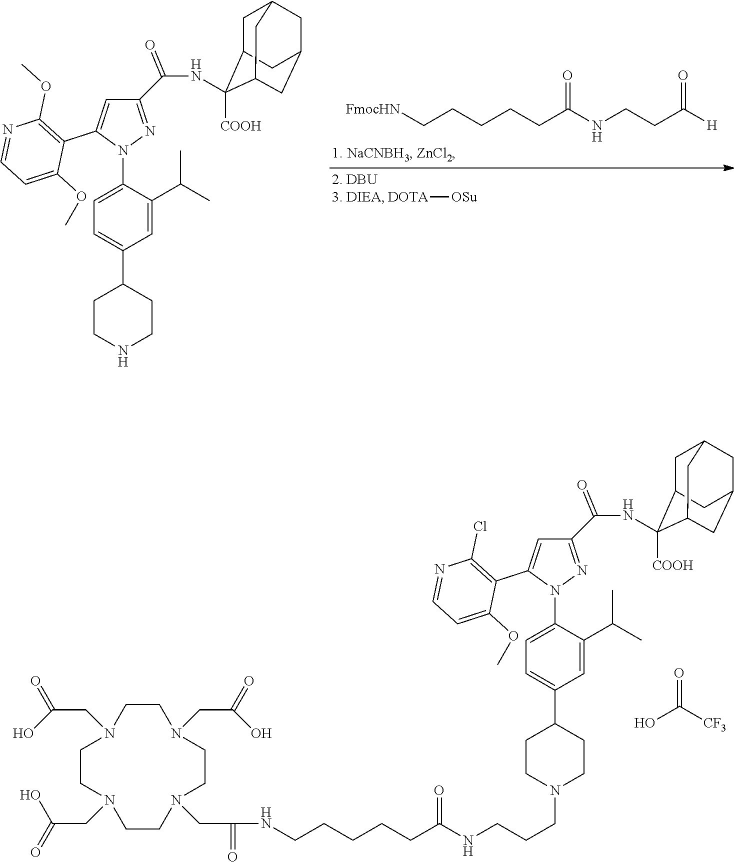

Steps 1-3: 2,2′,2″-(10-(2-((6-((3-(4-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid was synthesized from (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylic acid (Compound 83) and (9HH-fluoren-9-yl)methyl (6-oxo-6-((3-oxopropyl)amino)hexyl)carbamate in a manner similar to Step 3 of Example 96, Step 5 of Example 68, then Step 5 of Example 13 to provide the 2,2,2-trifluoroacetic acid salt of the product (18.4 mg, 14.2 mol, 57%) as a white solid. MS: Calc'd for C₆₁H₈₉N₁₁O₁₃: 1183.66, found [M+H]⁺: 1184.6.

### Example 98: Compound 98

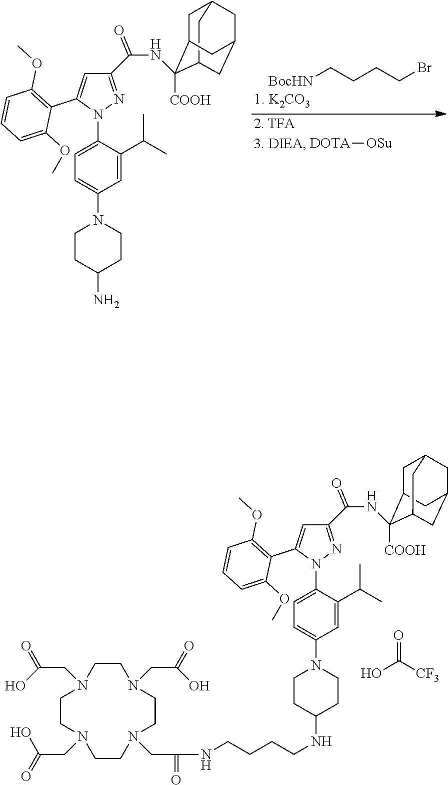

Step 1: A mixture of tert-butyl (1R,3S,5r,7r)-2-(1-(4-aminopiperidin-1-yl-2-isopropylphenyl)-5-(2,6-dimethoxyphenyl)-1H-pyrazole-3-carboxamido)adamantane-2-carboxylate (60 mg, 1 Eq, 86 μmol), K₂CO₃ (36 mg, 3.0 Eq, 0.26 mmol), tert-butyl (4-bromobutyl)carbamate (43 mg, 2.0 Eq, 0.17 mmol) and DMF (1.5 mL) was stirred at 80° C. for 2 h. The mixture was directly purified by MPLC to afford tert-butyl (1R,3S,5r,7r)-2-(1-(4-(4-((4-((tert-butoxycarbonyl)amino)butyl)amino)piperidin-1-yl)-2-isopropylphenyl)-5-(2,6-dimethoxyphenyl)-1H-pyrazole-3-carboxamido)adamantane-2-carboxylate (50 mg, 58 mol, 67%) as an off-yellow solid. [M+H]⁺=869.6.

Steps 2-3: tert-Butyl (1R,3S,5r,7r)-2-(1-(4-(4-((4-((tert-butoxycarbonyl)amino)butyl)amino)piperidin-1-yl)-2-isopropylphenyl)-5-(2,6-dimethoxyphenyl)-1H-pyrazole-3-carboxamido)adamantane-2-carboxylate was treated with TFA then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to afford the 2,2,2-trifluoroacetic acid salt of 2,2′,2″-(10-(2-((4-((l-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-4-yl)amino)butyl)amino)-2-oxoethyl)-1,4,7,10-tetraazacyclododecane-1,4,7-triyl)triacetic acid (32.2 mg, 26.5 μmol, 38%) as a white solid. MS: Calc'd for C₅₇H₈₂N₁₀O₁₂: 1098.61, found [M+H]⁺: 1099.7.

### Example 99: Compound 99

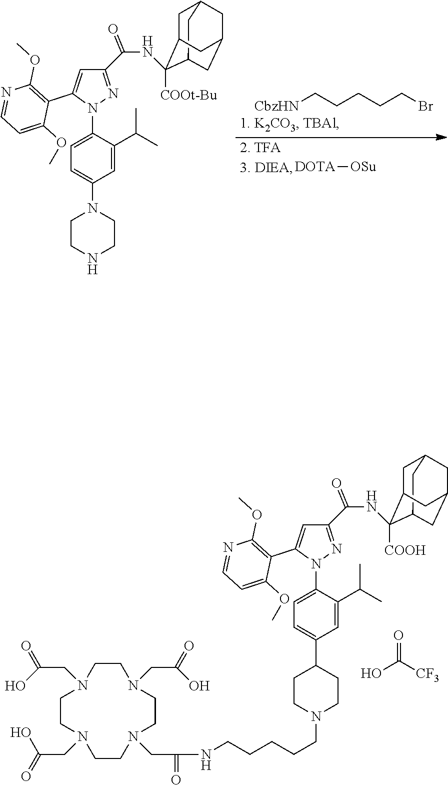

Step 1: A mixture of tert-butyl (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylate (50 tug, 1 Eq, 73 μmol), K₂CO₃ (30 μg, 3.0 Eq, 0.22 mmol), TBAI (54 mg, 2.0 Eq, 0.15 mmol) and DMF (1.5 mL) was stirred at 20° C. for 10 minutes, then benzyl (5-bromopentyl)carbamate (44 mg, 2.0 Eq, 0.15 mmol) was added and the reaction mixture was stirred at 60° C. for an additional 1 hour. The mixture was directly purified by MPLC to afford tert-butyl (1R,3 S,5r,7r)-2-(1-(4-(1-(5-(((benzyloxy)carbonyl)amino)pentyl)piperidin-4-yl)-2-isopropylphenyl)-5-(2,4-dimethoxypyridin-3-yl)-1H-pyrazole-3-carboxamido)adamantane-2-carboxylate (35 mg, 39 μmol, 53%) as a yellow oil.

Step 2: tert-Butyl (1R,3 S,5r,7r)-2-(1-(4-(1-(5-(((benzyloxy)carbonyl)amino)pentyl)piperidin-4-yl)-2-isopropylphenyl)-5-(2,4-dimethoxypyridin-3-yl)-1H-pyrazole-3-carboxamido)adamantane-2-carboxylate was treated with TFA then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to afford the 2,2,2-trifluoroacetic acid salt of 2,2′,2″-(10-(2-((5-(4-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)piperidin-1-yl)pentyl)amino)-2-oxoethyl)-1,4,7,10-tetraazacyclododecane-1,4,7-triyl)triacetic acid (13.1 mg, 10.8 μmol, 31%) as a white solid. MS: Calc'd for C₅₇H₈₂N₁₀O₁₂: 1098.61, found [M+H]⁺: 1099.5.

### Synthesis of Compound 99-Lu

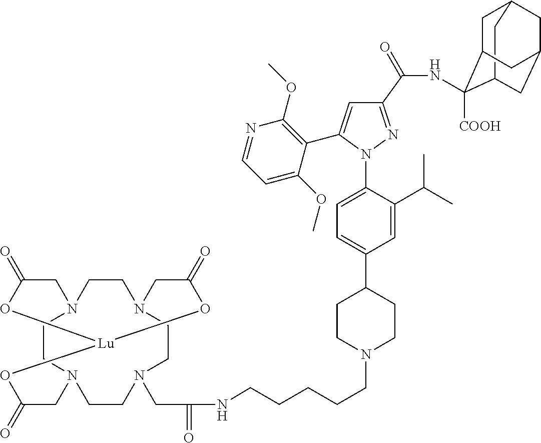

A mixture of 2,2′,2″-(10-(2-((5-(4-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)piperidin-1-yl)pentyl)amino)-2-oxoethyl)-1,4,7,10-tetraazacyclododecane-1,4,7-triyl)triacetic acid (25 mg, 1 Eq, 23 μmol), lutetium(III) chloride (19 mg, 3.0 Eq, 68 μmol) and NaOAc buffer (2 mL) was stirred at 80° C. for 30 min. The crude mixture was directly purified by Prep-HPLC to provide the product (22.8 mg, 16.4 μmol, 72%) as a white solid. MS: Calc'd for C₅₇H₇₉LuN₁₀O₁₂: 1270.53, found [M+H]⁺: 1271.6.

### Example 100: Compound 100

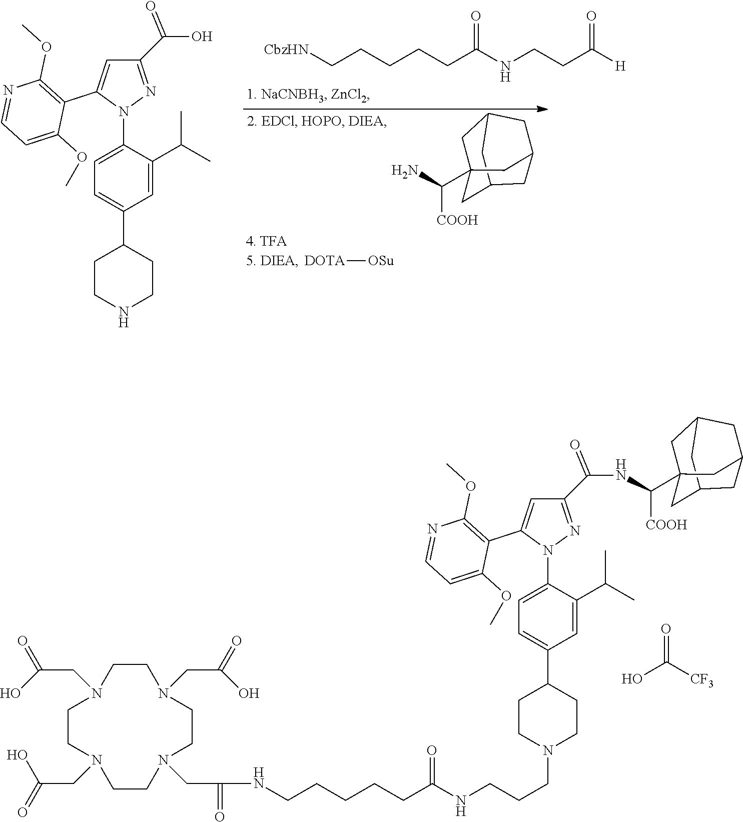

Step 1: 1-(4-(1-(3-(6-(((Benzyloxy)carbonyl)amino)hexanamido)propyl)piperidin-4-yl)-2-isopropylphenyl)-5-(2,4-dimethoxypyridin-3-yl)-1H-pyrazole-3-carboxylic acid was synthesized from 5-(2,4-dimethoxypyridin-3-yl)-1-(2-isopropyl-4-(piperidin-4-yl)phenyl)-1H-pyrazole-3-carboxylic acid (150 μg, 1 Eq, 333 mol) and benzyl (6-oxo-6-((3-oxopropyl)amino)hexyl)carbamate (128 mg, 1.20 Eq, 400 μmol) in a manner similar to Example 97, Step 1 to provide the product (120 mg, 159 μmol, 47.7%) as a white solid. [M+H]⁺=755.5.

Step 2: 1-(4-(1-(3-(6-(((Benzyloxy)carbonyl)amino)hexanamido)propyl)piperidin-4-yl)-2-isopropylphenyl)-5-(2,4-dimethoxypyridin-3-yl)-1H-pyrazole-3-carboxylic acid was treated with (2S)-2-((3R,5R)-adamantan-1-yl)-2-aminoacetic acid in a manner similar to Example 94, Step 3 to provide (2S)-2-((3R,5R)-adamantan-1-yl)-2-(1-(4-(1-(3-(6-(((benzyloxy)carbonyl)amino)hexanamido)propyl)piperidin-4-yl)-2-isopropylphenyl)-5-(2,4-dimethoxypyridin-3-yl)-1H-pyrazole-3-carboxamido)acetic acid (40 mg, 42 μmol, 27%) as a white solid. [M+H]⁺=946.5.

Steps 3-4: (2S)-2-((3R,5R)-adamantan-1-yl)-2-(1-(4-(1-(3-(6-(((benzyloxy)carbonyl)amino)hexanamido)propyl)piperidin-4-yl)-2-isopropylphenyl)-5-(2,4-dimethoxypyridin-3-yl)-1H-pyrazole-3-carboxamido)acetic acid was treated with TFA then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to afford the 2,2,2-trifluoroacetic acid salt of 2,2′,2″-(10-(2-((6-((3-(4-(4-(3-(((1S)-((3R,5R)-adamantan-1-yl)(carboxy)methyl)carbamoyl)-5-(2,4-dimethoxypyridin-3-yl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid (23 mg, 19 μmol, 49%) as a white solid. MS: Calc'd for C₆₂H₉₁N₁₁O₁₃: 1197.68, found [M+H]⁺: 1198.6.

### Example 101: Compound 101

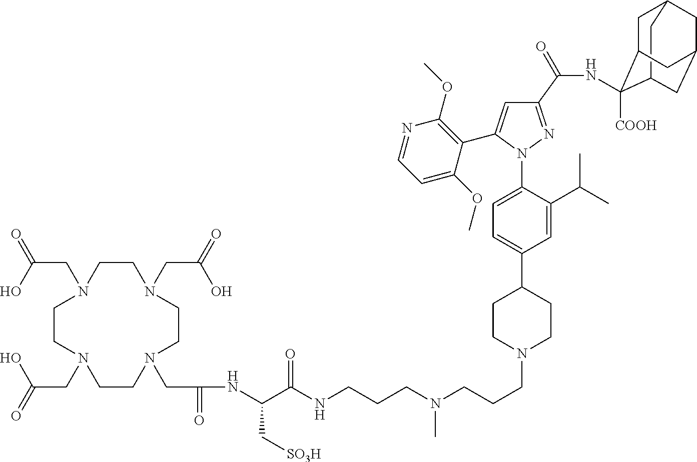 [SMILES: COc1ccnc(OC)c1-c1cc(C(=O)NC2(C(=O)O)C3CC4CC(C3)CC2C4)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C]

Steps 1-2: (1R,3S,5r,7r)-2-(1-(4-(1-(3-((3-Aminopropyl)(methy)amino)propyl)piperidin-4-yl)-2-isopropylphenyl)-5-(2,4-dimethoxypyridin-3-yl)-1H-pyrazole-3-carboxamido)adamantane-2-carboxylic acid was synthesized from tert-butyl (1R,3 S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylate and 3-((3-(((benzyloxy)carbonyl)amino)propyl)(methyl)amino)propyl 4-methylbenzenesulfonate in a manner similar to Example 88, Steps 1-2 to afford the product (50 mg, 0.060 mmol, 94%,) which was directly used for next step without further purification. [M+H]⁺=756.5.

Step 3: A mixture of (((9H-fluoren-9-yl)methoxy)carbonyl)(sulfo)-D-alanine (47 mg, 1.5 Eq, 0.12 mmol), HATU (36 mg, 1.2 Eq, 95 μmol), DIEA (10 mg, 14 μL, 1 Eq, 79 μmol) and DMF (1.5 mL) was stirred at 20° C. for 10 minutes, then (1R,3S,5r,7r)-2-(1-(4-(1-(3-((3-aminopropyl)(methyl)amino)propyl)piperidin-4-yl)-2-isopropylphenyl)-5-(2,4-dimethoxypyridin-3-yl)-1H-pyrazole-3-carboxamido)adamantane-2-carboxylic acid (60 mg, 1 Eq, 79 μmol) was added and the reaction mixture was stirred at 25° C. for an additional 1 hour. The mixture was directly purified by MPLC to afford (2R)-2-((((9H-fluoren-9-yl)methoxy)carbonyl)amino)-3-((3-((3-(4-(4-(3-(((1R,3S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)piperidin-1-yl)propyl)(methyl)amino)propyl)amino)-3-oxopropane-1-sulfonic acid (35 mg, 30 μmol, 37%) as a yellow oil.

Step 4: (2R)-2-((((9H-fluoren-9-yl)methoxy)carbonyl)amino)-3-((3-((3-(4-(4-(3-(((1R,3 S,5r,7r)-2-(tert-butoxycarbonyl)adamantan-2-yl)carbamoyl)-5-(2,4-dimethoxypyridin-3-yl)-11H-pyrazol-1-yl)-3-isopropylphenyl)piperidin-1-yl)propyl)(methyl)amino)propyl)amino)-3-oxopropane-1-sulfonic acid was treated with TFA then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to afford the 2,2,2-trifluoroacetic acid salt of 2,2′,2″-(10-(2-(((2R)-1-((3-((3-(4-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)piperidin-1-yl)propyl)(methyl)amino)propyl)amino)-1-oxo-3-sulfopropan-2-yl)amino)-2-oxoethyl)-1,4,7,10-tetraazacyclododecane-1,4,7-triyl)triacetic acid (6.9 mg, 4.9 μmol, 30%) as a white solid. MS: Calc'd for C₆₂H₉₂N₁₂O₁₆S: 1292.65, found [M+H]⁺: 1293.9.

### Example 102: Compound 102

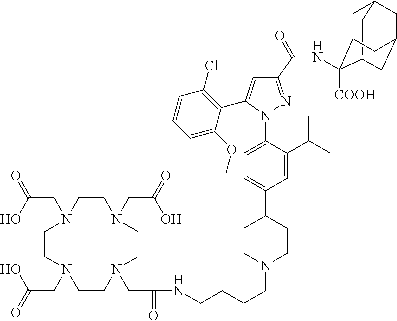 [SMILES: COc1cccc(Cl)c1-c1cc(C(=O)NC2(C(=O)O)C3CC4CC(C3)CC2C4)nn1-c1ccc(C2CCN(CCCCNC(=O)CN3CCN(CC(=O)O)CCN(CC(=O)O)CCN(CC(=O)O)CC3)CC2)cc1C(C)C]

Steps 1-3: 2,2,2″-(10-(2-((4-(4-(4-(3-(((1R,3S,5r,7r)-2-Carboxyadamantan-2-yl)carbamoyl)-5-(2-chloro-6-methoxyphenyl)-1H-pyrazol-1-yl)-3-isopropylphenyl)piperidin-1-yl)butyl)amino)-2-oxoethyl)-1,4,7,10-tetraazacyclododecane-1,4,7-triyl)triacetic acid was synthesized from tert-butyl (1R,3S,5r,7r)-2-(5-(2-chloro-6-methoxyphenyl)-1-(2-isopropyl-4-(piperidin-4-yl)phenyl)-1H-pyrazole-3-carboxamido)adamantane-2-carboxylate (160 μg, 1 Eq, 233 μmol) and tert-butyl (4-bromobutyl)carbamate in a manner similar to Example 85, Step 1, then treated with TFA then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to afford the 2,2,2-trifluoroacetic acid salt of the product (24.7 mg, 20.5 mol, 29%) as a white solid. MS: Calc'd for C₅₆H₇₈ClN₉O₁₁: 1087.55, found [M+H]⁺: 1088.4.

### Example 103: Compound 103

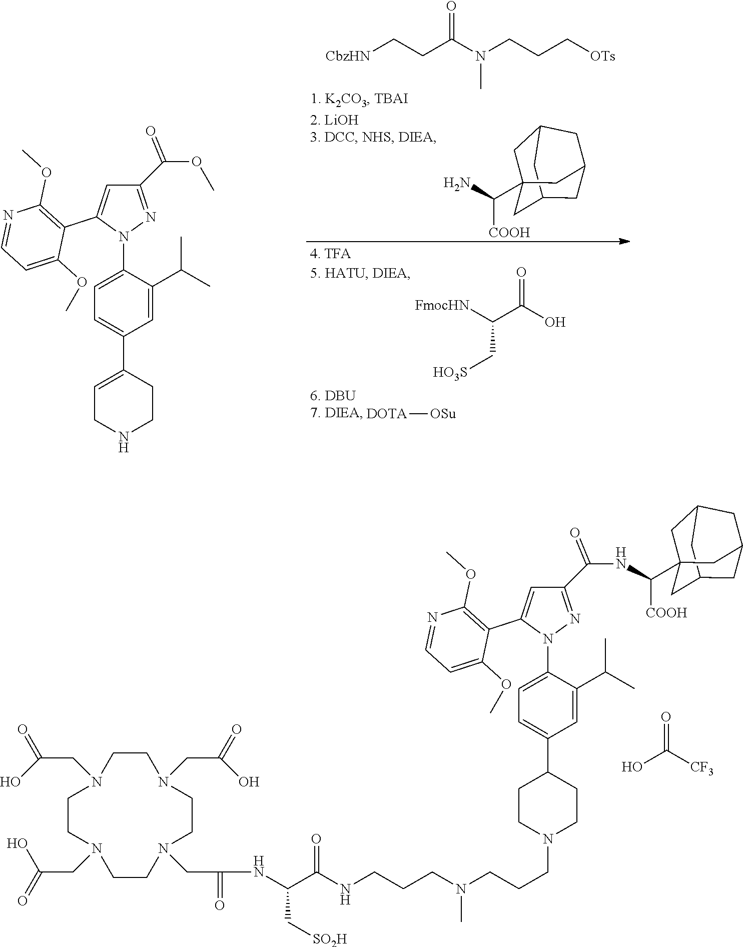

Step 1: A mixture of (((91H-fluoren-9-yl)methoxy)carbonyl)(sulfo)-D-alanine (41 mg, 2.0 Eq, 0.10 mmol), HATU (30 mg, 1.5 Eq, 79 μmol), DIEA (20 mg, 27 μL, 3.0 Eq, 0.15 mmol) and DMF (1 mL) was stirred at 20° C. for 10 minutes, then (2S)-2-((3R,5R)-adamantan-1-yl)-2-(1-(4-(1-(3-((3-aminopropyl)(methyl)amino)propyl)piperidin-4-yl)-2-isopropylphenyl)-5-(2,4-dimethoxypyridin-3-yl)-1H-pyrazole-3-carboxamido)acetic acid (40 mg, 1 Eq, 52 μmol) was added and the reaction mixture was stirred at 25° C. for an additional 1 h. The mixture was directly purified by MPLC to afford (2S)-2-(1-(4-(1-((R)-1-(9H-fluoren-9-yl)-11-methyl-3,6-dioxo-5-(sulfomethyl)-2-oxa-4,7,11-triazatetradecan-14-yl)piperidin-4-yl)-2-isopropylphenyl)-5-(2,4-dimethoxypyridin-3-yl)-1H-pyrazole-3-carboxamido)-2-((3R,5R)-adamantan-1-yl)acetic acid (20 mg, 17 μmol, 34%) as a yellow oil. [M+H]⁺=1021.6.

Step 2: (2S)-2-(1-(4-(1-((R)-1-(9H-Fluoren-9-yl)-11-methyl-3,6-dioxo-5-(sulfomethyl)-2-oxa-4,7,11-triazatetradecan-14-yl)piperidin-4-yl)-2-isopropylphenyl)-5-(2,4-dimethoxypyridin-3-yl)-1H-pyrazole-3-carboxamido)-2-((3R,5R)-adamantan-1-yl)acetic acid was treated with LiOH in a manner similar to Example 1, Step 4 to provide 1-(4-(1-(3-((3-(((benzyloxy)carbonyl)amino)propyl)(methyl)amino)propyl)piperidin-4-yl)-2-isopropylphenyl)-5-(2,4-dimethoxypyridin-3-yl)-1H-pyrazole-3-carboxylic acid (75 mg, 0.11 mmol, 96%) as a yellow oil. [M+H]⁺=713.5.

Step 3: To a mixture of 1-(4-(1-(3-((3-(((benzyloxy)carbonyl)amino)propyl)(methyl)amino)propyl)piperidin-4-yl)-2-isopropylphenyl)-5-(2,4-dimethoxypyridin-3-yl)-1H-pyrazole-3-carboxylic acid (70 mg, 1 Eq, 98 μmol), N-hydroxysuccinimide (17 mg, 1.5 Eq, 0.15 mmol) and THF (2 mL), was added DCC (41 mg, 2.0 Eq, 0.20 mmol) under N₂. The reaction mixture was stirred at 25° C. for 1 hour. The mixture was filtered and the filtrate was concentrated to provide a crude intermediate. To a mixture of DIEA (13 mg, 17 μL, 1 Eq, 98 μmol), (2S)-2-((3R,5R)-adamantan-1-yl)-2-aminoacetic acid (41 mg 2.0 Eq, 0.20 mmol), THF (2 mL) and H₂O (0.5 mL) was added a solution of the crude intermediate in THF (2 mL) dropwise, then the reaction mixture was stirred at 25° C. for an additional 1 hour. The crude mixture was directly purified by Prep-HPLC to afford (2S)-2-((3R,5R)-adamantan-1-yl)-2-(1-(4-(1-(3-((3-(((benzyloxy)carbonyl)amino)propyl)(methyl)amino)propyl)piperidin-4-yl)-2-isopropylphenyl)-5-(2,4-dimethoxypyridin-3-yl)-1H-pyrazole-3-carboxamido)acetic acid (50 mg, 55 μmol, 56%) as a white solid. [M+H]⁺=904.7.

Steps 4-5: (2S)-2-((3R,5R)-adamantan-1-yl)-2-(1-(4-(1-(3-((3-(((benzyloxy)carbonyl)amino)propyl)(methyl)amino)propyl)piperidin-4-yl)-2-isopropylphenyl)-5-(2,4-dimethoxypyridin-3-yl)-1H-pyrazole-3-carboxamido)acetic acid was treated with TFA in a manner similar to Example 1, Step 8 then was treated with (((9H-fluoren-9-yl)methoxy)carbonyl)(sulfo)-D-alanine in a manner similar to Example 101, Step 2 to provide (2S)-2-(1-(4-(1-((R)-1-(9H-fluoren-9-yl)-11-methyl-3,6-dioxo-5-(sulfomethyl)-2-oxa-4,7,11-triazatetradecan-14-yl)piperidin-4-yl)-2-isopropylphenyl)-5-(2,4-dimethoxypyridin-3-yl)-1H-pyrazole-3-carboxamido)-2-((3R,5R)-adamantan-1-yl)acetic acid (20 mg, 17 μmol, 34%) as a yellow oil. [M+H]⁺=1021.6.

Steps 6-7: (2S)-2-(1-(4-(1-((R)-1-(9H-Fluoren-9-yl)-11-methyl-3,6-dioxo-5-(sulfomethyl)-2-oxa-4,7,11-triazatetradecan-14-yl)piperidin-4-yl)-2-isopropylphenyl)-5-(2,4-dimethoxypyridin-3-yl)-1H-pyrazole-3-carboxamido)-2-((3R,5R)-adamantan-1-yl)acetic acid was treated with DBU in a manner similar to Example 68, Step 5 then treated with DIEA and 2,2′,2″(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-(((2R)-1-((3-((3-(4-(4-(3-(((1 S)-((3R,5R)-adamantan-1-yl)(carboxy)methyl)carbamoyl)-5-(2,4-dimethoxypyridin-3-yl)-1H-pyrazol-1-yl)-3-isopropylphenyl)piperidin-1-yl)propyl)(methyl)amino)propyl)amino)-1-oxo-3-sulfopropan-2-yl)amino)-2-oxoethyl)-1,4,7,10-tetraazacyclododecane-1,4,7-triyl)triacetic acid (5.6 mg, 3.9 μmol, 24%) as a white solid. MS: Calc'd for C₆₃H₉₄N₁₂O₁₆S: 1306.66, found [M+H]⁺: 1307.7.

### Example 104: Compound 104

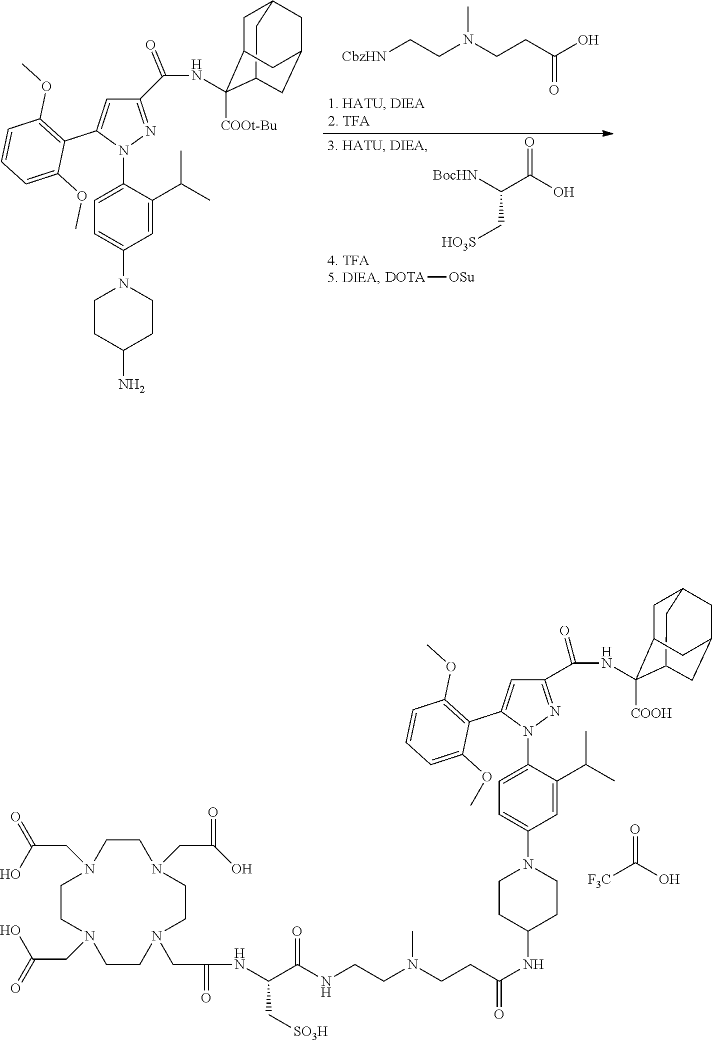

Step 1: A mixture of 3-((2-(((benzyloxy)carbonyl)amino)ethyl)(methyl)amino)propanoic acid (127 mg, 3.01 Eq, 453 μmol), HATU (68.6 mg, 1.20 Eq, 180 μmol), DIEA (58.3 mg, 78.6 μL, 3.00 Eq, 451 μmol) and DMF (5 mL) was stirred at 25° C. for 10 minutes, then tert-butyl (1R,3S,5r,7r)-2-(1-(4-(4-aminopiperidin-1-yl)-2-isopropylphenyl)-5-(2,6-dimethoxyphenyl)-1H-pyrazole-3-carboxamido)adamantane-2-carboxylate (105 mg, 1 Eq, 150 μmol) was added and the reaction mixture was stirred at 25° C. for an additional 1 h. The mixture was directly purified by MPLC to provide (1R,3S,5r,7r)-2-(1-(4-(4-(3-((2-(((benzyloxy)carbonyl)amino)ethyl)(methyl)amino)propanamido)piperidin-1-yl)-2-isopropylphenyl)-5-(2,6-dimethoxyphenyl)-H-pyrazole-3-carboxamido)adamantan-2-yl pivalate (28 mg, 29 μmol, 19%) as a white solid.

Step 2: (1R,3S,5r,7r)-2-(1-(4-(4-(3-((2-(((Benzyloxy)carbonyl)amino)ethyl)(methyl)amino)propanamido)piperidin-1-yl)-2-isopropylphenyl)-5-(2,6-dimethoxyphenyl)-1Tl-pyrazole-3-carboxamido)adamantan-2-yl pivalate was treated with TFA at 60° C. for 1 hour to provide (1R,3S,5r,7r)-2-(1-(4-(4-(3-((2-aminoethyl)(methyl)amino)propanamido)piperidin-1-yl)-2-isopropylphenyl)-5-(2,6-dimethoxyphenyl)-1H-pyrazole-3-carboxamido)adamantane-2-carboxylic acid (30 mg, 27 μmol, 94%) as a white oil.

Steps 3-5: (1R,3S,5r,7r)-2-(1-(4-(4-(3-((2-Aminoethyl)(methyl)amino)propanamido)piperidin-1-yl)-2-isopropylphenyl)-5-(2,6-dimethoxyphenyl)-1H-pyrazole-3-carboxamido)adamantane-2-carboxylic acid was treated with (tert-butoxycarbonyl)(sulfo)-D-alanine in a manner similar to Example 103, Step 5 then treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-(((2S)-1-((2-((3-((1-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-4-yl)amino)-3-oxopropyl)(methyl)amino)ethyl)amino)-1-oxo-3-(sulfinooxy)propan-2-yl)amino)-2-oxoethyl)-1,4,7,10-tetraazacyclododecane-1,4,7-triyl)triacetic acid (6.0 mg, 4.2 mol, 19%) as a white solid. MS: Calc'd for C₆₂H₉₀NO₁₂O₁₇S: 1306.63, found [M+H]⁺: 1307.6.

### Example 105: Compound 105

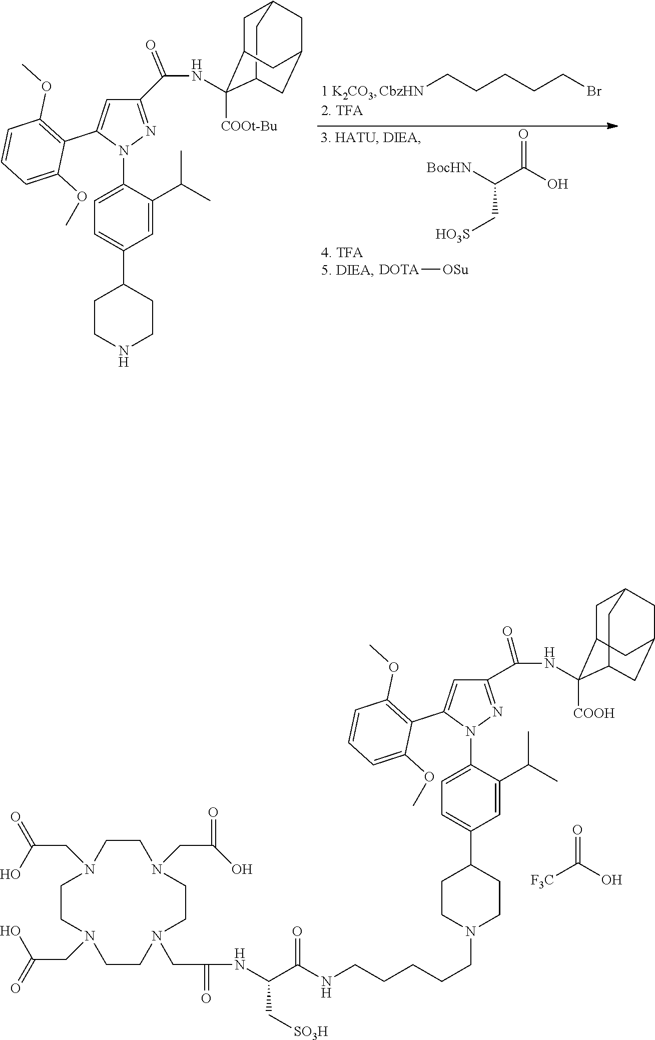

Steps 1-2: tert-Butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (200 μg, 1 Eq, 293 μmol) was treated with benzyl (5-bromopentyl)carbamate (130 mg, 1.48 Eq, 433 μmol) in a manner similar to Example 98, Step 1 then treated with TFA in a manner similar to Example 104, Step 2 to provide (1R,3S,5r,7r)-2-(1-(4-(1-(5-aminopentyl)piperidin-4-yl)-2-isopropylphenyl)-5-(2,6-dimethoxyphenyl)-1H-pyrazole-3-carboxamido)adamantane-2-carboxylic acid (105 mg, 0.12 mmol, 89%) as a white oil. [M+H]⁺=712.5.

Steps 3-5: 2,2′,2′-(10-(2-(((R)-1-((5-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)pentyl)amino)-1-oxo-3-sulfopropan-2-yl)amino)-2-oxoethyl)-1,4,7,10-tetraazacyclododecane-1,4,7-triyl)triacetic acid was synthesized from (1R,3S,5r,7r)-2-(1-(4-(1-(5-aminopentyl)piperidin-4-yl)-2-isopropylphenyl)-5-(2,6-dimethoxyphenyl)-1H-pyrazole-3-carboxamido)adamantane-2-carboxylic acid and (tert-butoxycarbonyl)(sulfo)-D-alanine in a manner similar to Example 104, Steps 3-5 to provide the 2,2,2-trifluoroacetic acid salt of the product as a white solid. MS: Calc'd for C₆₁H₈₈N₁₀O₁₆S: 1248.61, found [M+H]⁺: 1249.6.

### Example 106: Compound 106

Synthesis of N2-((benzyloxy)carbonyl)-N5-(3-(2-nitro-1H-imidazol-1-yl)propyl)-L-glutamine

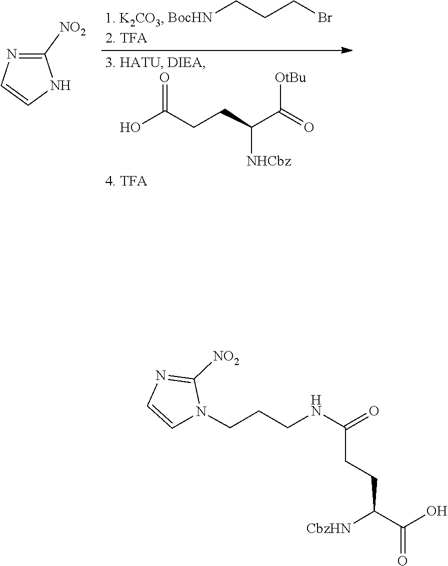 [SMILES: CC(C)(C)OC(=O)NCCCBr ; O=[N+]([O-])c1ncc[nH]1 ; CC(C)(C)OC(=O)[C@H](CCC(=O)O)NC(=O)OCc1ccccc1 ; O=C(CC[C@H](NC(=O)OCc1ccccc1)C(=O)O)NCCCn1ccnc1[N+](=O)[O-]]

N2-((benzyloxy)carbonyl)-N5-(3-(2-nitro-1H-imidazol-1-yl)propyl)-L-glutamine was synthesized from 2-nitro-1H-imidazole (2.0 g, 1 Eq, 18 mmol) and tert-butyl (3-bromopropyl)carbamate (3.8 g, 0.90 Eq, 16 mmol) in a manner similar to that shown in Example 105 to provide the product (450 mg, 0.93 mmol, 91%) as a brown oil.

### Synthesis of Compound 106

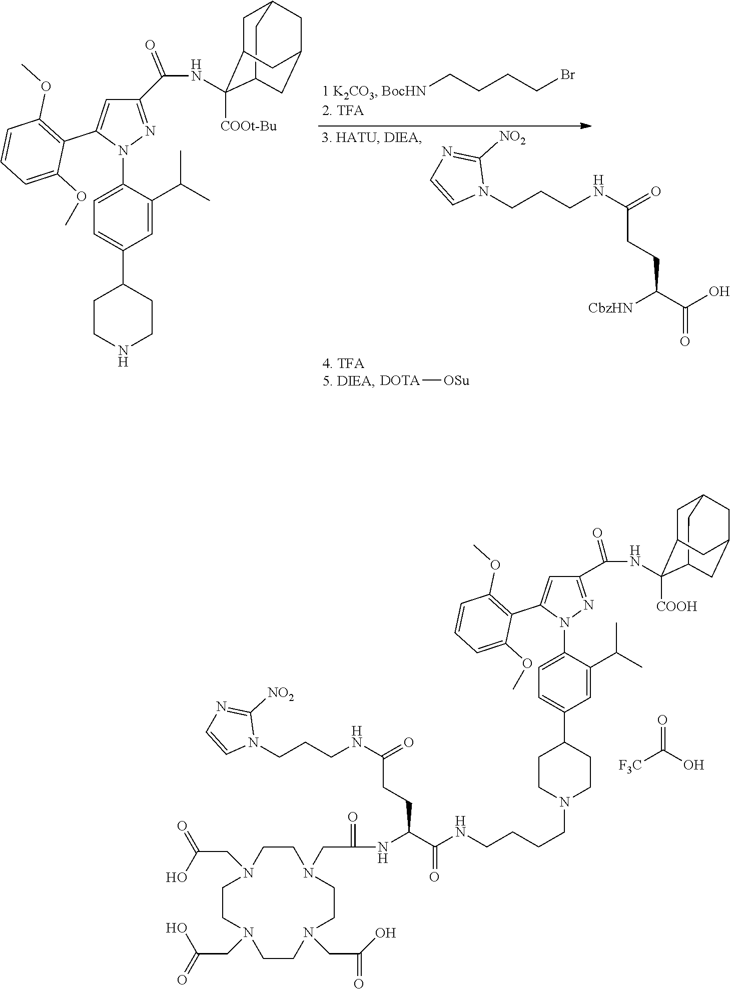

Step 1: 2,2′,2″-(10-(2-(((S)-1-((4-(4-(4-(3-(((1R,3S,5r,7r)-2-Carboxyadamantan-2-yl)carbamoyl)-5-(2,6-dimethoxyphenyl)-1H-pyrazol-1-yl)-3-isopropylphenyl)piperidin-1-yl)butyl)amino)-5-((3-(2-nitro-1H-imidazol-1-yl)propyl)amino)-1,5-dioxopentan-2-yl)amino)-2-oxoethyl)-1,4,7,10-tetraazacyclododecane-1,4,7-triyl)triacetic acid was synthesized from tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate in a manner similar to Example 104 to provide the 2,2,2-trifluoroacetic acid salt of the product (17.2 mg, 10.8 μmol, 42%) as a light yellow solid. MS: Calc'd for C₆₈H₉₆N₁₄O₁₆: 1364.71, found [M+H]⁺: 1365.7.

### Example 107: Compound 107

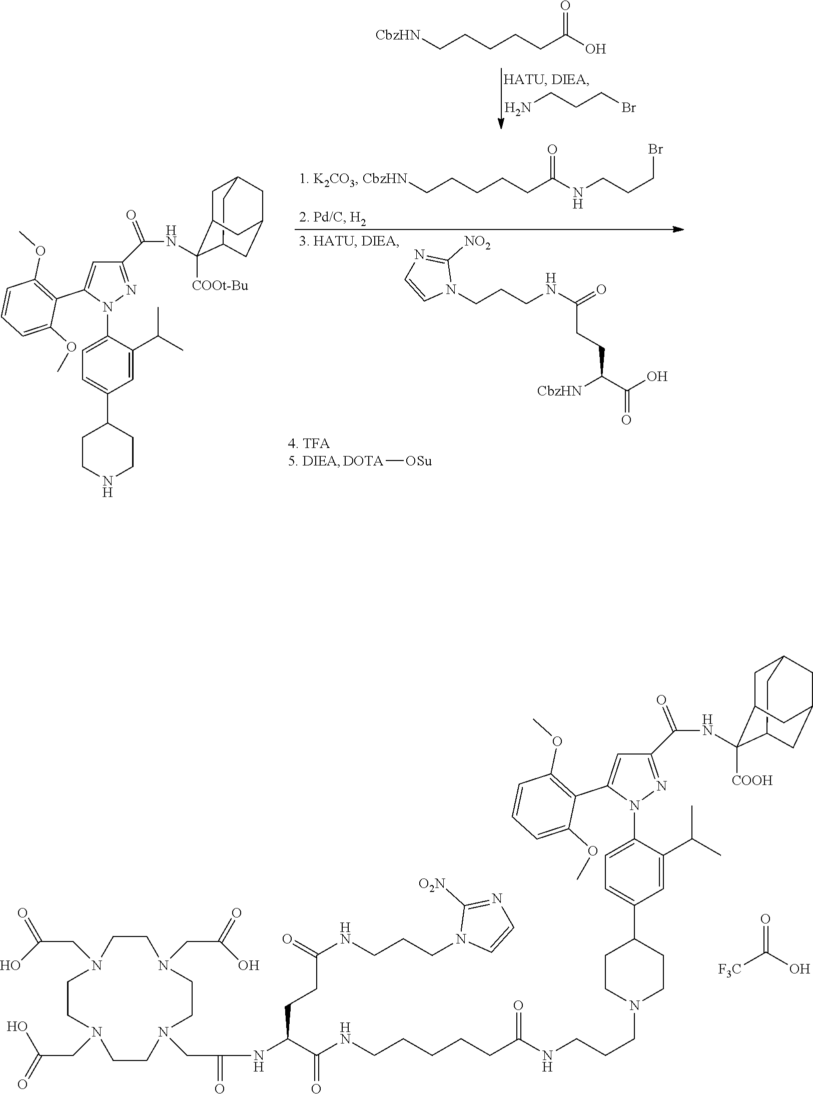

Step 1: 6-(((benzyloxy)carbonyl)amino)hexanoic acid (2.0 g, 1 Eq, 7.5 mmol), and 3-bromopropan-1-amine (1.6 g, 1.5 Eq, 12 mmol) were treated with HATU in a manner similar to Example 104, Step 1 to provide benzyl (6-((3-bromopropyl)amino)-6-oxohexyl)carbamate (1.8 g, 3.3 mmol, 43%) as a yellow solid. [M++]=385.1.

Steps 2-3: tert-Butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (100 mg, 1 Eq, 146 μmol) was treated with benzyl (6-((3-bromopropyl)amino)-6-oxohexyl)carbamate in a manner similar to Example 98, Step 1 then treated with hydrogen and Pd/C in a manner similar to Example 1, Step 7 to provide tert-butyl (1R,3S,5r,7r)-2-(1-(4-(1-(3-(6-aminohexanamido)propyl)piperidin-4-yl)-2-isopropylphenyl)-5-(2,6-dimethoxyphenyl)-1H-pyrazole-3-carboxamido)adamantane-2-carboxylate (45 mg, 53 mol, 77%) as a white solid.

Steps 4-6: 2,2′,2″-(10-(2-(((S)-1-((6-((3-(4-(4-(3-(((1R,3S,5r,7r)-2-Carboxyadamantan-2-yl)carbamoyl)-5-(2,6-dimethoxyphenyl)-1H-pyrazol-1-yl)-3-isopropylphenyl)piperidin-1-yl)propyl)amino)-6-oxohexyl)amino)-5-((3-(2-nitro-1H-imidazol-1-yl)propyl)amino)-1,5-dioxopentan-2-yl)amino)-2-oxoethyl)-1,4,7,10-tetraazacyclododecane-1,4,7-triyl)triacetic acid was synthesized from tert-butyl (1R,3S,5r,7r)-2-(1-(4-(1-(3-(6-aminohexanamido)propyl)piperidin-4-yl)-2-isopropylphenyl)-5-(2,6-dimethoxyphenyl)-1H-pyrazole-3-carboxamido)adamantane-2-carboxylate in a manner similar to Example 106, Steps 3-5 to provide the 2,2,2-trifluoroacetic acid salt of the product (6.6 mg, 4.0 μmol, 17%) as a white solid. MS: Calc'd for C₇₃H₁₀₅N₁₅O₁₇: 1463.78 found [M+H]⁺: 1464.4.

### Example 108: Compound 108

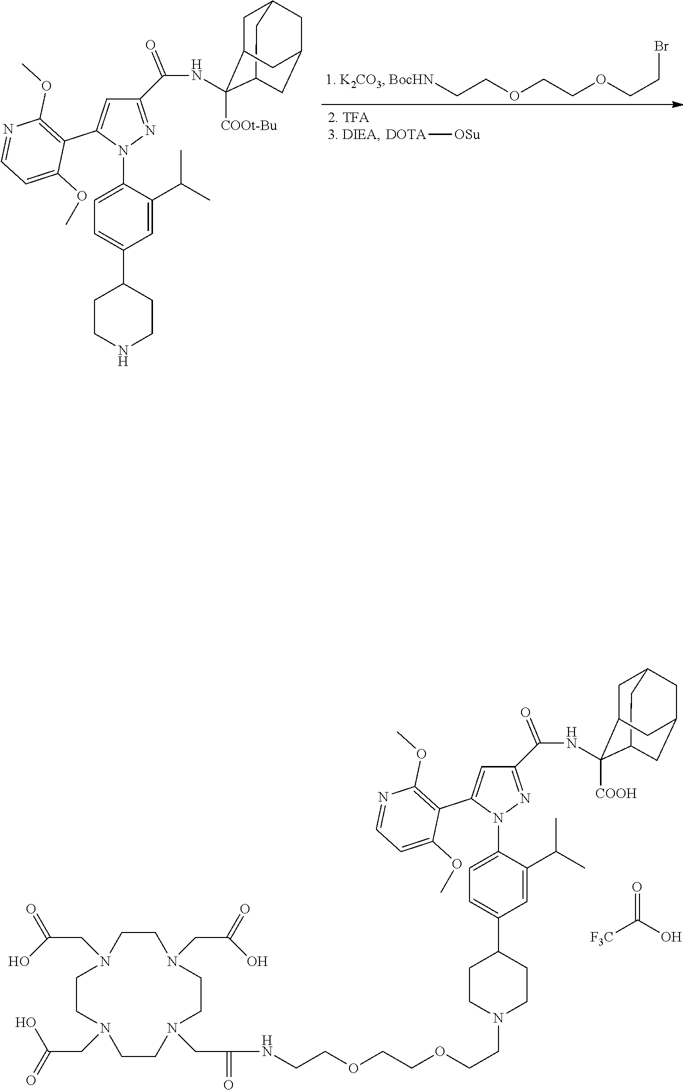

2,2′,2″-(10-(2-((2-(2-(2-(4-(4-(3-(((1R,3S,5r,7r)-2-carboxyadamantan-2-yl)carbamoyl)-5-(2,4-dimethoxypyridin-3-yl)-11H-pyrazol-1-yl)-3-isopropylphenyl)piperidin-1-yl)ethoxy)ethoxy)ethyl)amino)-2-oxoethyl)-1,4,7,10-tetraazacyclododecane-1,4,7-triyl)triacetic acid was synthesized from 6 tert-butyl (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylate (65 mg, 1 Eq, 95 μmol) and tert-butyl (2-(2-(2-bromoethoxy)ethoxy)ethyl)carbamate (47 mg, 1.6 Eq, 0.15 mmol) in a manner similar to Example 98 to provide the 2,2,2-trifluoroacetic acid salt of the product (26.2 mg, 20.8 μmol, 53%) as a white solid. MS: Calc'd for C₅₈H₈₄N₁₀O₁₄: 1144.62, found [M+H]⁺: 1145.6.

### Example 109: Compound 109

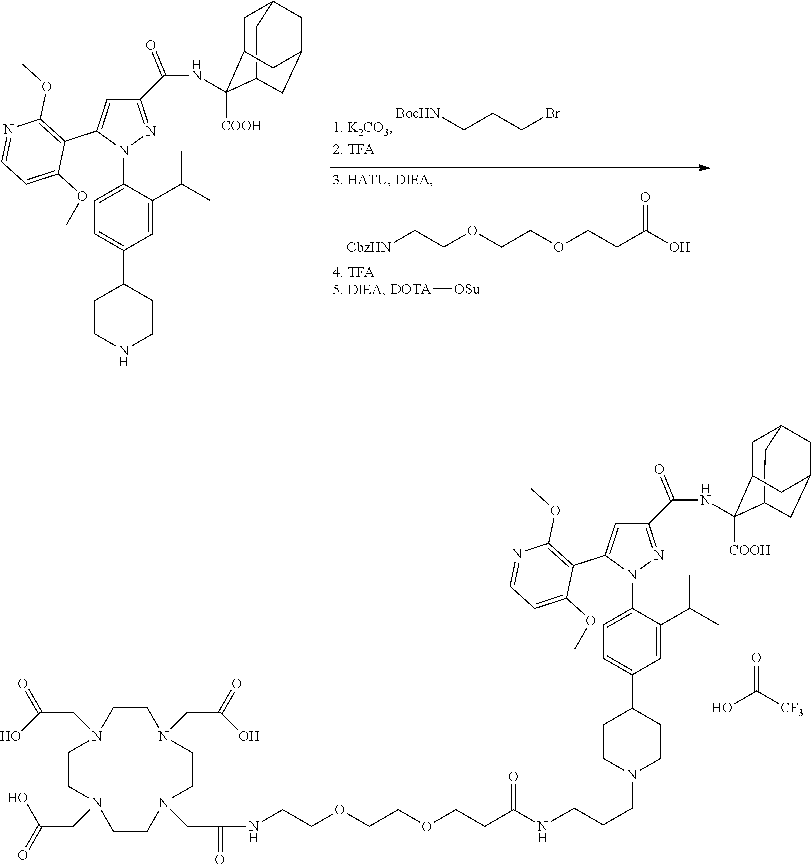

2,2′,2″-O-(16-(4-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)piperidin-1-yl)-2,12-dioxo-6,9-dioxa-3,13-diazahexadecyl)-1,4,7,10-tetraazacyclododecane-1,4,7-triyl)triacetic acid was synthesized from tert-butyl (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylate (85 mg, 1 Eq, 0.12 mmol) and tert-butyl (3-bromopropyl)carbamate (30 mg, 1.0 Eq, 0.13 mmol) in a manner similar to in a manner similar to Example 105 to provide the 2,2,2-trifluoroacetic acid salt of the product (17.5 mg, 13.0 μmol, 30%) as an off-white solid. MS: Calc'd for C₆₂H₉₂N₁₁O₁₅: 1229.67, found [M+H]⁺: 1230.5.

### Example 110: Compound 110

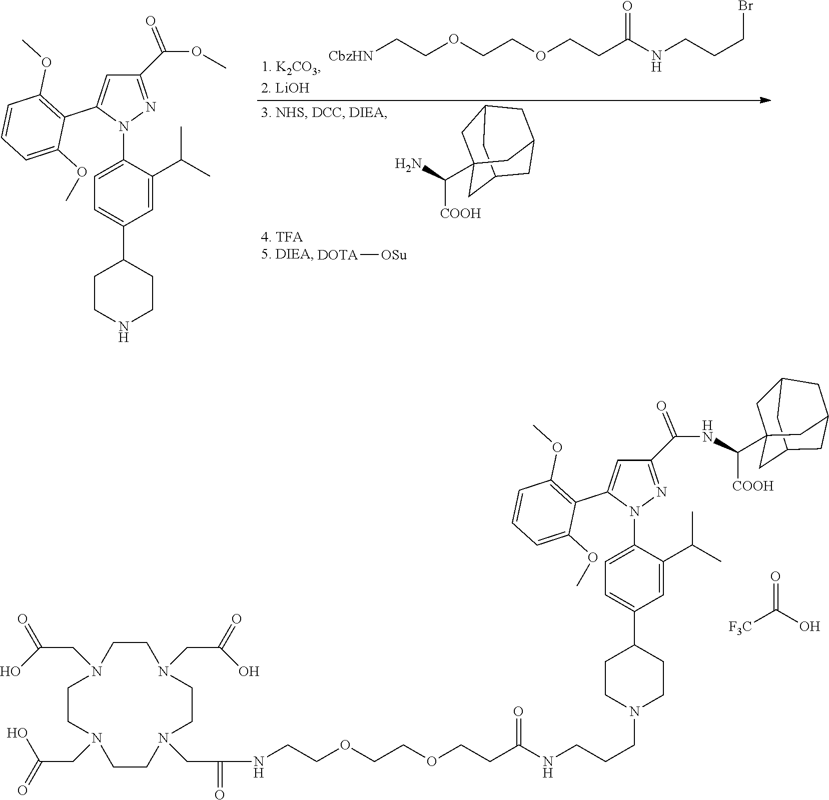

Step 1: Methyl 5-(2,6-dimethoxyphenyl)-1-(2-isopropyl-4-(piperidin-4-yl)phenyl)-1H-pyrazole-3-carboxylate (100 mg, 1 Eq, 216 μmol) and benzyl (2-(2-(3-((3-bromopropyl)amino)-3-oxopropoxy)ethoxy)ethyl)carbamate (140 mug, 1.50 Eq, 325 μmol) were treated with TBAI and K₂CO₃ in a manner similar to Example 99, Step 1 to provide methyl 5-(2,6-dimethoxyphenyl)-1-(4-(1-(3,13-dioxo-1-phenyl-2,7,10-trioxa-4,14-diazaheptadecan-17-yl)piperidin-4-yl)-2-isopropylphenyl)-1H-pyrazole-3-carboxylate (100 mg, 123 μmol, 57.0%) as a yellow oil. [M+H]⁺=814.3.

Step 2: Methyl 5-(2,6-dimethoxyphenyl)-1-(4-(1-(3,13-dioxo-1-phenyl-2,7,10-trioxa-4,14-diazaheptadecan-17-yl)piperidin-4-yl)-2-isopropylphenyl)-1H-pyrazole-3-carboxylate was treated with LiOH in a manner similar to Example 1, Step 4 to provide 5-(2,6-dimethoxyphenyl)-1-(4-(1-(3,13-dioxo-1-phenyl-2,7,10-trioxa-4,14-diazaheptadecan-17-yl)piperidin-4-yl)-2-isopropylphenyl)-1H-pyrazole-3-carboxylic acid (80 mg, 0.10 mmol, 81%) as a light yellow solid, which was used directly in the next step without any purification. [M+H]⁺=800.4.

Step 3: 5-(2,6-Dimethoxyphenyl)-1-(4-(1-(3,13-dioxo-1-phenyl-2,7,10-trioxa-4,14-diazaheptadecan-17-yl)piperidin-4-yl)-2-isopropylphenyl)-1H-pyrazole-3-carboxylic acid was treated with (2S)-2-((3R,5R)-adamantan-1-yl)-2-aminoacetic acid in a manner similar to Example 103, Step 3 to provide (2S)-2-((³R,5R)-adamantan-1-yl)-2-(5-(2,6-dimethoxyphenyl)-1-(4-(1-(3,13-dioxo-1-phenyl-2,7,10-trioxa-4,14-diazaheptadecan-17-yl)piperidin-4-yl)-2-isopropylphenyl)-1H-pyrazole-3-carboxamido)acetic acid (60 mg, 61 μmol, 61%) as a white solid.

Steps 4-5: (2S)-2-((3R,5R)-Adamantan-1-yl)-2-(5-(2,6-dimethoxyphenyl)-1-(4-(1-(3,13-dioxo-1-phenyl-2,7,10-trioxa-4,14-diazaheptadecan-17-yl)piperidin-4-yl)-2-isopropylphenyl)-1H-pyrazole-3-carboxamido)acetic acid was treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(16-(4-(4-(3-(((1S)-((3R,5R)-adamantan-1-yl)(carboxy)methyl)carbamoyl)-5-(2,6-dimethoxyphenyl)-1H-pyrazol-1-yl)-3-isopropylphenyl)piperidin-1-yl)-2,12-dioxo-6,9-dioxa-3,13-diazahexadecyl)-1,4,7,10-tetraazacyclododecane-1,4,7-triyl)triacetic acid (32.1 mg, 23.6 μmol, 42%) as a white solid. MS: Calc'd for C₆₄H₉₅N₁₀O₁₅: 1242.69, found [M+H]⁺: 1243.5.

### Example 111: Compound 111

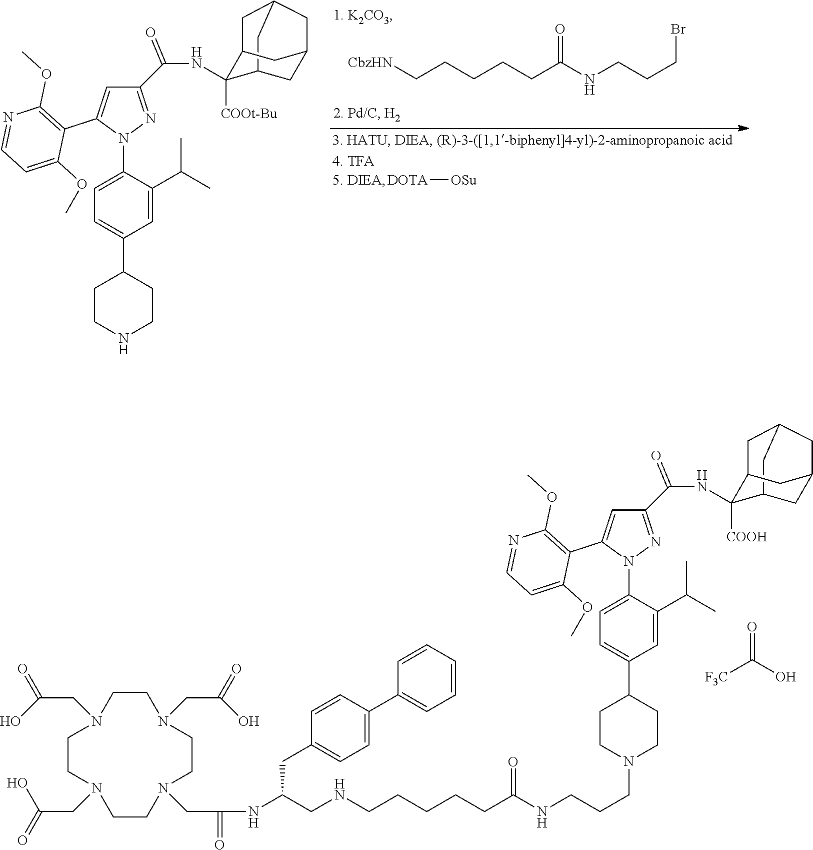

Step 1: tert-Butyl (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylate (100 mg, 1 Eq, 146 kmol) and benzyl (6-((3-bromopropyl)amino)-6-oxohexyl)carbamate (113 mg, 2 Eq, 292 μmol) are treated with K₂CO₃ in a manner similar to Example 84, Step 1 to provide tert-butyl (1R,3 S,5r,7r)-2-(1-(4-(1-(3-(6-(((benzyloxy)carbonyl)amino)hexanamido)propyl)piperidin-4-yl)-2-isopropylphenyl)-5-(2,4-dimethoxypyridin-3-yl)-1H-pyrazole-3-carboxamido)adamantane-2-carboxylate (50 mg, 51 μmol, 35%) as a white solid. [M+H]⁺=988.5.

Step 2: tert-Butyl (1R,3S,5r,7r)-2-(1-(4-(1-(3-(6-(((benzyloxy)carbonyl)amino)hexanamido)propyl)piperidin-4-yl)-2-isopropylphenyl)-5-(2,4-dimethoxypyridin-3-yl)-1H-pyrazole-3-carboxamido)adamantane-2-carboxylate was treated with H₂ and Pd/C and MeOH in a manner similar to Example 1, Step 7 to provide tert-Butyl (1R,3 S,5r,7r)-2-(1-(4-(1-(3-(6-aminohexanamido)propyl)piperidin-4-yl)-2-isopropylphenyl)-5-(2,4-dimethoxypyridin-3-yl)-1H-pyrazole-3-carboxamido)adamantane-2-carboxylate (40 mg, 47 μmol, 93%) as a white solid. [M+H]⁺=:854.7.

Steps 3-5: 2,2′,2″-(10-(2-(((2R)-3-([1,1′-Biphenyl]-4-yl)-1-((6-((3-(4-(4-(3-(((1R,2r,3 S,5r)-2-carboxyadamantan-2-yl)carbamoyl)-5-(2,4-dimethoxypyridin-3-yl)-1H-pyrazol-1-yl)-3-isopropylphenyl)piperidin-1-yl)propyl)amino)-6-oxohexyl)amino)-1-oxopropan-2-yl)amino)-2-oxoethyl)-1,4,7,10-tetraazacyclododecane-1,4,7-triyl)triacetic acid was synthesized from tert-butyl (1R,3S,5r,7r)-2-(1-(4-(1-(3-(6-aminohexanamido)propyl)piperidin-4-yl)-2-isopropylphenyl)-5-(2,4-dimethoxypyridin-3-yl)-1H-pyrazole-3-carboxamido)adamantane-2-carboxylate (40 mg, 1 Eq, 47 mol) and (R)-3-([1,1′-biphenyl]-4-yl)-2-((tert-butoxycarbonyl)amino)propanoic acid (19 mg, 1.2 Eq, 56 μmol) in a manner similar to Example 104, Steps 3-5 to provide the 2,2,2-trifluoroacetic acid salt of the product (22.7 mug, 14.9 μmol, 63%) as a white solid. MS: Calc'd for C₇₆H₁₀₂N₁₂O₁₄: 1406.76, found [M+H]⁺: 1407.7.

### Example 112: Compound 112

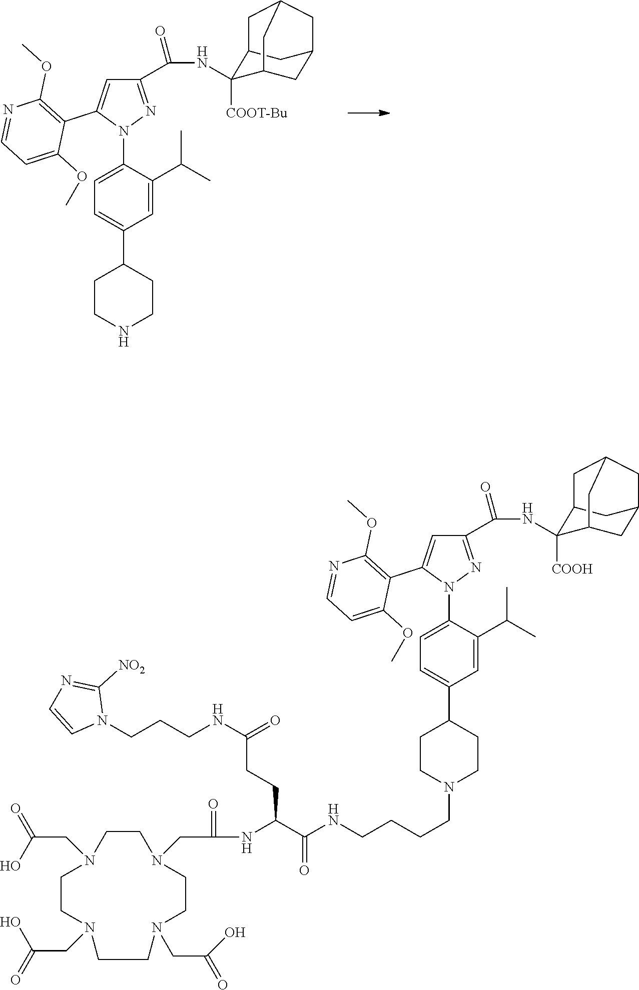

2,2′,2″-(10-(2-(((2S)-1-((4-(4-(4-(3-(((1R,3 S,5r,7r)-2-carboxyadamantan-2-yl)carbamoyl)-5-(2,4-dimethoxypyridin-3-yl)-1H-pyrazol-1-yl)-3-isopropylphenyl)piperidin-1-yl)butyl)amino)-5-((3-(2-nitro-1H-imidazol-1-yl)propyl)amino)-1,5-dioxopentan-2-yl)amino)-2-oxoethyl)-1,4,7,10-tetraazacyclododecane-1,4,7-triyl)triacetic acid was synthesized from tert-butyl (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylate in a manner similar to Example 106 to provide the 2,2,2-trifluoroacetic acid salt of the product (14.9 mg, 10.1 μmol, 25%) as a white solid. MS: Calc'd for C₆₇H₉₅N₁₅O₁₆: 1365.71, found [M+H]⁺: 1366.8.

### Example 113: Compound 113

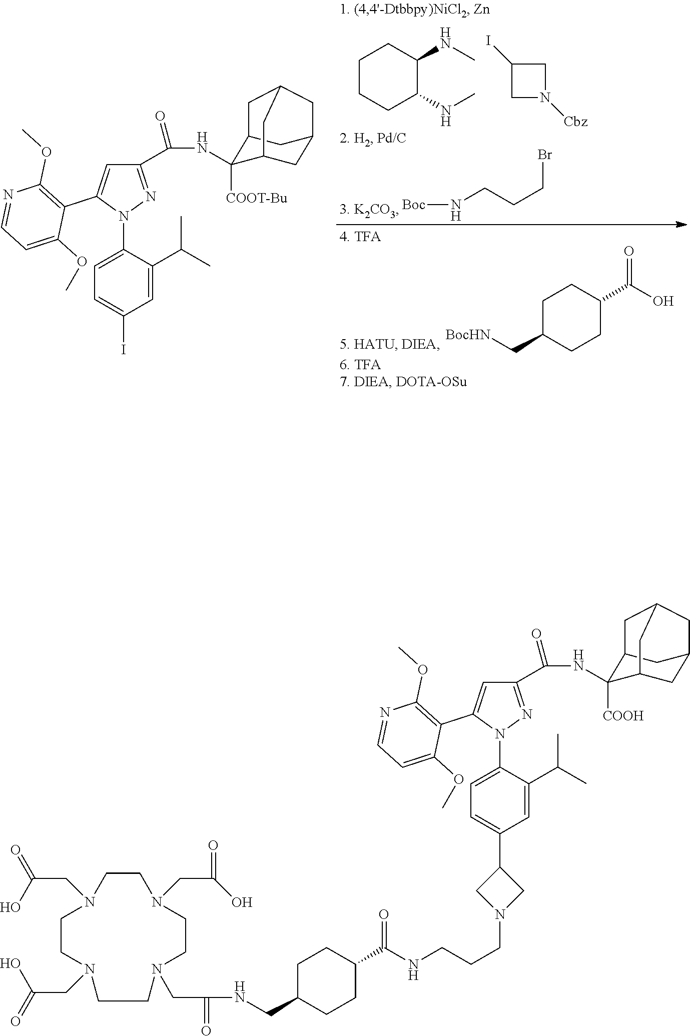

Step 1: A mixture of tert-butyl (1R,3S,5r,7r)-2-(5-(2,4-dimethoxypyridin-3-yl)-1-(4-iodo-2-isopropylphenyl)-1H-pyrazole-3-carboxamido)adamantane-2-carboxylate(470 mg, 1 Eq, 647 μmol), [4,4′-Bis(1,1-dimethylethyl)-2,2′-bipyridine]nickel (II) dichloride (64.4 mg, 0.250 Eq, 162 μmol), (1R,2R)—N1,N2-dimethylcyclohexane-1,2-diamine (46.0 mg, 0.500 Eq, 323 μmol), benzyl 3-iodoazetidine-1-carboxylate (410 mg, 2.00 Eq, 10.29 mmol) (from Example 83, Step 6) and DMAc (6 mL) was stirred at 60° C. for 16 hours, then zinc (148 mg, 3.50 Eq, 2.26 mmol) was added and the reaction mixture was stirred at 60° C. for an additional 1 hour. The mixture was directly purified by MPLC to afford benzyl 3-(4-(3-(((1R,3S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)azetidine-1-carboxylate (170 mg, 215 μmol, 33.3%) as a yellow oil. [M+H]⁺=790.4.

Step 2: Benzyl 3-(4-(3-(((1R,3S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)azetidine-1-carboxylate was treated with hydrogen and Pd/C in a manner similar to Example 1, Step 7 to provide tert-butyl (1R,3 S,5r,7r)-2-(1-(4-(azetidin-3-yl)-2-isopropylphenyl)-5-(2,4-dimethoxypyridin-3-yl)-1-H-pyrazole-3-carboxamido)adamantane-2-carboxylate (120 mg, 183 μmol, 85.0%) as a yellow oil. [M+H]⁺=656.4.

Steps 3-4: tert-Butyl (1R,3S,5r,7r)-2-(1-(4-(azetidin-3-yl)-2-isopropylphenyl)-5-(2,4-dimethoxypyridin-3-yl)-1H-pyrazole-3-carboxamido)adamantane-2-carboxylate was treated with K₂CO₃ and tert-butyl (3-bromopropyl)carbamate in a manner similar to Example 58, Step 2 then treated with TFA in a manner similar to Example 1, Step 8 to provide (1R,3S,5r,7r)-2-(1-(4-(1-(3-aminopropyl)azetidin-3-yl)-2-isopropylphenyl)-5-(2,4-dimethoxypyridin-3-yl)-1H-pyrazole-3-carboxamido)adamantane-2-carboxylic acid (40 mg, 55 μmol, 99%), which was directly used in the next step without any purification.

Step 5: A mixture of (1r,4r)-4-(((tert-butoxycarbonyl)amino)methyl)cyclohexane-1-carboxylic acid (21 mg, 1.5 Eq, 82 μmol), HATU (31 mg, 1.5 Eq, 82 μmol), DIEA (21 mg, 3.0 Eq, 0.16 mmol) and DMF (1.5 mL) was stirred at 20° C. for 10 minutes, then (1R,3S,5r,7r)-2-(1-(4-(1-(3-aminopropyl)azetidin-3-yl)-2-isopropylphenyl)-5-(2,4-dimethoxypyridin-3-yl)-1H-pyrazole-3-carboxamido)adamantane-2-carboxylic acid (40 mg, 90% Wt, 1 Eq, 55 μmol) was added and the reaction mixture was stirred at 25° C. for an additional 1 hour. The mixture was directly purified by MPLC to provide (1R,3S,5r,7r)-2-(1-(4-(1-(3-((1r,4r)-4-(((tert-butoxycarbonyl)amino)methyl)cyclohexane-1-carboxamido)propyl)azetidin-3-yl)-2-isopropylphenyl)-5-(2,4-dimethoxypyridin-3-yl)-1H-pyrazole-3-carboxamido)adamantane-2-carboxylic acid (35 mg, 39 μmol, 71%) as a yellow oil. [M+H−Boc]⁺=796.4.

Steps 6-7: (1R,3S,5r,7r)-2-(1-(4-(1-(3-((1r,4r)-4-(((tert-Butoxycarbonyl)amino)methyl)cyclohexane-1-carboxamido)propyl)azetidin-3-yl)-2-isopropylphenyl)-5-(2,4-dimethoxypyridin-3-yl)-1H-pyrazole-3-carboxamido)adamantane-2-carboxylic acid was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((((1r,4r)-4-((3-(3-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)propyl)carbamoyl)cyclohexyl)methyl)amino)-2-oxoethyl)-1,4,7,10-tetraazacyclododecane-1,4,7-triyl)triacetic acid (16.7 mg, 12.9 μmol, 40%) as a white solid. MS: Calc'd for C₆₁H₈₇N₁₁O₁₃: 1181.65, found [M+H]⁺: 1182.7.

### Example 114: Compound 114

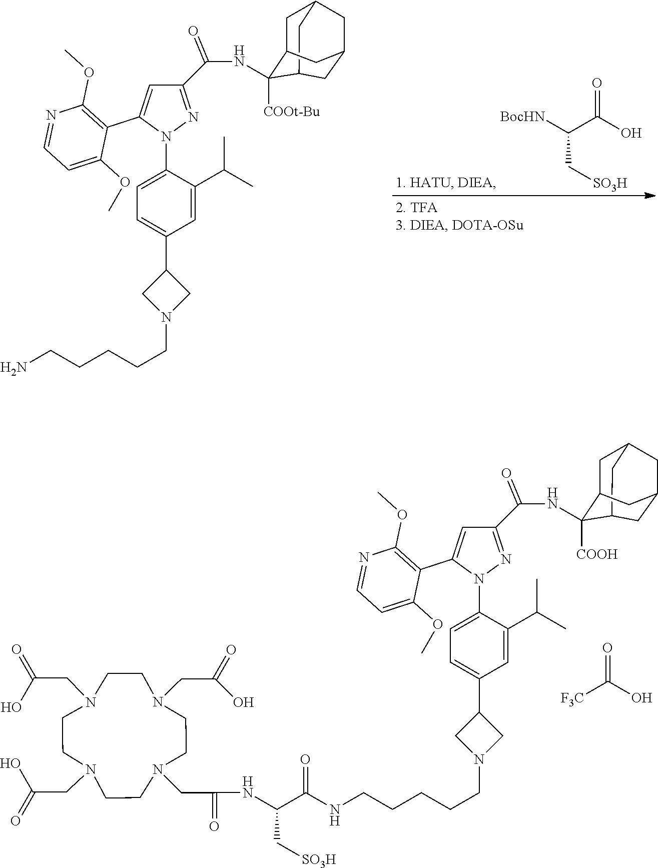

2,2′,2″-(10-(2-(((2R)-1-((5-(3-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)pentyl)amino)-1-oxo-3-sulfopropan-2-yl)amino)-2-oxoethyl)-1,4,7,10-tetraazacyclododecane-1,4,7-triyl)triacetic acid was synthesized from tert-butyl (1R,3S,5r,7r)-2-(1-(4-(1-(5-aminopentyl)azetidin-3-yl)-2-isopropylphenyl)-5-(2,4-dimethoxypyridin-3-yl)-1H-pyrazole-3-carboxamido)adamantane-2-carboxylate (40 mg, 1 Eq, 54 μmol) and (tert-butoxycarbonyl)(sulfo)-D-alanine (17 mg, 1.2 Eq, 63 μmol) in a manner similar to Compound 106, Steps 3-5 to provide the 2,2,2-trifluoroacetic acid salt of the product (14.5 mg, 10.8 μmol, 39%) as a white solid. MS: Calc'd for C₅₈H₃N₁₁O₁₆S: 1221.57, found [M+H]⁺: 1222.6.

### Example 115: Compound 115

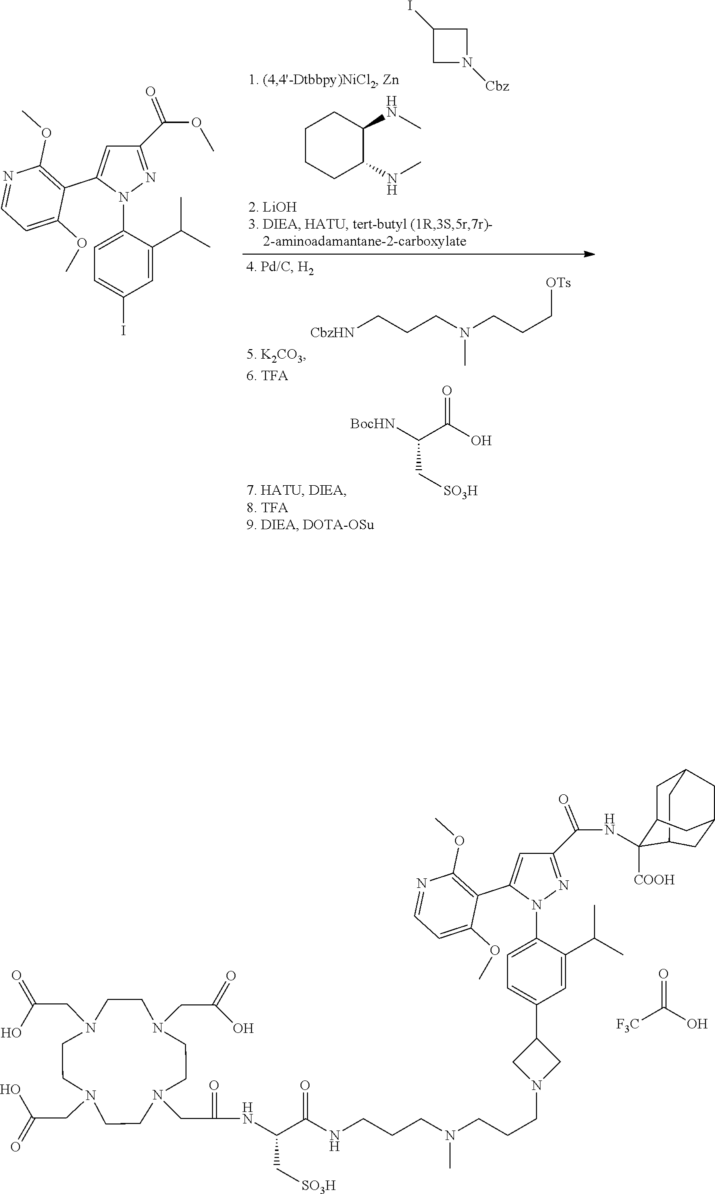

Step 1: Methyl 1-(4-(1-((benzyloxy)carbonyl)azetidin-3-yl)-2-isopropylphenyl)-5-(2,4-dimethoxypyridin-3-yl)-1H-pyrazole-3-carboxylate was synthesized from methyl 5-(2,4-dimethoxypyridin-3-yl)-1-(4-iodo-2-isopropylphenyl)-1H-pyrazole-3-carboxylate (1.55 g, 1 Eq, 3.06 mmol) and benzyl 3-iodoazetidine-1-carboxylate (1.94 g, 2.00 Eq, 6.12 mmol) in a manner similar to Example 113, Step 1 to provide the product (820 mg, 1.44 mmol, 47.0%) as a yellow oil.

Steps 2-3: Methyl 1-(4-(1-((benzyloxy)carbonyl)azetidin-3-yl)-2-isopropylphenyl)-5-(2,4-dimethoxypyridin-3-yl)-1H-pyrazole-3-carboxylate was treated with LiOH in a manner similar to Example 8, Step 4, then treated with tert-butyl (1R,3S,5r,7r)-2-aminoadamantane-2-carboxylate, HATU and DIEA in a manner similar to Example 8, Step 5 to provide benzyl 3-(4-(3-(((1R,3S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)azetidine-1-carboxylate (680 mg, 861 μmol, 76.1%) as an off-yellow oil.

Steps 4-5: Benzyl 3-(4-(3-(((1R,3S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)azetidine-1-carboxylate was treated with hydrogen and Pd/C in a manner similar to Example 1, Step 7 then treated with 3-((3-(((benzyloxy)carbonyl)amino)propyl)(methyl)amino)propyl 4-methylbenzenesulfonate and K₂CO₃ in ACN for 1 hour at 25° C. to provide tert-butyl (1R,3 S,5r,7r)-2-(1-(4-(1-(3-((3-(((benzyloxy)carbonyl)amino)propyl)(methyl)amino)propyl)azetidin-3-yl)-2-isopropylphenyl)-5-(2,4-dimethoxypyridin-3-yl)-1H-pyrazole-3-carboxamido)adamantane-2-carboxylate (48 mg, 52 μmol, 57%) as a light-yellow oil.

Steps 6-9: tert-Butyl (1R,3S,5r,7r)-2-(1-(4-(1-(3-((3-(((benzyloxy)carbonyl)amino)propyl)(methyl)amino)propyl)azetidin-3-yl)-2-isopropylphenyl)-5-(2,4-dimethoxypyridin-3-yl)-1H-pyrazole-3-carboxamido)adamantane-2-carboxylate was treated with TFA in a manner similar to Example 104, Step 2, then treated with (tert-butoxycarbonyl)(sulfo)-D-alanine in a manner similar to Example 103, Step 5 to provide (1R,3 S,5r,7r)-2-(5-(2,4-dimethoxypyridin-3-yl)-1-(2-isopropyl-4-(1-((R)-2,2,12-trimethyl-4,7-dioxo-6-(sulfomethyl)-3-oxa-5,8,12-triazapentadecan-15-yl)azetidin-3-yl)phenyl)-1H-pyrazole-3-carboxamido)adamantane-2-carboxylic acid (24 mg, 25 mol, 46%) as a yellow oil. [M+H]⁺=979.4. The product was treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-(((2R)-1-((3-((3-(3-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)propyl)(methyl)amino)propyl)amino)-1-oxo-3-sulfopropan-2-yl)amino)-2-oxoethyl)-1,4,7,10-tetraazacyclododecane-1,4,7-triyl)triacetic acid (12.8 mg, 9.28 μmol, 34%) as a white solid. MS: Calc'd for C₆₀H₈₈N₁₂O₁₆S: 1264.62, found [M+H]⁺: 1265.5.

### Example 116: Compound 116

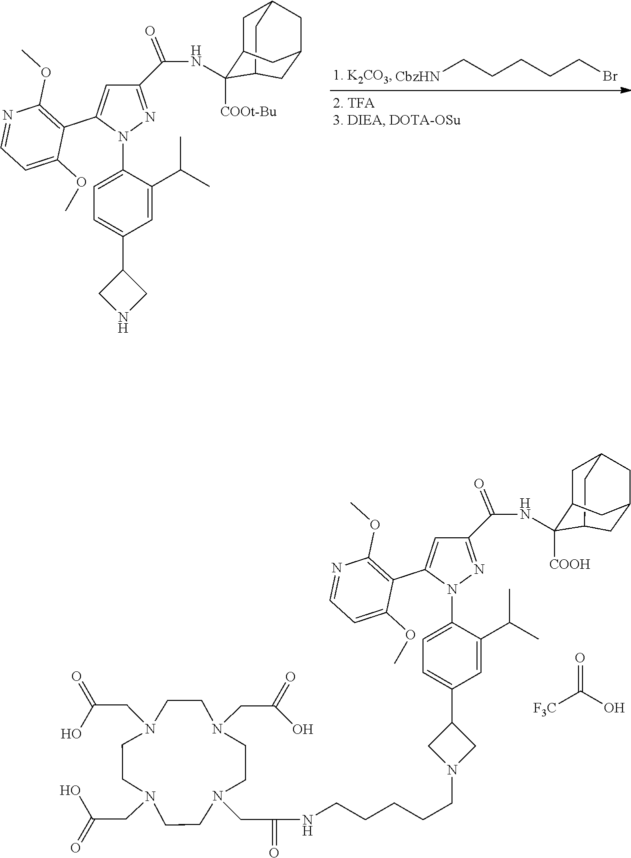

Step 1: To a mixture of tert-butyl (1R,3S,5r,7r)-2-(1-(4-(azetidin-3-yl)-2-isopropylphenyl)-5-(2,4-dimethoxypyridin-3-yl)-1H-pyrazole-3-carboxamido)adamantane-2-carboxylate (70 mg, 1 Eq, 0.11 mmol), benzyl (5-bromopentyl)carbamate (48 mg, 1.5 Eq, 0.16 mmol) and DMF (2 mL), was added K₂CO₃ (18 mg, 1.2 Eq, 0.13 mmol). The reaction mixture was stirred at 80° C. for 1 hour. The mixture was purified by Prep-HPLC to afford tert-butyl (1R,3S,5r,7r)-2-(1-(4-(1-(5-(((benzyloxy)carbonyl)amino)pentyl)azetidin-3-yl)-2-isopropylphenyl)-5-(2,4-dimethoxypyridin-3-yl)-1H-pyrazole-3-carboxamido)adamantane-2-carboxylate (40 mug, 46 μmol, 43%) as a white solid. [M+H]⁺=875.4.

Steps 2-3: tert-Butyl (1R,3 S,5r,7r)-2-(1-(4-(1-(5-(((benzyloxy)carbonyl)amino)pentyl)azetidin-3-yl)-2-isopropylphenyl)-5-(2,4-dimethoxypyridin-3-yl)-1H-pyrazole-3-carboxamido)adamantane-2-carboxylate was treated with TFA in a manner similar to Example 104, Step 2 then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((5-(3-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)pentyl)amino)-2-oxoethyl)-1,4,7,10-tetraazacyclododecane-1,4,7-triyl)triacetic acid (8.4 mg, 7.1 μmol, 16%) as a white solid. MS: Calc'd for C₅₅H₇₈N₁₀O₁₂: 1070.58, found [M+H]⁺:1071.6.

### Synthesis of Compound 116-Lu

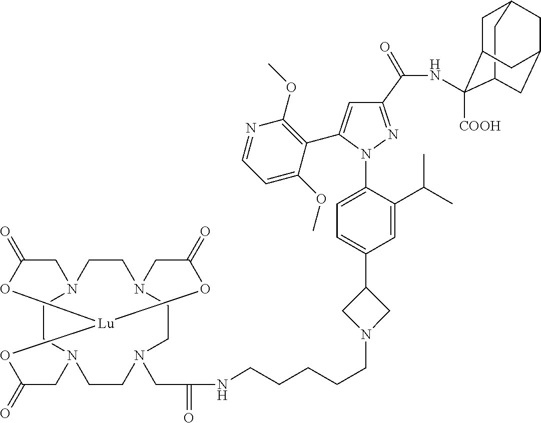

A mixture of 2,2′,2″-(10-(2-((5-(3-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)pentyl)amino)-2-oxoethyl)-1,4,7,10-tetraazacyclododecane-1,4,7-triyl)triacetic acid-2,2,2-trifluoroacetic acid (1/1) (25 mg, 1 Eq, 21 μmol), lutetium(III) chloride (30 mg, 5.1 Eq, 0.11 mmol) and NaOAc/AcOH Buffer (2 mL) was stirred at 80° C. for 30 min. The crude mixture was directly purified by Prep-HPLC to afford the product (21.5 mg, 15.8 μmol, 75%) as a white solid. MS: Calc'd for C₅₅H₇₅LuN₁₀O₁₂: 1242.50, found [M+H]⁺: 1243.5.

### Synthesis of Compound 116-Ga

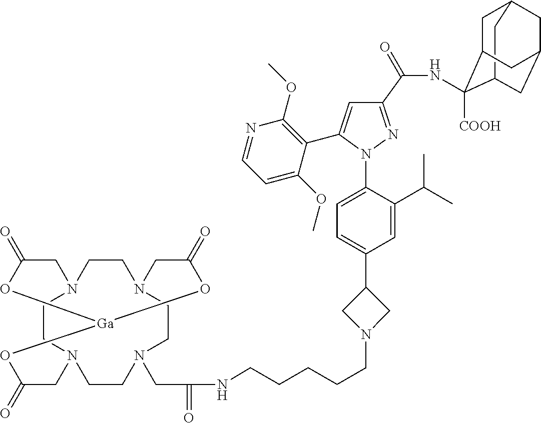

A mixture of 2,2′,2″-(10-(2-((5-(3-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)pentyl)amino)-2-oxoethyl)-1,4,7,10-tetraazacyclododecane-1,4,7-triyl)triacetic acid (25 μg, 1 Eq, 23 μmol), gallium trichloride (12 mg, 2.9 Eq, 68 μmol) and NaOAc/AcOH Buffer (2 mL) was stirred at 80° C. for 2 hours. The crude product was purified by Prep-HPLC to afford the product (15.2 mg, 12.1 μmol, 52%) as a white solid. MS: Calc'd for C₅₅H₇₅GaN₁₀O₁₂: 1136.48, found [M+H]⁺: 1137.6.

### Example 117: Compound 117

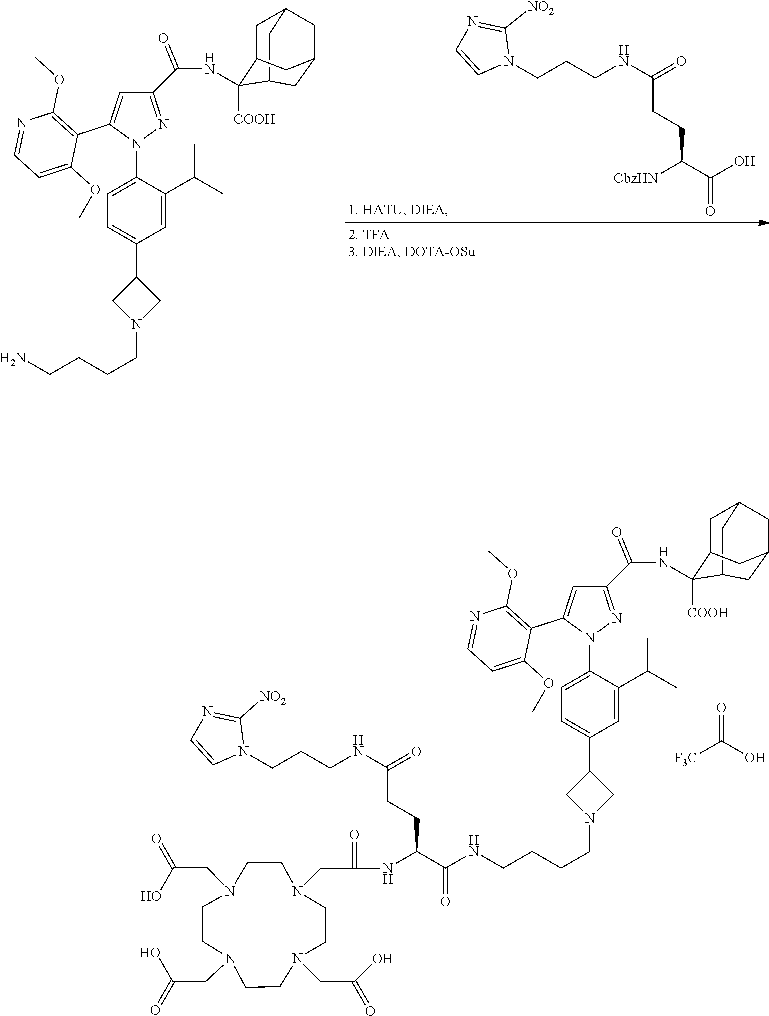 [SMILES: COc1ccnc(OC)c1-c1cc(C(=O)NC2(C(=O)O)C3CC4CC(C3)CC2C4)nn1-c1ccc(C2CN(CCCCN)C2)cc1C(C)C ; O=C(CC[C@H](NC(=O)OCc1ccccc1)C(=O)O)NCCCn1ccnc1[N+](=O)[O-] ; COc1ccnc(OC)c1-c1cc(C(=O)NC2(C(=O)O)C3CC4CC(C3)CC2C4)nn1-c1ccc(C2CN(CCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)C2)cc1C(C)C ; O=C(O)C(F)(F)F]

2,2′,2″-(10-(2-(((2S)-1-((5-(3-(4-(3-(((1R,3 S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)pentyl)amino)-5-((3-(2-nitro-1H-imidazol-1-yl)propyl)amino)-1,5-dioxopentan-2-yl)amino)-2-oxoethyl)-1,4,7,10-tetraazacyclododecane-1,4,7-triyl)triacetic acid was synthesized from tert-butyl (1R,3S,5r,7r)-2-(1-(4-(1-(5-aminopentyl)azetidin-3-yl)-2-isopropylphenyl)-5-(2,4-dimethoxypyridin-3-yl)-1H-pyrazole-3-carboxamido)adamantane-2-carboxylate (70 mg, 1 Eq, 94 μmol) and N2-((benzyloxy)carbonyl)-N5-(3-(2-nitro-1H-imidazol-1-yl)propyl)-L-glutamine (49 mg, 1.2 Eq, 0.11 mmol) in a manner similar to Example 106, Steps 3-5 to provide the 2,2,2-trifluoroacetic acid salt of the product (26.6 mg, 18.1 μmol, 44%) as a white solid. MS: Calc'd for C₆₆H₉₃N₁₅O₁₆: 1351.69, found [M+H]⁺: 1352.6.

### Example 118: Compound 118

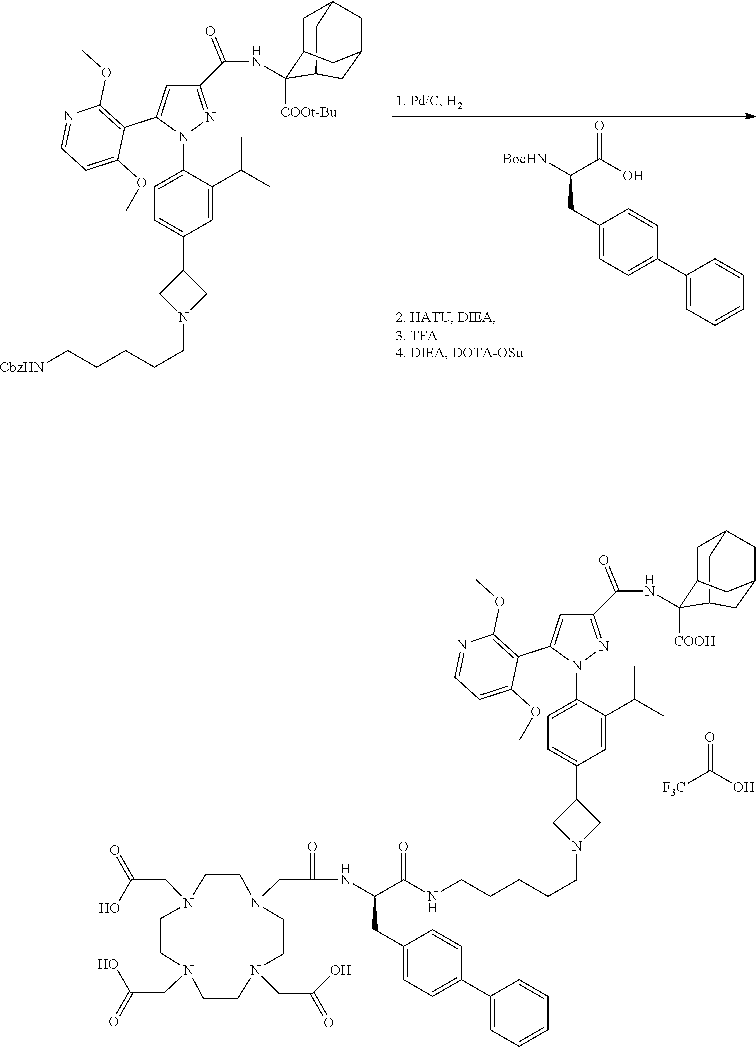

Step 1: tert-Butyl (1R,3S,5r,7r)-2-(1-(4-(1-(5-(((benzyloxy)carbonyl)amino)pentyl)azetidin-3-yl)-2-isopropylphenyl)-5-(2,4-dimethoxypyridin-3-yl)-1H-pyrazole-3-carboxamido)adamantane-2-carboxylate (312 mg, 1 Eq, 357 kmol) was treated with H₂ and Pd/C (29 mg, 8.1 Eq, 0.27 mmol) in a manner similar to Example 1, Step 7 to provide tert-butyl (1R,3S,5r,7r)-2-(1-(4-(1-(5-aminopentyl)azetidin-3-yl)-2-isopropylphenyl)-5-(2,4-dimethoxypyridin-3-yl)-1-pyrazole-3-carboxamido)adamantane-2-carboxylate (160 mg, 216 μmol, 60.6%) as a yellow oil.

Steps 2-4: 2,2′,2″-(10-(2-(((2RS)-3-([1,1′-biphenyl]-4-yl)-1-((5-(3-(4-(3-(((1R,2r,3S,5r)-2-carboxyadamantan-2-yl)carbamoyl)-5-(2,4-dimethoxypyridin-3-yl)-1H-pyrazol-1-yl)-3-isopropylphenyl)azetidin-1-yl)pentyl)amino)-1-oxopropan-2-yl)amino)-2-oxoethyl)-1,4,7,10-tetraazacyclododecane-1,4,7-triyl)triacetic acid was prepared from tert-butyl (1R,3 S,5r,7r)-2-(1-(4-(1-(5-aminopentyl)azetidin-3-yl)-2-isopropylphenyl)-5-(2,4-dimethoxypyridin-3-yl)-1H-pyrazole-3-carboxamido)adamantane-2-carboxylate and (R)-3-([1,1′-biphenyl]-4-yl)-2-((tert-butoxycarbonyl)amino)propanoic acid in a manner similar to Example 106, Steps 3-5 to provide the 2,2,2-trifluoroacetic acid salt of the product (4.9 mg, 3.5 μmol, 21%) as a white solid. MS: Calc'd for C₇₀H₉₁N₁₁O₁₃: 1293.68, found [M+H]⁺: 1294.6.

### Example 119: Compound 119

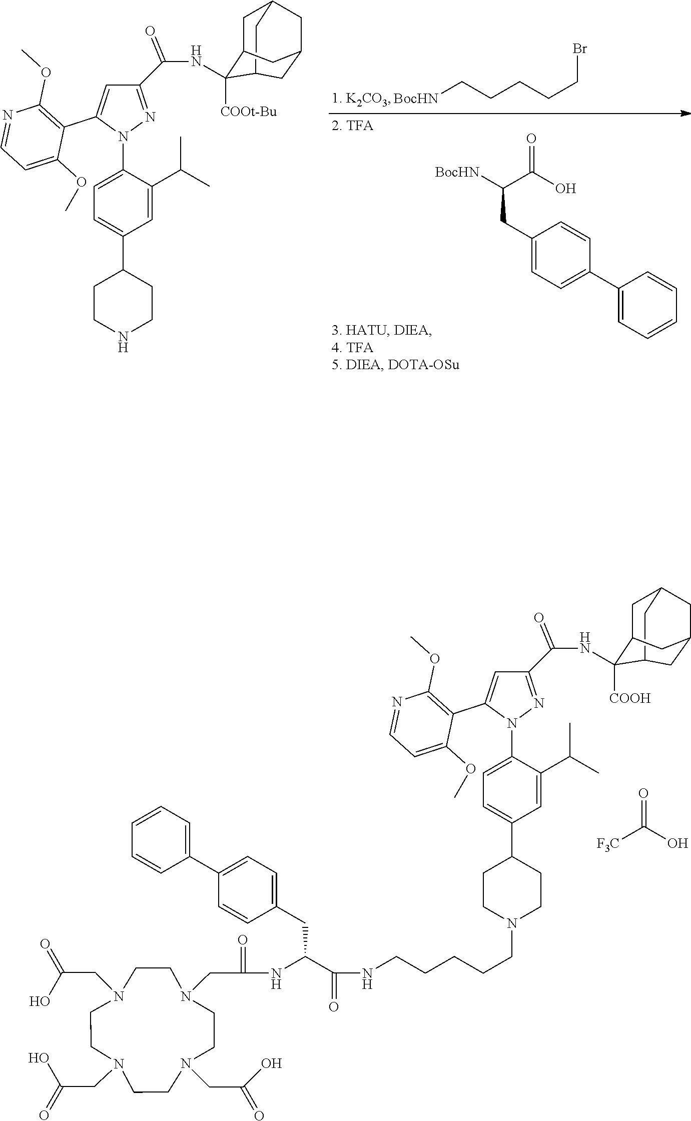

Steps 1-2: tert-Butyl (1R,3S,5r,7r)-2-(5-(2,4-dimethoxypyridin-3-yl)-1-(2-isopropyl-4-(piperidin-4-yl)phenyl)-1H-pyrazole-3-carboxamido)adamantane-2-carboxylate (85 mg, 1 Eq, 0.12 mmol) was treated with benzyl (5-bromopentyl)carbamate (99 mg, 3.0 Eq, 0.37 mmol) in a manner similar to Example 98, Step 1 then treated with TEA in a manner similar to Example 104, Step 2 to provide (1R,3S,5r,7r)-2-(1-(4-(1-(5-aminopentyl)piperidin-4-yl)-2-isopropylphenyl)-5-(2,4-dimethoxypyridin-3-yl)-1H-pyrazole-3-carboxamido)adamantane-2-carboxylic acid (60 mg, 72 μmol, 100%) as a yellow crude oil, which was used for next step without any purification.

Steps 3-5: 2,2′,2″-(10-(2-(((2R)-3-([1,1′-biphenyl]-4-yl)-1-((5-(4-(4-(3-(((1R,2r,3 S,5r)-2-carboxyadamantan-2-yl)carbamoyl)-5-(2,4-dimethoxypyridin-3-yl)-1H-pyrazol-1-yl)-3-isopropylphenyl)piperidin-1-yl)pentyl)amino)-1-oxopropan-2-yl)amino)-2-oxoethyl)-1,4,7,10-tetraazacyclododecane-1,4,7-triyl)triacetic acid was prepared from (1R,3S,5r,7r)-2-(1-(4-(1-(5-aminopentyl)piperidin-4-yl)-2-isopropylphenyl)-5-(2,4-dimethoxypyridin-3-yl)-1H-pyrazole-3-carboxamido)adamantane-2-carboxylic acid and (R)-3-([1,1′-biphenyl]-4-yl)-2-((tert-butoxycarbonyl)amino)propanoic acid in a manner similar to Example 106, Steps 3-5 to provide the 2,2,2-trifluoroacetic acid salt of the product (29.3 mg, 20.4 μmol, 76%) as white solid. MS: Calc'd for C₇₂H₉₅N₁₁O₁₃: 1321.71, found [M+H]⁺: 1322.6.

### Example 120: Compound 120

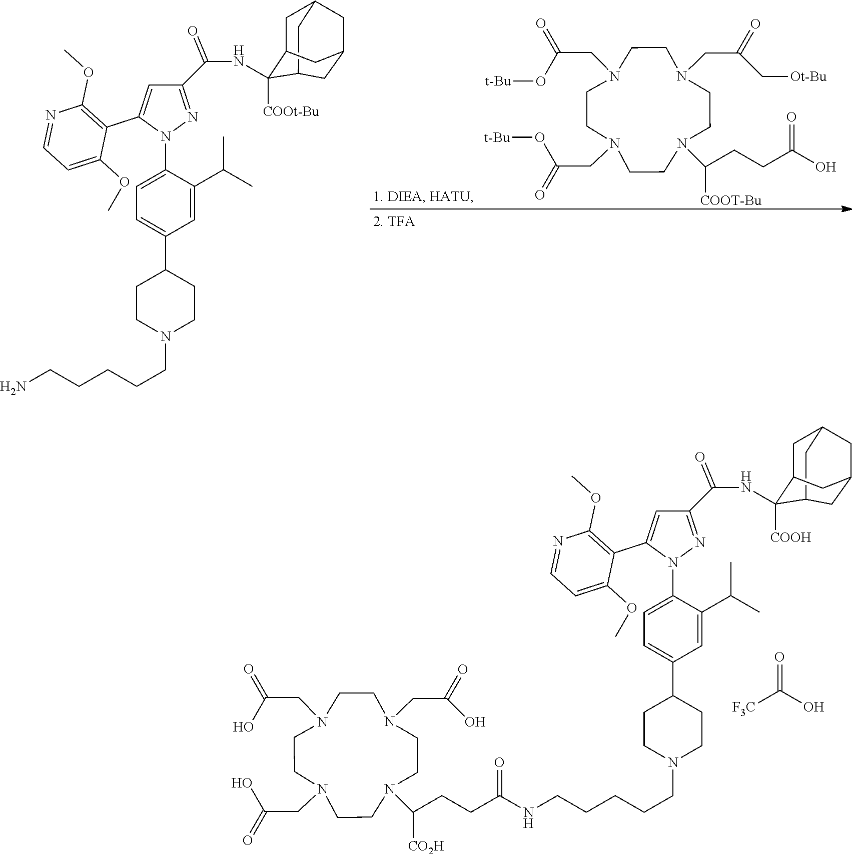

Step 1: A mixture of 5-(tert-butoxy)-5-oxo-4-(4,7,10-tris(2-(tert-butoxy)-2-oxoethyl)-1,4,7,10-tetraazacyclododecan-1-yl)pentanoic acid (68 mg, 1.5 Eq, 98 μmol), HATU (27 mg, 1.1 Eq, 72 μmol), DIEA (34 μL, 3.0 Eq, 200 μmol) and DMF (2 mL) was stirred at 25° C. for 10 minutes, then tert-butyl (1R,3S,5r,7r)-2-(1-(4-(1-(5-aminopentyl)piperidin-4-yl)-2-isopropylphenyl)-5-(2,4-dimethoxypyridin-3-yl)-1H-pyrazole-3-carboxamido)adamantane-2-carboxylate (50 mg, 1 Eq, 65 μmol) was added and the reaction mixture was stirred at 25° C. for an additional 1 hour. The mixture was directly purified by MPLC to afford tri-tert-butyl 2,2′,2″-(10-(1-(tert-butoxy)-5-((5-(4-(4-(3-(((1R,3 S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)piperidin-1-yl)pentyl)amino)-1,5-dioxopentan-2-yl)-1,4,7,10-tetraazacyclododecane-1,4,7-triyl)triacetate (20 mg, 14 μmol, 21%) as a white solid.

Step 2: To a mixture of tri-tert-butyl 2,2′,2″-(10-(1-(tert-butoxy)-5-((5-(4-(4-(3-(((1R,3S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)piperidin-1-yl)pentyl)amino)-1,5-dioxopentan-2-yl)-1,4,7,10-tetraazacyclododecane-1,4,7-triyl)triacetate (20 mg, 1 Eq, 14 μmol) and DCM (1 mL) was added TFA (2 mL). The reaction mixture was stirred at 25° C. for 1 hour. The crude product was purified by Prep-HPLC to afford the 2,2,2-trifluoroacetic acid salt of 2,2′,2″-(10-(1-carboxy-4-((5-(4-(4-(3-((((1R,3S,5r,7r)-2-carboxyadamantan-2-yl)carbamoyl)-5-(2,4-dimethoxypyridin-3-yl)-1H-pyrazol-1-yl)-3-isopropylphenyl)piperidin-1-yl)pentyl)amino)-4-oxobutyl)-1,4,7,10-tetraazacyclododecane-1,4,7-triyl)triacetic acid (6.1 mg, 4.7 μmol, 34%) as a white solid. MS: Calc'd for C₆₀H₈₆N₁₀O₄: 1170.63, found [M+H]⁺: 1171.6.

### Example 121: Compound 121

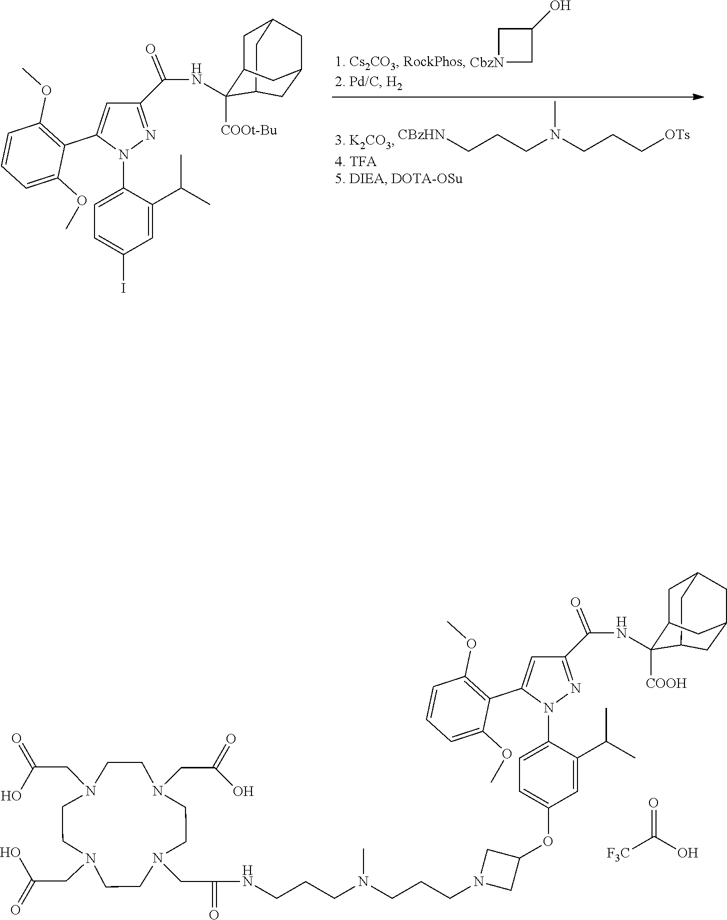

Step 1: A mixture of tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (310 mg, 1 Eq, 427 μmol), Cs₂CO₃ (418 mg, 3.00 Eq, 1.28 mmol), 2-(di-t-butylphosphino)-3-methoxy-6-methyl-2′-4′-6′-tri-i-propyl-1,1′-biphenyl (40.0 mg, 0.200 Eq, 85.3 μmol), benzyl 3-hydroxyazetidine-1-carboxylate (133 mg, 1.50 Eq, 642 μmol) and toluene (5 mL) under N₂ was stirred at 80° C. for 1 hour. The mixture was directly purified by MPLC to afford benzyl 3-(4-(5-(2,6-dimethoxyphenyl)-3-(((1R,3S,5r,7r)-2-(pivaloyloxy)adamantan-2-yl)carbamoyl)-1H-pyrazol-1-yl)-3-isopropylphenoxy)azetidine-1-carboxylate (220 mg, 273 μmol, 64.0%) as a white solid.

Step 2: Benzyl 3-(4-(5-(2,6-dimethoxyphenyl)-3-(((1R,3S,5r,7r)-2-(pivaloyloxy)adamantan-2-yl)carbamoyl)-1H-pyrazol-1-yl)-3-isopropylphenoxy)azetidine-1-carboxylate was treated with H₂ and Pd/C in a manner similar to Example 1, Step 7 to provide (1R,3 S,5r,7r)-2-(1-(4-(azetidin-3-yloxy)-2-isopropylphenyl)-5-(2,6-dimethoxyphenyl)-1H-pyrazole-3-carboxamido)adamantan-2-yl pivalate as white solid. [M+H]⁺=671.6.

Steps 3-5: 2,2′,2″-(10-(2-((3-((3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenoxy)azetidin-1-yl)propyl)(methyl)amino)propyl)amino)-2-oxoethyl)-1,4,7,10-tetraazacyclododecane-1,4,7-triyl)triacetic acid was prepared from (1R,3S,5r,7r)-2-(1-(4-(azetidin-3-yloxy)-2-isopropylphenyl)-5-(2,6-dimethoxyphenyl)-1H-pyrazole-3-carboxamido)adamantan-2-yl pivalate and 3-((3-(((benzyloxy)carbonyl)amino)propyl)(methyl)amino)propyl 4-methylbenzenesulfonate in manner similar to Example 50, Steps 3-5 to provide the trifluoroacetic acid salt of the product (16.3 mg, 13.1 μmol, 28%) as a white solid. MS: Calc'd for C₅₈H₈₄N₁₀O₁₃: 1128.62, found [M+H]⁺: 1129.5.

### Example 122: Compound 122

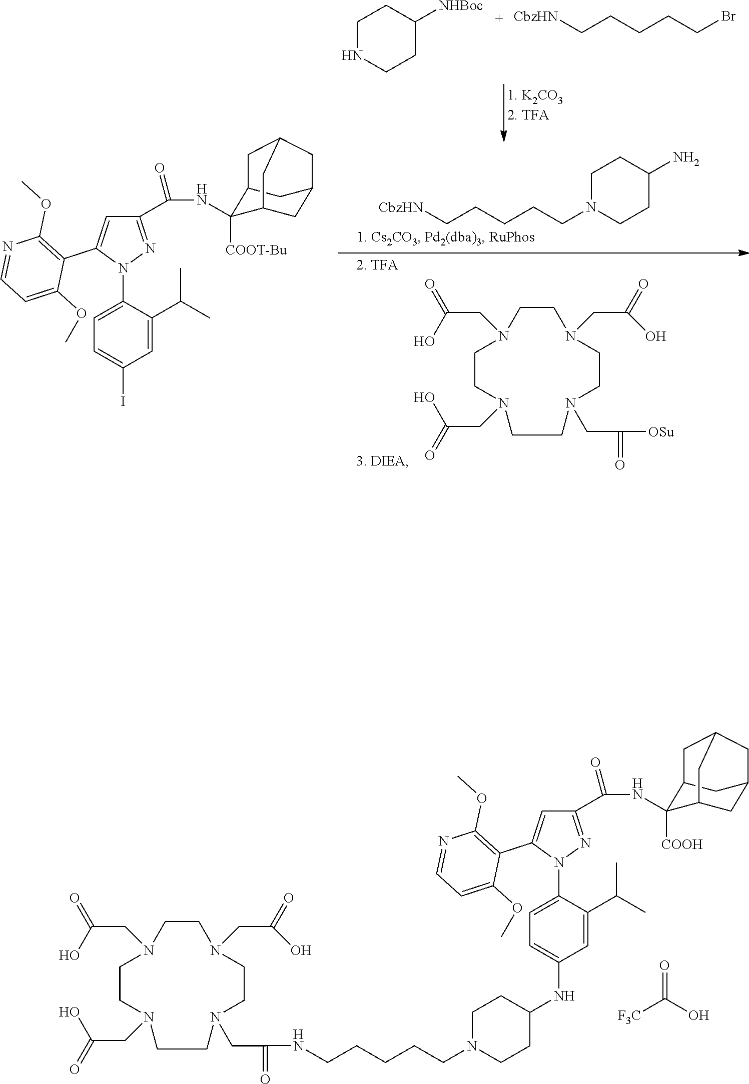

Steps 1-2: tert-Butyl piperidin-4-ylcarbamate (310 mg, 1 Eq, 1.55 mmol) and benzyl (5-bromopentyl)carbamate (697 μg, 1.50 Eq, 2.32 mmol) were treated with K₂CO₃ in a manner similar to Example 58, Step 2 then treated with TFA at room temperature for 1 hour in a manner similar to Example 13, Step 4 to provide (1R,3S,5r,7r)-2-(1-(4-((1-(5-aminopentyl)piperidin-4-yl)amino)-2-isopropylphenyl)-5-(2,4-dimethoxypyridin-3-yl)-1H-pyrazole-3-carboxamido)adamantane-2-carboxylic acid (40 mg, 55 μmol, 97%) which was directly used in the next step without any purification. [M+H]⁺=320.1.

Step 3: (1R,3S,5r,7r)-2-(1-(4-((1-(5-aminopentyl)piperidin-4-yl)amino)-2-isopropylphenyl)-5-(2,4-dimethoxypyridin-3-yl)-1H-pyrazole-3-carboxamido)adamantane-2-carboxylic acid was treated with Pd₂(dba)₃, RuPhos, and Cs₂CO₃ in a manner similar to Step 1 in the synthesis of Compound 86 to provide tert-butyl (1R,3S,5r,7r)-2-(1-(4-((1-(5-(((benzyloxy)carbonyl)amino)pentyl)piperidin-4-yl)amino)-2-isopropylphenyl)-5-(2,4-dimethoxypyridin-3-yl)-1H-pyrazole-3-carboxamido)adamantane-2-carboxylate (49 mg, 53 μmol, 39%) as a yellow oil. [M+H]⁺=918.8.

Steps 4-5: tert-Butyl (1R,3S,5r,7r)-2-(1-(4-((1-(5-(((benzyloxy)carbonyl)amino)pentyl)piperidin-4-yl)amino)-2-isopropylphenyl)-5-(2,4-dimethoxypyridin-3-yl)-1H-pyrazole-3-carboxamido)adamantane-2-carboxylate was treated with TFA in a manner similar to Example 104, Step 2 then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((5-(4-((4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)amino)piperidin-1-yl)pentyl)amino)-2-oxoethyl)-1,4,7,10-tetraazacyclododecane-1,4,7-triyl)triacetic acid (24.3 mg, 19.8 μmol, 36%) as a white solid. MS: Calc'd for C₅₇H₈₃N₁₀O₁₂: 1113.62, found [M+H]⁺: 1114.5.

### Example 123: Compound 123

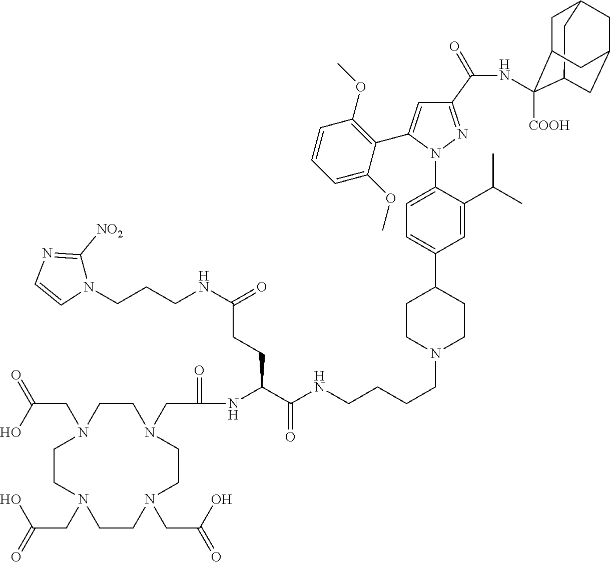 [SMILES: COc1cccc(OC)c1-c1cc(C(=O)NC2(C(=O)O)C3CC4CC(C3)CC2C4)nn1-c1ccc(C2CCN(CCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)CC2)cc1C(C)C]

Step 1: 2,2′,2″-(10-(2-(((2S)-1-((4-(6-(3-(((1R,2s,3S,5r)-2-carboxyadamantan-2-yl)carbamoyl)-5-(2,6-dimethoxyphenyl)-1H-pyrazol-1-yl)-7-isopropyl-3,4-dihydroisoquinolin-2(1H)-yl)butyl)amino)-5-((3-(2-nitro-1H-imidazol-1-yl)propyl)amino)-1,5-dioxopentan-2-yl)amino)-2-oxoethyl)-1,4,7,10-tetraazacyclododecane-1,4,7-triyl)triacetic acid was synthesized from tert-butyl (1R,3 S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate (100 mg, 1 Eq, 153 kmol) in a manner similar to Example 106 to provide the 2,2,2-trifluoroacetic acid salt of the product (7.3 mg, 4.8 μmol, 19%) as a white solid. MS: Calc'd for C₆₆H₉₂N₁₄O₁₆: 1336.68, found [M+K]⁺: 1375.9.

### Example 124: Compound 124

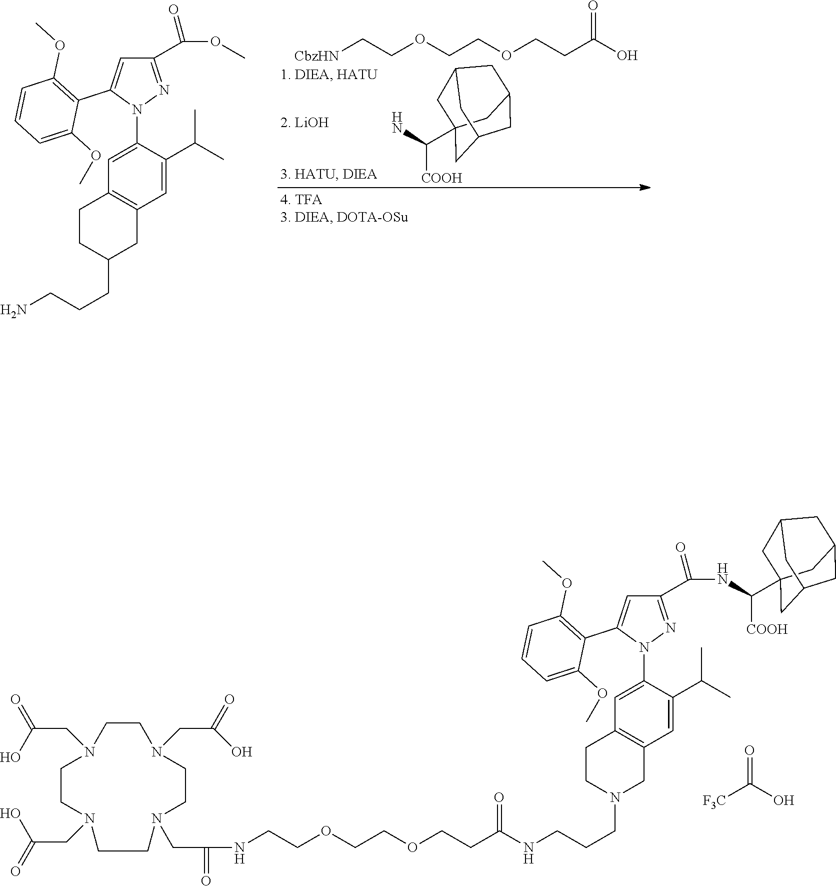

Step 1: Methyl 1-(2-(3-aminopropyl)-7-isopropyl-1,2,3,4-tetrahydroisoquinolin-6-yl)-5-(2,6-dimethoxyphenyl)-1H-pyrazole-3-carboxylate (160 mg, 1 Eq, 325 kmol) and 3-oxo-1-phenyl-2,7,10-trioxa-4-azatidecan-13-oic acid (106 mg, 1.05 Eq, 340 mol) were treated with HATU and DIEA in a manner similar to Example 104, Step 1 to provide methyl 5-(2,6-dimethoxyphenyl)-1-(2-(3,13-dioxo-1-phenyl-2,7,10-trioxa-4,14-diazaheptadecan-17-yl)-7-isopropyl-1,2,3,4-tetrahydroisoquinolin-6-yl)-1H-pyrazole-3-carboxylate (110 μg, 140 mol, 43.1%) as a white solid. [M+H]⁺=786.3.

Steps 2-3: Methyl 5-(2,6-dimethoxyphenyl)-1-(2-(3,13-dioxo-1-phenyl-2,7,10-trioxa-4,14-diazaheptadecan-17-yl)-7-isopropyl-1,2,3,4-tetrahydroisoquinolin-6-yl)-1H-pyrazole-3-carboxylate was treated with LiOH in a manner similar to Example 1, Step 4 then treated with (2S)-2-((3R,5R)-adamantan-1-yl)-2-aminoacetic acid in a manner similar to Example 103, Step 3 to provide (2S)-2-((3R,5R)-adamantan-1-yl)-2-(5-(2,6-dimethoxyphenyl)-1-(2-(3,13-dioxo-1-phenyl-2,7,10-trioxa-4,14-diazaheptadecan-17-yl)-7-isopropyl-1,2,3,4-tetrahydroisoquinolin-6-yl)-1H-pyrazole-3-carboxamido)acetic acid (80 mg, 83 μmol, 80%) as a white solid. [M+H]⁺=963.4.

Steps 4-5: (2S)-2-((3R,5R)-adamantan-1-yl)-2-(5-(2,6-dimethoxyphenyl)-1-(2-(3,13-dioxo-1-phenyl-2,7,10-trioxa-4,14-diazaheptadecan-17-yl)-7-isopropyl-1,2,3,4-tetrahydroisoquinolin-6-yl)-1H-pyrazole-3-carboxamido)acetic acid was treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(16-(6-(3-(((1 S)-((3R,5R)-adamantan-1-yl)(carboxy)methyl)carbamoyl)-5-(2,6-dimethoxyphenyl)-1H-pyrazol-1-yl)-7-isopropyl-3,4-dihydroisoquinolin-2(1H)-yl)-2,12-dioxo-6,9-dioxa-3,13-diazahexadecyl)-1,4,7,10-tetraazacyclododecane-1,4,7-triyl)triacetic acid (32.1 mg, 24.1 μmol, 50%) as a white solid. MS: Calc'd for C₆₂H₉₀N₁₀O₁₅: 1214.66, found [M+H]⁺: 1215.6.

### Example 125: Compound 125

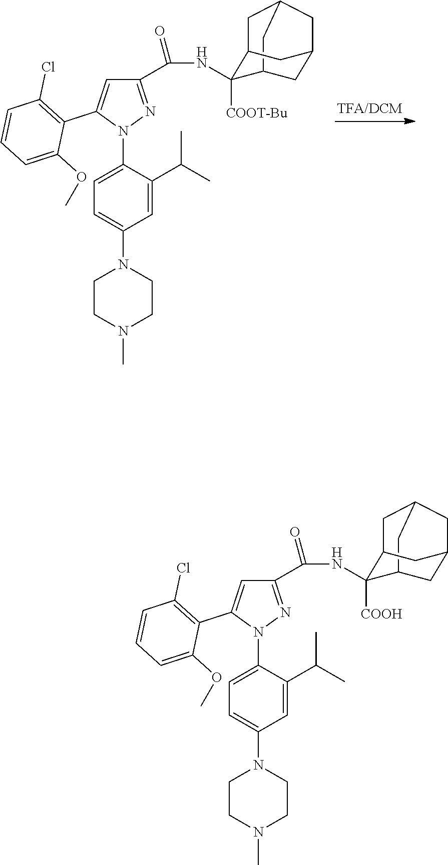

To a mixture of tert-butyl (1R,3S,5r,7r)-2-(5-(2-chloro-6-methoxyphenyl)-1-(2-isopropyl-4-(4-methylpiperazin-1-yl)phenyl)-1H-pyrazole-3-carboxamido)adamantane-2-carboxylate (20 mg, 1 Eq, 28 μmol) (from Example 80, Step 4) and DCM (0.5 mL) was added TFA (0.1 mL). The reaction mixture was stirred at 25° C. for 1 hour. The crude product was purified by Prep-HPLC to provide the 2,2,2-trifluoroacetic acid salt of (1R,3S,5r,7r)-2-(5-(2-chloro-6-methoxyphenyl)-1-(2-isopropyl-4-(4-methylpiperazin-1-yl)phenyl)-1H-pyrazole-3-carboxamido)adamantane-2-carboxylic acid (4.5 mg, 5.9 μmol, 21%) as a white solid. MS: Calc'd for C₃₆H₄₄ClN₅O₄: 645.31, found [M+H]⁺: 646.4, 648.4.

### Example 126: Compound 126

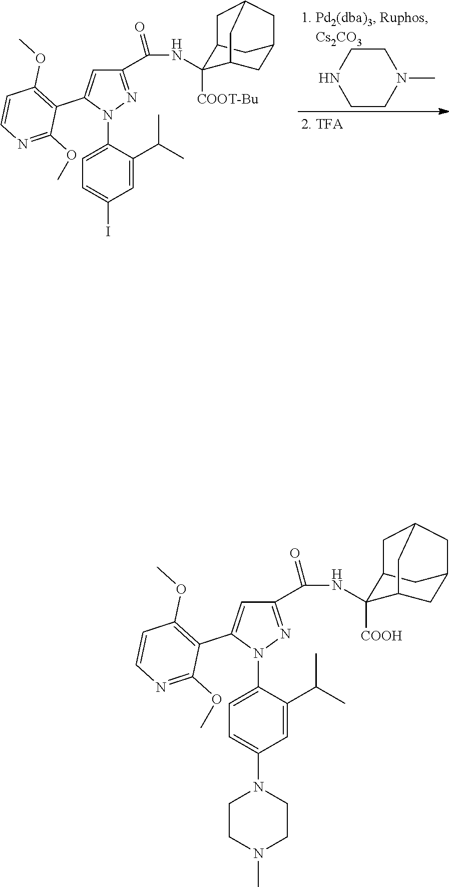

tert-Butyl (1R,3S,5r,7r)-2-(5-(2,4-dimethoxypyridin-3-yl)-1-(4-iodo-2-isopropylphenyl)-1H-pyrazole-3-carboxamido)adamantane-2-carboxylate (30 mg, 1 Eq, 41 μmol) (Example 83, Step 6) was treated with 1-methylpiperazine in a manner similar to Step 1 in the Synthesis of Compound 86 then treated with TFA in a manner similar to Example 13, Steps 4 to provide 2,2,2-trifluoroacetic acid salt of (1R,3S,5r,7r)-2-(5-(2,4-dimethoxypyridin-3-yl)-1-(2-isopropyl-4-(4-methylpiperazin-1-yl)phenyl)-1H-pyrazole-3-carboxamido)adamantane-2-carboxylic acid (14.1 mg, 18.6 μmol, 52%) as a white solid. MS: Calc'd for C₃₆H₄₆N₆O₆: 642.35, found [M+H]⁺: 643.3.

### Example 127: Compound 127

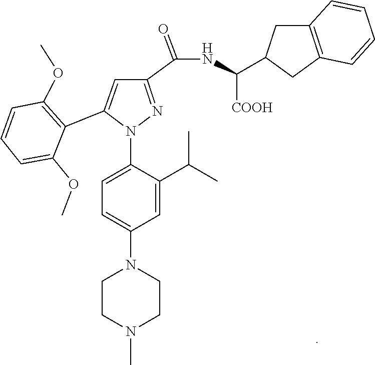 [SMILES: COc1cccc(OC)c1-c1cc(C(=O)N[C@H](C(=O)O)C2Cc3ccccc3C2)nn1-c1ccc(N2CCN(C)CC2)cc1C(C)C]

A mixture of 5-(2,6-dimethoxyphenyl)-1-(2-isopropyl-4-(4-methylpiperazin-1-yl)phenyl)-1H-pyrazole-3-carboxylic acid (20 mg, 1 Eq, 43 μmol) (from Example 76, Step 2), HATU (25 mg, 1.5 Eq, 66 μmol), DIEA (17 mg, 23 μL, 3.1 Eq, 0.13 mmol) and DMF (2 mL) was stirred at 25° C. for 10 minutes, then (S)-2-amino-2-(2,3-dihydro-1H-inden-2-yl)acetic acid (10 mug, 1.2 Eq, 52 μmol) was added and the reaction mixture was stirred at 25° C. for an additional 1 hour. The crude product was purified by Prep-HPLC to provide the 2,2,2-trifluoroacetic acid salt of(S)-2-(2,3-dihydro-1H-inden-2-yl)-2-(5-(2,6-dimethoxyphenyl)-1-(2-isopropyl-4-(4-methylpiperazin-1-yl)phenyl)-1H-pyrazole-3-carboxamido)acetic acid (9.7 mg, 13 mol, 30%) as a white solid. MS: Calc'd for C₃₇H₄₃N₅O₅: 637.3, found [M+H]⁺: 638.3.

### Example 128: Compound 128

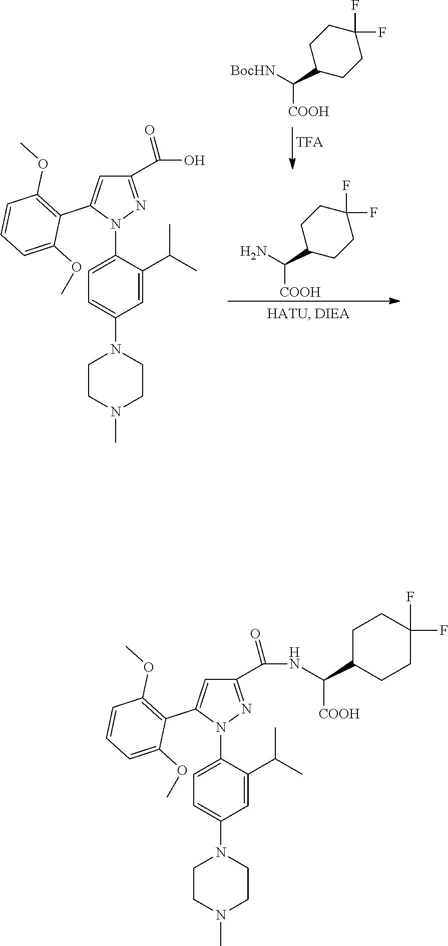 [SMILES: CC(C)(C)OC(=O)N[C@H](C(=O)O)C1CCC(F)(F)CC1 ; COc1cccc(OC)c1-c1cc(C(=O)O)nn1-c1ccc(N2CCN(C)CC2)cc1C(C)C ; N[C@H](C(=O)O)C1CCC(F)(F)CC1 ; COc1cccc(OC)c1-c1cc(C(=O)N[C@H](C(=O)O)C2CCC(F)(F)CC2)nn1-c1ccc(N2CCN(C)CC2)cc1C(C)C]

Step 1: To a mixture of (S)-2-((tert-butoxycarbonyl)amino)-2-(4,4-difluorocyclohexyl)acetic acid (30 mg, 1 Eq, 0.10 mmol) and DCM (1.5 mL), was added TFA (0.3 mL) then the reaction mixture was stirred at 25° C. for 30 min. The mixture was concentrated under reduced pressure then purified by Prep-HPLC to provide (S)-2-amino-2-(4,4-difluorocyclohexyl)acetic acid (15 mg, 78 mol, 76%) as a white solid.

Step 2: 5-(2,6-Dimethoxyphenyl)-1-(2-isopropyl-4-(4-methylpiperazin-1-yl)phenyl)-1H-pyrazole-3-carboxylic acid (20 mg, 1 Eq, 43 μmol) (from Example 76, Step 2), was treated with (S)-2-amino-2-(4,4-difluorocyclohexyl)acetic acid, HATU, and DIEA in a manner similar to Example 127 to provide the 2,2,2-trifluoroacetic acid salt of (S)-2-(4,4-difluorocyclohexyl)-2-(5-(2,6-dimethoxyphenyl)-1-(2-isopropyl-4-(4-methylpiperazin-1-yl)phenyl)-1H-pyrazole-3-carboxamido)acetic acid (10 mg, 13 μmol, 26%) as a white solid. MS: Calc'd for C₃₄H₄₃F₂N₅O₅: 639.32, found [M+H]⁺: 640.3.

### Example 129: Compound 129

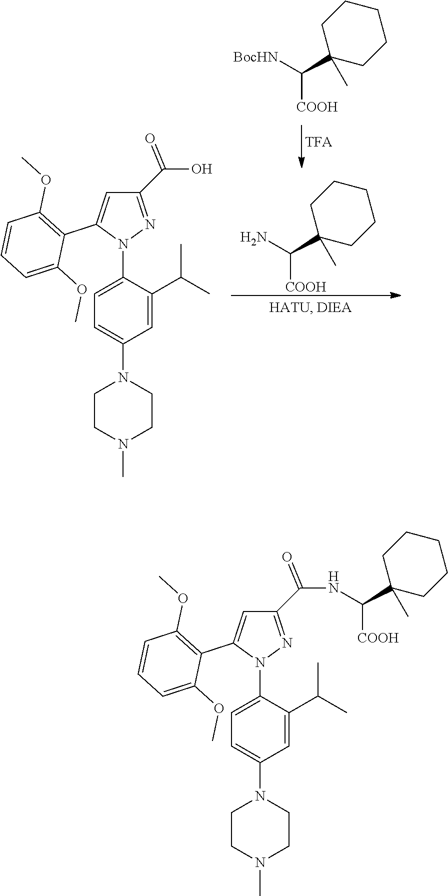 [SMILES: CC(C)(C)OC(=O)N[C@H](C(=O)O)C1(C)CCCCC1 ; COc1cccc(OC)c1-c1cc(C(=O)O)nn1-c1ccc(N2CCN(C)CC2)cc1C(C)C ; CC1([C@H](N)C(=O)O)CCCCC1 ; COc1cccc(OC)c1-c1cc(C(=O)N[C@H](C(=O)O)C2(C)CCCCC2)nn1-c1ccc(N2CCN(C)CC2)cc1C(C)C]

Step 1: To a mixture of (S)-2-((tert-butoxycarbonyl)amino)-2-(1-methylcyclohexyl)acetic acid (30 mg, 1 Eq, 0.11 mmol) and DCM (1.5 mL) was added TFA (0.3 mL). The reaction mixture was stirred at 25° C. for 30 min. then the crude product was purified by Prep-HPLC to afford (S)-2-amino-2-(1-methylcyclohexyl)acetic acid (10 mg, 58 μmol, 53%) as a white solid.

Step 2: 5-(2,6-Dimethoxyphenyl)-1-(2-isopropyl-4-(4-methylpiperazin-1-yl)phenyl)-1H-pyrazole-3-carboxylic acid (20 mg, 1 Eq, 43 mol) (from Example 76, Step 2), was treated with (S)-2-amino-2-(1-methylcyclohexyl)acetic acid, HATU, and DIEA in a manner similar to Example 127 to provide the 2,2,2-trifluoroacetic acid salt of (S)-2-(5-(2,6-dimethoxyphenyl)-1-(2-isopropyl-4-(4-methylpiperazin-1-yl)phenyl)-1H-pyrazole-3-carboxamido)-2-(1-methylcyclohexyl)acetic acid (15 mg, 20 μmol, 35%) as a white solid. MS: Calc'd for C₃₅H₄₇N₅O₅: 617.36, found [M+H]⁺: 618.3.

### Example 130: Compound 130

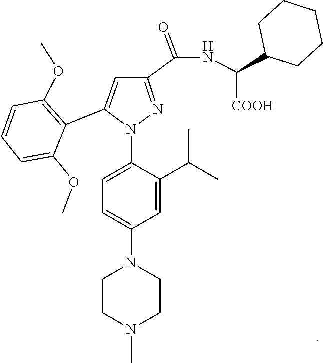 [SMILES: COc1cccc(OC)c1-c1cc(C(=O)N[C@H](C(=O)O)C2CCCCC2)nn1-c1ccc(N2CCN(C)CC2)cc1C(C)C]

5-(2,6-Dimethoxyphenyl)-1-(2-isopropyl-4-(4-methylpiperazin-1-yl)phenyl)-1H-pyrazole-3-carboxylic acid (20 mg, 1 Eq, 43 μmol) (from Example 76, Step 2), was treated with (S)-2-amino-2-cyclohexylacetic acid, HATU, and DIEA in a manner similar to Example 127 to provide the 2,2,2-trifluoroacetic acid salt of (S)-2-cyclohexyl-2-(5-(2,6-dimethoxyphenyl)-1-(2-isopropyl-4-(4-methylpiperazin-1-yl)phenyl)-1H-pyrazole-3-carboxamido)acetic acid (4.8 mg, 6.7 μmol, 21%) as a white solid. MS: Calc'd for C₃₄H₁₅N₅O₅: 603.34, found [M+H]⁺: 604.4.

### Example 131: Compound 131

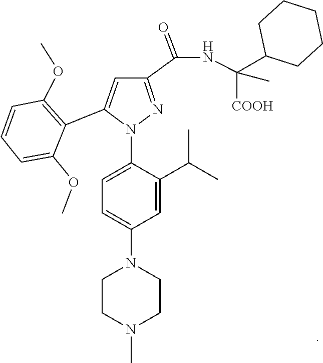 [SMILES: COc1cccc(OC)c1-c1cc(C(=O)NC(C)(C(=O)O)C2CCCCC2)nn1-c1ccc(N2CCN(C)CC2)cc1C(C)C]

5-(2,6-Dimethoxyphenyl)-1-(2-isopropyl-4-(4-methylpiperazin-1-yl)phenyl)-1H-pyrazole-3-carboxylic acid (20 mg, 1 Eq, 43 μmol) (from Example 76, Step 2), was treated with 2-amino-2-cyclohexylpropanoic acid, HA TU, and DIEA in a manner similar to Example 127 to provide the 2,2,2-trifluoroacetic acid salt of 2-cyclohexyl-2-(5-(2,6-dimethoxyphenyl)-1-(2-isopropyl-4-(4-methylpiperazin-1-yl)phenyl)-1H-pyrazole-3-carboxamido)propanoic acid (5.0 mg, 6.8 μmol, 21%) as a white solid. MS: Calc'd for C₃₅H₄₇N₅O₅: 617.36, found [M+H]⁺: 618.4.

### Example 132: Compound 132

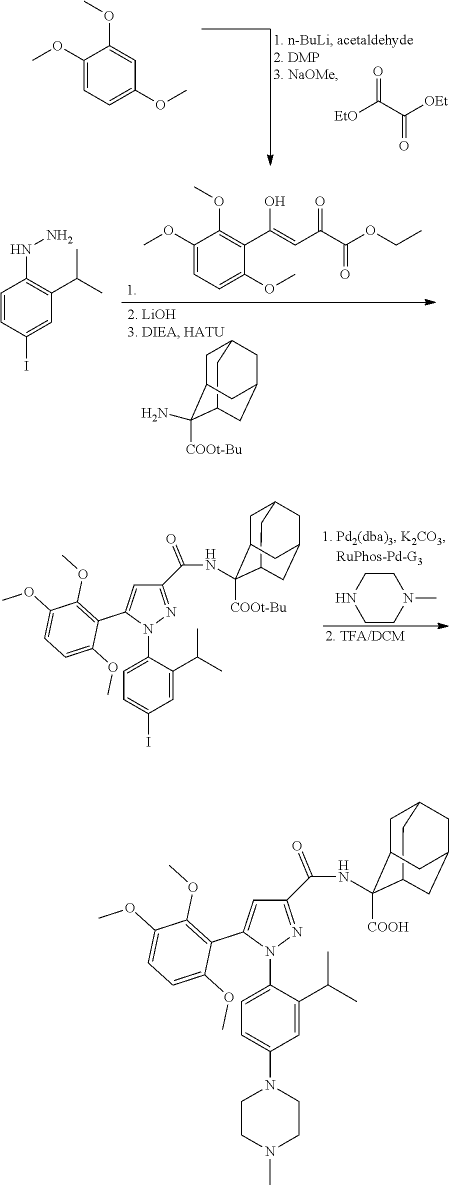

Steps 1-6: tert-Butyl (1R,3S,5r,7r)-2-(1-(4-iodo-2-isopropylphenyl)-5-(2,3,6-trimethoxyphenyl)-1H-pyrazole-3-carboxamido)adamantane-2-carboxylate was synthesized from 1,2,4-trimethoxybenzene (3.0 g, 1 Eq, 18 mmol) in a manner similar to Example 83, Steps 1-6 to afford the product (80 mg, 0.11 mmol, 61%) as a white solid.

Step 7: A mixture of tert-butyl (1R,3 S,5r,7r)-2-(1-(4-iodo-2-isopropylphenyl)-5-(2,3,6-trimethoxyphenyl)-1H-pyrazole-3-carboxamido)adamantane-2-carboxylate (25 mg, 1 Eq, 33 μmol), (2-dicyclohexylphosphino-2′,6′-diisopropoxy-1,1′-biphenyl)[2-(2′-amino-1,1′-biphenyl)]palladium(II) methanesulfonate (2.8 mg, 0.10 Eq, 3.3 μmol), 1-methylpiperazine (5.0 mg, 1.5 Eq, 50 μmol), K₂CO₃ (14 mg, 3.1 Eq, 0.10 mmol), Pd₂(dba)₃ (3.0 mg, 0.099 Eq, 3.3 μmol) and 1,4-dioxane (2 mL) under N₂ was stirred for 100° C. at 2 hour. The mixture was concentrated and the crude product was purified by MPLC to afford tert-butyl (1R,3S,5r,7r)-2-(1-(2-isopropyl-4-(4-methylpiperazin-1-yl)phenyl)-5-(2,3,6-trimethoxyphenyl)-1H-pyrazole-3-carboxamido)adamantane-2-carboxylate (9.6 mg, 13 μmol, 40%) as a white solid.

Step 8: tert-Butyl (1R,3S,5r,7r)-2-(1-(2-isopropyl-4-(4-methylpiperazin-1-yl)phenyl)-5-(2,3,6-trimethoxyphenyl)-1H-pyrazole-3-carboxamido)adamantane-2-carboxylate was treated with TFA in a manner similar to Example 1, Step 8 to provide the 2,2,2-trifluoroacetic acid salt of (1R,3S,5r,7r)-2-(1-(2-isopropyl-4-(4-methylpiperazin-1-yl)phenyl)-5-(2,3,6-trimethoxyphenyl)-1H-pyrazole-3-carboxamido)adamantane-2-carboxylic acid (3 mg, 4 μmol, 30%) as a white solid. MS: Calc'd for C₃₈H₄₉N₅O₆: 671.37, found [M+H]⁺: 672.3.

### Example 133: Compound 133

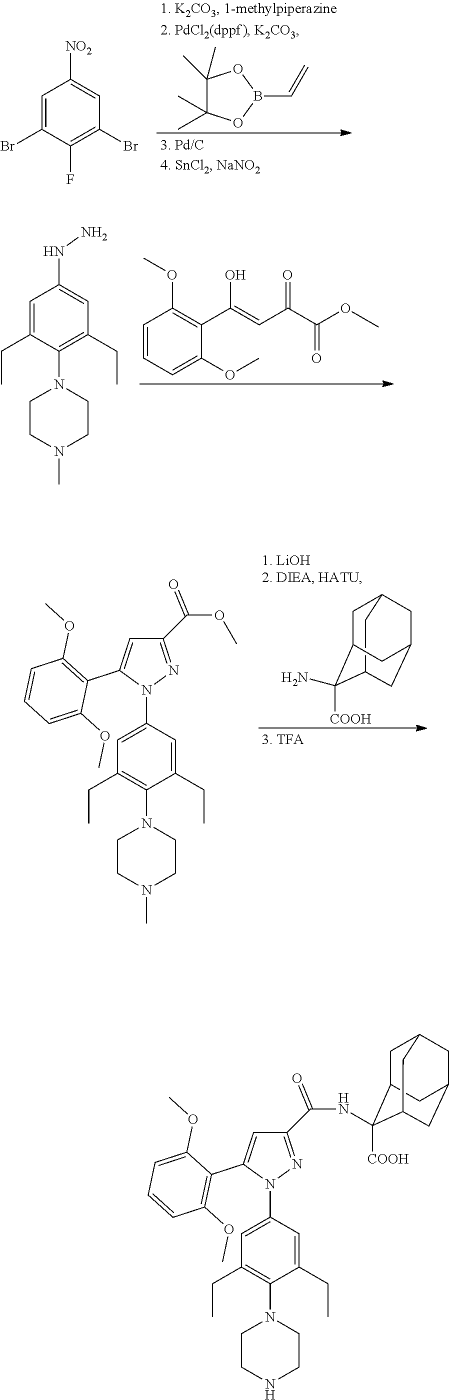

To a mixture of 1,3-dibromo-2-fluoro-5-nitrobenzene (5 g, 1 Eq, 0.02 mol) and potassium carbonate (7 g, 3 Eq, 0.05 mol) was added ACN (50 mL) and 1-methylpiperazine (5 & 3 Eq, 0.05 mol). The reaction mixture was stirred at 100° C. for 3 hours. The mixture was concentrated under reduced pressure. The crude product was purified by MPLC to afford 1-(2,6-dibromo-4-nitrophenyl)-4-methylpiperazine (4.5 g, 12 mmol, 70%) as a yellow crude oil.

Step 2: To a mixture of 1-(2,6-dibromo-4-nitrophenyl)-4-methylpiperazine (4.5 g, 1 Eq, 12 mmol), 4,4,5,5-tetramethyl-2-vinyl-1,3,2-dioxaborolane (2.2 g, 1.2 Eq, 14 mmol), K₂CO₃ (4.9 g, 3.0 Eq, 35 mmol), 1,4-dioxane (40 mL) and 1120 (10 mL), was added PdCl₂(dppf) (0.87 g, 0.10 Eq, 1.2 mmol) under N₂. The reaction mixture was stirred at 90° C. for 1 hour. The crude product was isolated by filtration to provide 1-methyl-4-(4-nitro-2,6-divinylphenyl)piperazine (3.0 g, 11 mmol, 92%) as a white crude solid, which was used directly in the next step without any purification.

Step 3: To a mixture of 1-methyl-4-(4-nitro-2,6-divinylphenyl)piperazine (3.0 g, 1 Eq, 11 mmol) and MeOH (50 mL) under N₂, was carefully added Pd—C (1.2 g, 10% Wt, 0.10 Eq, 1.1 mmol). The flask was evacuated and flushed with hydrogen three times. The mixture was stirred for 25° C. for 1 h under H₂. The reaction mixture was filtered through a pad of celite, then the filtrate was concentrated and purified by MPLC to afford 3,5-diethyl-4-(4-methylpiperazin-1-yl)aniline (1.6 g, 6.5 mmol, 59%) as a white solid.

Step 4: To a mixture of 3,5-diethyl-4-(4-methylpiperazin-1-yl)aniline (210 mg, 1 Eq, 849 μmol) and HCl (10 mL) under N₂ was added sodium nitrite (293 mg, 177 μL, 5.00 Eq, 4.25 mmol) dropwise at −78° C. over 10 min. The reaction mixture was stirred at 0° C. for 3 h then dichlorostannane (193 mg, 50.0 μL, 1.20 Eq, 1.02 mmol) was added dropwise at −78° C. over 10 min. The reaction mixture was stirred at 0° C. for 2 hours. The reaction was cooled to room temperature, diluted with PE, then the crude solid was collected by filtration to afford 1-(2,6-diethyl-4-hydrazineylphenyl)-4-methylpiperazine (165 mg, 629 mol, 74.1%) as a yellow solid.

Step 5: To a mixture of 1-(2,6-diethyl-4-hydrazineylphenyl)-4-methylpiperazine (165 mug, 1 Eq, 629 μmol) and methyl (Z)-4-(2,6-dimethoxyphenyl)-4-hydroxy-2-oxobut-3-enoate (201 mg, 1.20 Eq, 755 μmol) was added AcOH (10 mL). The reaction mixture was stirred at 90° C. for 1 hour, then the mixture was concentrated under reduced pressure to afford methyl 1-(3,5-diethyl-4-(4-methylpiperazin-1-yl)phenyl)-5-(2,6-dimethoxyphenyl)-1H-pyrazole-3-carboxylate (45 mg, 91 μmol, 15%) as a yellow solid, which was used directly in the next step without further purification.

Steps 6-8: (1R,3S,5r,7r)-2-(1-(3,5-diethyl-4-(4-methylpiperazin-1-yl)phenyl)-5-(2,6-dimethoxyphenyl)-1H-pyrazole-3-carboxamido)adamantane-2-carboxylic acid was synthesized from methyl 1-(3,5-diethyl-4-(4-methylpiperazin-1-yl)phenyl)-5-(2,6-dimethoxyphenyl)-1H-pyrazole-3-carboxylate (45 mg, 1 Eq, 91 μmol) in a manner similar to Example 9, Steps 6-8, to provide the 2,2,2-trifluoracetic acid salt of the product (5 mg, 6 μmol, 20%) as a white solid. [M+H]⁺=656.5.

MS: Calc'd for C₃₈H₄₉N₅O₅: 655.37, found [M+H]⁺: 656.5.

### Example 134: Compound 134

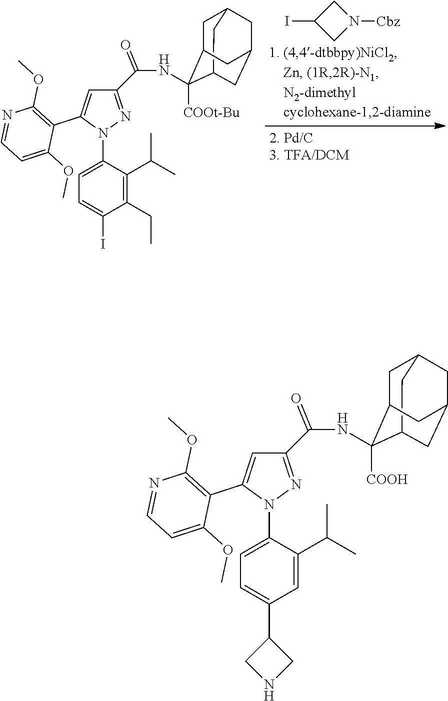

Step 1: A mixture of tert-butyl (1R,3S,5r,7r)-2-(5-(2,4-dimethoxypyridin-3-yl)-1-(4-iodo-2-isopropylphenyl)-1H-pyrazole-3-carboxamido)adamantane-2-carboxylate (from Compound 83, Step 6) (470 mg, 1 Eq, 647 μmol), [4,4′-bis(1,1-dimethylethyl)-2,2′-bipyridine]nickel (II) dichloride (64.4 mg, 0.250Eq, 162 μmol), (1R,2R)—N1,N2-dimethylcyclohexane-1,2-diamine (46.0 mg, 0.500Eq, 323 μmol), benzyl 3-iodoazetidine-1-carboxylate (410 mg, 2.00 Eq, 1.29 mmol) and DMAc (6 mL) was stirred at 60° C. for 16 hours, then zinc (148 mg, 3.50 Eq, 2.26 mmol) was added and the reaction mixture was stirred at 60° C. for an additional 1 hour. The mixture was directly purified by MPLC to afford benzyl 3-(4-(3-(((1R,3S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)azetidine-1-carboxylate (170 mg, 215 μmol, 33.3%) as a yellow oil. [M+H]⁺=790.4.

Step 2: A mixture of benzyl 3-(4-(3-(((1R,3 S,5r,7r)-2-(tert-butoxycarbonyl)adamantan-2-yl)carbamoyl)-5-(2,4-dimethoxypyridin-3-yl)-1H-pyrazol-1-yl)-3-isopropylphenyl)azetidine-1-carboxylate (170 mg, 1 Eq, 215 μmol) and MeOH (5 mL) under N₂ was carefully added Pd/C (68 mg, 10% Wt, 0.30 Eq, 64 μmol). The flask was evacuated and flushed with hydrogen three times. The mixture was stirred at 45° C. for 2 h under H₂. The reaction mixture was filtered through a pad of celite, then the filtrate was concentrated to afford tert-butyl (1R,3 S,5r,7r)-2-(1-(4-(azetidin-3-yl)-2-isopropylphenyl)-5-(2,4-dimethoxypyridin-3-yl)-1H-pyrazole-3-carboxamido)adamantane-2-carboxylate (120 mg, 183 μmol, 85.0%) as a yellow oil. [M+H]⁺=656.4.

Step 3: The product from Step 2 was treated with TFA in a manner similar to Example 1, Step 8 to provide the 2,2,2-trifluoroacetic acid salt of (1R,3S,5r,7r)-2-(1-(4-(azetidin-3-yl)-2-isopropylphenyl)-5-(2,4-dimethoxypyridin-3-yl)-1H-pyrazole-3-carboxamido)adamantane-2-carboxylic acid (5.4 mg, 7.6 μmol, 50%) as a white solid. MS: Calc'd for C₃₄H₄₁N₅O₅ 599.31, found [M+H]⁺: 600.6.

### Example 135: Compound 135A and Compound 135B

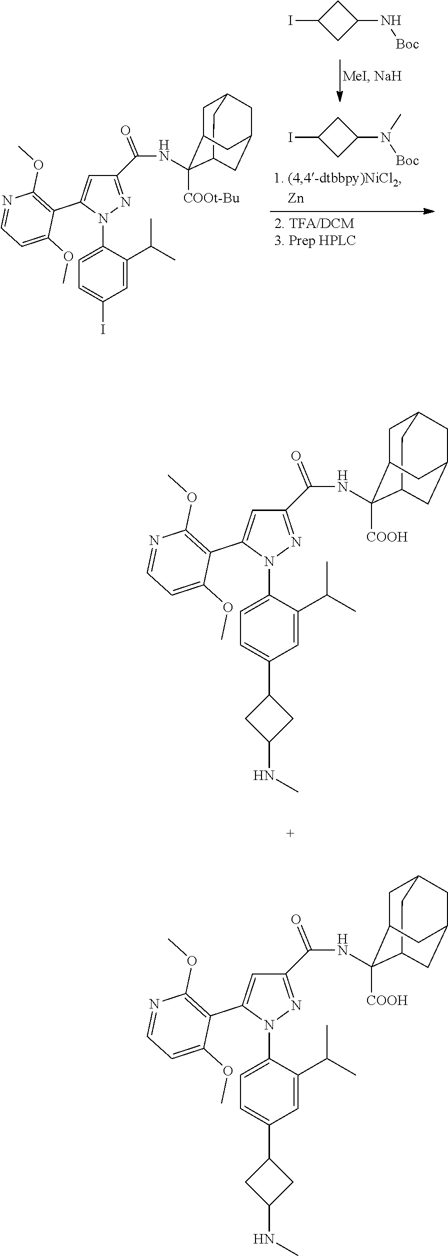

Step 1: A mixture of tert-butyl (3-iodocyclobutyl)carbamate (110 mg, 1 Eq, 370 μmol), NaH (13.3 mg, 1.50 Eq, 554 μmol) and DMF (5 mL) was stirred at 0° C. for 10 minutes, then methyl iodide (105 mg, 47.9 μL, 2.00 Eq, 740 μmol) was added and the reaction mixture was stirred at 25° C. for an additional 1 hour. The mixture was directly purified by MPLC to afford tert-butyl (3-iodocyclobutyl)(methyl)carbamate (70 mg, 0.22 mmol, 61%) as an off-yellow solid. [M-t-Bu+H]⁺=253.0, [M−Boc+H]=212.0.

Step 2: A mixture of tert-butyl (1R,3S,5r,7r)-2-(5-(2,4-dimethoxypyridin-3-yl)-1-(4-iodo-2-isopropylphenyl)-1H-pyrazole-3-carboxamido)adamantane-2-carboxylate (60 mg, 1 Eq, 83 μmol), [4,4′-bis(1,1-dimethylethyl)-2,2′-bipyridine]nickel (II) dichloride (7.5 mg, 0.20 Eq, 16 μmol), tert-butyl (3-iodocyclobutyl)(methyl)carbamate (51 mg, 2.0 Eq, 0.16 mmol) and DME (3 mL) was stirred at 60° C. for 5 h, then Zn (54 mg, 10 Eq, 0.83 mmol) was added and the reaction mixture was stirred at 60° C. for an additional 16 hours. The reaction mixture was filtered and the filtrate was collected to provide the crude product, which was purified by MPLC to afford tert-butyl (1R,3S,5r,7r)-2-(1-(4-(3-((tert-butoxycarbonyl)(methyl)amino)cyclobutyl)-2-isopropylphenyl)-5-(2,4-dimethoxypyridin-3-yl)-1H-pyrazole-3-carboxamido)adamantane-2-carboxylate (35 mg, 45 μmol, 54%) as a yellow solid. [M+H]⁺=784.4.

Step 3A: The product from Step 2 was treated with TFA in a manner similar to Example 1, Step 8, then purified by prep-HPLC to provide the 2,2,2-trifluoroacetic acid salt of a single isomer of (1R,3S,5R,7R)-2-(5-(2,4-dimethoxypyridin-3-yl)-1-(2-isopropyl-4-((1s,3s)-3-(methylamino)cyclobutyl)phenyl)-1H-pyrazole-3-carboxamido)adamantane-2-carboxylic acid (5.6 mg, 7.5 μmol, 17%) as a white solid. MS: Calc'd for C₃₆H₄₅N₅O₅: 627.34, found [M+H]⁺: 628.4.

Step 3B: The 2^{nd} isomer of (1R,3 S,5R,7R)-2-(5-(2,4-dimethoxypyridin-3-yl)-1-(2-isopropyl-4-((1r,3r)-3-(methylamino)cyclobutyl)phenyl)-1H-pyrazole-3-carboxamido)adamantane-2-carboxylic acid was isolated from the prep HPLC separation as a 2,2,2-trifluoroacetic acid salt (4.7 mg, 6.3 μmol, 14%) as a white solid. MS: Calc'd for C₃₆H₄₅N₅O₅: 627.34, found [M+H]⁺: 628.4.

### Example 136: Compound 136

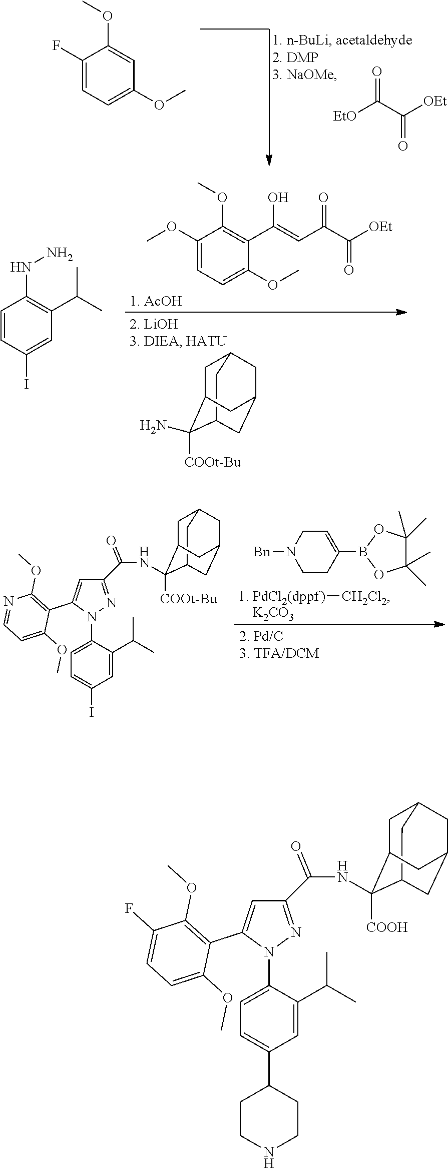

(1R,3S,5r,7r)-2-(5-(3-fluoro-2,6-dimethoxyphenyl)-1-(2-isopropyl-4-(piperidin-4-yl)phenyl)-1H-f-pyrazole-3-carboxamido)adamantane-2-carboxylic acid was synthesized from 1-fluoro-2,4-dimethoxybenzene (2.0 g, 1 Eq, 13 mmol) in a manner similar to the synthesis of Example 83 (Compound 83) to provide the 2,2,2-trifluoroacetic acid of the product (5.5 mg, 7.2 μmol, 25%) as a white solid. MS: Calc'd for C₃₇H₄₅FN₄O₅: 644.34, found [M+H]⁺: 645.4.

### Example 137: Compound 137

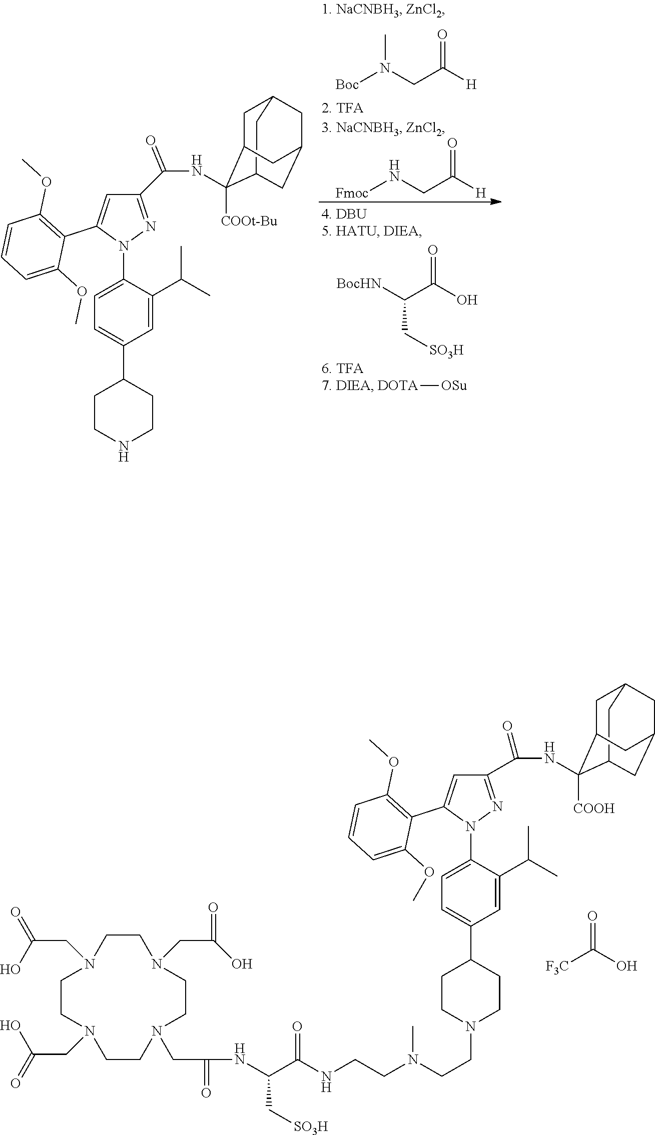

Step 1: A mixture of tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate(160 mg, 1 Eq, 234 μmol), tert-butyl methyl(2-oxoethyl)carbamate (41 mg, 1.0 Eq, 0.24 mmol), ZnCl₂ (64 mg, 2.0 Eq, 0.47 mmol) and MeOH (4 mL) was stirred at 20° C. for 10 minutes, then NaCNBH₃ (30 mg, 2.0 Eq, 0.48 mmol) was added and the reaction mixture was stirred at 25° C. for 1 hour. The mixture was directly purified by MPLC to afford tert-butyl (1R,3S,5r,7r)-2-(1-(4-(1-(2-((tert-butoxycarbonyl)(methyl)amino)ethyl)piperidin-4-yl)-2-isopropylphenyl)-5-(2,6-dimethoxyphenyl)-1H-pyrazole-3-carboxamido)adamantane-2-carboxylate (150 mug, 179 μmol, 76.2%) as a yellow oil. [M+H]⁺=840.7.

Step 2: tert-Butyl (1R,3 S,5r,7r)-2-(1-(4-(1-(2-((tert-butoxycarbonyl)(methyl)amino)ethyl)piperidin-4-yl)-2-isopropylphenyl)-5-(2,6-dimethoxyphenyl)-1H-pyrazole-3-carboxamido)adamantane-2-carboxylate was treated with TFA and stirred at 30° C. for 20 min. The reaction was mixture was adjusted to pH 8.0 by addition of a saturated solution of sodium hydroxide. The mixture was extracted with DCM (10 mL×3), dried over anhydrous Na₂SO₄ and concentrated under reduced pressure to afford tert-butyl (1R,3S,5r,7r)-2-(5-(2,6-dimethoxyphenyl)-1-(2-isopropyl-4-(1-(2-(methylamino)ethyl)piperidin-4-yl)phenyl)-1H-pyrazole-3-carboxamido)adamantane-2-carboxylate (110 mg, 149 μmol, 83.3 Steps 3-4: (1R,3S,5r,7r)-2-(1-(4-(1-(2-((2-aminoethyl)(methyl)amino)ethyl)piperidin-4-yl)-2-isopropylphenyl)-5-(2,6-dimethoxyphenyl)-1H-pyrazole-3-carboxamido)adamantane-2-carboxylic acid was synthesized from tert-butyl (1R,3 S,5r,7r)-2-(5-(2,6-dimethoxyphenyl)-1-(2-isopropyl-4-(1-(2-(methylamino)ethyl)piperidin-4-yl)phenyl)-1H-pyrazole-3-carboxamido)adamantane-2-carboxylate (105 mg, 1 Eq, 142 kmol) and (9H-fluoren-9-yl)methyl (2-oxoethyl)carbamate (40 mg, 1.0 Eq, 0.14 mmol) in a manner similar to Step 1, then treated with DBU in a manner similar to Example 68, Step 5 to provide the product (50 mg, 69 μmol, 110%) as a yellow oil.

Steps 5-7: (1R,3S,5r,7r)-2-(1-(4-(1-(2-((2-aminoethyl)(methyl)amino)ethyl)piperidin-4-yl)-2-isopropylphenyl)-5-(2,6-dimethoxyphenyl)-1H-pyrazole-3-carboxamido)adamantane-2-carboxylic acid was treated with (tert-butoxycarbonyl)(sulfo)-D-alanine in a manner similar to Example 103, Step 5 then treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-(((R)-1-((2-((2-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)ethyl)(methyl)amino)ethyl)amino)-1-oxo-3-sulfopropan-2-yl)amino)-2-oxoethyl)-1,4,7,10-tetraazacyclododecane-1,4,7-triyl)triacetic acid (45.5 mg, 33.0 μmol, 72%) as a white solid. MS: Calc'd for C₆₁H₈₉N₁₁O₁₆S: 1263.62, found [M+H]⁺: 1264.7

### Example 138: Compound 138

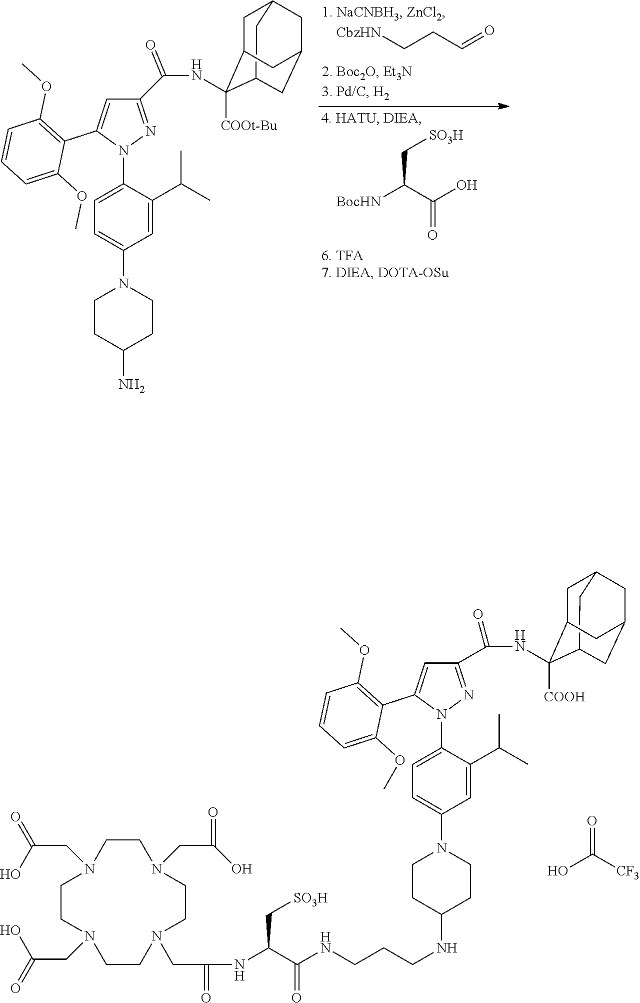

Step 1: A mixture of tert-butyl (1R,3S,5r,7r)-2-(1-(4-(4-aminopiperidin-1-yl)-2-isopropylphenyl)-5-(2,6-dimethoxyphenyl)-1H-pyrazole-3-carboxamido)adamantane-2-carboxylate (180 mg, 1 Eq, 258 μmol), benzyl (3-oxopropyl)carbamate (53.4 mg, 1.0 Eq, 258 μmol), zinc chloride (105 mg, 48.6 μL, 3.0 Eq, 770 μmol) and MeOH (5 mL) was stirred at 25° C. for 10 minutes, then sodium cyanoborohydride (32.4 mg, 30.0 μL, 2.00 Eq, 516 μmol) was added and the reaction mixture was stirred at 25° C. for an additional 1 hour. The mixture was directly purified by MPLC to afford tert-butyl (1R,3S,5r,7r)-2-(1-(4-(4-((3-(((benzyloxy)carbonyl)amino)propyl)amino)piperidin-1-yl)-2-isopropylphenyl)-5-(2,6-dimethoxyphenyl)-1H-pyrazole-3-carboxamido)adamantane-2-carboxylate (60 mg, 67 μmol, 26%) as a white solid. [M+H]⁺:=:889.7.

Step 2: To a mixture of tert-butyl (1R,3S,5r,7r)-2-(1-(4-(4-((3-(((benzyloxy)carbonyl)amino)propyl)amino)piperidin-1-yl)-2-isopropylphenyl)-5-(2,6-dimethoxyphenyl)-1H-pyrazole-3-carboxamido)adamantane-2-carboxylate (60 mg, 1 Eq, 67 μmol), Boc₂O (22 mg, 23 μL, 1.5 Eq, 0.10 mmol) and DCM (2 mL) was added TEA (20 mg, 28 μL, 2.9 Eq, 0.20 mmol). The reaction mixture was stirred at 25° C. for 1 hour. The mixture was concentrated under reduced pressure and the crude product was purified by Prep-HPLC to afford tert-butyl (1R,3S,5r,7r)-2-(1-(4-(4-((3-(((benzyloxy)carbonyl)amino)propyl)(tert-butoxycarbonyl)amino)piperidin-1-yl)-2-isopropylphenyl)-5-(2,6-dimethoxyphenyl)-1H-pyrazole-3-carboxamido)adamantane-2-carboxylate (25 mg, 25 μmol, 37%) as a white solid. [M+H]⁺=989.7.

Step 3: tert-Butyl (1R,3S,5r,7r)-2-(1-(4-(4-((3-(((benzyloxy)carbonyl)amino)propyl)(tert-butoxycarbonyl)amino)piperidin-1-yl)-2-isopropylphenyl)-5-(2,6-dimethoxyphenyl)-1H-pyrazole-3-carboxamido)adamantane-2-carboxylate (25 mg, 1 Eq, 25 μmol) was treated with hydrogen and Pd/C in a manner similar to Example 1, Step 7 to provide tert-butyl (1R,3 S,5r,7r)-2-(1-(4-(4-((3-aminopropyl)(tert-butoxycarbonyl)amino)piperidin-1-yl)-2-isopropylphenyl)-5-(2,6-dimethoxyphenyl)-1H-pyrazole-3-carboxamido)adamantane-2-carboxylate (20 mg, 23 μmol, 93%) as a white solid.

tert-Butyl (1R,3S,5r,7r)-2-(1-(4-(4-((3-aminopropyl)(tert-butoxycarbonyl)amino)piperidin-1-yl)-2-isopropylphenyl)-5-(2,6-dimethoxyphenyl)-1H-pyrazole-3-carboxamido)adamantane-2-carboxylate was treated with (((9H-fluoren-9-yl)methoxy)carbonyl)(sulfo)-D-alanine in a manner similar to Example 101, Step 2, then treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″(10-(2-(((S)-1-((3-((1-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-4-yl)amino)propyl)amino)-1-oxopropan-2-yl)amino)-2-oxoethyl)-1,4,7,10-tetraazacyclododecane-1,4,7-triyl)triacetic acid (1.4 mg, 1.0 μmol, 10%) as a white solid. MS: Calc'd for C₅₉H₈₅N₁₀O₁₆S: 1235.59, found [M+H]⁺: 1236.6

### Example 139: Compound 139

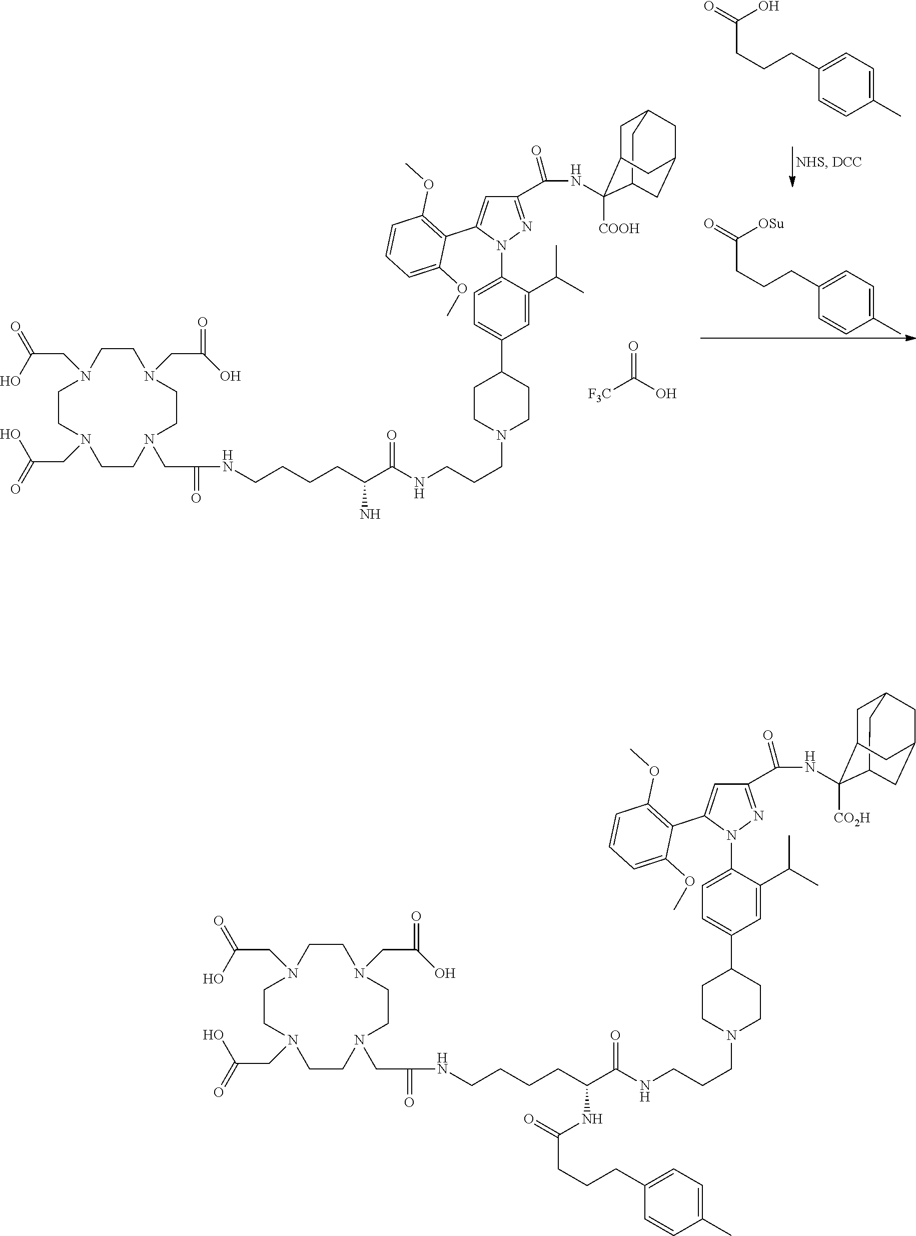

Step 1: To a mixture of 4-(p-tolyl)butanoic acid (1.0 g, 1 Eq, 5.6 mmol), N-hydroxysuccinimide (0.97 g, 1.5 Eq, 8.4 mmol) and DCC (1.7 g, 1.5 Eq, 8.2 mmol), to which was added THF (10 mL). The reaction mixture was stirred at 25° C. for 1 hour. The mixture was concentrated under reduced pressure then the crude product was purified by Prep-HPLC to afford 2,5-dioxopyrrolidin-1-yl 4-(p-tolyl)butanoate (300 mg, 1.09 mmol, 19%) as a white solid.

Step 2: To a mixture of 2,5-dioxopyrrolidin-1-yl 4-(p-tolyl)butanoate (12 mg, 3.1 Eq, 44 μmol), DIEA (5.5 mg, 7.4 μL, 3.0 Eq, 43 μmol) and DMF (2 mL) was added 2,2′,2″-(10-(2-(((R)-5-amino-6-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-6-oxohexyl)amino)-2-oxoethyl)-1,4,7,10-tetraazacyclododecane-1,4,7-triyl)triacetic acid (Compound 25) (17 mg, 1 Eq, 14 μmol) and the reaction mixture was stirred at 25° C. for 2 hours. The crude mixture was directly purified by Prep-HPLC to afford the 2,2,2-trifluoroacetic acid salt of 2,2′,2″-(10-(2-(((R)-6-((3-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-6-oxo-5-(4-(p-tolyl)butanamido)hexyl)amino)-2-oxoethyl)-1,4,7,10-tetraazacyclododecane-1,4,7-triyl)triacetic acid (7.8 mg, 5.3 μmol, 37%) as a white solid. MS: Calc'd for C₇₃H₁₀₃N₁₀O₁₄: 1357.77, found [M+H]⁺: 1358.7.

### Example 140: Compound 140

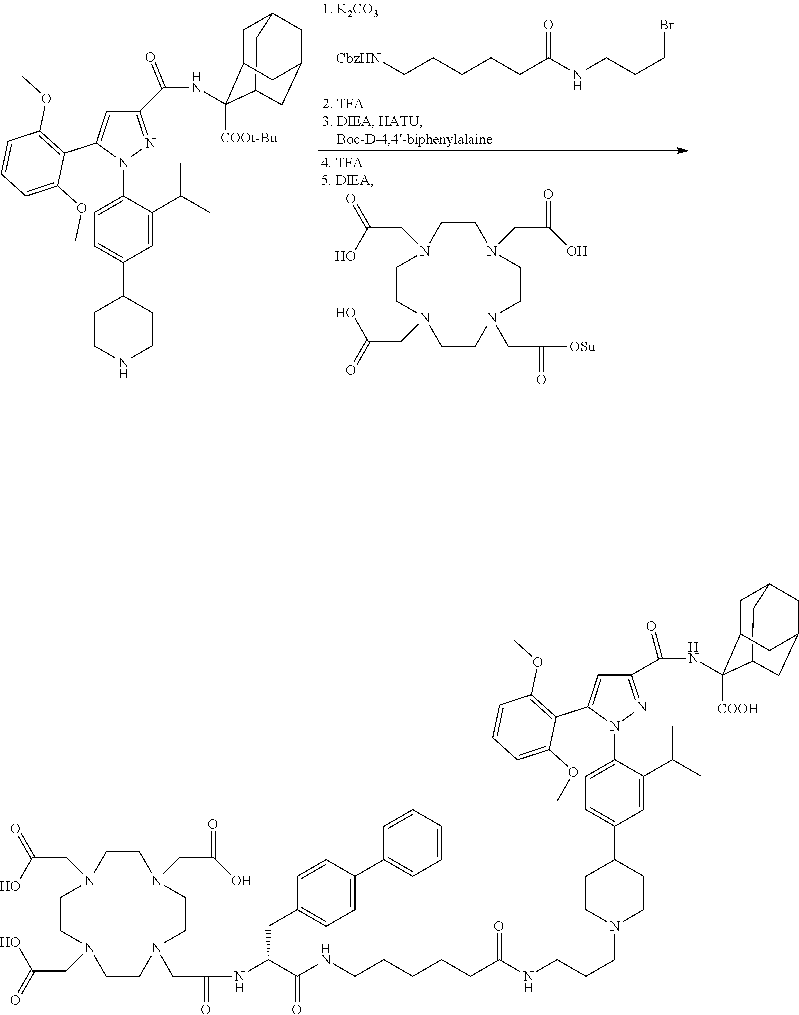

Step 1: To a mixture of tert-butyl (1R,3S,5r,7r)-2-(5-(2,6-dimethoxyphenyl)-1-(2-isopropyl-4-(piperidin-4-yl)phenyl)-1H-pyrazole-3-carboxamido)adamantane-2-carboxylate (110 mug, 1 Eq, 161 kmol), K₂CO₃ (44.5 mg, 2.00 Eq, 322 kmol) and DMF (2 mL), was added benzyl (6-((3-bromopropyl)amino)-6-oxohexyl)carbamate (186 mg, 3.00 Eq, 483 μmol). The reaction mixture was stirred at 80° C. for 2 h then the crude product was directly purified by Prep-HPLC to afford tert-butyl (1R,3S,5r,7r)-2-(1-(4-(1-(3-(6-(((benzyloxy)carbonyl)amino)hexanamido)propyl)piperidin-4-yl)-2-isopropylphenyl)-5-(2,6-dimethoxyphenyl)-1H-pyrazole-3-carboxamido)adamantane-2-carboxylate (100 mg, 101 μmol, 62.9%) as a white solid. [M+Na]⁺=1009.8.

Step 2: tert-butyl (1R,3S,5r,7r)-2-(1-(4-(1-(3-(6-(((benzyloxy)carbonyl)amino)hexanamido)propyl)piperidin-4-yl)-2-isopropylphenyl)-5-(2,6-dimethoxyphenyl)-1H-pyrazole-3-carboxamido)adamantane-2-carboxylate was treated with TFA in a manner similar to Example 1, Step 8 to provide (1R,3S,5r,7r)-2-(1-(4-(1-(3-(6-aminohexanamido)propyl)piperidin-4-yl)-2-isopropylphenyl)-5-(2,6-dimethoxyphenyl)-1H-pyrazole-3-carboxamido)adamantane-2-carboxylic acid (62 mg, 78 μmol, 77%) as a white crude solid, which was used directly in the next step without any purification.

Steps 3-5: (1R,3S,5r,7r)-2-(1-(4-(1-(3-(6-Aminohexanamido)propyl)piperidin-4-yl)-2-isopropylphenyl)-5-(2,6-dimethoxyphenyl)-1H-pyrazole-3-carboxamido)adamantane-2-carboxylic acid was treated with (R)-3-([1,1′-biphenyl]-4-yl)-2-((tert-butoxycarbonyl)amino)propanoic acid, HATU and DIEA in a manner similar to Example 103, Step 5, then treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2′-(10-(2-(((2R)-3-([1,1′-biphenyl]-4-yl)-1-((6-((3-(4-(4-(3-(((1R,2r,3S,5r)-2-carboxyadamantan-2-yl)carbamoyl)-5-(2,6-dimethoxyphenyl)-1H-pyrazol-1-yl)-3-isopropylphenyl)piperidin-1-yl)propyl)amino)-6-oxohexyl)amino)-1-oxopropan-2-yl)amino)-2-oxoethyl)-1,4,7,10-tetraazacyclododecane-1,4,7-triyl)triacetic acid (48.2 mg, 31.7 μmol, 60%) as a white solid. MS: Calc'd for C₇₇H₁₀₃N₁₀O₁₄: 1405.77, found [M+H]⁺: 1406.6

### Example 141: Compound 141

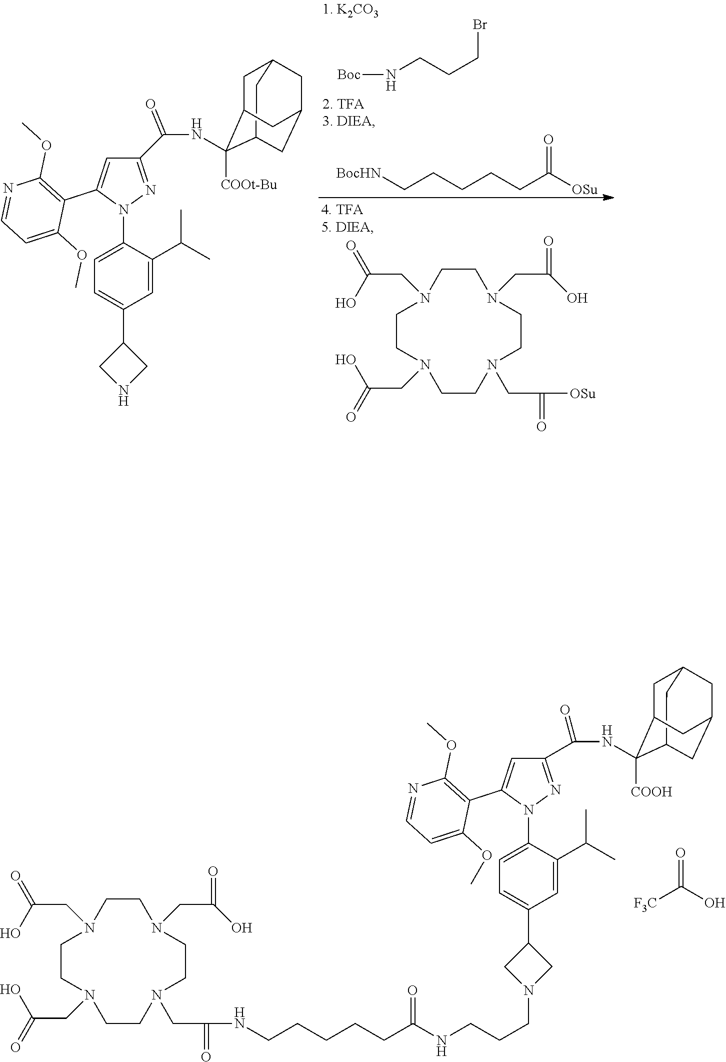

Steps 1-2: (1R,3S,5r,7r)-2-(1-(4-(1-(3-aminopropyl)azetidin-3-yl)-2-isopropylphenyl)-5-(2,4-dimethoxypyridin-3-yl)-1H-pyrazole-3-carboxamido)adamantane-2-carboxylic acid was synthesized from tert-butyl (1R,3S,5r,7r)-2-(1-(4-(azetidin-3-yl)-2-isopropylphenyl)-5-(2,4-dimethoxypyridin-3-yl)-1H-pyrazole-3-carboxamido)adamantane-2-carboxylate (Example 113, Step 2) and tert-butyl (3-bromopropyl)carbamate in manner similar to Example 113, Steps 3-4 to provide the product (40 mg, 61 μmol, 83%) as a white solid. [M+H]⁺=657.4.

Step 3: A mixture of 2,5-dioxopyrrolidin-1-yl 6-((tert-butoxycarbonyl)amino)hexanoate (40 mg, 2.0 Eq, 0.12 mmol), DIEA (25 mg, 3.2 Eq, 0.19 mmol), (1R,3S,5r,7r)-2-(1-(4-(1-(3-aminopropyl)azetidin-3-yl)-2-isopropylphenyl)-5-(2,4-dimethoxypyridin-3-yl)-1H-pyrazole-3-carboxamido)adamantane-2-carboxylic acid (40 mg, 1 Eq, 61 mmol) and DMF (4 mL) was stirred at 25° C. for 1 hour. The mixture was directly purified by MPLC to afford (1R,3S,5r,7r)-2-(1-(4-(1-(3-aminopropyl)azetidin-3-yl)-2-isopropylphenyl)-5-(2,4-dimethoxypyridin-3-yl)-1H-pyrazole-3-carboxamido)adamantane-2-carboxylic acid (40 mg, 61 μmol, 83%) as a white solid.

Steps 4-5: (1R,3S,5r,7r)-2-(1-(4-(1-(3-Aminopropyl)azetidin-3-yl)-2-isopropylphenyl)-5-(2,4-dimethoxypyridin-3-yl)-1H-pyrazole-3-carboxamido)adamantane-2-carboxylic acid was treated with TFA, then 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Steps 4 and 5 to provide the 2,2,2-trifluoroacetic acid salt of 2,2′,2″-(10-(2-((6-((3-(3-(4-(3-(((1R,3 S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)propyl)amino)-6-oxohexyl)amino)-2-oxoethyl)-1,4,7,10-tetraazacyclododecane-1,4,7-triyl)triacetic acid (27.4 mg, 21.6 nmol, 53%) as a white solid. MS: Calc'd for C₅₉H₈₅N₁₁O₁₃: 1155.63, found [M+H]⁺: 1156.6

### Example 142: Compound 142

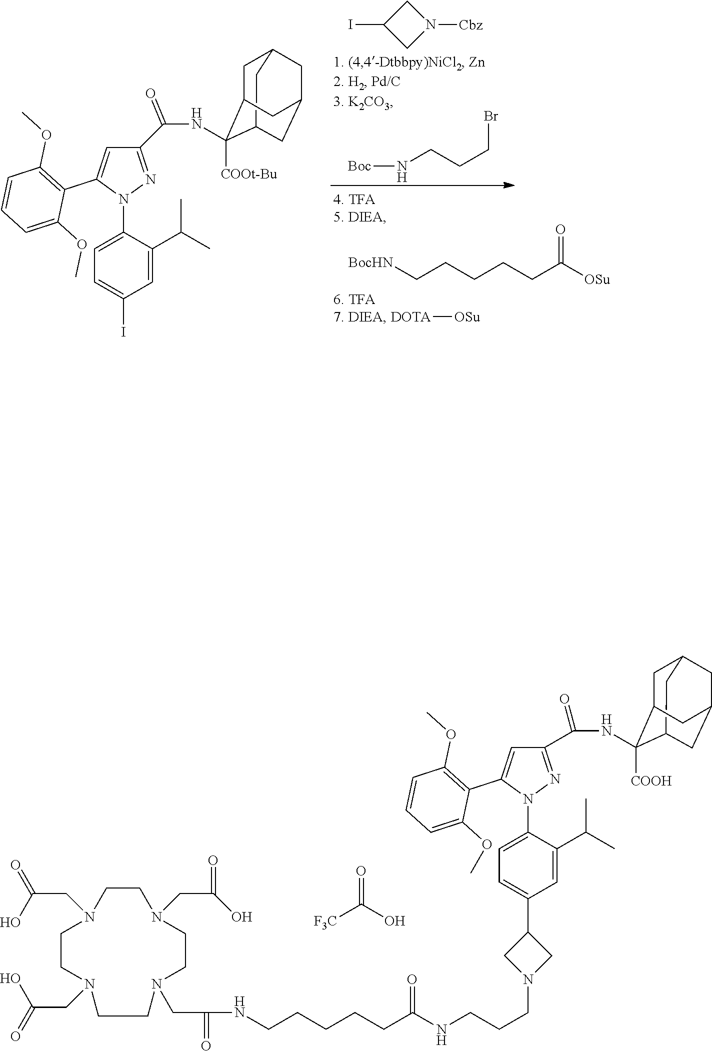

Step 1: A mixture of tert-butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (300 mg, 1 Eq, 413 μmol), benzyl 3-iodoazetidine-1-carboxylate (260 mg, 1.98 Eq, 820 μmol), [4,4′-Bis(1,1-dimethylethyl)-2,2′-bipyridine]nickel (II) dichloride (35 mg, 0.21 Eq, 88 μmol) and DME (3 mL) was stirred at 60° C. for 3 hours, then Zn (80 mg, 3.0 Eq, 1.2 mmol) was added and the reaction mixture was stirred at 60° C. for an additional 16 hours. The mixture was directly purified by MPLC to afford benzyl 3-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)azetidine-1-carboxylate (260 mg, 0.26 mmol, 64%) as a yellow solid.

Step 2: To a mixture of benzyl 3-(4-(3-(((1R,3S,5r,7r)-2-(tert-butoxycarbonyl)adamantan-2-yl)carbamoyl)-5-(2,6-dimethoxyphenyl)-1H-pyrazol-1-yl)-3-isopropylphenyl)azetidine-1-carboxylate (260 mg, 1 Eq, 330 μmol) and MeOH (3 mL) under N₂, was carefully added Pd—C (120 mg, 10% Wt, 0.342 Eq, 113 μmol). The flask was evacuated and flushed with hydrogen three times then the mixture was stirred for 1 hour at 25° C. under H₂. The reaction mixture was filtered through a pad of celite, the filtrate was concentrated and purified by MPLC to afford tert-butyl (1R,3S,5r,7r)-2-(1-(4-(azetidin-3-yl)-2-isopropylphenyl)-5-(2,6-dimethoxyphenyl)-1H-pyrazole-3-carboxamido)adamantane-2-carboxylate (220 mg, 0.27 mmol, 82%) as a white solid.

Steps 3-7: 2,2′,2″-(10-(2-((6-((3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)azetidin-1-yl)propyl)amino)-6-oxohexyl)amino)-2-oxoethyl)-1,4,7,10-tetraazacyclododecane-1,4,7-triyl)triacetic acid was synthesized from tert-butyl (1R,3S,5r,7r)-2-(1-(4-(azetidin-3-yl)-2-isopropylphenyl)-5-(2,6-dimethoxyphenyl)-1H-pyrazole-3-carboxamido)adamantane-2-carboxylate (90 mg, 1 Eq, 0.14 mmol) and tert-butyl (3-bromopropyl)carbamate (35 mg, 1.1 Eq, 0.15 mmol) in a manner similar to Example 141 to provide the product (31.5 mg, 25.1 μmol, 48%) as a white solid. MS: Calc'd for C₆₀H₈₆N₀O₁₃: 1154.64, found [M+H]⁺: 1155.7.

### Synthesis of Compound 142-Lu

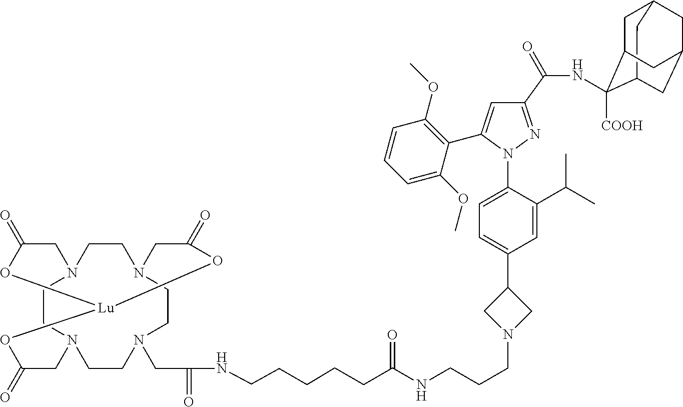

A mixture of 2,2′,2″-(10-(2-((6-((3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)azetidin-1-yl)propyl)amino)-6-oxohexyl)amino)-2-oxoethyl)-1,4,7,10-tetraazacyclododecane-1,4,7-triyl)triacetic acid-2,2,2-trifluoroacetic acid (1/1) (25 mg, 1 Eq, 20 μmol), lutetium(II) chloride (30 mg, 5.4 Eq, 0.11 mmol) and NaOAc/AcOH buffer (1 mL) was stirred at 80° C. for 30 min. The crude product was purified by Prep-HPLC to afford the title compound (21.7 mg, 15.1 μmol, 76%) as a white solid. MS: Calc'd for C₆₀H₈₃LuN₁₀O₁₃: 1326.55, found [M+H]⁺: 1327.9.

### Synthesis of Compound 142-Ga

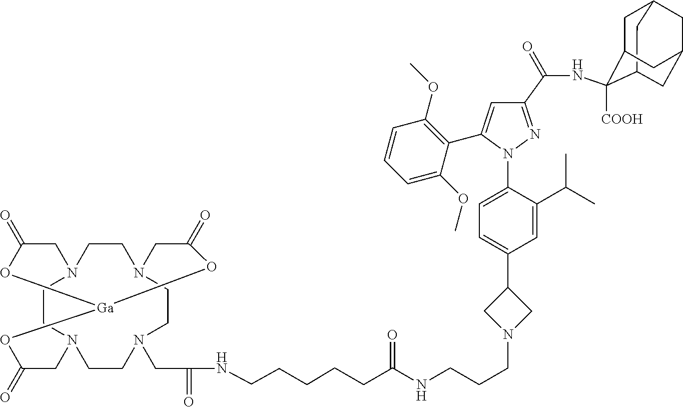

A mixture of 2,2′,2″-(10-(2-((6-((3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)azetidin-1-yl)propyl)amino)-6-oxohexyl)amino)-2-oxoethyl)-1,4,7,10-tetraazacyclododecane-1,4,7-triyl)triacetic acid-2,2,2-trifluoroacetic acid (1/1) (25 mg, 1 Eq, 20 μmol), gallium trichloride (20 mg, 5.8 Eq, 0.11 mmol) and NaOAc/AcOH buffer (2 mL) was stirred at 80° C. for 30 min. The crude product was purified by Prep-HPLC to afford the title compound (20.0 mg, 15.0 μmol, 76%) as a white solid. MS: Calc'd for C₆₀H₈₃GaN₁₀O₁₃: 1220.54, found [M+H]⁺: 1221.8.

### Example 143: Compound 143

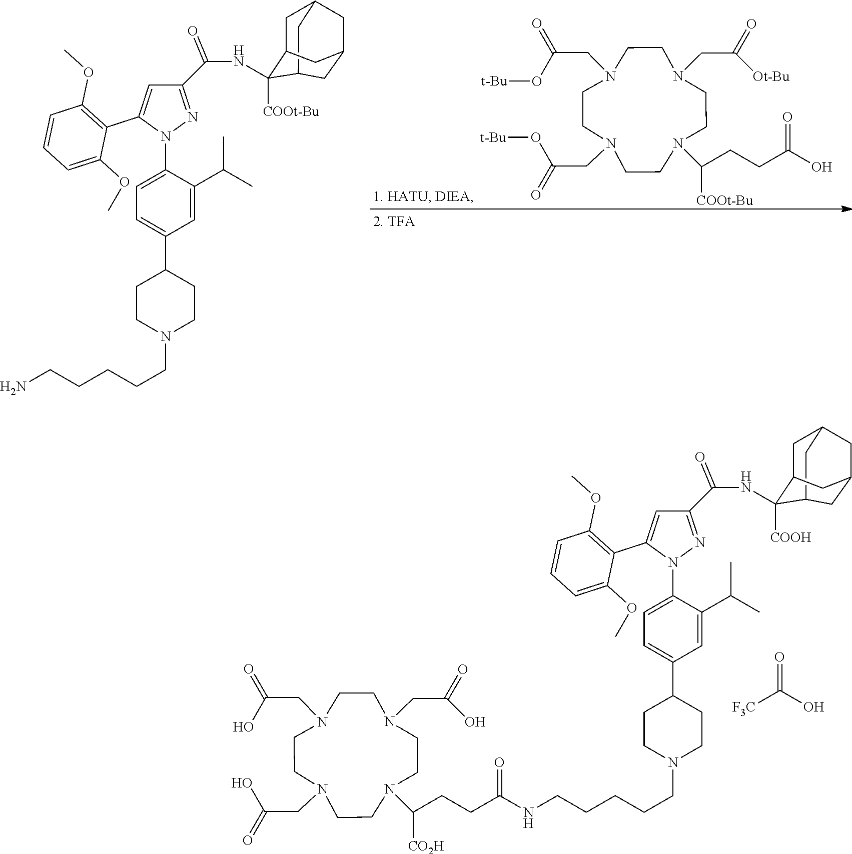

tert-Butyl(1R,3S,5r,7r)-2-(1-(4-(1-(5-aminopentyl)piperidin-4-yl)-2-isopropylphenyl)-5-(2,6-dimethoxyphenyl)-1H-pyrazole-3-carboxamido)adamantane-2-carboxylate (from Example 105, Step 2) was treated with 5-(tert-butoxy)-5-oxo-4-(4,7,10-tris(2-(tert-butoxy)-2-oxoethyl)-1,4,7,10-tetraazacyclododecan-1-yl)pentanoic acid then TFA in a manner similar to Example 120 to provide the 2,2,2-trifluoroacetic acid salt of 2,2′,2″-(10-(1-carboxy-4-((5-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)pentyl)amino)-4-oxobutyl)-1,4,7,10-tetraazacyclododecane-1,4,7-triyl)triacetic acid (48.6 mg, 37.8 μmol, 78%) as a white solid. MS: Calc'd for C₆₁H₈₈₇N₉O₁₄: 1169.64, found [M+H]⁺: 1170.7

### Example 144: Compound 144

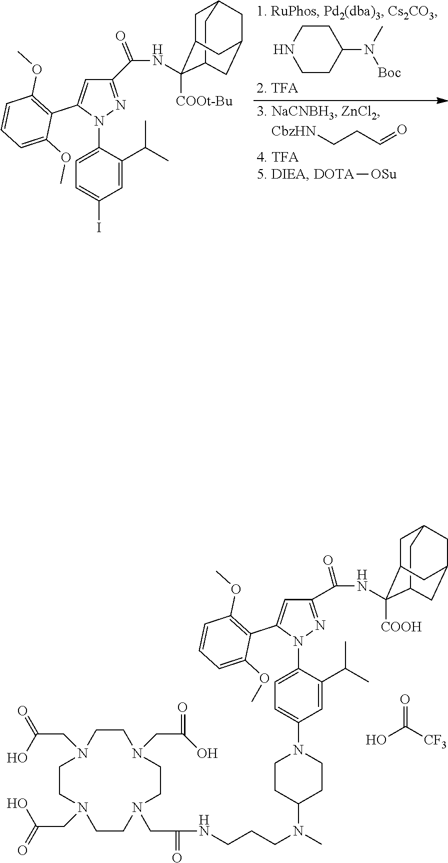

Step 1: tert-Butyl (1R,3S,5r,7r)-2-(5-(2,6-dimethoxyphenyl)-1-(4-iodo-2-isopropylphenyl)-1H-pyrazole-3-carboxamido)adamantane-2-carboxylate (100 mg, 1 Eq, 138 μmol) was treated with Pd₂(dba)₃ (13 mg, 0.10 Eq, 14 μmol), RuPhos (6.5 μg, 0.10 Eq, 14 μmol), Cs₂CO₃ (140 mg, 3.12 Eq, 430 μmol), and tert-butyl methyl(piperidin-4-yl)carbamate (40 mg, 1.4 Eq, 0.19 mmol) in a manner similar to Example 87, Step 1 to provide tert-butyl (1R,3S,5r,7r)-2-(1-(4-(4-((tert-butoxycarbonyl)(methyl)amino)piperidin-1-yl)-2-isopropylphenyl)-5-(2,6-dimethoxyphenyl)-1H-pyrazole-3-carboxamido)adamantane-2-carboxylate (80 mg, 99 μmol, 71%) as a white solid.

Steps 2-3: tert-Butyl (1R,3S,5r,7r)-2-(1-(4-(4-((tert-butoxycarbonyl)(methyl)amino)piperidin-1-yl)-2-isopropylphenyl)-5-(2,6-dimethoxyphenyl)-1H-pyrazole-3-carboxamido)adamantane-2-carboxylate was treated with TFA in a manner similar Example 1, Step 8, then treated with benzyl (3-oxopropyl)carbamate (60 mg, 3.5 Eq, 0.29 mmol) ZnCl₂ (20 mg, 1.7 Eq, 0.15 mmol) and sodium cyanoborohydride (20 mg, 19 μL, 3.8 Eq, 0.32 mmol) in a manner similar to Example 138, Step 1 to provide (1R,3 S,5r,7r)-2-(1-(4-(4-((3-(((benzyloxy)carbonyl)amino)propyl)(methyl)amino)piperidin-1-yl)-2-isopropylphenyl)-5-(2,6-dimethoxyphenyl)-1H-pyrazole-3-carboxamido)adamantane-2-carboxylic acid (35 mg, 41 μmol, 49%) as an off-white solid. [M+H]=847.4.

Steps 4-5: (1R,3S,5r,7r)-2-(1-(4-(4-((3-(((benzyloxy)carbonyl)amino)propyl)(methyl)amino)piperidin-1-yl)-2-isopropylphenyl)-5-(2,6-dimethoxyphenyl)-1H-pyrazole-3-carboxamido)adamantane-2-carboxylic acid was treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2-(10-(2-((3-((1-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-4-yl)(methyl)amino)propyl)amino)-2-oxoethyl)-1,4,7,10-tetraazacyclododecane-1,4,7-triyl)triacetic acid (29.9 mg, 24.6 μmol, 59%) as a white solid. MS: Calc'd for C₅₇H₈₂N₁₀O₁₂: 1098.61, found [M+H]⁺: 1099.5.

### Example 145: Compound 145

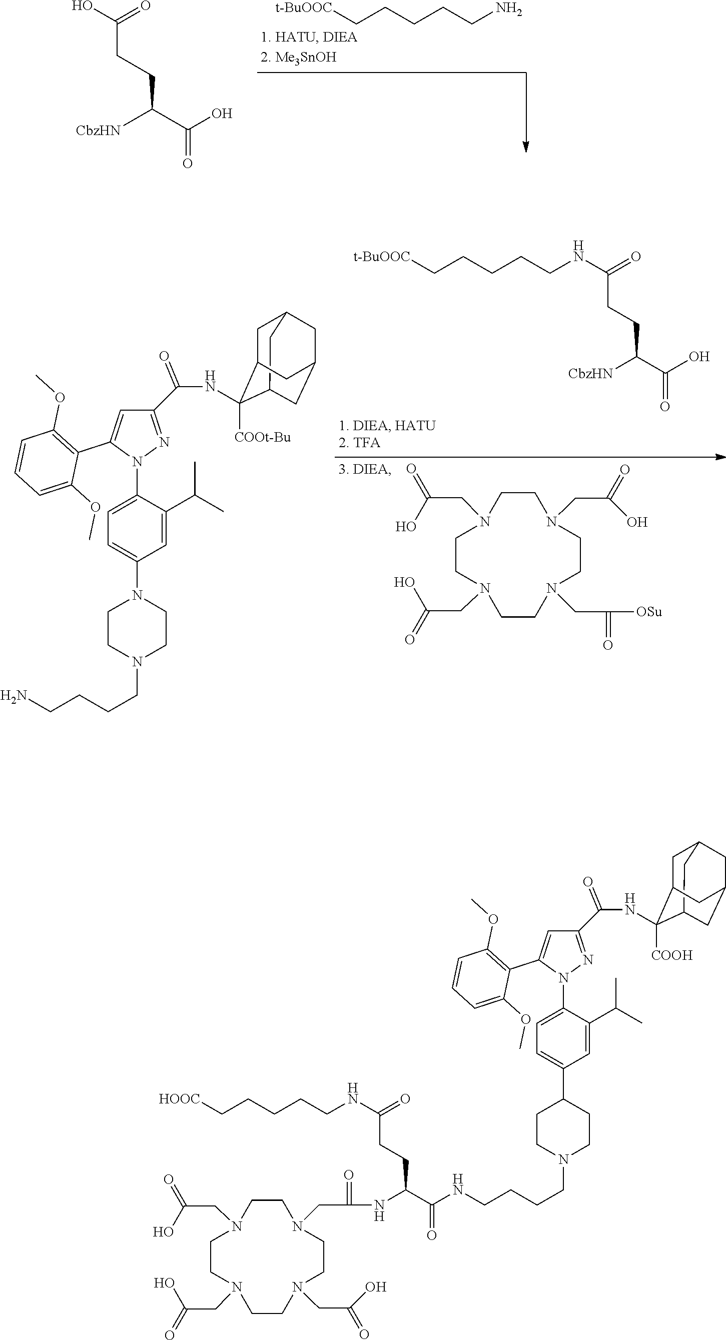

Step 1: A mixture of (S)-4-(((benzyloxy)carbonyl)amino)-5-methoxy-5-oxopentanoic acid (300 mg, 1 Eq, 1.02 mmol), HATU (425 mg, 1.10 Eq, 1.12 mmol), DIEA (394 mg, 531 μL, 3.00 Eq, 3.05 mmol) and DMF (20 mL) was stirred at 20° C. for 10 minutes, then tert-butyl 6-aminohexanoate (209 mg, 1.10 Eq, 1.12 mmol) was added and the reaction mixture was stirred at 25° C. for an additional 1 hour, then the mixture was quenched with water and purified directly by MPLC to afford tert-butyl (S)-6-(4-(((benzyloxy)carbonyl)amino)-5-methoxy-5-oxopentanamido)hexanoate (280 mg, 603 nmol, 59.3%) as an off-white oil. [M+H]⁺=465.2.

Step 2: A mixture of tert-butyl (S)-6-(4-(((benzyloxy)carbonyl)amino)-5-methoxy-5-oxopentanamido)hexanoate (300 mg, 1 Eq, 646 μmol), trimethyltinhydroxide (584 mg, 5.00 Eq, 3.23 mmol) and DCE (10 mL) was stirred at 90° C. for 16 hours. The mixture was directly purified by MPLC to afford N2-((benzyloxy)carbonyl)-N5-(6-(tert-butoxy)-6-oxohexyl)-L-glutamine (180 mg, 0.32 mmol, 49%) as a brown solid. [M+H]⁺=451.2.

Steps 3-5: tert-Butyl (1R,3S,5r,7r)-2-(1-(4-(1-(4-aminobutyl)piperidin-4-yl)-2-isopropylphenyl)-5-(2,6-dimethoxyphenyl)-1H-pyrazole-3-carboxamido)adamantane-2-carboxylate was treated with N2-((benzyloxy)carbonyl)-N5-(6-(tert-butoxy)-6-oxohexyl)-L-glutamine, HATU and DIEA in a manner similar to Example 103, Step 5, then treated with TFA at 60° C. in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2-(10-(2-(((S)-1-((4-(4-(4-(3-(((1R,3 S,5r,7r)-2-carboxyadamantan-2-yl)carbamoyl)-5-(²,6-dimethoxyphenyl)-1H-pyrazol-1-yl)-3-isopropylphenyl)piperidin-1-yl)butyl)amino)-5-((5-carboxypentyl)amino)-1,5-dioxopentan-2-yl)amino)-2-oxoethyl)-1,4,7,10-tetraazacyclododecane-1,4,7-triyl)triacetic acid (23.2 mg, 16.1 μmol, 43%) as a white solid.

MS: Calc'd for C₆₈H₉₉N₁₁O₁₆: 1325.73, found [M+H]⁺: 1326.7

### Example 146: Compound 146

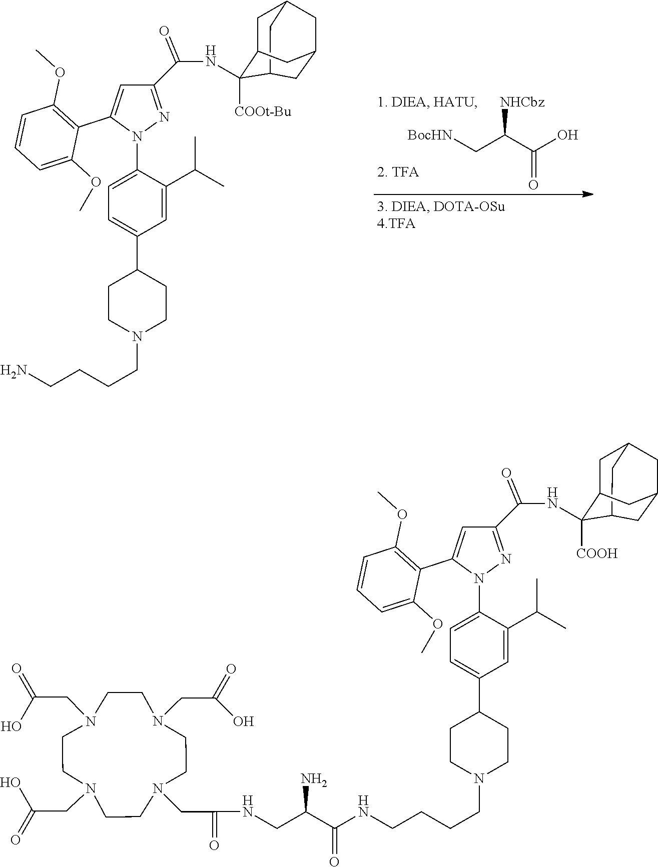

Steps 1-3: tert-butyl (1R,3S,5r,7r)-2-(1-(4-(1-(4-aminobutyl)piperidin-4-yl)-2-isopropylphenyl)-5-(2,6-dimethoxyphenyl)-1H-pyrazole-3-carboxamido)adamantane-2-carboxylate (55 mg, 1 Eq, 73 μmol) was treated with (R)-2-(((benzyloxy)carbonyl)amino)-3-((tert-butoxycarbonyl)amino)propanoic acid (30 mg, 1.2 Eq, 89 μmol), HATU and DIEA in a manner similar to Example 103, Step 5, then treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford 2,2′,2″-(10-(2-(((R)-2-(((benzyloxy)carbonyl)amino)-3-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)butyl)amino)-3-oxopropyl)amino)-2-oxoethyl)-1,4,7,10-tetraazacyclododecane-1,4,7-triyl)triacetic acid (40 mg, 31 μmol, 63%) as white solid.

Step 4: A mixture of 2,2′,2″-(10-(2-(((R)-2-(((benzyloxy)carbonyl)amino)-3-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)butyl)amino)-3-oxopropyl)amino)-2-oxoethyl)-1,4,7,10-tetraazacyclododecane-1,4,7-triyl)triacetic acid (40 mg, 1 Eq, 31 mol) and TFA (1 mL) was stirred at 60° C. for 1 hour. The mixture was concentrated under reduced pressure and the crude product was purified by Prep-HPLC to afford the 2,2,2-trifluoroacetic acid salt of 2,2′,2″-(10-(2-(((R)-2-amino-3-((4-(4-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)piperidin-1-yl)butyl)amino)-3-oxopropyl)amino)-2-oxoethyl)-1,4,7,10-tetraazacyclododecane-1,4,7-triyl)triacetic acid (29.7 mg, 23.1 μmol, 75%) as a white solid. MS: Calc'd for C₆₀H₈₇N₁₁O₁₃: 1169.65, found [M+H]⁺: 1170.6.

### Example 147: Compound 147

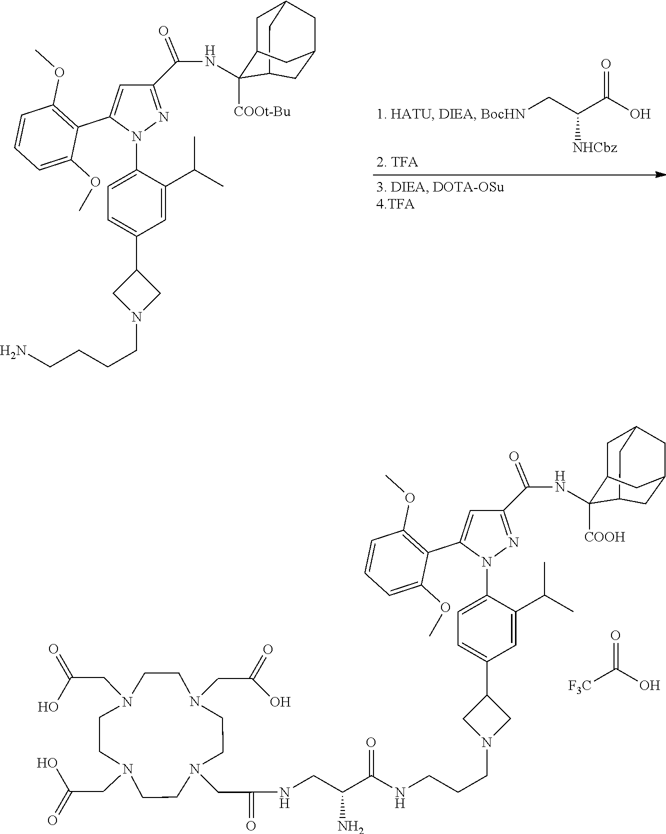

Steps 1-3: (1R,3S,5r,7r)-2-(1-(4-(1-(3-aminopropyl)azetidin-3-yl)-2-isopropylphenyl)-5-(2,6-dimethoxyphenyl)-1H-pyrazole-3-carboxamido)adamantane-2-carboxylic acid (75 mg, 80% Wt, 1 Eq, 91 μmol) was treated with (R)-2-(((benzyloxy)carbonyl)amino)-3-((tert-butoxycarbonyl)amino)propanoic acid (37 mg, 1.2 Eq, 0.11 mmol), HATU and DIEA in a manner similar to Example 103, Step 5, then treated with TFA in a manner similar Example 1, Step 8, then treated with DIEA and 2,2′,2″(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford 2,2′,2″-(10-(2-(((R)-2-(((benzyloxy)carbonyl)amino)-3-((3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)azetidin-1-yl)propyl)amino)-3-oxopropyl)amino)-2-oxoethyl)-1,4,7,10-tetraazacyclododecane-1,4,7-triyl)triacetic acid (25 mg, 20 μmol, 39%) as a white solid. [M+H]⁺=1262.5.

Step 4: A mixture of 2,2′,2″-(10-(2-(((R)-2-(((benzyloxy)carbonyl)amino)-3-((3-(3-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)azetidin-1-yl)propyl)amino)-3-oxopropyl)amino)-2-oxoethyl)-1,4,7,10-tetraazacyclododecane-1,4,7-triyl)triacetic acid (25 mg, 1 Eq, 20 μmol) and TFA (1.5 mL) was stirred at 60° C. for 1 hour. The mixture was concentrated tinder reduced pressure and the crude product was purified by Prep-HPLC to afford the 2,2,2-trifluoroacetic acid salt of 2′,2″-(10-(2—(((R)-2-amino-3-((3-(3-(4-(3-(((1R,3 S,5r,7r)-2-carboxyadamantan-2-yl)carbamoyl)-5-(2,6-dimethoxyphenyl)-1H-pyrazol-1-yl)-3-isopropylphenyl)azetidin-1-yl)propyl)amino)-3-oxopropyl)amino)-2-oxoethyl)-1,4,7,10-tetraazacyclododecane-1,4,7-triyl)triacetic acid (15.0 mg, 12.1 μmol, 61%) as a white solid. MS: Calc'd for C₅₇H₈₁N₁₁O₁₃: 1127.60, found [M+H]⁺: 1128.5.

### Example 148: Compound 148

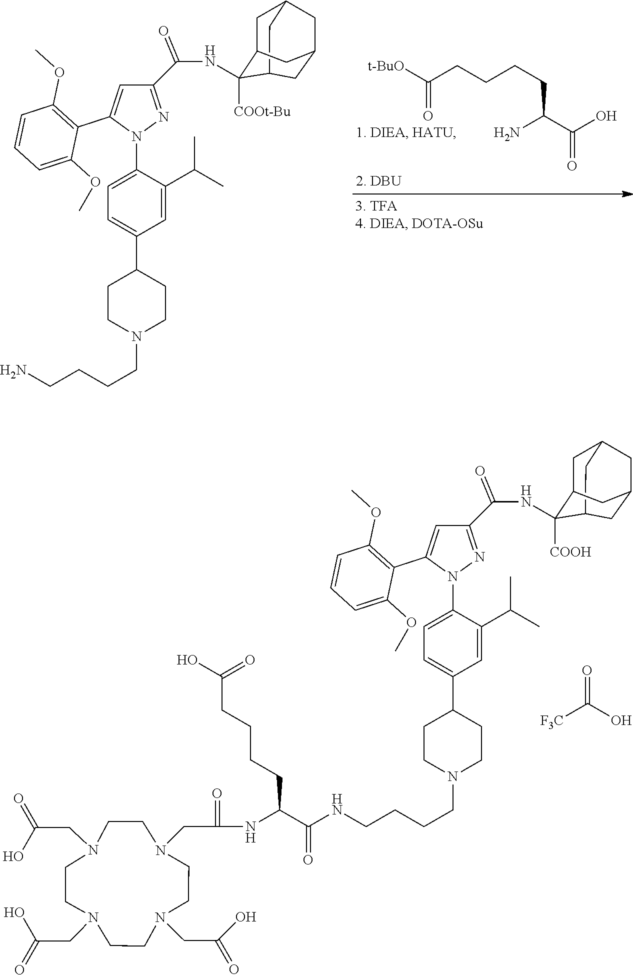

tert-Butyl (1R,3S,5r,7r)-2-(1-(4-(1-(4-aminobutyl)piperidin-4-yl)-2-isopropylphenyl)-5-(2,6-dimethoxyphenyl)-1H-pyrazole-3-carboxamido)adamantane-2-carboxylate (95 mg, 80% Wt, 1 Eq, 0.10 mmol) was treated with (S)-2-((((9H-fluoren-9-yl)methoxy)carbonyl)amino)-7-(tert-butoxy)-7-oxoheptanoic acid (50 mg, 1.1 Eq, 0.11 mmol), HATU and DIEA in a manner similar to Example 103, Step 5, then treated with DBU in a manner similar to Example 68, Step 5, then treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-(((S)-6-carboxy-1-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)butyl)amino)-1-oxohexan-2-yl)amino)-2-oxoethyl)-1,4,7,10-tetraazacyclododecane-1,4,7-triyl)triacetic acid (17.2 mg, 12.7 μmol, 54%) as a white solid. MS: Calc'd for C₆₄H₉₂N₁₀O₁₅: 1240.67, found [M+H]⁺: 1241.6.

### Example 149: Compound 149

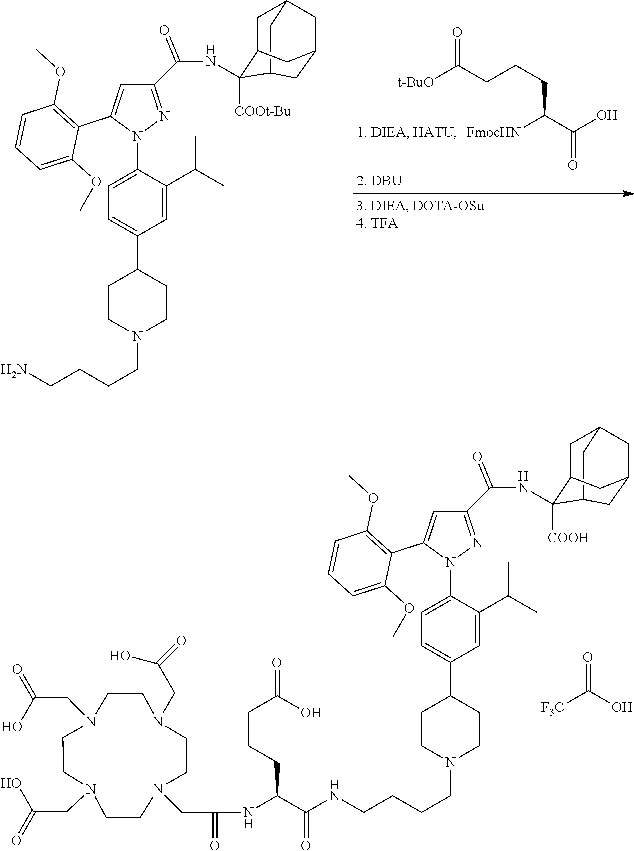

tert-Butyl (1R,3S,5r,7r)-2-(1-(4-(1-(4-aminobutyl)piperidin-4-yl)-2-isopropylphenyl)-5-(2,6-dimethoxyphenyl)-1H-pyrazole-3-carboxamido)adamantane-2-carboxylate (95 mg, 80% Wt, 1 Eq, 0.10 mmol) was treated with (S)-2-((((9H-fluoren-9-yl)methoxy)carbonyl)amino)-6-(tert-butoxy)-6-oxohexanoic acid (117 mg, 2.01 Eq, 266 μmol), HATU and DIEA in a manner similar to Example 103, Step 5, then treated with DBU in a manner similar to Example 68, Step 5, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5, then treated with TFA in a manner similar Example 1, Step 8, to afford the 2,2,2-trifluoroacetic acid salt of 2,2′,2″-(10-(2-(((S)-5-carboxy-1-((4-(4-(4-(3-(((1R,3S,5r,7r)-2-carboxyadamantan-2-yl)carbamoyl)-5-(2,6-dimethoxyphenyl)-1H-pyrazol-1-yl)-3-isopropylphenyl)piperidin-1-yl)butyl)amino)-1-oxopentan-2-yl)amino)-2-oxoethyl)-1,4,7,10-tetraazacyclododecane-1,4,7-triyl)triacetic acid (22.5 mg, 16.8 μmol, 90%) as a white solid. MS: Calc'd for C₆₃H₉₀N₁₀O₁₅: 1226.66, found [M+H]⁺: 1227.8.

### Example 150: Compound 150

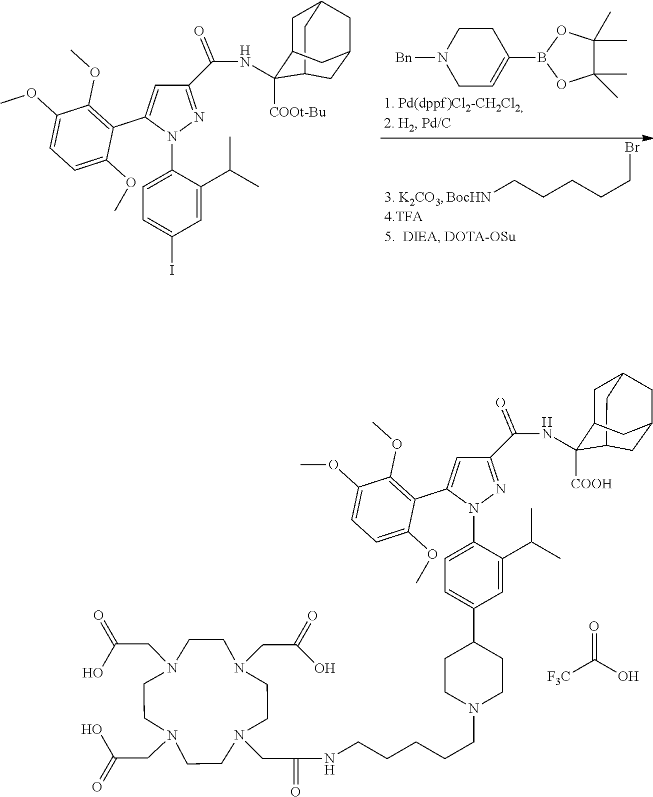

Step 1: A mixture of tert-butyl (1R,3 S,5r,7r)-2-(1-(4-iodo-2-isopropylphenyl)-5-(2,3,6-trimethoxyphenyl)-1H-pyrazole-3-carboxamido)adamantane-2-carboxylate (800 mg, 1 Eq, 1.06 mmol), 1-benzyl-4-(4,4,5,5-tetramethyl-1,3,2-dioxaborolan-2-yl)-1,2,3,6-tetrahydropyridine(400 mg, 1.26 Eq, 1.34 mmol), PdCl₂(dppf)-CH₂Cl₂ (50 mg, 0.058 Eq, 61 mol), K₂CO₃ (450 mg, 3.08 Eq, 3.26 mmol), 1,4-dioxane (10 mL) and H₂O (1 mL) under N₂ was stirred at 50° C. for 2 hours. The mixture was directly purified by MPLC to afford tert-butyl (1R,3S,5r,7r)-2-(1-(4-(1-benzyl-1,2,3,6-tetrahydropyridin-4-yl)-2-isopropylphenyl)-5-(2,3,6-trimethoxyphenyl)-1H-pyrazole-3-carboxamido)adamantane-2-carboxylate (680 mg, 849 μmol, 80.2%) as a yellow oil.

Steps 2-3: tert-Butyl (1R,3S,5r,7r)-2-(1-(4-(1-benzyl-1,2,3,6-tetrahydropyridin-4-yl)-2-isopropylphenyl)-5-(2,3,6-trimethoxyphenyl)-1H-pyrazole-3-carboxamido)adamantane-2-carboxylate (680 mg, 1 Eq, 849 mol) was treated with H₂ and Pd/C in a manner similar to Example 1, Step 7 then treated with K₂CO₃ and tert-butyl (5-bromopentyl)carbamate in a manner similar to Example 58, Step 2 to provide tert-butyl (1R,3S,5r,7r)-2-(1-(4-(1-(5-((tert-butoxycarbonyl)amino)pentyl)piperidin-4-yl)-2-isopropylphenyl)-5-(2,3,6-trimethoxyphenyl)-1H-pyrazole-3-carboxamido)adamantane-2-carboxylate (71 mg, 79 μmol, 70%) as a white solid.

Steps 4-5: tert-Butyl (1R,3S,5r,7r)-2-(1-(4-(1-(5-((tert-butoxycarbonyl)amino)pentyl)piperidin-4-yl)-2-isopropylphenyl)-5-(2,3,6-trimethoxyphenyl)-1H-pyrazole-3-carboxamido)adamantane-2-carboxylate was treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((5-(4-(4-(3-(((1R,3 S,5r,7r)-2-carboxyadamantan-2-yl)carbamoyl)-5-(2,3,6-trimethoxyphenyl)-1H-pyrazol-1-yl)-3-isopropylphenyl)piperidin-1-yl)pentyl)amino)-2-oxoethyl)-1,4,7,10-tetraazacyclododecane-1,4,7-triyl)triacetic acid (27.8 mg, 22.4 μmol, 23%) as a white solid. MS: Calc'd for C₅₉H₈₅N₉O₃: 1127.63, found [M+H]⁺: 1128.6.

### Example 151: Compound 151

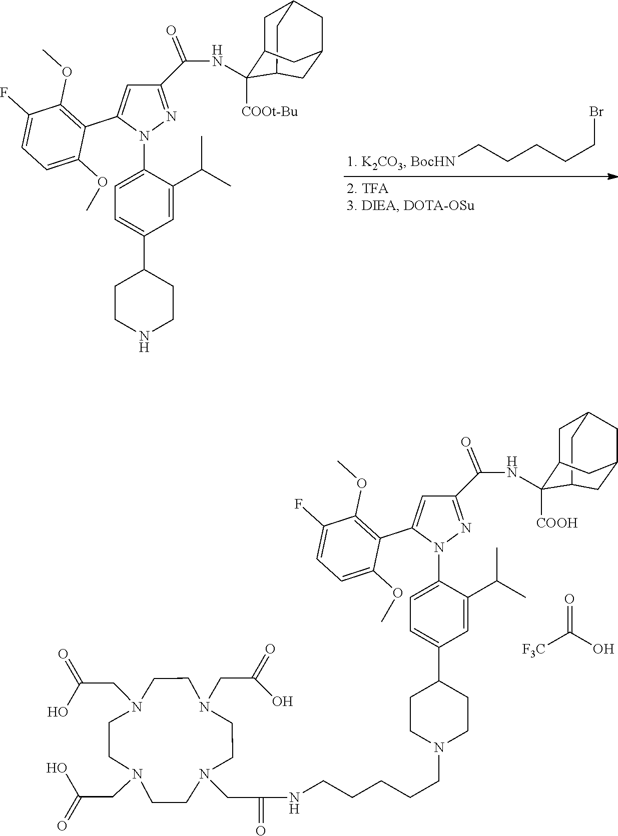

Steps 1-3: tert-Butyl (1R,3S,5r,7r)-2-(5-(3-fluoro-2,6-dimethoxyphenyl)-1-(2-isopropyl-4-(piperidin-4-yl)phenyl)-1H-pyrazole-3-carboxamido)adamantane-2-carboxylate (70 mg, 1 Eq, 0.10 mmol) was treated with K₂CO₃ and tert-butyl (5-bromopentyl)carbamate in a manner similar to Example 58, Step 2, treated with TFA in a manner similar Example 1, Step 8, then treated with DIEA and 2 2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((5-(4-(4-(3-(((1R,3S,5r,7r)-2-carboxyadamantan-2-yl)carbamoyl)-5-(3-fluoro-2,6-dimethoxyphenyl)-1H-pyrazol-1-yl)-3-isopropylphenyl)piperidin-1-yl)pentyl)amino)-2-oxoethyl)-1,4,7,10-tetraazacyclododecane-1,4,7-triyl)triacetic acid (74.0 mg, 60.1 μmol, 91%) as a white solid. MS: Calc'd for C₅₈H₈₂FN₉O₁₂: 1115.61, found [M+H]⁺: 1116.7.

### Example 152: Compound 152

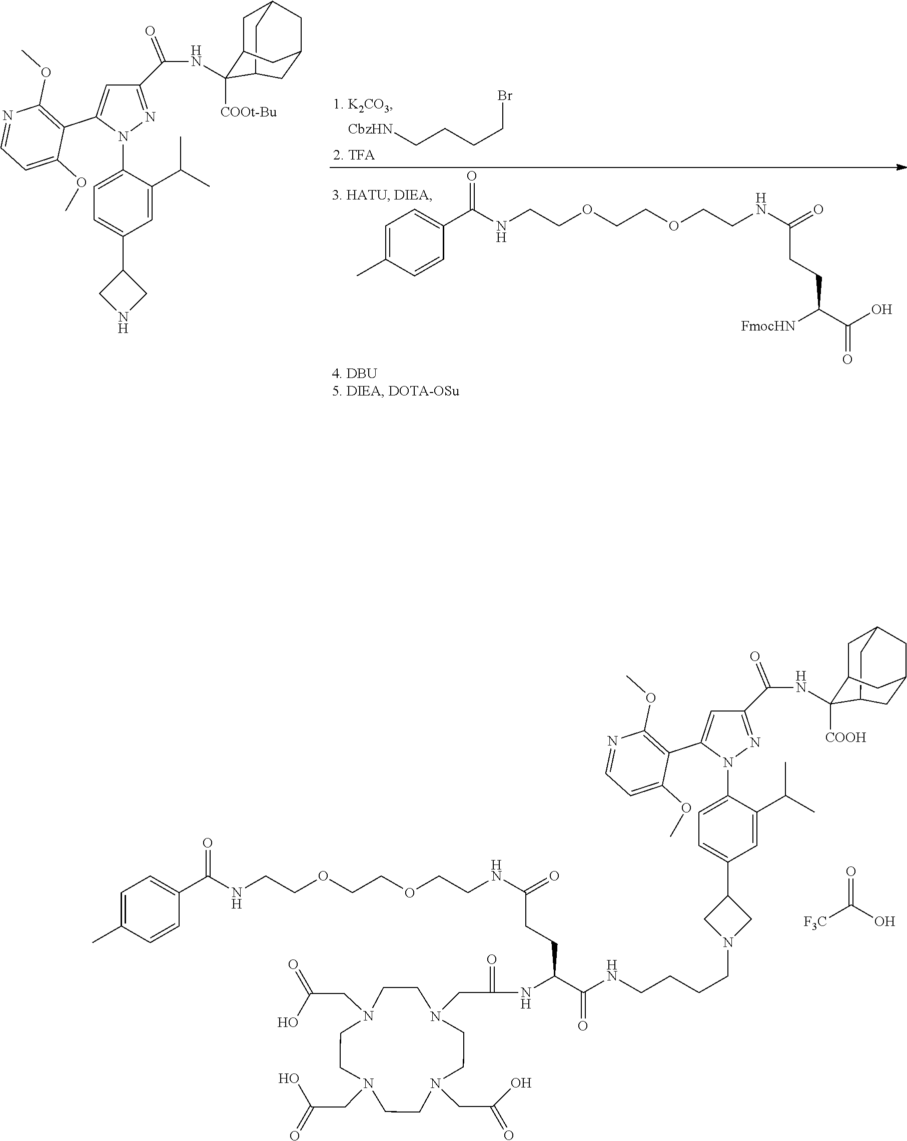

Steps 1-2: tert-Butyl (1R,3S,5r,7r)-2-(1-(4-(azetidin-3-yl)-2-isopropylphenyl)-5-(2,4-dimethoxypyridin-3-yl)-1H-pyrazole-3-carboxamido)adamantane-2-carboxylate (Example 113, Step 2) was treated with benzyl (4-bromobutyl)carbamate and K₂CO₃, in manner similar to Example 113, Step 3, then treated with TFA in a manner similar to Example 8, Step 6 to provide (1R,3 S,5r,7r)-2-(1-(4-(1-(4-aminobutyl)azetidin-3-yl)-2-isopropylphenyl)-5-(2,4-dimethoxypyridin-3-yl)-1H-pyrazole-3-carboxamido)adamantane-2-carboxylic acid (120 mg, 0.13 mmol, 83%) which was used directly in the next step without further purification. MS: Calc'd for C₃₈H₅₀N₆O₅: 670.38, found [M+H]⁺: 671.4.

Steps 3-5: (1R,3S,5r,7r)-2-(1-(4-(1-(4-aminobutyl)azetidin-3-yl)-2-isopropylphenyl)-5-(2,4-dimethoxypyridin-3-yl)-1H-pyrazole-3-carboxamido)adamantane-2-carboxylic acid (60 mg, 75% Wt, 1 Eq, 67 μmol) was treated with (S)-15-((((9H-fluoren-9-yl)methoxy)carbonyl)amino)-1,12-dioxo-1-(p-tolyl)-5,8-dioxa-2,11-diazahexadecan-16-oic acid (83 mg, 2.0 Eq, 0.13 mmol), HATU and DIEA in a manner similar to Example 103, Step 5, then treated with DBU in a manner similar to Example 68, Step 5, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-((15S)-15-((4-(3-(4-(3-(((1R,3S,5r,7r)-2-carboxyadamantan-2-yl)carbamoyl)-5-(2,4-dimethoxypyridin-3-yl)-1H-pyrazol-1-yl)-3-isopropylphenyl)azetidin-1-yl)butyl)carbamoyl)-1,12,17-trioxo-1-(p-tolyl)-5,8-dioxa-2,11,16-triazaoctadecan-18-yl)-1,4,7,10-tetraazacyclododecane-1,4,7-triyl)triacetic acid as a white solid. MS: Calc'd for C₇₃H₁₀₃N₁₃O₇: 1433.76, found [M+H]⁺: 1434.9.

### Example 153: Compound 153

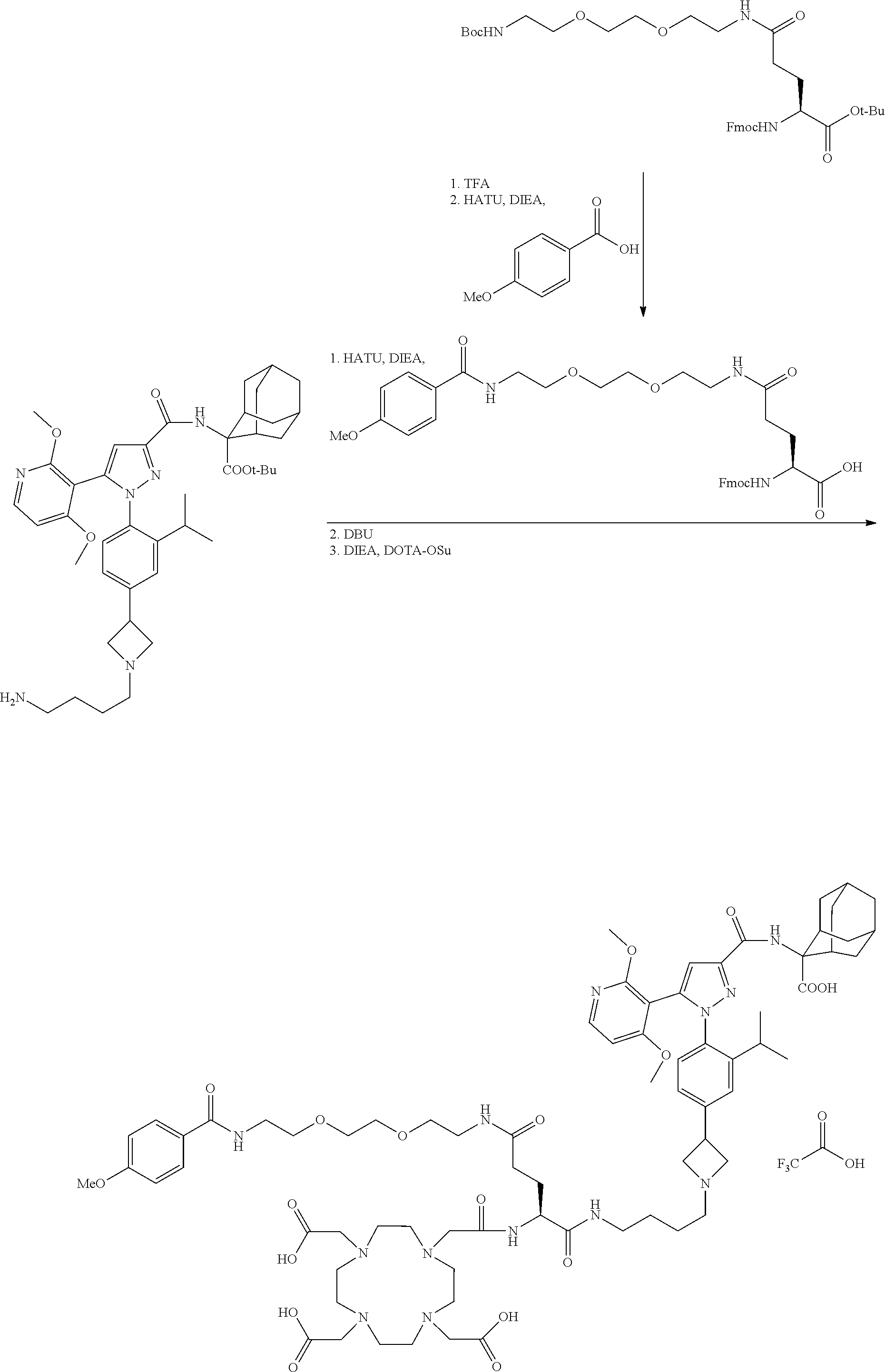

Step 1: To a mixture of tert-butyl (S)-18-((((9H-fluoren-9-yl)methoxy)carbonyl)amino)-2,2-dimethyl-4,15-dioxo-3,8,11-trioxa-5,14-diazanonadecan-19-oate (500 mg, 1 Eq, 762 μmol) and DCM (6 mL) was added TFA (2 mL). The reaction mixture was stirred at 25° C. for 1 hour. The mixture was concentrated under reduced pressure to afford N2-(((9H-fluoren-9-yl)methoxy)carbonyl)-N5-(2-(2-(2-aminoethoxy)ethoxy)ethyl)-L-glutamine (360 mg, 0.61 mmol, 80%) which was used directly in the next step without further purification. [M+H]⁺=500.1.

Step 2: A mixture of 4-methoxybenzoic acid (91 mg, 2.0 Eq, 0.60 mmol), HATU (171 mg, 1.50 Eq, 450 μmol), DIEA (116 mg, 156 μL, 2.99 Eq, 897 μmol) and DMF (3 mL) was stirred at 20° C. for 10 minutes, then N2-(((9H-fluoren-9-yl)methoxy)carbonyl)-N5-(2-(2-(2-aminoethoxy)ethoxy)ethyl)-L-glutamine(150 mg, 1 Eq, 300 μmol) was added and the reaction mixture was stirred at 25° C. for an additional 1 hour. The mixture was directly purified by MPLC to afford (S)-15-((((9H-fluoren-9-yl)methoxy)carbonyl)amino)-1-(4-methoxyphenyl)-1,12-dioxo-5,8-dioxa-2,11-diazahexadecan-16-oic acid as a yellow oil. [M+H]⁺=634.2.

Steps 3-5: (S)-15-((((9H-fluoren-9-yl)methoxy)carbonyl)amino)-1-(4-methoxyphenyl)-1,12-dioxo-5,8-dioxa-2,11-diazahexadecan-16-oic acid (60 mg, 75% Wt, 1 Eq, 67 μmol) was treated with (S)-15-((((9H-fluoren-9-yl)methoxy)carbonyl)amino)-1-(4-methoxyphenyl)-1,12-dioxo-5,8-dioxa-2,11-diazahexadecan-16-oic acid (83 mg, 2.0 Eq, 0.13 mmol), HATU and DIEA in a manner similar to Example 103, Step 5, then treated with DBU in a manner similar to Example 68, Step 5, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-((15S)-15-((4-(3-(4-(3-(((1R,3S,5r,7r)-2-carboxyadamantan-2-yl)carbamoyl)-5-(2,4-dimethoxypyridin-3-yl)-11H-pyrazol-1-yl)-3-isopropylphenyl)azetidin-1-yl)butyl)carbamoyl)-1-(4-methoxyphenyl)-1,12,17-trioxo-5,8-dioxa-2,11,16-triazaoctadecan-18-yl)-1,4,7,10-tetraazacyclododecane-1,4,7-triyl)triacetic acid as a white solid. MS: Calc'd for C₇₃H₁₀₃N₁₃O₁₈: 1449.75, found [M+H]⁺: 1450.6.

### Example 154: Compound 154

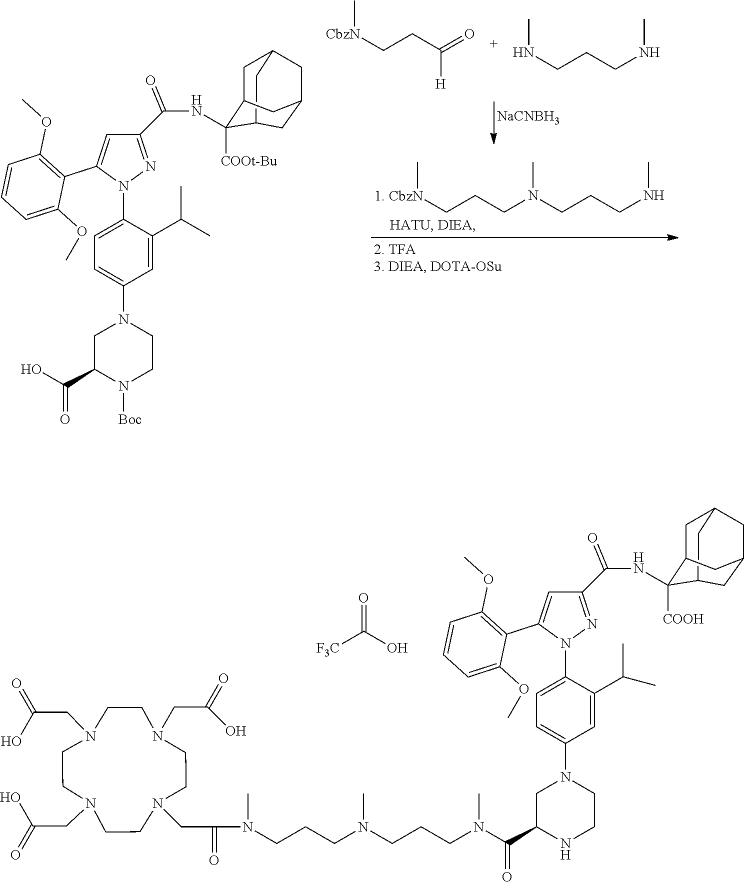

Step 1: A mixture of benzyl methyl(3-oxopropyl)carbamate (200 mg, 1 Eq, 904 μmol), N1,N3-dimethylpropane-1,3-diamine (93 mg, 1.0 Eq, 0.91 mmol) and MeOH (2 mL) was stirred at 20° C. for 10 minutes, then NaBH₃CN (114 mg, 2.01 Eq, 1.81 mmol) was added and the reaction mixture was stirred at 25° C. for an additional 1 hour. The mixture was directly purified by MPLC to afford benzyl methyl(3-(methyl(3-(methylamino)propyl)amino)propyl)carbamate (100 mg, 325 μmol, 36.0%) as a yellow oil.

A mixture of (R)-1-(tert-butoxycarbonyl)-4-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-2-carboxylic acid (70 mg, 1 Eq, 85 μmol), DIEA (37 mg, 50 μL, 3.4 Eq, 0.29 mmol), HATU (37 mg, 1.2 Eq, 97 μmol) and DMF (3 mL) was stirred at 20° C. for 10 minutes, then benzyl methyl(3-(methyl(3-(methylamino)propyl)amino)propyl)carbamate (30 mg 1.2 Eq, 98 μmol) was added and the reaction mixture was stirred at 25° C. for an additional 1 hour. The mixture was directly purified by MPLC to afford tert-butyl (R)-2-((3-((3-(((benzyloxy)carbonyl)(methyl)amino)propyl)(methyl)amino)propyl)(methyl)carbamoyl)-4-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-1-carboxylate (60 mg, 54 μmol, 64%) as a yellow oil. [M+H]⁺=1017.8.

tert-Butyl (R)-2-((3-((3-(((benzyloxy)carbonyl)(methyl)amino)propyl)(methyl)amino)propyl)(methyl)carbamoyl)-4-(4-(3-(((1R,3 S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-1-carboxylate was treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((3-((3-((R)-4-(4-(3-(((1R,3S,5R,7R)-2-carboxyadamantan-2-yl)carbamoyl)-5-(2,6-dimethoxyphenyl)-1H-pyrazol-1-yl)-3-isopropylphenyl)-N-methylpiperazine-2-carboxamido)propyl)(methyl)amino)propyl)(methyl)amino)-2-oxoethyl)-1,4,7,10-tetraazacyclododecane-1,4,7-triyl)triacetic acid as white solid. MS: Calc'd for C₆₂H₉₂N₁₂O₁₃: 1212.69, found [M+H]⁺: 1213.6.

### Example 155: Compound 155

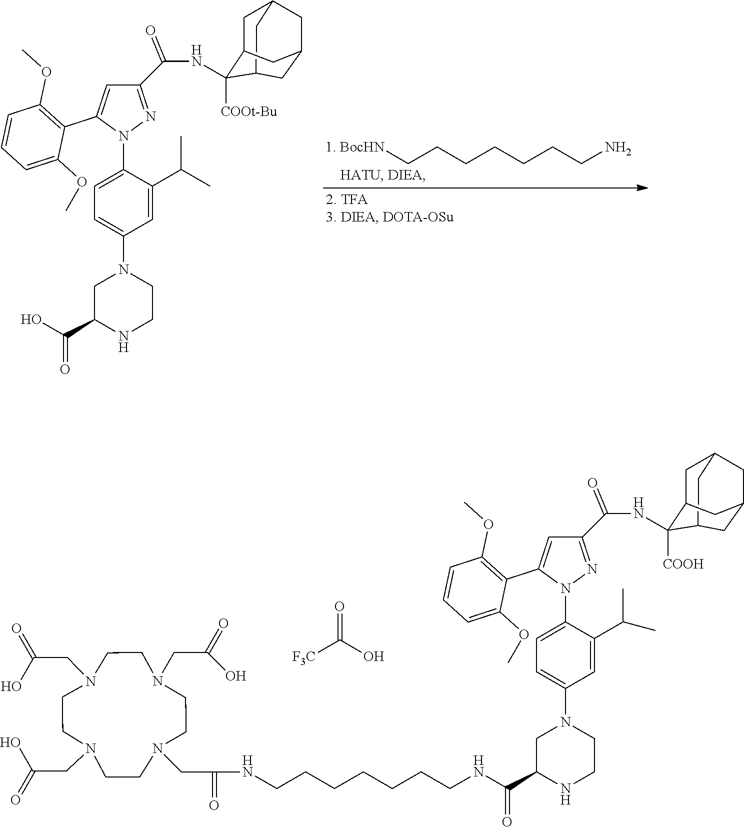

(R)-1-(tert-butoxycarbonyl)-4-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-2-carboxylic acid (70 mg, 1 Eq, 85 μmol) (from Example 48, Step 6) was treated with tert-butyl (7-aminoheptyl)carbamate (29 mg, 1.5 Eq, 0.13 mmol), HA TU and DIEA in a manner similar to Example 154, Step 2, treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(C-((7-((R)-4-(4-(3-(((1R,3 S,5R,7R)-2-carboxyadamantan-2-yl)carbamoyl)-5-(2,6-dimethoxyphenyl)-1H-pyrazol-1-yl)-3-isopropylphenyl)piperazine-2-carboxamido)heptyl)amino)-2-oxoethyl)-1,4,7,10-tetraazacyclododecane-1,4,7-triyl)triacetic acid (26.2 mg, 20.4 μmol, 53%) as a white solid. MS: Calc'd for C₆₀H₈₈₇N₁₁O₁₃: 1169.65, found [M+H]⁺: 1170.5.

### Example 156: Compound 156

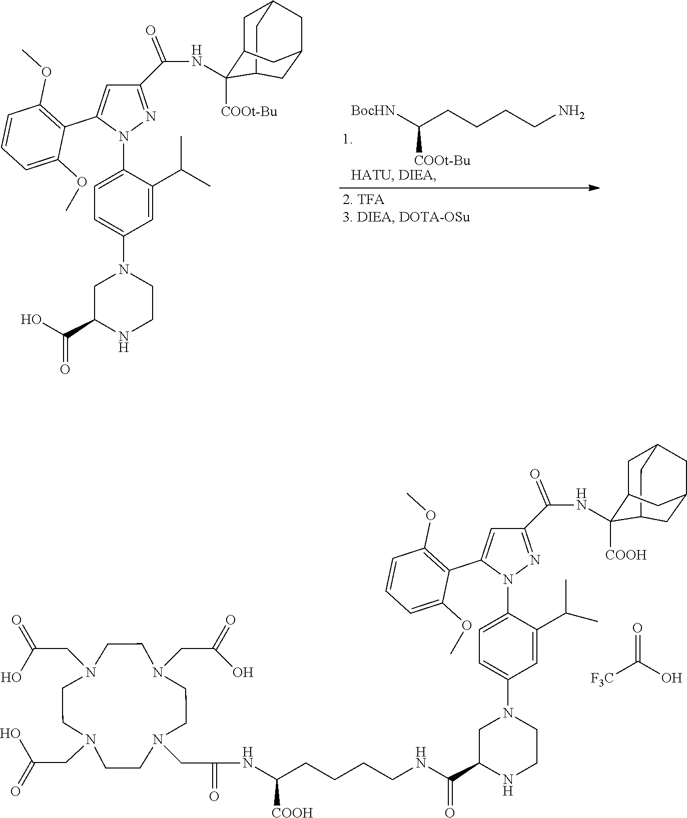

(R)-1-(tert-butoxycarbonyl)-4-(4-(3-(((1R,3S,5R,7R)-2-(tert-butoxycarbonyl)adamantan-2-yl)carbamoyl)-5-(2,6-dimethoxyphenyl)-1H-pyrazol-1-yl)-3-isopropylphenyl)piperazine-2-carboxylic acid (70 mg, 1 Eq, 85 μmol) (from Example 48, Step 6) was treated with tert-butyl (tert-butoxycarbonyl)-L-lysinate (38 mg, 1.5 Eq, 0.13 mmol), HATU and DIEA in a manner similar to Example 154, Step 2, treated with TFA in a manner similar Example 1, Step 8, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((1-carboxy-5-((R)-4-(4-(3-(((1R,3 S,5R,7R)-2-carboxyadamantan-2-yl)carbamoyl)-5-(2,6-dimethoxyphenyl)-1H-pyrazol-1-yl)-3-isopropylphenyl)piperazine-2-carboxamido)pentyl)amino)-2-oxoethyl)-1,4,7,10-tetraazacyclododecane-1,4,7-triyl)triacetic acid (16.0 mg, 12.3 μmol, 28%) as a white solid. MS: Calc'd for C₈₉H₈₃N₁₁O₁₅: 1185.61, found [M+H]⁺: 1186.4.

### Example 157: Compound 157

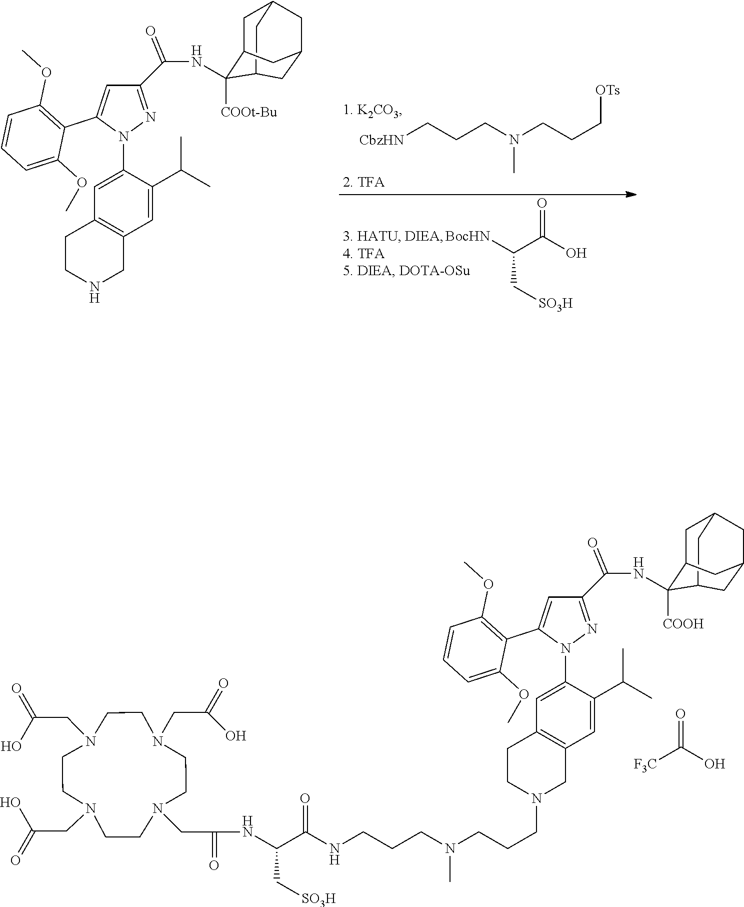

A mixture of tert-butyl (1R,3S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate (1.5 mg, 1 Eq, 160 μmol), K₂CO₃ (23 mg, 1.0 Eq, 0.17 mmol), 3-((3-(((benzyloxy)carbonyl)amino)propyl)(methyl)amino)propyl 4-methylbenzenesulfonate (209 mg, 3.00 Eq, 481 μmol) and DMF (2 mL) was stirred at 80° C. for 2 hours. The mixture was directly purified by MPLC to afford tert-butyl (1R,3S,5r,7r)-2-(1-(2-(3-((3-(((benzyloxy)carbonyl)amino)propyl)(methyl)amino)propyl)-7-isopropyl-1,2,3,4-tetrahydroisoquinolin-6-yl)-5-(2,6-dimethoxyphenyl)-1H-pyrazole-3-carboxamido)adamantane-2-carboxylate (70 mg, 76 μmol, 48%) as a yellow oil. [M+H]⁺=917.6.

A mixture of tert-butyl (1R,3 S,5r,7r)-2-(1-(2-(3-((3-(((benzyloxy)carbonyl)amino)propyl)(methyl)amino)propyl)-7-isopropyl-1,2,3,4-tetrahydroisoquinolin-6-yl)-5-(2,6-dimethoxyphenyl)-1H-pyrazole-3-carboxamido)adamantane-2-carboxylate (70 mg, 1 Eq, 76 μmol) and TFA (2 mL) was stirred at 70° C. for 1 hour. The mixture was concentrated under reduced pressure to afford (1R,3S,5r,7r)-2-(1-(2-(3-((3-aminopropyl)(methyl)amino)propyl)-7-isopropyl-1,2,3,4-tetrahydroisoquinolin-6-yl)-5-(2,6-dimethoxyphenyl)-1H-pyrazole-3-carboxamido)adamantane-2-carboxylic acid (70 mg, 96 μmol, 130%) as a yellow oil.

Steps 3-5: (1R,3S,5r,7r)-2-(1-(2-(3-((3-aminopropyl)(methyl)amino)propyl)-7-isopropyl-1,2,3,4-tetrahydroisoquinolin-6-yl)-5-(2,6-dimethoxyphenyl)-1H-pyrazole-3-carboxamido)adamantane-2-carboxylic acid was treated with (tert-butoxycarbonyl)(sulfo)-D-alanine (52 mg, 2.0 Eq, 0.19 mmol), HATU and DIEA in a manner similar to Example 154, Step 2, treated with TFA in a manner similar Example 1, Step 8, then treated with DIEA and 2,2′,2-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-(((R)-1-((3-((3-(6-(3-(((1R,3S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(11H)-yl)propyl)(methyl)amino)propyl)amino)-1-oxo-3-sulfopropan-2-yl)amino)-2-oxoethyl)-1,4,7,10-tetraazacyclododecane-1,4,7-triyl)triacetic acid (32.3 mg, 23.4 mol, 51%) as a light yellow solid. MS: Calc'd for C₆₁H₈₉N₁₁O₁₆S: 1263.62, found [M+H]⁺: 1264.6.

### Example 158: Compound 158

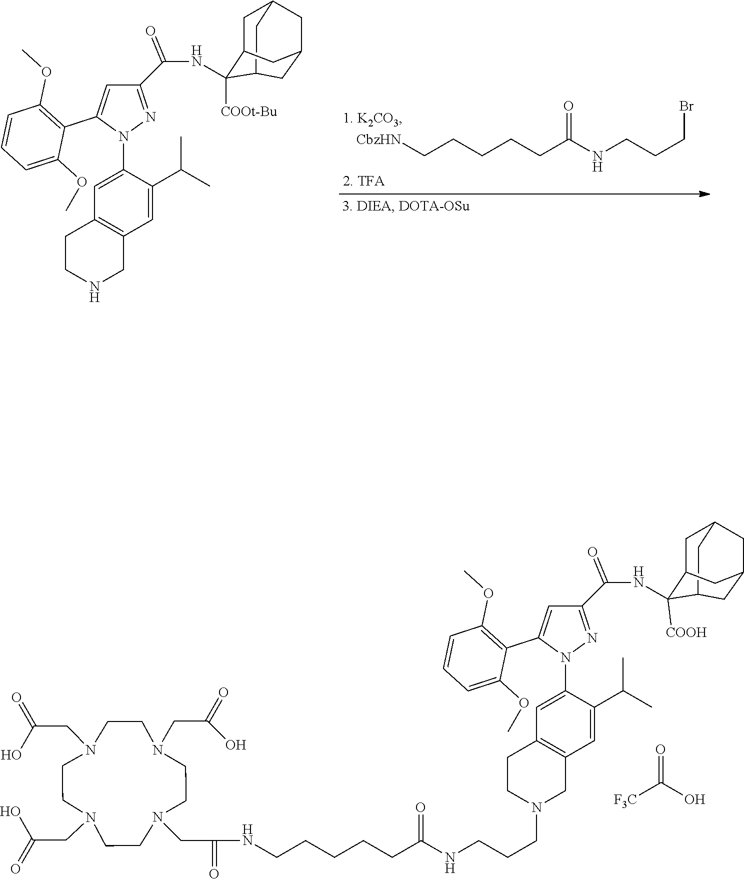

tert-Butyl (1R,3S,5r,7r)-2-(5-(2,6-dimethoxy phenyl)-1-(7-isopropyl-1,2,3,4-tetrahydroisoquinolin-6-yl)-1H-pyrazole-3-carboxamido)adamantane-2-carboxylate (60 mg, 1 Eq, 92 μmol), was treated with benzyl (6-((3-bromopropyl)amino)-6-oxohexyl)carbamate (180 mug, 5.1 Eq, 467 mol) and K₂CO₃, in manner similar to Example 113, Step 3, then treated with TFA in a manner similar to Example 8, Step 6, then treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((6-((3-(6-(3-(((1R,3 S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(1H)-yl)propyl)amino)-6-oxohexyl)amino)-2-oxoethyl)-1,4,7,10-tetraazacyclododecane-1,4,7-triyl)triacetic acid (8.1 mg, 6.4 μmol, 20%) as a white solid. MS: Calc'd for C₆₀H₈₆N₁₀O₁₃: 1154.64, found [M+H]⁺: 1155.6

### Example 159: Compound 159

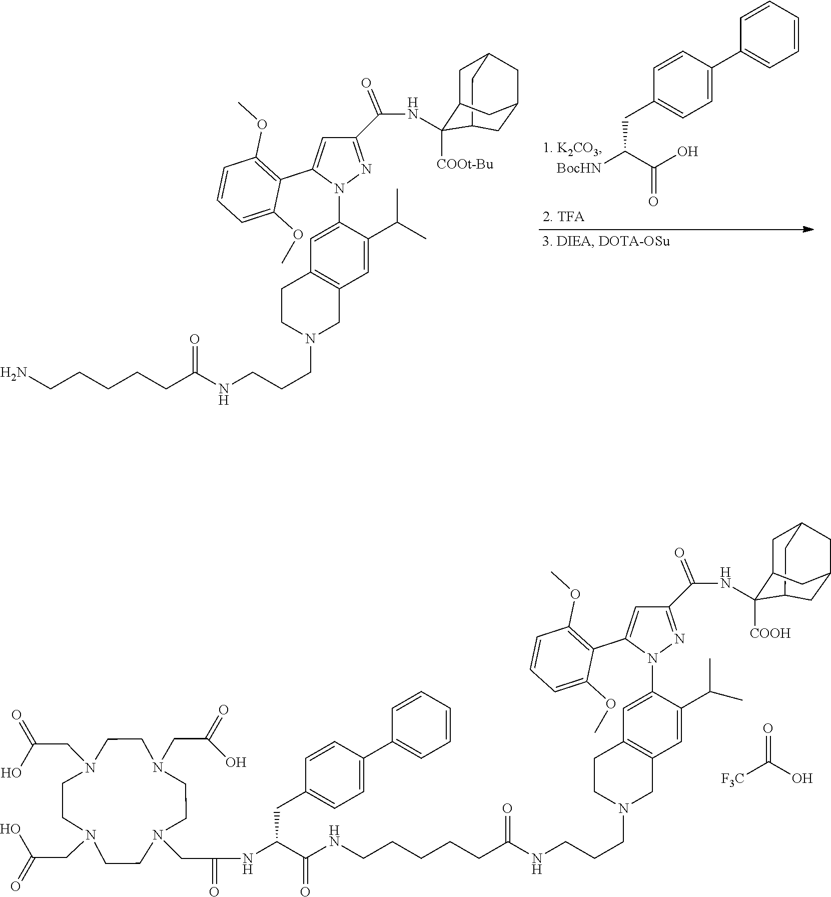

(1R,3S,5r,7r)-2-(1-(2-(3-(6-aminohexanamido)propyl)-7-isopropyl-1,2,3,4-tetrahydroisoquinolin-6-yl)-5-(2,6-dimethoxy phenyl)-1H-pyrazole-3-carboxamido)adamantane-2-carboxylic acid (45 mg, 90% Wt, 1 Eq, 53 μmol) (from Example 158, Step 2) was treated with (R)-3-([1,1′-biphenyl]-4-yl)-2-((tert-butoxycarbonyl)amino)propanoic acid (53 mg, 2.9 Eq, 0.16 mmol), HATU and DIEA in a manner similar to Example 154, Step 2, treated with TFA in a manner similar Example 1, Step 8, then treated with DIEA and 2,2′,2′-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-(((R)-3-([1,1′-biphenyl]-4-yl)-1-((6-((3-(6-(3-(((1R,3 S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(1H)-yl)propyl)amino)-6-oxohexyl)amino)-1-oxopropan-2-yl)amino)-2-oxoethyl)-1,4,7,10-tetraazacyclododecane-1,4,7-triyl)triacetic acid (10.9 mg, 7.30 μmol, 24%) as a white solid. MS: Calc'd for C₇₅H₉₉N₁₀O₁₄: 1377.74, found [M+H]⁺: 1378.6.

### Example 160: Compound 1.60

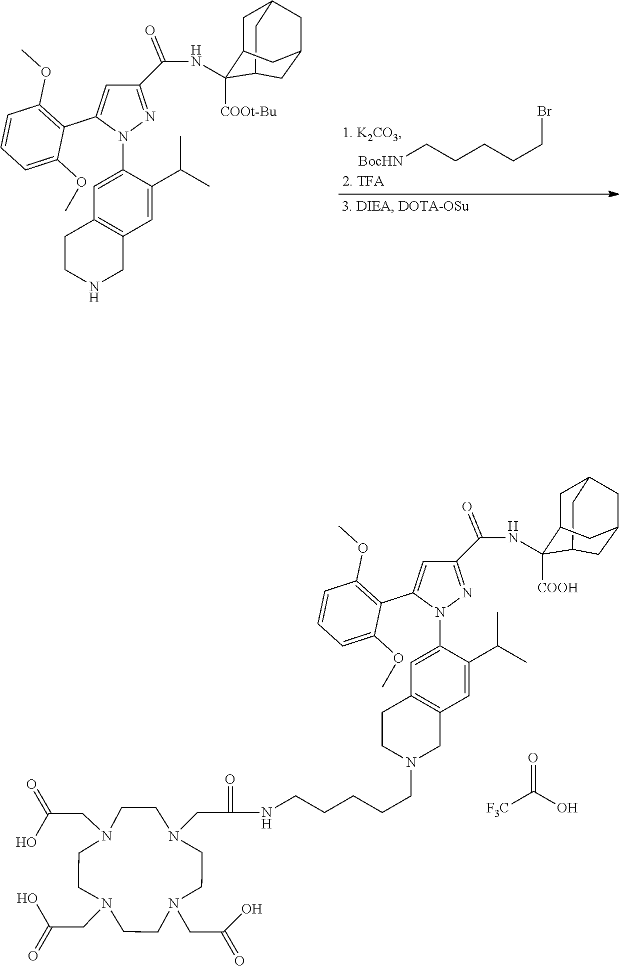

tert-Butyl (1R,3S,5r,7r)-2-(5-(2,6-dimethoxyphenyl)-1-(7-isopropyl-1,2,3,4-tetrahydroisoquinolin-6-yl)-1H-pyrazole-3-carboxamido)adamantane-2-carboxylate (60 mg, 1 Eq, 92 μmol) was treated with K₂CO₃and tert-butyl (5-bromopentyl)carbamate in a manner similar to Example 58, Step 2, treated with TFA in a manner similar Example 1, Step 8, then treated with DIEA and 2 2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 13, Step 5 to afford the 2,2,2-trifluoroacetic acid salt of 2,2′,2″-(10-(2-((5-(6-(3-(((1R,3S,5r,7r)-2-carboxyadamantan-2-yl)carbamoyl)-5-(2,6-dimethoxyphenyl)-1H-pyrazol-1-yl)-7-isopropyl-3,4-dihydroisoquinolin-2(1H)-yl)pentyl)amino)-2-oxoethyl)-1,4,7,10-tetraazacyclododecane-1,4,7-triyl)triacetic acid (32.3 mg, 27.3 μmol, 53%) as a white solid. MS: Calc'd for C₅₆H₇₉N₉O₁₂: 1069.58, found [M+H]⁺: 1070.6

### Example A-1: Parenteral Pharmaceutical Composition

To prepare a parenteral pharmaceutical composition suitable for administration by injection (subcutaneous, intravenous), 0.001-500 mg of a compound Formula (A) or Formula (I), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Biology Examples

### Example B-1: Human NTSR1 Binding Assays

### Membrane Preparation

Crude membrane fractions are prepared from HT-29 (ATCC) cells endogenously expressing the NTSR1 receptor. The cells are grown to 85-100% confluence on standard tissue culture dishes in McCoy's 5a Medium (ATCC) supplemented with 10% FBS (Gemini). To prepare membranes, cells are scraped and collected in 1× Dulbecco's phosphate buffered saline (Corning) and then pelleted at 1000 RPM's. The cell pellet is reconstituted in membrane preparation buffer (20 mM HEPES, 6 mM MgCl₂ and 1 mM EGTA, protease inhibitor tablets (Pierce), pH 7.4) and placed in a cell disruption vessel (Parr Instrument company) at 1000 PSI for 30 minutes on ice. The pressurized contents are then released and spun down at 1000 RPMs and the supernatant is collected and further centrifuged at 15,000 RPMs to pellet the membranes. The membrane pellet is resuspended in membrane preparation buffer, snap freezed and stored at −80° C. for later use.

### Human NTSR1 Binding Assay Protocol—120 Minute Incubation:

The NTSR1 membrane binding assay utilizes the following components: radiolabel [1251]-Tyr3-Neurotensin (human) (Revvity), crude HT-29 membranes, and competing small molecule and peptide ligands. The assay is initiated by combining in assay buffer (50 mM HEPES, 5 mM MgCl₂, 1 mM CaCl₂, 0.2% BSA, pH7.4) a dose response of competing ligand (the final concentrations are typically 0-10,000 nM), HT-29 membranes, and [125I] Tyr3-Neurotensin (human) at a final concentration of 0.1 nM in a 96-well assay plate and allowed to incubate 120 minutes at room temperature. Assay contents are then filtered through unifilter GF/C microplates (Revvity) and washed with 4×400 μl of cold wash buffer (50 mM HEPES, 500 mM NaCl, 0.1% BSA, pH7.4). Assay plates are read using a MicroBeta² (Revvity) and Ki values for compounds are determined using a GraphPad Prism 10 non-linear regression analysis. Human NTSR1 Binding Assay—60 minute incubation: The NTSR1 membrane binding assay is a competition binding assay using radiolabel [125I]-Tyr3-Neurotensin (human) at a final concentration of 0.05 nM, crude Cho membranes recombinantly expressing the human NTSR1 receptor, and competing small molecule and peptide ligands (the final concentrations 0-10,000 nM). The assay is incubated for 60 minutes at 4° C.

Illustrative biological activity of compounds is demonstrated in Table E. Most reported binding Ki's were measured using the method employing a 120 minute incubation period, however, the binding Ki's marked with the “\hat{ }” symbol were measured using the method employing a 60 minute incubation period. The metal complexes in Table E comprise nonradioactive gallium, indium, and lutetium

TABLE E
Representative Binding Activity

| | hNTSR1 |
|---|---|
| | Binding |
| Cmpnd | Ki* (nM) |
| 1 | A\hat{ } |
| 2 | A\hat{ } |
| 3 | A |
| 4 | A |
| 5 | A |
| 6 | A\hat{ } |
| 8 | B |
| 9 | C |
| 10 | A |
| 11 | A\hat{ } |
| 12 | A\hat{ } |
| 13 | A |
| 14 | A |
| 15 | A |
| 15-Lu | A |
| 15-Ga | A |
| 16 | A |
| 17 | A |
| 18 | A |
| 19 | A |
| 20 | A |
| 21 | A |
| 22 | A |
| 23 | A |
| 24 | A |
| 24-Lu | A |
| 25 | A |
| 26 | A |
| 27 | A |
| 28 | A |
| 29 | A |
| 30 | A |
| 31 | A |
| 32 | A |
| 32-Lu | A |
| 33 | A |
| 34 | A |
| 35 | A |
| 36 | A |
| 37 | A |
| 38 | A |
| 41 | A |
| 43 | A |
| 44 | A |
| 45 | A |
| 46 | A |
| 47 | A |
| 48 | A |
| 50 | A |
| 51 | A |
| 52 | A |
| 53 | A |
| 54 | A |
| 55 | A |
| 55-Lu | A |
| 56 | A |
| 57 | A |
| 57-Lu | A |
| 58 | A |
| 59 | A |
| 60 | A |
| 64 | C |
| 65 | C |
| 66 | A |
| 67 | A |
| 68 | A |
| 69 | A |
| 70 | A |
| 71 | A |
| 72 | A |
| 73 | A |
| 74 | A |
| 76 | A |
| 78 | A |
| 79 | B |
| 80 | A |
| 81 | A |
| 83 | A |
| 84 | A |
| 85 | A |
| 86 | A |
| 87 | A |
| 88 | A |
| 91 | A |
| 92 | A |
| 93 | A |
| 94 | A |
| 95 | A |
| 96 | A |
| 97 | A |
| 98 | A |
| 99 | A |
| 99-Lu | A |
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | A |
| 116 | A |
| 116-Lu | A |
| 116-Ga | A |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | A |
| 131 | A |
| 132 | B |
| 133 | A |
| 134 | A |
| 135A | A |
| 135B | A |
| 136 | A |
| 137 | A |
| 138 | A |
| 139 | A |
| 140 | A |
| 141 | A |
| 142 | A |
| 142-Lu | A |
| 142-Ga | A |
| 143 | A |
| 144 | A |
| 145 | A |
| 146 | A |
| 147 | A |
| 148 | A |
| 149 | A |
| 150 | C |
| 151 | A |
| 152 | A |
| 153 | A |
| 154 | A |
| 155 | A |
| 156 | A |
| 157 | A |
| 158 | A |
| 159 | A |
| 160 | A |

*A is <10 nM; B is 10-100 nM; C is >100 nM

### Example B-2: General Protocol to Measure Biodistribution of ¹⁷⁷Lu[Lu] Complexes of Lead Compounds in Mouse Tumor Model

Twenty-four hours prior to the start of the biodistribution, the compound was radiolabeled.

Prior to initiation of the biodistribution study, female Swiss nude mice were subcutaneously inoculated with 10×10⁶ HT-29 human colon cancer cells.

When tumors were between 150-300 mm³, animals were randomized and received a single IV injection of 200 uL into the caudal vein via a catheter with 5-7 MBq of ¹⁷⁷Lu[Lu] Compound X (1 nMol) per animal. For the competition study arm, 1 nMol of ¹⁷⁷Lu[Lu]-Compound X was co injected with 100 nMol of ¹⁷⁵Lu[Lu]-Compound X.

After drug administration, animals were euthanized at timepoints (0.5 h, 2 h, 5 h, 24 h, 72 h and 168 h) and organs (blood, tumor, heart, kidneys, adrenals, liver (with gallbladder), pancreas, spleen, lungs, stomach, small intestine, large intestine (with caecum), bone (femur), muscle, brain, carcass and tail) were collected, weighed, and ÿ radioactivity assessed in each organ/tissue. Activity was quantitated and expressed as % ID/g (Percentage of Initial Dose/gram of tissue).

### Example B-3: Efficacy of ¹⁷⁷Lu[Lu] Complexes of Compound 1-7, Compound 15, Compound 116, and Compound 142 in Mouse Tumor Model Methods

Tumor cells were inoculated subcutaneously in the right rear flank by injection of 10 million (1×10⁷) HT-29 cells in 200 μL of RPMI 1640 (without phenol red) of 91 female Swiss nude mice. HT-29 tumor cell implantation was performed 24 to 72 hours after a whole-body irradiation with a gamma-source (2 Gy (Nude mice)).

Once tumors reached a mean value of approximately 225 mm³ (day 12), animals were randomized into 5 groups (9 animals per group) and dosed with vehicle, 45 MBq of 177Lu-radiolabeled complexes (at 0.4 total nmol of each compound for 4 compounds). Tumors were measured twice weekly and tumor volumes were determined by the formula: Tumor Volume=(width\hat{ }(2)×length)/2. Mouse mass was calculated 2-3 times per week. Tumor growth inhibition (TGI mean %) was calculated as follows: TGI (%)=(1-(mean TV on treated group)/(mean TV on control group))×100. TGI % was calculated up to the last day on which the number of animals in the vehicle group (control) exceeds 50%.

Structure of Compound I-7 (I-7 is disclosed in PCT Publication WO2023158802):

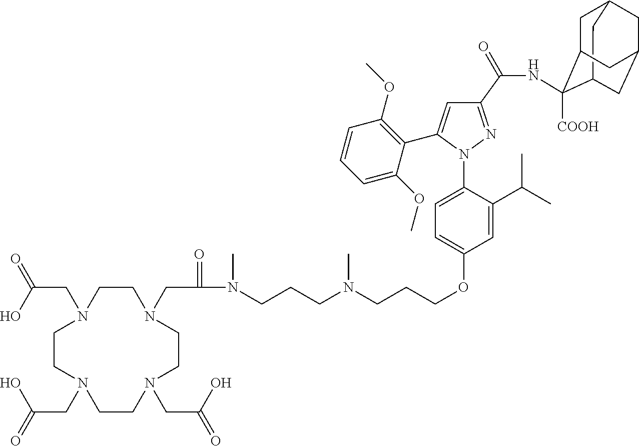 [SMILES: COc1cccc(OC)c1-c1cc(C(=O)NC2(C(=O)O)C3CC4CC(C3)CC2C4)nn1-c1ccc(OCCCN(C)CCCN(C)C(=O)CN2CCN(CC(=O)O)CCN(CC(=O)O)CCN(CC(=O)O)CC2)cc1C(C)C]

TABLE F
Tumor growth inhibition (TGI) of ¹⁷⁷Lu[Lu] complexes of
Compound I-7, Compound 15, Compound 116, and Compound 142
at Day 32 and Day 35

| Group | TGI Day 32 | TGI Day 35 |
|---|---|---|
| Vehicle | N/A | N/A |
| ¹⁷⁷Lu[Lu]-Compound 15-45 MBq | 83% | 85% |
| ¹⁷⁷Lu[Lu]-Compound I-7-45 MBq | 51% | 53% |
| ¹⁷⁷Lu[Lu]-Compound 142-45 MBq | 78% | 82% |
| ¹⁷⁷Lu-[Lu]-Compound 116-45 MBq | 25% | 21% |

### Example B-4: Safety and Biodistribution Study in Patients with Colorectal Cancer, Pancreatic Cancer, and Head and/or Neck Cancer

A non-limiting example of a phase I safety and dosimetry study of a ⁶⁸Ga and/or ¹⁷⁷Lu-complex of a compound of Formula (I) or Formula (A) in patients with colorectal, pancreatic, and/or head and neck cancer is described below.

This is an open-label, first-in-human, Phase I study of a ⁶⁸Ca and/or ¹⁷⁷Lu-complex of a compound of Formula (I) or Formula (A) designed to characterize safety and biodistribution in patients diagnosed with colorectal, pancreatic, and head and/or neck cancer. In some embodiments, a ⁶⁸Ga or ¹⁷⁷Lu-complex of a compound of Formula (I) or Formula (A) is used to localize NTSR1-expressing lesions and identify colorectal, pancreatic, and head and/or neck cancer patients with NTSR1-expressing tumors who may benefit from treatment with NTSR1-targeting therapeutic agents, such as a ¹⁷⁷Lu-complex of a compound of Formula (I) or Formula (A). In healthy humans, NTSR1 is found primarily in the hypothalamus, pituitary and placenta, and plays a role in regulating puberty and fertility. NTSR1 is expressed in colorectal, pancreatic and head and neck cancer.

This study will enroll approximately 21 evaluable subjects in total. Patients with metastatic colorectal cancer, pancreatic cancer, and/or head and neck cancer are eligible to participate if they meet all inclusion criteria and none of the exclusion criteria.

Study Populations: Patients with metastatic colorectal, pancreatic and head and/or neck cancer.

Primary Objectives: To describe the safety of a ⁶⁸Ga and/or ¹⁷⁷Lu-complex of a compound of Formula (I) or Formula (A).

Secondary Objectives: To describe the biodistribution of a ⁶⁸Ga and/or ¹⁷⁷Lu-complex of a compound of Formula (I) or Formula (A), to compare Formula (I) or Formula (A) positron emission tomography/computed tomography (PET/CT) and single emission photon tomography/computed tomography (SPECT/CT) scans to standard of care anatomic imaging in detecting tumor lesions.

Primary Endpoints: Incidence of adverse events (AE) characterized overall and by type, frequency, seriousness, relationship to the study drug, timing, and severity, graded according to the National Cancer Institute (NCI) Common Terminology Criteria for Adverse Events (CTCAE), Version 5.0

Secondary Endpoints: Maximum standard uptake value (SUVₘₐₓ) of each tumor and SUVₘₑₐₙ of organs. Ratio of the tumor SUV over reference region SUV. Number and location of tumors identified by a ⁶⁸Ga and ¹⁷⁷Lu-complex of a compound of Formula (I) or Formula (A) on PET/CT or SPECT/CT, and concordance rate between ⁶⁸Ga and ¹⁷⁷Lu-complex of a compound of Formula (I) PET/CT and standard of care images.

Study Design: This study will enroll up to approximately 21 evaluable patients. Patients who receive study drug and complete all scheduled imaging procedures are considered evaluable.

There are three patient cohorts in the study: Cohort 1: patients with metastatic colorectal cancer; Cohort 2: patients with metastatic pancreatic cancer; Cohort 3. patients with metastatic head and neck cancer. Patients are enrolled into the three cohorts in parallel. Each cohort will enroll approximately 7 evaluable patients.

Each eligible patient enrolled will receive a single intravenous injection of a ⁶⁸Ga-complex of a compound of Formula (I) or Formula (A) on Day 1 of the study at an activity of approximately 5 mCi (185 MBq). Some patients may receive low-dose ¹⁷⁷Lu-complex of a compound of Formula (I) or Formula (A). Patients will be imaged with PET/CT following ⁶⁸Ga-complex administration and with SPECT/CT after ¹⁷⁷Lu-complex administration at the timepoints specified in the protocol.

All patients must meet all the inclusion eligibility criteria and none of the exclusion eligibility criteria, as appropriate and provided below.

Inclusion Criteria: Metastatic colorectal cancer (Cohort 1 patients); metastatic pancreatic cancer (Cohort 2 patients); locally advanced or metastatic head and neck cancer (Cohort 3 patients). Male or non-pregnant, non-lactating female subjects age≥18 years. Subjects who are sexually active must agree to use adequate method(s) of effective contraception during their participation in the study. Eastern Cooperative Oncology Group (ECOG) Performance Status≤2. Adequate hepatic function as defined by a) serum alanine aminotransaminase (ALT)/aspartate aminotransaminase (AST)≤3×upper limit of normal (ULN) or ≤5×ULN if liver metastases are present or received prior mitotane therapy, and b) serum bilirubin−total≤1.5×ULN (unless due to Gilbert's syndrome or hemolysis in which case total≤3.0×ULN). Adequate renal function as measured by creatinine clearance calculated by the Cockcroft-Gault formula (≥60 mL/minute). Able to understand and willing to sign written informed consent.

Exclusion Criteria: Administered a radionuclide within a period of time corresponding to less than 10 physical half-lives of the radionuclide prior to study Day 1. Radiotherapy≤14 days prior to study Day 1. Major surgery≤21 days prior to study Day 1 or has not recovered from adverse effects of such procedure. History of cerebrovascular accident within 6 months or that resulted in ongoing neurologic instability. History of other previous or concurrent cancer that would interfere with the determination of safety. Any other condition that in the opinion of the Investigator would place the subject at an unacceptable risk or cause the subject to be unlikely to fully participate or comply with study procedures.

### Study Drug, Dose, and Mode of Administration

Study Drug: The study drug is a ⁶⁸Ga-complex or ¹⁷⁷Lu-complex of a compound of Formula (I) or Formula (A). In some embodiments, the ⁶⁸Ga-complex is a ⁶⁸Ga-complex of a compound of Formula (I) or Formula (A). In some embodiments, the ¹⁷⁷Lu-complex is a ¹⁷⁷Lu-complex of a compound of Formula (I) or Formula (A). In some embodiments, the ⁶⁸Ga-complex is a ⁶⁸Ga-complex of Compound 13. In some embodiments, the ⁶⁸Ga-complex is a ⁶⁸Ga-complex of Compound 14. In some embodiments, the ⁶⁸Ga-complex is a ⁶⁸Ga-complex of Compound 15. In some embodiments, the ⁶⁸Ga-complex is a ⁶⁸Ga-complex of Compound 16. In some embodiments, the ⁶⁸Ga-complex is a ⁶⁸Ga-complex of Compound 17. In some embodiments, the ⁶⁸Ga-complex is a ⁶⁸Ga-complex of Compound 18. In some embodiments, the ⁶⁸Ga-complex is a ⁶⁸Ga-complex of Compound 19. In some embodiments, the ⁶⁸Ga-complex is a ⁶⁸Ga-complex of Compound 20. In some embodiments, the ⁶⁸Ga-complex is a ⁶⁸Ga-complex of Compound 21. In some embodiments, the ⁶⁸Ga-complex is a ⁶⁸Ga-complex of Compound 22. In some embodiments, the ⁶⁸Ga-complex is a ⁶⁸Ga-complex of Compound 23. In some embodiments, the ⁶⁸Ga-complex is a ⁶⁸Ga-complex of Compound 24. In some embodiments, the ⁶⁸Ga-complex is a ⁶⁸Ga-complex of Compound 25. In some embodiments, the ⁶⁸Ga-complex is a ⁶⁸Ga-complex of Compound 26. In some embodiments, the ⁶⁸Ga-complex is a ⁶⁸Ga-complex of Compound 27. In some embodiments, the ⁶⁸Ga-complex is a ⁶⁸Ga-complex of Compound 28. In some embodiments, the ⁶⁸Ga-complex is a ⁶⁸Ga-complex of Compound 29. In some embodiments, the ⁶⁸Ga-complex is a ⁶⁸Ga-complex of Compound 30. In some embodiments, the ⁶⁸Ga-complex is a ⁶⁸Ga-complex of Compound 31. In some embodiments, the ⁶⁸Ga-complex is a ⁶⁸Ga-complex of Compound 32. In some embodiments, the ⁶⁸Ga-complex is a ⁶⁸Ga-complex of Compound 33. In some embodiments, the ⁶⁸Ga-complex is a ⁶⁸Ga-complex of Compound 34. In some embodiments, the ⁶⁸Ga-complex is a ⁶⁸Ga-complex of Compound 35. In some embodiments, the ⁶⁸Ga-complex is a ⁶⁸Ga-complex of Compound 36. In some embodiments, the ⁶⁸Ga-complex is a ⁶⁸Ga-complex of Compound 37. In some embodiments, the ⁶⁸Ga-complex is a ⁶⁸Ga-complex of Compound 38. In some embodiments, the ⁶⁸Ga-complex is a ⁶⁸Ga-complex of Compound 39. In some embodiments, the ⁶⁸Ga-complex is a ⁶⁸Ga-complex of Compound 40. In some embodiments, the ⁶⁸Ga-complex is a ⁶⁸Ga-complex of Compound 41. In some embodiments, the ⁶⁸Ga-complex is a ⁶⁸Ga-complex of Compound 42. In some embodiments, the ⁶⁸Ga-complex is a ⁶⁸Ga-complex of Compound 43. In some embodiments, the ⁶⁸Ga-complex is a ⁶⁸Ga-complex of Compound 44. In some embodiments, the ⁶⁸Ga-complex is a ⁶⁸Ga-complex of Compound 45. In some embodiments, the ⁶⁸Ga-complex is a ⁶⁸Ga-complex of Compound 46. In some embodiments, the ⁶⁸Ga-complex is a ⁶⁸Ga-complex of Compound 47. In some embodiments, the ⁶⁸Ga-complex is a ⁶⁸Ga-complex of Compound 48. In some embodiments, the ⁶⁸Ga-complex is a ⁶⁸Ga-complex of Compound 49. In some embodiments, the ⁶⁸Ga-complex is a ⁶⁸Ga-complex of Compound 50. In some embodiments, the ⁶⁸Ga-complex is a ⁶⁸Ga-complex of Compound 51. In some embodiments, the ⁶⁸Ga-complex is a ⁶⁸Ga-complex of Compound 52. In some embodiments, the ⁶⁸Ga-complex is a ⁶⁸Ga-complex of Compound 53. In some embodiments, the ⁶⁸Ga-complex is a ⁶⁸Ga-complex of Compound 54. In some embodiments, the ⁶⁸Ga-complex is a ⁶⁸Ga-complex of Compound 55. In some embodiments, the ⁶⁸Ga-complex is a ⁶⁸Ga-complex of Compound 56. In some embodiments, the ⁶⁸Ga-complex is a ⁶⁸Ga-complex of Compound 57. In some embodiments, the ⁶⁸Ga-complex is a ⁶⁸Ga-complex of Compound 58. In some embodiments, the ⁶⁸Ga-complex is a ⁶⁸Ga-complex of Compound 59. In some embodiments, the ⁶⁸Ga-complex is a ⁶⁸Ga-complex of Compound 60. In some embodiments, the ⁶⁸Ga-complex is a ⁶⁸Ga-complex of Compound 61. In some embodiments, the ⁶⁸Ga-complex is a ⁶⁸Ga-complex of Compound 62. In some embodiments, the ⁶⁸Ga-complex is a ⁶⁸Ga-complex of Compound 63. In some embodiments, the ⁶⁸Ga-complex is a ⁶⁸Ga-complex of Compound 64. In some embodiments, the ⁶⁸Ga-complex is a ⁶⁸Ga-complex of Compound 65. In some embodiments, the ⁶⁸Ga-complex is a ⁶⁸Ga-complex of Compound 66. In some embodiments, the ⁶⁸Ga-complex is a ⁶⁸Ga-complex of Compound 67. In some embodiments, the ⁶⁸Ga-complex is a ⁶⁸Ga-complex of Compound 68. In some embodiments, the ⁶⁸Ga-complex is a ⁶⁸Ga-complex of Compound 69. In some embodiments, the ⁶⁸Ga-complex is a ⁶⁸Ga-complex of Compound 70. In some embodiments, the ⁶⁸Ga-complex is a ⁶⁸Ga-complex of Compound 71. In some embodiments, the ⁶⁸Ga-complex is a ⁶⁸Ga-complex of Compound 72. In some embodiments, the ⁶⁸Ga-complex is a ⁶⁸Ga-complex of Compound 73. In some embodiments, the ⁶⁸Ga-complex is a ⁶⁸Ga-complex of Compound 74. In some embodiments, the ⁶⁸Ga-complex is a ⁶⁸Ga-complex of Compound 75. In some embodiments, the ⁶⁸Ga-complex is a ⁶⁸Ga-complex of Compound 84. In some embodiments, the ⁶⁸Ga-complex is a ⁶⁸Ga-complex of Compound 85. In some embodiments, the ⁶⁸Ga-complex is a ⁶⁸Ga-complex of Compound 86. In some embodiments, the ⁶⁸Ga-complex is a ⁶⁸Ga-complex of Compound 87. In some embodiments, the ⁶⁸Ga-complex is a ⁶⁸Ga-complex of Compound 88. In some embodiments, the ⁶⁸Ga-complex is a ⁶⁸Ga-complex of Compound 89. In some embodiments, the ⁶⁸Ga-complex is a ⁶⁸Ga-complex of Compound 90. In some embodiments, the ⁶⁸Ga-complex is a ⁶⁸Ga-complex of Compound 91. In some embodiments, the ⁶⁸Ga-complex is a ⁶⁸Ga-complex of Compound 92. In some embodiments, the ⁶⁸Ga-complex is a ⁶⁸Ga-complex of Compound 93. In some embodiments, the ⁶⁸Ga-complex is a ⁶⁸Ga-complex of Compound 94. In some embodiments, the ⁶⁸Ga-complex is a ⁶⁸Ga-complex of Compound 95. In some embodiments, the ⁶⁸Ga-complex is a ⁶⁸Ga-complex of Compound 96. In some embodiments, the ⁶⁸Ga-complex is a ⁶⁸Ga-complex of Compound 97. In some embodiments, the ⁶⁸Ga-complex is a ⁶⁸Ga-complex of Compound 98. In some embodiments, the ⁶⁸Ga-complex is a ⁶⁸Ga-complex of Compound 99. In some embodiments, the ⁶⁸Ga-complex is a ⁶⁸Ga-complex of Compound 100. In some embodiments, the ⁶⁸Ga-complex is a ⁶⁸Ga-complex of Compound 101. In some embodiments, the ⁶⁸Ga-complex is a ⁶⁸Ga-complex of Compound 102. In some embodiments, the ⁶⁸Ga-complex is a ⁶⁸Ga-complex of Compound 103. In some embodiments, the ⁶⁸Ga-complex is a ⁶⁸Ga-complex of Compound 104. In some embodiments, the ⁶⁸Ga-complex is a ⁶⁸Ga-complex of Compound 105. In some embodiments, the ⁶⁸Ga-complex is a ⁶⁸Ga-complex of Compound 106. In some embodiments, the ⁶⁸Ga-complex is a ⁶⁸Ga-complex of Compound 107. In some embodiments, the ⁶⁸Ga-complex is a ⁶⁸Ga-complex of Compound 108. In some embodiments, the ⁶⁸Ga-complex is a ⁶⁸Ga-complex of Compound 109. In some embodiments, the ⁶⁸Ga-complex is a ⁶⁸Ga-complex of Compound 110. In some embodiments, the ⁶⁸Ga-complex is a ⁶⁸Ga-complex of Compound 111. In some embodiments, the ⁶⁸Ga-complex is a ⁶⁸Ga-complex of Compound 112. In some embodiments, the ⁶⁸Ga-complex is a ⁶⁸Ga-complex of Compound 113. In some embodiments, the ⁶⁸Ga-complex is a ⁶⁸Ga-complex of Compound 114. In some embodiments, the ⁶⁸Ga-complex is a ⁶⁸Ga-complex of Compound 115. In some embodiments, the ⁶⁸Ga-complex is a ⁶⁸Ga-complex of Compound 116. In some embodiments, the ⁶⁸Ga-complex is a ⁶⁸Ga-complex of Compound 117. In some embodiments, the ⁶⁸Ga-complex is a ⁶⁸Ga-complex of Compound 118. In some embodiments, the ⁶⁸Ga-complex is a ⁶⁸Ga-complex of Compound 119. In some embodiments, the ⁶⁸Ga-complex is a ⁶⁸Ga-complex of Compound 120. In some embodiments, the ⁶⁸Ga-complex is a ⁶⁸Ga-complex of Compound 121. In some embodiments, the ⁶⁸Ga-complex is a ⁶⁸Ga-complex of Compound 122. In some embodiments, the ⁶⁸Ga-complex is a ⁶⁸Ga-complex of Compound 123. In some embodiments, the ⁶⁸Ga-complex is a ⁶⁸Ga-complex of Compound 124. In some embodiments, the ⁶⁸Ga-complex is a ⁶⁸Ga-complex of Compound 137. In some embodiments, the ⁶⁸Ga-complex is a ⁶⁸Ga-complex of Compound 138. In some embodiments, the ⁶⁸Ga-complex is a ⁶⁸Ga-complex of Compound 139. In some embodiments, the ⁶⁸Ga-complex is a ⁶⁸Ga-complex of Compound 140. In some embodiments, the ⁶⁸Ga-complex is a ⁶⁸Ga-complex of Compound 141. In some embodiments, the ⁶⁸Ga-complex is a ⁶⁸Ga-complex of Compound 142. In some embodiments, the ⁶⁸Ga-complex is a ⁶⁸Ga-complex of Compound 143. In some embodiments, the ⁶⁸Ga-complex is a ⁶⁸Ga-complex of Compound 144. In some embodiments, the ⁶⁸Ga-complex is a ⁶⁸Ga-complex of Compound 145. In some embodiments, the ⁶⁸Ga-complex is a ⁶⁸Ga-complex of Compound 146. In some embodiments, the ⁶⁸Ga-complex is a ⁶⁸Ga-complex of Compound 147. In some embodiments, the ⁶⁸Ga-complex is a ⁶⁸Ga-complex of Compound 148. In some embodiments, the ⁶⁸Ga-complex is a ⁶⁸Ga-complex of Compound 149. In some embodiments, the ⁶⁸Ga-complex is a ⁶⁸Ga-complex of Compound 150. In some embodiments, the ⁶⁸Ga-complex is a ⁶⁸Ga-complex of Compound 151. In some embodiments, the ⁶⁸Ga-complex is a ⁶⁸Ga-complex of Compound 152. In some embodiments, the ⁶⁸Ga-complex is a ⁶⁸Ga-complex of Compound 153. In some embodiments, the ⁶⁸Ga-complex is a ⁶⁸Ga-complex of Compound 154. In some embodiments, the ⁶⁸Ga-complex is a ⁶⁸Ga-complex of Compound 155. In some embodiments, the ⁶⁸Ga-complex is a ⁶⁸Ga-complex of Compound 156. In some embodiments, the ⁶⁸Ga-complex is a ⁶⁸Ga-complex of Compound 157. In some embodiments, the ⁶⁸Ga-complex is a ⁶⁸Ga-complex of Compound 158. In some embodiments, the ⁶⁸Ga-complex is a ⁶⁸Ga-complex of Compound 159. In some embodiments, the ⁶⁸Ga-complex is a ⁶⁸Ga-complex of Compound 160. In some embodiments, the ¹⁷⁷Lu-complex is a ¹⁷⁷Lu-complex of Compound 13. In some embodiments, the 177Lu-complex is a ¹⁷⁷Lu-complex of Compound 14. In some embodiments, the ¹⁷⁷Lu-complex is a ¹⁷⁷Lu-complex of Compound 15. In some embodiments, the 177Lu-complex is a ¹⁷⁷Lu-complex of Compound 16. In some embodiments, the ¹⁷⁷Lu-complex is a ¹⁷⁷Lu-complex of Compound 17. In some embodiments, the ¹⁷⁷Lu-complex is a ¹⁷⁷Lu-complex of Compound 18. In some embodiments, the ¹⁷⁷Lu-complex is a ¹⁷⁷Lu-complex of Compound 19. In some embodiments, the ¹⁷⁷Lu-complex is a ¹⁷⁷Lu-complex of Compound 20. In some embodiments, the ¹⁷⁷Lu-complex is a ¹⁷⁷Lu-complex of Compound 21. In some embodiments, the ¹⁷⁷Lu-complex is a ¹⁷⁷Lu-complex of Compound 22. In some embodiments, the ¹⁷⁷Lu-complex is a ¹⁷⁷Lu-complex of Compound 23. In some embodiments, the 177Lu-complex is a ¹⁷⁷Lu-complex of Compound 24. In some embodiments, the ¹⁷⁷Lu-complex is a ¹⁷⁷Lu-complex of Compound 25. In some embodiments, the 177Lu-complex is a ¹⁷⁷Lu-complex of Compound 26. In some embodiments, the ¹⁷⁷Lu-complex is a ¹⁷⁷Lu-complex of Compound 27. In some embodiments, the ¹⁷⁷Lu-complex is a ¹⁷⁷Lu-complex of Compound 28. In some embodiments, the 177Lu-complex is a 177Lu-complex of Compound 29. In some embodiments, the 177Lu-complex is a 177Lu-complex of Compound 30. In some embodiments, the 177Lu-complex is a 177Lu-complex of Compound 31. In some embodiments, the ¹⁷⁷Lu-complex is a 177Lu-complex of Compound 32. In some embodiments, the 177Lu-complex is a ¹⁷⁷Lu-complex of Compound 33. In some embodiments, the 177Lu-complex is a 177Lu-complex of Compound 34. In some embodiments, the ¹⁷⁷Lu-complex is a ¹⁷⁷Lu-complex of Compound 35. In some embodiments, the 177Lu-complex is a 177Lu-complex of Compound 36. In some embodiments, the 177Lu-complex is a 177Lu-complex of Compound 37. In some embodiments, the 177Lu-complex is a 177Lu-complex of Compound 38. In some embodiments, the ¹⁷⁷Lu-complex is a ¹⁷⁷Lu-complex of Compound 39. In some embodiments, the ¹⁷⁷Lu-complex is a ¹⁷⁷Lu-complex of Compound 40. In some embodiments, the ¹⁷⁷Lu-complex is a ¹⁷⁷Lu-complex of Compound 41. In some embodiments, the ¹⁷⁷Lu-complex is a ¹⁷⁷Lu-complex of Compound 42. In some embodiments, the ¹⁷⁷Lu-complex is a ¹⁷⁷Lu-complex of Compound 43. In some embodiments, the 177Lu-complex is a ¹⁷⁷Lu-complex of Compound 44. In some embodiments, the ¹⁷⁷Lu-complex is a ¹⁷⁷Lu-complex of Compound 45. In some embodiments, the 177Lu-complex is a ¹⁷⁷Lu-complex of Compound 46. In some embodiments, the ¹⁷⁷Lu-complex is a ¹⁷⁷Lu-complex of Compound 47. In some embodiments, the ¹⁷⁷Lu-complex is a ¹⁷⁷Lu-complex of Compound 48. In some embodiments, the ¹⁷⁷Lu-complex is a ¹⁷⁷Lu-complex of Compound 49. In some embodiments, the ¹⁷⁷Lu-complex is a ¹⁷⁷Lu-complex of Compound 50. In some embodiments, the ¹⁷⁷Lu-complex is a ¹⁷⁷Lu-complex of Compound 51. In some embodiments, the ¹⁷⁷Lu-complex is a ¹⁷⁷Lu-complex of Compound 52. In some embodiments, the ¹⁷⁷Lu-complex is a ¹⁷⁷Lu-complex of Compound 53. In some embodiments, the 177Lu-complex is a ¹⁷⁷Lu-complex of Compound 54. In some embodiments, the ¹⁷⁷Lu-complex is a ¹⁷⁷Lu-complex of Compound 55. In some embodiments, the ¹⁷⁷Lu-complex is a ¹⁷⁷Lu-complex of Compound 56. In some embodiments, the ¹⁷⁷Lu-complex is a ¹⁷⁷Lu-complex of Compound 57. In some embodiments, the ¹⁷⁷Lu-complex is a ¹⁷⁷Lu-complex of Compound 58. In some embodiments, the ¹⁷⁷Lu-complex is a ¹⁷⁷Lu-complex of Compound 59. In some embodiments, the ¹⁷⁷Lu-complex is a ¹⁷⁷Lu-complex of Compound 60. In some embodiments, the ¹⁷⁷Lu-complex is a ¹⁷⁷Lu-complex of Compound 61. In some embodiments, the ¹⁷⁷Lu-complex is a ¹⁷⁷Lu-complex of Compound 62. In some embodiments, the ¹⁷⁷Lu-complex is a ¹⁷⁷Lu-complex of Compound 63. In some embodiments, the ¹⁷⁷Lu-complex is a ¹⁷⁷Lu-complex of Compound 64. In some embodiments, the ¹⁷⁷Lu-complex is a ¹⁷⁷Lu-complex of Compound 65. In some embodiments, the ¹⁷⁷Lu-complex is a ¹⁷⁷Lu-complex of Compound 66. In some embodiments, the 177Lu-complex is a ¹⁷⁷Lu-complex of Compound 67. In some embodiments, the ¹⁷⁷Lu-complex is a ¹⁷⁷Lu-complex of Compound 68. In some embodiments, the ¹⁷⁷Lu-complex is a ¹⁷⁷Lu-complex of Compound 69. In some embodiments, the ¹⁷⁷Lu-complex is a ¹⁷⁷Lu-complex of Compound 70. In some embodiments, the ¹⁷⁷Lu-complex is a ¹⁷⁷Lu-complex of Compound 71. In some embodiments, the ¹⁷⁷Lu-complex is a ¹⁷⁷Lu-complex of Compound 72. In some embodiments, the ¹⁷⁷Lu-complex is a ¹⁷⁷Lu-complex of Compound 73. In some embodiments, the ¹⁷⁷Lu-complex is a ¹⁷⁷Lu-complex of Compound 74. In some embodiments, the ¹⁷⁷Lu-complex is a ¹⁷⁷Lu-complex of Compound 75. In some embodiments, the ¹⁷⁷Lu-complex is a ¹⁷⁷Lu-complex of Compound 84. In some embodiments, the 177Lu-complex is a ¹⁷⁷Lu-complex of Compound 85. In some embodiments, the ¹⁷⁷Lu-complex is a ¹⁷⁷Lu-complex of Compound 86. In some embodiments, the ¹⁷⁷Lu-complex is a ¹⁷⁷Lu-complex of Compound 87. In some embodiments, the 177Lu-complex is a ¹⁷⁷Lu-complex of Compound 88. In some embodiments, the ¹⁷⁷Lu-complex is a ¹⁷⁷Lu-complex of Compound 89. In some embodiments, the ¹⁷⁷Lu-complex is a ¹⁷⁷Lu-complex of Compound 90. In some embodiments, the ¹⁷⁷Lu-complex is a ¹⁷⁷Lu-complex of Compound 91. In some embodiments, the ¹⁷⁷Lu-complex is a ¹⁷⁷Lu-complex of Compound 92. In some embodiments, the ¹⁷⁷Lu-complex is a ¹⁷⁷Lu-complex of Compound 93. In some embodiments, the ¹⁷⁷Lu-complex is a ¹⁷⁷Lu-complex of Compound 94. In some embodiments, the ¹⁷⁷Lu-complex is a ¹⁷⁷Lu-complex of Compound 95. In some embodiments, the ¹⁷⁷Lu-complex is a ¹⁷⁷Lu-complex of Compound 96. In some embodiments, the ¹⁷⁷Lu-complex is a ¹⁷⁷Lu-complex of Compound 97. In some embodiments, the ¹⁷⁷Lu-complex is a ¹⁷⁷Lu-complex of Compound 98. In some embodiments, the ¹⁷⁷Lu-complex is a ¹⁷⁷Lu-complex of Compound 99. In some embodiments, the ¹⁷⁷Lu-complex is a ¹⁷⁷Lu-complex of Compound 100. In some embodiments, the ¹⁷⁷Lu-complex is a ¹⁷⁷Lu-complex of Compound 101. In some embodiments, the ¹⁷⁷Lu-complex is a ¹⁷⁷Lu-complex of Compound 102. In some embodiments, the ¹⁷⁷Lu-complex is a ¹⁷⁷Lu-complex of Compound 103. In some embodiments, the ¹⁷⁷Lu-complex is a ¹⁷⁷Lu-complex of Compound 104. In some embodiments, the 77Lu-complex is a ¹⁷⁷Lu-complex of Compound 105. In some embodiments, the ¹⁷⁷Lu-complex is a ¹⁷⁷Lu-complex of Compound 106. In some embodiments, the ¹⁷⁷Lu-complex is a ¹⁷⁷Lu-complex of Compound 107. In some embodiments, the ¹⁷⁷Lu-complex is a ¹⁷⁷Lu-complex of Compound 108. In some embodiments, the ¹⁷⁷Lu-complex is a ¹⁷⁷Lu-complex of Compound 109. In some embodiments, the ¹⁷⁷Lu-complex is a ¹⁷⁷Lu-complex of Compound 110. In some embodiments, the ¹⁷⁷Lu-complex is a ¹⁷⁷Lu-complex of Compound 111. In some embodiments, the ¹⁷⁷Lu-complex is a ¹⁷⁷Lu-complex of Compound 112. In some embodiments, the ¹⁷⁷Lu-complex is a ¹⁷⁷Lu-complex of Compound 113. In some embodiments, the ¹⁷⁷Lu-complex is a ¹⁷⁷Lu-complex of Compound 114. In some embodiments, the ¹⁷⁷Lu-complex is a ¹⁷⁷Lu-complex of Compound 115. In some embodiments, the ¹⁷⁷Lu-complex is a ¹⁷⁷Lu-complex of Compound 116. In some embodiments, the ¹⁷⁷Lu-complex is a 177Lu-complex of Compound 117. In some embodiments, the ¹⁷⁷Lu-complex is a ¹⁷⁷Lu-complex of Compound 118. In some embodiments, the ¹⁷⁷Lu-complex is a ¹⁷⁷Lu-complex of Compound 119. In some embodiments, the ¹⁷⁷Lu-complex is a ¹⁷⁷Lu-complex of Compound 120. In some embodiments, the ¹⁷⁷Lu-complex is a ¹⁷⁷Lu-complex of Compound 121. In some embodiments, the ¹⁷⁷Lu-complex is a ¹⁷⁷Lu-complex of Compound 122. In some embodiments, the ¹⁷⁷Lu-complex is a ¹⁷⁷Lu-complex of Compound 123. In some embodiments, the ¹⁷⁷Lu-complex is a ¹⁷⁷Lu-complex of Compound 124. In some embodiments, the ¹⁷⁷Lu-complex is a ¹⁷⁷Lu-complex of Compound 137. In some embodiments, the ¹⁷⁷Lu-complex is a ¹⁷⁷Lu-complex of Compound 138. In some embodiments, the ¹⁷⁷Lu-complex is a ¹⁷⁷Lu-complex of Compound 139. In some embodiments, the ¹⁷⁷Lu-complex is a 177Lu-complex of Compound 140. In some embodiments, the ¹⁷⁷Lu-complex is a ¹⁷⁷Lu-complex of Compound 141. In some embodiments, the ¹⁷⁷Lu-complex is a ¹⁷⁷Lu-complex of Compound 142. In some embodiments, the ¹⁷⁷Lu-complex is a ¹⁷⁷Lu-complex of Compound 143. In some embodiments, the ¹⁷⁷Lu-complex is a ¹⁷⁷Lu-complex of Compound 144. In some embodiments, the ¹⁷⁷Lu-complex is a ¹⁷⁷Lu-complex of Compound 145. In some embodiments, the ¹⁷⁷Lu-complex is a ¹⁷⁷Lu-complex of Compound 146. In some embodiments, the ¹⁷⁷Lu-complex is a ¹⁷⁷Lu-complex of Compound 147. In some embodiments, the ¹⁷⁷Lu-complex is a ¹⁷⁷Lu-complex of Compound 148. In some embodiments, the ¹⁷⁷Lu-complex is a ¹⁷⁷Lu-complex of Compound 149. In some embodiments, the ¹⁷⁷Lu-complex is a ¹⁷⁷Lu-complex of Compound 150. In some embodiments, the ¹⁷⁷Lu-complex is a ¹⁷⁷Lu-complex of Compound 151. In some embodiments, the ¹⁷⁷Lu-complex is a ¹⁷⁷Lu-complex of Compound 152. In some embodiments, the ¹⁷⁷Lu-complex is a ¹⁷⁷Lu-complex of Compound 153. In some embodiments, the ¹⁷⁷Lu-complex is a ¹⁷⁷Lu-complex of Compound 154. In some embodiments, the ¹⁷⁷Lu-complex is a ¹⁷⁷Lu-complex of Compound 155. In some embodiments, the ¹⁷⁷Lu-complex is a ¹⁷⁷Lu-complex of Compound 156. In some embodiments, the ¹⁷⁷Lu-complex is a ¹⁷⁷Lu-complex of Compound 157. In some embodiments, the ¹⁷⁷Lu-complex is a ¹⁷⁷Lu-complex of Compound 158. In some embodiments, the ¹⁷⁷Lu-complex is a ¹⁷⁷Lu-complex of Compound 159. In some embodiments, the ¹⁷⁷Lu-complex is a ¹⁷⁷Lu-complex of Compound 160.

Dose: 5.0 mCi (±20%) for ⁶⁸Ga complex. 20 mCi (±20%) to 27 mCi (±20%) for ¹⁷⁷Lu-complex; Total carrier mass of the compound of Formula (I) or Formula (A): not more than (NMT) 90 μg/dose.

Mode of administration: Intravenous

Duration of Participation: A 28-day screening window will be utilized where the subject will undergo study assessments to deem the subject eligible for the study. Once confirmed, subjects will receive the study drug, ⁶⁸Ga or ¹⁷⁷Lu-complex of a compound of Formula (I) or Formula (A), and PET/CT or SPECT/CT imaging on days described in protocol. The subjects will have a safety follow-up up to 7 days after last imaging assessment.

Study Duration: The start of the study will be the date on which the first subject provides informed consent. The end of the study will be when the database is locked.

The examples and embodiments described herein are for illustrative purposes only and various modifications or changes suggested to persons skilled in the art are to be included within the purview of this application and scope of the appended claims.

1. A compound of the following chemical formula, or a pharmaceutically acceptable salt thereof: 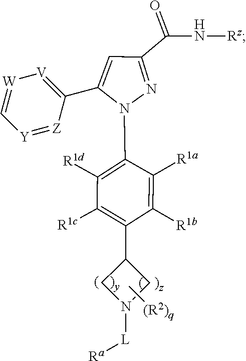
wherein:
R^{1a}, R^{1b}, R^{1c}, and R^{1d} are each independently selected from the group consisting of —F, —Cl, —Br, —I, —OH, substituted or unsubstituted alkyl, substituted or unsubstituted heteroalkyl, —CF₃, —CN, —CO₂H, —CO₂CH₃, —C(═O)NR³R⁴, —CH(═N)OH, —NR⁵R⁶, —NO₂, —NR⁷C(═O)R⁸, —NR⁹C(═O)OR⁰, and —OC(═O)NR¹¹R¹²;
R³, R⁴, R⁵, R⁶, R⁷, R⁸, R⁹, R¹⁰, R¹¹ and R¹² are each independently —H or —CH₃;
each R² is independently selected from the group consisting of —F, substituted or unsubstituted C₁-C₆ alkyl, —CF₃, or —CN, or two R² variables on the same or adjacent carbon atoms come together to form an unsubstituted C₃-C₆ cycloalkyl ring;
y is 1 or 2;
z is 1 or 2;
q is 0, 1, 2, 3, 4, 5, 6, 7, or 8;
W is —C(R^{r})— or —N— and Y is —C(R^{s})— or —N—; wherein R^{r} and R^{s} are each independently selected from the group consisting of: hydrogen, —F, —Cl, —Br, —CF₃, —C₁-C₆ alkyl, and —O—C₁-C₆ alkyl;
V is —C(R^{x})— or —N— and Z is —C(R^{y})— or —N—, wherein R^{x} and R^{y} are each independently selected from the group consisting of: —F, —Cl, —Br, —CF₃, —C₁-C₆ alkyl, or —O—C₁-C₆ alkyl;
R^{z} is 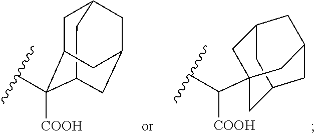 [SMILES: *C1(C(=O)O)C2CC3CC(C2)CC1C3 ; *C(C(=O)O)C12CC3CC(CC(C3)C1)C2]
L is a linker; and
R^{a} is a chelating moiety or a radionuclide complex thereof.

2. The compound of claim 1- or a pharmaceutically acceptable salt thereof, wherein:
W is —N— and Y is —CH— or wherein W and Y are —CH—; and
V is —C(R^{x})—; Z is —C(R^{y})—; R^{x} is —F, —Cl, or —OCH₃; and R^{y} is —OCH₃.

3. The compound of claim 1, or a pharmaceutically acceptable salt thereof, wherein R^{1a}, R^{1b}, R^{1c}, and R^{1d} are each independently selected from the group consisting of —F, —Cl, —Br, —I, —OH and —C₁-C₆ alkyl.

4. The compound of claim 1, or a pharmaceutically acceptable salt thereof, wherein R^{1a} is —CH(CH₃)₂.

5. The compound of claim 1, or a pharmaceutically acceptable salt thereof, wherein q is 0.

6. The compound of claim 1, or a pharmaceutically acceptable salt thereof, wherein: 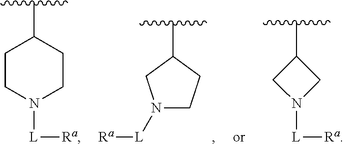
is

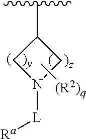

7. The compound of claim 1, or a pharmaceutically acceptable salt thereof, wherein the compound has the structure of Formula (IIa), Formula (IIIa), Formula (IVa), Formula (XIIa), or Formula (IIc): 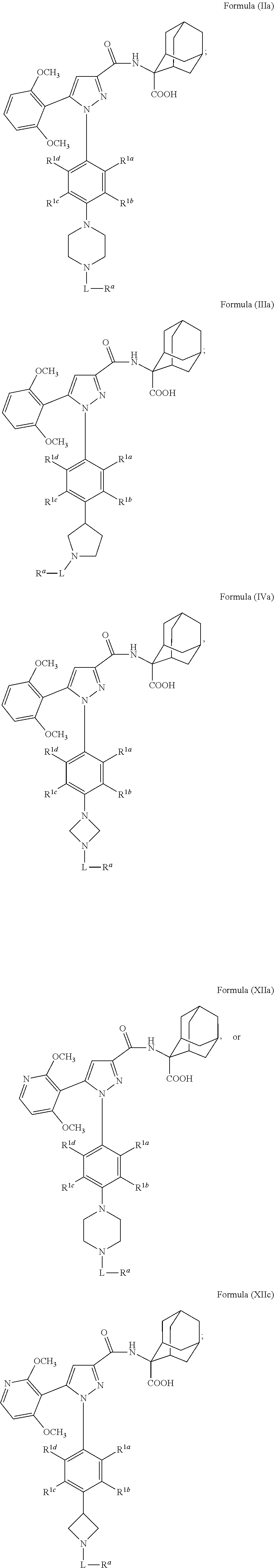
wherein R^{1a}, R^{1b}, R^{1c}, and R^{1d} are each independently selected from the group consisting of hydrogen, —F, —Cl, —Br, —I, —OH, —CF₃, —CN, —CO₂H, —C(═O)OCH₃, —C(═O)NH₂, —CH(═N)OH, —NH₂, —NHCH₃, —N(CH₃)₂, —NO₂, —NHC(═O)CH₃, —NHCO₂H, —NHC(═O)OCH₃, —OC(═O)NH₂, and substituted or unsubstituted —C₁-C₆ alkyl.

8. The compound of claim 1, or a pharmaceutically acceptable salt thereof, wherein the compound has the following structure: 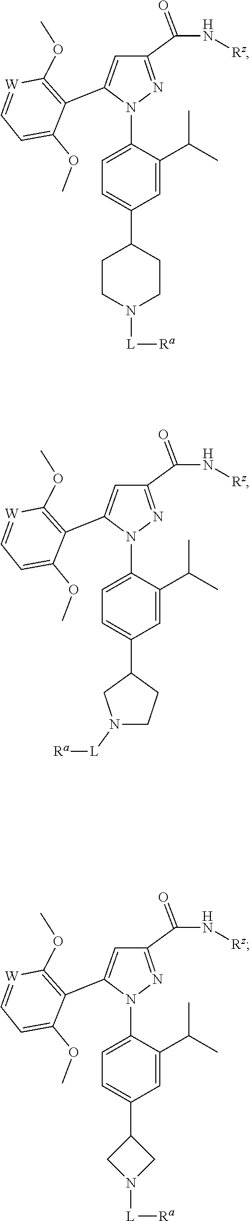
wherein
W is —CH— or —N—, and R^{z} is

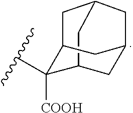 [SMILES: *C1(C(=O)O)C2CC3CC(C2)CC1C3]

9. The compound of claim 1, or a pharmaceutically acceptable salt thereof, wherein R^{a} is a chelating moiety independently selected from the group consisting of:
1,4,7,10-tetraazacyclododecane-1,4,7,10-tetraacetic acid (DOTA);
2,2′,2″-(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DO3A);
1,4,7,10-tetraazacyclododecane-1,7-diacetic acid (DO2A);
α,α′,α″,a′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
6,6′-(((pyridine-2,6-diylbis(methylene))bis((carboxymethyl)azanediyl))bis(methylene))dipicolinic acid (H₄pypa);
H₄pypa-benzyl;
6,6′,6″,6′″-(((pyridine-2,6-diylbis(methylene))bis(azanetriyl))tetrakis(methylene))-tetrapicolinic acid (H₄py4pa);
H₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2′″-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; and
3,6,9,12-tetrakis(carboxymethyl)-3,6,9,12-tetraazatetradecanedioic acid (TTHA);
or a radionuclide complex thereof.

10. The compound of claim 1, or a pharmaceutically acceptable salt thereof, wherein R^{a} is 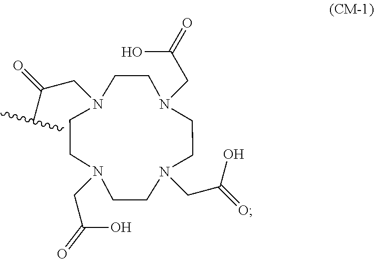 [SMILES: *C(=O)CN1CCN(CC(=O)O)CCN(CC(=O)O)CCN(CC(=O)O)CC1]
or a radionuclide complex thereof.

11. The compound of claim 1, wherein L is: -L², -L⁴-, -L²-L⁴-, -L²-L⁶-, -L³-L⁶-, -L²-L³-L⁴-, -L²-L³-L⁶-, -L²-L⁵-L⁶-, -L²-L³-L⁵-L⁶- or -L²-L³-L⁵-L⁶-;
L² is absent, —C₁-C₂₀ alkylene, —C₁-C₂₀ alkylene-NR¹³—, —C₁-C₂₀ alkylene-NR¹³—C(═O)—, —NR¹³—C₁-C₂₀alkylene-, —NR¹³—C₁-C₂₀alkylene-NR¹⁶—, —C₁-C₂₀ alkylene-O—, —O—C₁-C₂₀ alkylene-, —O—C₁-C₂₀ alkylene-NR¹⁶—, —NR¹³C(═O)—C₁-C₂₀ alkylene-NR¹⁶—, —C₁-C₂₀ alkylene-C(═O)—, —C(═O)—C₁-C₂₀ alkylene-, —C(═O)—C₁-C₂₀ alkylene-NR¹³—, —C(═O)—NR¹³—C₁-C₂₀ alkylene-NR¹⁶—, —C(═NH)NH—C₁-C₂₀ alkylene-NR¹³—, —C(═NH)NH—C₁—C₂₀ alkylene-, or —C(═O)—NH—O—(CH₂)ᵥ—; wherein each C₁-C₂₀ alkylene is optionally substituted with 1-3 substituents selected from the group consisting of —OH, —NH₂, and —COOH;
each R¹³ and R¹⁶ are independently selected from H or —C₁-C₄ alkyl;
each v is independently 1, 2, 3, 4, 5 or 6;
L³ is absent, —C₁-C₂₀ alkylene-, —C₁-C₂₀ alkylene-NR¹⁴—, —C(═O)—C₁-C₂₀ alkylene-, —(CH₂CH₂O)—CH₂CH₂—, —C(═O)—(CH₂CH₂O)_{w}—CH₂CH₂—, —(CH₂CH₂NR⁴),—CH₂CH₂—, —C(═O)NR¹⁴—(CH₂CH₂NR¹⁴),—CH₂CH₂—, —C(═O)—NH—O—(CH₂)_{w}—, or —C(═O)—(CH₂)_{w}—O—NH—(CH₂)w-C(═O)—; wherein each C₁-C₂₀ alkylene is optionally substituted with 1-3 substituents selected from the group consisting of —OH and —NH₂;
each R⁴ is independently H or —C₁-C₆ alkyl;
each w is independently 1, 2, 3, 4, 5 or 6;
L⁴ is absent, a natural or unnatural amino acid, or a peptide that is formed from two or more independently selected natural and unnatural amino acids, wherein when two or more amino acids are present then the N atom of the amide linking the amino acids is optionally substituted with —C₁-C₆ alkyl;
L⁵ is absent or -L⁷-L⁸-L⁹-;
L⁷ is absent, —O—, —S—, —NH— or —NCH₃—;
L⁸ is substituted or unsubstituted cycloalkylene, substituted or unsubstituted cycloalkenylene, or substituted or unsubstituted heterocycloalkylene;
L⁹ is absent, —(CH₂)ₓ—, —C(═O)(CH₂)ₓ—, or —NR¹⁵(CH₂)ₓ—;
x is 1, 2, 3, 4, 5 or 6;
each R¹⁵ is independently selected from H or —C₁-C₆ alkyl; and
L⁶ is absent, —NH—, or —N(CH₃)—;
wherein if L⁶ is absent, L⁴ cannot be absent.

12. The compound of claim 11, wherein L² is absent, —C₁-C₆ alkylene, —C₁-C₆alkylene-NR¹³—, —C₁-C₂ alkylene-NR³—C(═O)—, —NR¹³—C₁-C₂ alkylene-, —NR¹³—C₁-C₂₀ alkylene-NR¹⁶—, —C₁-C₆ alkylene-C(═O)—, —C(═O)—C₁-C₆ alkylene-, —C(═O)—C₁-C₆ alkylene-NR¹³—, —C(═O)—NR¹³—C₁-C₂₀ alkylene-NR¹⁶—, or —C(═NH)NH—C₁-C₂₀ alkylene-NR¹³—, wherein each C₁-C₆ alkylene is optionally substituted with 1 —OH, —NH₂, and —COOH.

13. The compound of claim 11, wherein L² is —C₁-C₂₀ alkylene, —C₁-C₆ alkylene-NR¹³—C(═O)— or —C₁-C₆ alkylene-NR¹³—, wherein R¹³ is H or —CH₃.

14. The compound of claim 11, wherein L³ is absent, —C₁-C₆ alkylene-, —C₁-C₆ alkylene-NR¹⁴—, or —C(═O)—C₁-C₆ alkylene-, wherein the C₁-C₆ alkylene is optionally substituted with 1 —OH or —NH₂.

15. The compound of claim 11, or a pharmaceutically acceptable salt thereof, wherein L⁴ and L⁵ are absent.

16. The compound of claim 11, or a pharmaceutically acceptable salt thereof, wherein L⁵ is -L⁷-L⁸-L⁹-;
L⁷ is absent, —NH—, or —NCH₃—;
L⁸ is substituted or unsubstituted cycloalkenylene or substituted or unsubstituted heterocycloalkylene;
L⁹ is absent, —C(═O)(CH₂)ₓ—, or —NR¹⁵(CH₂)ₓ—;
x is 1, 2, 3, 4, 5 or 6; and
each R¹⁵ is independently selected from H or —CH₃.

17. The compound of claim 11, or a pharmaceutically acceptable salt thereof wherein L⁶ is —NH— or —N(CH₃)—.

18. The compound of claim 1, wherein L of R^{a}-L- is:

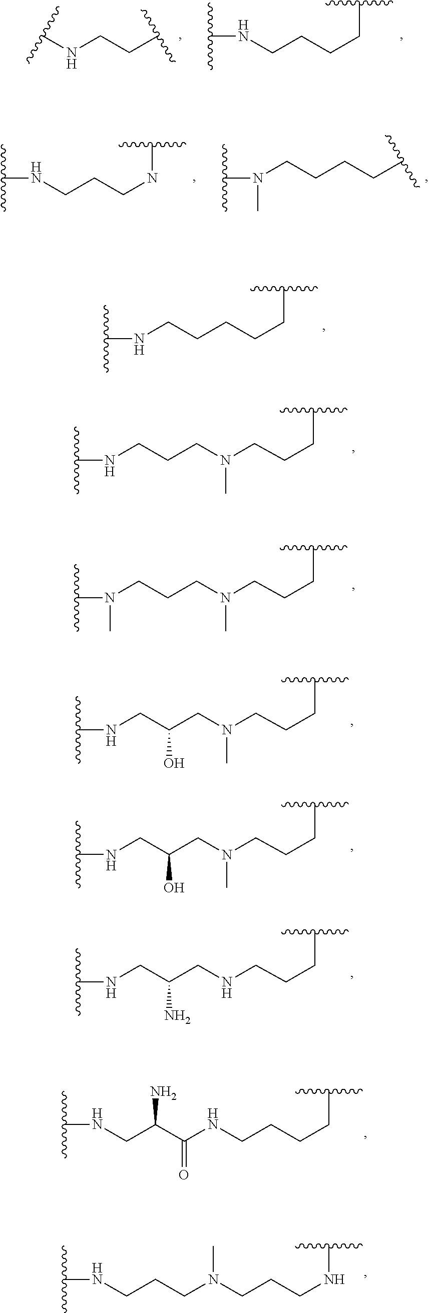

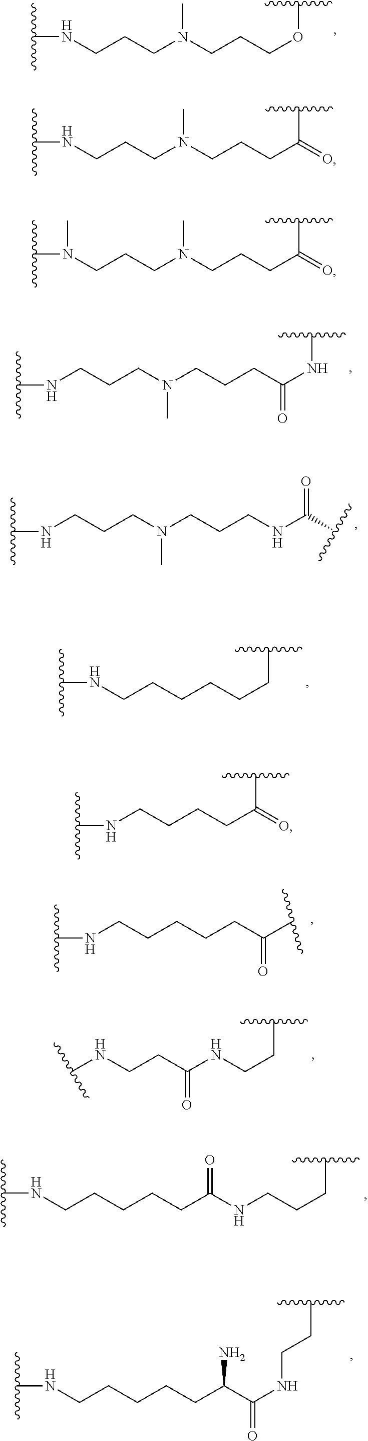

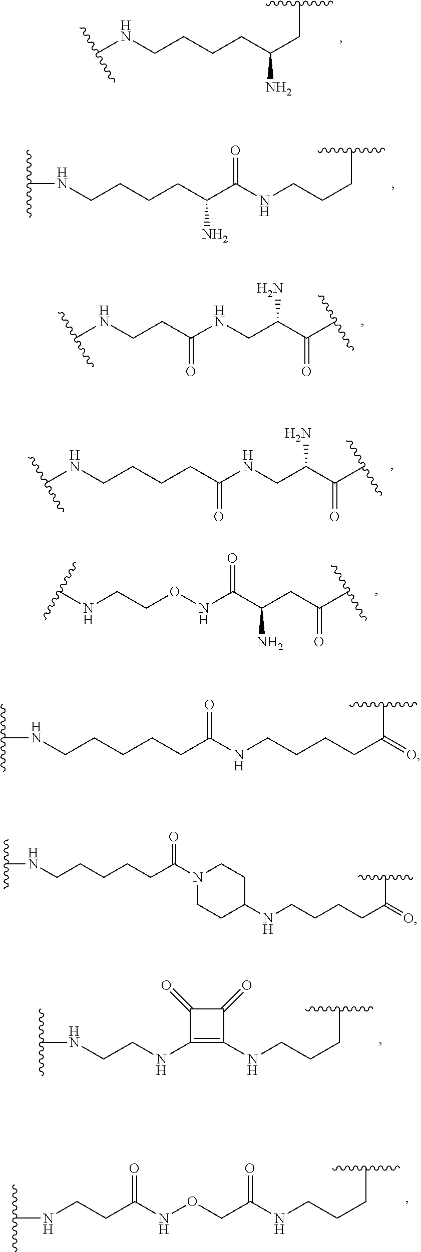

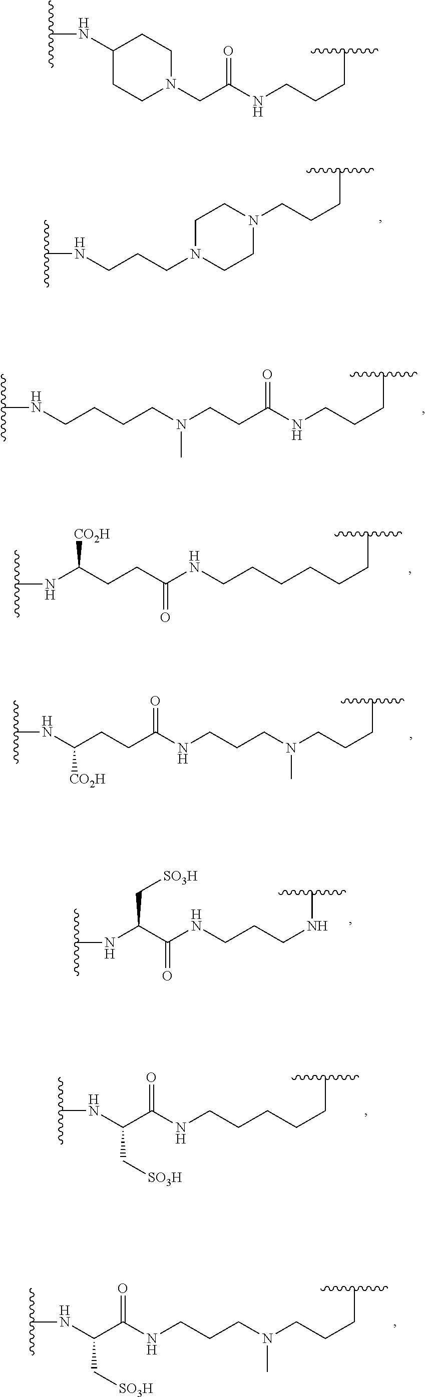

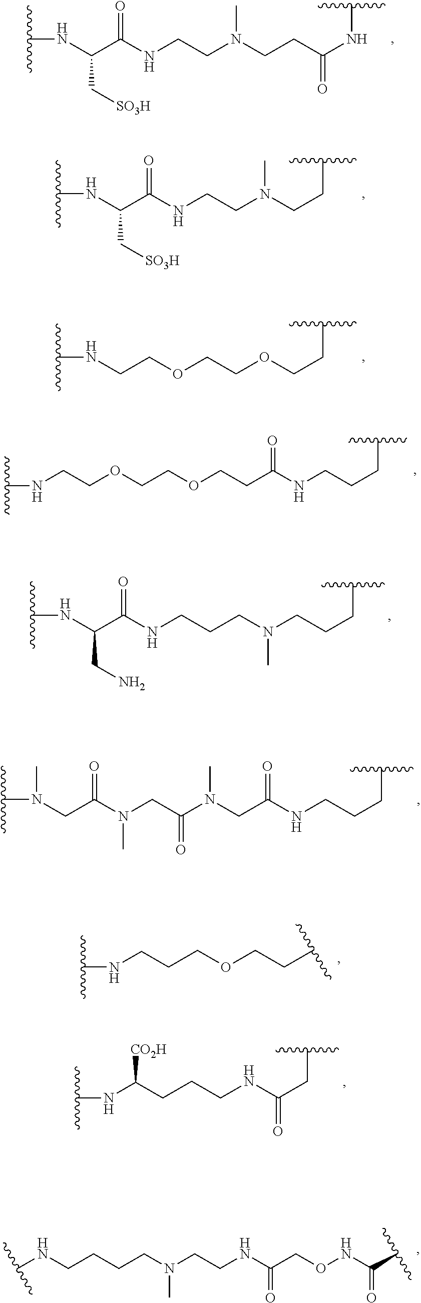

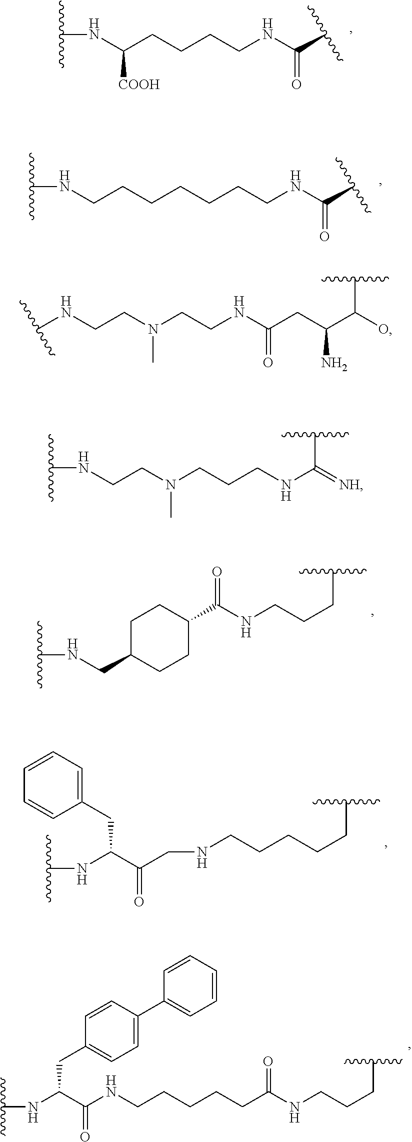

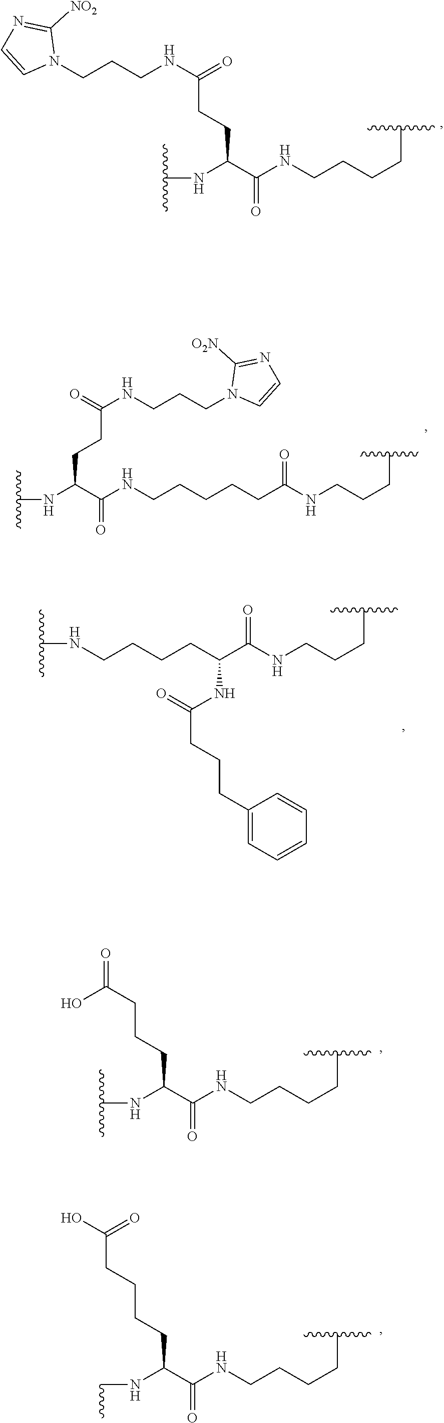

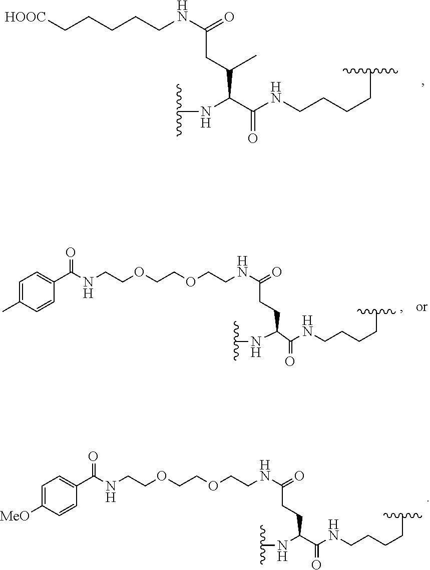 [SMILES: *CCCCNC(=O)[C@@H](N*)C(C)CC(=O)NCCCCCC(=O)O ; *CCCCNC(=O)[C@H](CCC(=O)NCCOCCOCCNC(=O)c1ccc(C)cc1)N* ; *CCCCNC(=O)[C@H](CCC(=O)NCCOCCOCCNC(=O)c1ccc(OC)cc1)N*]

19. The compound of claim 1, wherein L of R^{a}-L- is:

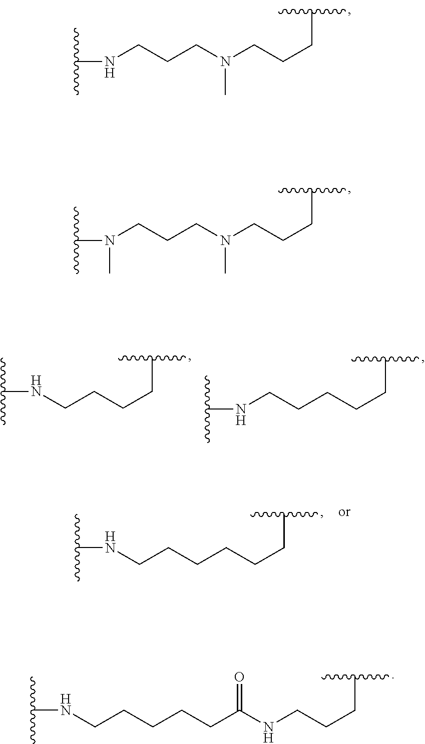 [SMILES: *CCCN(C)CCCN* ; *CCCN(C)CCCN(*)C ; *CCCCN* ; *CCCCCN* ; *CCCCCCN* ; *CCCNC(=O)CCCCCN*]

20. The compound of claim 1, wherein R^{a}-L- is

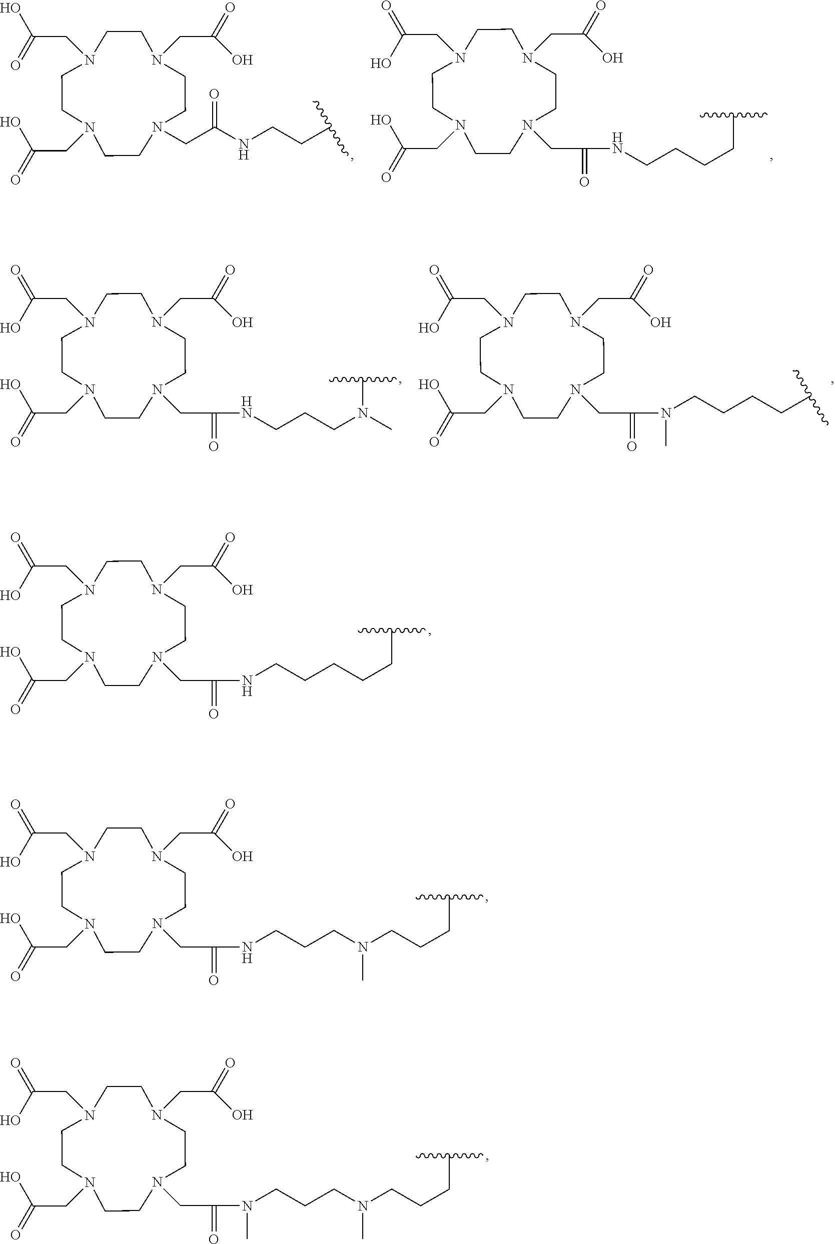

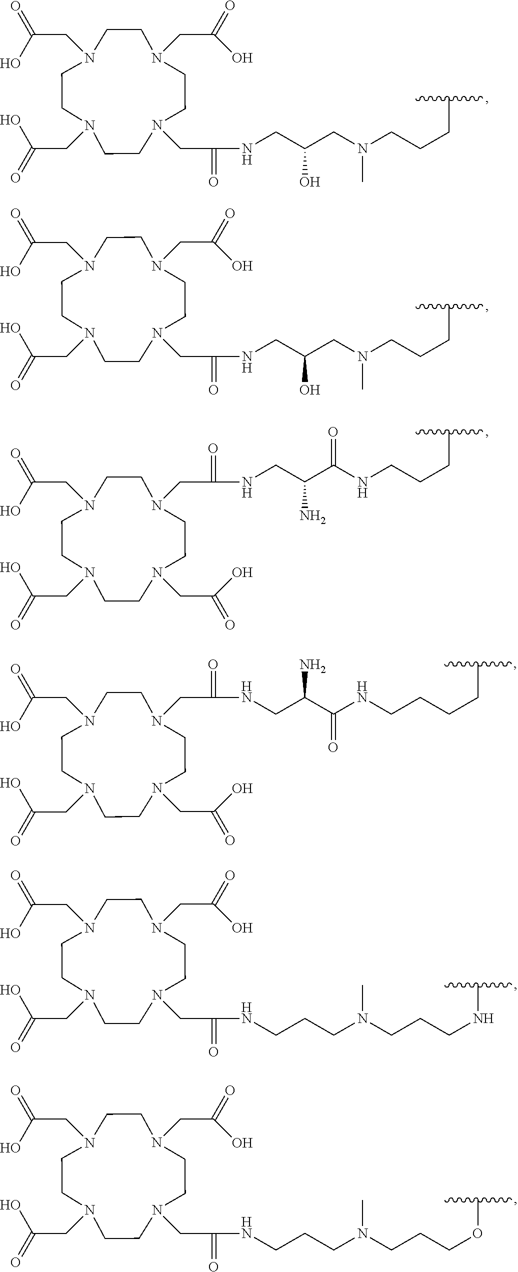

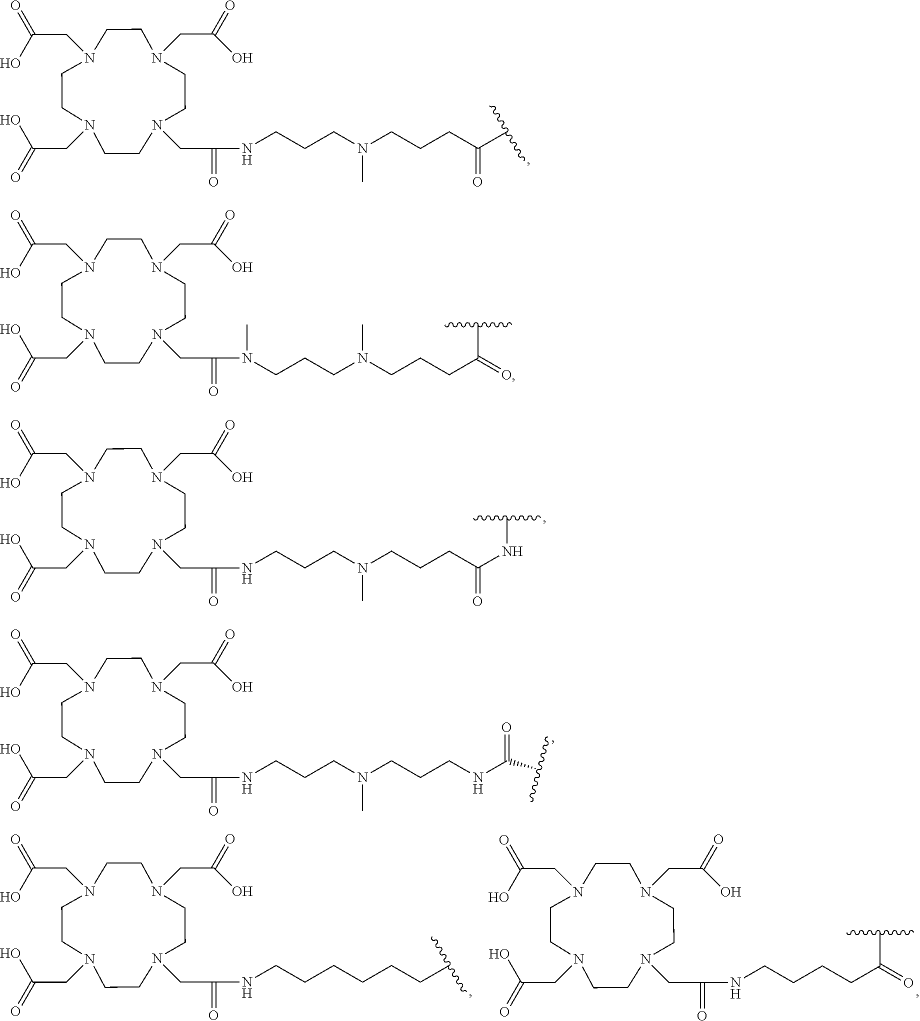

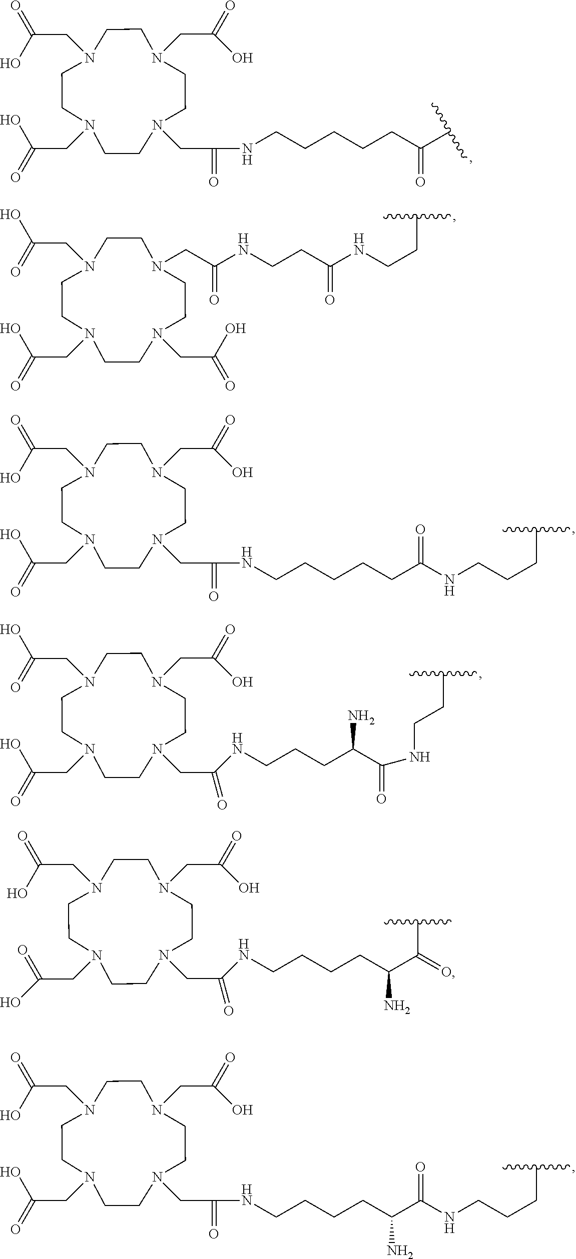

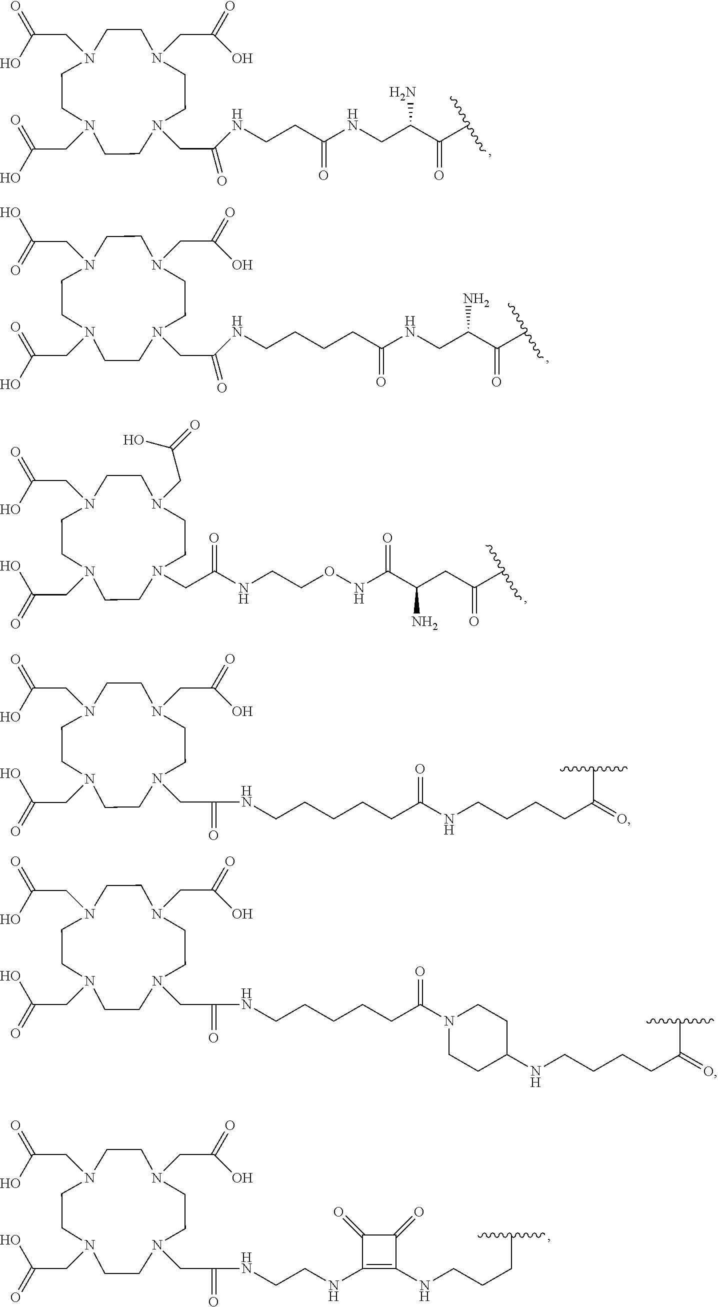

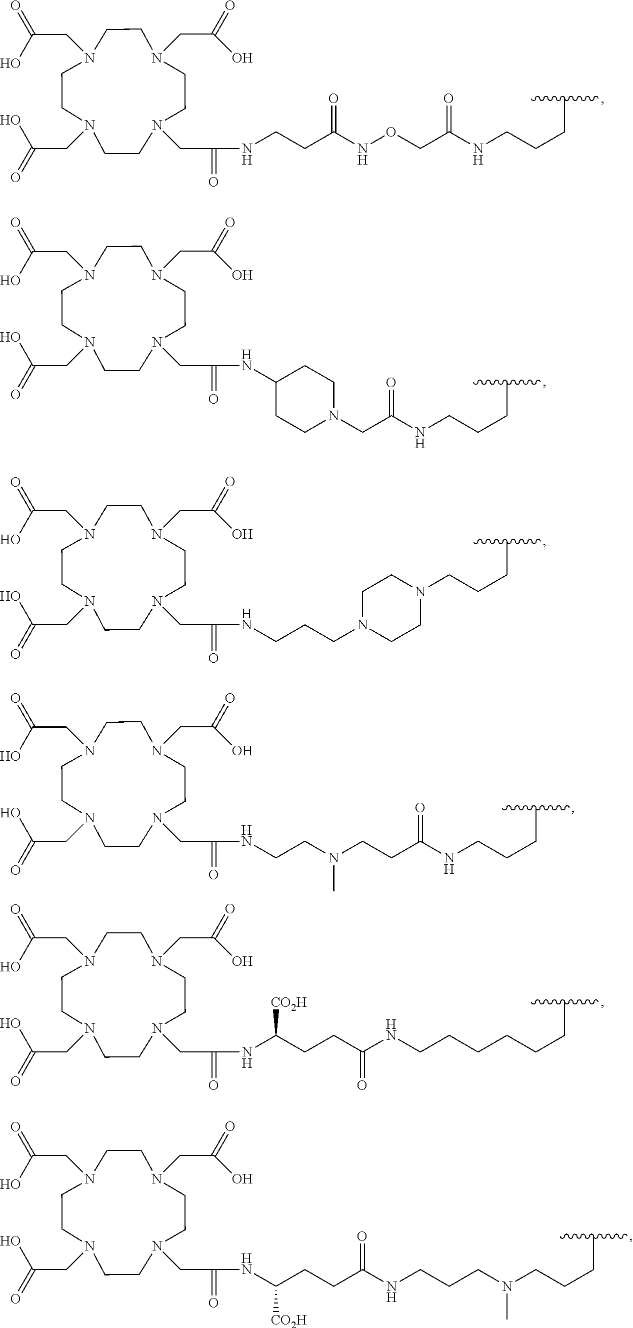

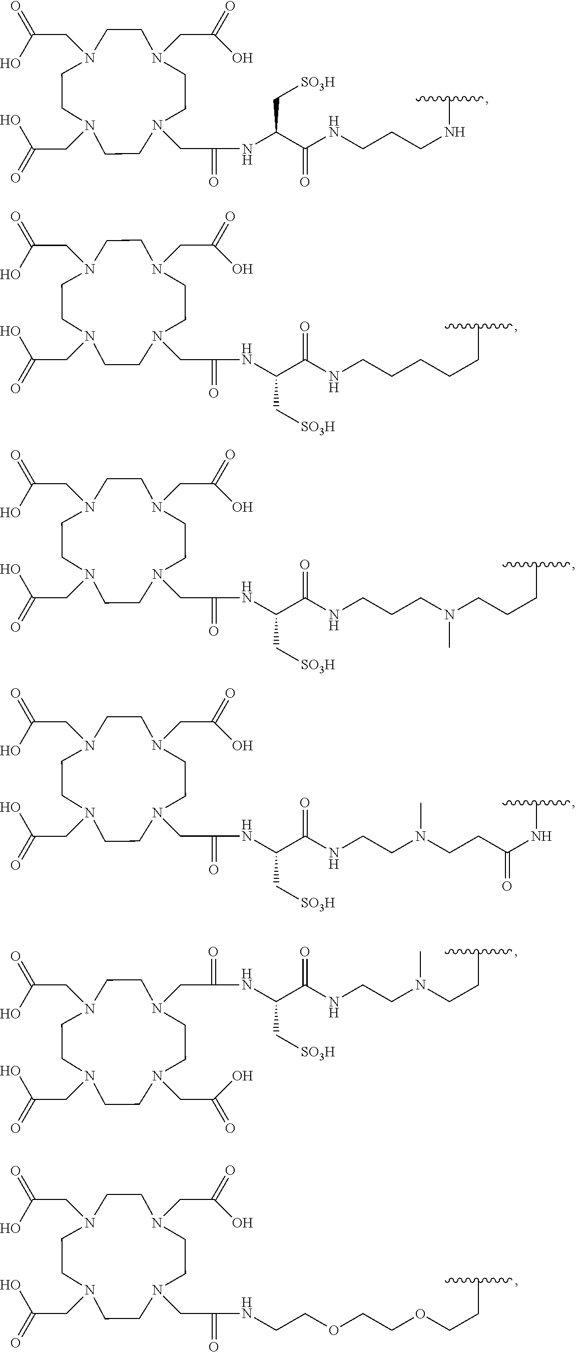

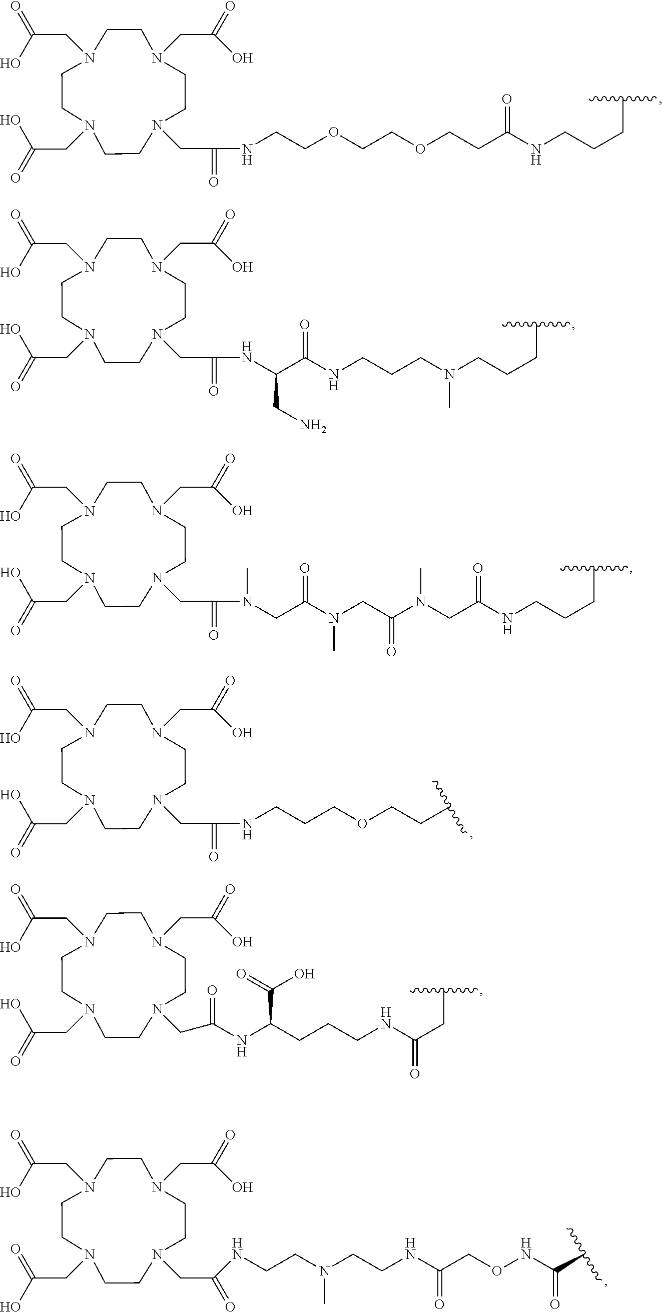

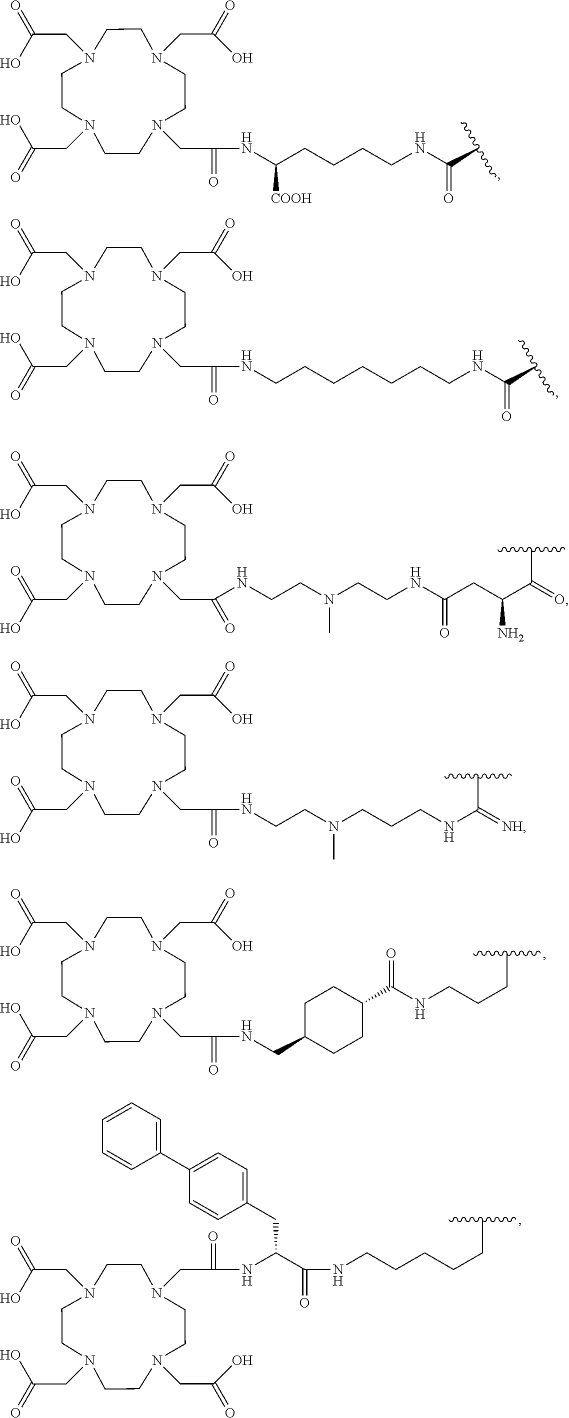

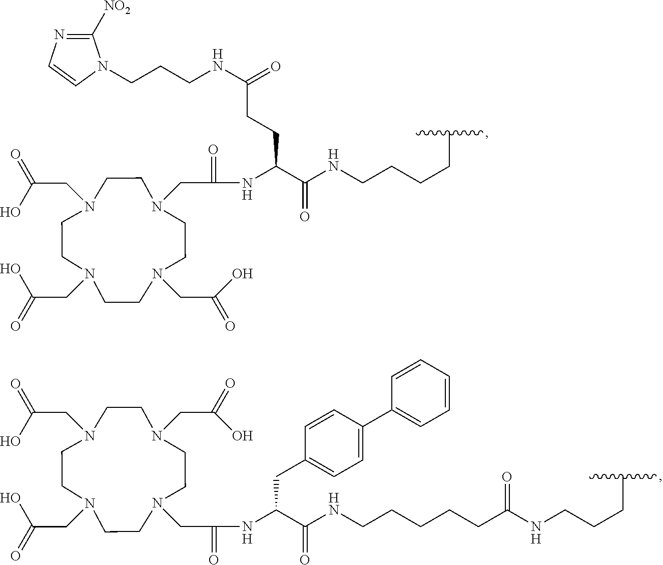 [SMILES: *CCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)CCCCCNC(=O)[C@@H](Cc1ccc(-c2ccccc2)cc1)NC(=O)CN1CCN(CC(=O)O)CCN(CC(=O)O)CCN(CC(=O)O)CC1]

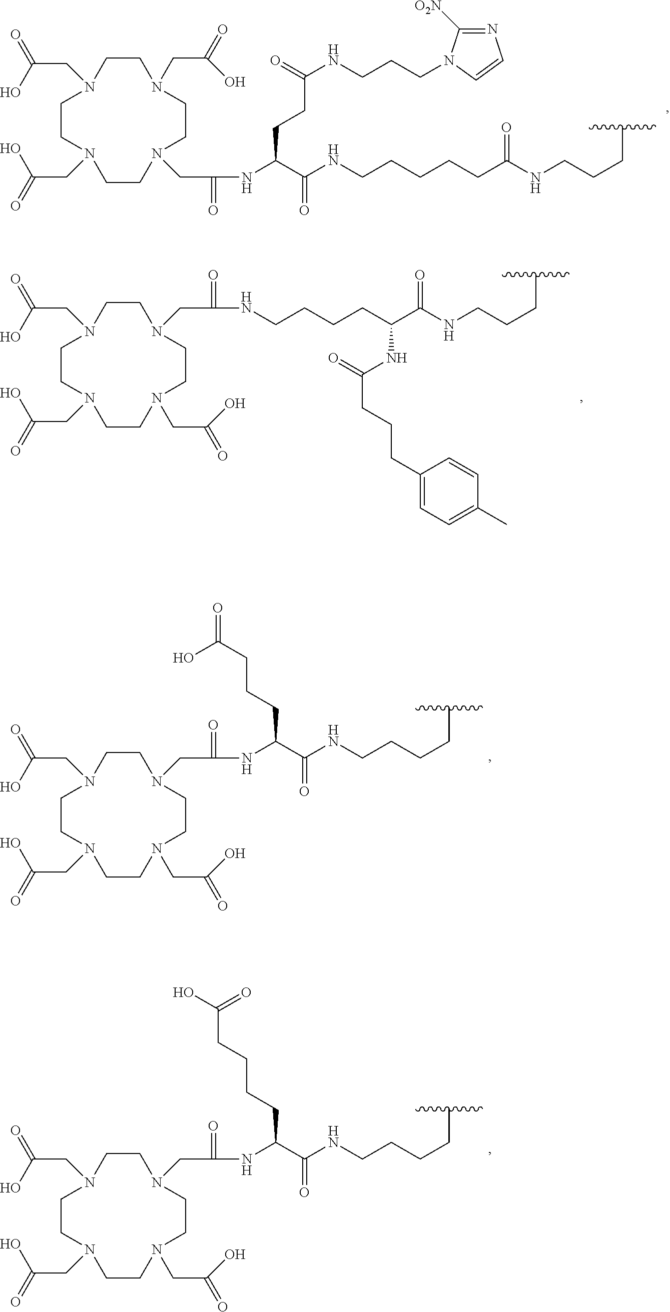 [SMILES: *CCCNC(=O)CCCCCNC(=O)[C@H](CCC(=O)NCCCn1ccnc1[N+](=O)[O-])NC(=O)CN1CCN(CC(=O)O)CCN(CC(=O)O)CCN(CC(=O)O)CC1 ; *CCCNC(=O)[C@@H](CCCCNC(=O)CN1CCN(CC(=O)O)CCN(CC(=O)O)CCN(CC(=O)O)CC1)NC(=O)CCCc1ccc(C)cc1 ; *CCCCNC(=O)[C@H](CCCC(=O)O)NC(=O)CN1CCN(CC(=O)O)CCN(CC(=O)O)CCN(CC(=O)O)CC1 ; *CCCCNC(=O)[C@H](CCCCC(=O)O)NC(=O)CN1CCN(CC(=O)O)CCN(CC(=O)O)CCN(CC(=O)O)CC1]

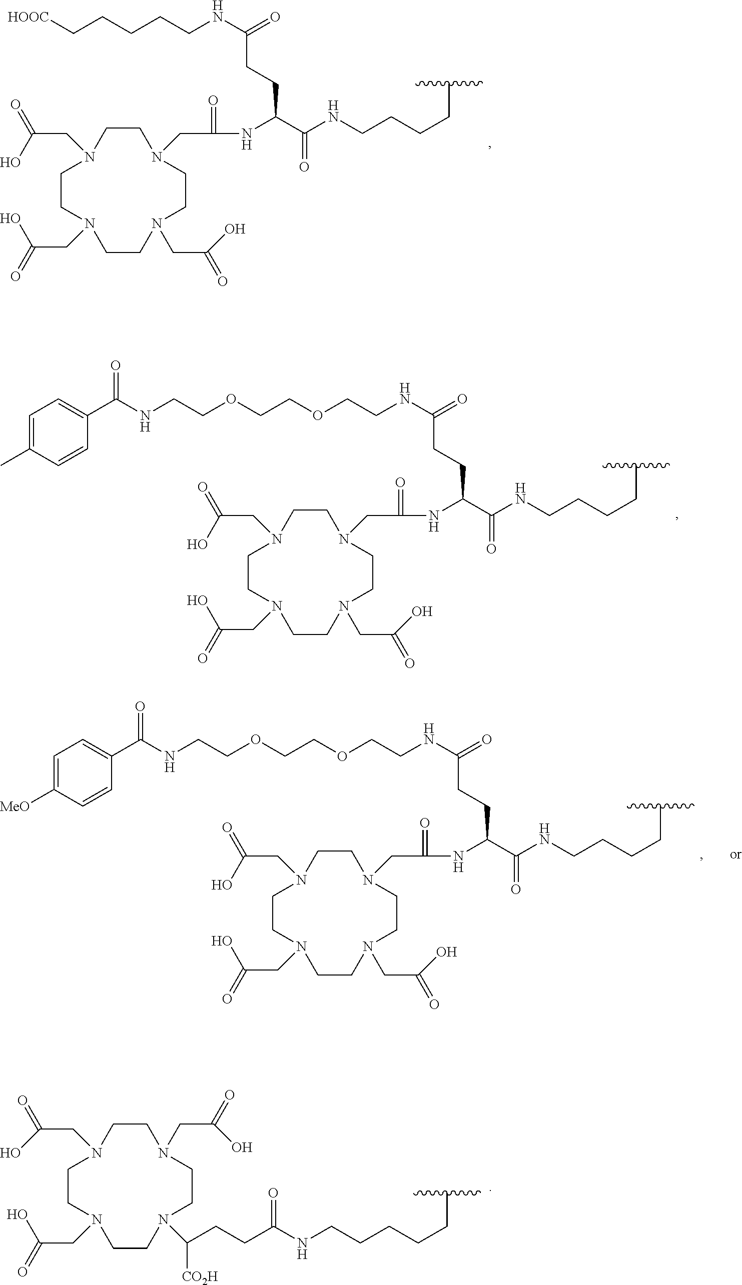 [SMILES: *CCCCNC(=O)[C@H](CCC(=O)NCCCCCC(=O)O)NC(=O)CN1CCN(CC(=O)O)CCN(CC(=O)O)CCN(CC(=O)O)CC1 ; *CCCCNC(=O)[C@H](CCC(=O)NCCOCCOCCNC(=O)c1ccc(C)cc1)NC(=O)CN1CCN(CC(=O)O)CCN(CC(=O)O)CCN(CC(=O)O)CC1 ; *CCCCNC(=O)[C@H](CCC(=O)NCCOCCOCCNC(=O)c1ccc(OC)cc1)NC(=O)CN1CCN(CC(=O)O)CCN(CC(=O)O)CCN(CC(=O)O)CC1 ; *CCCCCNC(=O)CCC(C(=O)O)N1CCN(CC(=O)O)CCN(CC(=O)O)CCN(CC(=O)O)CC1]

21. The compound of claim 1, or a pharmaceutically acceptable salt thereof, wherein the compound has one of the following structures, or a pharmaceutically acceptable salt thereof: 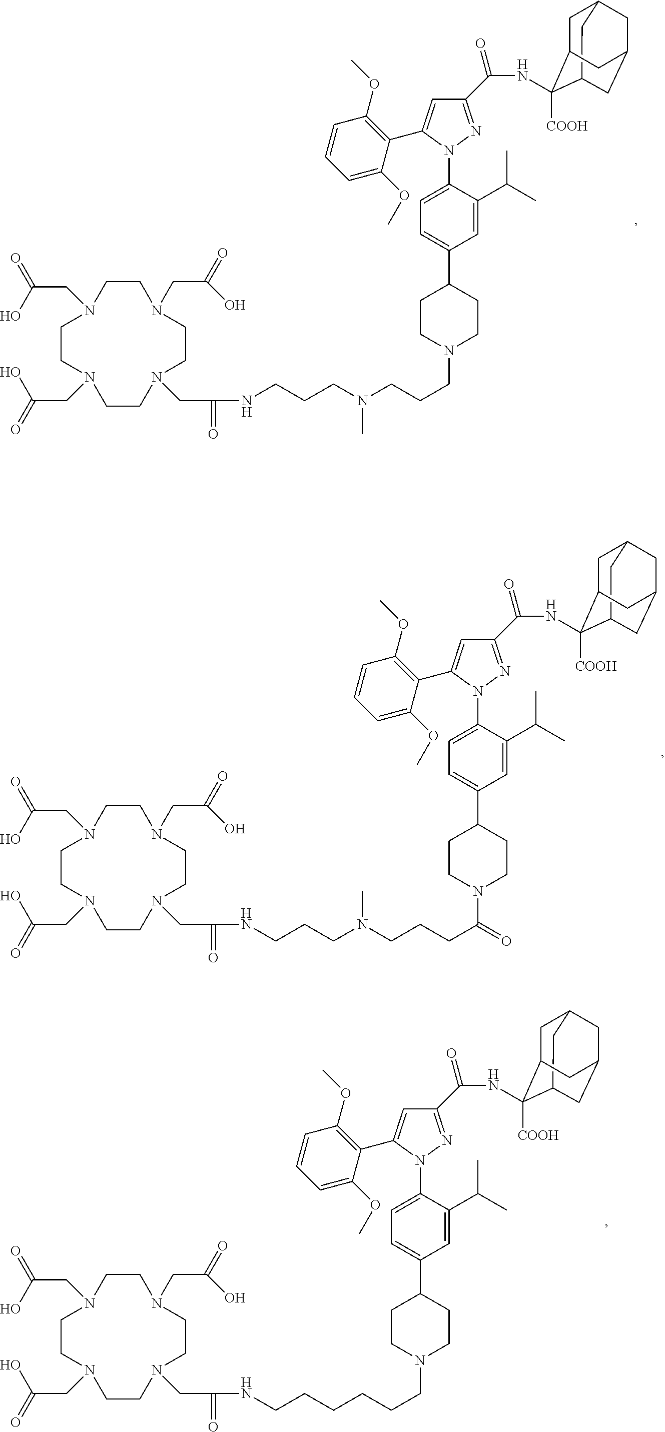 [SMILES: COc1cccc(OC)c1-c1cc(C(=O)NC2(C(=O)O)C3CC4CC(C3)CC2C4)nn1-c1ccc(C2CCN(CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCN(C)CCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCCCNC(=O)CN3CCN(CC(=O)O)CCN(CC(=O)O)CCN(CC(=O)O)CC3)CC2)cc1C(C)C] 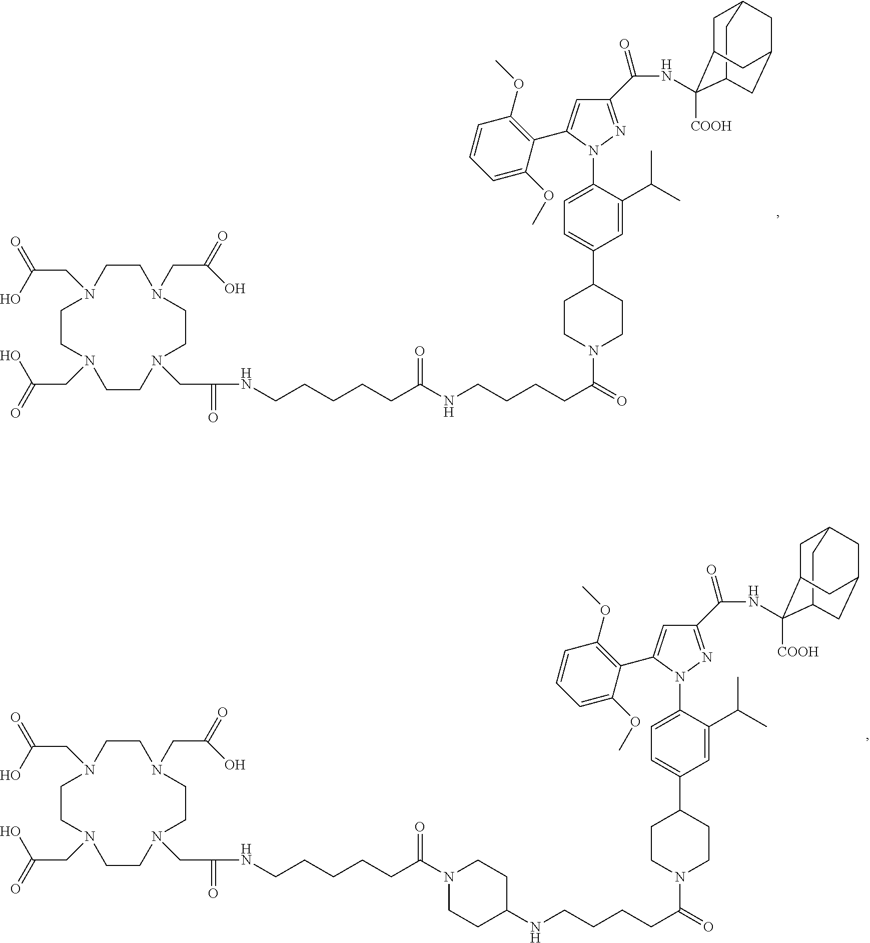 [SMILES: COc1cccc(OC)c1-c1cc(C(=O)NC2(C(=O)O)C3CC4CC(C3)CC2C4)nn1-c1ccc(C2CCN(C(=O)CCCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCCNC3CCN(C(=O)CCCCCNC(=O)CN4CCN(CC(=O)O)CCN(CC(=O)O)CCN(CC(=O)O)CC4)CC3)CC2)cc1C(C)C] 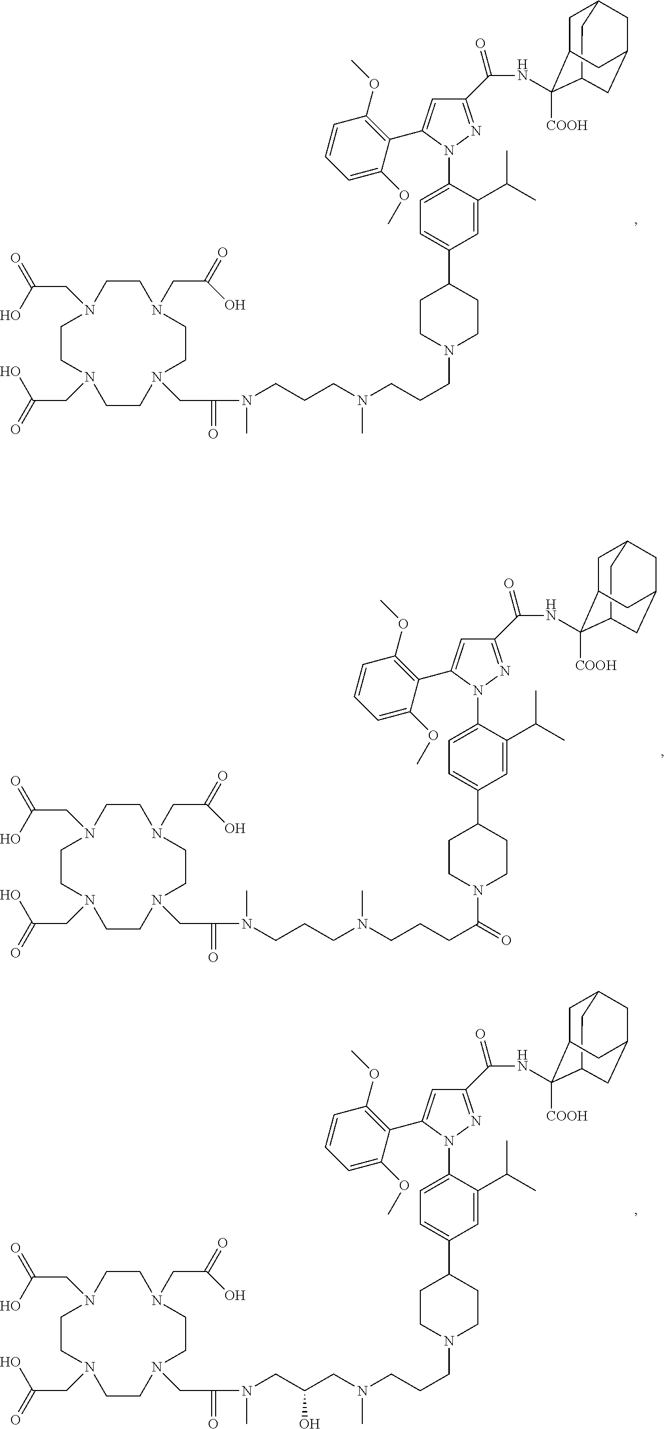 [SMILES: COc1cccc(OC)c1-c1cc(C(=O)NC2(C(=O)O)C3CC4CC(C3)CC2C4)nn1-c1ccc(C2CCN(CCCN(C)CCCN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(C(=O)CCCN(C)CCCN(C)C(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCN(C)C[C@H](O)CN(C)C(=O)CN3CCN(CC(=O)O)CCN(CC(=O)O)CCN(CC(=O)O)CC3)CC2)cc1C(C)C] 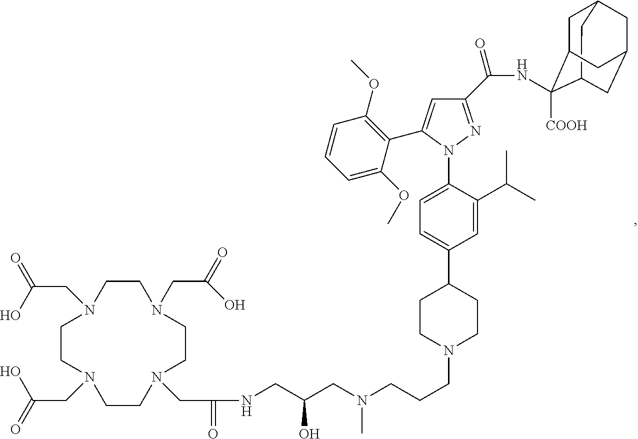 [SMILES: COc1cccc(OC)c1-c1cc(C(=O)NC2(C(=O)O)C3CC4CC(C3)CC2C4)nn1-c1ccc(C2CCN(CCCN(C)C[C@@H](O)CNC(=O)CN3CCN(CC(=O)O)CCN(CC(=O)O)CCN(CC(=O)O)CC3)CC2)cc1C(C)C] 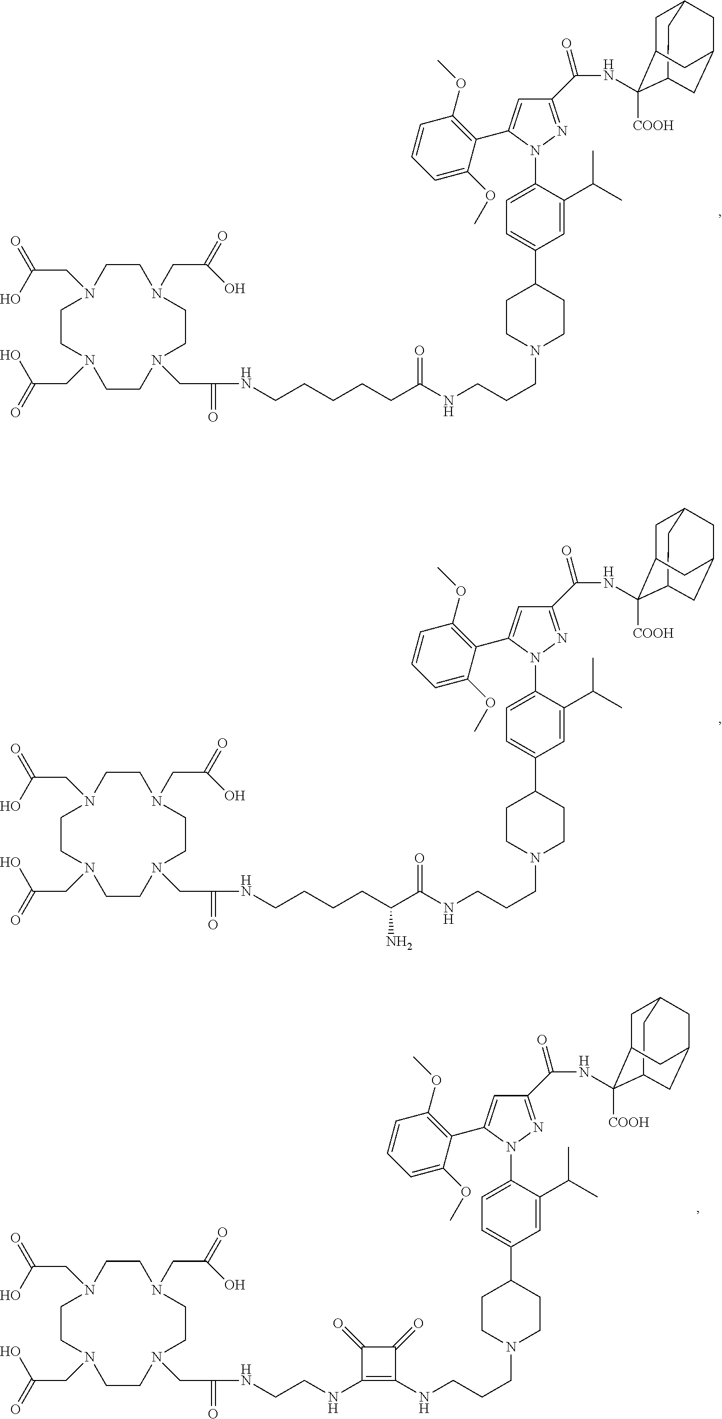 [SMILES: COc1cccc(OC)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)[C@H](N)CCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNc3c(NCCNC(=O)CN4CCN(CC(=O)O)CCN(CC(=O)O)CCN(CC(=O)O)CC4)c(=O)c3=O)CC2)cc1C(C)C] 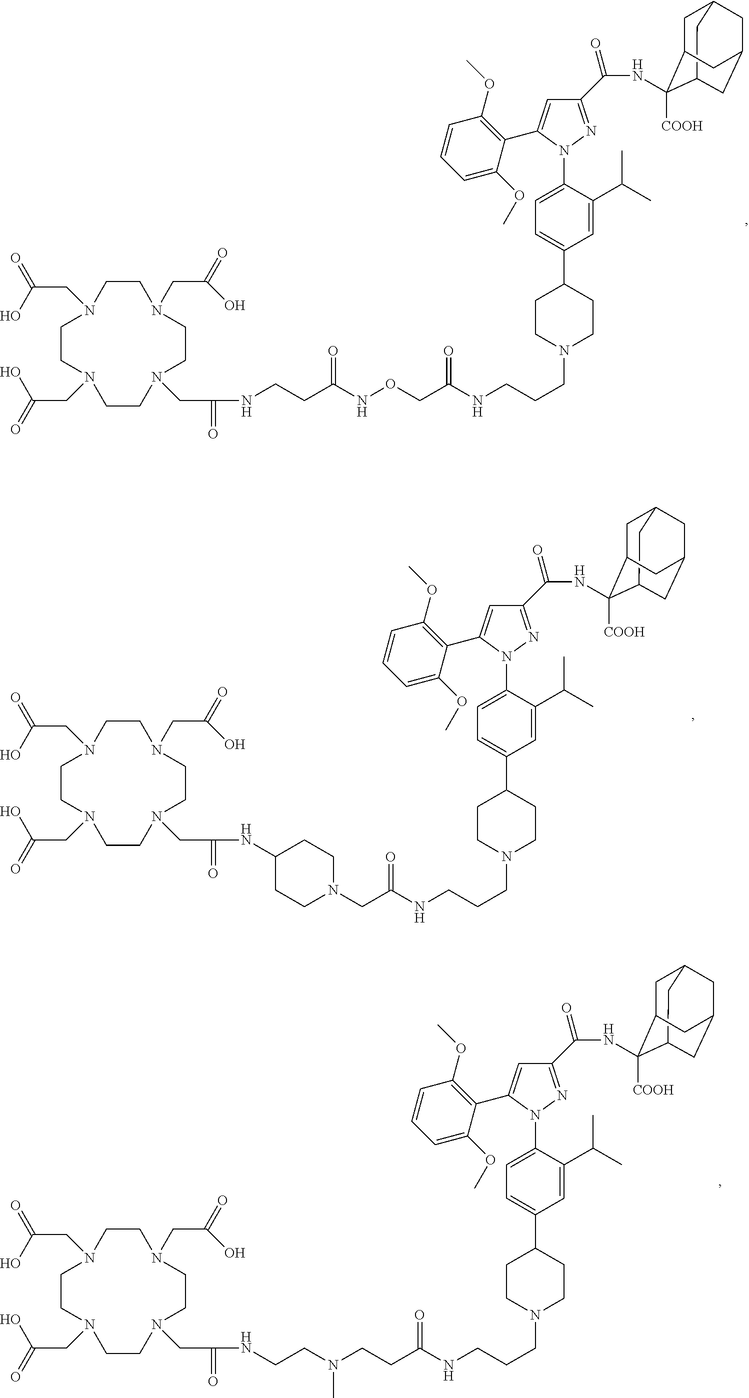 [SMILES: COc1cccc(OC)c1-c1cc(C(=O)NC2(C(=O)O)C3CC4CC(C3)CC2C4)nn1-c1ccc(C2CCN(CCCNC(=O)CONC(=O)CCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CN3CCC(NC(=O)CN4CCN(CC(=O)O)CCN(CC(=O)O)CCN(CC(=O)O)CC4)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CCN(C)CCNC(=O)CN3CCN(CC(=O)O)CCN(CC(=O)O)CCN(CC(=O)O)CC3)CC2)cc1C(C)C] 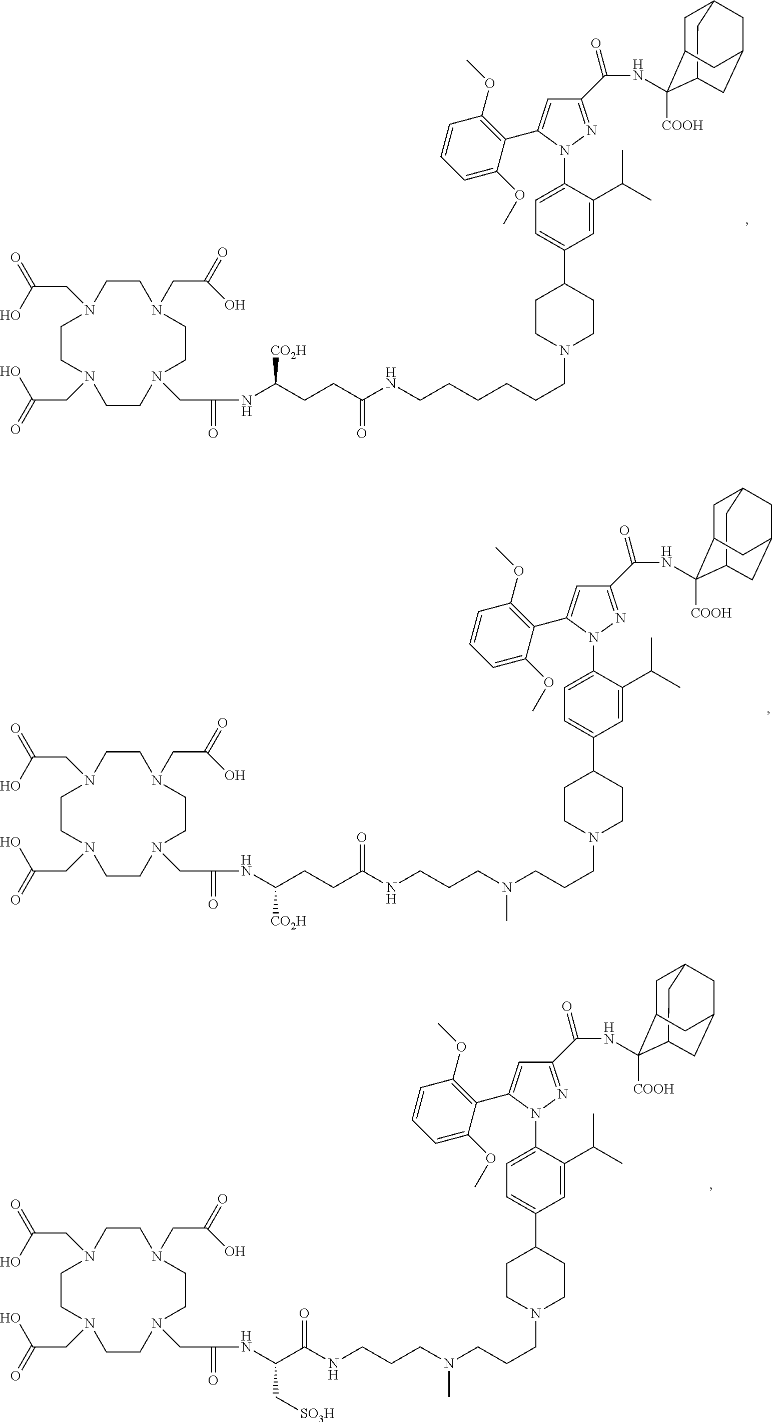 [SMILES: COc1cccc(OC)c1-c1cc(C(=O)NC2(C(=O)O)C3CC4CC(C3)CC2C4)nn1-c1ccc(C2CCN(CCCCCCNC(=O)CC[C@@H](NC(=O)CN3CCN(CC(=O)O)CCN(CC(=O)O)CCN(CC(=O)O)CC3)C(=O)O)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)CC[C@@H](NC(=O)CN3CCN(CC(=O)O)CCN(CC(=O)O)CCN(CC(=O)O)CC3)C(=O)O)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C] 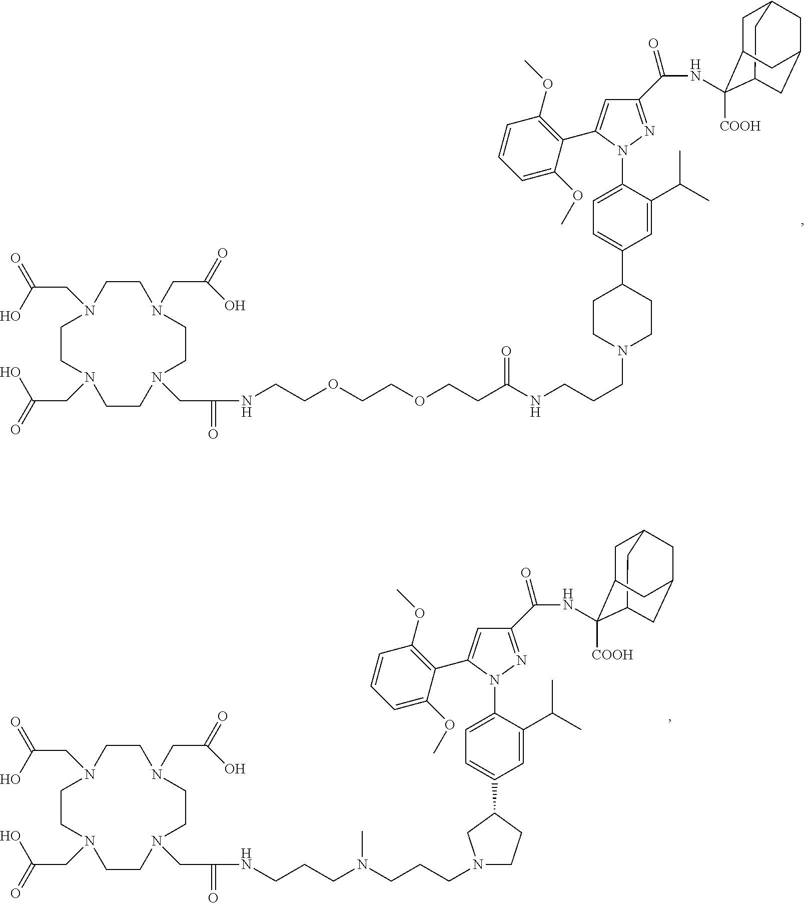 [SMILES: COc1cccc(OC)c1-c1cc(C(=O)NC2(C(=O)O)C3CC4CC(C3)CC2C4)nn1-c1ccc(C2CCN(CCCNC(=O)CCOCCOCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc([C@@H]2CCN(CCCN(C)CCCNC(=O)CN3CCN(CC(=O)O)CCN(CC(=O)O)CCN(CC(=O)O)CC3)C2)cc1C(C)C] 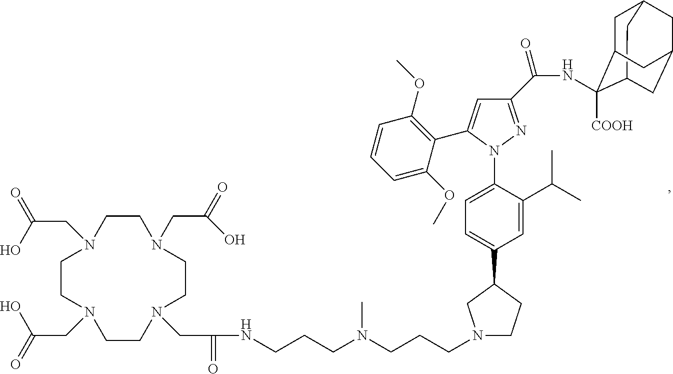 [SMILES: COc1cccc(OC)c1-c1cc(C(=O)NC2(C(=O)O)C3CC4CC(C3)CC2C4)nn1-c1ccc([C@H]2CCN(CCCN(C)CCCNC(=O)CN3CCN(CC(=O)O)CCN(CC(=O)O)CCN(CC(=O)O)CC3)C2)cc1C(C)C] 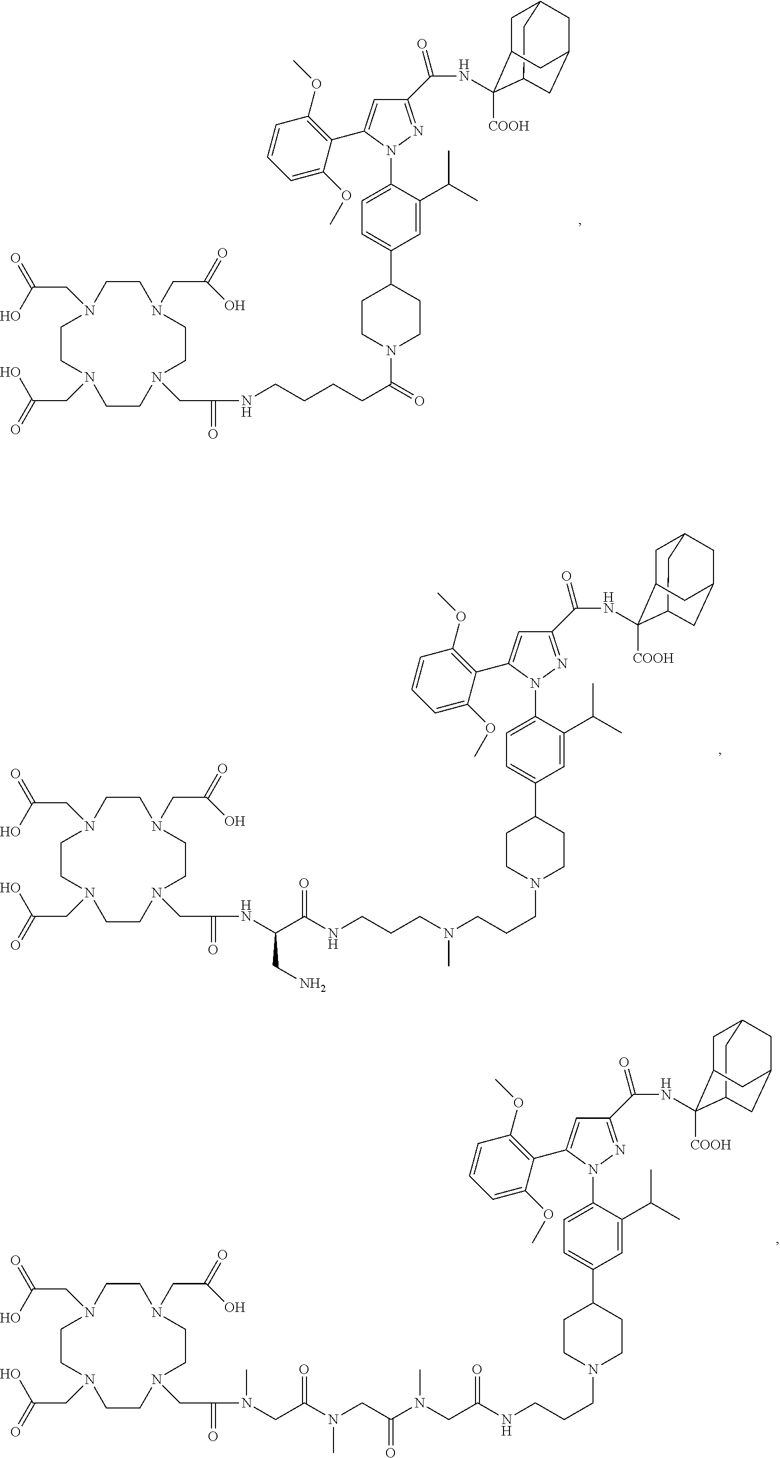 [SMILES: COc1cccc(OC)c1-c1cc(C(=O)NC2(C(=O)O)C3CC4CC(C3)CC2C4)nn1-c1ccc(C2CCN(C(=O)CCCCNC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCN(C)CCCNC(=O)[C@@H](CN)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCNC(=O)CN(C)C(=O)CN(C)C(=O)CN(C)C(=O)CN3CCN(CC(=O)O)CCN(CC(=O)O)CCN(CC(=O)O)CC3)CC2)cc1C(C)C] 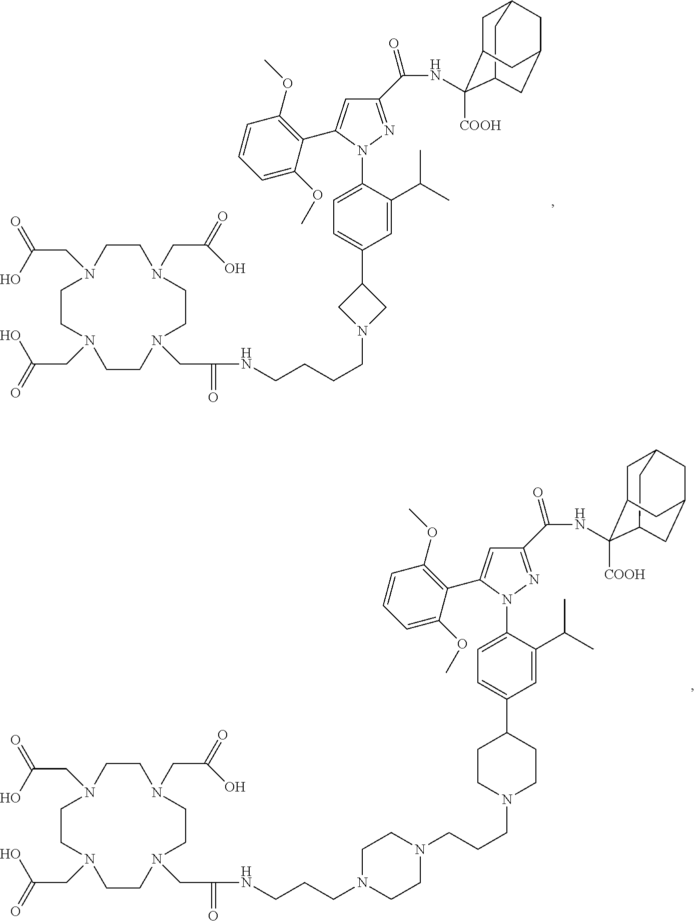 [SMILES: COc1cccc(OC)c1-c1cc(C(=O)NC2(C(=O)O)C3CC4CC(C3)CC2C4)nn1-c1ccc(C2CN(CCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCN3CCN(CCCNC(=O)CN4CCN(CC(=O)O)CCN(CC(=O)O)CCN(CC(=O)O)CC4)CC3)CC2)cc1C(C)C] 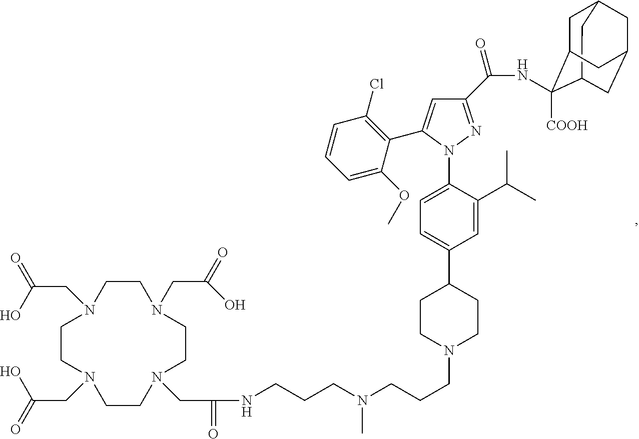 [SMILES: COc1cccc(Cl)c1-c1cc(C(=O)NC2(C(=O)O)C3CC4CC(C3)CC2C4)nn1-c1ccc(C2CCN(CCCN(C)CCCNC(=O)CN3CCN(CC(=O)O)CCN(CC(=O)O)CCN(CC(=O)O)CC3)CC2)cc1C(C)C] 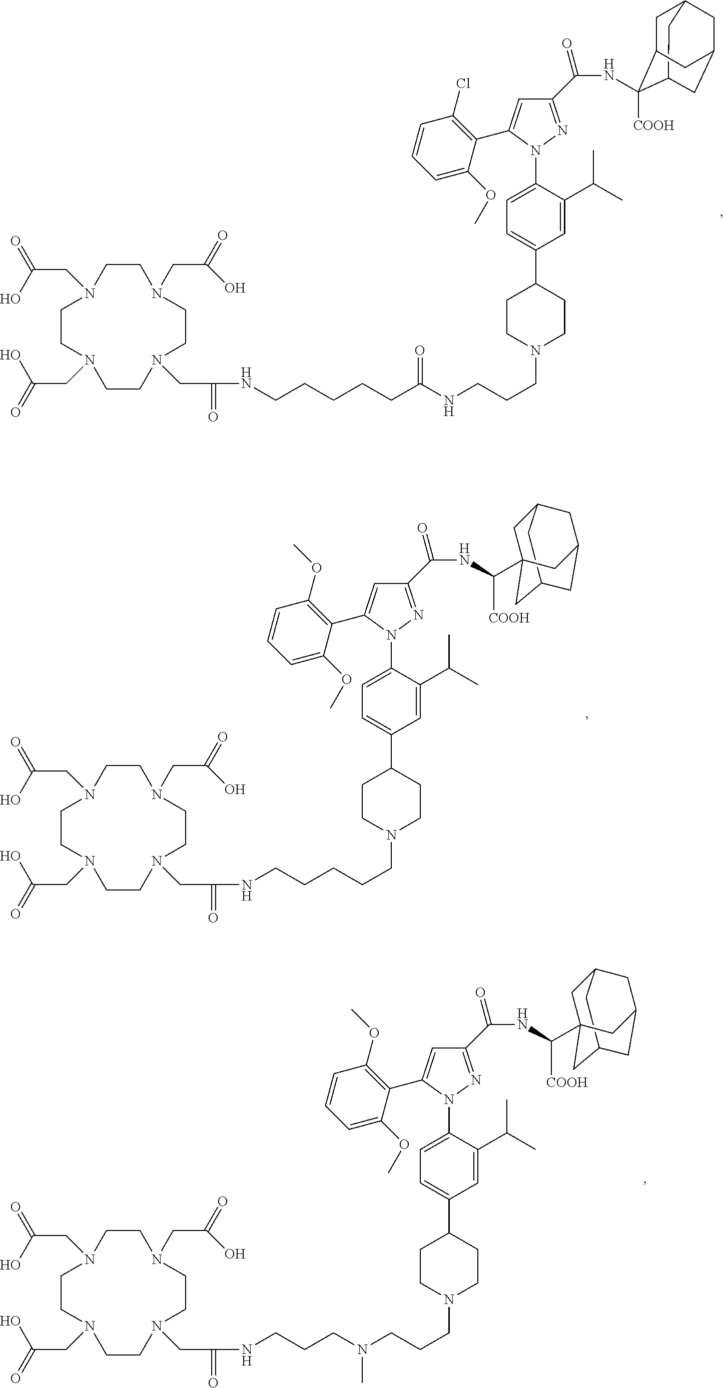 [SMILES: COc1cccc(Cl)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2CCN(CCCCCNC(=O)CN3CCN(CC(=O)O)CCN(CC(=O)O)CCN(CC(=O)O)CC3)CC2)cc1C(C)C ; COc1cccc(OC)c1-c1cc(C(=O)N[C@H](C(=O)O)C23CC4CC(CC(C4)C2)C3)nn1-c1ccc(C2CCN(CCCN(C)CCCNC(=O)CN3CCN(CC(=O)O)CCN(CC(=O)O)CCN(CC(=O)O)CC3)CC2)cc1C(C)C] 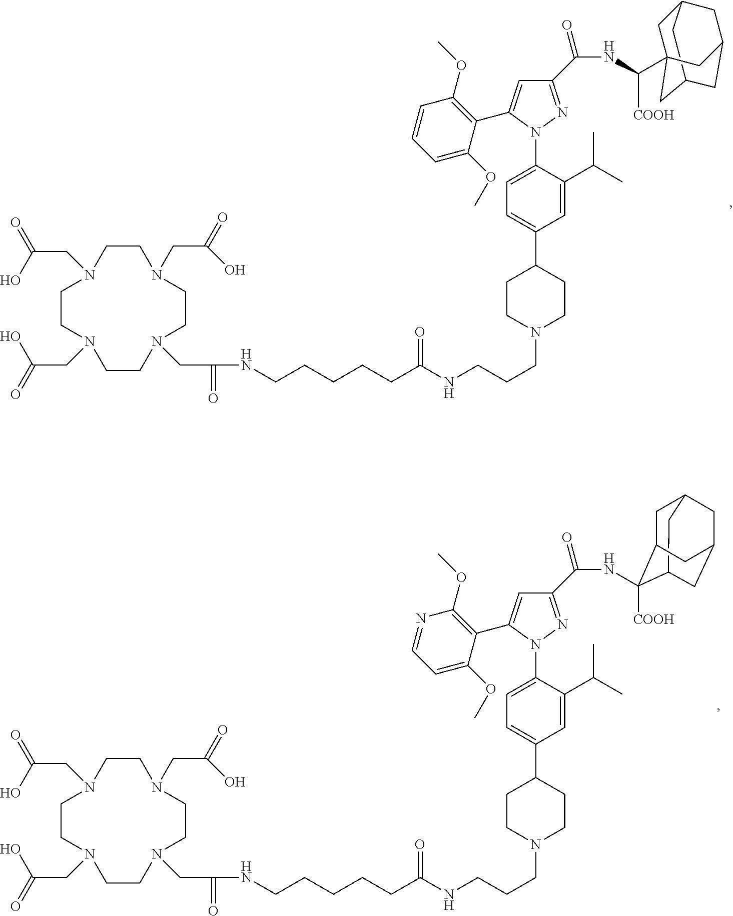 [SMILES: COc1cccc(OC)c1-c1cc(C(=O)N[C@H](C(=O)O)C23CC4CC(CC(C4)C2)C3)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C] 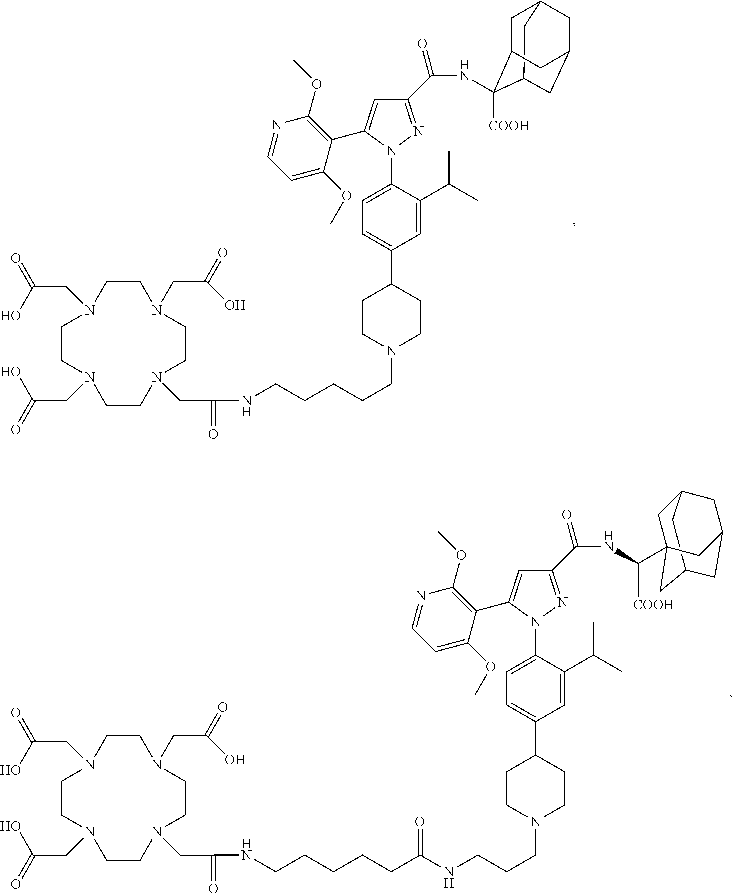 [SMILES: COc1ccnc(OC)c1-c1cc(C(=O)NC2(C(=O)O)C3CC4CC(C3)CC2C4)nn1-c1ccc(C2CCN(CCCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)N[C@H](C(=O)O)C23CC4CC(CC(C4)C2)C3)nn1-c1ccc(C2CCN(CCCNC(=O)CCCCCNC(=O)CN3CCN(CC(=O)O)CCN(CC(=O)O)CCN(CC(=O)O)CC3)CC2)cc1C(C)C] 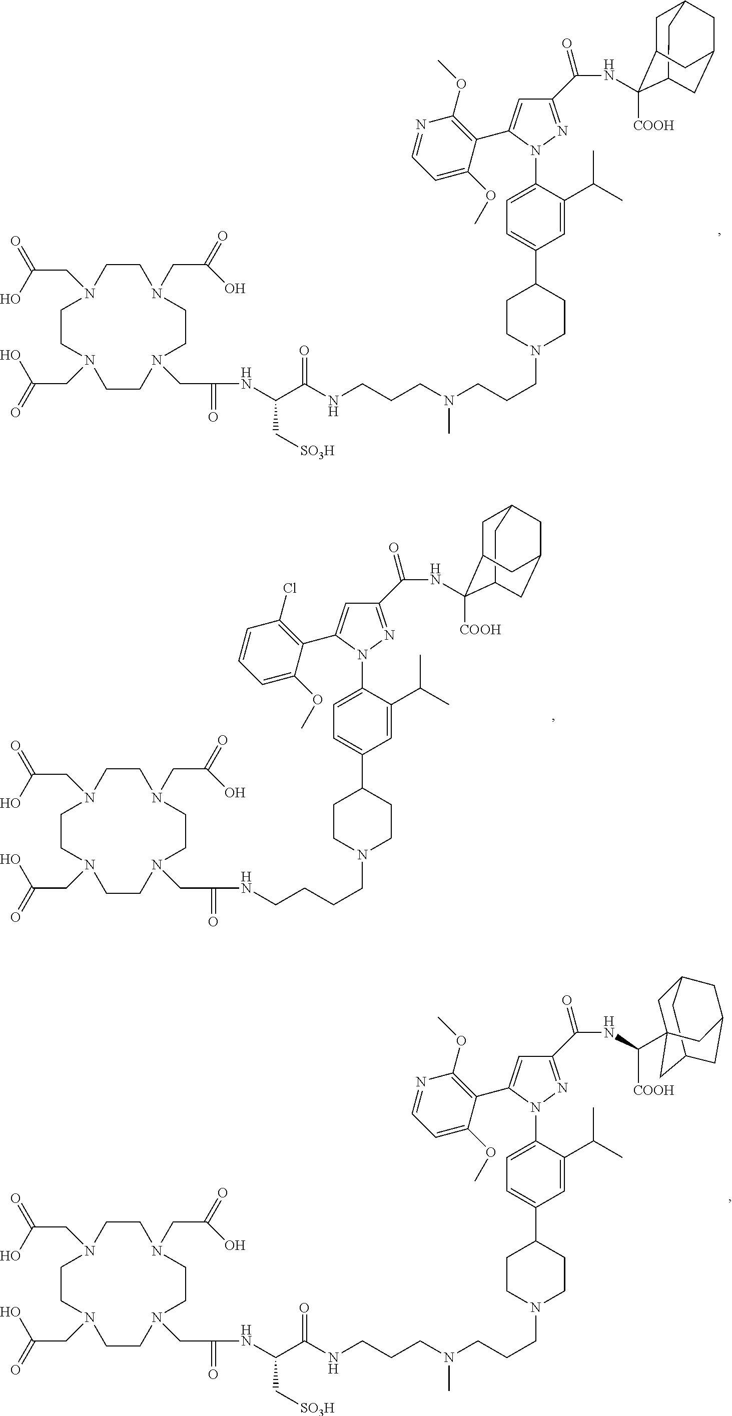 [SMILES: COc1ccnc(OC)c1-c1cc(C(=O)NC2(C(=O)O)C3CC4CC(C3)CC2C4)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C ; COc1cccc(Cl)c1-c1cc(C(=O)NC2(C(=O)O)C3CC4CC(C3)CC2C4)nn1-c1ccc(C2CCN(CCCCNC(=O)CN3CCN(CC(=O)O)CCN(CC(=O)O)CCN(CC(=O)O)CC3)CC2)cc1C(C)C ; COc1ccnc(OC)c1-c1cc(C(=O)N[C@H](C(=O)O)C23CC4CC(CC(C4)C2)C3)nn1-c1ccc(C2CCN(CCCN(C)CCCNC(=O)[C@H](CS(=O)(=O)O)NC(=O)CN3CCN(CC(=O)O)CCN(CC(=O)O)CCN(CC(=O)O)CC3)CC2)cc1C(C)C] 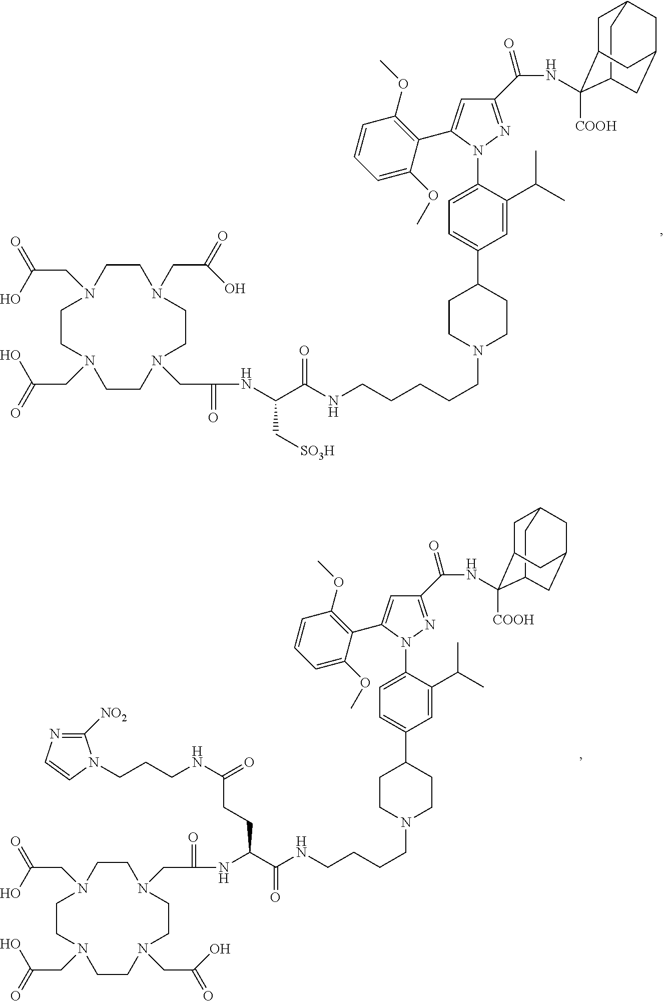 [SMILES: COc1cccc(OC)c1-c1cc(C(=O)NC2(C(=O)O)C3CC4CC(C3)CC2C4)nn1-c1ccc(C2CCN(CCCCCNC(=O)[C@H](CS(=O)(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)CC2)cc1C(C)C] 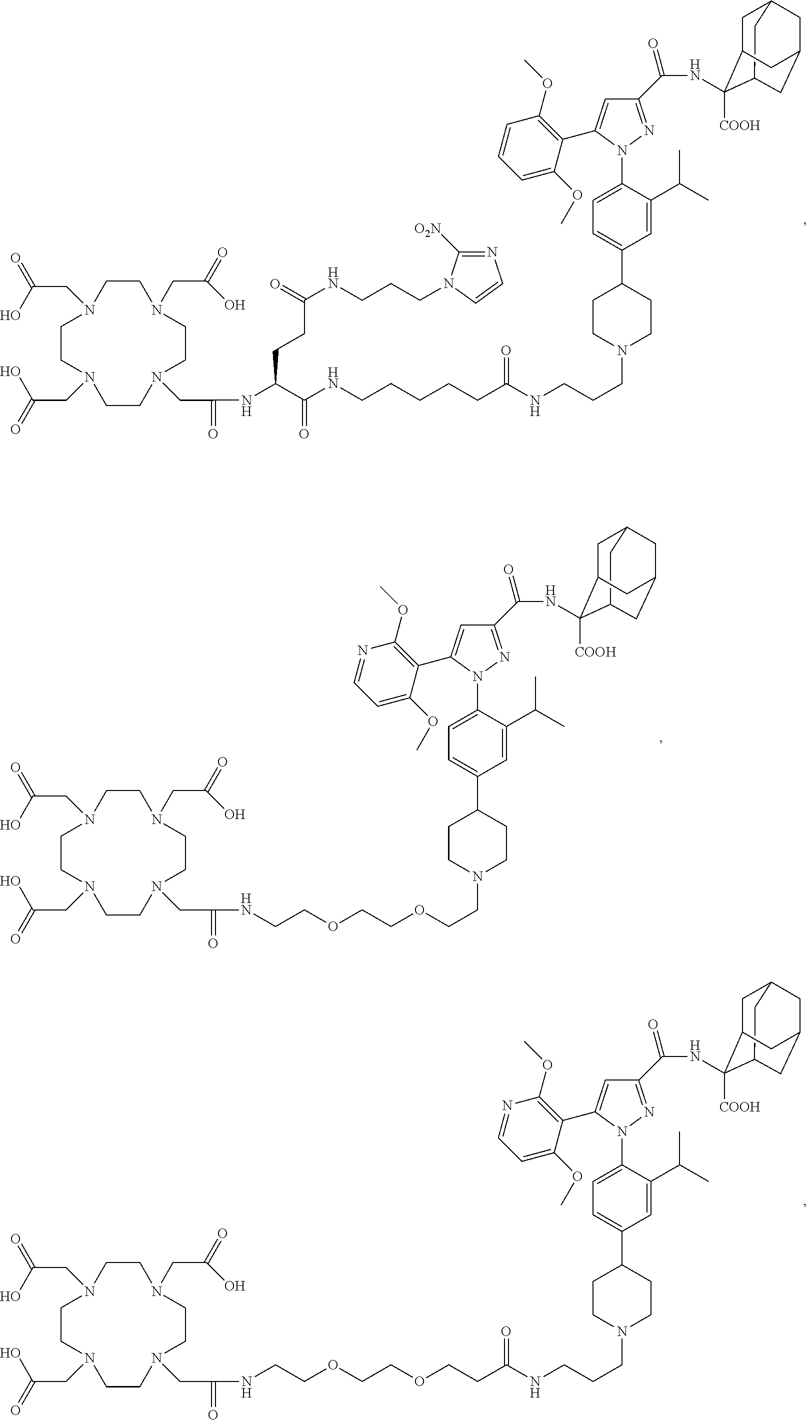 [SMILES: COc1cccc(OC)c1-c1cc(C(=O)NC2(C(=O)O)C3CC4CC(C3)CC2C4)nn1-c1ccc(C2CCN(CCCNC(=O)CCCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCOCCOCCNC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCNC(=O)CCOCCOCCNC(=O)CN3CCN(CC(=O)O)CCN(CC(=O)O)CCN(CC(=O)O)CC3)CC2)cc1C(C)C] 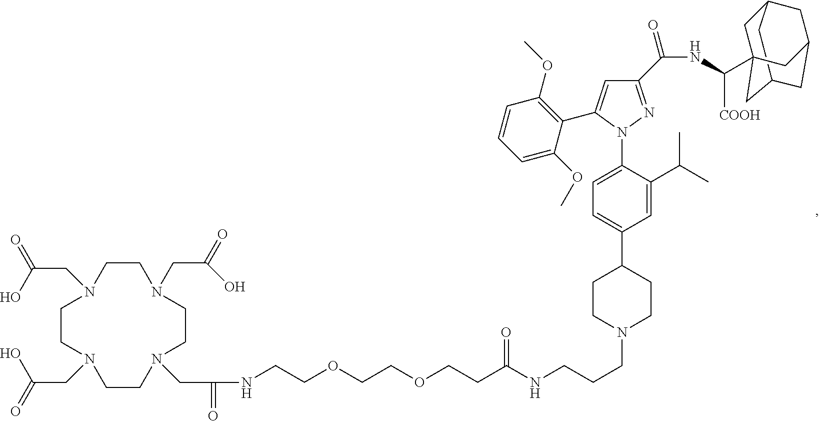 [SMILES: COc1cccc(OC)c1-c1cc(C(=O)N[C@H](C(=O)O)C23CC4CC(CC(C4)C2)C3)nn1-c1ccc(C2CCN(CCCNC(=O)CCOCCOCCNC(=O)CN3CCN(CC(=O)O)CCN(CC(=O)O)CCN(CC(=O)O)CC3)CC2)cc1C(C)C] 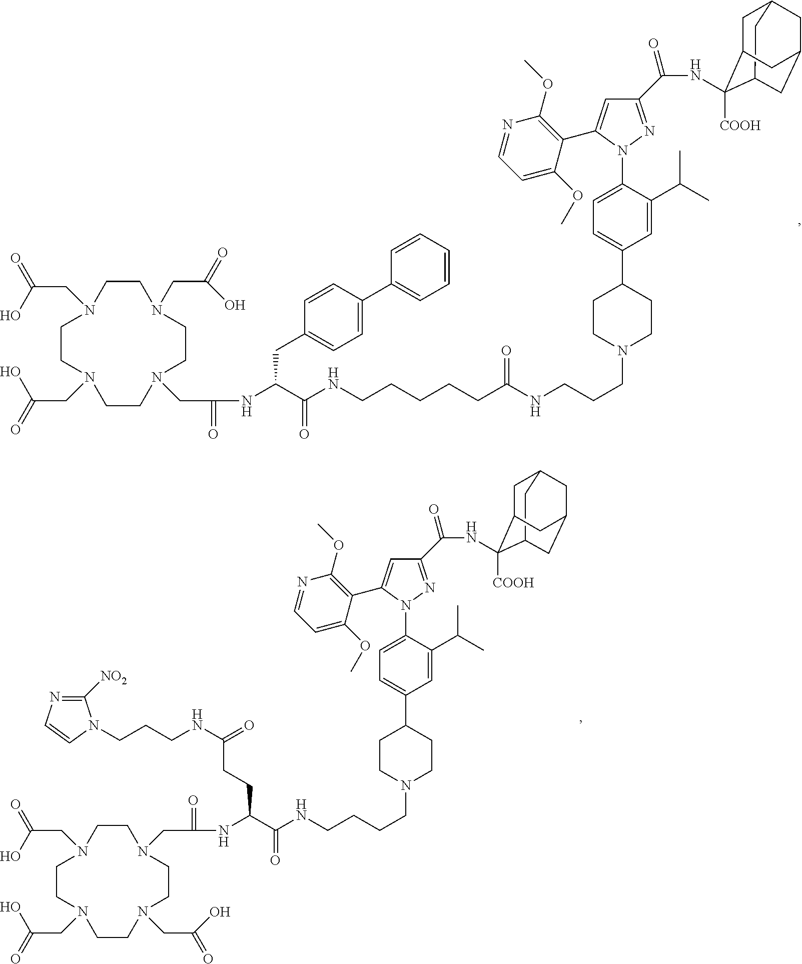 [SMILES: COc1ccnc(OC)c1-c1cc(C(=O)NC2(C(=O)O)C3CC4CC(C3)CC2C4)nn1-c1ccc(C2CCN(CCCNC(=O)CCCCCNC(=O)[C@@H](Cc3ccc(-c4ccccc4)cc3)NC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)CC2)cc1C(C)C] 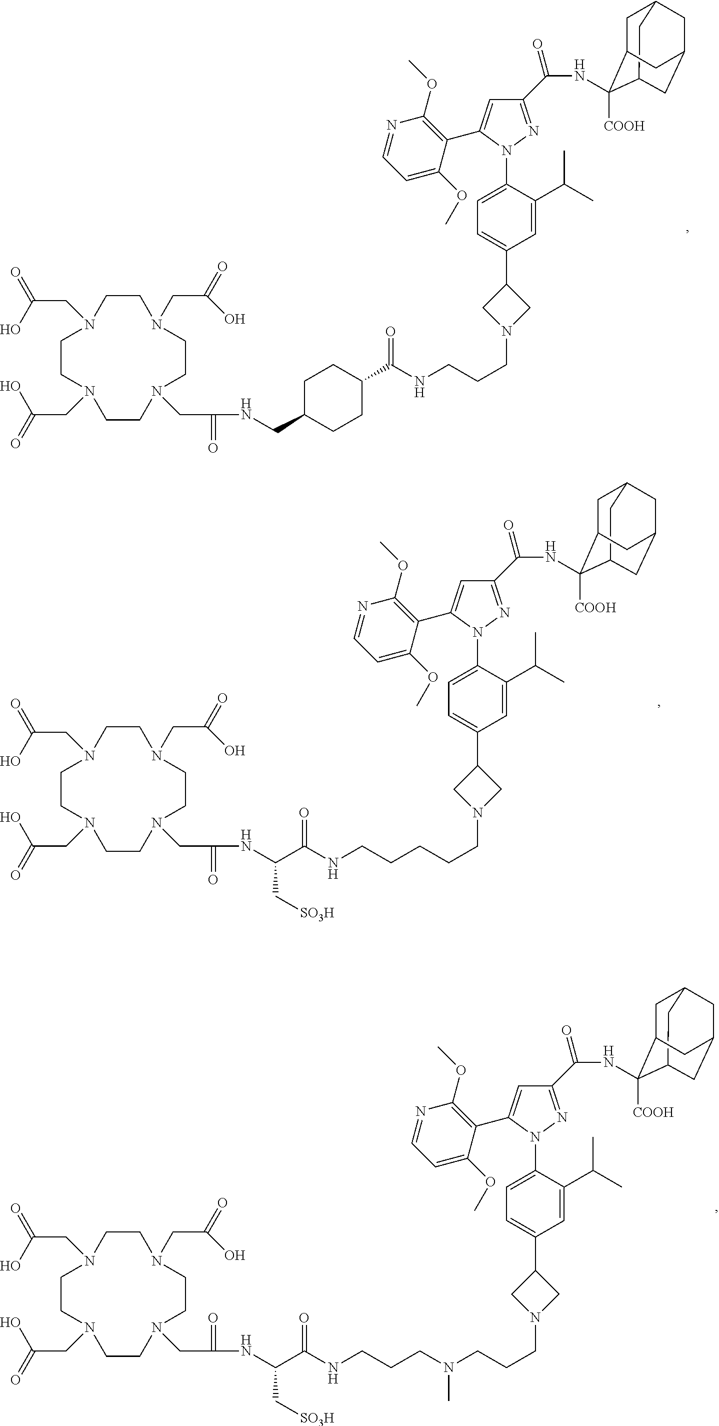 [SMILES: COc1ccnc(OC)c1-c1cc(C(=O)NC2(C(=O)O)C3CC4CC(C3)CC2C4)nn1-c1ccc(C2CN(CCCNC(=O)[C@H]3CC[C@H](CNC(=O)CN4CCN(CC(=O)O)CCN(CC(=O)O)CCN(CC(=O)O)CC4)CC3)C2)cc1C(C)C ; COc1ccnc(OC)c1-c1cc(C(=O)NC2(C(=O)O)C3CC4CC(C3)CC2C4)nn1-c1ccc(C2CN(CCCCCNC(=O)[C@H](CS(=O)(=O)O)NC(=O)CN3CCN(CC(=O)O)CCN(CC(=O)O)CCN(CC(=O)O)CC3)C2)cc1C(C)C ; COc1ccnc(OC)c1-c1cc(C(=O)NC2(C(=O)O)C3CC4CC(C3)CC2C4)nn1-c1ccc(C2CN(CCCN(C)CCCNC(=O)[C@H](CS(=O)(=O)O)NC(=O)CN3CCN(CC(=O)O)CCN(CC(=O)O)CCN(CC(=O)O)CC3)C2)cc1C(C)C] 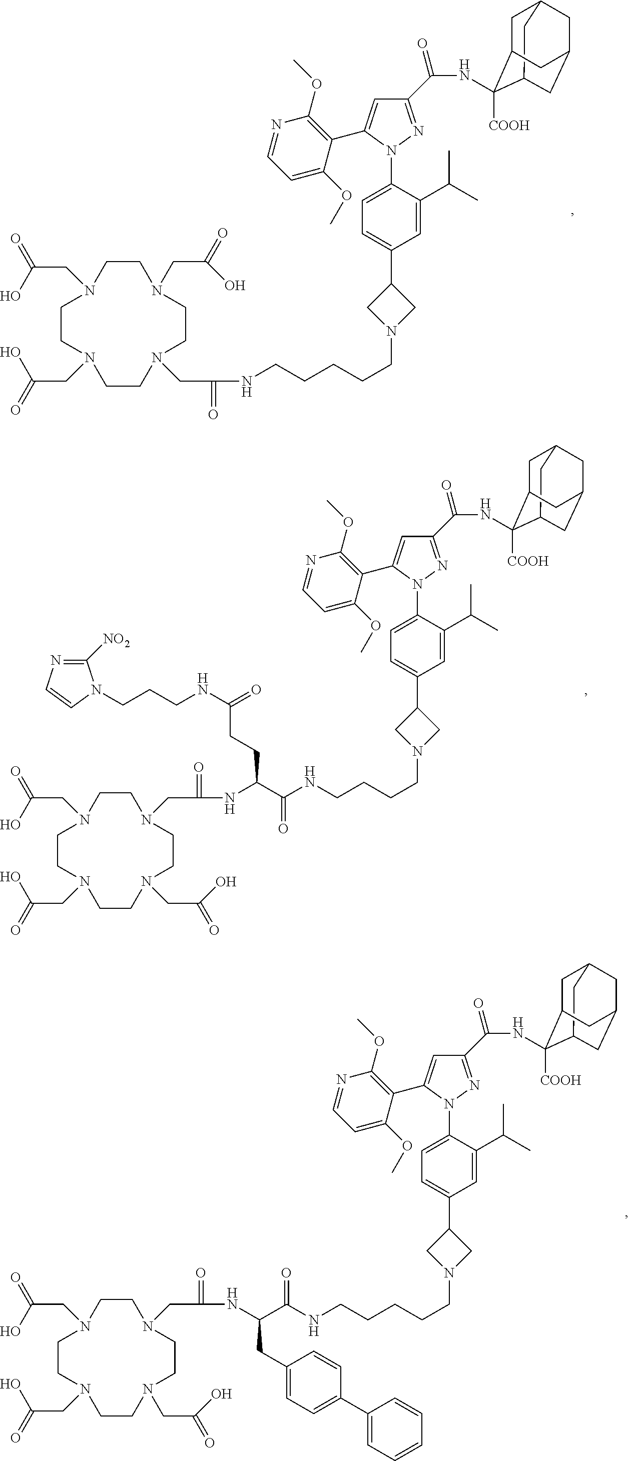 [SMILES: COc1ccnc(OC)c1-c1cc(C(=O)NC2(C(=O)O)C3CC4CC(C3)CC2C4)nn1-c1ccc(C2CN(CCCCCNC(=O)CN3CCN(CC(=O)O)CCN(CC(=O)O)CCN(CC(=O)O)CC3)C2)cc1C(C)C ; COc1ccnc(OC)c1-c1cc(C(=O)NC2(C(=O)O)C3CC4CC(C3)CC2C4)nn1-c1ccc(C2CN(CCCCNC(=O)[C@H](CCC(=O)NCCCn3ccnc3[N+](=O)[O-])NC(=O)CN3CCN(CC(=O)O)CCN(CC(=O)O)CCN(CC(=O)O)CC3)C2)cc1C(C)C ; COc1ccnc(OC)c1-c1cc(C(=O)NC2(C(=O)O)C3CC4CC(C3)CC2C4)nn1-c1ccc(C2CN(CCCCCNC(=O)[C@@H](Cc3ccc(-c4ccccc4)cc3)NC(=O)CN3CCN(CC(=O)O)CCN(CC(=O)O)CCN(CC(=O)O)CC3)C2)cc1C(C)C] 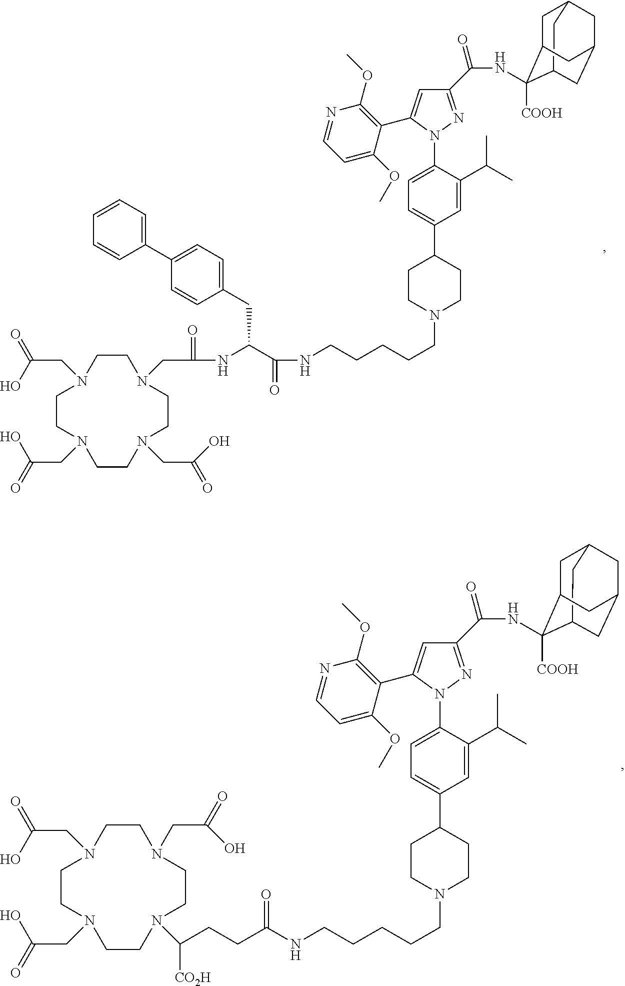 [SMILES: COc1ccnc(OC)c1-c1cc(C(=O)NC2(C(=O)O)C3CC4CC(C3)CC2C4)nn1-c1ccc(C2CCN(CCCCCNC(=O)[C@@H](Cc3ccc(-c4ccccc4)cc3)NC(=O)CN3CCN(CC(=O)O)CCN(CC(=O)O)CCN(CC(=O)O)CC3)CC2)cc1C(C)C ; COc1ccnc(OC)c1-c1cc(C(=O)NC2(C(=O)O)C3CC4CC(C3)CC2C4)nn1-c1ccc(C2CCN(CCCCCNC(=O)CCC(C(=O)O)N3CCN(CC(=O)O)CCN(CC(=O)O)CCN(CC(=O)O)CC3)CC2)cc1C(C)C] 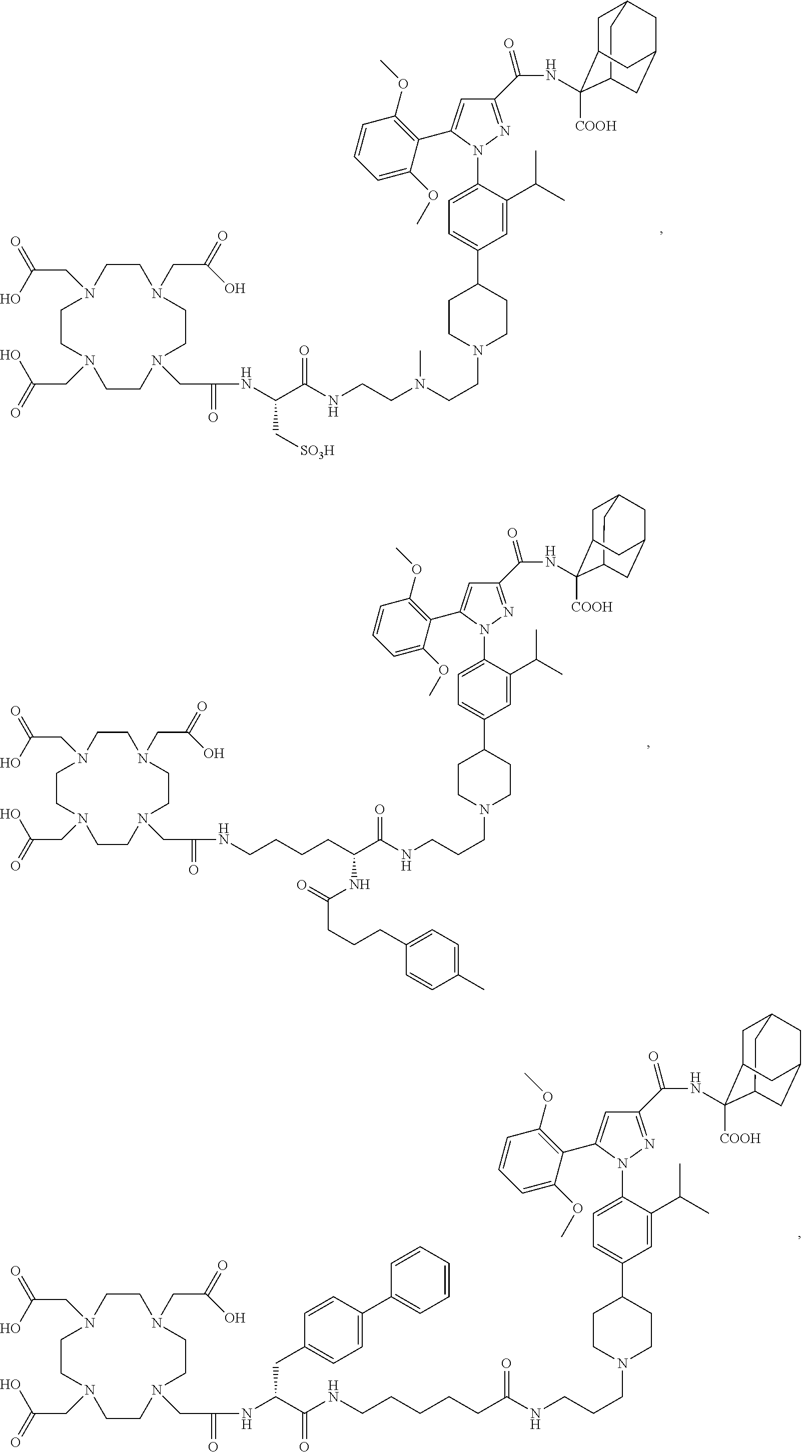 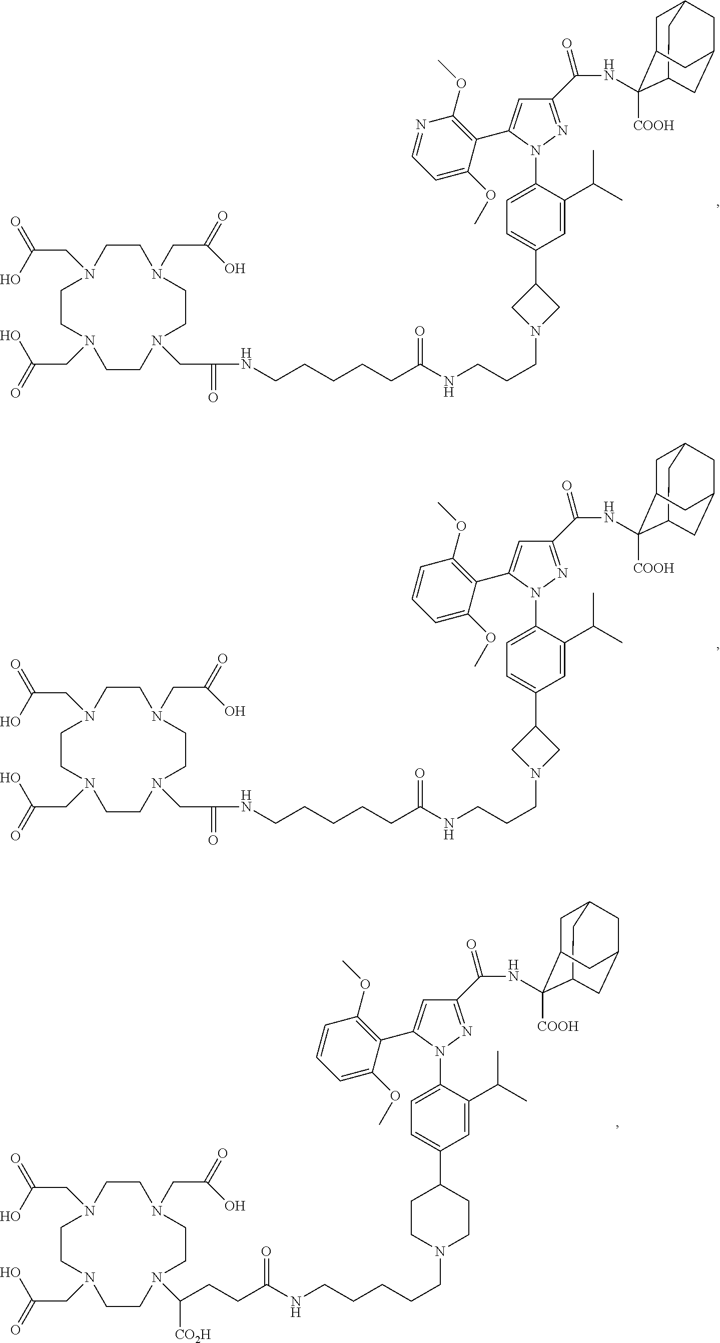 [SMILES: COc1ccnc(OC)c1-c1cc(C(=O)NC2(C(=O)O)C3CC4CC(C3)CC2C4)nn1-c1ccc(C2CN(CCCNC(=O)CCCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CN(CCCNC(=O)CCCCCNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCCCNC(=O)CCC(C(=O)O)N3CCN(CC(=O)O)CCN(CC(=O)O)CCN(CC(=O)O)CC3)CC2)cc1C(C)C] 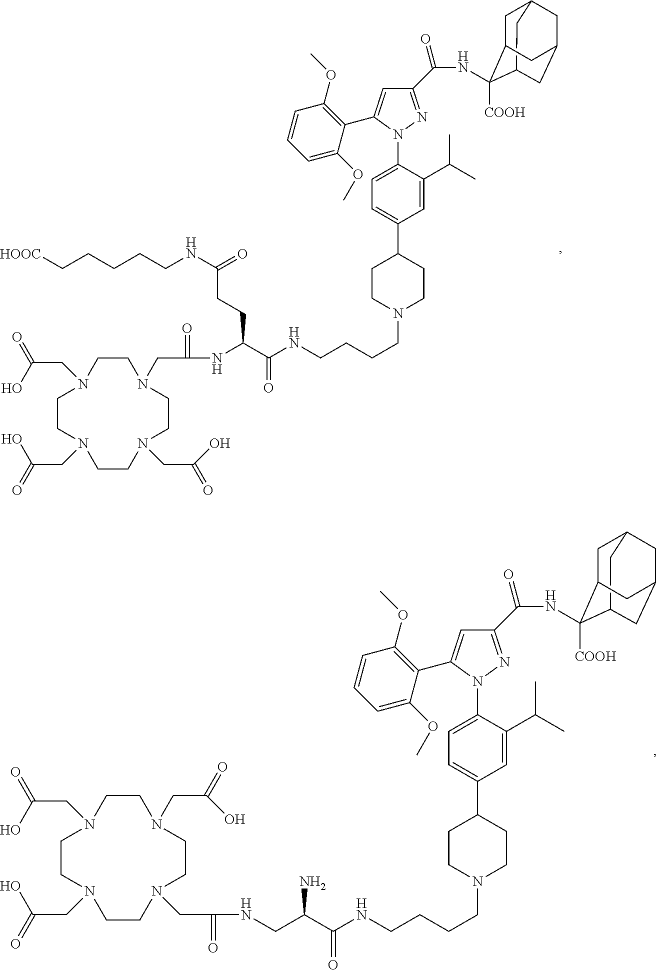 [SMILES: COc1cccc(OC)c1-c1cc(C(=O)NC2(C(=O)O)C3CC4CC(C3)CC2C4)nn1-c1ccc(C2CCN(CCCCNC(=O)[C@H](CCC(=O)NCCCCCC(=O)O)NC(=O)CN3CCN(CC(=O)O)CCN(CC(=O)O)CCN(CC(=O)O)CC3)CC2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](N)CNC(=O)CN3CCN(CC(=O)O)CCN(CC(=O)O)CCN(CC(=O)O)CC3)CC2)cc1C(C)C] 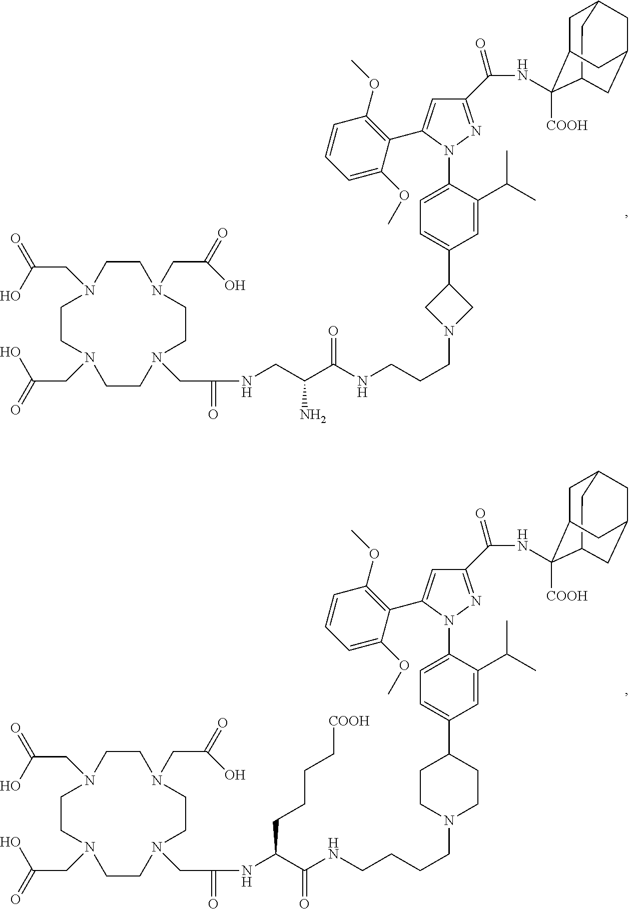 [SMILES: COc1cccc(OC)c1-c1cc(C(=O)NC2(C(=O)O)C3CC4CC(C3)CC2C4)nn1-c1ccc(C2CN(CCCNC(=O)[C@H](N)CNC(=O)CN3CCN(CC(=O)O)CCN(CC(=O)O)CCN(CC(=O)O)CC3)C2)cc1C(C)C ; COc1cccc(OC)c1-c1cc(C(=O)NC2(C(=O)O)C3CC4CC(C3)CC2C4)nn1-c1ccc(C2CCN(CCCCNC(=O)[C@H](CCCCC(=O)O)NC(=O)CN3CCN(CC(=O)O)CCN(CC(=O)O)CCN(CC(=O)O)CC3)CC2)cc1C(C)C] 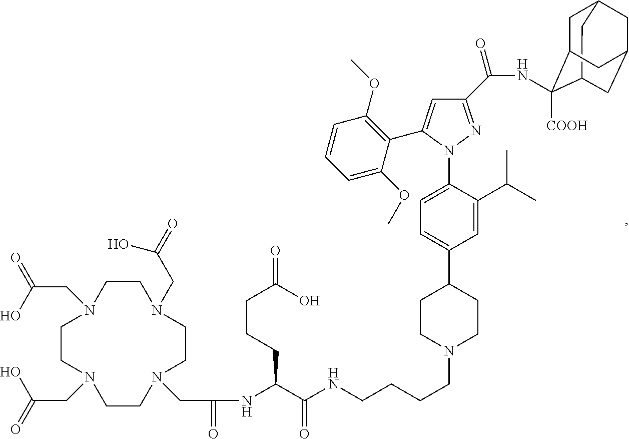 [SMILES: COc1cccc(OC)c1-c1cc(C(=O)NC2(C(=O)O)C3CC4CC(C3)CC2C4)nn1-c1ccc(C2CCN(CCCCNC(=O)[C@H](CCCC(=O)O)NC(=O)CN3CCN(CC(=O)O)CCN(CC(=O)O)CCN(CC(=O)O)CC3)CC2)cc1C(C)C] 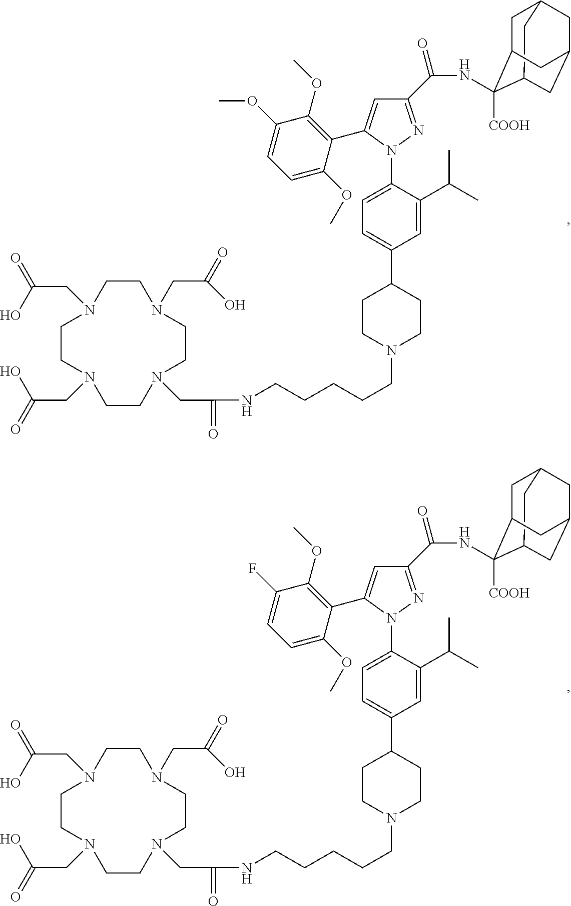 [SMILES: COc1ccc(OC)c(-c2cc(C(=O)NC3(C(=O)O)C4CC5CC(C4)CC3C5)nn2-c2ccc(C3CCN(CCCCCNC(=O)CN4CCN(CC(=O)O)CCN(CC(=O)O)CCN(CC(=O)O)CC4)CC3)cc2C(C)C)c1OC ; COc1ccc(F)c(OC)c1-c1cc(C(=O)NC2(C(=O)O)C3CC4CC(C3)CC2C4)nn1-c1ccc(C2CCN(CCCCCNC(=O)CN3CCN(CC(=O)O)CCN(CC(=O)O)CCN(CC(=O)O)CC3)CC2)cc1C(C)C] 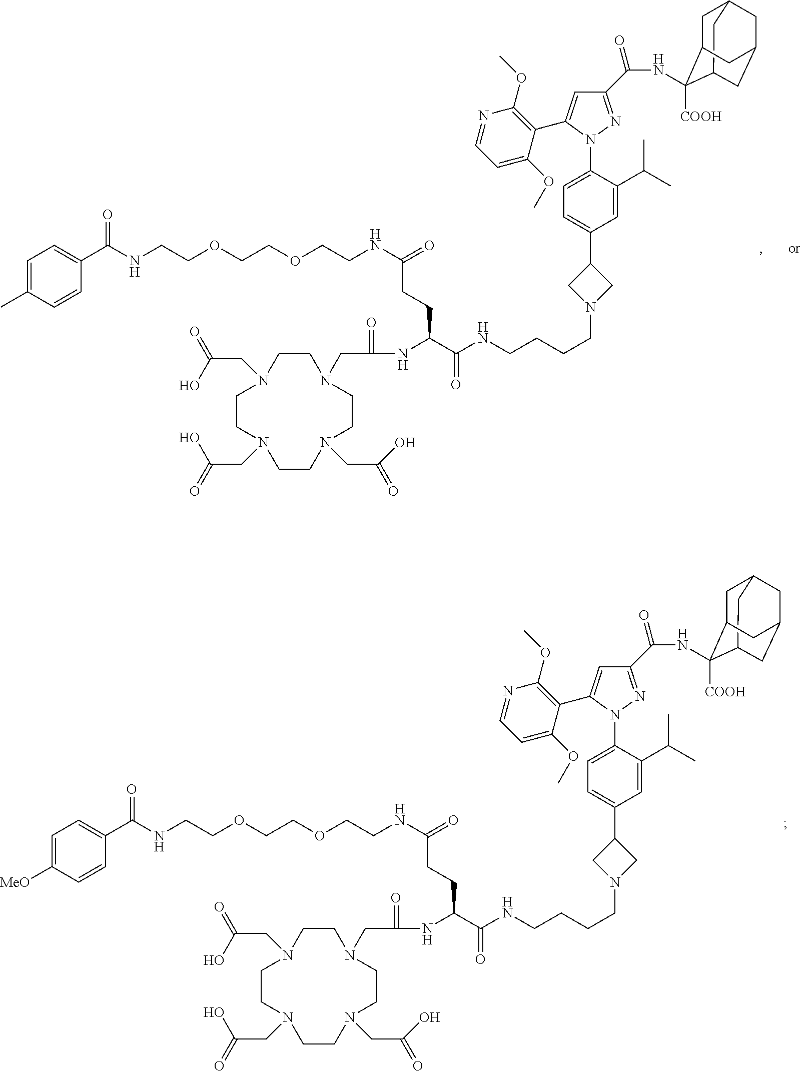 [SMILES: COc1ccnc(OC)c1-c1cc(C(=O)NC2(C(=O)O)C3CC4CC(C3)CC2C4)nn1-c1ccc(C2CN(CCCCNC(=O)[C@H](CCC(=O)NCCOCCOCCNC(=O)c3ccc(C)cc3)NC(=O)CN3CCN(CC(=O)O)CCN(CC(=O)O)CCN(CC(=O)O)CC3)C2)cc1C(C)C ; COc1ccc(C(=O)NCCOCCOCCNC(=O)CC[C@H](NC(=O)CN2CCN(CC(=O)O)CCN(CC(=O)O)CCN(CC(=O)O)CC2)C(=O)NCCCCN2CC(c3ccc(-n4nc(C(=O)NC5(C(=O)O)C6CC7CC(C6)CC5C7)cc4-c4c(OC)ccnc4OC)c(C(C)C)c3)C2)cc1]
or a radionuclide complex thereof.

22. The compound of claim 1, or a pharmaceutically acceptable salt thereof, wherein the compound is a radionuclide complex and the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (⁹⁹mTc), or 195m-platinum (^{195m}pt); or
an α-emitting radionuclide that is 225-actinium (²²⁵Ac), 213-bismuth (²¹³Bi), 223-radium (²²³Ra), or 212-lead (²¹²Pb); or
a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or
a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

23. The compound of claim 1, or a pharmaceutically acceptable salt thereof, wherein: the compound is a radionuclide complex and the radionuclide of the radionuclide complex is 111-indium (¹¹¹In), 67-gallium (⁶⁷Ga), 68-gallium (⁶⁸Ga), 69-gallium (⁶⁹Ga), 71-gallium (⁷¹Ta), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

24. The compound of claim 1, or a pharmaceutically acceptable salt thereof, wherein: the compound is a radionuclide complex and the radionuclide of the radionuclide complex is 64-copper (⁶⁴Cu), 67-copper (⁶⁷Cu), 90-yttrium (⁹⁰Y), 111-indium (¹¹¹In), 67-gallium (⁶⁷Ga), 68-gallium (⁶⁸Ga), 225-actinium (²²⁵Ac), or 177-lutetium (¹⁷⁷Lu) or 212-lead (²¹²Pb).

25. A pharmaceutical composition comprising a compound of claim 1, or a pharmaceutically acceptable salt thereof, and at least one pharmaceutically acceptable excipient.

26. A method for the treatment of cancer comprising administering to a mammal with cancer an effective amount of a compound of claim 1, or a pharmaceutically acceptable salt thereof; wherein the cancer comprises tumors and the tumors overexpress Neurotensin Receptor 1 (NTSR1); and wherein the compound is a radionuclide complex and the radionuclide of the radionuclide complex is:
111-indium (¹¹¹In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

27. The method of claim 26, wherein the cancer is lung cancer, pancreatic cancer, colorectal cancer, head and neck cancers, breast cancer, prostate cancer, gastrointestinal cancer, or bone cancer.

28. A method of killing tumors in a mammal that overexpress Neurotensin Receptor 1 (NTSR1) comprising administering to the mammal a compound of claim 1, or a pharmaceutically acceptable salt thereof, wherein the compound is a radionuclide complex and the radionuclide of the radionuclide complex is:
111-indium (¹¹¹In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

29. The method of claim 28, wherein the mammal has been diagnosed with lung cancer, pancreatic cancer, colorectal cancer, head and neck cancers, breast cancer, prostate cancer, gastrointestinal cancer, or bone cancer.

30. A method for the in vivo imaging of tissues or organs in a mammal with tumors expressing Neurotensin Receptor 1 (NTSR1) comprising administering to the mammal a compound of claim 1, or a pharmaceutically acceptable salt thereof; and performing positron emission tomography (PET) analysis or single-photon emission computerized tomography (SPECT); wherein the compound of claim 1, or a pharmaceutically acceptable salt thereof, comprises a diagnostic radionuclide.
